(19) United States
(12) Patent Application Publication
(10) Pub. No.: US 2026/0345793 A1
(43) Pub. Date: Nov. 5, 2026

(54) BIFUNCTIONAL COMPOUNDS CONTAINING DIHYDROBENZO[D]ISOTHIAZOLE DERIVATIVES FOR DEGRADING CYCLIN-DEPENDENT KINASE 2 VIA UBIQUITIN PROTEASOME PATHWAY

(71) Applicant: BeOne Medicines I GmbH, Basel (CH)

(72) Inventors: Changxin Huo, Beijing (CN); Yu Zhao, Beijing (CN); Jie Gao, Beijing (CN); Huaqing Liu, Beijing (CN); Zhiwei Wang, Beijing (CN); Yuxi Wang, Beijing (CN)

(73) Assignee: BeOne Medicines I GmbH, Basel (CH)

(21) Appl. No.: 19/530,022

(22) Filed: Feb. 4, 2026

(30) Foreign Application Priority Data
Feb. 5, 2025 (WO) PCT/CN2025/075706
Jul. 30, 2025 (WO) PCT/CN2025/111461
Dec. 24, 2025 (WO) PCT/CN2025/145162

(51) Int. Cl. A61K 47/55; A61K 47/54; C07D 417/14

(52) U.S. Cl. CPC A61K 47/55; A61K 47/545; C07D 417/14

(57) ABSTRACT

Provided herein are compounds having the following structure:

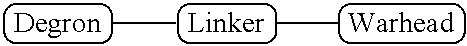

or a pharmaceutically acceptable salt, hydrate or solvate thereof,
wherein the Warhead is

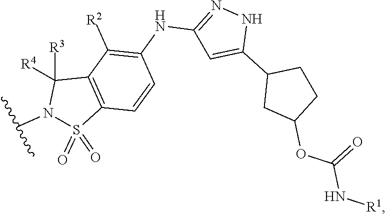

and the substituents are as defined herein, their pharmaceutical compositions and uses for modulating the activity of CDK2.

### RELATED APPLICATIONS

This application claims priority under 35 U.S.C. § 119 to International PCT applications, PCT/CN2025/145162, filed Dec. 24, 2025; PCT/CN2025/111461, filed Jul. 30, 2025; and PCT/CN2025/075706, filed Feb. 5, 2025, the entire contents of each of which is incorporated herein by reference.

### FIELD

Provided herein are certain bifunctional compounds that cause degradation of Cyclin-dependent kinase 2 (CDK2) via ubiquitin proteasome pathway and are therefore useful for the treatment of diseases mediated by CDK2. Also provided are processes for preparing such compounds.

### BACKGROUND

Cyclin-dependent kinases (CDKs) are cellular kinases that are critical for orchestrating signaling events such as DNA replication and protein synthesis to ensure faithful eukaryotic cell division and proliferation. To date, at least twenty-one mammalian CDKs have been identified. Among these CDKs, at least CDK1/Cyclin B, CDK2/Cyclin E, CDK2/Cyclin A, CDK4/Cyclin D, CDK6/Cyclin D complexes are known to be important regulators of cell cycle progression; while other CDKs are important in regulating gene transcription, DNA repair, differentiation, and apoptosis.

CCNE gene amplification or cyclinE overexpressed forms of cancer cells over activates CDK2, dysregulating Rb phosphorylation and resulting in cancer cell proliferation. Aberrant CCNE has been proved as a disease driver in multiple cancer types such as ovarian, esophageal, bladder, pancreatic and so on, which are associated with poor disease outcomes.

Proteolysis-targeting chimeric molecules (CDACs) are bifunctional molecules comprised of target protein-recruitment moiety and a ligand for E3 ligase, connected by a biocompatible linker. CDACs could bring the protein of interest and the E3 ligase into close proximity and induce ubiquitination and subsequent degradation of the target protein by proteasome. Compared to traditional small molecule drugs that typically bind disease-relevant proteins and inhibit their function, CDACs display several unique and attractive features that make them desirable drug candidates. CDACs have the ability to target previously undruggable proteins as they are not limited to binding of the catalytic domains.

In contract to inhibition, removal of CDK2 protein would eliminate CDK2 kinase activity as well as any protein interaction or scaffolding function of CDK2, which, for example, makes CDK2-CDAC as a potential candidate for treating CDK2-related but CDK2 inhibitor resistant medical conditions. Accordingly, there is a need for bifunctional molecules such as CDACs that could recruit CDK2 to a ubiquitin ligase, and thereby causing ubiquitylation and proteasomal degradation of CDK2. The present disclosure fulfills this and related needs.

Citation or identification of any reference in this section of this application is not to be construed as an admission that the reference is prior art to the present application.

### SUMMARY

Provided herein is a compound of formula (I):

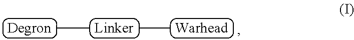

or a pharmaceutically acceptable salt, hydrate, or solvate thereof,
wherein
the Warhead is

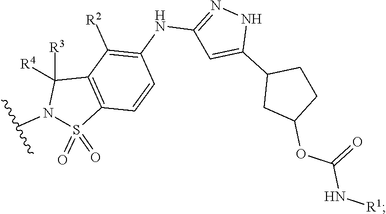

R¹ is C₁₋₄ alkyl or C₃₋₅cycloalkyl; R² is halogen; each of R³ and R⁴ is, independently, hydrogen or C₁₋₄ alkyl;
the Linker and Degron are defined as provided herein.

Also provided herein is a pharmaceutical composition comprising a compound of Formula (I) or a pharmaceutically acceptable salt, hydrate, or solvate thereof, and a pharmaceutically acceptable excipient.

Also provided herein is a method for modulating the activity of CDK2 protein in a cell, comprising contacting said cell with a therapeutically effective amount of a compound of Formula (I), or a pharmaceutically acceptable salt, hydrate, or solvate thereof.

Also provided herein is a method for treating cancer comprising administering to a subject in need thereof a therapeutically effective amount of a compound of formula (I), or a pharmaceutically acceptable salt, hydrate, or solvate thereof.

### DETAILED DESCRIPTION

### Definitions

Unless specifically defined elsewhere in this document, all other technical and scientific terms used herein have the meaning commonly understood by one of ordinary skill in the art.

It is meant to be understood that proper valences are maintained for all moieties and combinations thereof, that monovalent moieties having more than one atom are drawn from left to right and are attached through their left ends, and that divalent moieties are also drawn from left to right.

It is also meant to be understood that a specific embodiment of a variable moiety may be the same or different as another specific embodiment having the same identifier.

The indefinite articles “a” and “an” and the definite article “the” include plural as well as single referents, unless the context clearly indicates otherwise.

“Alkene” means an unsaturated hydrocarbon chain containing at least one carbon-carbon double bond (C═C). For examples, alkene includes, but is not limited to, alkenes with 2 to 20 carbon atoms, such as ethylene, propylene, butene, 1-butene (CH₂═CH—CH₂—CH₃), pentene, and their derivatives. The term “alkene” as used herein does not include a chain where the double bond is part of an aromatic ring system, such as in styrene or benzene derivatives.

“Alkyl” means a saturated straight or branched hydrocarbon chain having from 1 to 12 carbon atoms, typically from 1 to 8 carbon atoms, preferably from 1 to 6 carbon atoms, and more preferable from 1 to 4 carbon atoms. Representative alkyl groups include, but are not limited to, methyl, ethyl, n-propyl, n-butyl, n-pentyl, n-hexyl, isopropyl, sec-butyl, isobutyl, tert-butyl, isopentyl, neopentyl, tert-pentyl, 2-methylpentyl, 3-methylpentyl, 4-methylpentyl, 2,3-dimethylbutyl and the like.

“Alkoxy” or “alkoxyl” means —O-alkyl, wherein alkyl is defined herein.

“Amino” means a radical of the formula: —NH₂.

“Atropisomer” means a stereoisomer resulting from hindered rotation about a single bond axis where the rotational barrier is high enough to allow for the isolation of the individual rotational isomer.

“Bridged ring” means a ring system formed by connecting two non-adjacent atoms (usually carbon atoms) of a ring (mono- or fused multi cyclic) with an atom or chain of atoms. These two non-adjacent atoms are called “bridgeheads”. A bridged ring system may include only carbon atoms as ring atoms or may include carbon atoms and one to four heteroatoms (i.e., N, O, or S) as ring atoms. Examples of bridged ring systems include 1-bicyclo[1.1.1]pentyl, bicyclo[2.1.1]hexyl, bicyclo[2.2.1]heptyl, bicyclo[2.2.2]octyl, adamantanyl, norbornanyl, bicyclo [3.2.1] octyl, bicyclo [2.2.2]octyl, bicyclo[3.3.1]nonyl, bicyclo[3.2. .3]nonyl, 2-oxa-bicyclo [2.2.2] octyl, 1-aza-bicyclo [2.2.2]octyl, 3-aza-bicyclo [3.2.1]octyl, and 2, 6-dioxa-tricyclo [3.3.1.03,7] nonyl.

“Carbonyl” means a functional group consisting of a carbon atom double-bonded to an oxygen atom (C═O). A carbonyl group may be part of various chemical structures, including aldehydes, ketones, carboxylic acids, esters, amides, and anhydrides. A carbonyl group includes, but is not limited to, a functional group that may be present in organic compounds, including linear, branched, cyclic, or aromatic structures. A carbonyl group includes, but is not limited to, carbonyl groups present in organic compounds with 1 to 20 carbon atoms.

“Carboxy” means a radical of the formula: C(═O)OH.

“Cycloalkyl” or “cyclic ring” mean a saturated, monocyclic or multiple fused ring system having from 3 to 14 ring carbon atoms and no heteroatoms. Typically, the cycloalkyl or cyclic ring group is a monocyclic or bicyclic group having 3 to 10 ring carbon atoms, preferably a monocyclic group having 3 to 8 ring carbon atoms, more preferably a monocyclic group having 3 to 6 ring carbon atoms, and even more preferably a monocyclic group having 5 to 6 ring carbon atoms. Representative cycloalkyl or cyclic ring groups include, but are not limited to cyclopropyl, cyclobutyl, cyclopentyl, cyclohexyl, cycloheptyl, and cyclooctyl.

“Halogen” means fluorine, chlorine, bromine or iodine.

“Heterocyclyl” or “heterocyclic ring” means a non-aromatic monocyclic or multiple fused ring system in which at least one, preferably one to four, more preferably one to three, or even more preferably one to two of the ring carbon atoms are independently replaced with a heteroatom selected from the group consisting of O, S and N, wherein the nitrogen and sulfur heteroatoms may optionally be oxidized and/or the nitrogen heteroatom may be optionally be quarternized. The heterocyclyl or heterocyclic ring group can be saturated or unsaturated. Typically, the heterocyclyl or heterocyclic ring group is a monocyclic or bicyclic ring system having 3 to 14 ring atoms, preferably a monocyclic or bicyclic ring system having 3 to 10 ring atoms, more preferably a monocyclic ring having 3 to 6 ring atoms and even more preferably a monocyclic ring having 5 to 6 ring atoms. The heterocyclyl or heterocyclic ring group can be bonded to other groups at any ring atom (i.e., at any carbon atom or heteroatom of the heterocyclic ring system), provided that proper valences are maintained. Representative heterocyclyl or heterocyclic ring groups include, but are not limited to, aziridinyl, azetidinyl, azepanyl, oxetanyl, pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, dioxolyl, pyrrolinyl, imidazolinyl, pyrazolinyl, thiazolinyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dioxyl, dithianyl, pyranyl, dihydropyridyl, dihydrodithiinyl, dihydrodithionyl, 1,4-dioxaspiro[4.5]decanyl, 2-oxo-1-oxa-3,8-diazaspiro[4.5]decane, 1-oxo-2,8-diazaspiro[4.5]decane, 3-oxo-2,8-diazaspiro[4.5]decane, 3-oxo-1-oxa-4,9-diazaspiro[5.5]undecane, 2-oxo-1-oxa-3,9-diazaspiro[5.5]undecane, homopiperazinyl, quinuclidyl, indolinyl, isoindolinyl, quinolizinyl, dihydrobenzothiazinyl, dihydrobenzofuranyl, dihydroindolyl, dihydrobenzodioxinyl, tetrahydroquinolinyl groups, and the like.

“Hydrate” means a solvate wherein the solvent molecule is H₂O.

“Oxo” means ═O wherein the double bond is attached to a carbon, sulfur, or nitrogen.

“Pharmaceutically acceptable” means suitable for use in contact with the tissues of human beings without excessive toxicity, irritation, allergic response, or other problem or complication, and commensurate with a reasonable benefit/risk ratio.

“Pharmaceutically acceptable excipient” means a pharmaceutically acceptable inert ingredient or vehicle, such as a liquid or solid filler, diluent, excipient, solvent or encapsulating material. Each excipient must be “acceptable” in the sense of being compatible with the other ingredients of the formulation and not deleterious to the recipients thereof.

“Pharmaceutically acceptable salt(s)” means a salt prepared from a pharmaceutically acceptable non-toxic acid or base including an inorganic acid and base and an organic acid and base. The pharmaceutically acceptable salt used herein includes the solvate of the pharmaceutically acceptable salt thereof. In one embodiment, the pharmaceutically acceptable salt used herein includes the hydrate of the pharmaceutically acceptable salt thereof.

“Solvate” means a physical association of a compound of formula (I) or a pharmaceutically acceptable salt thereof with one or more pharmaceutically acceptable solvent molecules. This physical association involves varying degrees of ionic and covalent bonding, including hydrogen bonding. In certain instances, one or more solvent molecules are incorporated in the crystal lattice of the crystalline solid.

“Spiro ring” means a ring system that contains a bicyclic ring sharing a single ring atom (usually a quaternary carbon atom) between two ring members. Typically the spiro ring is a bicyclic ring system. The individual rings within a spiro ring system may be identical or different, may contain carbon atoms only as ring atoms, or may contain one to four heteroatoms as ring atom(s). Examples of spiro ring systems include spiropentane, spirohexane, spiro[5.4]decane, spiro[4.3]octane, spiro[5.2]octane, and spiropyran.

“Stereoisomer” or “stereomerically pure” means one stereoisomer of a compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound.

“Subject” means a human.

“Substituted” means that any one or more hydrogens on the designated atom or group is replaced with a selection (i.e., substituent) from the indicated groups, provided that the designated atom's normal valence is not exceeded. It will be understood by those skilled in the art, with respect to any group containing one or more substituents, that such groups are not intended to introduce any substitution or substitution patterns that are sterically impractical, synthetically non-feasible and/or inherently unstable.

“Tautomer” means an isomeric form of a compound that is in equilibrium with another isomeric form. The concentration of the isomeric form will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

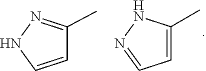

“Therapeutically effective amount” means an amount capable of treating a disorder, disease or condition, or symptoms thereof, disclosed herein.

“Treating” means alleviating, in whole or in part, of a disorder, disease or condition, or one or more of the symptoms associated with a disorder, disease, or condition, or slowing or halting of further progression or worsening of those symptoms, or alleviating the cause(s) of the disorder, disease, or condition itself.

### COMPOUNDS

The following aspects and embodiments are not intended to be an explicit or implicit admission that these aspects or embodiments are independent or distinct nor should it be interpreted as such. Rather, it is intended to convey information so that the full breadth of the present disclosure can be understood. Furthermore, the following aspects and embodiments are not meant to be limiting on the full breadth of the disclosure as recited herein.

Provided herein is a compound of formula (I):

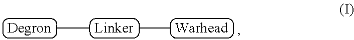

or a pharmaceutically acceptable salt, hydrate, or solvate thereof,
wherein
the Warhead is

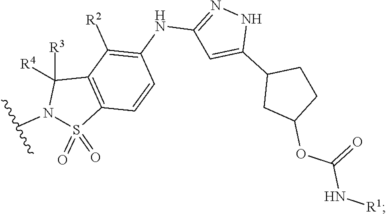

R¹ is C₁₋₄ alkyl or C₃₋₅cycloalkyl; R² is halogen; each of R³ and R⁴ is, independently, hydrogen or C₁₋₄ alkyl;
the Linker is

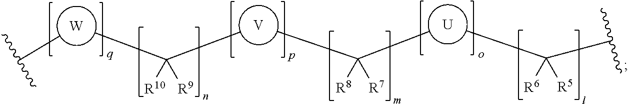

each of moieties U, V, and W is, independently, a 4 to 6 membered cyclic ring, 4 to 6 membered heterocyclic ring, 7 to 9 membered spiro heterocyclic ring, 5 to 8 membered bridged heterocyclic ring, or —N(R¹¹)—Z—, each of which is optionally substituted by one or more groups independently selected from halogen, —CN, —CF₃, —CHF₂, —CH₂F, C₁₋₄ alkyl, C₁₋₄ alkene, C₁₋₄ alkoxy and C₁₋₄ alkoxy-C₁₋₄ alkyl;
each of R⁵ and R⁶ is hydrogen, or R⁵, R⁶ and the carbon that R⁵ and R⁶ attach to form a carbonyl;
each of R⁷ and R⁸ is hydrogen, or R⁷, R⁸ and the carbon that R⁷ and R⁸ attach to form a carbonyl;
each of R⁹ and R¹⁰ is hydrogen, or R⁹, R¹⁰ and the carbon that R⁹ and R¹⁰ attach to form a carbonyl;
R¹¹ is hydrogen or C₁₋₄ alkyl;
Z is a 4 to 6 membered cyclic ring;
each of 1, m, n, o, p, and q is, independently, 0 or 1; the sum of o, p, and q is 1, 2, or 3;
the Degron is

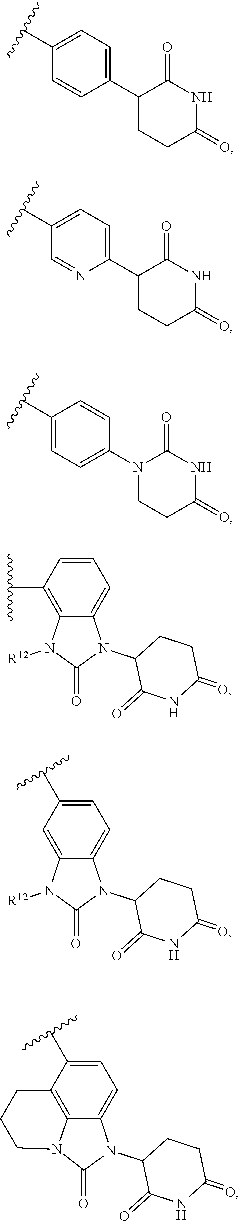

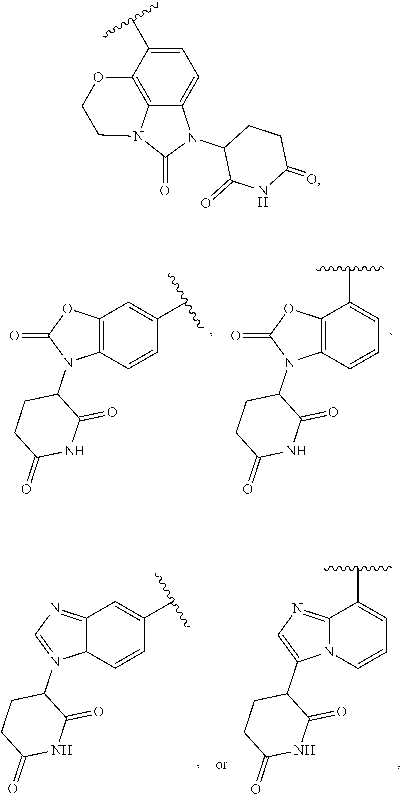

each of which is optionally substituted by one or more groups independently selected from halogen, —CN, —CH₂F, —CHF₂, —CF₃, C₁₋₄ alkyl, C₁₋₄ alkoxy, —OCF₃, C₃₋₅cycloalkyl and 3 to 5 membered heterocyclyl; and R¹² is hydrogen or C₁₋₄ alkyl.

In certain embodiments, the Linker is

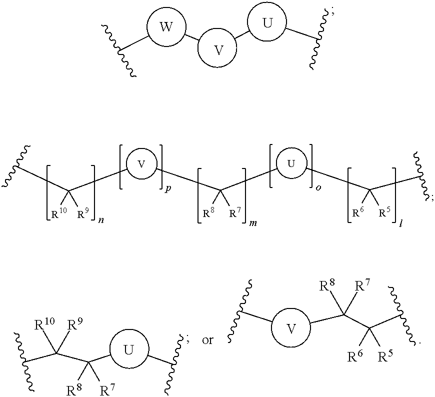

In certain embodiments, R¹ is C₁₋₄ alkyl; R² is F or Cl; each of R³ and R⁴ is hydrogen; each of moieties U, V, and W is, independently, a 4 to 6 membered cyclic ring, 4 to 6 membered heterocyclic ring, 7 to 9 membered spiro heterocyclic ring, 5 to 8 membered bridged heterocyclic ring or —N(R¹¹)—Z—, each of which is optionally substituted by one or more groups selected from halogen, —CN, —CF₃, —CHF₂, —CH₂F, C₁₋₄ alkyl, C₁₋₄ alkene, C₁₋₄ alkoxy and C₁₋₄ alkoxy-C₁₋₄ alkyl;
R⁵ and R⁶ are hydrogen;
R⁷ and R⁸ are hydrogen;
R⁹ and R¹⁰ are hydrogen;
the Degron is

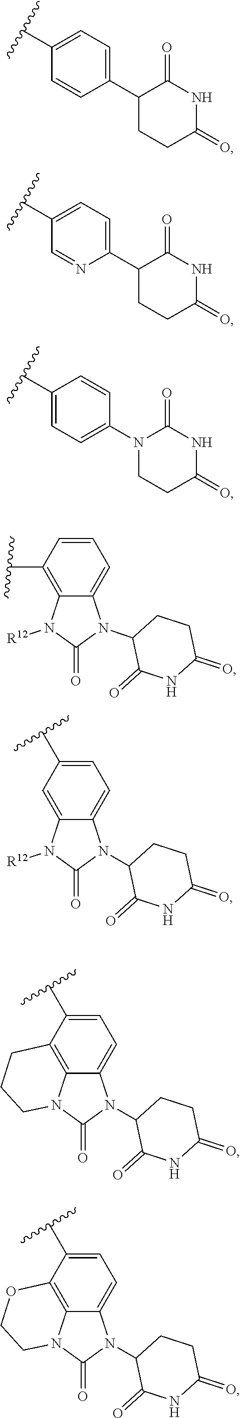

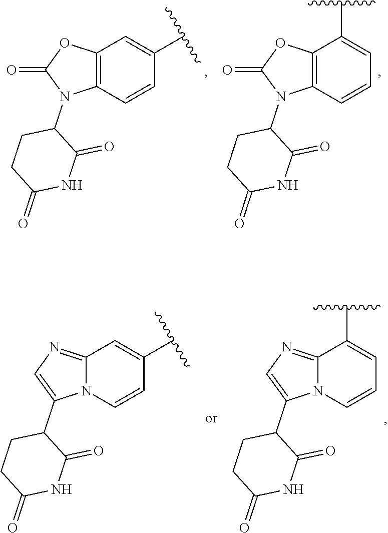 [SMILES: *c1ccc2c(c1)oc(=O)n2C1CCC(=O)NC1=O ; *c1cccc2c1oc(=O)n2C1CCC(=O)NC1=O ; *c1ccn2c(C3CCC(=O)NC3=O)cnc2c1 ; *c1cccn2c(C3CCC(=O)NC3=O)cnc12]

each of which is optionally substituted by one or more groups independently selected from F, —CN, —CH₂F, —CHF₂, —CF₃, C₁₋₄ alkyl, C₁₋₄ alkoxy, —OCF₃ and C₃₋₅cycloalkyl; and R¹² is hydrogen or C₁₋₄ alkyl.

In certain embodiments, the Linker is

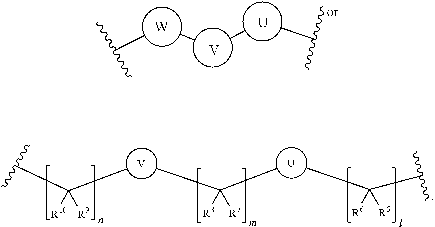

In certain embodiments, each of moieties U, V and W is, independently,

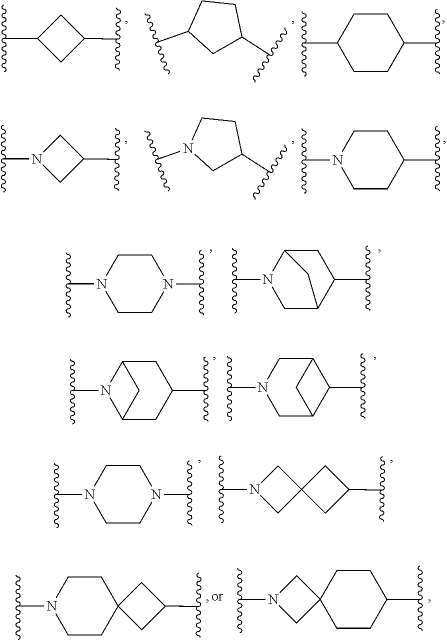

each of which is optionally substituted by one or more groups independently selected from halogen, —CN, —CH₂F, —CHF₂, —CF₃, C₁₋₄ alkyl, C₁₋₄ alkene, C₁₋₄ alkoxy and C₁₋₄ alkoxy-C₁₋₄ alkyl.

In certain embodiments, each of moieties U, V and W is, independently, optionally substituted by one or more groups selected from F, —CH₂F, methyl, methylene, methoxy and methoxy-methyl.

In certain embodiments, each of moieties U, V and W is, independently, optionally substituted by one or more groups selected from F and methyl.

In certain embodiments, R¹ is C₁₋₄ alkyl; R² is F; and R³ and R⁴ are hydrogen.

In certain embodiments, the Degron is

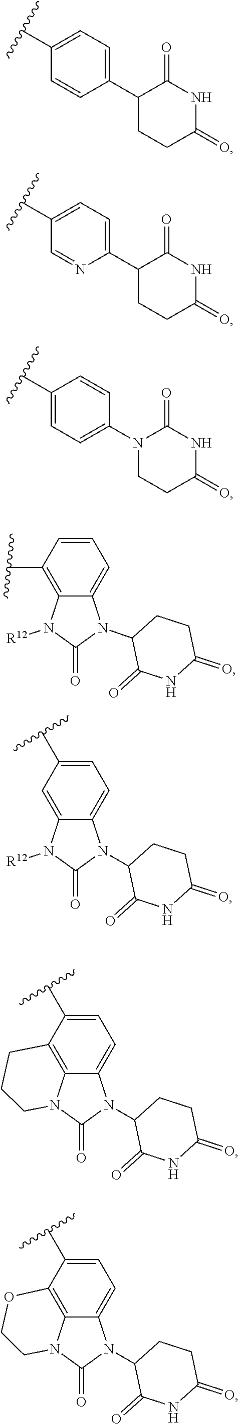

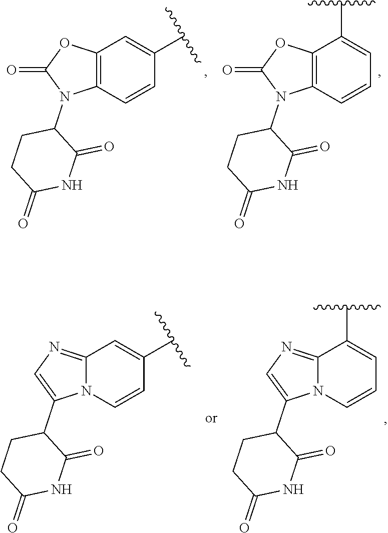 [SMILES: *c1ccc2c(c1)oc(=O)n2C1CCC(=O)NC1=O ; *c1cccc2c1oc(=O)n2C1CCC(=O)NC1=O ; *c1ccn2c(C3CCC(=O)NC3=O)cnc2c1 ; *c1cccn2c(C3CCC(=O)NC3=O)cnc12]

each of which is optionally substituted by one or more groups independently selected from F, —CN, —CH₂F, —CHF₂, —CF₃, C₁₋₄ alkyl, C₁₋₄ alkoxy and —OCF₃; and R¹² is hydrogen, methyl or isopropyl.

In certain embodiments, the Linker is

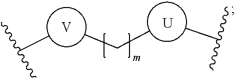

and each of moieties U and V is, independently,

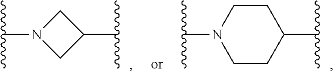 [SMILES: *C1CN(*)C1 ; *C1CCN(*)CC1]

each of which is optionally substituted by one or two groups independently selected from F and methyl.

In certain embodiments, the Linker is

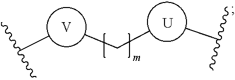

and
each of moieties U, and V is, independently,

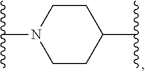 [SMILES: *C1CCN(*)CC1]

optionally substituted by one or two groups independently selected from F and methyl.

In certain embodiments, m is 1.

In certain embodiments, the Degron is

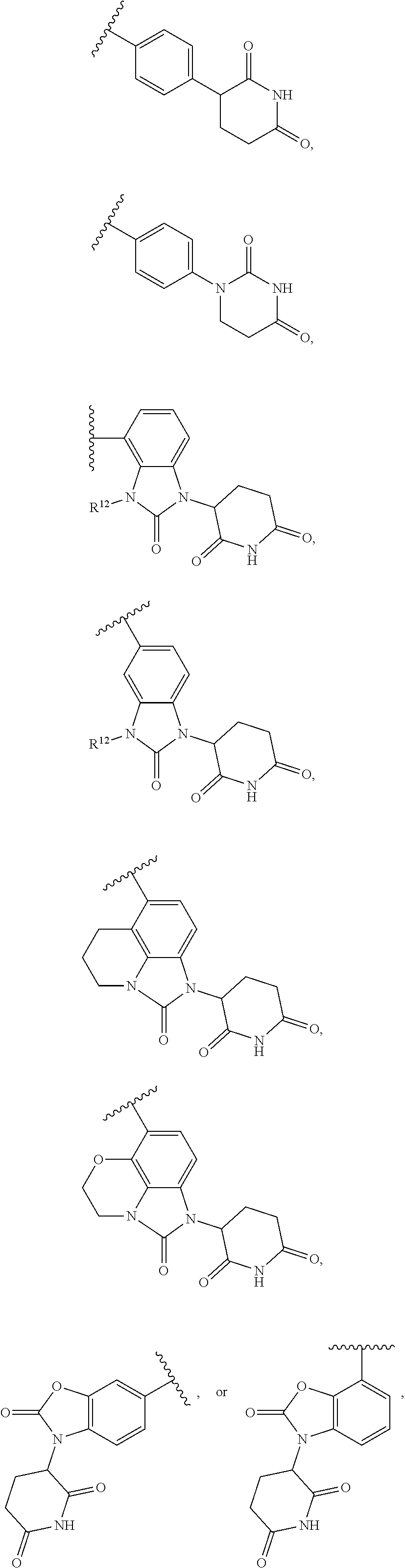

each of which is optionally substituted by one or two groups independently selected from F and methyl.

In certain embodiments, R¹ is isopropyl or tert-butyl; R² is F; and R³, and R⁴ are hydrogen.

In certain embodiments, R¹ is isopropyl.

In certain embodiments, R¹ is tert-butyl.

In certain embodiments, R¹² is methyl, or isopropyl.

In certain embodiments, R¹² is methyl.

In certain embodiments, R¹² is isopropyl.

In certain embodiments, the Degron is

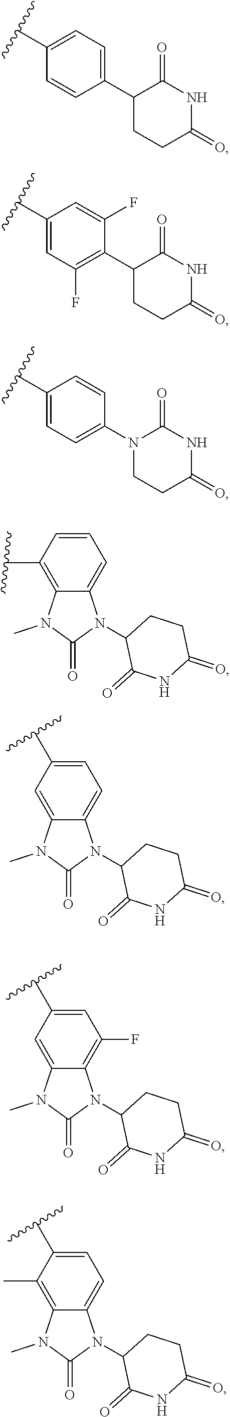

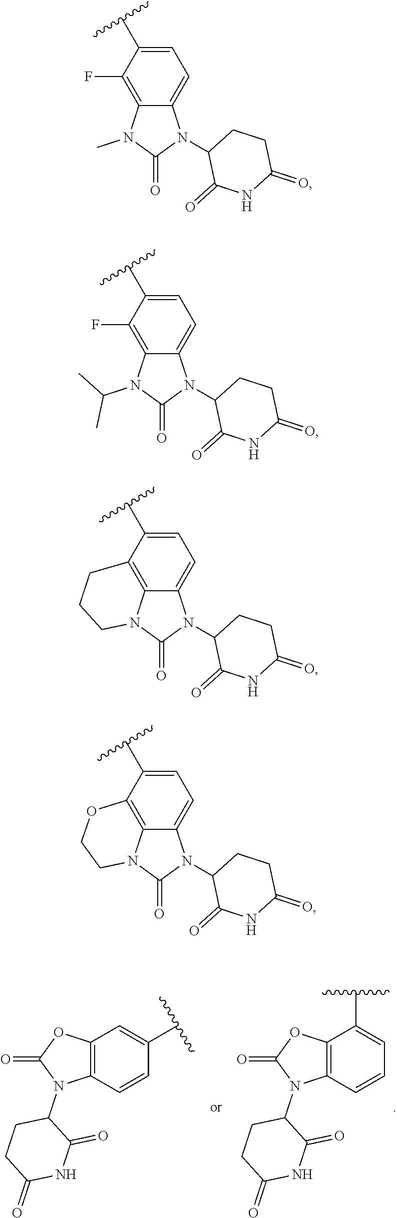 [SMILES: *c1ccc2c(c1F)n(C)c(=O)n2C1CCC(=O)NC1=O ; *c1ccc2c(c1F)n(C(C)C)c(=O)n2C1CCC(=O)NC1=O ; *c1ccc2c3c1CCCn3c(=O)n2C1CCC(=O)NC1=O ; *c1ccc2c3c1OCCn3c(=O)n2C1CCC(=O)NC1=O ; *c1ccc2c(c1)oc(=O)n2C1CCC(=O)NC1=O ; *c1cccc2c1oc(=O)n2C1CCC(=O)NC1=O]

In certain embodiments, the Degron is

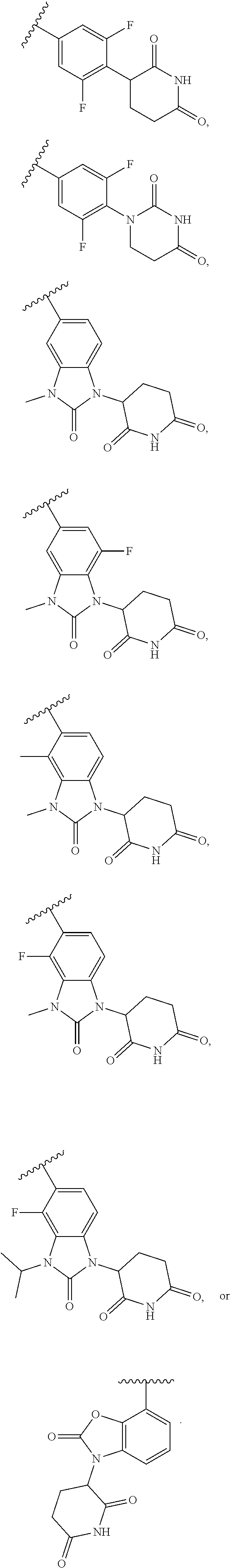

In certain embodiments, the compound is selected from the examples provided herein.

In certain embodiments, provided herein is a pharmaceutical composition comprising a compound provided herein, or a pharmaceutically acceptable salt thereof, and a pharmaceutically acceptable excipient.

In certain embodiments, provided herein is a method of treating a disease mediated by CDK2 in a patient comprising administering to the patient in need thereof, a therapeutically effective amount of a compound provided herein, or a pharmaceutical composition provided herein. In an embodiment of any one of above aspects, CDK2 is selectively degraded over CDK1. In another embodiment of any one of above aspects, CDK2 is selectively degraded over CDK1 and CDK4 and/or CDK6. In certain embodiments, the disease is cancer.

In certain embodiments, provided herein is a method of treating cancer in a patient comprising administering to the patient in need thereof, a therapeutically effective amount of a compound provided herein, or a pharmaceutically acceptable salt thereof, or a pharmaceutical composition provided herein.

The compound of Formula (I) may contain asymmetric or chiral centers, and therefore, exist in different stereoisomeric forms, such as enantiomers, diastereomers, or atropisomers. All stereoisomeric forms of the compounds of Formula (I) as well as mixtures thereof, including racemic mixtures, form part of the present invention.

It should also be noted that the compound of Formula (I) can include E and Z isomers, or a mixture thereof, and cis and trans isomers or a mixture thereof.

It is also possible that the compound of Formula (I) may exist in different tautomeric forms, and all such forms are within the scope of the present invention.

In the compound of Formula (I), the atoms may exhibit their natural abundances, or one or more of the atoms may be artificially enriched in a particular isotope having the same atomic number, but an atomic mass or mass number different from the atomic mass or mass number predominantly found in nature. All suitable isotopic variations of the compound of Formula (I) are within the scope of the present invention. For example, different isotopic forms of hydrogen (H) include protium (¹H) and deuterium (²H).

### NUMBERED EMBODIMENTS

In Embodiment 1, provided herein is a compound of formula (I):

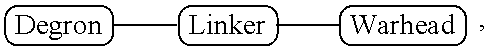

or a pharmaceutically acceptable salt, hydrate, or solvate thereof,
wherein the Warhead is

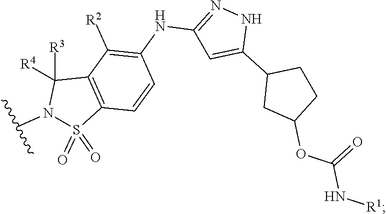

R¹ is C₁₋₄ alkyl or C₃₋₅cycloalkyl; R² is halogen; each of R³ and R⁴ is, independently, hydrogen, or C₁₋₄ alkyl;
the Linker is

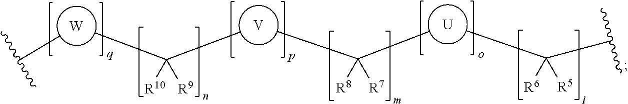

each of moieties U, V, and W is, independently, a 4 to 6 membered cyclic ring, 4 to 6 membered heterocyclic ring, 7 to 9 membered spiro heterocyclic ring, 5 to 8 membered bridged heterocyclic ring, or —N(R¹¹)—Z—, each of which is optionally substituted by one or more groups selected from halogen, —CN, —CF₃, —CHF₂, —CH₂F, C₁₋₄ alkyl, C₁₋₄ alkene, C₁₋₄ alkoxy and C₁₋₄ alkoxy-C₁₋₄ alkyl;
each of R⁵ and R⁶ is independently hydrogen, or R⁵, R⁶ and the carbon that R⁵ and R⁶ attach to form a carbonyl;
each of R⁷ and R⁸ is independently hydrogen, or R⁷, R⁸, and the carbon that R⁷ and R⁸ attach to form a carbonyl;
each of R⁹ and R¹⁰ is independently hydrogen, or R⁹, R¹⁰ and the carbon that R⁹ and R¹⁰ attach to form a carbonyl;
R¹¹ is hydrogen or C₁₋₄ alkyl;
Z is a 4 to 6 membered cyclic ring;
each of 1, m, n, o, p and q is, independently, 0 or 1; the sum of o, p, and q is 1, 2 or 3;
the Degron is

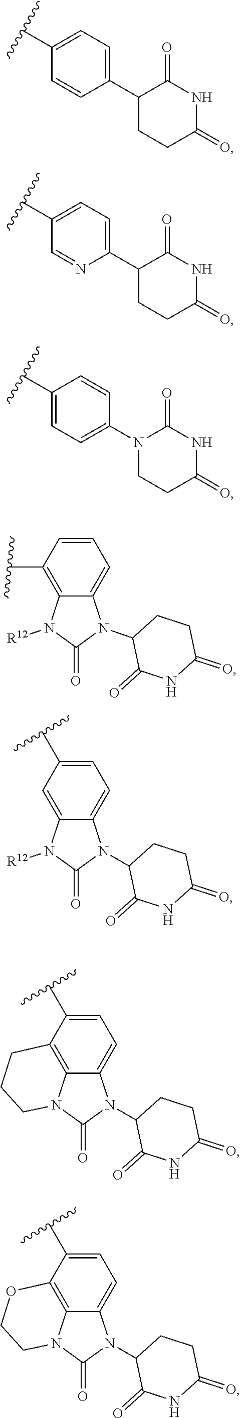

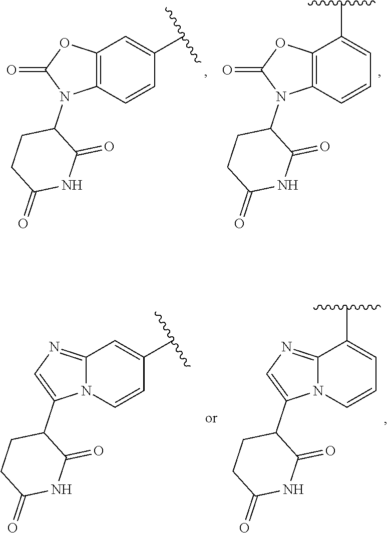 [SMILES: *c1ccc2c(c1)oc(=O)n2C1CCC(=O)NC1=O ; *c1cccc2c1oc(=O)n2C1CCC(=O)NC1=O ; *c1ccn2c(C3CCC(=O)NC3=O)cnc2c1 ; *c1cccn2c(C3CCC(=O)NC3=O)cnc12]

each of which is optionally substituted by one or more groups selected from halogen, —CN, —CH₂F, —CHF₂, —CF₃, C₁₋₄ alkyl, C₁₋₄ alkoxy, —OCF₃, C₃₋₅cycloalkyl and 3 to 5 membered heterocyclyl; and R¹² is hydrogen, or C₁₋₄ alkyl.

In Embodiment 2, provided herein is the compound of Embodiment 1, wherein the Linker is

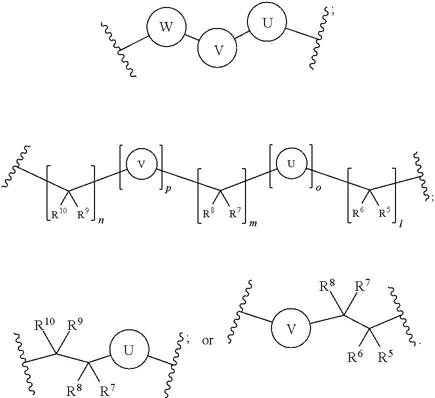

In Embodiment 3, provided herein is the compound of Embodiment 2, wherein R¹ is C₁₋₄ alkyl; R² is F or Cl; each of R³ and R⁴ is hydrogen; each of moieties U, V, and W is, independently, a 4 to 6 membered cyclic ring, 4 to 6 membered heterocyclic ring, 7 to 9 membered spiro heterocyclic ring, 5 to 8 membered bridged heterocyclic ring, or —N(R¹¹)—Z—, each of which is optionally substituted by one or more groups selected from halogen, —CN, —CF₃, —CHF₂, —CH₂F, C₁₋₄ alkyl, C₁_4 alkene, C₁₋₄ alkoxy, and C₁₋₄ alkoxy-C₁₋₄ alkyl;
R⁵ and R⁶ are hydrogen;
R⁷ and R⁸ are hydrogen;
R⁹ and R¹⁰ are hydrogen;
the Degron is

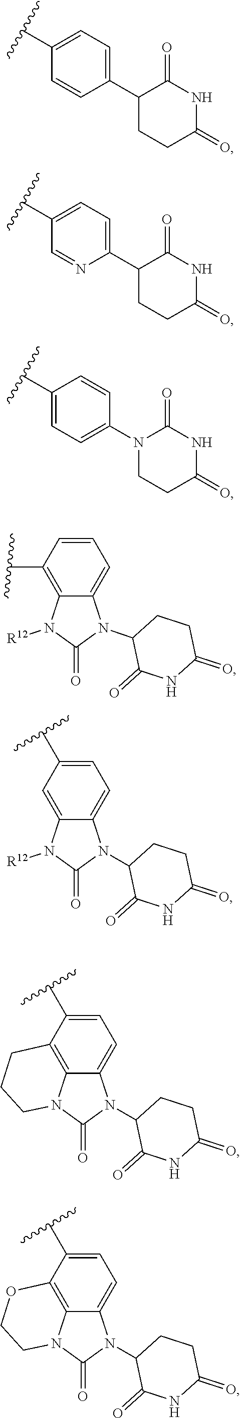

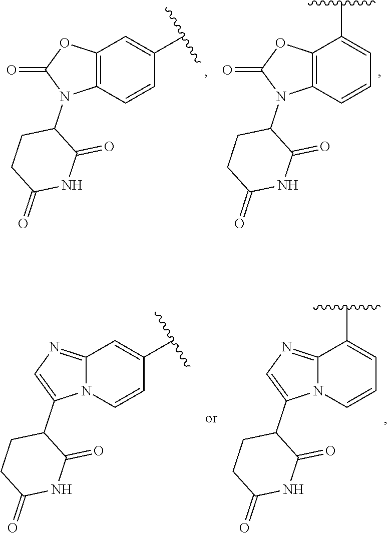 [SMILES: *c1ccc2c(c1)oc(=O)n2C1CCC(=O)NC1=O ; *c1cccc2c1oc(=O)n2C1CCC(=O)NC1=O ; *c1ccn2c(C3CCC(=O)NC3=O)cnc2c1 ; *c1cccn2c(C3CCC(=O)NC3=O)cnc12]

each of which is optionally substituted by one or more groups selected from F, —CN, —CH₂F, —CHF₂, —CF₃, C₁4 alkyl, C₁4 alkoxy, —OCF₃ and C₃₋₅cycloalkyl; and R¹² is hydrogen, or C₁₋₄ alkyl.

In Embodiment 4, provided herein is the compound of Embodiment 3, wherein the Linker is

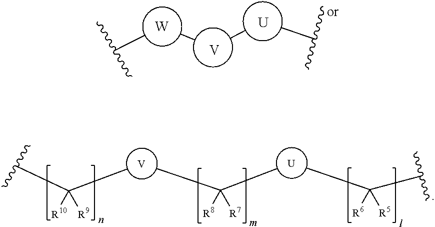

In Embodiment 5, provided herein is the compound of Embodiment 4, wherein each of moieties U, V and W is, independently,

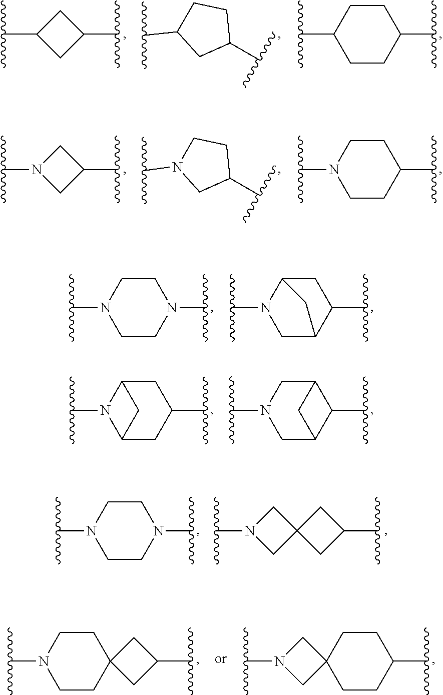

each of which is optionally substituted by one or more groups selected from halogen, —CN, —CH₂F, —CHF₂, —CF₃, C₁₋₄ alkyl, C₁₋₄ alkene, C₁₋₄ alkoxy and C₁₋₄ alkoxy-C₁₋₄ alkyl.

In Embodiment 6, provided herein is the compound of Embodiment 5, wherein each of moieties U, V and W is, independently, optionally substituted by one or more groups selected from F, —CH₂F, methyl, methylene, methoxy and methoxy-methyl.

In Embodiment 7, provided herein is the compound of Embodiment 6, wherein each of moieties U, V and W is, independently, optionally substituted by one or more groups selected from F or methyl.

In Embodiment 8, provided herein is the compound of Embodiment 7, wherein R¹ is C₁₋₄ alkyl; R² is F; and R³ and R⁴ are hydrogen.

In Embodiment 9, provided herein is the compound of Embodiment 8, wherein the Degron is

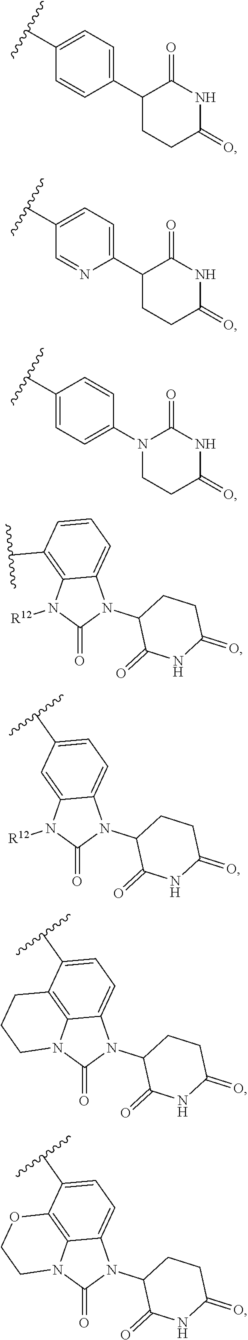

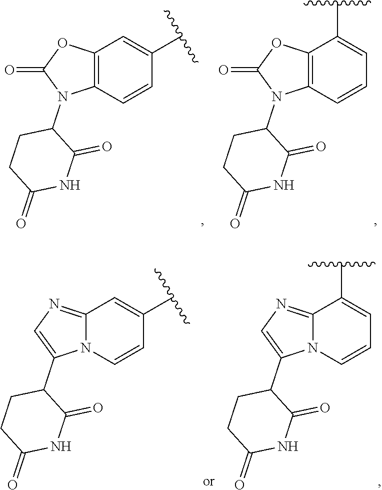 [SMILES: *c1ccc2c(c1)oc(=O)n2C1CCC(=O)NC1=O ; *c1cccc2c1oc(=O)n2C1CCC(=O)NC1=O ; *c1ccn2c(C3CCC(=O)NC3=O)cnc2c1 ; *c1cccn2c(C3CCC(=O)NC3=O)cnc12]

each of which is optionally substituted by one or more groups selected from F, —CN, —CH₂F, —CHF₂, —CF₃, C₁₋₄ alkyl, C₁₋₄ alkoxy and —OCF₃; and R¹² is hydrogen, methyl, or isopropyl.

In Embodiment 10, provided herein is the compound of Embodiment 9, wherein the Linker is

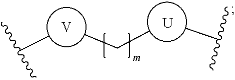

and
each of moieties U, and V is, independently,

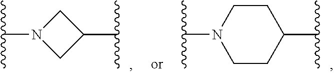 [SMILES: *C1CN(*)C1 ; *C1CCN(*)CC1]

each of which is optionally substituted by one or two groups selected from F and methyl.

In Embodiment 11, provided herein is the compound of Embodiment 9, wherein the Linker is

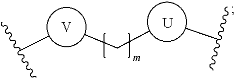

and
each of moieties U, and V is, independently,

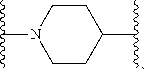 [SMILES: *C1CCN(*)CC1]

optionally substituted by one or two groups selected from F and methyl.

In Embodiment 12, provided herein is the compound of Embodiment 11, wherein m is 1.

In Embodiment 13, provided herein is the compound of Embodiment 10, wherein the Degron is

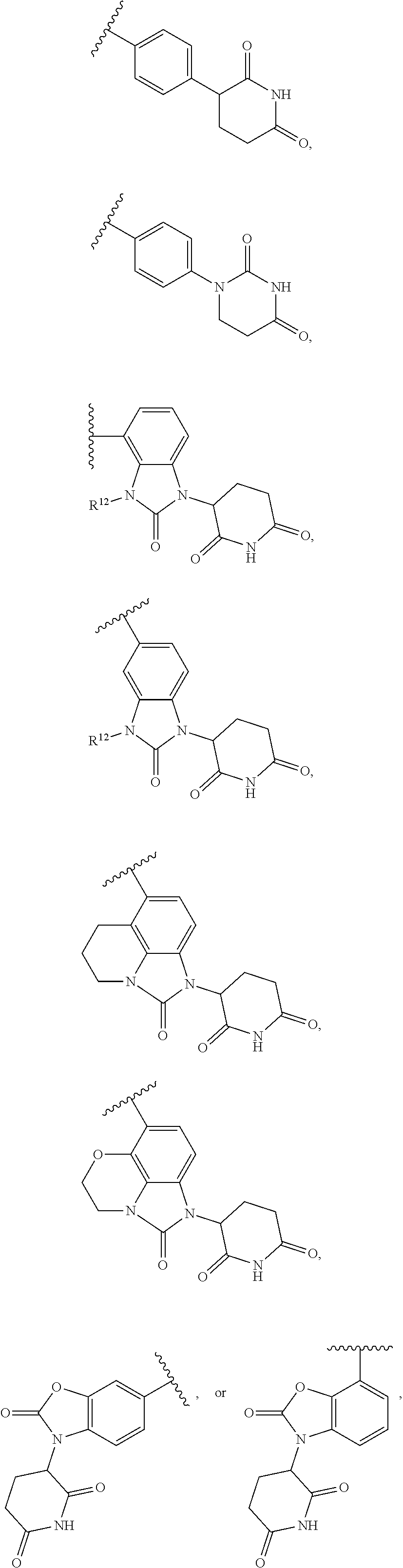

each of which is optionally substituted by one or two groups selected from F and methyl.

In Embodiment 14, provided herein is the compound of Embodiment 13, wherein R¹ is isopropyl or tert-butyl; R² is F; and R³, and R⁴ are hydrogen.

In Embodiment 15, provided herein is the compound of Embodiment 14, wherein R¹ is isopropyl.

In Embodiment 16, provided herein is the compound of Embodiment 14, wherein R¹ is tert-butyl.

In Embodiment 17, provided herein is the compound of Embodiment 14, wherein R¹² is methyl, or isopropyl.

In Embodiment 18, provided herein is the compound of Embodiment 14, wherein R¹² is methyl.

In Embodiment 19, provided herein is the compound of Embodiment 14, wherein R¹² is isopropyl.

In Embodiment 20, provided herein is the compound of Embodiment 17, wherein the Degron is

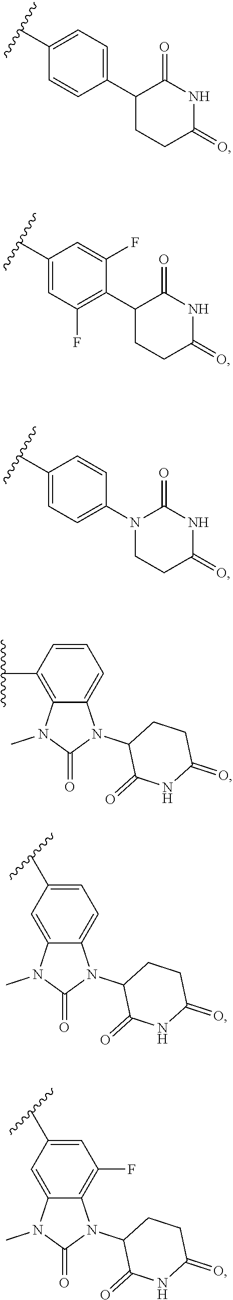 [SMILES: *c1ccc(C2CCC(=O)NC2=O)cc1 ; *c1cc(F)c(C2CCC(=O)NC2=O)c(F)c1 ; *c1ccc(N2CCC(=O)NC2=O)cc1 ; *c1cccc2c1n(C)c(=O)n2C1CCC(=O)NC1=O ; *c1ccc2c(c1)n(C)c(=O)n2C1CCC(=O)NC1=O ; *c1cc(F)c2c(c1)n(C)c(=O)n2C1CCC(=O)NC1=O]

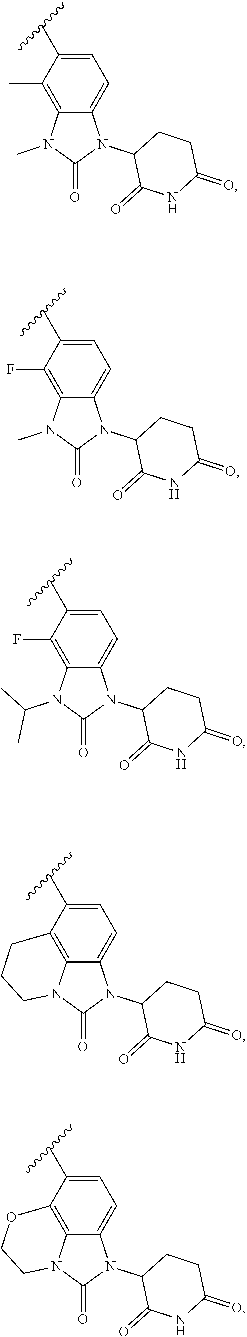 [SMILES: *c1ccc2c(c1C)n(C)c(=O)n2C1CCC(=O)NC1=O ; *c1ccc2c(c1F)n(C)c(=O)n2C1CCC(=O)NC1=O ; *c1ccc2c(c1F)n(C(C)C)c(=O)n2C1CCC(=O)NC1=O ; *c1ccc2c3c1CCCn3c(=O)n2C1CCC(=O)NC1=O ; *c1ccc2c3c1OCCn3c(=O)n2C1CCC(=O)NC1=O]

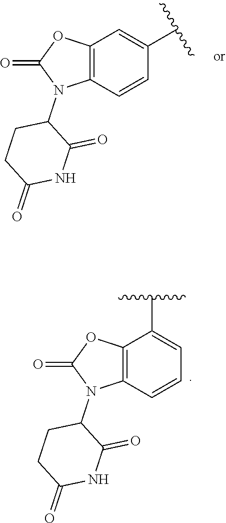 [SMILES: *c1ccc2c(c1)oc(=O)n2C1CCC(=O)NC1=O ; *c1cccc2c1oc(=O)n2C1CCC(=O)NC1=O]

In Embodiment 21, provided herein is the compound of Embodiment 20, wherein the Degron is

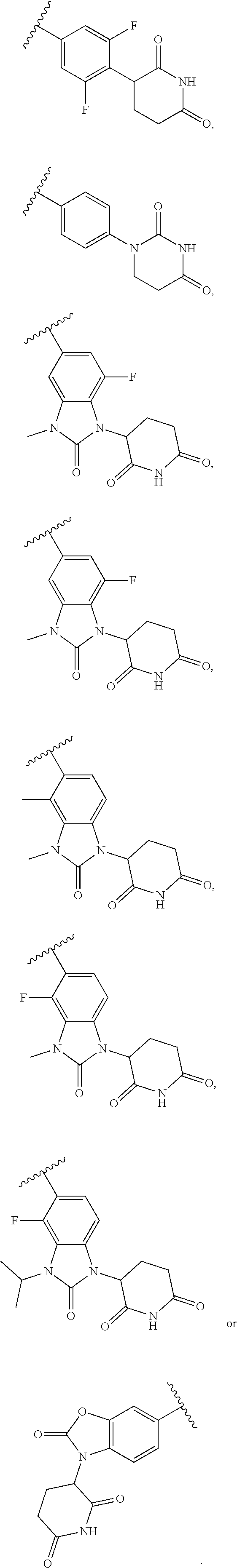

### METHODS FOR MAKING COMPOUNDS

The Compounds can be made using conventional organic syntheses and commercially available starting materials. By way of example and not limitation, Compounds of Formula (I) can be prepared as outlined in the scheme shown below as well as in the examples set forth herein. It should be noted that one skilled in the art would know how to modify the procedures set forth in the illustrative schemes and examples to arrive at the desired products. Common protecting groups may be used to prevent certain functional groups from undergoing undesired reaction. Exemplary protecting groups are described in “Protective Groups in Organic Synthesis”, 4^{th} Edition, P. G. M. Wuts; T. W. Greene, John Wiley, 2007, and references cited therein.

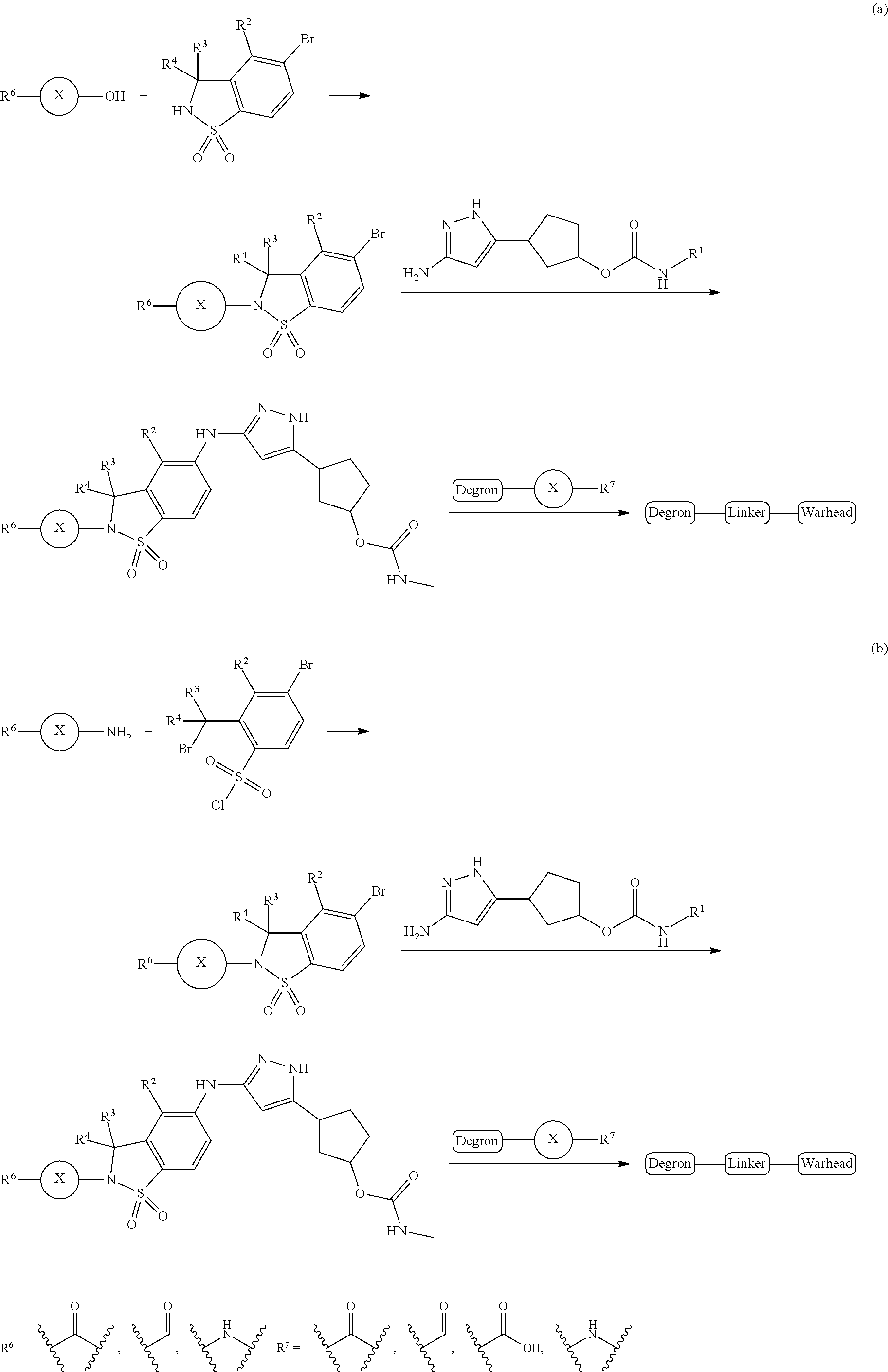

### EXAMPLES

The examples below are intended to be purely exemplary and should not be considered to be limiting in any way. Unless otherwise specified, the experimental methods in the Examples described below are conventional methods. Unless otherwise specified, the reagents and materials are all commercially available. All solvents and chemicals employed are of analytical grade or chemical purity. Solvents are all redistilled before use. Anhydrous solvents are all prepared according to standard methods or reference methods.

In the following examples, the following abbreviations are used:

| | |
|---|---|
| Brine | Saturated aqueous sodium chloride solution |
| Bn | Benzyl |
| BnBr | Benzyl Bromide |
| Boc | Tert-butoxycarbonyl |
| CH₂Cl₂ or DCM | Dichloromethane |
| Cs₂CO₃ | Cesium carbonate |
| DMF | N,N-Dimethylformamide |
| DIEA or DIPEA | N,N-diisopropylethylamine |
| DMAP | 4-N,N-dimethylaminopyridine |
| Dess-Martin/DMP | Dess-Martin Periodinane |
| DMF-DMA | N,N-Dimethylformamide dimethyl acetal purum |
| DMSO | Dimethyl sulfoxide |
| EtOAc or EA | Ethyl acetate |
| EtOH | Ethanol |
| Et₂O or ether | Diethyl ether |
| g | Grams |
| h or hr | Hour |
| HATU | O-(7-Azabenzotriazol-1-y1)- |
| | N,N,N′,N′-tetramethyluronium |
| | hexafluorophosphate |
| HCl | Hydrochloric acid |
| HMDS | Hexamethyldisilazane |
| HPLC | High-performance liquid chromatography |
| IBX | 2-Iodylbenzoic acid |
| i-PrOH | Isopropyl alcohol |
| LCMS | Liquid chromatography-mass spectrometry |
| LiHMDS | Lithium Bis(trimethylsilyl)amide |
| K₂OsO₄•H2O | Potassium osmate(VI) dihydrate |
| K₃PO₄ | Tripotassium phosphate |
| mg | Milligrams |
| mL | Milliliters |
| mmol | Millimole |
| MeCN/ACN | Acetonitrile |
| MeOH | Methanol |
| Min | Minutes |
| ms or MS | Mass spectrum |
| Na₂SO₄ | Sodium sulfate |
| NaH | Sodium hydride |
| NaHMDS | Sodium bis(trimethylsilyl)amide |
| NBS | N-Bromosuccinimide |
| NH₄Cl | Ammonium chloride |
| PD | Pharmacodynamic(s) |
| PE | petroleum ether |
| PK | Pharmacokinetic(s) |
| POCl₃ | phosphorous oxychloride |
| PddppfCl₂ | hexafluorophosphate |
| Pd(dtbpf)Cl₂ | [1,1′- |
| Pd₂(dba)₃ | Bis(diphenylphosphino)ferrocene]di- |
| | chloropalladium(II) |
| Pd(PPh₃)₄ | [1,1′-Bis(di-tert- |
| | butylphosphino)ferrocene]dichloropalladium(II) |
| | Tris(dibenzylideneacetone)dipalladium |
| | Tetrakis (triphenylphosphine)palladium(0) |
| Prep | Preparative |
| Rt or rt | Room temperature |
| RuPhos | 2-Dicyclohexylphosphino- |
| | 2′,6′-diisopropoxybiphenyl |
| sat. | Saturated |
| TEA/Et3N | triethylamine |
| t-BuOK | Potassium tert-butoxide |
| t-BuONa | Sodium tert-butoxide |
| TFA | Trifluoroacetic acid |
| THF | Tetrahydrofuran |
| TLC | thin layer chromatography |
| μL | Microliters |
| Xphos | 2-Dicyclohexylphosphino-2′,4′,6′- |
| | triisopropylbiphenyl |

### EXAMPLES

### Example 1: (1S,3R)-3-(3-((2-(1-((1-(4-((R)-2,6-dioxopiperidin-3-yl)-3,5-difluorophenyl) piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

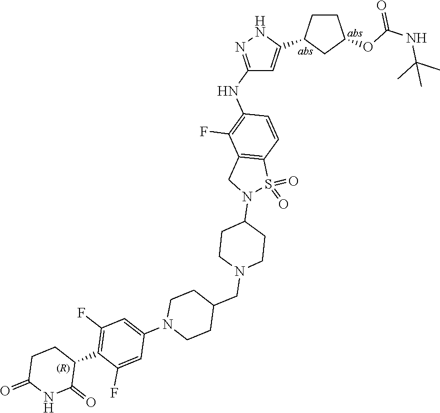

### Step 1: benzyl (1-(tert-butyl)-3-((1R,3S)-3-(((4-nitrophenoxy)carbonyl)oxy)cyclopentyl)-1H-pyrazol-5-yl)carbamate

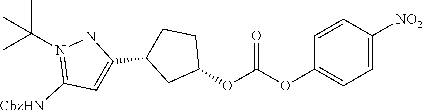

To a solution of benzyl (1-(tert-butyl)-3-((1R,3S)-3-hydroxycyclopentyl)-1H-pyrazol-5-yl)carbamate (83 g, 233 mmol) in DCM (1000 mL) was added pyridine (92 g, 1165 mmol), DMAP (2.8 g, 23 mmol), and 4-nitrophenyl carbonochloridate (49 g, 244 mmol), successively. The resulting mixture was stirred at room temperature for 16 h. The reaction mixture was concentrated under reduced pressure, and the residue was purified by silica gel chromatography, eluting with PE/EA (3:1) to afford the product (112 g, 92% yield). LC-MS (ESI): m/z [M+H]⁺=523.2

### Step 2: benzyl (5-((1R,3S)-3-(((4-nitrophenoxy)carbonyl)oxy)cyclopentyl)-1H-pyrazol-3-yl)carbamate

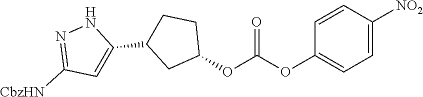

To a flask was added benzyl (1-(tert-butyl)-3-((1R,3S)-3-(((4-nitrophenoxy)carbonyl) oxy)cyclopentyl)-1H-pyrazol-5-yl)carbamate (112 g, 214 mmol) and formic acid (1200 mL). The mixture was stirred at 80° C. for over night. The solvent was removed under reduced pressure to obtain the crude product. LC-MS (ESI): m/z [M+H]⁺=467.2.

### Step 3: benzyl (5-((1R,3S)-3-((tert-butylcarbamoyl)oxy)cyclopentyl)-1H-pyrazol-3-yl)carbamate

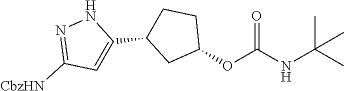 [SMILES: CC(C)(C)NC(=O)O[C@H]1CC[C@@H](c2cc(NC(=O)OCc3ccccc3)n[nH]2)C1]

To a solution of benzyl (5-((1R,3S)-3-(((4-nitrophenoxy)carbonyl)oxy)cyclopentyl)-1H-pyrazol-3-yl)carbamate (100 g, 215 mmol) in THF (1000 mL) was added tert-butylamine (78.5 g, 1075 mmol) and DIEA (138.7 g, 1075 mmol) at 0° C. The resulting mixture was stirred at room temperature for 3 h. The mixture was filtered, and the filtrate was poured into water. The mixture was extracted by EA (500 mL×3), and the combined organic phase was dried over Na-₂SO₄ and concentrated under reduced pressure. The residue was purified by silica gel chromatography (PE/EA=1:1) to afford the product (70.5 g, 85% yield). LC-MS (ESI): m/z [M+H]⁺=401.4

### Step 4: (1S,3R)-3-(3-amino-1H-pyrazol-5-yl)cyclopentyl tert-butylcarbamate

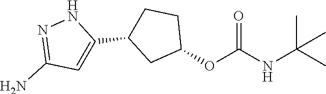 [SMILES: CC(C)(C)NC(=O)O[C@H]1CC[C@@H](c2cc(N)n[nH]2)C1]

To a solution of benzyl (5-((1R,3S)-3-((tert-butylcarbamoyl)oxy)cyclopentyl)-1H-pyrazol-3-yl)carbamate (20 g, 50 mmol) in THF (100 mL) was added Pd/C (10%, wet, 15 g). The suspension was degassed and purged with hydrogen 3 times. The resulting mixture was stirred at room temperature under a hydrogen balloon for 3 h. The mixture was filtered, and the filter cake was washed with EA (50 mL×3) and methanol (50 mL×3). The filtrate was concentrated under reduced pressure to afford the product (10.5 g, 78.9% yield). LC-MS (ESI): m/z [M+H]⁺=267.2

### Step 5: methyl 6-amino-3-bromo-2-fluorobenzoate

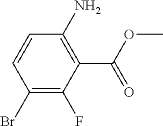

To a solution of 6-amino-3-bromo-2-fluorobenzoic acid (1.0 g, 4.27 mmol, Vendor Chemscene, Cat #CS-0060313) in methanol (20 mL) was added SOCl₂ (2.5 g, 21.3 mmol) dropwise. The reaction mixture was stirred under refluxed for 16 hours. The resulting mixture was cooled to room temperature, concentrated, neutralized with sat. aq sodium bicarbonate and extracted with EA (100 mL×3). The combined organic layers were washed with brine, dried over anhydrous sodium sulfate, and concentrated under vacuum. The residue was purified by silica gel column chromatography, eluting with PE/EA (5:1) to afford the product (346 mg, 33%). LC-MS (ESI): m/z [M+H]⁺=249.0.

### Step 6: methyl 3-bromo-6-(chlorosulfonyl)-2-fluorobenzoate

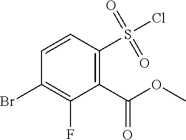 [SMILES: COC(=O)c1c(S(=O)(=O)Cl)ccc(Br)c1F]

To a suspension of methyl 6-amino-3-bromo-2-fluorobenzoate (3.0 g, 12.1 mmol) in hydrochloric acid (12 M, 60 mL), a solution of sodium nitrite (835 mg, 12.1 mmol) in 5 mL water was added dropwise at 0° C. The resulting mixture was kept below 5° C. and stirred for 30 mins after addition. Then a solution of sodium bisulfite (18.9 g, 181.5 mmol) in hydrochloric acid (5.5 M, 20 mL) was added dropwise followed by CuCl₂ (813 mg, 6.05 mmol). The reaction mixture was warmed to room temperature and stirred for 3 h. The reaction mixture was poured into ice water (50 mL) and extracted with EA (50 mL×3). The combined organic layers were washed with brine, dried over anhydrous sodium sulfate, filtered, and concentrated in vacuum to afford the crude product (3.2 g, 79% yield, LC-MS (ESI): m/z [M+H]⁺=332.5), which was used in the next step without further purification.

### Step 7: 5-bromo-4-fluorobenzo[d]isothiazol-3(2H)-one 1,1-dioxide

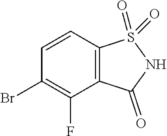 [SMILES: O=C1NS(=O)(=O)c2ccc(Br)c(F)c21]

To a stirred solution of methyl 3-bromo-6-(chlorosulfonyl)-2-fluorobenzoate (3.2 g, 9.6 mmol) in THF (50 mL) was added aqueous ammonia (25%, 10 mL) dropwise. The resulting solution was stirred at room temperature for 16 h. The resulting mixture was concentrated under vacuum, and the residue was triturated with EA and filtered to afford the crude product (2.4 g, 90% yield, LC-MS (ESI): m/z [M+H]⁺=281.1).

### Step 8: 5-bromo-4-fluoro-2,3-dihydrobenzo[d]isothiazole 1,1-dioxide

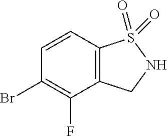 [SMILES: O=S1(=O)NCc2c1ccc(Br)c2F]

To a stirring solution of 5-bromo-4-fluorobenzo[d]isothiazol-3(2H)-one 1,1-dioxide (210 mg, 0.75 mmol) in THF (5 mL) was added BH₃-Me₂S (2 M, 1.5 mL, 3.0 mmol) dropwise at 0° C. under nitrogen atmosphere. The solution was heated to reflux and stirred for 3 h. The resulting mixture was cooled to 0° C., quenched with MeOH (1.0 mL), and concentrated under reduced pressure. The residue was purified by silica gel column chromatography, eluting with PE/EA (1:1) to afford the product (184 mg, 93%). LC-MS (ESI): m/z [M+H]⁺=265.8

### Step 9: tert-butyl 4-(5-bromo-4-fluoro-1,1-dioxidobenzo[d]isothiazol-2(3H)-yl)piperidine-1-carboxylate

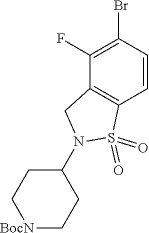 [SMILES: CC(C)(C)OC(=O)N1CCC(N2Cc3c(ccc(Br)c3F)S2(=O)=O)CC1]

To a stirring solution of 5-bromo-4-fluoro-2,3-dihydrobenzo[d]isothiazole 1,1-dioxide (10 g, 37.7 mmol) in toluene (200 mL) was added tert-butyl 4-hydroxypiperidine-1-carboxylate (11.4 g, 56.6 mmol) and 2-(tributylphosphoranylidene)acetonitrile (27.3 g, 113.1 mmol) at room temperature. The resulting solution was heated to 100° C. and stirred under nitrogen atmosphere for overnight. The solution was concentrated under reduced pressure, then triturated with PE/EA (4:1, 200 mL) and filtered to afford the product (11.1 g, 65.7% yield). LC-MS (ESI): m/z [M+H]⁺=449.1

### Step 10: tert-butyl 4-(5-((5-((1R,3S)-3-((tert-butylcarbamoyl)oxy)cyclopentyl)-1H-pyrazol-3-yl)amino)-4-fluoro-1,1-dioxidobenzo[d]isothiazol-2(3H)-yl)piperidine-1-carboxylate

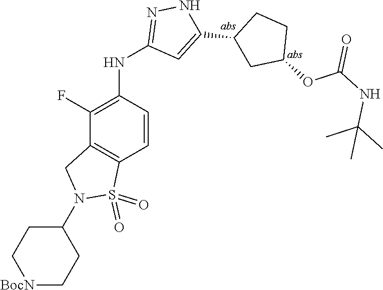 [SMILES: CC(C)(C)NC(=O)O[C@H]1CC[C@@H](c2cc(Nc3ccc4c(c3F)CN(C3CCN(C(=O)OC(C)(C)C)CC3)S4(=O)=O)n[nH]2)C1]

To a mixture of tert-butyl 4-(5-bromo-4-fluoro-1,1-dioxidobenzo[d]isothiazol-2(3H)-yl)piperidine-1-carboxylate (448 mg, 1 mmol) and (1S,3R)-3-(3-amino-1H-pyrazol-5-yl)cyclopentyl tert-butylcarbamate (266 mg, 1 mmol) in 2-methylpropan-2-ol (10 mL) was added BrettPhos Pd G3 (46 mg, 0.05 mmol) and K₂CO₃ (276 mg, 2 mmol). The reaction mixture was stirred under nitrogen atmosphere at 90° C. for 16 h. The mixture was filtered, and the filtrate was concentrated under reduced pressure. The residue was purified by silica gel column chromatography, eluting with DCM/MeOH (10:1) to afford the product (501 mg, 79% yield). LC-MS (ESI): m/z [M+H]⁺=635.3.

### Step 11: (1S,3R)-3-(3-((4-fluoro-1,1-dioxido-2-(piperidin-4-yl)-2,3-dihydrobenzo[d]isothiazol-5-yl)amino)-1H-pyrazol-5-yl)cyclopentyl tert-butylcarbamate

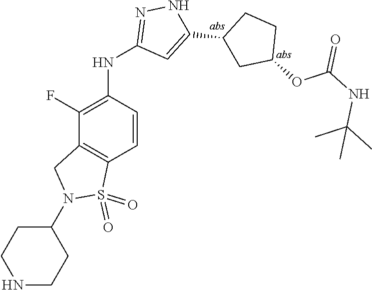 [SMILES: CC(C)(C)NC(=O)O[C@H]1CC[C@@H](c2cc(Nc3ccc4c(c3F)CN(C3CCNCC3)S4(=O)=O)n[nH]2)C1]

To a flask was added tert-butyl 4-(5-((5-((1R,3S)-3-((tert-butylcarbamoyl)oxy) cyclopentyl)-1H-pyrazol-3-yl)amino)-4-fluoro-1,1-dioxidobenzo[d]isothiazol-2(3H)-yl)piperidine-1-carboxylate (500 mg, 0.79 mmol) and 4N HCl in dioxane (5 mL). The resulting solution was stirred at room temperature for 1 h. The mixture was concentrated under reduced pressure to get the crude product without further purification. LC-MS (ESI): m/z [M+H]⁺=535.5.

### Step 12: (1S,3R)-3-(3-((2-(1-((1-(4-((R)-2,6-dioxopiperidin-3-yl)-3,5-difluorophenyl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

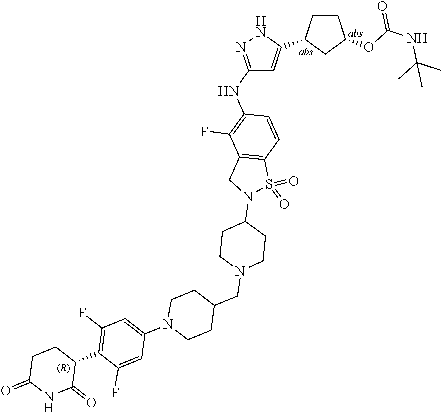

To a solution of (1S,3R)-3-(3-((4-fluoro-1,1-dioxido-2-(piperidin-4-yl)-2,3-dihydrobenzo[d]isothiazol-5-yl)amino)-1H-pyrazol-5-yl)cyclopentyl tert-butylcarbamate (93 mg, 0.17 mmo) in DMA (5 mL) was added (R)-1-(4-(2,6-dioxopiperidin-3-yl)-3,5-difluorophenyl)piperidine-4-carbaldehyde (58 mg, 0.19 mmol) and STAB (74 mg, 0.35 mmol). The mixture was stirred at rt for 4 h. The mixture was concentrated under reduced pressure, and the residue was purified by silica gel chromatography (DCM/MeOH=8:1) to afford the product (70 mg, 85% purity). The residue was purified by preparative HPLC (Column: Xselect C18, 19*150 mm, 5 μm; Mobile Phase A: H2O (0.1% FA), Mobile Phase B: ACN; Flow Rate: 17 mL/min; Room Temperature; Detector: UV 214 & 254 nm; Gradient: 27 to 47% B in 11 min, 47% to 90% B in 0.2 min, 90% B hold 2 min, 90% to 27% B in 0.3 min, 27% B hold 1.5 min.) to afford the product (55 mg, 38% yield). LC-MS (ESI): m/z [M+H]⁺=855.1.

¹H NMR (500 MHz, DMSO-d₆) δ 11.98 (s, 1H), 10.86 (s, 1H), 9.62 (s, 1H), 8.77 (d, J=3 Hz, 1H), 8.28 (m, 1H), 7.5 (d, J=9 Hz, 1H), 6.76 (s, 1H), 6.62-6.60 (m, 2H), 5.83 (s, 1H), 5.02-4.94 (m, 1H), 4.51 (s, 2H), 4.04 (dd, J₁=13 Hz, J₂=5.5 Hz 1H), 3.79-3.71 (m, 2H), 3.47-3.39 (m, 1H), 3.09-3.02 (m, 1H), 2.94-2.85 (m, 2H), 2.81-2.69 (m, 3H), 2.46-2.44 (m, 1H), 2.20-2.13 (m, 2H), 2.11-1.81 (m, 10H), 1.79-1.66 (m, 5H), 1.63-1.53 (m, 1H), 1.2 (s, 9H), 1.17-1.08 (m, 2H).

### Example 2 and Example 3: (1S,3R)-3-(3-((2-(((1s,4s)-4-(4-(4-((R)-2,6-dioxopiperidin-3-yl)-3,5-difluorophenyl)piperazin-1-yl)cyclohexyl)methyl)-4-fluoro-1,1-dioxido-2,3-dihydrobenzo[d]isothiazol-5-yl)amino)-1H-pyrazol-5-yl)cyclopentyl tert-butylcarbamate and (1S,3R)-3-(3-((2-(((1r,4r)-4-(4-(4-((R)-2,6-dioxopiperidin-3-yl)-3,5-difluorophenyl)piperazin-1-yl)cyclohexyl)methyl)-4-fluoro-1,1-dioxido-2,3-dihydrobenzo[d]isothiazol-5-yl)amino)-1H-pyrazol-5-yl)cyclopentyl tert-butylcarbamate

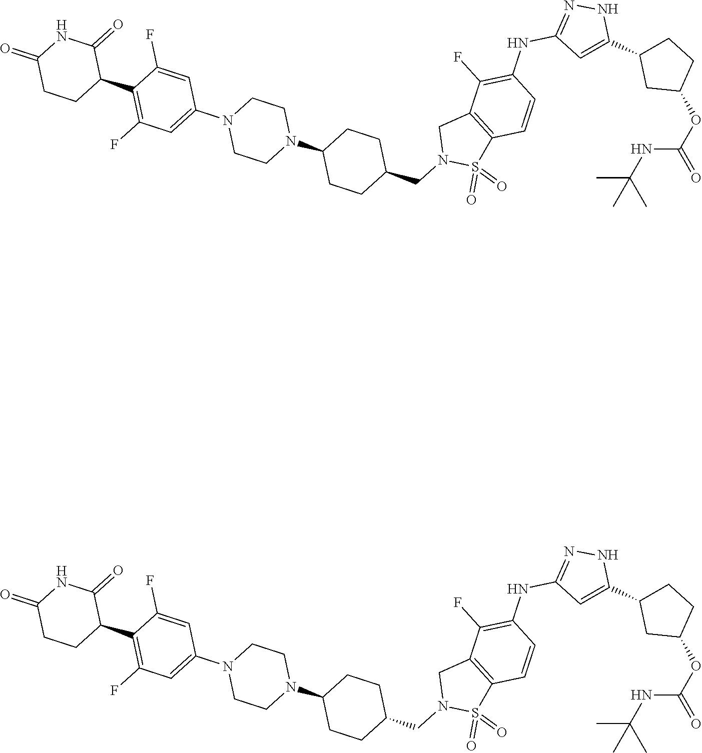 [SMILES: CC(C)(C)NC(=O)O[C@H]1CC[C@@H](c2cc(Nc3ccc4c(c3F)CN(C[C@H]3CC[C@@H](N5CCN(c6cc(F)c([C@H]7CCC(=O)NC7=O)c(F)c6)CC5)CC3)S4(=O)=O)n[nH]2)C1 ; CC(C)(C)NC(=O)O[C@H]1CC[C@@H](c2cc(Nc3ccc4c(c3F)CN(C[C@H]3CC[C@H](N5CCN(c6cc(F)c([C@H]7CCC(=O)NC7=O)c(F)c6)CC5)CC3)S4(=O)=O)n[nH]2)C1]

### Step 1: (R)-3-(2,6-difluoro-4-(piperazin-1-yl)phenyl)piperidine-2,6-dione

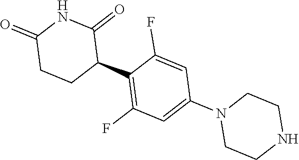 [SMILES: O=C1CC[C@H](c2c(F)cc(N3CCNCC3)cc2F)C(=O)N1]

To a solution of tert-butyl (R)-4-(4-(2,6-dioxopiperidin-3-yl)-3,5-difluorophenyl) piperazine-1-carboxylate (100 mg, 0.244 mmol) in dichloromethane (3 mL) was added 2,2,2-trifluoroacetic acid (0.5 mL), the reaction mixture was stirred for 1 hour at room temperature. The resulting mixture was concentrated, diluted with dichloromethane, washed with saturated aqueous NaHCO₃ and brine, then dried over anhydrous Na₂SO₄, then filtered and concentrated under reduced pressure to afford the product (70 mg, 93% yield). LC-MS (ESI): m/z [M+H]⁺=310.2.

### Step 2: (1S,3R)-3-(3-((2-((1,4-dioxaspiro[4.5]decan-8-yl)methyl)-4-fluoro-1,1-dioxido-2,3-dihydrobenzo[d]isothiazol-5-yl)amino)-1H-pyrazol-5-yl)cyclopentyl tert-butylcarbamate

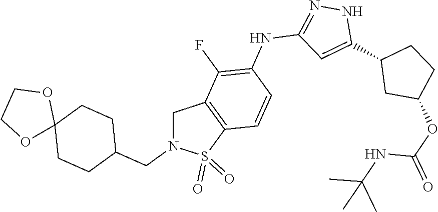 [SMILES: CC(C)(C)NC(=O)O[C@H]1CC[C@@H](c2cc(Nc3ccc4c(c3F)CN(CC3CCC5(CC3)OCCO5)S4(=O)=O)n[nH]2)C1]

To a mixture of 2-((1,4-dioxaspiro[4.5]decan-8-yl)methyl)-5-bromo-4-fluoro-2,3-dihydrobenzo[d]isothiazole 1,1-dioxide (300 mg, 0.71 mmol) and (1S,3R)-3-(3-amino-1H-pyrazol-5-yl)cyclopentyl tert-butylcarbamate (190 mg, 0.71 mmol) in 2-methylpropan-2-ol (10 mL) was added BrettPhos Pd G3 (65 mg, 0.07 mmol) and K₂CO₃ (300 mg, 2.14 mmol). The reaction mixture was stirred under nitrogen atmosphere at 90° C. for 16 h. The mixture was filtered, and the filtrate was concentrated under reduced pressure. The residue was purified by silica gel column chromatography, eluting with DCM/MeOH (10:1) to afford the product (355 mg, 82% yield). LC-MS (ESI): m/z [M+H]⁺=635.5

### Step 3: (1S,3R)-3-(3-((4-fluoro-1,1-dioxido-2-((4-oxocyclohexyl)methyl)-2,3-dihydrobenzo[d]isothiazol-5-yl)amino)-1H-pyrazol-5-yl)cyclopentyl tert-butylcarbamate

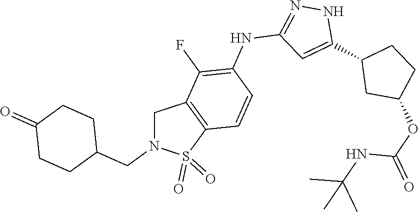 [SMILES: CC(C)(C)NC(=O)O[C@H]1CC[C@@H](c2cc(Nc3ccc4c(c3F)CN(CC3CCC(=O)CC3)S4(=O)=O)n[nH]2)C1]

(1S,3R)-3-(3-((2-((1,4-dioxaspiro[4.5]decan-8-yl)methyl)-4-fluoro-1,1-dioxido-2,3-dihydrobenzo[d]isothiazol-5-yl)amino)-1H-pyrazol-5-yl)cyclopentyl tert-butylcarbamate (355 mg, 0.59 mmol) was dissolved in formic acid (5 mL), the reaction mixture was stirred for 16 hours at room temperature. The resulting mixture was concentrated under reduce pressure, dried over in vacuum to afford the product (330 mg, 100% yield). LC-MS (ESI): m/z [M+H]⁺=562.2.

### Step 4: (1S,3R)-3-(3-((2-((cis-4-(4-(4-((R)-2,6-dioxopiperidin-3-yl)-3,5-difluorophenyl)piperazin-1-yl)cyclohexyl)methyl)-4-fluoro-1,1-dioxido-2,3-dihydrobenzo[d]isothiazol-5-yl)amino)-1H-pyrazol-5-yl)cyclopentyl tert-butylcarbamate and (1S,3R)-3-(3-((2-((trans-4-(4-(4-((R)-2,6-dioxopiperidin-3-yl)-3,5-difluorophenyl)piperazin-1-yl)cyclohexyl)methyl)-4-fluoro-1,1-dioxido-2,3-dihydrobenzo[d]isothiazol-5-yl)amino)-1H-pyrazol-5-yl)cyclopentyl tert-butylcarbamate

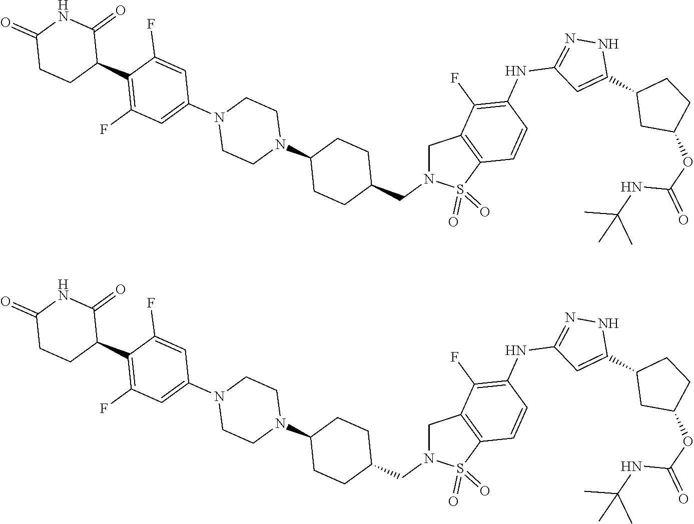 [SMILES: CC(C)(C)NC(=O)O[C@H]1CC[C@@H](c2cc(Nc3ccc4c(c3F)CN(C[C@H]3CC[C@@H](N5CCN(c6cc(F)c([C@H]7CCC(=O)NC7=O)c(F)c6)CC5)CC3)S4(=O)=O)n[nH]2)C1 ; CC(C)(C)NC(=O)O[C@H]1CC[C@@H](c2cc(Nc3ccc4c(c3F)CN(C[C@H]3CC[C@H](N5CCN(c6cc(F)c([C@H]7CCC(=O)NC7=O)c(F)c6)CC5)CC3)S4(=O)=O)n[nH]2)C1]

(1S,3R)-3-(3-((4-fluoro-1,1-dioxido-2-((4-oxocyclohexyl)methyl)-2,3-dihydrobenzo[d]isothiazol-5-yl)amino)-1H-pyrazol-5-yl)cyclopentyl tert-butylcarbamate (80 mg, 0.14 mmol) and (R)-3-(2,6-difluoro-4-(piperazin-1-yl)phenyl)piperidine-2,6-dione (44 mg, 0.14 mmol) were dissolved in N,N-dimethylacetamide (3 mL), stirred for 30 minutes, sodium triacetoxyborohydride (60 mg, 0.28 mmol) was added, the reaction mixture was stirred for 16 hours at room temperature. The resulting mixture was concentrated, the residue was purified by column chromatography on silica gel, eluting with DCM/MeOH (10:1) to afford the product (45 mg, 38%) in a racemic form, which was further separated by Chiral Prep-HPLC to give:

Enantiomer 1 (Example 2, 100% ee); Retention time: 5.072 min. ¹H NMR (500 MHz, DMSO) δ=11.92 (s, 1H), 10.80 (s, 1H), 8.71 (d, J=2.0, 1H), 8.23 (s, 1H), 7.47 (d, J=8.7, 1H), 6.69 (s, 1H), 6.56 (d, J=12.6, 2H), 5.77 (s, 1H), 4.91 (s, 1H), 4.41 (s, 2H), 3.99 (dd, J=12.5, 5.0, 1H), 3.12 (s, 4H), 2.99 (m, 3H), 2.77-2.67 (m, 1H), 2.50 (m, 4H), 2.46 (m, 1H), 2.41-2.36 (m, 1H), 2.17 (m, 1H), 2.08-1.93 (m, 2H), 1.86 (m, 3H), 1.70-1.57 (m, 4H), 1.55-1.39 (m, 7H), 1.15 (s, 9H). LC-MS (ESI): m/z [M+H]⁺=855.6.

Enantiomer 2 (Example 3, 100% ee); Retention time: 8.195 min. ¹H NMR (500 MHz, DMSO) δ=11.99 (s, 1H), 10.87 (s, 1H), 8.78 (s, 1H), 8.30 (s, 1H), 7.53 (d, J=8.6, 1H), 6.76 (s, 1H), 6.62 (d, J=12.6, 2H), 5.83 (s, 1H), 4.98 (s, 1H), 4.48 (s, 2H), 4.05 (dd, J=12.5, 4.9, 1H), 3.16 (s, 4H), 3.07 (m, 1H), 2.96 (d, J=7.0, 2H), 2.82-2.74 (m, 1H), 2.62 (m, 4H), 2.45 (m, 1H), 2.30 (m, 1H), 2.05 (m, 2H), 1.91 (m, 6H), 1.77-1.70 (m, 2H), 1.62 (m, 2H), 1.30-1.16 (m, 3H), 1.17 (s, 9H), 0.98 (m, 2H). LC-MS (ESI): m/z [M+H]⁺=855.6.

Chiral analytical method: Column: ICHIRALPAK IB N-5 4.6*150 mm 5 μm; Mobile phase: A for MtBE (0.1%2M NH3 MeOH) (%) and B for MeOH:DCM=50:50(%); Gradient: Mobile Phase A: Mobile Phase B=60:40 (v/v); HPLC Equipment: HPLC-Agilent, Back pressure: 100 bar; Column temperature: 35° C.

Chiral Prep-HPLC Condition: Column: CHIRALPAK®IE, 250*20.0 mml, 5 μm; Mobile phase: A for MtBE and B for DCM:MeOH=1:1; Gradient: Mobile Phase A: Mobile Phase B=20:80 (v/v); Flow Rate: 18 mL/min, Wave Length: UV 250 nm and 290 nm, Prep-HPLC Equipment: Prep-HPLC Gilson (ARD_LC-815), Back pressure: 100 bar; Column temperature: 25° C.

### Example 4: (1S,3R)-3-(3-((2-(1-((1-(3-((R)-2,6-dioxopiperidin-3-yl)-2,4-difluorophenyl) azetidine-3-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamatete

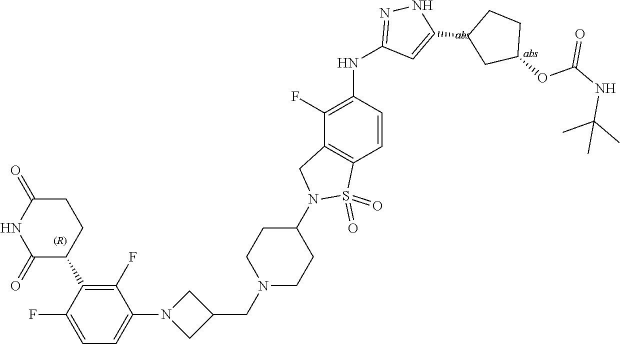

The titled compound was synthesized in the procedures similar to Example 1. ¹H NMR (500 MHz, DMSO-d₆) δ 11.99 (s, 1H), 10.92 (s, 1H), 8.77 (s, 1H), 8.29 (s, 1H), 7.50 (d, J=5 Hz, 1H), 6.92 (t, J=5 Hz, 1H), 6.76 (s, 1H), 6.53-6.46 (m, 1H), 5.83 (s, 1H), 5.01-4.94 (m, 1H), 4.51 (s, 2H), 4.20-4.13 (m, 1H), 4.01-3.94 (m, 2H), 3.55-3.48 (m, 2H), 3.45-3.38 (m, 1H), 3.10-3.01 (m, 1H), 2.93-2.75 (m, 4H), 2.61-2.55 (m, 2H), 2.55-2.52 (m, 1H), 2.48-2.43 (m, 1H), 2.17-1.96 (m, 5H), 1.94-1.80 (m, 5H), 1.76-1.66 (m, 2H), 1.62-1.53 (m, 1H), 1.21 (m, 9H). LC-MS (ESI): m/z [M+H]⁺=827.4

### Example 5: (1S,3R)-3-(3-((2-(1-(1-(4-((R)-2,6-dioxopiperidin-3-yl)-3,5-difluorophenyl) azetidin-3-yl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

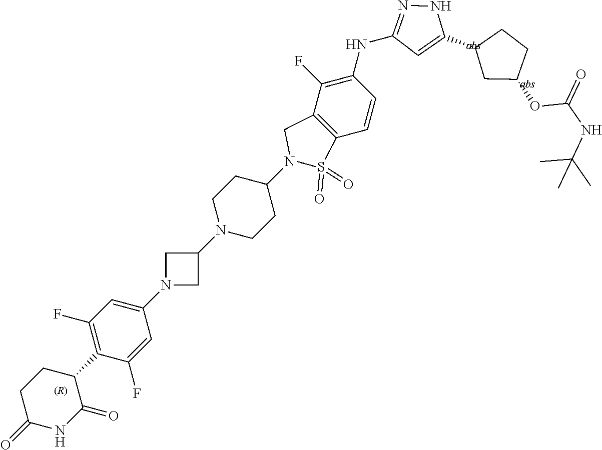

The titled compound was synthesized in the procedures similar to Example 1. ¹H NMR (500 MHz, DMSO-d₆) δ 11.98 (s, 1H), 10.86 (s, 1H), 8.77 (s, 1H), 8.29 (t, J=5 Hz, 1H), 7.51 (d, J=10 Hz, 1H), 6.76 (s, 1H), 6.13 (d, J=10 Hz, 2H), 5.83 (s, 1H), 5.01-4.94 (m, 1H), 4.51 (s, 2H), 4.07-4.01 (m, 1H), 3.93 (t, J=10 Hz, 2H), 3.66-3.60 (m, 2H), 3.51-3.44 (m, 1H), 3.30-3.24 (m, 1H), 3.10-3.01 (m, 1H), 2.89-2.75 (m, 3H), 2.47-2.42 (m, 1H), 2.13-1.98 (m, 4H), 1.98-1.80 (m, 6H), 1.76-1.66 (m, 2H), 1.62-1.53 (m, 1H), 1.21 (s, 10H). LC-MS (ESI): m/z [M+H]⁺=813.4

### Example 6: (1S,3R)-3-(3-((2-((1-(1-(4-((R)-2,6-dioxopiperidin-3-yl)-3,5-difluorophenyl) azetidin-3-yl)piperidin-4-yl)methyl)-4-fluoro-1,1-dioxido-2,3-dihydrobenzo[d]isothiazol-5-yl)amino)-1H-pyrazol-5-yl)cyclopentyl tert-butylcarbamate

### Step 1: (1S,3R)-3-(3-((4-fluoro-1,1-dioxido-2-(piperidin-4-ylmethyl)-2,3-dihydrobenzo [d]isothiazol-5-yl)amino)-1H-pyrazol-5-yl)cyclopentyl tert-butylcarbamate

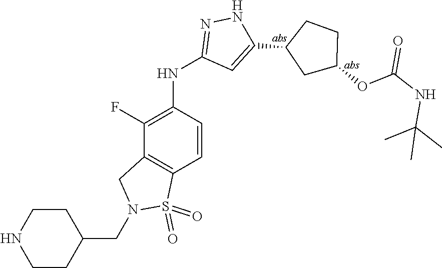 [SMILES: CC(C)(C)NC(=O)O[C@H]1CC[C@@H](c2cc(Nc3ccc4c(c3F)CN(CC3CCNCC3)S4(=O)=O)n[nH]2)C1]

The titled compound was synthesized in the procedures similar to Example 1, step 9-10. LC-MS (ESI): m/z [M+H]⁺=549.5

### Step 2: (1S,3R)-3-(3-((2-((1-(1-(4-((R)-2,6-dioxopiperidin-3-yl)-3,5-difluorophenyl) azetidin-3-yl)piperidin-4-yl)methyl)-4-fluoro-1,1-dioxido-2,3-dihydrobenzo[d]isothiazol-5-yl)amino)-1H-pyrazol-5-yl)cyclopentyl tert-butylcarbamate

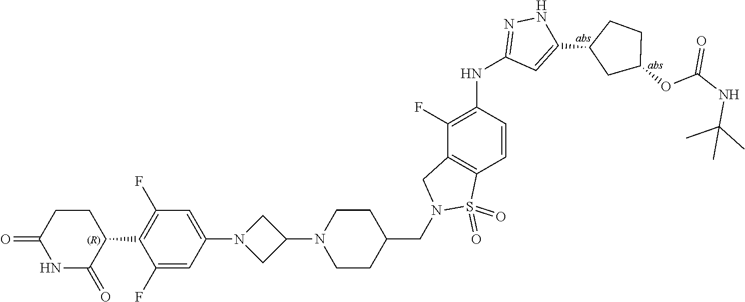

The titled compound was synthesized in the procedures similar to Example 1. ¹H NMR (500 MHz, DMSO-d₆) δ 11.99 (s, 1H), 10.85 (s, 1H), 8.78 (s, 1H), 8.30 (s, 1H), 7.53 (d, J=10 Hz, 1H), 6.76 (s, 1H), 6.12 (d, J=10 Hz, 1H), 5.83 (s, 1H), 5.03-4.94 (m, 1H), 4.49 (s, 2H), 4.07-4.01 (m, 1H), 3.92 (t, J=10 Hz, 2H), 3.66-3.58 (m, 2H), 3.26-3.19 (m, 1H), 3.10-2.98 (m, 3H), 2.84-2.72 (m, 3H), 2.48-2.43 (m, 2H), 2.13-1.98 (m, 2H), 1.9-1.8 (m, 4H), 1.80-1.64 (m, 5H), 1.63-1.53 (m, 1H), 1.25-1.13 (m, 10H). LC-MS (ESI): m/z [M+H]⁺=827.2.

### Example 7: (1S,3R)-3-(3-((2-(1′-(1-(4-((R)-2,6-dioxopiperidin-3-yl)-3,5-difluorophenyl)azetidin-3-yl)-[1,4′-bipiperidin]-4-yl)-4-fluoro-1,1-dioxido-2,3-dihydrobenzo[d]isothiazol-5-yl)amino)-1H-pyrazol-5-yl)cyclopentyl tert-butylcarbamate

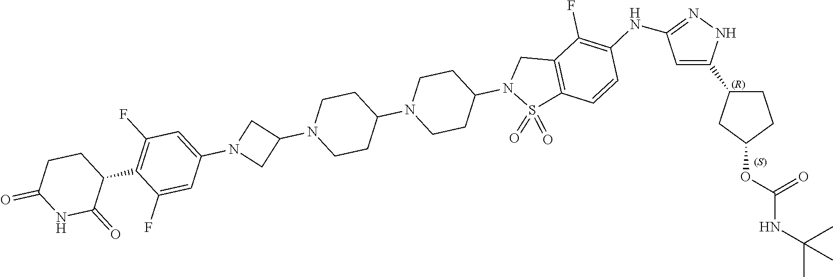

### Step 1: (1S,3R)-3-(3-((2-(1′-(1-(4-((R)-2,6-dioxopiperidin-3-yl)-3,5-difluorophenyl-azetidin-3-yl)-[1,4′-bipiperidin]-4-yl)-4-fluoro-1,1-dioxido-2,3-dihydrobenzo[d]isothia-zol-5-yl)amino)-1H-pyrazol-5-yl)cyclopentyl tert-butylcarbamate

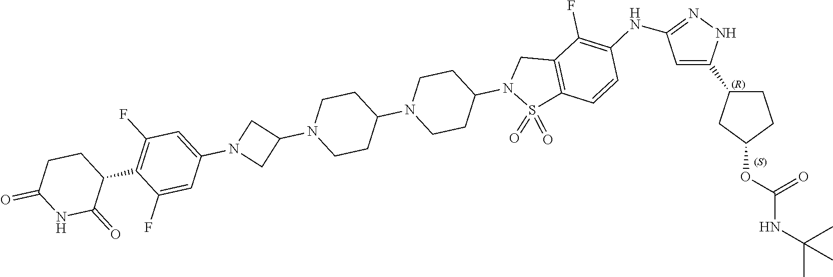

To a solution of (1S,3R)-3-(3-((2-([1,4′-bipiperidin]-4-yl)-4-fluoro-1,1-dioxido-2,3-dihydrobenzo[d]isothiazol-5-yl)amino)-1H-pyrazol-5-yl)cyclopentyl tert-butyl carbamate (30 mg, 0.049 mmol), (R)-3-(2,6-difluoro-4-(3-oxoazetidin-1-yl)phenyl)piperidine-2,6-dione (24 mg, 0.067 mmol) and Acetic acid (3 mg, 0.049 mmol) in DMA (1.5 mL) was stirred for 30 min, then STAB (22 mg, 0.169 mmol) was added. the reaction mixture was stirred for 16 hours at room temperature. The resulting mixture was concentrated under reduce pressure, the residue was purified by column chromatography on silica gel, eluting with DCM/MeOH (10:1) to afford the product (20 mg, 46%). The residue 20 mg was purified by prep HPLC (Waters XSelect C18: RD-CO-094 column, eluting with a gradient of acetonitrile/water containing 0.1% FA, 20%-40%) to afford the product (10 mg, 23%). LC-MS (ESI): m/z [M+H]⁺=896.62.

¹H NMR (500 MHz, DMSO) δ 11.98 (s, 1H), 10.85 (s, 1H), 8.76 (s, 1H), 8.28 (s, 1H), 7.50 (d, J=8.6 Hz, 1H), 6.76 (s, 1H), 6.11 (d, J=11.1 Hz, 2H), 5.83 (s, 1H), 4.98 (s, 1H), 4.50 (s, 2H), 4.03 (dd, J=12.6, 5.0 Hz, 1H), 3.91 (t, J=7.2 Hz, 2H), 3.60 (t, J=5.3 Hz, 2H), 3.45-3.36 (m, 3H), 3.23-3.18 (m, 1H), 3.10-3.02 (m, 1H), 2.93 (d, J=10.9 Hz, 2H), 2.83 (d, J=10.6 Hz, 2H), 2.76 (dd, J=17.4, 5.0 Hz, 1H), 2.3-2.21 (m, 3H), 2.12-2.00 (m, 2H), 1.95-1.87 (m, 4H), 1.85-1.70 (m, 8H), 1.58 (s, 1H), 1.48-1.37 (m, 2H), 1.21 (s, 9H).

### Example 8a and 8b: (1S,3R)-3-(3-((2-(1-((1-(4-((R)-2,6-dioxopiperidin-3-yl)-3,5-difluorophenyl)-4-hydroxypiperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate and (1S,3R)-3-(3-((2-(1-((1-(4-((R)-2,6-dioxopiperidin-3-yl)-3,5-difluorophenyl)-4-hydroxypiperidin-4-yl)methyl)piperidin-4-yl)-4-fluoro-1,1-dioxido-2,3-dihydrobenzo [d]isothiazol-5-yl)amino)-1H-pyrazol-5-yl)cyclopentyl tert-butylcarbamate

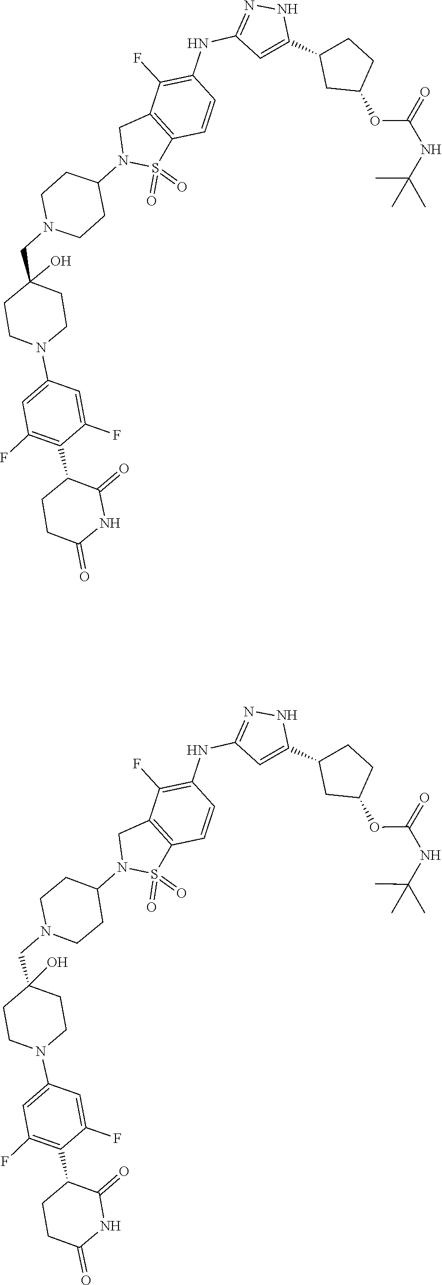

### Step 1: (R)-3-(2,6-difluoro-4-(1-oxa-6-azaspiro[2.5]octan-6-yl)phenyl)piperidine-2,6-dione

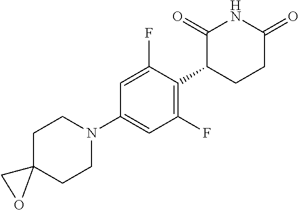 [SMILES: O=C1CC[C@H](c2c(F)cc(N3CCC4(CC3)CO4)cc2F)C(=O)N1]

To a solution of (R)-3-(2,6-difluoro-4-(4-oxopiperidin-1-yl)phenyl)piperidine-2,6-dione (500 mg, 1.55 mmol) in DMSO (10 mL) was added trimethylsulfoxonium iodide (682 mg, 3.10 mmol) and t-BuOK (347.2 mg, 3.10 mmol). The reaction mixture was stirred for 3 hours at room temperature. The resulting mixture was diluted with EA, and washed with water and brine. The combined organic phase was dried over anhydrous Na₂SO₄, then filtered and concentrated under reduced pressure. The residue was purified by silica gel column chromatography, eluting with PE/EA (1:3) to afford the product (280 mg, 53.7% yield). LC-MS (ESI): m/z [M+H]⁺=337.2.

### Step 2: (1S,3R)-3-(3-((2-(1-((1-(4-((R)-2,6-dioxopiperidin-3-yl)-3,5-difluorophenyl)-4-hydroxypiperidin-4-yl)methyl)piperidin-4-yl)-4-fluoro-1,1-dioxido-2,3-dihydrobenzo [d]isothiazol-5-yl)amino)-1H-pyrazol-5-yl)cyclopentyl tert-butylcarbamate and (1S,3R)-3-(3-((2-(1-((1-(4-((R)-2,6-dioxopiperidin-3-yl)-3,5-difluorophenyl)-4-hydroxypiperidin-4-yl)methyl)piperidin-4-yl)-4-fluoro-1,1-dioxido-2,3-dihydrobenzo [d]isothiazol-5-yl)amino)-1H-pyrazol-5-yl)cyclopentyl tert-butylcarbamate

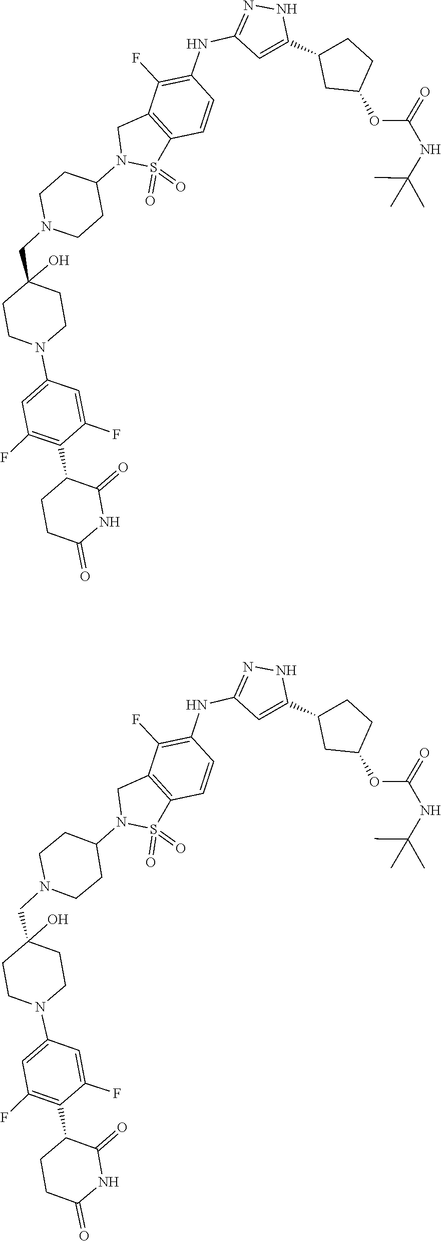

To a solution of (1S,3R)-3-(3-((4-fluoro-1,1-dioxido-2-(piperidin-4-yl)-2,3-dihydrobenzo[d]isothiazol-5-yl)amino)-1H-pyrazol-5-yl)cyclopentyl tert-butylcarbamate (100 mg, 0.187 mmol) and (R)-3-(2,6-difluoro-4-(1-oxa-6-azaspiro[2.5]octan-6-yl)phenyl) piperidine-2,6-dione (75.6 mg, 0.224 mmol) in MeOH (5 mL), the reaction mixture was stirred for 16 hours at 50° C. under nitrogen. The resulting mixture was concentrated under reduce pressure, The residue was purified by silica gel column chromatography, eluting with DCM/MeOH (10:1) to afford the product in a racemic form, which was further separated by Chiral Prep-HPLC to give:

Enantiomer 1 (example 8a, 100% ee); Retention time: 10.667 min. ¹H NMR (500 MHz, DMSO) δ 11.98 (s, 1H), 10.86 (s, 1H), 8.77 (s, 1H), 8.30 (s, 1H), 7.50 (d, J=8.6 Hz, 1H), 6.76 (s, 1H), 6.62 (d, J=13.0 Hz, 2H), 5.83 (s, 1H), 4.98 (s, 1H), 4.51 (s, 2H), 4.19 (s, 1H), 4.04 (dd, J=12.4, 4.9 Hz, 1H), 3.45 (m, 3H), 3.07 (m, 5H), 2.84-2.73 (m, 2H), 2.45 (m, 1H), 2.30 (m, 4H), 2.15-1.79 (m, 9H), 1.70 (m, 2H), 1.55 (m, 5H), 1.21 (s, 9H). LC-MS (ESI): m/z [M+H]⁺=871.3

Enantiomer 2 (example 8b, 100% ee); Retention time: 13.777 min. ¹H NMR (500 MHz, DMSO) δ 11.98 (s, 1H), 10.86 (s, 1H), 8.77 (s, 1H), 8.30 (s, 1H), 7.50 (d, J=8.6 Hz, 1H), 6.76 (s, 1H), 6.62 (d, J=13.0 Hz, 2H), 5.83 (s, 1H), 4.98 (s, 1H), 4.51 (s, 2H), 4.19 (s, 1H), 4.04 (dd, J=12.4, 4.9 Hz, 1H), 3.45 (m, 3H), 3.07 (m, 5H), 2.84-2.73 (m, 2H), 2.45 (m, 1H), 2.30 (m, 4H), 2.15-1.79 (m, 9H), 1.70 (m, 2H), 1.55 (m, 5H), 1.21 (s, 9H). LC-MS (ESI): m/z [M+H]⁺=871.3

Chiral analytical method: Column: CHIRALPAK IE 4.6 mm*150 mmL, 5 μm; Mobile phase: A for MtBE (0.1% FA)(%) and B for MeOH:DCM=50:50 (v/v); Gradient: Mobile Phase A: Mobile Phase B=20:80 (v/v); HPLC Equipment: Agilent 1260 Infinity II (ARD_LC-808); Column temperature: 25° C.

Chiral Prep-HPLC Condition: Column: CHIRALPAK®IE, 250*20.0 mml, 5 μm; Mobile phase: A for MtBE and B for DCM:MeOH=1:1; Gradient: Mobile Phase A: Mobile Phase B=20:80 (v/v); Flow Rate: 18 mL/min, Wave Length: UV 250 nm and 290 nm, Prep-HPLC Equipment: Prep-HPLC Gilson (ARD_LC-815), Back pressure: 100 bar; Column temperature: 25° C.

### Example 9 and 10: (1S,3R)-3-(3-((2-(cis-4-(4-(4-((R)-2,6-dioxopiperidin-3-yl)-3,5-difluorophenyl)piperazin-1-yl)cyclohexyl)-4-fluoro-1,1-dioxido-2,3-dihydrobenzo[d]isothiazol-5-yl)amino)-1H-pyrazol-5-yl)cyclopentyl tert-butylcarbamate and (1S,3R)-3-(3-((2-(trans-4-(4-(4-((R)-2,6-dioxopiperidin-3-yl)-3,5-difluorophenyl)piperazin-1-yl) cyclohexyl)-4-fluoro-1,1-dioxido-2,3-dihydrobenzo[d]isothiazol-5-yl)amino)-1H-pyrazol-5-yl)cyclopentyl tert-butylcarbamate

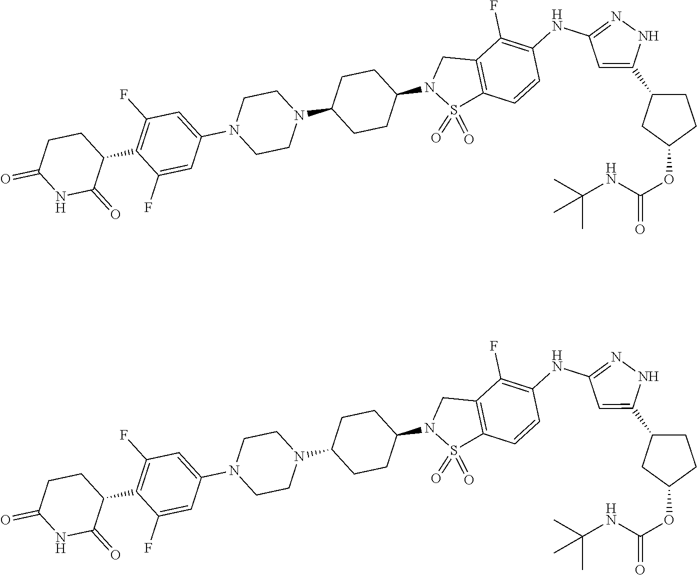 [SMILES: CC(C)(C)NC(=O)O[C@H]1CC[C@@H](c2cc(Nc3ccc4c(c3F)CN([C@H]3CC[C@@H](N5CCN(c6cc(F)c([C@H]7CCC(=O)NC7=O)c(F)c6)CC5)CC3)S4(=O)=O)n[nH]2)C1 ; CC(C)(C)NC(=O)O[C@H]1CC[C@@H](c2cc(Nc3ccc4c(c3F)CN([C@H]3CC[C@H](N5CCN(c6cc(F)c([C@H]7CCC(=O)NC7=O)c(F)c6)CC5)CC3)S4(=O)=O)n[nH]2)C1]

The titled compound was synthesized in the procedures similar to Example 2 in a racemic form, which was further separated by Chiral Prep-HPLC to give:

Enantiomer 1 (example 9, 100% ee); Retention time: 4.878 min. ¹H NMR (500 MHz, DMSO) δ=11.98 (s, 1H), 10.87 (s, 1H), 8.76 (s, 1H), 8.29 (s, 1H), 7.50 (d, J=8.7, 1H), 6.76 (s, 1H), 6.63 (d, J=12.7, 2H), 5.83 (s, 1H), 4.98 (s, 1H), 4.50 (s, 2H), 4.05 (dd, J=12.7, 4.9, 1H), 3.46 (m, 1H), 3.17 (m, 4H), 3.07 (m, 1H), 2.77 (m, 2H), 2.61 (m, 4H), 2.46-2.45 (m, 1H), 2.32 (m, 1H), 2.14-2.07 (m, 1H), 2.05-1.98 (m, 3H), 1.98-1.87 (m, 4H), 1.68 (m, 4H), 1.58 (m, 1H), 1.42 (m, 2H), 1.21 (s, 9H). LC-MS (ESI): m/z [M+H]⁺=841.64.

Enantiomer 2 (example 10, 100% ee); Retention time: 7.359 min. ¹H NMR (500 MHz, DMSO) δ=11.98 (s, 1H), 10.87 (s, 1H), 8.77 (s, 1H), 8.29 (s, 1H), 7.50 (d, J=8.6, 1H), 6.76 (s, 1H), 6.63 (d, J=12.6, 2H), 5.83 (s, 1H), 4.97 (s, 1H), 4.45 (s, 2H), 4.05 (dd, J=12.4, 4.8, 1H), 3.48 (m, 2H), 3.19 (m, 4H), 3.06 (m, 1H), 2.82-2.71 (m, 2H), 2.57-2.55 (m, 4H), 2.46-2.43 (m, 1H), 2.28 (m, 1H), 2.14 (m, 2H), 2.02 (m, 1H), 1.98-1.93 (m, 1H), 1.93-1.81 (m, 3H), 1.76-1.69 (m, 2H), 1.64 (m, 2H), 1.60 (m, 3H), 1.23-1.16 (s, 9H). LC-MS (ESI): m/z [M+H]⁺=841.62;

Chiral analytical method: Column: CHIRALPAK IE 4.6 mm*150 mmL, 5 μm; Mobile phase: A for MtBE (0.1% FA)(%) and B for MeOH:DCM=50:50 (v/v); Gradient: Mobile Phase A: Mobile Phase B=20:80 (v/v); HPLC Equipment: Agilent 1260 Infinity II (ARD_LC-808); Column temperature: 25° C.

Chiral Prep-HPLC Condition: Column: CHIRALPAK®IE, 250*20.0 mml, 5 μm; Mobile phase: A for MtBE and B for DCM:MeOH=1:1; Gradient: Mobile Phase A: Mobile Phase B=20:80 (v/v); Flow Rate: 18 mL/min, Wave Length: UV 250 nm and 290 nm, Prep-HPLC Equipment: Prep-HPLC Gilson (ARD_LC-815), Back pressure: 100 bar; Column temperature: 25° C.

### Example 11: (1S,3R)-3-(3-((2-(1-((1-(3-((R)-2,6-dioxopiperidin-3-yl)-2,4-difluorophenyl) piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

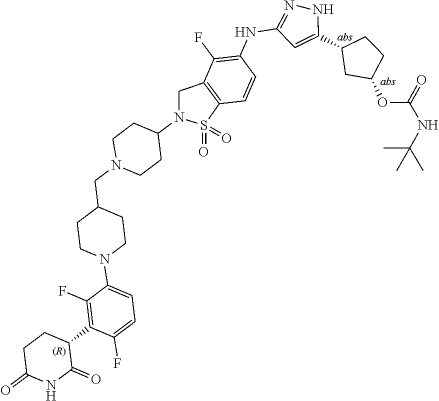

The titled compound was synthesized in the procedures similar to Example 1. ¹H NMR (500 MHz, DMSO-d₆) δ 11.98 (s, 1H), 10.94 (s, 1H), 8.77 (s, 1H), 8.30 (t, J=10 Hz, 1H), 7.51 (d, J=10 Hz, 1H), 7.11-6.92 (m, 2H), 6.79-6.74 (m, 1H), 5.83 (s, 1H), 5.03-4.95 (m, 1H), 4.51 (s, 2H), 4.24-4.17 (m, 1H), 3.47-3.39 (m, 1H), 3.26-3.18 (m, 2H), 3.10-3.01 (m, 1H), 2.94-2.85 (m, 2H), 2.84-2.76 (m, 1H), 2.67-2.59 (m, 2H), 2.46-2.43 (m, 1H), 2.22-2.08 (m, 4H), 2.06-1.96 (m, 4H), 1.94-1.76 (m, 7H), 1.75-1.67 (m, 2H), 1.66-1.54 (m, 2H), 1.30-1.22 (m, 2H), 1.21 (s, 9H). LC-MS (ESI): m/z [M+H]⁺=855.

### Example 12: (1S,3R)-3-(3-((2-(1-(1-(4-((R)-2,6-dioxopiperidin-3-yl)-3,5-difluorophenyl)piperidine-4-carbonyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

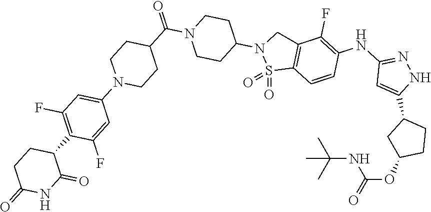

### Step 1: tert-butyl 1-(4-(2,6-bis(benzyloxy)pyridin-3-yl)-3,5-difluorophenyl)piperidine-4-carboxylate

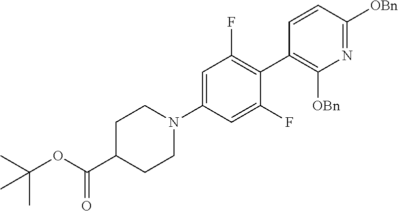 [SMILES: CC(C)(C)OC(=O)C1CCN(c2cc(F)c(-c3ccc(OCc4ccccc4)nc3OCc3ccccc3)c(F)c2)CC1]

To a solution of 2,6-bis(benzyloxy)-3-(4-bromo-2,6-difluorophenyl)pyridine (400 mg, 0.83 mmol) and tert-butyl piperidine-4-carboxylate (154 mg, 0.83 mmol) in dioxane (15 mL) was added Pd₂dba₃ (38 mg, 0.042 mmol), RuPhos (39 mg, 0.083 mmol) and Cs₂CO₃ (810 mg, 2.5 mmol), the reaction mixture was stirred for 16 hours at 100° C. under nitrogen. The resulting mixture was cooled to room temperature, filtered, the filtrate was concentrated under reduce pressure. The residue was purified by silica gel column chromatography, eluting with PE/EA (3:1) to afford the product (445 mg, 91% yield). LC-MS (ESI): m/z [M+H]⁺=587.4.

### Step 2: tert-butyl 1-(4-(2,6-dioxopiperidin-3-yl)-3,5-difluorophenyl)piperidine-4-carboxylate

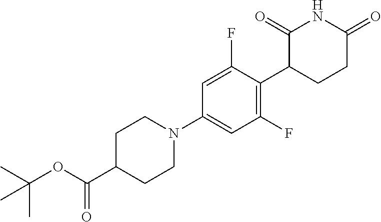 [SMILES: CC(C)(C)OC(=O)C1CCN(c2cc(F)c(C3CCC(=O)NC3=O)c(F)c2)CC1]

To a solution of tert-butyl 1-(4-(2,6-bis(benzyloxy)pyridin-3-yl)-2,6-difluorophenyl) piperidine-4-carboxylate (445 mg, 0.76 mmol) in dioxane (20 mL) was added Pd/C (10% on carbon, wet, 100 mg) and Pd(OH)₂/C (10% on carbon, wet, 100 mg). The suspension was degassed and purged with hydrogen 3 times. The resulting mixture was stirred at 50° C. under a hydrogen balloon for 16 h. The mixture was cooled to room temperature, filtered, and the filter cake was washed with EA (20 mL×3). The filtrate was concentrated under reduced pressure to afford the product (310 mg, 100% yield). LC-MS (ESI): m/z [M+H]⁺=409.5.

### Step 3: 1-(4-(2,6-dioxopiperidin-3-yl)-3,5-difluorophenyl)piperidine-4-carboxylic acid

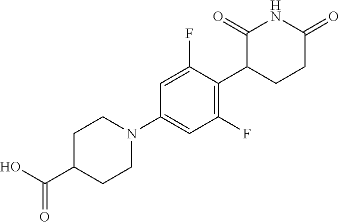 [SMILES: O=C1CCC(c2c(F)cc(N3CCC(C(=O)O)CC3)cc2F)C(=O)N1]

To a solution of tert-butyl 1-(4-(2,6-dioxopiperidin-3-yl)-3,5-difluorophenyl)piperidine-4-carboxylate (310 mg, 0.76 mmol) in dichloromethane (10 mL) was added trifluoroacetic acid (2 mL). The reaction mixture was stirred for 16 hours at room temperature. The resulting mixture was concentrated, dried over in vacuum, to afford the product (268 mg, 100% yield). LC-MS (ESI): m/z [M+H]⁺=353.3.

### Step 4: (1S,3R)-3-(3-((2-(1-(1-(4-((R)-2,6-dioxopiperidin-3-yl)-3,5-difluorophenyl) piperidine-4-carbonyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

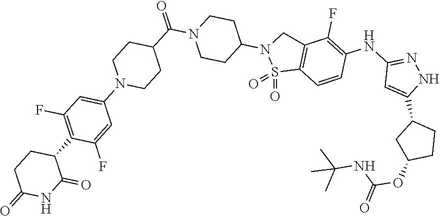 [SMILES: CC(C)(C)NC(=O)O[C@H]1CC[C@@H](c2cc(Nc3ccc4c(c3F)CN(C3CCN(C(=O)C5CCN(c6cc(F)c([C@H]7CCC(=O)NC7=O)c(F)c6)CC5)CC3)S4(=O)=O)n[nH]2)C1]

To a solution of (1S,3R)-3-(3-((4-fluoro-1,1-dioxido-2-(piperidin-4-yl)-2,3-dihydrobenzo [d]isothiazol-5-yl)amino)-1H-pyrazol-5-yl)cyclopentyl tert-butylcarbamate (30 mg, 0.056 mmol) and 1-(4-(2,6-dioxopiperidin-3-yl)-3,5-difluorophenyl)piperidine-4-carboxylic acid (24 mg, 0.067 mmol) in DMF (1.5 mL) was added HATU (32 mg, 0.084 mmol) and DIPEA (22 mg, 0.169 mmol), the reaction mixture was stirred for 16 hours at room temperature. The resulting mixture was concentrated under reduce pressure, the residue was purified by column chromatography on silica gel, eluting with DCM/MeOH (10:1) to afford the product (35 mg, 72%) in a racemic form, which was further separated by Chiral Prep-HPLC to give:

Example 12 (100% ee); Retention time: 8.621 min. ¹H NMR (500 MHz, DMSO) δ=11.98 (s, 1H), 10.87 (s, 1H), 8.78 (s, 1H), 8.31 (t, J=7.5, 1H), 7.52 (d, J=8.6, 1H), 6.76 (s, 1H), 6.63 (d, J=12.8, 2H), 5.83 (s, 1H), 4.98 (s, 1H), 4.52 (s, 2H), 4.39 (d, J=11.8, 1H), 4.11-4.01 (m, 2H), 3.78 (m, 3H), 3.24 (m, 1H), 3.09-3.01 (m, 1H), 2.88 (m, 3H), 2.82-2.74 (m, 2H), 2.45 (m, 1H), 2.14-1.79 (m, 8H), 1.76-1.53 (m, 8H), 1.20 (s, 9H). LC-MS (ESI): m/z [M+H]⁺=869.62.

Chiral analytical method: Column: CHIRALPAK IE 4.6*150 mm 5 μm; Mobile phase: A for MtBE (0.1% FA)(%) and B for MeOH:DCM=50:50 (v/v); Gradient: Mobile Phase A: Mobile Phase B=30:70 (v/v); HPLC Equipment: Agilent 1260 Infinity II (ARD_LC-808); Column temperature: 35° C.

Chiral Prep-HPLC Condition: CHIRALPAK IE 250*20.0 mml, 5 μm; Mobile phase: A for MtBE (0.2% FA) and B for MeOH:DCM=50:50; Gradient: Mobile Phase A: Mobile Phase B=30:70 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 13: (1S,3R)-3-(3-((2-((3R,4R)-1-((1-(4-((R)-2,6-dioxopiperidin-3-yl)-3,5-difluorophenyl)piperidin-4-yl)methyl)-3-fluoropiperidin-4-yl)-4-fluoro-1,1-dioxido-2,3-dihydrobenzo[d]isothiazol-5-yl)amino)-1H-pyrazol-5-yl)cyclopentyl tert-butylcarbamate

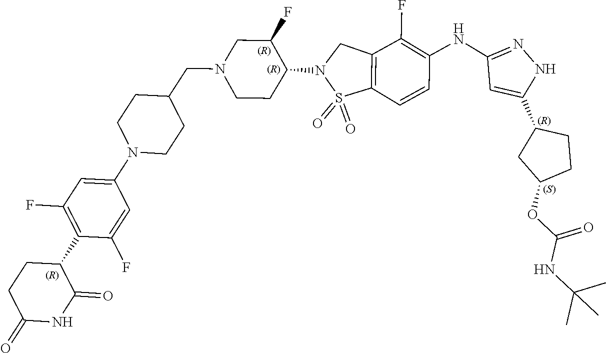

### Step 1: (1S,3R)-3-(3-((4-fluoro-1,1-dioxido-2,3-dihydrobenzo[d]isothiazol-5-yl)amino)-1H-pyrazol-5-yl cyclopentyl tert-butylcarbamate

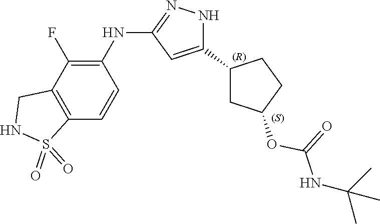

To a mixture of 5-bromo-4-fluoro-2,3-dihydrobenzo[d]isothiazole 1,1-dioxide (1.8 g, 6.79 mmol) and (1S,3R)-3-(3-amino-1H-pyrazol-5-yl)cyclopentyl tert-butylcarbamate (1.8 g, 6.79 mmol) in t-BuOH (300 mL) was added Brettphos Pd G3 (616 mg, 0.68 mmol) and K₂CO₃ (2.8 g, 20.37 mmol). The reaction mixture was stirred under nitrogen atmosphere at 110° C. for 16 h. The mixture was filtered, and the filtrate was concentrated under reduced pressure. The residue was purified by silica gel column chromatography, eluting with PE/EA (1:9) to afford the product (2.3 g, 75% yield). LC-MS (ESI): m/z [M+H]⁺=452.3.

### Step 2: tert-butyl (3R,4R)-4-(5-((5-((1R,3S)-3-((tert-butylcarbamoyl)oxy)cyclopentyl)-1H-pyrazol-3-yl)amino)-4-fluoro-1,1-dioxidobenzo[d]isothiazol-2(3H)-yl)-3-fluoropiperi-dine-1-carboxylate

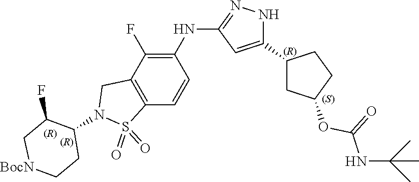

To a mixture of (1S,3R)-3-(3-((4-fluoro-1,1-dioxido-2,3-dihydrobenzo[d]isothiazol-5-yl)amino)-1H-pyrazol-5-yl)cyclopentyl tert-butylcarbamate (100 mg, 0.22 mmol) and tert-butyl (3R,4S)-3-fluoro-4-hydroxypiperidine-1-carboxylate (53 mg, 0.24 mmol) in toluene (2 mL) was added (160 mg, 0.66 mmol). The reaction mixture was stirred under nitrogen atmosphere at 100° C. for 16 h. The mixture was concentrated under reduced pressure. The residue was purified by silica gel column chromatography, eluting with PE/EA (1:9) to afford the product (80 mg, 69% yield). LC-MS (ESI): m/z [M+H]⁺=653.3.

### Step 3: (1S,3R)-3-(3-((4-fluoro-2-((3R,4R)-3-fluoropiperidin-4-yl)-1,1-dioxido-2,3-dihydrobenzo[d]isothiazol-5-yl)amino)-1H-pyrazol-5-yl)cyclopentyl tert-butylcarbamate

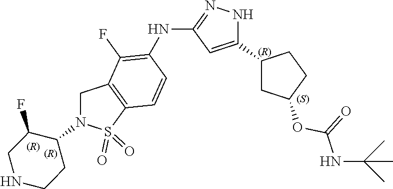

To a solution of tert-butyl (3R,4R)-4-(5-((5-((1R,3S)-3-((tert-butylcarbamoyl)oxy)cyclo-pentyl)-1H-pyrazol-3-yl)amino)-4-fluoro-1,1-dioxidobenzo[d]isothiazol-2(3H)-yl)-3-fluoro-piperi-dine-1-carboxylate (80 mg, 0.12 mmol) in dichloromethane (5 mL) was added trifluoroacetic acid (2 mL). The reaction mixture was stirred for 1 hours at room temperature. The resulting mixture was concentrated, dried over in vacuum, to afford the product (67 mg, 100% yield). LC-MS (ESI): m/z [M+H]⁺=553.3.

### Step 4: (1S,3R)-3-(3-((2-((3R,4R)-1-((1-(4-(2,4-dioxotetrahydropyrimidin-1(2H)-yl)phenyl)piperidin-4-yl)methyl)-3-fluoropiperidin-4-yl)-4-fluoro-1,1-dioxido-2,3-dihyd-robenzo[d]isothiazol-5-yl)amino)-1H-pyrazol-5-yl)cyclopentyl tert-butylcarbamate

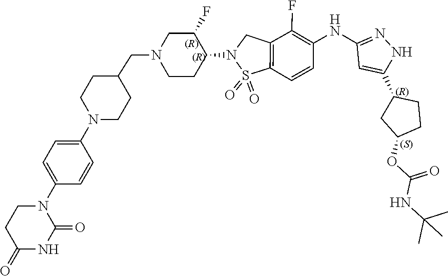

To a solution of (1S,3R)-3-(3-((4-fluoro-2-((3R,4R)-3-fluoropiperidin-4-yl)-1,1-dioxido-2,3-dihydrobenzo[d]isothiazol-5-yl)amino)-1H-pyrazol-5-yl)cyclopentyl tert-butyl carbamate (30 mg, 0.046 mmol), 1-(4-(2,4-dioxotetrahydropyrimidin-1(2H)-yl)phenyl)pip-eridine-4-carbaldehyde (21 mg, 0.069 mmol) and Sodium acetate (30 mg, 0.092 mmol) in DMA (1.5 mL) was stirred for 30 min, then STAB (22 mg, 0.169 mmol) was added. the reaction mixture was stirred for 16 hours at room temperature. The resulting mixture was concentrated under reduce pressure, the residue was purified by column chromatography on silica gel, eluting with DCM/MeOH (10:1) to afford the product (25 mg, 65%). The residue 25 mg was purified by prep HPLC (Waters XSelect C18: RD-CO-094 column, eluting with a gradient of acetonitrile/water containing 0.1% FA, 20%-40%) to afford the product (10 mg, 32.5%). LC-MS (ESI): m/z [M+H]⁺=838.5.

¹H NMR (500 MHz, DMSO) δ 11.97 (s, 1H), 10.86 (s, 1H), 9.66 (d, J=78.6 Hz, 1H), 8.82 (s, 1H), 8.31 (d, J=7.5 Hz, 1H), 7.58 (t, 1H), 6.73 (s, 1H), 6.66 (d, J=12.9 Hz, 2H), 5.85 (s, 1H), 5.19 (d, J=49.3 Hz, 1H), 4.98 (s, 1H), 4.64 (dd, 2H), 4.06 (dd, J=5.0 Hz, 1H), 3.95 (s, 1H), 3.82 (d, J=11.8 Hz, 2H), 3.60 (s, 1H), 3.18 (d, J=12.9 Hz, 1H), 3.12-3.03 (m, 2H), 2.83-2.76 (m, 3H), 2.52 (m, J=3.5 Hz, 1H), 2.50 (m, J=1.7 Hz, 4H), 2.38-2.28 (m, 1H), 2.19 (s, 1H), 2.12-2.03 (m, 2H), 1.99-1.88 (m, 2H), 1.85-1.68 (m, 4H), 1.59 (s, 1H), 1.25 (d, J=11.7 Hz, 2H), 1.21 (s, 9H).

### Example 14: (1S,3R)-3-(3-((2-((3S,4R)-1-((1-(4-(2,4-dioxotetrahydropyrimidin-1(2H)-yl)phenyl)piperidin-4-yl)methyl)-3-fluoropiperidin-4-yl)-4-fluoro-1,1-dioxido-2,3-dihydro-benzo[d]isothiazol-5-yl)amino)-1H-pyrazol-5-yl)cyclopentyl tert-butylcarbamate

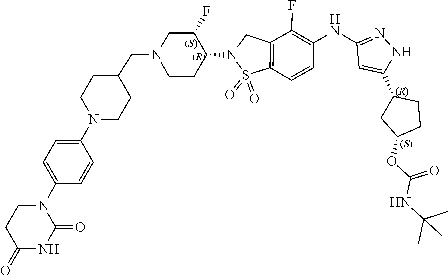

The titled compound was synthesized in the procedures similar to Example 13. ¹H NMR (500 MHz, DMSO) δ 11.99 (s, 1H), 10.85 (s, 1H), 8.79 (s, 1H), 8.31 (s, 1H), 7.55 (d, J=8.7 Hz, 1H), 6.75 (s, 1H), 6.61 (d, J=12.9 Hz, 2H), 5.84 (s, 1H), 5.04-4.87 (m, 2H), 4.60 (dd, J=48.1, 14.0 Hz, 2H), 4.04 (dd, J=12.5, 4.9 Hz, 1H), 3.80-3.62 (m, 3H), 3.18-3.00 (m, 2H), 2.95 (d, J=9.1 Hz, 1H), 2.84-2.68 (m, 3H), 2.34 (d, J=13.7 Hz, 1H), 2.28-2.15 (m, 4H), 2.11-1.86 (m, 4H), 1.81-1.67 (m, 6H), 1.59 (s, 1H), 1.27-1.08 (m, 11H). LC-MS (ESI): m/z [M+H]⁺=553.3.

### Example 15a and 15b and 15c and 15d: (1S,3R)-3-(3-((2-((3R,4S)-1-((1-(4-((R)-2,6-dioxopiperidin-3-yl)-3,5-difluorophenyl)piperidin-4-yl)methyl)-3-methylpiperidin-4-yl)-4-fluoro-1,1-dioxido-2,3-dihydrobenzo[d]isothiazol-5-yl)amino)-1H-pyrazol-5-yl)cyclopentyl tert-butylcarbamate and

### (1S,3R)-3-(3-((2-((3S,4S)-1-((1-(4-((R)-2,6-dioxopiperidin-3-yl)-3,5-difluorophenyl)piperidin-4-yl)methyl)-3-methylpiperidin-4-yl)-4-fluoro-1,1-dioxido-2,3-dihydrobenzo[d]isothiazol-5-yl)amino)-1H-pyrazol-5-yl)cyclopentyl tert-butylcarbamate and

### (1S,3R)-3-(3-((2-((3R,4R)-1-((1-(4-((R)-2,6-dioxopiperidin-3-yl)-3,5-difluorophenyl)piperidin-4-yl)methyl)-3-methylpiperidin-4-yl)-4-fluoro-1,1-dioxido-2,3-dihydrobenzo[d]isothiazol-5-yl)amino)-1H-pyrazol-5-yl)cyclopentyl tert-butylcarbamate and

### (1S,3R)-3-(3-((2-((3S,4R)-1-((1-(4-((R)-2,6-dioxopiperidin-3-yl)-3,5-difluorophenyl)piperidin-4-yl)methyl)-3-methylpiperidin-4-yl)-4-fluoro-1,1-dioxido-2,3-dihydrobenzo[d]isothiazol-5-yl)amino)-1H-pyrazol-5-yl)cyclopentyl tert-butylcarbamate

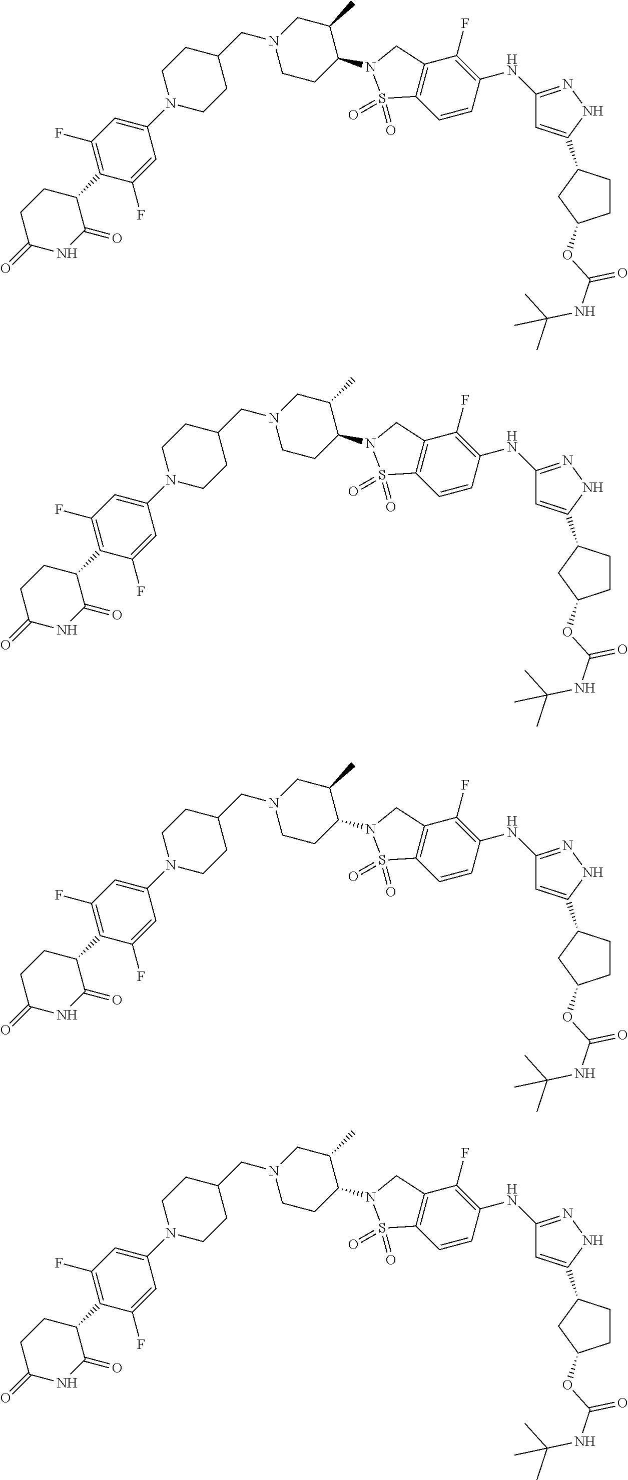

### Step 1: (R)-1-(4-(2,6-dioxopiperidin-3-yl)-3,5-difluorophenyl)piperidine-4-carbaldehyde

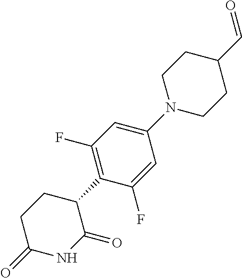 [SMILES: O=CC1CCN(c2cc(F)c([C@H]3CCC(=O)NC3=O)c(F)c2)CC1]

To a solution of (R)-3-(2,6-difluoro-4-(4-(hydroxymethyl)piperidin-1-yl)phenyl)piperidine-2,6-dione (2 g, 5.92 mmol, CN118359589A) in dichloromethane (30 mL) was added Dess-Martin periodinane (0.5 mL). The reaction mixture was stirred for 2 hours at room temperature. The resulting mixture was concentrated under reduce pressure. The residue was purified by silica gel column chromatography, eluting with PE/EA (1:3) to afford the product (1.8 g, 90.9% yield). LC-MS (ESI): m/z [M+H]⁺=337.2.

### Step 2: tert-butyl 4-(5-bromo-4-fluoro-1,1-dioxidobenzo[d]isothiazol-2(3H)-yl)-3-methylpiperidine-1-carboxylate

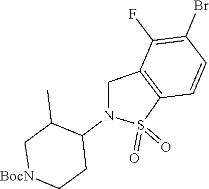 [SMILES: CC1CN(C(=O)OC(C)(C)C)CCC1N1Cc2c(ccc(Br)c2F)S1(=O)=O]

To a solution of 5-bromo-4-fluoro-2,3-dihydrobenzo[d]isothiazole 1,1-dioxide (2 g, 7.55 mmol) and tert-butyl 4-hydroxy-3-methylpiperidine-1-carboxylate (3.24 g, 15.1 mmol) in toluene (30 mL) was added 2-(tributylphosphoranylidene)acetonitrile (5.45 g, 22.65 mmol), The reaction mixture was stirred under nitrogen atmosphere at 100° C. for 16 h. The resulting mixture was concentrated under reduce pressure. The residue was purified by silica gel column chromatography, eluting with PE/EA (2:1) to afford the product (1.5 g, 42.8% yield). LC-MS (ESI): m/z [M+H]⁺=463.2.

### Step 3: tert-butyl 4-(5-((5-((1R,3S)-3-((tert-butylcarbamoyl)oxy)cyclopentyl)-1H-pyrazol-3-yl)amino)-4-fluoro-1,1-dioxidobenzo[d]isothiazol-2(3H)-yl)-3-methylpiperidine-1-carboxylate

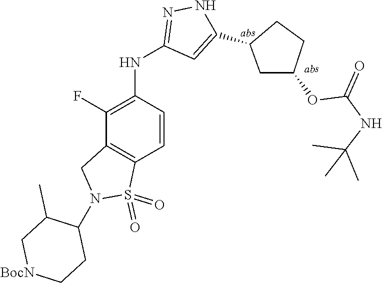 [SMILES: CC1CN(C(=O)OC(C)(C)C)CCC1N1Cc2c(ccc(Nc3cc([C@@H]4CC[C@H](OC(=O)NC(C)(C)C)C4)[nH]n3)c2F)S1(=O)=O]

To a mixture of tert-butyl 4-(5-bromo-4-fluoro-1,1-dioxidobenzo[d]isothiazol-2(3H)-yl)-3-methylpiperidine-1-carboxylate (300 mg, 0.71 mmol) and (1S,3R)-3-(3-amino-1H-pyrazol-5-yl)cyclopentyl tert-butylcarbamate (171 mg, 0.71 mmol) in t-BuOH (10 mL) was added BrettPhos Pd G3 (65 mg, 0.07 mmol) and K₂CO₃ (300 mg, 2.14 mmol). The reaction mixture was stirred under nitrogen atmosphere at 90° C. for 16 h. The mixture was filtered, and the filtrate was concentrated under reduced pressure. The residue was purified by silica gel column chromatography, eluting with DCM/MeOH (10:1) to afford the product (355 mg, 82% yield). LC-MS (ESI): m/z [M+H]⁺=649.3.

### Step 4: (1S,3R)-3-(3-((4-fluoro-2-(3-methylpiperidin-4-yl)-1,1-dioxido-2,3-dihydrobenzo[d]isothiazol-5-yl)amino)-1H-pyrazol-5-yl)cyclopentyl tert-butylcarbamate

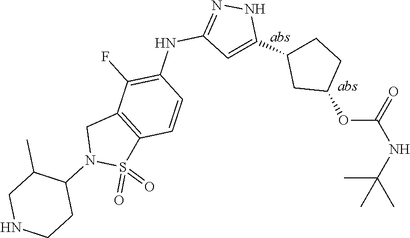 [SMILES: CC1CNCCC1N1Cc2c(ccc(Nc3cc([C@@H]4CC[C@H](OC(=O)NC(C)(C)C)C4)[nH]n3)c2F)S1(=O)=O]

tert-butyl 4-(5-((5-((1R,3S)-3-((tert-butylcarbamoyl)oxy)cyclopentyl)-1H-pyrazol-3-yl)amino)-4-fluoro-1,1-dioxidobenzo[d]isothiazol-2(3H)-yl)-3-methylpiperidine-1-carboxylate (300 mg, 0.46 mmol) was dissolved in 4M HCl (5 mL), and the reaction mixture was stirred for 2 hours at room temperature. The resulting mixture was concentrated under reduce pressure. Water was added and the pH was adjusted to 7 with triethylamine. The mixture was extracted by EA, dried over Na₂SO₄ and concentrated under vacuum to afford the product (251 mg, 100% yield). LC-MS (ESI): m/z [M+H]⁺=549.2.

### Step 5: (1S,3R)-3-(3-((2-((3R,4S)-1-((1-(4-((R)-2,6-dioxopiperidin-3-yl)-3,5-difluorophenyl)piperidin-4-yl)methyl)-3-methylpiperidin-4-yl)-4-fluoro-1,1-dioxido-2,3-dihydrobenzo[d]isothiazol-5-yl)amino)-1H-pyrazol-5-yl)cyclopentyl tert-butylcarbamate and

### (1S,3R)-3-(3-((2-((3S,4S)-1-((1-(4-((R)-2,6-dioxopiperidin-3-yl)-3,5-difluorophenyl)piperidin-4-yl)methyl)-3-methylpiperidin-4-yl)-4-fluoro-1,1-dioxido-2,3-dihydrobenzo[d]isothiazol-5-yl)amino)-1H-pyrazol-5-yl)cyclopentyl tert-butylcarbamate and

### (1S,3R)-3-(3-((2-((3R,4R)-1-((1-(4-((R)-2,6-dioxopiperidin-3-yl)-3,5-difluorophenyl)piperidin-4-yl)methyl)-3-methylpiperidin-4-yl)-4-fluoro-1,1-dioxido-2,3-dihydrobenzo[d]isothiazol-5-yl)amino)-1H-pyrazol-5-yl)cyclopentyl tert-butylcarbamate

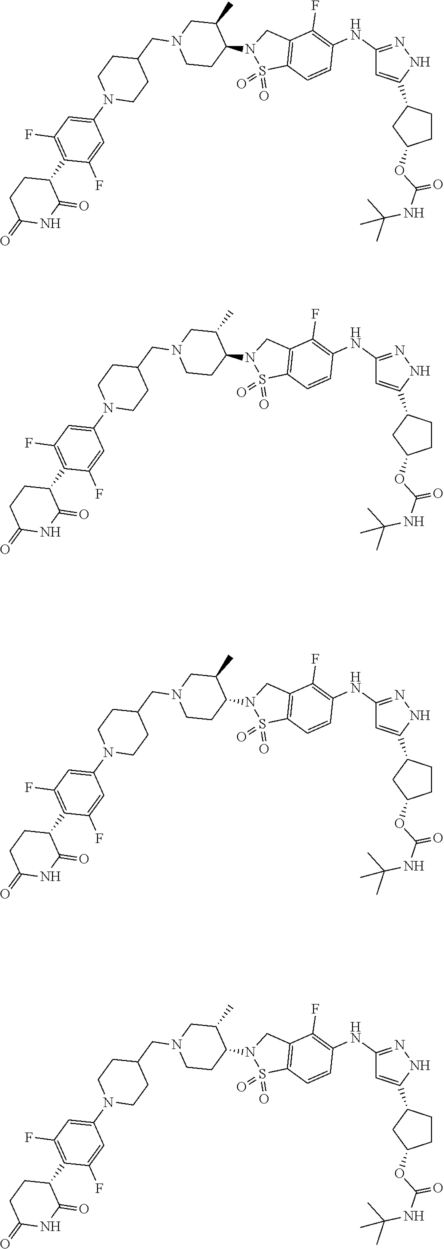

(1S,3R)-3-(3-((4-fluoro-2-(3-methylpiperidin-4-yl)-1,1-dioxido-2,3-dihydrobenzo[d]isothiazol-5-yl)amino)-1H-pyrazol-5-yl)cyclopentyl tert-butylcarbamate (100 mg, 0.17 mmol) and (R)-1-(4-(2,6-dioxopiperidin-3-yl)-3,5-difluorophenyl)piperidine-4-carbaldehyde (67 mg, 0.21 mmol) were dissolved in N,N-dimethylacetamide (3 mL) and stirred for 30 minutes. Sodium triacetoxyborohydride (60 mg, 0.28 mmol) was added, and the reaction mixture was stirred for 3 hours at room temperature. The resulting mixture was concentrated, and the residue was purified by column chromatography on silica gel, eluting with DCM/MeOH (10:1) to afford the product (80 mg, 54.8%) in a racemic form, which was further separated by Chiral Prep-HPLC to give:

Enantiomer 1 (Example 15a, 100% ee); Retention time: 10.54 min. ¹H NMR (500 MHz, DMSO) δ 11.98 (s, 1H), 10.86 (s, 1H), 8.77 (s, 1H), 8.31 (s, 1H), 7.52 (d, J=8.6 Hz, 1H), 6.76 (s, 1H), 6.61 (d, J=12.9 Hz, 2H), 5.83 (s, 1H), 5.06-4.96 (m, 1H), 4.55 (d, J=14.1 Hz, 1H), 4.44 (d, J=14.0 Hz, 1H), 4.04 (dd, J=12.7, 5.1 Hz, 1H), 3.75 (d, J=11.9 Hz, 2H), 3.29 (s, 3H), 3.12-3.02 (m, 1H), 2.96-2.86 (m, 2H), 2.86-2.68 (m, 3H), 2.47-2.41 (m, 1H), 2.22-1.86 (m, 10H), 1.80-1.65 (m, 7H), 1.63-1.53 (m, 1H), 1.21 (s, 9H), 1.17-1.05 (m, 2H). LC-MS (ESI): m/z [M+H]⁺=869.4.

Enantiomer 2 (Example 15b, 100% ee); Retention time: 13.75 min. ¹H NMR (500 MHz, DMSO) δ 11.98 (s, 1H), 10.86 (s, 1H), 8.77 (s, 1H), 8.31 (s, 1H), 7.52 (d, J=8.6 Hz, 1H), 6.76 (s, 1H), 6.61 (d, J=12.9 Hz, 2H), 5.83 (s, 1H), 5.06-4.96 (m, 1H), 4.55 (d, J=14.1 Hz, 1H), 4.44 (d, J=14.0 Hz, 1H), 4.04 (dd, J=12.7, 5.1 Hz, 1H), 3.75 (d, J=11.9 Hz, 2H), 3.29 (s, 3H), 3.12-3.02 (m, 1H), 2.96-2.86 (m, 2H), 2.86-2.68 (m, 3H), 2.47-2.41 (m, 1H), 2.22-1.86 (m, 10H), 1.80-1.65 (m, 7H), 1.63-1.53 (m, 1H), 1.21 (s, 9H), 1.17-1.05 (m, 2H). LC-MS (ESI): m/z [M+H]⁺=869.4.

Enantiomer 3 (Example 15c, 100% ee); Retention time: 15.15 min. ¹H NMR (500 MHz, DMSO) δ 11.98 (s, 1H), 10.86 (s, 1H), 8.77 (s, 1H), 8.31 (s, 1H), 7.52 (d, J=8.6 Hz, 1H), 6.76 (s, 1H), 6.61 (d, J=12.9 Hz, 2H), 5.83 (s, 1H), 5.06-4.96 (m, 1H), 4.55 (d, J=14.1 Hz, 1H), 4.44 (d, J=14.0 Hz, 1H), 4.04 (dd, J=12.7, 5.1 Hz, 1H), 3.75 (d, J=11.9 Hz, 2H), 3.29 (s, 3H), 3.12-3.02 (m, 1H), 2.96-2.86 (m, 2H), 2.86-2.68 (m, 3H), 2.47-2.41 (m, 1H), 2.22-1.86 (m, 10H), 1.80-1.65 (m, 7H), 1.63-1.53 (m, 1H), 1.21 (s, 9H), 1.17-1.05 (m, 2H). LC-MS (ESI): m/z [M+H]⁺=869.4.

Enantiomer 3 (Example 15d, 100% ee); Retention time: 18.188 min. ¹H NMR (500 MHz, DMSO) δ 11.98 (s, 1H), 10.86 (s, 1H), 8.77 (s, 1H), 8.31 (s, 1H), 7.52 (d, J=8.6 Hz, 1H), 6.76 (s, 1H), 6.61 (d, J=12.9 Hz, 2H), 5.83 (s, 1H), 5.06-4.96 (m, 1H), 4.55 (d, J=14.1 Hz, 1H), 4.44 (d, J=14.0 Hz, 1H), 4.04 (dd, J=12.7, 5.1 Hz, 1H), 3.75 (d, J=11.9 Hz, 2H), 3.29 (s, 3H), 3.12-3.02 (m, 1H), 2.96-2.86 (m, 2H), 2.86-2.68 (m, 3H), 2.47-2.41 (m, 1H), 2.22-1.86 (m, 10H), 1.80-1.65 (m, 7H), 1.63-1.53 (m, 1H), 1.21 (s, 9H), 1.17-1.05 (m, 2H). LC-MS (ESI): m/z [M+H]⁺=869.4.

Chiral analytical method: Column: ICHIRALPAK IE 4.6*150 mm 5 μm; Mobile phase: A for MtBE (0.1%2M NH3 MeOH) (%) and B for MeOH:DCM=50:50(%); Gradient: Mobile Phase A: Mobile Phase B=50:50 (v/v); HPLC Equipment: HPLC-Agilent, Back pressure: 100 bar; Column temperature: 35° C.

Chiral Prep-HPLC Condition: CHIRALPAK IE 250*20.0 mml, 5 μm; Mobile phase: A for MtBE (0.1%2M NH3 MeOH) (%) and B for MeOH:DCM=50:50; Gradient: Mobile Phase A: Mobile Phase B=50:50 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 16: (1S,3R)-3-(3-((2-((S)-1-((1-(4-((R)-2,6-dioxopiperidin-3-yl)-3,5-difluorophenyl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl tert-butylcarbamate

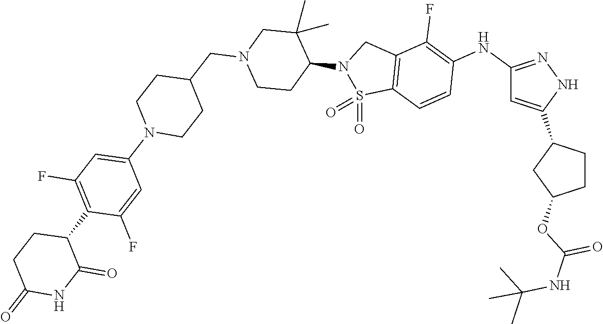 [SMILES: CC(C)(C)NC(=O)O[C@H]1CC[C@@H](c2cc(Nc3ccc4c(c3F)CN([C@H]3CCN(CC5CCN(c6cc(F)c([C@H]7CCC(=O)NC7=O)c(F)c6)CC5)CC3(C)C)S4(=O)=O)n[nH]2)C1]

### Step 1: tert-butyl 4-((4-bromo-3-fluoro-2-(methoxycarbonyl)phenyl) sulfonamido)-3,3-dimethylpiperidine-1-carboxylate

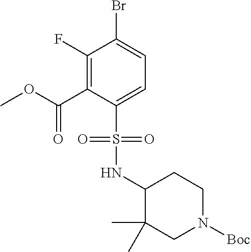 [SMILES: COC(=O)c1c(S(=O)(=O)NC2CCN(C(=O)OC(C)(C)C)CC2(C)C)ccc(Br)c1F]

To a solution of methyl 3-bromo-6-(chlorosulfonyl)-2-fluorobenzoate (220.0 g, 666.9 mmol, 1.0 eq) in ACN (3.2 L) and H₂O (640 mL) was added tert-butyl 4-amino-3,3-dimethylpiperidine-1-carboxylate (272.0 g, 1.0 mol, 1.5 eq) and Na₂CO₃ (204.6 g, 1.93 mol, 2.9 eq). The mixture was stirred for 3 h at 25° C. The reaction was quenched with H₂O (6.5 L) and extracted with EA (2.0 L×2). The combined organic phased were washed with brine (1.0 L), dried over Na₂SO₄, filtered and concentrated under reduced pressure. 2 batches (28.3 g, 320.0 g) were combined and purified by silica gel column (EtOAc/PE=5:1) to give the product (340.0 g, 98.5% purity, 91.0% assay, 81.4% yield) as a white solid. LCMS: 422.9/424.9 ([M−100]⁺).

### Step 2: tert-butyl 4-((4-bromo-3-fluoro-2-(hydroxymethyl)phenyl)sulfonamido)-3,3-dimethylpiperidine-1-carboxylate

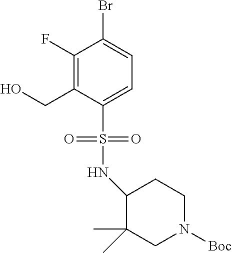 [SMILES: CC(C)(C)OC(=O)N1CCC(NS(=O)(=O)c2ccc(Br)c(F)c2CO)C(C)(C)C1]

To a solution of tert-butyl 4-((4-bromo-3-fluoro-2-(methoxycarbonyl)phenyl) sulfonamido)-3,3-dimethylpiperidine-1-carboxylate (330.0 g, 91.0% assay, 573.7 mmol, 1.0 eq) in THF (3.2 L) was added 2M LiBH₄ (410.0 mL, 820.0 mmol, 1.4 eq) dropwise at 25° C. The reaction was stirred at 55° C. for 2 h. The reaction mixture was cooled to 25° C. The reaction was quenched with 5% NH₄Cl (350 mL). The mixture was filtered. The filtrate was concentrated under reduced pressure. The residue was dissolved in DCM (3.0 L) and washed with brine (500 mL), dried over Na₂SO₄, concentrated under reduced pressure to give the product (291.5 g, 97.7% purity, 95.5% assay, 97.9% yield) as a yellow solid. LCMS: 395.0/397.0 ([M−100]⁺)

### Step 3: tert-butyl 4-((4-bromo-2-(bromomethyl)-3-fluorophenyl)sulfonamido)-3,3-dimethylpiperidine-1-carboxylate

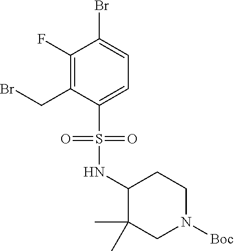 [SMILES: CC(C)(C)OC(=O)N1CCC(NS(=O)(=O)c2ccc(Br)c(F)c2CBr)C(C)(C)C1]

To a solution of tert-butyl 4-((4-bromo-3-fluoro-2-(hydroxymethyl)phenyl) sulfonamido)-3,3-dimethylpiperidine-1-carboxylate (240.0 g, 95.5% assay, 462.7 mmol, 1.0 eq) and CBr₄ (241.0 g, 726.7 mmol, 1.6 eq) in DCM (2.4 L) was added PPh₃ (254.1 g, 969.0 mol, 2.1 eq) in 6 portions at 0° C. The resulting mixture was stirred at 40° C. for 3 h. The reaction mixture was cooled to 25° C. The reaction mixture was washed with H₂O (720 mL), dried over Na₂SO₄, filtered and concentrated under reduced pressure. 2 batches (30.0 g, 240.0 g) were combined and purified by silica gel column (EtOAc/PE=5:1) to give the product (191.4 g, 98.2% purity, 93.3% assay, 61.5% yield) as a white solid. LCMS: 580.9 ([M+23]⁺)

### Step 4: tert-butyl 4-(5-bromo-4-fluoro-1,1-dioxidobenzo[d]isothiazol-2(3H)-yl)-3,3-dimethylpiperidine-1-carboxylate

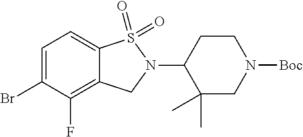 [SMILES: CC(C)(C)OC(=O)N1CCC(N2Cc3c(ccc(Br)c3F)S2(=O)=O)C(C)(C)C1]

To a solution of tert-butyl 4-((4-bromo-2-(bromomethyl)-3-fluorophenyl) sulfonamido)-3,3-dimethylpiperidine-1-carboxylate (187.0 g, 93.3% assay, 312.5 mmol, 1.0 eq) in ACN (2.2 L) and H₂O (380.0 mL) was added Na₂CO₃ (230.8 g, 2.2 mol, 7.0 eq). The resulting mixture was stirred at 80° C. for 16 h. The reaction mixture was cooled to 25° C. The reaction mixture was washed with H₂O (1.8 L). The mixture was extracted with EA (1.5 L×2). Phase separated. The combined organic phased were washed with brine (1.0 L), dried over Na₂SO₄, filtered and concentrated under reduced pressure. The residue was triturated with EtOAc/PE=2/1 (850 mL) at 25° C. for 1 h. The mixture was filtered, and the filter cake collected and dried (in oven at 50° C. for 12 h) to give the product (106.2 g, 99.3% purity, 98.0% assay, 69.8% yield) as a white solid. LCMS: 421.0/423.0 ([M−56+H]⁺).

### Step 5: tert-butyl 4-(5-((5-((1R,3S)-3-((tert-butylcarbamoyl)oxy)cyclopentyl)-1H-pyrazol-3-yl)amino)-4-fluoro-1,1-dioxidobenzo[d]isothiazol-2(3H)-yl)-3,3-dimethylpiperidine-1-carboxylate

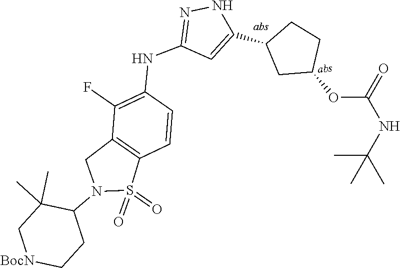 [SMILES: CC(C)(C)NC(=O)O[C@H]1CC[C@@H](c2cc(Nc3ccc4c(c3F)CN(C3CCN(C(=O)OC(C)(C)C)CC3(C)C)S4(=O)=O)n[nH]2)C1]

To a mixture of tert-butyl 4-(5-bromo-4-fluoro-1,1-dioxidobenzo[d]isothiazol-2(3H)-yl)-3,3-dimethylpiperidine-1-carboxylate (300 mg, 0.71 mmol) and (1S,3R)-3-(3-amino-1H-pyrazol-5-yl)cyclopentyl tert-butylcarbamate (170 mg, 0.71 mmol) in t-BuOH (10 mL) was added BrettPhos Pd G3 (65 mg, 0.07 mmol) and K₂CO₃ (300 mg, 2.14 mmol). The reaction mixture was stirred under nitrogen atmosphere at 100° C. for 16 h. The mixture was filtered, and the filtrate was concentrated under reduced pressure. The residue was purified by silica gel column chromatography, eluting with DCM/MeOH (10:1) to afford the product (355 mg, 82% yield). LC-MS (ESI): m/z [M+H]⁺=663.4.

### Step 6: (1S,3R)-3-(3-((2-(3,3-dimethylpiperidin-4-yl)-4-fluoro-1,1-dioxido-2,3-dihydrobenzo[d]isothiazol-5-yl)amino)-1H-pyrazol-5-yl)cyclopentyl tert-butylcarbamate

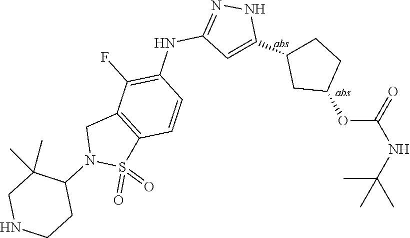 [SMILES: CC(C)(C)NC(=O)O[C@H]1CC[C@@H](c2cc(Nc3ccc4c(c3F)CN(C3CCNCC3(C)C)S4(=O)=O)n[nH]2)C1]

tert-butyl 4-(5-((5-((1R,3S)-3-((tert-butylcarbamoyl)oxy)cyclopentyl)-1H-pyrazol-3-yl)amino)-4-fluoro-1,1-dioxidobenzo[d]isothiazol-2(3H)-yl)-3,3-dimethylpiperidine-1-carboxylate (300 mg, 0.46 mmol) was dissolved in 4M HCl (5 mL), and the reaction mixture was stirred for 2 hours at room temperature. The resulting mixture was concentrated under reduce pressure. Water was added and the pH was adjusted to 7 with triethylamine. The mixture was extracted by EA, dried over Na₂SO₄ and concentrated under vacuum to afford the product (252 mg, 100% yield). LC-MS (ESI): m/z [M+H]⁺=563.3.

### Step 7: (1S,3R)-3-(3-((2-((S)-1-((1-(4-((R)-2,6-dioxopiperidin-3-yl)-3,5-difluorophenyl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl tert-butylcarbamate

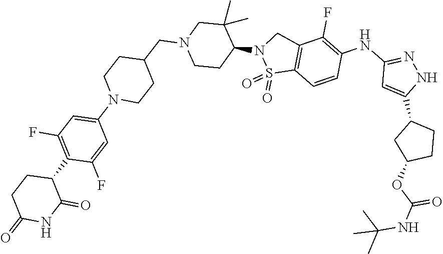 [SMILES: CC(C)(C)NC(=O)O[C@H]1CC[C@@H](c2cc(Nc3ccc4c(c3F)CN([C@H]3CCN(CC5CCN(c6cc(F)c([C@H]7CCC(=O)NC7=O)c(F)c6)CC5)CC3(C)C)S4(=O)=O)n[nH]2)C1]

(1S,3R)-3-(3-((2-(3,3-dimethylpiperidin-4-yl)-4-fluoro-1,1-dioxido-2,3-dihydrobenzo[d]isothiazol-5-yl)amino)-1H-pyrazol-5-yl)cyclopentyl tert-butylcarbamate (50 mg, 0.09 mmol) and (R)-1-(4-(2,6-dioxopiperidin-3-yl)-3,5-difluorophenyl)piperidine-4-carbaldehyde (36 mg, 0.11 mmol) were dissolved in N,N-dimethylacetamide (3 mL), stirred for 30 minutes, sodium triacetoxyborohydride (38 mg, 0.18 mmol) was added, the reaction mixture was stirred for 3 hours at room temperature. The resulting mixture was concentrated, the residue was purified by column chromatography on silica gel, eluting with DCM/MeOH (10:1) to afford the product (40 mg, 51.3%) in a racemic form, which was further separated by Chiral Prep-HPLC to give:

Enantiomer 1 (Example 16, 100% ee); Retention time: 4.576 min. ¹H NMR (500 MHz, DMSO) δ 11.98 (s, 1H), 10.86 (s, 1H), 8.77 (s, 1H), 8.31 (s, 1H), 7.54 (d, J=8.6 Hz, 1H), 6.76 (s, 1H), 6.61 (d, J=12.8 Hz, 2H), 5.83 (s, 1H), 5.06-4.96 (m, 1H), 4.56 (s, 2H), 4.04 (dd, J=12.5, 4.8 Hz, 1H), 3.76 (d, J=9.3 Hz, 2H), 3.29 (s, 1H), 3.22 (dd, J=12.1, 4.0 Hz, 1H), 3.10-3.02 (m, 1H), 2.97-2.92 (m, 1H), 2.83-2.70 (m, 3H), 2.57-2.53 (m, 2H), 2.47-2.43 (m, 1H), 2.23-1.86 (m, 9H), 1.84-1.65 (m, 6H), 1.64-1.55 (m, 1H), 1.50 (d, J=9.6 Hz, 1H), 1.21 (s, 9H), 1.12 (s, 3H), 0.93 (s, 3H). LC-MS (ESI): m/z [M+H]⁺=883.4.

Chiral analytical method: Column: ICHIRALPAK SA 4.6*150 mm 5 μm; Mobile phase: A for MtBE (0.1%2M NH3 MeOH) (%) and B for MeOH:DCM=50:50(%); Gradient: Mobile Phase A: Mobile Phase B=20:80 (v/v); HPLC Equipment: HPLC-Agilent, Back pressure: 100 bar; Column temperature: 35° C.

Chiral Prep-HPLC Condition: CHIRALPAK SA 250*20.0 mml, 5 μm; Mobile phase: A for MtBE (0.1%2M NH3 MeOH) (%) and B for MeOH:DCM=50:50; Gradient: Mobile Phase A: Mobile Phase B=20:80 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 17a and 17b: (1S,3R)-3-(3-((2-((S)-1-((1-(4-((R)-2,6-dioxopiperidin-3-yl)-3,5-difluorophe-nyl)piperidin-4-yl)methyl)-3,3-difluoropiperidin-4-yl)-4-fluoro-1,1-dioxido-2,3-dihydro-benzo[d]isothiazol-5-yl)amino)-1H-pyrazol-5-yl)cyclopentyl tert-butylcarbamate and

### (1S,3R)-3-(3-((2-((R)-1-((1-(4-((R)-2,6-dioxopiperidin-3-yl)-3,5-difluorophenyl)piperidin-4-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl tert-butylcarbamate

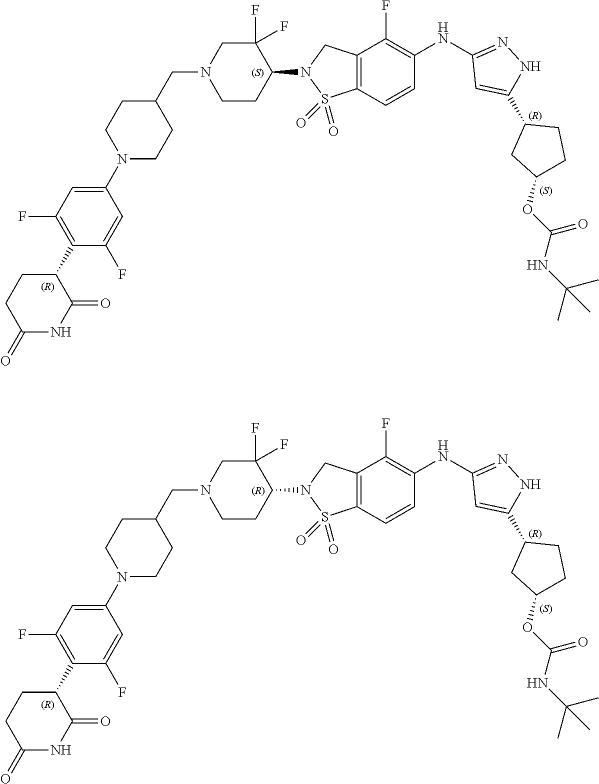

### Step 1: tert-butyl 4-((4-bromo-3-fluoro-2-(methoxycarbonyl)phenyl) sulfonamido)-3,3-difluoropiperidine-1-carboxylate

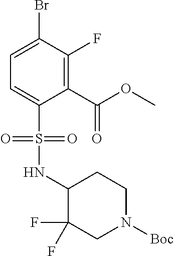 [SMILES: COC(=O)c1c(S(=O)(=O)NC2CCN(C(=O)OC(C)(C)C)CC2(F)F)ccc(Br)c1F]

To a suspension of methyl 3-bromo-6-(chlorosulfonyl)-2-fluorobenzoate (206 g, 625.3 mmol, 1.0 eq) in ACN (2.0 L) and H₂O (420 mL) was added tert-butyl 4-amino-3,3-difluoropiperidine-1-carboxylate (219.4 g, 80.8% assay, 750.3 mmol, 1.2 eq) and Na₂CO₃ (132.5 g, 1.25 mol, 2.0 eq). The mixture was stirred for 3 h at 25° C. The reaction was quenched with H₂O (4.1 L) and extracted with EtOAc (2.1 L×2). The combined organic phased were washed with brine (1.0 L), dried over Na₂SO₄, filtered and concentrated under reduced pressure. 2 batches (20.0 g, 300.0 g) were combined and purified by silica gel column chromatography (Petroleum ether/EtOAc=5:1) to give the product (196.5 g, 98.7% assay, 54.7% yield) as a white solid. LCMS: 432.9, 430.9 ([M−100+H]⁺).

### Step 2: tert-butyl 4-((4-bromo-3-fluoro-2-(hydroxymethyl)phenyl)sulfonamido)-3,3-difluoropiperidine-1-carboxylate

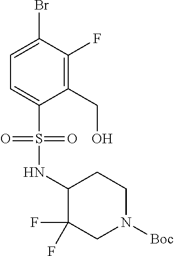 [SMILES: CC(C)(C)OC(=O)N1CCC(NS(=O)(=O)c2ccc(Br)c(F)c2CO)C(F)(F)C1]

To a solution of tert-butyl 4-((4-bromo-3-fluoro-2-(methoxycarbonyl)phenyl) sulfonamido)-3,3-difluoropiperidine-1-carboxylate (160.0 g, 98.7% assay, 297.2 mmol, 1.0 eq) in THF (1.6 L) was added LiBH₄ in THF (195.0 mL, 2M, 390.0 mmol, 1.3 eq) dropwise at 25° C. The reaction was stirred at 60° C. for 2 h. The reaction mixture was cooled to 25° C., quenched with 5% NH₄Cl aqueous (200 mL) and filtered. The filtrate was concentrated under reduced pressure. The residue was dissolved in DCM (1.0 L), washed with brine (500 mL), dried over Na₂SO₄ and concentrated under reduced pressure to give the product (161.2 g, 92.4% assay, 99.6% yield) as a white solid. LCMS: 402.9, 404.9 ([M−100+H]⁺).

### Step 3: tert-butyl 4-((4-bromo-2-(bromomethyl)-3-fluorophenyl)sulfonamido)-3,3-difluoropiperidine-1-carboxylate

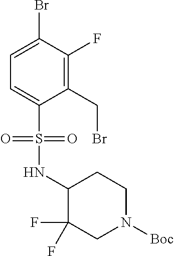 [SMILES: CC(C)(C)OC(=O)N1CCC(NS(=O)(=O)c2ccc(Br)c(F)c2CBr)C(F)(F)C1]

To a solution of tert-butyl 4-((4-bromo-3-fluoro-2-(hydroxymethyl)phenyl) sulfonamido)-3,3-difluoropiperidine-1-carboxylate (130.0 g, 92.4% assay, 238.7 mmol, 1.0 eq) and CBr₄ (118.7 g, 358.0 mmol, 1.5 eq) in DCM (1.2 L) was added PPh₃ (125.2 g, 477.3 mmol, 2.0 eq) in 12 portions at 0° C. The resulting mixture was stirred at 40° C. for 3 h. The reaction mixture was cooled to 25° C., washed with H₂O (1.2 L), dried over Na₂SO₄, filtered and concentrated under reduced pressure. The mixture was combined and purified by silica gel column chromatography (Petroleum ether/EtOAc=5:1) to give the product (66.9 g, 95.2% assay, 47.1% yield) as a white solid. LCMS: 588.8 ([M+Na]⁺).

### Step 4: tert-butyl 4-(5-bromo-4-fluoro-1,1-dioxidobenzo[d]isothiazol-2(3H)-yl)-3,3-difluoropiperidine-1-carboxylate

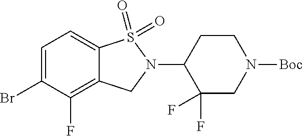 [SMILES: CC(C)(C)OC(=O)N1CCC(N2Cc3c(ccc(Br)c3F)S2(=O)=O)C(F)(F)C1]

To a solution of tert-butyl 4-((4-bromo-2-(bromomethyl)-3-fluorophenyl) sulfonamido)-3,3-difluoropiperidine-1-carboxylate (20.0 g, 95.2% assay, 33.6 mmol, 1.0 eq) in ACN (300 mL) and H₂O (40 mL) was added Na₂CO₃ (23.2 g, 218.6 mmol, 6.5 eq). The resulting mixture was stirred at 80° C. for 3 h. The reaction mixture was cooled to 25° C., washed with H₂O (200 mL) and extracted with EtOAc (200 mL×2). The combined organic phased were washed with brine (100 mL), dried over Na₂SO₄ and filtered. 5 batches (20.0 g×4, 10.0 g) were combined and concentrated under reduced pressure. The residue was triturated with EtOAc/Petroleum ether (850 mL, 2:1) at 25° C. for 1 h and filtered, and the filter cake collected and dried (in oven at 50° C. for 12 h) to give the product (60.0 g, 81.4% yield) as a white solid. LCMS: 428.9, 430.9 ([M−56+H]⁺).

### Step 5: tert-butyl 4-(5-((5-((1R,3S)-3-((tert-butylcarbamoyl)oxy)cyclopentyl)-1H-pyrazol-3-yl)amino)-4-fluoro-1,1-dioxidobenzo[d]isothiazol-2(3H)-yl)-3,3-difluorop-iperidine-1-carboxylate

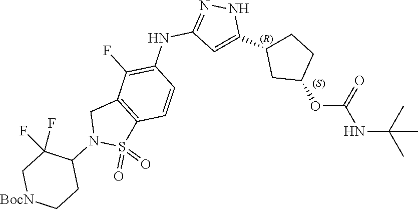

To a mixture of tert-butyl 4-(5-bromo-4-fluoro-1,1-dioxidobenzo[d]isothiazol-2(3H)-yl)-3,3-difluoropiperidine-1-carboxylate (90 mg, 0.18 mmol) and ((1S,3R)-3-(3-amino-1H-pyrazol-5-yl)cyclopentyl tert-butylcarbamate (50 mg, 0.18 mmol) in t-BuOH (5 mL) was added Brettphos Pd G3 (16.8 mg, 0.018 mmol) and K₂CO₃ (75 mg, 0.18 mmol). The reaction mixture was stirred under nitrogen atmosphere at 110° C. for 16 h. The mixture was concentrated under reduced pressure. The residue was purified by silica gel column chromatography, eluting with PE/EA (1:9) to afford the product (70 mg, 56% yield). LC-MS (ESI): m/z [M+H]⁺=671.3.

### Step 6: (1S,3R)-3-(3-((2-(3,3-difluoropiperidin-4-yl)-4-fluoro-1,1-dioxido-2,3-dihydrob-enzo[d]isothiazol-5-yl)amino)-1H-pyrazol-5-yl)cyclopentyl tert-butylcarbamate

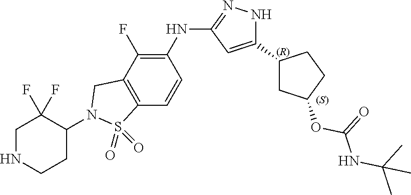

To a solution of tert-butyl 4-(5-((5-((1R,3S)-3-((tert-butylcarbamoyl)oxy)cyclopentyl)-1H-pyrazol-3-yl)amino)-4-fluoro-1,1-dioxidobenzo[d]isothiazol-2(3H)-yl)-3,3-difluorop-iperidine-1-carboxylate (70 mg, 0.104 mmol) in dichloromethane (4 mL) was added trifluoroacetic acid (2 mL). The reaction mixture was stirred for 1 hours at room temperature. The resulting mixture was concentrated, dried over in vacuum, to afford the product (59 mg, 100% yield). LC-MS (ESI): m/z [M+H]⁺=571.2.

### Step 7: (1S,3R)-3-(3-((2-((S)-1-((1-(4-((R)-2,6-dioxopiperidin-3-yl)-3,5-difluorophe-nyl)piperidin-4-yl)methyl)-3,3-difluoropiperidin-4-yl)-4-fluoro-1,1-dioxido-2,3-dihydro-benzo[d]isothiazol-5-yl)amino)-1H-pyrazol-5-yl)cyclopentyl tert-butylcarbamate and (1S,3R)-3-(3-((2-((R)-1-((1-(4-((R)-2,6-dioxopiperidin-3-yl)-3,5-difluorophenyl)piperidin-4-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl tert-butylcarbamate

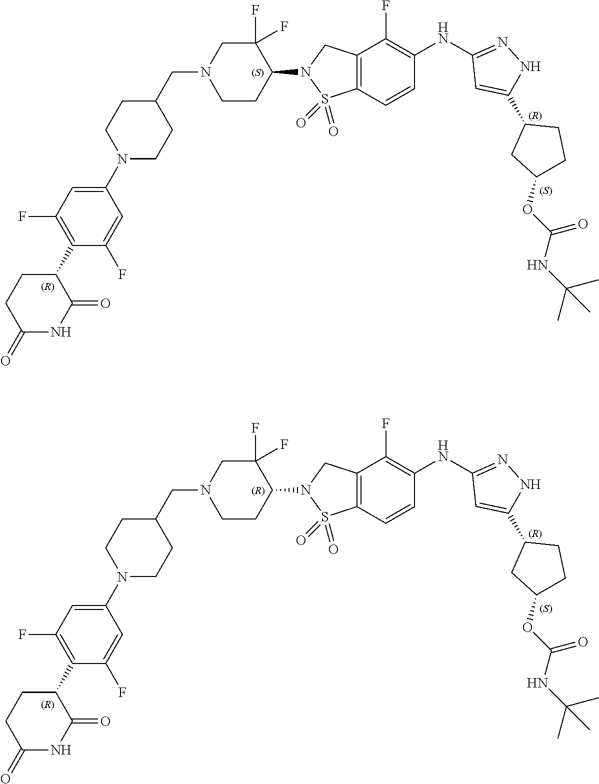

To a solution of (1S,3R)-3-(3-((2-(3,3-difluoropiperidin-4-yl)-4-fluoro-1,1-dioxido-2,3-dihydrob-enzo[d]isothiazol-5-yl)amino)-1H-pyrazol-5-yl)cyclopentyl tert-butyl carbamate (60 mg, 0.105 mmol), (1S,3R)-3-(3-((2-(3,3-difluoropiperidin-4-yl)-4-fluoro-1,1-dioxido-2,3-dihydrobenzo[d]isothiazol-5-yl)amino)-1H-pyrazol-5-yl)cyclopentyl tert-butylcarbamate (53 mg, 0.158 mmol) and Sodium acetate (53 mg, 0.650 mmol) in DMA (3 mL) was stirred for 30 min, then STAB (45 mg, 0.21 mmol) was added. the reaction mixture was stirred for 16 hours at room temperature. The resulting mixture was concentrated under reduce pressure, the residue was purified by column chromatography on silica gel, eluting with DCM/MeOH (10:1) to afford the product (35 mg, 37%). in a racemic form, which was further separated by Chiral Prep-HPLC to give:

Enantiomer 1 (example 17a, 100% ee); Retention time: 5.575 min. ¹H NMR (500 MHz, DMSO) δ 12.00 (s, 1H), 10.86 (s, 1H), 8.83 (s, 1H), 8.33 (s, 1H), 7.58 (d, J=8.7 Hz, 1H), 6.76 (s, 1H), 6.62 (d, J=12.9 Hz, 2H), 5.84 (s, 1H), 4.98 (s, 1H), 4.64 (dd, J=105.4 Hz, 13.9 Hz, 2H), 4.07-3.94 (m, 2H), 3.76 (d, J=10.9 Hz, 2H), 3.29 (s, 1H), 3.16 (t, J=10.0 Hz, 1H), 3.11-3.01 (m, 1H), 2.93 (d, J=9.8 Hz, 1H), 2.83-2.69 (m, 3H), 2.38-2.25 (m, 3H), 2.23-1.87 (m, 6H), 1.82-1.68 (m, 6H), 1.59 (s, 1H), 1.24-1.10 (m, 12H). LC-MS (ESI): m/z [M+H]⁺=891.54.

Enantiomer 2 (example 17b, 100% ee); Retention time: 9.92 min. ¹H NMR (500 MHz, DMSO) δ 12.00 (s, 1H), 10.86 (s, 1H), 8.83 (s, 1H), 8.33 (s, 1H), 7.58 (d, J=8.7 Hz, 1H), 6.76 (s, 1H), 6.62 (d, J=12.9 Hz, 2H), 5.84 (s, 1H), 4.98 (s, 1H), 4.64 (dd, J=105.4, 13.9 Hz, 2H), 4.07-3.94 (m, 2H), 3.76 (d, J=10.9 Hz, 2H), 3.29 (s, 1H), 3.16 (t, J=10.0 Hz, 1H), 3.11-3.01 (m, 1H), 2.93 (d, J=9.8 Hz, 1H), 2.83-2.69 (m, 3H), 2.38-2.25 (m, 3H), 2.23-1.87 (m, 6H), 1.82-1.68 (m, 6H), 1.59 (s, 1H), 1.24-1.10 (m, 12H). LC-MS (ESI): m/z [M+H]⁺=891.71.

Chiral analytical method: Column: CHIRALPAK IE 4.6*250 mm 5 μm; Mobile phase: A for MtBE (0.1%2M NH3 MeOH)(%) and B for MeOH:DCM=50:50 (v/v); Gradient: Mobile Phase A: Mobile Phase B=20:80 (v/v); HPLC Equipment: Agilent 1260 Infinity II (ARD_LC-808); Column temperature: 25° C.

Chiral Prep-HPLC Condition: CHIRALPAK IE 250*20.0 mml, 5 μm; Mobile phase: A for MtBE (0.1%2M NH3 MeOH)(%) and B for MeOH:DCM=50:50; Gradient: Mobile Phase A: Mobile Phase B=20:80 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 18: (1S,3R)-3-(3-((2-((cis-4-((4-(4-((R)-2,6-dioxopiperidin-3-yl)-3,5-difluorophenyl)piperazin-1-yl)methyl)cyclohexyl)-4-fluoro-1,1-dioxido-2,3-dihydrobenzo [d]isothiazol-5-yl)amino)-1H-pyrazol-5-yl)cyclopentyl tert-butylcarbamate

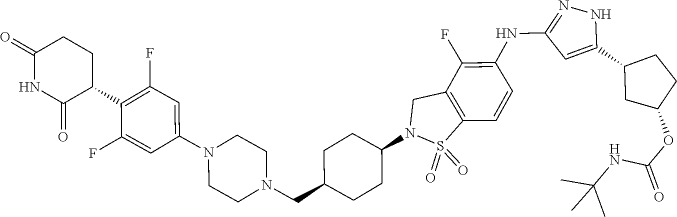 [SMILES: CC(C)(C)NC(=O)O[C@H]1CC[C@@H](c2cc(Nc3ccc4c(c3F)CN([C@H]3CC[C@@H](CN5CCN(c6cc(F)c([C@H]7CCC(=O)NC7=O)c(F)c6)CC5)CC3)S4(=O)=O)n[nH]2)C1]

### Step 1: trans-4-(((tert-butyldiphenylsilyl)oxy)methyl)cyclohexan-1-ol

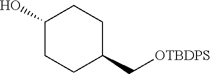

To a solution of trans-4-(hydroxymethyl)cyclohexan-1-ol (500 mg, 3.84 mmol) and imidazole (783 mg, 11.52 mmol) in dichloromethane (10 mL) was added TBDPSCl (1.06 g, 3.84 mmol), the reaction mixture was stirred for 16 hours at room temperature. The resulting mixture was concentrated, The residue was purified by silica gel column chromatography, eluting with PE/EA (4:1, R_{f}=0.3) to afford the product (326 mg, 23% yield). ¹H NMR (500 MHz, DMSO) δ=7.60 (m, 4H), 7.48-7.41 (m, 6H), 4.48 (d, J=4.4, 1H), 3.44 (d, J=6.2, 2H), 3.29 (m, 1H), 1.82 (m, 2H), 1.75-1.69 (m, 2H), 1.49-1.34 (m, 1H), 1.15-1.04 (m, 2H), 0.99 (s, 9H), 0.98-0.89 (m, 2H).

### Step 2: (1S,3R)-3-(3-((2-(cis-4-(((tert-butyldiphenylsilyl)oxy)methyl)cyclohexyl)-4-fluoro-1,1-dioxido-2,3-dihydrobenzo[d]isothiazol-5-yl)amino)-1H-pyrazol-5-yl)cyclopentyl tert-butylcarbamate

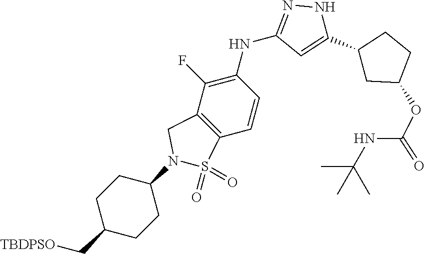

To a solution of trans-4-(((tert-butyldiphenylsilyl)oxy)methyl)cyclohexan-1-ol (200 mg, 0.543 mmol) and (1S,3R)-3-(3-((4-fluoro-1,1-dioxido-2,3-dihydrobenzo[d]isothiazol-5-yl)amino)-1H-pyrazol-5-yl)cyclopentyl tert-butylcarbamate (258 mg, 0.571 mmol) in toluene (10 mL) was added CMBP (393 mg, 1.63 mmol), the reaction mixture was stirred for 16 hours at 100° C. under nitrogen. The resulting mixture was cooled to room temperature, concentrated under reduce pressure, the residue was purified by silica gel column chromatography, eluting with PE/EA (1:1) to afford the product (250 mg, 57% yield). LC-MS (ESI): m/z [M+H]⁺=802.5.

### Step 3: (1S,3R)-3-(3-((4-fluoro-2-(cis-4-(hydroxymethyl)cyclohexyl)-1,1-dioxido-2,3-dihydrobenzo[d]isothiazol-5-yl)amino)-1H-pyrazol-5-yl)cyclopentyl tert-butylcarbamate

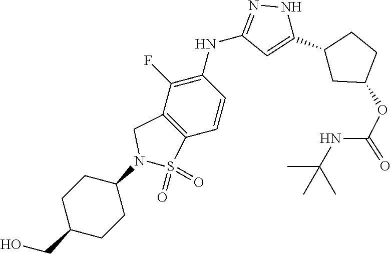 [SMILES: CC(C)(C)NC(=O)O[C@H]1CC[C@@H](c2cc(Nc3ccc4c(c3F)CN([C@H]3CC[C@@H](CO)CC3)S4(=O)=O)n[nH]2)C1]

To s solution of (1S,3R)-3-(3-((2-(cis-4-(((tert-butyldiphenylsilyl)oxy)methyl) cyclohexyl)-4-fluoro-1,1-dioxido-2,3-dihydrobenzo[d]isothiazol-5-yl)amino)-1H-pyrazol-5-yl)cyclopentyl tert-butylcarbamate (250 mg, 0.313 mmol) in THF (5 mL) was added TBAF·H₂O (245 mg, 0.936 mmol), the reaction mixture was stirred for 16 hours at room temperature. The resulting mixture was concentrated under reduce pressure, the residue was purified by silica gel column chromatography, eluting with PE/EA (1:1-EA) to afford the product (125 mg, 71% yield). LC-MS (ESI): m/z [M+H]⁺=564.2

### Step 4: (1S,3R)-3-(3-((4-fluoro-2-(cis-4-formylcyclohexyl)-1,1-dioxido-2,3-dihydrobenzo[d]isothiazol-5-yl)amino)-1H-pyrazol-5-yl)cyclopentyl tert-butylcarbamate

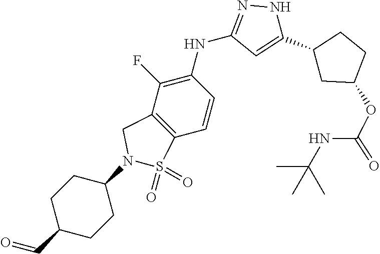 [SMILES: CC(C)(C)NC(=O)O[C@H]1CC[C@@H](c2cc(Nc3ccc4c(c3F)CN([C@H]3CC[C@@H](C=O)CC3)S4(=O)=O)n[nH]2)C1]

To a solution of (1S,3R)-3-(3-((4-fluoro-2-(cis-4-(hydroxymethyl)cyclohexyl)-1,1-dioxido-2,3-dihydrobenzo[d]isothiazol-5-yl)amino)-1H-pyrazol-5-yl)cyclopentyl tert-butylcarbamate (125 mg, 0.222 mmol) in dichloromethane (5 mL) was added DMP (282 mg, 0.666 mmol), the resulting mixture was stirred for 2 hours at room temperature. The resulting mixture was filtered, the filtrate was concentrated under reduce pressure, the residue was purified by silica gel column chromatography, eluting with PE/EA (1:4) to afford the product (50 mg, 40% yield). LC-MS (ESI): m/z [M+H]⁺=562.2

### Step 5: (1S,3R)-3-(3-((2-((cis-4-((4-(4-((R)-2,6-dioxopiperidin-3-yl)-3,5-difluorophenyl)piperazin-1-yl)methyl)cyclohexyl)-4-fluoro-1,1-dioxido-2,3-dihydrobenzo [d]isothiazol-5-yl)amino)-1H-pyrazol-5-yl)cyclopentyl tert-butylcarbamate

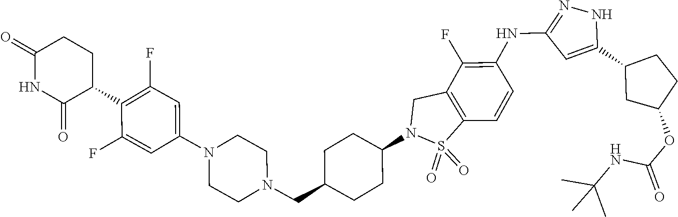 [SMILES: CC(C)(C)NC(=O)O[C@H]1CC[C@@H](c2cc(Nc3ccc4c(c3F)CN([C@H]3CC[C@@H](CN5CCN(c6cc(F)c([C@H]7CCC(=O)NC7=O)c(F)c6)CC5)CC3)S4(=O)=O)n[nH]2)C1]

The titled compound was synthesized in the procedures similar to Example 2. LC-MS (ESI): m/z [M+H]⁺=855.68; ¹H NMR (500 MHz, DMSO) δ=11.98 (s, 1H), 10.87 (s, 1H), 8.77 (s, 1H), 8.32 (d, J=10.4, 1H), 7.50 (d, J=8.6, 1H), 6.76 (s, 1H), 6.63 (d, J=12.6, 2H), 5.83 (s, 1H), 4.98 (s, 1H), 4.48 (d, J=16.3, 2H), 4.05 (dd, J=12.4, 5.0, 1H), 3.48 (m, 1H), 3.19 (m, 4H), 3.08-3.02 (m, 1H), 2.79 (m, 1H), 2.47 (m, 5H), 2.26 (m, 2H), 2.10-1.89 (m, 6H), 1.68 (m, 11H), 1.21 (s, 9H).

### Example 19: (1S,3R)-3-(3-((2-(1-((1-(4-(2,4-dioxotetrahydropyrimidin-1(2H)-yl)phenyl) piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

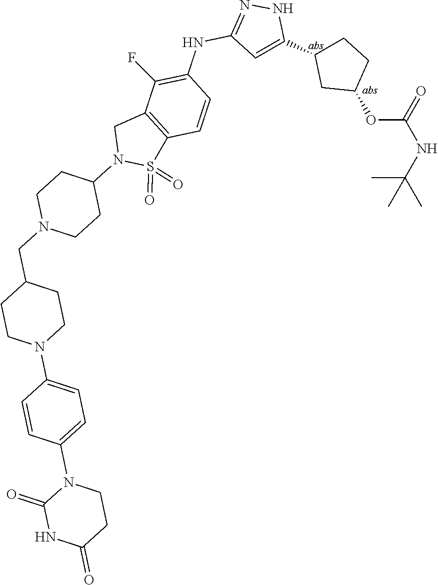 [SMILES: CC(C)(C)NC(=O)O[C@H]1CC[C@@H](c2cc(Nc3ccc4c(c3F)CN(C3CCN(CC5CCN(c6ccc(N7CCC(=O)NC7=O)cc6)CC5)CC3)S4(=O)=O)n[nH]2)C1]

The titled compound was synthesized in the procedures similar to Example 1. ¹H NMR (500 MHz, DMSO-d₆) δ 11.98 (s, 1H), 10.25 (s, 1H), 8.77 (s, 1H), 8.28 (s, 1H), 7.50 (d, J=10 Hz, 1H), 7.14-7.11 (m, 2H), 6.95-6.80 (m, 2H), 6.79-6.73 (m, 1H), 5.83 (s, 1H), 5.01-4.94 (m, 1H), 4.51 (s, 2H), 3.72-3.64 (m, 4H), 3.47-3.39 (m, 1H), 3.10-3.01 (m, 1H), 2.93-2.86 (m, 2H), 2.70-2.61 (m, 4H), 2.46-2.43 (m, 1H), 2.20-2.15 (m, 2H), 2.07-1.98 (m, 3H), 1.94-1.75 (m, 7H), 1.74-1.55 (m, 4H), 1.26-1.22 (m, 2H), 1.21 (s, 9H). LC-MS (ESI): m/z [M+H]⁺=820.

### Example 20: (1S,3R)-3-(3-((2-(1-((1-(4-(2,4-dioxotetrahydropyrimidin-1(2H)-yl)-3-methylphenyl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

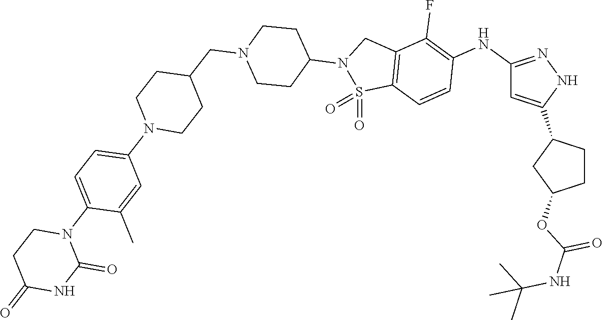 [SMILES: Cc1cc(N2CCC(CN3CCC(N4Cc5c(ccc(Nc6cc([C@@H]7CC[C@H](OC(=O)NC(C)(C)C)C7)[nH]n6)c5F)S4(=O)=O)CC3)CC2)ccc1N1CCC(=O)NC1=O]

### Step 1: (1-(3-methyl-4-nitrophenyl)piperidin-4-yl)methanol

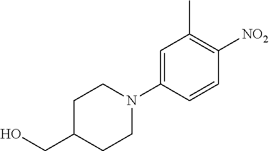 [SMILES: Cc1cc(N2CCC(CO)CC2)ccc1[N+](=O)[O-]]

To a solution of 4-fluoro-2-methyl-1-nitrobenzene (5.0 g, 32.23 mmol) and piperidin-4-ylmethanol (4.45 g, 38.68 mmol) in DMF (50 mL) was added K₂CO₃ (8.9 g, 64.46 mmol), the reaction mixture was stirred for 2 hours at 90° C. The resulting mixture was cooled to room temperature, diluted with water, extracted with EtOAc for 3 times, the combined organic phase was washed with brine, dried over Na₂SO₄, filtered, the filtrated was concentrated under reduce pressure, the residue was purified by silica gel column chromatography, eluting with PE/EA (1:3) to afford the product (7.5 g, 93% yield). LC-MS (ESI): m/z [M+H]⁺=251.2

### Step 2: (1-(4-amino-3-methylphenyl)piperidin-4-yl)methanol

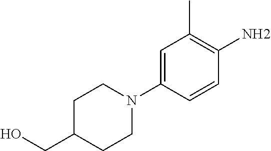 [SMILES: Cc1cc(N2CCC(CO)CC2)ccc1N]

To a solution of (1-(3-methyl-4-nitrophenyl)piperidin-4-yl)methanol (2.5 g, 10 mmol) in THF (50 mL) was added Pd/C (10% on carbon, 500 mg). The suspension was degassed and purged with hydrogen 3 times. The resulting mixture was stirred at room temperature under a hydrogen balloon for 2 h. The mixture was filtered, and the filter cake was washed with EA (20 mL×3). The filtrate was concentrated under reduced pressure to afford the product (2.2 g, 100% yield). LC-MS (ESI): m/z [M+H]⁺=221.2.

### Step 3: (1-(4-(2,4-dioxotetrahydropyrimidin-1(2H)-yl)-3-methylphenyl)piperidin-4-yl)methyl acetate

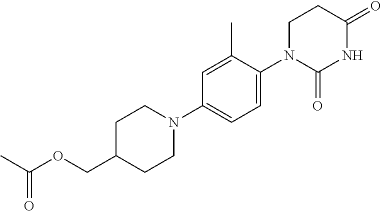 [SMILES: CC(=O)OCC1CCN(c2ccc(N3CCC(=O)NC3=O)c(C)c2)CC1]

(1-(4-amino-3-methylphenyl)piperidin-4-yl)methanol (2.2 g, 10 mmol) and acrylic acid (2.16 g, 30 mmol) were dissolved in toluene, heated to 90° C., stirred for 3 hours. The mixture was cool to room temperature, urea (4.8 g, 80 mmol) and AcOH (12 mL) were then added, the reaction mixture was stirred for 16 hours at 100° C. The resulting mixture was cooled to room temperature, concentrated under reduce pressure, saturated Na₂CO₃ aqueous was added, extracted with EtOAc for 3 times, the combined organic phase was washed with brine, dried over Na₂SO₄, filtered, the filtrated was concentrated under reduce pressure, the residue was purified by silica gel column chromatography, eluting with DCM/MeOH (10:1) to afford the product (1.55 g, 43% yield). LC-MS (ESI): m/z [M+H]⁺=360.2

### Step 4: 1-(4-(4-(hydroxymethyl)piperidin-1-yl)-2-methylphenyl)dihydropyrimidine-2,4(1H,3H)-dione

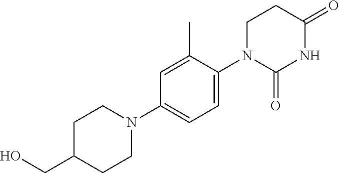 [SMILES: Cc1cc(N2CCC(CO)CC2)ccc1N1CCC(=O)NC1=O]

(1-(4-(2,4-dioxotetrahydropyrimidin-1(2H)-yl)-3-methylphenyl)piperidin-4-yl)methyl acetate (1.55 g, 4.32 mmol) was dissolved in HCl aqueous (4N, 20 mL), the reaction mixture was stirred for 1 hour at 100° C. The resulting mixture was cooled to room temperature, adjusted PH to 7 with addition of saturate NaHCO₃ aqueous, a precipitated was formed, filtered, washed with water, dried over in vacuum, to afford the product (1.15 g, 84%).

### Step 5: 1-(4-(2,4-dioxotetrahydropyrimidin-1(2H)-yl)-3-methylphenyl)piperidine-4-carbaldehyde

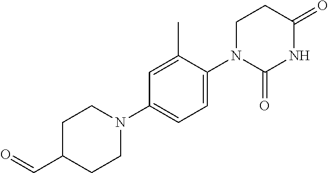 [SMILES: Cc1cc(N2CCC(C=O)CC2)ccc1N1CCC(=O)NC1=O]

To a solution of 1-(4-(4-(hydroxymethyl)piperidin-1-yl)-2-methylphenyl) dihydropyrimidine-2,4(1H,3H)-dione (100 mg, 0.315 mmol) in DMSO (3 mL) was added IBX (177 mg, 0.63 mmol), the reaction mixture was stirred for 16 hours at room temperature. The resulting mixture was dilute with water, extracted with EtOAc for 3 times, the combined organic phase was washed with brine, dried over Na₂SO₄, filtered, the filtrated was concentrated under reduce pressure, dried over in vacuum, to afford the product (80 mg, 80% yield). LC-MS (ESI): m/z [M+H]⁺=316.4

### Step 5: (1S,3R)-3-(3-((2-(1-((1-(4-(2,4-dioxotetrahydropyrimidin-1(2H)-yl)-3-methylphenyl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

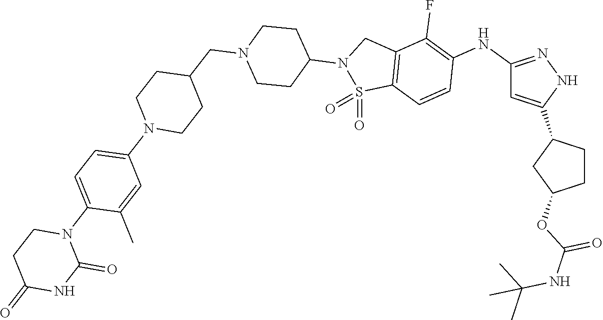 [SMILES: Cc1cc(N2CCC(CN3CCC(N4Cc5c(ccc(Nc6cc([C@@H]7CC[C@H](OC(=O)NC(C)(C)C)C7)[nH]n6)c5F)S4(=O)=O)CC3)CC2)ccc1N1CCC(=O)NC1=O]

The titled compound was synthesized in the procedures similar to Example 19. LC-MS (ESI): m/z [M+H]⁺=834.68; ¹H NMR (500 MHz, DMSO) δ=11.99 (s, 1H), 10.23 (s, 1H), 8.77 (s, 1H), 8.29 (s, 1H), 7.51 (d, J=8.6, 1H), 7.04 (d, J=8.6, 1H), 6.81 (d, J=2.5, 1H), 6.77 (dd, J=8.7, 2.6, 2H), 5.83 (s, 1H), 4.97 (s, 1H), 4.52 (s, 2H), 3.67 (m, 3H), 3.50-3.42 (m, 2H), 3.10-3.02 (m, 1H), 2.91 (d, J=11.0, 2H), 2.73-2.63 (m, 4H), 2.45 (m, 1H), 2.18 (d, J=7.0, 2H), 2.11 (m, 3H), 2.08-2.00 (m, 3H), 1.89 (m, 5H), 1.78 (m, 2H), 1.75-1.63 (m, 3H), 1.58 (m, 1H), 1.25-1.15 (m, 11H).

### Example 21: (1S,3R)-3-(3-((2-(1-((1-(4-(2,4-dioxotetrahydropyrimidin-1(2H)-yl)-3-fluorophenyl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

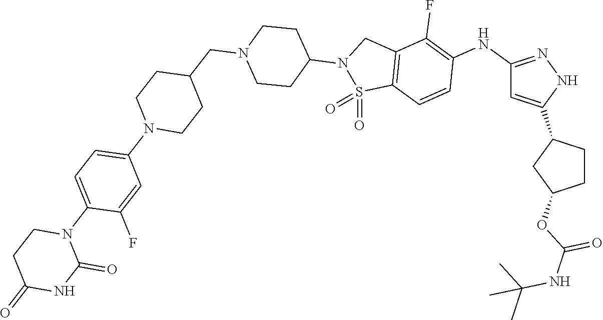 [SMILES: CC(C)(C)NC(=O)O[C@H]1CC[C@@H](c2cc(Nc3ccc4c(c3F)CN(C3CCN(CC5CCN(c6ccc(N7CCC(=O)NC7=O)c(F)c6)CC5)CC3)S4(=O)=O)n[nH]2)C1]

### Step 1: 3-((4-bromo-2-fluorophenyl)amino)propanoic acid

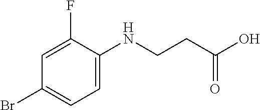 [SMILES: O=C(O)CCNc1ccc(Br)cc1F]

4-bromo-2-fluoroaniline (2.0 g, 9.8 mmol) and acrylic acid (1.41 g, 19.6 mmol) were dissolved in AcOH (20 mL) and H₂O (4 mL), the reaction mixture was stirred for 16 hours at 110° C. The resulting mixture was cooled to room temperature, concentrated under reduce pressure, and the residue was purified by column chromatography on silica gel PE/EA (1:1) to afford the product (2.65 g, 98%). LC-MS (ESI): m/z [M+H]⁺=262.1

### Step 2: 1-(4-bromo-2-fluorophenyl)dihydropyrimidine-2,4(1H,3H)-dione

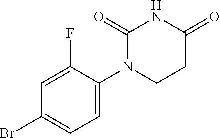 [SMILES: O=C1CCN(c2ccc(Br)cc2F)C(=O)N1]

3-((4-bromo-2-fluorophenyl)amino)propanoic acid (2 g, 7.63 mmol) was dissolved in AcOH (30 mL), urea (4.58 g, 76.33 mmol) was added, the reaction mixture was stirred for 16 hours at 110° C. The resulting mixture was cooled to room temperature, concentrated under reduce pressure. Then EA was added and the mixture wand washed with saturated Na₂CO₃ aqueous. The combined organic phase was washed with brine, dried over Na₂SO₄, filtered, the filtrated was concentrated under reduce pressure, the residue was purified by silica gel column chromatography, eluting with DCM/MeOH (20:1) to afford the product (1.5 g, 68.8% yield). LC-MS (ESI): m/z [M+H]⁺=286.2

### Step 3: 1-(4-(4-(dimethoxymethyl)piperidin-1-yl)-2-fluorophenyl)dihydropyrimidine-2,4(1H,3H)-dione

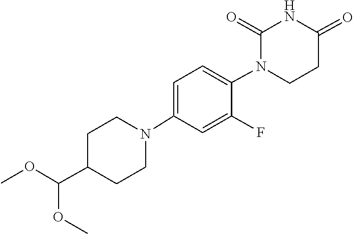 [SMILES: COC(OC)C1CCN(c2ccc(N3CCC(=O)NC3=O)c(F)c2)CC1]

To a solution of 1-(4-bromo-2-fluorophenyl)dihydropyrimidine-2,4(1H,3H)-dione (300 mg, 1.05 mmol) and 4-(dimethoxymethyl)piperidine (336 mg, 2.09 mmol) in dioxane (20 mL) was added Pd-PEPPSI-IPentCl 2-methylpyridine (44 mg, 0.05 mmol) and Cs₂CO₃ (1.02 g, 3.15 mmol), the reaction mixture was stirred for 16 hours at 100° C. under nitrogen. The resulting mixture was cooled to room temperature, filtered, the filtrate was concentrated under reduce pressure. The residue was purified by silica gel column chromatography, eluting with DCM/MeOH (20:1) to afford the product (110 mg, 28% yield). LC-MS (ESI): m/z [M+H]⁺=366.2.

### Step 4:1-(4-(2,4-dioxotetrahydropyrimidin-1(2H)-yl)-3-fluorophenyl)piperidine-4-carbaldehyde

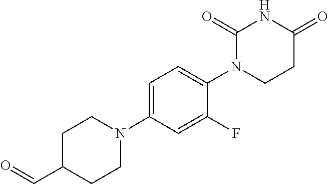 [SMILES: O=CC1CCN(c2ccc(N3CCC(=O)NC3=O)c(F)c2)CC1]

1-(4-(4-(dimethoxymethyl)piperidin-1-yl)-2-fluorophenyl)dihydropyrimidine-2,4(1H,3H)-dione (110 mg, 0.3 mmol) was dissolved in formic acid (5 mL), stirred for 2 hours at room temperature. The resulting mixture was concentrated under reduce pressure, and dried over in vacuum to afford the product (90 mg, 95%). LC-MS (ESI): m/z [M+H]⁺=319.4.

### Step 5: (1S,3R)-3-(3-((2-(1-((1-(4-(2,4-dioxotetrahydropyrimidin-1(2H)-yl)-3-fluorophenyl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

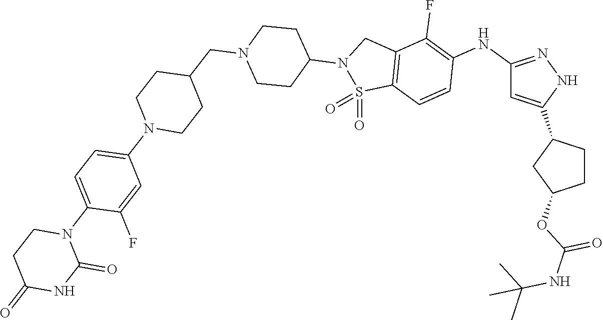 [SMILES: CC(C)(C)NC(=O)O[C@H]1CC[C@@H](c2cc(Nc3ccc4c(c3F)CN(C3CCN(CC5CCN(c6ccc(N7CCC(=O)NC7=O)c(F)c6)CC5)CC3)S4(=O)=O)n[nH]2)C1]

The titled compound was synthesized in the procedures similar to Example 19. ¹H NMR (500 MHz, DMSO) δ 11.99 (s, 1H), 10.37 (s, 1H), 8.79 (s, 1H), 8.30 (s, 1H), 7.52 (d, J=8.5 Hz, 1H), 7.19 (t, J=9.0 Hz, 1H), 6.79 IE (m, 3H), 5.83 (s, 1H), 4.98 (s, 1H), 4.53 (s, 2H), 3.76 (d, J=10.4 Hz, 2H), 3.66-3.57 (m, 3H), 3.42 (s, 1H), 3.05 (m, 2H), 2.91 (m, 1H), 2.79-2.66 (m, 5H), 2.54 (m, 2H), 2.45 (m, 1H), 2.08 (m, 2H), 2.06-1.99 (m, 2H), 1.98-1.83 (m, 4H), 1.79 (m, 3H), 1.70 (m, 3H), 1.58 (m, 1H), 1.21 (s, 10H). LC-MS (ESI): m/z [M+H]⁺=838.4

### Example 22: (1S,3R)-3-(3-((2-(1-((1-(4-(2,4-dioxotetrahydropyrimidin-1(2H)-yl)ph-enyl)azetidin-3-yl)methyl)piperidin-4-yl)-4-fluoro-1,1-dioxido-2,3-dihydrobenzo[d]isoth-iazol-5-yl)amino)-1H-pyrazol-5-yl)cyclopentyl tert-butylcarbamate

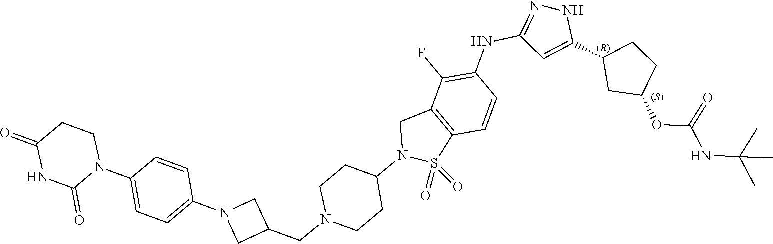

### Step 1: 4-(2,4-dioxo-3-((2-(trimethylsilyl)ethoxy)methyl)tetrahydropyrimidin-1(2H)-yl)phenyl trifluoromethanesulfonate

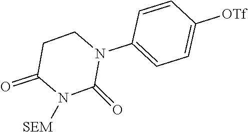 [SMILES: C[Si](C)(C)CCOCN1C(=O)CCN(c2ccc(OS(=O)(=O)C(F)(F)F)cc2)C1=O]

To a mixture of 1-(4-hydroxyphenyl)-3-((2-(trimethylsilyl)ethoxy)methyl)dihydropyr-imidine-2,4(1H,3H)-dione (5 g, 14.9 mmol) and Pyridine (2.35 g, 29.8 mmol) in DCM (100 mL) was added Tf₂O (5 g, 17.88 mmol) dropwise at 0° C. under nitrogen atmosphere. The reaction mixture was stirred for 5 hours at room temperature. The mixture was concentrated under reduced pressure. The residue was purified by silica gel column chromatography, eluting with PE/EA (2:1) to afford the product (5.6 g, 81% yield). LC-MS (ESI): m/z [M+H]⁺=469.1.

### Step 2: 1-(4-(3-(hydroxymethyl)azetidin-1-yl)phenyl)-3-((2-(trimethylsilyl)ethoxy)meth-yl)dihydropyrimidine-2,4(1H,3H)-dione

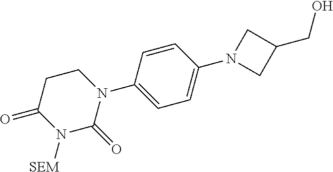 [SMILES: C[Si](C)(C)CCOCN1C(=O)CCN(c2ccc(N3CC(CO)C3)cc2)C1=O]

To a mixture of 4-(2,4-dioxo-3-((2-(trimethylsilyl)ethoxy)methyl)tetrahydropyrimidin-1(2H)-yl)phenyl trifluoromethanesulfonate (500 mg, 1.07 mmol) and azetidin-3-ylmethanol (140 mg, 1.60 mmol) in andydrous 1,4-dioxane (10 mL) was added Pd₂(dba)₃ (97.8 mg, 0.107 mmol), RuPhos (99.8 mg, 0.214 mmol) and Cs₂CO₃ (1.04 g, 3.20 mmol). The reaction mixture was stirred under nitrogen atmosphere at 100° C. for 16 h. The mixture was filtered, and the filtrate was concentrated under reduced pressure. The residue was purified by silica gel column chromatography, eluting with PE/EA (1:9) to afford the product (200 mg, 46% yield). LC-MS (ESI): m/z [M+H]⁺=406.2.

### Step 3: 1-(4-(2,4-dioxo-3-((2-(trimethylsilyl)ethoxy)methyl)tetrahydropyrimidin-1(2H)-yl)phenyl)azetidine-3-carbaldehyde

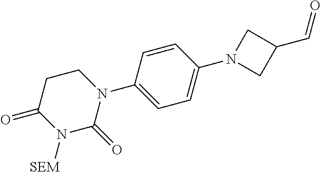 [SMILES: C[Si](C)(C)CCOCN1C(=O)CCN(c2ccc(N3CC(C=O)C3)cc2)C1=O]

To a stirred solution of 1-(4-(3-(hydroxymethyl)azetidin-1-yl)phenyl)-3-((2-(trimeth-ylsilyl)ethoxy)meth-yl)dihydropyrimidine-2,4(1H,3H)-dione (200 mg, 0.49 mmol) in DMSO (5 mL) was added IBX (276 mg, 0.99 mmol). The resulting solution was stirred at room temperature for 2 h. quenched with NaHCO₃ (2 mL) The reaction mixture was poured into water (20 ml) and extracted with EA (50 mL×3). The combined organic layers were dried over anhydrous sodium sulfate, filtrated, and concentrated under reduced pressure to afford the crude product (110 mg, 55% yield). LC-MS (ESI): m/z [M+H]⁺=404.3.

### Step 4: (1S,3R)-3-(3-((2-(1-((1-(4-(2,4-dioxo-3-((2-(trimethylsilyl)ethoxy)methyl)te-trahydropyrimidin-1(2H)-yl)phenyl)azetidin-3-yl)methyl)piperidin-4-yl)-4-fluoro-1,1-dio-xido-2,3-dihydrobenzo[d]isothiazol-5-yl)amino)-1H-pyrazol-5-yl)cyclopentyl tert-butyl-carbamate

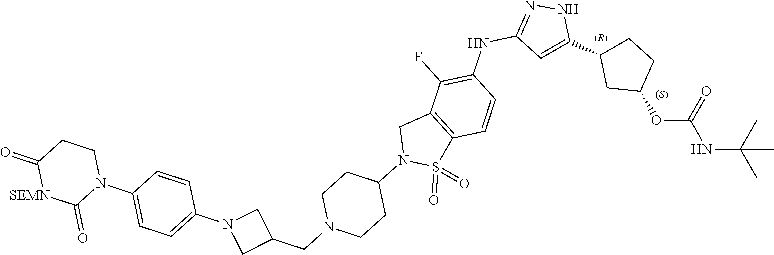

To a solution of (1S,3R)-3-(3-((4-fluoro-1,1-dioxido-2-(piperidin-4-yl)-2,3-dihydrobenz-o[d]isothiazol-5-yl)amino)-1H-pyrazol-5-yl)cyclopentyl tert-butyl carbamate (50 mg, 0.09 mmol) and 1-(4-(2,4-dioxo-3-((2-(trimethylsilyl)ethoxy)methyl)tetrahydropyrimidin-1(2H)-yl)phenyl)azetidine-3-carbaldehyde (57 mg, 0.14 mmol) in DMA (2 mL) was stirred for 30 min, then STAB (40 mg, 0.187 mmol) was added. the reaction mixture was stirred for 16 hours at room temperature. The resulting mixture was concentrated under reduce pressure, the residue was purified by column chromatography on silica gel, eluting with DCM/MeOH (10:1) to afford the product (40 mg, 46%). LC-MS (ESI): m/z [M+H]⁺=922.4.

### Step 4: (1S,3R)-3-(3-((2-(1-((1-(4-(2,4-dioxotetrahydropyrimidin-1(2H)-yl)phenyl)a-zetidin-3-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

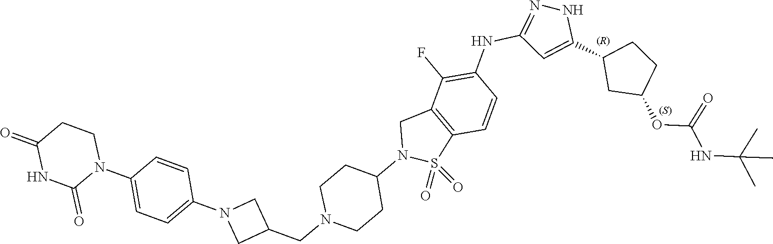

To a solution of (1S,3R)-3-(3-((2-(1-((1-(4-(2,4-dioxo-3-((2-(trimethylsilyl)ethoxy)m-ethyl)te-trahydropyrimidin-1(2H)-yl)phenyl)azetidin-3-yl)methyl)piperidin-4-yl)-4-fluoro-1,1-dio-xido-2,3-dihydrobenzo[d]isothiazol-5-yl)amino)-1H-pyrazol-5-yl)cyclopentyl tert-butyl-carbamate (40 mg, 0.043 mmol) in trifluoroacetic acid (4 mL). The reaction mixture was stirred for 1 hours at room temperature. The resulting mixture was concentrated, then the reaction mixture in THF (5 ml) was added NH₃—H₂O (28-30 wt %, 0.2 mL) dropwise. The reaction mixture was stirred for 10 min at room temperature. The mixture was concentrated under reduced pressure. The residue was purified by silica gel column chromatography, eluting with DCM/MeOH (10:1) to afford the product (20 mg, 59% yield). The residue 20 mg was purified by prep HPLC (Waters XSelect C18: RD-CO-094 column, eluting with a gradient of acetonitrile/water containing 0.1% FA, 20%-40%) to afford the product (5.0 mg, 15%). LC-MS (ESI): m/z [M+H]⁺=792.20. ¹H NMR (500 MHz, DMSO) δ 11.99 (s, 1H), 10.22 (s, J=6.6 Hz, 1H), 8.78 (s, 1H), 8.29 (s, 1H), 7.52 (d, J=8.6 Hz, 1H), 7.10 (d, J=8.6 Hz, 2H), 6.76 (s, 1H), 6.42 (d, J=8.7 Hz, 2H), 5.83 (s, 1H), 4.98 (s, 1H), 4.53 (d, J=10.5 Hz, 2H), 3.95 (t, J=6.6 Hz, 2H), 3.67 (d, J=6.7 Hz, 2H), 3.48 (s, 2H), 3.12-2.86 (m, 4H), 2.67 (t, J=6.7 Hz, 2H), 2.45 (d, J=7.8 Hz, 4H), 2.15-1.83 (m, 7H), 1.78-1.66 (m, 2H), 1.58 (s, 1H), 1.21 (s, 9H).

### Example 23: (1S,3R)-3-(3-((2-((1r,4r)-4-(((1-(4-(2,4-dioxotetrahydropyrimidin-1(2H)-yl)phenyl)piperidin-4-yl)methyl)(methyl)amino)cyclohexyl)-4-fluoro-1,1-dioxido-2,3-dihydrobenzo[d]isothiazol-5-yl)amino)-1H-pyrazol-5-yl)cyclopentyl tert-butylcarbamate

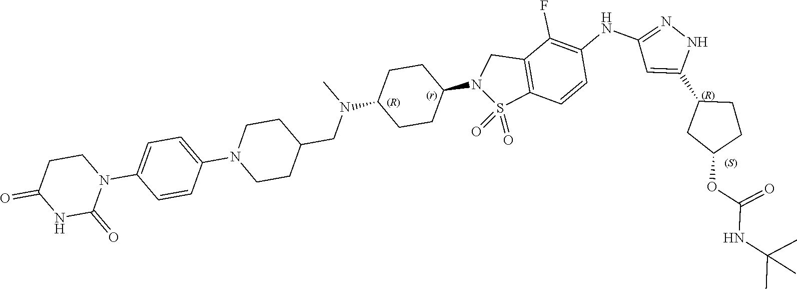

### Step 1: tert-butyl ((1r,4r)-4-(5-bromo-4-fluoro-1,1-dioxidobenzo[d]isothiazol-2(3H)-yl)cyclohexyl)(methyl)carbamate

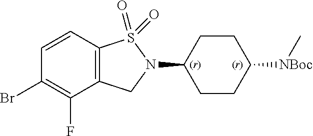

To a mixture of 5-bromo-4-fluoro-2,3-dihydrobenzo[d]isothiazole 1,1-dioxide (500 mg, 1.88 mmol) and tert-butyl (3R,4S)-3-fluoro-4-hydroxypiperidine-1-carboxylate (648 mg, 2.83 mmol) in toluene (10 mL) was added CMBP (1.36 g, 5.66 mmol). The reaction mixture was stirred under nitrogen atmosphere at 100° C. for 16 h. The mixture was concentrated under reduced pressure. The residue was purified by silica gel column chromatography, eluting with PE/EA (1:9) to afford the product (500 mg, 56% yield). LC-MS (ESI): m/z [M+H]⁺=477.3.

### Step 2: tert-butyl ((1R,4r)-4-(5-((5-((1R,3S)-3-((tert-butylcarbamoyl)oxy)cyclopentyl)-1H-pyrazol-3-yl)amino)-4-fluoro-1,1-dioxidobenzo[d]isothiazol-2(3H)-yl)cyclohexyl)(meth-yl)carbamate

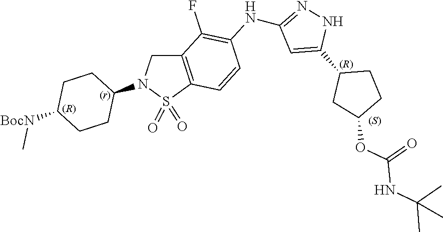

To a mixture of tert-butyl ((1r,4r)-4-(5-bromo-4-fluoro-1,1-dioxidobenzo[d]isothiazol-2(3H)-yl)cyclohexyl)(methyl)carbamate (400 mg, 0.84 mmol) and (1S,3R)-3-(3-amino-1H-pyrazol-5-yl)cyclopentyl tert-butylcarbamate (223 mg, 0.84 mmol) in t-BuOH (10 mL) was added Brettphos Pd G3 (36 mg, 0.084 mmol) and K₂CO₃ (348 mg, 2.52 mmol). The reaction mixture was stirred under nitrogen atmosphere at 110° C. for 16 h. The mixture was filtered, and the filtrate was concentrated under reduced pressure. The residue was purified by silica gel column chromatography, eluting with PE/EA (1:9) to afford the product (450 mg, 72% yield). LC-MS (ESI): m/z [M+H]⁺=663.3.

### Step 3: (1S,3R)-3-(3-((4-fluoro-2-((1r,4r)-4-(methylamino)cyclohexyl)-1,1-dioxido-2,3-dihydrobenzo[d]isothiazol-5-yl)amino)-1H-pyrazol-5-yl)cyclopentyl tert-butylcarbamate

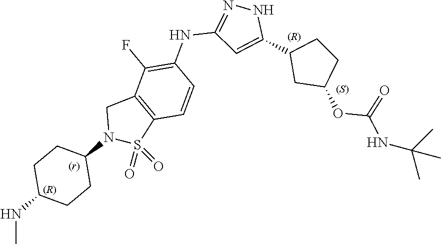

To a solution of tert-butyl ((1r,4r)-4-(5-((5-((1R,3S)-3-((tert-butylcarbamoyl)oxy)cy-clopentyl)-1H-pyrazol-3-yl)amino)-4-fluoro-1,1-dioxidobenzo[d]isothiazol-2(3H)-yl)cycloh-exyl)(meth-yl)carbamate (450 mg, 0.68 mmol) in dichloromethane (10 mL) was added trifluoroacetic acid (4 mL). The reaction mixture was stirred for 1 hours at room temperature. The resulting mixture was concentrated, dried over in vacuum, to afford the product (382 mg, 100% yield). LC-MS (ESI): m/z [M+H]⁺=563.3.

### Step 4: (1S,3R)-3-(3-((2-((1r,4r)-4-(((1-(4-(2,4-dioxotetrahydropyrimidin-1(2H)-yl)phenyl)piperidin-4-yl)methyl)(methyl)amino)cyclohexyl)-4-fluoro-1,1-dioxido-2,3-dihydrobenzo[d]isothiazol-5-yl)amino)-1H-pyrazol-5-yl)cyclopentyl tert-butylcarbamate

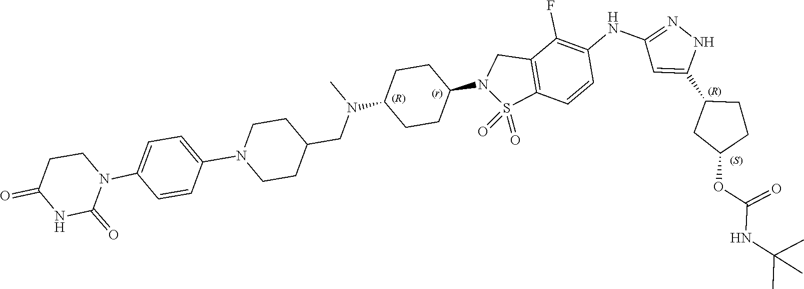

To a solution of (1S,3R)-3-(3-((4-fluoro-2-((1r,4R)-4-(methylamino)cyclohexyl)-1,1-dioxido-2,3-dihydrobenzo[d]isothiazol-5-yl)amino)-1H-pyrazol-5-yl)cyclopentyl tert-butyl carbamate (40 mg, 0.071 mmol) and 1-(4-(2,4-dioxotetrahydropyrimidin-1(2H)-yl)phenyl)-pip-eridine-4-carbaldehyde (32 mg, 0.106 mmol) in DMA (1.5 mL) was stirred for 30 min, then STAB (30 mg, 0.142 mmol) was added. the reaction mixture was stirred for 16 hours at room temperature. The resulting mixture was concentrated under reduce pressure, the residue was purified by column chromatography on silica gel, eluting with DCM/MeOH (10:1) to afford the product (40 mg). The residue 40 mg was purified by prep HPLC (Waters XSelect C18: RD-CO-094 column, eluting with a gradient of acetonitrile/water containing 0.1% FA, 20%-40%) to afford the product (15 mg, 25%). LC-MS (ESI): m/z [M+H]⁺=848.57. ¹H NMR (500 MHz, DMSO) δ 11.98 (s, 1H), 10.25 (s, 1H), 8.76 (s, 1H), 8.28 (s, 1H), 7.50 (d, J=8.6 Hz, 1H), 7.13 (d, J=8.9 Hz, 2H), 6.93 (d, J=9.0 Hz, 2H), 6.76 (s, 1H), 5.83 (s, 1H), 4.98 (s, 1H), 4.49 (s, 2H), 3.69 (t, J=6.7 Hz, 4H), 3.46 (t, J=11.5 Hz, 1H), 3.10-3.01 (m, 1H), 2.69-2.66 (m, 3H), 2.65-2.62 (m, 1H), 2.58-2.51 (m, 2H), 2.48-2.41 (m, 2H), 2.34-2.24 (m, 3H), 2.02-1.97 (m, 3H), 1.95-1.77 (m, 5H), 1.76-1.55 (m, 6H), 1.53-1.41 (m, 2H), 1.21 (s, 9H), 1.20-1.18 (m, 2H).

### Example 24: (1S,3R)-3-(3-((2-(1-(1-(4-(2,4-dioxotetrahydropyrimidin-1(2H)-yl)phenyl)piperidine-4-carbonyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

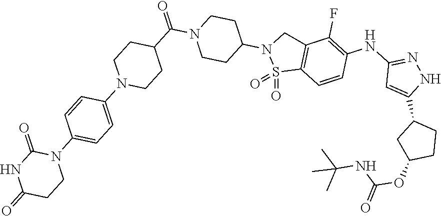 [SMILES: CC(C)(C)NC(=O)O[C@H]1CC[C@@H](c2cc(Nc3ccc4c(c3F)CN(C3CCN(C(=O)C5CCN(c6ccc(N7CCC(=O)NC7=O)cc6)CC5)CC3)S4(=O)=O)n[nH]2)C1]

### Step 1: tert-butyl 1-(4-(2,4-dioxo-3-((2-(trimethylsilyl)ethoxy)methyl) tetrahydropyrimidin-1(2H)-yl)phenyl)piperidine-4-carboxylate

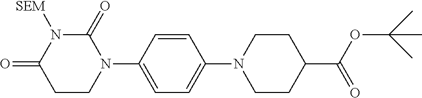 [SMILES: CC(C)(C)OC(=O)C1CCN(c2ccc(N3CCC(=O)N(COCC[Si](C)(C)C)C3=O)cc2)CC1]

To a solution of 2,6-bis(benzyloxy)-3-(4-bromo-3,5-difluorophenyl)pyridi 4-(2,4-dioxo-3-((2-(trimethylsilyl)ethoxy)methyl)tetrahydropyrimidin-1(2H)-yl)phenyl trifluoromethanesulfonatene (300 mg, 0.64 mmol) and tert-butyl piperidine-4-carboxylate (120 mg, 0.64 mmol) in dioxane (10 mL) was added Pd₂dba₃ (30 mg, 0.032 mmol), RuPhos (30 mg, 0.064 mmol) and Cs₂CO₃ (630 mg, 1.92 mmol), the reaction mixture was stirred for 16 hours at 100° C. under nitrogen. The resulting mixture was cooled to room temperature, filtered, the filtrate was concentrated under reduce pressure. The residue was purified by silica gel column chromatography, eluting with PE/EA (1:2) to afford the product (230 mg, 71% yield). LC-MS (ESI): m/z [M+H]⁺=504.4.

### Step 2: 1-(4-(2,4-dioxotetrahydropyrimidin-1(2H)-yl)phenyl)piperidine-4-carboxylic acid

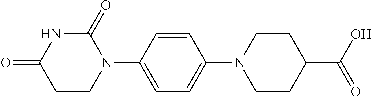 [SMILES: O=C1CCN(c2ccc(N3CCC(C(=O)O)CC3)cc2)C(=O)N1]

Tert-butyl 1-(4-(2,4-dioxo-3-((2-(trimethylsilyl)ethoxy)methyl) tetrahydropyrimidin-1(2H)-yl)phenyl)piperidine-4-carboxylate (230 mg, 0.46 mmol) was dissolved in DCM (5 mL), trifluoroacetic acid (1 mL) was added, the reaction mixture was stirred for 2 hours at room temperature. The mixture was concentrated under reduce pressure, redissolved in MeOH (5 mL), NH₄ in MeOH (7M, 1 mL) was added, stirred for 10 minutes. The resulting mixture was concentrated, dried over in vacuo, to afford the product (140 mg, 98% yield). LC-MS (ESI): m/z [M+H]⁺=318.2.

### Step 3: (1S,3R)-3-(3-((2-(1-(1-(4-(2,4-dioxotetrahydropyrimidin-1(2H)-yl)phenyl)piperidine-4-carbonyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

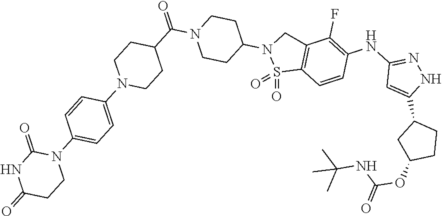 [SMILES: CC(C)(C)NC(=O)O[C@H]1CC[C@@H](c2cc(Nc3ccc4c(c3F)CN(C3CCN(C(=O)C5CCN(c6ccc(N7CCC(=O)NC7=O)cc6)CC5)CC3)S4(=O)=O)n[nH]2)C1]

The titled compound was synthesized in the procedures similar to Example 12. LC-MS (ESI): m/z [M+H]⁺=834.61; ¹H NMR (500 MHz, DMSO) δ=12.02 (s, 1H), 10.40 (s, 1H), 8.79 (s, 1H), 8.28 (t, J=7.9, 1H), 7.53 (d, J=8.7, 1H), 7.41 (s, 4H), 6.74 (s, 1H), 5.84 (s, 1H), 4.99 (s, 1H), 4.54 (s, 2H), 4.42 (d, J=12.5, 1H), 4.07 (d, J=12.6, 1H), 3.79 (m, 3H), 3.70 (m, 3H), 3.32-3.26 (m, 2H), 3.12-3.01 (m, 2H), 2.82 (t, J=11.6, 1H), 2.71 (m, 2H), 2.49-2.45 (m, 1H), 2.01 (m, 2H), 1.91 (m, 7H), 1.71 (m, 3H), 1.59 (m, 1H), 1.21 (s, 9H).

### Example 25: (1S,3R)-3-(3-((2-(cis-4-(4-(4-(2,4-dioxotetrahydropyrimidin-1(2H)-yl)phenyl)piperazin-1-yl)cyclohexyl)-4-fluoro-1,1-dioxido-2,3-dihydrobenzo[d]isothiazol-5-yl)amino)-1H-pyrazol-5-yl)cyclopentyl tert-butylcarbamate

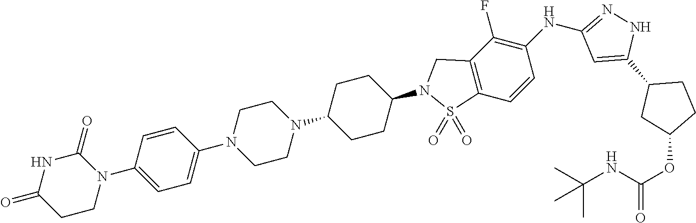 [SMILES: CC(C)(C)NC(=O)O[C@H]1CC[C@@H](c2cc(Nc3ccc4c(c3F)CN([C@H]3CC[C@H](N5CCN(c6ccc(N7CCC(=O)NC7=O)cc6)CC5)CC3)S4(=O)=O)n[nH]2)C1]

The titled compound was synthesized in the procedures similar to Example 2. LC-MS (ESI): m/z [M+H]⁺=806.70; ¹H NMR (500 MHz, DMSO) δ=11.98 (s, 1H), 10.25 (s, 1H), 8.76 (s, 1H), 8.30 (t, J=8.1, 1H), 7.51 (d, J=8.6, 1H), 7.15 (d, J=8.9, 2H), 6.93 (d, J=9.1, 2H), 6.76 (s, 1H), 5.83 (s, 1H), 4.98 (s, 1H), 4.50 (s, 2H), 3.70 (t, J=6.7, 2H), 3.45 (m, 1H), 3.12 (m, 4H), 3.07-3.03 (m, 1H), 2.67 (m, 5H), 2.47-2.43 (m, 1H), 2.32 (m, 1H), 2.01 (m, 3H), 1.93 (m, 3H), 1.69 (m, 4H), 1.58 (m, 1H), 1.41 (m, 2H), 1.21 (s, 9H).

### Example 26: (1S,3R)-3-(3-((2-(1′-(1-(4-(2,4-dioxotetrahydropyrimidin-1(2H)-yl)phenyl)azetidin-3-yl)-[1,4′-bipiperidin]-4-yl)-4-fluoro-1,1-dioxido-2,3-dihydrobenzo[d]isothiazol-5-yl)amino)-1H-pyrazol-5-yl)cyclopentyl tert-butylcarbamate

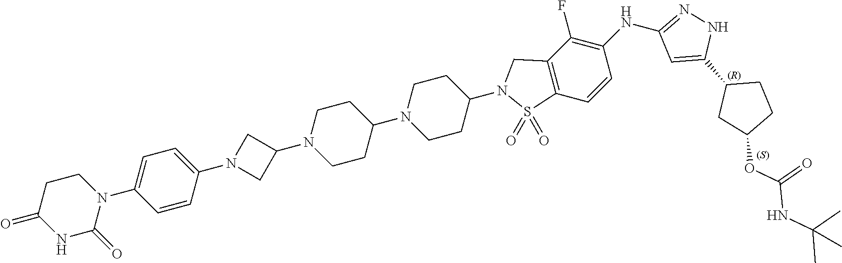

The titled compound was synthesized in the procedures similar to Example 19. ¹H NMR (500 MHz, DMSO) δ 11.99 (s, 1H), 10.23 (s, 1H), 8.79 (s, 1H), 8.30 (s, 1H), 7.52 (d, J=8.7 Hz, 1H), 7.11 (d, J=8.7 Hz, 2H), 6.75 (s, 1H), 6.43 (d, J=8.7 Hz, 2H), 5.83 (s, 1H), 4.97 (s, 1H), 4.53 (s, 2H), 3.93 (t, J=6.8 Hz, 2H), 3.67 (t, J=6.7 Hz, 2H), 3.56 (s, 2H), 3.11-3.01 (m, 2H), 2.92 (s, 2H), 2.67 (t, J=6.7 Hz, 2H), 2.64 (dt, J=3.7, 1.8 Hz, 1H), 2.56-2.51 (m, 2H), 2.48-2.42 (m, 2H), 2.36 (dt, J=3.6, 1.8 Hz, 1H), 2.25-1.82 (m, 10H), 1.70 (d, J=16.1, 8.4 Hz, 2H), 1.58 (s, 3H), 1.21 (s, 9H). LC-MS (ESI): m/z [M+H]⁺=861.65

### Example 27a and 27b: (1S,3R)-3-(3-((2-((S)-1-((1-(4-(2,4-dioxotetrahydropyrimidin-1(2H)-yl)phenyl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl tert-butylcarbamate and

### (1S,3R)-3-(3-((2-((R)-1-((1-(4-(2,4-dioxotetrahydropyrimidin-1(2H)-yl)phenyl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl tert-butylcarbamate

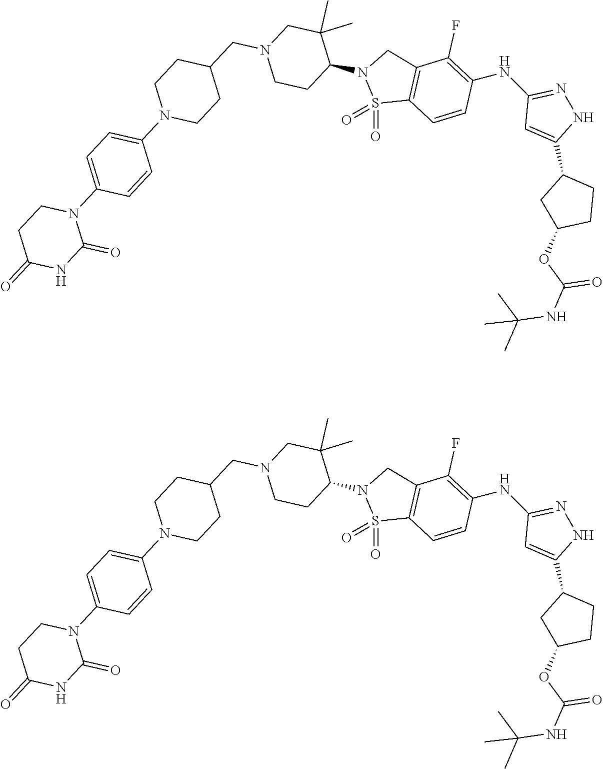 [SMILES: CC(C)(C)NC(=O)O[C@H]1CC[C@@H](c2cc(Nc3ccc4c(c3F)CN([C@H]3CCN(CC5CCN(c6ccc(N7CCC(=O)NC7=O)cc6)CC5)CC3(C)C)S4(=O)=O)n[nH]2)C1 ; CC(C)(C)NC(=O)O[C@H]1CC[C@@H](c2cc(Nc3ccc4c(c3F)CN([C@@H]3CCN(CC5CCN(c6ccc(N7CCC(=O)NC7=O)cc6)CC5)CC3(C)C)S4(=O)=O)n[nH]2)C1]

The titled compound was synthesized in the procedures similar to Example 19 in a racemic form, which was further separated by Chiral Prep-HPLC to give:

Enantiomer 1 (Example 27a, 100% ee); Retention time: 5.087 min. ¹H NMR (500 MHz, DMSO) δ 11.98 (s, 1H), 10.25 (s, 1H), 8.77 (s, 1H), 8.30 (d, J=7.1 Hz, 1H), 7.54 (d, J=8.6 Hz, 1H), 7.13 (d, J=8.9 Hz, 2H), 6.93 (d, J=9.0 Hz, 2H), 6.76 (s, 1H), 5.83 (s, 1H), 5.06-4.96 (m, 1H), 4.54 (s, 2H), 3.69 (t, J=6.7 Hz, 4H), 3.22 (dd, J=12.3, 3.7 Hz, 1H), 3.12-3.00 (m, 1H), 2.95 (d, J=9.4 Hz, 1H), 2.72-2.62 (m, 4H), 2.47-2.41 (m, 1H), 2.24-2.07 (m, 3H), 2.05-1.87 (m, 3H), 1.84-1.47 (m, 9H), 1.26-1.17 (m, 11H), 1.16-1.1 (m, 3H), 0.99-0.85 (m, 3H). LC-MS (ESI): m/z [M+H]⁺=848.4;

Enantiomer 1 (Example 27b, 100% ee); Retention time: 7.199 min. ¹H NMR (500 MHz, DMSO) δ 11.98 (s, 1H), 10.25 (s, 1H), 8.77 (s, 1H), 8.30 (d, J=7.1 Hz, 1H), 7.54 (d, J=8.6 Hz, 1H), 7.13 (d, J=8.9 Hz, 2H), 6.93 (d, J=9.0 Hz, 2H), 6.76 (s, 1H), 5.83 (s, 1H), 5.06-4.96 (m, 1H), 4.54 (s, 2H), 3.69 (t, J=6.7 Hz, 4H), 3.22 (dd, J=12.3, 3.7 Hz, 1H), 3.12-3.00 (m, 1H), 2.95 (d, J=9.4 Hz, 1H), 2.72-2.62 (m, 4H), 2.47-2.41 (m, 1H), 2.24-2.07 (m, 3H), 2.05-1.87 (m, 3H), 1.84-1.47 (m, 9H), 1.26-1.17 (m, 11H), 1.16-1.1 (m, 3H), 0.98-0.83 (m, 3H). LC-MS (ESI): m/z [M+H]⁺=848.4;

Chiral analytical method: Column: ICHIRALPAK SA 4.6*150 mm 5 μm; Mobile phase: A for MtBE (0.1%2M NH3 MeOH) (%) and B for MeOH:DCM=50:50(%); Gradient: Mobile Phase A: Mobile Phase B=20:80 (v/v); HPLC Equipment: HPLC-Agilent, Back pressure: 100 bar; Column temperature: 35° C.

Chiral Prep-HPLC Condition: CHIRALPAK SA 250*20.0 mml, 5 μm; Mobile phase: A for MtBE (0.1%2M NH3 MeOH) (%) and B for MeOH:DCM=50:50; Gradient: Mobile Phase A: Mobile Phase B=20:80 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 28: (1S,3R)-3-(3-((2-((3R,4R)-1-((1-(4-(2,4-dioxotetrahydropyrimidin-1(2H)yl)phenyl)piperidin-4-yl)methyl)-3-fluoropiperidin-4-yl)-4-fluoro-1,1-dioxido-2,3-dihydrobenzo[d]isothiazol-5-yl)amino)-1H-pyrazol-5-yl)cyclopentyl tert-butylcarbamate

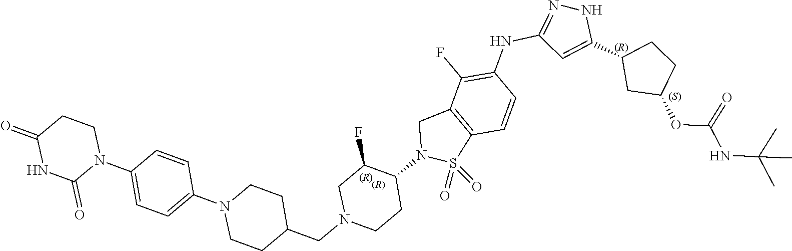

The titled compound was synthesized in the procedures similar to Example 19. ¹H NMR (500 MHz, DMSO) δ 11.99 (s, 1H), 10.25 (s, 1H), 8.79 (d, J=1.8 Hz, 1H), 8.31 (s, 1H), 7.54 (d, J=8.6 Hz, 1H), 7.13 (d, J=8.9 Hz, 2H), 6.93 (d, J=9.0 Hz, 2H), 6.76 (s, 1H), 5.84 (s, 1H), 4.98 (s, 1H), 4.89-4.72 (m, 1H), 4.68-4.58 (m, 2H), 3.73-3.56 (m, 5H), 3.28-3.21 (m, 1H), 3.11-3.02 (m, 1H), 2.85 (d, J=10.4 Hz, 1H), 2.71-2.62 (m, 4H), 2.48-2.43 (m, 1H), 2.31-2.22 (m, 2H), 2.17-1.85 (m, 6H), 1.83-1.63 (m, 5H), 1.59 (s, 1H), 1.21 (s, 11H). LC-MS (ESI): m/z [M+H]⁺=838.68

### Example 29: (1S,3R)-3-(3-((2-((3S,4S)-1-((1-(4-(2,4-dioxotetrahydropyrimidin-1(2H)yl)phenyl)piperidin-4-yl)methyl)-3-fluoropiperidin-4-yl)-4-fluoro-1,1-dioxido-2,3-dihydrobenzo[d]isothiazol-5-yl)amino)-1H-pyrazol-5-yl)cyclopentyl tert-butylcarbamate

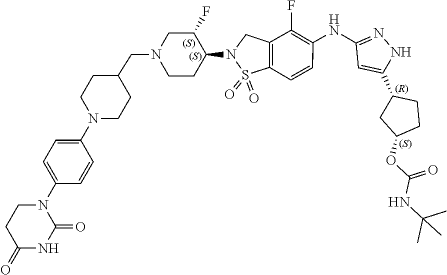

The titled compound was synthesized in the procedures similar to Example 19. ¹H NMR (500 MHz, DMSO) δ 11.99 (s, 1H), 10.25 (s, 1H), 8.79 (d, J=1.8 Hz, 1H), 8.31 (s, 1H), 7.54 (d, J=8.6 Hz, 1H), 7.13 (d, J=8.9 Hz, 2H), 6.93 (d, J=9.0 Hz, 2H), 6.76 (s, 1H), 5.84 (s, 1H), 4.98 (s, 1H), 4.89-4.72 (m, 1H), 4.68-4.58 (m, 2H), 3.73-3.56 (m, 5H), 3.28-3.21 (m, 1H), 3.11-3.02 (m, 1H), 2.85 (d, J=10.4 Hz, 1H), 2.71-2.62 (m, 4H), 2.48-2.43 (m, 1H), 2.31-2.22 (m, 2H), 2.17-1.85 (m, 6H), 1.83-1.63 (m, 5H), 1.59 (s, 1H), 1.21 (s, 11H). LC-MS (ESI): m/z [M+H]⁺=838.81

### Example 30: (1S,3R)-3-(3-((2-((3R,4S)-1-((1-(4-(2,4-dioxotetrahydropyrimidin-1(2H)yl)phenyl)piperidin-4-yl)methyl)-3-fluoropiperidin-4-yl)-4-fluoro-1,1-dioxido-2,3-dihydrobenzo[d]isothiazol-5-yl)amino)-1H-pyrazol-5-yl)cyclopentyl tert-butylcarbamate

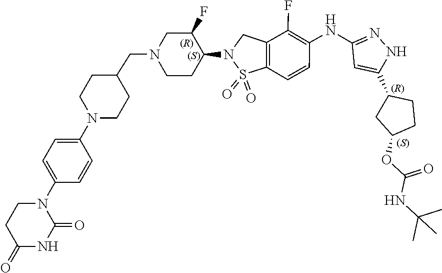

The titled compound was synthesized in the procedures similar to Example 19. ¹H NMR (500 MHz, DMSO) δ 11.99 (s, 1H), 10.25 (s, 1H), 8.80 (s, 1H), 8.31 (s, 1H), 7.56 (d, J=8.7 Hz, 1H), 7.13 (d, J=8.9 Hz, 2H), 6.93 (d, J=9.0 Hz, 2H), 6.76 (s, 1H), 5.84 (s, 1H), 5.03-4.88 (m, 2H), 4.60 (dd, J=48.6, 14.0 Hz, 2H), 3.77-3.63 (m, 5H), 3.20-3.03 (m, 2H), 2.96 (d, J=9.1 Hz, 1H), 2.70-2.61 (m, 4H), 2.35 (d, J=13.0 Hz, 1H), 2.29-2.14 (m, 4H), 2.02 (dd, J=15.5, 7.6 Hz, 1H), 1.96-1.86 (m, 1H), 1.84-1.54 (m, 7H), 1.21 (s, 11H). LC-MS (ESI): m/z [M+H]⁺=838.76

### Example 31a and 31b and 31c and 31d: (1S,3R)-3-(3-((2-((3R,4S)-1-((1-(4-(2,4-dioxotetrahydropyrimidin-1(2H)-yl)phenyl)piperidin-4-yl)methyl)-3-methylpiperidin-4-yl)-4-fluoro-1,1-dioxido-2,3-dihydrobenzo[d]isothiazol-5-yl)amino)-1H-pyrazol-5-yl)cyclopentyl tert-butylcarbamate and

### (1S,3R)-3-(3-((2-((3S,4S)-1-((1-(4-(2,4-dioxotetrahydropyrimidin-1(2H)-yl)phenyl)piperidin-4-yl)methyl)-3-methylpiperidin-4-yl)-4-fluoro-1,1-dioxido-2,3-dihydrobenzo[d]isothiazol-5-yl)amino)-1H-pyrazol-5-yl)cyclopentyl tert-butylcarbamate

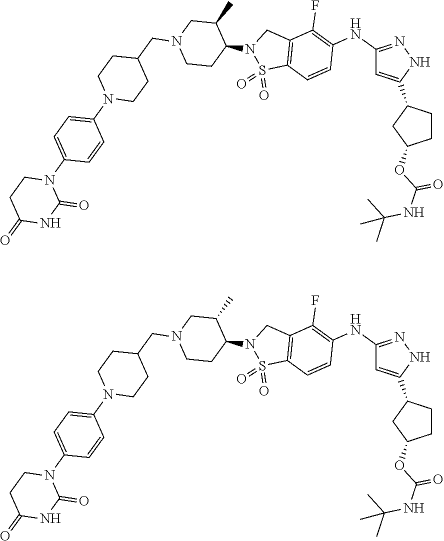 [SMILES: C[C@@H]1CN(CC2CCN(c3ccc(N4CCC(=O)NC4=O)cc3)CC2)CC[C@@H]1N1Cc2c(ccc(Nc3cc([C@@H]4CC[C@H](OC(=O)NC(C)(C)C)C4)[nH]n3)c2F)S1(=O)=O ; C[C@H]1CN(CC2CCN(c3ccc(N4CCC(=O)NC4=O)cc3)CC2)CC[C@@H]1N1Cc2c(ccc(Nc3cc([C@@H]4CC[C@H](OC(=O)NC(C)(C)C)C4)[nH]n3)c2F)S1(=O)=O]

The titled compound was synthesized in the procedures similar to Example 19 in a racemic form, which was further separated by Chiral Prep-HPLC to give:

Enantiomer 1 (Example 31a, 100% ee); Retention time: 7.949 min. ¹H NMR (500 MHz, DMSO) δ 11.98 (s, 1H), 10.25 (s, 1H), 8.76 (s, 1H), 8.30 (d, J=7.7 Hz, 1H), 7.52 (d, J=8.6 Hz, 1H), 7.13 (d, J=9.0 Hz, 2H), 6.92 (d, J=9.1 Hz, 2H), 6.76 (s, 1H), 5.83 (s, 1H), 5.06-4.96 (m, 1H), 4.55 (d, J=14.2 Hz, 1H), 4.43 (d, J=14.3 Hz, 1H), 3.72-3.65 (m, 4H), 3.15-3.05 (m, 2H), 2.96-2.86 (m, 2H), 2.72-2.61 (m, 5H), 2.17 (d, J=7.1 Hz, 2H), 2.05-1.87 (m, 5H), 1.85-1.51 (m, 8H), 1.21 (s, 11H), 0.95-0.81 (m, 3H). LC-MS (ESI): m/z [M+H]⁺=834.4;

Enantiomer 1 (Example 31b, 100% ee); Retention time: 9.306 min. ¹H NMR (500 MHz, DMSO) δ 11.98 (s, 1H), 10.25 (s, 1H), 8.76 (s, 1H), 8.30 (d, J=7.7 Hz, 1H), 7.52 (d, J=8.6 Hz, 1H), 7.13 (d, J=9.0 Hz, 2H), 6.92 (d, J=9.1 Hz, 2H), 6.76 (s, 1H), 5.83 (s, 1H), 5.06-4.96 (m, 1H), 4.55 (d, J=14.2 Hz, 1H), 4.43 (d, J=14.3 Hz, 1H), 3.72-3.65 (m, 4H), 3.15-3.05 (m, 2H), 2.96-2.86 (m, 2H), 2.72-2.61 (m, 5H), 2.17 (d, J=7.1 Hz, 2H), 2.05-1.87 (m, 5H), 1.85-1.51 (m, 8H), 1.21 (s, 11H), 0.95-0.81 (m, 3H). LC-MS (ESI): m/z [M+H]⁺=834.4;

Enantiomer 1 (Example 31c, 100% ee); Retention time: 9.505 min. ¹H NMR (500 MHz, DMSO) δ 11.98 (s, 1H), 10.25 (s, 1H), 8.76 (s, 1H), 8.30 (d, J=7.7 Hz, 1H), 7.52 (d, J=8.6 Hz, 1H), 7.13 (d, J=9.0 Hz, 2H), 6.92 (d, J=9.1 Hz, 2H), 6.76 (s, 1H), 5.83 (s, 1H), 5.06-4.96 (m, 1H), 4.55 (d, J=14.2 Hz, 1H), 4.43 (d, J=14.3 Hz, 1H), 3.72-3.65 (m, 4H), 3.15-3.05 (m, 2H), 2.96-2.86 (m, 2H), 2.72-2.61 (m, 5H), 2.17 (d, J=7.1 Hz, 2H), 2.05-1.87 (m, 5H), 1.85-1.51 (m, 8H), 1.21 (s, 11H), 0.95-0.81 (m, 3H). LC-MS (ESI): m/z [M+H]⁺=834.4;

Enantiomer 1 (Example 31d, 100% ee); Retention time: 11.509 min. ¹H NMR (500 MHz, DMSO) δ 11.98 (s, 1H), 10.25 (s, 1H), 8.76 (s, 1H), 8.30 (d, J=7.7 Hz, 1H), 7.52 (d, J=8.6 Hz, 1H), 7.13 (d, J=9.0 Hz, 2H), 6.92 (d, J=9.1 Hz, 2H), 6.76 (s, 1H), 5.83 (s, 1H), 5.06-4.96 (m, 1H), 4.55 (d, J=14.2 Hz, 1H), 4.43 (d, J=14.3 Hz, 1H), 3.72-3.65 (m, 4H), 3.15-3.05 (m, 2H), 2.96-2.86 (m, 2H), 2.72-2.61 (m, 5H), 2.17 (d, J=7.1 Hz, 2H), 2.05-1.87 (m, 5H), 1.85-1.51 (m, 8H), 1.21 (s, 11H), 0.95-0.81 (m, 3H). LC-MS (ESI): m/z [M+H]⁺=834.4;

Chiral analytical method: Column: ICHIRALPAK SA 4.6*150 mm 5 μm; Mobile phase: A for MtBE (0.1%2M NH3 MeOH) (%) and B for MeOH:DCM=50:50(%); Gradient: Mobile Phase A: Mobile Phase B=50:50 (v/v); HPLC Equipment: HPLC-Agilent, Back pressure: 100 bar; Column temperature: 35° C.

Chiral Prep-HPLC Condition: CHIRALPAK SA 250*20.0 mml, 5 μm; Mobile phase: A for MtBE (0.1%2M NH3 MeOH) (%) and B for MeOH:DCM=50:50; Gradient: Mobile Phase A: Mobile Phase B=50:50 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 32a and 32b: (1S,3R)-3-(3-((2-(1-(((R)-1-(4-(2,4-dioxotetrahydropyrimidin-1(2H)-yl)phenyl)-3,3-difluoropiperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate and (1S,3R)-3-(3-((2-(1-(((S)-1-(4-(2,4-dioxotetrahydropyrimidin-1(2H)-yl)phenyl)-3,3-difluoropiperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

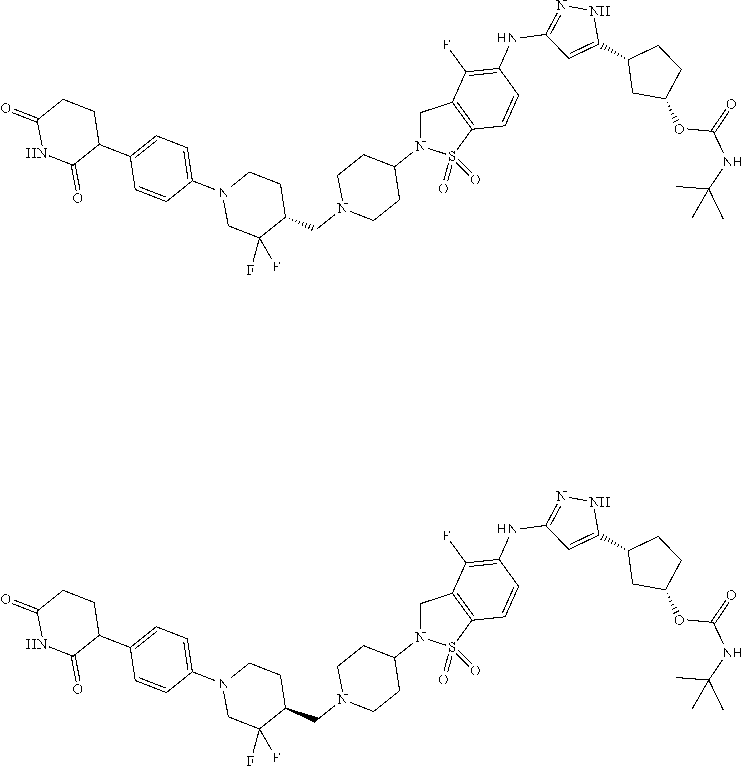 [SMILES: CC(C)(C)NC(=O)O[C@H]1CC[C@@H](c2cc(Nc3ccc4c(c3F)CN(C3CCN(C[C@H]5CCN(c6ccc(C7CCC(=O)NC7=O)cc6)CC5(F)F)CC3)S4(=O)=O)n[nH]2)C1 ; CC(C)(C)NC(=O)O[C@H]1CC[C@@H](c2cc(Nc3ccc4c(c3F)CN(C3CCN(C[C@@H]5CCN(c6ccc(C7CCC(=O)NC7=O)cc6)CC5(F)F)CC3)S4(=O)=O)n[nH]2)C1]

### Step 1: 1-(4-(3,3-difluoro-4-(hydroxymethyl)piperidin-1-yl)phenyl)-3-((2-(trimethylsilyl)ethoxy)methyl)dihydropyrimidine-2,4(1H,3H)-dione

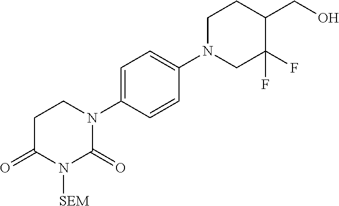 [SMILES: C[Si](C)(C)CCOCN1C(=O)CCN(c2ccc(N3CCC(CO)C(F)(F)C3)cc2)C1=O]

To a solution of 4-(2,4-dioxo-3-((2-(trimethylsilyl)ethoxy)methyl)tetrahydropyrimidin-1(2H)-yl)phenyl trifluoromethanesulfonate (1.2 g, 2.56 mmol) and (3,3-difluoropiperidin-4-yl)methanol (580 mg, 3.84 mmol) in dioxane (10 mL) was added Pd₂dba₃ (30 mg, 0.032 mmol), RuPhos (30 mg, 0.064 mmol) and Cs₂CO₃ (630 mg, 1.92 mmol), the reaction mixture was stirred for 16 hours at 100° C. under nitrogen. The resulting mixture was cooled to room temperature, filtered, the filtrate was concentrated under reduce pressure. The residue was purified by silica gel column chromatography, eluting with PE/EA (1:2) to afford the product (350 mg, 30% yield). LC-MS (ESI): m/z [M+H]⁺=470.3.

### Step 2: (1-(4-(2,4-dioxo-3-((2-(trimethylsilyl)ethoxy)methyl)tetrahydropyrimidin-1(2H)-yl)phenyl)-3,3-difluoropiperidin-4-yl)methyl methanesulfonate

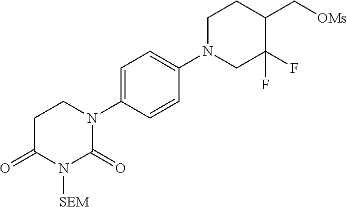 [SMILES: C[Si](C)(C)CCOCN1C(=O)CCN(c2ccc(N3CCC(COS(C)(=O)=O)C(F)(F)C3)cc2)C1=O]

To a solution of 1-(4-(3,3-difluoro-4-(hydroxymethyl)piperidin-1-yl)phenyl)-3-((2-(trimethylsilyl)ethoxy)methyl)dihydropyrimidine-2,4(1H,3H)-dione (100 mg, 0.21 mmol) in dichloromethane (10 mL) was added TEA (43 mg, 0.42 mmol), MsCl (44 mg, 0.31 mmol) the reaction mixture was stirred for 2 hour at room temperature. The resulting mixture was concentrated under reduce pressure, The residue was purified by silica gel column chromatography, eluting with PE/EA (1:1) to afford the product (100 mg, 85.4% yield). LC-MS (ESI): m/z [M+H]⁺=548.2.

### Step 3: (1S,3R)-3-(3-((2-(1-(((R)-1-(4-(2,4-dioxotetrahydropyrimidin-1(2H)-yl)phenyl)-3,3-difluoropiperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate and (1S,3R)-3-(3-((2-(1-(((S-1-(4-(2,4-dioxotetrahydropyrimidin-1(2H)-yl)phenyl)-3,3-difluoropiperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

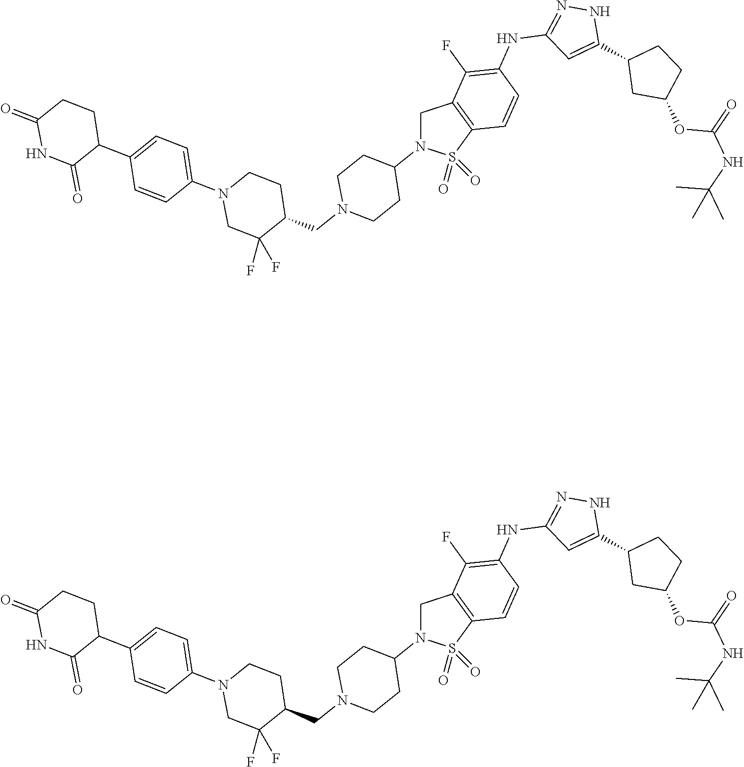 [SMILES: CC(C)(C)NC(=O)O[C@H]1CC[C@@H](c2cc(Nc3ccc4c(c3F)CN(C3CCN(C[C@H]5CCN(c6ccc(C7CCC(=O)NC7=O)cc6)CC5(F)F)CC3)S4(=O)=O)n[nH]2)C1 ; CC(C)(C)NC(=O)O[C@H]1CC[C@@H](c2cc(Nc3ccc4c(c3F)CN(C3CCN(C[C@@H]5CCN(c6ccc(C7CCC(=O)NC7=O)cc6)CC5(F)F)CC3)S4(=O)=O)n[nH]2)C1]

The titled compound was synthesized in the procedures similar to Example 19 in a racemic form, which was further separated by Chiral Prep-HPLC to give:

Enantiomer 1 (Example 32a, 100% ee); Retention time: 7.682 min. ¹H NMR (500 MHz, DMSO) δ 11.98 (s, 1H), 10.23 (s, 1H), 8.76 (s, 1H), 8.30 (t, J=7.9 Hz, 1H), 7.51 (d, J=8.7 Hz, 1H), 7.15 (d, J=8.9 Hz, 2H), 6.76 (s, 1H), 6.59 (d, J=9.0 Hz, 2H), 5.83 (s, 1H), 5.06-4.96 (m, 1H), 4.52 (s, 2H), 3.84-3.65-3.53 (m, 5H), 3.48-3.39 (m, 1H), 3.12-3.02 (m, 2H), 2.98-2.90 (m, 2H), 2.86-2.72 (m, 1H), 2.68 (t, J=6.7 Hz, 2H), 2.45-2.39 (m, 2H), 2.13-1.98 (m, 3H), 1.96-1.80 (m, 6H), 1.76-1.54 (m, 4H), 1.20 (d, J=10.1 Hz, 9H). LC-MS (ESI): m/z [M+H]⁺=856.4;

Enantiomer 3 (Example 32b, 100% ee); Retention time: 8.287 min. ¹H NMR (500 MHz, DMSO) δ 11.98 (s, 1H), 10.23 (s, 1H), 8.76 (s, 1H), 8.30 (t, J=7.9 Hz, 1H), 7.51 (d, J=8.7 Hz, 1H), 7.15 (d, J=8.9 Hz, 2H), 6.76 (s, 1H), 6.59 (d, J=9.0 Hz, 2H), 5.83 (s, 1H), 5.06-4.96 (m, 1H), 4.52 (s, 2H), 3.84-3.65-3.53 (m, 5H), 3.48-3.39 (m, 1H), 3.12-3.02 (m, 2H), 2.98-2.90 (m, 2H), 2.86-2.72 (m, 1H), 2.68 (t, J=6.7 Hz, 2H), 2.45-2.39 (m, 2H), 2.13-1.98 (m, 3H), 1.96-1.80 (m, 6H), 1.76-1.54 (m, 4H), 1.20 (d, J=10.1 Hz, 9H). LC-MS (ESI): m/z [M+H]⁺=856.4;

Chiral analytical method: Column: ICHIRALPAK SA 4.6*150 mm 5 μm; Mobile phase: A for MtBE (0.1%2M NH3 MeOH) (%) and B for MeOH:DCM=50:50(%); Gradient: Mobile Phase A: Mobile Phase B=20:80 (v/v); HPLC Equipment: HPLC-Agilent, Back pressure: 100 bar; Column temperature: 35° C.

Chiral Prep-HPLC Condition: CHIRALPAK SA 250*20.0 mml, 5 μm; Mobile phase: A for MtBE (0.1%2M NH3 MeOH) (%) and B for MeOH:DCM=50:50; Gradient: Mobile Phase A: Mobile Phase B=20:80 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 33a and 33b: (1S,3R)-3-(3-((2-((S)-1-((1-(4-(2,4-dioxotetrahydropyrimidin-1(2H)-yl)phenyl)piperidin-4-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl tert-butylcarbamate and (1S,3R)-3-(3-((2-((R)-1-((1-(4-(2,4-dioxotetrahydropyrimidin-1(2H)-yl)phenyl)piperidin-4-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl tert-butylcarbamate

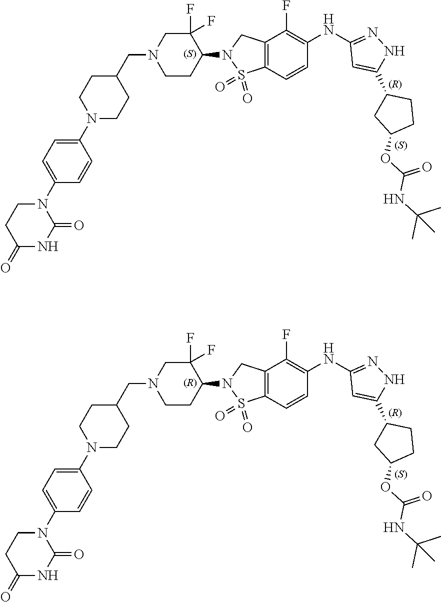

To a solution of (1S,3R)-3-(3-((2-(3,3-difluoropiperidin-4-yl)-4-fluoro-1,1-dioxido-2,3-dihydrobenzo[d]isothiazol-5-yl)amino)-1H-pyrazol-5-yl)cyclopentyl tert-butyl carbamate (60 mg, 0.105 mmol) and 1-(4-(2,4-dioxotetrahydropyrimidin-1(2H)-yl)phenyl)pip-eridine-4-carbaldehyde (53 mg, 0.158 mmol) in DMA (3 mL) was stirred for 30 min, then STAB (45 mg, 0.21 mmol) was added. the reaction mixture was stirred for 16 hours at room temperature. The resulting mixture was concentrated under reduce pressure, the residue was purified by column chromatography on silica gel, eluting with DCM/MeOH (10:1) to afford the product (35 mg, 37%) in a racemic form, which was further separated by Chiral Prep-HPLC to give:

Enantiomer 1 (example 33a, 100% ee); Retention time: 8.636 min. ¹H NMR (500 MHz, DMSO) δ 12.00 (s, 1H), 10.25 (s, 1H), 8.83 (s, 1H), 8.34 (t, J=7.6 Hz, 1H), 7.58 (d, J=8.7 Hz, 1H), 7.13 (d, J=8.9 Hz, 2H), 6.93 (d, J=9.0 Hz, 2H), 6.76 (s, 1H), 5.84 (s, 1H), 4.98 (s, 1H), 4.65 (dd, J=105.3, 13.7 Hz, 2H), 4.05-3.94 (m, 1H), 3.74-3.62 (m, 4H), 3.17 (t, J=9.8 Hz, 1H), 3.10-3.02 (m, 1H), 2.98-2.90 (m, 1H), 2.72-2.62 (m, 4H), 2.56 (m, J=11.8 Hz, 1H), 2.48-2.44 (m, 1H), 2.37-2.25 (m, 3H), 2.20 (dt, J=21.3, 10.6 Hz, 1H), 2.06-1.98 (m, 1H), 1.94-1.87 (m, 1H), 1.84-1.64 (m, 6H), 1.59 (s, 1H), 1.21 (s, 11H). LC-MS (ESI): m/z [M+H]⁺=856.72.

Enantiomer 2 (example 33b, 100% ee); Retention time: 15.415 min. ¹1H NMR (500 MHz, DMSO) δ 12.00 (s, 1H), 10.25 (s, 1H), 8.83 (s, 1H), 8.34 (t, J=7.6 Hz, 1H), 7.58 (d, J=8.7 Hz, 1H), 7.13 (d, J=8.9 Hz, 2H), 6.93 (d, J=9.0 Hz, 2H), 6.76 (s, 1H), 5.84 (s, 1H), 4.98 (s, 1H), 4.65 (dd, J=105.3, 13.7 Hz, 2H), 4.05-3.94 (m, 1H), 3.74-3.62 (m, 4H), 3.17 (t, J=9.8 Hz, 1H), 3.10-3.02 (m, 1H), 2.98-2.90 (m, 1H), 2.72-2.62 (m, 4H), 2.56 (m, J=11.8 Hz, 1H), 2.48-2.44 (m, 1H), 2.37-2.25 (m, 3H), 2.20 (dt, J=21.3, 10.6 Hz, 1H), 2.06-1.98 (m, 1H), 1.94-1.87 (m, 1H), 1.84-1.64 (m, 6H), 1.59 (s, 1H), 1.21 (s, 11H). LC-MS (ESI): m/z [M+H]⁺=856.58.

Chiral analytical method: Column: CHIRALPAK IE 4.6*250 mm 5 μm; Mobile phase: A for MtBE (0.1%2M NH3 MeOH)(%) and B for MeOH:DCM=50:50 (v/v); Gradient: Mobile Phase A: Mobile Phase B=20:80 (v/v); HPLC Equipment: Agilent 1260 Infinity II (ARD_LC-808); Column temperature: 25° C.

Chiral Prep-HPLC Condition: CHIRALPAK IE 250*20.0 mml, 5 μm; Mobile phase: A for MtBE (0.1%2M NH3 MeOH)(%) and B for MeOH:DCM=50:50; Gradient: Mobile Phase A: Mobile Phase B=20:80 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 34: (1S,3R)-3-(3-((2-((S)-1-((1-(4-(2,4-dioxotetrahydropyrimidin-1(2H)-yl)phenyl)azetidin-3-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl tert-butylcarbamate

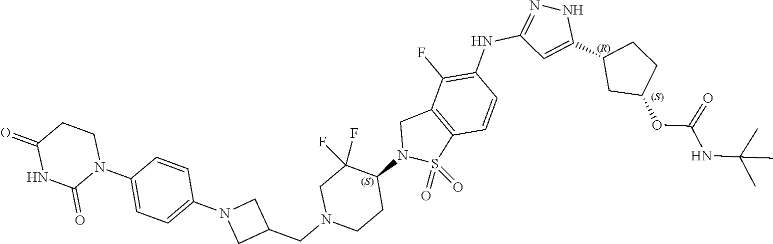

The titled compound was synthesized in the procedures similar to Example 19 in a racemic form, which was further separated by Chiral Prep-HPLC to give:

Enantiomer 1 (example 34, 100% ee); Retention time: 8.644 min. ¹H NMR (500 MHz, DMSO) δ 12.00 (s, 1H), 10.23 (s, 1H), 8.83 (s, 1H), 8.33 (t, J=8.0 Hz, 1H), 7.58 (d, J=8.7 Hz, 1H), 7.10 (d, J=8.7 Hz, 2H), 6.76 (s, 1H), 6.42 (d, J=8.7 Hz, 2H), 5.84 (s, 1H), 4.98 (s, 1H), 4.64 (dd, J=106.1, 13.7 Hz, 2H), 4.06-3.96 (m, 1H), 3.93 (t, J=7.3 Hz, 2H), 3.67 (t, J=6.7 Hz, 2H), 3.46 (t, J=6.4 Hz, 2H), 3.17 (t, J=9.8 Hz, 1H), 3.11-3.02 (m, 1H), 2.98-2.90 (m, 2H), 2.72 (d, J=7.2 Hz, 2H), 2.67 (t, J=6.7 Hz, 2H), 2.64-2.53 (m, 2H), 2.47-2.44 (m, 1H), 2.42-2.36 (m, 1H), 2.25-2.13 (m, 1H), 2.07-1.98 (m, 1H), 1.96-1.85 (m, 1H), 1.80 (d, J=12.1 Hz, 1H), 1.76-1.65 (m, 2H), 1.58 (s, 1H), 1.21 (s, 9H). LC-MS (ESI): m/z [M+H]⁺=828.71.

Chiral analytical method: Column: CHIRALPAK IE 4.6*250 mm 5 μm; Mobile phase: A for MtBE (0.1%2M NH3 MeOH)(%) and B for MeOH:DCM=50:50 (v/v); Gradient: Mobile Phase A: Mobile Phase B=20:80 (v/v); HPLC Equipment: Agilent 1260 Infinity II (ARD_LC-808); Column temperature: 25° C.

Chiral Prep-HPLC Condition: CHIRALPAK IE 250*20.0 mml, 5 μm; Mobile phase: A for MtBE (0.1%2M NH3 MeOH)(%) and B for MeOH:DCM=50:50; Gradient: Mobile Phase A: Mobile Phase B=20:80 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 35: (1S,3R)-3-(3-((2-((S)-1-((1-(4-(2,4-dioxotetrahydropyrimidin-1(2H)-yl)phenyl)piperidin-4-yl)methyl)-2,2-dimethylpiperidin-4-yl)-4-fluoro-1,1-dioxido-2,3-dihydrobenzo[d]isothiazol-5-yl)amino)-1H-pyrazol-5-yl)cyclopentyl tert-butylcarbamate

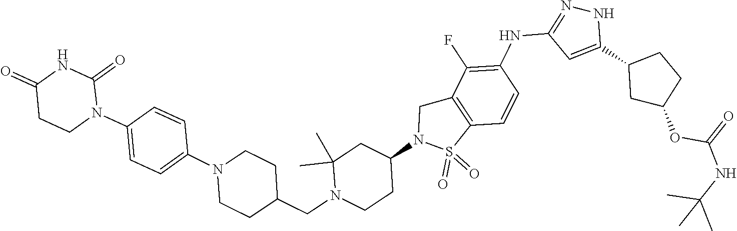 [SMILES: CC(C)(C)NC(=O)O[C@H]1CC[C@@H](c2cc(Nc3ccc4c(c3F)CN([C@H]3CCN(CC5CCN(c6ccc(N7CCC(=O)NC7=O)cc6)CC5)C(C)(C)C3)S4(=O)=O)n[nH]2)C1]

### Step 1: tert-butyl 4-(5-bromo-4-fluoro-1,1-dioxidobenzo[d]isothiazol-2(3H)-yl)-2,2-dimethylpiperidine-1-carboxylate

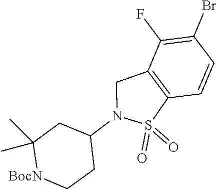 [SMILES: CC(C)(C)OC(=O)N1CCC(N2Cc3c(ccc(Br)c3F)S2(=O)=O)CC1(C)C]

To a solution of 5-bromo-4-fluoro-2,3-dihydrobenzo[d]isothiazole 1,1-dioxide (2 g, 7.55 mmol) and tert-butyl 4-hydroxy-2,2-dimethylpiperidine-1-carboxylate (3.24 g, 15.1 mmol) in toluene (30 mL) was added 2-(tributylphosphoranylidene)acetonitrile (5.45 g, 22.65 mmol). The reaction mixture was stirred under nitrogen atmosphere at 100° C. for 16 h. The resulting mixture was concentrated under reduce pressure, The residue was purified by silica gel column chromatography, eluting with PE/EA (2:1) to afford the product (1.5 g, 42.8% yield). LC-MS (ESI): m/z [M+H]⁺=477.2.

### Step 2: (1S,3R)-3-(3-((2-((S)-1-((1-(4-(2,4-dioxotetrahydropyrimidin-1(2H)-yl)phenyl)piperidin-4-yl)methyl)-2,2-dimethylpiperidin-4-yl)-4-fluoro-1,1-dioxido-2,3-dihydrobenzo[d]isothiazol-5-yl)amino)-1H-pyrazol-5-yl)cyclopentyl tert-butylcarbamate

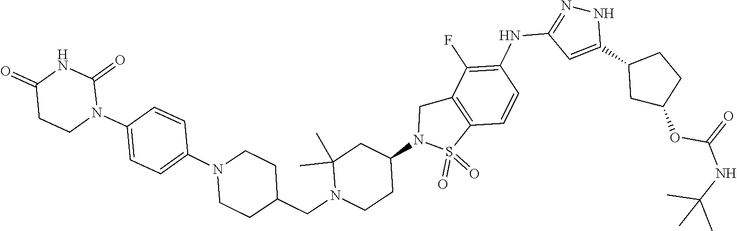 [SMILES: CC(C)(C)NC(=O)O[C@H]1CC[C@@H](c2cc(Nc3ccc4c(c3F)CN([C@H]3CCN(CC5CCN(c6ccc(N7CCC(=O)NC7=O)cc6)CC5)C(C)(C)C3)S4(=O)=O)n[nH]2)C1]

The titled compound was synthesized in the procedures similar to Example 19 in a racemic form, which was further separated by Chiral Prep-HPLC to give:

Enantiomer 1 (Example 35, 100% ee); Retention time: 13.521 min. ¹H NMR (500 MHz, DMSO) δ 11.98 (s, 1H), 10.25 (s, 1H), 8.77 (s, 1H), 8.30 (d, J=7.1 Hz, 1H), 7.54 (d, J=8.6 Hz, 1H), 7.13 (d, J=8.9 Hz, 2H), 6.93 (d, J=9.0 Hz, 2H), 6.76 (s, 1H), 5.83 (s, 1H), 5.06-4.96 (m, 1H), 4.54 (s, 2H), 3.69 (t, J=6.7 Hz, 4H), 3.22 (dd, J=12.3, 3.7 Hz, 1H), 3.12-3.00 (m, 1H), 2.95 (d, J=9.4 Hz, 1H), 2.72-2.62 (m, 4H), 2.47-2.41 (m, 1H), 2.24-2.07 (m, 3H), 2.05-1.87 (m, 3H), 1.84-1.47 (m, 9H), 1.26-1.17 (m, 11H), 1.15-1.06 (m, 3H), 0.94-0.93 (m, 3H). LC-MS (ESI): m/z [M+H]⁺=848.4;

Chiral analytical method: Column: ICHIRALPAK IB N-5 4.6*150 mm 5 μm; Mobile phase: A for MtBE (0.1%2M NH3 MeOH) (%) and B for MeOH:DCM=50:50(%); Gradient: Mobile Phase A: Mobile Phase B=80:20 (v/v); HPLC Equipment: HPLC-Agilent, Back pressure: 100 bar; Column temperature: 35° C.

Chiral Prep-HPLC Condition: CHIRALPAK B N-5 250*20.0 mml, 5 μm; Mobile phase: A for MtBE (0.1%2M NH3 MeOH) (%) and B for MeOH:DCM=50:50; Gradient: Mobile Phase A: Mobile Phase B=80:20 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 36: (1S,3R)-3-(3-((2-(1-((1-(1-(2,6-dioxopiperidin-3-yl)-3-methyl-2-oxo-2,3-dihydro-1H-benzo[d]imidazol-5-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

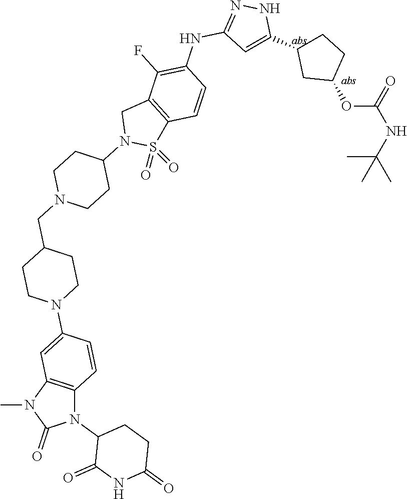 [SMILES: Cn1c(=O)n(C2CCC(=O)NC2=O)c2ccc(N3CCC(CN4CCC(N5Cc6c(ccc(Nc7cc([C@@H]8CC[C@H](OC(=O)NC(C)(C)C)C8)[nH]n7)c6F)S5(=O)=O)CC4)CC3)cc21]

The titled compound was synthesized in the procedures similar to Example 1. ¹H NMR (500 MHz, DMSO-d₆) δ 11.99 (s, 1H), 11.06 (s, 1H), 8.77 (s, 1H), 8.29 (s, 1H), 7.51 (d, J=10 Hz, 1H), 6.95-6.89 (m, 1H), 6.83-6.80 (m, 1H), 6.79-6.73 (m, 1H), 6.66-6.60 (m, 1H), 5.83 (s, 1H), 5.32-5.25 (m, 1H), 5.01-4.94 (m, 1H), 4.52 (s, 2H), 3.62-3.53 (m, 2H), 3.28-3.39 (m, 1H), 3.30 (s, 3H), 3.10-3.01 (m, 1H), 2.93-2.84 (m, 3H), 2.71-2.51 (m, 4H), 2.46-2.43 (m, 1H), 2.21-2.15 (m, 2H), 2.08-1.95 (m, 5H), 1.95-1.84 (m, 4H), 1.83-1.77 (m, 2H), 1.74-1.67 (m, 2H), 1.66-1.54 (m, 2H), 1.30-1.17 (m, 12H). LC-MS (ESI): m/z [M+H]⁺=889.

### Example 37: (1S,3R)-3-(3-((2-(1-((1-(1-(2,6-dioxopiperidin-3-yl)-4-fluoro-3-methyl-2-oxo-2,3-dihydro-1H-benzo[d]imidazol-5-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

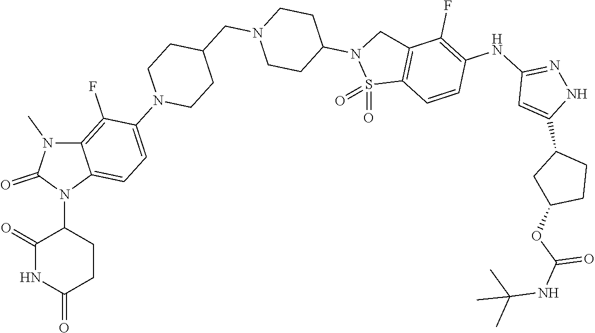 [SMILES: Cn1c(=O)n(C2CCC(=O)NC2=O)c2ccc(N3CCC(CN4CCC(N5Cc6c(ccc(Nc7cc([C@@H]8CC[C@H](OC(=O)NC(C)(C)C)C8)[nH]n7)c6F)S5(=O)=O)CC4)CC3)c(F)c21]

### Step 1: 3-bromo-2-fluoro-N-methyl-6-nitroaniline

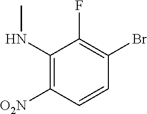

To a solution of 1-bromo-2,3-difluoro-4-nitrobenzene (16 g, 67.2 mmol) in DMF (160 mL) were added K₂CO₃ (18.5 g, 134.4 mmol) and methanamine (35.2 mL, 80.6 mmol) stirred for 12 h at room temperature. The resulting solution was extracted with 3×200 mL of EtOAc. The combined organic phases were concentrated under reduced pressure. The residue was purified by combi-flash (EtOAc/PE=10%) to give the product (16 g, 95.8%) as yellow solid. MS (ESI) m/e [M+1]⁺=249/251.

### Step 2: 5-bromo-6-fluoro-N1-methylbenzene-1,2-diamine

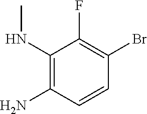

To a solution of 3-bromo-2-fluoro-N-methyl-6-nitroaniline (16 g, 64.3 mmol) in MeOH:H₂O=4:1 (250 mL) were added Fe (10.8 g, 192.9 mmol) and NH₄Cl (17 g, 321.5 mmol). The resulting solution was stirred for 3 h at 80° C. The resulting solution was extracted with 3×160 mL of EtOAc. The resulting mixture was concentrated under vacuum. The residue was purified by combi-flash (EtOAc/PE=16%) to give the product (13.4 g, 95%) as yellow solid. MS (ESI) m/e [M+1]⁺=219/221.

### Step 3: 6-bromo-7-fluoro-1-methyl-1,3-dihydro-2H-benzo[d]imidazol-2-one

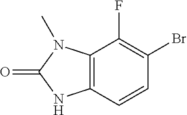

To a solution of 5-bromo-6-fluoro-N1-methylbenzene-1,2-diamine (13.4 g, 61.2 mmol) in MeCN (100 mL) was added CDI (14.9 g, 91.8 mmol) under oxygen atmosphere. The resulting solution was stirred for 3 h at 85° C. under nitrogen atmosphere. The resulting mixture was concentrated under vacuum. The residue was purified by combi-flash (EtOAc/PE=40%) to give the product (12.6 g, 84%) as red solid. MS (ESI) m/e [M+1]⁺=245/247.

### Step 4: 1-(2,6-bis(benzyloxy)pyridin-3-yl)-5-bromo-4-fluoro-3-methyl-1,3-dihydro-2H-benzo[d]imidazol-2-one

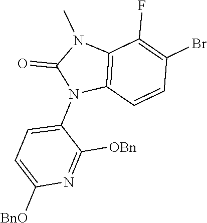 [SMILES: Cn1c(=O)n(-c2ccc(OCc3ccccc3)nc2OCc2ccccc2)c2ccc(Br)c(F)c21]

To a solution of 6-bromo-7-fluoro-1-methyl-1,3-dihydro-2H-benzo[d]imidazol-2-one (12.5 g, 51 mmol) in dioxane (300 mL) were added 2,6-bis(benzyloxy)-3-(4,4,5,5-tetramethyl-1,3,2-dioxaborolan-2-yl) pyridine (38.4 g, 76.5 mmol), Cu(OAc)₂ (9.3 g, 51 mmol), Pyridine (12.1 g, 153 mmol) and 4A MS (12 g). The resulting solution was stirred for 20 h at 80° C. under oxygen atmosphere. After filtration, the filtrate was concentrated under vacuum. The residue was purified by combi-flash (EtOAc/PE=6%) to give the product (18.8 g, 69.1%) as red solid. MS (ESI) m/e [M+1]⁺=534/536.

### Step 5: 1-(2,6-bis(benzyloxy)pyridin-3-yl)-5-(4-(dimethoxymethyl) piperidin-1-yl)-4-fluoro-3-methyl-1,3-dihydro-2H-benzo[d]imidazol-2-one

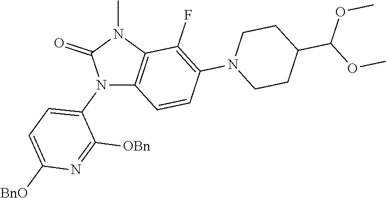 [SMILES: COC(OC)C1CCN(c2ccc3c(c2F)n(C)c(=O)n3-c2ccc(OCc3ccccc3)nc2OCc2ccccc2)CC1]

To a solution of 1-(2,6-bis(benzyloxy)pyridin-3-yl)-5-bromo-4-fluoro-3-methyl-1,3-dihydro-2H-benzo[d]imidazol-2-one (4 g, 7.5 mmol) in dioxane (80 mL) were added 4-(dimethoxymethyl)piperidine (1.4 g, 9 mmol), Pd-PEPPSI-IPentCl 2-methylpyridine (o-picoline) (315 mg, 0.4 mmol) and Cs₂CO₃ (7.3 g, 22.5 mmol). The resulting solution was stirred for 3 h at 100° C. under nitrogen atmosphere. The resulting mixture was concentrated under vacuum. The residue was purified by combi-flash (EtOAc/PE=43%) to give the product (4.2 g, 91.3%) as yellow solid. MS (ESI) m/e [M+1]⁺=613.

### Step 6: 3-(5-(4-(dimethoxymethyl) piperidin-1-yl)-4-fluoro-3-methyl-2-oxo-2,3-dihydro-1H-benzo[d]imidazol-1-yl) piperidine-2,6-dione

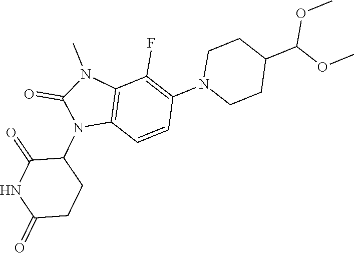 [SMILES: COC(OC)C1CCN(c2ccc3c(c2F)n(C)c(=O)n3C2CCC(=O)NC2=O)CC1]

To a solution of 1-(2,6-bis(benzyloxy)pyridin-3-yl)-5-(4-(dimethoxymethyl) piperidin-1-yl)-4-fluoro-3-methyl-1,3-dihydro-2H-benzo[d]imidazol-2-one (2.1 g, 3.4 mmol) in THF (40 mL) was added Pd/C (2.1 g, 10% wt). The resulting solution was stirred for 12 h at 50° C. under H₂ atmosphere. After filtration, the filtrate was concentrated under vacuum. The residue was the product (1 g, 66.7%) as grey solid. MS (ESI) m/e [M+1]⁺=435.

### Step 7: 1-(1-(2,6-dioxopiperidin-3-yl)-4-fluoro-3-methyl-2-oxo-2,3-dihydro-1H-benzo[d]imidazol-5-yl)piperidine-4-carbaldehyde

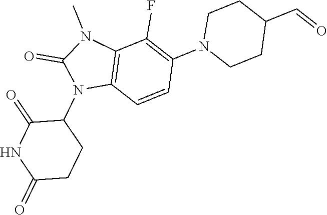 [SMILES: Cn1c(=O)n(C2CCC(=O)NC2=O)c2ccc(N3CCC(C=O)CC3)c(F)c21]

3-(5-(4-(dimethoxymethyl) piperidin-1-yl)-4-fluoro-3-methyl-2-oxo-2,3-dihydro-1H-benzo[d]imidazol-1-yl) piperidine-2,6-dione (130 mg, 0.3 mmol) was dissolved in formic acid (5 mL), stirred for 2 hours at room temperature. The resulting mixture was concentrated under reduce pressure, and dried over in vacuum to afford the product (110 mg, 95%). LC-MS (ESI): m/z [M+H]⁺=389.4.

### Step 8: (1S,3R)-3-(3-((2-(1-((1-(1-(2,6-dioxopiperidin-3-yl)-4-fluoro-3-methyl-2-oxo-2,3-dihydro-1H-benzo[d]imidazol-5-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

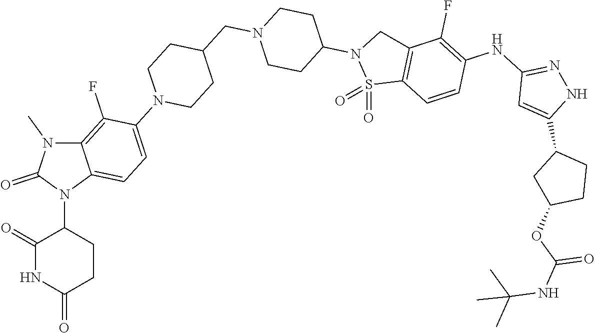 [SMILES: Cn1c(=O)n(C2CCC(=O)NC2=O)c2ccc(N3CCC(CN4CCC(N5Cc6c(ccc(Nc7cc([C@@H]8CC[C@H](OC(=O)NC(C)(C)C)C8)[nH]n7)c6F)S5(=O)=O)CC4)CC3)c(F)c21]

The titled compound was synthesized in the procedures similar to Example 1. ¹H NMR (500 MHz, DMSO) δ 11.99 (s, 1H), 11.10 (s, 1H), 8.78 (s, 1H), 8.30 (s, 1H), 7.52 (d, J=8.4 Hz, 1H), 5.83 (s, 1H), 5.33 (dd, J=12.7, 5.1 Hz, 1H), 5.06-4.96 (m, 1H), 4.53 (s, 2H), 3.47 (s, 4H), 3.24 (d, J=10.2 Hz, 2H), 3.11-3.00 (m, 2H), 2.98-2.79 (m, 3H), 2.66 (m, 6H), 2.46-2.42 (m, 1H), 2.23-1.99 (m, 2H), 1.99-1.85 (m, 4H), 1.85-1.77 (m, 3H), 1.82 (m, 3H), 1.75-1.65 (m, 3H), 1.62-1.56 (m, 1H), 1.33 (m, 2H), 1.21 (s, 9H). LC-MS (ESI): m/z [M+H]⁺=907.4;

### Example 38: (1S,3R)-3-(3-((2-(1-((1-(3-cyclopropyl-1-(2,6-dioxopiperidin-3-yl)-4-fluoro-2-oxo-2,3-dihydro-1H-benzo[d]imidazol-5-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

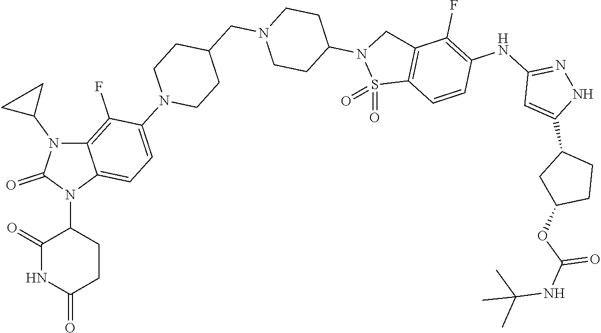 [SMILES: CC(C)(C)NC(=O)O[C@H]1CC[C@@H](c2cc(Nc3ccc4c(c3F)CN(C3CCN(CC5CCN(c6ccc7c(c6F)n(C6CC6)c(=O)n7C6CCC(=O)NC6=O)CC5)CC3)S4(=O)=O)n[nH]2)C1]

### Step 1: 2,6-bis(benzyloxy)-N-(4-bromo-2-fluoro-6-nitrophenyl)pyridin-3-amine

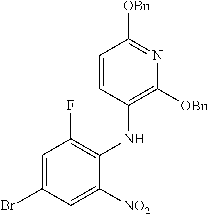 [SMILES: O=[N+]([O-])c1cc(Br)cc(F)c1Nc1ccc(OCc2ccccc2)nc1OCc1ccccc1]

To a solution of 2,6-bis(benzyloxy)pyridin-3-amine (5.38 g, 17.58 mmol) in THF (100 mL) was added LiHMDS (1M) (35 mL, 35.16 mmol) at 0° C. for 30 min under N₂. The 5-bromo-1,2-difluoro-3-nitrobenzene (5 g, 21 mmol) was added in it at rt for overnight. The resulting mixture was quenched with NH₄Cl solution and extracted with EA. The organic layers were washed with brine and concentrated under vacuum. The residue was purified by combi-flash (PE/EA=100:0 to 94:6) to give the product (5 g, 45.4%) as black oil.

### Step 2: N¹-(2,6-bis(benzyloxy)pyridin-3-yl)-4-bromo-6-fluorobenzene-1,2-diamine

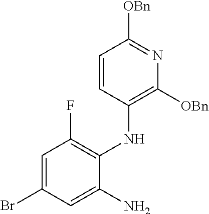 [SMILES: Nc1cc(Br)cc(F)c1Nc1ccc(OCc2ccccc2)nc1OCc1ccccc1]

To a solution of 2,6-bis(benzyloxy)-N-(4-bromo-2-fluoro-6-nitrophenyl)pyridin-3-amine (5 g, 9.56 mmol) in MeOH/H₂O=4:1 (100 mL) were added Fe (1.8 g, 28.68 mmol) and NH₄Cl (2.8 g, 47.8 mmol) at 70° C. for 1 h. The resulting solution was filtered, the filter cake was washed with DCM (50 mL). The filtrate was concentrated under vacuum. The residue was purified by combi-flash (PE/EA=100:0 to 91:9) to give the product (4.6 g, 97.5%) as red solid.

### Step 3:1-(2,6-bis(benzyloxy)pyridin-3-yl)-5-bromo-7-fluoro-1,3-dihydro-2H-benzo[d]imidazol-2-one

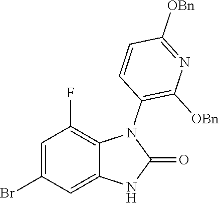 [SMILES: O=c1[nH]c2cc(Br)cc(F)c2n1-c1ccc(OCc2ccccc2)nc1OCc1ccccc1]

To a solution of N¹-(2,6-bis(benzyloxy)pyridin-3-yl)-4-bromo-6-fluorobenzene-1,2-diamine (4.3 g, 8.72 mmol) in CH₃CN (100 mL) was added CDI (2.11 g, 13.08 mmol) at 85° C. for 12 h. The reaction was concentrated under vacuum. The residue was purified by combi-flash (PE/EA=100:0 to 60:40) to give the product (3.3 g, 73.3%) as light brown solid.

### Step 4: 1-(2,6-bis(benzyloxy)pyridin-3-yl)-5-bromo-3-cyclopropyl-4-fluoro-1,3-dihydro-2H-benzo[d]imidazol-2-one

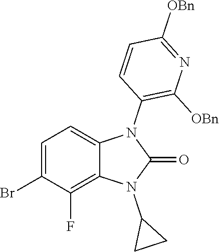 [SMILES: O=c1n(-c2ccc(OCc3ccccc3)nc2OCc2ccccc2)c2ccc(Br)c(F)c2n1C1CC1]

To a solution of 1-(2,6-bis(benzyloxy)pyridin-3-yl)-5-bromo-4-fluoro-1,3-dihydro-2H-benzo[d]imidazol-2-one (1 g, 1.92 mmol) and potassium cyclopropyltrifluoroborate (570 mg, 3.84 mmol) in Toluene (20 mL), H2O (2 mL) was added 1,10-Phenthroline (173 mg, 0.96 mmol), Cu(OAc)2 (173 mg, 0.96 mmol) and K₂CO₃ (795 mg, 5.76 mmol), the reaction mixture was stirred for 16 hours at 100° C. under nitrogen. The resulting mixture was cooled to room temperature, filtered, the filtrate was concentrated under reduce pressure. The residue was purified by silica gel column chromatography, eluting with PE/EA (1:2) to afford the product (900 mg, 90% yield). LC-MS (ESI): m/z [M+H]⁺=560.3.

### Step 5: 1-(2,6-bis(benzyloxy)pyridin-3-yl)-5-bromo-3-cyclopropyl-4-fluoro-1,3-dihydro-2H-benzo[d]imidazol-2-one

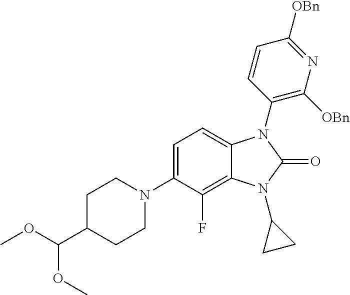 [SMILES: COC(OC)C1CCN(c2ccc3c(c2F)n(C2CC2)c(=O)n3-c2ccc(OCc3ccccc3)nc2OCc2ccccc2)CC1]

To a solution of 1-(2,6-bis(benzyloxy)pyridin-3-yl)-5-bromo-3-cyclopropyl-4-fluoro-1,3-dihydro-2H-benzo[d]imidazol-2-one (300 mg, 0.71 mmol) and 4-(dimethoxymethyl)piperidine (227 mg, 1.42 mmol) in dioxane (20 mL) was added Pd-PEPPSI-IPentCl 2-methylpyridine (30 mg, 0.035 mmol) and Cs₂CO₃ (694 mg, 2.13 mmol), the reaction mixture was stirred for 3 hours at 100° C. under nitrogen. The resulting mixture was cooled to room temperature, filtered, the filtrate was concentrated under reduce pressure. The residue was purified by silica gel column chromatography, eluting with EA (100%) to afford the product (300 mg, 87.6% yield). LC-MS (ESI): m/z [M+H]⁺=639.2.

### Step 6: 3-(3-cyclopropyl-5-(4-(dimethoxymethyl)piperidin-1-yl)-4-fluoro-2-oxo-2,3-dihydro-1H-benzo[d]imidazol-1-yl)piperidine-2,6-dione

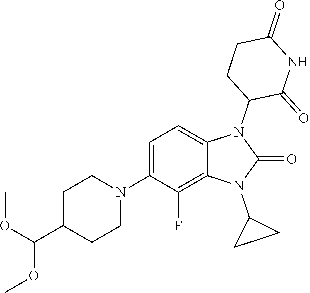 [SMILES: COC(OC)C1CCN(c2ccc3c(c2F)n(C2CC2)c(=O)n3C2CCC(=O)NC2=O)CC1]

To a solution of 1-(2,6-bis(benzyloxy)pyridin-3-yl)-5-bromo-3-cyclopropyl-4-fluoro-1,3-dihydro-2H-benzo[d]imidazol-2-one (300 mg, 1.64 mmol) in THF (30 mL) was added Pd/C (150 mg, 10% wt). The resulting solution was stirred for 5 h at 50° C. under H₂ atmosphere. After filtration, the filtrate was concentrated under vacuum. The residue was the product (200 mg, 92.5%)) as green solid. LC-MS (ESI): m/z [M+H]⁺=461.3.

### Step 7: 1-(3-cyclopropyl-1-(2,6-dioxopiperidin-3-yl)-4-fluoro-2-oxo-2,3-dihydro-1H-benzo[d]imidazol-5-yl)piperidine-4-carbaldehyde

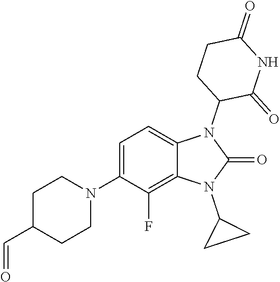 [SMILES: O=CC1CCN(c2ccc3c(c2F)n(C2CC2)c(=O)n3C2CCC(=O)NC2=O)CC1]

3-(3-cyclopropyl-5-(4-(dimethoxymethyl)piperidin-1-yl)-4-fluoro-2-oxo-2,3-dihydro-1H-benzo[d]imidazol-1-yl)piperidine-2,6-dione (200 mg, 0.3 mmol) was dissolved in formic acid (5 mL), stirred for 2 hours at room temperature. The resulting mixture was concentrated under reduce pressure, dried over in vacuum, to afford the product (160 mg, 88.8%). LC-MS (ESI): m/z [M+H]⁺=415.4.

### Step 8: (1S,3R)-3-(3-((2-(1-((1-(3-cyclopropyl-1-(2,6-dioxopiperidin-3-yl)-4-fluoro-2-oxo-2,3-dihydro-1H-benzo[d]imidazol-5-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

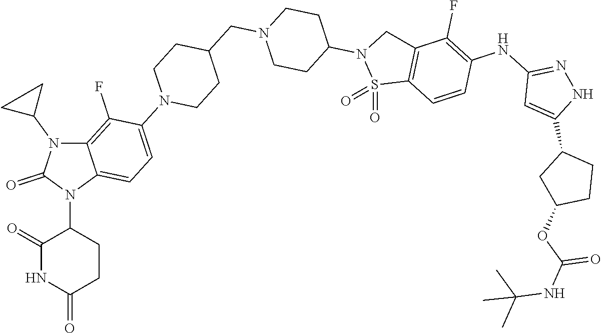 [SMILES: CC(C)(C)NC(=O)O[C@H]1CC[C@@H](c2cc(Nc3ccc4c(c3F)CN(C3CCN(CC5CCN(c6ccc7c(c6F)n(C6CC6)c(=O)n7C6CCC(=O)NC6=O)CC5)CC3)S4(=O)=O)n[nH]2)C1]

The titled compound was synthesized in the procedures similar to Example 37. ¹H NMR (500 MHz, DMSO) δ 11.99 (s, 1H), 11.08 (s, 1H), 8.76 (s, 1H), 8.29 (s, 1H), 7.51 (d, J=8.7 Hz, 1H), 6.94 (d, J=7.3 Hz, 1H), 6.80 (d, J=8.6 Hz, 1H), 6.75 (t, J=8.0 Hz, 1H), 5.83 (s, 1H), 5.28 (dd, J=12.7, 5.3 Hz, 1H), 5.06-4.96 (m, 1H), 4.52 (s, 2H), 3.60-3.54 (m, 1H), 3.49-3.41 (m, 1H), 3.22 (d, J=11.0 Hz, 2H), 3.10-3.02 (m, 2H), 2.96-2.82 (m, 3H), 2.70-2.60 (m, 4H), 2.45-2.42 (m, 1H), 2.21 (d, J=6.7 Hz, 2H), 2.10-1.97 (m, 4H), 1.96-1.78 (m, 8H), 1.76-1.58 (m, 4H), 1.37-1.23 (m, 2H), 1.06-0.94 (m, 13H). LC-MS (ESI): m/z [M+H]⁺=933.4;

### Example 39: (1S,3R)-3-(3-((2-(1′-(1-(2,6-dioxopiperidin-3-yl)-3-ethyl-4-fluoro-2-oxo-2,3-dihydro-1H-benzo[d]imidazol-5-yl)-[1,4′-bipiperidin]-4-yl)-4-fluoro-1,1-dioxido-2,3-dihydrobenzo[d]isothiazol-5-yl)amino)-1H-pyrazol-5-yl)cyclopentyl tert-butylcarbamate

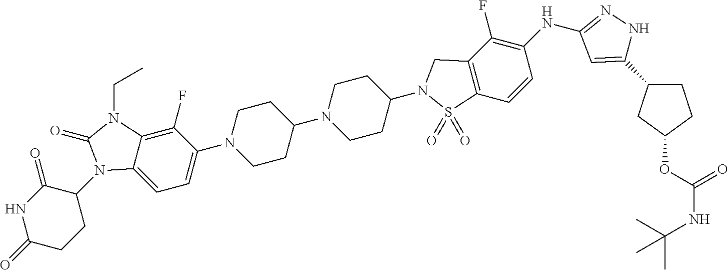 [SMILES: CCn1c(=O)n(C2CCC(=O)NC2=O)c2ccc(N3CCC(N4CCC(N5Cc6c(ccc(Nc7cc([C@@H]8CC[C@H](OC(=O)NC(C)(C)C)C8)[nH]n7)c6F)S5(=O)=O)CC4)CC3)c(F)c21]

### Step 1: 1-(2,6-bis(benzyloxy)pyridin-3-yl)-5-bromo-3-ethyl-4-fluoro-1,3-dihydro-2H-benzo[d]imidazol-2-one

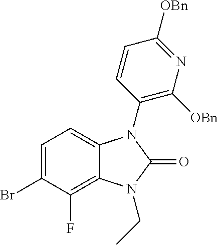 [SMILES: CCn1c(=O)n(-c2ccc(OCc3ccccc3)nc2OCc2ccccc2)c2ccc(Br)c(F)c21]

To a solution of 1-(2,6-bis(benzyloxy)pyridin-3-yl)-5-bromo-4-fluoro-1,3-dihydro-2H-benzo[d]imidazol-2-one (1 g, 1.93 mmol) in DMF (60 mL) were added K₂CO₃ (533 mg, 3.86 mmol) and iodoethane (331 mg, 2.12 mmol). The resulting solution was stirred for 3 h at room temperature. The resulting solution was extracted with 3×60 mL of EA. The resulting mixture was concentrated under vacuum. The residue was purified by combi-flash (PE/EA=3:1) to give the product (1 g, 92.9%) as red solid. LC-MS (ESI): m/z [M+H]⁺=548.3

### Step 2: 1-(2,6-bis(benzyloxy)pyridin-3-yl)-3-ethyl-4-fluoro-5-(1,4-dioxa-8-azaspiro[4.5]decan-8-yl)-1,3-dihydro-2H-benzo[d]imidazol-2-one

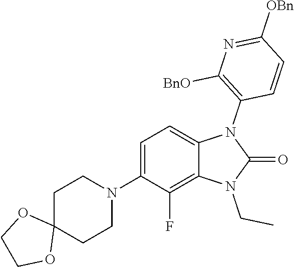 [SMILES: CCn1c(=O)n(-c2ccc(OCc3ccccc3)nc2OCc2ccccc2)c2ccc(N3CCC4(CC3)OCCO4)c(F)c21]

To a solution of 1-(2,6-bis(benzyloxy)pyridin-3-yl)-5-bromo-3-ethyl-4-fluoro-1,3-dihydro-2H-benzo[d]imidazol-2-one (1 g, 1.82 mmol) in dioxane (50 mL) were added 1,4-dioxa-8-azaspiro[4.5]decane (522 mg, 3.65 mmol), Pd-PEPPSI-IPentCl 2-methylpyridine (o-picoline) (151.2 mg, 0.18 mmol) and Cs₂CO₃ (1.77 g, 5.46 mmol). The resulting solution was stirred for 12 h at 100° C. under nitrogen atmosphere. The resulting mixture was concentrated under vacuum. The residue was purified by combi-flash (PE/EA=1:1) to give the product (1 g, 90%) as yellow solid. LC-MS (ESI): m/z [M+H]⁺=611.3.

### Step 3: 3-(3-ethyl-4-fluoro-2-oxo-5-(1,4-dioxa-8-azaspiro[4.5]decan-8-yl)-2,3-dihydro-1H-benzo[d]imidazol-1-yl)piperidine-2,6-dione

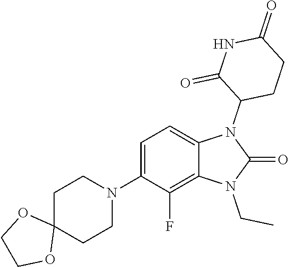 [SMILES: CCn1c(=O)n(C2CCC(=O)NC2=O)c2ccc(N3CCC4(CC3)OCCO4)c(F)c21]

To a solution of 1-(2,6-bis(benzyloxy)pyridin-3-yl)-3-ethyl-4-fluoro-5-(1,4-dioxa-8-azaspiro[4.5]decan-8-yl)-1,3-dihydro-2H-benzo[d]imidazol-2-one (1 g, 1.64 mmol) in THF (30 mL) was added Pd/C (500 mg, 10% wt). The resulting solution was stirred for 12 h at 50° C. under H₂ atmosphere. After filtration, the filtrate was concentrated under vacuum. The residue was the product ((650 mg, 91.9%)) as green solid. LC-MS (ESI): m/z [M+H]⁺=433.3.

### Step 4: 3-(3-ethyl-4-fluoro-2-oxo-5-(4-oxopiperidin-1-yl)-2,3-dihydro-1H-benzo[d]imidazol-1-yl)piperidine-2,6-dione

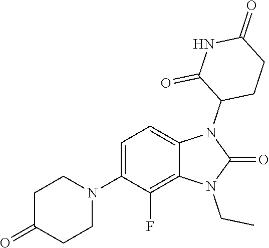 [SMILES: CCn1c(=O)n(C2CCC(=O)NC2=O)c2ccc(N3CCC(=O)CC3)c(F)c21]

The solution of 3-(3-ethyl-4-fluoro-2-oxo-5-(1,4-dioxa-8-azaspiro[4.5]decan-8-yl)-2,3-dihydro-1H-benzo[d]imidazol-1-yl)piperidine-2,6-dione (650 mg, 1.5 mmol) in H₂SO₄ (3 mL) was stirred for 3 h at room temperature. The residue was neutralized to pH 7 with Na₂CO₃ aqueous solution. The resulting solution was extracted with 3×20 mL of EtOAc. The resulting mixture was concentrated under vacuum. The residue was purified by trituration (EtOAc) to give the product (500 mg, 85.7%) as yellow solid. LC-MS (ESI): m/z [M+H]⁺=389.3.

### Step 5: (1S,3R)-3-(3-((2-(1′-(1-(2,6-dioxopiperidin-3-yl)-3-ethyl-4-fluoro-2-oxo-2,3-dihydro-1H-benzo[d]imidazol-5-yl)-[1,4′-bipiperidin]-4-yl)-4-fluoro-1,1-dioxido-2,3-dihydrobenzo[d]isothiazol-5-yl)amino)-1H-pyrazol-5-yl)cyclopentyl tert-butylcarbamate

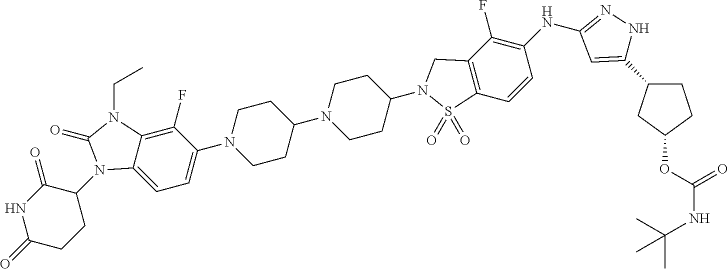 [SMILES: CCn1c(=O)n(C2CCC(=O)NC2=O)c2ccc(N3CCC(N4CCC(N5Cc6c(ccc(Nc7cc([C@@H]8CC[C@H](OC(=O)NC(C)(C)C)C8)[nH]n7)c6F)S5(=O)=O)CC4)CC3)c(F)c21]

The titled compound was synthesized in the procedures similar to Example 37. ¹H NMR (500 MHz, DMSO) δ 12.01 (s, 1H), 11.11 (s, 1H), 9.16 (s, 1H), 8.81 (s, 1H), 8.31 (s, 1H), 7.56 (t, J=10.4 Hz, 1H), 6.89 (d, J=8.5 Hz, 1H), 6.83-6.68 (m, 2H), 5.84 (s, 1H), 5.41-5.28 (m, 1H), 5.06-4.96 (m, 1H), 4.57 (s, 2H), 3.98-3.90 (m, 2H), 3.89-3.81 (m, 1H), 3.68-3.59 (m, 2H), 3.43-3.38 (m, 1H), 3.39 (s, 3H), 3.28-3.20 (m, 2H), 3.10-3.01 (m, 1H), 2.94-2.84 (m, 1H), 2.78-2.68 (m, 3H), 2.45-2.38 (m, 2H), 2.29-2.23 (m, 2H), 2.21-2.08 (m, 4H), 2.06-1.98 (m 3H), 1.96-1.81 (m, 3H), 1.75-1.64 (m, 3H), 1.62-1.52 (m, 1H), 1.28-1.24 (m, 3H), 1.21 (s, 9H). LC-MS (ESI): m/z [M+H]⁺=907.5

### Example 40: (1S,3R)-3-(3-((2-(1-((1-(1-(2,6-dioxopiperidin-3-yl)-4-fluoro-3-isopropyl-2-oxo-2,3-dihydro-1H-benzo[d]imidazol-5-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

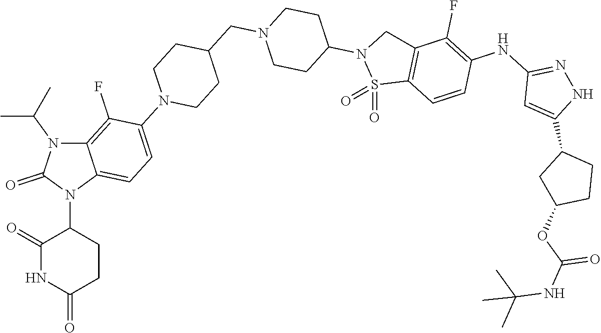 [SMILES: CC(C)n1c(=O)n(C2CCC(=O)NC2=O)c2ccc(N3CCC(CN4CCC(N5Cc6c(ccc(Nc7cc([C@@H]8CC[C@H](OC(=O)NC(C)(C)C)C8)[nH]n7)c6F)S5(=O)=O)CC4)CC3)c(F)c21]

### Step 1: 1-(1-(2,6-dioxopiperidin-3-yl)-4-fluoro-3-isopropyl-2-oxo-2,3-dihydro-1H-benzo[d]imidazol-5-yl)piperidine-4-carbaldehyde

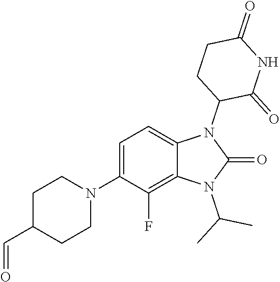 [SMILES: CC(C)n1c(=O)n(C2CCC(=O)NC2=O)c2ccc(N3CCC(C=O)CC3)c(F)c21]

The titled compound was synthesized in the procedures similar to Example 37. LC-MS (ESI): m/z [M+H]⁺=417.3.

### Step 2: (1S,3R)-3-(3-((2-(1-((1-(1-(2,6-dioxopiperidin-3-yl)-4-fluoro-3-isopropyl-2-oxo-2,3-dihydro-1H-benzo[d]imidazol-5-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

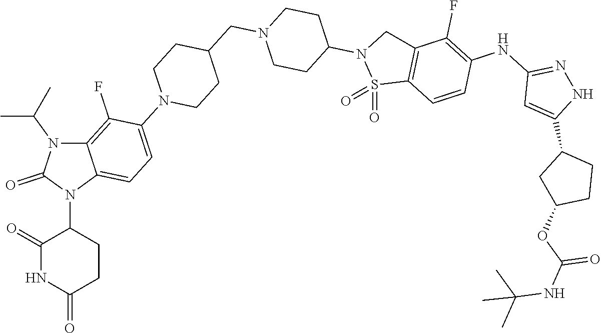 [SMILES: CC(C)n1c(=O)n(C2CCC(=O)NC2=O)c2ccc(N3CCC(CN4CCC(N5Cc6c(ccc(Nc7cc([C@@H]8CC[C@H](OC(=O)NC(C)(C)C)C8)[nH]n7)c6F)S5(=O)=O)CC4)CC3)c(F)c21]

The titled compound was synthesized in the procedures similar to Example 37. ¹H NMR (500 MHz, DMSO) δ 12.00 (s, 1H), 11.10 (s, 1H), 8.80 (s, 1H), 8.31 (s, 1H), 7.54 (d, J=7.8 Hz, 1H), 5.84 (s, 1H), 5.45-5.26 (m, 1H), 5.06-4.96 (m, 1H), 4.81-4.73 (m, 1H), 4.55 (s, 2H), 3.86-3.50 (m, 2H), 3.30-3.27 (m, 2H), 3.26-3.22 (m, 3H), 3.10-3.00 (m, 2H), 2.93-2.82 (m, 2H), 2.73-2.66 (m, 3H), 2.61-2.58 (m, 1H), 2.54-2.52 (m, 1H), 2.47-2.45 (m, 1H), 2.22-2.12 (m, 2H), 2.05-1.99 (m, 3H), 1.96-1.80 (m, 5H), 1.78-1.66 (m, 3H), 1.64-1.53 (m, 1H), 1.42 (d, J=6.7 Hz, 6H), 1.21 (s, 9H). LC-MS (ESI): m/z [M+H]⁺=935.5

### Example 41: (1S,3R)-3-(3-((2-(1-((1-(1-((S)-2,6-dioxopiperidin-3-yl)-7-fluoro-3-methyl-2-oxo-2,3-dihydro-1H-benzo[d]imidazol-5-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

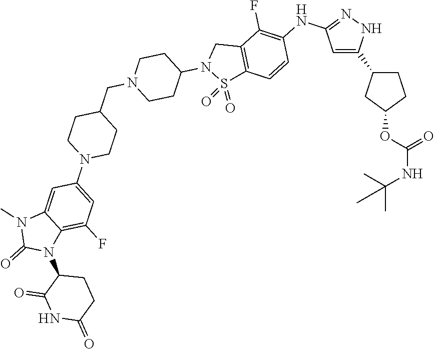 [SMILES: Cn1c(=O)n([C@H]2CCC(=O)NC2=O)c2c(F)cc(N3CCC(CN4CCC(N5Cc6c(ccc(Nc7cc([C@@H]8CC[C@H](OC(=O)NC(C)(C)C)C8)[nH]n7)c6F)S5(=O)=O)CC4)CC3)cc21]

### Step 1: 2,6-bis(benzyloxy)-N-(4-bromo-2-fluoro-6-nitrophenyl)pyridin-3-amine

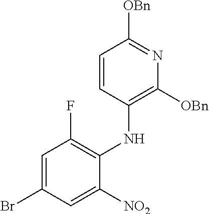 [SMILES: O=[N+]([O-])c1cc(Br)cc(F)c1Nc1ccc(OCc2ccccc2)nc1OCc1ccccc1]

To a solution of 2,6-bis(benzyloxy)pyridin-3-amine (5.38 g, 17.58 mmol) in THF (100 mL) was added LiHMDS (1M) (35 mL, 35.16 mmol) at 0° C. for 30 min under N₂. The 5-bromo-1,2-difluoro-3-nitrobenzene (5 g, 21 mmol) was added in it at rt for overnight. The resulting mixture was quenched with NH₄Cl solution and extracted with EA. The organic layers were washed with brine and concentrated under vacuum. The residue was purified by combi-flash (PE/EA=100:0 to 94:6) to give the product (5 g, 45.4%) as black oil.

### Step 2: N¹-(2,6-bis(benzyloxy)pyridin-3-yl)-4-bromo-6-fluorobenzene-1,2-diamine

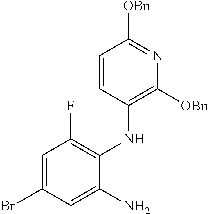 [SMILES: Nc1cc(Br)cc(F)c1Nc1ccc(OCc2ccccc2)nc1OCc1ccccc1]

To a solution of 2,6-bis(benzyloxy)-N-(4-bromo-2-fluoro-6-nitrophenyl)pyridin-3-amine (5 g, 9.56 mmol) in MeOH/H₂O=4:1 (100 mL) were added Fe (1.8 g, 28.68 mmol) and NH₄Cl (2.8 g, 47.8 mmol) at 70° C. for 1 h. The resulting solution was filtered. The filter cake was washed with DCM (50 mL). The filtrate was concentrated under vacuum. The residue was purified by combi-flash (PE/EA=100:0 to 91:9) to give the product (4.6 g, 97.5%) as red solid.

### Step 3: 1-(2,6-bis(benzyloxy)pyridin-3-yl)-5-bromo-7-fluoro-1,3-dihydro-2H-benzo[d]imidazol-2-one

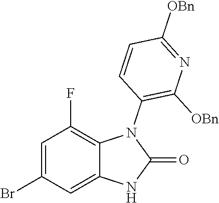 [SMILES: O=c1[nH]c2cc(Br)cc(F)c2n1-c1ccc(OCc2ccccc2)nc1OCc1ccccc1]

To a solution of N¹-(2,6-bis(benzyloxy)pyridin-3-yl)-4-bromo-6-fluorobenzene-1,2-diamine (4.3 g, 8.72 mmol) in CH₃CN (100 mL) was added CDI (2.11 g, 13.08 mmol) at 85° C. for 12 h. The reaction was concentrated under vacuum. The residue was purified by combi-flash (PE/EA=100:0 to 60:40) to give the product (3.3 g, 73.3%) as light brown solid.

### Step 4: 3-(2,6-bis(benzyloxy)pyridin-3-yl)-6-bromo-4-fluoro-1-methyl-1,3-dihydro-2H-benzo[d]imidazol-2-one

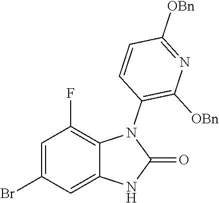 [SMILES: O=c1[nH]c2cc(Br)cc(F)c2n1-c1ccc(OCc2ccccc2)nc1OCc1ccccc1]

To a solution of 1-(2,6-bis(benzyloxy)pyridin-3-yl)-5-bromo-7-fluoro-1,3-dihydro-2H-benzo[d]imidazol-2-one (3.2 g, 6.16 mmol) in DMF (40 mL) were added K₂CO₃ (1.7 g, 12.32 mmol) and CH₃I (1.31 g, 9.24 mmol) at rt for overnight. The resulting mixture was quenched with water and extracted with EA. The organic layers were washed with brine. The organic layers were concentrated under vacuum. The residue was without purified to give the product (3.4 g, 99%) (90% purity) as light brown solid.

### Step 5: 3-(2,6-bis(benzyloxy)pyridin-3-yl)-6-(4-(dimethoxymethyl)piperidin-1-yl)-4-fluoro-1-methyl-1,3-dihydro-2H-benzo[d]imidazol-2-one

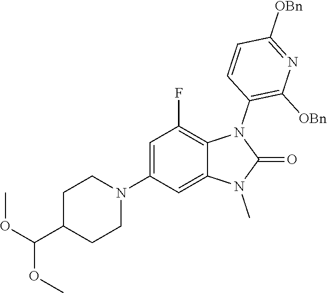 [SMILES: COC(OC)C1CCN(c2cc(F)c3c(c2)n(C)c(=O)n3-c2ccc(OCc3ccccc3)nc2OCc2ccccc2)CC1]

To a solution of 3-(2,6-bis(benzyloxy)pyridin-3-yl)-6-bromo-4-fluoro-1-methyl-1,3-dihydro-2H-benzo[d]imidazol-2-one (1.5 g, 2.81 mmol)(PH-BGUS-H-A613-5) in dry-dioxane (20 mL) were added 4-(dimethoxymethyl)piperidine (671 mg, 4.22 mmol), Pd-PEPPSI-IPentCl (118 mg, 0.14 mmol) and Cs₂CO₃ (1.83 g, 5.62 mmol) at 100° C. for 2 h under N₂ atmosphere. The resulting mixture was concentrated under vacuum. The residue was purified by combi-flash (PE/EA=100:0 to 70:30) to give the product (1.6 g, 93%) as light brown oil.

### Step 6: 3-(5-(4-(dimethoxymethyl)piperidin-1-yl)-7-fluoro-3-methyl-2-oxo-2,3-dihydro-1H-benzo[d]imidazol-1-yl)piperidine-2,6-dione

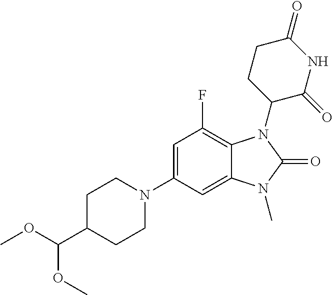 [SMILES: COC(OC)C1CCN(c2cc(F)c3c(c2)n(C)c(=O)n3C2CCC(=O)NC2=O)CC1]

To a solution of 3-(2,6-bis(benzyloxy)pyridin-3-yl)-6-(4-(dimethoxymethyl)piperidin-1-yl)-4-fluoro-1-methyl-1,3-dihydro-2H-benzo[d]imidazol-2-one (1.55 g, 2.53 mmol) in dry-THF (100 mL) was added Pd/C (1.55 g). The mixture was hydrogenated at room temperature for overnight under hydrogen atmosphere using a hydrogen balloon. The resulting mixture was filtered, the filter cake was washed with THF (100 mL). The filtrate was concentrated under vacuum. The residue was without purified to give the product (880 mg, 80%) as off-white solid. LCMS (ESI) m/e [M+1]⁺=435.

### Step 7: 1-(1-(2,6-dioxopiperidin-3-yl)-7-fluoro-3-methyl-2-oxo-2,3-dihydro-1H-benzo[d]imidazol-5-yl)piperidine-4-carbaldehyde

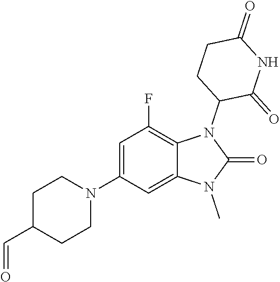 [SMILES: Cn1c(=O)n(C2CCC(=O)NC2=O)c2c(F)cc(N3CCC(C=O)CC3)cc21]

The titled compound was synthesized in the procedures similar to Example 37. LC-MS (ESI): m/z [M+H]⁺=389.6.

### Step 8: (1S,3R)-3-(3-((2-(1-((1-(1-((S)-2,6-dioxopiperidin-3-yl)-7-fluoro-3-methyl-2-oxo-2,3-dihydro-1H-benzo[d]imidazol-5-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

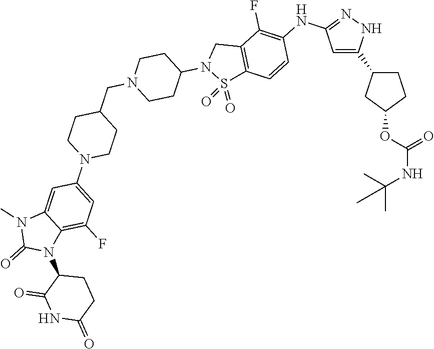 [SMILES: Cn1c(=O)n([C@H]2CCC(=O)NC2=O)c2c(F)cc(N3CCC(CN4CCC(N5Cc6c(ccc(Nc7cc([C@@H]8CC[C@H](OC(=O)NC(C)(C)C)C8)[nH]n7)c6F)S5(=O)=O)CC4)CC3)cc21]

The racemic product was synthesized in the procedures similar to Example 36, which was further separated by Chiral Prep-HPLC to afford the desired product:

(1S,3R)-3-(3-((2-(1-((1-(1-((S)-2,6-dioxopiperidin-3-yl)-7-fluoro-3-methyl-2-oxo-2,3-dihydro-1H-benzo[d]imidazol-5-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate (Example 41, 99.1% ee); Retention time: 7.24 min. ¹H NMR (500 MHz, DMSO-d₆) δ 11.98 (s, 1H), 11.07 (s, 1H), 8.77 (s, 1H), 8.29 (t, J=5 Hz, 1H), 7.51 (d, J=10 Hz, 1H), 6.76 (s, 1H), 6.68 (s, 1H), 6.55-6.49 (m, 1H), 5.83 (s, 1H), 5.50-5.43 (m, 1H), 4.98 (s, 1H), 4.52 (s, 2H), 3.70-3.61 (m, 2H), 3.47-3.39 (m, 1H), 3.32 (s, 3H), 3.25-2.88 (m, 4H), 2.67-2.55 (m, 5H), 2.35-2.15 (m, 3H), 2.10-1.95 (m, 4H), 1.90-1.56 (m, 10H), 1.30-1.17 (m, 11H). LC-MS (ESI): m/z [M+H]⁺=907.

Chiral analytical method: Column: i-Cellulose (4.6 mm×150 mm, 5 um); Mobile phase: A for MtBE (0.2% NH3·MeOH) and B for DCM:MeOH=50:50; Gradient: Mobile Phase A: Mobile Phase B=30:70 (v/v); HPLC Equipment: HPLC-Agilent, Back pressure: 100 bar; Column temperature: 35° C.

Chiral Prep-HPLC Condition: Column: i-Cellulose, 250*20.0 mm, 5 um; Mobile phase: A for MtBE (0.2% NH3·MeOH) and B for DCM:MeOH=50:50; Gradient: Mobile Phase A: Mobile Phase B=30:70 (v/v); Flow Rate: 20 mL/min, Wave Length: UV 200 nm and 270 nm, Prep-HPLC Equipment: Prep-HPLC-Gilson, Back pressure: 100 bar; Column temperature: 25° C.

### Example 42: (1S,3R)-3-(3-((2-(1-((1-(1-(2,6-dioxopiperidin-3-yl)-3,4-dimethyl-2-oxo-2,3-dihydro-1H-benzo[d]imidazol-5-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

### Step 1: 5-bromo-N¹,6-dimethylbenzene-1,2-diamine

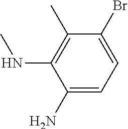

3-bromo-N,2-dimethyl-6-nitroaniline (2 g, 8.16 mmol) was dissolved in THF (30 mL). Raney-Ni (2 g, wet) was added to the solution in one portion. The resulting mixture was stirred under hydrogen atmosphere (1 atm) at room temperature for 3 h. The solid was filtered off and the filtrate was concentrated to give the crude product. The residue was purified by silica gel column chromatography, eluted with DMC/MeOH (40:1) to afford the product (1.4 g, 80%); LC-MS (ESI): m/z [M+H]⁺=215.2.

### Step 2: 5-bromo-N¹,6-dimethylbenzene-1,2-diamine

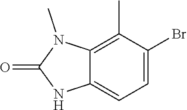

To a solution of 5-bromo-N,6-dimethylbenzene-1,2-diamine (1.4 g, 6.5 mmol) in MeCN (20 mL) was added CDI (1.58 g, 9.8 mmol). The resulting mixture was stirred at 80° C. for 2 h. The resulting mixture was filtered to afford the product (1.1 g, 70% yield). LC-MS (ESI): m/z [M+H]⁺=241.3.

### Step 3: 6-(4-(dimethoxymethyl)piperidin-1-yl)-1,7-dimethyl-1,3-dihydro-2H-benzo[d]imidazol-2-one

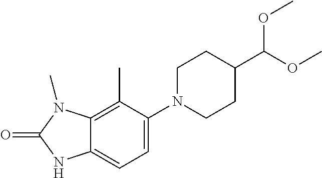 [SMILES: COC(OC)C1CCN(c2ccc3[nH]c(=O)n(C)c3c2C)CC1]

To a mixture of 6-bromo-1,7-dimethyl-1,3-dihydro-2H-benzo[d]imidazol-2-one (500 mg, 2 mmol) and 4-(dimethoxymethyl)piperidine (500 mg, 2.07 mmol) in andydrous THF (15 mL) was added Pd₂(dba)₃ (380 mg, 0.41 mmol), RuPhos (387 mg, 0.83 mmol) and LiHMDS (8.3 mL, 8.3 mmol, 1 M in THF) at 0° C. The reaction mixture was stirred under nitrogen atmosphere at 70° C. for 1 h. The mixture was diluted with EA (100 mL), washed with NH₄Cl aq. (2×50 mL) and brine (50 mL). The organic layer was dried over anhydrous Na₂SO₄, filtered and concentrated under reduced pressure. The residue was purified by silica gel column chromatography, eluting with DCM/MeOH (1:30) to afford the product (360 mg, 54% yield). LC-MS (ESI): m/z [M+H]⁺=320.4.

### Step 4: 3-(5-(4-(dimethoxymethyl)piperidin-1-yl)-3,4-dimethyl-2-oxo-2,3-dihydro-1H-benzo[d]imidazol-1-yl)piperidine-2,6-dione

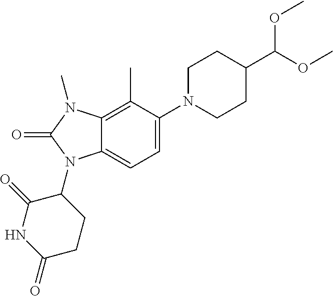 [SMILES: COC(OC)C1CCN(c2ccc3c(c2C)n(C)c(=O)n3C2CCC(=O)NC2=O)CC1]

To a stirring solution of 6-(4-(dimethoxymethyl)piperidin-1-yl)-1,7-dimethyl-1,3-dihydro-2H-benzo[d]imidazol-2-one (360 mg, 1.13 mmol) and 3-bromopiperidine-2,6-dione (432 mg, 2.25 mmol) in THF (10 mL) was added NaH (225 mg, 5.64 mmol, 60% in mineral oil) slowly at 0° C. The resulting solution was heated to 70° C. for 2 h. The solution was cooled to 0° C., quenched with NH₄Cl aq. (50 mL). The mixture was extracted with DCM (100 mL×3). The combined organic layers were washed with brine (50 mL) and dried over anhydrous Na₂SO₄. After filtration, the filtrate was concentrated under reduced pressure. The residue was purified by silica gel column chromatography, eluting with DCM/MeOH (1:30) to afford the product (350 mg, 72% yield). LC-MS (ESI): m/z [M+H]⁺=431.5.

### Step 5: 1-(1-(2,6-dioxopiperidin-3-yl)-3,4-dimethyl-2-oxo-2,3-dihydro-1H-benzo[d]imidazol-5-yl)piperidine-4-carbaldehyde

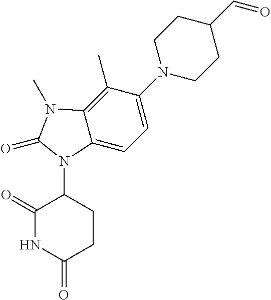 [SMILES: Cc1c(N2CCC(C=O)CC2)ccc2c1n(C)c(=O)n2C1CCC(=O)NC1=O]

To a mixture of 3-(5-(4-(dimethoxymethyl)piperidin-1-yl)-3,4-dimethyl-2-oxo-2,3-dihydro-1H-benzo[d]imidazol-1-yl)piperidine-2,6-dione (350 mg, 0.81 mmol) in HCl aq (10 mL, 8 N) was stirred at room temperature for 3 h. The solution was cooled to 0° C., quenched with NaHCO₃ aq. (150 mL). The mixture was extracted with DCM (100 mL×3). The combined organic layers were washed with brine (50 mL) and dried over anhydrous Na₂SO₄. After filtration, the filtrate was concentrated under reduced pressure to afford the product (280 mg, 90% yield). LC-MS (ESI): m/z [M+H]⁺=385.4.

### Step 6: (1S,3R)-3-(3-((2-(1-((1-(1-(2,6-dioxopiperidin-3-yl)-3,4-dimethyl-2-oxo-2,3-dihydro-1H-benzo[d]imidazol-5-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

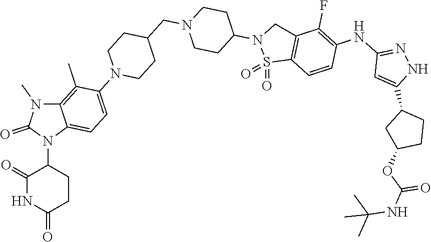 [SMILES: Cc1c(N2CCC(CN3CCC(N4Cc5c(ccc(Nc6cc([C@@H]7CC[C@H](OC(=O)NC(C)(C)C)C7)[nH]n6)c5F)S4(=O)=O)CC3)CC2)ccc2c1n(C)c(=O)n2C1CCC(=O)NC1=O]

The titled compound was synthesized in the procedures similar to Example 36. ¹H NMR (500 MHz, DMSO) δ 11.06 (s, 1H), 8.77 (s, 1H), 8.28 (s, 1H), 7.51 (d, J=8.7 Hz, 1H), 6.86 (d, J=8.4 Hz, 1H), 6.78 (d, J=8.5 Hz, 2H), 5.83 (s, 1H), 5.30 (dd, J=12.6, 5.3 Hz, 1H), 4.98 (s, 1H), 4.52 (s, 2H), 3.58 (s, 3H), 3.43-3.41 (m, 1H), 3.11-3.04 (m, 1H), 2.97-2.84 (m, 5H), 2.72-2.53 (m, 8H), 2.47-2.44 (m, 1H), 2.23-2.21 (m, 2H), 2.08-1.95 (m, 4H), 1.92-1.87 (m, 5H), 1.82-1.97 (m, 2H), 1.72 (d, J=8.2 Hz, 2H), 1.60 (s, 2H), 1.30-1.28 (m, 2H), 1.21 (s, 9H). LC-MS (ESI): m/z [M+H]⁺=903.54.

### Example 43: (1S,3R)-3-(3-((2-(1′-(1-(2,6-dioxopiperidin-3-yl)-3,4-dimethyl-2-oxo-2,3-dihydro-1H-benzo[d]imidazol-5-yl)-[1,4′-bipiperidin]-4-yl)-4-fluoro-1,1-dioxido-2,3-dihydrobenzo[d]isothiazol-5-yl)amino)-1H-pyrazol-5-yl)cyclopentyl tert-butylcarbamate

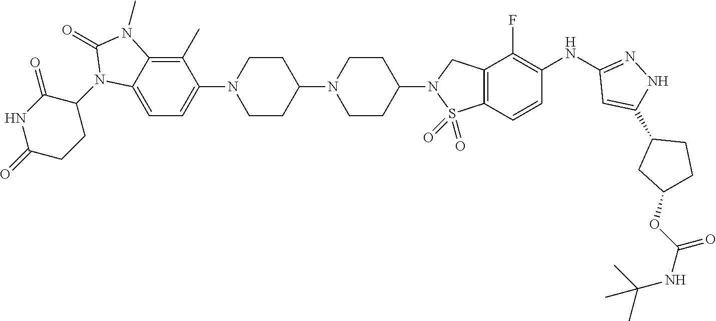 [SMILES: Cc1c(N2CCC(N3CCC(N4Cc5c(ccc(Nc6cc([C@@H]7CC[C@H](OC(=O)NC(C)(C)C)C7)[nH]n6)c5F)S4(=O)=O)CC3)CC2)ccc2c1n(C)c(=O)n2C1CCC(=O)NC1=O]

### Step 1: 5-(4-(dimethoxymethyl)piperidin-1-yl)-3-methyl-1,3-dihydro-2H-imidazo[4,5-b]pyridin-2-one

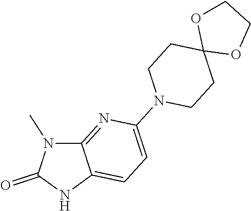 [SMILES: Cn1c(=O)[nH]c2ccc(N3CCC4(CC3)OCCO4)nc21]

To a mixture of 5-chloro-3-methyl-1,3-dihydro-2H-imidazo[4,5-b]pyridin-2-one (500 mg, 2.72 mmol) and 1,4-dioxa-8-azaspiro[4.5]decane (585 mg, 4.09 mmol) in andydrous THF (15 mL) was added Pd2(dba)3 (500 mg, 0.54 mmol), RuPhos (508 mg, 1 mmol) and LiHMDS (10.9 mL, 10.9 mmol, 1 M in THF) at 0° C. The reaction mixture was stirred under nitrogen atmosphere at 70° C. for 1 h. The mixture was diluted with EA (100 mL), washed with NH₄Cl aq. (2×50 mL) and brine (50 mL). The organic layer was dried over anhydrous Na₂SO₄, filtered and concentrated under reduced pressure. The residue was purified by silica gel column chromatography, eluting with DCM/MeOH (1:30) to afford the product (490 mg, 62% yield). LC-MS (ESI): m/z [M+H]⁺=291.5.

### Step 2: 3-(3-methyl-2-oxo-5-(1,4-dioxa-8-azaspiro[4.5]decan-8-yl)-2,3-dihydro-1H-imidazo[4,5-b]pyridin-1-yl)piperidine-2,6-dione

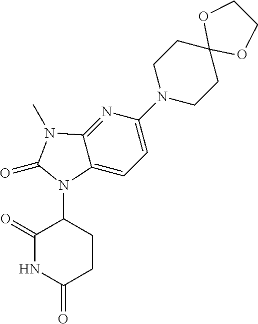 [SMILES: Cn1c(=O)n(C2CCC(=O)NC2=O)c2ccc(N3CCC4(CC3)OCCO4)nc21]

To a stirring solution of 3-methyl-5-(1,4-dioxa-8-azaspiro[4.5]decan-8-yl)-1,3-dihydro-2H-imidazo[4,5-b]pyridin-2-one (490 mg, 1.69 mmol) and 3-bromopiperidine-2,6-dione (648 mg, 3.38 mmol) in THF (12 mL) was added NaH (270 mg, 6.75 mmol, 60% in mineral oil) slowly at 0° C. The resulting solution was heated to 70° C. for 2 h. The solution was cooled to 0° C., quenched with NH₄Cl aq. (50 mL). The mixture was extracted with DCM (100 mL×3). The combined organic layers were washed with brine (50 mL) and dried over anhydrous Na₂SO₄. After filtration, the filtrate was concentrated under reduced pressure. The residue was purified by silica gel column chromatography, eluting with DCM/MeOH (1:30) to afford the product (480 mg, 71% yield). LC-MS (ESI): m/z [M+H]⁺=402.3.

### Step 3: 3-(3-methyl-2-oxo-5-(4-oxopiperidin-1-yl)-2,3-dihydro-1H-imidazo[4,5-b]pyridin-1-yl)piperidine-2,6-dione

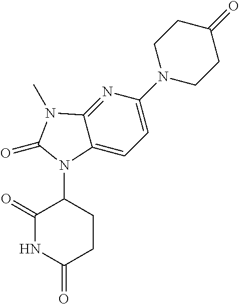 [SMILES: Cn1c(=O)n(C2CCC(=O)NC2=O)c2ccc(N3CCC(=O)CC3)nc21]

To a mixture of 3-(3-methyl-2-oxo-5-(1,4-dioxa-8-azaspiro[4.5]decan-8-yl)-2,3-dihydro-1H-imidazo[4,5-b]pyridin-1-yl)piperidine-2,6-dione (480 mg, 1.2 mmol) in HCl aq (15 mL, 8 N) was stirred at room temperature for 3 h. The solution was cooled to 0° C., quenched with NaHCO₃ aq. (200 mL). The mixture was extracted with DCM (100 mL×3). The combined organic layers were washed with brine (50 mL) and dried over anhydrous Na₂SO₄. After filtration, the filtrate was concentrated under reduced pressure to afford the product (390 mg, 91% yield). LC-MS (ESI): m/z [M+H]⁺=358.4.

### Step 4: (1S,3R)-3-(3-((2-(1′-(1-(2,6-dioxopiperidin-3-yl)-3,4-dimethyl-2-oxo-2,3-dihydro-1H-benzo[d]imidazol-5-yl)-[1,4′-bipiperidin]-4-yl)-4-fluoro-1,1-dioxido-2,3-dihydrobenzo[d]isothiazol-5-yl)amino)-1H-pyrazol-5-yl)cyclopentyl tert-butylcarbamate

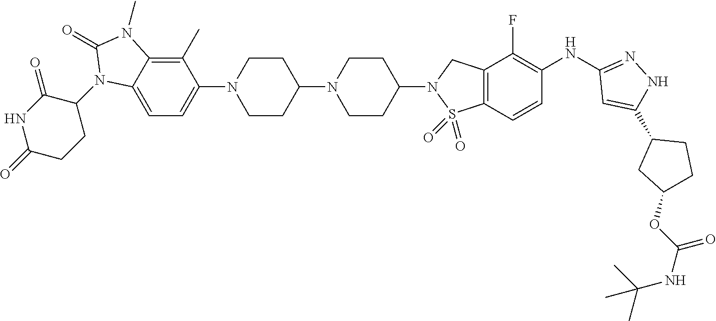 [SMILES: Cc1c(N2CCC(N3CCC(N4Cc5c(ccc(Nc6cc([C@@H]7CC[C@H](OC(=O)NC(C)(C)C)C7)[nH]n6)c5F)S4(=O)=O)CC3)CC2)ccc2c1n(C)c(=O)n2C1CCC(=O)NC1=O]

The titled compound was synthesized in the procedures similar to Example 36. ¹H NMR (500 MHz, DMSO) δ 11.07 (s, 1H), 9.39 (d, J=61.3 Hz, 1H), 8.82 (s, 1H), 8.33-8.28 (m, 1H), 7.56-7.54 (m, 1H), 6.89 (d, J=8.0 Hz, 1H), 6.8-6.75 (m, 2H), 5.84 (s, 1H), 5.33-5.31 (m, 1H), 4.98 (s, 1H), 4.58 (s, 2H), 3.80 (d, J=54.7 Hz, 1H), 3.65-3.59 (m, 3H), 3.48 (s, 1H), 3.35 (s, 1H), 3.25 (d, J=8.4 Hz, 2H), 3.08-3.07 (m, 3H), 2.92-2.86 (m, 1H), 2.69-2.60 (m, 5H), 2.56-2.54 (m, 3H), 2.47-2.40 (m, 1H), 2.27-2.25 (m, 2H), 2.21-2.09 (m, 3H), 2.08-1.94 (m, 3H), 1.92-1.80 (m, 3H), 1.73-1.68 (m, 2H), 1.59 (s, 1H), 1.21 (s, 9H). LC-MS (ESI): m/z [M+H]⁺=889.57.

### Example 44: (1S,3R)-3-(3-((2-(1-(1-(1-(2,6-dioxopiperidin-3-yl)-4-fluoro-3-methyl-2-oxo-2,3-dihydro-1H-benzo[d]imidazol-5-yl)azetidin-3-yl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

### Step 1: 1-(2,6-bis(benzyloxy)pyridin-3-yl)-4-fluoro-3-methyl-5-(5,8-dioxa-2-azaspiro[3.4]octan-2-yl)-1,3-dihydro-2H-benzo[d]imidazol-2-one

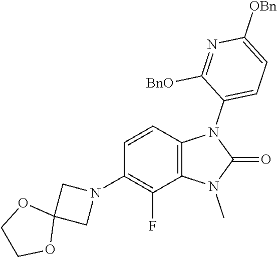 [SMILES: Cn1c(=O)n(-c2ccc(OCc3ccccc3)nc2OCc2ccccc2)c2ccc(N3CC4(C3)OCCO4)c(F)c21]

Into a 250-mL round-bottom flask purged and maintained with N₂, were added 1-(2,6-bis(benzyloxy)pyridin-3-yl)-5-bromo-4-fluoro-3-methyl-1,3-dihydro-2H-benzo[d]imidazol-2-one (1.60 g, 3.00 mmol), 5,8-dioxa-2-azaspiro[3.4]octane hydrochloride (0.54 g, 3.60 mmol), Cs₂CO₃ (2.93 g, 9.00 mmol), Pd-PEPPSI2Me-IPENT Cl (126.0 mg, 0.15 mmol) and Dioxane (50 mL) at room temperature. The resulting mixture was stirred 2 h at 100° C. After filtration, the filtrate was concentrated under reduced pressure. The residue was purified by silica gel column chromatography, eluted with PE/EtOAc (100:0-70:30) to afford 1-(2,6-bis(benzyloxy)pyridin-3-yl)-4-fluoro-3-methyl-5-(5,8-dioxa-2-azaspiro[3.4]octan-2-yl)-1,3-dihydro-2H-benzo[d]imidazol-2-one (1.00 g, 58.65%) as yellow solid.

### Step 2: 3-(4-fluoro-3-methyl-2-oxo-5-(5,8-dioxa-2-azaspiro[3.4]octan-2-yl)-2,3-dihydro-1H-benzo[d]imidazol-1-yl)piperidine-2,6-dione

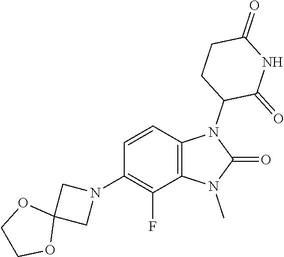 [SMILES: Cn1c(=O)n(C2CCC(=O)NC2=O)c2ccc(N3CC4(C3)OCCO4)c(F)c21]

Into a 250-mL round-bottom flask purged and maintained with H₂, were added 1-(2,6-bis(benzyloxy)pyridin-3-yl)-4-fluoro-3-methyl-5-(5,8-dioxa-2-azaspiro[3.4]octan-2-yl)-1,3-dihydro-2H-benzo[d]imidazol-2-one (900.0 mg, 1.58 mmol), Pd/C (900.0 mg) and THF (30 mL) at room temperature. The resulting mixture was stirred 15 h at room temperature. The precipitated solids were collected by filtration and washed with THF (300 mL). After filtration, the filtrate was concentrated under reduced pressure. The residue was purified by silica gel column chromatography, eluted with PE/EtOAc (100:0-50:50) to afford 3-(4-fluoro-3-methyl-2-oxo-5-(5,8-dioxa-2-azaspiro[3.4]octan-2-yl)-2,3-dihydro-1H-benzo[d]imidazol-1-yl)piperidine-2,6-dione (600.0 mg, 97.09%) as yellow solid.

### Step 3: 3-(4-fluoro-3-methyl-2-oxo-5-(3-oxoazetidin-1-yl)-2,3-dihydro-1H-benzo[d]imidazol-1-yl)piperidine-2,6-dione

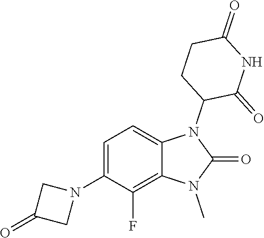 [SMILES: Cn1c(=O)n(C2CCC(=O)NC2=O)c2ccc(N3CC(=O)C3)c(F)c21]

Into a 250-mL round-bottom flask were added 3-(4-fluoro-3-methyl-2-oxo-5-(5,8-dioxa-2-azaspiro[3.4]octan-2-yl)-2,3-dihydro-1H-benzo[d]imidazol-1-yl)piperidine-2,6-dione (700.0 mg, 1.79 mmol) and H₂SO₄ (10 mL) at 0° C. The resulting mixture was stirred 2 h at room temperature. The reaction was quenched by the addition of ice water (50 mL). The residue was acidified to PH=7 with Na₂CO₃ solution. The resulting mixture was extracted with EtOAc (2×100 mL), dried over anhydrous Na₂SO₄. The resulting mixture was concentrated under reduced pressure to afford 3-(4-fluoro-3-methyl-2-oxo-5-(3-oxoazetidin-1-yl)-2,3-dihydro-1H-benzo[d]imidazol-1-yl)piperidine-2,6-dione (533.7 mg, 85.94%) as light grey solid.

### Step 4: (1S,3R)-3-(3-((2-(1-(1-(1-(2,6-dioxopiperidin-3-yl)-4-fluoro-3-methyl-2-oxo-2,3-dihydro-1H-benzo[d]imidazol-5-yl)azetidin-3-yl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

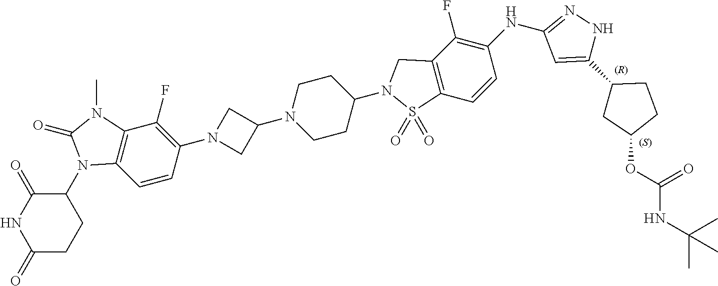

The titled compound was synthesized in the procedures similar to Example 39. ¹H NMR (500 MHz, DMSO) δ 12.00 (s, 1H), 11.09 (s, 1H), 8.79 (d, J=20.8 Hz, 1H), 8.30 (s, 1H), 7.53 (s, 1H), 6.90-6.70 (m, 2H), 6.30 (s, 1H), 5.83 (s, 1H), 5.31 (s, 1H), 4.98 (s, 1H), 4.53 (d, J=14.5 Hz, 2H), 4.16 (s, 2H), 4.00 (s, 1H), 3.67-3.53 (m, 2H), 3.45 (s, 4H), 3.28-3.18 (m, 1H), 3.06 (s, 2H), 2.94-2.81 (m, 2H), 2.72-2.54 (m, 3H), 2.22 (s, 2H), 2.08-1.81 (m, 6H), 1.76-1.66 (m, 2H), 1.58 (s, 1H), 1.21 (s, 9H). LC-MS (ESI): m/z [M+H]⁺=845.61

### Example 45: (1S,3R)-3-(3-((2-(1-(1-(1-(2,6-dioxopiperidin-3-yl)-4-fluoro-3-methyl-2-oxo-2,3-dihydro-1H-benzo[d]imidazol-5-yl)piperidin-4-yl)azetidin-3-yl)-4-fluoro-1,1-dioxido-2,3-dihydrobenzo[d]isothiazol-5-yl)amino)-1H-pyrazol-5-yl)cyclopentyl tert-butylcarbamate

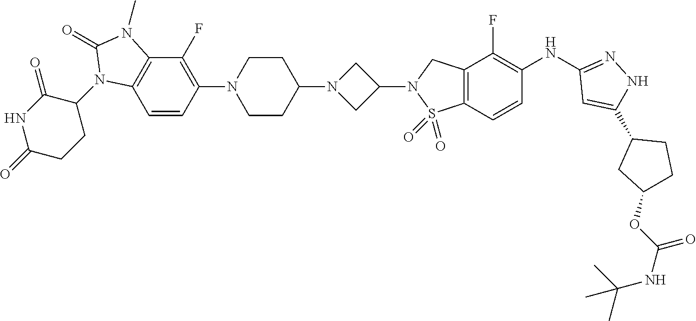 [SMILES: Cn1c(=O)n(C2CCC(=O)NC2=O)c2ccc(N3CCC(N4CC(N5Cc6c(ccc(Nc7cc([C@@H]8CC[C@H](OC(=O)NC(C)(C)C)C8)[nH]n7)c6F)S5(=O)=O)C4)CC3)c(F)c21]

### Step 1: 3-(4-fluoro-3-methyl-2-oxo-5-(4-oxopiperidin-1-yl)-2,3-dihydro-1H-benzo[d]imidazol-1-yl)piperidine-2,6-dione

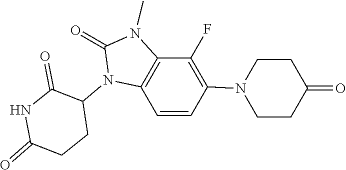 [SMILES: Cn1c(=O)n(C2CCC(=O)NC2=O)c2ccc(N3CCC(=O)CC3)c(F)c21]

The titled compound was synthesized in the procedures similar to Example 44. LC-MS (ESI): m/z [M+H]⁺=375.6.

### Step 2: (1S,3R)-3-(3-((2-(1-(1-(1-(2,6-dioxopiperidin-3-yl)-4-fluoro-3-methyl-2-oxo-2,3-dihydro-1H-benzo[d]imidazol-5-yl)piperidin-4-yl)azetidin-3-yl)-4-fluoro-1,1-dioxido-2,3-dihydrobenzo[d]isothiazol-5-yl)amino)-1H-pyrazol-5-yl)cyclopentyl tert-butylcarbamate

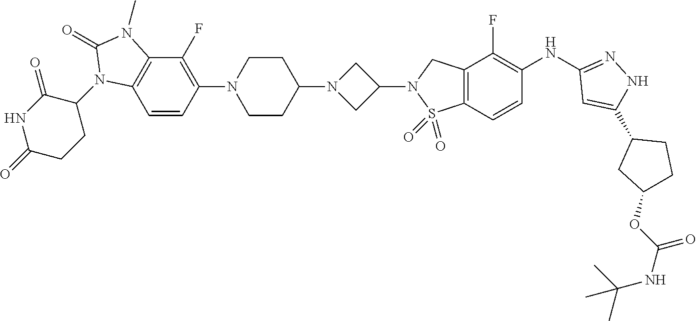 [SMILES: Cn1c(=O)n(C2CCC(=O)NC2=O)c2ccc(N3CCC(N4CC(N5Cc6c(ccc(Nc7cc([C@@H]8CC[C@H](OC(=O)NC(C)(C)C)C8)[nH]n7)c6F)S5(=O)=O)C4)CC3)c(F)c21]

The titled compound was synthesized in the procedures similar to Example 39. ¹H NMR (500 MHz, DMSO) δ 11.99 (s, 1H), 11.10 (s, 1H), 8.82 (s, 1H), 8.30 (s, 1H), 7.54 (d, J=8.7 Hz, 1H), 6.85 (d, J=8.6 Hz, 1H), 6.81-6.67 (m, 2H), 5.84 (s, 1H), 5.35-5.31 (m, 1H), 4.98 (s, 1H), 4.61 (s, 2H), 4.29-4.10 (m, 1H), 3.54-3.51 (m, 2H), 3.47 (s, 3H), 3.19 (d, J=11.4 Hz, 2H), 3.10-3.03 (m, 1H), 2.91-2.85 (m, 1H), 2.77-2.53 (m, 4H), 2.48-2.44 (m, 2H), 2.26 (s, 1H), 2.02-2.00 (m, 2H), 1.95-1.85 (m, 1H), 1.82-1.65 (m, 4H), 1.58 (s, 1H), 1.38-1.36 (m, 2H), 1.21 (s, 9H). LC-MS (ESI): m/z [M+H]⁺=865.53.

### Example 46: (1S,3R)-3-(3-((2-(1-((1-(1-(2,6-dioxopiperidin-3-yl)-4-fluoro-3-methyl-2-oxo-2,3-dihydro-1H-benzo[d]imidazol-5-yl)piperidin-4-yl)methyl)azetidin-3-yl)-4-fluoro-1,1-dioxido-2,3-dihydrobenzo[d]isothiazol-5-yl)amino)-1H-pyrazol-5-yl)cyclopentyl tert-butylcarbamate

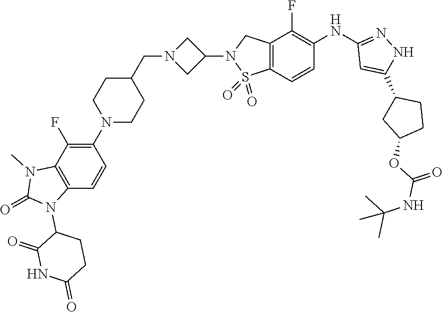 [SMILES: Cn1c(=O)n(C2CCC(=O)NC2=O)c2ccc(N3CCC(CN4CC(N5Cc6c(ccc(Nc7cc([C@@H]8CC[C@H](OC(=O)NC(C)(C)C)C8)[nH]n7)c6F)S5(=O)=O)C4)CC3)c(F)c21]

The titled compound was synthesized in the procedures similar to Example 36. ¹H NMR (500 MHz, DMSO) δ 12.00 (s, 1H), 11.09 (s, 1H), 8.81 (s, 1H), 8.29 (s, 1H), 7.53 (d, J=8.6 Hz, 1H), 6.84 (d, J=8.5 Hz, 1H), 6.76-6.71 (m, 2H), 5.84 (s, 1H), 5.32 (dd, J=12.8, 5.3 Hz, 1H), 4.98 (s, 1H), 4.62 (s, 2H), 4.14 (p, J=6.3 Hz, 1H), 3.53-3.5 (m, 2H), 3.47-3.46 (m, 3H), 3.23-3.2 (m, 2H), 3.10-3.00 (m, 1H), 2.91-2.85 (m, 1H), 2.72-2.54 (m, 5H), 2.48-2.44 (m, 2H), 2.40-2.39 (m, 2H), 2.06-1.96 (m, 2H), 1.95-1.85 (m, 1H), 1.83-1.67 (m, 4H), 1.58 (s, 1H), 1.41 (s, 1H), 1.36-1.26 (m, 2H), 1.21 (s, 9H). LC-MS (ESI): m/z [M+H]⁺=879.57.

### Example 47: (1S,3R)-3-(3-((2-(1-((1-(1-(2,6-dioxopiperidin-3-yl)-4-fluoro-3-methyl-2-oxo-2,3-dihydro-1H-benzo[d]imidazol-5-yl)piperidin-4-yl)methyl)-4-methylpiperidin-4-yl)-4-fluoro-1,1-dioxido-2,3-dihydrobenzo[d]isothiazol-5-yl)amino)-1H-pyrazol-5-yl)cyclopentyl tert-butylcarbamate

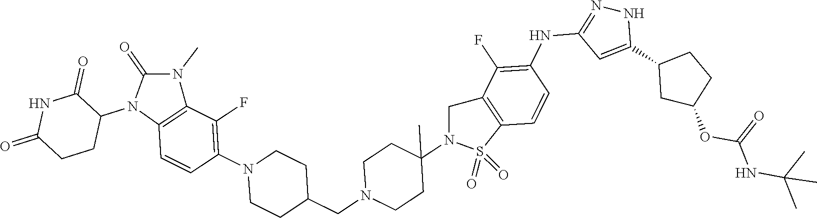 [SMILES: Cn1c(=O)n(C2CCC(=O)NC2=O)c2ccc(N3CCC(CN4CCC(C)(N5Cc6c(ccc(Nc7cc([C@@H]8CC[C@H](OC(=O)NC(C)(C)C)C8)[nH]n7)c6F)S5(=O)=O)CC4)CC3)c(F)c21]

### Step 1: tert-butyl 4-((4-bromo-3-fluoro-2-(methoxycarbonyl)phenyl)sulfonamido)-4-methylpiperidine-1-carboxylate

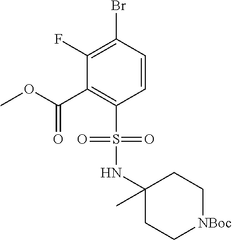 [SMILES: COC(=O)c1c(S(=O)(=O)NC2(C)CCN(C(=O)OC(C)(C)C)CC2)ccc(Br)c1F]

To a solution of methyl 3-bromo-6-(chlorosulfonyl)-2-fluorobenzoate (5 g, 15.19 mmol) in ACN (50 mL), H2O (10 mL) was added tert-butyl 4-amino-4-methylpiperidine-1-carboxylate (4.3 g, 18.23 mmol), and Na2CO3 (3.22 g, 30.38 mmol). The reaction mixture was stirred for 3 hours at rt. The reaction was quenched by the addition of water (100 mL). The resulting mixture was extracted with EtOAc (2×100 mL), dried over anhydrous Na₂SO₄. The resulting mixture was concentrated under reduced pressure purified by silica gel column chromatography, eluting with PE/EA (1:3) to afford the product (6.4 g, 80%). LC-MS (ESI): m/z [M+H]⁺=509.2.

### Step 2: tert-butyl 4-((4-bromo-3-fluoro-2-(hydroxymethyl)phenyl)sulfonamido)-4-methylpiperidine-1-carboxylate

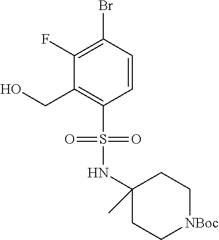 [SMILES: CC1(NS(=O)(=O)c2ccc(Br)c(F)c2CO)CCN(C(=O)OC(C)(C)C)CC1]

To a solution of tert-butyl 4-((4-bromo-3-fluoro-2-(methoxycarbonyl)phenyl) sulfonamido)-4-methylpiperidine-1-carboxylate (5 g, 9.43 mmol) a in THF (70 mL) was added LiAlH4 (2.5M) (4.5 ml, 11.316 mmol) at −15° C. under N₂ atmosphere. The reaction mixture was stirred for 2 hours at −15° C. The reaction was quenched by the addition of Sodium sulfate decahydrate. The resulting mixture was concentrated under reduced pressure purified by silica gel column chromatography, eluting with PE/EA (1:1) to afford the product (3 g, 63.8%). LC-MS (ESI): m/z [M+H]⁺=481.2.

### Step 3: tert-butyl 4-((4-bromo-2-(bromomethyl)-3-fluorophenyl)sulfonamido)-4-methylpiperidine-1-carboxylate

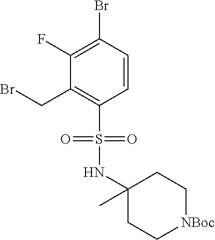 [SMILES: CC1(NS(=O)(=O)c2ccc(Br)c(F)c2CBr)CCN(C(=O)OC(C)(C)C)CC1]

To a solution of tert-butyl 4-((4-bromo-3-fluoro-2-(hydroxymethyl)phenyl)sulfonamido)-4-methylpiperidine-1-carboxylate (3 g, 5.97 mmol) and Carbon tetrabromide (2.96 g, 8.96 mmol) in DCM was added triphenylphosphine (3.14 g, 11.94 mmol), in portions at 0° C. The reaction mixture was stirred for overnight at 40° C. The mixture was concentrated under reduced pressure. The residue was purified by silica gel column chromatography, eluting with PE/EA (1:1) to afford the product (2.5 g, 75%). LC-MS (ESI): m/z [M+H]⁺=543.2.

### Step 4: tert-butyl 4-(5-bromo-4-fluoro-1,1-dioxidobenzo[d]isothiazol-2(3H)-yl)-4-methylpiperidine-1-carboxylate

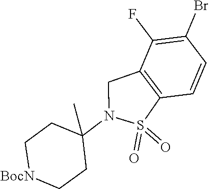 [SMILES: CC(C)(C)OC(=O)N1CCC(C)(N2Cc3c(ccc(Br)c3F)S2(=O)=O)CC1]

To a solution of tert-butyl 4-((4-bromo-2-(bromomethyl)-3-fluorophenyl)sulfonamido)-4-methylpiperidine-1-carboxylate (2.5 g, 4.44 mmol) in ACN (30 mL), H₂O (5 mL) was added Na₂CO₃ (3.22 g, 30.38 mmol). The reaction mixture was stirred for 3 hours at 80° C. The reaction was quenched by the addition of water (100 mL). The resulting mixture was extracted with EtOAc (2×100 mL), dried over anhydrous Na₂SO₄. The resulting mixture was concentrated under reduced pressure purified by silica gel column chromatography, eluting with PE/EA (1:2) to afford the product (1.7 g, 81%). LC-MS (ESI): m/z [M+H]⁺=463.2.

### Step 5: (1S,3R)-3-(3-((2-(1-((1-(1-(2,6-dioxopiperidin-3-yl)-4-fluoro-3-methyl-2-oxo-2,3-dihydro-1H-benzo[d]imidazol-5-yl)piperidin-4-yl)methyl)-4-methylpiperidin-4-yl)-4-fluoro-1,1-dioxido-2,3-dihydrobenzo[d]isothiazol-5-yl)amino)-1H-pyrazol-5-yl)cyclopentyl tert-butylcarbamate

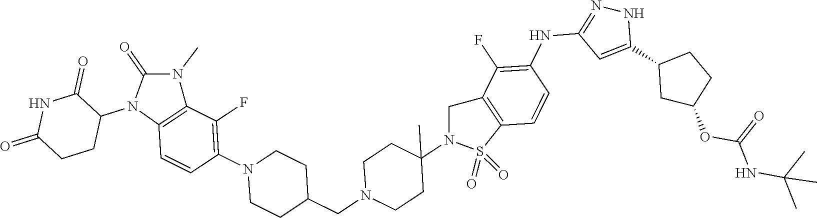 [SMILES: Cn1c(=O)n(C2CCC(=O)NC2=O)c2ccc(N3CCC(CN4CCC(C)(N5Cc6c(ccc(Nc7cc([C@@H]8CC[C@H](OC(=O)NC(C)(C)C)C8)[nH]n7)c6F)S5(=O)=O)CC4)CC3)c(F)c21]

The titled compound was synthesized in the procedures similar to Example 36. ¹H NMR (500 MHz, DMSO) δ 11.96 (s, 1H), 11.09 (s, 1H), 8.75 (s, 1H), 8.30 (s, 1H), 8.17 (s, 1H), 7.45 (d, J=8.5 Hz, 1H), 6.84 (d, J=8.5 Hz, 1H), 6.80-6.70 (m, 2H), 5.83 (s, 1H), 5.32 (dd, J=12.8, 5.1 Hz, 1H), 5.06-4.96 (m, 1H), 4.51 (s, 2H), 3.47 (s, 3H), 3.22 (d, J=10.0 Hz, 2H), 3.10-3.01 (m, 1H), 2.94-2.84 (m, 2H), 2.72-2.64 (m, 4H), 2.45-2.41 (m, 1H), 2.28-2.14 (m, 4H), 2.06-1.96 (m, 2H), 1.94-1.84 (m, 1H), 1.84-1.67 (m, 7H), 1.66-1.54 (m, 2H), 1.40 (s, 3H), 1.34-1.24 (m, 3H), 1.21 (s, 9H). LC-MS (ESI): m/z [M+H]⁺=921.5

### Example 48: (1S,3R)-3-(3-((2-(1′-(1-(2,6-dioxopiperidin-3-yl)-4-fluoro-3-methyl-2-oxo-2,3-dihydro-1H-benzo[d]imidazol-5-yl)-4-methyl-[1,4′-bipiperidin]-4-yl)-4-fluoro-1,1-dioxido-2,3-dihydrobenzo[d]isothiazol-5-yl)amino)-1H-pyrazol-5-yl)cyclopentyl tert-butylcarbamate

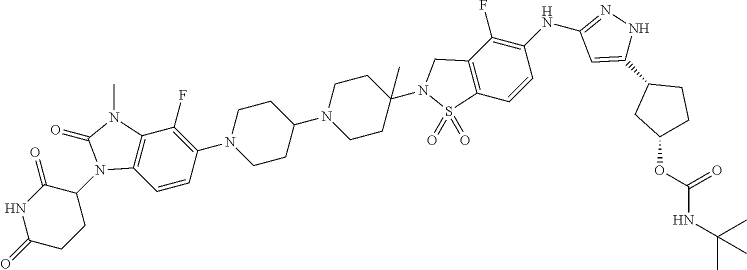 [SMILES: Cn1c(=O)n(C2CCC(=O)NC2=O)c2ccc(N3CCC(N4CCC(C)(N5Cc6c(ccc(Nc7cc([C@@H]8CC[C@H](OC(=O)NC(C)(C)C)C8)[nH]n7)c6F)S5(=O)=O)CC4)CC3)c(F)c21]

The titled compound was synthesized in the procedures similar to Example 39. ¹H NMR (500 MHz, DMSO) δ 11.96 (s, 1H), 11.09 (s, 1H), 8.75 (s, 1H), 8.30 (s, 1H), 8.17 (s, 1H), 7.45 (d, J=8.5 Hz, 1H), 6.84 (d, J=8.5 Hz, 1H), 6.80-6.70 (m, 2H), 5.83 (s, 1H), 5.32 (dd, J=12.8, 5.1 Hz, 1H), 5.06-4.96 (m, 1H), 4.51 (s, 2H), 3.47 (s, 3H), 3.22 (d, J=10.0 Hz, 2H), 3.10-3.01 (m, 1H), 2.94-2.84 (m, 2H), 2.72-2.64 (m, 4H), 2.45-2.41 (m, 1H), 2.28-2.14 (m, 4H), 2.06-1.96 (m, 2H), 1.94-1.84 (m, 1H), 1.84-1.67 (m, 5H), 1.66-1.54 (m, 2H), 1.40 (s, 3H), 1.34-1.24 (m, 3H), 1.21 (s, 9H). LC-MS (ESI): m/z [M+H]⁺=907.5;

### Example 49: (1S,3R)-3-(3-((2-((R)-1-((1-(1-((S)-2,6-dioxopiperidin-3-yl)-7-fluoro-3-methyl-2-oxo-2,3-dihydro-1H-benzo[d]imidazol-5-yl)piperidin-4-yl)methyl)-2,2-dimethylpiperidin-4-yl)-4-fluoro-1,1-dioxido-2,3-dihydrobenzo[d]isothiazol-5-yl)amino)-1H-pyrazol-5-yl)cyclopentyl tert-butylcarbamate

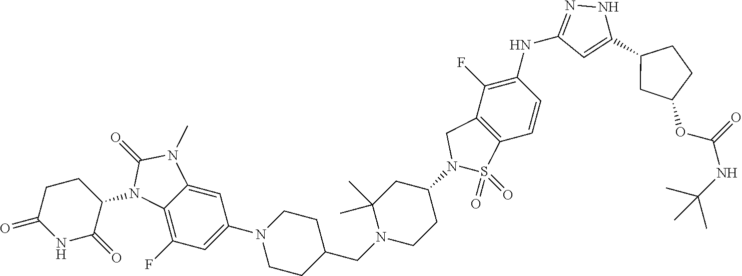 [SMILES: Cn1c(=O)n([C@H]2CCC(=O)NC2=O)c2c(F)cc(N3CCC(CN4CC[C@@H](N5Cc6c(ccc(Nc7cc([C@@H]8CC[C@H](OC(=O)NC(C)(C)C)C8)[nH]n7)c6F)S5(=O)=O)CC4(C)C)CC3)cc21]

The titled compound was synthesized in the procedures similar to Example 36 in a racemic form, which was further separated by chiral HPLC to give Example 49 (100% ee); Retention time: 6.696 min. ¹H NMR (500 MHz, DMSO) δ 11.98 (s, 1H), 11.07 (s, 1H), 8.77 (s, 1H), 8.30 (t, J=8.1 Hz, 1H), 7.51 (d, J=8.6 Hz, 1H), 6.76 (s, 1H), 6.68 (s, 1H), 6.51 (d, J=14.6 Hz, 1H), 5.83 (s, 1H), 5.41 (s, 1H), 4.96 (d, J=15.6 Hz, 1H), 4.52 (d, J=15.5 Hz, 2H), 3.69-3.65 (m, 3H), 3.11-2.93 (m, 2H), 2.85-2.75 (m, 2H), 2.71-2.66 (m, 2H), 2.65-2.60 (m, 4H), 2.45-2.41 (m, 1H), 2.31-2.17 (m, 1H), 2.14-1.84 (m, 7H), 1.81-1.63 (m, 6H), 1.62-1.46 (m, 2H), 1.31-1.23 (m, 1H), 1.21 (s, 9H), 1.10 (s, 3H), 0.98-0.96 (m, 3H). LC-MS (ESI): m/z [M+H]⁺=935.5;

Chiral analytical method: Column: ICHIRALPAK IF 4.6*150 mm 5 μm; Mobile phase: A for MtBE (0.1%2M NH3 MeOH) (%) and B for MeOH:DCM=50:50(%); Gradient: Mobile Phase A: Mobile Phase B=30:70 (v/v); HPLC Equipment: HPLC-Agilent, Back pressure: 100 bar; Column temperature: 35° C.

Chiral Prep-HPLC Condition: CHIRALPAK IF 250*20.0 mml, 5 μm; Mobile phase: A for MtBE (0.1%2M NH3 MeOH) (%) and B for MeOH:DCM=50:50; Gradient: Mobile Phase A: Mobile Phase B=30:70 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 50a and 50b: (1S,3R)-3-(3-((2-((4S)-1-((1-(1-(2,6-dioxopiperidin-3-yl)-4-fluoro-3-methyl-2-oxo-2,3-dihydro-1H-benzo[d]imidazol-5-yl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl tert-butylcarbamate and (1S,3R)-3-(3-((2-((4R)-1-((1-(1-(2,6-dioxopiperidin-3-yl)-4-fluoro-3-methyl-2-oxo-2,3-dihydro-1H-benzo[d]imidazol-5-yl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl tert-butylcarbamate

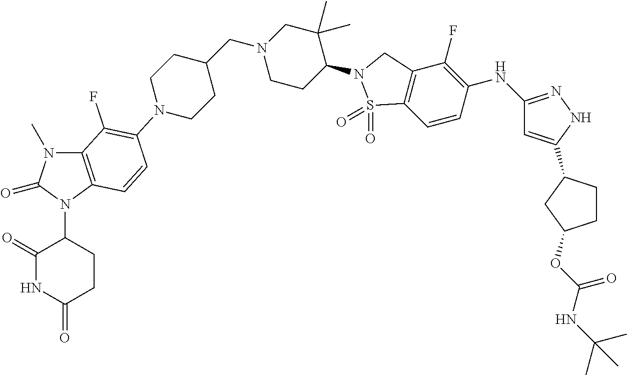 [SMILES: Cn1c(=O)n(C2CCC(=O)NC2=O)c2ccc(N3CCC(CN4CC[C@H](N5Cc6c(ccc(Nc7cc([C@@H]8CC[C@H](OC(=O)NC(C)(C)C)C8)[nH]n7)c6F)S5(=O)=O)C(C)(C)C4)CC3)c(F)c21]

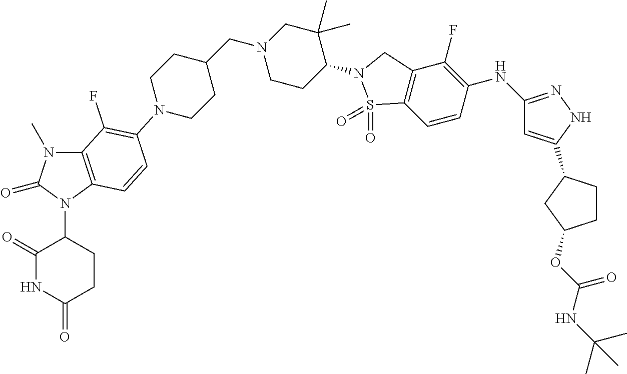 [SMILES: Cn1c(=O)n(C2CCC(=O)NC2=O)c2ccc(N3CCC(CN4CC[C@@H](N5Cc6c(ccc(Nc7cc([C@@H]8CC[C@H](OC(=O)NC(C)(C)C)C8)[nH]n7)c6F)S5(=O)=O)C(C)(C)C4)CC3)c(F)c21]

The titled compound was synthesized in the procedures similar to Example 36 in a racemic form, which was further separated by Chiral Prep-HPLC to give:

Enantiomer 1 (Example 50a, 100% ee); Retention time: 7.605 min. ¹H NMR (500 MHz, DMSO) δ 11.97 (s, 1H), 11.09 (s, 1H), 8.77 (s, 1H), 8.31 (s, 1H), 7.54 (d, J=8.6 Hz, 1H), 6.85 (d, J=8.4 Hz, 1H), 6.80-6.70 (m, 2H), 5.83 (s, 1H), 5.33 (dd, J=12.7, 5.1 Hz, 1H), 5.06-4.96 (m, 1H), 4.54 (s, 2H), 3.47 (s, 3H), 3.26-3.20 (m, 3H), 3.11-3.01 (m, 1H), 3.00-2.86 (m, 2H), 2.74-2.64 (m, 3H), 2.57-2.54 (m, 1H), 2.45-2.42 (m, 1H), 2.26-2.09 (m, 3H), 2.05-1.95 (m, 3H), 1.94-1.78 (m, 4H), 1.76-1.68 (m, 2H), 1.64-1.56 (m, 2H), 1.54-1.46 (m, 1H), 1.34-1.26 (m, 2H), 1.21 (s, 9H), 1.13 (s, 3H), 0.94 (s, 3H). LC-MS (ESI): m/z [M+H]⁺=935.4;

Enantiomer 1 (Example 50b, 100% ee); Retention time: 12.254, 17.556 min. ¹H NMR (500 MHz, DMSO) δ 11.97 (s, 1H), 11.09 (s, 1H), 8.77 (s, 1H), 8.31 (s, 1H), 7.54 (d, J=8.6 Hz, 1H), 6.85 (d, J=8.4 Hz, 1H), 6.80-6.70 (m, 2H), 5.83 (s, 1H), 5.33 (dd, J=12.7, 5.1 Hz, 1H), 5.06-4.96 (m, 1H), 4.54 (s, 2H), 3.47 (s, 3H), 3.26-3.20 (m, 3H), 3.11-3.01 (m, 1H), 3.00-2.86 (m, 2H), 2.74-2.64 (m, 3H), 2.57-2.54 (m, 1H), 2.45-2.42 (m, 1H), 2.26-2.09 (m, 3H), 2.05-1.95 (m, 3H), 1.94-1.78 (m, 4H), 1.76-1.68 (m, 2H), 1.64-1.56 (m, 2H), 1.54-1.46 (m, 1H), 1.34-1.26 (m, 2H), 1.21 (s, 9H), 1.13 (s, 3H), 0.94 (s, 3H). LC-MS (ESI): m/z [M+H]⁺=935.4;

Chiral analytical method: Column: ICHIRALPAK SA 4.6*150 mm 5 μm; Mobile phase: A for MtBE (0.1%2M NH3 MeOH) (%) and B for MeOH:DCM=50:50(%); Gradient: Mobile Phase A: Mobile Phase B=50:50 (v/v); HPLC Equipment: HPLC-Agilent, Back pressure: 100 bar; Column temperature: 35° C.

Chiral Prep-HPLC Condition: CHIRALPAK SA 250*20.0 mml, 5 μm; Mobile phase: A for MtBE (0.1%2M NH3 MeOH) (%) and B for MeOH:DCM=50:50; Gradient: Mobile Phase A: Mobile Phase B=50:50 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 51a and 51b: (1S,3R)-3-(3-((2-((4S)-1-((1-(1-(2,6-dioxopiperidin-3-yl)-3-ethyl-4-fluoro-2-oxo-2,3-dihydro-1H-benzo[d]imidazol-5-yl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl tert-butylcarbamate and (1S,3R)-3-(3-((2-((4R)-1-((1-(1-(2,6-dioxopiperidin-3-yl)-3-ethyl-4-fluoro-2-oxo-2,3-dihydro-1H-benzo[d]imidazol-5-yl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl tert-butylcarbamate

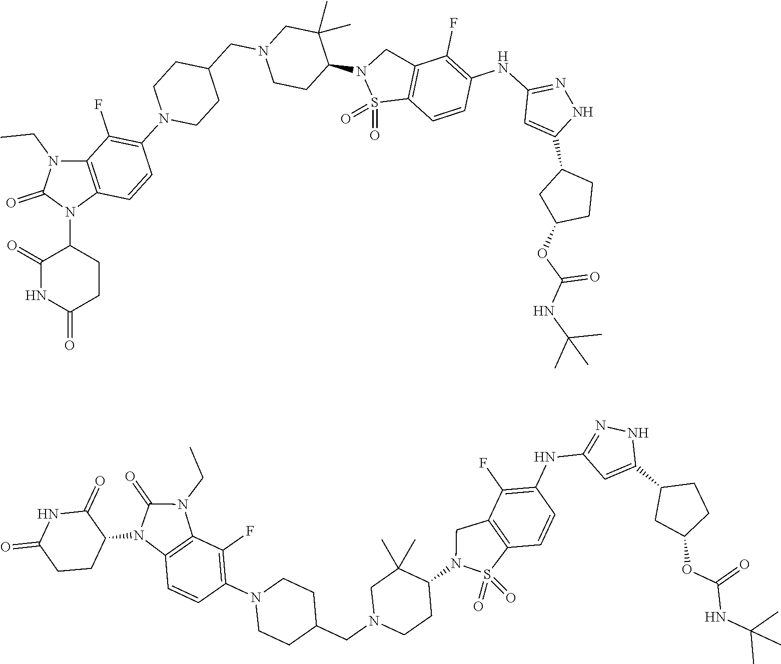 [SMILES: CCn1c(=O)n(C2CCC(=O)NC2=O)c2ccc(N3CCC(CN4CC[C@H](N5Cc6c(ccc(Nc7cc([C@@H]8CC[C@H](OC(=O)NC(C)(C)C)C8)[nH]n7)c6F)S5(=O)=O)C(C)(C)C4)CC3)c(F)c21 ; CCn1c(=O)n([C@@H]2CCC(=O)NC2=O)c2ccc(N3CCC(CN4CC[C@@H](N5Cc6c(ccc(Nc7cc([C@@H]8CC[C@H](OC(=O)NC(C)(C)C)C8)[nH]n7)c6F)S5(=O)=O)C(C)(C)C4)CC3)c(F)c21]

The titled compound was synthesized in the procedures similar to Example 36 in a racemic form, which was further separated by Chiral Prep-HPLC to give:

Enantiomer 1 (Example 51a, 100% ee); Retention time: 5.993 min. ¹H NMR (500 MHz, DMSO) δ 11.98 (s, 1H), 11.10 (s, 1H), 8.77 (s, 1H), 8.30 (d, J=7.6 Hz, 1H), 7.54 (d, J=8.6 Hz, 1H), 6.86 (d, J=8.4 Hz, 1H), 6.75 (t, J=7.9 Hz, 2H), 5.83 (s, 1H), 5.33 (dd, J=12.5, 5.3 Hz, 1H), 5.06-4.96 (m, 1H), 4.54 (s, 2H), 3.93 (d, J=7.6 Hz, 2H), 3.26-3.18 (m, 3H), 3.12-3.02 (m, 1H), 3.00-2.84 (m, 2H), 2.76-2.66 (m, 3H), 2.46-2.42 (m, 1H), 2.22-2.10 (m, 3H), 2.04-1.96 (m, 3H), 1.94-1.78 (m, 5H), 1.76-1.68 (m, 2H), 1.66-1.55 (m, 2H), 1.53-1.47 (m, 1H), 1.34-1.22 (m, 6H), 1.21 (s, 9H), 1.13 (s, 3H), 0.94 (s, 3H). LC-MS (ESI): m/z [M+H]⁺=949.4;

Enantiomer 1 (Example 51b, 100% ee); Retention time: 9.182 min. ¹H NMR (500 MHz, DMSO) δ 11.98 (s, 1H), 11.10 (s, 1H), 8.77 (s, 1H), 8.30 (d, J=7.6 Hz, 1H), 7.54 (d, J=8.6 Hz, 1H), 6.86 (d, J=8.4 Hz, 1H), 6.75 (t, J=7.9 Hz, 2H), 5.83 (s, 1H), 5.33 (dd, J=12.5, 5.3 Hz, 1H), 5.06-4.96 (m, 1H), 4.54 (s, 2H), 3.93 (d, J=7.6 Hz, 2H), 3.26-3.18 (m, 3H), 3.12-3.02 (m, 1H), 3.00-2.84 (m, 2H), 2.76-2.66 (m, 3H), 2.46-2.42 (m, 1H), 2.22-2.10 (m, 3H), 2.04-1.96 (m, 3H), 1.94-1.78 (m, 5H), 1.76-1.68 (m, 2H), 1.66-1.55 (m, 2H), 1.53-1.47 (m, 1H), 1.34-1.22 (m, 6H), 1.21 (s, 9H), 1.13 (s, 3H), 0.94 (s, 3H). LC-MS (ESI): m/z [M+H]⁺=949.4;

Chiral analytical method: Column: ICHIRALPAK IE 4.6*150 mm 5 μm; Mobile phase: A for MtBE (0.1%2M NH3 MeOH) (%) and B for MeOH:DCM=50:50(%); Gradient: Mobile Phase A: Mobile Phase B=20:80 (v/v); HPLC Equipment: HPLC-Agilent, Back pressure: 100 bar; Column temperature: 35° C.

Chiral Prep-HPLC Condition: CHIRALPAK IE 250*20.0 mml, 5 μm; Mobile phase: A for MtBE (0.1%2M NH3 MeOH) (%) and B for MeOH:DCM=50:50; Gradient: Mobile Phase A: Mobile Phase B=20:80 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 52: (1S,3R)-3-(3-((2-((4R)-1-((1-(1-(2,6-dioxopiperidin-3-yl)-3-methyl-2-oxo-2,3-dihydro-1H-benzo[d]imidazol-5-yl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl tert-butylcarbamate

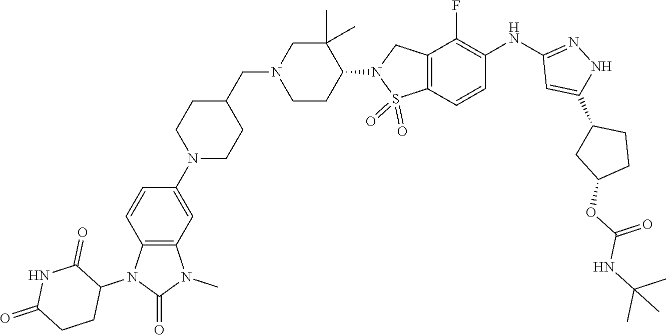 [SMILES: Cn1c(=O)n(C2CCC(=O)NC2=O)c2ccc(N3CCC(CN4CC[C@@H](N5Cc6c(ccc(Nc7cc([C@@H]8CC[C@H](OC(=O)NC(C)(C)C)C8)[nH]n7)c6F)S5(=O)=O)C(C)(C)C4)CC3)cc21]

### Step 1: tert-butyl (R)-4-(5-bromo-4-fluoro-1,1-dioxidobenzo[d]isothiazol-2(3H)-yl)-3,3-dimethylpiperidine-1-carboxylate

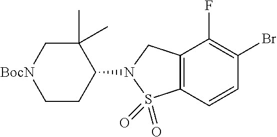 [SMILES: CC(C)(C)OC(=O)N1CC[C@@H](N2Cc3c(ccc(Br)c3F)S2(=O)=O)C(C)(C)C1]

The titled compound was synthesised in the procedure similar to example 16 and further separated by chiral HPLC to give Enantiomer 2 (100% ee); Retention time: 0.927 min. LC-MS (ESI): m/z [M+H]⁺=477.4

Chiral analytical method: Column: CHIRAL ART Cellulose SB 4.6*50 mm 3 m; Mobile phase: A for CO2 and B for MeOH; Gradient (B %) 5% to 20% in 2 min, hold 1 min at 20%; HPLC Equipment: HPLC-Agilent, Back pressure: 100 bar; Column temperature: 35° C.

Chiral Prep-HPLC Condition: CHIRAL ART Cellulose SB 250*50.0 mml, 10 μm; Mobile phase: A for CO2 and B for MeOH; Gradient (B %) 5% to 20% in 2 min, hold 1 min at 20%; Flow Rate: 180 mL/min, Wavelength: UV 220 nm, Prep-HPLC Equipment: Prep-SFC-350; Column temperature: 25° C.

### Step 2: (1S,3R)-3-(3-((2-((4R)-1-((1-(1-(2,6-dioxopiperidin-3-yl)-3-methyl-2-oxo-2,3-dihydro-1H-benzo[d]imidazol-5-yl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl tert-butylcarbamate

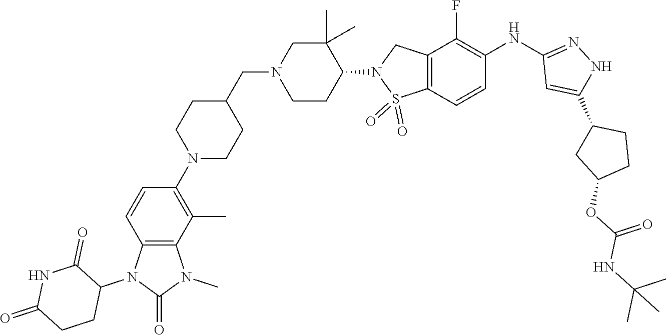 [SMILES: Cc1c(N2CCC(CN3CC[C@@H](N4Cc5c(ccc(Nc6cc([C@@H]7CC[C@H](OC(=O)NC(C)(C)C)C7)[nH]n6)c5F)S4(=O)=O)C(C)(C)C3)CC2)ccc2c1n(C)c(=O)n2C1CCC(=O)NC1=O]

The titled compound was synthesized in the procedures similar to Example 36. LC-MS (ESI): m/z [M+H]⁺=917.25; ¹H NMR (500 MHz, DMSO) δ=11.98 (s, 1H), 11.06 (s, 1H), 8.77 (s, 1H), 8.31 (s, 1H), 7.54 (d, J=8.6, 1H), 6.92 (d, J=8.6, 1H), 6.83 (s, 1H), 6.76 (s, 1H), 6.64 (d, J=9.1, 1H), 5.84 (s, 1H), 5.28 (d, J=13.1, 1H), 4.98 (s, 1H), 4.55 (s, 2H), 3.60 (m, 4H), 3.06 (m, 1H), 2.95 (m, 2H), 2.89 (m, 1H), 2.66 (m, 2H), 2.59 (m, 4H), 2.18 (m, 2H), 2.13-2.10 (m, 1H), 1.99 (m, 4H), 1.91 (m, 1H), 1.83 (m, 1H), 1.80 (m, 2H), 1.71 (m, 2H), 1.61 (m, 2H), 1.53-1.49 (m, 1H), 1.21 (m, 11H), 1.13 (s, 3H), 0.94 (s, 3H).

### Example 53: (1S,3R)-3-(3-((2-((4R)-1-((1-(1-(2,6-dioxopiperidin-3-yl)-3-methyl-2-oxo-2,3-dihydro-1H-imidazo[4,5-b]pyridin-5-yl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl tert-butylcarbamate

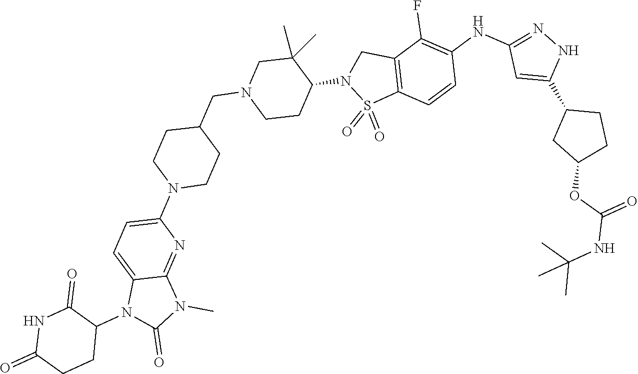 [SMILES: Cn1c(=O)n(C2CCC(=O)NC2=O)c2ccc(N3CCC(CN4CC[C@@H](N5Cc6c(ccc(Nc7cc([C@@H]8CC[C@H](OC(=O)NC(C)(C)C)C8)[nH]n7)c6F)S5(=O)=O)C(C)(C)C4)CC3)nc21]

### Step 1: 5-(4-(dimethoxymethyl)piperidin-1-yl)-3-methyl-1,3-dihydro-2H-imidazo[4,5-b]pyridin-2-one

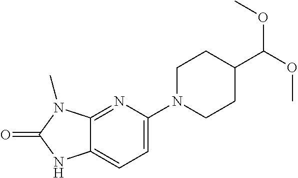 [SMILES: COC(OC)C1CCN(c2ccc3[nH]c(=O)n(C)c3n2)CC1]

To a mixture of 5-chloro-3-methyl-1,3-dihydro-2H-imidazo[4,5-b]pyridin-2-one (500 mg, 2.72 mmol) and 4-(dimethoxymethyl)piperidine (650 mg, 4.09 mmol) in andydrous THF (15 mL) was added Pd₂(dba)₃ (500 mg, 0.54 mmol), RuPhos (508 mg, 1 mmol) and LiHMDS (10.9 mL, 10.9 mmol, 1 M in THF) at 0° C. The reaction mixture was stirred under nitrogen atmosphere at 70° C. for 1 h. The mixture was diluted with EA (100 mL), washed with NH₄Cl aq. (2×50 mL) and brine (50 mL). The organic layer was dried over anhydrous Na₂SO₄, filtered and concentrated under reduced pressure. The residue was purified by silica gel column chromatography, eluting with DCM/MeOH (1:30) to afford the product (420 mg, 50% yield). LC-MS (ESI): m/z [M+H]⁺=307.3.

### Step 2: 3-(5-(4-(dimethoxymethyl)piperidin-1-yl)-3-methyl-2-oxo-2,3-dihydro-1H-imidazo[4,5-b]pyridin-1-yl)piperidine-2,6-dione

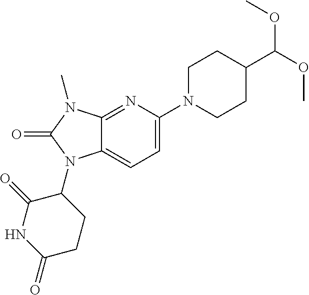 [SMILES: COC(OC)C1CCN(c2ccc3c(n2)n(C)c(=O)n3C2CCC(=O)NC2=O)CC1]

To a stirring solution of 5-(4-(dimethoxymethyl)piperidin-1-yl)-3-methyl-1,3-dihydro-2H-imidazo[4,5-b]pyridin-2-one (420 mg, 1.37 mmol) and 3-bromopiperidine-2,6-dione (526 mg, 2.74 mmol) in THF (10 mL) was added NaH (220 mg, 5.48 mmol, 60% in mineral oil) slowly at 0° C. The resulting solution was heated to 70° C. for 2 h. The solution was cooled to 0° C., quenched with NH₄Cl aq. (50 mL). The mixture was extracted with DCM (100 mL×3). The combined organic layers were washed with brine (50 mL) and dried over anhydrous Na₂SO₄. After filtration, the filtrate was concentrated under reduced pressure. The residue was purified by silica gel column chromatography, eluting with DCM/MeOH (1:30) to afford the product (390 mg, 68% yield). LC-MS (ESI): m/z [M+H]⁺=418.4.

### Step 3: 1-(1-(2,6-dioxopiperidin-3-yl)-3-methyl-2-oxo-2,3-dihydro-1H-imidazo[4,5-b]pyridin-5-yl)piperidine-4-carbaldehyde

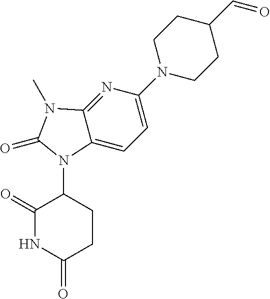 [SMILES: Cn1c(=O)n(C2CCC(=O)NC2=O)c2ccc(N3CCC(C=O)CC3)nc21]

To a mixture of 3-(5-(4-(dimethoxymethyl)piperidin-1-yl)-3-methyl-2-oxo-2,3-dihydro-1H-imidazo[4,5-b]pyridin-1-yl)piperidine-2,6-dione (390 mg, 0.93 mmol) in HCl aq (10 mL, 8 N) was stirred at room temperature for 3 h. The solution was cooled to 0° C., quenched with NaHCO₃ aq. (150 mL). The mixture was extracted with DCM (100 mL×3). The combined organic layers were washed with brine (50 mL) and dried over anhydrous Na₂SO₄. After filtration, the filtrate was concentrated under reduced pressure to afford the product (310 mg, 89% yield). LC-MS (ESI): m/z [M+H]⁺=372.3.

### Step 4: (1S,3R)-3-(3-((2-((4R)-1-((1-(1-(2,6-dioxopiperidin-3-yl)-3-methyl-2-oxo-2,3-dihydro-1H-imidazo[4,5-b]pyridin-5-yl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl tert-butylcarbamate

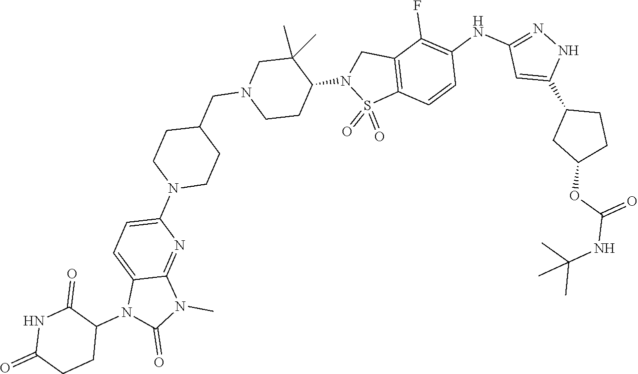 [SMILES: Cn1c(=O)n(C2CCC(=O)NC2=O)c2ccc(N3CCC(CN4CC[C@@H](N5Cc6c(ccc(Nc7cc([C@@H]8CC[C@H](OC(=O)NC(C)(C)C)C8)[nH]n7)c6F)S5(=O)=O)C(C)(C)C4)CC3)nc21]

The titled compound was synthesized in the procedures similar to Example 36. ¹H NMR (500 MHz, DMSO) δ 11.99 (s, 1H), 11.07 (s, 1H), 8.76 (s, 1H), 8.30 (s, 1H), 7.54 (d, J=8.7 Hz, 1H), 7.29 (d, J=8.5 Hz, 1H), 6.94 (s, 1H), 6.44 (d, J=8.3 Hz, 1H), 5.83 (s, 1H), 5.30 (d, J=12.5 Hz, 1H), 5.00 (s, 1H), 4.54 (s, 2H), 4.2-4.18 (m, 2H), 3.57 (s, 1H), 3.29-3.28 (m, 3H), 3.24-3.21 (m, 1H), 3.06 (s, 1H), 2.95-2.91 (m, 3H), 2.75 (s, 2H), 2.67-2.64 (m, 5H), 2.36 (s, 1H), 2.23-2.05 (m, 4H), 1.97-1.91 (m, 5H), 1.84-1.67 (m, 5H), 1.24-1.09 (m, 5H), 1.04-1.02 (m, 6H), 0.95-0.92 (m, 3H). LC-MS (ESI): m/z [M+H]⁺=918.65.

### Example 54: (1S,3R)-3-(3-((2-((4R)-1-((1-(1-(2,6-dioxopiperidin-3-yl)-3,4-dimethyl-2-oxo-2,3-dihydro-1H-benzo[d]imidazol-5-yl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl tert-butylcarbamate

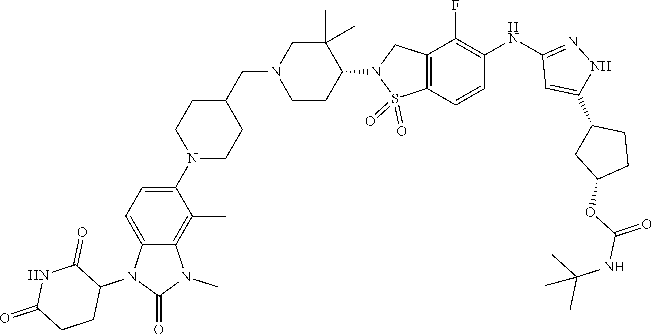 [SMILES: Cc1c(N2CCC(CN3CC[C@@H](N4Cc5c(ccc(Nc6cc([C@@H]7CC[C@H](OC(=O)NC(C)(C)C)C7)[nH]n6)c5F)S4(=O)=O)C(C)(C)C3)CC2)ccc2c1n(C)c(=O)n2C1CCC(=O)NC1=O]

The titled compound was synthesized in the procedures similar to Example 42. ¹H NMR (500 MHz, DMSO) δ 11.98 (s, 1H), 11.06 (s, 1H), 8.77 (d, J=2.0 Hz, 1H), 8.29 (s, 1H), 7.54 (d, J=8.6 Hz, 1H), 6.86 (d, J=8.4 Hz, 1H), 6.80-6.59 (m, 2H), 5.83 (s, 1H), 5.32-5.29 (m, 1H), 4.98 (s, 1H), 4.65-4.49 (m, 2H), 3.58 (s, 3H), 3.24-3.21 (m, 1H), 3.13-3.02 (m, 1H), 3.00-2.81 (m, 4H), 2.74-2.56 (m, 4H), 2.54-2.52 (m, 4H), 2.48-2.44 (m, 1H), 2.23-2.12 (m, 3H), 2.05-1.93 (m, 3H), 1.93-1.87 (m, 1H), 1.83-1.81 (m, 3H), 1.73-1.68 (m, 2H), 1.58 (s, 2H), 1.52-1.50 (m, 1H), 1.37-1.25 (m, 2H), 1.21 (s, 9H), 1.13 (s, 3H), 0.94 (s, 3H). LC-MS (ESI): m/z [M+H]⁺=931.45.

### Example 55: (1S,3R)-3-(3-((2-((S)-1-((1-(1-((S)-2,6-dioxopiperidin-3-yl)-4,7-difluoro-3-methyl-2-oxo-2,3-dihydro-1H-benzo[d]imidazol-5-yl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl tert-butylcarbamate

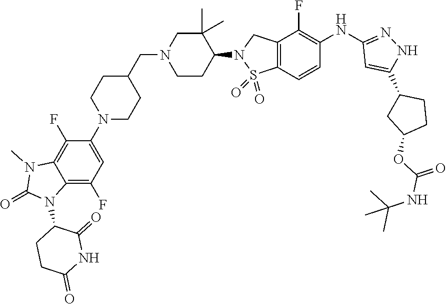 [SMILES: Cn1c(=O)n([C@H]2CCC(=O)NC2=O)c2c(F)cc(N3CCC(CN4CC[C@H](N5Cc6c(ccc(Nc7cc([C@@H]8CC[C@H](OC(=O)NC(C)(C)C)C8)[nH]n7)c6F)S5(=O)=O)C(C)(C)C4)CC3)c(F)c21]

### Step 1: 4-bromo-2,3,6-trifluoroaniline

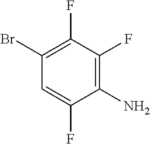 [SMILES: Nc1c(F)cc(Br)c(F)c1F]

To a solution of 2,3,6-trifluoroaniline (12.5 g, 85 mmol) in DMF (125 mL) at 0° C. was added NBS (16 g, 93.5 mmol). The reaction mixture was stirred for 2 h at rt. The resulting mixture was extracted with EA. The organic layers were washed with brine, The reaction was concentrated under vacuum. The crude was purified by combine-flash EA:PE=100:0 to 15:85 to give the product (13.5 g, 70%) as off-white solid. LCMS (M+H) m/z: 226.

### Step 2: 1-bromo-2,3,5-trifluoro-4-nitrobenzene

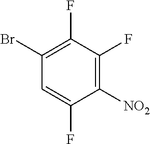 [SMILES: O=[N+]([O-])c1c(F)cc(Br)c(F)c1F]

To a solution of 4-bromo-2,3,6-trifluoroaniline (13.5 g, 59 mmol) in AcOH (47.25 mL) was added NaBO₃ (24.4 g, 295 mmol) in AcOH (303.75 mL) at 55° C. After stirring of the reaction mixture for 2 h at 55° C. To the above mixture was added NaBO₃ (24.4 g, 5 eq) in portions at 55° C. The resulting mixture was stirred for 1.5 h at 55° C., The resulting mixture was extracted with EA (3×20 mL). After filtration, the filtrate was concentrated under reduced pressure. The crude was purified by combine-flash PE:EA=100:0 to 90:10. The product (9 g, 82%) as off-white solid. LCMS (M+H) m/z: 256.

### Step 3: 3-bromo-2,5-difluoro-N-methyl-6-nitroaniline

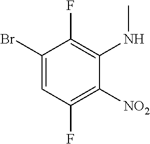

To a solution of 1-bromo-2,3,5-trifluoro-4-nitrobenzene (9 g, 35 mmol) in DMF (100 ml) was added NH₂CH₃—HCl (9 g, 31.7 mmol), K₂CO₃ (10 g, 70 mmol), The reaction mixture for 5 h at rt The resulting mixture was extracted with EA The organic layers was washed with brine. After filtration, the filtrate was concentrated under reduced pressure. The crude was purified by combine-flash PE:EA=100:0 to 80:20. The product (8 g, 82%) as off-white solid. LCMS (M+H) m/z: 267.

### Step 4: 5-bromo-3,6-difluoro-N1-methylbenzene-1,2-diamine

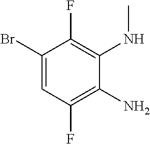

To a solution of 3-bromo-2,5-difluoro-N-methyl-6-nitroaniline (8 g, 30 mmol) in MeOH:H₂O=4:1 (300 mL) was added Fe (5 g, 90 eq) and NH₄Cl (7.95 g, 150 mmol). The resulting solution was stirred for 3 h at 80° C. The resulting solution was extracted with EA. The organic layers were washed with brine. dried over anhydrous Na₂SO₄ The resulting mixture was concentrated under vacuum. The residue was purified by combi-flash (EtOAc/PE=46%) to give the product (6 g, 67.2%) as red oil. LCMS (M+H) m/z: 237.

### Step 5: 6-bromo-4,7-difluoro-1-methyl-1,3-dihydro-2H-benzo[d]imidazol-2-one

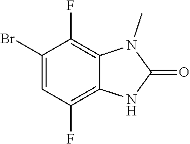

To a solution of 5-bromo-3,6-difluoro-N1-methylbenzene-1,2-diamine (6 g, 25.3 mmol) in MeCN (100 mL) were added CDI (2.6 g, 37.95 mmol). The resulting solution was stirred for 3 h at 70° C. The resulting mixture was concentrated under vacuum. The residue was purified by combi-flash (EtOAc/PE=40%) to give the product (5 g, 75%) as red solid.

LCMS (M+H) m/z: 263.

### Step 6: 3-(5-bromo-4,7-difluoro-3-methyl-2-oxo-2,3-dihydro-1H-benzo[d]imidazole-1-yl)piperidine-2,6-dione

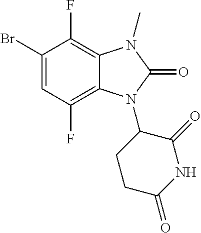 [SMILES: Cn1c(=O)n(C2CCC(=O)NC2=O)c2c(F)cc(Br)c(F)c21]

To a solution of 6-bromo-4,7-difluoro-1-methyl-1,3-dihydro-2H-benzo[d]imidazol-2-one (2 g, 7.6 mmol) in THF (50 mL) were added sodium hydride (60%)(608 mg, 15.2 mmol) and 3-bromopiperidine-2,6-dione (2.92 g, 15.2 mmol). The resulting solution was stirred for 12 h at 60° C. The resulting mixture was concentrated under vacuum. The residue was purified by combi-flash (EtOAc/PE=40%) to give the product (550 mg, 20%) as white solid. LCMS (M+H) m/z: 374.

### Step 7: 3-(5-(4-(dimethoxymethyl)piperidin-1-yl)-4,7-difluoro-3-methyl-2-oxo-2,3-dihydro-1H-benzo[d]imidazol-1-yl)piperidine-2,6-dione

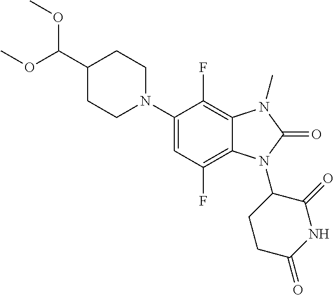 [SMILES: COC(OC)C1CCN(c2cc(F)c3c(c2F)n(C)c(=O)n3C2CCC(=O)NC2=O)CC1]

To a solution of 3-(5-bromo-4,7-difluoro-3-methyl-2-oxo-2,3-dihydro-1H-benzo[d]imidazol-1-yl)piperidine-2,6-dione (540 mg, 1.4 mmol) in dioxane (10 mL) were added 4-(dimethoxymethyl)piperidine (329 mg, 2.1 mmol), Pd-PEPPSI-IPentCl 2-methylpyridine (58.8 mg, 0.07 mmol) and Cs₂CO₃ (682.5 mg, 2.1 mmol). The resulting solution was stirred for 12 h at 100° C. under nitrogen atmosphere. The resulting mixture was concentrated under vacuum. The residue was purified by combi-flash (EtOAc/PE=56%) to give the product (213.0 mg, 31%) as white solid. LCMS (M+H) m/z: 453.10.

### Step 8: 1-(1-(2,6-dioxopiperidin-3-yl)-4,7-difluoro-3-methyl-2-oxo-2,3-dihydro-1H-benzo[d]imidazol-5-yl)piperidine-4-carbaldehyde

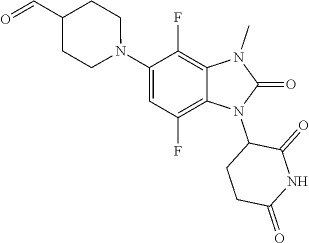 [SMILES: Cn1c(=O)n(C2CCC(=O)NC2=O)c2c(F)cc(N3CCC(C=O)CC3)c(F)c21]

3-(5-(4-(dimethoxymethyl)piperidin-1-yl)-4,7-difluoro-3-methyl-2-oxo-2,3-dihydro-1H-benzo[d]imidazol-1-yl)piperidine-2,6-dione (251 mg, 0.5 mmol) was dissolved in formic acid (5 mL), stirred for 2 hours at room temperature. The resulting mixture was concentrated under reduce pressure, dried over in vacuum, to afford the product (173 mg, 85%). LC-MS (ESI): m/z [M+H]⁺=407.4.

### Step 9: tert-butyl (S)-4-(5-bromo-4-fluoro-1,1-dioxidobenzo[d]isothiazol-2(3H)-yl)-3,3-dimethylpiperidine-1-carboxylate

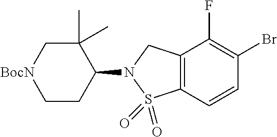 [SMILES: CC(C)(C)OC(=O)N1CC[C@H](N2Cc3c(ccc(Br)c3F)S2(=O)=O)C(C)(C)C1]

The titled compound was synthesised in the procedure similar to example 16 and further separated by chiral HPLC to give Enantiomer 1 (100% ee); Retention time: 0.812 min. LC-MS (ESI): m/z [M+H]⁺=477.4

Chiral analytical method: Column: CHIRAL ART Cellulose SB 4.6*50 mm 3 m; Mobile phase: A for CO2 and B for MeOH; Gradient (B %) 5% to 20% in 2 min, hold 1 min at 20%; HPLC Equipment: HPLC-Agilent, Back pressure: 100 bar; Column temperature: 35° C.

Chiral Prep-HPLC Condition: CHIRAL ART Cellulose SB 250*50.0 mml, 10 μm; Mobile phase: A for CO2 and B for MeOH; Gradient (B %) 5% to 20% in 2 min, hold 1 min at 20%; Flow Rate: 180 mL/min, Wavelength: UV 220 nm, Prep-HPLC Equipment: Prep-SFC-350; Column temperature: 25° C.

### Step 10: (1S,3R)-3-(3-((2-((S)-1-((1-(1-((S)-2,6-dioxopiperidin-3-yl)-4,7-difluoro-3-methyl-2-oxo-2,3-dihydro-1H-benzo[d]imidazol-5-yl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl tert-butylcarbamate

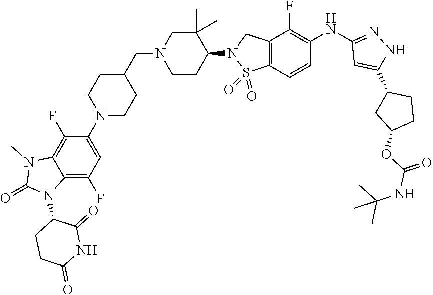 [SMILES: Cn1c(=O)n([C@H]2CCC(=O)NC2=O)c2c(F)cc(N3CCC(CN4CC[C@H](N5Cc6c(ccc(Nc7cc([C@@H]8CC[C@H](OC(=O)NC(C)(C)C)C8)[nH]n7)c6F)S5(=O)=O)C(C)(C)C4)CC3)c(F)c21]

The titled compound was synthesized in the procedures similar to Example 36 in a racemic form, which was further separated by Chiral Prep-HPLC to give Example 55; Retention time: 5.97 min. ¹H NMR (500 MHz, DMSO) δ 11.98 (s, 1H), 11.11 (s, 1H), 8.77 (s, 1H), 8.31 (t, J=8.1 Hz, 1H), 7.54 (d, J=8.6 Hz, 1H), 6.71 (d, J=48.5 Hz, 2H), 5.83 (s, 1H), 5.47 (s, 1H), 4.97 (s, 1H), 4.54 (s, 2H), 3.50 (s, 3H), 3.28-3.20 (m, 3H), 3.10-3.02 (m, 1H), 2.96 (s, 2H), 2.67-2.60 (m, 8H), 2.23-2.08 (m, 3H), 2.01-1.96 (m, 3H), 1.90 (s, 1H), 1.82-1.80 (m, 2H), 1.73-1.71 (m, 1H), 1.61 (s, 2H), 1.52 (s, 1H), 1.32-1.23 (m, 2H), 1.21 (s, 9H), 1.12 (s, 3H), 0.94 (s, 3H). LC-MS (ESI): m/z [M+H]⁺=953.27.

Chiral analytical method: Column: I-Cellulose-5, 4.6 mm×250 mm, 5 um; Mobile phase: A for Hexane and B for EtOH (0.1% 2M NH₃ MeOH); Gradient: Mobile Phase A: Mobile Phase B=30:70 (v/v); HPLC Equipment: HPLC-Agilent, Back pressure: 100 bar; Column temperature: 35° C.

Chiral Prep-HPLC Condition: Column: I-Cellulose-5, 21.2 mm×250 mm, 5 um; Mobile phase: A for Hexane and B for EtOH (0.2% 2M NH₃ MeOH); Gradient: Mobile Phase A: Mobile Phase B=30:70 (v/v); Flow Rate: 20 mL/min, Wave Length: UV 200 nm and 270 nm, Prep-HPLC Equipment: Prep-HPLC-Gilson, Back pressure: 100 bar; Column temperature: 25° C.

### Example 56: (1S,3R)-3-(3-((2-((R)-1-(((R)-1-(1-((S)-2,6-dioxopiperidin-3-yl)-4-fluoro-3-methyl-2-oxo-2,3-dihydro-1H-benzo[d]imidazol-5-yl)-3,3-dimethylpiperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl tert-butylcarbamate

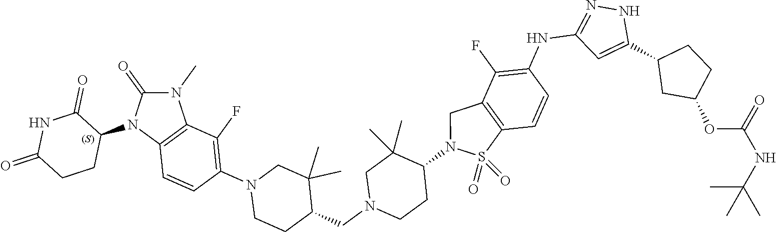

The racemic compound was synthesized in the procedures similar to Example 36, which was further separated by Chiral Prep-HPLC to afford the desired product (99.2% ee); Retention time: 7.96 min. ¹H NMR (500 MHz, DMSO-d₆) δ 11.98 (s, 1H), 11.10 (s, 1H), 8.77 (s, 1H), 8.31 (t, J=5 Hz, 1H), 7.54 (d, J=10 Hz, 1H), 6.86-6.81 (m, 1H), 6.79-6.74 (m, 1H), 6.73-6.67 (m, 1H), 5.83 (s, 1H), 5.37-5.29 (m, 1H), 5.03-4.95 (m, 1H), 4.45 (s, 2H), 3.47 (s, 3H), 3.26-3.16 (m, 2H), 3.13-3.00 (m, 2H), 2.94-2.78 (m, 2H), 2.74-2.58 (m, 4H), 2.48-2.42 (m, 1H), 2.41-2.29 (m, 2H), 2.20-2.09 (m, 1H), 2.06-1.94 (m, 4H), 1.93-1.79 (m, 3H), 1.78-1.66 (m, 2H), 1.63-1.32 (m, 5H), 1.29-1.17 (m, 9H), 1.12 (s, 3H), 1.03-0.89 (m, 9H). LC-MS (ESI): m/z [M+H]⁺=963.

Chiral analytical method: Amylose-SA, 4.6 mm×250 mm, 5 um; Mobile phase: A for MtBE (0.1% 2M NH3 MeOH) and B for DCM:MeOH (50:50); Gradient: Mobile Phase A: Mobile Phase B=40:60 (v/v); HPLC Equipment: HPLC-Agilent, Back pressure: 100 bar; Column temperature: 35° C.

Chiral Prep-HPLC Condition: Column: Amylose-SA, 250 mm×20 mm, 5 um; Mobile phase: A for MtBE (0.1% 2M NH3 MeOH) and B for DCM:MeOH (50:50); Gradient: Mobile Phase A: Mobile Phase B=40:60 (v/v); Flow Rate: 20 mL/min, Wave Length: UV 200 nm and 270 nm, Prep-HPLC Equipment: Prep-HPLC-Gilson, Back pressure: 100 bar; Column temperature: 25° C.

### Example 57a and 57b: (1S,3R)-3-(3-((2-((4S)-1-((1-(1-(2,6-dioxopiperidin-3-yl)-4-fluoro-3-methyl-2-oxo-2,3-dihydro-1H-benzo[d]imidazol-5-yl)piperidin-4-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl tert-butylcarbamate and (1S,3R)-3-(3-((2-((4R)-1-((1-(1-(2,6-dioxopiperidin-3-yl)-4-fluoro-3-methyl-2-oxo-2,3-dihydro-1H-benzo[d]imidazol-5-yl)piperidin-4-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl tert-butylcarbamate

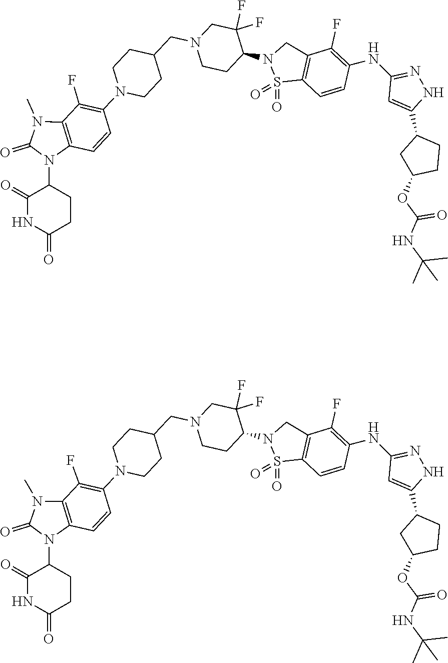 [SMILES: Cn1c(=O)n(C2CCC(=O)NC2=O)c2ccc(N3CCC(CN4CC[C@H](N5Cc6c(ccc(Nc7cc([C@@H]8CC[C@H](OC(=O)NC(C)(C)C)C8)[nH]n7)c6F)S5(=O)=O)C(F)(F)C4)CC3)c(F)c21 ; Cn1c(=O)n(C2CCC(=O)NC2=O)c2ccc(N3CCC(CN4CC[C@@H](N5Cc6c(ccc(Nc7cc([C@@H]8CC[C@H](OC(=O)NC(C)(C)C)C8)[nH]n7)c6F)S5(=O)=O)C(F)(F)C4)CC3)c(F)c21]

The titled compound was synthesized in the procedures similar to Example 36, which was further separated by Chiral Prep-HPLC to afford:

Enantiomer 1 (Example 57a, 100% ee); Retention time: 6.794, 7.269 min. ¹H NMR (500 MHz, DMSO) δ 11.99 (s, 1H), 11.10 (s, 1H), 8.83 (s, 1H), 8.33 (d, J=7.2 Hz, 1H), 7.58 (d, J=8.7 Hz, 1H), 6.85 (d, J=8.5 Hz, 1H), 6.83-6.66 (m, 2H), 5.84 (s, 1H), 5.33 (dd, J=12.5, 5.2 Hz, 1H), 5.06-4.96 (m, 1H), 4.75 (d, J=14.2 Hz, 1H), 4.54 (d, J=13.3 Hz, 1H), 4.07-3.94 (m, 1H), 3.47 (s, 3H), 3.27-3.14 (m, 4H), 3.11-3.01 (m, 1H), 3.00-2.84 (m, 2H), 2.73-2.65 (m, 3H), 2.45-2.42 (m, 1H), 2.35-2.28 (m, 3H), 2.26-2.14 (m, 2H), 2.07-1.96 (m, 2H), 1.94-1.86 (m, 1H), 1.84-1.77 (m, 3H), 1.75-1.55 (m, 4H), 1.35-1.25 (m, 2H), 1.21 (s, 9H). LC-MS (ESI): m/z [M+H]⁺=943.4;

Enantiomer 1 (Example 57b, 100% ee); Retention time: 11.267, 13.571 min. ¹H NMR (500 MHz, DMSO) δ 11.99 (s, 1H), 11.10 (s, 1H), 8.83 (s, 1H), 8.33 (d, J=7.2 Hz, 1H), 7.58 (d, J=8.7 Hz, 1H), 6.85 (d, J=8.5 Hz, 1H), 6.83-6.66 (m, 2H), 5.84 (s, 1H), 5.33 (dd, J=12.5, 5.2 Hz, 1H), 5.06-4.96 (m, 1H), 4.75 (d, J=14.2 Hz, 1H), 4.54 (d, J=13.3 Hz, 1H), 4.07-3.94 (m, 1H), 3.47 (s, 3H), 3.27-3.14 (m, 4H), 3.11-3.01 (m, 1H), 3.00-2.84 (m, 2H), 2.73-2.65 (m, 3H), 2.45-2.42 (m, 1H), 2.35-2.28 (m, 3H), 2.26-2.14 (m, 2H), 2.07-1.96 (m, 2H), 1.94-1.86 (m, 1H), 1.84-1.77 (m, 3H), 1.75-1.55 (m, 4H), 1.35-1.25 (m, 2H), 1.21 (s, 9H). LC-MS (ESI): m/z [M+H]⁺=943.4;

Chiral analytical method: Column: ICHIRALPAK IE 4.6*150 mm 5 μm; Mobile phase: A for MtBE (0.1%2M NH3 MeOH) (%) and B for MeOH:DCM=50:50(%); Gradient: Mobile Phase A: Mobile Phase B=20:80 (v/v); HPLC Equipment: HPLC-Agilent, Back pressure: 100 bar; Column temperature: 35° C.

Chiral Prep-HPLC Condition: CHIRALPAK IE 250*20.0 mml, 5 μm; Mobile phase: A for MtBE (0.1%2M NH3 MeOH) (%) and B for MeOH:DCM=50:50; Gradient: Mobile Phase A: Mobile Phase B=20:80 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 58a and 58b: (1S,3R)-3-(3-((2-((4S)-1′-(1-(2,6-dioxopiperidin-3-yl)-4-fluoro-3-methyl-2-oxo-2,3-dihydro-1H-benzo[d]imidazol-5-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl tert-butylcarbamate and (1S,3R)-3-(3-((2-((4R)-1′-(1-(2,6-dioxopiperidin-3-yl)-4-fluoro-3-methyl-2-oxo-2,3-dihydro-1H-benzo[d]imidazol-5-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl tert-butylcarbamate

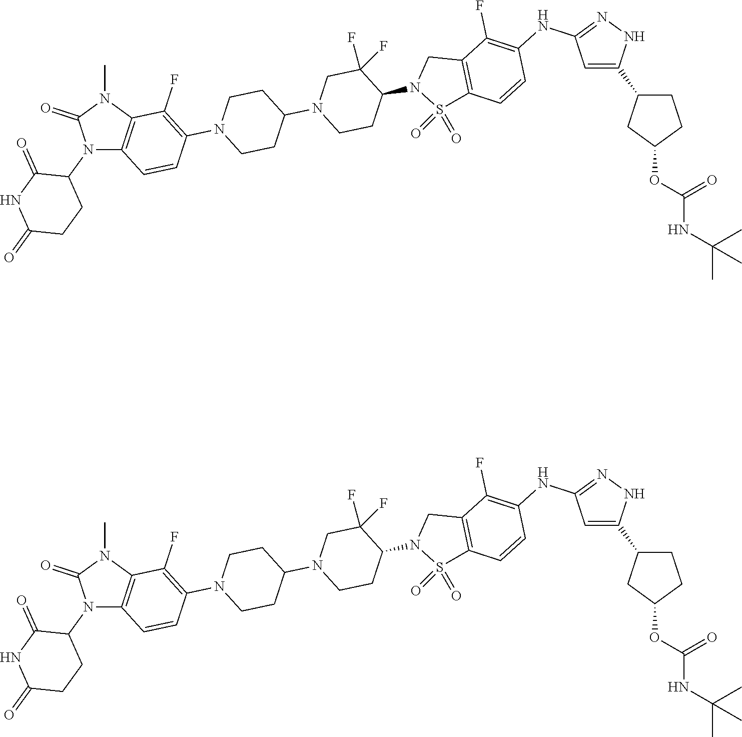 [SMILES: Cn1c(=O)n(C2CCC(=O)NC2=O)c2ccc(N3CCC(N4CC[C@H](N5Cc6c(ccc(Nc7cc([C@@H]8CC[C@H](OC(=O)NC(C)(C)C)C8)[nH]n7)c6F)S5(=O)=O)C(F)(F)C4)CC3)c(F)c21 ; Cn1c(=O)n(C2CCC(=O)NC2=O)c2ccc(N3CCC(N4CC[C@@H](N5Cc6c(ccc(Nc7cc([C@@H]8CC[C@H](OC(=O)NC(C)(C)C)C8)[nH]n7)c6F)S5(=O)=O)C(F)(F)C4)CC3)c(F)c21]

The titled compound was synthesized in the procedures similar to Example 39, which was further separated by Chiral Prep-HPLC to afford:

Enantiomer 1 (Example 58a, 100% ee); Retention time: 5.314 min. ¹H NMR (500 MHz, DMSO) δ 12.00 (s, 1H), 11.10 (s, 1H), 8.82 (s, 1H), 8.33 (t, J=8.0 Hz, 1H), 7.58 (d, J=8.6 Hz, 1H), 6.85 (d, J=8.5 Hz, 1H), 6.81-6.68 (m, 2H), 5.84 (s, 1H), 5.33 (dd, J=12.6, 5.1 Hz, 1H), 5.06-4.96 (m, 1H), 4.75 (d, J=14.2 Hz, 1H), 4.54 (d, J=13.1 Hz, 1H), 4.05-3.90 (m, 1H), 3.47 (s, 3H), 3.25-3.16 (m, 1H), 3.13-2.99 (m, 2H), 2.97-2.61 (m, 10H), 2.45-2.42 (m, 1H), 2.20-2.19 (m, 1H), 2.04-1.96 (m, 2H), 1.94-1.78 (m, 4H), 1.75-1.55 (m, 5H), 1.21 (s, 9H). LC-MS (ESI): m/z [M+H]⁺=929.4;

Enantiomer 1 (Example 58b, 100% ee); Retention time: 11.267, 13.571 min. ¹H NMR (500 MHz, DMSO) δ 12.00 (s, 1H), 11.10 (s, 1H), 8.82 (s, 1H), 8.33 (t, J=8.0 Hz, 1H), 7.58 (d, J=8.6 Hz, 1H), 6.85 (d, J=8.5 Hz, 1H), 6.81-6.68 (m, 2H), 5.84 (s, 1H), 5.33 (dd, J=12.6, 5.1 Hz, 1H), 5.06-4.96 (m, 1H), 4.75 (d, J=14.2 Hz, 1H), 4.54 (d, J=13.1 Hz, 1H), 4.05-3.90 (m, 1H), 3.47 (s, 3H), 3.25-3.16 (m, 1H), 3.13-2.99 (m, 2H), 2.97-2.61 (m, 10H), 2.45-2.42 (m, 1H), 2.20-2.19 (m, 1H), 2.04-1.96 (m, 2H), 1.94-1.78 (m, 4H), 1.75-1.55 (m, 5H), 1.21 (s, 9H). LC-MS (ESI): m/z [M+H]⁺=929.4;

Chiral analytical method: Column: ICHIRALPAK SA 4.6*150 mm 5 μm; Mobile phase: A for MtBE (0.1%2M NH3 MeOH) (%) and B for MeOH:DCM=50:50(%); Gradient: Mobile Phase A: Mobile Phase B=30:70 (v/v); HPLC Equipment: HPLC-Agilent, Back pressure: 100 bar; Column temperature: 35° C.

Chiral Prep-HPLC Condition: CHIRALPAK SA 250*20.0 mml, 5 μm; Mobile phase: A for MtBE (0.1%2M NH3 MeOH) (%) and B for MeOH:DCM=50:50; Gradient: Mobile Phase A: Mobile Phase B=30:70 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 59a and 59b: (1S,3R)-3-(3-((2-((4S)-1′-(1-(2,6-dioxopiperidin-3-yl)-4-fluoro-3-isopropyl-2-oxo-2,3-dihydro-1H-benzo[d]imidazol-5-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl tert-butylcarbamate and (1S,3R)-3-(3-((2-((4R)-1′-(1-(2,6-dioxopiperidin-3-yl)-4-fluoro-3-isopropyl-2-oxo-2,3-dihydro-1H-benzo[d]imidazol-5-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl tert-butylcarbamate

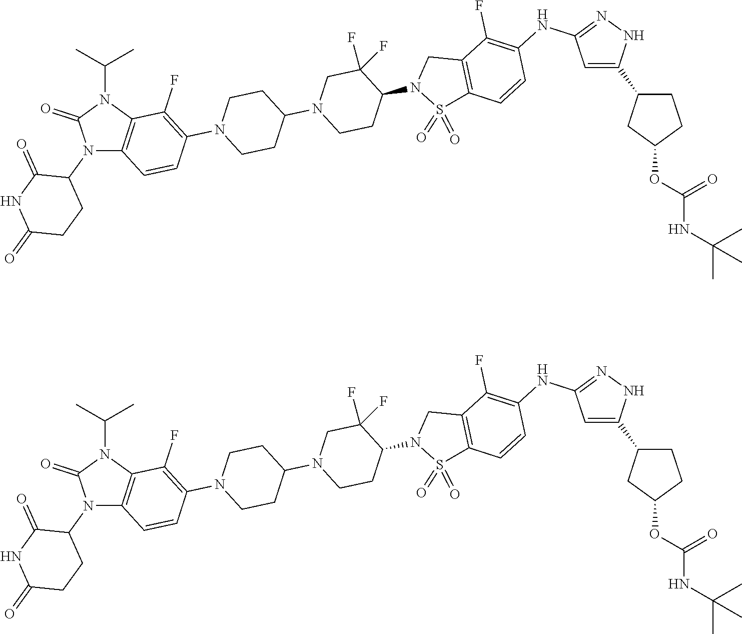 [SMILES: CC(C)n1c(=O)n(C2CCC(=O)NC2=O)c2ccc(N3CCC(N4CC[C@H](N5Cc6c(ccc(Nc7cc([C@@H]8CC[C@H](OC(=O)NC(C)(C)C)C8)[nH]n7)c6F)S5(=O)=O)C(F)(F)C4)CC3)c(F)c21 ; CC(C)n1c(=O)n(C2CCC(=O)NC2=O)c2ccc(N3CCC(N4CC[C@@H](N5Cc6c(ccc(Nc7cc([C@@H]8CC[C@H](OC(=O)NC(C)(C)C)C8)[nH]n7)c6F)S5(=O)=O)C(F)(F)C4)CC3)c(F)c21]

### Step 1: 3-(4-fluoro-3-isopropyl-2-oxo-5-(4-oxopiperidin-1-yl)-2,3-dihydro-1H-benzo[d]imidazol-1-yl)piperidine-2,6-dione

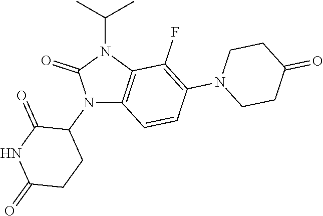 [SMILES: CC(C)n1c(=O)n(C2CCC(=O)NC2=O)c2ccc(N3CCC(=O)CC3)c(F)c21]

The titled compound was synthesized in the procedures similar to Example 37. LC-MS (ESI): m/z [M+H]⁺=403.5;

### Step 2: (1S,3R)-3-(3-((2-((4S)-1′-(1-(2,6-dioxopiperidin-3-yl)-4-fluoro-3-isopropyl-2-oxo-2,3-dihydro-1H-benzo[d]imidazol-5-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl tert-butylcarbamate and (1S,3R)-3-(3-((2-((4R)-1′-(1-(2,6-dioxopiperidin-3-yl)-4-fluoro-3-isopropyl-2-oxo-2,3-dihydro-1H-benzo[d]imidazol-5-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl tert-butylcarbamate

The titled compound was synthesized in the procedures similar to Example 39, which was further separated by Chiral Prep-HPLC to afford:

Enantiomer 1 (Example 59a, 100% ee); Retention time: 6.091 min. ¹H NMR (500 MHz, DMSO) δ 12.01 (s, 1H), 11.09 (s, 1H), 8.82 (s, 1H), 8.33 (t, J=7.7 Hz, 1H), 7.58 (d, J=8.6 Hz, 1H), 6.94 (s, 1H), 6.86 (d, J=8.4 Hz, 1H), 6.79 (t, J=8.0 Hz, 1H), 5.84 (s, 1H), 5.32 (d, J=13.2 Hz, 1H), 5.06-4.96 (m, 1H), 4.76 (dd, J=16.2, 10.5 Hz, 2H), 4.54 (d, J=12.9 Hz, 1H), 4.04-3.92 (m, 1H), 3.65-3.53-3.52 (m, 1H), 3.10-2.97 (m, 2H), 2.95-2.65 (m, 10H), 2.43-2.40 (m, 1H), 2.20-2.10 (m, 1H), 2.07-1.98 (m, 2H), 1.96-1.79 (m, 5H), 1.76-1.56 (m, 6H), 1.42 (d, J=6.8 Hz, 6H), 1.21 (s, 9H). LC-MS (ESI): m/z [M+H]⁺=957.4;

Enantiomer 1 (Example 59a, 100% ee); Retention time: 10.363 min. ¹H NMR (500 MHz, DMSO) δ 12.01 (s, 1H), 11.09 (s, 1H), 8.82 (s, 1H), 8.33 (t, J=7.7 Hz, 1H), 7.58 (d, J=8.6 Hz, 1H), 6.94 (s, 1H), 6.86 (d, J=8.4 Hz, 1H), 6.79 (t, J=8.0 Hz, 1H), 5.84 (s, 1H), 5.32 (d, J=13.2 Hz, 1H), 5.06-4.96 (m, 1H), 4.76 (dd, J=16.2, 10.5 Hz, 2H), 4.54 (d, J=12.9 Hz, 1H), 4.04-3.92 (m, 1H), 3.65-3.53-3.52 (m, 1H), 3.10-2.97 (m, 2H), 2.95-2.65 (m, 10H), 2.43-2.40 (m, 1H), 2.20-2.10 (m, 1H), 2.07-1.98 (m, 2H), 1.96-1.79 (m, 5H), 1.76-1.56 (m, 6H), 1.42 (d, J=6.8 Hz, 6H), 1.21 (s, 9H). LC-MS (ESI): m/z [M+H]⁺=957.4;

Chiral analytical method: Column: ICHIRALPAK IE 4.6*150 mm 5 μm; Mobile phase: A for MtBE (0.1%2M NH3 MeOH) (%) and B for MeOH:DCM=50:50(%); Gradient: Mobile Phase A: Mobile Phase B=20:80 (v/v); HPLC Equipment: HPLC-Agilent, Back pressure: 100 bar; Column temperature: 35° C.

Chiral Prep-HPLC Condition: CHIRALPAK IE 250*20.0 mml, 5 μm; Mobile phase: A for MtBE (0.1%2M NH3 MeOH) (%) and B for MeOH:DCM=50:50; Gradient: Mobile Phase A: Mobile Phase B=20:80 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 60: (1S,3R)-3-(3-((2-((S)-1′-(1-((S)-2,6-dioxopiperidin-3-yl)-7-fluoro-3-methyl-2-oxo-2,3-dihydro-1H-benzo[d]imidazol-5-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl tert-butylcarbamate

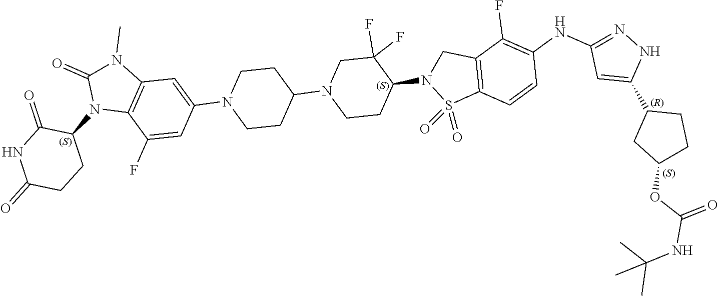

### Step 1: 3-(2,6-bis(benzyloxy)pyridin-3-yl)-4-fluoro-1-methyl-6-(1,4-dioxa-8-azaspiro[4.5]decan-8-yl)-1,3-dihydro-2H-benzo[d]imidazol-2-one

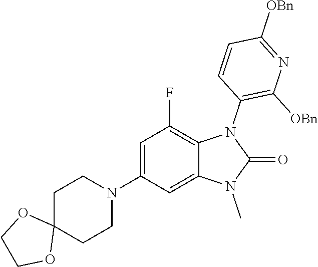 [SMILES: Cn1c(=O)n(-c2ccc(OCc3ccccc3)nc2OCc2ccccc2)c2c(F)cc(N3CCC4(CC3)OCCO4)cc21]

To a solution of 3-(2,6-bis(benzyloxy)pyridin-3-yl)-6-bromo-4-fluoro-1-methyl-1,3-dihydro-2H-benzo[d]imidazol-2-one (1.8 g, 3.38 mmol) in dry-dioxane (20 mL) were added 1,4-dioxa-8-azaspiro[4.5]decane (725 mg, 5.07 mmol), Pd-PEPPSI-IPentCl (142 mg, 0.17 mmol) and Cs₂CO₃ (2.2 g, 6.76 mmol) at 100° C. for 2 h under N₂ atmosphere. The resulting mixture was concentrated under vacuum. The residue was purified by combi-flash (PE/EA=100:0 to 49:51) to give the product (1.8 g, 89.5%) as yellow oil.

### Step 2: 3-(7-fluoro-3-methyl-2-oxo-5-(1,4-dioxa-8-azaspiro[4.5]decan-8-yl)-2,3-dihydro-1H-benzo[d]imidazol-1-yl)piperidine-2,6-dione

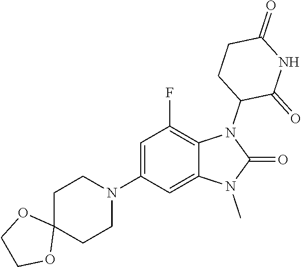 [SMILES: Cn1c(=O)n(C2CCC(=O)NC2=O)c2c(F)cc(N3CCC4(CC3)OCCO4)cc21]

To a solution of 3-(2,6-bis(benzyloxy)pyridin-3-yl)-4-fluoro-1-methyl-6-(1,4-dioxa-8-azaspiro[4.5]decan-8-yl)-1,3-dihydro-2H-benzo[d]imidazol-2-one (1.8 g, 3.02 mmol) in dry-THF (100 mL) was added Pd/C (1.8 g). The mixture was hydrogenated at room temperature for overnight under hydrogen atmosphere using a hydrogen balloon. The resulting mixture was filtered. The filter cake was washed with THF (100 mL). The filtrate was concentrated under vacuum. The residue was without purified to give the product (1.1 g, 87.3%) as light brown solid.

### Step 3: 3-(7-fluoro-3-methyl-2-oxo-5-(4-oxopiperidin-1-yl)-2,3-dihydro-1H-benzo[d]imidazol-1-yl)piperidine-2,6-dione

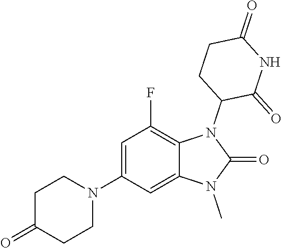 [SMILES: Cn1c(=O)n(C2CCC(=O)NC2=O)c2c(F)cc(N3CCC(=O)CC3)cc21]

To a solution of 3-(7-fluoro-3-methyl-2-oxo-5-(1,4-dioxa-8-azaspiro[4.5]decan-8-yl)-2,3-dihydro-1H-benzo[d]imidazol-1-yl)piperidine-2,6-dione (1 g, 2.39 mmol) in H₂SO₄ (10 eq, 23.9 mmol) at 0° C. The reaction was stirred at rt for 2 h. The resulting mixture was quenched with ice-water (20 mL). The mixture was acidified to PH=7 with NaHCO₃ solution. The resulting mixture was extracted with EA. The organic layers were concentrated under vacuum. The resulting mixture was added ether (15 mL) stirred at rt for 1 h and filtered, the filter cake was washed with ether. The residue was without purified to give the product (788.3 mg, 89.4%) as light brown solid. LCMS (ESI) m/e [M+1]⁺=375.

### Step 4: (1S,3R)-3-(3-((2-((S)-1′-(1-((S)-2,6-dioxopiperidin-3-yl)-7-fluoro-3-methyl-2-oxo-2,3-dihydro-1H-benzo[d]imidazol-5-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl tert-butylcarbamate

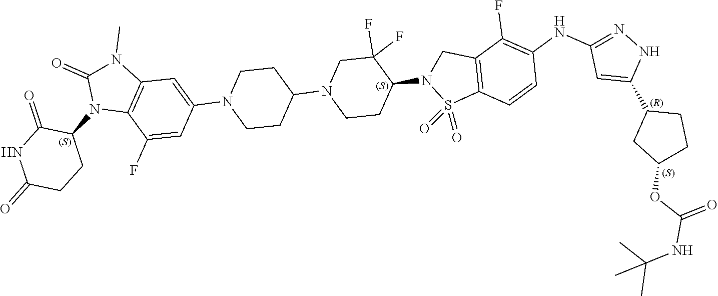

The titled compound was synthesized in the procedures similar to Example 41 in a racemic form, which was further separated by Chiral Prep-HPLC to afford Example 60 (100% ee); Retention time: 6.492 min. ¹H NMR (500 MHz, DMSO) δ 12.00 (s, 1H), 11.07 (s, 1H), 8.82 (s, 1H), 8.33 (t, J=7.9 Hz, 1H), 7.58 (d, J=8.7 Hz, 1H), 6.76 (s, 1H), 6.70 (s, 1H), 6.54 (d, J=15.1 Hz, 1H), 5.84 (s, 1H), 5.41 (s, 1H), 4.98 (s, 1H), 4.74 (d, J=14.4 Hz, 1H), 4.53 (d, J=13.0 Hz, 1H), 4.03-3.92 (m, 1H), 3.73 (d, J=11.2 Hz, 2H), 3.34 (s, 3H), 3.24-3.17 (m, 1H), 3.11-2.91 (m, 3H), 2.82-2.57 (m, 7H), 2.28-1.99 (m, 4H), 1.93-1.78 (m, 4H), 1.75-1.67 (m, 2H), 1.64-1.51 (m, 3H), 1.21 (s, 9H). LCMS (ESI) m/e [M+1]⁺=929.47

Chiral analytical method: Column: Amylose-SA 4.6*250 mm 5 μm; Mobile phase: A for MtBE (0.1%2M NH3 MeOH)(%) and B for MeOH:DCM=50:50 (v/v); Gradient: Mobile Phase A: Mobile Phase B=30:70 (v/v); HPLC Equipment: Agilent 1260 Infinity II (ARD_LC-808); Column temperature: 25° C.

Chiral Prep-HPLC Condition: CHIRALPAK IE 250*20.0 mml, 5 μm; Mobile phase: A for MtBE (0.1%2M NH3 MeOH)(%) and B for MeOH:DCM=50:50; Gradient: Mobile Phase A: Mobile Phase B=30:70 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 61: (1S,3R)-3-(3-((2-((S)-1-((1-(1-((S)-2,6-dioxopiperidin-3-yl)-4,7-difluoro-3-methyl-2-oxo-2,3-dihydro-1H-benzo[d]imidazol-5-yl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl tert-butylcarbamate

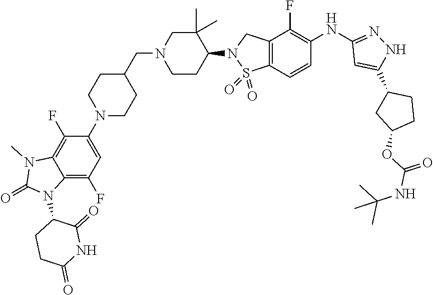 [SMILES: Cn1c(=O)n([C@H]2CCC(=O)NC2=O)c2c(F)cc(N3CCC(CN4CC[C@H](N5Cc6c(ccc(Nc7cc([C@@H]8CC[C@H](OC(=O)NC(C)(C)C)C8)[nH]n7)c6F)S5(=O)=O)C(C)(C)C4)CC3)c(F)c21]

The titled compound was synthesized in the procedures similar to Example 55 in a racemic form, which was further separated by Chiral Prep-HPLC to afford Example 61 (100% ee); Retention time: 5.97 min. ¹H NMR (500 MHz, DMSO) δ 11.98 (s, 1H), 11.11 (s, 1H), 8.77 (s, 1H), 8.31 (t, J=8.1 Hz, 1H), 7.54 (d, J=8.6 Hz, 1H), 6.71 (d, J=48.5 Hz, 2H), 5.83 (s, 1H), 5.47 (s, 1H), 4.97 (s, 1H), 4.54 (s, 2H), 3.50 (s, 3H), 3.28-3.20 (m, 3H), 3.10-3.02 (m, 1H), 2.96 (s, 2H), 2.67-2.60 (m, 8H), 2.23-2.08 (m, 3H), 2.01-1.96 (m, 3H), 1.90 (s, 1H), 1.82-1.80 (m, 2H), 1.73-1.71 (m, 1H), 1.61 (s, 2H), 1.52 (s, 1H), 1.32-1.23 (m, 2H), 1.21 (s, 9H), 1.12 (s, 3H), 0.94 (s, 3H). LC-MS (ESI): m/z [M+H]⁺=953.27.

Chiral analytical method: Column: I-Cellulose-5, 4.6 mm×250 mm, 5 um; Mobile phase: A for Hexane and B for EtOH (0.1% 2M NH₃ MeOH); Gradient: Mobile Phase A: Mobile Phase B=30:70 (v/v); HPLC Equipment: HPLC-Agilent, Back pressure: 100 bar; Column temperature: 35° C.

Chiral Prep-HPLC Condition: Column: I-Cellulose-5, 21.2 mm×250 mm, 5 um; Mobile phase: A for Hexane and B for EtOH (0.2% 2M NH₃ MeOH); Gradient: Mobile Phase A: Mobile Phase B=30:70 (v/v); Flow Rate: 20 mL/min, Wave Length: UV 200 nm and 270 nm, Prep-HPLC Equipment: Prep-HPLC-Gilson, Back pressure: 100 bar; Column temperature: 25° C.

### Example 62: (1S,3R)-3-(3-((2-(1-((1-(3-(2,6-dioxopiperidin-3-yl)-2-oxo-2,3-dihydrobenzo[d]oxazol-6-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

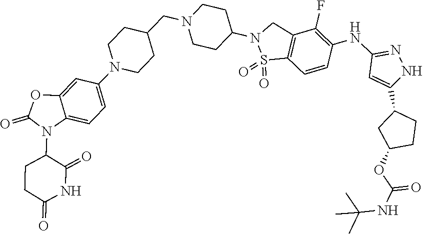 [SMILES: CC(C)(C)NC(=O)O[C@H]1CC[C@@H](c2cc(Nc3ccc4c(c3F)CN(C3CCN(CC5CCN(c6ccc7c(c6)oc(=O)n7C6CCC(=O)NC6=O)CC5)CC3)S4(=O)=O)n[nH]2)C1]

### Step 1: 3-(2,6-bis(benzyloxy)pyridin-3-yl)-6-bromobenzo[d]oxazol-2(3H)-one

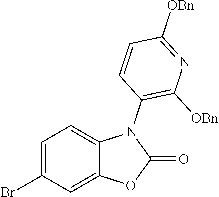 [SMILES: O=c1oc2cc(Br)ccc2n1-c1ccc(OCc2ccccc2)nc1OCc1ccccc1]

To a solution of 6-bromo-3H-1,3-benzoxazol-2-one (42 g, 196 mmol) in dioxane (1000 mL) was added 2,6-bis(benzyloxy)-3-(4,4,5,5-tetramethyl-1,3,2-dioxaborolan-2-yl)pyridine (122.7 g, 294 mmol), Cu(OAc)₂ (35.5 g, 196 mmol) and pyridine (46 g, 588 mmol). The mixture was stirred under 02 atmosphere at 80° C. for overnight. The mixture was filtered and concentrated under reduced pressure. The residue was purified by silica gel chromatography, eluting with DCM/PE (1:10; Rf=0.3) to afford the product (55 g, 56% yield). LC-MS (ESI): m/z [M+H^{]+}=503.2

### Step 2: 6-(4-(1,3-dioxolan-2-yl)piperidin-1-yl)-3-(2,6-bis(benzyloxy)pyridin-3-yl)benzo[d]oxazol-2(3H)-one

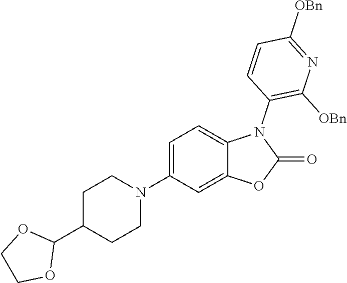 [SMILES: O=c1oc2cc(N3CCC(C4OCCO4)CC3)ccc2n1-c1ccc(OCc2ccccc2)nc1OCc1ccccc1]

To a solution of 3-(2,6-bis(benzyloxy)pyridin-3-yl)-6-bromobenzo[d]oxazol-2(3H)-one (3.3 g, 6.57 mmol) and 4-(1,3-dioxolan-2-yl)piperidine (1.03 mmol) in dioxane (50 mL) was added Pd₂dba₃·DCM (600 mg, 0.66 mmol) RuPhos (613 mg, 1.31 mmol) and Cs₂CO₃ (6.4 g, 19.71 mmol), the reaction mixture was stirred for 3 hours at 110° C. under nitrogen. The resulting mixture was cooled to room temperature, filtered, the filtrated was concentrated under reduced pressure. The residue was purified by silica gel column chromatography, eluting with PE/EA (1:1-EA) to afford the product (2.18 mg, 57% yield). LC-MS (ESI): m/z [M+H]⁺=580.3.

### Step 3: 3-(6-(4-(1,3-dioxolan-2-yl)piperidin-1-yl)-2-oxobenzo[d]oxazol-3(2H)-yl)piperidine-2,6-dione

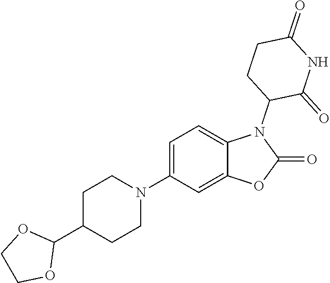 [SMILES: O=C1CCC(n2c(=O)oc3cc(N4CCC(C5OCCO5)CC4)ccc32)C(=O)N1]

To a solution of 6-(4-(1,3-dioxolan-2-yl)piperidin-1-yl)-3-(2,6-bis(benzyloxy)pyridin-3-yl)benzo[d]oxazol-2(3H)-one (2.18 g, 3.77 mmol) in dioxane (50 mL) was added Pd/C (10% on carbon, wet, 500 mg) and Pd(OH)2/C (10% on carbon, wet, 500 mg). The suspension was degassed and purged with hydrogen 3 times. The resulting mixture was stirred at 50° C. under a hydrogen balloon for 16 h. The mixture was cooled to room temperature, filtered, and the filter cake was washed with EA (20 mL×3). The filtrate was concentrated under reduced pressure to afford the product (1.5 g, 99% yield). LC-MS (ESI): m/z [M+H]⁺=402.4.

### Step 4: 1-(3-(2,6-dioxopiperidin-3-yl)-2-oxo-2,3-dihydrobenzo[d]oxazol-6-yl)piperidine-4-carbaldehyde

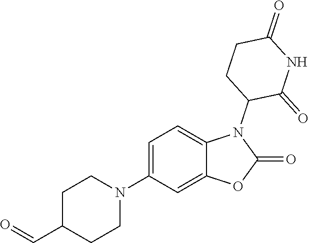 [SMILES: O=CC1CCN(c2ccc3c(c2)oc(=O)n3C2CCC(=O)NC2=O)CC1]

3-(6-(4-(1,3-dioxolan-2-yl)piperidin-1-yl)-2-oxobenzo[d]oxazol-3(2H)-yl)piperidine-2,6-dione (157 mg, 0.39 mmol) was dissolved in formic acid (3 mL), the reaction mixture was stirred for 16 hours at room temperature. The resulting mixture was concentrated under reduce pressure, dried over in vacuum, to afford the crude product without further purification (143 mg, >100% yield). LC-MS (ESI): m/z [M+H]⁺=358.2.

### Step 5: 1-(3-(2,6-dioxopiperidin-3-yl)-2-oxo-2,3-dihydrobenzo[d]oxazol-6-yl)piperidine-4-carbaldehyde

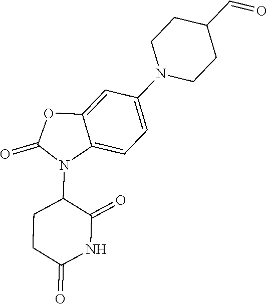 [SMILES: O=CC1CCN(c2ccc3c(c2)oc(=O)n3C2CCC(=O)NC2=O)CC1]

### Step 6: (1S,3R)-3-(3-((2-(1-((1-(3-(2,6-dioxopiperidin-3-yl)-2-oxo-2,3-dihydrobenzo[d]oxazol-6-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

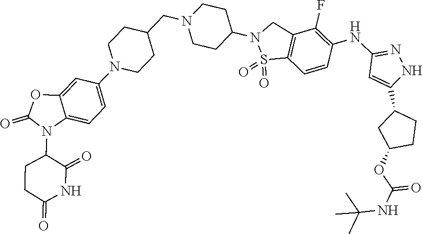 [SMILES: CC(C)(C)NC(=O)O[C@H]1CC[C@@H](c2cc(Nc3ccc4c(c3F)CN(C3CCN(CC5CCN(c6ccc7c(c6)oc(=O)n7C6CCC(=O)NC6=O)CC5)CC3)S4(=O)=O)n[nH]2)C1]

The titled compound was synthesized in the procedures similar to Example 36. LC-MS (ESI): m/z [M+H]⁺=876.57; ¹H NMR (500 MHz, DMSO) δ=11.97 (s, 1H), 11.16 (s, 1H), 8.75 (s, 1H), 8.28 (s, 1H), 7.50 (d, J=8.0, 1H), 7.06 (d, J=13.4, 2H), 6.79-6.70 (m, 2H), 5.83 (s, 1H), 5.29 (d, J=12.3, 1H), 4.98 (s, 1H), 4.51 (s, 2H), 3.59 (d, J=11.7, 2H), 3.42 (m, 1H), 3.06 (m, 1H), 2.88 (m, 3H), 2.64 (m, 4H), 2.36 (m, 2H), 2.13 (m, 1H), 2.02 (m, 3H), 1.89 (m, 5H), 1.78 (m, 2H), 1.72 (m, 2H), 1.62 (m, 2H), 1.21 (m, 12H).

### Example 63: (1S,3R)-3-(3-((2-(1′-(3-(2,6-dioxopiperidin-3-yl)-2-oxo-2,3-dihydrobenzo[d]oxazol-6-yl)-[1,4′-bipiperidin]-4-yl)-4-fluoro-1,1-dioxido-2,3-dihydrobenzo[d]isothiazol-5-yl)amino)-1H-pyrazol-5-yl)cyclopentyl tert-butylcarbamate

### Step 1:3-(2-oxo-6-(1,4-dioxa-8-azaspiro[4.5]decan-8-yl)benzo[d]oxazol-3(2H)-yl)piperidine-2,6-dione

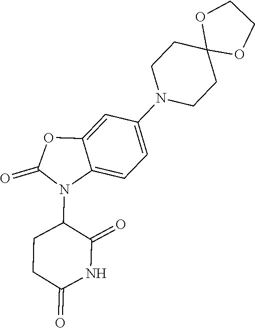 [SMILES: O=C1CCC(n2c(=O)oc3cc(N4CCC5(CC4)OCCO5)ccc32)C(=O)N1]

### Step 2:3-(2-oxo-6-(4-oxopiperidin-1-yl)benzo[d]oxazol-3(2H)-yl)piperidine-2,6-dione

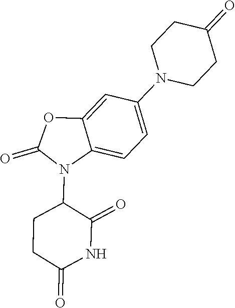 [SMILES: O=C1CCN(c2ccc3c(c2)oc(=O)n3C2CCC(=O)NC2=O)CC1]

### Step 3: (1S,3R)-3-(3-((2-(1′-(3-(2,6-dioxopiperidin-3-yl)-2-oxo-2,3-dihydrobenzo[d]oxazol-6-yl)-[1,4′-bipiperidin]-4-yl)-4-fluoro-1,1-dioxido-2,3-dihydrobenzo[d]isothiazol-5-yl)amino)-1H-pyrazol-5-yl)cyclopentyl tert-butylcarbamate

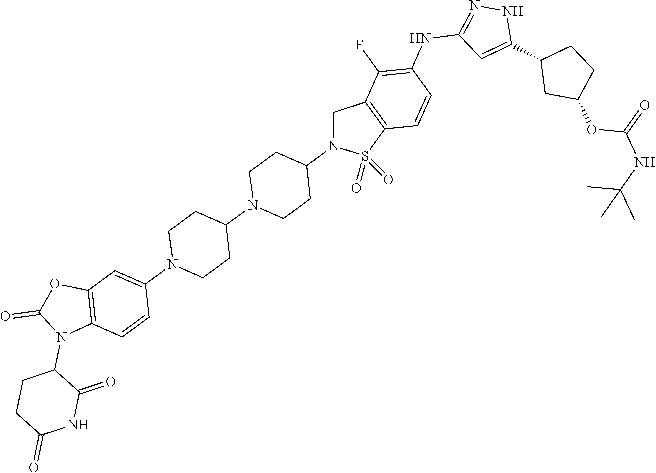 [SMILES: CC(C)(C)NC(=O)O[C@H]1CC[C@@H](c2cc(Nc3ccc4c(c3F)CN(C3CCN(C5CCN(c6ccc7c(c6)oc(=O)n7C6CCC(=O)NC6=O)CC5)CC3)S4(=O)=O)n[nH]2)C1]

The titled compound was synthesized in the procedures similar to Example 39. ¹H NMR (500 MHz, DMSO-d₆) δ 11.98 (s, 1H), 11.17 (s, 1H), 8.76 (s, 1H), 8.28 (s, 1H), 7.51 (d, J=5 Hz, 1H), 7.15-7.06 (s, 2H), 6.85-6.76 (m, 2H), 5.83 (s, 1H), 5.46-5.28 (m, 1H), 4.97 (s, 1H), 4.51 (s, 2H), 3.68-2.60 (m, 2H), 3.55-3.52 (m, 1H), 3.09-3.01 (m, 1H), 3.01-2.94 (m, 2H), 2.98-2.82 (m, 1H), 2.72-2.58 (m, 4H), 2.47-2.36 (m, 2H), 2.33-2.23 (m, 2H), 2.17-2.08 (m, 1H), 2.06-1.98 (m, 1H), 1.97-1.87 (m, 3H), 1.87-1.77 (m, 4H), 1.76-1.65 (m, 2H), 1.63-1.50 (m, 3H), 1.21 (s, 9H). LC-MS (ESI): m/z [M+H]⁺=862.

### Example 64: (1S,3R)-3-(3-((2-(1-(1-(3-(2,6-dioxopiperidin-3-yl)-2-oxo-2,3-dihydrobenzo[d]oxazol-7-yl)azetidin-3-yl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

### Step 1: 3-(7-bromo-2-oxobenzo[d]oxazol-3(2H)-yl)piperidine-2,6-dione

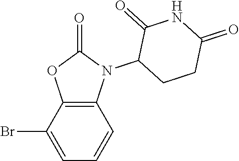 [SMILES: O=C1CCC(n2c(=O)oc3c(Br)cccc32)C(=O)N1]

To a solution of 7-bromobenzo[d]oxazol-2(3H)-one (1 g, 4.7 mmo) in DMF (15 mL) were added Cs₂CO₃ (3.1 g, 9.39 mmol) and 3-bromopiperidine-2,6-dione (1.8 g, 9.39 mmol). The mixture was stirred at 50° C. for 16 h. The mixture was poured into ice water (100 mL), and filtered. The solids were collected and purified by silica gel chromatography, eluting with PE/EA (1:7; Rf=0.3) to afford the product (400 mg, 26% yield). LC-MS (ESI): m/z [M+H]⁺=325.

### Step 2: 3-(2-oxo-7-(5,8-dioxa-2-azaspiro[3.4]octan-2-yl)benzo[d]oxazol-3(2H)-yl)piperidine-2,6-dione

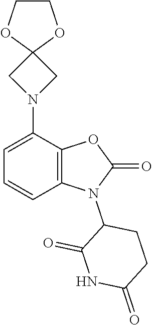 [SMILES: O=C1CCC(n2c(=O)oc3c(N4CC5(C4)OCCO5)cccc32)C(=O)N1]

A mixture of 3-(7-bromo-2-oxobenzo[d]oxazol-3(2H)-yl)piperidine-2,6-dione (400 mg, 1.23 mmol), 5,8-dioxa-2-azaspiro[3.4]octane hydrochloride (224 mg, 1.5 mmol), Pd-PEPPSI-Iheptcl (52 mg, 0.06 mmol) and Cs₂CO₃ (1.2 g, 3.69 mmol) in dioxane was stirred at 100° C. for 16 h under a nitrogen atmosphere. The mixture was concentrated under vacuum and the residue was purified by silica gel chromatography, eluting with EA/PE (100%; Rf=0.4) to afford the product (50 mg, 9% yield). LC-MS (ESI): m/z [M+H]⁺=360.

### Step 3: 3-(2-oxo-7-(3-oxoazetidin-1-yl)benzo[d]oxazol-3(2H)-yl)piperidine-2,6-dione

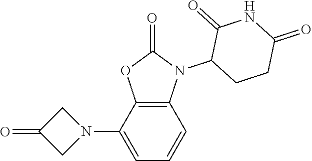 [SMILES: O=C1CN(c2cccc3c2oc(=O)n3C2CCC(=O)NC2=O)C1]

A mixture of 3-(2-oxo-7-(5,8-dioxa-2-azaspiro[3.4]octan-2-yl)benzo[d]oxazol-3(2H)-yl)piperidine-2,6-dione (50 mg, 0.14 mmol) in con·H2SO4 (1 mL) was stirred at rt for 4 h. The mixture was quenched by sat·NaHCO₃ (20 mL), extracted with EA (3×30 mL). The combined organic layers were dried over anhydrous Na2SO4, then filtered and concentrated under vacuum to afford the product (50 mg, 9% yield). LC-MS (ESI): m/z [M+H]⁺=316.

### Step 4: (1S,3R)-3-(3-((2-(1-(1-(3-(2,6-dioxopiperidin-3-yl)-2-oxo-2,3-dihydrobenzo[d]oxazol-7-yl)azetidin-3-yl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

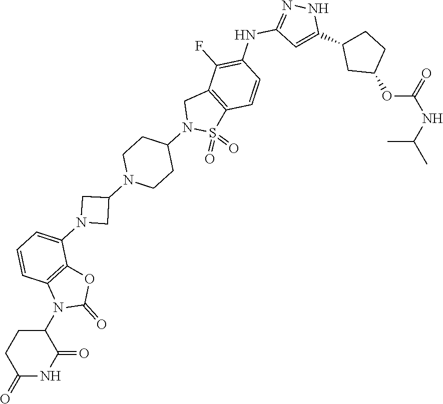 [SMILES: CC(C)NC(=O)O[C@H]1CC[C@@H](c2cc(Nc3ccc4c(c3F)CN(C3CCN(C5CN(c6cccc7c6oc(=O)n7C6CCC(=O)NC6=O)C5)CC3)S4(=O)=O)n[nH]2)C1]

The titled compound was synthesized in the procedures similar to Example 63. ¹H NMR (500 MHz, DMSO-d₆) δ 11.98 (s, 1H), 11.19 (s, 1H), 8.76 (s, 1H), 8.29 (s, 1H), 7.51 (d, J=10 Hz, 1H), 7.02 (t, J=10 Hz, 1H), 6.79-6.72 (m, 1H), 6.59 (d, J=10 Hz, 1H), 6.27 (d, J=10 Hz, 1H), 5.83 (s, 1H), 5.33-5.26 (m, 1H), 5.01-4.94 (m, 1H), 4.51 (s, 2H), 4.11 (t, J=5 Hz, 2H), 3.78 (t, J=5 Hz, 2H), 3.52-3.43 (m, 1H), 3.11-3.02 (m, 1H), 2.94-2.82 (m, 3H), 2.71-2.59 (m, 3H), 2.47-2.36 (m, 1H), 2.17-2.08 (m, 1H), 2.08-1.99 (m, 3H), 1.97-1.80 (m, 5H), 1.77-1.68 (m, 2H), 1.62-1.53 (m, 1H), 1.21 (s, 9H). LC-MS (ESI): m/z [M+H]⁺=834.

### Example 65a and 65b: (1S,3R)-3-(3-((2-(1-((1-(3-((S)-2,6-dioxopiperidin-3-yl)-2-oxo-2,3-dihydrobenzo[d]oxazol-6-yl)piperidin-4-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl tert-butylcarbamate and (1S,3R)-3-(3-((2-(1-((1-(3-((R)-2,6-dioxopiperidin-3-yl)-2-oxo-2,3-dihydrobenzo[d]oxazol-6-yl)piperidin-4-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl tert-butylcarbamate

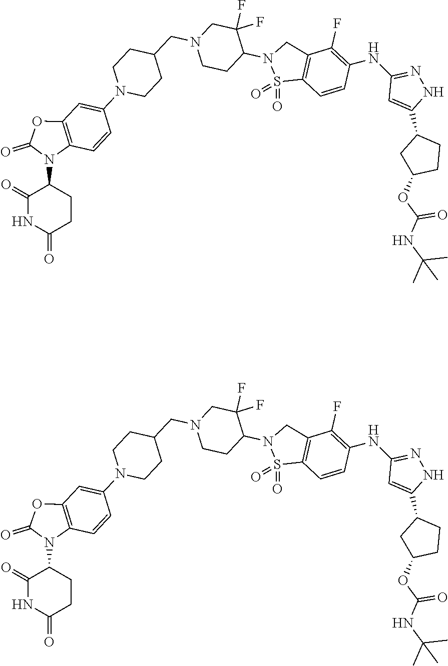 [SMILES: CC(C)(C)NC(=O)O[C@H]1CC[C@@H](c2cc(Nc3ccc4c(c3F)CN(C3CCN(CC5CCN(c6ccc7c(c6)oc(=O)n7[C@H]6CCC(=O)NC6=O)CC5)CC3(F)F)S4(=O)=O)n[nH]2)C1 ; CC(C)(C)NC(=O)O[C@H]1CC[C@@H](c2cc(Nc3ccc4c(c3F)CN(C3CCN(CC5CCN(c6ccc7c(c6)oc(=O)n7[C@@H]6CCC(=O)NC6=O)CC5)CC3(F)F)S4(=O)=O)n[nH]2)C1]

The titled compound was synthesized in the procedures similar to Example 62 in a racemic form, which was further separated by Chiral Prep-HPLC to afford:

Enantiomer 1 (Example 65a, 100% ee); Retention time: 6.964 min. ¹H NMR (500 MHz, DMSO) δ 12.00 (s, 1H), 11.17 (s, 1H), 8.83 (s, 1H), 8.33 (d, J=8.7 Hz, 1H), 7.58 (d, J=8.6 Hz, 1H), 7.07 (d, J=9.0 Hz, 2H), 6.78 (d, J=9.5 Hz, 2H), 5.84 (s, 1H), 5.29 (dd, J=12.7, 5.3 Hz, 1H), 5.06-4.96 (m, 1H), 5.06-4.96 (m, 1H) 4.75 (d, J=14.5 Hz, 1H), 4.54 (d, J=13.3 Hz, 1H), 4.06-3.92 (m, 1H), 3.61 (d, J=11.4 Hz, 2H), 3.22-3.12 (m, 1H), 3.10-3.02 (m, 1H), 2.98-2.84 (m, 3H), 2.66-2.63 (m, 3H), 2.44-2.41 (m, 3H), 2.34-2.27 (m, 3H), 2.24-2.10 (m, 2H), 2.08-1.98 (m, 1H), 1.95-1.86 (m, 1H), 1.85-1.53 (m, 8H), 1.21 (s, 12H). LC-MS (ESI): m/z [M+H]⁺=912.4;

Enantiomer 1 (Example 65b, 100% ee); Retention time: 8.696 min. ¹H NMR (500 MHz, DMSO) δ 12.00 (s, 1H), 11.17 (s, 1H), 8.83 (s, 1H), 8.33 (d, J=8.7 Hz, 1H), 7.58 (d, J=8.6 Hz, 1H), 7.07 (d, J=9.0 Hz, 2H), 6.78 (d, J=9.5 Hz, 2H), 5.84 (s, 1H), 5.29 (dd, J=12.7, 5.3 Hz, 1H), 5.06-4.96 (m, 1H), 5.06-4.96 (m, 1H) 4.75 (d, J=14.5 Hz, 1H), 4.54 (d, J=13.3 Hz, 1H), 4.06-3.92 (m, 1H), 3.61 (d, J=11.4 Hz, 2H), 3.22-3.12 (m, 1H), 3.10-3.02 (m, 1H), 2.98-2.84 (m, 3H), 2.66-2.63 (m, 3H), 2.44-2.41 (m, 3H), 2.34-2.27 (m, 3H), 2.24-2.10 (m, 2H), 2.08-1.98 (m, 1H), 1.95-1.86 (m, 1H), 1.85-1.53 (m, 8H), 1.21 (s, 12H). LC-MS (ESI): m/z [M+H]⁺=912.4;

Chiral analytical method: Column: ICHIRALPAK IC-5 4.6*150 mm 5 μm; Mobile phase: A for MtBE (0.1%2M NH3 MeOH) (%) and B for MeOH:DCM=50:50(%); Gradient: Mobile Phase A: Mobile Phase B=50:50 (v/v); HPLC Equipment: HPLC-Agilent, Back pressure: 100 bar; Column temperature: 35° C.

Chiral Prep-HPLC Condition: CHIRALPAK IC-5 250*20.0 mml, 5 μm; Mobile phase: A for MtBE (0.1%2M NH3 MeOH) (%) and B for MeOH:DCM=50:50; Gradient: Mobile Phase A: Mobile Phase B=50:50 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 66a and 66b: (1S,3R)-3-(3-((2-((4S)-1-((1-(3-(2,6-dioxopiperidin-3-yl)-2-oxo-2,3-dihydrobenzo[d]oxazol-7-yl)piperidin-4-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl tert-butylcarbamate and (1S,3R)-3-(3-((2-((4R)-1-((1-(3-(2,6-dioxopiperidin-3-yl)-2-oxo-2,3-dihydrobenzo[d]oxazol-7-yl)piperidin-4-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl tert-butylcarbamate

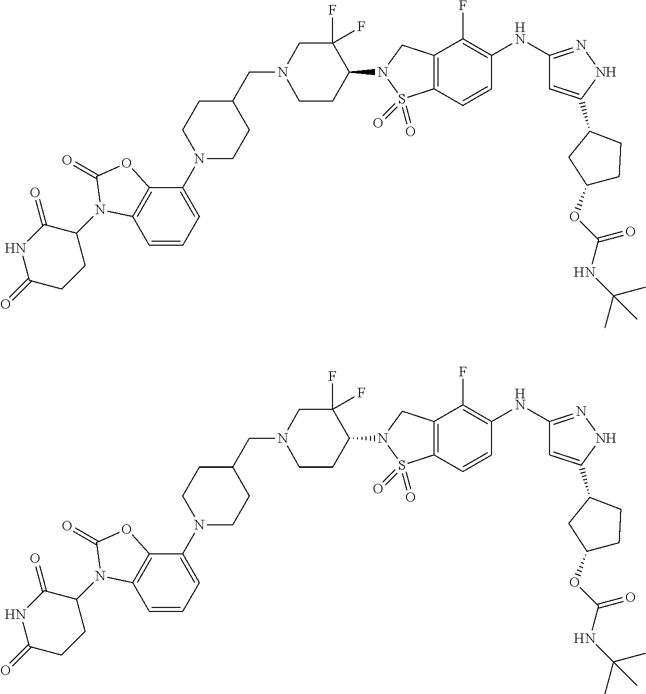 [SMILES: CC(C)(C)NC(=O)O[C@H]1CC[C@@H](c2cc(Nc3ccc4c(c3F)CN([C@H]3CCN(CC5CCN(c6cccc7c6oc(=O)n7C6CCC(=O)NC6=O)CC5)CC3(F)F)S4(=O)=O)n[nH]2)C1 ; CC(C)(C)NC(=O)O[C@H]1CC[C@@H](c2cc(Nc3ccc4c(c3F)CN([C@@H]3CCN(CC5CCN(c6cccc7c6oc(=O)n7C6CCC(=O)NC6=O)CC5)CC3(F)F)S4(=O)=O)n[nH]2)C1]

The titled compound was synthesized in the procedures similar to Example 64 in a racemic form, which was further separated by Chiral Prep-HPLC to afford:

Enantiomer 1 (Example 66a, 100% ee); Retention time: 5.192, 5.937 min. ¹H NMR (500 MHz, DMSO) δ 12.00 (s, 1H), 11.20 (s, 1H), 8.83 (s, 1H), 8.34 (t, J=8.1 Hz, 1H), 7.58 (d, J=8.6 Hz, 1H), 7.08 (t, J=8.1 Hz, 1H), 6.79-6.71 (m, 3H), 5.84 (s, 1H), 5.32 (dd, J=12.7, 5.4 Hz, 1H), 5.06-4.96 (m, 1H), 4.76 (d, J=14.4 Hz, 1H), 4.54 (d, J=13.4 Hz, 1H), 4.06-3.92 (m, 1H), 3.74-3.64 (m, 2H), 3.22-3.14 (m, 1H), 3.10-3.02 (m, 1H), 3.00-2.92 (m, 1H), 2.91-2.85 (m, 1H), 2.77 (s, 3H), 2.67 (d, J=5.5 Hz, 2H), 2.44-2.41 (m, 1H), 2.35-2.27 (m, 3H), 2.25-2.11 (m, 2H), 2.07-1.99 (m, 1H), 1.95-1.87 (m, 1H), 1.86-1.78 (m, 3H), 1.77-1.66 (m, 3H), 1.62-1.54 (m, 1H), 1.31-1.24 (m, 2H), 1.21 (s, 9H). LC-MS (ESI): m/z [M+H]⁺=912.4;

Enantiomer 1 (Example 66b, 100% ee); Retention time: 8.439, 9.076 min. ¹H NMR (500 MHz, DMSO) δ 12.00 (s, 1H), 11.20 (s, 1H), 8.83 (s, 1H), 8.34 (t, J=8.1 Hz, 1H), 7.58 (d, J=8.6 Hz, 1H), 7.08 (t, J=8.1 Hz, 1H), 6.79-6.71 (m, 3H), 5.84 (s, 1H), 5.32 (dd, J=12.7, 5.4 Hz, 1H), 5.06-4.96 (m, 1H), 4.76 (d, J=14.4 Hz, 1H), 4.54 (d, J=13.4 Hz, 1H), 4.06-3.92 (m, 1H), 3.74-3.64 (m, 2H), 3.22-3.14 (m, 1H), 3.10-3.02 (m, 1H), 3.00-2.92 (m, 1H), 2.91-2.85 (m, 1H), 2.77 (s, 3H), 2.67 (d, J=5.5 Hz, 2H), 2.44-2.41 (m, 1H), 2.35-2.27 (m, 3H), 2.25-2.11 (m, 2H), 2.07-1.99 (m, 1H), 1.95-1.87 (m, 1H), 1.86-1.78 (m, 3H), 1.77-1.66 (m, 3H), 1.62-1.54 (m, 1H), 1.31-1.24 (m, 2H), 1.21 (s, 9H). LC-MS (ESI): m/z [M+H]⁺=912.4;

Chiral analytical method: Column: ICHIRALPAK IE 4.6*150 mm 5 μm; Mobile phase: A for MtBE (0.1%2M NH3 MeOH) (%) and B for MeOH:DCM=50:50(%); Gradient: Mobile Phase A: Mobile Phase B=20:80 (v/v); HPLC Equipment: HPLC-Agilent, Back pressure: 100 bar; Column temperature: 35° C.

Chiral Prep-HPLC Condition: CHIRALPAK IE 250*20.0 mml, 5 μm; Mobile phase: A for MtBE (0.1%2M NH3 MeOH) (%) and B for MeOH:DCM=50:50; Gradient: Mobile Phase A: Mobile Phase B=20:80 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 67: (1S,3R)-3-(3-((2-(1-((1-(3-(2,6-dioxopiperidin-3-yl)-2-oxo-2,3-dihydrobenzo[d]oxazol-6-yl)piperidin-4-yl)methyl)-4-methylpiperidin-4-yl)-4-fluoro-1,1-dioxido-2,3-dihydrobenzo[d]isothiazol-5-yl)amino)-1H-pyrazol-5-yl)cyclopentyl tert-butylcarbamate

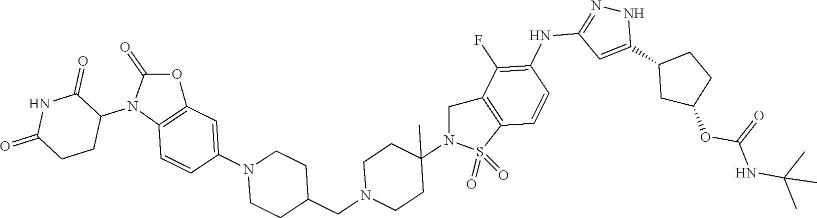 [SMILES: CC(C)(C)NC(=O)O[C@H]1CC[C@@H](c2cc(Nc3ccc4c(c3F)CN(C3(C)CCN(CC5CCN(c6ccc7c(c6)oc(=O)n7C6CCC(=O)NC6=O)CC5)CC3)S4(=O)=O)n[nH]2)C1]

The titled compound was synthesized in the procedures similar to Example 62. ¹H NMR (500 MHz, DMSO) δ12.00 (s, 1H), 11.17 (s, 1H), 8.83 (s, 1H), 8.33 (d, J=8.7 Hz, 1H), 7.58 (d, J=8.6 Hz, 1H), 7.07 (d, J=9.0 Hz, 2H), 6.78 (d, J=9.5 Hz, 2H), 5.83 (s, 1H), 5.32 (dd, J=12.8, 5.1 Hz, 1H), 5.06-4.96 (m, 1H), 4.51 (s, 2H), 3.47 (s, 3H), 3.22 (d, J=10.0 Hz, 2H), 3.10-3.01 (m, 1H), 2.94-2.84 (m, 2H), 2.72-2.64 (m, 4H), 2.45-2.41 (m, 1H), 2.28-2.14 (m, 4H), 2.06-1.96 (m, 2H), 1.94-1.84 (m, 1H), 1.84-1.67 (m, 7H), 1.66-1.54 (m, 2H), 1.40 (s, 3H), 1.34-1.24 (m, 3H), 1.21 (s, 9H). LC-MS (ESI): m/z [M+H]⁺=890.5;

### Example 68: (1S,3R)-3-(3-((2-((4S)-1-((1-(1-(2,6-dioxopiperidin-3-yl)-3-methyl-2-oxo-2,3-dihydro-1H-benzo[d]imidazol-5-yl)piperidin-4-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl tert-butylcarbamate

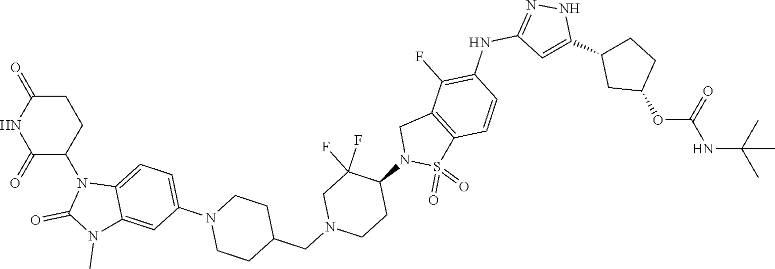 [SMILES: Cn1c(=O)n(C2CCC(=O)NC2=O)c2ccc(N3CCC(CN4CC[C@H](N5Cc6c(ccc(Nc7cc([C@@H]8CC[C@H](OC(=O)NC(C)(C)C)C8)[nH]n7)c6F)S5(=O)=O)C(F)(F)C4)CC3)cc21]

The titled compound was synthesized in the procedures similar to Example 36. ¹H NMR (500 MHz, DMSO) δ 12.00 (s, 1H), 11.06 (s, 1H), 8.82 (s, 1H), 8.33 (s, 1H), 7.58 (d, J=8.7 Hz, 1H), 6.93 (d, J=8.6 Hz, 1H), 6.79 (d, J=30.2 Hz, 2H), 6.64 (d, J=7.0 Hz, 1H), 5.84 (s, 1H), 5.3-5.27 (m, 1H), 4.98 (s, 1H), 4.75 (d, J=14.3 Hz, 1H), 4.54 (d, J=13.1 Hz, 1H), 4.05-3.89 (m, 1H), 3.6-3.58 (m, 2H), 3.31 (s, 3H), 3.19-3.16 (m, 1H), 3.08-3.03 (m, 1H), 2.98-2.85 (m, 2H), 2.73-2.62 (m, 3H), 2.61-2.52 (m, 3H), 2.46-2.45 (m, 1H), 2.38-2.27 (m, 2H), 2.26-2.14 (m, 1H), 2.07-1.95 (m, 2H), 1.95-1.86 (m, 1H), 1.82-1.78 (m, 3H), 1.75-1.55 (m, 4H), 1.27-1.24 (m, 2H), 1.21 (s, 9H). LC-MS (ESI): m/z [M+H]⁺=925.57.

### Example 69: (1R,3S)-3-(3-((2-(1-((1-(4-((R)-2,6-dioxopiperidin-3-yl)-3,5-difluorophenyl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

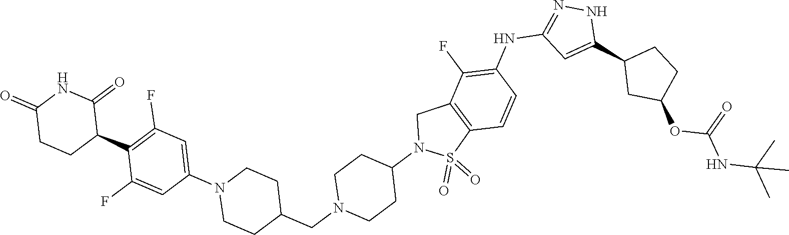 [SMILES: CC(C)(C)NC(=O)O[C@@H]1CC[C@H](c2cc(Nc3ccc4c(c3F)CN(C3CCN(CC5CCN(c6cc(F)c([C@H]7CCC(=O)NC7=O)c(F)c6)CC5)CC3)S4(=O)=O)n[nH]2)C1]

### Step 1: benzyl (1-(tert-butyl)-3-((1S,3R)-3-(((4-nitrophenoxy)carbonyl)oxy)cyclopentyl)-1H-pyrazol-5-yl)carbamate

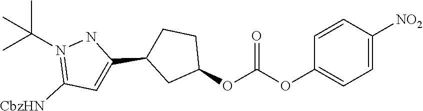

To a solution of benzyl (1-(tert-butyl)-3-((1S,3R)-3-hydroxycyclopentyl)-1H-pyrazol-5-yl)carbamate (107 g, 300 mmol, Vendor Chemscene, Cat #CS-0173525) in DCM (1000 mL) was added pyridine (118.5 g, 1500 mmol), DMAP (3.6 g, 30 mmol), and 4-nitrophenyl carbonochloridate (63 g, 315 mmol), successively. The resulting mixture was stirred at room temperature for 16 h. The reaction mixture was concentrated under reduced pressure, and the residue was purified by silica gel chromatography, eluting with PE/EA (3:1) to afford the product (141 g, 90% yield). LC-MS (ESI): m/z [M+H]⁺=523.2

### Step 2: benzyl (5-((1S,3R)-3-(((4-nitrophenoxy)carbonyl)oxy)cyclopentyl)-1H-pyrazol-3-yl)carbamate

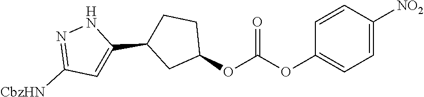 [SMILES: O=C(Nc1cc([C@H]2CC[C@@H](OC(=O)Oc3ccc([N+](=O)[O-])cc3)C2)[nH]n1)OCc1ccccc1]

To a flask was added benzyl (1-(tert-butyl)-3-((1S,3R)-3-(((4-nitrophenoxy)carbonyl) oxy)cyclopentyl)-1H-pyrazol-5-yl)carbamate (141 g, 270 mmol) and formic acid (1200 mL). The mixture was stirred at 80° C. for over night. The solvent was removed under reduced pressure to obtain the crude product. LC-MS (ESI): m/z [M+H]⁺=467.2.

### Step 3: benzyl (5-((1S,3R)-3-((tert-butylcarbamoyl)oxy)cyclopentyl)-1H-pyrazol-3-yl)carbamate

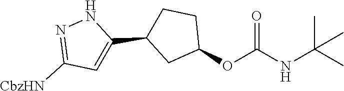 [SMILES: CC(C)(C)NC(=O)O[C@@H]1CC[C@H](c2cc(NC(=O)OCc3ccccc3)n[nH]2)C1]

To a solution of benzyl (5-((1SR,3R)-3-(((4-nitrophenoxy)carbonyl)oxy)cyclopentyl)-1H-pyrazol-3-yl)carbamate (100 g, 215 mmol) in THF (1000 mL) was added tert-butylamine (78.5 g, 1075 mmol) and DIEA (138.7 g, 1075 mmol) at 0° C. The resulting mixture was stirred at room temperature for 3 h. The mixture was filtered, and the filtrate was poured into water. The mixture was extracted by EA (500 mL×3), and the combined organic phase was dried over Na-₂SO₄ and concentrated under reduced pressure. The residue was purified by silica gel chromatography (PE/EA=1:1) to afford the product (68.8 g, 80% yield). LC-MS (ESI): m/z [M+H]⁺=401.4

### Step 4: (1R,3S)-3-(3-amino-1H-pyrazol-5-yl)cyclopentyl tert-butylcarbamate

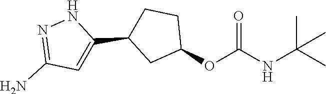 [SMILES: CC(C)(C)NC(=O)O[C@@H]1CC[C@H](c2cc(N)n[nH]2)C1]

To a solution of benzyl (5-((1S,3R)-3-((tert-butylcarbamoyl)oxy)cyclopentyl)-1H-pyrazol-3-yl)carbamate (20 g, 50 mmol) in THF (100 mL) was added Pd/C (10%, wet, 15 g). The suspension was degassed and purged with hydrogen 3 times. The resulting mixture was stirred at room temperature under a hydrogen balloon for 3 h. The mixture was filtered, and the filter cake was washed with EA (50 mL×3) and methanol (50 mL×3). The filtrate was concentrated under reduced pressure to afford the product (11.2 g, 84.2% yield). LC-MS (ESI): m/z [M+H]⁺=267.2

### Step 5: tert-butyl 4-(5-((5-((1S,3R)-3-((tert-butylcarbamoyl)oxy)cyclopentyl)-1H-pyrazol-3-yl)amino)-4-fluoro-1,1-dioxidobenzo[d]isothiazol-2(3H)-yl)piperidine-1-carboxylate

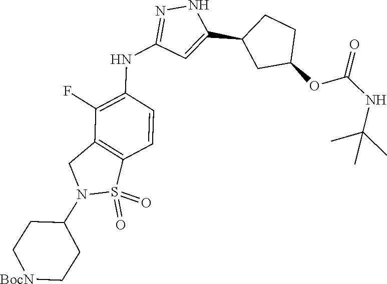 [SMILES: CC(C)(C)NC(=O)O[C@@H]1CC[C@H](c2cc(Nc3ccc4c(c3F)CN(C3CCN(C(=O)OC(C)(C)C)CC3)S4(=O)=O)n[nH]2)C1]

To a mixture of tert-butyl 4-(5-bromo-4-fluoro-1,1-dioxidobenzo[d]isothiazol-2(3H)-yl)piperidine-1-carboxylate (448 mg, 1 mmol) and (1R,3S)-3-(3-amino-1H-pyrazol-5-yl)cyclopentyl tert-butylcarbamate (266 mg, 1 mmol) in 2-methylpropan-2-ol (10 mL) was added BrettPhos Pd G3 (46 mg, 0.05 mmol) and K₂CO₃ (276 mg, 2 mmol). The reaction mixture was stirred under nitrogen atmosphere at 90° C. for 16 h. The mixture was filtered, and the filtrate was concentrated under reduced pressure. The residue was purified by silica gel column chromatography, eluting with DCM/MeOH (10:1) to afford the product (545 mg, 86% yield). LC-MS (ESI): m/z [M+H]⁺=635.3.

### Step 6: (1R,3S)-3-(3-((4-fluoro-1,1-dioxido-2-(piperidin-4-yl)-2,3-dihydrobenzo[d]isothiazol-5-yl)amino)-1H-pyrazol-5-yl)cyclopentyl tert-butylcarbamate

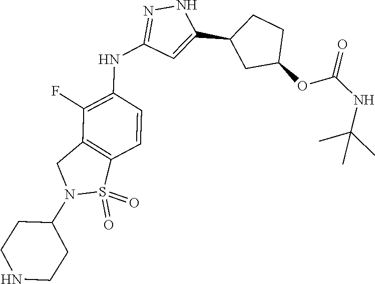 [SMILES: CC(C)(C)NC(=O)O[C@@H]1CC[C@H](c2cc(Nc3ccc4c(c3F)CN(C3CCNCC3)S4(=O)=O)n[nH]2)C1]

To a flask was added tert-butyl 4-(5-((5-((1S,3R)-3-((tert-butylcarbamoyl)oxy) cyclopentyl)-1H-pyrazol-3-yl)amino)-4-fluoro-1,1-dioxidobenzo[d]isothiazol-2(3H)-yl)piperidine-1-carboxylate (545 mg, 0.86 mmol) and 4N HCl in dioxane (5 mL). The resulting solution was stirred at room temperature for 1 h. The mixture was concentrated under reduced pressure to get the crude product without further purification. LC-MS (ESI): m/z [M+H]⁺=535.5.

### Step 7: (1R,3S)-3-(3-((2-(1-((1-(4-((R)-2,6-dioxopiperidin-3-yl)-3,5-difluorophenyl) piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

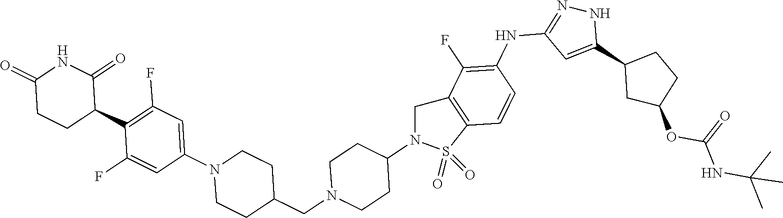 [SMILES: CC(C)(C)NC(=O)O[C@@H]1CC[C@H](c2cc(Nc3ccc4c(c3F)CN(C3CCN(CC5CCN(c6cc(F)c([C@H]7CCC(=O)NC7=O)c(F)c6)CC5)CC3)S4(=O)=O)n[nH]2)C1]

The titled compound was synthesized in the procedures similar to Example 1. ¹H NMR (500 MHz, DMSO) δ 11.99 (s, 1H), 10.87 (s, 1H), 8.78 (s, 1H), 8.32-8.29 (m, 1H), 7.14 (d, J=8.6 Hz, 1H), 6.80-6.75 (m, 1H), 6.67-6.59 (m, 2H), 5.86-5.81 (m, 1H), 5.07-4.98 (m, 1H), 4.53-4.51 (m, 2H), 4.09-3.91 (m, 1H), 3.81-3.76 (m, 2H), 3.44-3.40 (m, 1H), 3.20-3.02 (m, 1H), 2.99-2.81 (m, 2H), 2.79-2.69 (m, 3H), 2.48-2.43 (m, 1H), 2.19-2.14 (m, 2H), 2.13-2.00 (m, 4H), 1.99-1.81 (m, 7H), 1.81-1.66 (m, 5H), 1.63-1.58 (m, 1H), 1.21 (s, 9H), 1.15-1.09 (m, 2H). LC-MS (ESI): m/z [M+H]⁺=855.5

### Example 70: (1R,3S)-3-(3-((2-(1-((1-(4-(2,4-dioxotetrahydropyrimidin-1(2H)-yl)phenyl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

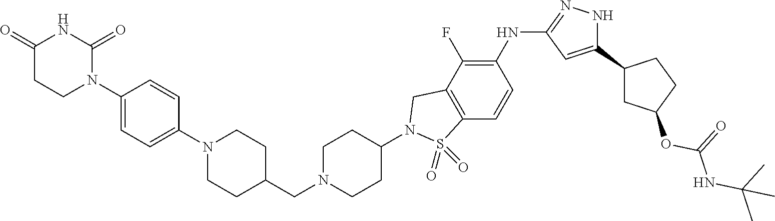 [SMILES: CC(C)(C)NC(=O)O[C@@H]1CC[C@H](c2cc(Nc3ccc4c(c3F)CN(C3CCN(CC5CCN(c6ccc(N7CCC(=O)NC7=O)cc6)CC5)CC3)S4(=O)=O)n[nH]2)C1]

### Step1: (1R,3S)-3-(3-((2-(1-((1-(4-(2,4-dioxotetrahydropyrimidin-1(2H)-yl)phenyl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

(1R,3S)-3-(3-((4-fluoro-1,1-dioxido-2-(piperidin-4-yl)-2,3-dihydrobenzo[d]isothiazol-5-yl)amino)-1H-pyrazol-5-yl)cyclopentyl tert-butylcarbamate (30 mg, 0.056 mmol) and 1-(4-(2,4-dioxotetrahydropyrimidin-1(2H)-yl)phenyl)piperidine-4-carbaldehyde (18.6 mg, 0.061 mmol, Vendor AA BLOCKS LLC, Cat #AA027RIN) were dissolved in N,N-dimethylacetamide (3 mL) and stirred for 15 minutes at room temperature. Sodium triacetoxyborohydride (23.7 mg, 0.11 mmol) was added, and the reaction mixture was stirred for 2 hours at room temperature. The resulting mixture was concentrated, and the residue was purified by column chromatography on silica gel, eluting with DCM/MeOH (10:1) to afford the product (20 mg, 43.4%). The residue 20 mg was purified by prep HPLC (Waters XSelect C18: RD-CO-094 column, eluting with a gradient of acetonitrile/water containing 0.1% FA, 20%-45%) to afford the product (7 mg, 35%).

¹H NMR (500 MHz, DMSO) δ 11.98 (s, 1H), 10.25 (s, 1H), 8.77 (s, 1H), 8.321-8.29 (m, 1H), 7.51 (d, J=8.6 Hz, 1H), 7.15-7.11 (m, 2H), 6.94-6.91 (m, 2H), 6.75 (s, 1H), 5.83-5.82 (m, 1H), 5.02-4.95 (m, 1H), 4.51 (s, 2H), 3.71-3.66 (m, 4H), 3.46-3.40 (m, 1H), 3.09-3.02 (m, 1H), 2.9 (d, J=10.7 Hz, 2H), 2.69-2.63 (m, 4H), 2.48-2.45 (m, 1H), 2.18-2.16 (m, 2H), 2.07-2.00 (m, 3H), 1.94-1.84 (m, 5H), 1.80-1.77 (m, 2H), 1.74-1.70 (m, 2H), 1.68-1.64 (m, 1H), 1.61-1.55 (m, 1H), 1.24-1.22 (m, 1H), 1.21 (s, 9H), 1.18-1.16 (m, 1H). LC-MS (ESI): m/z [M+H]⁺=820.5;

### Example 71: (1R,3S)-3-(3-((2-(1-(1-(4-(2,4-dioxotetrahydropyrimidin-1(2H)-yl)phenyl)piperidine-4-carbonyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

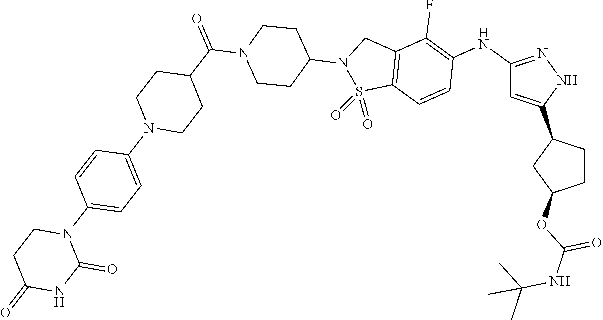 [SMILES: CC(C)(C)NC(=O)O[C@@H]1CC[C@H](c2cc(Nc3ccc4c(c3F)CN(C3CCN(C(=O)C5CCN(c6ccc(N7CCC(=O)NC7=O)cc6)CC5)CC3)S4(=O)=O)n[nH]2)C1]

The titled compound was synthesized in the procedures similar to Example 24. ¹H NMR (500 MHz, DMSO) δ 11.98 (s, 1H), 10.25 (s, 1H), 8.78 (s, 1H), 8.31 (s, 1H), 7.52 (d, J=8.7 Hz, 1H), 7.14 (d, J=9.0 Hz, 2H), 6.94 (d, J=9.1 Hz, 2H), 6.76 (s, 1H), 5.83 (s, 1H), 5.06-4.96 (m, 1H), 4.53 (s, 2H), 4.40 (d, J=13.1 Hz, 1H), 4.08 (d, J=13.2 Hz, 1H), 3.81-3.67 (m, 5H), 3.28-3.20 (m, 1H), 3.10-3.02 (m, 1H), 2.92-2.73 (m, 4H), 2.71-2.62 (m, 3H), 2.47-2.41 (m, 1H), 2.06-1.78 (m, 5H), 1.76-1.64 (m, 7H), 1.62-1.51 (m, 1H), 1.21 (s, 9H). LC-MS (ESI): m/z [M+H]⁺=834.5;

### Example 72: (1R,3S)-3-(3-((2-((S)-1-((1-(4-(2,4-dioxotetrahydropyrimidin-1(2H)-yl)phenyl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl tert-butylcarbamate

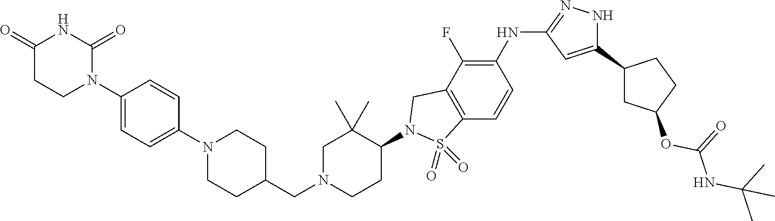 [SMILES: CC(C)(C)NC(=O)O[C@@H]1CC[C@H](c2cc(Nc3ccc4c(c3F)CN([C@H]3CCN(CC5CCN(c6ccc(N7CCC(=O)NC7=O)cc6)CC5)CC3(C)C)S4(=O)=O)n[nH]2)C1]

The titled compound was synthesized in the procedures similar to Example 70. ¹H NMR (500 MHz, DMSO) δ 11.98 (s, 1H), 10.25 (s, 1H), 8.77 (s, 1H), 8.31 (t, J=7.6 Hz, 1H), 7.54 (d, J=8.7 Hz, 1H), 7.13 (d, J=8.9 Hz, 2H), 6.93 (d, J=9.0 Hz, 2H), 6.76 (s, 1H), 5.83 (s, 1H), 5.06-4.96 (m, 1H), 4.54 (s, 2H), 3.69 (t, J=6.7 Hz, 4H), 3.31-3.28 (m, 1H), 3.25-3.20 (m, 1H), 3.11-3.01 (m, 1H), 2.95 (d, J=9.8 Hz, 1H), 2.71-2.66 (m, 3H), 2.57-2.53 (d, J=11.9 Hz, 1H), 2.47-2.43 (m, 1H), 2.25-2.07 (m, 3H), 2.06-1.86 (m, 3H), 1.85-1.77 (m, 3H), 1.75-1.55 (m, 4H), 1.53-1.47 (m, 1H), 1.21 (s, 11H), 1.13 (s, 3H), 0.93 (s, 3H). LC-MS (ESI): m/z [M+H]⁺=848.5;

### Example 73: (1R,3S)-3-(3-((2-((S)-1-((1-(4-(2,4-dioxotetrahydropyrimidin-1(2H)-yl)phenyl)piperidin-4-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl tert-butylcarbamate

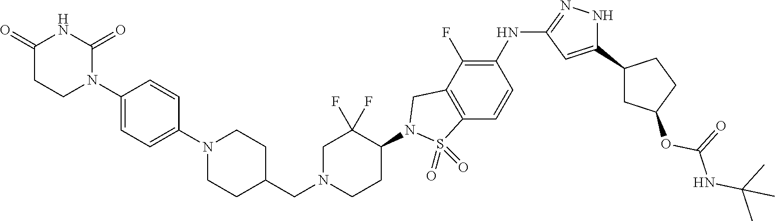 [SMILES: CC(C)(C)NC(=O)O[C@@H]1CC[C@H](c2cc(Nc3ccc4c(c3F)CN([C@H]3CCN(CC5CCN(c6ccc(N7CCC(=O)NC7=O)cc6)CC5)CC3(F)F)S4(=O)=O)n[nH]2)C1]

### Step 1: tert-butyl (S)-4-(5-bromo-4-fluoro-1,1-dioxidobenzo[d]isothiazol-2(3H)-yl)-3,3-difluoropiperidine-1-carboxylate

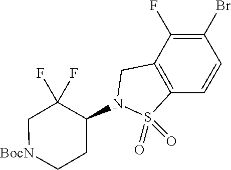 [SMILES: CC(C)(C)OC(=O)N1CC[C@H](N2Cc3c(ccc(Br)c3F)S2(=O)=O)C(F)(F)C1]

The titled compound was synthesised in the procedure similar to example 17 and further separated by chiral HPLC to give Enantiomer 1 (100% ee); Retention time: 4.85 min. LC-MS (ESI): m/z [M+H]⁺=485.4

Chiral analytical method: Column: CHIRAL PAK IH 4.6*100 mm 5 μm; Mobile phase: A for CO2 and B for MeOH; Gradient (B %) 10% to 50% in 3 min, hold 1 min at 50%; 50% to 10% in 0.1 min. Hold 1.9 min at 10%; HPLC Equipment: HPLC-Agilent, Back pressure: 100 bar; Column temperature: 35° C.

Chiral analytical method: Column: CHIRAL PAK IH 20*250.0 mml, 5 μm; Mobile phase: A for CO2 and B for MeOH; Gradient (B %) 10% to 25% in 3 min, hold 4 min at 25%; Flow Rate: 40 mL/min, Wavelength: UV 220 nm, Prep-HPLC Equipment: Prep-SFC-350; Column temperature: 25° C.

### Step 2: (1R,3S)-3-(3-((2-((S)-1-((1-(4-(2,4-dioxotetrahydropyrimidin-1(2H)-yl)phenyl)piperidin-4-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl tert-butylcarbamate

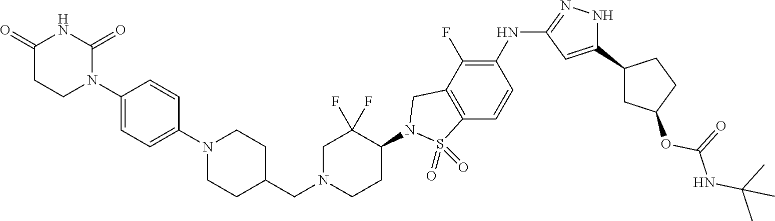 [SMILES: CC(C)(C)NC(=O)O[C@@H]1CC[C@H](c2cc(Nc3ccc4c(c3F)CN([C@H]3CCN(CC5CCN(c6ccc(N7CCC(=O)NC7=O)cc6)CC5)CC3(F)F)S4(=O)=O)n[nH]2)C1]

The titled compound was synthesized in the procedures similar to Example 70. ¹H NMR (500 MHz, DMSO) δ 12.00 (s, 1H), 10.25 (s, 1H), 8.83 (s, 1H), 8.33 (t, J=7.9 Hz, 1H), 7.58 (d, J=8.7 Hz, 1H), 7.13 (d, J=8.9 Hz, 2H), 6.93 (d, J=9.0 Hz, 2H), 6.76 (s, 1H), 5.84 (s, 1H), 5.06-4.96 (m, 1H), 4.75 (d, J=14.4 Hz, 1H), 4.54 (d, J=13.3 Hz, 1H), 3.99 (dd, J=25.3, 12.8 Hz, 1H), 3.69 (t, J=6.7 Hz, 4H), 3.23-3.13 (m, 1H), 3.12-3.02 (m, 1H), 2.94 (d, J=10.2 Hz, 1H), 2.71-2.66 (m, 4H), 2.56-2.54 (m, 1H), 2.46-2.42 (m, 1H), 2.35-2.25 (m, 3H), 2.25-2.15 (m, 1H), 2.08-1.98 (m, 1H), 1.97-1.86 (m, 1H), 1.85-1.65 (m, 6H), 1.62-1.52 (m, 1H), 1.21 (s, 11H). LC-MS (ESI): m/z [M+H]⁺=856.5;

### Example 74: (1R,3S)-3-(3-((2-(1-((1-(4-(2,4-dioxotetrahydropyrimidin-1(2H)-yl)-3-fluoro-5-methylphenyl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

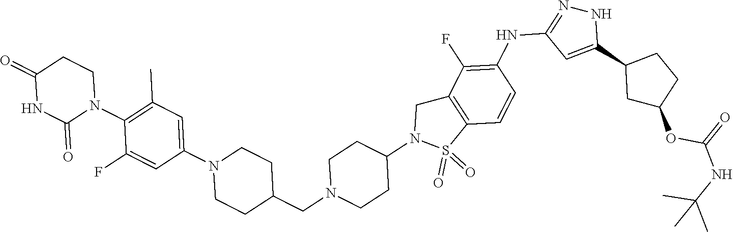 [SMILES: Cc1cc(N2CCC(CN3CCC(N4Cc5c(ccc(Nc6cc([C@H]7CC[C@@H](OC(=O)NC(C)(C)C)C7)[nH]n6)c5F)S4(=O)=O)CC3)CC2)cc(F)c1N1CCC(=O)NC1=O]

### Step 1: 1-(4-(2,4-dioxotetrahydropyrimidin-1(2H)-yl)-3-fluoro-5-methylphenyl) piperidine-4-carbaldehyde

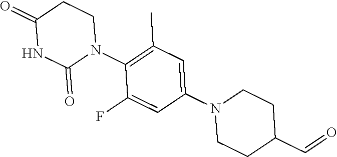 [SMILES: Cc1cc(N2CCC(C=O)CC2)cc(F)c1N1CCC(=O)NC1=O]

The titled compound was synthesized in the procedures similar to Example 22.

### Step 2: (1R,3S)-3-(3-((2-(1-((1-(4-(2,4-dioxotetrahydropyrimidin-1(2H)-yl)-3-fluoro-5-methylphenyl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyltert-butylcarbamate

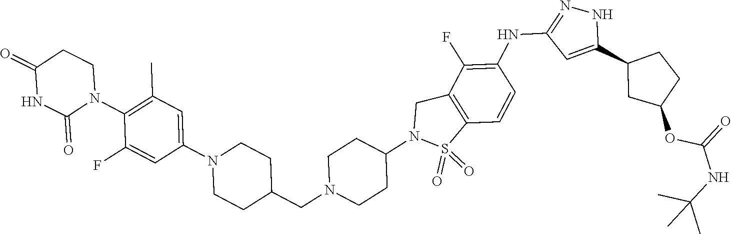 [SMILES: Cc1cc(N2CCC(CN3CCC(N4Cc5c(ccc(Nc6cc([C@H]7CC[C@@H](OC(=O)NC(C)(C)C)C7)[nH]n6)c5F)S4(=O)=O)CC3)CC2)cc(F)c1N1CCC(=O)NC1=O]

The titled compound was synthesized in the procedures similar to Example 70. ¹H NMR (500 MHz, DMSO) δ 12.00 (s, 1H), 10.25 (s, 1H), 8.83 (s, 1H), 8.33 (t, J=7.9 Hz, 1H), 7.58 (d, J=8.7 Hz, 1H), 7.13 (d, J=8.9 Hz, 2H), 6.93 (d, J=9.0 Hz, 2H), 6.76 (s, 1H), 5.84 (s, 1H), 5.06-4.96 (m, 1H), 4.75 (d, J=14.4 Hz, 1H), 4.54 (d, J=13.3 Hz, 1H), 3.99 (dd, J=25.3, 12.8 Hz, 1H), 3.69 (t, J=6.7 Hz, 4H), 3.23-3.13 (m, 1H), 3.12-3.02 (m, 1H), 2.94 (d, J=10.2 Hz, 1H), 2.71-2.66 (m, 4H), 2.56-2.54 (m, 1H), 2.46-2.42 (m, 1H), 2.35-2.25 (m, 3H), 2.25-2.15 (m, 1H), 2.08-1.98 (m, 1H), 1.97-1.86 (m, 1H), 1.85-1.65 (m, 6H), 1.62-1.52 (m, 1H), 1.21 (s, 11H). LC-MS (ESI): m/z [M+H]⁺=856.5; LC-MS (ESI): m/z [M+H]⁺=852.5;

### Example 75a: (1R,3S)-3-(3-((2-(1-(((R)-1-(4-(2,4-dioxotetrahydropyrimidin-1(2H)-yl)phenyl)pyrrolidin-3-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

### Step 1: (S)-1-(4-(3-(hydroxymethyl)pyrrolidin-1-yl)phenyl)-3-((2-(trimethylsilyl)ethoxy)methyl)dihydropyrimidine-2,4(1H,3H)-dione

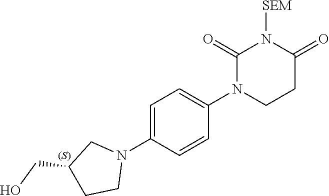

A mixture of 4-(2,4-dioxo-3-((2-(trimethylsilyl)ethoxy)methyl)tetrahydropyrimidin-1(2H)-yl)phenyl trifluoromethanesulfonate (936 mg, 2 mmol), (S)-pyrrolidin-3-ylmethanol (404 mg, 4 mmol), Pd(OAc)2 (90 mg, 0.4 mmol), t-Bu Xphos (340 mg, 0.8 mmol) and Cs2CO3 (1.95 g, 6 mmol) in dioxane (20 mL) was stirred at 100° C. for 16 h under a nitrogen atmosphere. The mixture was concentrated under vacuum and the residue was purified by silica gel chromatography, eluting with EA/PE (100%) to afford the product (450 mg, 53.6% yield). LC-MS (ESI): m/z [M+H]⁺=420.

### Step 2: (S)-1-(4-(3-(hydroxymethyl)pyrrolidin-1-yl)phenyl)dihydropyrimidine-2,4(1H,3H)-dione

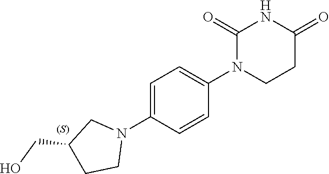

To a solution of (S)-1-(4-(3-(hydroxymethyl)pyrrolidin-1-yl)phenyl)-3-((2-(trimethylsilyl)ethoxy)methyl)dihydropyrimidine-2,4(1H,3H)-dione (450 mg, 1.33 mmol) in DCM (20 mL) was added TFA (4 mL). The mixture was stirred at rt for 3 h. The mixture was concentrated under vacuum and the residue was added NH3 (10 mL, 7 N in MeOH). The mixture was stirred at rt for 1 h. The mixture was concentrated under vacuum and the residue was purified by silica gel chromatography, eluting with MeOH in DCM from 0% to 10% to afford the product (180 mg, 58% yield). LC-MS (ESI): m/z [M+H]⁺=290.

### Step 3: (S)-1-(4-(2,4-dioxotetrahydropyrimidin-1(2H)-yl)phenyl)pyrrolidine-3-carbaldehyde

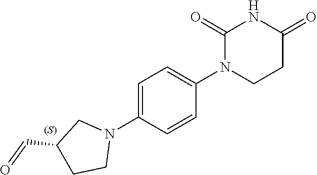

To a solution of (S)-1-(4-(3-(hydroxymethyl)pyrrolidin-1-yl)phenyl)dihydropyrimidine-2,4(1H,3H)-dione (80 mg, 0.28 mmol) in DMSO (5 mL) was added IBX. The mixture was stirred at rt for 16 h. The mixture was diluted with water (20 mL), extracted with EA (3×50 mL). The combined organic layers were washed with aq NaHCO₃ (3×50 mL) and brine (3×50 mL), dried over anhydrous Na2SO4, then filtered and concentrated under reduced pressure to afford the product (50 mg, crude) which was used in the next step directly without further purification. LC-MS (ESI): m/z [M+H]⁺=288.

### Step 4: (1R,3S)-3-(3-((2-(1-(((R)-1-(4-(2,4-dioxotetrahydropyrimidin-1(2H)-yl)phenyl)pyrrolidin-3-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

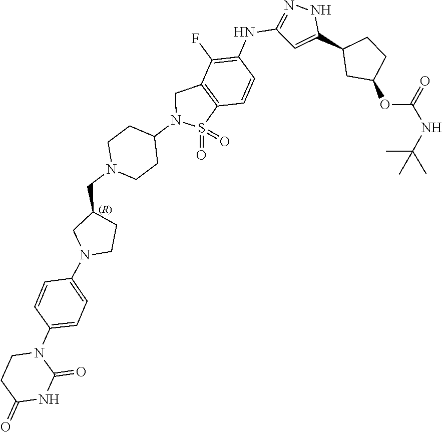

The titled compound was synthesized in the procedures similar to Example 3. ¹H NMR (500 MHz, DMSO-d₆) δ 11.99 (s, 1H), 10.20 (s, 1H), 8.77 (s, 1H), 8.29 (s, 1H), 7.51 (d, J=10 Hz, 1H), 7.09 (t, J=10 Hz, 2H), 6.79-6.72 (m, 1H), 6.51 (d, J=10 Hz, 2H), 5.83 (s, 1H), 5.01-4.94 (m, 1H), 4.53 (s, 2H), 3.66 (t, J=5 Hz, 2H), 3.52-3.43 (m, 3H), 3.41-3.35 (m, 1H), 3.13-2.89 (m, 4H), 2.71-2.65 (m, 2H), 2.65-2.51 (m, 1H), 2.47-2.36 (m, 1H), 2.42-2.35 (m, 2H), 2.17-1.90 (m, 4H), 1.97-1.85 (m, 5H), 1.77-1.68 (m, 3H), 1.65-1.53 (m, 1H), 1.21 (s, 9H). LC-MS (ESI): m/z [M+H]⁺=806.

### Example 75b: (1R,3S)-3-(3-((2-(1-(((S)-1-(4-(2,4-dioxotetrahydropyrimidin-1(2H)-yl)phenyl)pyrrolidin-3-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

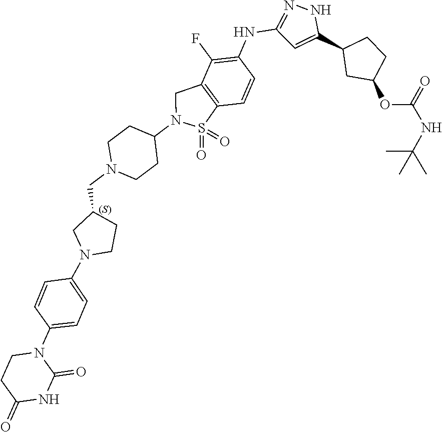

The titled compound was synthesized in the procedures similar to Example 75a. ¹H NMR (500 MHz, DMSO-d₆) δ 11.99 (s, 1H), 10.21 (s, 1H), 8.77 (s, 1H), 8.29 (s, 1H), 7.51 (d, J=10 Hz, 1H), 7.09 (t, J=10 Hz, 2H), 6.79-6.72 (m, 1H), 6.51 (d, J=10 Hz, 2H), 5.83 (s, 1H), 5.01-4.94 (m, 1H), 4.53 (s, 2H), 3.66 (t, J=5 Hz, 2H), 3.54-3.44 (m, 3H), 3.43-3.32 (m, 1H), 3.13-2.89 (m, 4H), 2.71-2.65 (m, 2H), 2.65-2.51 (m, 1H), 2.47-2.36 (m, 1H), 2.42-2.35 (m, 2H), 2.17-1.90 (m, 4H), 1.97-1.85 (m, 5H), 1.77-1.68 (m, 3H), 1.65-1.53 (m, 1H), 1.21 (s, 9H). LC-MS (ESI): m/z [M+H]⁺=806.

### Example 76: (1R,3S)-3-(3-((2-(1-((1-(4-(2,4-dioxotetrahydropyrimidin-1(2H)-yl)-3-methylphenyl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

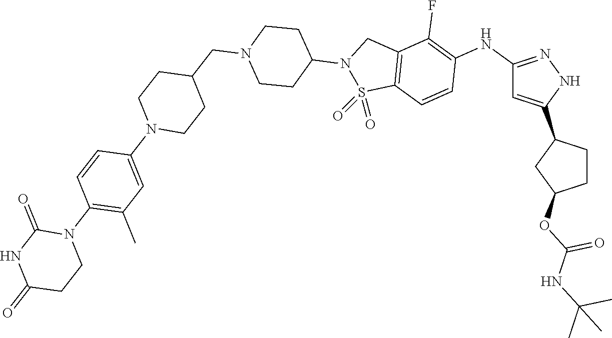 [SMILES: Cc1cc(N2CCC(CN3CCC(N4Cc5c(ccc(Nc6cc([C@H]7CC[C@@H](OC(=O)NC(C)(C)C)C7)[nH]n6)c5F)S4(=O)=O)CC3)CC2)ccc1N1CCC(=O)NC1=O]

### Step 1: 1-(4-(2,4-dioxotetrahydropyrimidin-1(2H)-yl)-3-methylphenyl)piperidine-4-carbaldehyde

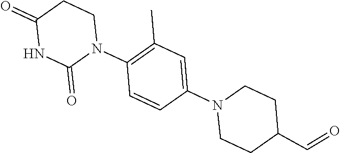 [SMILES: Cc1cc(N2CCC(C=O)CC2)ccc1N1CCC(=O)NC1=O]

The titled compound was synthesized in the procedures similar to Example 74.

### Step 2: (1R,3S)-3-(3-((2-(1-((1-(4-(2,4-dioxotetrahydropyrimidin-1(2H)-yl)-3-methylphenyl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

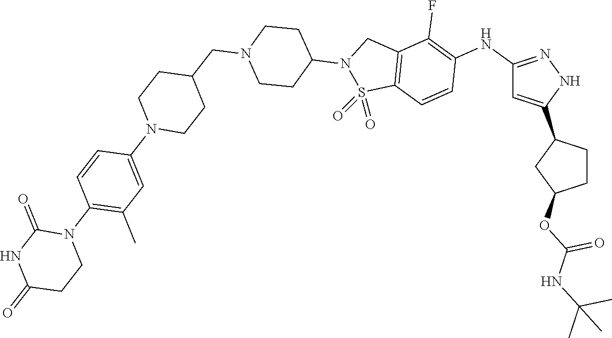 [SMILES: Cc1cc(N2CCC(CN3CCC(N4Cc5c(ccc(Nc6cc([C@H]7CC[C@@H](OC(=O)NC(C)(C)C)C7)[nH]n6)c5F)S4(=O)=O)CC3)CC2)ccc1N1CCC(=O)NC1=O]

The titled compound was synthesized in the procedures similar to Example 70. ¹H NMR (500 MHz, DMSO) δ 12.00 (s, 1H), 10.24 (s, 1H), 8.79 (s, 1H), 8.31 (s, 1H), 7.53 (d, J=8.2 Hz, 1H), 7.05 (d, J=8.6 Hz, 1H), 6.82 (s, 1H), 6.80-6.73 (m, 2H), 5.83 (s, 1H), 5.06-4.96 (m, 1H), 4.54 (s, 2H), 3.78-3.61 (m, 4H), 3.50-3.42 (m, 2H), 3.10-3.05 (m, 2H), 2.74-2.62 (m, 6H), 2.50-2.42 (m, 1H), 2.12 (s, 3H), 2.08-1.98 (m, 3H), 1.97-1.86 (m, 3H), 1.84-1.78 (m, 3H), 1.76-1.66 (m, 3H), 1.64-1.52 (m, 1H), 1.21 (s, 9H). LC-MS (ESI): m/z [M+H]⁺=838.5

### Example 77: (1R,3S)-3-(3-((2-(1-((1-(4-(2,4-dioxotetrahydropyrimidin-1(2H)-yl)-3-fluorophenyl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

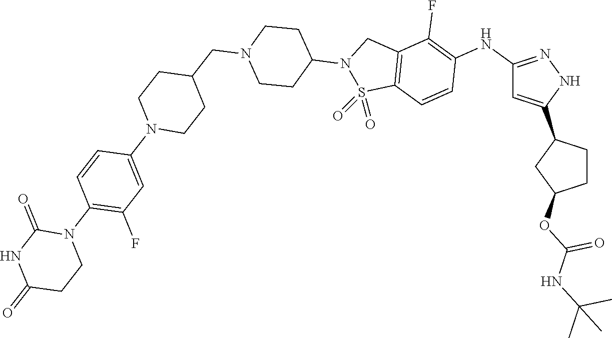 [SMILES: CC(C)(C)NC(=O)O[C@@H]1CC[C@H](c2cc(Nc3ccc4c(c3F)CN(C3CCN(CC5CCN(c6ccc(N7CCC(=O)NC7=O)c(F)c6)CC5)CC3)S4(=O)=O)n[nH]2)C1]

### Step 1: 1-(4-(2,4-dioxotetrahydropyrimidin-1(2H)-yl)-3-fluorophenyl)piperidine-4-carbaldehyde

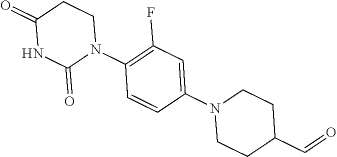 [SMILES: O=CC1CCN(c2ccc(N3CCC(=O)NC3=O)c(F)c2)CC1]

The titled compound was synthesized in the procedures similar to Example 74.

### Step 2: (1R,3S)-3-(3-((2-(1-((1-(4-(2,4-dioxotetrahydropyrimidin-1(2H)-yl)-3-fluorophenyl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyltert-butylcarbamate

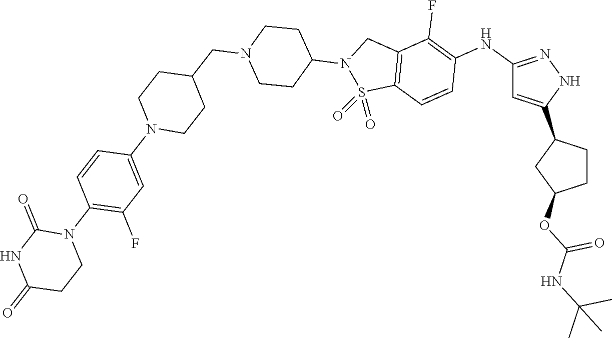 [SMILES: CC(C)(C)NC(=O)O[C@@H]1CC[C@H](c2cc(Nc3ccc4c(c3F)CN(C3CCN(CC5CCN(c6ccc(N7CCC(=O)NC7=O)c(F)c6)CC5)CC3)S4(=O)=O)n[nH]2)C1]

The titled compound was synthesized in the procedures similar to Example 70. ¹H NMR (500 MHz, DMSO) δ 12.01 (s, 1H), 10.38 (s, 1H), 8.83 (d, J=19.9 Hz, 2H), 8.30 (s, 1H), 7.56 (dd, J=11.9, 8.8 Hz, 1H), 7.21 (t, J=9.0 Hz, 1H), 6.86 (d, J=14.4 Hz, 1H), 6.81-6.72 (m, 2H), 5.84 (s, 1H), 5.06-4.96 (m, 1H), 4.54 (s, 2H), 3.78-3.61 (m, 4H), 3.50-3.42 (m, 2H), 3.10-3.05 (m, 2H), 2.74-2.62 (m, 6H), 2.50-2.42 (m, 1H), 2.08-1.98 (m, 3H), 1.97-1.86 (m, 3H), 1.84-1.78 (m, 3H), 1.76-1.66 (m, 3H), 1.64-1.52 (m, 1H), 1.21 (s, 9H). LC-MS (ESI): m/z [M+H]⁺=838.5;

### Example 78: (1R,3S)-3-(3-((2-((1S,4R)-4-(((R)-4-(4-(2,4-dioxotetrahydropyrimidin-1(2H)-yl)phenyl)-2-(methoxymethyl)piperazin-1-yl)methyl)cyclohexyl)-4-fluoro-1,1-dioxido-2,3-dihydrobenzo[d]isothiazol-5-yl)amino)-1H-pyrazol-5-yl)cyclopentyl tert-butylcarbamate

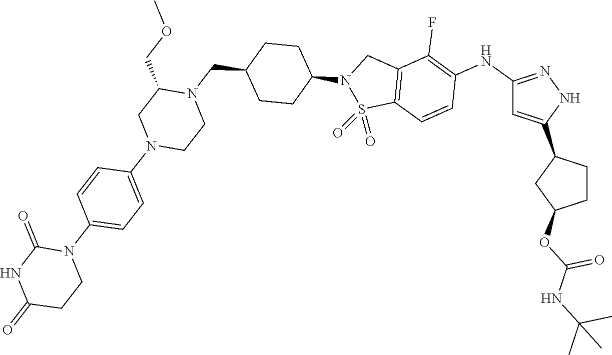 [SMILES: COC[C@H]1CN(c2ccc(N3CCC(=O)NC3=O)cc2)CCN1C[C@H]1CC[C@@H](N2Cc3c(ccc(Nc4cc([C@H]5CC[C@@H](OC(=O)NC(C)(C)C)C5)[nH]n4)c3F)S2(=O)=O)CC1]

### Step 1: tert-butyl (R)-4-(4-(2,4-dioxotetrahydropyrimidin-1(2H)-yl)phenyl)-2-(methoxymethyl)piperazine-1-carboxylate

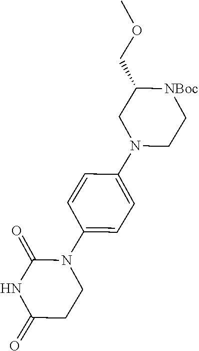 [SMILES: COC[C@H]1CN(c2ccc(N3CCC(=O)NC3=O)cc2)CCN1C(=O)OC(C)(C)C]

To a solution of 1-(4-bromophenyl)dihydropyrimidine-2,4(1H,3H)-dione (300 mg, 1.12 mmol) and tert-butyl (R)-2-(methoxymethyl)piperazine-1-carboxylate (386 mg, 1.67 mmol) in dioxane (20 mL) was added Pd-PEPPSI-IPentCl 2-methylpyridine (94 mg, 0.112 mmol) and Cs₂CO₃ (1.09 g, 3.35 mmol), the reaction mixture was stirred for 16 hours at 100° C. under nitrogen. The resulting mixture was cooled to room temperature, filtered, the filtrate was concentrated under reduce pressure. The residue was purified by silica gel column chromatography, eluting with PE/EA (1:3) to afford the product (220 mg, 47% yield). LC-MS (ESI): m/z [M+H]⁺=419.2.

### Step 2: (R)-1-(4-(3-(methoxymethyl)piperazin-1-yl)phenyl)dihydropyrimidine-2,4(1H,3H)-dione

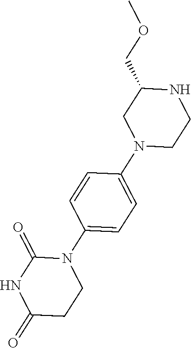 [SMILES: COC[C@H]1CN(c2ccc(N3CCC(=O)NC3=O)cc2)CCN1]

tert-butyl (R)-4-(4-(2,4-dioxotetrahydropyrimidin-1(2H)-yl)phenyl)-2-(methoxymethyl)piperazine-1-carboxylate (100 mg, 0.23 mmol) in DCM (8 mL) was added 4M HCl (2 mL). The reaction mixture was stirred for 2 hours at room temperature. The resulting mixture was concentrated under reduce pressure, dried over in vacuum, to afford the product (76 mg, 100% yield). LC-MS (ESI): m/z [M+H]⁺=319.2.

### Step 3: (1R,3S)-3-(3-((2-((1S,4R)-4-(((R)-4-(4-(2,4-dioxotetrahydropyrimidin-1(2H)-yl)phenyl)-2-(methoxymethyl)piperazin-1-yl)methyl)cyclohexyl)-4-fluoro-1,1-dioxido-2,3-dihydrobenzo[d]isothiazol-5-yl)amino)-1H-pyrazol-5-yl)cyclopentyl tert-butylcarbamate

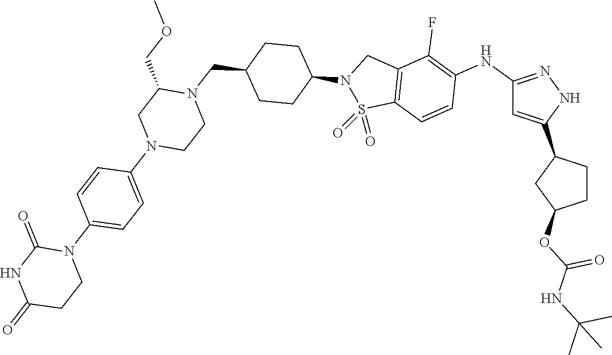 [SMILES: COC[C@H]1CN(c2ccc(N3CCC(=O)NC3=O)cc2)CCN1C[C@H]1CC[C@@H](N2Cc3c(ccc(Nc4cc([C@H]5CC[C@@H](OC(=O)NC(C)(C)C)C5)[nH]n4)c3F)S2(=O)=O)CC1]

The titled compound was synthesized in the procedures similar to Example 18. ¹H NMR (500 MHz, DMSO) δ 11.98 (s, 1H), 10.26 (s, 1H), 8.77 (s, 1H), 8.30 (t, J=10 Hz, 1H), 7.50 (d, J=8.6 Hz, 1H), 7.15 (d, J=8.6 Hz, 2H), 6.91 (d, J=8.9 Hz, 2H), 6.76 (s, 1H), 5.83 (s, 1H), 5.06-4.96 (m, 1H), 4.49 (d, J=14.7 Hz, 2H), 3.69 (t, J=10 Hz, 2H), 3.57-3.54 (m, 3H), 3.09-3.02 (m, 2H), 2.99-2.81 (m, 6H), 2.75-2.66 (m, 5H), 2.45-2.43 (m, 1H), 2.20-2.13 (m, 1H), 2.06-1.88 (m, 5H), 1.77-1.63 (m, 6H), 1.62-1.50 (m, 6H), 1.21 (s, 9H). LC-MS (ESI): m/z [M+H]⁺=864.5;

### Example 79: (1R,3S)-3-(3-((2-((1R,4R)-4-(((S)-4-(4-(2,4-dioxotetrahydropyrimidin-1(2H)-yl)phenyl)-2-(methoxymethyl)piperazin-1-yl)methyl)cyclohexyl)-4-fluoro-1,1-dioxido-2,3-dihydrobenzo[d]isothiazol-5-yl)amino)-1H-pyrazol-5-yl)cyclopentyl tert-butylcarbamate

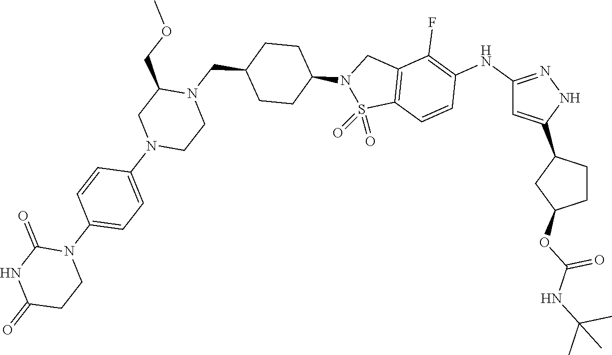 [SMILES: COC[C@@H]1CN(c2ccc(N3CCC(=O)NC3=O)cc2)CCN1C[C@H]1CC[C@@H](N2Cc3c(ccc(Nc4cc([C@H]5CC[C@@H](OC(=O)NC(C)(C)C)C5)[nH]n4)c3F)S2(=O)=O)CC1]

The titled compound was synthesized in the procedures similar to Example 77. ¹H NMR (500 MHz, DMSO) δ 11.98 (s, 1H), 10.26 (s, 1H), 8.77 (s, 1H), 8.30 (t, J=10 Hz, 1H), 7.50 (d, J=8.6 Hz, 1H), 7.15 (d, J=8.6 Hz, 2H), 6.91 (d, J=8.9 Hz, 2H), 6.76 (s, 1H), 5.83 (s, 1H), 5.06-4.96 (m, 1H), 4.49 (d, J=14.7 Hz, 2H), 3.69 (t, J=10 Hz, 2H), 3.57-3.54 (m, 3H), 3.09-3.02 (m, 2H), 2.99-2.81 (m, 6H), 2.75-2.66 (m, 5H), 2.45-2.43 (m, 1H), 2.20-2.13 (m, 1H), 2.06-1.88 (m, 5H), 1.77-1.63 (m, 6H), 1.62-1.50 (m, 6H), 1.21 (s, 9H). LC-MS (ESI): m/z [M+H]⁺=864.5;

### Example 80: (1R,3S)-3-(3-((2-((1S,4S)-4-(((S)-4-(4-(2,4-dioxotetrahydropyrimidin-1(2H)-yl)phenyl)-2-(methoxymethyl)piperazin-1-yl)methyl)cyclohexyl)-4-fluoro-1,1-dioxido-2,3-dihydrobenzo[d]isothiazol-5-yl)amino)-1H-pyrazol-5-yl)cyclopentyl tert-butylcarbamate

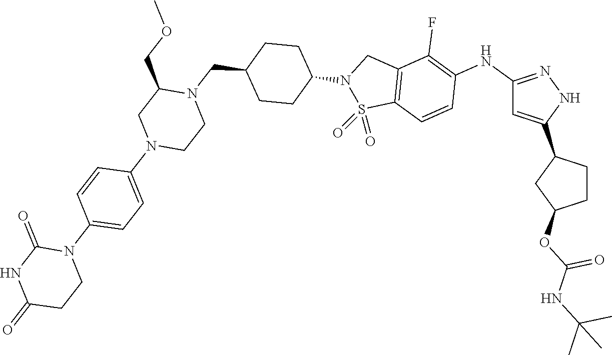 [SMILES: COC[C@@H]1CN(c2ccc(N3CCC(=O)NC3=O)cc2)CCN1C[C@H]1CC[C@H](N2Cc3c(ccc(Nc4cc([C@H]5CC[C@@H](OC(=O)NC(C)(C)C)C5)[nH]n4)c3F)S2(=O)=O)CC1]

The titled compound was synthesized in the procedures similar to Example 78. H NMR (500 MHz, DMSO) δ 11.98 (s, 1H), 10.26 (s, 1H), 8.76 (s, 1H), 8.30 (s, 1H), 7.50 (d, J=8.5 Hz, 1H), 7.15 (d, J=8.7 Hz, 2H), 6.91 (d, J=8.7 Hz, 2H), 6.76 (s, 1H), 5.83 (s, 1H), 5.06-4.96 (m, 1H), 4.49 (d, J=14.7 Hz, 2H), 3.72-3.64 (m, 3H), 3.57-3.54 (m, 3H), 3.09-3.02 (m, 2H), 2.99-2.81 (m, 6H), 2.75-2.66 (m, 5H), 2.45-2.43 (m, 1H), 2.20-2.13 (m, 1H), 2.06-1.88 (m, 5H), 1.77-1.63 (m, 6H), 1.62-1.50 (m, 5H), 1.21 (s, 9H). LC-MS (ESI): m/z [M+H]⁺=864.5;

### Example 81: (1R,3S)-3-(3-((2-(1-((2-(4-(2,4-dioxotetrahydropyrimidin-1(2H)-yl)phen-yl)-2-azaspiro[3.3]heptan-6-yl)methyl)piperidin-4-yl)-4-fluoro-1,1-dioxido-2,3-dihydrobenz-o[d]isothiazol-5-yl)amino)-1H-pyrazol-5-yl)cyclopentyl tert-butylcarbamate

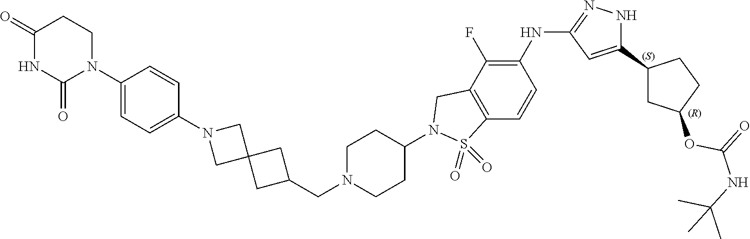

### Step 1: 1-(4-(6-((benzyloxy)methyl)-2-azaspiro[3.3]heptan-2-yl)phenyl)-3-((2-(trimethylsilyl)ethoxy)methyl)dihydropyrimidine-2,4(1H,3H)-dione

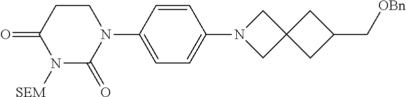 [SMILES: C[Si](C)(C)CCOCN1C(=O)CCN(c2ccc(N3CC4(CC(COCc5ccccc5)C4)C3)cc2)C1=O]

To a mixture of 4-(2,4-dioxo-3-((2-(trimethylsilyl)ethoxy)methyl)tetrahydropyrimidin-1(2H)-yl)phenyl trifluoromethanesulfonate (2 g, 4.27 mmol) and 6-((benzyloxy)methyl)-2-azaspiro[3.3]heptane (1.39 g, 6.41 mmol) in andydrous 1,4-dioxane (10 mL) was added Pd₂(dba)₃ (391.7 mg, 0.427 mmol), RuPhos (398.5 mg, 0.854 mmol) and Cs₂CO₃ (4.17 g, 12.81 mmol). The reaction mixture was stirred under nitrogen atmosphere at 100° C. for 16 h. The mixture was filtered, and the filtrate was concentrated under reduced pressure. The residue was purified by silica gel column chromatography, eluting with PE/EA (1:9) to afford the product (1.6 g, 70% yield). LC-MS (ESI): m/z [M+H]⁺=536.2.

### Step 2: 1-(4-(6-((benzyloxy)methyl)-2-azaspiro[3.3]heptan-2-yl)phenyl)dihydropyrimid-ine-2,4(1H,3H)-dione

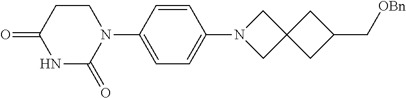 [SMILES: O=C1CCN(c2ccc(N3CC4(CC(COCc5ccccc5)C4)C3)cc2)C(=O)N1]

To a solution of 1-(4-(6-((benzyloxy)methyl)-2-azaspiro[3.3]heptan-2-yl)phenyl)-3-((2-(trimethylsilyl)ethoxy)methyl)dihydropyrimidine-2,4(1H,3H)-dione (1.6 g, 2.99 mmol) in trifluoroacetic acid (15 mL). The reaction mixture was stirred for 1 hours at room temperature. The resulting mixture was concentrated, then the reaction mixture in THF (5 ml) was added NH₃—H₂O (28-30 wt %, 1 mL) dropwise. The reaction mixture was stirred for 10 min at room temperature. The mixture was concentrated under reduced pressure. The residue was purified by silica gel column chromatography, eluting with DCM/MeOH (10:1) to afford the product (900 mg, 75% yield). LC-MS (ESI): m/z [M+H]⁺=406.2.

### Step 3: 1-(4-(6-(hydroxymethyl)-2-azaspiro[3.3]heptan-2-yl)phenyl)dihydropyrimidine-2,4(1H,3H)-dione

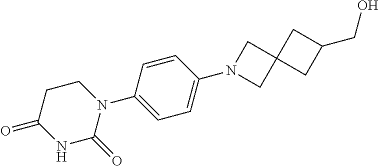 [SMILES: O=C1CCN(c2ccc(N3CC4(CC(CO)C4)C3)cc2)C(=O)N1]

To a solution of 1-(4-(3-(hydroxymethyl)azetidin-1-yl)phenyl)-3-((2-(trimeth-ylsilyl)e-thoxy)meth-yl)dihydropyrimidine-2,4(1H,3H)-dione (200 mg, 0.49 mmol) in THF (15 mL) was added Pd/C (10%, wet, 300 mg). The suspension was degassed and purged with hydrogen 3 times. The resulting mixture was stirred at room temperature under a hydrogen balloon for 3 h. The mixture was filtered, and the filter cake was washed with EA (50 mL□3). The filtrate was concentrated under reduced pressure to afford the product (600 mg, 88% yield). LC-MS (ESI): m/z [M+H]⁺=316.3.

### Step 4: 2-(4-(2,4-dioxotetrahydropyrimidin-1(2H)-yl)phenyl)-2-azaspiro[3.3]heptane-6-carbaldehyde

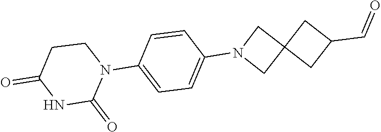 [SMILES: O=CC1CC2(C1)CN(c1ccc(N3CCC(=O)NC3=O)cc1)C2]

To a stirred solution of 1-(4-(6-(hydroxymethyl)-2-azaspiro[3.3]heptan-2-yl)phenyl)di-hydropyrimidine-2,4(1H,3H)-dione (200 mg, 0.64 mmol) in DMSO (5 mL) was added IBX (536 mg, 1.26 mmol). The resulting solution was stirred at room temperature for 2 h. quenched with NaHCO₃ (2 mL) The reaction mixture was poured into water (20 ml) and extracted with EA (50 mL×3). The combined organic layers were dried over anhydrous sodium sulfate, filtrated, and concentrated under reduced pressure to afford the crude product (120 mg, 60% yield). LC-MS (ESI): m/z [M+H]⁺=313.2.

### Step 5: (1S,3R)-3-(3-((2-(1-((1-(4-(2,4-dioxotetrahydropyrimidin-1(2H)-yl)phenyl)a-zetidin-3-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

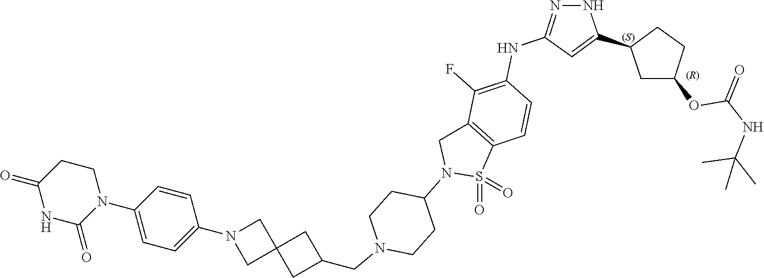

To a solution of (1R,3S)-3-(3-((4-fluoro-1,1-dioxido-2-(piperidin-4-yl)-2,3-dihydrobe-nzo[d]isothiazol-5-yl)amino)-1H-pyrazol-5-yl)cyclopentyl tert-butyl carbamate (30 mg, 0.056 mmol) and 2-(4-(2,4-dioxotetrahydropyrimidin-1(2H)-yl)phenyl)-2-azaspiro[3.3]heptane-6-carbaldehyde (26 mg, 0.084 mmol) in DMA (1.5 mL) was stirred for 30 min, then STAB (24 mg, 0.112 mmol) was added. the reaction mixture was stirred for 16 hours at room tempe-rature. The resulting mixture was concentrated under reduce pressure, the residue was purified by column chromatography on silica gel, eluting with DCM/MeOH (10:1) to afford the product (20 mg, 46%). The residue 20 mg was purified by prep HPLC (Waters XSelect C18: RD-CO-094 column, eluting with a gradient of acetonitrile/water containing 0.1% FA, 20%-40%) to afford the product (10 mg, 22%). LC-MS (ESI): m/z [M+H]⁺=832.51.

¹H NMR (500 MHz, DMSO) δ 11.99 (s, 1H), 10.22 (s, 1H), 8.77 (s, 1H), 8.28 (s, 1H), 7.50 (d, J=8.6 Hz, 1H), 7.08 (d, J=8.7 Hz, 2H), 6.76 (s, 1H), 6.38 (d, J=8.7 Hz, 2H), 5.83 (s, 1H), 4.98 (s, 1H), 4.50 (s, 2H), 3.82 (s, 2H), 3.69 (s, 2H), 3.66 (t, J=6.7 Hz, 2H), 3.44-3.37 (m, 2H), 3.12-3.01 (m, 1H), 2.86 (d, J=11.2 Hz, 2H), 2.67 (t, J=6.7 Hz, 2H), 2.38-2.26 (m, 5H), 1.94-1.81 (m, 3H), 1.94-1.81 (m, 7H), 1.75-1.68 (m, 2H), 1.58 (s, 1H), 1.21 (s, 9H).

### Example 82: (1R,3S)-3-(3-((2-(1-(2-(4-(2,4-dioxotetrahydropyrimidin-1(2H)-yl)phenyl)-2-azaspiro[3.3]heptan-6-yl)piperidin-4-yl)-4-fluoro-1,1-dioxido-2,3-dihydrobenzo[d]iso-thiazol-5-yl)amino)-1H-pyrazol-5-yl)cyclopentyl tert-butylcarbamate

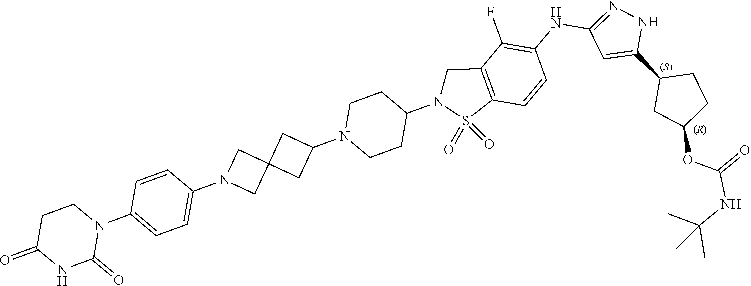

### Step 1: 1-(4-(6,6-dimethoxy-2-azaspiro[3.3]heptan-2-yl)phenyl)-3-((2-(trimethylsilyl)e-thoxy)methyl)dihydropyrimidine-2,4(1H,3H)-dione

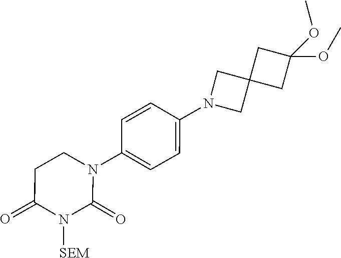 [SMILES: COC1(OC)CC2(CN(c3ccc(N4CCC(=O)N(COCC[Si](C)(C)C)C4=O)cc3)C2)C1]

To a mixture of 4-(2,4-dioxo-3-((2-(trimethylsilyl)ethoxy)methyl)tetrahydropyrimidin-1(2H)-yl)phenyl trifluoromethanesulfonate (1 g, 2.13 mmol) and 6,6-dimethoxy-2-azaspiro[3.3]heptane, oxalic acid (791 mg, 3.20 mmol) in andydrous 1,4-dioxane (10 mL) was added Pd₂(dba)₃ (196 mg, 0.313 mmol), RuPhos (196 mg, 0.417 mmol) and Cs₂CO₃ (2.13 g, 6.4 mmol). The reaction mixture was stirred under nitrogen atmosphere at 100° C. for 16 h. The mixture was filtered, and the filtrate was concentrated under reduced pressure. The residue was purified by silica gel column chromatography, eluting with PE/EA (1:9) to afford the product (800 mg, 70% yield). LC-MS (ESI): m/z [M+H]⁺=476.2.

### Step 2: 1-(4-(6-oxo-2-azaspiro[3.3]heptan-2-yl)phenyl)dihydropyrimidine-2,4(1H,3H)-dione

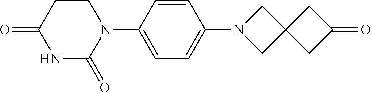 [SMILES: O=C1CC2(C1)CN(c1ccc(N3CCC(=O)NC3=O)cc1)C2]

To a flask was added 1-(4-(6-((benzyloxy)methyl)-2-azaspiro[3.3]heptan-2-yl)phenyl)-3-((2-(trimethylsilyl)ethoxy)methyl)dihydropyrimidine-2,4(1H,3H)-dione (800 mg, 1.68 mmol) and formic acid (15 mL). The reaction mixture was stirred for 16 hours at room temperature. The resulting mixture was concentrated, then the reaction mixture in THF (5 ml) was added NH₃—H₂O (28-30 wt %, 1 mL) dropwise. The reaction mixture was stirred for 10 min at room temperature. The mixture was concentrated under reduced pressure. The residue was purified by silica gel column chromatography, eluting with DCM/MeOH (10:1) to afford the product (400 mg, 80% yield). LC-MS (ESI): m/z [M+H]⁺=300.2.

### Step 3: (1R,3S)-3-(3-((2-(1-(2-(4-(2,4-dioxotetrahydropyrimidin-1(2H)-yl)phenyl)-2-azaspiro[3.3]heptan-6-yl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

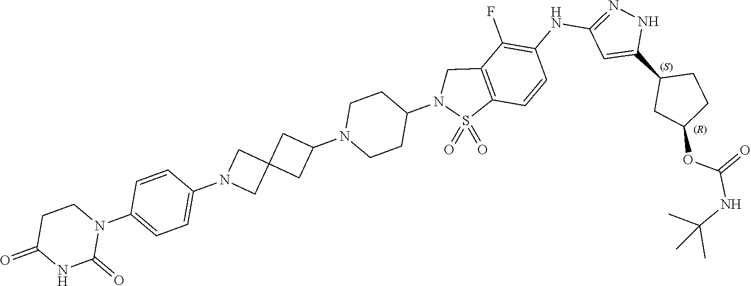

To a solution of (1R,3S)-3-(3-((4-fluoro-1,1-dioxido-2-(piperidin-4-yl)-2,3-dihydrobenz-o[d]isothiazol-5-yl)amino)-1H-pyrazol-5-yl)cyclopentyl tert-butyl carbamate (30 mg, 0.056 mmol) and 2-(4-(2,4-dioxotetrahydropyrimidin-1(2H)-yl)phenyl)-2-azaspiro[3.3]heptane-6-carbaldehyde (25 mg, 0.084 mmol) in DMA (1.5 mL) was stirred for 30 min, then STAB (24 mg, 0.112 mmol) was added. the reaction mixture was stirred for 16 hours at room tempe-rature. The resulting mixture was concentrated under reduce pressure, the residue was purified by column chromatography on silica gel, eluting with DCM/MeOH (10:1) to afford the product (20 mg, 46%). The residue 20 mg was purified by prep HPLC (Waters XSelect C18: RD-CO-094 column, eluting with a gradient of acetonitrile/water containing 0.1% FA, 20%-40%) to afford the product (10 mg, 22%). LC-MS (ESI): m/z [M+H]⁺=818.52.

¹H NMR (500 MHz, DMSO) δ 12.00 (s, 1H), 10.22 (s, 1H), 8.77 (s, 1H), 8.31 (t, J=7.9 Hz, 1H), 7.51 (d, J=8.6 Hz, 1H), 7.09 (d, J=8.5 Hz, 2H), 6.76 (s, 1H), 6.39 (d, J=8.6 Hz, 2H), 5.83 (s, 1H), 4.98 (s, 1H), 4.50 (s, 2H), 3.82 (s, 2H), 3.70 (s, 2H), 3.66 (t, J=6.7 Hz, 2H), 3.50-3.3 (m, 3H), 3.11-3.01 (m, 1H), 2.83 (d, J=8.7 Hz, 2H), 2.70-2.66 (m, 4H), 2.34-2.26 (m, 2H), 2.05-1.95 (m, 3H), 1.94-1.77 (m, 7H), 1.76-1.65 (m, 2H), 1.58 (s, 1H), 1.21 (s, 9H).

### Example 83: (1R,3S)-3-(3-((2-(1-((1-(3-cyclopropyl-4-(2,4-dioxotetrahydropyrimidin-1(2H)-yl)phenyl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

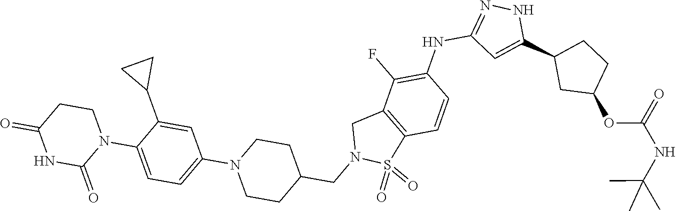 [SMILES: CC(C)(C)NC(=O)O[C@@H]1CC[C@H](c2cc(Nc3ccc4c(c3F)CN(CC3CCN(c5ccc(N6CCC(=O)NC6=O)c(C6CC6)c5)CC3)S4(=O)=O)n[nH]2)C1]

### Step 1: 1-(3-cyclopropyl-4-(2,4-dioxotetrahydropyrimidin-1(2H)-yl)phenyl)piperidine-4-carbaldehyde

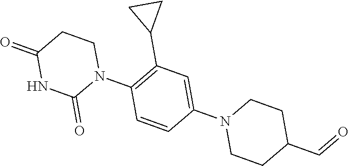 [SMILES: O=CC1CCN(c2ccc(N3CCC(=O)NC3=O)c(C3CC3)c2)CC1]

The titled compound was synthesized in the procedures similar to Example 74.

### Step 2: (1R,3S)-3-(3-((2-(1-((1-(3-cyclopropyl-4-(2,4-dioxotetrahydropyrimidin-1(2H)-yl)phenyl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

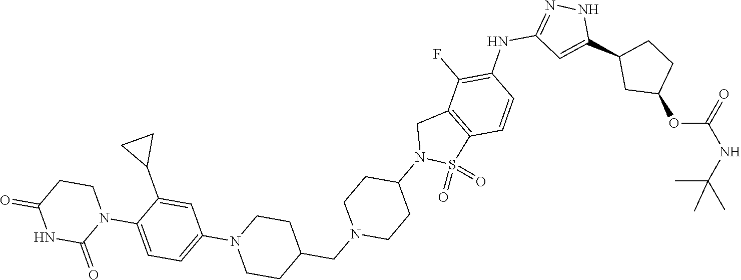 [SMILES: CC(C)(C)NC(=O)O[C@@H]1CC[C@H](c2cc(Nc3ccc4c(c3F)CN(C3CCN(CC5CCN(c6ccc(N7CCC(=O)NC7=O)c(C7CC7)c6)CC5)CC3)S4(=O)=O)n[nH]2)C1]

The titled compound was synthesized in the procedures similar to Example 70. ¹H NMR (500 MHz, DMSO) δ 11.98 (s, 1H), 10.24 (s, 1H), 8.77 (s, 1H), 8.28 (s, 1H), 7.51 (d, J=8.7 Hz, 1H), 7.03 (d, J=8.6 Hz, 1H), 6.76-6.74 (m, 2H), 6.45 (d, J=2.5 Hz, 1H), 5.83 (s, 1H), 4.98 (s, 1H), 4.52 (s, 2H), 3.67-3.64 (m, 3H), 3.55-3.51 (m, 1H), 3.45-3.40 (m, 1H), 3.07-3.02 (m, 1H), 2.91-2.89 (m, 2H), 2.79-2.73 (m, 1H), 2.70-2.59 (m, 3H), 2.54 (s, 2H), 2.46-2.44 (m, 1H), 2.18-2.17 (m, 2H), 2.05-2.03 (m, 3H), 1.91-1.82 (m, 6H), 1.79-1.58 (m, 6H), 1.28-1.12 (m, 9H), 0.91-0.88 (m, 1H), 0.86-0.73 (m, 2H), 0.55-0.45 (m, 1H). LC-MS (ESI): m/z [M+H]⁺=860.5.

### Example 84: (1R,3S)-3-(3-((2-(1-(4-(4-(2,4-dioxotetrahydropyrimidin-1(2H)-yl)phenoxy)cyclohexyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

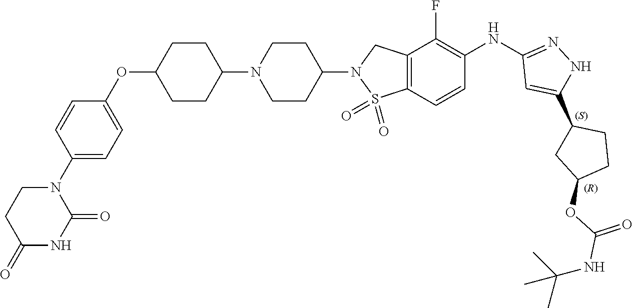

### Step 1: 1-(4-((1,4-dioxaspiro[4.5]decan-8-yl)oxy)phenyl)-3-((2-(trimethylsilyl)ethox-y)methyl)dihydropyrimidine-2,4(1H,3H)-dione

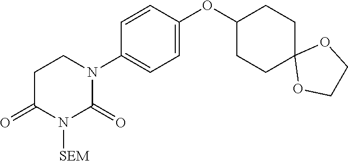 [SMILES: C[Si](C)(C)CCOCN1C(=O)CCN(c2ccc(OC3CCC4(CC3)OCCO4)cc2)C1=O]

A solution of 1-(4-hydroxyphenyl)-3-((2-(trimethylsilyl)ethoxy)methyl)dihydrop-yrimidine-2,4(1H,3H)-dione (1 g, 2.97 mmol), 1,4-dioxaspir-o[4.5]decan-8-ol (564 mg, 3.57 mmol) and PPh₃ (1.16 g, 4.45 mmol) in THF (20 mL) was added DIAD (899 mg, 4.45 mmol) in THF (4 mL) dropwise at 0° C. under nitrogen atmosphere. The reaction mixture was stirred under nitrogen atmosphere at 0° C. for 16 h. The resulting mixture was concentrated under reduced pressure, and the residue was purified by silica gel chromatography (PE/EA=2:1) to afford the product (1 g, 71% yield). LC-MS (ESI): m/z [M+H]⁺=477.3.

### Step 2: 1-(4-((1,4-dioxaspiro[4.5]decan-8-yl)oxy)phenyl)dihydropyrimidine-2,4(1H,3H)-dione

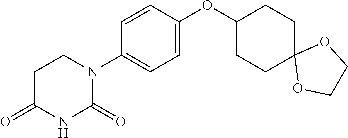 [SMILES: O=C1CCN(c2ccc(OC3CCC4(CC3)OCCO4)cc2)C(=O)N1]

To a solution of 1-(4-((1,4-dioxaspiro[4.5]decan-8-yl)oxy)phenyl)-3-((2-(trimethylsily-1)ethox-y)methyl)dihydropyrimidine-2,4(1H,3H)-dione (1 g, 2.10 mmol) in TFA (15 mL). The reaction mixture was stirred for 1 hours at room temperature. The resulting mixture was concentrated, then the reaction mixture in THF (5 ml) was added NH3-H2O (28-30 wt %, 1 mL) dropwise. The reaction mixture was stirred for 10 min at room temperature. The mixture was concentrated under reduced pressure to afford the product (700 mg, 96% yield). LC-MS (ESI): m/z [M+H]⁺=347.2.

### Step 3: 1-(4-((4-oxocyclohexyl)oxy)phenyl)dihydropyrimidine-2,4(1H,3H)-dione

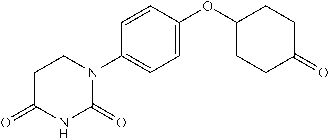 [SMILES: O=C1CCC(Oc2ccc(N3CCC(=O)NC3=O)cc2)CC1]

To a solution of 1-(4-((1,4-dioxaspiro[4.5]decan-8-yl)oxy)phenyl)dihydropyrimidine-2,4(1H,3H)-dione (1 g, 2.10 mmol) in HCl (15 ml, 8M). The reaction mixture was stirred for 2 hours at room temperature. The resulting mixture was concentrated, then the reaction mixture in THF (5 ml) was added NH3-H2O (28-30 wt %, 1 mL) dropwise. The reaction mixture was stirred for 10 min at room temperature. The mixture was concentrated under reduced pressure to afford the product (700 mg, 96% yield). The reaction mixture was quenched and basified to pH=8 with sat. aq NaHCO₃ at 0° C. The layers separated and the aqueous layer was extracted with EA (100 mL×3). The combined organic layers were washed with brine (50 mL) and dried over anhydrous Na2SO4. After filtration, the filtrate was concentrated under reduced pressure. LC-MS (ESI): m/z [M+H]⁺=303.1.

### Step 4: (1R,3S)-3-(3-((2-(1-(4-(4-(2,4-dioxotetrahydropyrimidin-1(2H)-yl)phenoxy)c-yclohexyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

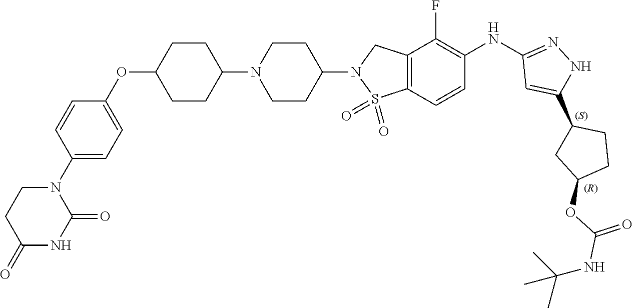

To a solution of (1R,3S)-3-(3-((4-fluoro-1,1-dioxido-2-(piperidin-4-yl)-2,3-dihydrobenz-o[d]isothiazol-5-yl)amino)-1H-pyrazol-5-yl)cyclopentyl tert-butyl carbamate (30 mg, 0.056 mmol) and 2-(4-(2,4-dioxotetrahydropyrimidin-1(2H)-yl)phenyl)-2-azaspiro[3.3]heptane-6-carbaldehyde (25 mg, 0.084 mmol) in DMA (1.5 mL) was stirred for 30 min, then STAB (24 mg, 0.112 mmol) was added. the reaction mixture was stirred for 16 hours at room tempe-rature. The resulting mixture was concentrated under reduce pressure, the residue was purified by column chromatography on silica gel, eluting with DCM/MeOH (10:1) to afford the product (20 mg, 46%). The residue 20 mg was purified by prep HPLC (Waters XSelect C18: RD-CO-094 column, eluting with a gradient of acetonitrile/water containing 0.1% FA, 20%-40%) to afford the product (10 mg, 22%). LC-MS (ESI): m/z [M+H]⁺=821.51.

¹H NMR (500 MHz, DMSO) δ 12.01 (s, 1H), 10.30 (t, J=3.7 Hz, 1H), 8.84 (d, J=22.3 Hz, 1H), 8.30 (d, J=7.5 Hz, 1H), 7.55 (t, J=9.6 Hz, 1H), 7.28-7.17 (m, 2H), 7.05-6.92 (m, 2H), 6.75 (s, 1H), 5.84 (s, 1H), 4.98 (s, 1H), 4.56 (s, 2H), 3.92-3.92 (m, 1H), 3.77-3.68 (m, 2H), 3.62-3.16 (m, 5H), 3.06 (p, J=8.7 Hz, 1H), 2.74-2.66 (m, 2H), 2.49-2.40 (m, 2H), 2.32-1.98 (m, 8H), 1.96-1.86 (m, 2H), 1.83-1.54 (m, 6H), 1.52-1.38 (m, 1H), 1.21 (s, 9H).

### Example 85: (1R,3S)-3-(3-((2-((1S,4R)-4-(((R)-4-(4-(2,4-dioxotetrahydropyrimidin-1(2H)-yl)phenyl)-3-(methoxymethyl)piperazin-1-yl)methyl)cyclohexyl)-4-fluoro-1,1-dioxido-2,3-dihydrobenzo[d]isothiazol-5-yl)amino)-1H-pyrazol-5-yl)cyclopentyl tert-butylcarbamate

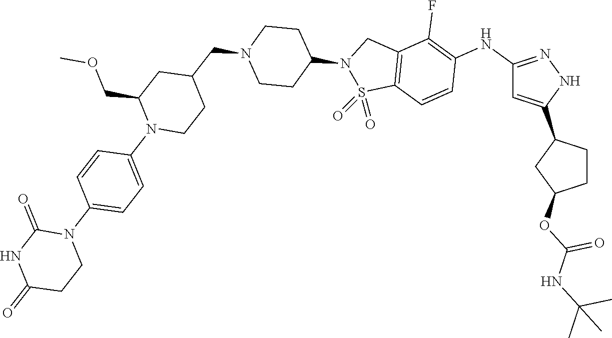

### Step 1: (R)-1-(4-(2-(methoxymethyl)piperazin-1-yl)phenyl)dihydropyrimidine-2,4(1H,3H)-dione

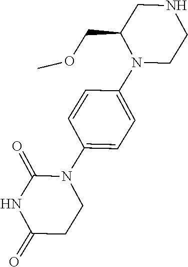 [SMILES: COC[C@H]1CNCCN1c1ccc(N2CCC(=O)NC2=O)cc1]

The titled compound was synthesized in the procedures similar to Example 80. LC-MS (ESI): m/z [M+H]⁺=319.2.

### Step 3: (1R,3S)-3-(3-((2-((1S,4R)-4-(((R)-4-(4-(2,4-dioxotetrahydropyrimidin-1(2H)-yl)phenyl)-3-(methoxymethyl)piperazin-1-yl)methyl)cyclohexyl)-4-fluoro-1,1-dioxido-2,3-dihydrobenzo[d]isothiazol-5-yl)amino)-1H-pyrazol-5-yl)cyclopentyl tert-butylcarbamate

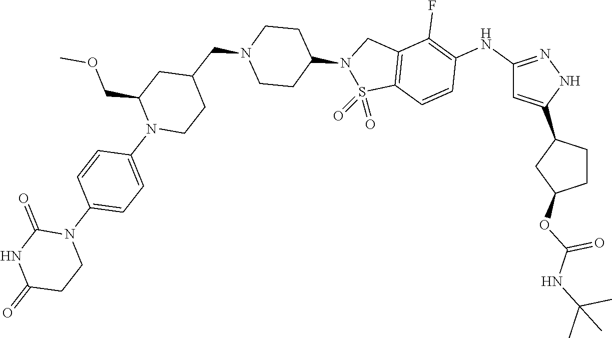

The titled compound was synthesized in the procedures similar to Example 79. ¹H NMR (500 MHz, DMSO) δ 11.98 (s, 1H), 10.25 (s, 1H), 8.76 (s, 1H), 8.30 (t, J=7.9 Hz, 1H), 7.50 (d, J=8.9 Hz, 1H), 7.14 (d, J=8.9 Hz, 2H), 6.88 (d, J=8.8 Hz, 2H), 6.76 (s, 1H), 5.83 (s, 1H), 5.06-4.96 (m, 1H), 4.52 (s, 2H), 3.96-3.90 (m, 1H), 3.72-3.64 (m, 3H), 3.48-3.44 (m, 1H), 3.21 (s, 3H), 3.19-3.14 (m, 1H), 3.11-2.92 (m, 4H), 2.86-2.82 (m, 1H), 2.71-2.62 (m, 3H), 2.46-2.44 (m, 1H), 2.23-2.07 (m, 4H), 2.04-1.82 (m, 6H), 1.78-1.47 (m, 6H), 1.21 (s, 9H), 1.10-0.99 (m, 2H). LC-MS (ESI): m/z [M+H]⁺=864.5;

### Example 86: (1R,3S)-3-(3-((2-((1S,4S)-4-(((S)-4-(4-(2,4-dioxotetrahydropyrimidin-1(2H)-yl)phenyl)-3-(methoxymethyl)piperazin-1-yl)methyl)cyclohexyl)-4-fluoro-1,1-dioxido-2,3-dihydrobenzo[d]isothiazol-5-yl)amino)-1H-pyrazol-5-yl)cyclopentyl tert-butylcarbamate

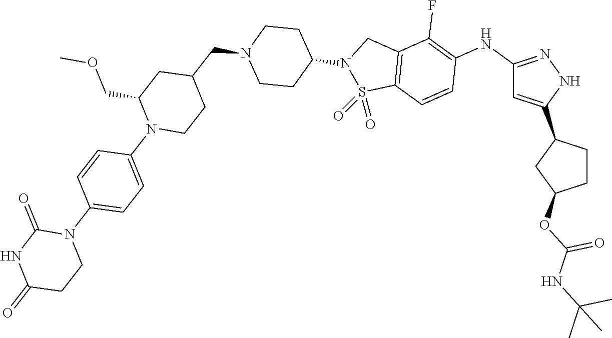

The titled compound was synthesized in the procedures similar to Example 85. ¹H NMR (500 MHz, DMSO) δ 11.98 (s, 1H), 10.25 (s, 1H), 8.76 (s, 1H), 8.30 (t, J=7.9 Hz, 1H), 7.50 (d, J=8.9 Hz, 1H), 7.14 (d, J=8.9 Hz, 2H), 6.88 (d, J=8.8 Hz, 2H), 6.76 (s, 1H), 5.83 (s, 1H), 5.06-4.96 (m, 1H), 4.52 (s, 2H), 3.96-3.90 (m, 1H), 3.72-3.64 (m, 3H), 3.48-3.44 (m, 1H), 3.21 (s, 3H), 3.19-3.14 (m, 1H), 3.11-2.92 (m, 4H), 2.86-2.82 (m, 1H), 2.71-2.62 (m, 3H), 2.46-2.44 (m, 1H), 2.23-2.07 (m, 4H), 2.04-1.82 (m, 6H), 1.78-1.47 (m, 6H), 1.21 (s, 9H), 1.10-0.99 (m, 2H). LC-MS (ESI): m/z [M+H]⁺=864.5;

### Example 87: (1R,3S)-3-(3-((2-(1-((1-(4-(2,4-dioxotetrahydropyrimidin-1(2H)-yl)-3-fluoro-2-methylphenyl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

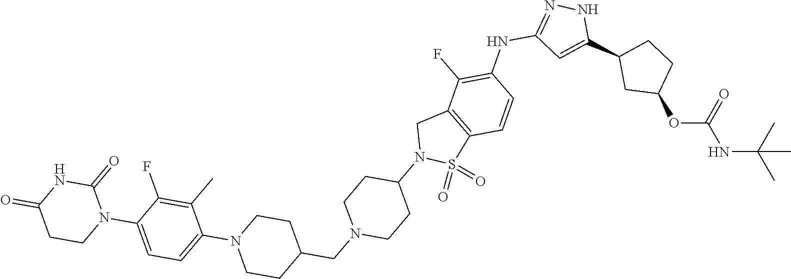 [SMILES: Cc1c(N2CCC(CN3CCC(N4Cc5c(ccc(Nc6cc([C@H]7CC[C@@H](OC(=O)NC(C)(C)C)C7)[nH]n6)c5F)S4(=O)=O)CC3)CC2)ccc(N2CCC(=O)NC2=O)c1F]

### Step 1: 1-(4-bromo-2-fluoro-3-methylphenyl)dihydropyrimidine-2,4(1H,3H)-dione

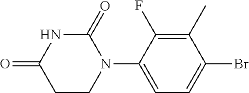 [SMILES: Cc1c(Br)ccc(N2CCC(=O)NC2=O)c1F]

To a mixture of t 4-bromo-2-fluoro-3-methylaniline (2 g, 9.8 mmol) and acrylic acid (706 mg, 9.8 mmol) in AcOH (30 mL) was stirred at 120° C. for 8 h. Then urea (1.18 g, 19.6 mmol) and H₂O (5 mL) was added and the mixture was stirred at 120° C. for another 16 h. The mixture was cooled to room temperature and concentrated under reduced pressure. DCM (100 mL) was added and washed with NaHCO₃ aq. (100 mL). The organic layer was dried over anhydrous Na₂SO₄. After filtration, the filtrate was concentrated under reduced pressure. The residue was purified by silica gel column chromatography, eluting with DCM/MeOH (1:20) to afford the product (1.7 g, 57% yield). LC-MS (ESI): m/z [M+H]⁺=301.3.

### Step 2: 1-(4-(4-(dimethoxymethyl)piperidin-1-yl)-2-fluoro-3-methylphenyl)dihydropyrimidine-2,4(1H,3H)-dione

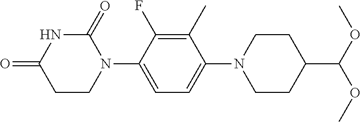 [SMILES: COC(OC)C1CCN(c2ccc(N3CCC(=O)NC3=O)c(F)c2C)CC1]

To a mixture of 1-(4-bromo-2-fluoro-3-methylphenyl)dihydropyrimidine-2,4(1H,3H)-dione (100 mg, 0.33 mmol) and 4-(dimethoxymethyl)piperidine (79 mg, 0.5 mmol) in andydrous DMA (5 mL) was added Pd2(dba)3 (60 mg, 0.07 mmol), RuPhos (61 mg, 0.13 mmol) and t-BuONa (127 mg, 1.33 mmol) was stirred under nitrogen atmosphere at 80° C. for 1 h. The mixture was diluted with EA (100 mL), washed with NH₄Cl aq. (2×50 mL) and brine (50 mL). The organic layer was dried over anhydrous Na₂SO₄, filtered and concentrated under reduced pressure. The residue was purified by silica gel column chromatography, eluting with DCM/MeOH (1:50) to afford the product (80 mg, 63% yield). LC-MS (ESI): m/z [M+H]⁺=380.5.

### Step 3: 1-(4-(2,4-dioxotetrahydropyrimidin-1(2H)-yl)-3-fluoro-2-methylphenyl)piperidine-4-carbaldehyde

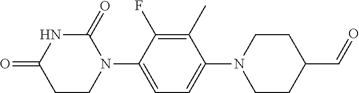 [SMILES: Cc1c(N2CCC(C=O)CC2)ccc(N2CCC(=O)NC2=O)c1F]

To a mixture of 1-(4-(4-(dimethoxymethyl)piperidin-1-yl)-2-fluoro-3-methylphenyl)dihydropyrimidine-2,4(1H,3H)-dione (80 mg, 0.21 mmol) in HCl aq (5 mL, 8 N) was stirred at room temperature for 3 h. The solution was cooled to 0° C., quenched with NaHCO₃ aq. (50 mL). The mixture was extracted with DCM (50 mL×3). The combined organic layers were washed with brine (50 mL) and dried over anhydrous Na₂SO₄. After filtration, the filtrate was concentrated under reduced pressure to afford the product (60 mg, 85% yield). LC-MS (ESI): m/z [M+H]⁺=334.5.

### Step 4: (1R,3S)-3-(3-((2-(1-((1-(4-(2,4-dioxotetrahydropyrimidin-1(2H)-yl)-3-fluoro-2-methylphenyl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

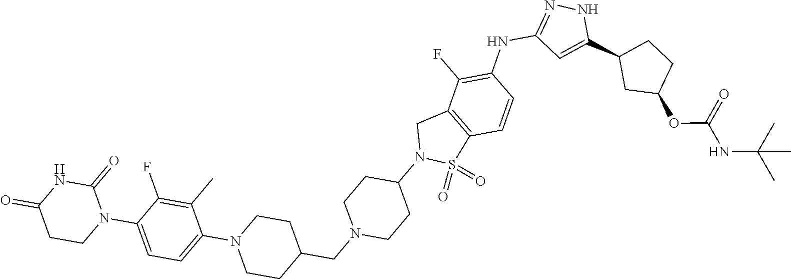 [SMILES: Cc1c(N2CCC(CN3CCC(N4Cc5c(ccc(Nc6cc([C@H]7CC[C@@H](OC(=O)NC(C)(C)C)C7)[nH]n6)c5F)S4(=O)=O)CC3)CC2)ccc(N2CCC(=O)NC2=O)c1F]

The titled compound was synthesized in the procedures similar to Example 70. ¹H NMR (500 MHz, DMSO) δ 10.42 (s, 1H), 9.06 (d, J=99.3 Hz, 1H), 8.86-8.82 (m, 1H), 8.33-8.28 (m, 1H), 7.58-7.54 (m, 1H), 7.20 (t, J=8.5 Hz, 1H), 6.89 (d, J=8.7 Hz, 1H), 6.75 (s, 1H), 5.84 (s, 1H), 4.98 (s, 1H), 4.57-4.56 (m, 3H), 3.8-3.77 (m, 1H), 3.67-3.63 (m, 3H), 3.16-3.04 (m, 6H), 2.71-2.64 (m, 5H), 2.36 (s, 1H), 2.19-2.16 (m, 6H), 2.03-2.00 (m, 2H), 1.93-1.85 (m, 3H), 1.73-1.70 (m, 2H), 1.58 (s, 1H), 1.42-1.42 (m, 2H), 1.24-1.21 (m 10H). LC-MS (ESI): m/z [M+H]⁺=852.76.

### Example 88a and 88b and 88c: (1R,3S)-3-(3-((2-(1-(((1S,4S,5S)-2-(4-(2,4-dioxotetrahydropyrimidin-1(2H)-yl)phenyl)-2-azabicyclo[2.2.1]heptan-5-yl)methyl)piperidin-4-yl)-4-fluoro-1,1-dioxido-2,3-dihydrobenzo[d]isothiazol-5-yl)amino)-1H-pyrazol-5-yl)cyclopentyl tertbutyl carbamate, (1R,3S)-3-(3-((2-(1-(((1S,4S,5R)-2-(4-(2,4-dioxotetrahydropyrimidin-1(2H)-yl)phenyl)-2-azabicyclo[2.2.1]heptan-5-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butyl carbamate and (1R,3S)-3-(3-((2-(1-(((1R,4R,5S)-2-(4-(2,4-dioxotetrahydropyrimidin-1(2H)-yl)phenyl)-2-azabicyclo[2.2.1]heptan-5-yl)methyl)piperidin-4-yl)-4-fluoro-1,1-dioxido-2,3-dihydrobenz-o[d]isothiazol-5-yl)amino)-1H-pyrazol-5-yl)cyclopentyl tert-butyl carbamate

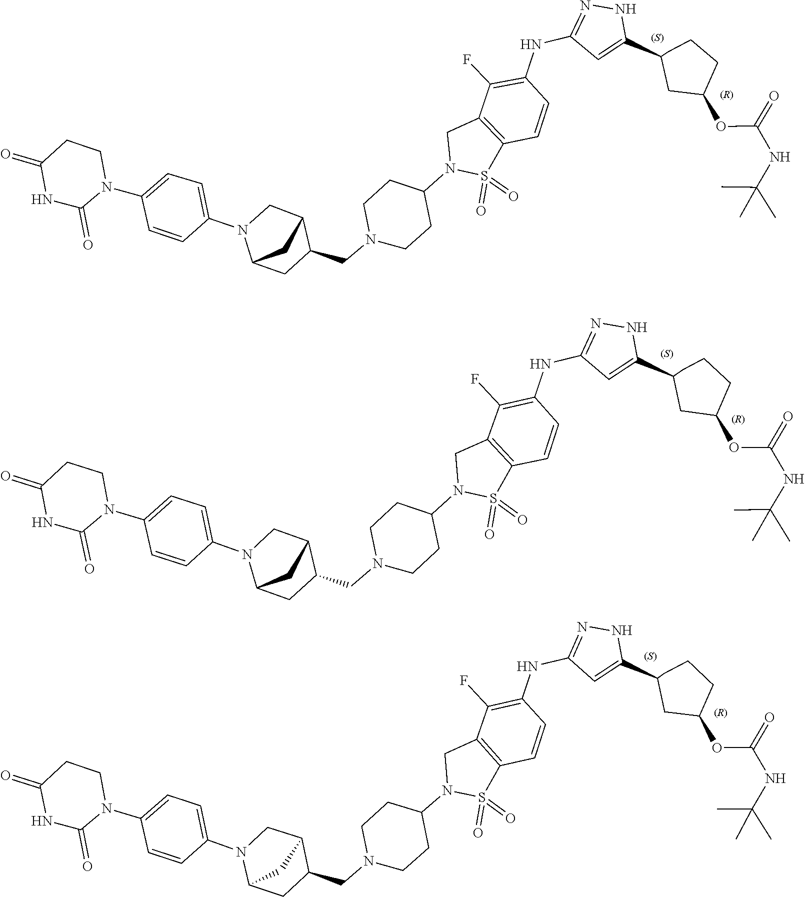

### Step 1: (2-azabicyclo[2.2.1]heptan-5-yl)methanol

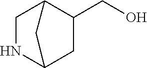

To a solution of tert-butyl 5-(hydroxymethyl)-2-azabicyclo[2.2.1]heptane-2-carboxylate (2 g, 8.81 mmol) in TFA (15 mL). The resulting mixture was stirred for 16 h. The mixture was concentrated under reduced pressure to afford the product (2 g, 100% yield).

### Step 2: (1R,3S)-3-(3-((2-(1-(((1S,4S,5S)-2-(4-(2,4-dioxotetrahydropyrimidin-1(2H)-yl)phenyl)-2-azabicyclo[2.2.1]heptan-5-yl)methyl)piperidin-4-yl)-4-fluoro-1,1-dioxido-2,3-dihydrobenzo[d]isothiazol-5-yl)amino)-1H-pyrazol-5-yl)cyclopentyl tertbutyl carbamate, (1R,3S)-3-(3-((2-(1-(((1S,4S,5R)-2-(4-(2,4-dioxotetrahydropyrimidin-1(2H)-yl)phenyl)-2-azabicyclo[2.2.1]heptan-5-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butyl carbamate and (1R,3S)-3-(3-((2-(1-(((1R,4R,5S)-2-(4-(2,4-dioxotetrahydropyrimidin-1(2H)-yl)phenyl)-2-azabicyclo[2.2.1]heptan-5-yl)methyl)piperidin-4-yl)-4-fluoro-1,1-dioxido-2,3-dihydrobenz-o[d]isothiazol-5-yl)amino)-1H-pyrazol-5-yl)cyclopentyl tert-butyl carbamate

The titled compound was synthesized in the procedures similar to Example 11 in a racemic form, which was further separated by chiral-HPLC to give:

Enantiomer 1 (Example 88a, 75.44% ee); Retention time: 6.613 min. ¹H NMR (500 MHz, DMSO) δ 11.98 (s, 1H), 10.20 (s, 1H), 8.77 (s, 1H), 8.30 (t, J=7.9 Hz, 1H), 7.50 (d, J=8.6 Hz, 1H), 7.05 (d, J=8.8 Hz, 2H), 6.76 (s, 1H), 6.49 (d, J=8.8 Hz, 2H), 5.83 (s, 1H), 4.96 (s, 1H), 4.50 (s, 2H), 4.11 (s, 1H), 3.73-3.61 (m, 2H), 3.46-3.38 (m, 2H), 3.11-2.84 (m, 3H), 2.76-2.62 (m, 4H), 2.46 (s, 2H), 2.30 (s, 1H), 2.12-1.99 (m, 3H), 1.92-1.70 (m, 8H), 1.61 (m, 3H), 1.31-1.17 (m, 11H). LC-MS (ESI): m/z [M+H]⁺=832.51.

Enantiomer 2 (Example 88b, 100% ee); Retention time: 8.49 min. ¹H NMR (500 MHz, DMSO) δ 11.98 (s, 1H), 10.20 (s, 1H), 8.77 (s, 1H), 8.30 (t, J=7.9 Hz, 1H), 7.50 (d, J=8.6 Hz, 1H), 7.05 (d, J=8.8 Hz, 2H), 6.76 (s, 1H), 6.49 (d, J=8.8 Hz, 2H), 5.83 (s, 1H), 4.96 (s, 1H), 4.50 (s, 2H), 4.11 (s, 1H), 3.73-3.61 (m, 2H), 3.46-3.38 (m, 2H), 3.11-2.84 (m, 3H), 2.76-2.62 (m, 4H), 2.46 (s, 2H), 2.30 (s, 1H), 2.12-1.99 (m, 3H), 1.92-1.70 (m, 8H), 1.61 (m, 3H), 1.31-1.17 (m, 11H). LC-MS (ESI): m/z [M+H]⁺=832.51.

Enantiomer 3 (Example 88c, 100% ee); Retention time: 16.821 min. ¹H NMR (500 MHz, DMSO) δ 11.98 (s, 1H), 10.20 (s, 1H), 8.76 (s, 1H), 8.30 (t, J=7.8 Hz, 1H), 7.50 (d, J=8.6 Hz, 1H), 7.06 (d, J=8.8 Hz, 2H), 6.76 (s, 1H), 6.49 (d, J=8.9 Hz, 2H), 5.83 (s, 1H), 4.98 (s, 1H), 4.50 (s, 2H), 4.09 (s, 1H), 3.66 (t, J=6.8 Hz, 2H), 3.40 (s, 1H), 3.30-2.26 (m, 1H), 3.10-2.93 (m, 3H), 2.82 (s, 1H), 2.70-2.63 (m, 3H), 2.56 (s, 2H), 2.29 (s, 1H), 2.15 (s, 2H), 2.08-1.99 (m, 2H), 1.92-1.78 (m, 7H), 1.75-1.68 (m, 2H), 1.66-1.55 (m, 2H), 1.25-1.07 (m, 10H). LC-MS (ESI): m/z [M+H]⁺=832.50.

Chiral analytical method: Column: YMC Amylose-SA 4.6*250 mm 5 m Mobile phase: A for MtBE (0.1%2M NH3 MeOH) (%) and B for MeOH:DCM=50:50 (v/v); Gradient: Mobile Phase A: Mobile Phase B=40:60 (v/v); HPLC Equipment: Agilent 1260 Infinity II (ARD_LC-808); Column temperature: 25° C.

Chiral Prep-HPLC Condition: YMC Amylose-SA 4.6*250 mm 5 m Mobile phase: A for MtBE (0.1%2M NH3 MeOH)(%) and B for MeOH:DCM=50:50; Gradient: Mobile Phase A: Mobile Phase B=40:60 (v/v); Flow Rate: 1 mL/min, Wavelength: UV 250 nm and 280 nm, Prep-HPLC Equipment: Prep-HPLC Gilson (ARD_LC-808); Column temperature: 25° C.

### Example 89: (1R,3S)-3-(3-((2-(1-((1-(4-(2,4-dioxotetrahydropyrimidin-1(2H)-yl)-2-fluoro-3-methylphenyl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

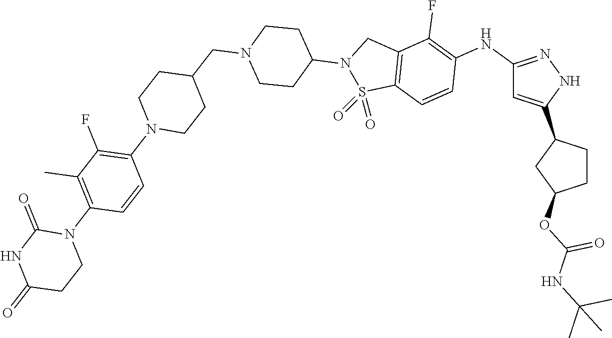 [SMILES: Cc1c(N2CCC(=O)NC2=O)ccc(N2CCC(CN3CCC(N4Cc5c(ccc(Nc6cc([C@H]7CC[C@@H](OC(=O)NC(C)(C)C)C7)[nH]n6)c5F)S4(=O)=O)CC3)CC2)c1F]

### Step 1: 3-((4-bromo-3-fluoro-2-methylphenyl)amino)propanoic acid

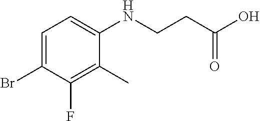

4-bromo-3-fluoro-2-methylaniline (2.0 g, 9.8 mmol) and acrylic acid (1.41 g, 19.6 mmol) were dissolved in AcOH (20 mL) and H₂O (4 mL), the reaction mixture was stirred for 16 hours at 110° C. The resulting mixture was cooled to room temperature, concentrated under reduce pressure, the residue was purified by column chromatography on silica gel PE/EA (1:1) to afford the product (2.65 g, 98%). LC-MS (ESI): m/z [M+H]⁺=276.3

### Step 2: 1-(4-bromo-3-fluoro-2-methylphenyl)dihydropyrimidine-2,4(1H,3H)-dione

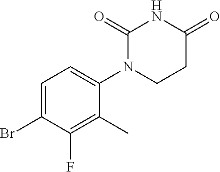 [SMILES: Cc1c(N2CCC(=O)NC2=O)ccc(Br)c1F]

3-((4-bromo-3-fluoro-2-methylphenyl)amino)propanoic acid (2.65 g, 9.6 mmol) was dissolved in AcOH (30 mL), urea (1.15 g, 19.2 mmol) was added, the reaction mixture was stirred for 16 hours at 110° C. The resulting mixture was cooled to room temperature, concentrated under reduce pressure, the residue was added saturated Na₂CO₃ aqueous, extracted with EtOAc for 3 times, the combined organic phase was washed with brine, dried over Na₂SO₄, filtered, the filtrated was concentrated under reduce pressure, the residue was purified by silica gel column chromatography, eluting with DCM/MeOH (20:1) to afford the product (1.17 g, 41% yield). LC-MS (ESI): m/z [M+H]⁺=301.2

### Step 3: 1-(4-(4-(dimethoxymethyl)piperidin-1-yl)-3-fluoro-2-methylphenyl) dihydropyrimidine-2,4(1H,3H)-dione

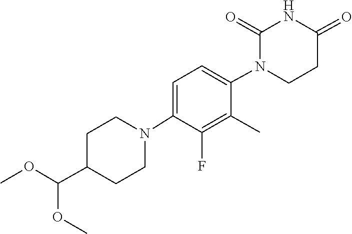 [SMILES: COC(OC)C1CCN(c2ccc(N3CCC(=O)NC3=O)c(C)c2F)CC1]

To a solution of 1-(4-bromo-3-fluoro-2-methylphenyl)dihydropyrimidine-2,4(1H,3H)-dione (170 mg, 0.57 mmol) and 4-(dimethoxymethyl)piperidine (180 mg, 1.13 mmol) in dioxane (50 mL) was added Pd-PEPPSI-IPentCl 2-methylpyridine (24 mg, 0.03 mmol) and Cs₂CO₃ (555 mg, 1.7 mmol), the reaction mixture was stirred for 16 hours at 100° C. under nitrogen. The resulting mixture was cooled to room temperature, filtered, the filtrate was concentrated under reduce pressure. The residue was purified by silica gel column chromatography, eluting with DCM/MeOH (20:1) to afford the product (61 mg, 28% yield). LC-MS (ESI): m/z [M+H]⁺=380.2.

### Step 4: 1-(4-(2,4-dioxotetrahydropyrimidin-1(2H)-yl)-2-fluoro-3-methylphenyl) piperidine-4-carbaldehyde

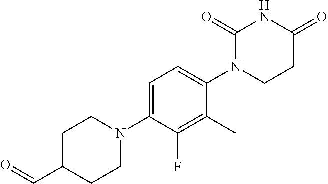 [SMILES: Cc1c(N2CCC(=O)NC2=O)ccc(N2CCC(C=O)CC2)c1F]

1-(4-(4-(dimethoxymethyl)piperidin-1-yl)-3-fluoro-2-methylphenyl) dihydropyrimidine-2,4(1H,3H)-dione (61 mg, 0.16 mmol) was dissolved in formic acid (1 mL), stirred for 2 hours at room temperature. The resulting mixture was concentrated under reduce pressure, dried over in vacuum, to afford the product (56 mg, 100%). LC-MS (ESI): m/z [M+H]⁺=334.4.

### Step 5: (1R,3S)-3-(3-((2-(1-((1-(4-(2,4-dioxotetrahydropyrimidin-1(2H)-yl)-2-fluoro-3-methylphenyl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

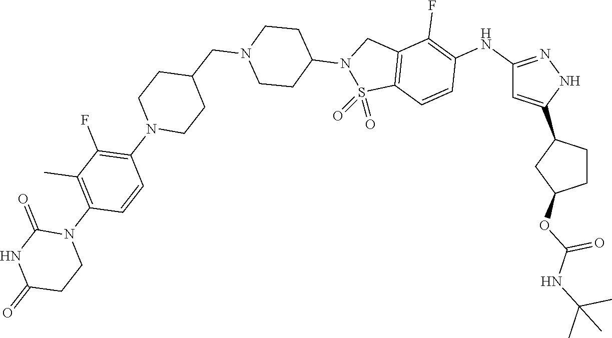 [SMILES: Cc1c(N2CCC(=O)NC2=O)ccc(N2CCC(CN3CCC(N4Cc5c(ccc(Nc6cc([C@H]7CC[C@@H](OC(=O)NC(C)(C)C)C7)[nH]n6)c5F)S4(=O)=O)CC3)CC2)c1F]

The titled compound was synthesized in the procedures similar to Example 87. LC-MS (ESI): m/z [M+H]⁺=852.59; ¹H NMR (500 MHz, DMSO) δ=11.98 (s, 1H), 10.34 (s, 1H), 8.77 (s, 1H), 8.29 (s, 1H), 7.51 (d, J=8.7, 1H), 7.02 (d, J=8.6, 1H), 6.90 (t, J=9.0, 1H), 6.76 (s, 1H), 5.83 (s, 1H), 4.97 (s, 1H), 4.52 (s, 2H), 3.75-3.71 (m, 1H), 3.49 (m, 2H), 3.09-3.02 (m, 1H), 2.91 (m, 2H), 2.80-2.74 (m, 2H), 2.66 (m, 4H), 2.47-2.44 (m, 1H), 2.21 (d, J=6.7, 2H), 2.05 (m, 6H), 1.88 (m, 5H), 1.81 (m, 2H), 1.70 (m, 2H), 1.61 (m, 2H), 1.27 (m, 2H), 1.21 (s, 9H).

### Example 90: (1R,3S)-3-(3-((2-(1-((1-(3-cyano-4-(2,4-dioxotetrahydropyrimidin-1(2H)-yl)phenyl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

### Step 1: 2-(2,4-dioxotetrahydropyrimidin-1(2H)-yl)-5-(4-formylpiperidin-1-yl)benzonitrile

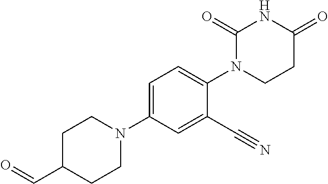 [SMILES: N#Cc1cc(N2CCC(C=O)CC2)ccc1N1CCC(=O)NC1=O]

1-(4-(4-(dimethoxymethyl)piperidin-1-yl)-2-(trifluoromethyl)phenyl)dihydropyrimidine-2,4(1H,3H)-dione (50.0 mg, 0.13 mmol, Vendor Acinopeptide Co; Cat #A2758531632SN) was dissolved in formic acid (3 mL), then the mixture was stirred for overnight at rt. The reaction was detected by LCMS, after completion the solution was concentrated under reduced pressure and used for the next step directly. (42.0 mg, quantitative yield). LC-MS (ESI): m/z [M+H]⁺=327.4.

### Step 2: (1R,3S)-3-(3-((2-(1-((1-(3-cyano-4-(2,4-dioxotetrahydropyrimidin-1(2H)-yl)phenyl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

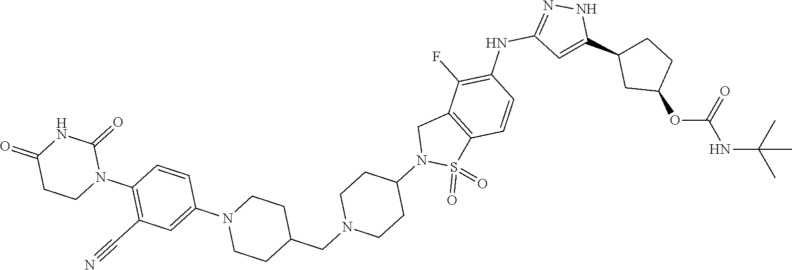 [SMILES: CC(C)(C)NC(=O)O[C@@H]1CC[C@H](c2cc(Nc3ccc4c(c3F)CN(C3CCN(CC5CCN(c6ccc(N7CCC(=O)NC7=O)c(C#N)c6)CC5)CC3)S4(=O)=O)n[nH]2)C1]

To a solution of 2-(2,4-dioxotetrahydropyrimidin-1(2H)-yl)-5-(4-formylpiperidin-1-yl)benzonitrile (42.0 mg, 0.13 mmol) in DMA (3 mL), (1R,3S)-3-(3-((4-fluoro-1,1-dioxido-2-(piperidin-4-yl)-2,3-dihydrobenzo[d]isothiazol-5-yl)amino)-1H-pyrazol-5-yl)cyclopentyl tert-butylcarbamate (69.42 mg, 0.13 mmol) were added at rt. The resulting mixture was stirred for 10 mins. Then sodium triacetoxyborohydride (83.0 mg, 0.39 mmol) was added into the mixture and stirred for 2 h. The reaction was detected by LCMS, after the residue was purified by silica gel chromatography, eluting with DCM/MeOH (10:1; R_{f}=0.31) to afford the crude product, then the crude product was purified by was purified by Prep-HPLC (Column: Xselect C18, 19*150 mm, 5 μm; Mobile Phase A: H₂O (0.1% FA), Mobile Phase B: ACN; Flow Rate: 17 mL/min; Room Temperature; Detector: UV 214 & 254 nm; Gradient: 23% to 45% B in 11 min, 45% to 90% B in 0.2 min, 90% B hold 2 min, 90% to 23% B in 0.3 min, 23% B hold 1.5 min. to give the product (17.07 mg, 16% yield). ¹H NMR (500 MHz, DMSO-d₆) δ 10.52 (s, 1H), 8.77 (s, 1H), 8.28 (t, J=8.0 Hz, 1H), 8.16 (s, 1H), 7.51 (d, J=8.6 Hz, 1H), 7.34 (dd, J=5.5, 3.1 Hz, 2H), 7.28 (dd, J=9.2, 2.1 Hz, 1H), 6.76 (s, 1H), 5.83 (s, 1H), 4.98 (s, 1H), 4.52 (s, 2H), 3.80 (d, J=10 Hz, 2H), 3.71 (m, 3H), 3.41 (m, 2H), 3.04 (m, 1H), 2.88-2.94 (m, 3H), 2.74 (m, 4H), 2.19 (d, J=10.7 Hz, 2H), 2.02 (m, 3H), 1.90 (m, 4H), 1.78-1.72 (m, 5H), 1.59 (m, 1H), 1.21 (s, 9H), 1.18-1.16 (d, J=10.0 Hz, 1H). LC-MS (ESI): m/z [M+H]⁺=845.43.

### Example 91: (1R,3S)-3-(3-((2-(1-((1-(7-(2,4-dioxotetrahydropyrimidin-1(2H)-yl)benzo[d][1,3]dioxol-4-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

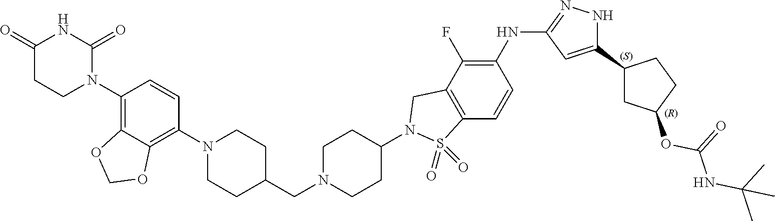

### Step 1: 7-bromobenzo[d][1,3]dioxol-4-amine

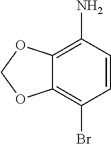 [SMILES: Nc1ccc(Br)c2c1OCO2]

To a solution of benzo[d][1,3]dioxol-4-amine (8 g, 58.4 mmol) in DMF (100 mL) was added NBS (10.9 g, 61.3 mmol). The resulting solution was stirred for 1 h at −30° C. under nitrogen atmosphere. The resulting solution was extracted with 3×100 mL of EtOAc. The combined organic phases were concentrated under reduced pressure. The residue was purified by combi-flash (EtOAc/PE=44%) to give the product (11.3 g, 89.6%) as red solid.

### Step 2: 3-((7-bromobenzo[d][1,3]dioxol-4-yl) amino) propanoic acid

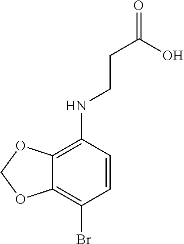 [SMILES: O=C(O)CCNc1ccc(Br)c2c1OCO2]

To a solution of 7-bromobenzo[d][1,3]dioxol-4-amine (11.2 g, 51.6 mmol) in H₂O (150 mL) and HOAc (30 mL) was added acrylic acid (4.1 g, 51.6 mmol) stirred for 12 h at 100° C. under nitrogen atmosphere. The resulting solution was extracted with 3×100 mL of EtOAc. The combined organic phases were concentrated under reduced pressure. The residue was purified by combi-flash (EtOAc/PE=50%) to give the product (7.2 g, 48.3%) as red solid. MS (ESI) m/e [M−1]⁻=286/288.

### Step 3: 1-(7-bromobenzo[d][1,3]dioxol-4-yl)dihydropyrimidine-2,4(1H,3H)-dione

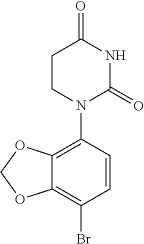 [SMILES: O=C1CCN(c2ccc(Br)c3c2OCO3)C(=O)N1]

To a solution of 3-((7-bromobenzo[d][1,3]dioxol-4-yl) amino) propanoic acid (7 g, 24.3 mmol) in HOAc (80 mL) was added NaOCN (3.9 g, 60.8 mmol). The resulting solution was stirred for 20 h at 60° C. Then was added 2M HCl (80 mL) stirred for 6 h at 60° C. under nitrogen atmosphere. The resulting solution was extracted with 3×100 mL of EtOAc. The combined organic phases were concentrated under reduced pressure. The residue was purified by combi-flash (EtOAc/PE=76%) to give the product (6.5 g, 85.5%) as red solid. MS (ESI) m/e [M+1]⁺=313/315.

### Step 4: 1-(7-(4-(dimethoxymethyl)piperidin-1-yl)benzo[d][1,3]dioxol-4-yl)-dihydropyrimidine-2,4(1H,3H)-dione

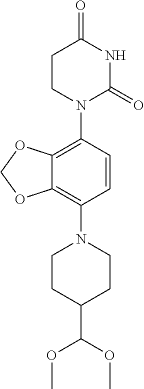 [SMILES: COC(OC)C1CCN(c2ccc(N3CCC(=O)NC3=O)c3c2OCO3)CC1]

To a solution of 1-(7-bromobenzo[d][1,3]dioxol-4-yl) dihydropyrimidine-2,4(1H,3H)-dione (4 g, 12.8 mmol) in dioxane (80 mL) were added 4-(dimethoxymethyl)piperidine (2.8 g, 17.9 mmol), Pd-PEPPSI-IPentCl 2-methylpyridine (o-picoline) (537.6 mg, 0.6 mmol) and Cs₂CO₃ (13 g, 38.4 mmol). The resulting solution was stirred for 12 h at 100° C. under nitrogen atmosphere. The resulting mixture was concentrated under vacuum. The residue was purified by combi-flash (EtOAc/PE=99%) to give the product (1.7 g, 38.6%) as light yellow solid. MS (ESI) m/e [M+1]⁺=392.

### Step 1: 1-(4-(6,6-dimethoxy-2-azaspiro[3.3]heptan-2-yl)phenyl)-3-((2-(trimethylsilyl)e-thoxy)methyl)dihydropyrimidine-2,4(1H,3H)-dione

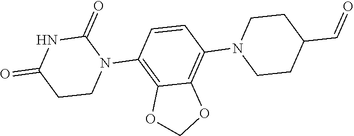 [SMILES: O=CC1CCN(c2ccc(N3CCC(=O)NC3=O)c3c2OCO3)CC1]

To a flask was added 1-(4-(6-((benzyloxy)methyl)-2-azaspiro[3.3]heptan-2-yl)phenyl)-3-((2-(trimethylsilyl)ethoxy)methyl)dihydropyrimidine-2,4(1H,3H)-dione (500 mg, 1.28 mmol) and Formic acid (10 mL). The reaction mixture was stirred for 1 hours at room temperature. The resulting mixture was concentrated to afford the product (440 mg, 100% yield). LC-MS (ESI): m/z [M+H]⁺=346.2.

### Step 2: (1R,3S)-3-(3-((2-(1-((1-(7-(2,4-dioxotetrahydropyrimidin-1(2H)-yl)benzo[d][1,3]dioxol-4-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

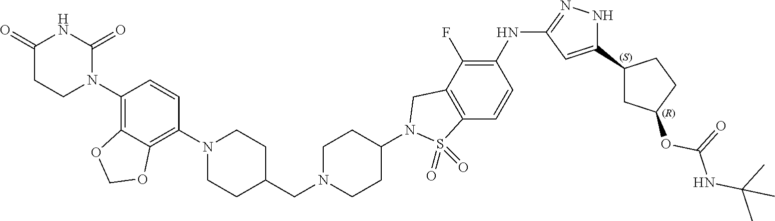

To a solution of (1R,3S)-3-(3-((4-fluoro-1,1-dioxido-2-(piperidin-4-yl)-2,3-dihydrobenz-o[d]isothiazol-5-yl)amino)-1H-pyrazol-5-yl)cyclopentyl tert-butylcarbamate (30 mg, 0.056 mmol) and 2-(4-(2,4-dioxotetrahydropyrimidin-1(2H)-yl)phenyl)-2-azaspiro[3.3]heptane-6-carbaldehyde (29 mg, 0.084 mmol) in DMA (1.5 mL) was stirred for 30 min, then STAB (24 mg, 0.112 mmol) was added. the reaction mixture was stirred for 16 hours at room tempe-rature. The resulting mixture was concentrated under reduce pressure, the residue was purified by column chromatography on silica gel, eluting with DCM/MeOH (10:1) to afford the product (20 mg, 46%). The residue 20 mg was purified by prep HPLC (Waters XSelect C18: RD-CO-094 column, eluting with a gradient of acetonitrile/water containing 0.1% FA, 20%-40%) to afford the product (10 mg, 22%). LC-MS (ESI): m/z [M+H]⁺=864.48. ¹H NMR (500 MHz, DMSO) δ 11.99 (s, 1H), 10.36 (s, 1H), 8.78 (s, 1H), 8.29 (s, 1H), 7.52 (d, J=8.7 Hz, 1H), 6.74 (d, J=8.9 Hz, 2H), 6.48 (d, J=8.9 Hz, 1H), 5.99 (s, 2H), 5.83 (s, 1H), 4.98 (s, 1H), 4.53 (s, 2H), 3.64 (t, J=6.7 Hz, 2H), 3.59 (d, J=11.6 Hz, 2H), 3.51 (s, 1H), 3.06 (dt, J=16.7, 8.2 Hz, 2H), 2.71-2.63 (m, 5H), 2.56-2.54 (m, 5H), 2.06-1.87 (m, 6H), 1.82-1.66 (m, 5H), 1.58 (s, 1H), 1.28-1.19 (m, 11H).

### Example 92: (1R,3S)-3-(3-((2-(1-((1-(4-(2,4-dioxotetrahydropyrimidin-1(2H)-yl)phenyl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl bicyclo[1.1.1]pentan-1-ylcarbamate

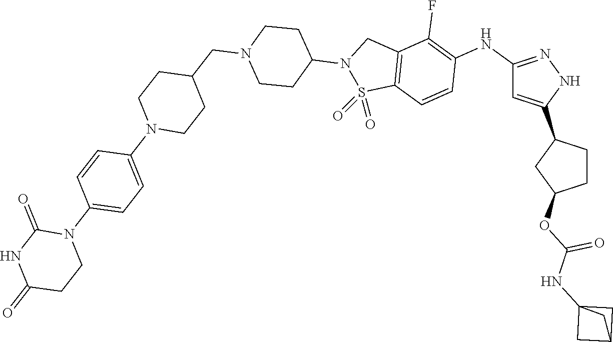 [SMILES: O=C1CCN(c2ccc(N3CCC(CN4CCC(N5Cc6c(ccc(Nc7cc([C@H]8CC[C@@H](OC(=O)NC9%10CC(C9)C%10)C8)[nH]n7)c6F)S5(=O)=O)CC4)CC3)cc2)C(=O)N1]

The titled compound was synthesized in the procedures similar to Example 70. ¹H NMR (500 MHz, DMSO) δ 11.99 (s, 1H), 10.24 (s, 1H), 8.76 (s, 1H), 8.29 (s, 1H), 7.75 (s, 1H), 7.51 (d, J=8.6 Hz, 1H), 7.13 (d, J=8.9 Hz, 2H), 6.92 (d, J=9.0 Hz, 2H), 5.83 (s, 1H), 5.06-4.96 (m, 1H), 4.52 (s, 2H), 3.72-3.64 (m, 4H), 3.46-3.40 (m, 1H), 3.14-3.03 (m, 1H), 2.91 (d, J=9.3 Hz, 2H), 2.72-2.63 (m, 6H), 2.39-2.33 (m, 1H), 2.21-2.15 (m, 2H), 2.11-1.99 (m, 3H), 1.90 (s, 11H), 1.82-1.53 (m, 6H), 1.28-1.12 (m, 2H). LC-MS (ESI): m/z [M+H]⁺=830.4;

### Example 93: (1R,3S)-3-(3-((2-(1-((1-(4-(2,4-dioxotetrahydropyrimidin-1(2H)-yl)phenyl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl (1-methylcyclopropyl)carbamate

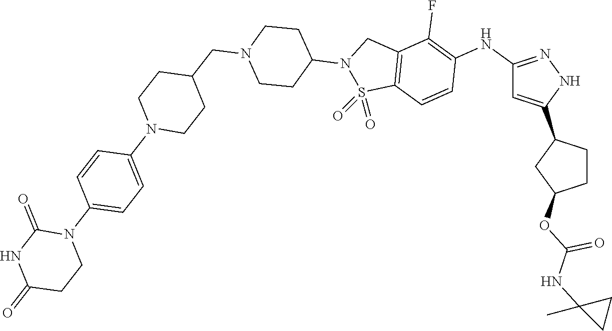 [SMILES: CC1(NC(=O)O[C@@H]2CC[C@H](c3cc(Nc4ccc5c(c4F)CN(C4CCN(CC6CCN(c7ccc(N8CCC(=O)NC8=O)cc7)CC6)CC4)S5(=O)=O)n[nH]3)C2)CC1]

The titled compound was synthesized in the procedures similar to Example 70. ¹H NMR (500 MHz, DMSO) δ 12.00 (s, 1H), 10.25 (s, 1H), 8.76 (s, 1H), 8.28 (s, 1H), 7.51 (d, J=8.7 Hz, 1H), 7.34 (s, 1H), 7.13 (d, J=8.9 Hz, 2H), 6.93 (d, J=8.9 Hz, 2H), 5.83 (s, 1H), 4.99 (s, 1H), 4.52 (s, 2H), 3.71-3.66 (m, 4H), 3.57-3.43 (m, 1H), 3.12-2.96 (m, 2H), 2.64-2.60 (m, 3H), 2.48-2.42 (m, 1H), 2.06-1.88 (m, 6H), 1.85-1.64 (m, 6H), 1.60-1.50 (m, 1H), 1.30-1.19 (m, 5H), 0.61-0.57 (m, 2H), 0.49-0.45 (m, 2H). LC-MS (ESI): m/z [M+H]⁺=818.4;

### Example 94: (1R,3S)-3-(3-((2-(1-((1-(1-(2,6-dioxopiperidin-3-yl)-3-methyl-2-oxo-2,3-dihydro-1H-benzo[d]imidazol-5-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

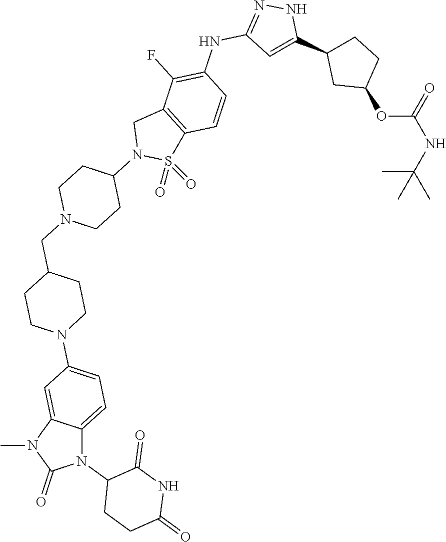 [SMILES: Cn1c(=O)n(C2CCC(=O)NC2=O)c2ccc(N3CCC(CN4CCC(N5Cc6c(ccc(Nc7cc([C@H]8CC[C@@H](OC(=O)NC(C)(C)C)C8)[nH]n7)c6F)S5(=O)=O)CC4)CC3)cc21]

The titled compound was synthesized in the procedures similar to Example 36. ¹H NMR (500 MHz, DMSO-d₆) δ 11.98 (s, 1H), 11.06 (s, 1H), 8.77 (s, 1H), 8.29 (s, 1H), 7.51 (d, J=10 Hz, 1H), 6.92 (d, J=10 Hz, 1H), 6.84-6.81 (m, 1H), 6.79-6.73 (m, 1H), 6.66-6.61 (m, 1H), 5.83 (s, 1H), 5.32-5.25 (m, 1H), 5.02-4.94 (m, 1H), 4.52 (s, 2H), 3.62-3.55 (m, 2H), 3.48-3.43 (m, 1H), 3.30 (s, 3H), 3.10-3.01 (m, 1H), 2.95-2.84 (m, 3H), 2.71-2.55 (m, 4H), 2.47-2.43 (m, 1H), 2.22-2.16 (m, 2H), 2.08-1.95 (m, 4H), 1.94-1.84 (m, 5H), 1.83-1.76 (m, 2H), 1.75-1.67 (m, 2H), 1.66-1.54 (m, 2H), 1.32-1.17 (m, 11H). LC-MS (ESI): m/z [M+H]⁺=889.

### Example 95: (1R,3S)-3-(3-((2-(1-((1-(3-(2,6-dioxopiperidin-3-yl)-2-oxo-2,3-dihydrobenzo[d]oxazol-6-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

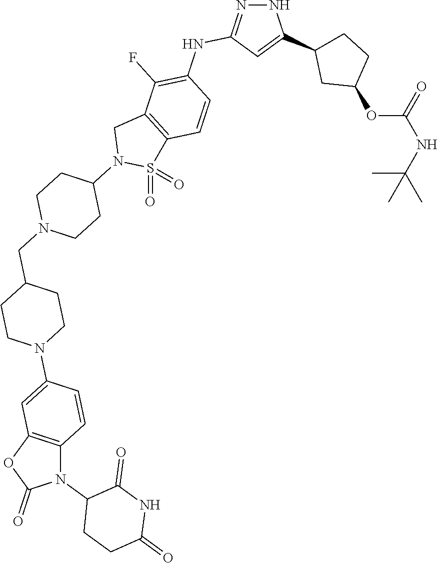 [SMILES: CC(C)(C)NC(=O)O[C@@H]1CC[C@H](c2cc(Nc3ccc4c(c3F)CN(C3CCN(CC5CCN(c6ccc7c(c6)oc(=O)n7C6CCC(=O)NC6=O)CC5)CC3)S4(=O)=O)n[nH]2)C1]

The titled compound was synthesized in the procedures similar to Example 36. ¹H NMR (500 MHz, DMSO-d₆) δ 11.99 (s, 1H), 11.18 (s, 1H), 8.77 (s, 1H), 8.28 (s, 1H), 7.51 (d, J=10 Hz, 1H), 7.09-7.03 (m, 2H), 6.82-6.72 (m, 2H), 5.83 (s, 1H), 5.34-5.25 (m, 1H), 5.02-4.96 (m, 1H), 4.52 (s, 2H), 3.63-3.55 (m, 2H), 3.47-3.40 (m, 1H), 3.12-3.01 (m, 1H), 2.94-2.82 (m, 3H), 2.71-2.58 (m, 4H), 2.47-2.43 (m, 1H), 2.22-2.09 (m, 3H), 2.07-1.95 (m, 3H), 1.95-1.83 (m, 5H), 1.81-1.68 (m, 4H), 1.66-1.54 (m, 2H), 1.27-1.15 (m, 11H). LC-MS (ESI): m/z [M+H]⁺=876.

### Example 96: (1R,3S)-3-(3-((2-(1-((1-(1-(2,6-dioxopiperidin-3-yl)-4-fluoro-3-methyl-2-oxo-2,3-dihydro-1H-benzo[d]imidazol-5-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

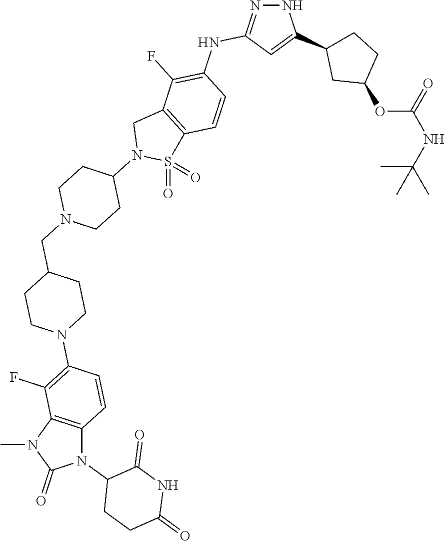 [SMILES: Cn1c(=O)n(C2CCC(=O)NC2=O)c2ccc(N3CCC(CN4CCC(N5Cc6c(ccc(Nc7cc([C@H]8CC[C@@H](OC(=O)NC(C)(C)C)C8)[nH]n7)c6F)S5(=O)=O)CC4)CC3)c(F)c21]

The titled compound was synthesized in the procedures similar to Example 36. ¹H NMR (500 MHz, DMSO-d₆) δ 11.99 (s, 1H), 11.07 (s, 1H), 8.77 (s, 1H), 8.29 (t, J=5 Hz, 1H), 7.51 (d, J=10 Hz, 1H), 6.94 (d, J=10 Hz, 1H), 6.68 (s, 1H), 6.55-6.49 (m, 1H), 5.83 (s, 1H), 5.34-5.25 (m, 1H), 5.02-4.96 (m, 1H), 4.52 (s, 2H), 3.63-3.55 (m, 2H), 3.47-3.40 (m, 1H), 3.30 (s, 3H), 3.12-3.01 (m, 1H), 2.94-2.82 (m, 3H), 2.71-2.58 (m, 4H), 2.47-2.43 (m, 1H), 2.22-2.09 (m, 3H), 2.07-1.95 (m, 3H), 1.95-1.83 (m, 5H), 1.81-1.68 (m, 4H), 1.67-1.53 (m, 2H), 1.28-1.14 (m, 11H). LC-MS (ESI): m/z [M+H]⁺=907.

### Example 97: (1S,3R)-3-(3-((2-((3R)-1-((1-(3-(2,6-dioxopiperidin-3-yl)-2-oxo-2,3-dihydrobenzo[d]oxazol-6-yl)piperidin-4-yl)methyl)pyrrolidin-3-yl)-4-fluoro-1,1-dioxido-2,3-dihydrobenzo[d]isothiazol-5-yl)amino)-1H-pyrazol-5-yl)cyclopentyl tert-butylcarbamate

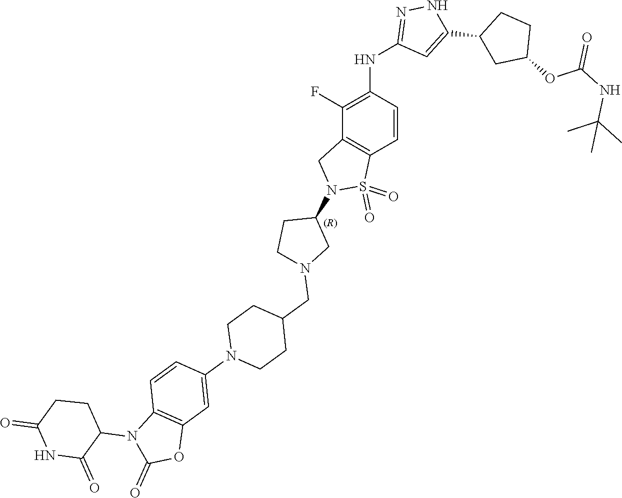

The titled compound was synthesized in the procedures similar to Example 36. ¹H NMR (500 MHz, DMSO-d₆) δ 11.99 (s, 1H), 11.17 (s, 1H), 8.78 (s, 1H), 8.36-8.23 (m, 1H), 7.51 (d, J=10 Hz, 1H), 7.09-6.99 (m, 2H), 6.82-6.68 (m, 2H), 5.84 (s, 1H), 5.36-5.23 (m, 1H), 5.08-4.93 (m, 1H), 4.64-4.48 (m, 2H), 4.21-4.10 (m, 1H), 3.67-3.53 (m, 2H), 3.12-2.99 (m, 1H), 2.94-2.73 (m, 3H), 2.70-2.54 (m, 5H), 2.47-2.43 (m, 1H), 2.40-2.22 (m, 3H), 2.21-2.08 (m, 2H), 2.08-1.96 (m, 2H), 1.95-1.86 (m, 1H), 1.86-1.77 (m, 2H), 1.76-1.64 (m, 2H), 1.64-1.52 (m, 2H), 1.33-1.11 (m, 11H). LC-MS (ESI): m/z [M+H]⁺=862.

### Example 98: (1R,3S)-3-(3-((2-((4S)-1-((1-(1-(2,6-dioxopiperidin-3-yl)-3-methyl-2-oxo-2,3-dihydro-1H-benzo[d]imidazol-5-yl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl tert-butylcarbamate

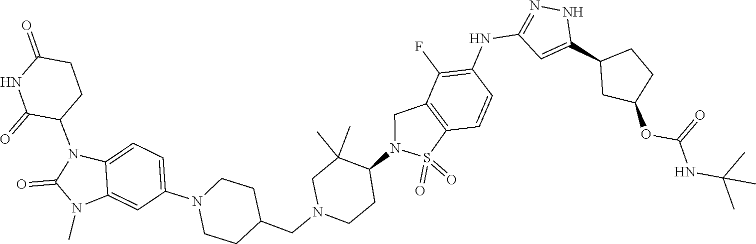 [SMILES: Cn1c(=O)n(C2CCC(=O)NC2=O)c2ccc(N3CCC(CN4CC[C@H](N5Cc6c(ccc(Nc7cc([C@H]8CC[C@@H](OC(=O)NC(C)(C)C)C8)[nH]n7)c6F)S5(=O)=O)C(C)(C)C4)CC3)cc21]

The titled compound was synthesized in the procedures similar to Example 36. LC-MS (ESI): m/z [M+H]⁺=917.62; ¹H NMR (500 MHz, DMSO) δ=11.98 (s, 1H), 11.06 (s, 1H), 8.77 (s, 1H), 8.31 (s, 1H), 7.54 (d, J=8.6, 1H), 6.92 (d, J=8.5, 1H), 6.83 (s, 1H), 6.76 (s, 1H), 6.64 (d, J=8.3, 1H), 5.84 (s, 1H), 5.28 (dd, J=12.7, 5.3, 1H), 4.96 (d, J=16.3, 1H), 4.55 (s, 2H), 3.59 (m, 3H), 3.22 (m, 1H), 3.08-3.04 (m, 1H), 2.92 (m, 2H), 2.67 (m, 5H), 2.59 (m, 2H), 2.44-2.40 (m, 1H), 2.22-2.15 (m, 2H), 2.14-2.09 (m, 1H), 1.98 (m, 3H), 1.90 (m, 1H), 1.80 (m, 3H), 1.72 (m, 2H), 1.61 (m, 2H), 1.51 (m, 1H), 1.21 (m, 11H), 1.13 (s, 3H), 0.94 (s, 3H).

### Example 99: (1R,3S)-3-(3-((2-((R)-1-((1-(1-((S)-2,6-dioxopiperidin-3-yl)-7-fluoro-3-methyl-2-oxo-2,3-dihydro-1H-benzo[d]imidazol-5-yl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl tert-butylcarbamate

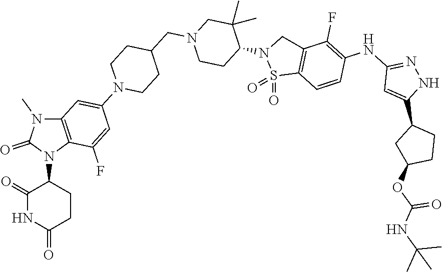 [SMILES: Cn1c(=O)n([C@H]2CCC(=O)NC2=O)c2c(F)cc(N3CCC(CN4CC[C@@H](N5Cc6c(ccc(Nc7cc([C@H]8CC[C@@H](OC(=O)NC(C)(C)C)C8)[nH]n7)c6F)S5(=O)=O)C(C)(C)C4)CC3)cc21]

The titled compound was synthesized in the procedures similar to Example 36 in a racemic form, which was further separated by Chiral Prep-HPLC to give Example 99 (100% ee); Retention time: 7.831 min. ¹H NMR (500 MHz, DMSO) δ 11.98 (s, 1H), 11.10 (s, 1H), 8.77 (s, 1H), 8.31 (t, J=7.8 Hz, 1H), 7.54 (d, J=8.7 Hz, 1H), 6.85 (d, J=8.4 Hz, 1H), 6.81-6.69 (m, 2H), 5.83 (s, 1H), 5.33 (dd, J=12.8, 5.4 Hz, 1H), 5.06-4.96 (m, 1H), 4.54 (s, 2H), 3.47 (s, 3H), 3.26-3.20 (m, 3H), 3.10-3.01 (m, 1H), 2.99-2.84 (m, 3H), 2.72-2.66 (m, 3H), 2.22-2.08 (m, 3H), 2.06-1.95 (m, 3H), 1.94-1.78 (m, 5H), 1.77-1.65 (m, 2H), 1.66-1.48 (m, 3H), 1.33-1.24 (m, 2H), 1.21 (s, 9H), 1.13 (s, 3H), 0.94 (s, 3H). LC-MS (ESI): m/z [M+H]⁺=935.4;

Chiral analytical method: Column: ICHIRALPAK SA 4.6*150 mm 5 μm; Mobile phase: A for MtBE (0.1%2M NH3 MeOH) (%) and B for MeOH:DCM=50:50(%); Gradient: Mobile Phase A: Mobile Phase B=60:40 (v/v); HPLC Equipment: HPLC-Agilent, Back pressure: 100 bar; Column temperature: 35° C.

Chiral Prep-HPLC Condition: CHIRALPAK SA 250*20.0 mml, 5 μm; Mobile phase: A for MtBE (0.1%2M NH3 MeOH) (%) and B for MeOH:DCM=50:50; Gradient: Mobile Phase A: Mobile Phase B=60:40 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 100: (1R,3S)-3-(3-((2-(1-((1-(3-(2,6-dioxopiperidin-3-yl)-2-oxo-2,3-dihydrobenzo[d]oxazol-6-yl)-4-methylpiperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

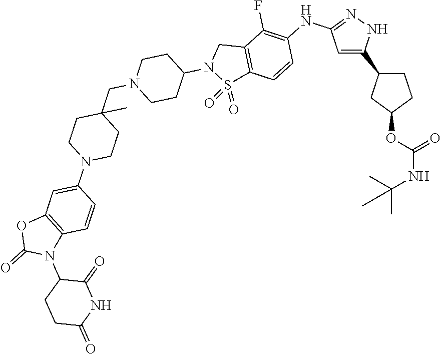 [SMILES: CC1(CN2CCC(N3Cc4c(ccc(Nc5cc([C@H]6CC[C@@H](OC(=O)NC(C)(C)C)C6)[nH]n5)c4F)S3(=O)=O)CC2)CCN(c2ccc3c(c2)oc(=O)n3C2CCC(=O)NC2=O)CC1]

### Step 1: 3-(2,6-bis(benzyloxy)pyridin-3-yl)-6-(4-(hydroxymethyl)-4-methylpiperidin-1-yl)benzo[d]oxazol-2(3H)-one

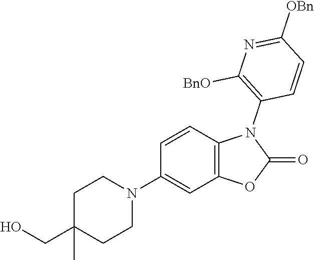 [SMILES: CC1(CO)CCN(c2ccc3c(c2)oc(=O)n3-c2ccc(OCc3ccccc3)nc2OCc2ccccc2)CC1]

To a mixture of 3-(2,6-bis(benzyloxy)pyridin-3-yl)-6-bromobenzo[d]oxazol-2(3H)-one (1 g, 1.99 mmol) and azetidin-3-ylmethanol (385 mg, 2.99 mmol) in andydrous 1,4-dioxane (10 mL) was added PEPPSI (167 mg, 0.20 mmol) and Cs₂CO₃ (1.95 g, 5.97 mmol). The reaction mixture was stirred under nitrogen atmosphere at 100° C. for 16 h. The mixture was filtered, and the filtrate was concentrated under reduced pressure. The residue was purified by silica gel column chromatography, eluting with PE/EA (1:9) to afford the product (500 mg, 46% yield). LC-MS (ESI): m/z [M+H]⁺=552.2.

### Step 2: 1-(4-(2,4-dioxo-3-((2-(trimethylsilyl)ethoxy)methyl)tetrahydropyrimidin-1(2H)-yl)phenyl)azetidine-3-carbaldehyde

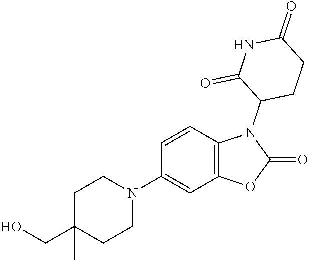 [SMILES: CC1(CO)CCN(c2ccc3c(c2)oc(=O)n3C2CCC(=O)NC2=O)CC1]

To a solution of 3-(2,6-bis(benzyloxy)pyridin-3-yl)-6-(4-(hydroxymethyl)-4-methylpiperidin-1-yl)benzo[d]oxazol-2(3H)-one (500 mg, 0.90 mmol) in THF (15 mL) was added Pd/C (10%, wet, 300 mg). The suspension was degassed and purged with hydrogen 3 times. The resulting mixture was stirred at 50° C. under a hydrogen balloon for 16 h. The mixture was filtered, and the filter cake was washed with EA (50 mL×3). The filtrate was concentrated under reduced pressure to afford the product (280 mg, 82% yield). LC-MS (ESI): m/z [M+H]⁺=374.3

### Step 3: 1-(3-(2,6-dioxopiperidin-3-yl)-2-oxo-2,3-dihydrobenzo[d]oxazol-6-yl)-4-methylpiperidine-4-carbaldehyde

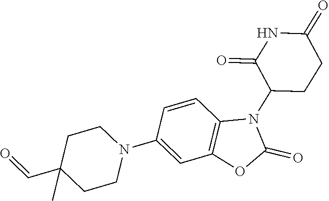 [SMILES: CC1(C=O)CCN(c2ccc3c(c2)oc(=O)n3C2CCC(=O)NC2=O)CC1]

To a stirred solution of 1-(4-(2,4-dioxo-3-((2-(trimethylsilyl)ethoxy)methyl)tetrahy-dropyrimidin-1(2H)-yl)phenyl)azetidine-3-carbaldehyde (300 mg, 0.80 mmol) in DMSO (5 mL) was added IBX (450 mg, 1.61 mmol). The resulting solution was stirred at room temperature for 16 h. quenched with NaHCO₃ (2 mL) The reaction mixture was poured into water (20 ml) and extracted with EA (50 mL×3). The combined organic layers were dried over anhydrous sodium sulfate, filtrated, and concentrated under reduced pressure to afford the crude product (150 mg, 50% yield). LC-MS (ESI): m/z [M+H]⁺=372.2.

### Step 4: (1R,3S)-3-(3-((2-(1-((1-(3-(2,6-dioxopiperidin-3-yl)-2-oxo-2,3-dihydrobenzo[d]oxazol-6-yl)-4-methylpiperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

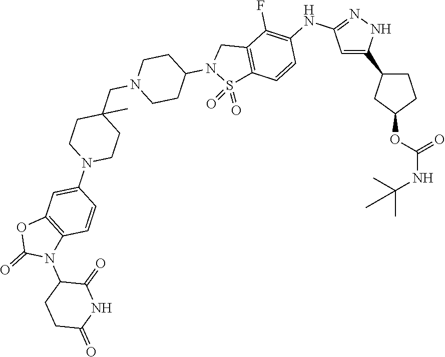 [SMILES: CC1(CN2CCC(N3Cc4c(ccc(Nc5cc([C@H]6CC[C@@H](OC(=O)NC(C)(C)C)C6)[nH]n5)c4F)S3(=O)=O)CC2)CCN(c2ccc3c(c2)oc(=O)n3C2CCC(=O)NC2=O)CC1]

To a solution of (1R,3S)-3-(3-((4-fluoro-1,1-dioxido-2-(piperidin-4-yl)-2,3-dihydrobenz-o[d]isothiazol-5-yl)amino)-1H-pyrazol-5-yl)cyclopentyl tert-butyl carbamate (80 mg, 0.150 mmol) and 1-(3-(2,6-dioxopiperidin-3-yl)-2-oxo-2,3-dihydrobenzo[d]oxazol-6-yl)-4-methylpiperidine-4-carbaldehyde (83 mg, 0.225 mmol) in DMA (1.5 mL) was stirred for 30 min, then STAB (63 mg, 0.300 mmol) was added. the reaction mixture was stirred for 16 hours at room tempe-rature. The resulting mixture was concentrated under reduce pressure, the residue was purified by column chromatography on silica gel, eluting with DCM/MeOH (10:1) to afford the product (20 mg, 15%). The residue 20 mg was purified by prep HPLC (Waters XSelect C18: RD-CO-094 column, eluting with a gradient of acetonitrile/water containing 0.1% FA, 20%-40%) to afford the product (9 mg, 8%).

¹H NMR (500 MHz, DMSO) δ 12.02 (s, 1H), 11.19 (s, 1H), 8.79 (s, 1H), 8.31-8.27 (m, 1H), 7.52 (d, J=8.6 Hz, 1H), 7.12-7.05 (m, 2H), 6.85-6.69 (m, 2H), 5.85 (s, 1H), 5.34-5.31 (m, 1H), 5.05-4.95 (m, 1H), 4.54 (s, 2H), 3.51-3.37 (m, 2H), 3.29-3.22 (m, 2H), 3.13-3.04 (m, 1H), 3.00-2.82 (m, 6H), 2.70-2.64 (m, 2H), 2.43-2.37 (m, 2H), 2.25-2.19 (s, 2H), 2.18-2.12 (m, 1H), 2.07-2.02 (m, 1H), 2.01-1.82 (m, 6H), 1.79-1.68 (m, 2H), 1.67-1.56 (m, 3H), 1.39-1.32 (m, 2H), 1.21 (s, 9H), 0.95-0.89 (m, 3H). LC-MS (ESI): m/z [M+H]⁺=890.48.

### Example 101: (1R,3S)-3-(3-((2-(1-((1-(3-(2,6-dioxopiperidin-3-yl)-2-oxo-2,3-dihydrobenzo[d]oxazol-6-yl)azetidin-3-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

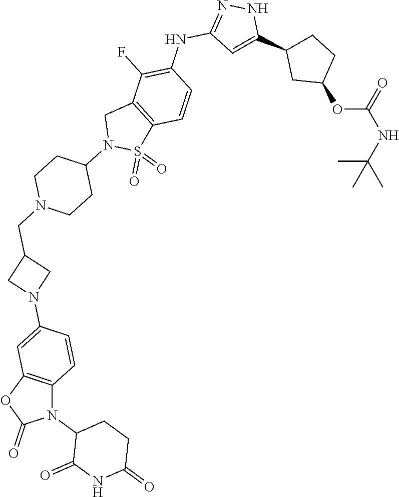 [SMILES: CC(C)(C)NC(=O)O[C@@H]1CC[C@H](c2cc(Nc3ccc4c(c3F)CN(C3CCN(CC5CN(c6ccc7c(c6)oc(=O)n7C6CCC(=O)NC6=O)C5)CC3)S4(=O)=O)n[nH]2)C1]

### Step 1: tert-butyl 3-((benzyloxy)methyl)azetidine-1-carboxylate

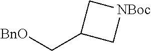 [SMILES: CC(C)(C)OC(=O)N1CC(COCc2ccccc2)C1]

To a solution of tert-butyl 3-(hydroxymethyl)azetidine-1-carboxylate (10 g, 5.35 mmol) in THF (200 mL) was added NaH (6.4 g, 16.05 mmol) at 0° C. After stirring of the reaction mixture for 0.5 h at 0° C. To the above mixture was added BnBr (13.7 g, 8.0 mmol) in portions at 0° C. The resulting mixture was stirred for additional 16 h at rt. The resulting mixture was extracted with DCM (3×200 mL). The combined organic layers were washed with brine (3×200 mL), dried over anhydrous Na₂SO₄. After filtration, the filtrate was concentrated under reduced pressure. The crude product (6 g, 40.5%) as colorless oil. LCMS (ESI) m/e [M+1]⁺=278

### Step 2: 3-((benzyloxy)methyl)azetidine

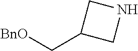 [SMILES: c1ccc(COCC2CNC2)cc1]

To a solution of tert-butyl 3-((benzyloxy)methyl)azetidine-1-carboxylate (5 g, 18.0 mmol) in DCM (60 mL) was added TFA (20 mL) at 0° C. After stirring of the reaction mixture for 2 h at rt. The reaction was concentrated under vacuum. The crude product (3.5 g, 90.0%) was used in the next step directly without further purification. LCMS (ESI) m/e [M+1]⁺=178

### Step 3: 6-(3-((benzyloxy)methyl)azetidin-1-yl)-3-(2,6-bis(benzyloxy)pyridin-3-yl)benzo[d]oxazol-2(3H)-one

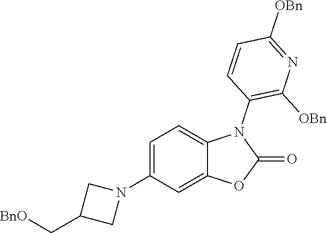 [SMILES: O=c1oc2cc(N3CC(COCc4ccccc4)C3)ccc2n1-c1ccc(OCc2ccccc2)nc1OCc1ccccc1]

To a solution of 3-(2,6-bis(benzyloxy)pyridin-3-yl)-6-bromobenzo[d]oxazol-2(3H)-one (4 g, 7.9 mmol) in dioxane (80 mL) were added 3-((benzyloxy)methyl)azetidine (2.1 g, 11.9 mmol), Pd-PEPPSI-IPentCl 2-methylpyridine (311.8 mg, 0.4 mmol) and Cs₂CO₃ (6.4 g, 19.7 mmol). The resulting solution was stirred for 3 h at 100° C. under nitrogen atmosphere. The resulting mixture was concentrated under vacuum. The residue was purified by combi-flash (EtOAc/PE=80%) to give the product (2 g, 41.9%) as yellow solid. LCMS (ESI) m/e [M+1]+=600.

### Step 4: 3-(6-(3-(hydroxymethyl)azetidin-1-yl)-2-oxobenzo[d]oxazol-3(2H)-yl)piperidine-2,6-dione

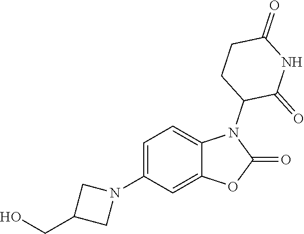 [SMILES: O=C1CCC(n2c(=O)oc3cc(N4CC(CO)C4)ccc32)C(=O)N1]

To a solution of 6-(3-((benzyloxy)methyl)azetidin-1-yl)-3-(2,6-bis(benzyloxy)pyridin-3-yl)benzo[d]oxazol-2(3H)-one (2 g, 3.3 mmol) in THF (60 mL) was added Pd/C (2 g, 10% wt). The resulting solution was stirred for 12 h at 50° C. under H₂ atmosphere. After filtration, the filtrate was concentrated under vacuum. The residue was the product (533.8 g, 48.3%) as yellow solid. LCMS (ESI) m/e [M+1]⁺=332.

### Step 5: 1-(3-(2,6-dioxopiperidin-3-yl)-2-oxo-2,3-dihydrobenzo[d]oxazol-6-yl)azetidine-3-carbaldehyde

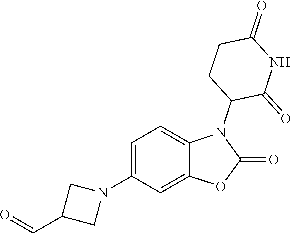 [SMILES: O=CC1CN(c2ccc3c(c2)oc(=O)n3C2CCC(=O)NC2=O)C1]

The titled compound was synthesized in the procedures similar to Example 74.

### Step 6: (1R,3S)-3-(3-((2-(1-((1-(3-(2,6-dioxopiperidin-3-yl)-2-oxo-2,3-dihydrobenzo[d]oxazol-6-yl)azetidin-3-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

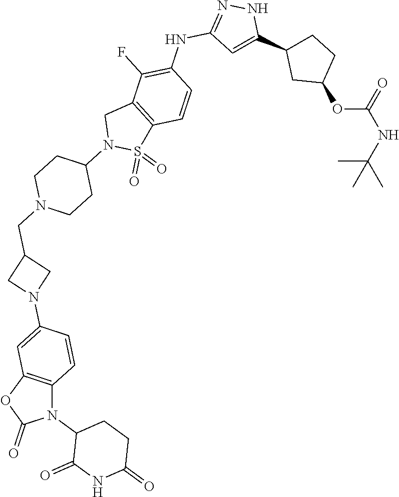 [SMILES: CC(C)(C)NC(=O)O[C@@H]1CC[C@H](c2cc(Nc3ccc4c(c3F)CN(C3CCN(CC5CN(c6ccc7c(c6)oc(=O)n7C6CCC(=O)NC6=O)C5)CC3)S4(=O)=O)n[nH]2)C1]

The titled compound was synthesized in the procedures similar to Example 36. ¹H NMR (500 MHz, DMSO-d₆) δ 11.99 (s, 1H), 11.16 (s, 1H), 8.77 (s, 1H), 8.28 (s, 1H), 7.51 (d, J=10 Hz, 1H), 7.04 (d, J=10 Hz, 1H), 6.82-6.72 (m, 1H), 6.56-6.50 (m, 1H), 6.26-6.20 (m, 1H), 5.83 (s, 1H), 5.31-5.22 (m, 1H), 5.02-4.93 (m, 1H), 4.51 (s, 2H), 3.90 (t, J=5 Hz, 2H), 3.41 (t, J=5 Hz, 2H), 3.12-3.01 (m, 1H), 2.94-2.82 (m, 4H), 2.69-2.55 (m, 4H), 2.47-2.43 (m, 1H), 2.16-1.97 (m, 4H), 1.95-1.80 (m, 5H), 1.76-1.55 (m, 2H), 1.63-1.54 (m, 1H), 1.21 (s, 9H). LC-MS (ESI): m/z [M+H]⁺=848.

### Example 102: (1R,3S)-3-(3-((2-((4S)-1-((1-(3-(2,6-dioxopiperidin-3-yl)-2-oxo-2,3-dihydrobenzo[d]oxazol-6-yl)azetidin-3-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl tert-butylcarbamate

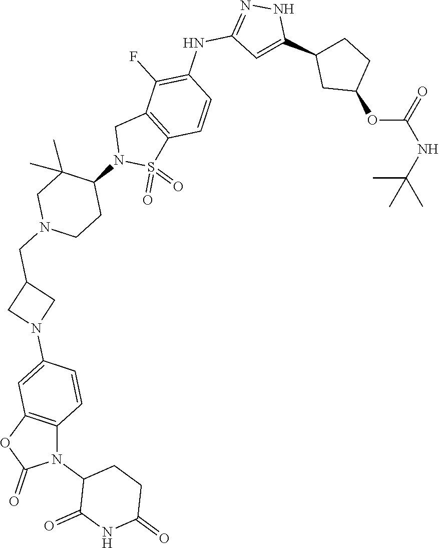 [SMILES: CC(C)(C)NC(=O)O[C@@H]1CC[C@H](c2cc(Nc3ccc4c(c3F)CN([C@H]3CCN(CC5CN(c6ccc7c(c6)oc(=O)n7C6CCC(=O)NC6=O)C5)CC3(C)C)S4(=O)=O)n[nH]2)C1]

The titled compound was synthesized in the procedures similar to Example 36. ¹H NMR (500 MHz, DMSO-d₆) δ 11.98 (s, 1H), 11.16 (s, 1H), 8.77 (s, 1H), 8.30 (s, 1H), 7.54 (d, J=10 Hz, 1H), 7.04 (d, J=5 Hz, 1H), 6.53 (s, 1H), 6.26-6.20 (m, 1H), 5.84 (s, 1H), 5.31-5.22 (m, 1H), 5.02-4.93 (m, 1H), 4.54 (s, 2H), 3.89 (t, J=5 Hz, 2H), 3.41 (t, J=5 Hz, 2H), 3.22-3.15 (m, 1H), 3.12-3.01 (m, 1H), 2.94-2.88 (m, 3H), 2.69-2.55 (m, 5H), 2.47-2.43 (m, 1H), 2.16-1.97 (m, 4H), 1.96-1.84 (m, 2H), 1.76-1.66 (m, 2H), 1.63-1.54 (m, 1H), 1.53. 1.46 (m, 1H), 1.21 (s, 9H), 1.11 (s, 3H), 0.93 (s, 3H). LC-MS (ESI): m/z [M+H]⁺=876.

### Example 103: (1R,3S)-3-(3-((2-((4S)-1-((1-(3-(2,6-dioxopiperidin-3-yl)-2-oxo-2,3-dihydrobenzo[d]oxazol-6-yl)azetidin-3-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl tert-butylcarbamate

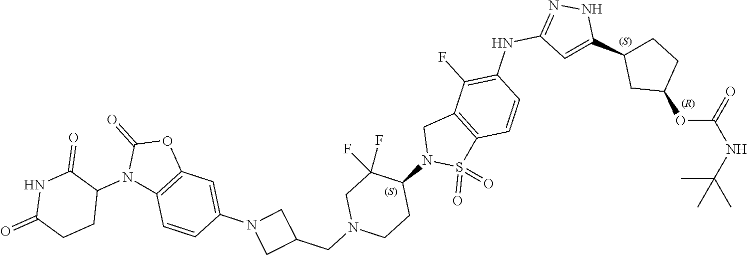

The titled compound was synthesized in the procedures similar to Example 101. ¹H NMR (500 MHz, DMSO) δ 12.00 (s, 1H), 11.17 (s, 1H), 8.83 (s, 1H), 8.33 (t, 1H), 7.58 (d, J=8.7 Hz, 1H), 7.05 (d, J=8.5 Hz, 1H), 6.77 (s, 1H), 6.54 (d, J=1.8 Hz, 1H), 6.24 (dd, J=8.5, 1.9 Hz, 1H), 5.84 (s, 1H), 5.27 (dd, J=12.9, 5.3 Hz, 1H), 4.98 (s, 1H), 4.75 (d, J=14.3 Hz, 1H), 4.53 (d, J=13.2 Hz, 1H), 4.05-3.94 (m, 1H), 3.90 (t, J=7.2 Hz, 2H), 3.44 (t, J=6.3 Hz, 2H), 3.17 (t, J=9.8 Hz, 1H), 3.10-3.02 (m, 1H), 2.98-2.84 (m, 3H), 2.71 (d, J=7.3 Hz, 2H), 2.67-2.62 (m, 3H), 2.44-2.33 (m, 2H), 2.24-2.08 (m, 2H), 2.06-1.98 (m, 1H), 1.96-1.86 (m, 1H), 1.84-1.65 (m, 3H), 1.59 (s, 1H), 1.21 (s, 9H). LC-MS (ESI): m/z [M+H]⁺=885.65

### Example 104: (1R,3S)-3-(3-((2-(1-((1-(1-((S)-2,6-dioxopiperidin-3-yl)-4,7-difluoro-3-methyl-2-oxo-2,3-dihydro-1H-benzo[d]imidazol-5-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

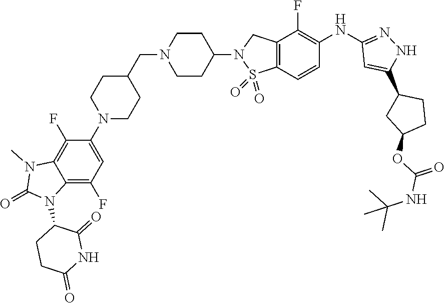 [SMILES: Cn1c(=O)n([C@H]2CCC(=O)NC2=O)c2c(F)cc(N3CCC(CN4CCC(N5Cc6c(ccc(Nc7cc([C@H]8CC[C@@H](OC(=O)NC(C)(C)C)C8)[nH]n7)c6F)S5(=O)=O)CC4)CC3)c(F)c21]

The titled compound was synthesized in the procedures similar to Example 55 in a racemic form, which was further separated by Chiral Prep-HPLC to give Example 104 (100% ee); Retention time: 7.39 min. ¹H NMR (500 MHz, DMSO) δ 11.98 (s, 1H), 11.11 (s, 1H), 8.77 (s, 1H), 8.30 (s, 1H), 7.51 (d, J=8.7 Hz, 1H), 6.76 (s, 1H), 6.66 (d, J=7.3 Hz, 1H), 5.83 (s, 1H), 5.45 (s, 1H), 4.98 (s, 1H), 4.52 (s, 2H), 3.50 (s, 3H), 3.46-3.39 (m, 2H), 3.25 (s, 2H), 3.09-2.94 (m, 2H), 2.91-2.89 (m, 2H), 2.66-2.61 (m, 3H), 2.46-2.44 (m, 4H), 2.20-2.19 (m, 3H), 2.09-2.01 (m, 2H), 1.89-1.85 (m, 3H), 1.81-1.79 (m, 2H), 1.73-1.68 (m, 2H), 1.61 (s, 2H), 1.33-1.26 (m, 2H), 1.21 (s, 9H). LC-MS (ESI): m/z [M+H]⁺=925.38.

Chiral analytical method: Column: I-Cellulose-5, 4.6 mm×250 mm, 5 um; Mobile phase: A for Hexane and B for EtOH (0.1% 2M NH₃ MeOH); Gradient: Mobile Phase A: Mobile Phase B=30:70 (v/v); HPLC Equipment: HPLC-Agilent, Back pressure: 100 bar; Column temperature: 35° C.

Chiral Prep-HPLC Condition: Column: I-Cellulose-5, 21.2 mm×250 mm, 5 um; Mobile phase: A for Hexane and B for EtOH (0.2% 2M NH₃ MeOH); Gradient: Mobile Phase A: Mobile Phase B=30:70 (v/v); Flow Rate: 20 mL/min, Wave Length: UV 200 nm and 270 nm, Prep-HPLC Equipment: Prep-HPLC-Gilson, Back pressure: 100 bar; Column temperature: 25° C.

### Example 105: (1R,3S)-3-(3-((2-((4R)-1′-(1-(2,6-dioxopiperidin-3-yl)-4-methoxy-3-methyl-2-oxo-2,3-dihydro-1H-benzo[d]imidazol-5-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl tert-butylcarbamate

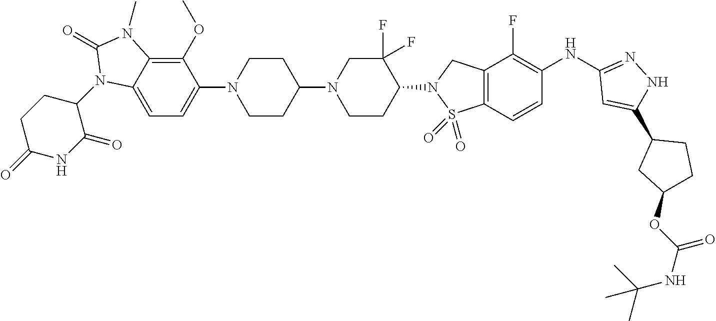 [SMILES: COc1c(N2CCC(N3CC[C@@H](N4Cc5c(ccc(Nc6cc([C@H]7CC[C@@H](OC(=O)NC(C)(C)C)C7)[nH]n6)c5F)S4(=O)=O)C(F)(F)C3)CC2)ccc2c1n(C)c(=O)n2C1CCC(=O)NC1=O]

### Step 1: 4-bromo-3-methoxy-2-nitroaniline

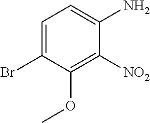

To a solution of 4-bromo-3-fluoro-2-nitroaniline (10 g, 42.7 mmol) in MeOH (500 mL) was added NaOMe (30%) (138 g, 769 mmol) at rt for overnight. The resulting mixture was concentrated under vacuum and extracted with DCM. The organic layers were washed with brine and concentrated under vacuum. The residue was purified by combi-flash (DCM/EA=100:0 to 87:13) to give the product (10 g, 95.2%) as yellow solid.

### Step 2: 5-bromo-4-methoxy-1,3-dihydro-2H-benzo[d]imidazol-2-one

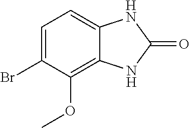

To a solution of 4-bromo-3-methoxy-2-nitroaniline (11 g, 44.7 mmol) in DMF (200 mL) were added B₂(OH)₄ (12 g, 134.14 mmol) and 4,4-Bipyridine (349 mg, 2.23 mmol) at 0° C. for 15 min under N₂. The CDI (14.48 g, 89.4 mmol) was added in it at rt for 1 h. The resulting solution was quenched with NH₄Cl solution and extracted with EA. The organic layers were washed brine and concentrated under vacuum. The filtrate was concentrated under vacuum. The residue was purified by combi-flash (DCM/MeOH=100:0 to 80:20) to give the product (5.2 g, 52.9%) as brown solid.

### Step 3:1-(2,6-bis(benzyloxy)pyridin-3-yl)-5-bromo-4-methoxy-1,3-dihydro-2H-benzo[d]imidazol-2-one

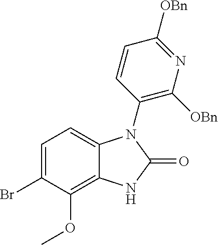 [SMILES: COc1c(Br)ccc2c1[nH]c(=O)n2-c1ccc(OCc2ccccc2)nc1OCc1ccccc1]

To a solution of 5-bromo-4-methoxy-1,3-dihydro-2H-benzo[d]imidazol-2-one (5.2 g, 21.48 mmol) in dioxane (100 mL) were added 2,6-bis(benzyloxy)-3-(4,4,5,5-tetramethyl-1,3,2-dioxaborolan-2-yl)pyridine (10.8 g, 33.23 mmol), Py (5.09 g, 64.44 mmol), Cu(OAc)₂ (4.27 g, 21.48 mmol) and 4AMS (2.5 g) at 80° C. for overnight under 02. The reaction was concentrated under vacuum. The residue was purified by combi-flash (PE/EA=100:0 to 67:37) to give the product (2 g, 10.5%) as light brown solid.

### Step 4: 1-(2,6-bis(benzyloxy)pyridin-3-yl)-5-bromo-4-methoxy-3-methyl-1,3-dihydro-2H-benzo[d]imidazol-2-one

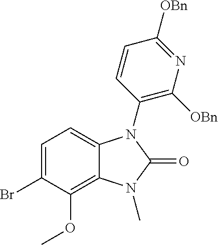 [SMILES: COc1c(Br)ccc2c1n(C)c(=O)n2-c1ccc(OCc2ccccc2)nc1OCc1ccccc1]

To a solution of 1-(2,6-bis(benzyloxy)pyridin-3-yl)-5-bromo-4-methoxy-1,3-dihydro-2H-benzo[d]imidazol-2-one (2.1 g, 3.95 mmol) in DMF (60 mL) were added K₂CO₃ (1.09 g, 7.91 mmol) and CH₃I (1.12 g, 7.91 mmol) at rt for 2 h. The resulting mixture was quenched with water and extracted with EA. The organic layers were washed with brine. The organic layers were concentrated under vacuum. The residue was purified by combi-flash (PE/EA=100:0 to 68:32) to give the product (0.95 g, 44.1%) as brown solid

### Step 5: 1-(2,6-bis(benzyloxy)pyridin-3-yl)-4-methoxy-3-methyl-5-(1,4-dioxa-8-azaspiro[4.5]decan-8-yl)-1,3-dihydro-2H-benzo[d]imidazol-2-one

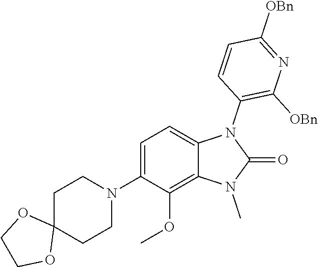 [SMILES: COc1c(N2CCC3(CC2)OCCO3)ccc2c1n(C)c(=O)n2-c1ccc(OCc2ccccc2)nc1OCc1ccccc1]

To a solution of 1-(2,6-bis(benzyloxy)pyridin-3-yl)-5-bromo-4-methoxy-3-methyl-1,3-dihydro-2H-benzo[d]imidazol-2-one (0.9 g, 1.65 mmol) in dry-dioxane (20 mL) were added 1,4-dioxa-8-azaspiro[4.5]decane (354 mg, 2.48 mmol), Pd-PEPPSI-IPentCl 2-methylpyridine (69.3 mg, 0.08 mmol) and Cs₂CO₃ (806 mg, 2.48 mmol) at 100° C. for overnight under N₂ atmosphere. The resulting mixture was concentrated under vacuum. The residue was purified by combi-flash (PE/EA=100:0 to 71:29) to give the product (0.7 g, 70.7%) as light brown semi-solid.

### Step 6: 3-(4-methoxy-3-methyl-2-oxo-5-(1,4-dioxa-8-azaspiro[4.5]decan-8-yl)-2,3-dihydro-1H-benzo[d]imidazol-1-yl)piperidine-2,6-dione

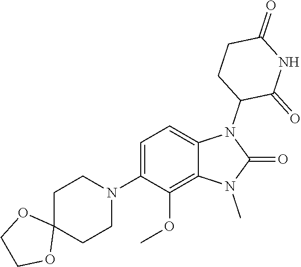 [SMILES: COc1c(N2CCC3(CC2)OCCO3)ccc2c1n(C)c(=O)n2C1CCC(=O)NC1=O]

To a solution of 1-(2,6-bis(benzyloxy)pyridin-3-yl)-4-methoxy-3-methyl-5-(1,4-dioxa-8-azaspiro[4.5]decan-8-yl)-1,3-dihydro-2H-benzo[d]imidazol-2-one (0.7 g, 1.15 mmol) in dry-THF (100 mL) was added Pd/C (0.7 g). The mixture was hydrogenated at room temperature for overnight under hydrogen atmosphere using a hydrogen balloon. The resulting mixture was filtered the filter cake was washed with THF (100 mL). The filtrate was concentrated under vacuum. The residue was without purified to give the product (0.46 g, 92.9%) as light brown solid.

### Step 7: 3-(4-methoxy-3-methyl-2-oxo-5-(4-oxopiperidin-1-yl)-2,3-dihydro-1H-benzo[d]imidazol-1-yl)piperidine-2,6-dione

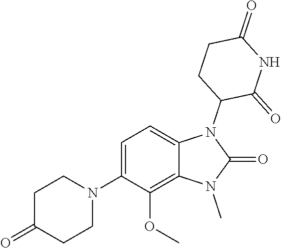 [SMILES: COc1c(N2CCC(=O)CC2)ccc2c1n(C)c(=O)n2C1CCC(=O)NC1=O]

To a solution of 3-(4-methoxy-3-methyl-2-oxo-5-(1,4-dioxa-8-azaspiro[4.5]decan-8-yl)-2,3-dihydro-1H-benzo[d]imidazol-1-yl)piperidine-2,6-dione (0.46 g, 1.07 mmol) in H₂SO₄ (10 mL) at 0° C. The reaction was stirred at rt for 2 h. The resulting mixture was quenched with ice-water (20 mL). The mixture was acidified to PH=7 with NaHCO₃ solution. The resulting mixture was extracted with EA. The organic layers were concentrated under vacuum. The resulting mixture was added ether (15 mL) stirred at rt for 1 h and filtered, the filter cake was washed with ether. The residue was without purified to give the product (344 mg, 83.5%) as yellow green solid. LCMS (ESI) m/e [M+1]⁺=387.

### Step 8: tert-butyl (R)-4-(5-bromo-4-fluoro-1,1-dioxidobenzo[d]isothiazol-2(3H)-yl)-3,3-dimethylpiperidine-1-carboxylate

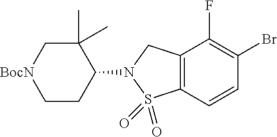 [SMILES: CC(C)(C)OC(=O)N1CC[C@@H](N2Cc3c(ccc(Br)c3F)S2(=O)=O)C(C)(C)C1]

The titled compound was synthesised in the procedure similar to example 16 and further separated by chiral HPLC to give Enantiomer 1 (100% ee); Retention time: 2.87 min. LC-MS (ESI): m/z [M+H]⁺=485.4

Chiral analytical method: Column: CHIRAL PAK IH 4.6*100 mm 5 μm; Mobile phase: A for CO2 and B for MeOH; Gradient (B %) 10% to 50% in 3 min, hold 1 min at 50%; 50% to 10% in 0.1 min. Hold 1.9 min at 10%; HPLC Equipment: HPLC-Agilent, Back pressure: 100 bar; Column temperature: 35° C.

Chiral analytical method: Column: CHIRAL PAK IH 20*250.0 mml, 5 μm; Mobile phase: A for CO2 and B for MeOH; Gradient (B %) 10% to 25% in 3 min, hold 4 min at 25%; Flow Rate: 40 mL/min, Wavelength: UV 220 nm, Prep-HPLC Equipment: Prep-SFC-350; Column temperature: 25° C.

### Step 9: (1R,3S)-3-(3-((2-((4R)-1′-(1-(2,6-dioxopiperidin-3-yl)-4-methoxy-3-methyl-2-oxo-2,3-dihydro-1H-benzo[d]imidazol-5-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl tert-butylcarbamate

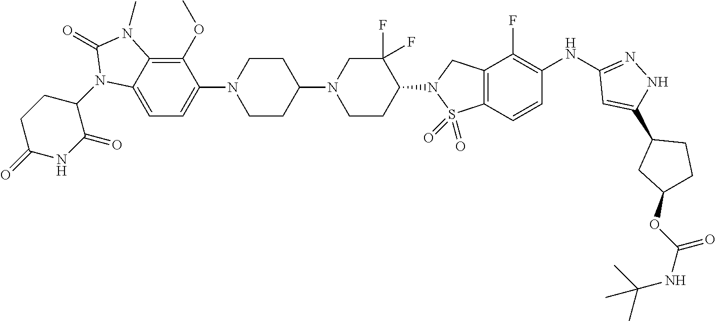 [SMILES: COc1c(N2CCC(N3CC[C@@H](N4Cc5c(ccc(Nc6cc([C@H]7CC[C@@H](OC(=O)NC(C)(C)C)C7)[nH]n6)c5F)S4(=O)=O)C(F)(F)C3)CC2)ccc2c1n(C)c(=O)n2C1CCC(=O)NC1=O]

The titled compound was synthesized in the procedures similar to Example 39. ¹H NMR (500 MHz, DMSO-d₆) δ 11.98 (s, 1H), 11.05 (s, 1H), 8.80 (s, 1H), 8.36-8.30 (s, 1H), 7.61-7.57 (m, 1H), 6.83-6.75 (m, 2H), 6.76-6.66 (m, 1H), 5.87-5.80 (m, 1H), 5.35-5.27 (m, 1H), 5.04-4.97 (m, 1H), 4.79-4.73 (m, 1H), 4.59-4.53 (m, 1H), 4.07-3.98 (m, 1H), 3.93 (s, 3H), 3.50 (s, 3H), 3.45-3.38 (m, 2H), 3.31-3.25 (m, 1H), 3.12-3.01 (m, 2H), 2.87-2.82 (m, 1H), 2.79-2.49 (m, 7H), 2.35-2.22 (m, 1H), 2.17-2.10 (m, 1H), 2.00-1.93 (m, 2H), 1.85-1.79 (m, 3H), 1.78-1.43 (m, 6H), 1.22 (s, 9H). LC-MS (ESI): m/z [M+H]⁺=941.

### Example 106: (1R,3S)-3-(3-((2-((4R)-1-((1-(1-(2,6-dioxopiperidin-3-yl)-3,4-dimethyl-2-oxo-2,3-dihydro-1H-benzo[d]imidazol-5-yl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl tert-butylcarbamate

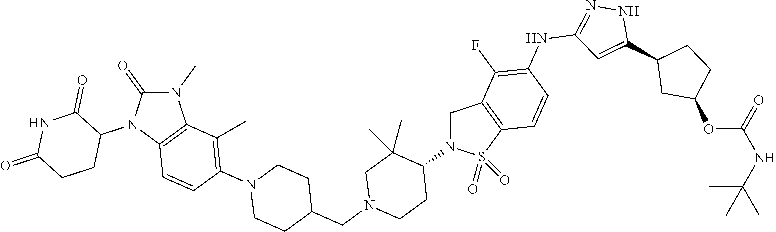 [SMILES: Cc1c(N2CCC(CN3CC[C@@H](N4Cc5c(ccc(Nc6cc([C@H]7CC[C@@H](OC(=O)NC(C)(C)C)C7)[nH]n6)c5F)S4(=O)=O)C(C)(C)C3)CC2)ccc2c1n(C)c(=O)n2C1CCC(=O)NC1=O]

The titled compound was synthesized in the procedures similar to Example 42. ¹H NMR (500 MHz, DMSO) δ 11.98 (s, 1H), 11.06 (s, 1H), 8.77 (s, 1H), 8.30 (s, 1H), 7.54 (d, J=8.6 Hz, 1H), 6.86 (d, J=8.3 Hz, 1H), 6.79-6.76 (m, 2H), 5.83 (s, 1H), 5.32-5.29 (m, 1H), 4.98 (s, 1H), 4.73-4.30 (m, 2H), 3.58 (s, 3H), 3.24-3.21 (m, 1H), 3.07-3.02 (m, 1H), 2.99-2.84 (m, 3H), 2.72-2.69 (m, 2H), 2.64-2.56 (m, 4H), 2.54 (s, 4H), 2.46-2.44 (m, 1H), 2.25-2.10 (m, 3H), 2.03-1.97 (m, 3H), 1.93-1.87 (m, 1H), 1.83-1.81 (m, 3H), 1.73-1.68 (m, 2H), 1.58 (s, 2H), 1.52-1.50 (m, 1H), 1.35-1.25 (m, 1H), 1.21 (s, 9H), 1.13 (s, 3H), 0.94 (s, 3H). LC-MS (ESI): m/z [M+H]⁺=931.87.

### Example 107: (1R,3S)-3-(3-((2-((S)-1-((1-(1-((S)-2,6-dioxopiperidin-3-yl)-4,7-difluoro-3-methyl-2-oxo-2,3-dihydro-1H-benzo[d]imidazol-5-yl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl tert-butylcarbamate

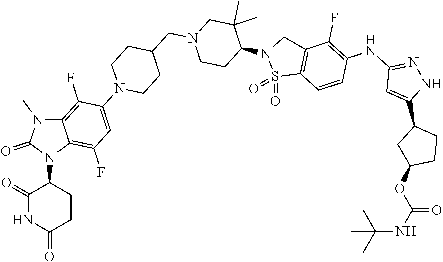 [SMILES: Cn1c(=O)n([C@H]2CCC(=O)NC2=O)c2c(F)cc(N3CCC(CN4CC[C@H](N5Cc6c(ccc(Nc7cc([C@H]8CC[C@@H](OC(=O)NC(C)(C)C)C8)[nH]n7)c6F)S5(=O)=O)C(C)(C)C4)CC3)c(F)c21]

The titled compound was synthesized in the procedures similar to Example 104. LC-MS (ESI): m/z [M+H]⁺=953.65; ¹H NMR (500 MHz, DMSO) δ=11.98 (s, 1H), 11.11 (s, 1H), 8.77 (s, 1H), 8.31 (t, J=7.9, 1H), 7.54 (d, J=8.4, 1H), 6.76 (s, 1H), 6.67 (s, 1H), 5.83 (s, 1H), 5.45 (s, 1H), 4.98 (s, 1H), 4.54 (s, 2H), 3.50 (s, 3H), 3.23 (m, 3H), 3.08-3.03 (m, 1H), 2.94 (m, 2H), 2.58 (m, 4H), 2.47-2.45 (m, 1H), 2.18 (m, 3H), 2.13 (m, 2H), 2.00 (m, 2H), 1.90 (m, 1H), 1.81 (m, 3H), 1.71 (m, 2H), 1.61 (m, 2H), 1.50 (m, 1H), 1.28 (m, 2H), 1.21 (s, 9H), 1.12 (s, 3H), 0.94 (s, 3H).

### Example 108: (1R,3S)-3-(3-((2-((4S)-1-((1-(1-(2,6-dioxopiperidin-3-yl)-3,4-dimethyl-2-oxo-2,3-dihydro-1H-benzo[d]imidazol-5-yl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl tert-butylcarbamate

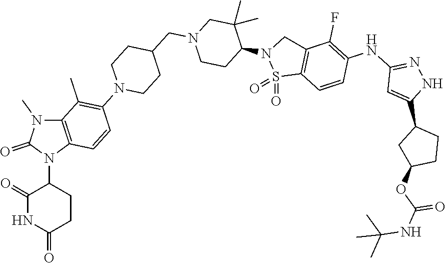 [SMILES: Cc1c(N2CCC(CN3CC[C@H](N4Cc5c(ccc(Nc6cc([C@H]7CC[C@@H](OC(=O)NC(C)(C)C)C7)[nH]n6)c5F)S4(=O)=O)C(C)(C)C3)CC2)ccc2c1n(C)c(=O)n2C1CCC(=O)NC1=O]

The titled compound was synthesized in the procedures similar to Example 42. ¹H NMR (500 MHz, DMSO) δ 11.99 (s, 1H), 11.10 (s, 1H), 8.76 (s, 1H), 8.30 (t, J=8.0 Hz, 1H), 7.51 (d, J=8.6 Hz, 1H), 6.94 (d, J=7.3 Hz, 1H), 6.40 (d, J=13.6 Hz, 1H), 5.83 (s, 1H), 5.38 (s, 1H), 5.06-4.96 (m, 1H), 4.51 (s, 2H), 4.40-4.32 (m, 2H), 3.98-3.86 (m, 2H), 3.72-3.54 (m, 1H), 3.45-3.37 (m, 3H), 3.12-2.85 (m, 4H), 2.61-2.54 (m, 6H), 2.46-2.42 (m, 1H), 2.13-2.16 (m, 2H), 2.14-2.08 (m, 1H), 2.03 (s, 3H), 1.92-1.82 (m, 5H), 1.81-1.66 (m, 4H), 1.64-1.53 (m, 2H), 1.32-1.19 (m, 2H), 1.03 (d, J=6.3 Hz, 6H). LC-MS (ESI): m/z [M+H]⁺=931.4;

### Example 109: (1R,3S)-3-(3-((2-(1-((1-(1-((S)-2,6-dioxopiperidin-3-yl)-8-fluoro-2-oxo-1,2,3,4-tetrahydro-5-oxa-1,2a-diazaacenaphthylen-6-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

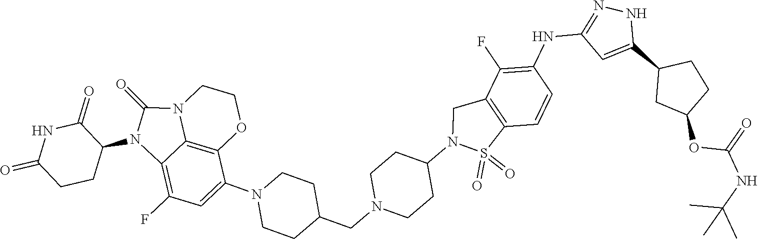 [SMILES: CC(C)(C)NC(=O)O[C@@H]1CC[C@H](c2cc(Nc3ccc4c(c3F)CN(C3CCN(CC5CCN(c6cc(F)c7c8c6OCCn8c(=O)n7[C@H]6CCC(=O)NC6=O)CC5)CC3)S4(=O)=O)n[nH]2)C1]

### Step 1: Step6-bromo-3,4-difluoro-2-nitrophenol

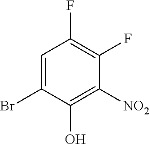 [SMILES: O=[N+]([O-])c1c(O)c(Br)cc(F)c1F]

To a solution of 2-bromo-4,5-difluorophenol (15 g, 71.8 mmol) in DCM (100 mL) were added HNO₃ (6.8 g, 107.7 mmol). The resulting solution was stirred for 1 h at −20° C. The resulting mixture was concentrated under vacuum. The residue was purified by combi-flash (EtOAc/PE=40%) to give the product (14 g, 76.80%) as red solid. LCMS (ESI) m/e [M+1]⁺=254

### Step 2: (2-(6-bromo-3,4-difluoro-2-nitrophenoxy)ethoxy)(tert-butyl)dimethylsilane

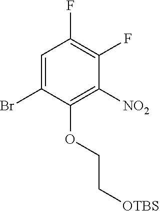 [SMILES: CC(C)(C)[Si](C)(C)OCCOc1c(Br)cc(F)c(F)c1[N+](=O)[O-]]

To a solution of 6-bromo-3,4-difluoro-2-nitrophenol (10 g, 39.3 mmol) in THF (100 mL) were added 2-((tert-butyldimethylsilyl)oxy)ethan-1-ol (10.3 g, 59.1 mmol), PPh₃ (15.5 g, 59.0 mmol), DIAD (2.6 g, 98.3 mmol). The resulting solution was stirred for 16 h at rt. The resulting mixture was concentrated under vacuum. The residue was purified by combi-flash (EtOAc/PE=50%) to give the product (13 g, 80.14%) as red solid. LCMS (ESI) m/e [M+1]⁺=412

### Step 3: 2,6-bis(benzyloxy)-N-(4-bromo-3-(2-((tert-butyldimethylsilyl)oxy)ethoxy)-6-fluoro-2-nitrophenyl)pyridin-3-amine

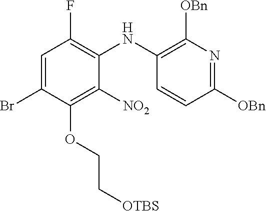 [SMILES: CC(C)(C)[Si](C)(C)OCCOc1c(Br)cc(F)c(Nc2ccc(OCc3ccccc3)nc2OCc2ccccc2)c1[N+](=O)[O-]]

To a solution of 2-bromo-4,5-difluorophenol (20 g, 36.4 mmol) in THF (300 mL) was added LiHMDS (1M in THF, 72.8 mL, 72.8 mmol) dropwise in 30 min at 0° C., the reaction solution was stirred for 1 h at 0° C., then to this was added (2-(6-bromo-3,4-difluoro-2-nitrophenoxy)ethoxy)(tert-butyl)dimethylsilane (15 g, 36.4 mmol) in THF (50 mL) dropwise in 20 min. The resulting solution was stirred for 3 h at rt. The reaction was quenched by the addition of sat·aq. NH₄Cl solution. The resulting solution was extracted with 3×200 mL of EtOAc. The resulting mixture was concentrated under vacuum. The residue was purified by combi-flash (EtOAc/PE=50%) to give the product (17 g, 66.8%) as red oil. LCMS (ESI) m/e [M+1]⁺=698

### Step 4: N1-(2,6-bis(benzyloxy)pyridin-3-yl)-4-bromo-3-(2-((tert-butyldimethylsilyl)oxy)ethoxy)-6-fluorobenzene-1,2-diamine

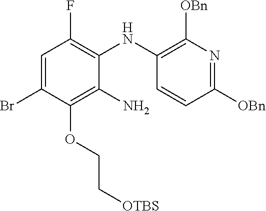 [SMILES: CC(C)(C)[Si](C)(C)OCCOc1c(Br)cc(F)c(Nc2ccc(OCc3ccccc3)nc2OCc2ccccc2)c1N]

To a solution of 2,6-bis(benzyloxy)-N-(4-bromo-3-(2-((tert-butyldimethylsilyl)oxy)ethoxy)-6-fluoro-2-nitrophenyl)pyridin-3-amine (17 g, 24.3 mmol) in MeOH:H2O=4:1 (600 mL) were added Fe (12.6 g, 121.6 mmol) and NH₄Cl (11.9 g, 121.6 mmol). The resulting solution was stirred for 3 h at 80° C. The resulting mixture was filtered, the filter cake was washed with DCM. The filtrate was extracted with 3×300 mL of DCM. The organic layers were concentrated under vacuum. The residue was purified by combi-flash (EtOAc/PE=46%) to give the product (14 g, 86.04%) as red oil. LCMS (ESI) m/e [M+1]⁺=668

### Step 5: 1-(2,6-bis(benzyloxy)pyridin-3-yl)-5-bromo-4-(2-((tert-butyldimethylsilyl)oxy)ethoxy)-7-fluoro-1,3-dihydro-2H-benzo[d]imidazol-2-one

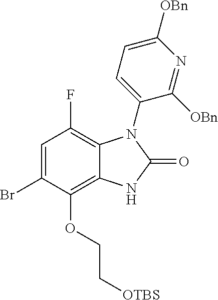 [SMILES: CC(C)(C)[Si](C)(C)OCCOc1c(Br)cc(F)c2c1[nH]c(=O)n2-c1ccc(OCc2ccccc2)nc1OCc1ccccc1]

To a solution of N¹-(2,6-bis(benzyloxy)pyridin-3-yl)-4-bromobenzene-1,2-diamine (13 g, 19.4 mmol) in ACN (100 mL) were added CDI (4.7 g, 29.1 mmol), DIEA (3.8 g, 29.1 mmol). The resulting solution was stirred for 16 h at 100° C. The resulting mixture was concentrated under vacuum. The residue was purified by combi-flash (EtOAc/PE=60%) to give the product (10.3 g, 76.3%) as pink solid. LCMS (ESI) m/e [M+1]⁺=694

### Step 6: 1-(2,6-bis(benzyloxy)pyridin-3-yl)-5-bromo-7-fluoro-4-(2-hydroxyethoxy)-1,3-dihydro-2H-benzo[d]imidazol-2-one

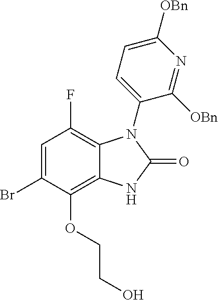 [SMILES: O=c1[nH]c2c(OCCO)c(Br)cc(F)c2n1-c1ccc(OCc2ccccc2)nc1OCc1ccccc1]

To a solution of 1-(2,6-bis(benzyloxy)pyridin-3-yl)-5-bromo-4-(2-((tert-butyldimethylsilyl)oxy)ethoxy)-7-fluoro-1,3-dihydro-2H-benzo[d]imidazol-2-one (10 g, 14.38 mmol) in DMF (100 mL) were added CsF (2.6 g, 28.77 mmol). The resulting solution was stirred for 16 h at 100° C. The resulting solution was extracted with 3×300 mL of EtOAc. The resulting mixture was concentrated under vacuum. The residue was purified by combi-flash (EtOAc/PE=80%) to give the product (7.2 g, 86.3%) as white solid. LCMS (ESI) m/e [M+1]⁺=580

### Step 7: 1-(2,6-bis(benzyloxy)pyridin-3-yl)-6-bromo-8-fluoro-3,4-dihydro-5-oxa-1,2a-diazaacenaphthylen-2(1H)-one

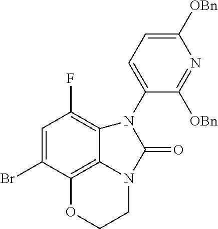 [SMILES: O=c1n2c3c(c(Br)cc(F)c3n1-c1ccc(OCc3ccccc3)nc1OCc1ccccc1)OCC2]

To a solution of 1-(2,6-bis(benzyloxy)pyridin-3-yl)-5-bromo-4-(2-((tert-butyldimethylsilyl)oxy)ethoxy)-7-fluoro-1,3-dihydro-2H-benzo[d]imidazol-2-one (7.1 g, 12.2 mmol) in THF (100 mL) were added DIAD (2.6 g, 18.4 mmol), PPh₃ (2.6 g, 18.4 mmol) at 0° C. The resulting solution was stirred for 16 h at rt. The resulting mixture was concentrated under vacuum. The residue was purified by combi-flash (EtOAc/PE=60%) to give the product (8 g, 99.0%) as white solid. LCMS (ESI) m/e [M+1]⁺=562.

### Step 8: 1-(2,6-bis(benzyloxy)pyridin-3-yl)-6-(4-(dimethoxymethyl)piperidin-1-yl)-8-fluoro-3,4-dihydro-5-oxa-1,2a-diazaacenaphthylen-2(1H)-one

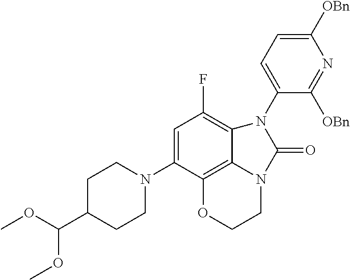 [SMILES: COC(OC)C1CCN(c2cc(F)c3c4c2OCCn4c(=O)n3-c2ccc(OCc3ccccc3)nc2OCc2ccccc2)CC1]

To a solution of 1-(2,6-bis(benzyloxy)pyridin-3-yl)-6-bromo-8-fluoro-3,4-dihydro-5-oxa-1,2a-diazaacenaphthylen-2(1H)-one (2 g, 3.6 mmol) in dioxane (80 mL) were added 4-(dimethoxymethyl)piperidine (858.6 g, 5.4 mmol), Pd-PEPPSI-IPentCl 2-methylpyridine (151.2 mg, 0.18 mmol) and Cs₂CO₃ (2.9 g, 9 mmol). The resulting solution was stirred for 24 h at 100° C. under nitrogen atmosphere. The resulting mixture was concentrated under vacuum. The residue was purified by combi-flash (EtOAc/PE=80%) to give the product (2 g, 87.7%) as yellow solid. LCMS (ESI) m/e [M+1]⁺=641.

### Step 9: 3-(6-(4-(dimethoxymethyl)piperidin-1-yl)-8-fluoro-2-oxo-3,4-dihydro-5-oxa-1,2a-diazaacenaphthylen-1(2H)-yl)piperidine-2,6-dione

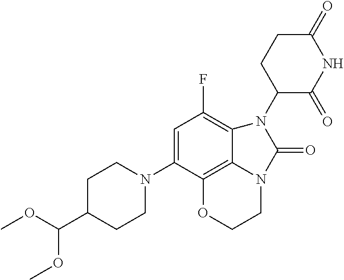 [SMILES: COC(OC)C1CCN(c2cc(F)c3c4c2OCCn4c(=O)n3C2CCC(=O)NC2=O)CC1]

To a solution of 1-(2,6-bis(benzyloxy)pyridin-3-yl)-6-(4-(dimethoxymethyl)piperidin-1-yl)-8-fluoro-3,4-dihydro-5-oxa-1,2a-diazaacenaphthylen-2(1H)-one (2 g, 3.1 mmol) in THF (30 mL) was added Pd/C (2 g, 10% wt). The resulting solution was stirred for 12 h at 50° C. under H₂ atmosphere. After filtration, the filtrate was concentrated under vacuum. The residue was the product (703 mg, 48.7%) as off-white solid. LCMS (ESI) m/e [M+1]⁺=463.

### Step 10: (1R,3S)-3-(3-((2-(1-((1-(1-((S)-2,6-dioxopiperidin-3-yl)-8-fluoro-2-oxo-1,2,3,4-tetrahydro-5-oxa-1,2a-diazaacenaphthylen-6-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

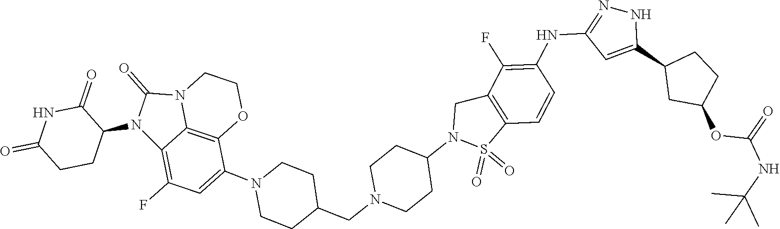 [SMILES: CC(C)(C)NC(=O)O[C@@H]1CC[C@H](c2cc(Nc3ccc4c(c3F)CN(C3CCN(CC5CCN(c6cc(F)c7c8c6OCCn8c(=O)n7[C@H]6CCC(=O)NC6=O)CC5)CC3)S4(=O)=O)n[nH]2)C1]

The titled compound was synthesized in the procedures similar to Example 36. ¹H NMR (500 MHz, DMSO) δ 11.99 (s, 1H), 11.10 (s, 1H), 8.76 (s, 1H), 8.30 (t, J=8.0 Hz, 1H), 7.51 (d, J=8.6 Hz, 1H), 6.94 (d, J=7.3 Hz, 1H), 6.40 (d, J=13.6 Hz, 1H), 5.83 (s, 1H), 5.38 (s, 1H), 5.06-4.96 (m, 1H), 4.51 (s, 2H), 4.40-4.32 (m, 2H), 3.98-3.86 (m, 2H), 3.72-3.54 (m, 1H), 3.45-3.37 (m, 3H), 3.12-2.85 (m, 4H), 2.61-2.54 (m, 6H), 2.46-2.42 (m, 1H), 2.13-2.16 (m, 2H), 2.14-2.08 (m, 1H), 2.03 (s, 3H), 1.92-1.82 (m, 5H), 1.81-1.66 (m, 4H), 1.64-1.53 (m, 2H), 1.32-1.19 (m, 2H), 1.21 (s, 9H). LC-MS (ESI): m/z [M+H]⁺=935.4;

### Example 110: (1R,3S)-3-(3-((2-((R)-1′-(1-((S)-2,6-dioxopiperidin-3-yl)-7-fluoro-3-methyl-2-oxo-2,3-dihydro-1H-benzo[d]imidazol-5-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl tert-butylcarbamate

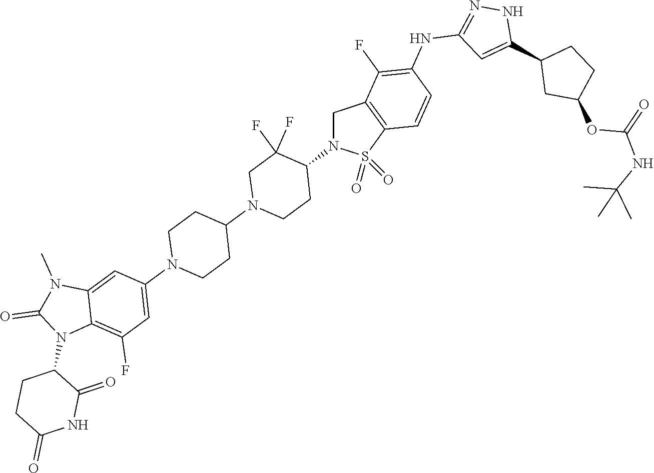 [SMILES: Cn1c(=O)n([C@H]2CCC(=O)NC2=O)c2c(F)cc(N3CCC(N4CC[C@@H](N5Cc6c(ccc(Nc7cc([C@H]8CC[C@@H](OC(=O)NC(C)(C)C)C8)[nH]n7)c6F)S5(=O)=O)C(F)(F)C4)CC3)cc21]

The racemic compound was synthesized in the procedures similar to Example 39 which was further separated by Chiral Prep-HPLC to give Example 110 (100% ee); Retention time: 5.94 min. ¹H NMR (500 MHz, DMSO-d₆) δ 12.00 (s, 1H), 11.07 (s, 1H), 8.82 (s, 1H), 8.33 (s, 1H), 7.58 (d, J=10 Hz, 1H), 6.75-6.55 (m, 2H), 6.61-6.52 (m, 1H), 5.84 (s, 1H), 5.45-5.41 (m, 1H), 5.02-4.95 (m, 1H), 4.78-4.65 (m, 1H), 4.57-4.51 (m, 1H), 4.05-3.94 (m, 1H), 3.78-3.70 (m, 2H), 3.51 (s, 3H), 3.42-3.35 (m, 2H), 3.28-3.21 (m, 3H), 2.75-2.57 (m, 4H), 2.47-2.43 (m, 1H), 2.36-2.01 (m, 4H), 1.91-1.80 (m, 4H), 1.78-1.62 (m, 2H), 1.58-1.51 (m, 3H), 1.30-1.25 (m, 10H). LC-MS (ESI): m/z [M+H]⁺=929.

Chiral analytical method: Amylose-SA, 4.6 mm×250 mm, 5 um; Mobile phase: A for MtBE (0.1% 2M NH3 MeOH) and B for DCM:MeOH (50:50); Gradient: Mobile Phase A: Mobile Phase B=40:60 (v/v); HPLC Equipment: HPLC-Agilent, Back pressure: 100 bar; Column temperature: 35° C.

Chiral Prep-HPLC Condition: Column: Cellulose-SA, 250 mm×20 mm, 5 um; Mobile phase: A for MtBE (0.1% 2M NH3 MeOH) and B for DCM:MeOH (50:50); Gradient: Mobile Phase A: Mobile Phase B=50:50 (v/v); Flow Rate: 20 mL/min, Wave Length: UV 200 nm and 270 nm, Prep-HPLC Equipment: Prep-HPLC-Gilson, Back pressure: 100 bar; Column temperature: 25° C.

### Example 111: (1R,3S)-3-(3-((2-((4R)-1-((1-(1-(2,6-dioxopiperidin-3-yl)-3-methyl-2-oxo-2,3-dihydro-1H-benzo[d]imidazol-5-yl)piperidin-4-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl tert-butylcarbamate

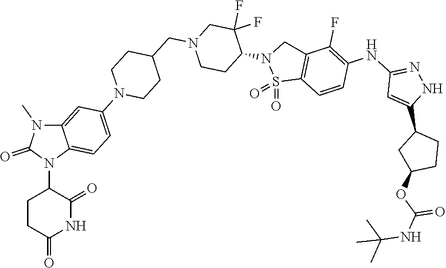 [SMILES: Cn1c(=O)n(C2CCC(=O)NC2=O)c2ccc(N3CCC(CN4CC[C@@H](N5Cc6c(ccc(Nc7cc([C@H]8CC[C@@H](OC(=O)NC(C)(C)C)C8)[nH]n7)c6F)S5(=O)=O)C(F)(F)C4)CC3)cc21]

The titled compound was synthesized in the procedures similar to Example 36. LC-MS (ESI): m/z [M+H]⁺=925.47; ¹H NMR (500 MHz, DMSO) δ=12.01 (s, 1H), 11.07 (s, 1H), 8.83 (s, 1H), 8.32 (s, 1H), 7.58 (d, J=8.6, 1H), 6.95 (s, 1H), 6.85 (s, 1H), 6.76 (s, 1H), 6.66 (s, 1H), 5.84 (s, 1H), 5.30 (d, J=7.1, 1H), 4.98 (s, 1H), 4.75 (d, J=14.3, 1H), 4.54 (d, J=13.4, 1H), 3.99 (m, 1H), 3.59 (m, 2H), 3.32 (m, 3H), 3.18 (m, 1H), 3.06 (m, 1H), 2.94 (m, 1H), 2.93-2.86 (m, 1H), 2.69 (m, 2H), 2.62 (m, 4H), 2.46 (m, 1H), 2.33 (m, 2H), 2.21 (m, 1H), 2.05-1.96 (m, 2H), 1.94-1.87 (m, 1H), 1.82 (m, 3H), 1.71 (m, 3H), 1.59 (s, 1H), 1.28 (m, 2H), 1.21 (s, 9H).

### Example 112: (1R,3S)-3-(3-((2-((4S)-1-((1-(1-(2,6-dioxopiperidin-3-yl)-4-fluoro-3-methyl-2-oxo-2,3-dihydro-1H-benzo[d]imidazol-5-yl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl tert-butylcarbamate

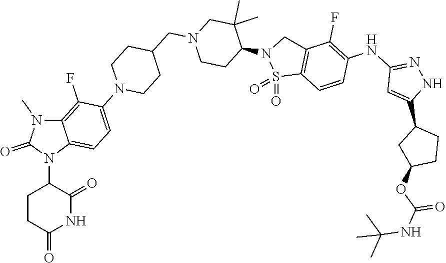 [SMILES: Cn1c(=O)n(C2CCC(=O)NC2=O)c2ccc(N3CCC(CN4CC[C@H](N5Cc6c(ccc(Nc7cc([C@H]8CC[C@@H](OC(=O)NC(C)(C)C)C8)[nH]n7)c6F)S5(=O)=O)C(C)(C)C4)CC3)c(F)c21]

The titled compound was synthesized in the procedures similar to Example 36. LC-MS (ESI): m/z [M+H]⁺=935.56; ¹H NMR (500 MHz, DMSO) δ=11.11 (s, 1H), 8.82 (s, 1H), 8.76 (s, 1H), 8.31 (t, J=7.8, 1H), 7.58 (d, J=8.7, 1H), 6.87 (d, J=8.5, 1H), 6.75 (t, J=7.8, 2H), 5.85 (s, 1H), 5.34 (dd, J=12.8, 5.1, 1H), 4.98 (s, 1H), 4.59 (s, 2H), 3.75 (d, J=9.8, 1H), 3.54 (d, J=10.5, 1H), 3.48 (s, 3H), 3.38 (d, J=12.3, 1H), 3.26 (d, J=10.6, 3H), 3.15-3.04 (m, 3H), 3.01 (m, 1H), 2.89 (m, 1H), 2.75-2.66 (m, 3H), 2.64 (m, 1H), 2.46 (m, 1H), 2.42-2.35 (m, 1H), 2.01 (m, 3H), 1.90 (m, 1H), 1.82 (m, 3H), 1.71 (m, 2H), 1.59 (m, 1H), 1.45 (m, 2H), 1.24 (s, 3H), 1.21 (s, 9H), 1.07 (s, 3H).

### Example 113: (1R,3S)-3-(3-((2-((4S)-1-((1-(1-(2,6-dioxopiperidin-3-yl)-3-methyl-2-oxo-2,3-dihydro-1H-benzo[d]imidazol-5-yl)azetidin-3-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl tert-butylcarbamate

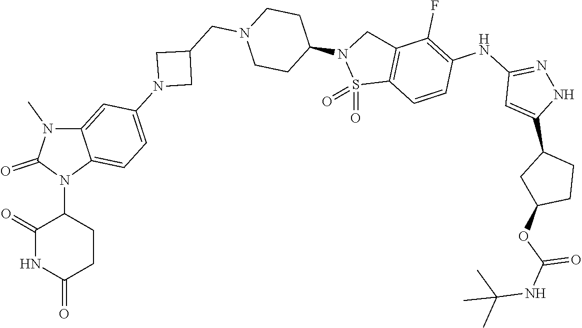

### Step 1: (1-(1-(2,6-dioxopiperidin-3-yl)-3-methyl-2-oxo-2,3-dihydro-1H-benzo[d]imidazol-5-yl)azetidin-3-yl)methyl methanesulfonate

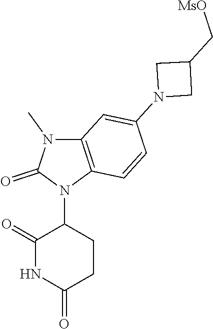 [SMILES: Cn1c(=O)n(C2CCC(=O)NC2=O)c2ccc(N3CC(COS(C)(=O)=O)C3)cc21]

To a solution of 3-(5-(3-(hydroxymethyl)azetidin-1-yl)-3-methyl-2-oxo-2,3-dihydro-1H-benzo[d]imidazol-1-yl)piperidine-2,6-dione (50 mg, 0.14 mmol) in DCM (5 mL), was added TEA (22 mg, 0.22 mmol), methanesulfonyl chloride (31 mg, 0.22 mmol), the reaction mixture was stirred for 2 hours at room temperature. The resulting mixture was diluted with EA, washed with water, then brine, dried over anhydrous Na₂SO₄, then filtered and concentrated under reduced pressure, the residue was purified by silica gel column chromatography, eluting with PE/EA (1:1) to afford the product (50 mg, 81.9% yield). LC-MS (ESI): m/z [M+H]⁺=423.2.

### Step 2: (1R,3S)-3-(3-((2-((4S)-1-((1-(1-(2,6-dioxopiperidin-3-yl)-3-methyl-2-oxo-2,3-dihydro-1H-benzo[d]imidazol-5-yl)azetidin-3-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl tert-butylcarbamate

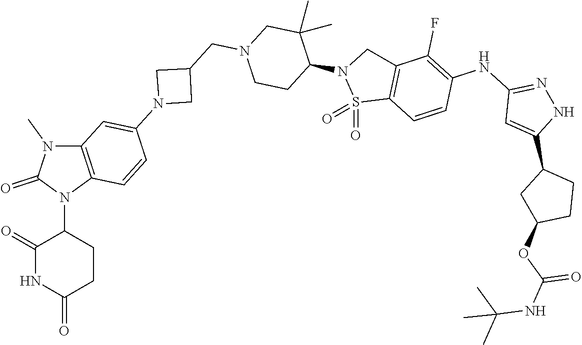 [SMILES: Cn1c(=O)n(C2CCC(=O)NC2=O)c2ccc(N3CC(CN4CC[C@H](N5Cc6c(ccc(Nc7cc([C@H]8CC[C@@H](OC(=O)NC(C)(C)C)C8)[nH]n7)c6F)S5(=O)=O)C(C)(C)C4)C3)cc21]

To a solution of (1R,3S)-3-(3-((2-((S)-3,3-dimethylpiperidin-4-yl)-4-fluoro-1,1-dioxido-2,3-dihydrobenzo[d]isothiazol-5-yl)amino)-1H-pyrazol-5-yl)cyclopentyl tert-butylcarbamate (30 mg, 0.05 mmol), (1-(1-(2,6-dioxopiperidin-3-yl)-3-methyl-2-oxo-2,3-dihydro-1H-benzo[d]imidazol-5-yl)azetidin-3-yl)methyl methanesulfonate (29.3 mg, 0.07 mmol), in ACN (4 mL), was added DIEA (13 mg, 0.1 mmol), potassium iodide (83 mg, 0.5 mmol), the reaction mixture was stirred under nitrogen atmosphere at 80° C. for 16 h. The resulting mixture was diluted with EA, washed with water, then brine, dried over anhydrous Na₂SO₄, then filtered and concentrated under reduced pressure. The resulting mixture was concentrated, and the residue was purified by column chromatography on silica gel, eluting with DCM/MeOH (10:1) to afford the product (30 mg, 63.8%). The residue 30 mg was purified by prep HPLC (Waters XSelect C18: RD-CO-094 column, eluting with a gradient of acetonitrile/water containing 0.1% FA, 20%-45%) to afford the product (20 mg, 66.6%).

¹H NMR (500 MHz, DMSO) δ 11.98 (s, 1H), 11.04 (s, 1H), 8.77 (s, 1H), 8.31 (s, 1H), 7.54 (d, J=8.6 Hz, 1H), 6.90 (d, J=8.3 Hz, 1H), 6.76 (s, 1H), 6.31 (s, 1H), 6.10 (d, J=8.0 Hz, 1H), 5.84 (s, 1H), 5.27 (dd, J=12.7, 5.4 Hz, 1H), 5.06-4.96 (m, 1H), 4.54 (s, 2H), 3.91 (q, J=7.0 Hz, 2H), 3.47-3.39 (m, 2H), 3.28 (s, 3H), 3.22 (dd, J=12.1, 3.5 Hz, 1H), 3.10-3.00 (m, 1H), 2.98-2.85 (m, 3H), 2.66-2.58 (m, 4H), 2.20-2.11 (m, 1H), 2.08-1.96 (m, 3H), 1.94-1.86 (m, 2H), 1.77-1.67 (m, 2H), 1.64-1.55 (m, 1H), 1.54-1.47 (m, 1H), 1.21 (s, 9H), 1.11 (s, 3H), 0.94 (s, 3H). LC-MS (ESI): m/z [M+H]⁺=889.4;

### Example 114: (1S,3R)-3-(3-((2-(1-((1-(4-((R)-2,6-dioxopiperidin-3-yl)-3,5-difluorophenyl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl propylcarbamate

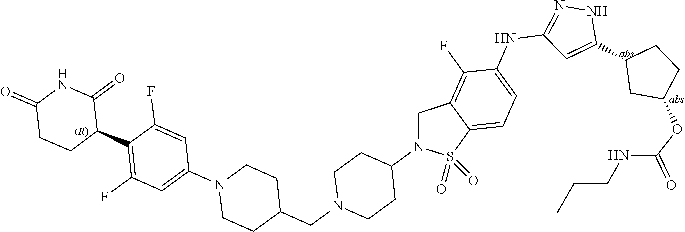

### Step 1: (1S,3R)-3-(3-((4-fluoro-1,1-dioxido-2-(piperidin-4-yl)-2,3-dihydrobenzo[d]isothiazol-5-yl)amino)-1H-pyrazol-5-yl)cyclopentyl propylcarbamate

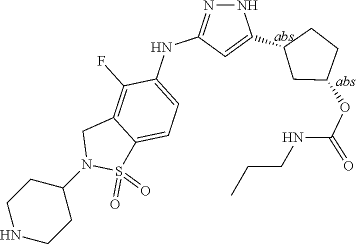 [SMILES: CCCNC(=O)O[C@H]1CC[C@@H](c2cc(Nc3ccc4c(c3F)CN(C3CCNCC3)S4(=O)=O)n[nH]2)C1]

The titled compound was synthesized in the procedures similar to Example 1. LC-MS (ESI): m/z [M+H]⁺=521.4

### Step 2: (1S,3R)-3-(3-((2-(1-((1-(4-((R)-2,6-dioxopiperidin-3-yl)-3,5-difluorophenyl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentylpropylcarbamate

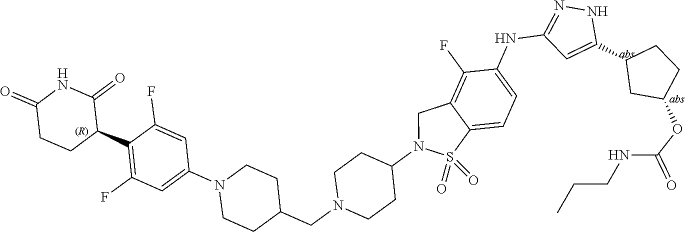

The titled compound was synthesized in the procedures similar to Example 1. ¹H NMR (500 MHz, DMSO-d₆) δ 11.99 (s, 1H), 10.86 (s, 1H), 8.76 (s, 1H), 8.29 (s, 1H), 7.51 (d, J=5 Hz, 1H), 7.15-7.10 (m, 2H), 6.61 (d, J=5 Hz, 1H), 5.83 (s, 1H), 5.02-4.95 (m, 1H), 4.51 (s, 1H), 4.18-4.05 (m, 1H), 3.78-3.70 (m, 2H), 3.46-3.38 (m, 1H), 3.11-3.02 (m, 1H), 2.95-2.86 (m, 4H), 2.82-2.69 (m, 4H), 2.47-2.43 (m, 1H), 2.21-2.11 (m, 2H), 2.10-1.99 (m, 4H), 1.98-1.82 (m, 6H), 1.80-1.65 (m, 5H), 1.64-1.56 (m, 1H), 1.42-1.31 (m, 2H), 1.30-1.21 (m, 1H), 1.20-1.09 (m, 2H), 0.89-0.78 (m, 3H). LC-MS (ESI): m/z [M+H]⁺=841.

### Example 115: (1S,3R)-3-(3-((2-(1-((1-(1-(2,6-dioxopiperidin-3-yl)-3-methyl-2-oxo-2,3-dihydro-1H-benzo[d]imidazol-5-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl propylcarbamate

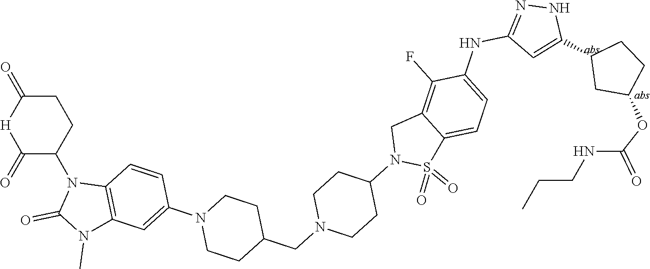

The titled compound was synthesized in the procedures similar to Example 36. ¹H NMR (500 MHz, DMSO-d₆) δ 12.01 (s, 1H), 11.06 (s, 1H), 8.76 (s, 1H), 8.28 (s, 1H), 7.51 (d, J=5 Hz, 1H), 7.04 (t, J=5 Hz, 1H), 7.00 (d, J=5 Hz, 1H), 6.82-6.75 (m, 1H), 6.72-6.66 (m, 1H), 5.83 (s, 1H), 5.37-5.29 (m, 1H), 5.04-4.96 (m, 1H), 4.51 (s, 2H), 3.59-3.47 (m, 2H), 3.46-3.38 (m, 1H), 3.27 (s, 3H), 3.11-3.02 (m, 1H), 2.95-2.81 (m, 5H), 2.80-2.69 (m, 1H), 2.67-2.56 (m, 3H), 2.47-2.43 (m, 1H), 2.21-2.15 (m, 2H), 2.07-1.95 (m, 4H), 1.93-1.66 (m, 9H), 1.64-1.56 (m, 2H), 1.44-1.31 (m, 2H), 1.30-1.18 (m, 2H), 0.86-0.76 (m, 3H). LC-MS (ESI): m/z [M+H]⁺=875.

### Example 116: (1S,3R)-3-(3-((2-(1-((1-(3-(2,6-dioxopiperidin-3-yl)-2-oxo-2,3-dihydrobenzo[d]oxazol-6-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl propylcarbamate

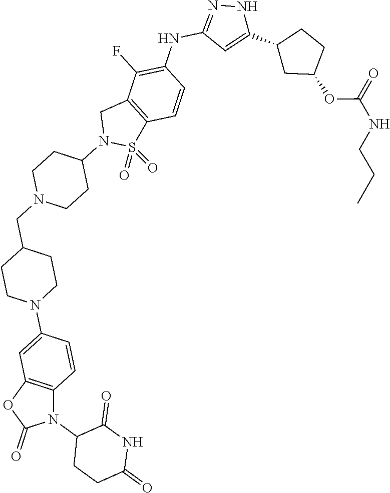 [SMILES: CCCNC(=O)O[C@H]1CC[C@@H](c2cc(Nc3ccc4c(c3F)CN(C3CCN(CC5CCN(c6ccc7c(c6)oc(=O)n7C6CCC(=O)NC6=O)CC5)CC3)S4(=O)=O)n[nH]2)C1]

The titled compound was synthesized in the procedures similar to Example 36. ¹H NMR (500 MHz, DMSO-d₆) δ 12.00 (s, 1H), 11.17 (s, 1H), 8.76 (s, 1H), 8.28 (s, 1H), 7.51 (d, J=5 Hz, 1H), 7.18-7.04 (m, 3H), 6.80-6.75 (m, 1H), 5.83 (s, 1H), 5.37-5.29 (m, 1H), 5.04-4.96 (m, 1H), 4.52 (s, 2H), 3.59-3.47 (m, 2H), 3.46-3.38 (m, 1H), 3.11-3.02 (m, 1H), 2.95-2.81 (m, 5H), 2.70-2.56 (m, 4H), 2.47-2.43 (m, 1H), 2.21-2.15 (m, 3H), 2.07-1.95 (m, 3H), 1.93-1.66 (m, 9H), 1.64-1.56 (m, 2H), 1.44-1.31 (m, 2H), 1.30-1.19 (m, 2H), 0.86-0.76 (m, 3H). LC-MS (ESI): m/z [M+H]⁺=862.

### Example 117: (1R,3S)-3-(3-((2-(1-((1-(1-(2,6-dioxopiperidin-3-yl)-3-methyl-2-oxo-2,3-dihydro-1H-benzo[d]imidazol-5-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl propylcarbamate

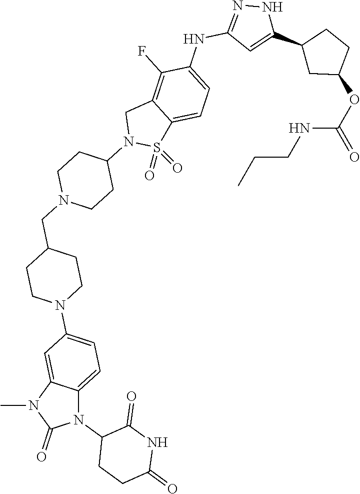 [SMILES: CCCNC(=O)O[C@@H]1CC[C@H](c2cc(Nc3ccc4c(c3F)CN(C3CCN(CC5CCN(c6ccc7c(c6)n(C)c(=O)n7C6CCC(=O)NC6=O)CC5)CC3)S4(=O)=O)n[nH]2)C1]

The titled compound was synthesized in the procedures similar to Example 36. ¹H NMR (500 MHz, DMSO-d₆) δ 12.00 (s, 1H), 11.06 (s, 1H), 8.76 (s, 1H), 8.28 (s, 1H), 7.51 (d, J=10 Hz, 1H), 7.04 (d, J=5 Hz, 1H), 6.92 (d, J=10 Hz, 1H), 6.84-6.81 (m, 1H), 6.65-6.61 (m, 1H), 5.83 (s, 1H), 5.32-5.25 (m, 1H), 5.05-4.96 (m, 1H), 4.52 (s, 2H), 3.62-3.55 (m, 2H), 3.48-3.44 (m, 1H), 3.30 (s, 3H), 3.10-3.01 (m, 1H), 2.96-2.84 (m, 5H), 2.73-2.57 (m, 4H), 2.47-2.43 (m, 1H), 2.22-2.16 (m, 2H), 2.08-1.95 (m, 4H), 1.94-1.76 (m, 7H), 1.75-1.67 (m, 2H), 1.66-1.56 (m, 2H), 1.42-1.33 (m, 2H), 1.30-1.20 (m, 2H), 0.85-0.76 (m, 3H). LC-MS (ESI): m/z [M+H]⁺=875.

### Example 118: (1R,3S)-3-(3-((2-(1-((1-(4-((R)-2,6-dioxopiperidin-3-yl)-3,5-difluorophenyl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

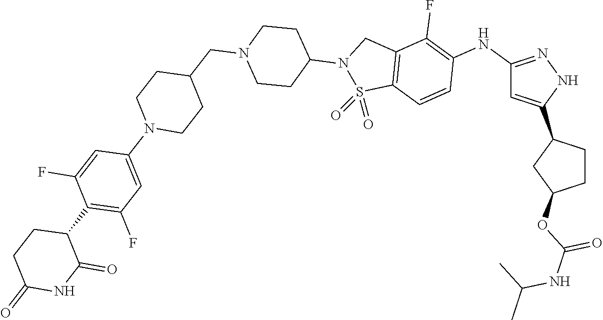 [SMILES: CC(C)NC(=O)O[C@@H]1CC[C@H](c2cc(Nc3ccc4c(c3F)CN(C3CCN(CC5CCN(c6cc(F)c([C@H]7CCC(=O)NC7=O)c(F)c6)CC5)CC3)S4(=O)=O)n[nH]2)C1]

The titled compound was synthesized in the procedures similar to Example 1 in a racemic form, which was further separated by Chiral Prep-HPLC to give Example 118 (100% ee); Retention time: 5.806 min. ¹H NMR (500 MHz, DMSO) δ 11.99 (s, 1H), 10.86 (s, 1H), 8.77 (s, 1H), 8.29 (s, 1H), 7.51 (d, J=8.6 Hz, 1H), 6.94 (d, J=7.3 Hz, 1H), 6.61 (d, J=12.9 Hz, 2H), 5.83 (s, 1H), 5.06-4.96 (m, 1H), 4.52 (s, 2H), 4.04 (dd, J=12.5, 4.9 Hz, 1H), 3.75 (d, J=11.4 Hz, 2H), 3.65-3.53 (m, 1H), 3.47-3.37 (m, 1H), 3.29 (s, 1H), 3.13-3.01 (m, 1H), 2.93-2.86 (m, 2H), 2.83-2.68 (m, 3H), 2.48-2.42 (m, 1H), 2.20-2.14 (m, 2H), 2.08 (dd, J=12.9, 3.6 Hz, 1H), 2.06-1.99 (m, 3H), 1.98-1.80 (m, 6H), 1.80-1.65 (m, 5H), 1.63-1.55 (m, 1H), 1.2-1.07 (m, 2H), 1.03 (d, J=6.4 Hz, 6H). LC-MS (ESI): m/z [M+H]⁺=841.4;

Chiral analytical method: Column: ICHIRALPAK SA 4.6*150 mm 5 μm; Mobile phase: A for MtBE (0.1%2M NH3 MeOH) (%) and B for MeOH:DCM=50:50(%); Gradient: Mobile Phase A: Mobile Phase B=30:70 (v/v); HPLC Equipment: HPLC-Agilent, Back pressure: 100 bar; Column temperature: 35° C.

Chiral Prep-HPLC Condition: CHIRALPAK SA 250*20.0 mml, 5 μm; Mobile phase: A for MtBE (0.1%2M NH3 MeOH) (%) and B for MeOH:DCM=50:50; Gradient: Mobile Phase A: Mobile Phase B=30:70 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 119: (1R,3S)-3-(3-((2-(1-((1-(4-((R)-2,6-dioxopiperidin-3-yl)-3,5-difluorophenyl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

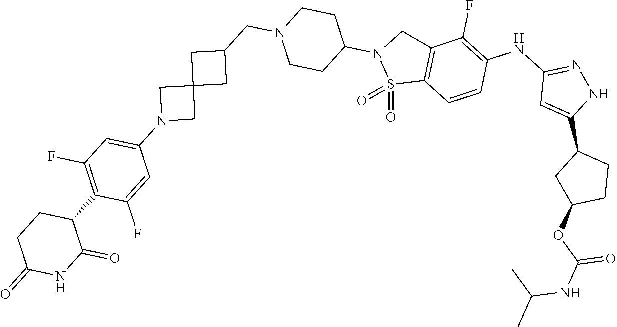 [SMILES: CC(C)NC(=O)O[C@@H]1CC[C@H](c2cc(Nc3ccc4c(c3F)CN(C3CCN(CC5CC6(C5)CN(c5cc(F)c([C@H]7CCC(=O)NC7=O)c(F)c5)C6)CC3)S4(=O)=O)n[nH]2)C1]

The titled compound was synthesized in the procedures similar to Example 1. ¹H NMR (500 MHz, DMSO) δ 11.99 (s, 1H), 10.85 (s, 1H), 8.76 (s, 1H), 8.27 (s, 1H), 7.50 (d, J=8.6 Hz, 1H), 6.94 (d, J=7.6 Hz, 1H), 6.08 (d, J=11.1 Hz, 2H), 5.83 (s, 1H), 5.06-4.96 (m, 1H), 4.50 (s, 2H), 4.02 (dd, J=12.5, 4.9 Hz, 1H), 3.85 (s, 2H), 3.73 (s, 2H), 3.63-3.54 (m, 1H), 3.44-3.37 (m, 2H), 3.13-2.99 (m, 1H), 2.85 (d, J=11.2 Hz, 2H), 2.82-2.71 (m, 1H), 2.47-2.41 (m, 1H), 2.39-2.23 (m, 5H), 2.16-1.98 (m, 4H), 1.97-1.78 (m, 8H), 1.75-1.55 (m, 3H), 1.03 (d, J=6.4 Hz, 6H). LC-MS (ESI): m/z [M+H]⁺=853.4;

### Example 120 and 121: (1R,3S)-3-(3-((2-(1-(((S)-1-(4-((R)-2,6-dioxopiperidin-3-yl)-3,5-difluorophenyl)-3,3-dimethylpiperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate and (1R,3S)-3-(3-((2-(1-(((R)-1-(4-((R)-2,6-dioxopiperidin-3-yl)-3,5-difluorophenyl)-3,3-dimethylpiperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

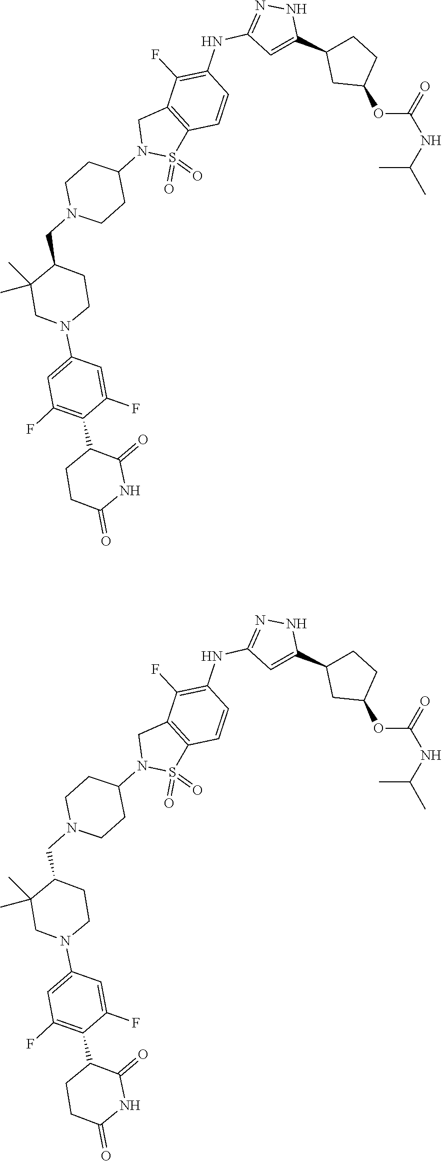

### Step 1: 1-(4-(2,6-dioxopiperidin-3-yl)-3,5-difluorophenyl)-3,3-dimethylpiperidine-4-carbaldehyde

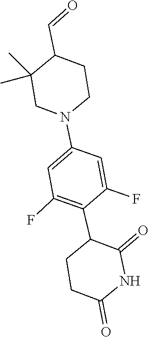 [SMILES: CC1(C)CN(c2cc(F)c(C3CCC(=O)NC3=O)c(F)c2)CCC1C=O]

### Step2: (1R,3S)-3-(3-((2-(1-(((S)-1-(4-((R)-2,6-dioxopiperidin-3-yl)-3,5-difluorophenyl)-3,3-dimethylpiperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate and (1R,3S)-3-(3-((2-(1-(((R)-1-(4-((R)-2,6-dioxopiperidin-3-yl)-3,5-difluorophenyl)-3,3-dimethylpiperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

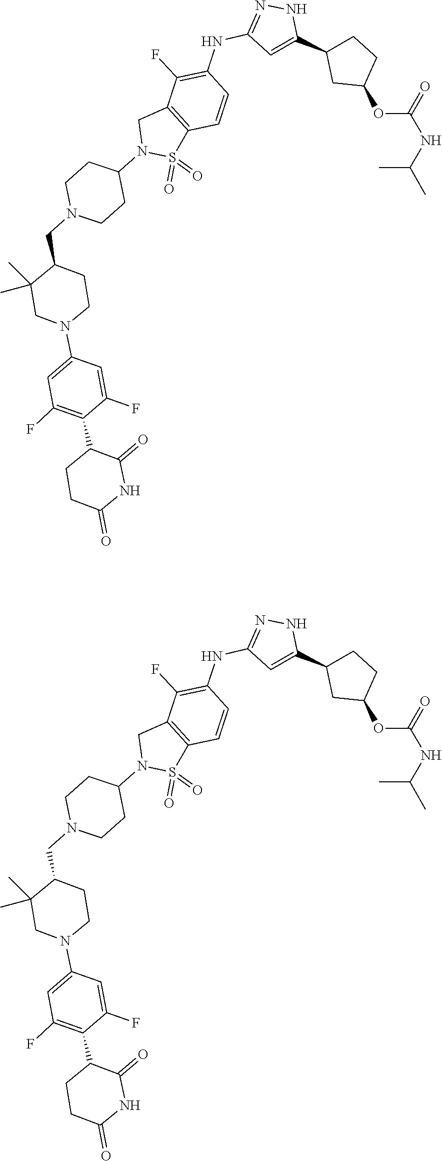

The racemic compound was synthesized in the procedures similar to Example 1, which was further separated by Chiral Prep-HPLC to afford the desired product:

Enantiomer 1 (Example 120, 100% ee); Retention time: 7.74 min. ¹H NMR (500 MHz, DMSO-d₆) δ 11.99 (s, 1H), 10.86 (s, 1H), 8.77 (s, 1H), 8.32 (t, J=5 Hz, 1H), 7.51 (d, J=10 Hz, 1H), 6.99-6.91 (m, 1H), 6.65-6.56 (m, 2H), 5.83 (s, 1H), 5.04-4.96 (m, 1H), 4.51 (s, 2H), 4.06-3.99 (m, 1H), 3.76-3.68 (m, 1H), 3.63-3.53 (m, 1H), 3.47-3.40 (m, 1H), 3.39-3.35 (m, 1H), 3.11-3.03 (m, 1H), 3.01-2.95 (m, 1H), 2.86-2.72 (m, 3H), 2.68-2.59 (m, 1H), 2.48-2.42 (m, 2H), 2.34-2.26 (m, 1H), 2.21-2.11 (m, 2H), 2.10-1.98 (m, 3H), 1.98-1.77 (m, 6H), 1.77-1.55 (m, 3H), 1.48-1.28 (m, 2H), 1.06-0.99 (m, 6H), 0.98 (s, 3H), 0.82 (s, 3H). LC-MS (ESI): m/z [M+H]⁺=869.

Enantiomer 2 (Example 121, 100% ee); Retention time: 8.85 min. ¹H NMR (500 MHz, DMSO-d₆) δ 11.99 (s, 1H), 10.86 (s, 1H), 8.77 (s, 1H), 8.32 (t, J=5 Hz, 1H), 7.51 (d, J=10 Hz, 1H), 6.99-6.91 (m, 1H), 6.65-6.56 (m, 2H), 5.83 (s, 1H), 5.04-4.96 (m, 1H), 4.51 (s, 2H), 4.06-3.99 (m, 1H), 3.76-3.68 (m, 1H), 3.63-3.53 (m, 1H), 3.47-3.40 (m, 1H), 3.39-3.35 (m, 1H), 3.11-3.03 (m, 1H), 3.01-2.95 (m, 1H), 2.86-2.72 (m, 3H), 2.68-2.59 (m, 1H), 2.48-2.42 (m, 2H), 2.34-2.26 (m, 1H), 2.21-2.11 (m, 2H), 2.10-1.98 (m, 3H), 1.98-1.77 (m, 6H), 1.77-1.55 (m, 3H), 1.48-1.28 (m, 2H), 1.06-0.99 (m, 6H), 0.98 (s, 3H), 0.82 (s, 3H). LC-MS (ESI): m/z [M+H]⁺=869.

Chiral analytical method: Amylose-SA, 4.6 mm×250 mm, 5 um; Mobile phase: A for MtBE (0.1% 2M NH3 MeOH) and B for DCM:MeOH (50:50); Gradient: Mobile Phase A: Mobile Phase B=50:50 (v/v); HPLC Equipment: HPLC-Agilent, Back pressure: 100 bar; Column temperature: 35° C.

Chiral Prep-HPLC Condition: Column: Amylose-SA, 250 mm×20 mm, 5 um; Mobile phase: A for MtBE (0.1% 2M NH3 MeOH) and B for DCM:MeOH (50:50); Gradient: Mobile Phase A: Mobile Phase B=50:50 (v/v); Flow Rate: 20 mL/min, Wave Length: UV 200 nm and 270 nm, Prep-HPLC Equipment: Prep-HPLC-Gilson, Back pressure: 100 bar; Column temperature: 25° C.

### Example 122: (1R,3S)-3-(3-((2-(1′-(1-(2,6-dioxopiperidin-3-yl)-4-fluoro-3-methyl-2-oxo-2,3-dihydro-1H-benzo[d]imidazol-5-yl)-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

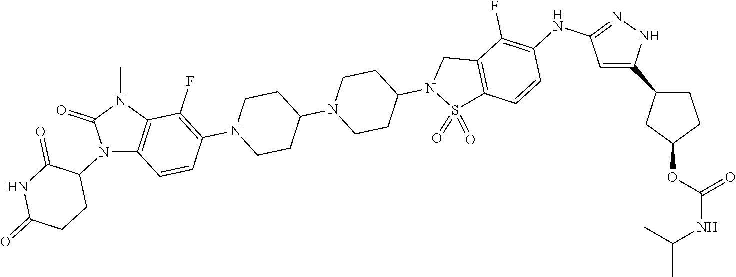 [SMILES: CC(C)NC(=O)O[C@@H]1CC[C@H](c2cc(Nc3ccc4c(c3F)CN(C3CCN(C5CCN(c6ccc7c(c6F)n(C)c(=O)n7C6CCC(=O)NC6=O)CC5)CC3)S4(=O)=O)n[nH]2)C1]

The titled compound was synthesized in the procedures similar to Example 39. ¹H NMR (500 MHz, DMSO) δ 12.00 (s, 1H), 11.10 (s, 1H), 8.76 (s, 1H), 8.28 (s, 1H), 7.51 (d, J=8.6 Hz, 1H), 6.94 (d, J=7.4 Hz, 1H), 6.85 (d, J=8.5 Hz, 1H), 6.74 (t, J=8.2 Hz, 1H), 5.83 (s, 1H), 5.33 (dd, J=12.9, 5.3 Hz, 1H), 5.06-4.96 (m, 1H), 4.52 (s, 2H), 3.63-3.53 (m, 1H), 3.50-3.43 (m, 3H), 3.44-3.39 (m, 1H), 3.29-3.25 (m, 2H), 3.13-2.96 (m, 3H), 2.92-2.84 (m, 1H), 2.74-2.62 (m, 4H), 2.48-2.40 (m, 2H), 2.38-2.31 (m, 2H), 2.10-1.79 (m, 10H), 1.78-1.55 (m, 5H), 1.03 (d, J=6.4 Hz, 6H). LC-MS (ESI): m/z [M+H]⁺=879.4;

### Example 123: (1R,3S)-3-(3-((2-(1-((1-(1-(2,6-dioxopiperidin-3-yl)-4-fluoro-3-methyl-2-oxo-2,3-dihydro-1H-benzo[d]imidazol-5-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

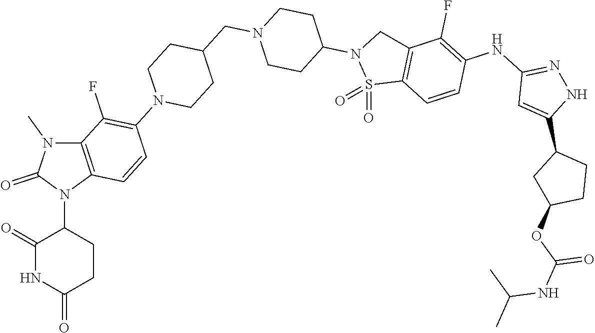 [SMILES: CC(C)NC(=O)O[C@@H]1CC[C@H](c2cc(Nc3ccc4c(c3F)CN(C3CCN(CC5CCN(c6ccc7c(c6F)n(C)c(=O)n7C6CCC(=O)NC6=O)CC5)CC3)S4(=O)=O)n[nH]2)C1]

The titled compound was synthesized in the procedures similar to Example 36. ¹H NMR (500 MHz, DMSO) δ 11.99 (s, 1H), 11.10 (s, 1H), 8.76 (s, 1H), 8.29 (s, 1H), 7.51 (d, J=8.6 Hz, 1H), 5.83 (s, 1H), 5.33 (dd, J=12.7, 5.3 Hz, 1H), 5.06-4.96 (m, 1H), 4.52 (s, 2H), 3.63-3.53 (m, 1H), 3.47 (s 3H), 3.44-3.39 (m, 1H), 3.23 (d, J=11.3 Hz, 2H), 3.12-3.00 (m, 1H), 2.94-2.84 (m, 3H), 2.75-2.56 (m, 5H), 2.47-2.42 (m, 1H), 2.20 (d, J=6.8 Hz, 2H), 2.07-1.97 (m, 4H), 1.95-1.53 (m, 12H), 1.34-1.23 (m, 2H), 1.03 (d, J=6.4 Hz, 6H). LC-MS (ESI): m/z [M+H]⁺=893.4;

### Example 124: (1R,3S)-3-(3-((2-(1-((1-(1-(2,6-dioxopiperidin-3-yl)-3-methyl-2-oxo-2,3-dihydro-1H-benzo[d]imidazol-5-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

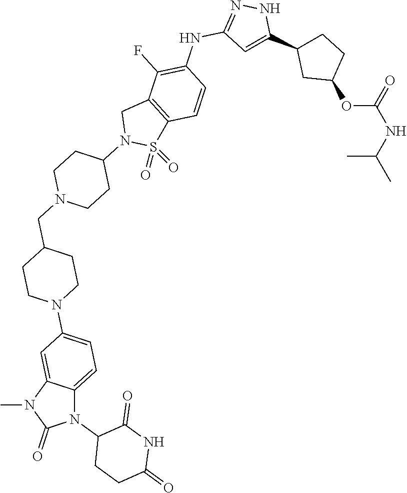 [SMILES: CC(C)NC(=O)O[C@@H]1CC[C@H](c2cc(Nc3ccc4c(c3F)CN(C3CCN(CC5CCN(c6ccc7c(c6)n(C)c(=O)n7C6CCC(=O)NC6=O)CC5)CC3)S4(=O)=O)n[nH]2)C1]

The titled compound was synthesized in the procedures similar to Example 36. ¹H NMR (500 MHz, DMSO-d₆) δ 12.00 (s, 1H), 11.06 (s, 1H), 8.76 (s, 1H), 8.28 (s, 1H), 7.51 (d, J=10 Hz, 1H), 6.98-6.89 (m, 2H), 6.84-6.80 (m, 1H), 6.64-6.61 (m, 1H), 5.83 (s, 1H), 5.34-5.25 (m, 1H), 5.07-4.96 (m, 1H), 4.52 (s, 2H), 3.62-3.53 (m, 3H), 3.48-3.44 (m, 1H), 3.30 (s, 3H), 3.12-3.01 (m, 1H), 2.96-2.80 (m, 3H), 2.73-2.57 (m, 4H), 2.47-2.43 (m, 1H), 2.22-2.15 (m, 2H), 2.08-1.95 (m, 4H), 1.94-1.76 (m, 7H), 1.75-1.67 (m, 2H), 1.66-1.56 (m, 2H), 1.32-1.20 (m, 2H), 1.10-0.96 (m, 6H). LC-MS (ESI): m/z [M+H]⁺=875.

### Example 125: (1R,3S)-3-(3-((2-(1′-(3-(2,6-dioxopiperidin-3-yl)-2-oxo-2,3-dihydroben-zo[d]oxazol-7-yl)-[1,4′-bipiperidin]-4-yl)-4-fluoro-1,1-dioxido-2,3-dihydrobenzo[d]iso-thiazol-5-yl)amino)-1H-pyrazol-5-yl)cyclopentyl isopropylcarbamate

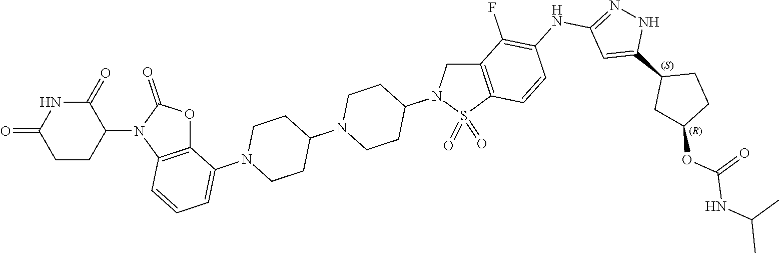

### Step 1: 3-(2,6-bis(benzyloxy)pyridin-3-yl)-7-(1,4-dioxa-8-azaspiro[4.5]decan-8-yl)benzo[d]oxazol-2(3H)-one

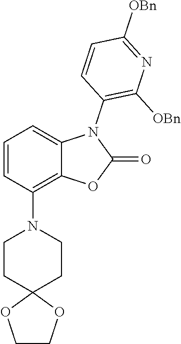 [SMILES: O=c1oc2c(N3CCC4(CC3)OCCO4)cccc2n1-c1ccc(OCc2ccccc2)nc1OCc1ccccc1]

To a solution of 3-(2,6-bis(benzyloxy)pyridin-3-yl)-7-bromobenzo[d]oxazol-2(3H)-one (PH-BGY-H-A517-11) (2.5 g, 4.98 mmol) in Dioxane (100 mL) were added 1,4-dioxa-8-azaspiro[4.5]decane (1.07 g, 7.47 mmol), Pd-PEPPSI-IpentCl (209 mg, 0.25 mmol) and Cs₂CO₃ (4.87 g, 14.94 mmol) at 100° C. for overnight under N₂ atmosphere. The reaction was concentrated under reduced pressure. The crude was purified by combine-flash PE:EA=100:0 to 65:35 to give the product (1.9 g, 67.6%) as light yellow solid.

### Step 2: 3-(2-oxo-7-(1,4-dioxa-8-azaspiro[4.5]decan-8-yl)benzo[d]oxazol-3(2H)-yl)piperidine-2,6-dione

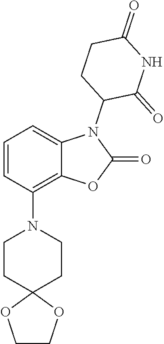 [SMILES: O=C1CCC(n2c(=O)oc3c(N4CCC5(CC4)OCCO5)cccc32)C(=O)N1]

To a solution of 3-(2,6-bis(benzyloxy)pyridin-3-yl)-7-(1,4-dioxa-8-azaspiro[4.5]decan-8-yl)benzo[d]oxazol-2(3H)-one (1.9 g, 3.36 mmol) in THF (100 mL) was added Pd/C (1.9 g) at 50° C. for overnight under H₂ atmosphere. The reaction was filtered. The filtrate was concentrated under vacuum. The residue was without purified to give the product (1 g, 76.9%) as yellow semi-solid.

### Step 3: 3-(2-oxo-7-(4-oxopiperidin-1-yl)benzo[d]oxazol-3(2H)-yl)piperidine-2,6-dione

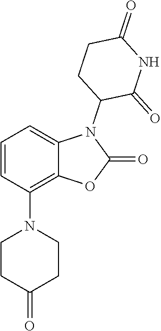 [SMILES: O=C1CCN(c2cccc3c2oc(=O)n3C2CCC(=O)NC2=O)CC1]

To a solution of 3-(2-oxo-7-(1,4-dioxa-8-azaspiro[4.5]decan-8-yl)benzo[d]oxazol-3(2H)-yl)piperidine-2,6-dione (1 g, 2.58 mmol) in FA (30 mL) at 70° C. for 3 h. The reaction was concentrated under vacuum. The resulting solution was quenched with Et₂O (25 mL) stirred at rt for 2 h. The resulting mixture was filtered. The filtrate cake was concentrated under vacuum. The residue was purified by combi-flash PE:EA=100:0 to 70:30 to give the product (670.3 mg, 75.6%) as yellow green solid. LCMS (ESI) m/e [M+1]⁺=344.

### Step 4: (1R,3S)-3-(3-((2-(1′-(3-(2,6-dioxopiperidin-3-yl)-2-oxo-2,3-dihydroben-zo[d]oxazol-7-yl)-[1,4′-bipiperidin]-4-yl)-4-fluoro-1,1-dioxido-2,3-dihydrobenzo[d]iso-thiazol-5-yl)amino)-1H-pyrazol-5-yl)cyclopentyl isopropylcarbamate

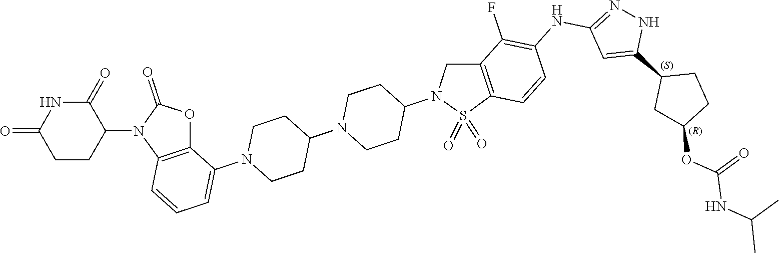

The titled compound was synthesized in the procedures similar to Example 64. ¹H NMR (500 MHz, DMSO) δ 12.00 (s, 1H), 11.20 (s, 1H), 8.76 (s, 1H), 8.28 (s, 1H), 7.51 (d, J=8.6 Hz, 1H), 7.08 (t, J=8.1 Hz, 1H), 6.94 (d, J=7.4 Hz, 1H), 6.77 (d, J=7.9 Hz, 1H), 6.71 (d, J=8.4 Hz, 1H), 5.83 (s, 1H), 5.33 (q, J=12.9, 5.4 Hz, 1H), 5.00 (s, 1H), 4.51 (s, 2H), 3.73 (d, J=11.8 Hz, 2H), 3.63-3.53 (m, 1H), 3.46-3.40 (m, 1H), 3.11-3.04 (m, 1H), 3.00 (d, J=10.8 Hz, 2H), 2.93-2.84 (m, 1H), 2.77 (t, J=11.5 Hz, 2H), 2.69-2.60 (m, 2H), 2.48-2.42 (m, 1H), 2.33 (t, J=10.8 Hz, 2H), 2.18-2.10 (m, 1H), 2.03 (dd, J=15.6, 7.5 Hz, 1H), 1.98-1.79 (m, 7H), 1.71 (dd, J=18.3, 9.9 Hz, 2H), 1.66-1.54 (m, 3H), 1.03 (d, J=6.3 Hz, 6H). LC-MS (ESI): m/z [M+H]⁺=848.57

### Example 126: (1R,3S)-3-(3-((2-(1-((1-(3-(2,6-dioxopiperidin-3-yl)-2-oxo-2,3-dihydrobenzo[d]oxazol-6-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

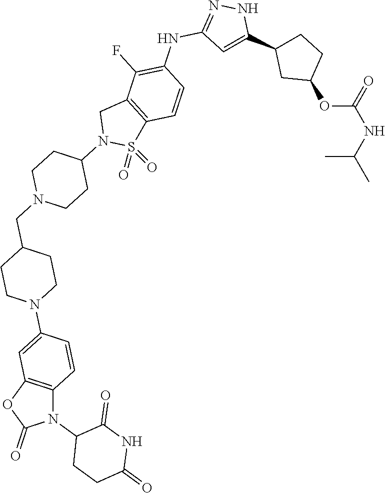 [SMILES: CC(C)NC(=O)O[C@@H]1CC[C@H](c2cc(Nc3ccc4c(c3F)CN(C3CCN(CC5CCN(c6ccc7c(c6)oc(=O)n7C6CCC(=O)NC6=O)CC5)CC3)S4(=O)=O)n[nH]2)C1]

The titled compound was synthesized in the procedures similar to Example 62. ¹H NMR (500 MHz, DMSO-d₆) δ 12.00 (s, 1H), 11.17 (s, 1H), 8.76 (s, 1H), 8.28 (s, 1H), 7.51 (d, J=5 Hz, 1H), 7.12-7.03 (m, 2H), 6.98-6.91 (m, 1H), 6.81-6.75 (m, 1H), 5.83 (s, 1H), 5.33-5.24 (m, 1H), 5.06-4.96 (m, 1H), 4.52 (s, 2H), 3.65-3.52 (m, 3H), 3.48-3.39 (m, 1H), 3.11-3.01 (m, 1H), 2.95-2.82 (m, 3H), 2.74-2.58 (m, 4H), 2.47-2.43 (m, 1H), 2.23-2.08 (m, 3H), 2.07-1.96 (m, 3H), 1.95-1.82 (m, 5H), 1.81-1.55 (m, 6H), 1.28-1.14 (m, 2H), 1.08-0.95 (m, 6H). LC-MS (ESI): m/z [M+H]⁺=862.

### Example 127: (1R,3S)-3-(3-((2-((3S)-1-((1-(3-(2,6-dioxopiperidin-3-yl)-2-oxo-2,3-dihydrobenzo[d]oxazol-6-yl)piperidin-4-yl)methyl)pyrrolidin-3-yl)-4-fluoro-1,1-dioxido-2,3-dihydrobenzo[d]isothiazol-5-yl)amino)-1H-pyrazol-5-yl)cyclopentyl isopropylcarbamate

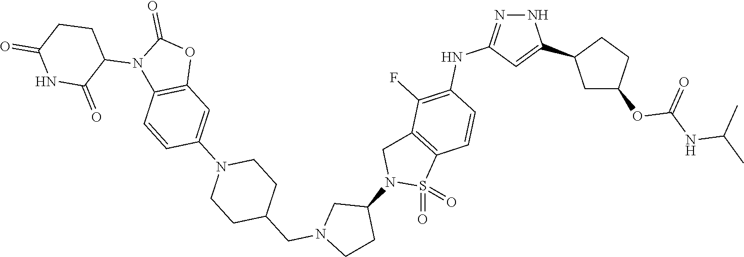 [SMILES: CC(C)NC(=O)O[C@@H]1CC[C@H](c2cc(Nc3ccc4c(c3F)CN([C@H]3CCN(CC5CCN(c6ccc7c(c6)oc(=O)n7C6CCC(=O)NC6=O)CC5)C3)S4(=O)=O)n[nH]2)C1]

### Step1: (1R,3S)-3-(3-((4-fluoro-1,1-dioxido-2-((S)-pyrrolidin-3-yl)-2,3-dihydrobenzo[d]isothiazol-5-yl)amino)-1H-pyrazol-5-yl)cyclopentyl isopropylcarbamate

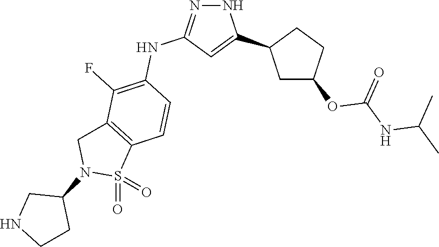 [SMILES: CC(C)NC(=O)O[C@@H]1CC[C@H](c2cc(Nc3ccc4c(c3F)CN([C@H]3CCNC3)S4(=O)=O)n[nH]2)C1]

The titled compound was synthesized in the procedures similar to Example 1. LC-MS (ESI): m/z [M+H]⁺=504.8

### Step 2: (1R,3S)-3-(3-((2-((3S)-1-((1-(3-(2,6-dioxopiperidin-3-yl)-2-oxo-2,3-dihydrobenzo[d]oxazol-6-yl)piperidin-4-yl)methyl)pyrrolidin-3-yl)-4-fluoro-1,1-dioxido-2,3-dihydrobenzo[d]isothiazol-5-yl)amino)-1H-pyrazol-5-yl)cyclopentyl isopropylcarbamate

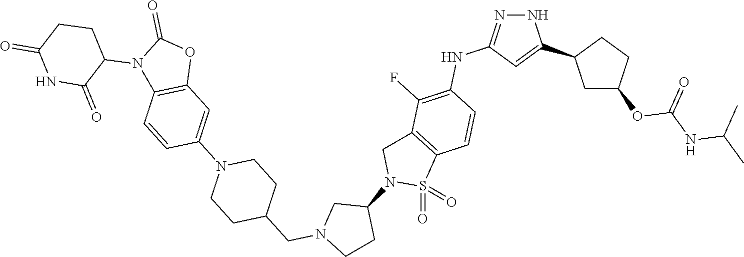 [SMILES: CC(C)NC(=O)O[C@@H]1CC[C@H](c2cc(Nc3ccc4c(c3F)CN([C@H]3CCN(CC5CCN(c6ccc7c(c6)oc(=O)n7C6CCC(=O)NC6=O)CC5)C3)S4(=O)=O)n[nH]2)C1]

The titled compound was synthesized in the procedures similar to Example 62. LC-MS (ESI): m/z [M+H]⁺=848.47; ¹H NMR (500 MHz, DMSO) δ=12.00 (s, 1H), 11.17 (s, 1H), 8.78 (s, 1H), 8.28 (s, 1H), 7.52 (d, J=8.7, 1H), 7.06 (d, J=9.1, 2H), 6.94 (d, J=7.7, 1H), 6.78 (d, J=8.7, 1H), 5.84 (s, 1H), 5.28 (dd, J=12.9, 5.4, 1H), 5.00 (s, 1H), 4.56 (d, J=5.7, 2H), 4.15 (s, 1H), 3.59 (m, 4H), 3.07 (m, 1H), 2.86-2.82 (m, 2H), 2.78 (m, 1H), 2.66-2.62 (m, 4H), 2.59 (m, 1H), 2.46-2.43 (m, 1H), 2.34 (m, 3H), 2.29-2.25 (m, 1H), 2.21-2.10 (m, 3H), 2.03 (m, 2H), 1.91 (m, 1H), 1.81 (m, 2H), 1.72 (m, 2H), 1.59 (m, 2H), 1.29-1.20 (m, 2H), 1.04 (m, 6H).

### Example 128: (1R,3S)-3-(3-((2-(1-((1-(3-(2,6-dioxopiperidin-3-yl)-2-oxo-2,3-dihydrobenzo[d]oxazol-6-yl)azetidin-3-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

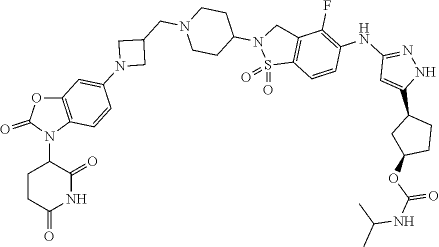 [SMILES: CC(C)NC(=O)O[C@@H]1CC[C@H](c2cc(Nc3ccc4c(c3F)CN(C3CCN(CC5CN(c6ccc7c(c6)oc(=O)n7C6CCC(=O)NC6=O)C5)CC3)S4(=O)=O)n[nH]2)C1]

The titled compound was synthesized in the procedures similar to Example 101. LC-MS (ESI): m/z [M+H]⁺=834.57; ¹H NMR (500 MHz, DMSO) δ=11.99 (s, 1H), 11.16 (s, 1H), 8.76 (s, 1H), 8.29 (s, 1H), 7.51 (d, J=8.6, 1H), 7.04 (d, J=8.5, 1H), 6.93 (s, 1H), 6.53 (s, 1H), 6.23 (d, J=8.3, 1H), 5.83 (s, 1H), 5.27 (dd, J=12.9, 5.3, 1H), 4.99 (s, 1H), 4.51 (s, 2H), 3.90 (t, J=7.4, 2H), 3.57 (m, 1H), 3.42 (m, 1H), 3.06 (m, 1H), 2.89 (m, 4H), 2.63 (m, 4H), 2.57 (m, 2H), 2.14-2.06 (m, 3H), 2.03 (m, 1H), 1.86 (m, 3H), 1.71 (m, 1H), 1.60 (m, 1H), 1.03 (m, 3H).

### Example 129: (1R,3S)-3-(3-((2-(1′-(1-(2,6-dioxopiperidin-3-yl)-4-fluoro-3-isopropyl-2-oxo-2,3-dihydro-1H-benzo[d]imidazol-5-yl)-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

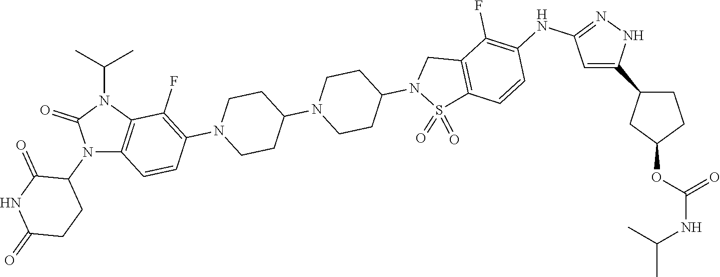 [SMILES: CC(C)NC(=O)O[C@@H]1CC[C@H](c2cc(Nc3ccc4c(c3F)CN(C3CCN(C5CCN(c6ccc7c(c6F)n(C(C)C)c(=O)n7C6CCC(=O)NC6=O)CC5)CC3)S4(=O)=O)n[nH]2)C1]

### Step 1: 1-(2,6-bis(benzyloxy)pyridin-3-yl)-5-bromo-4-fluoro-3-isopropyl-1,3-dihydro-2H-benzo[d]imidazol-2-one

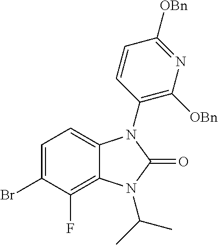 [SMILES: CC(C)n1c(=O)n(-c2ccc(OCc3ccccc3)nc2OCc2ccccc2)c2ccc(Br)c(F)c21]

To a solution of 1-(2,6-bis(benzyloxy)pyridin-3-yl)-5-bromo-4-fluoro-1,3-dihydro-2H-benzo[d]imidazol-2-one (PH-BGUS-H-A554-12) (16 g, 30.7 mmol) in DMF (200 mL) were added 2-iodopropane (10.46 g, 61.5 mmol) and K₂CO₃ (8.36 g, 61.5 mmol) at rt for 2 h under N₂ atmosphere. The resulting solution was extracted with EA. The organic layers were washed with brine. The reaction was concentrated under vacuum. The crude was purified by combi-flash PE:EA=100:0 to 65:35 to give the product (8.9 g, 52%) as light yellow solid. LCMS (M+H) m/z: 563

### Step 2: 1-(2,6-bis(benzyloxy)pyridin-3-yl)-4-fluoro-3-isopropyl-5-(1,4-dioxa-8-azaspiro[4.5]decan-8-yl)-1,3-dihydro-2H-benzo[d]imidazol-2-one

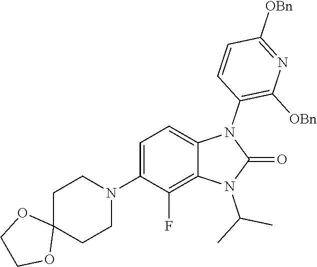 [SMILES: CC(C)n1c(=O)n(-c2ccc(OCc3ccccc3)nc2OCc2ccccc2)c2ccc(N3CCC4(CC3)OCCO4)c(F)c21]

To a solution of 1-(2,6-bis(benzyloxy)pyridin-3-yl)-5-bromo-4-fluoro-3-isopropyl-1,3-dihydro-2H-benzo[d]imidazol-2-one (8.8 g, 15.6 mmol) in Dioxane (100 mL) were added 1,4-dioxa-8-azaspiro[4.5]decane (2.3 mg, 15.6 mmol), Pd-PEPPSI-IpentCl (658 mg, 0.78 mmol) and Cs₂CO₃ (7.6 g, 23.4 mmol) at 100° C. for overnight under N₂ atmosphere. The reaction was concentrated under reduced pressure. The crude was purified by combi-flash PE:EA=100:0 to 65:35 to give the product (7.3 g, 74%) as light yellow solid. LCMS (M+H) m/z: 625.

### Step 3: 3-(4-fluoro-3-isopropyl-2-oxo-5-(1,4-dioxa-8-azaspiro[4.5]decan-8-yl)-2,3-dihydro-1H-benzo[d]imidazol-1-yl)piperidine-2,6-dione

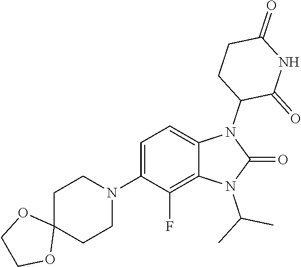 [SMILES: CC(C)n1c(=O)n(C2CCC(=O)NC2=O)c2ccc(N3CCC4(CC3)OCCO4)c(F)c21]

To a solution of 1-(2,6-bis(benzyloxy)pyridin-3-yl)-4-fluoro-3-isopropyl-5-(1,4-dioxa-8-azaspiro[4.5]decan-8-yl)-1,3-dihydro-2H-benzo[d]imidazol-2-one (7.3 g, 11.6 mmol) in THF (80 mL) was added Pd/C (7.3 g) at 50° C. for overnight under H₂ atmosphere. The reaction was filtered. The filtrate was concentrated under vacuum. The residue was without purified to give the product (5 g, 90%) as white solid. LCMS (M+H) m/z: 447.

### Step 4: 3-(4-fluoro-3-isopropyl-2-oxo-5-(4-oxopiperidin-1-yl)-2,3-dihydro-1H-benzo[d]imidazol-1-yl)piperidine-2,6-dione

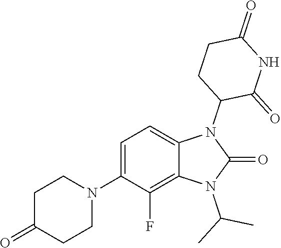 [SMILES: CC(C)n1c(=O)n(C2CCC(=O)NC2=O)c2ccc(N3CCC(=O)CC3)c(F)c21]

To a solution of 3-(4-fluoro-3-isopropyl-2-oxo-5-(1,4-dioxa-8-azaspiro[4.5]decan-8-yl)-2,3-dihydro-1H-benzo[d]imidazol-1-yl)piperidine-2,6-dione (5 g, 11.2 mmol) in H₂SO₄ (10 mL) at 0° C. for 2 h. The reaction was concentrated under vacuum. The filtrate cake was concentrated under vacuum to give the product (3.5 g, 70%) as light yellow solid. LCMS (M+H) m/z: 403

### Step 5:(1R,3S)-3-(3-((2-(1′-(1-(2,6-dioxopiperidin-3-yl)-4-fluoro-3-isopropyl-2-oxo-2,3-dihydro-1H-benzo[d]imidazol-5-yl)-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

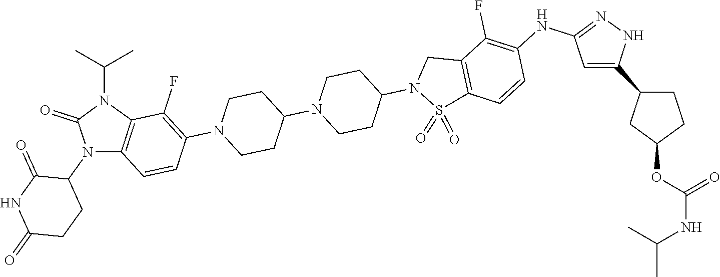 [SMILES: CC(C)NC(=O)O[C@@H]1CC[C@H](c2cc(Nc3ccc4c(c3F)CN(C3CCN(C5CCN(c6ccc7c(c6F)n(C(C)C)c(=O)n7C6CCC(=O)NC6=O)CC5)CC3)S4(=O)=O)n[nH]2)C1]

The titled compound was synthesized in the procedures similar to Example 59. ¹H NMR (500 MHz, DMSO) δ 11.98 (s, 1H), 11.07 (s, 1H), 8.74 (s, 1H), 8.27-8.21 (m, 1H), 7.50 (d, J=8.6 Hz, 1H), 6.89-6.87 (m, 1H), 6.81-6.79 (m, 1H), 6.76-6.61 (m, 1H), 5.85-5.81 (m, 1H), 5.13-5.08 (m, 1H), 5.02-4.85 (m, 1H), 4.78-4.65 (m, 1H), 4.50 (s, 2H), 3.57-3.43 (m, 2H), 3.17-2.93 (m, 3H), 2.91-2.75 (m, 1H), 2.66-2.38 (m, 5H), 2.37-2.23 (m, 1H), 2.05-1.96 (m, 2H), 1.94-1.85 (m, 4H), 1.84-1.77 (m, 3H), 1.75-1.50 (m, 6H), 1.41 (d, J=6.7 Hz, 6H), 1.00 (d, J=5.9 Hz, 6H). LC-MS (ESI): m/z [M+H]⁺=907.4;

### Example 130: (1R,3S)-3-(3-((2-((4R)-1′-(1-(2,6-dioxopiperidin-3-yl)-4-fluoro-3-methyl-2-oxo-2,3-dihydro-1H-benzo[d]imidazol-5-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

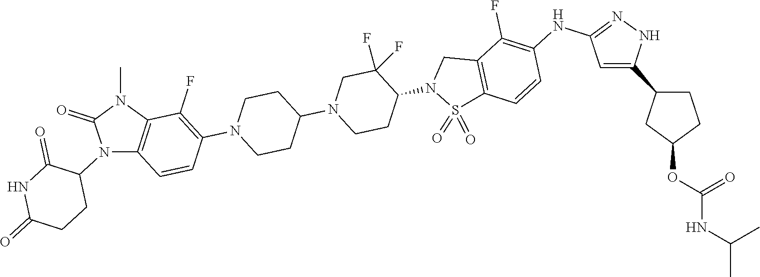 [SMILES: CC(C)NC(=O)O[C@@H]1CC[C@H](c2cc(Nc3ccc4c(c3F)CN([C@@H]3CCN(C5CCN(c6ccc7c(c6F)n(C)c(=O)n7C6CCC(=O)NC6=O)CC5)CC3(F)F)S4(=O)=O)n[nH]2)C1]

The titled compound was synthesized in the procedures similar to Example 45. ¹H NMR (500 MHz, DMSO) δ 12.00 (s, 1H), 11.10 (s, 1H), 8.82 (s, 1H), 8.33 (t, J=8.0 Hz, 1H), 7.58 (d, J=8.6 Hz, 1H), 6.85 (d, J=8.5 Hz, 1H), 6.81-6.68 (m, 2H), 5.84 (s, 1H), 5.33 (dd, J=12.6, 5.1 Hz, 1H), 5.06-4.96 (m, 1H), 4.75 (d, J=14.2 Hz, 1H), 4.54 (d, J=13.1 Hz, 1H), 4.05-3.90 (m, 1H), 3.47 (s, 3H), 3.25-3.16 (m, 1H), 3.13-2.99 (m, 2H), 2.97-2.61 (m, 10H), 2.45-2.42 (m, 1H), 2.20-2.19 (m, 1H), 2.04-1.96 (m, 2H), 1.94-1.78 (m, 4H), 1.75-1.55 (m, 5H), 1.03 (d, J=6.3 Hz, 6H). LC-MS (ESI): m/z [M+H]⁺=915.4;

### Example 131a and 131b and 131c and 131d: (1R,3S)-3-(3-((2-((S)-1′-(1-((S)-2,6-dioxopiperidin-3-yl)-7-fluoro-3-methyl-2-oxo-2,3-dihydro-1H-benzo[d]imidazol-5-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate and (1R,3S)-3-(3-((2-((S)-1′-(1-((R)-2,6-dioxopiperidin-3-yl)-7-fluoro-3-methyl-2-oxo-2,3-dihydro-1H-benzo[d]imidazol-5-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate and (1R,3S)-3-(3-((2-((R)-1′-(1-((R)-2,6-dioxopiperidin-3-yl)-7-fluoro-3-methyl-2-oxo-2,3-dihydro-1H-benzo[d]imidazol-5-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate and (1R,3S)-3-(3-((2-((R)-1′-(1-((S)-2,6-dioxopiperidin-3-yl)-7-fluoro-3-methyl-2-oxo-2,3-dihydro-1H-benzo[d]imidazol-5-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

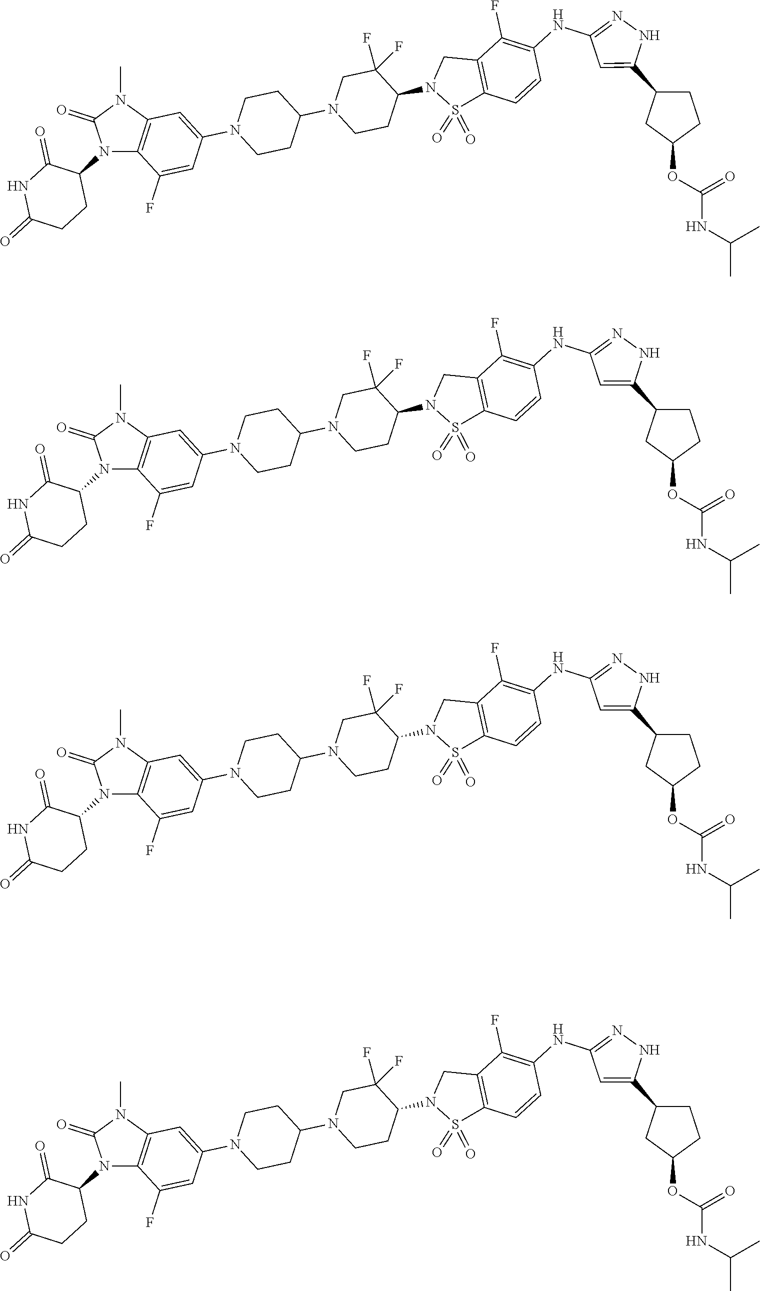

The titled compound was synthesized in the procedures similar to Example 60 in a racemic form, which was further separated by Chiral Prep-HPLC to give:

Enantiomer 1 (Example 131a, 100% ee); Retention time: 9.731 min. ¹H NMR (500 MHz, DMSO) δ 12.01 (s, 1H), 11.08 (s, 1H), 8.82 (s, 1H), 8.33 (t, J=8.0 Hz, 1H), 7.58 (d, J=8.7 Hz, 1H), 6.94 (d, J=7.5 Hz, 1H), 6.70 (s, 1H), 6.59-6.48 (m, 1H), 5.87 (d, J=1.8 Hz, 1H), 5.45-5.33 (m, 1H), 5.06-4.96 (m, 1H), 4.80-4.66 (m, 1H), 4.61-4.45 (m, 1H), 4.07-3.88 (m, 1H), 3.79-3.65 (m, 2H), 3.58-3.54 (m, 1H), 3.32 (s, 3H), 3.26-3.19 (m, 1H), 3.13-2.97 (m, 3H), 2.82-2.59 (m, 4H), 2.59-2.51 (m, 1H), 2.49-2.44 (m, 1H), 2.23-1.97 (m, 4H), 1.92-1.88 (m, 1H), 1.85-1.79 (m, 3H), 1.78-1.67 (m, 2H), 1.67-1.51 (m, 3H), 1.03 (d, J=6.3 Hz, 6H). LC-MS (ESI): m/z [M+H]⁺=915.3;

Enantiomer 2 (Example 131b, 100% ee); Retention time: 12.237 min. ¹H NMR (500 MHz, DMSO) δ 12.01 (s, 1H), 11.08 (s, 1H), 8.82 (s, 1H), 8.33 (t, J=8.0 Hz, 1H), 7.58 (d, J=8.7 Hz, 1H), 6.94 (d, J=7.5 Hz, 1H), 6.70 (s, 1H), 6.59-6.48 (m, 1H), 5.87 (d, J=1.8 Hz, 1H), 5.45-5.33 (m, 1H), 5.06-4.96 (m, 1H), 4.80-4.66 (m, 1H), 4.61-4.45 (m, 1H), 4.07-3.88 (m, 1H), 3.79-3.65 (m, 2H), 3.58-3.54 (m, 1H), 3.32 (s, 3H), 3.26-3.19 (m, 1H), 3.13-2.97 (m, 3H), 2.82-2.59 (m, 4H), 2.59-2.51 (m, 1H), 2.49-2.44 (m, 1H), 2.23-1.97 (m, 4H), 1.92-1.88 (m, 1H), 1.85-1.79 (m, 3H), 1.78-1.67 (m, 2H), 1.67-1.51 (m, 3H), 1.03 (d, J=6.3 Hz, 6H). LC-MS (ESI): m/z [M+H]⁺=915.3;

Enantiomer 3 (Example 131c, 100% ee); Retention time: 13.827 min. ¹H NMR (500 MHz, DMSO) δ 12.01 (s, 1H), 11.08 (s, 1H), 8.82 (s, 1H), 8.33 (t, J=8.0 Hz, 1H), 7.58 (d, J=8.7 Hz, 1H), 6.94 (d, J=7.5 Hz, 1H), 6.70 (s, 1H), 6.59-6.48 (m, 1H), 5.87 (d, J=1.8 Hz, 1H), 5.45-5.33 (m, 1H), 5.06-4.96 (m, 1H), 4.80-4.66 (m, 1H), 4.61-4.45 (m, 1H), 4.07-3.88 (m, 1H), 3.79-3.65 (m, 2H), 3.58-3.54 (m, 1H), 3.32 (s, 3H), 3.26-3.19 (m, 1H), 3.13-2.97 (m, 3H), 2.82-2.59 (m, 4H), 2.59-2.51 (m, 1H), 2.49-2.44 (m, 1H), 2.23-1.97 (m, 4H), 1.92-1.88 (m, 1H), 1.85-1.79 (m, 3H), 1.78-1.67 (m, 2H), 1.67-1.51 (m, 3H), 1.03 (d, J=6.3 Hz, 6H). LC-MS (ESI): m/z [M+H]⁺=915.3; Enantiomer 4 (Example 131d, 100% ee); Retention time: 16.544 min. ¹H NMR (500 MHz, DMSO) δ 12.01 (s, 1H), 11.08 (s, 1H), 8.82 (s, 1H), 8.33 (t, J=8.0 Hz, 1H), 7.58 (d, J=8.7 Hz, 1H), 6.94 (d, J=7.5 Hz, 1H), 6.70 (s, 1H), 6.59-6.48 (m, 1H), 5.87 (d, J=1.8 Hz, 1H), 5.45-5.33 (m, 1H), 5.06-4.96 (m, 1H), 4.80-4.66 (m, 1H), 4.61-4.45 (m, 1H), 4.07-3.88 (m, 1H), 3.79-3.65 (m, 2H), 3.58-3.54 (m, 1H), 3.32 (s, 3H), 3.26-3.19 (m, 1H), 3.13-2.97 (m, 3H), 2.82-2.59 (m, 4H), 2.59-2.51 (m, 1H), 2.49-2.44 (m, 1H), 2.23-1.97 (m, 4H), 1.92-1.88 (m, 1H), 1.85-1.79 (m, 3H), 1.78-1.67 (m, 2H), 1.67-1.51 (m, 3H), 1.03 (d, J=6.3 Hz, 6H). LC-MS (ESI): m/z [M+H]⁺=915.3; Chiral analytical method: Column: ICHIRALPAK IC-5 4.6*150 mm 5 μm; Mobile phase: A for MtBE (0.1%2M NH3 MeOH) (%) and B for MeOH:DCM=50:50(%); Gradient: Mobile Phase A: Mobile Phase B=40:60 (v/v); HPLC Equipment: HPLC-Agilent, Back pressure: 100 bar; Column temperature: 35° C.

Chiral Prep-HPLC Condition: CHIRALPAK IC-5 250*20.0 mml, 5 μm; Mobile phase: A for MtBE (0.1%2M NH3 MeOH) (%) and B for MeOH:DCM=50:50; Gradient: Mobile Phase A: Mobile Phase B=40:60 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 132: (1R,3S)-3-(3-((2-((4R)-1-(2-(1-(2,6-dioxopiperidin-3-yl)-4-fluoro-3-methyl-2-oxo-2,3-dihydro-1H-benzo[d]imidazol-5-yl)ethyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl isopropylcarbamate

### Step 1: 3-(5-bromo-4-fluoro-3-methyl-2-oxo-2,3-dihydro-1H-benzo[d]imidazol-1-yl)-1-(4-methoxybenzyl)piperidine-2,6-dione

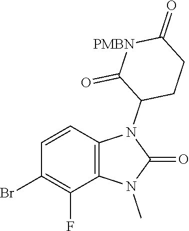

To a solution of 6-bromo-7-fluoro-1-methyl-1,3-dihydro-2H-benzo[d]imidazol-2-one (1 g, 4 mmol) in THF (15 mL) was added t-BuOK (548 mg, 4.8 mmol) at 0° C. for 30 min, was added 1-(4-methoxybenzyl)-2,6-dioxopiperidin-3-yl trifluoromethanesulfonate (1.8 g, 4.8 mmol) in THF (10 mL) at 0° C. for overnight under N₂ atmosphere. The resulting solution was extracted with EA. The organic layers were washed with brine. The reaction was concentrated under reduced pressure. The crude was purified by combi-flash PE:EA=100:0 to 65:35 to give the product (1 g, 52%) as light yellow solid. LCMS (M+H) m/z: 476.

### Step 2: 3-(5-bromo-4-fluoro-3-methyl-2-oxo-2,3-dihydro-1H-benzo[d]imidazol-1-yl)piperidine-2,6-dione

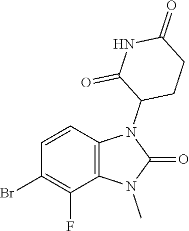 [SMILES: Cn1c(=O)n(C2CCC(=O)NC2=O)c2ccc(Br)c(F)c21]

To a solution of 3-(5-bromo-4-fluoro-3-methyl-2-oxo-2,3-dihydro-1H-benzo[d]imidazol-1-yl)-1-(4-methoxybenzyl)piperidine-2,6-dione (1 g, 2 mmol) in TFA (10 mL) was added TfOH (3.1 g, 20 mmol) at 65° C. for 2 h under N₂ atmosphere. The reaction was concentrated under reduced pressure. The crude was purified by combine-flash PE:EA=100:0 to 65:35 to give the product (380 mg, 50%) as grey solid. LCMS (M+H) m/z: 356.

### Step 3: (E)-3-(5-(2-ethoxyvinyl)-4-fluoro-3-methyl-2-oxo-2,3-dihydro-1H-benzo[d]imidazol-1-yl)piperidine-2,6-dione

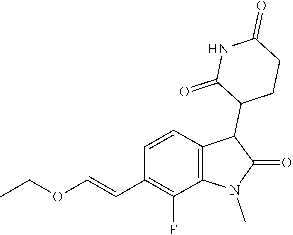 [SMILES: CCO/C=C/c1ccc2c(c1F)N(C)C(=O)C2C1CCC(=O)NC1=O]

To a solution of 3-(5-bromo-4-fluoro-3-methyl-2-oxo-2,3-dihydro-1H-benzo[d]imidazol-1-yl)piperidine-2,6-dione (260 mg, 0.73 mmol) in DMF (8 mL) and H₂O (2 mL) were added (E)-2-(2-ethoxyvinyl)-4,4,5,5-tetramethyl-1,3,2-dioxaborolane (173 mg, 0.87 mmol), Pd(dtbpf)Cl₂ (47 mg, 0.07 mmol) and CsF (222 mg, 1.4 mmol) at 80° C. for overnight under N₂ atmosphere. The reaction was extracted with EA. The organic layers were washed with brine. dried over anhydrous Na₂SO₄ The reaction was concentrated under reduced pressure. The crude was purified by Prep-TLC to give the product (210 mg, 84%) as grey solid. LCMS (M+H) m/z: 348.

### Step 4: 2-(1-(2,6-dioxopiperidin-3-yl)-4-fluoro-3-methyl-2-oxo-2,3-dihydro-1H-benzo[d]imidazol-5-yl)acetaldehyde

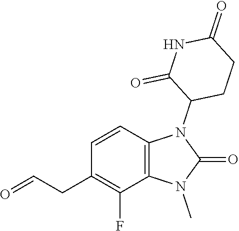 [SMILES: Cn1c(=O)n(C2CCC(=O)NC2=O)c2ccc(CC=O)c(F)c21]

To a flask was added (E)-3-(5-(2-ethoxyvinyl)-4-fluoro-3-methyl-2-oxo-2,3-dihydro-1H-benzo[d]imidazol-1-yl)piperidine-2,6-dione (210 mg, 0.6 mmol) and 6 mL formic acid. The mixture was stirred at room temperature for 2 h. The solution was concentrated under reduced pressure to afford the product. LC-MS (ESI): m/z [M+H]⁺=320.3.

### Step 5: (1R,3S)-3-(3-((2-((4R)-1-(2-(1-(2,6-dioxopiperidin-3-yl)-4-fluoro-3-methyl-2-oxo-2,3-dihydro-1H-benzo[d]imidazol-5-yl)ethyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl isopropylcarbamate

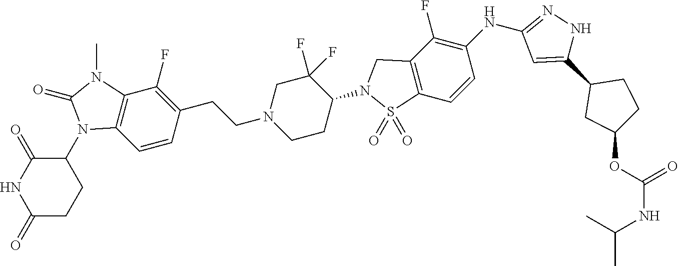 [SMILES: CC(C)NC(=O)O[C@@H]1CC[C@H](c2cc(Nc3ccc4c(c3F)CN([C@@H]3CCN(CCc5ccc6c(c5F)n(C)c(=O)n6C5CCC(=O)NC5=O)CC3(F)F)S4(=O)=O)n[nH]2)C1]

The titled compound was synthesized in the procedures similar to Example 37. LC-MS (ESI): m/z [M+H]⁺=860.71; ¹H NMR (500 MHz, DMSO) δ=12.01 (s, 1H), 11.11 (s, 1H), 8.82 (s, 1H), 8.33 (t, J=8.1, 1H), 7.58 (d, J=8.6, 1H), 7.02-6.97 (m, 1H), 6.93 (m, 2H), 5.84 (s, 1H), 5.37 (dd, J=12.8, 5.2, 1H), 5.00 (s, 1H), 4.74 (d, J=14.1, 1H), 4.53 (d, J=13.3, 1H), 3.98 (m, 1H), 3.56 (m, 2H), 3.49 (m, 3H), 3.06 (m, 2H), 2.88 (m, 1H), 2.81 (m, 2H), 2.70 (m, 2H), 2.66 (m, 2H), 2.42-2.40 (m, 1H), 2.18 (m, 1H), 2.03 (m, 2H), 1.91 (m, 1H), 1.83 (m, 1H), 1.71 (m, 2H), 1.60 (m, 1H), 1.03 (m, 6H).

### Example 133: (1R,3S)-3-(3-((2-((4R)-1-((1-(1-(2,6-dioxopiperidin-3-yl)-3-methyl-2-oxo-2,3-dihydro-1H-benzo[d]imidazol-5-yl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropylcarbamate

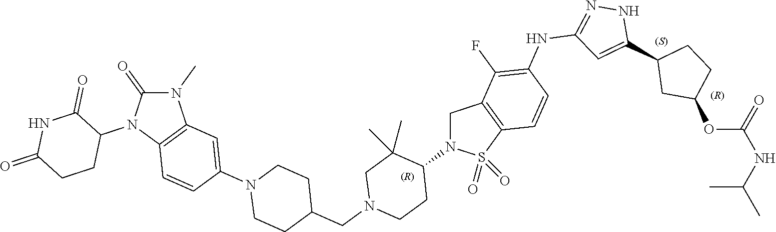

### Step 1: (1R,3S)-3-(3-((2-((4R)-1-((1-(1-(2,6-dioxopiperidin-3-yl)-3-methyl-2-oxo-2,3-dihydro-1H-benzo[d]imidazol-5-yl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropylcarbamate

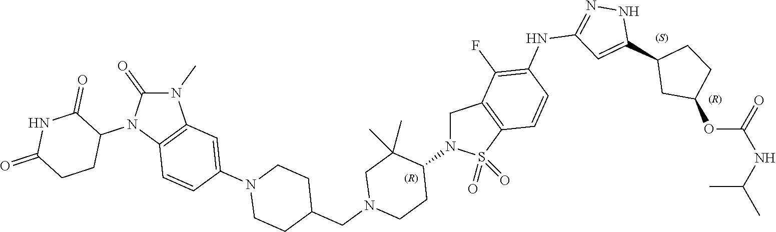

To a solution of (1R,3S)-3-(3-((2-((R)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropyl carbamate (50 mg, 0.091 mmol) and 1-(1-(2,6-dioxopiperidin-3-yl)-3-methyl-2-oxo-2,3-dihydro-1H-benzo[d]imidazol-5-yl)piperidine-4-carbaldehyde (51 mg, 0.137 mmol) in DMA (2 mL) was stirred for 30 min, then STAB (39 mg, 0.182 mmol) was added. the reaction mixture was stirred for 16 hours at room temperature. The resulting mixture was concentrated under reduce pressure, the residue was purified by column chromatography on silica gel, eluting with DCM/MeOH (10:1) to afford the product (40 mg, 49%). The residue 40 mg was purified by prep HPLC (Waters XSelect C18: RD-CO-094 column, eluting with a gradient of acetonitrile/water containing 0.1% FA, 20%-40%) to afford the product (25 mg, 30%). LC-MS (ESI): m/z [M+H]⁺=903.50.

¹H NMR (500 MHz, DMSO) δ 11.99 (s, 1H), 11.06 (s, 1H), 8.77 (s, 1H), 8.29 (s, 1H), 7.54 (d, J=8.6 Hz, 1H), 6.93 (t, J=8.4 Hz, 2H), 6.83 (s, 1H), 6.64 (d, J=7.1 Hz, 1H), 5.83 (s, 1H), 5.28 (dd, J=12.8, 5.4 Hz, 1H), 5.00 (s, 1H), 4.55 (s, 2H), 3.65-3.55 (m, 3H), 3.30 (s, 3H), 3.23 (d, J=10.8 Hz, 1H), 3.12-3.02 (m, 1H), 3.00-2.85 (m, 2H), 2.70-2.57 (m, 6H), 2.18-1.49 (m, 15H), 1.25 (d, J=7.6 Hz, 2H), 1.13 (s, 3H), 1.06-1.00 (m, 6H), 0.94 (s, 3H).

### Example 134: (1R,3S)-3-(3-((2-((S)-1-((1-(1-((S)-2,6-dioxopiperidin-3-yl)-7-fluoro-3-methyl-2-oxo-2,3-dihydro-1H-benzo[d]imidazol-5-yl)piperidin-4-yl)methyl)-3,3-dimethyl-piperidin-4-yl)-4-fluoro-1,1-dioxido-2,3-dihydrobenzo[d]isothiazol-5-yl)amino)-1H-pyrazol-5-yl)cyclopentyl isopropylcarbamate

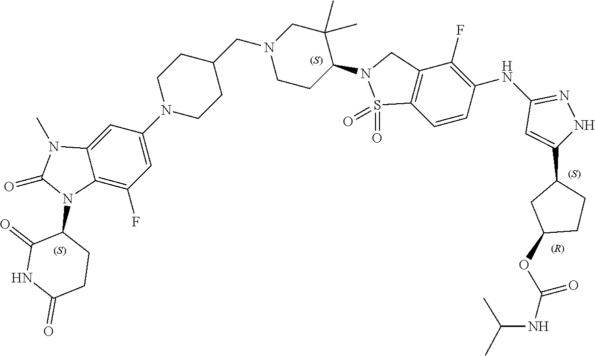

### Step 1: tert-butyl (S)-4-(4-fluoro-5-((5-((1S,3R)-3-((isopropylcarbamoyl)oxy)cyclo-pentyl)-1H-pyrazol-3-yl)amino)-1,1-dioxidobenzo[d]isothiazol-2(3H)-yl)-3,3-dimethylpi-peridine-1-carboxylate

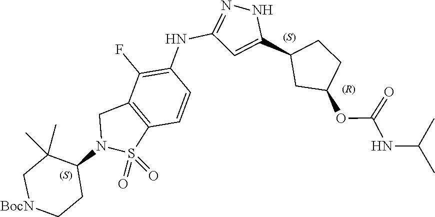

To a mixture of (1R,3S)-3-(3-amino-1H-pyrazol-5-yl)cyclopentyl isopropylcarbamate (1 g, 3.97 mmol) and tert-butyl (S)-4-(5-bromo-4-fluoro-1,1-dioxidobenzo[d]isothiazol-2(3H)-yl)-3,3-dimethylpiperidine-1-carboxylate (1.89 g, 3.97 mmol) in t-BuOH (30 mL) was added Brettphos Pd G3 (362 mg, 0.40 mmol) and K₂CO₃ (1.64 g, 11.88 mmol). The reaction mixture was stirred under nitrogen atmosphere at 110° C. for 16 h. The mixture was filtered, and the filtrate was concentrated under reduced pressure. The residue was purified by silica gel column chromatography, eluting with PE/EA (1:9) to afford the product (2.2 g, 86% yield). LC-MS (ESI): m/z [M+H]⁺=649.3.

### Step 2: (1R,3S)-3-(3-((2-((S)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropylcarbamate

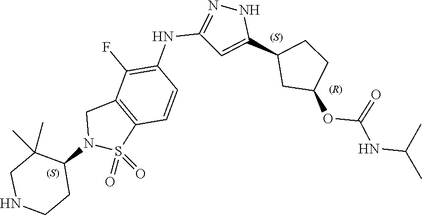

To a solution of tert-butyl (S)-4-(4-fluoro-5-((5-((1S,3R)-3-((isopropylcarbamoyl)o-xy)cyclo-pentyl)-1H-pyrazol-3-yl)amino)-1,1-dioxidobenzo[d]isothiazol-2(3H)-yl)-3,3-dimethylpi-peridine-1-carboxylate (2.2 g, 3.40 mmol) in dichloromethane (20 mL) was added trifluoroacetic acid (10 mL). The reaction mixture was stirred for 1 hours at room temperature. The resulting mixture was concentrated, dried over in vacuum, to afford the product (1.86 g, 100% yield). LC-MS (ESI): m/z [M+H]⁺=548.3.

### Step 3: (1R,3S)-3-(3-((2-((S)-1-((1-(1-((S)-2,6-dioxopiperidin-3-yl)-7-fluoro-3-methyl-2-oxo-2,3-dihydro-1H-benzo[d]imidazol-5-yl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropylcarbamate

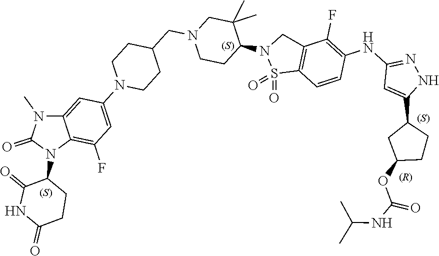

To a solution of (1R,3S)-3-(3-((2-((S)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropyl carbamate (50 mg, 0.091 mmol) and 1-(4-(2,4-dioxotetrahydropyrimidin-1(2H)-yl)phenyl)-pip-eridine-4-carbaldehyde (50 mg, 0.137 mmol) in DMA (3 mL) was stirred for 30 min, then STAB (39 mg, 0.182 mmol) was added. the reaction mixture was stirred for 16 hours at room temperature. The resulting mixture was concentrated under reduce pressure, the residue was purified by column chromatography on silica gel, eluting with DCM/MeOH (10:1) to afford the product (60 mg). The residue 40 mg was purified by prep HPLC (Waters XSelect C18: RD-CO-094 column, eluting with a gradient of acetonitrile/water containing 0.1% FA, 20%-40%) to afford the product (40 mg, 49%). LC-MS (ESI): m/z [M+H]⁺=921.68.

Example 134 (100% ee); Retention time: 5.802 min. ¹H NMR (500 MHz, DMSO) δ 11.99 (s, 1H), 11.07 (s, 1H), 8.77 (s, 1H), 8.31 (t, J=7.9 Hz, 1H), 7.54 (d, J=8.6 Hz, 1H), 6.94 (d, J=7.5 Hz, 1H), 6.68 (s, 1H), 6.52 (d, J=15.0 Hz, 1H), 5.83 (s, 1H), 5.41 (s, 1H), 5.00 (s, 1H), 4.54 (s, 2H), 3.67 (s, 2H), 3.58 (dd, J=13.3, 6.5 Hz, 1H), 3.33 (s, 3H), 3.22 (dd, J=12.2, 3.7 Hz, 1H), 3.14-2.89 (m, 3H), 2.74-2.53 (m, 7H), 2.32-1.89 (m, 8H), 1.86-1.56 (m, 7H), 1.51 (d, J=9.6 Hz, 1H), 1.26-1.18 (m, 2H), 1.13 (s, 3H), 1.06-1.00 (m, 6H), 0.94 (s, 3H).

Chiral analytical method: Column: i-Cellulose-5 4.6*250 mm 5 μm; Mobile phase: A for MtBE (0.1%2M NH3 MeOH)(%) and B for MeOH:DCM=50:50 (v/v); Gradient: Mobile Phase A: Mobile Phase B=30:70 (v/v); HPLC Equipment: Agilent 1260 Infinity II (ARD_LC-808); Column temperature: 25° C.

Chiral Prep-HPLC Condition: i-Cellulose-5 4.6*250 mm 5 μm; Mobile phase: A for MtBE (0.1%2M NH3 MeOH)(%) and B for MeOH:DCM=50:50; Gradient: Mobile Phase A: Mobile Phase B=30:70 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 135: (1R,3S)-3-(3-((2-((R)-1-((1-(1-((S)-2,6-dioxopiperidin-3-yl)-7-fluoro-3-methyl-2-oxo-2,3-dihydro-1H-benzo[d]imidazol-5-yl)piperidin-4-yl)methyl)-3,3-dimethylp-iperidin-4-yl)-4-fluoro-1,1-dioxido-2,3-dihydrobenzo[d]isothiazol-5-yl)amino)-1H-pyrazol-5-yl)cyclopentyl isopropylcarbamate

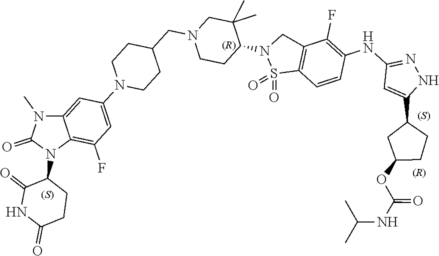

The titled compound was synthesized in the procedures similar to Example 134 in a racemic form, which was further separated by Chiral Prep-HPLC to give the product.

Example 135 (100% ee); Retention time: 6.201 min. ¹H NMR (500 MHz, DMSO) δ 11.98 (s, 1H), 11.07 (s, 1H), 8.76 (s, 1H), 8.31 (t, J=7.9 Hz, 1H), 7.54 (d, J=8.6 Hz, 1H), 6.94 (d, J=7.4 Hz, 1H), 6.68 (s, 1H), 6.52 (d, J=14.7 Hz, 1H), 5.83 (s, 1H), 5.40 (s, 1H), 5.00 (s, 1H), 4.54 (s, 2H), 3.73-3.53 (m, 3H), 3.33 (s, 3H), 3.26-3.19 (m, 1H), 3.10-2.92 (m, 3H), 2.74-2.57 (m, 5H), 2.28-1.89 (m, 8H), 1.86-1.57 (m, 7H), 1.51 (d, J=10.4 Hz, 1H), 1.27-1.18 (m, 2H), 1.13 (s, 3H), 1.06-1.00 (m, 6H), 0.94 (s, 3H). LC-MS (ESI): m/z [M+H]⁺=921.68.

Chiral analytical method: Column: i-Cellulose-5 4.6*250 mm 5 μm; Mobile phase: A for MtBE (0.1%2M NH3 MeOH)(%) and B for MeOH:DCM=50:50 (v/v); Gradient: Mobile Phase A: Mobile Phase B=30:70 (v/v); HPLC Equipment: Agilent 1260 Infinity II (ARD_LC-808); Column temperature: 25° C.

Chiral Prep-HPLC Condition: i-Cellulose-5 4.6*250 mm 5 μm; Mobile phase: A for MtBE (0.1%2M NH3 MeOH)(%) and B for MeOH:DCM=50:50; Gradient: Mobile Phase A: Mobile Phase B=30:70 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 136: (1R,3S)-3-(3-((2-(1-((1-(1-((R)-2,6-dioxopiperidin-3-yl)-4,7-difluoro-3-methyl-2-oxo-2,3-dihydro-1H-benzo[d]imidazol-5-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

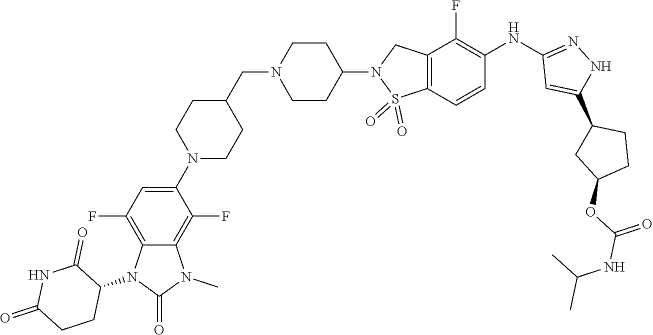 [SMILES: CC(C)NC(=O)O[C@@H]1CC[C@H](c2cc(Nc3ccc4c(c3F)CN(C3CCN(CC5CCN(c6cc(F)c7c(c6F)n(C)c(=O)n7[C@@H]6CCC(=O)NC6=O)CC5)CC3)S4(=O)=O)n[nH]2)C1]

The titled compound was synthesized in the procedures similar to Example 104 in a racemic form, which was further separated by Chiral Prep-HPLC to give Example 136 (100% ee); Retention time: 9.74 min. ¹H NMR (500 MHz, DMSO) δ 11.99 (s, 1H), 11.11 (s, 1H), 8.76 (s, 1H), 8.30 (s, 1H), 7.51 (d, J=8.6 Hz, 1H), 6.94 (d, J=7.7 Hz, 1H), 6.66 (d, J=7.8 Hz, 1H), 5.83 (s, 1H), 5.45 (s, 1H), 5.00 (s, 1H), 4.52 (s, 2H), 3.58-3.57 (m, 1H), 3.50 (s, 3H), 3.44 (s, 2H), 3.25 (s, 2H), 3.12-3.01 (m, 1H), 2.90 (s, 2H), 2.64 (s, 4H), 2.36 (s, 1H), 2.20 (s, 2H), 2.10-2.02 (m, 3H), 1.97-1.84 (m, 5H), 1.82-1.79 (m, 2H), 1.71 (s, 2H), 1.62 (s, 2H), 1.33-1.19 (m, 3H), 1.03-1.02 (m, 6H). LC-MS (ESI): m/z [M+H]⁺=911.48.

Chiral analytical method: Column: I-Cellulose-5, 4.6 mm×250 mm, 5 um; Mobile phase: A for Hexane and B for EtOH (0.1% 2M NH₃ MeOH); Gradient: Mobile Phase A: Mobile Phase B=30:70 (v/v); HPLC Equipment: HPLC-Agilent, Back pressure: 100 bar; Column temperature: 35° C.

Chiral Prep-HPLC Condition: Column: I-Cellulose-5, 21.2 mm×250 mm, 5 um; Mobile phase: A for Hexane and B for EtOH (0.2% 2M NH₃ MeOH); Gradient: Mobile Phase A: Mobile Phase B=30:70 (v/v); Flow Rate: 20 mL/min, Wave Length: UV 200 nm and 270 nm, Prep-HPLC Equipment: Prep-HPLC-Gilson, Back pressure: 100 bar; Column temperature: 25° C.

### Example 137: (1R,3S)-3-(3-((2-((4R)-1′-(1-(2,6-dioxopiperidin-3-yl)-3,4-dimethyl-2-oxo-2,3-dihydro-1H-benzo[d]imidazol-5-yl)-3,3-dimethyl-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

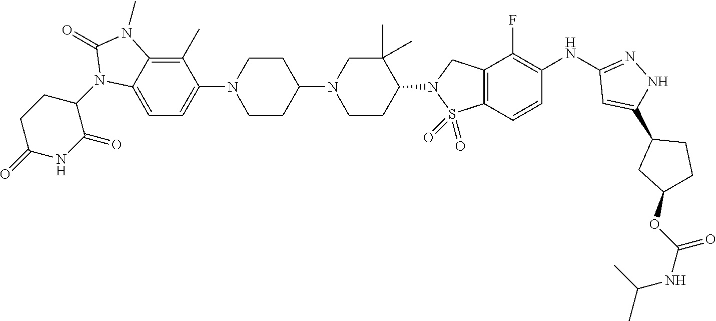 [SMILES: Cc1c(N2CCC(N3CC[C@@H](N4Cc5c(ccc(Nc6cc([C@H]7CC[C@@H](OC(=O)NC(C)C)C7)[nH]n6)c5F)S4(=O)=O)C(C)(C)C3)CC2)ccc2c1n(C)c(=O)n2C1CCC(=O)NC1=O]

The titled compound was synthesized in the procedures similar to Example 43. ¹H NMR (500 MHz, DMSO) δ 11.99 (s, 1H), 11.06 (s, 1H), 8.77 (s, 1H), 8.30 (s, 1H), 7.54 (d, J=8.6 Hz, 1H), 6.94 (d, J=7.1 Hz, 1H), 6.86 (d, J=8.3 Hz, 1H), 6.78 (d, J=8.4 Hz, 1H), 5.84 (s, 1H), 5.32-5.29 (m, 1H), 5.00 (s, 1H), 4.54 (s, 2H), 3.58 (s, 3H), 3.23-3.20 (s, 1H), 3.13-3.01 (m, 2H), 2.99-2.96 (m, 2H), 2.91-2.85 (m, 1H), 2.71-2.66 (dm, 1H), 2.63-2.60 (m, 4H), 2.54 (s, 4H), 2.47-2.43 (m, 1H), 2.36-2.28 (m, 2H), 2.18-1.88 (m, 5H), 1.85-1.50 (m, 8H), 1.11 (s, 3H), 1.04-1.02 (m, 6H), 0.96 (s, 3H). LC-MS (ESI): m/z [M+H]⁺=903.56.

### Example 138: (1R,3S)-3-(3-((2-((4S)-1-((1-(1-(2,6-dioxopiperidin-3-yl)-3-methyl-2-oxo-2,3-dihydro-1H-benzo[d]imidazol-5-yl)piperidin-4-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl isopropylcarbamate

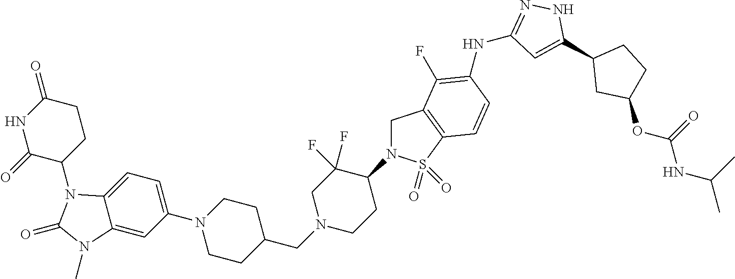 [SMILES: CC(C)NC(=O)O[C@@H]1CC[C@H](c2cc(Nc3ccc4c(c3F)CN([C@H]3CCN(CC5CCN(c6ccc7c(c6)n(C)c(=O)n7C6CCC(=O)NC6=O)CC5)CC3(F)F)S4(=O)=O)n[nH]2)C1]

The titled compound was synthesized in the procedures similar to Example 36. ¹H NMR (500 MHz, DMSO) δ 11.98 (s, 1H), 10.20 (s, 1H), 8.75 (s, 1H), 8.35-8.30 (m, 1H), 7.57-7.48 (m, 1H), 7.11-7.02 (m, 2H), 6.74 (s, 1H), 6.52-6.37 (m, 2H), 5.86-5.82 (m, 1H), 4.97-4.85 (s, 1H), 4.50-4.47 (s, 2H), 3.80-3.66 (m, 4H), 3.67-3.53 (m, 2H), 3.58 (s, 3H), 3.05-3.00 (m, 1H), 2.80-2.74 (m, 2H), 2.69-2.63 (m, 3H), 2.35-2.10 (m, 3H), 2.04-1.94 (m, 3H), 1.94-1.77 (m, 6H), 1.75-1.58 (m, 2H), 1.30-1.18 (m, 1H), 1.06-0.91 (m, 7H). LC-MS (ESI): m/z [M+H]⁺=911.54.

### Example 139: (1R,3S)-3-(3-((2-((4R)-1-((1-(1-(2,6-dioxopiperidin-3-yl)-3-methyl-2-oxo-2,3-dihydro-1H-benzo[d]imidazol-5-yl)piperidin-4-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl isopropylcarbamate

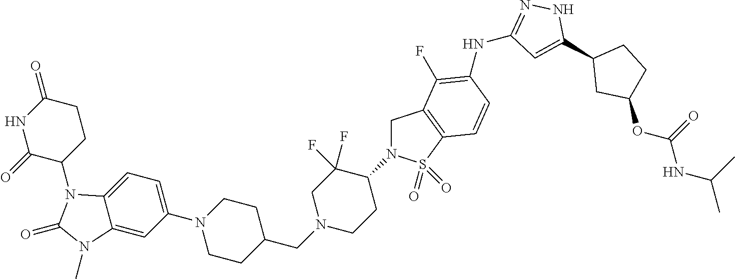 [SMILES: CC(C)NC(=O)O[C@@H]1CC[C@H](c2cc(Nc3ccc4c(c3F)CN([C@@H]3CCN(CC5CCN(c6ccc7c(c6)n(C)c(=O)n7C6CCC(=O)NC6=O)CC5)CC3(F)F)S4(=O)=O)n[nH]2)C1]

The titled compound was synthesized in the procedures similar to Example 36. LC-MS (ESI): m/z [M+H]⁺=911.62; ¹H NMR (500 MHz, DMSO) δ=12.01 (s, 1H), 11.06 (s, 1H), 8.82 (s, 1H), 8.33 (s, 1H), 7.58 (d, J=8.7, 1H), 6.93 (t, J=8.1, 2H), 6.83 (d, J=1.6, 1H), 6.64 (d, J=8.5, 1H), 5.84 (s, 1H), 5.29 (dd, J=12.7, 5.3, 1H), 5.00 (s, 1H), 4.75 (d, J=14.4, 1H), 4.54 (d, J=13.3, 1H), 4.08-3.93 (m, 1H), 3.59 (m, 3H), 3.17 (m, 1H), 3.12-3.02 (m, 1H), 2.95 (m, 1H), 2.88 (m, 1H), 2.70 (m, 1H), 2.66 (m, 1H), 2.63 (m, 3H), 2.60-2.54 (m, 3H), 2.45 (m, 1H), 2.31 (m, 3H), 2.20 (m, 1H), 2.06-1.96 (m, 2H), 1.95-1.86 (m, 1H), 1.80 (m, 3H), 1.72 (m, 2H), 1.63 (m, 2H), 1.25 (m, 2H), 1.03 (m, 6H).

### Example 140: (1R,3S)-3-(3-((2-((4S)-1′-(1-(2,6-dioxopiperidin-3-yl)-3,6-dimethyl-2-oxo-2,3-dihydro-1H-benzo[d]imidazol-5-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

### Step 1: 2,6-bis(benzyloxy)-N-(4-bromo-5-methyl-2-nitrophenyl)pyridin-3-amine

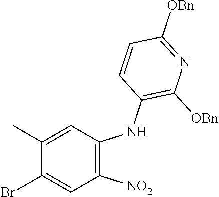 [SMILES: Cc1cc(Nc2ccc(OCc3ccccc3)nc2OCc2ccccc2)c([N+](=O)[O-])cc1Br]

Into a 500-mL 3-necked round-bottom flask purged and maintained with an inert atmosphere of nitrogen were added 2,6-bis(benzyloxy)pyridin-3-amine (8.5 g, 27.78 mmol) in THF 200 mL, add LiHMDS (55.56 mL, 55.56 mmol) dropwised at 0° C. The resulting mixture was stirred for 0.5 h at 0° C. To a solution of 1-bromo-4-fluoro-2-methyl-5-nitrobenzene (6.47 g, 27.78 mmol) in THF 20 mL dropwised at 0° C. The resulting mixture was stirred for 2 h at room temperature. The reaction was quenched with sat NH₄Cl(aq.) at 0° C. The resulting mixture was extracted with EtOAc (600 mL). The organic layers were washed with brine (2×500 mL), dried over anhydrous Na₂SO₄. After filtration, the filtrate was concentrated under reduced pressure. The residue was purified by silica gel column chromatography, eluted with PE/EtOAc (100:0-90:10) to afford 2,6-bis(benzyloxy)-N-(4-bromo-5-methyl-2-nitrophenyl)pyridin-3-amine (10.00 g, 69.36%) as yellow oil.

### Step 2: N1-(2,6-bis(benzyloxy)pyridin-3-yl)-4-bromo-5-methylbenzene-1,2-diamine

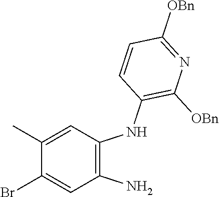 [SMILES: Cc1cc(Nc2ccc(OCc3ccccc3)nc2OCc2ccccc2)c(N)cc1Br]

Into a 500-mL round-bottom flask purged and maintained with an inert atmosphere of nitrogen, were added 2,6-bis(benzyloxy)-N-(4-bromo-5-methyl-2-nitrophenyl)pyridin-3-amine (11.00 g, 21.19 mmol), Fe (3.56 g, 63.58 mmol), NH₄Cl (5.72 g, 105.95 mmol), MeOH (200 mL) and H₂O (20 mL) at room temperature. The resulting mixture was stirred 15 h at 70° C. The resulting mixture was extracted with EtOAc (400 mL). The combined organic layers were washed with brine (2×300 mL), dried over anhydrous Na₂SO₄. After filtration, the filtrate was concentrated under reduced pressure. The residue was purified by silica gel column chromatography, eluted with PE/EtOAc (100:0-80:20) to afford N1-(2,6-bis(benzyloxy)pyridin-3-yl)-4-bromo-5-methylbenzene-1,2-diamine (7.00 g, 67.54%) as yellow solid.

### Step 3: 1-(2,6-bis(benzyloxy)pyridin-3-yl)-5-bromo-6-methyl-1,3-dihydro-2H-benzo[d]imidazol-2-one

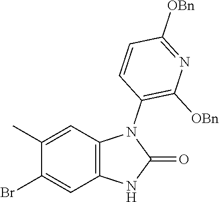 [SMILES: Cc1cc2c(cc1Br)[nH]c(=O)n2-c1ccc(OCc2ccccc2)nc1OCc1ccccc1]

Into a 500-mL round-bottom flask purged and maintained with N₂, were added N1-(2,6-bis(benzyloxy)pyridin-3-yl)-4-bromo-5-methylbenzene-1,2-diamine (7.00 g, 14.31 mmol), CDI (3.48 g, 21.47 mmol) and ACN (200 mL) at room temperature. The resulting mixture was stirred 12 h at 85° C. The resulting mixture was concentrated under reduced pressure. The residue was purified by silica gel column chromatography, eluted with PE/EtOAc (100:0-70:30) to afford 1-(2,6-bis(benzyloxy)pyridin-3-yl)-5-bromo-6-methyl-1,3-dihydro-2H-benzo[d]imidazol-2-one (6.50 g, 88.17%) as yellow solid.

### Step 4: 1-(2,6-bis(benzyloxy)pyridin-3-yl)-5-bromo-3,6-dimethyl-1,3-dihydro-2H-benzo[d]imidazol-2-one

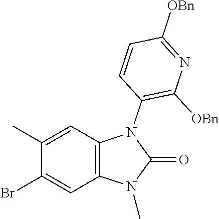 [SMILES: Cc1cc2c(cc1Br)n(C)c(=O)n2-c1ccc(OCc2ccccc2)nc1OCc1ccccc1]

Into a 500-mL round-bottom flask purged and maintained with an inert atmosphere of nitrogen, were added 1-(2,6-bis(benzyloxy)pyridin-3-yl)-5-bromo-6-methyl-1,3-dihydro-2H-benzo[d]imidazol-2-one (6.50 g, 12.62 mmol), CH₃I (2.69 g, 18.93 mmol), K₂CO₃ (3.48 g, 25.24 mmol) and DMF (200 mL) at room temperature. The resulting mixture was stirred 15 h at room temperature. The resulting mixture was extracted with EtOAc (400 mL). The combined organic layers were washed with brine (2×300 mL), dried over anhydrous Na₂SO₄. After filtration, the filtrate was concentrated under reduced pressure. The residue was purified by silica gel column chromatography, eluted with PE/EtOAc (100:0-80:20) to afford 1-(2,6-bis(benzyloxy)pyridin-3-yl)-5-bromo-3,6-dimethyl-1,3-dihydro-2H-benzo[d]imidazol-2-one (6.00 g, 89.86%) as yellow solid.

### Step 5: 1-(2,6-bis(benzyloxy)pyridin-3-yl)-3,6-dimethyl-5-(1,4-dioxa-8-azaspiro[4.5]decan-8-yl)-1,3-dihydro-2H-benzo[d]imidazol-2-one

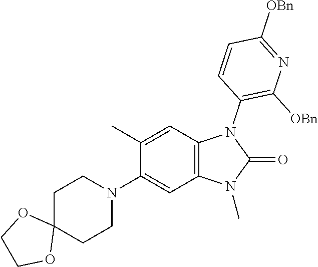 [SMILES: Cc1cc2c(cc1N1CCC3(CC1)OCCO3)n(C)c(=O)n2-c1ccc(OCc2ccccc2)nc1OCc1ccccc1]

Into a 250-mL round-bottom flask purged and maintained with N₂, were added 1-(2,6-bis(benzyloxy)pyridin-3-yl)-5-bromo-3,6-dimethyl-1,3-dihydro-2H-benzo[d]imidazol-2-one (3.00 g, 5.67 mmol), 1,4-dioxa-8-azaspiro[4.5]decane (0.97 g, 6.81 mmol), Cs₂CO₃ (3.69 g, 11.34 mmol), Pd-PEPPSI₂Me-IPENT Cl (238.1 mg, 0.28 mmol) and Dioxane (50 mL) at room temperature. The resulting mixture was stirred 1.5 h at 100° C. After filtration, the filtrate was concentrated under reduced pressure. The residue was purified by silica gel column chromatography, eluted with PE/EtOAc (100:0-70:30) to afford 1-(2,6-bis(benzyloxy)pyridin-3-yl)-3,6-dimethyl-5-(1,4-dioxa-8-azaspiro[4.5]decan-8-yl)-1,3-dihydro-2H-benzo[d]imidazol-2-one (2.40 g, 71.49%) as yellow solid.

### Step 6: 3-(3,6-dimethyl-2-oxo-5-(1,4-dioxa-8-azaspiro[4.5]decan-8-yl)-2,3-dihydro-1H-benzo[d]imidazol-1-yl)piperidine-2,6-dione

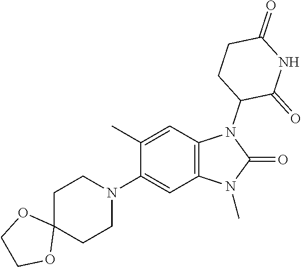 [SMILES: Cc1cc2c(cc1N1CCC3(CC1)OCCO3)n(C)c(=O)n2C1CCC(=O)NC1=O]

Into a 250-mL round-bottom flask purged and maintained with H₂, were added 1-(2,6-bis(benzyloxy)pyridin-3-yl)-3,6-dimethyl-5-(1,4-dioxa-8-azaspiro[4.5]decan-8-yl)-1,3-dihydro-2H-benzo[d]imidazol-2-one (2.30 g, 3.89 mmol), Pd/C (2.30 g) and THF (60 mL) at room temperature. The resulting mixture was stirred 15 h at room temperature. The precipitated solids were collected by filtration and washed with THF (500 mL). After filtration, the filtrate was concentrated under reduced pressure. The residue was purified by silica gel column chromatography, eluted with PE/EtOAc (100:0-50:50) to afford 3-(3,6-dimethyl-2-oxo-5-(1,4-dioxa-8-azaspiro[4.5]decan-8-yl)-2,3-dihydro-1H-benzo[d]imidazol-1-yl)piperidine-2,6-dione (1.50 g, 93.26%) as yellow solid.

### Step 7: 3-(3,6-dimethyl-2-oxo-5-(4-oxopiperidin-1-yl)-2,3-dihydro-1H-benzo[d]imidazol-1-yl)piperidine-2,6-dione

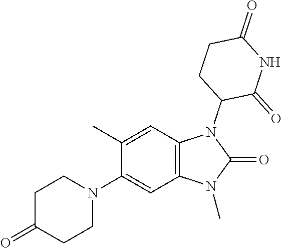 [SMILES: Cc1cc2c(cc1N1CCC(=O)CC1)n(C)c(=O)n2C1CCC(=O)NC1=O]

Into a 100-mL round-bottom flask were added 3-(3,6-dimethyl-2-oxo-5-(1,4-dioxa-8-azaspiro[4.5]decan-8-yl)-2,3-dihydro-1H-benzo[d]imidazol-1-yl)piperidine-2,6-dione (1.80 g, 4.35 mmol), AcOH (20 mL) and H₂O (5 mL) at room temperature. The resulting mixture was stirred 5 h at 80° C. The resulting mixture was concentrated under reduced pressure. The residue was purified by silica gel column chromatography, eluted with PE/EtOAc (100:0-50:50) to afford 3-(3,6-dimethyl-2-oxo-5-(4-oxopiperidin-1-yl)-2,3-dihydro-1H-benzo[d]imidazol-1-yl)piperidine-2,6-dione (1.40 g, 87.03%) as yellow solid.

### Step 8: (1R,3S)-3-(3-((2-((4S)-1′-(1-(2,6-dioxopiperidin-3-yl)-3,6-dimethyl-2-oxo-2,3-dihydro-1H-benzo[d]imidazol-5-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

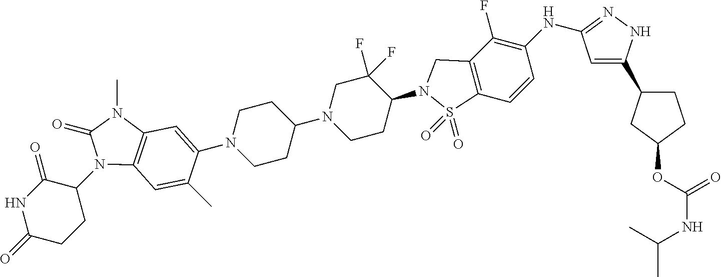 [SMILES: Cc1cc2c(cc1N1CCC(N3CC[C@H](N4Cc5c(ccc(Nc6cc([C@H]7CC[C@@H](OC(=O)NC(C)C)C7)[nH]n6)c5F)S4(=O)=O)C(F)(F)C3)CC1)n(C)c(=O)n2C1CCC(=O)NC1=O]

The titled compound was synthesized in the procedures similar to Example 43. ¹H NMR (500 MHz, DMSO) δ 12.01 (s, 1H), 11.05 (s, 1H), 8.82 (s, 1H), 8.33 (t, J=7.7 Hz, 1H), 7.58 (d, J=8.5 Hz, 1H), 6.7.31-6.82 (m, 3H), 5.84 (s, 1H), 5.30 (dd, J=13.1, 5.2 Hz, 1H), 5.06-4.96 (m, 1H), 4.75 (d, J=14.3 Hz, 1H), 4.54 (d, J=13.6 Hz, 1H), 4.10-3.97 (m, 1H), 3.67-3.50 (m, 1H), 3.26-3.20 (m, 1H), 3.09-3.01 (m, 4H), 2.94-2.78 (m, 3H), 2.76-2.67 (m, 4H), 2.60-2.54 (m, 6H), 2.45-2.43 (m, 1H), 2.26 (s, 3H), 2.20-2.10 (m, 1H), 2.06-1.95 (m, 2H), 1.93-1.89 (m, 1H), 1.88-1.78 (m, 3H), 1.77-1.55 (m, 5H), 1.03 (d, J=5.7 Hz, 6H). LC-MS (ESI): m/z [M+H]⁺=911.4;

### Example 141: (1R,3S)-3-(3-((2-((4R)-1′-(1-(2,6-dioxopiperidin-3-yl)-3,4-dimethyl-2-oxo-2,3-dihydro-1H-benzo[d]imidazol-5-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

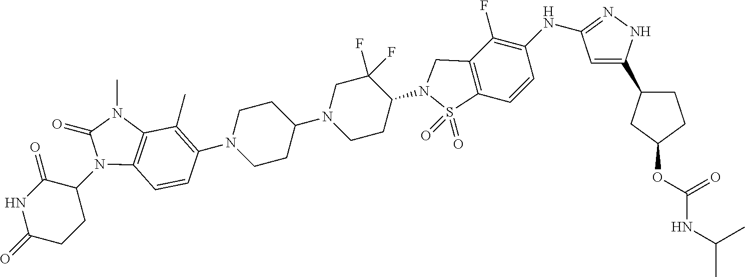 [SMILES: Cc1c(N2CCC(N3CC[C@@H](N4Cc5c(ccc(Nc6cc([C@H]7CC[C@@H](OC(=O)NC(C)C)C7)[nH]n6)c5F)S4(=O)=O)C(F)(F)C3)CC2)ccc2c1n(C)c(=O)n2C1CCC(=O)NC1=O]

The titled compound was synthesized in the procedures similar to Example 43. ¹H NMR (500 MHz, DMSO) δ 12.01 (s, 1H), 11.07 (s, 1H), 8.82 (s, 1H), 8.33 (s, 1H), 7.58 (d, J=8.7 Hz, 1H), 6.94 (d, J=7.3 Hz, 1H), 6.87 (d, J=8.3 Hz, 1H), 6.78 (d, J=8.5 Hz, 1H), 5.84 (s, 1H), 5.31 (dd, J=12.6, 5.3 Hz, 1H), 5.00 (s, 1H), 4.75 (d, J=14.3 Hz, 1H), 4.54 (d, J=13.4 Hz, 1H), 4.04-3.91 (m, 1H), 3.59 (s, 4H), 3.27-3.21 (m, 1H), 3.13-2.96 (m, 4H), 2.92-2.65 (m, 4H), 2.63-2.55 (m, 6H), 2.17-1.59 (m, 13H), 1.07-0.98 (m, 6H). LC-MS (ESI): m/z [M+H]⁺=911.58.

### Example 142: (1R,3S)-3-(3-((2-((4S)-1′-(1-(2,6-dioxopiperidin-3-yl)-3,4-dimethyl-2-oxo-2,3-dihydro-1H-benzo[d]imidazol-5-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

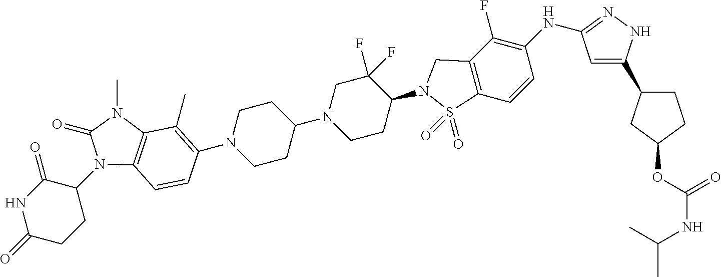 [SMILES: Cc1c(N2CCC(N3CC[C@H](N4Cc5c(ccc(Nc6cc([C@H]7CC[C@@H](OC(=O)NC(C)C)C7)[nH]n6)c5F)S4(=O)=O)C(F)(F)C3)CC2)ccc2c1n(C)c(=O)n2C1CCC(=O)NC1=O]

The titled compound was synthesized in the procedures similar to Example 141. ¹H NMR (500 MHz, DMSO) δ 12.01 (s, 1H), 11.05 (s, 1H), 8.82 (s, 1H), 8.33 (t, J=7.7 Hz, 1H), 7.58 (d, J=8.5 Hz, 1H), 6.7.32-6.81 (m, 3H), 5.84 (s, 1H), 5.30 (dd, J=13.1, 5.2 Hz, 1H), 5.06-4.96 (m, 1H), 4.75 (d, J=14.3 Hz, 1H), 4.54 (d, J=13.6 Hz, 1H), 4.10-3.97 (m, 1H), 3.67-3.50 (m, 1H), 3.26-3.20 (m, 1H), 3.09-3.01 (m, 4H), 2.94-2.78 (m, 3H), 2.76-2.67 (m, 4H), 2.60-2.54 (m, 6H), 2.45-2.43 (m, 1H), 2.26 (s, 3H), 2.20-2.10 (m, 1H), 2.06-1.95 (m, 2H), 1.93-1.89 (m, 1H), 1.88-1.78 (m, 3H), 1.77-1.55 (m, 5H), 1.03 (d, J=5.7 Hz, 6H). LC-MS (ESI): m/z [M+H]⁺=911.4;

### Example 143: (1R,3S)-3-(3-((2-((4S)-1-(1-(1-(2,6-dioxopiperidin-3-yl)-7-fluoro-3-methyl-2-oxo-2,3-dihydro-1H-benzo[d]imidazol-5-yl)azetidin-3-yl)-3,3-difluoropiperidin-4-yl)-4-fluoro-1,1-dioxido-2,3-dihydrobenzo[d]isothiazol-5-yl)amino)-1H-pyrazol-5-yl)cyclopentyl isopropylcarbamate

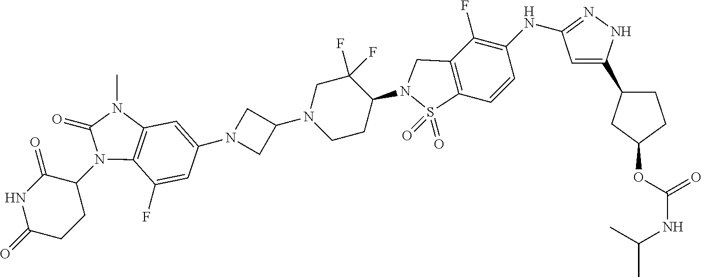 [SMILES: CC(C)NC(=O)O[C@@H]1CC[C@H](c2cc(Nc3ccc4c(c3F)CN([C@H]3CCN(C5CN(c6cc(F)c7c(c6)n(C)c(=O)n7C6CCC(=O)NC6=O)C5)CC3(F)F)S4(=O)=O)n[nH]2)C1]

### Step 1: 3-(7-fluoro-3-methyl-2-oxo-5-(3-oxoazetidin-1-yl)-2,3-dihydro-1H-benzo[d]imidazol-1-yl)piperidine-2,6-dione

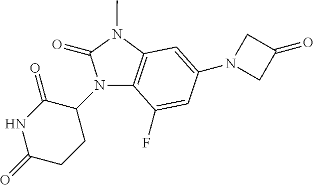 [SMILES: Cn1c(=O)n(C2CCC(=O)NC2=O)c2c(F)cc(N3CC(=O)C3)cc21]

The titled compound was synthesized in the procedures similar to Example 60. LC-MS (ESI): m/z [M+H]⁺=347.3.

### Step 2: (1R,3S)-3-(3-((2-((4S)-1-(1-(1-(2,6-dioxopiperidin-3-yl)-7-fluoro-3-methyl-2-oxo-2,3-dihydro-1H-benzo[d]imidazol-5-yl)azetidin-3-yl)-3,3-difluoropiperidin-4-yl)-4-fluoro-1,1-dioxido-2,3-dihydrobenzo[d]isothiazol-5-yl)amino)-1H-pyrazol-5-yl)cyclopentyl isopropylcarbamate

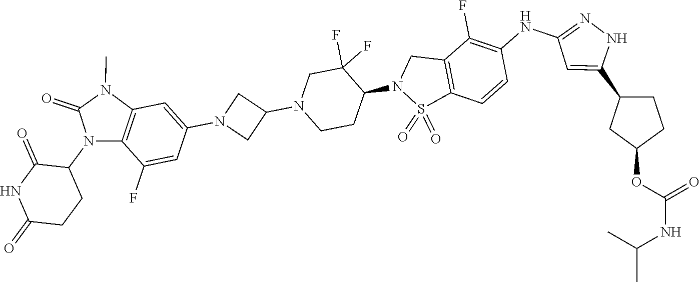 [SMILES: CC(C)NC(=O)O[C@@H]1CC[C@H](c2cc(Nc3ccc4c(c3F)CN([C@H]3CCN(C5CN(c6cc(F)c7c(c6)n(C)c(=O)n7C6CCC(=O)NC6=O)C5)CC3(F)F)S4(=O)=O)n[nH]2)C1]

The titled compound was synthesized in the procedures similar to Example 60. ¹H NMR (500 MHz, DMSO) δ 12.01 (s, 1H), 11.07 (s, 1H), 8.81 (s, 1H), 8.33 (t, J=8.3 Hz, 1H), 7.58 (d, J=8.7 Hz, 1H), 6.95 (s, 1H), 6.20 (s, 1H), 6.03 (d, J=13.1 Hz, 1H), 5.84 (s, 1H), 5.42 (s, 1H), 5.06-4.96 (m, 1H), 4.73 (d, J=14.4 Hz, 1H), 4.52 (d, J=13.6 Hz, 1H), 4.14-4.01 (m, 1H), 3.97-3.91 (m, 2H), 3.64-3.54 (m, 3H), 3.51-3.47 (m, 1H), 3.15-2.89 (m, 4H), 2.59-2.54 (m, 7H), 2.28-2.14 (m, 2H), 2.13-1.98 (m, 2H), 1.96-1.81 (m, 2H), 1.78-1.52 (m, 3H), 1.03 (d, J=6.2 Hz, 6H). LC-MS (ESI): m/z [M+H]⁺=887.4;

### Example 144: (1R,3S)-3-(3-((2-((4R)-1′-(1-(2,6-dioxopiperidin-3-yl)-6-fluoro-3-methyl-2-oxo-2,3-dihydro-1H-benzo[d]imidazol-5-yl)-3,3-dimethyl-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropyl carbamate

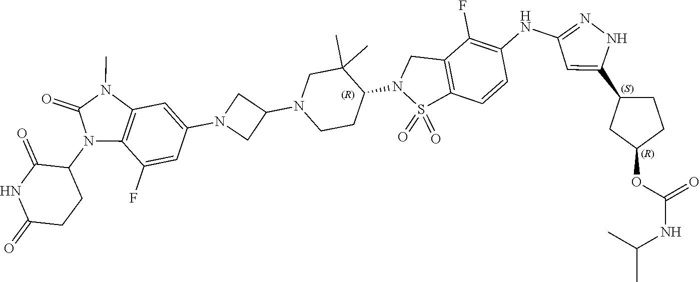

### Step 1: 5-fluoro-1-methyl-6-(1,4-dioxa-8-azaspiro[4.5]decan-8-yl)-1,3-dihydro-2H-benzo[d]imidazol-2-one

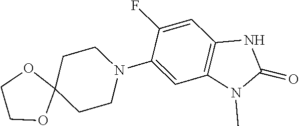 [SMILES: Cn1c(=O)[nH]c2cc(F)c(N3CCC4(CC3)OCCO4)cc21]

To a mixture of 4-(2,4-dioxo-3-((2-(trimethylsilyl)ethoxy)methyl)tetrahydropyrimidin-1(2H)-yl)phenyl trifluoromethanesulfonate (2 g, 8.19 mmol) and 6,6-dimethoxy-2-azaspiro[3.3]heptane, oxalic acid (1.76 g, 12.29 mmol) in andydrous 1,4-dioxane (40 mL) was added Pd₂(dba)₃ (751 mg, 0.819 mmol), RuPhos (765 mg, 1.64 mmol) and LiHMDS (1M in THF, 49 mg, 49 mmol). The reaction mixture was stirred under nitrogen atmosphere at 100° C. for 16 h. quenched with H₂O (10 mL), and concentrated under reduced pressure. The residue was purified by silica gel column chromatography, eluting with PE/EA (1:9) to afford the product (1.9 g, 76% yield). LC-MS (ESI): m/z [M+H]⁺=308.2.

### Step 2: 3-(6-fluoro-3-methyl-2-oxo-5-(1,4-dioxa-8-azaspiro[4.5]decan-8-yl)-2,3-dihydro-1H-benzo[d]imidazol-1-yl)piperidine-2,6-dione

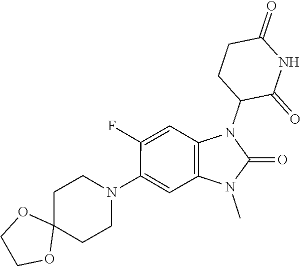 [SMILES: Cn1c(=O)n(C2CCC(=O)NC2=O)c2cc(F)c(N3CCC4(CC3)OCCO4)cc21]

To a solution of 5-fluoro-1-methyl-6-(1,4-dioxa-8-azaspiro[4.5]decan-8-yl)-1,3-dihydro-2H-benzo[d]imidazol-2-one (1.7 g, 5.54 mmol) in THF (20 mL) was was added NaH (443 mg, 11.07 mmol) at 0° C. for 1 h., then 3-bromopiperidine-2,6-dione (1.58 g, 8.31 mmol) was added. the reaction mixture was stirred at 80° C. for 16 h. quenched with H₂O (10 mL), and concentrated under reduced pressure. The residue was purified by silica gel column chromatography, eluting with PE/EA (1:9) to afford the product (1.3 g, 56% yield). LC-MS (ESI): m/z [M+H]⁺=419.2.

### Step 3: 3-(6-fluoro-3-methyl-2-oxo-5-(4-oxopiperidin-1-yl)-2,3-dihydro-1H-benzo[d]imidazol-1-yl)piperidine-2,6-dione

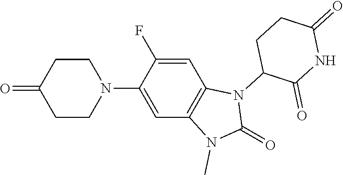 [SMILES: Cn1c(=O)n(C2CCC(=O)NC2=O)c2cc(F)c(N3CCC(=O)CC3)cc21]

To a solution of 3-(6-fluoro-3-methyl-2-oxo-5-(1,4-dioxa-8-azaspiro [4.5]decan-8-yl)-2,3-dihydro-1H-benzo[d]imidazol-1-yl) piperidine-2,6-dione (1.3 g, 3.11 mmol) in H₂SO₄ (20 mL). The reaction mixture was stirred for 1 hours at room temperature. The reaction mixture was quenched with (50 mL) ice water. sat. aq. NaHCO₃ was added to PH>7, the mixture was extracted with EA (100 mL×3). The combined organic phase was washed with brine (200 mL), dried over anhydrous Na2SO4, the mixture was filtered, and the filtrate was concentrated under reduced pressure to afford the product (900 mg, 76% yield). LC-MS (ESI): m/z [M+H]⁺=375.3.

### Step 3: (1R,3S)-3-(3-((2-((4R)-1′-(1-(2,6-dioxopiperidin-3-yl)-6-fluoro-3-methyl-2-oxo-2,3-dihydro-1H-benzo[d]imidazol-5-yl)-3,3-dimethyl-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

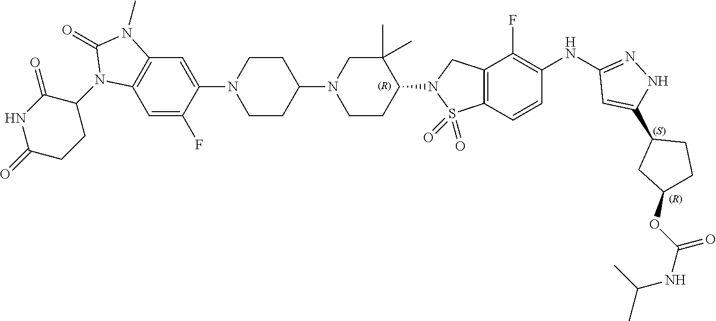

To a solution of (1R,3S)-3-(3-((2-((R)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropyl carbamate (30 mg, 0.055 mmol), 3-(6-fluoro-3-methyl-2-oxo-5-(4-oxopiperidin-1-yl)-2,3-dihydro-1H-benzo[d]imidazol-1-yl)piperidine-2,6-dione (25 mg, 0.084 mmol) and Acetic acid (3.3 mg, 0.084 mmol) in DMA (1.5 mL) was stirred for 30 min, then STAB (24 mg, 0.112 mmol) was added. the reaction mixture was stirred for 16 hours at room tempe-rature. The resulting mixture was concentrated under reduce pressure, the residue was purified by column chromatography on silica gel, eluting with DCM/MeOH (10:1) to afford the product (20 mg, 46%). The residue 20 mg was purified by prep HPLC (Waters XSelect C18: RD-CO-094 column, eluting with a gradient of acetonitrile/water containing 0.1% FA, 20%-40%) to afford the product (10 mg, 22%). LC-MS (ESI): m/z [M+H]⁺=907.63.

¹H NMR (500 MHz, DMSO) δ 11.99 (s, 1H), 11.06 (s, 1H), 8.77 (s, 1H), 8.30 (s, 1H), 7.54 (d, J=8.6 Hz, 1H), 7.12 (d, J=12.0 Hz, 1H), 6.93 (t, J=7.9 Hz, 2H), 5.84 (s, 1H), 5.30 (dd, J=12.9, 5.3 Hz, 1H), 5.00 (s, 1H), 4.54 (s, 2H), 3.67-3.54 (m, 1H), 3.21 (dd, J=12.0, 3.7 Hz, 1H), 3.11-3.00 (m, 2H), 2.91-2.82 (m, 1H), 2.74-2.58 (m, 6H), 2.41-2.27 (m, 2H), 2.17-1.89 (m, 5H), 1.82-1.51 (m, 8H), 1.10 (s, 3H), 1.06-1.00 (m, 6H), 0.95 (s, 3H).

### Example 145: (1R,3S)-3-(3-((2-((4S)-1′-(1-(2,6-dioxopiperidin-3-yl)-6-fluoro-3-methyl-2-oxo-2,3-dihydro-1H-benzo[d]imidazol-5-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

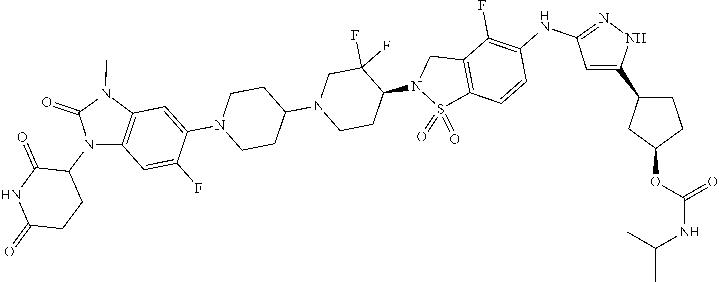 [SMILES: CC(C)NC(=O)O[C@@H]1CC[C@H](c2cc(Nc3ccc4c(c3F)CN([C@H]3CCN(C5CCN(c6cc7c(cc6F)n(C6CCC(=O)NC6=O)c(=O)n7C)CC5)CC3(F)F)S4(=O)=O)n[nH]2)C1]

The titled compound was synthesized in the procedures similar to Example 144. ¹H NMR (500 MHz, DMSO) δ 12.01 (s, 1H), 11.06 (s, 1H), 8.82 (s, 1H), 8.33 (s, 1H), 7.58 (d, J=8.8 Hz, 1H), 7.13 (d, J=12.0 Hz, 1H), 6.94 (d, J=7.3 Hz, 2H), 5.84 (s, 1H), 5.30 (dd, J=12.6, 5.4 Hz, 1H), 5.06-4.96 (m, 1H), 4.75 (d, J=14.4 Hz, 1H), 4.54 (d, J=13.3 Hz, 1H), 4.10-3.97 (m, 1H), 3.67-3.50 (m, 1H), 3.26-3.20 (m, 1H), 3.09-3.01 (m, 4H), 2.94-2.78 (m, 3H), 2.76-2.67 (m, 4H), 2.60-2.54 (m, 6H), 2.45-2.43 (m, 1H), 2.20-2.10 (m, 1H), 2.06-1.95 (m, 2H), 1.93-1.89 (m, 1H), 1.88-1.78 (m, 3H), 1.77-1.55 (m, 5H), 1.03 (d, J=5.7 Hz, 6H). LC-MS (ESI): m/z [M+H]⁺=915.4;

### Example 146: (1R,3S)-3-(3-((2-((4R)-1′-(1-(2,6-dioxopiperidin-3-yl)-6-fluoro-3-methyl-2-oxo-2,3-dihydro-1H-benzo[d]imidazol-5-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

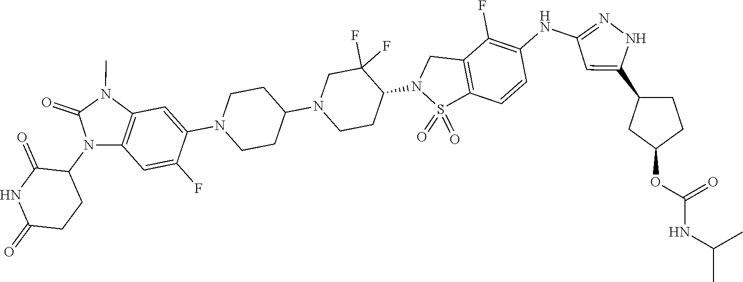 [SMILES: CC(C)NC(=O)O[C@@H]1CC[C@H](c2cc(Nc3ccc4c(c3F)CN([C@@H]3CCN(C5CCN(c6cc7c(cc6F)n(C6CCC(=O)NC6=O)c(=O)n7C)CC5)CC3(F)F)S4(=O)=O)n[nH]2)C1]

The titled compound was synthesized in the procedures similar to Example 144. ¹H NMR (500 MHz, DMSO) δ 11.98 (s, 1H), 11.05 (s, 1H), 8.81 (s, 1H), 8.35-8.29 (m, 1H), 7.58 (d, J=8.6 Hz, 1H), 7.13 (d, J=12.0 Hz, 1H), 6.99-6.91 (m, 2H), 5.84 (s, 1H), 5.42-5.29 (m, 1H), 5.00 (s, 1H), 4.79-4.71 (m, 1H), 4.61-4.39 (m, 1H), 4.04-3.90 (m, 1H), 3.63-3.53 (m, 1H), 3.12 (s, 3H), 2.95-2.50 (m, 14H), 2.19-2.10 (m, 1H), 2.03-1.76 (m, 6H), 1.72-1.51 (m, 5H), 1.09 (d, J=5.7 Hz, 6H). LC-MS (ESI): m/z [M+H]⁺=915.73 Example 147: (1R,3S)-3-(3-((2-((4R)-1-(1-(1-(2,6-dioxopiperidin-3-yl)-3,4-dimethyl-2-oxo-2,3-dihydro-1H-benzo[d]imidazol-5-yl)azetidin-3-yl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropylcarbamate

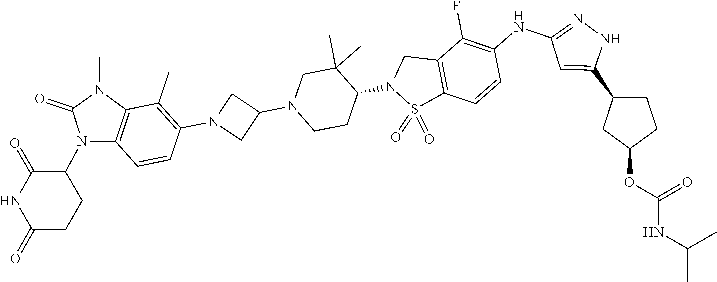 [SMILES: Cc1c(N2CC(N3CC[C@@H](N4Cc5c(ccc(Nc6cc([C@H]7CC[C@@H](OC(=O)NC(C)C)C7)[nH]n6)c5F)S4(=O)=O)C(C)(C)C3)C2)ccc2c1n(C)c(=O)n2C1CCC(=O)NC1=O]

### Step 1: 3-(3,4-dimethyl-2-oxo-5-(3-oxoazetidin-1-yl)-2,3-dihydro-1H-benzo[d]imidazol-1-yl)piperidine-2,6-dione

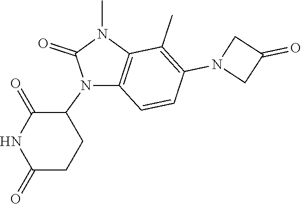 [SMILES: Cc1c(N2CC(=O)C2)ccc2c1n(C)c(=O)n2C1CCC(=O)NC1=O]

The titled compound was synthesized in the procedures similar to Example 45. LC-MS (ESI): m/z [M+H]⁺=343.2

### Step 2: (1R,3S)-3-(3-((2-((4R)-1-(1-(1-(2,6-dioxopiperidin-3-yl)-3,4-dimethyl-2-oxo-2,3-dihydro-1H-benzo[d]imidazol-5-yl)azetidin-3-yl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropylcarbamate

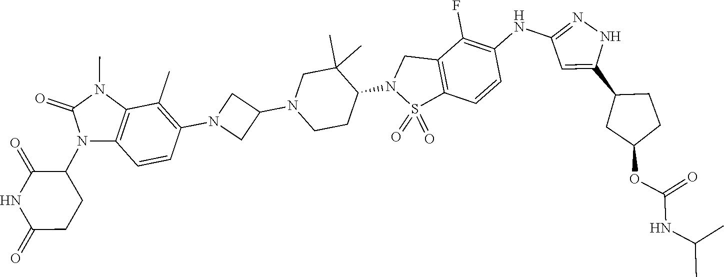 [SMILES: Cc1c(N2CC(N3CC[C@@H](N4Cc5c(ccc(Nc6cc([C@H]7CC[C@@H](OC(=O)NC(C)C)C7)[nH]n6)c5F)S4(=O)=O)C(C)(C)C3)C2)ccc2c1n(C)c(=O)n2C1CCC(=O)NC1=O]

The titled compound was synthesized in the procedures similar to Example 1. ¹H NMR (500 MHz, DMSO) δ 11.98 (s, 1H), 11.06 (s, 1H), 8.76 (s, 1H), 8.30 (s, 1H), 7.54 (d, J=8.7 Hz, 1H), 6.94 (d, J=7.0 Hz, 1H), 6.81 (d, J=8.4 Hz, 1H), 6.33 (d, J=8.4 Hz, 1H), 5.83 (s, 1H), 5.28 (dd, J=12.5, 5.0 Hz, 1H), 5.00 (s, 1H), 4.53 (s, 2H), 3.96-3.87 (m, 2H), 3.55 (s, 4H), 3.48 (d, J=6.2 Hz, 1H), 3.41 (s, 1H), 3.26 (dd, J=12.1, 3.6 Hz, 1H), 3.17-3.02 (m, 2H), 2.95-2.84 (m, 2H), 2.72-2.54 (m, 6H), 2.37 (s, 3H), 2.10-1.90 (m, 4H), 1.80 (d, J=11.3 Hz, 1H), 1.76-1.67 (m, 2H), 1.64-1.50 (m, 2H), 1.09 (s, 3H), 1.07-0.98 (m, 6H), 0.95 (s, 3H). LC-MS (ESI): m/z [M+H]⁺=875.58

### Example 148: (1S,3R)-3-(3-((2-(1-((1-(4-((R)-2,6-dioxopiperidin-3-yl)-3,5-difluorophenyl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

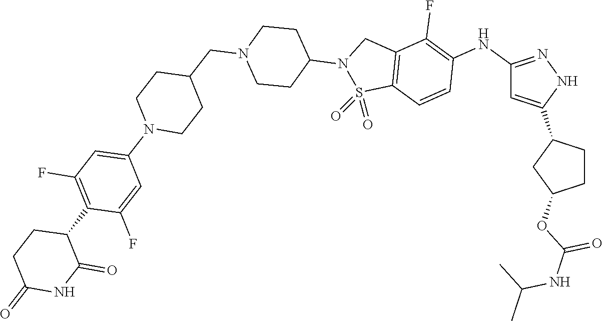 [SMILES: CC(C)NC(=O)O[C@H]1CC[C@@H](c2cc(Nc3ccc4c(c3F)CN(C3CCN(CC5CCN(c6cc(F)c([C@H]7CCC(=O)NC7=O)c(F)c6)CC5)CC3)S4(=O)=O)n[nH]2)C1]

The titled compound was synthesized in the procedures similar to Example 1 in a racemic form, which was further separated by Chiral Prep-HPLC to give Example 148 (100% ee); Retention time: 7.296 min. ¹H NMR (500 MHz, DMSO) δ 11.99 (s, 1H), 10.86 (s, 1H), 8.77 (s, 1H), 8.29 (s, 1H), 7.51 (d, J=8.6 Hz, 1H), 6.94 (d, J=7.3 Hz, 1H), 6.61 (d, J=12.9 Hz, 2H), 5.83 (s, 1H), 5.06-4.96 (m, 1H), 4.52 (s, 2H), 4.04 (dd, J=12.5, 4.9 Hz, 1H), 3.75 (d, J=11.4 Hz, 2H), 3.65-3.53 (m, 1H), 3.47-3.37 (m, 1H), 3.29 (s, 1H), 3.13-3.01 (m, 1H), 2.93-2.86 (m, 2H), 2.83-2.68 (m, 3H), 2.48-2.42 (m, 1H), 2.20-2.14 (m, 2H), 2.08 (dd, J=12.9, 3.6 Hz, 1H), 2.06-1.99 (m, 3H), 1.98-1.80 (m, 6H), 1.80-1.65 (m, 5H), 1.63-1.55 (m, 1H), 1.2-1.07 (m, 2H), 1.03 (d, J=6.4 Hz, 6H). LC-MS (ESI): m/z [M+H]⁺=841.4;

Chiral analytical method: Column: ICHIRALPAK SA 4.6*150 mm 5 μm; Mobile phase: A for MtBE (0.1%2M NH3 MeOH) (%) and B for MeOH:DCM=50:50(%); Gradient: Mobile Phase A: Mobile Phase B=30:70 (v/v); HPLC Equipment: HPLC-Agilent, Back pressure: 100 bar; Column temperature: 35° C.

Chiral Prep-HPLC Condition: CHIRALPAK SA 250*20.0 mml, 5 μm; Mobile phase: A for MtBE (0.1%2M NH3 MeOH) (%) and B for MeOH:DCM=50:50; Gradient: Mobile Phase A: Mobile Phase B=30:70 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 149: (1S,3R)-3-(3-((2-(1-((1-(4-(2,4-dioxotetrahydropyrimidin-1(2H)-yl)phenyl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

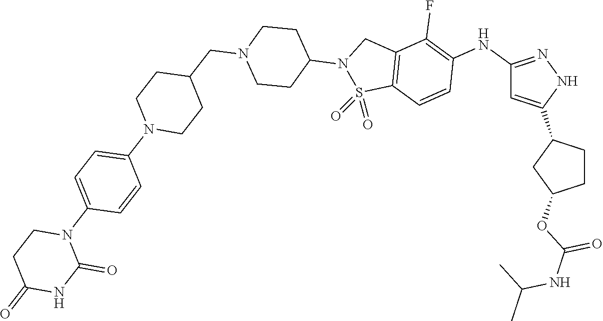 [SMILES: CC(C)NC(=O)O[C@H]1CC[C@@H](c2cc(Nc3ccc4c(c3F)CN(C3CCN(CC5CCN(c6ccc(N7CCC(=O)NC7=O)cc6)CC5)CC3)S4(=O)=O)n[nH]2)C1]

The titled compound was synthesized in the procedures similar to Example 70. ¹H NMR (500 MHz, DMSO) δ 11.99 (s, 1H), 10.25 (s, 1H), 8.77 (s, 1H), 8.29 (d, J=7.6 Hz, 1H), 7.51 (d, J=8.6 Hz, 1H), 7.13 (d, J=8.9 Hz, 2H), 7.05-6.88 (m, 3H), 5.83 (s, 1H), 5.06-4.96 (m, 1H), 4.52 (s, 2H), 3.73-3.65 (m, 4H), 3.63-3.55 (m, 1H), 3.43 (s, 1H), 3.12-3.02 (m, 1H), 2.96-2.86 (m, 2H), 2.72-2.66 (m, 4H), 2.47-2.41 (m, 1H), 2.22-2.14 (m, 2H), 2.10-1.98 (m, 3H), 1.96-0.84 (m, 5H), 1.81-1.55 (m, 6H), 1.25-1.16 (m, 2H), 1.03 (d, J=6.4 Hz, 6H). LC-MS (ESI): m/z [M+H]⁺=806.4;

### Example 150: (1S,3R)-3-(3-((2-((S)-1-((1-(4-(2,4-dioxotetrahydropyrimidin-1(2H)-yl)phenyl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropylcarbamate

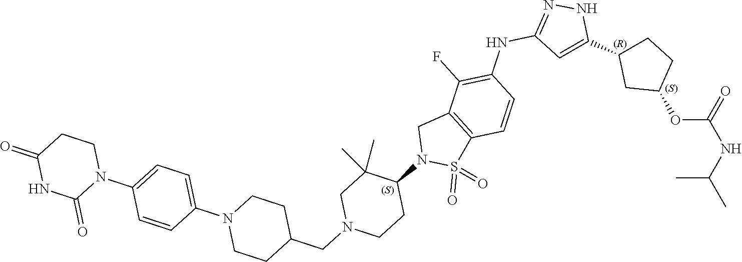

The titled compound was synthesized in the procedures similar to Example 70 in a racemic form, which was further separated by Chiral Prep-HPLC to give Example 150 (100% ee); Retention time: 5.538 min. ¹H NMR (500 MHz, DMSO) δ 11.98 (s, 1H), 10.25 (s, 1H), 8.77 (s, 1H), 8.31 (t, J=7.9 Hz, 1H), 7.54 (d, J=8.6 Hz, 1H), 7.13 (d, J=8.9 Hz, 2H), 7.055-6.87 (m, 3H), 5.83 (s, 1H), 5.00 (s, 1H), 4.54 (s, 2H), 3.69 (t, J=6.7 Hz, 4H), 3.63-3.53 (m, 1H), 3.22 (dd, J=12.2, 3.9 Hz, 1H), 3.11-3.02 (m, 1H), 2.95 (d, J=9.0 Hz, 1H), 2.70-2.64 (m, 4H), 2.23-1.86 (m, 7H), 1.84-1.58 (m, 7H), 1.50 (d, J=9.6 Hz, 1H), 1.26-1.14 (m, 3H), 1.13 (s, 3H), 1.03 (d, J=6.3 Hz, 6H), 0.94 (s, 3H). LC-MS (ESI): m/z [M+H]⁺=834.64

Chiral analytical method: Column: YMC Amylose-SA 4.6*250 mm 5 μm; Mobile phase: A for MtBE and B for MeOH:DCM=50:50 (v/v); Gradient: Mobile Phase A: Mobile Phase B=30:70 (v/v); HPLC Equipment: Agilent 1260 Infinity II (ARD_LC-808); Column temperature: 25° C.

Chiral Prep-HPLC Condition: YMC Amylose-SA 4.6*250 mm 5 μm; Mobile phase: A for MtBE and B for MeOH:DCM=50:50; Gradient: Mobile Phase A: Mobile Phase B=30:70 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 151a and 151b: (1S,3R)-3-(3-((2-((S)-1′-(4-(2,4-dioxotetrahydropyrimidin-1(2H)-yl)phenyl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-((R)-1′-(4-(2,4-dioxotetrahydropyrimidin-1(2H)-yl)phenyl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

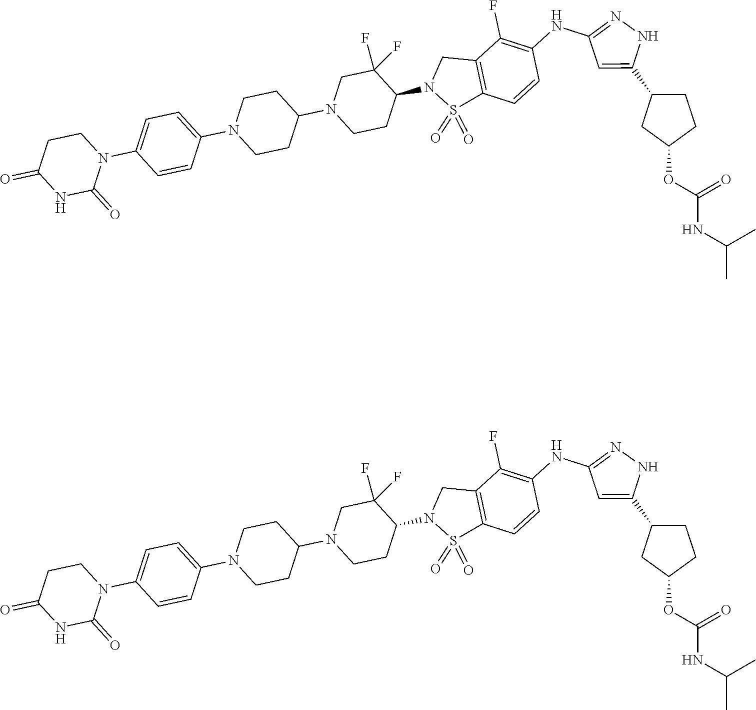 [SMILES: CC(C)NC(=O)O[C@H]1CC[C@@H](c2cc(Nc3ccc4c(c3F)CN([C@H]3CCN(C5CCN(c6ccc(N7CCC(=O)NC7=O)cc6)CC5)CC3(F)F)S4(=O)=O)n[nH]2)C1 ; CC(C)NC(=O)O[C@H]1CC[C@@H](c2cc(Nc3ccc4c(c3F)CN([C@@H]3CCN(C5CCN(c6ccc(N7CCC(=O)NC7=O)cc6)CC5)CC3(F)F)S4(=O)=O)n[nH]2)C1]

The titled compound was synthesized in the procedures similar to Example 5 in a racemic form, which was further separated by Chiral Prep-HPLC to give:

Enantiomer 1 (Example 151a, 100% ee); Retention time: 7.732 min. ¹H NMR (500 MHz, DMSO) δ 12.00 (s, 1H), 10.25 (s, 1H), 8.81 (s, 1H), 8.32 (s, 1H), 7.57 (d, J=8.8 Hz, 1H), 7.14 (d, J=8.4 Hz, 2H), 7.06-6.87 (m, 3H), 5.84 (s, 1H), 5.06-4.96 (m, 1H), 4.73 (d, J=14.3 Hz, 1H), 4.53 (d, J=13.4 Hz, 1H), 4.05-3.90 (m, 1H), 3.79-3.67 (m, 4H), 3.64-3.54 (m, 1H), 3.25-3.15 (m, 1H), 3.13-2.97 (m, 2H), 2.81-2.61 (m, 9H), 2.20-2.08 (m, 1H), 2.06-1.98 (m, 1H), 1.96-1.88 (m, 1H), 1.86-1.66 (m, 5H), 1.63-1.50 (m, 3H), 1.03 (d, J=5.6 Hz, 6H). LC-MS (ESI): m/z [M+H]⁺=828.4;

Enantiomer 2 (Example 151b, 100% ee); Retention time: 8.84 min. ¹H NMR (500 MHz, DMSO) δ 12.00 (s, 1H), 10.25 (s, 1H), 8.81 (s, 1H), 8.32 (s, 1H), 7.57 (d, J=8.8 Hz, 1H), 7.14 (d, J=8.4 Hz, 2H), 7.06-6.87 (m, 3H), 5.84 (s, 1H), 5.06-4.96 (m, 1H), 4.73 (d, J=14.3 Hz, 1H), 4.53 (d, J=13.4 Hz, 1H), 4.05-3.90 (m, 1H), 3.79-3.67 (m, 4H), 3.64-3.54 (m, 1H), 3.25-3.15 (m, 1H), 3.13-2.97 (m, 2H), 2.81-2.61 (m, 9H), 2.20-2.08 (m, 1H), 2.06-1.98 (m, 1H), 1.96-1.88 (m, 1H), 1.86-1.66 (m, 5H), 1.63-1.50 (m, 3H), 1.03 (d, J=5.6 Hz, 6H). LC-MS (ESI): m/z [M+H]⁺=828.4;

Chiral analytical method: Column: ICHIRALPAK IC-5 4.6*150 mm 5 μm; Mobile phase: A for MtBE (0.1%2M NH3 MeOH) (%) and B for MeOH:DCM=50:50(%); Gradient: Mobile Phase A: Mobile Phase B=30:70 (v/v); HPLC Equipment: HPLC-Agilent, Back pressure: 100 bar; Column temperature: 35° C.

Chiral Prep-HPLC Condition: CHIRALPAK IC-5 250*20.0 mml, 5 μm; Mobile phase: A for MtBE (0.1%2M NH3 MeOH) (%) and B for MeOH:DCM=50:50; Gradient: Mobile Phase A: Mobile Phase B=30:70 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 152: (1S,3R)-3-(3-((2-((R)-1-((1-(4-(2,4-dioxotetrahydropyrimidin-1(2H)-yl)phenyl)azetidin-3-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl isopropylcarbamate

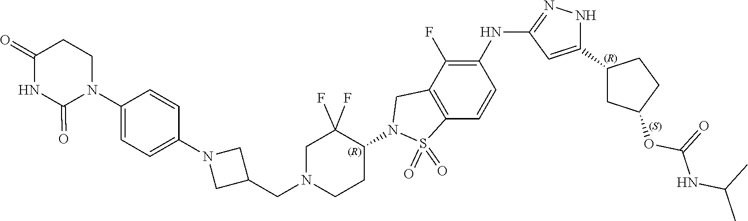

The titled compound was synthesized in the procedures similar to Example 22 in a racemic form, which was further separated by Chiral Prep-HPLC to give Example 152 (100% ee); Retention time: 26.471 min. ¹H NMR (500 MHz, DMSO) δ 12.00 (s, 1H), 10.23 (s, 1H), 8.83 (s, 1H), 8.33 (t, J=8.0 Hz, 1H), 7.58 (d, J=8.7 Hz, 1H), 7.10 (d, J=8.7 Hz, 2H), 6.76 (s, 1H), 6.42 (d, J=8.7 Hz, 2H), 5.84 (s, 1H), 4.98 (s, 1H), 4.64 (dd, J=106.1, 13.7 Hz, 2H), 4.06-3.96 (m, 1H), 3.93 (t, J=7.3 Hz, 2H), 3.67 (t, J=6.7 Hz, 2H), 3.46 (t, J=6.4 Hz, 2H), 3.17 (t, J=9.8 Hz, 1H), 3.11-3.02 (m, 1H), 2.98-2.90 (m, 2H), 2.72 (d, J=7.2 Hz, 2H), 2.67 (t, J=6.7 Hz, 2H), 2.64-2.53 (m, 2H), 2.47-2.44 (m, 1H), 2.42-2.36 (m, 1H), 2.25-2.13 (m, 1H), 2.07-1.98 (m, 1H), 1.96-1.85 (m, 1H), 1.80 (d, J=12.1 Hz, 1H), 1.76-1.65 (m, 2H), 1.58 (s, 1H), 1.06-0.98 (m, 6H). LC-MS (ESI): m/z [M+H]⁺=814.33.

Chiral analytical method: Column: CHIRALPAK IE 4.6*250 mm 5 μm Mobile phase: A for MtBE (0.1%2M NH3 MeOH)(%) and B for MeOH:DCM=50:50 (v/v); Gradient: Mobile Phase A: Mobile Phase B=20:80 (v/v); HPLC Equipment: Agilent 1260 Infinity II (ARD_LC-808); Column temperature: 25° C.

Chiral Prep-HPLC Condition: CHIRALPAK IE 4.6*250 mm 5 μm; Mobile phase: A for MtBE (0.1%2M NH3 MeOH)(%) and B for MeOH:DCM=50:50; Gradient: Mobile Phase A: Mobile Phase B=20:80 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 153: (1S,3R)-3-(3-((2-((S)-1-((1-(4-(2,4-dioxotetrahydropyrimidin-1(2H)-yl)-3-methylphenyl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropylcarbamate

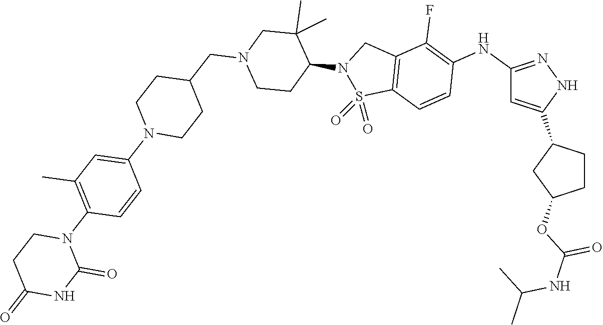 [SMILES: Cc1cc(N2CCC(CN3CC[C@H](N4Cc5c(ccc(Nc6cc([C@@H]7CC[C@H](OC(=O)NC(C)C)C7)[nH]n6)c5F)S4(=O)=O)C(C)(C)C3)CC2)ccc1N1CCC(=O)NC1=O]

The titled compound was synthesized in the procedures similar to Example 76. LC-MS (ESI): m/z [M+H]⁺=848.98; ¹H NMR (500 MHz, DMSO) δ=11.99 (s, 1H), 10.23 (s, 1H), 8.77 (s, 1H), 8.30 (s, 1H), 7.54 (d, J=8.6, 1H), 7.04 (d, J=8.6, 1H), 6.95 (d, J=7.4, 1H), 6.81 (s, 1H), 6.78 (d, J=8.7, 1H), 5.83 (s, 1H), 5.00 (s, 1H), 4.54 (s, 2H), 3.67 (dd, J=16.7, 4.2, 3H), 3.58 (m, 1H), 3.47 (m, 2H), 3.24 (m, 1H), 3.10-3.03 (m, 1H), 2.95 (m, 1H), 2.72 (m, 1H), 2.67 (m, 4H), 2.47-2.44 (m, 1H), 2.15 (m, 2H), 2.12 (m, 3H), 2.04-1.95 (m, 2H), 1.90 (m, 1H), 1.79 (m, 3H), 1.71 (m, 1H), 1.70-1.58 (m, 2H), 1.50 (m, 1H), 1.19 (m, 2H), 1.13 (s, 3H), 1.03 (m, 6H), 0.94 (s, 3H).

### Example 154: (1S,3R)-3-(3-((2-((S)-1-((1-(4-(2,4-dioxotetrahydropyrimidin-1(2H)-yl)-3-fluorophenyl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropylcarbamate

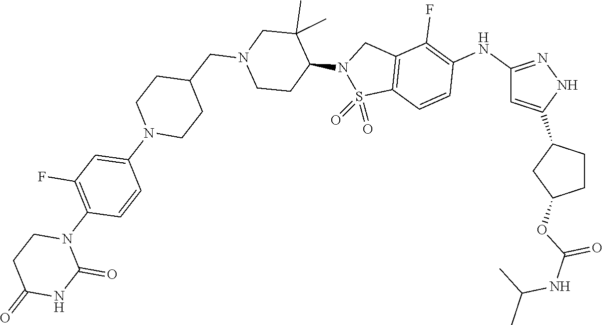 [SMILES: CC(C)NC(=O)O[C@H]1CC[C@@H](c2cc(Nc3ccc4c(c3F)CN([C@H]3CCN(CC5CCN(c6ccc(N7CCC(=O)NC7=O)c(F)c6)CC5)CC3(C)C)S4(=O)=O)n[nH]2)C1]

The titled compound was synthesized in the procedures similar to Example 21. ¹H NMR (500 MHz, DMSO) δ 11.98 (s, 1H), 10.50 (s, 1H), 8.77 (s, 1H), 8.30 (s, 1H), 7.54 (d, J=8.7 Hz, 1H), 6.94 (d, J=7.4 Hz, 1H), 6.71 (d, J=11.9 Hz, 2H), 5.83 (s, 1H), 4.99 (s, 1H), 4.60-4.52 (m, 2H), 3.80 (d, J=9.3 Hz, 2H), 3.66-3.54 (m, 3H), 3.25-3.19 (m, 1H), 3.13-3.01 (m, 1H), 2.99-2.91 (m, 1H), 2.87-2.74 (m, 3H), 2.68 (t, J=6.6 Hz, 2H), 2.45-2.42 (m, 1H), 2.20-1.87 (m, 23.7 Hz, 7H), 1.82-1.68 (m, 6H), 1.64-1.56 (m, 1H), 1.54-1.46 (m, 1H), 1.12 (s, 3H), 1.03 (d, J=6.0 Hz, 6H), 0.93 (s, 3H). LC-MS (ESI): m/z [M+H]⁺=852.4;

### Example 155: (1S,3R)-3-(3-((2-((S)-1-((1-(4-(2,4-dioxotetrahydropyrimidin-1(2H)-yl)-3,5-difluorophenyl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropylcarbamate

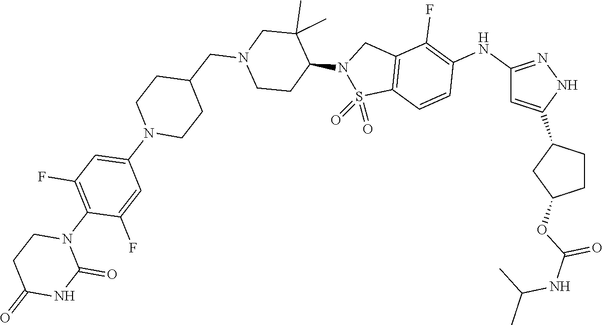 [SMILES: CC(C)NC(=O)O[C@H]1CC[C@@H](c2cc(Nc3ccc4c(c3F)CN([C@H]3CCN(CC5CCN(c6cc(F)c(N7CCC(=O)NC7=O)c(F)c6)CC5)CC3(C)C)S4(=O)=O)n[nH]2)C1]

### Step 1: 1-(4-(2,4-dioxotetrahydropyrimidin-1(2H)-yl)-3,5-difluorophenyl)piperidine-4-carbaldehyde

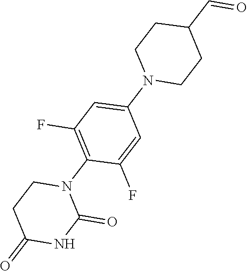 [SMILES: O=CC1CCN(c2cc(F)c(N3CCC(=O)NC3=O)c(F)c2)CC1]

The titled compound was synthesized in the procedures similar to Example 21. LC-MS (ESI): m/z [M+H]⁺=338.4;

### Step 2: (1S,3R)-3-(3-((2-((S)-1-((1-(4-(2,4-dioxotetrahydropyrimidin-1(2H)-yl)-3,5-difluorophenyl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropylcarbamate

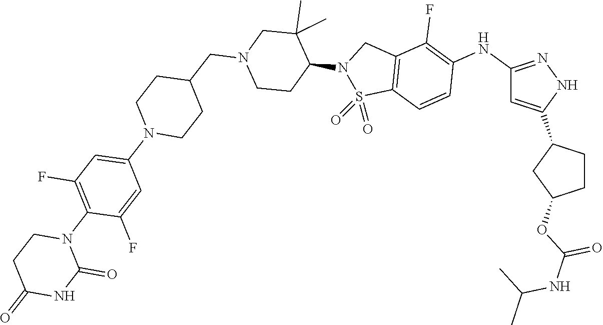 [SMILES: CC(C)NC(=O)O[C@H]1CC[C@@H](c2cc(Nc3ccc4c(c3F)CN([C@H]3CCN(CC5CCN(c6cc(F)c(N7CCC(=O)NC7=O)c(F)c6)CC5)CC3(C)C)S4(=O)=O)n[nH]2)C1]

The titled compound was synthesized in the procedures similar to Example 21. ¹H NMR (500 MHz, DMSO) δ 11.99 (s, 1H), 10.50 (s, 1H), 8.77 (s, 1H), 8.30 (s, 1H), 7.54 (d, J=8.7 Hz, 1H), 6.95 (d, J=7.4 Hz, 1H), 6.71 (d, J=11.9 Hz, 2H), 5.83 (s, 1H), 4.99 (s, 1H), 4.60-4.52 (m, 2H), 3.80 (d, J=9.3 Hz, 2H), 3.66-3.54 (m, 3H), 3.25-3.19 (m, 1H), 3.13-3.01 (m, 1H), 2.99-2.91 (m, 1H), 2.87-2.74 (m, 3H), 2.68 (t, J=6.6 Hz, 2H), 2.45-2.42 (m, 1H), 2.20-1.87 (m, 23.7 Hz, 7H), 1.82-1.68 (m, 6H), 1.64-1.56 (m, 1H), 1.54-1.46 (m, 1H), 1.12 (s, 3H), 1.03 (d, J=6.0 Hz, 6H), 0.93 (s, 3H). LC-MS (ESI): m/z [M+H]⁺=870.4;

### Example 156: (1S,3R)-3-(3-((2-(1-((1-(1-(2,6-dioxopiperidin-3-yl)-3-methyl-2-oxo-2,3-dihydro-1H-benzo[d]imidazol-5-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

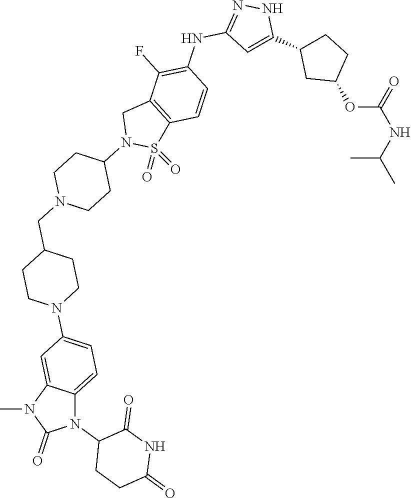 [SMILES: CC(C)NC(=O)O[C@H]1CC[C@@H](c2cc(Nc3ccc4c(c3F)CN(C3CCN(CC5CCN(c6ccc7c(c6)n(C)c(=O)n7C6CCC(=O)NC6=O)CC5)CC3)S4(=O)=O)n[nH]2)C1]

The titled compound was synthesized in the procedures similar to Example 36. ¹H NMR (500 MHz, DMSO-d₆) δ 11.99 (s, 1H), 11.06 (s, 1H), 8.76 (s, 1H), 8.28 (s, 1H), 7.51 (d, J=10 Hz, 1H), 6.98-6.91 (m, 2H), 6.81-6.75 (m, 1H), 6.71-6.58 (m, 1H), 5.83 (s, 1H), 5.33-5.24 (m, 1H), 5.06-4.96 (m, 1H), 4.52 (s, 2H), 3.65-3.52 (m, 2H), 3.48-3.39 (m, 1H), 3.41 (s, 3H), 3.11-3.01 (m, 1H), 2.95-2.88 (m, 3H), 2.71-2.55 (m, 4H), 2.48-2.45 (m, 1H), 2.23-2.08 (m, 2H), 2.07-1.96 (m, 4H), 1.95-1.83 (m, 5H), 1.81-1.50 (m, 6H), 1.38-1.24 (m, 5H), 1.15-0.99 (m, 6H). LC-MS (ESI): m/z [M+H]⁺=875.

### Example 157: (1S,3R)-3-(3-((2-(1-((1-(1-(2,6-dioxopiperidin-3-yl)-4-fluoro-3-methyl-2-oxo-2,3-dihydro-1H-benzo[d]imidazol-5-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

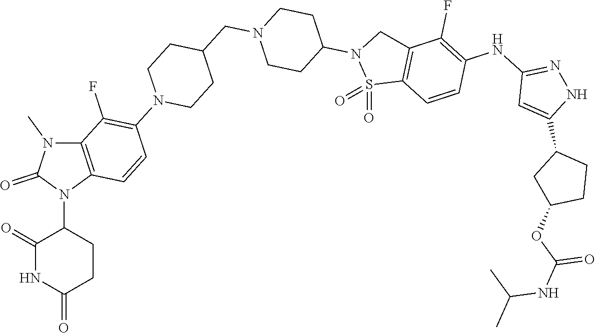 [SMILES: CC(C)NC(=O)O[C@H]1CC[C@@H](c2cc(Nc3ccc4c(c3F)CN(C3CCN(CC5CCN(c6ccc7c(c6F)n(C)c(=O)n7C6CCC(=O)NC6=O)CC5)CC3)S4(=O)=O)n[nH]2)C1]

The titled compound was synthesized in the procedures similar to Example 37. ¹H NMR (500 MHz, DMSO) δ 11.99 (s, 1H), 11.10 (s, 1H), 8.76 (s, 1H), 8.29 (s, 1H), 7.51 (d, J=8.6 Hz, 1H), 5.83 (s, 1H), 5.33 (dd, J=12.7, 5.3 Hz, 1H), 5.06-4.96 (m, 1H), 4.52 (s, 2H), 3.63-3.53 (m, 1H), 3.47 (s, 3H), 3.44-3.39 (m, 1H), 3.23 (d, J=11.3 Hz, 2H), 3.12-3.00 (m, 1H), 2.94-2.84 (m, 3H), 2.75-2.56 (m, 5H), 2.47-2.42 (m, 1H), 2.20 (d, J=6.8 Hz, 2H), 2.07-1.97 (m, 4H), 1.95-1.53 (m, 12H), 1.34-1.23 (m, 2H), 1.03 (d, J=6.4 Hz, 6H). LC-MS (ESI): m/z [M+H]⁺=893.4;

### Example 158: (1S,3R)-3-(3-((2-(1′-(1-(2,6-dioxopiperidin-3-yl)-4-fluoro-3-methyl-2-oxo-2,3-dihydro-1H-benzo[d]imidazol-5-yl)-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

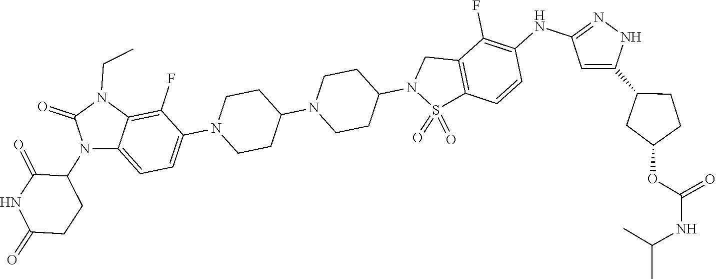 [SMILES: CCn1c(=O)n(C2CCC(=O)NC2=O)c2ccc(N3CCC(N4CCC(N5Cc6c(ccc(Nc7cc([C@@H]8CC[C@H](OC(=O)NC(C)C)C8)[nH]n7)c6F)S5(=O)=O)CC4)CC3)c(F)c21]

The titled compound was synthesized in the procedures similar to Example 45. ¹H NMR (500 MHz, DMSO) δ 12.00 (s, 1H), 11.10 (s, 1H), 8.76 (s, 1H), 8.28 (s, 1H), 7.51 (d, J=8.6 Hz, 1H), 6.94 (d, J=7.4 Hz, 1H), 6.85 (d, J=8.5 Hz, 1H), 6.74 (t, J=8.2 Hz, 1H), 5.83 (s, 1H), 5.33 (dd, J=12.9, 5.3 Hz, 1H), 5.06-4.96 (m, 1H), 4.52 (s, 2H), 3.63-3.53 (m, 1H), 3.47 (s, 3H), 3.44-3.39 (m, 1H), 3.29-3.25 (m, 2H), 3.13-2.96 (m, 3H), 2.92-2.84 (m, 1H), 2.74-2.62 (m, 4H), 2.48-2.40 (m, 2H), 2.38-2.31 (m, 2H), 2.10-1.79 (m, 10H), 1.78-1.55 (m, 5H), 1.03 (d, J=6.4 Hz, 6H). LC-MS (ESI): m/z [M+H]⁺=879.4;

### Example 159: (1S,3R)-3-(3-((2-(1-((1-(3-cyclopropyl-1-(2,6-dioxopiperidin-3-yl)-4-fluoro-2-oxo-2,3-dihydro-1H-benzo[d]imidazol-5-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

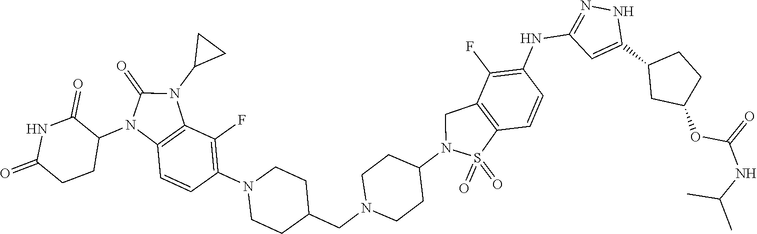 [SMILES: CC(C)NC(=O)O[C@H]1CC[C@@H](c2cc(Nc3ccc4c(c3F)CN(C3CCN(CC5CCN(c6ccc7c(c6F)n(C6CC6)c(=O)n7C6CCC(=O)NC6=O)CC5)CC3)S4(=O)=O)n[nH]2)C1]

The titled compound was synthesized in the procedures similar to Example 38. ¹H NMR (500 MHz, DMSO) δ 11.99 (s, 1H), 11.08 (s, 1H), 8.76 (s, 1H), 8.29 (s, 1H), 7.51 (d, J=8.7 Hz, 1H), 6.94 (d, J=7.3 Hz, 1H), 6.80 (d, J=8.6 Hz, 1H), 6.75 (t, J=8.0 Hz, 1H), 5.83 (s, 1H), 5.28 (dd, J=12.7, 5.3 Hz, 1H), 5.06-4.96 (m, 1H), 4.52 (s, 2H), 3.60-3.54 (m, 1H), 3.49-3.41 (m, 1H), 3.22 (d, J=11.0 Hz, 2H), 3.10-3.02 (m, 2H), 2.96-2.82 (m, 3H), 2.70-2.60 (m, 4H), 2.45-2.42 (m, 1H), 2.21 (d, J=6.7 Hz, 2H), 2.10-1.97 (m, 4H), 1.96-1.78 (m, 8H), 1.76-1.58 (m, 4H), 1.37-1.23 (m, 2H), 1.06-0.94 (m, 10H). LC-MS (ESI): m/z [M+H]+=919.4;

### Example 160: (1S,3R)-3-(3-((2-(1′-(1-(2,6-dioxopiperidin-3-yl)-4-fluoro-2-oxo-3-propyl-2,3-dihydro-1H-benzo[d]imidazol-5-yl)-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

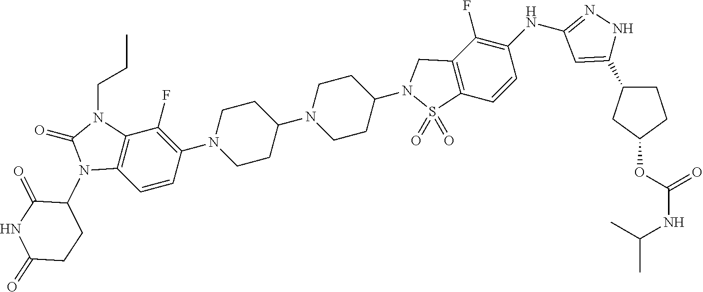 [SMILES: CCCn1c(=O)n(C2CCC(=O)NC2=O)c2ccc(N3CCC(N4CCC(N5Cc6c(ccc(Nc7cc([C@@H]8CC[C@H](OC(=O)NC(C)C)C8)[nH]n7)c6F)S5(=O)=O)CC4)CC3)c(F)c21]

### Step 1: 3-(4-fluoro-2-oxo-5-(4-oxopiperidin-1-yl)-3-propyl-2,3-dihydro-1H-benzo[d]imidazol-1-yl)piperidine-2,6-dione

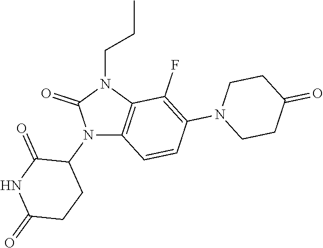 [SMILES: CCCn1c(=O)n(C2CCC(=O)NC2=O)c2ccc(N3CCC(=O)CC3)c(F)c21]

The titled compound was synthesized in the procedures similar to Example 39. LC-MS (ESI): m/z [M+H]⁺=403.4;

### Step 2: (1S,3R)-3-(3-((2-(1′-(1-(2,6-dioxopiperidin-3-yl)-4-fluoro-2-oxo-3-propyl-2,3-dihydro-1H-benzo[d]imidazol-5-yl)-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

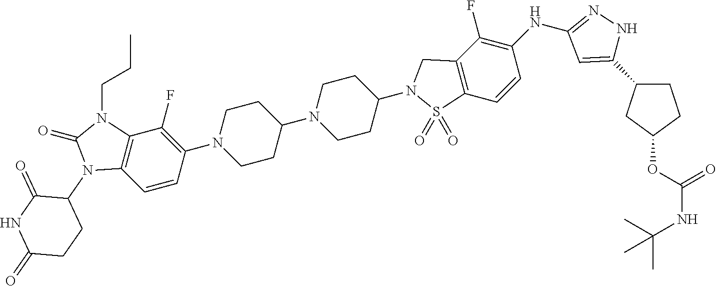 [SMILES: CCCn1c(=O)n(C2CCC(=O)NC2=O)c2ccc(N3CCC(N4CCC(N5Cc6c(ccc(Nc7cc([C@@H]8CC[C@H](OC(=O)NC(C)(C)C)C8)[nH]n7)c6F)S5(=O)=O)CC4)CC3)c(F)c21]

The titled compound was synthesized in the procedures similar to Example 39. ¹H NMR (500 MHz, DMSO) δ 12.00 (s, 1H), 11.09 (s, 1H), 8.77 (s, 1H), 8.30 (s, 1H), 7.51 (d, J=8.6 Hz, 1H), 6.94 (d, J=7.5 Hz, 1H), 6.87 (d, J=8.4 Hz, 1H), 6.75 (t, J=8.2 Hz, 1H), 5.83 (s, 1H), 5.33 (dd, J=12.9, 5.4 Hz, 1H), 5.06-4.96 (m, 1H), 4.52 (s, 2H), 3.90-3.82 (m, 2H), 3.65-3.53-3.52 (m, 1H), 3.52-3.40 (m, 2H), 3.29-3.25 (m, 2H), 3.12-2.98 (m, 3H), 2.94-2.84 (m, 2H), 2.76-2.66 (m, 3H), 2.44-2.40 (m, 1H), 2.09-1.76 (m, 10H), 1.77-1.50 (m, 8H), 1.03 (d, J=6.3 Hz, 6H), 0.87 (t, 3H). LC-MS (ESI): m/z [M+H]⁺=907.4;

### Example 161: (1S,3R)-3-(3-((2-(1-((1-(1-(2,6-dioxopiperidin-3-yl)-3-ethyl-4-fluoro-2-oxo-2,3-dihydro-1H-benzo[d]imidazol-5-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

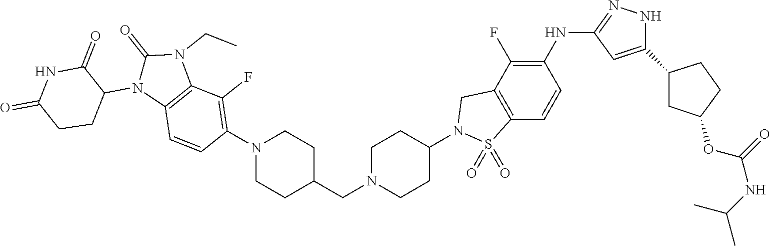 [SMILES: CCn1c(=O)n(C2CCC(=O)NC2=O)c2ccc(N3CCC(CN4CCC(N5Cc6c(ccc(Nc7cc([C@@H]8CC[C@H](OC(=O)NC(C)C)C8)[nH]n7)c6F)S5(=O)=O)CC4)CC3)c(F)c21]

The titled compound was synthesized in the procedures similar to Example 51. ¹H NMR (500 MHz, DMSO) δ 11.99 (s, 1H), 11.10 (s, 1H), 8.76 (s, 1H), 8.29 (s, 1H), 7.51 (d, J=8.6 Hz, 1H), 6.94 (d, J=7.0 Hz, 1H), 6.86 (d, J=8.4 Hz, 1H), 6.75 (t, J=8.2 Hz, 1H), 5.83 (s, 1H), 5.33 (dd, J=12.8, 5.4 Hz, 1H), 5.06-4.96 (m, 1H), 4.52 (s, 2H), 3.98-3.88 (m, 2H), 3.64-3.54 (m, 1H), 3.50-3.40 (m, 1H), 3.23 (d, J=10.8 Hz, 2H), 3.11-3.02 (m, 1H), 2.94-2.84 (m, 3H), 2.75-2.64 (m, 3H), 2.44-2.40 (m, 1H), 2.20 (d, J=7.1 Hz, 2H), 2.08-1.98 (m, 4H), 1.97-1.78 (m, 8H), 1.78-1.67 (m, 2H), 1.66-1.54 (m, 2H), 1.35-1.27 (m, 2H), 1.26-1.22 (m, 3H), 1.03 (d, J=6.3 Hz, 6H). LC-MS (ESI): m/z [M+H]⁺=907.4;

### Example 162: (1S,3R)-3-(3-((2-(1′-(1-(2,6-dioxopiperidin-3-yl)-3-ethyl-4-fluoro-2-oxo-2,3-dihydro-1H-benzo[d]imidazol-5-yl)-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

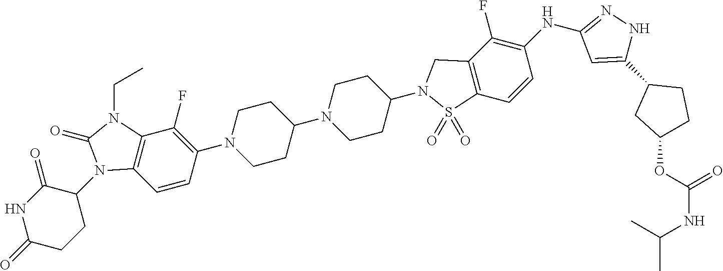 [SMILES: CCn1c(=O)n(C2CCC(=O)NC2=O)c2ccc(N3CCC(N4CCC(N5Cc6c(ccc(Nc7cc([C@@H]8CC[C@H](OC(=O)NC(C)C)C8)[nH]n7)c6F)S5(=O)=O)CC4)CC3)c(F)c21]

The titled compound was synthesized in the procedures similar to Example 39. ¹H NMR (500 MHz, DMSO) δ 12.01 (s, 1H), 11.10 (s, 1H), 8.79 (s, 1H), 8.31 (s, 1H), 7.54 (d, J=8.6 Hz, 1H), 5.84 (s, 1H), 5.34 (dd, J=12.6, 5.1 Hz, 1H), 5.06-4.96 (m, 1H), 4.55 (s, 2H), 3.98-3.90 (m, 2H), 3.65-3.53-3.54 (m, 2H), 3.47-3.36 (m, 3H), 3.30-3.26 (m, 2H), 3.14-3.02 (m, 2H), 2.96-2.84 (m, 2H), 2.76-2.66 (m, 4H), 2.62-2.58 (m, 1H), 2.45-2.41 (m, 1H), 2.03-1.98 (m, 7H), 1.96-1.87 (m, 2H), 1.79-1.66 (m, 3H), 1.65-1.55 (m, 1H), 1.25 (t, 3H), 1.07-0.98 (m, 6H). LC-MS (ESI): m/z [M+H]⁺=893.4;

### Example 163: (1S,3R)-3-(3-((2-(1-((1-(1-(2,6-dioxopiperidin-3-yl)-4-fluoro-3-isopropyl-2-oxo-2,3-dihydro-1H-benzo[d]imidazol-5-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

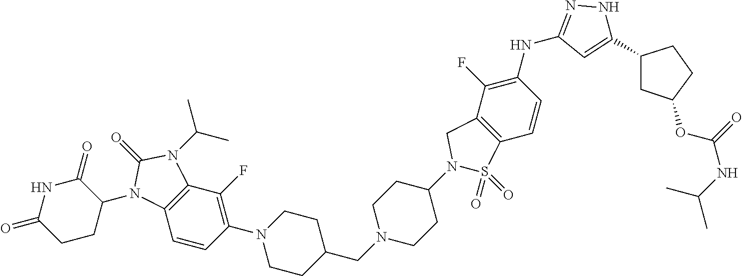 [SMILES: CC(C)NC(=O)O[C@H]1CC[C@@H](c2cc(Nc3ccc4c(c3F)CN(C3CCN(CC5CCN(c6ccc7c(c6F)n(C(C)C)c(=O)n7C6CCC(=O)NC6=O)CC5)CC3)S4(=O)=O)n[nH]2)C1]

The titled compound was synthesized in the procedures similar to Example 40. ¹H NMR (500 MHz, DMSO) δ 11.99 (s, 1H), 11.09 (s, 1H), 8.76 (s, 1H), 8.29 (s, 1H), 7.51 (d, J=8.7 Hz, 1H), 6.94 (d, J=8.0 Hz, 1H), 6.86 (d, J=8.5 Hz, 1H), 6.78 (t, J=8.0 Hz, 1H), 5.83 (s, 1H), 5.33 (dd, J=12.8, 5.5 Hz, 1H), 5.06-4.96 (m, 1H), 4.82-4.72 (m, 1H), 4.52 (s, 2H), 3.63-3.53 (m, 1H), 3.50-3.41 (m, 1H), 3.21 (d, J=10.4 Hz, 2H), 3.012-3.02 (m, 1H), 2.96-2.82 (m, 3H), 2.70-2.64 (m, 3H), 2.45-2.43 (m, 1H), 2.21 (d, J=6.1 Hz, 2H), 2.08-1.98 (m, 4H), 1.92-1.78 (m, 7H), 1.76-1.68 (m, 2H), 1.66-1.54 (m, 2H), 1.42 (d, J=6.9 Hz, 6H), 1.36-1.26 (m, 2H), 1.03 (d, J=6.3 Hz, 6H). LC-MS (ESI): m/z [M+H]⁺=921.4;

### Example 164: (1S,3R)-3-(3-((2-(1′-(1-(2,6-dioxopiperidin-3-yl)-4-fluoro-3-isopropyl-2-oxo-2,3-dihydro-1H-benzo[d]imidazol-5-yl)-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

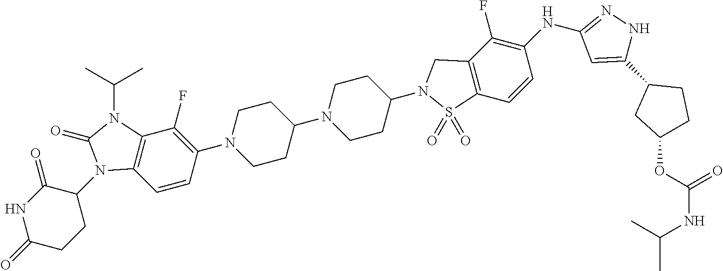 [SMILES: CC(C)NC(=O)O[C@H]1CC[C@@H](c2cc(Nc3ccc4c(c3F)CN(C3CCN(C5CCN(c6ccc7c(c6F)n(C(C)C)c(=O)n7C6CCC(=O)NC6=O)CC5)CC3)S4(=O)=O)n[nH]2)C1]

The titled compound was synthesized in the procedures similar to Example 59. ¹H NMR (500 MHz, DMSO) δ 11.99 (s, 1H), 11.09 (s, 1H), 8.76 (s, 1H), 8.29 (s, 1H), 7.51 (d, J=8.7 Hz, 1H), 6.94 (d, J=8.0 Hz, 1H), 6.86 (d, J=8.5 Hz, 1H), 6.78 (t, J=8.0 Hz, 1H), 5.83 (s, 1H), 5.33 (dd, J=12.8, 5.5 Hz, 1H), 5.06-4.96 (m, 1H), 4.82-4.72 (m, 1H), 4.52 (s, 2H), 3.63-3.53 (m, 1H), 3.50-3.41 (m, 1H), 3.21 (d, J=10.4 Hz, 2H), 3.012-3.02 (m, 1H), 2.96-2.82 (m, 3H), 2.70-2.64 (m, 3H), 2.45-2.43 (m, 1H), 2.21 (d, J=6.1 Hz, 2H), 2.08-1.98 (m, 4H), 1.92-1.78 (m, 9H), 1.76-1.68 (m, 2H), 1.66-1.54 (m, 2H), 1.42 (d, J=6.9 Hz, 6H), 1.36-1.26 (m, 2H), 1.03 (d, J=6.3 Hz, 6H). LC-MS (ESI): m/z [M+H]⁺=907.4;

### Example 165: (1S,3R)-3-(3-((2-(1-((1-(1-(2,6-dioxopiperidin-3-yl)-3-methyl-2-oxo-2,3-dihydro-1H-imidazo[4,5-b]pyridin-5-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

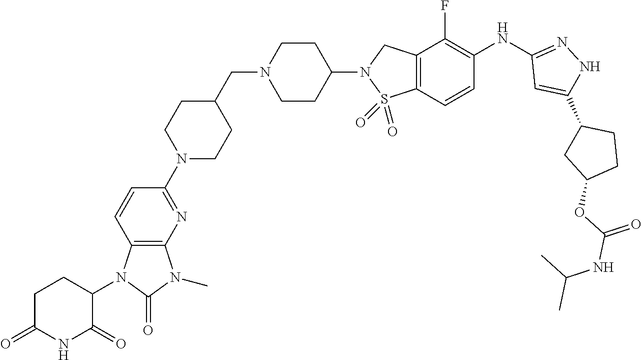 [SMILES: CC(C)NC(=O)O[C@H]1CC[C@@H](c2cc(Nc3ccc4c(c3F)CN(C3CCN(CC5CCN(c6ccc7c(n6)n(C)c(=O)n7C6CCC(=O)NC6=O)CC5)CC3)S4(=O)=O)n[nH]2)C1]

### Step 1: 5-(4-(dimethoxymethyl)piperidin-1-yl)-3-methyl-1,3-dihydro-2H-imidazo[4,5-b]pyridin-2-one

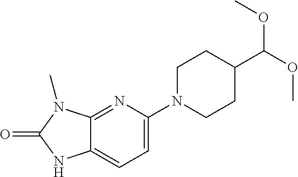 [SMILES: COC(OC)C1CCN(c2ccc3[nH]c(=O)n(C)c3n2)CC1]

To a mixture of 5-chloro-3-methyl-1,3-dihydro-2H-imidazo[4,5-b]pyridin-2-one (500 mg, 2.72 mmol) and 4-(dimethoxymethyl)piperidine (650 mg, 4.09 mmol) in andydrous THF (15 mL) was added Pd₂(dba)₃ (500 mg, 0.54 mmol), RuPhos (508 mg, 1 mmol) and LiHMDS (10.9 mL, 10.9 mmol, 1 M in THF) at 0° C. The reaction mixture was stirred under nitrogen atmosphere at 70° C. for 1 h. The mixture was diluted with EA (100 mL), washed with NH₄Cl aq. (2×50 mL) and brine (50 mL). The organic layer was dried over anhydrous Na₂SO₄, filtered and concentrated under reduced pressure. The residue was purified by silica gel column chromatography, eluting with DCM/MeOH (1:30) to afford the product (420 mg, 50% yield). LC-MS (ESI): m/z [M+H]⁺=307.3.

### Step 2: 3-(5-(4-(dimethoxymethyl)piperidin-1-yl)-3-methyl-2-oxo-2,3-dihydro-1H-imidazo[4,5-b]pyridin-1-yl)piperidine-2,6-dione

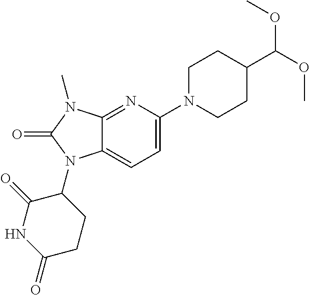 [SMILES: COC(OC)C1CCN(c2ccc3c(n2)n(C)c(=O)n3C2CCC(=O)NC2=O)CC1]

To a stirring solution of 5-(4-(dimethoxymethyl)piperidin-1-yl)-3-methyl-1,3-dihydro-2H-imidazo[4,5-b]pyridin-2-one (420 mg, 1.37 mmol) and 3-bromopiperidine-2,6-dione (526 mg, 2.74 mmol) in THF (10 mL) was added NaH (220 mg, 5.48 mmol, 60% in mineral oil) slowly at 0° C. The resulting solution was heated to 70° C. for 2 h. The solution was cooled to 0° C., quenched with NH₄Cl aq. (50 mL). The mixture was extracted with DCM (100 mL×3). The combined organic layers were washed with brine (50 mL) and dried over anhydrous Na₂SO₄. After filtration, the filtrate was concentrated under reduced pressure. The residue was purified by silica gel column chromatography, eluting with DCM/MeOH (1:30) to afford the product (390 mg, 68% yield). LC-MS (ESI): m/z [M+H]⁺=418.4.

### Step 3: 1-(1-(2,6-dioxopiperidin-3-yl)-3-methyl-2-oxo-2,3-dihydro-1H-imidazo[4,5-b]pyridin-5-yl)piperidine-4-carbaldehyde

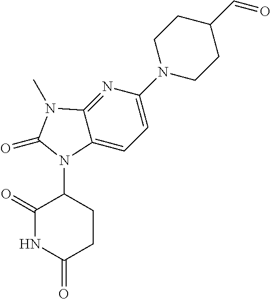 [SMILES: Cn1c(=O)n(C2CCC(=O)NC2=O)c2ccc(N3CCC(C=O)CC3)nc21]

To a mixture of 3-(5-(4-(dimethoxymethyl)piperidin-1-yl)-3-methyl-2-oxo-2,3-dihydro-1H-imidazo[4,5-b]pyridin-1-yl)piperidine-2,6-dione (390 mg, 0.93 mmol) in HCl aq (10 mL, 8 N) was stirred at room temperature for 3 h. The solution was cooled to 0° C., quenched with NaHCO₃ aq. (150 mL). The mixture was extracted with DCM (100 mL×3). The combined organic layers were washed with brine (50 mL) and dried over anhydrous Na₂SO₄. After filtration, the filtrate was concentrated under reduced pressure to afford the product (310 mg, 89% yield). LC-MS (ESI): m/z [M+H]⁺=372.3.

### Step 4: (1S,3R)-3-(3-((2-(1-((1-(1-(2,6-dioxopiperidin-3-yl)-3-methyl-2-oxo-2,3-dihydro-1H-imidazo[4,5-b]pyridin-5-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

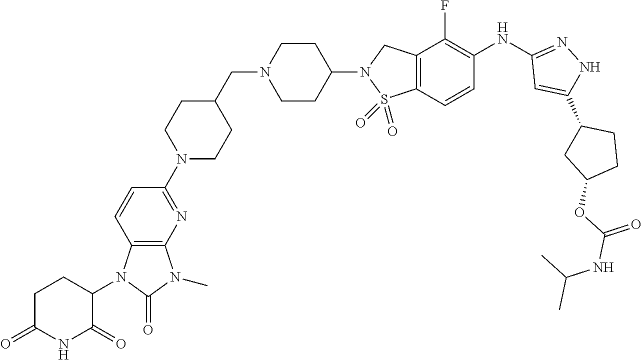 [SMILES: CC(C)NC(=O)O[C@H]1CC[C@@H](c2cc(Nc3ccc4c(c3F)CN(C3CCN(CC5CCN(c6ccc7c(n6)n(C)c(=O)n7C6CCC(=O)NC6=O)CC5)CC3)S4(=O)=O)n[nH]2)C1]

The titled compound was synthesized in the procedures similar to Example 36. ¹H NMR (500 MHz, DMSO) δ 11.99 (s, 1H), 11.07 (s, 1H), 8.77 (s, 1H), 8.29 (s, 1H), 7.51 (d, J=8.6 Hz, 1H), 7.29 (d, J=8.6 Hz, 1H), 6.94 (d, J=7.5 Hz, 1H), 6.43 (d, J=8.7 Hz, 1H), 5.83 (s, 1H), 5.33-5.29 (m, 1H), 5.00 (s, 1H), 4.52 (s, 2H), 4.19-4.17 (m, 2H), 3.66-3.40 (m, 3H), 3.28 (s, 3H), 3.08-3.06 (m, 1H), 2.91-2.85 (m, 3H), 2.77-2.72 (m, 2H), 2.64-2.63 (m, 2H), 2.17-2.16 (m, 2H), 2.03-2.02 (m, 4H), 1.94-1.81 (m, 5H), 1.78-1.71 (m, 5H), 1.60 (s, 1H), 1.14-1.12 (m, 2H), 1.07-1.00 (m, 6H). LC-MS (ESI): m/z [M+H]⁺=876.64.

### Example 166: (1S,3R)-3-(3-((2-(1′-(1-(2,6-dioxopiperidin-3-yl)-3-methyl-2-oxo-2,3-dihydro-1H-imidazo[4,5-b]pyridin-5-yl)-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

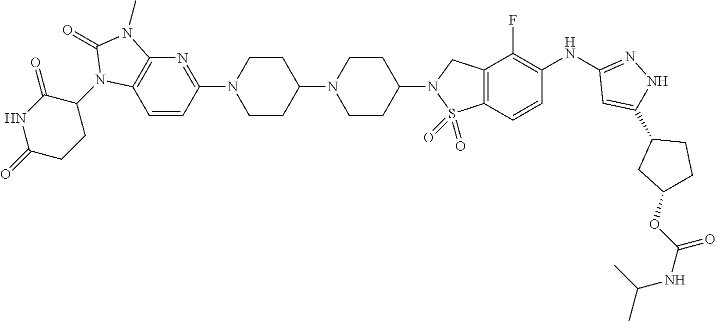 [SMILES: CC(C)NC(=O)O[C@H]1CC[C@@H](c2cc(Nc3ccc4c(c3F)CN(C3CCN(C5CCN(c6ccc7c(n6)n(C)c(=O)n7C6CCC(=O)NC6=O)CC5)CC3)S4(=O)=O)n[nH]2)C1]

### Step 1: 5-(4-(dimethoxymethyl)piperidin-1-yl)-3-methyl-1,3-dihydro-2H-imidazo[4,5-b]pyridin-2-one

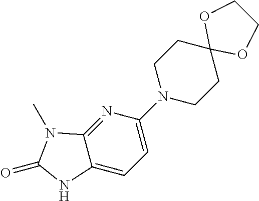 [SMILES: Cn1c(=O)[nH]c2ccc(N3CCC4(CC3)OCCO4)nc21]

To a mixture of 5-chloro-3-methyl-1,3-dihydro-2H-imidazo[4,5-b]pyridin-2-one (500 mg, 2.72 mmol) and 1,4-dioxa-8-azaspiro[4.5]decane (585 mg, 4.09 mmol) in andydrous THF (15 mL) was added Pd2(dba)3 (500 mg, 0.54 mmol), RuPhos (508 mg, 1 mmol) and LiHMDS (10.9 mL, 10.9 mmol, 1 M in THF) at 0° C. The reaction mixture was stirred under nitrogen atmosphere at 70° C. for 1 h. The mixture was diluted with EA (100 mL), washed with NH₄Cl aq. (2×50 mL) and brine (50 mL). The organic layer was dried over anhydrous Na₂SO₄, filtered and concentrated under reduced pressure. The residue was purified by silica gel column chromatography, eluting with DCM/MeOH (1:30) to afford the product (490 mg, 62% yield). LC-MS (ESI): m/z [M+H]⁺=291.5.

### Step 2: 3-(3-methyl-2-oxo-5-(1,4-dioxa-8-azaspiro[4.5]decan-8-yl)-2,3-dihydro-1H-imidazo[4,5-b]pyridin-1-yl)piperidine-2,6-dione

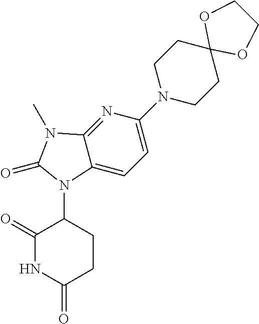 [SMILES: Cn1c(=O)n(C2CCC(=O)NC2=O)c2ccc(N3CCC4(CC3)OCCO4)nc21]

To a stirring solution of 3-methyl-5-(1,4-dioxa-8-azaspiro[4.5]decan-8-yl)-1,3-dihydro-2H-imidazo[4,5-b]pyridin-2-one (490 mg, 1.69 mmol) and 3-bromopiperidine-2,6-dione (648 mg, 3.38 mmol) in THF (12 mL) was added NaH (270 mg, 6.75 mmol, 60% in mineral oil) slowly at 0° C. The resulting solution was heated to 70° C. for 2 h. The solution was cooled to 0° C., quenched with NH₄Cl aq. (50 mL). The mixture was extracted with DCM (100 mL×3). The combined organic layers were washed with brine (50 mL) and dried over anhydrous Na₂SO₄. After filtration, the filtrate was concentrated under reduced pressure. The residue was purified by silica gel column chromatography, eluting with DCM/MeOH (1:30) to afford the product (480 mg, 71% yield). LC-MS (ESI): m/z [M+H]⁺=402.3.

### Step 3: 3-(3-methyl-2-oxo-5-(4-oxopiperidin-1-yl)-2,3-dihydro-1H-imidazo[4,5-b]pyridin-1-yl)piperidine-2,6-dione

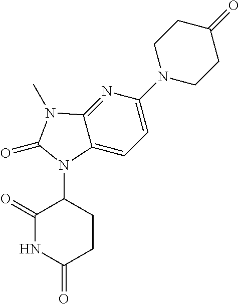 [SMILES: Cn1c(=O)n(C2CCC(=O)NC2=O)c2ccc(N3CCC(=O)CC3)nc21]

To a mixture of 3-(3-methyl-2-oxo-5-(1,4-dioxa-8-azaspiro[4.5]decan-8-yl)-2,3-dihydro-1H-imidazo[4,5-b]pyridin-1-yl)piperidine-2,6-dione (480 mg, 1.2 mmol) in HCl aq (15 mL, 8 N) was stirred at room temperature for 3 h. The solution was cooled to 0° C., quenched with NaHCO₃ aq. (200 mL). The mixture was extracted with DCM (100 mL×3). The combined organic layers were washed with brine (50 mL) and dried over anhydrous Na₂SO₄. After filtration, the filtrate was concentrated under reduced pressure to afford the product (390 mg, 91% yield). LC-MS (ESI): m/z [M+H]⁺=358.4.

### Step 4: (1S,3R)-3-(3-((2-(1′-(1-(2,6-dioxopiperidin-3-yl)-3-methyl-2-oxo-2,3-dihydro-1H-imidazo[4,5-b]pyridin-5-yl)-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

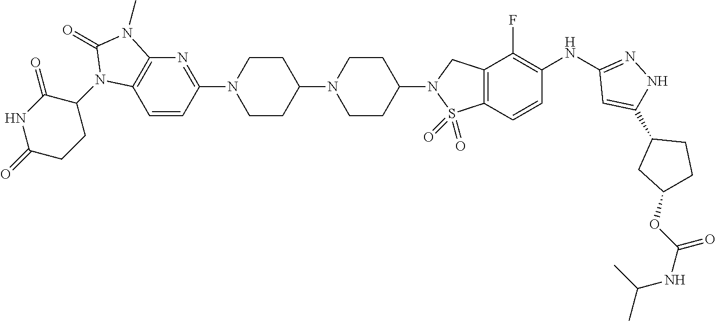 [SMILES: CC(C)NC(=O)O[C@H]1CC[C@@H](c2cc(Nc3ccc4c(c3F)CN(C3CCN(C5CCN(c6ccc7c(n6)n(C)c(=O)n7C6CCC(=O)NC6=O)CC5)CC3)S4(=O)=O)n[nH]2)C1]

The titled compound was synthesized in the procedures similar to Example 39. ¹H NMR (500 MHz, DMSO) δ 11.99 (s, 1H), 11.07 (s, 1H), 8.75 (s, 1H), 8.28 (s, 1H), 7.50 (d, J=8.7 Hz, 1H), 7.30 (d, J=8.6 Hz, 1H), 6.94 (d, J=7.4 Hz, 1H), 6.45 (d, J=8.7 Hz, 1H), 5.83 (s, 1H), 5.33-5.29 (m, 1H), 4.99 (s, 1H), 4.50 (s, 2H), 4.25-4.23 (m, 2H), 3.61-3.54 (m, 1H), 3.42-3.38 (m, 2H), 3.28 (s, 3H), 3.06 (s, 1H), 2.96-2.94 (m, 3H), 2.78-2.59 (m, 4H), 2.30-2.26 (m, 3H), 2.04-2.02 (m, 2H), 1.93-1.91 (m, 3H), 1.83-1.80 (m, 4H), 1.71 (s, 2H), 1.59 (s, 1H), 1.49-1.43 (m, 2H), 1.03-1.02 (m, 6H). LC-MS (ESI): m/z [M+H]⁺=862.6.

### Example 167: (1S,3R)-3-(3-((2-(1-((1-(1-(2,6-dioxopiperidin-3-yl)-4,7-difluoro-3-methyl-2-oxo-2,3-dihydro-1H-benzo[d]imidazol-5-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

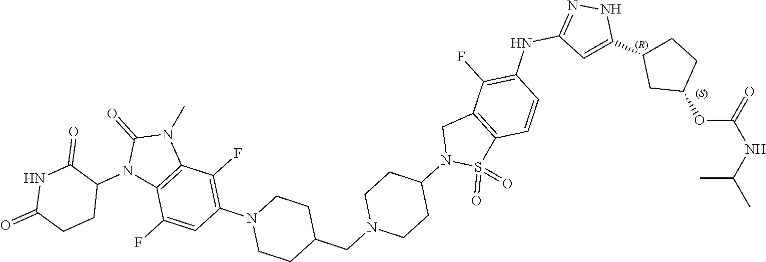

The titled compound was synthesized in the procedures similar to Example 61. ¹H NMR (500 MHz, DMSO) δ 11.99 (s, 1H), 11.11 (s, 1H), 8.76 (s, 1H), 8.28 (s, 1H), 7.51 (d, J=8.6 Hz, 1H), 6.94 (d, J=7.4 Hz, 1H), 6.66 (dd, J=12.4, 5.4 Hz, 1H), 5.83 (s, 1H), 5.46 (d, J=6.9 Hz, 1H), 5.00 (s, 1H), 4.52 (s, 2H), 3.63-3.53 (m, 1H), 3.50 (s, 3H), 3.46-3.39 (m, 2H), 3.25 (s, 3H), 3.10-3.02 (m, 2H), 2.90 (d, J=10.8 Hz, 2H), 2.69-2.60 (m, 3H), 2.33-2.10 (m, 3H), 2.08-1.99 (m, 3H), 1.95-1.70 (m, 9H), 1.62 (d, J=4.1 Hz, 2H), 1.28 (dd, J=10.3 Hz, 2H), 1.07-0.96 (m, 6H). LC-MS (ESI): m/z [M+H]⁺=911.60.

### Example 168: (1S,3R)-3-(3-((2-(1-((1-(1-(2,6-dioxopiperidin-3-yl)-6-fluoro-3-methyl-2-oxo-2,3-dihydro-1H-benzo[d]imidazol-5-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

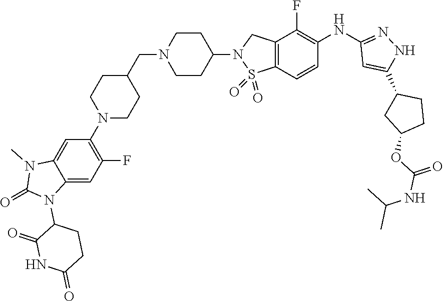 [SMILES: CC(C)NC(=O)O[C@H]1CC[C@@H](c2cc(Nc3ccc4c(c3F)CN(C3CCN(CC5CCN(c6cc7c(cc6F)n(C6CCC(=O)NC6=O)c(=O)n7C)CC5)CC3)S4(=O)=O)n[nH]2)C1]

### Step 1: 1-(1-(2,6-dioxopiperidin-3-yl)-6-fluoro-3-methyl-2-oxo-2,3-dihydro-1H-benzo[d]imidazol-5-yl)piperidine-4-carbaldehyde

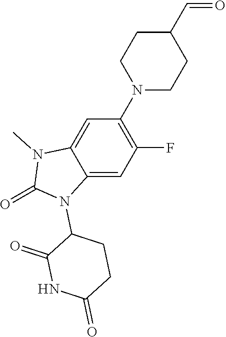 [SMILES: Cn1c(=O)n(C2CCC(=O)NC2=O)c2cc(F)c(N3CCC(C=O)CC3)cc21]

The titled compound was synthesized in the procedures similar to Example 144. LC-MS (ESI): m/z [M+H]⁺=398.3

### Step 2: (1S,3R)-3-(3-((2-(1-((1-(1-(2,6-dioxopiperidin-3-yl)-6-fluoro-3-methyl-2-oxo-2,3-dihydro-1H-benzo[d]imidazol-5-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

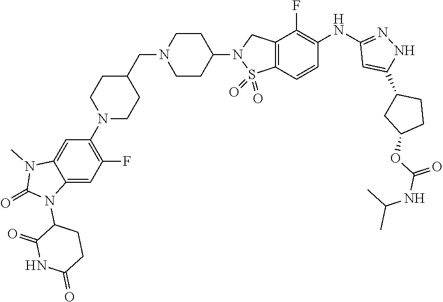 [SMILES: CC(C)NC(=O)O[C@H]1CC[C@@H](c2cc(Nc3ccc4c(c3F)CN(C3CCN(CC5CCN(c6cc7c(cc6F)n(C6CCC(=O)NC6=O)c(=O)n7C)CC5)CC3)S4(=O)=O)n[nH]2)C1]

The titled compound was synthesized in the procedures similar to Example 39. ¹H NMR (500 MHz, DMSO) δ 11.07 (s, 1H), 9.07 (d, J=91.5 Hz, 1H), 8.83 (d, J=21.5 Hz, 1H), 8.30 (d, J=8.2 Hz, 1H), 7.58-7.54 (m, 1H), 7.14 (d, J=12.0 Hz, 1H), 6.93 (d, J=7.3 Hz, 2H), 5.84 (s, 1H), 5.33-5.29 (m, 1H), 5.00 (s, 1H), 4.56 (d, J=5.3 Hz, 2H), 3.83-3.75 (m, 1H), 3.71-3.41 (m, 4H), 3.37-3.23 (m, 5H), 3.16-3.07 (m, 4H), 2.89-2.84 (m, 1H), 2.77-2.66 (m, 3H), 2.63-2.59 (m, 2H), 2.48-2.43 (m, 2H), 2.20 (s, 2H), 2.11-1.84 (m, 6H), 1.72-1.59 (m, 3H), 1.44-1.42 (m, 2H), 1.04-1.02 (m, 6H). LC-MS (ESI): m/z [M+H]⁺=893.58.

### Example 169: (1S,3R)-3-(3-((2-(1′-(1-(2,6-dioxopiperidin-3-yl)-6-fluoro-3-methyl-2-oxo-2,3-dihydro-1H-benzo[d]imidazol-5-yl)-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

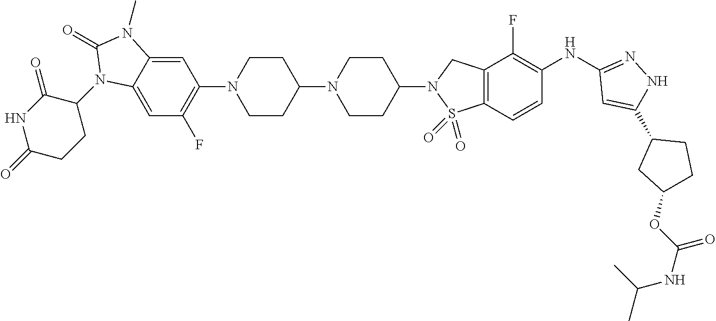 [SMILES: CC(C)NC(=O)O[C@H]1CC[C@@H](c2cc(Nc3ccc4c(c3F)CN(C3CCN(C5CCN(c6cc7c(cc6F)n(C6CCC(=O)NC6=O)c(=O)n7C)CC5)CC3)S4(=O)=O)n[nH]2)C1]

The titled compound was synthesized in the procedures similar to Example 168. ¹H NMR (500 MHz, DMSO) δ 12.00 (s, 1H), 11.06 (s, 1H), 8.76 (s, 1H), 8.30 (s, 1H), 7.51 (d, J=8.6 Hz, 1H), 7.13 (d, J=12.1 Hz, 1H), 6.94 (d, J=7.2 Hz, 2H), 5.83 (s, 1H), 5.32-5.28 (m, 1H), 5.00 (s, 1H), 4.52 (s, 2H), 3.57 (s, 1H), 3.42 (s, 3H), 3.06 (s, 1H), 2.99-2.97 (m, 2H), 2.91-2.82 (m, 1H), 2.73-2.61 (m, 8H), 2.36-2.29 (m, 3H), 2.04-1.93 (m, 5H), 1.85-1.82 (m, 4H), 1.76-1.57 (m, 5H), 1.04-1.02 (m, 6H). LC-MS (ESI): m/z [M+H]⁺=879.59.

### Example 170: (1S,3R)-3-(3-((2-(1-((1-(1-(2,6-dioxopiperidin-3-yl)-3,4-dimethyl-2-oxo-2,3-dihydro-1H-benzo[d]imidazol-5-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

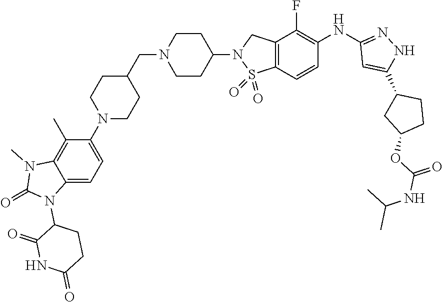 [SMILES: Cc1c(N2CCC(CN3CCC(N4Cc5c(ccc(Nc6cc([C@@H]7CC[C@H](OC(=O)NC(C)C)C7)[nH]n6)c5F)S4(=O)=O)CC3)CC2)ccc2c1n(C)c(=O)n2C1CCC(=O)NC1=O]

The titled compound was synthesized in the procedures similar to Example 42. ¹H NMR (500 MHz, DMSO) δ 11.06 (s, 1H), 8.76 (s, 1H), 8.29 (s, 1H), 7.51 (d, J=8.6 Hz, 1H), 6.94 (d, J=7.7 Hz, 1H), 6.86 (d, J=8.1 Hz, 1H), 6.78 (d, J=8.4 Hz, 1H), 5.83 (s, 1H), 5.31 (dd, J=12.6, 5.2 Hz, 1H), 5.00 (s, 1H), 4.52 (s, 2H), 3.58 (s, 3H), 3.43 (s, 1H), 3.06 (s, 1H), 2.92-2.86 (m, 5H), 2.73-2.53 (m, 10H), 2.22-2.21 (m, 2H), 2.08-1.95 (m, 4H), 1.89 (s, 4H), 1.85-1.66 (m, 5H), 1.60 (s, 2H), 1.30-1.28 (m, 2H), 1.03-1.02 (m, 6H). LC-MS (ESI): m/z [M+H]⁺=889.51.

### Example 171: (1S,3R)-3-(3-((2-(1′-(1-(2,6-dioxopiperidin-3-yl)-3,4-dimethyl-2-oxo-2,3-dihydro-1H-benzo[d]imidazol-5-yl)-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

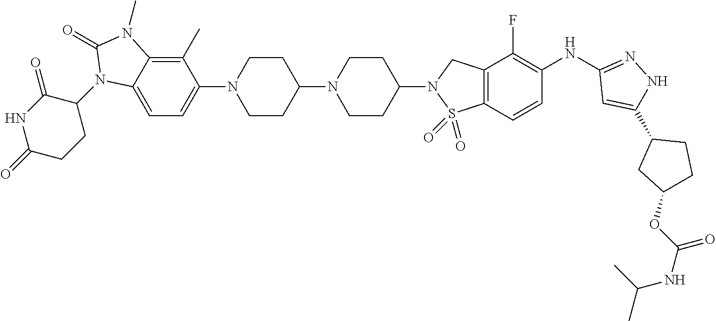 [SMILES: Cc1c(N2CCC(N3CCC(N4Cc5c(ccc(Nc6cc([C@@H]7CC[C@H](OC(=O)NC(C)C)C7)[nH]n6)c5F)S4(=O)=O)CC3)CC2)ccc2c1n(C)c(=O)n2C1CCC(=O)NC1=O]

The titled compound was synthesized in the procedures similar to Example 43. ¹H NMR (500 MHz, DMSO) δ 12.02 (s, 1H), 11.08 (s, 1H), 9.34 (d, J=63.0 Hz, 1H), 8.83 (d, J=21.7 Hz, 1H), 8.30 (d, J=7.6 Hz, 1H), 7.58-7.54 (m, 1H), 6.94-6.88 (m, 2H), 6.80-6.77 (m, 1H), 5.84 (s, 1H), 5.39-5.25 (m, 1H), 5.00 (s, 1H), 4.58 (s, 2H), 3.86 (s, 2H), 3.63 (s, 2H), 3.28-3.22 (m, 3H), 3.07 (s, 3H), 2.95-2.84 (m, 1H), 2.69-2.54 (m, 10H), 2.27-2.25 (m, 1H), 2.18-1.79 (m, 10H), 1.72 (s, 2H), 1.60 (s, 1H), 1.04-1.02 (m, 6H). LC-MS (ESI): m/z [M+H]+=875.53.

### Example 172: (1S,3R)-3-(3-((2-(1-((1-(1-(2,6-dioxopiperidin-3-yl)-2-oxo-2,3-dihydro-1H-benzo[d]imidazol-5-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

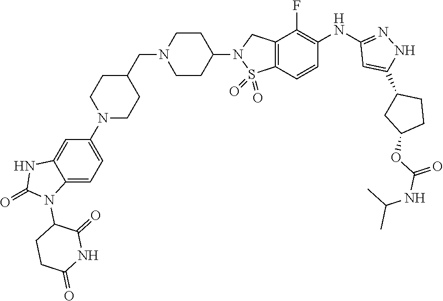 [SMILES: CC(C)NC(=O)O[C@H]1CC[C@@H](c2cc(Nc3ccc4c(c3F)CN(C3CCN(CC5CCN(c6ccc7c(c6)[nH]c(=O)n7C6CCC(=O)NC6=O)CC5)CC3)S4(=O)=O)n[nH]2)C1]

### Step 1: tert-butyl 3-(2,6-bis(benzyloxy)pyridin-3-yl)-6-bromo-2-oxo-2,3-dihydro-1H-benzo[d]imidazole-1-carboxylate

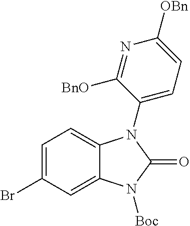 [SMILES: CC(C)(C)OC(=O)n1c(=O)n(-c2ccc(OCc3ccccc3)nc2OCc2ccccc2)c2ccc(Br)cc21]

To a mixture of tert-butyl 6-bromo-2-oxo-2,3-dihydro-1H-benzo[d]imidazole-1-carboxylate (720 mg, 2.3 mmol) and 2,6-bis(benzyloxy)-3-(4,4,5,5-tetramethyl-1,3,2-dioxaborolan-2-yl)pyridine (2.88 g, 6.9 mmol) in andydrous 1,4-dioxane (50 mL) was added Cu(OAc)₂ (835 mg, 4.6 mmol) and pyridine (909 mg, 11.5 mmol). The reaction mixture was stirred under air atmosphere at 80° C. for 16 h. The mixture was filtered, and the filtrate was concentrated under reduced pressure. The residue was purified by silica gel column chromatography, eluting with PE/EA (1:2) to afford the product (710 mg, 51% yield). LC-MS (ESI): m/z [M+H]⁺=602.8.

### Step 2: 1-(2,6-bis(benzyloxy)pyridin-3-yl)-5-(4-(dimethoxymethyl)piperidin-1-yl)-1,3-dihydro-2H-benzo[d]imidazol-2-one

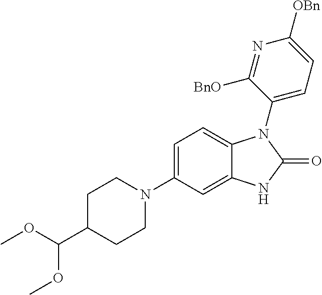 [SMILES: COC(OC)C1CCN(c2ccc3c(c2)[nH]c(=O)n3-c2ccc(OCc3ccccc3)nc2OCc2ccccc2)CC1]

To a mixture of tert-butyl 3-(2,6-bis(benzyloxy)pyridin-3-yl)-6-bromo-2-oxo-2,3-dihydro-1H-benzo[d]imidazole-1-carboxylate (710 mg, 1.18 mmol) and 4-(dimethoxymethyl)piperidine (281 mg, 1.77 mmol) in andydrous THF (20 mL) was added Pd2(dba)3 (215 mg, 0.24 mmol), RuPhos (219 mg, 0.47 mmol) and LiHMDS (5.9 mL, 5.9 mmol, 1 M in THF) at 0° C. The reaction mixture was stirred under nitrogen atmosphere at 70° C. for 1 h. The mixture was diluted with EA (100 mL), washed with NH₄Cl aq. (2×50 mL) and brine (50 mL). The organic layer was dried over anhydrous Na₂SO₄, filtered and concentrated under reduced pressure. The residue was purified by silica gel column chromatography, eluting with DCM/MeOH (1:50) to afford the product (480 mg, 70% yield). LC-MS (ESI): m/z [M+H]+=581.7.

### Step 3: 3-(5-(4-(dimethoxymethyl)piperidin-1-yl)-2-oxo-2,3-dihydro-1H-benzo[d]imidazol-1-yl)piperidine-2,6-dione

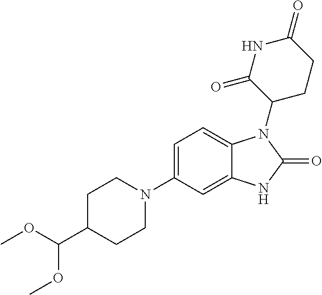 [SMILES: COC(OC)C1CCN(c2ccc3c(c2)[nH]c(=O)n3C2CCC(=O)NC2=O)CC1]

To a solution of 1-(2,6-bis(benzyloxy)pyridin-3-yl)-5-(4-(dimethoxymethyl)piperidin-1-yl)-1,3-dihydro-2H-benzo[d]imidazol-2-one (480 mg, 0.83 mmol) in DMF (10 mL) and i-PrOH (10 mL) was added Pd/C (10%, wet, 500 mg). The suspension was degassed and purged with hydrogen 3 times. The resulting mixture was stirred at 50° C. under hydrogen (0.4 Mp) for 16 h. The mixture was cooled to room temperature and filtered, the filter cake was washed with MeOH (50 mL×3). The filtrate was concentrated under reduced pressure. The residue was purified by silica gel column chromatography, eluting with DCM/MeOH (1:20) to afford the product (160 mg, 48% yield). LC-MS (ESI): m/z [M+H]⁺=403.5.

### Step 4: 1-(1-(2,6-dioxopiperidin-3-yl)-2-oxo-2,3-dihydro-1H-benzo[d]imidazol-5-yl)piperidine-4-carbaldehyde

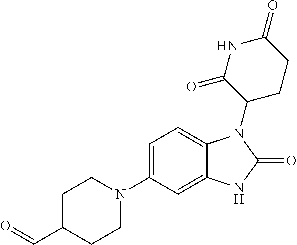 [SMILES: O=CC1CCN(c2ccc3c(c2)[nH]c(=O)n3C2CCC(=O)NC2=O)CC1]

To a mixture of 3-(5-(4-(dimethoxymethyl)piperidin-1-yl)-2-oxo-2,3-dihydro-1H-benzo[d]imidazol-1-yl)piperidine-2,6-dione (120 mg, 0.3 mmol) in HCl aq (5 mL, 8 N) was stirred at room temperature for 3 h. The solution was cooled to 0° C., quenched with NaHCO₃ aq. (50 mL). The mixture was extracted with DCM (50 mL×3). The combined organic layers were washed with brine (50 mL) and dried over anhydrous Na₂SO₄. After filtration, the filtrate was concentrated under reduced pressure to afford the product (90 mg, 91% yield). LC-MS (ESI): m/z [M+H]⁺=357.4.

### Step 5: (1S,3R)-3-(3-((2-(1-((1-(1-(2,6-dioxopiperidin-3-yl)-2-oxo-2,3-dihydro-1H-benzo[d]imidazol-5-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

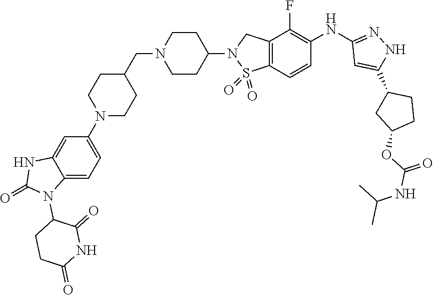 [SMILES: CC(C)NC(=O)O[C@H]1CC[C@@H](c2cc(Nc3ccc4c(c3F)CN(C3CCN(CC5CCN(c6ccc7c(c6)[nH]c(=O)n7C6CCC(=O)NC6=O)CC5)CC3)S4(=O)=O)n[nH]2)C1]

The titled compound was synthesized in the procedures similar to Example 36. ¹H NMR (500 MHz, DMSO) δ 11.99 (s, 1H), 11.04 (s, 1H), 10.74 (s, 1H), 8.76 (s, 1H), 8.29 (s, 1H), 7.51 (d, J=8.7 Hz, 1H), 6.94 (d, J=7.3 Hz, 1H), 6.87 (d, J=8.5 Hz, 1H), 6.60 (d, J=8.0 Hz, 2H), 5.83 (s, 1H), 5.22 (dd, J=12.7, 5.3 Hz, 1H), 5.00 (s, 1H), 4.52 (s, 2H), 3.57 (s, 1H), 3.50-3.48 (m, 3H), 3.43 (s, 3H), 3.06 (s, 1H), 2.91-2.89 (m, 3H), 2.75-2.54 (m, 6H), 2.19-2.17 (m, 2H), 2.03 (s, 2H), 1.89-1.85 (m, 4H), 1.83-1.70 (m, 3H), 1.60 (s, 2H), 1.25-1.22 (m, 2H), 1.04-1.02 (m, 6H). LC-MS (ESI): m/z [M+H]⁺=861.56.

### Example 173: (1S,3R)-3-(3-((2-(1-((1-(1-((S)-2,6-dioxopiperidin-3-yl)-7-fluoro-3-methyl-2-oxo-2,3-dihydro-1H-benzo[d]imidazol-5-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

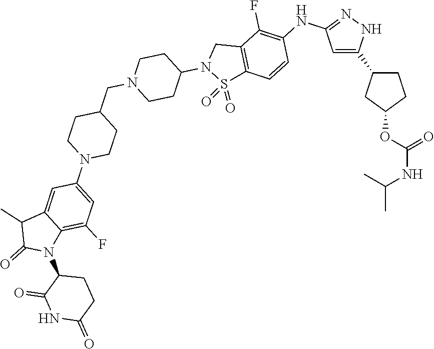 [SMILES: CC(C)NC(=O)O[C@H]1CC[C@@H](c2cc(Nc3ccc4c(c3F)CN(C3CCN(CC5CCN(c6cc(F)c7c(c6)C(C)C(=O)N7[C@H]6CCC(=O)NC6=O)CC5)CC3)S4(=O)=O)n[nH]2)C1]

The titled compound was synthesized in the procedures similar to Example 41. ¹H NMR (500 MHz, DMSO-d₆) δ 11.99 (s, 1H), 11.07 (s, 1H), 8.77 (s, 1H), 8.29 (t, J=5 Hz, 1H), 7.51 (d, J=10 Hz, 1H), 6.94 (d, J=10 Hz, 1H), 6.68 (s, 1H), 6.55-6.49 (m, 1H), 5.83 (s, 1H), 5.51-5.44 (m, 1H), 5.00 (s, 1H), 4.52 (s, 2H), 3.72-3.50 (m, 3H), 3.47-3.39 (m, 1H), 3.32 (s, 3H), 3.09-2.88 (m, 4H), 2.70-2.58 (m, 4H), 2.35-2.15 (m, 3H), 2.11-1.98 (m, 4H), 1.97-1.60 (m, 11H), 1.38-1.19 (m, 2H), 1.04 (s, 3H), 1.02 (s, 3H). LC-MS (ESI): m/z [M+H]⁺=893.

### Example 174: (1S,3R)-3-(3-((2-(1′-(1-(2,6-dioxopiperidin-3-yl)-7-fluoro-3-methyl-2-oxo-2,3-dihydro-1H-benzo[d]imidazol-5-yl)-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

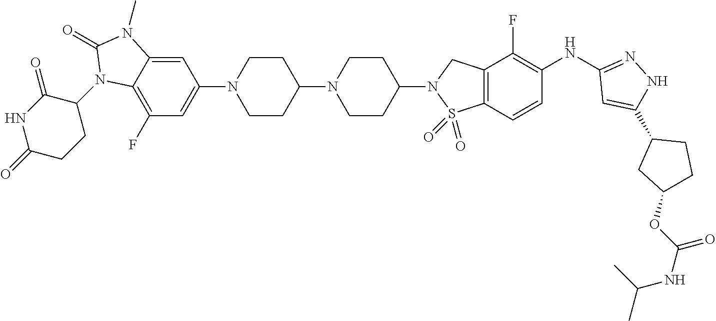 [SMILES: CC(C)NC(=O)O[C@H]1CC[C@@H](c2cc(Nc3ccc4c(c3F)CN(C3CCN(C5CCN(c6cc(F)c7c(c6)n(C)c(=O)n7C6CCC(=O)NC6=O)CC5)CC3)S4(=O)=O)n[nH]2)C1]

The titled compound was synthesized in the procedures similar to Example 60. ¹H NMR (500 MHz, DMSO) δ 12.00 (s, 1H), 11.07 (s, 1H), 8.76 (s, 1H), 8.28 (s, 1H), 7.50 (d, J=8.6 Hz, 1H), 6.94 (d, J=7.9 Hz, 1H), 6.69 (s, 1H), 6.53 (d, J=14.7 Hz, 1H), 5.83 (s, 1H), 5.40 (s, 1H), 5.00 (s, 1H), 4.51 (s, 2H), 3.73-3.71 (m, 3H), 3.60-3.56 (dm, 2H), 3.41 (s, 2H), 3.06 (s, 1H), 2.98-2.96 (m, 2H), 2.68-2.59 (m, 8H), 2.46-2.39 (m, 3H), 2.31-2.26 (m, 2H), 2.07-2.00 (m, 2H), 1.91 (s, 3H), 1.83-1.81 (m, 4H), 1.71 (s, 2H), 1.59-1.52 (m, 3H), 1.03-1.02 (m, 6H). LC-MS (ESI): m/z [M+H]⁺=879.47.

### Example 175: (1S,3R)-3-(3-((2-(1′-(1-((S)-2,6-dioxopiperidin-3-yl)-7-fluoro-3-methyl-2-oxo-2,3-dihydro-1H-benzo[d]imidazol-5-yl)-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

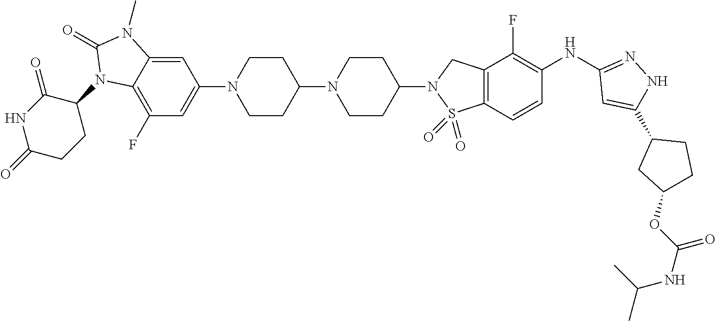 [SMILES: CC(C)NC(=O)O[C@H]1CC[C@@H](c2cc(Nc3ccc4c(c3F)CN(C3CCN(C5CCN(c6cc(F)c7c(c6)n(C)c(=O)n7[C@H]6CCC(=O)NC6=O)CC5)CC3)S4(=O)=O)n[nH]2)C1]

The titled compound was synthesized in the procedures similar to Example 60 in a racemic form, which was further separated by Chiral Prep-HPLC to give Example 175 (100% ee); Retention time: 11.21 min. ¹H NMR (500 MHz, DMSO) δ 12.00 (s, 1H), 11.07 (s, 1H), 8.76 (s, 1H), 8.28 (s, 1H), 7.50 (d, J=8.6 Hz, 1H), 6.94 (d, J=7.9 Hz, 1H), 6.69 (s, 1H), 6.53 (d, J=14.7 Hz, 1H), 5.83 (s, 1H), 5.40 (s, 1H), 5.00 (s, 1H), 4.51 (s, 2H), 3.73-3.71 (m, 3H), 3.60-3.56 (dm, 2H), 3.41 (s, 2H), 3.06 (s, 1H), 2.98-2.96 (m, 2H), 2.68-2.59 (m, 8H), 2.46-2.39 (m, 3H), 2.31-2.26 (m, 2H), 2.07-2.00 (m, 2H), 1.91 (s, 3H), 1.83-1.81 (m, 4H), 1.71 (s, 2H), 1.59-1.52 (m, 3H), 1.03-1.02 (m, 6H). LC-MS (ESI): m/z [M+H]⁺=879.47.

Chiral analytical method: Column: I-Cellulose-5, 4.6 mm×250 mm, 5 um; Mobile phase: A for Hexane and B for EtOH (0.1% 2M NH₃ MeOH); Gradient: Mobile Phase A: Mobile Phase B=30:70 (v/v); HPLC Equipment: HPLC-Agilent, Back pressure: 100 bar; Column temperature: 35° C.

Chiral Prep-HPLC Condition: Column: I-Cellulose-5, 21.2 mm×250 mm, 5 um; Mobile phase: A for Hexane and B for EtOH (0.2% 2M NH₃ MeOH); Gradient: Mobile Phase A: Mobile Phase B=30:70 (v/v); Flow Rate: 20 mL/min, Wave Length: UV 200 nm and 270 nm, Prep-HPLC Equipment: Prep-HPLC-Gilson, Back pressure: 100 bar; Column temperature: 25° C.

### Example 176: (1S,3R)-3-(3-((2-(1-((1-(3-cyclobutyl-1-(2,6-dioxopiperidin-3-yl)-4-fluoro-2-oxo-2,3-dihydro-1H-benzo[d]imidazol-5-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

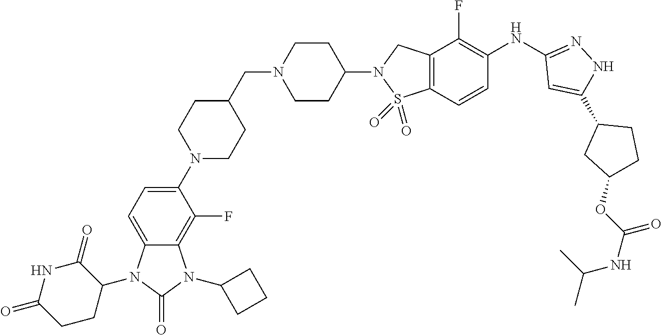 [SMILES: CC(C)NC(=O)O[C@H]1CC[C@@H](c2cc(Nc3ccc4c(c3F)CN(C3CCN(CC5CCN(c6ccc7c(c6F)n(C6CCC6)c(=O)n7C6CCC(=O)NC6=O)CC5)CC3)S4(=O)=O)n[nH]2)C1]

### Step 1: 1-(2,6-bis(benzyloxy)pyridin-3-yl)-3-cyclobutyl-5-(4-(dimethoxymethyl)piperidin-1-yl)-4-fluoro-1,3-dihydro-2H-benzo[d]imidazol-2-one

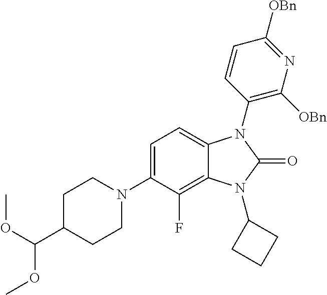 [SMILES: COC(OC)C1CCN(c2ccc3c(c2F)n(C2CCC2)c(=O)n3-c2ccc(OCc3ccccc3)nc2OCc2ccccc2)CC1]

To a solution of 1-(2,6-bis(benzyloxy)pyridin-3-yl)-5-bromo-3-cyclobutyl-4-fluoro-1,3-dihydro-2H-benzo[d]imidazol-2-one (PH-BGUS-H-A631-1) (1.7 g, 2.9 mmol) in Dioxane (30 mL) were added 4-(dimethoxymethyl)piperidine (684 mg, 4.3 mmol), Pd-PEPPSI-IpentCl (132 mg, 0.16 mmol) and Cs₂CO₃ (2 g, 6 mmol) at 100° C. for overnight under N₂ atmosphere. The reaction was concentrated under reduced pressure. The crude was purified by combi-flash PE:EA=100:0 to 65:35 to give the product (800 mg, 42%) as light yellow solid. LCMS (M+H) m/z: 653.

### Step 2: 3-(3-cyclobutyl-5-(4-(dimethoxymethyl)piperidin-1-yl)-4-fluoro-2-oxo-2,3-dihydro-1H-benzo[d]imidazol-1-yl)piperidine-2,6-dione

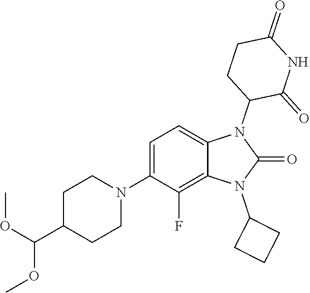 [SMILES: COC(OC)C1CCN(c2ccc3c(c2F)n(C2CCC2)c(=O)n3C2CCC(=O)NC2=O)CC1]

To a solution of 1-(2,6-bis(benzyloxy)pyridin-3-yl)-3-cyclobutyl-5-(4-(dimethoxymethyl)piperidin-1-yl)-4-fluoro-1,3-dihydro-2H-benzo[d]imidazol-2-one (800 mg, 1.2 mmol) in THF (30 mL) was added Pd/C (800 mg) at 50° C. for overnight under H₂ atmosphere. The reaction was filtered. The filtrate was concentrated under vacuum. The residue was without purified to give the product (409.9 mg, 70%) as light brown solid. LCMS (M+H) m/z: 475.20.

### Step 3: 3-(3-cyclobutyl-5-(4-(dimethoxymethyl)piperidin-1-yl)-4-fluoro-2-oxo-2,3-dihydro-1H-benzo[d]imidazol-1-yl)piperidine-2,6-dione

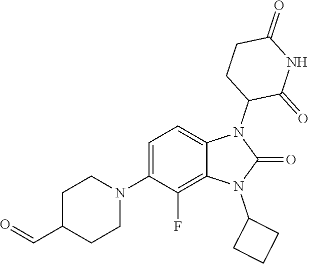 [SMILES: O=CC1CCN(c2ccc3c(c2F)n(C2CCC2)c(=O)n3C2CCC(=O)NC2=O)CC1]

The titled compound was synthesized in the procedures similar to Example 37. LCMS (M+H) m/z: 429.30.

### Step 4: (1S,3R)-3-(3-((2-(1-((1-(3-cyclobutyl-1-(2,6-dioxopiperidin-3-yl)-4-fluoro-2-oxo-2,3-dihydro-1H-benzo[d]imidazol-5-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

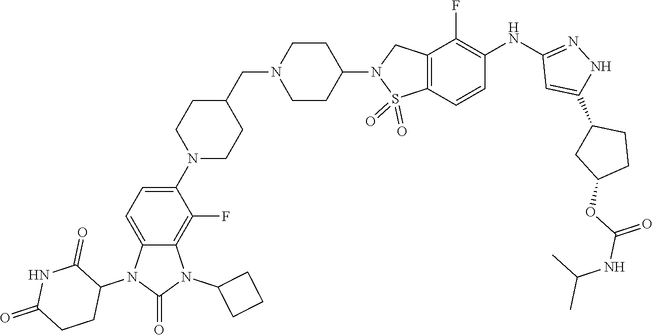 [SMILES: CC(C)NC(=O)O[C@H]1CC[C@@H](c2cc(Nc3ccc4c(c3F)CN(C3CCN(CC5CCN(c6ccc7c(c6F)n(C6CCC6)c(=O)n7C6CCC(=O)NC6=O)CC5)CC3)S4(=O)=O)n[nH]2)C1]

The titled compound was synthesized in the procedures similar to Example 37. ¹H NMR (500 MHz, DMSO) δ 11.99 (s, 1H), 11.10 (s, 1H), 8.76 (s, 1H), 8.29 (s, 1H), 7.51 (d, J=8.6 Hz, 1H), 7.09-6.70 (m, 3H), 5.83 (s, 1H), 5.32 (dd, J=12.8, 5.2 Hz, 1H), 5.17-4.84 (m, 2H), 4.52 (s, 2H), 3.60-3.56 (m, 1H), 3.46-3.40 (m, 1H), 3.24-3.22 (m, 2H), 3.13-3.01 (m, 1H), 2.92-2.84 (m, 3H), 2.80-2.54 (m, 7H), 2.30-2.29 (m, 2H), 2.22-2.21 (m, 2H), 2.06-2.00 (m, 4H), 1.95-1.77 (m, 9H), 1.76-1.67 (m, 2H), 1.62 (s, 2H), 1.35-1.28 (m, 2H), 1.08-0.97 (m, 6H). LC-MS (ESI): m/z [M+H]⁺=933.47.

### Example 177: (1S,3R)-3-(3-((2-(1-((1-(1-(2,6-dioxopiperidin-3-yl)-4-fluoro-3-(oxetan-3-yl)-2-oxo-2,3-dihydro-1H-benzo[d]imidazol-5-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

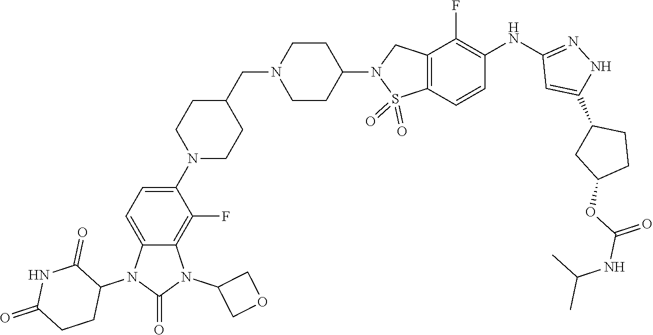 [SMILES: CC(C)NC(=O)O[C@H]1CC[C@@H](c2cc(Nc3ccc4c(c3F)CN(C3CCN(CC5CCN(c6ccc7c(c6F)n(C6COC6)c(=O)n7C6CCC(=O)NC6=O)CC5)CC3)S4(=O)=O)n[nH]2)C1]

### Step 1: 1-(2,6-bis(benzyloxy)pyridin-3-yl)-5-(4-(dimethoxymethyl)piperidin-1-yl)-4-fluoro-3-(oxetan-3-yl)-1,3-dihydro-2H-benzo[d]imidazol-2-one

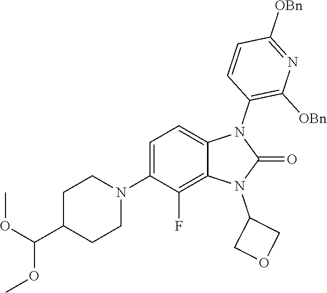 [SMILES: COC(OC)C1CCN(c2ccc3c(c2F)n(C2COC2)c(=O)n3-c2ccc(OCc3ccccc3)nc2OCc2ccccc2)CC1]

To a solution of 1-(2,6-bis(benzyloxy)pyridin-3-yl)-5-bromo-4-fluoro-3-(oxetan-3-yl)-1,3-dihydro-2H-benzo[d]imidazol-2-one (PH-BGUS-H-A633-1) (2.5 g, 4.36 mmol) in dioxane (40 mL) were added 4-(dimethoxymethyl)piperidine (1.04 g, 6.54 mmol), Pd-PEPPSI-IPentCl 2-methylpyridine (183 mg, 0.218 mmol) and Cs₂CO₃ (2.12 g, 6.54 mmol). The resulting solution was stirred for overnight at 100° C. under nitrogen atmosphere. The resulting mixture was concentrated under vacuum. The residue was purified by combi-flash (EtOAc/PE=48:52) to give the product (2 g, 70.4%) as light brown oil

### Step 2: 3-(5-(4-(dimethoxymethyl)piperidin-1-yl)-4-fluoro-3-(oxetan-3-yl)-2-oxo-2,3-dihydro-1H-benzo[d]imidazol-1-yl)piperidine-2,6-dione

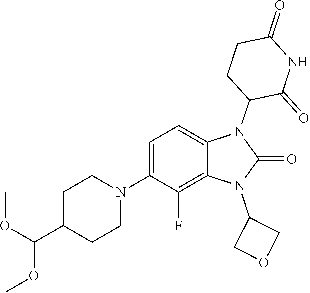 [SMILES: COC(OC)C1CCN(c2ccc3c(c2F)n(C2COC2)c(=O)n3C2CCC(=O)NC2=O)CC1]

To a solution of 1-(2,6-bis(benzyloxy)pyridin-3-yl)-4-fluoro-3-(oxetan-3-yl)-5-(1,4-dioxa-8-azaspiro[4.5]decan-8-yl)-1,3-dihydro-2H-benzo[d]imidazol-2-one (2 g, 6 mmol) in THF (60 mL) was added Pd/C (2 g, 10% wt). The resulting solution was stirred for 12 h at rt under H₂ atmosphere. After filtration, the filtrate was concentrated under vacuum. The residue was the product (1.248 g, 94.4%) as light grey solid. LCMS (ESI) m/e [M+1]⁺=477.

### Step 3: 1-(1-(2,6-dioxopiperidin-3-yl)-4-fluoro-3-(oxetan-3-yl)-2-oxo-2,3-dihydro-1H-benzo[d]imidazol-5-yl)piperidine-4-carbaldehyde

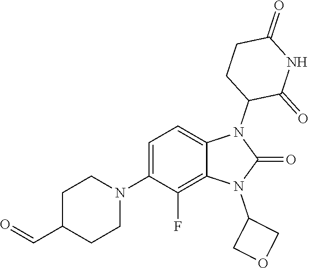 [SMILES: O=CC1CCN(c2ccc3c(c2F)n(C2COC2)c(=O)n3C2CCC(=O)NC2=O)CC1]

The titled compound was synthesized in the procedures similar to Example 37. LC-MS (ESI): m/z [M+H]⁺=431.6

### Step 4: (1S,3R)-3-(3-((2-(1-((1-(1-(2,6-dioxopiperidin-3-yl)-4-fluoro-3-(oxetan-3-yl)-2-oxo-2,3-dihydro-1H-benzo[d]imidazol-5-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

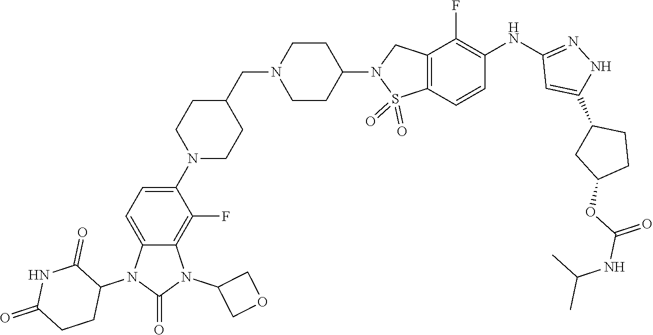 [SMILES: CC(C)NC(=O)O[C@H]1CC[C@@H](c2cc(Nc3ccc4c(c3F)CN(C3CCN(CC5CCN(c6ccc7c(c6F)n(C6COC6)c(=O)n7C6CCC(=O)NC6=O)CC5)CC3)S4(=O)=O)n[nH]2)C1]

The titled compound was synthesized in the procedures similar to Example 37. ¹H NMR (500 MHz, DMSO) δ 11.99 (s, 1H), 11.12 (s, 1H), 8.76 (s, 1H), 8.29 (s, 1H), 7.51 (d, J=8.6 Hz, 1H), 6.95-6.89 (m, 2H), 6.84-6.80 (m, 1H), 5.83 (s, 1H), 5.65-5.59 (m, 1H), 5.35 (dd, J=12.7, 5.3 Hz, 1H), 5.06-5.00 (m, 3H), 4.83-4.81 (m, 2H), 4.52 (s, 2H), 3.60-3.56 (m, 1H), 3.46-3.40 (m, 2H), 3.25-3.23 (m, 2H), 3.16-2.96 (m, 1H), 2.92-2.84 (m, 3H), 2.70-2.64 (m, 4H), 2.60 (s, 2H), 2.47-2.44 (m 1H), 2.22-2.20 (m, 2H), 2.09-1.97 (m, 3H), 1.89-1.80 (m, 5H), 1.71 (s, 2H), 1.62 (s, 2H), 1.41-1.23 (m, 2H), 1.03-1.02 (m, 6H). LC-MS (ESI): m/z [M+H]⁺=935.27.

### Example 178: (1S,3R)-3-(3-((2-(1-((1-(1-(2,6-dioxopiperidin-3-yl)-2-oxo-1,2,3,4-tetrahydro-5-oxa-1,2a-diazaacenaphthylen-6-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

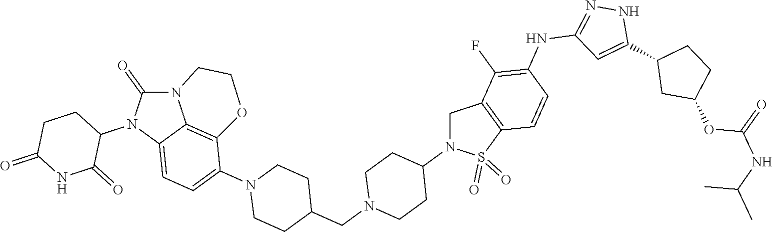 [SMILES: CC(C)NC(=O)O[C@H]1CC[C@@H](c2cc(Nc3ccc4c(c3F)CN(C3CCN(CC5CCN(c6ccc7c8c6OCCn8c(=O)n7C6CCC(=O)NC6=O)CC5)CC3)S4(=O)=O)n[nH]2)C1]

### Step 1: (2-(6-bromo-3-fluoro-2-nitrophenoxy)ethoxy)(tert-butyl)dimethylsilane

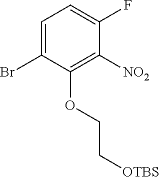 [SMILES: CC(C)(C)[Si](C)(C)OCCOc1c(Br)ccc(F)c1[N+](=O)[O-]]

To a solution of 6-bromo-3-fluoro-2-nitrophenol (7 g, 29.8 mmol) in DMF (100 mL) were added (2-bromoethoxy)(tert-butyl)dimethylsilane (22.68 g, 95.32 mmol) and Cs₂CO₃ (34.2 g, 105 mmol). The resulting solution was stirred overnight at 50° C. under nitrogen atmosphere. The resulting mixture was quenched with water and extracted with EtOAc. The organic layers were washed with brine and concentrated under vacuum. The residue was purified by combi-flash (EtOAc/PE=15%) to give the product (7.5 g, 63.5%) as light yellow oil. MS (ESI) m/e [M+1]⁺=396/394.

### Step 2: 2,6-bis(benzyloxy)-N-(4-bromo-3-(2-((tert-butyldimethylsilyl)oxy)ethoxy)-2-nitrophenyl)pyridin-3-amine

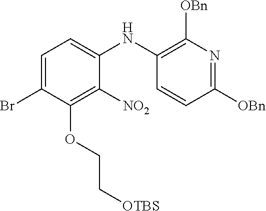 [SMILES: CC(C)(C)[Si](C)(C)OCCOc1c(Br)ccc(Nc2ccc(OCc3ccccc3)nc2OCc2ccccc2)c1[N+](=O)[O-]]

To a solution of 2,6-bis(benzyloxy)pyridin-3-amine (5 g, 16.33 mmol) in THF (300 mL) was added LiHMDS (1 M in THF, 50 mL, 33 mmol) dropwise in 30 min at 0° C., the reaction solution was stirred for 1 h at 0° C., then to this was added (2-(6-bromo-3-fluoro-2-nitrophenoxy)ethoxy)(tert-butyl)dimethylsilane (7.3 g, 19.6 mmol) in THF (50 mL) dropwise in 20 min. The resulting solution was stirred for 3 h at rt. The reaction was quenched by the addition of sat·aq. NH₄Cl solution. The resulting solution was extracted with 3×200 mL of EtOAc. The resulting mixture was concentrated under vacuum. The residue was purified by combi-flash (EA/PE=0%-50%) to give the product (7 g, 55.5%) as red oil. MS (ESI) m/e [M+1]⁺=682/680.

### Step 3: N¹-(2,6-bis(benzyloxy)pyridin-3-yl)-4-bromo-3-(2-((tert-butyldimethylsilyl)oxy)ethoxy)benzene-1,2-diamine

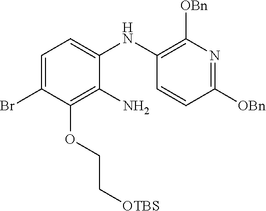 [SMILES: CC(C)(C)[Si](C)(C)OCCOc1c(Br)ccc(Nc2ccc(OCc3ccccc3)nc2OCc2ccccc2)c1N]

To a solution of 2,6-bis(benzyloxy)-N-(4-bromo-3-(2-((tert-butyldimethylsilyl)oxy)ethoxy)-2-nitrophenyl)pyridin-3-amine (7 g, 10.3 mmol) in MeOH:H₂O=4:1 (50 mL) were added Fe (3 g, 51.4 mmol) and NH₄Cl (3 g, 51.4 mmol). The resulting solution was stirred for 3 h at 80° C. The resulting solution was extracted with 3×300 mL of DCM. The resulting mixture was concentrated under vacuum. The residue was purified by combi-flash (EA/PE=0%-50%) to give the product (7 g, 90%) as yellow oil. MS (ESI) m/e [M+1]⁺=652/650.

### Step 4: 1-(2,6-bis(benzyloxy)pyridin-3-yl)-5-bromo-4-(2-((tert-butyldimethylsilyl) oxy)ethoxy)-1,3-dihydro-2H-benzo[d]imidazol-2-one

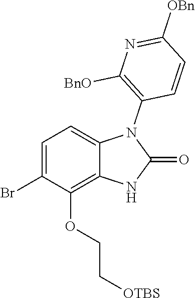 [SMILES: CC(C)(C)[Si](C)(C)OCCOc1c(Br)ccc2c1[nH]c(=O)n2-c1ccc(OCc2ccccc2)nc1OCc1ccccc1]

To a solution of N¹-(2,6-bis(benzyloxy)pyridin-3-yl)-4-bromo-3-(2-((tert-butyldimethylsilyl)oxy)ethoxy)benzene-1,2-diamine (7 g, 10.8 mmol) in ACN (100 mL) was added CDI (2.6 g, 26.9 mmol). The resulting solution was stirred for 16 h at 85° C. The resulting mixture was concentrated under vacuum. The residue was purified by combi-flash (EtOAc/PE=50%) to give the product (6 g, 82.4%) as red solid. MS (ESI) m/e [M+1]⁺=678/676.

### Step 5: 1-(2,6-bis(benzyloxy)pyridin-3-yl)-5-bromo-4-(2-hydroxyethoxy)-1,3-dihydro-2H-benzo[d]imidazol-2-one

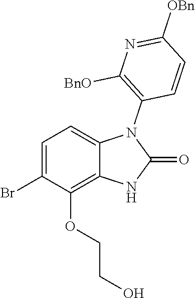 [SMILES: O=c1[nH]c2c(OCCO)c(Br)ccc2n1-c1ccc(OCc2ccccc2)nc1OCc1ccccc1]

To a solution of 1-(2,6-bis(benzyloxy)pyridin-3-yl)-5-bromo-4-(2-((tert-butyldimethylsilyl) oxy)ethoxy)-1,3-dihydro-2H-benzo[d]imidazol-2-one (6 g, 8.88 mmol) in DMF (120 mL) was added CsF (2.7 g, 17.7 mmol). The resulting solution was stirred at room temperature for 4 h under nitrogen atmosphere. The resulting mixture was quenched with water and extracted with EtOAc. The organic layers were washed with brine and concentrated under vacuum to give the product (5.3 g, 97.9%) as purple solid. MS (ESI) m/e [M+1]⁺=562/564.

### Step 6: 1-(2,6-bis(benzyloxy)pyridin-3-yl)-6-bromo-3,4-dihydro-5-oxa-1,2a-diazaacenaphthylen-2(1H)-one

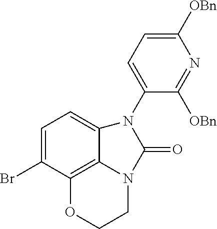 [SMILES: O=c1n2c3c(c(Br)ccc3n1-c1ccc(OCc3ccccc3)nc1OCc1ccccc1)OCC2]

To a solution of 1-(2,6-bis(benzyloxy)pyridin-3-yl)-5-bromo-4-(2-hydroxyethoxy)-1,3-dihydro-2H-benzo[d]imidazol-2-one (4.78 g, 8.52 mmol) in THF (100 mL) were added DIAD (2.58 g, 12.78 mmol), PPh₃ (3.36 g, 12.78 mmol) at 0° C. The resulting solution was stirred for 16 h at rt under N₂ atmosphere. The resulting mixture was concentrated under vacuum. The residue was purified by combi-flash (EtOAc/PE=54:46) to give the product (4 g, 86.5%) as white solid. MS (ESI) m/e [M+1]⁺=544/546.

### Step 7: 1-(2,6-bis(benzyloxy)pyridin-3-yl)-6-(4-(dimethoxymethyl) piperidin-1-yl)-3,4-dihydro-5-oxa-1,2a-diazaacenaphthylen-2(1H)-one

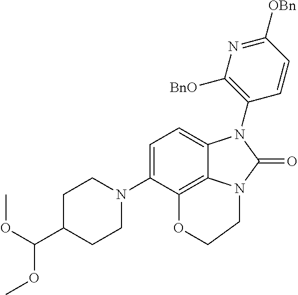 [SMILES: COC(OC)C1CCN(c2ccc3c4c2OCCn4c(=O)n3-c2ccc(OCc3ccccc3)nc2OCc2ccccc2)CC1]

To a solution of 1-(2,6-bis(benzyloxy)pyridin-3-yl)-6-bromo-3,4-dihydro-5-oxa-1,2a-diazaacenaphthylen-2(1H)-one (PH-BGUS-H-A636-6) (1.5 g, 2.8 mmol) in dioxane (20 mL) were added 4-(dimethoxymethyl) piperidine (614.4 mg, 3.9 mmol), Pd-PEPPSI-IPentCl 2-methylpyridine (o-picoline) (115.9 mg, 0.1 mmol) and Cs₂CO₃ (2.7 g, 8.4 mmol). The resulting solution was stirred for 3 h at 100° C. under nitrogen atmosphere. The resulting mixture was concentrated under vacuum. The residue was purified by combi-flash (EtOAc/PE=80%) to give the product (1.4 g, 82.4%) as yellow solid. MS (ESI) m/e [M+1]⁺=623.

### Step 8: 3-(6-(4-(dimethoxymethyl) piperidin-1-yl)-2-oxo-3,4-dihydro-5-oxa-1,2a-diazaacenaphthylen-1(2H)-yl) piperidine-2,6-dione

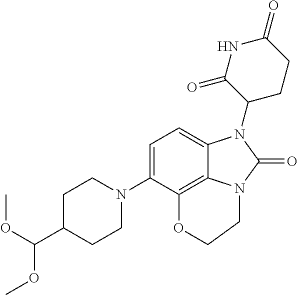 [SMILES: COC(OC)C1CCN(c2ccc3c4c2OCCn4c(=O)n3C2CCC(=O)NC2=O)CC1]

To a solution of 1-(2,6-bis(benzyloxy)pyridin-3-yl)-6-(4-(dimethoxymethyl) piperidin-1-yl)-3,4-dihydro-5-oxa-1,2a-diazaacenaphthylen-2(1H)-one (1.4 g, 2.2 mmol) in THF (20 mL) was added Pd/C (1.4 g, 10% wt). The resulting solution was stirred for 12 h at 50° C. under H₂ atmosphere. After filtration, the filtrate was concentrated under vacuum. The residue was the product (954.1 mg, 97.7%) as white solid. MS (ESI) m/e [M+1]⁺=445.

### Step 9: 1-(1-(2,6-dioxopiperidin-3-yl)-2-oxo-1,2,3,4-tetrahydro-5-oxa-1,2a-diazaacenaphthylen-6-yl)piperidine-4-carbaldehyde

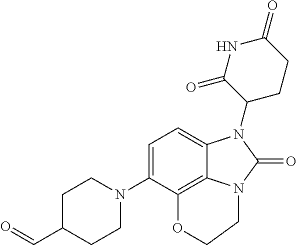 [SMILES: O=CC1CCN(c2ccc3c4c2OCCn4c(=O)n3C2CCC(=O)NC2=O)CC1]

To a flask was added 3-(6-(4-(dimethoxymethyl) piperidin-1-yl)-2-oxo-3,4-dihydro-5-oxa-1,2a-diazaacenaphthylen-1(2H)-yl) piperidine-2,6-dione (200 mg, 0.45 mmol) and formic acid (5 mL). The resulting solution was stirred for 2 h at room temperature. The filtrate was concentrated under vacuum. The residue was the crude product. MS (ESI) m/e [M+1]⁺=399.4

### Step 10: (1S,3R)-3-(3-((2-(1-((1-(1-(2,6-dioxopiperidin-3-yl)-2-oxo-1,2,3,4-tetrahydro-5-oxa-1,2a-diazaacenaphthylen-6-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

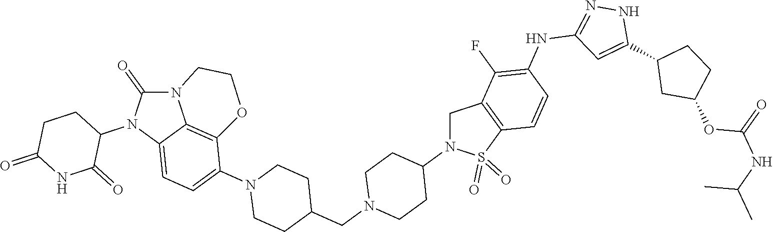 [SMILES: CC(C)NC(=O)O[C@H]1CC[C@@H](c2cc(Nc3ccc4c(c3F)CN(C3CCN(CC5CCN(c6ccc7c8c6OCCn8c(=O)n7C6CCC(=O)NC6=O)CC5)CC3)S4(=O)=O)n[nH]2)C1]

The titled compound was synthesized in the procedures similar to Example 36. LC-MS (ESI): m/z [M+H]⁺=903.45; ¹H NMR (500 MHz, DMSO) δ=12.01 (s, 1H), 11.09 (s, 1H), 8.83 (d, J=21.0, 1H), 8.30 (m, 1H), 7.56 (m, 1H), 6.93 (d, J=7.5, 1H), 6.65 (s, 2H), 5.84 (s, 1H), 5.30 (dd, J=13.0, 5.0, 1H), 5.00 (s, 1H), 4.56 (d, J=6.8, 2H), 4.42 (s, 2H), 3.95 (s, 2H), 3.79 (m, 1H), 3.65 (d, J=10.8, 1H), 3.58 (m, 1H), 3.38 (m, 3H), 3.15 (m, 2H), 3.06 (m, 2H), 2.91 (m, 1H), 2.78 (m, 1H), 2.70-2.58 (m, 4H), 2.45 (m, 1H), 2.36 (m, 1H), 2.19 (m, 3H), 2.11-1.97 (m, 3H), 1.96-1.84 (m, 3H), 1.71 (m, 2H), 1.59 (m, 1H), 1.46 (m, 2H), 1.03 (m, 6H).

### Example 179: (1S,3R)-3-(3-((2-(1′-(1-(2,6-dioxopiperidin-3-yl)-2-oxo-1,2,3,4-tetrahydro-5-oxa-1,2a-diazaacenaphthylen-6-yl)-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

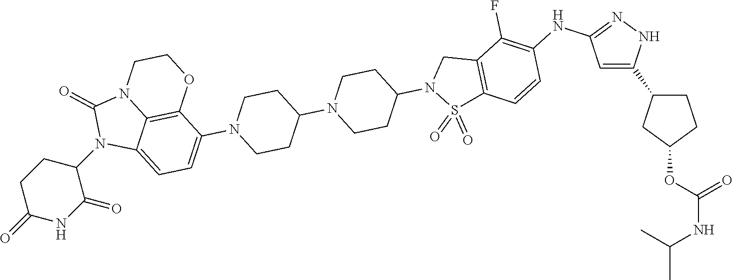 [SMILES: CC(C)NC(=O)O[C@H]1CC[C@@H](c2cc(Nc3ccc4c(c3F)CN(C3CCN(C5CCN(c6ccc7c8c6OCCn8c(=O)n7C6CCC(=O)NC6=O)CC5)CC3)S4(=O)=O)n[nH]2)C1]

### Step 1: 1-(2,6-bis(benzyloxy)pyridin-3-yl)-6-(1,4-dioxa-8-azaspiro [4.5]decan-8-yl)-3,4-dihydro-5-oxa-1,2a-diazaacenaphthylen-2(1H)-one

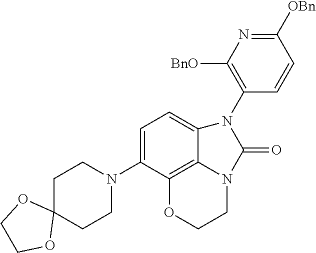 [SMILES: O=c1n2c3c(c(N4CCC5(CC4)OCCO5)ccc3n1-c1ccc(OCc3ccccc3)nc1OCc1ccccc1)OCC2]

To a solution of 1-(2,6-bis(benzyloxy)pyridin-3-yl)-6-bromo-3,4-dihydro-5-oxa-1,2a-diazaacenaphthylen-2(1H)-one (2.5 g, 4.6 mmol) in dioxane (50 mL) were added 1,4-dioxa-8-azaspiro [4.5]decane (921 mg, 6.4 mmol), Pd-PEPPSI-IPentCl 2-methylpyridine (o-picoline) (193.2 mg, 0.2 mmol) and Cs₂CO₃ (4.5 g, 13.8 mmol). The resulting solution was stirred for 3 h at 100° C. under nitrogen atmosphere. The resulting mixture was concentrated under vacuum. The residue was purified by combi-flash (EtOAc/PE=99%) to give the product (2.6 g, 92.9%) as yellow solid. MS (ESI) m/e [M+1]⁺=607.

### Step 2: 3-(2-oxo-6-(1,4-dioxa-8-azaspiro [4.5]decan-8-yl)-3,4-dihydroacenaphthylen-1(2H)-yl) piperidine-2,6-dione

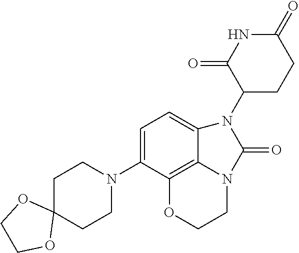 [SMILES: O=C1CCC(n2c(=O)n3c4c(c(N5CCC6(CC5)OCCO6)ccc42)OCC3)C(=O)N1]

To a solution of 1-(2,6-bis(benzyloxy)pyridin-3-yl)-6-(1,4-dioxa-8-azaspiro [4.5]decan-8-yl)-3,4-dihydro-5-oxa-1,2a-diazaacenaphthylen-2(1H)-one (2.5 g, 4.1 mmol) in THF (50 mL) was added Pd/C (2.5 g, 10% wt). The resulting solution was stirred for 12 h at 50° C. under H₂ atmosphere. After filtration, the filtrate was concentrated under vacuum. The residue was the product (1.7 g, 94.4%) as yellow solid. MS (ESI) m/e [M+1]⁺=429.

### Step 3: 3-(2-oxo-6-(4-oxopiperidin-1-yl)-3,4-dihydro-5-oxa-1,2a-diazaacenaphthylen-1(2H)-yl) piperidine-2,6-dione

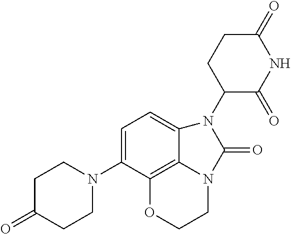 [SMILES: O=C1CCN(c2ccc3c4c2OCCn4c(=O)n3C2CCC(=O)NC2=O)CC1]

The solution of 3-(2-oxo-6-(1,4-dioxa-8-azaspiro [4.5]decan-8-yl)-3,4-dihydroacenaphthylen-1(2H)-yl) piperidine-2,6-dione (1.7 g, 4 mmol) in AcOH/H₂O=4:1 (20 mL) was stirred for 10 h at 80° C. The resulting mixture was concentrated under vacuum. The residue was purified by combi-flash (MeOH/DCM=9%) to give the product (1.1 g, 73.3%) as yellow green solid. MS (ESI) m/e [M+1]⁺=385.

### Step 4: (1S,3R)-3-(3-((2-(1′-(1-(2,6-dioxopiperidin-3-yl)-2-oxo-1,2,3,4-tetrahydro-5-oxa-1,2a-diazaacenaphthylen-6-yl)-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

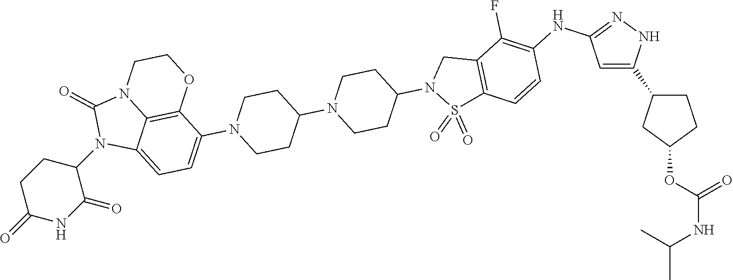 [SMILES: CC(C)NC(=O)O[C@H]1CC[C@@H](c2cc(Nc3ccc4c(c3F)CN(C3CCN(C5CCN(c6ccc7c8c6OCCn8c(=O)n7C6CCC(=O)NC6=O)CC5)CC3)S4(=O)=O)n[nH]2)C1]

The titled compound was synthesized in the procedures similar to Example 39. LC-MS (ESI): m/z [M+H]⁺=889.61; ¹H NMR (500 MHz, DMSO) δ=12.02 (s, 1H), 11.08 (s, 1H), 8.83 (d, J=23.2, 1H), 8.29 (s, 1H), 7.59-7.53 (m, 1H), 6.94 (d, J=7.9, 1H), 6.62 (t, J=7.2, 1H), 6.58-6.51 (m, 1H), 5.84 (s, 1H), 5.29 (dd, J=13.0, 5.2, 1H), 5.00 (s, 1H), 4.58-4.53 (m, 2H), 4.40 (s, 2H), 3.93 (m, 2H), 3.85 (m, 2H), 3.76 (m, 1H), 3.61 (m, 2H), 3.52 (m, 3H), 3.36 (m, 1H), 3.24 (m, 1H), 3.08 (m, 1H), 2.92 (m, 1H), 2.66-2.62 (m, 3H), 2.60 (m, 1H), 2.45 (m, 1H), 2.25 (m, 1H), 2.18-2.05 (m, 4H), 2.04-1.97 (m, 2H), 1.91 (m, 1H), 1.89-1.82 (m, 1H), 1.72 (m, 3H), 1.59 (m, 1H), 1.03 (m, 6H).

### Example 180: (1S,3R)-3-(3-((2-(1-((1-(1-(2,6-dioxopiperidin-3-yl)-2-oxo-1,2,5,6-tetrahydro-4H-imidazo[4,5,1-ij]quinolin-7-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

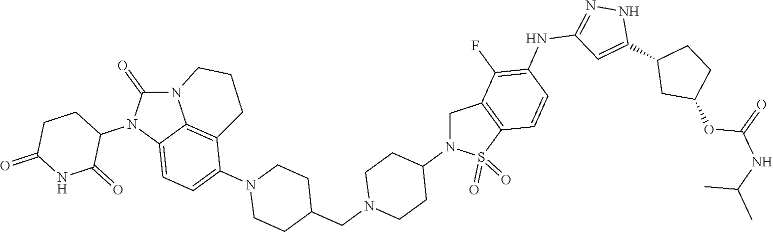 [SMILES: CC(C)NC(=O)O[C@H]1CC[C@@H](c2cc(Nc3ccc4c(c3F)CN(C3CCN(CC5CCN(c6ccc7c8c6CCCn8c(=O)n7C6CCC(=O)NC6=O)CC5)CC3)S4(=O)=O)n[nH]2)C1]

The titled compound was synthesized in the procedures similar to Example 36. LC-MS (ESI): m/z [M+H]⁺=901.68; ¹H NMR (500 MHz, DMSO) δ=12.03 (s, 1H), 11.07 (s, 1H), 8.83 (d, J=21.9, 1H), 8.30 (m, 1H), 7.56 (m7, 1H), 6.93 (d, J=7.5, 1H), 6.86 (d, J=7.8, 1H), 6.66 (s, 1H), 5.84 (s, 1H), 5.29 (dd, J=13.0, 5.1, 1H), 5.00 (s, 1H), 4.56 (d, J=6.7, 2H), 3.80 (m, 1H), 3.74 (m, 2H), 3.65 (m, 2H), 3.61-3.56 (m, 1H), 3.48 (m, 1H), 3.30 (m, 1H), 3.16 (m, 4H), 3.07 (m, 2H), 2.92 (m, 1H), 2.75 (m, 2H), 2.69-2.61 (m, 4H), 2.45 (m, 1H), 2.36 (m, 1H), 2.20 (m, 2H), 2.08-1.95 (m, 5H), 1.94-1.84 (m, 3H), 1.72 (m, 2H), 1.59 (m, 1H), 1.44 (m, 2H), 1.03 (m, 6H).

### Example 181: (1S,3R)-3-(3-((2-(1′-(1-(2,6-dioxopiperidin-3-yl)-2-oxo-1,2,5,6-tetrahydro-4H-imidazo[4,5,1-ij]quinolin-7-yl)-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

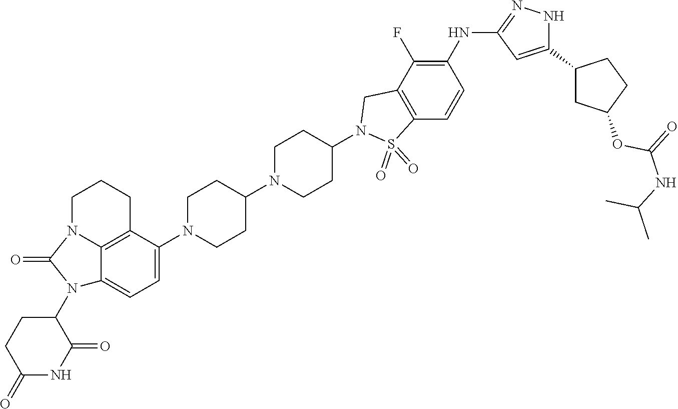 [SMILES: CC(C)NC(=O)O[C@H]1CC[C@@H](c2cc(Nc3ccc4c(c3F)CN(C3CCN(C5CCN(c6ccc7c8c6CCCn8c(=O)n7C6CCC(=O)NC6=O)CC5)CC3)S4(=O)=O)n[nH]2)C1]

### Step 1: 5-bromo-1,2,3,4-tetrahydroquinolin-8-amine5-bromo-1,2,3,4-tetrahydroquinolin-8-amine

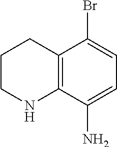 [SMILES: Nc1ccc(Br)c2c1NCCC2]

To a solution of 5-bromoquinolin-8-amine (25.0 g, 112.1 mmol, 1.0 eq) in AcOH (250 mL) was added PtO₂ (2.5 g, 11.2 mmol, 0.1 eq) under H₂, the mixture was stirred at room temperature for 48 h under H₂. After completion, the mixture was filtered and the filter cake was washed with MeOH (50 mL). The filtrate was concentrated to give a residue, which was purified by silica gel column chromatography (Petroleum ether/EtOAc=1:1) to give a crude product. Then the crude product was purified by reversed-phase chromatography (acetonitrile in water, 30% to 35%) to give 5-bromo-1,2,3,4-tetrahydroquinolin-8-amine5-bromo-1,2,3,4-tetrahydroquinolin-8-amine (6.6 g, crude) as a yellow solid.

### Step 2: 7-bromo-5,6-dihydro-4H-imidazo[4,5,1-ij]quinolin-2(1H)-one

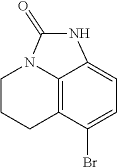 [SMILES: O=c1[nH]c2ccc(Br)c3c2n1CCC3]

To a solution of 5-bromo-1,2,3,4-tetrahydroquinolin-8-amine5-bromo-1,2,3,4-tetrahydroquinolin-8-amine (7.3 g, 32.1 mmol, 1.0 eq) in THF (365 mL) was added TEA (9.7 g, 96.3 mmol, 3.0 eq) and Triphosgene (6.7 g, 22.5 mmol, 0.7 eq) at 0° C. under N₂, the mixture was stirred at 0° C. for 1 h. After completion, the mixture was quenched with saturated NH₄Cl aqueous (80 mL) and extracted with EtOAc (80 mL×3), the combined organic phase was washed with brine (80 mL×3), dried over Na₂SO₄ and concentrated to give a residue, which was purified by silica gel column chromatography (Petroleum ether/EtOAc=1:1) to give 7-bromo-5,6-dihydro-4H-imidazo[4,5,1-ij]quinolin-2(1H)-one (4.6 g, 56.5%) as a yellow solid.

### Step 3: 1-(4-methoxybenzyl)-2,6-dioxopiperidin-3-yl trifluoromethanesulfonate

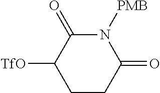

To a solution of 3-hydroxy-1-(4-methoxybenzyl)piperidine-2,6-dione (20.0 g, 80.2 mmol, 1.0 eq) and Pyridine (12.7 g, 160.5 mmol, 2.0 eq) in DCM (200 mL) was added Tf₂O (33.9 g, 120.3 mmol, 1.5 eq) dropwise at 0° C., the mixture was stirred at −10° C. for 1.5 h under N₂. After completion, the mixture was quenched with saturated NaHCO₃ aqueous (100 mL) and extracted with EtOAc (100 mL×2), the combined organic phase was washed with brine (100 mL×2), dried over Na₂SO₄ and concentrated to give a residue, which was purified by silica gel column chromatography (Petroleum ether/EtOAc=5:1) to give 1-(4-methoxybenzyl)-2,6-dioxopiperidin-3-yl trifluoromethanesulfonate (19.0 g, 62.1%) as a white solid.

### Step 4: 3-(7-bromo-2-oxo-5,6-dihydro-4H-imidazo[4,5,1-ij]quinolin-1(2H)-yl)-1-(4-methoxybenzyl)piperidine-2,6-dione

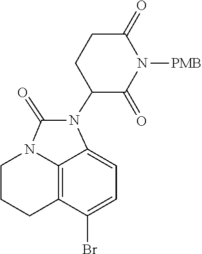

To a solution of 7-bromo-5,6-dihydro-4H-imidazo[4,5,1-ij]quinolin-2(1H)-on (4.0 g, 15.8 mmol, 1.0 eq) in THF (40 mL) was added tBuOK (2.7 g, 23.7 mmol, 1.5 eq) at 0° C. under N₂, the mixture was stirred at 0° C. for 1 h, then added the solution of 1-(4-methoxybenzyl)-2,6-dioxopiperidin-3-yl trifluoromethanesulfonate (9.2 g, 24.1 mmol, 1.5 eq) in THF (40 mL) dropwise at 0° C. and stirred at 0-10° C. for 1 h. After completion, the mixture was quenched with water (200 mL) and extracted with EtOAc (200 mL×3), the combined organic phase was washed with brine (200 mL×3), dried over Na₂SO₄ and concentrated to give 3-(7-bromo-2-oxo-5,6-dihydro-4H-imidazo[4,5,1-ij]quinolin-1(2H)-yl)-1-(4-methoxybenzyl)piperidine-2,6-dione (5.4 g, 70.5%) as a yellow solid.

### Step 5: 3-(7-bromo-2-oxo-5,6-dihydro-4H-imidazo[4,5,1-ij]quinolin-1(2H)-yl)piperidine-2,6-dione

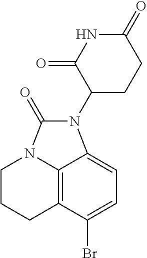 [SMILES: O=C1CCC(n2c(=O)n3c4c(c(Br)ccc42)CCC3)C(=O)N1]

To a solution of 3-(7-bromo-2-oxo-5,6-dihydro-4H-imidazo[4,5,1-ij]quinolin-1(2H)-yl)-1-(4-methoxybenzyl)piperidine-2,6-dione (5.8 g, 12.0 mmol, 1.0 eq) in Toluene (58 mL) was added CH₃HSO₃ (21.9, 228.0 mmol, 19.0 eq) under N₂, the mixture was stirred at 120° C. for 2 h under N₂. After completion, the mixture was cooled, poured into ice-water (290 mL) and extracted with EtOAc (580 mL×3), the combined organic phase was washed with brine (290 mL×3), dried over Na₂SO₄ and concentrated to give a residue, which was triturated by EtOAc (90 mL) to give 3-(7-bromo-2-oxo-5,6-dihydro-4H-imidazo[4,5,1-ij]quinolin-1(2H)-yl)piperidine-2,6-dione (3.0 g, 68.7%) as a white solid.

### Step 6: 3-(2-oxo-7-(1,4-dioxa-8-azaspiro[4.5]decan-8-yl)-5,6-dihydro-4H-imidazo[4,5,1-ij]quinolin-1(2H)-yl)piperidine-2,6-dione

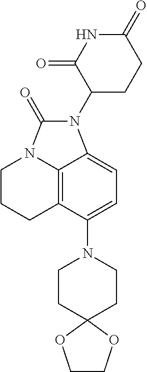 [SMILES: O=C1CCC(n2c(=O)n3c4c(c(N5CCC6(CC5)OCCO6)ccc42)CCC3)C(=O)N1]

To a solution of 3-(7-bromo-2-oxo-5,6-dihydro-4H-imidazo[4,5,1-ij]quinolin-1(2H)-yl)piperidine-2,6-dione (1.2 g, 3.3 mmol, 1.0 eq) and 1,4-dioxa-8-azaspiro[4.5]decane (945 mg, 6.6 mmol, 2.0 eq) in 1,4-dioxane (130 mL) was added Cs₂CO₃ (1.6 g, 5.0 mmol, 1.5 eq) and [1,3-bis[2,6-bis(1-ethylpropyl)phenyl]-4,5-dichloro-1,3-dihydro-2H-imidazol-2-ylidene]dichloro(2-methylpyridine)-, (SP-4-1)-(ACI) (277 mg, 0.33 mmol, 0.1 eq) under N₂, the mixture was stirred at 100° C. for 5 h under N₂. After completion, the mixture was cooled and filtered. The filter cake was washed with DCM (100 mL) and the filtrate was concentrated to give a residue, which was purified by silica gel column chromatography (EtOAc) give 3-(2-oxo-7-(1,4-dioxa-8-azaspiro[4.5]decan-8-yl)-5,6-dihydro-4H-imidazo[4,5,1-ij]quinolin-1(2H)-yl)piperidine-2,6-dione (400 mg, 28.5%) as a white solid.

### Step 7: 3-(2-oxo-7-(4-oxopiperidin-1-yl)-5,6-dihydro-4H-imidazo[4,5,1-ij]quinolin-1(2H)-yl)piperidine-2,6-dione

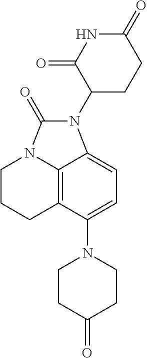 [SMILES: O=C1CCN(c2ccc3c4c2CCCn4c(=O)n3C2CCC(=O)NC2=O)CC1]

To a solution of 3-(2-oxo-7-(1,4-dioxa-8-azaspiro[4.5]decan-8-yl)-5,6-dihydro-4H-imidazo[4,5,1-ij]quinolin-1(2H)-yl)piperidine-2,6-dione (1.2 g, 2.8 mmol, 1.0 eq) in 12 N HCl aqueous (15 mL), the mixture was stirred at room temperature for 24 h. After completion, the mixture was adjusted pH to 6-7 with sat. aq. NaHCO: solution and extracted with DCM (50 mL×3), the combined organic phase was dried over Na₂SO₄ and concentrated to give a residue. The residue was purified by silica gel column chromatography (EtOAc) to give 3-(2-oxo-7-(4-oxopiperidin-1-yl)-5,6-dihydro-4H-imidazo[4,5,1-ij]quinolin-1(2H)-yl)piperidine-2,6-dione (610 mg, 56.7%) as a yellow solid.

### Step 8: (1S,3R)-3-(3-((2-(1′-(1-(2,6-dioxopiperidin-3-yl)-2-oxo-1,2,5,6-tetrahydro-4H-imidazo[4,5,1-ij]quinolin-7-yl)-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

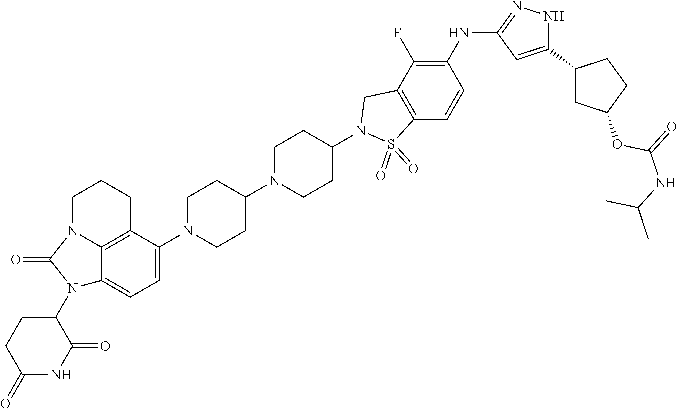 [SMILES: CC(C)NC(=O)O[C@H]1CC[C@@H](c2cc(Nc3ccc4c(c3F)CN(C3CCN(C5CCN(c6ccc7c8c6CCCn8c(=O)n7C6CCC(=O)NC6=O)CC5)CC3)S4(=O)=O)n[nH]2)C1]

The titled compound was synthesized in the procedures similar to Example 39. ¹H NMR (500 MHz, DMSO-d₆) δ 12.00 (s, 1H), 11.05 (s, 1H), 8.76 (s, 1H), 8.29 (s, 1H), 7.51 (d, J=10 Hz, 1H), 6.98-6.91 (m, 1H), 6.81-6.75 (m, 1H), 6.61-6.58 (m, 1H), 5.83 (s, 1H), 5.33-5.24 (m, 1H), 5.06-4.96 (m, 1H), 4.52 (s, 2H), 3.65-3.52 (m, 2H), 3.48-3.45 (m, 2H), 3.21-3.12 (m, 2H), 3.08-2.72 (m, 5H), 2.61-2.52 (m, 7H), 2.47-2.45 (m, 1H), 2.17-1.77 (m, 12H), 1.71-1.52 (m, 5H), 1.15-0.99 (m, 6H). LC-MS (ESI): m/z [M+H]⁺=887.

### Example 182: (1S,3R)-3-(3-((2-(1′-(1-((S)-2,6-dioxopiperidin-3-yl)-4,7-difluoro-3-methyl-2-oxo-2,3-dihydro-1H-benzo[d]imidazol-5-yl)-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

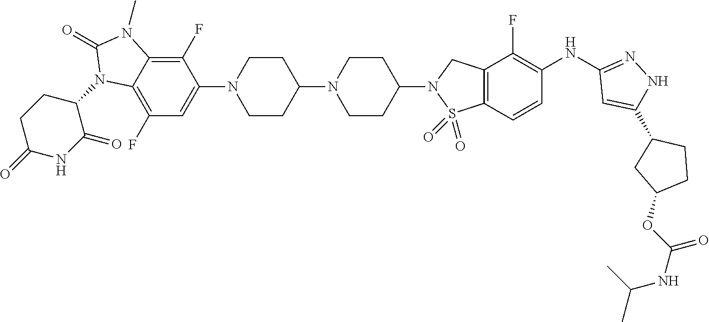 [SMILES: CC(C)NC(=O)O[C@H]1CC[C@@H](c2cc(Nc3ccc4c(c3F)CN(C3CCN(C5CCN(c6cc(F)c7c(c6F)n(C)c(=O)n7[C@H]6CCC(=O)NC6=O)CC5)CC3)S4(=O)=O)n[nH]2)C1]

### Step 1: 3-(4,7-difluoro-3-methyl-2-oxo-5-(1,4-dioxa-8-azaspiro[4.5]decan-8-yl)-2,3-dihydro-1H-benzo[d]imidazol-1-yl)piperidine-2,6-dione

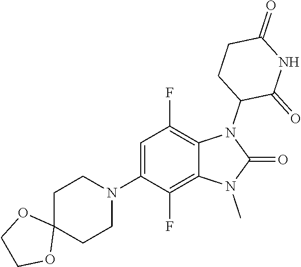 [SMILES: Cn1c(=O)n(C2CCC(=O)NC2=O)c2c(F)cc(N3CCC4(CC3)OCCO4)c(F)c21]

To a solution of 3-(5-bromo-4,7-difluoro-3-methyl-2-oxo-2,3-dihydro-1H-benzo[d]imidazol-1-yl)piperidine-2,6-dione (PH-BGUS-H-A601-21) (3 g, 8 mmol) in dioxane (60 mL) were added 1-(piperidin-4-yl)cyclopropan-1-ol (1.7 g, 12 mmol), Pd-PEPPSI-IPentCl 2-methylpyridine (336 mg, 0.4 mmol) and Cs₂CO₃ (3.9 g, 12 mmol). The resulting solution was stirred for 12 h at 100° C. under N₂ atmosphere. The resulting mixture was concentrated under vacuum. The residue was purified by combi-flash (EtOAc/PE=43%) to give the product (1.6 g, 45%) as yellow solid. LCMS (M+H) m/z: 437.

### Step 2: 3-(4,7-difluoro-3-methyl-2-oxo-5-(4-oxopiperidin-1-yl)-2,3-dihydro-1H-benzo[d]imidazol-1-yl)piperidine-2,6-dione

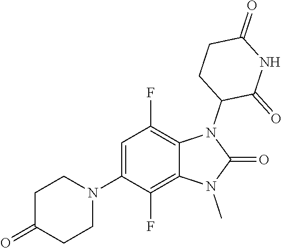 [SMILES: Cn1c(=O)n(C2CCC(=O)NC2=O)c2c(F)cc(N3CCC(=O)CC3)c(F)c21]

To a solution of 3-(4,7-difluoro-3-methyl-2-oxo-5-(1,4-dioxa-8-azaspiro[4.5]decan-8-yl)-2,3-dihydro-1H-benzo[d]imidazol-1-yl)piperidine-2,6-dione (550 mg, 4.8 mmol) in H2SO4 (20 mL) at 0° C. The resulting solution was stirred for 3 h at rt. The mixture was basified to pH=7 with Na2CO3. The resulting mixture was extracted with EA. The organic layers were washed with brine, dried over anhydrous Na₂SO₄ The reaction was concentrated under reduced pressure. The residue was the product (828.2 mg, 59%) as white solid. LCMS (M+H) m/z: 393.05.

### Step 3: (1S,3R)-3-(3-((2-(1′-(1-((S)-2,6-dioxopiperidin-3-yl)-4,7-difluoro-3-methyl-2-oxo-2,3-dihydro-1H-benzo[d]imidazol-5-yl)-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

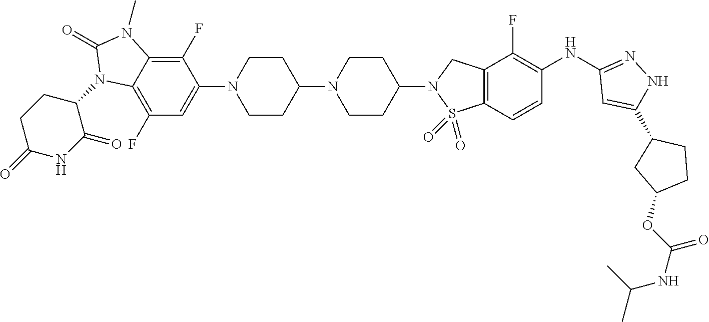 [SMILES: CC(C)NC(=O)O[C@H]1CC[C@@H](c2cc(Nc3ccc4c(c3F)CN(C3CCN(C5CCN(c6cc(F)c7c(c6F)n(C)c(=O)n7[C@H]6CCC(=O)NC6=O)CC5)CC3)S4(=O)=O)n[nH]2)C1]

The titled compound was synthesized in the procedures similar to Example 39 in a racemic form, which was further separated by Chiral Prep-HPLC to give Example 182 (100% ee); Retention time: 9.94 min. ¹H NMR (500 MHz, DMSO) δ 11.99 (s, 1H), 11.11 (s, 1H), 8.76 (s, 1H), 8.29 (s, 1H), 7.51 (d, J=8.6 Hz, 1H), 6.94 (d, J=7.7 Hz, 1H), 6.66 (s, 1H), 5.83 (s, 1H), 5.45 (s, 1H), 5.00 (s, 1H), 4.51 (s, 2H), 3.60-3.55 (m, 1H), 3.50 (s, 3H), 3.40 (s, 3H), 3.08-2.98 (m, 2H), 2.67-2.60 (m, 6H), 2.36 (s, 2H), 2.30 (s, 2H), 2.18 (s, 1H), 2.10 (s, 1H), 2.05-1.98 (m, 2H), 1.95-1.89 (m, 2H), 1.84 (s, 2H), 1.71 (s, 2H), 1.63-1.60 (m, 2H), 1.24 (s, 2H), 1.04-1.02 (m, 6H). LC-MS (ESI): m/z [M+H]⁺=897.33.

Chiral analytical method: Column: YMC Amylose-SA 4.6*250 mm 5 μm; Mobile phase: A for MtBE (0.1%2M NH3 MeOH)(%) and B for DCM:MeOH=50:50(%); Gradient: Mobile Phase A: Mobile Phase B=30:70 (v/v); HPLC Equipment: HPLC-Agilent, Back pressure: 100 bar; Column temperature: 35° C.

Chiral Prep-HPLC Condition: Column: YMC Amylose-SA 4.6*250 mm 5 μm; Mobile phase: A MtBE (0.1%2M NH3 MeOH)(%) and B for DCM:MeOH=50:50(%); Gradient: Mobile Phase A: Mobile Phase B=30:70 (v/v); Flow Rate: 20 mL/min, Wave Length: UV 200 nm and 270 nm, Prep-HPLC Equipment: Prep-HPLC-Gilson, Back pressure: 100 bar; Column temperature: 25° C.

### Example 183: (1S,3R)-3-(3-((2-(1-((1-(1-((S)-2,6-dioxopiperidin-3-yl)-7-fluoro-3,4-dimethyl-2-oxo-2,3-dihydro-1H-benzo[d]imidazol-5-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropyl carbamate

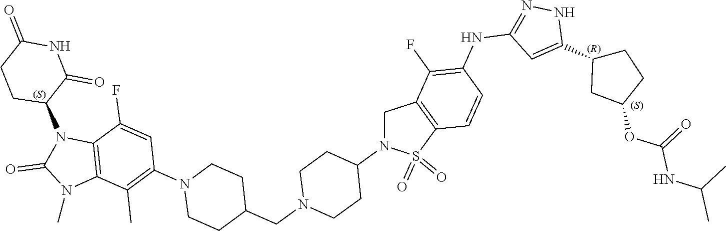

### Step 11: (E)-N-(3-bromo-5-fluoro-2-methylphenyl)-2-(hydroxyimino)acetamide

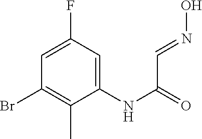

To a solution of sodium sulfate (167.07 g, 1181.6 mmol), hydroxylamine hydrochloride (35.76 g, 517.12 mmol), and chloral (32.5 g, 221.55 mmol) was dissolved in water (750 mL). To the reaction mixture was added a solution of 3-bromo-5-fluoro-2-methylaniline (30 g, 147.7 mmol) in a mixture of water (810 mL), ethanol (105 mL), and concentrated HCl (25.8 mL). The reaction mixture was stirred overnight at 60° C., then cooled to room temperature. A precipitate formed that was collected by filtration and washed with water (2×50 mL). The resulting solid was dried in an oven under reduced pressure to afford the product (30 g, 75%) as yellow solid. LCMS (ES, m/z): 275[M+H]+

### Step 2: 6-bromo-4-fluoro-7-methylindoline-2,3-dione

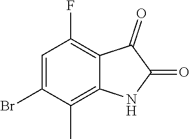

To a solution of (2E)-N-(3-bromo-5-fluoro-2-methylphenyl)-2-(N-hydroxyimino)acetamide (30 g, 109 mmol) was added to sulfuric acid (231 mL) in portions at 60° C. The resulting mixture was stirred for 1 h at 60° C., then cooled to room temperature and slowly added to ice water. A precipitate formed that was collected by filtration and washed with water (2×20 mL). The resulting solid was dried under vacuum. The residue was purified by combi-flash (EtOAc/PE=0:100-50:50) to give the product (13.8 g, 50%) as yellow solid. LCMS (ES, m/z): 258[M+H]⁺

### Step 3: 6-bromo-4-fluoro-1,7-dimethylindoline-2,3-dione

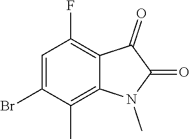

To a solution of 6-bromo-4-fluoro-7-methylindoline-2,3-dione (13.8 g, 53 mmol) in DMF (150 mL) was added sodium hydride (60%)(2.57 g, 63.6 mmol) at 0° C. for 5 min, Then added CH₃I (8.27 g, 58.3 mmol). The resulting solution was stirred for 2 h at rt. The reaction was extracted with EA. The organic layers were washed with brine. dried over anhydrous Na₂SO₄ The reaction was concentrated under reduced pressure. The residue was purified by combi-flash (EtOAc/PE=0:100-50:50) to give the product (11 g, 75%) as white solid. LCMS (ES, m/z): 272[M+H]⁺

### Step 4: 4-bromo-6-fluoro-3-methyl-2-(methylamino)benzoic acid

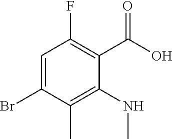

To a solution of 6-bromo-4-fluoro-1,7-dimethylindoline-2,3-dione (11 g, 40.7 mmol) and NaOH (2 M) (54 mL, 382 mmol) was added H₂O₂ (9.1 mL, 211.8 mmol) dropwise over 15 min at room temperature. The resulting mixture was stirred for an additional 3 h at room temperature, then quenched with saturated sodium sulfite (aq.) at room temperature and neutralized to pH 7 with HCl (2 M). The resulting mixture was filtered, and the filter cake washed with water (2×20 mL). The filtrate was concentrated under reduced pressure to give a residue. The residue was acidified to pH 4 with HCl (2 M). The precipitated solids were collected by filtration and washed with water (2×20 mL). The resulting solid was dried under infrared light to afford the product (8 g, 75%) as yellow solid. LCMS (ES, m/z): 262[M+H]+.

### Step 5: 6-bromo-4-fluoro-1,7-dimethyl-1,3-dihydro-2H-benzo[d]imidazol-2-one

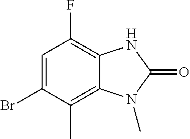

To a solution of 5-bromo-3,6-difluoro-N1-methylbenzene-1,2-diamine (8 g, 30 mmol) in the t-BuOH (80 mL) were added DIEA (10.8 g, 90 mmol) and DPPA (12.3 g, 45 mmol). The mixture was stirred at 95° C. for 12 hrs under N₂. On completion, the mixture was concentrated in vacuo. The residue was diluted with toluene (50 mL), filtered and the filtered cake was collected and dried in vacuo to give the product (4 g, 51%) as gray solid. LCMS (ES, m/z): 259[M+H]+.

### Step 6: 3-(5-bromo-7-fluoro-3,4-dimethyl-2-oxo-2,3-dihydro-1H-benzo[d]imidazol-1-yl)piperidine-2,6-dione

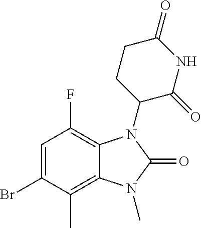 [SMILES: Cc1c(Br)cc(F)c2c1n(C)c(=O)n2C1CCC(=O)NC1=O]

To a solution of 6-bromo-4-fluoro-1,7-dimethyl-1,3-dihydro-2H-benzo[d]imidazol-2-one (4 g, 15.5 mmol) in THF (80 mL) were added sodium hydride (60%)(1.86 g, 46.5 mmol) and 3-bromopiperidine-2,6-dione (2.79 g, 30.4 mmol). The resulting solution was stirred for 12 h at 60° C. The resulting mixture was concentrated under vacuum. The residue was purified by PREP-ACHIRAL-SFC (Column: GreenSep Naphthyl 3*25 cm, 5 μm; Mobile Phase A: CO2, Mobile Phase B: MEOH:DMSO=9:1; Flow rate: 75 mL/min; Gradient (B %): isocratic 18% B; Column Temperature (° C.): 35; Back Pressure (bar): 100; Wave Length: 254 nm; RT1 (min): 4.32; RT2 (min): 6.48; Sample Solvent: ACN; Injection Volume: 3.75 mL; Number Of Runs: 8.0) to give the product (800 mg, 14%) as white solid. LCMS (ES, m/z): 370[M+H]+.

### Step 5: 3-(5-(4-(dimethoxymethyl)piperidin-1-yl)-7-fluoro-3,4-dimethyl-2-oxo-2,3-dihydro-1H-benzo[d]imidazol-1-yl)piperidine-2,6-dione

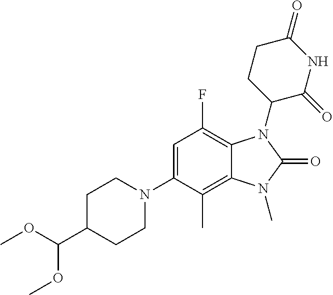 [SMILES: COC(OC)C1CCN(c2cc(F)c3c(c2C)n(C)c(=O)n3C2CCC(=O)NC2=O)CC1]

To a solution of 3-(5-bromo-7-fluoro-3,4-dimethyl-2-oxo-2,3-dihydro-1H-benzo[d]imidazol-1-yl)piperidine-2,6-dione (PH-BGUS-H-A659-5) (300 mg, 0.81 mmol) in DMF (20 mL) were added 4-(dimethoxymethyl)piperidine (191 mg, 1.2 mmol), Cs₂CO₃ (400 g, 1.2 mmol) and Pd-PEPPSI2Me-IPENT Cl (35 mg, 0.04 mmol) for 12 h at 100° C. The mixture was extracted with ethyl acetate (3×30 mL). The organic layers were washed with brine (60 mL), dried over anhydrous Na₂SO₄, filtered and concentrated under reduced pressure to give a residue. The crude was purified by combi-flash (SiO₂, PE/EA=90:10-0:100) to give the product (172.9 mg, 40%) as yellow green solid. LCMS (M+H) m/z: 449.15.

### Step 6: 1-(1-(2,6-dioxopiperidin-3-yl)-7-fluoro-3,4-dimethyl-2-oxo-2,3-dihydro-1H-benzo[d]imidazol-5-yl)piperidine-4-carbaldehyde

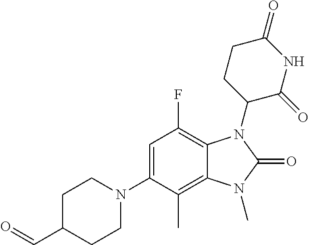 [SMILES: Cc1c(N2CCC(C=O)CC2)cc(F)c2c1n(C)c(=O)n2C1CCC(=O)NC1=O]

To a flask was added 3-(5-(4-(dimethoxymethyl)piperidin-1-yl)-7-fluoro-3,4-dimethyl-2-oxo-2,3-dihydro-1H-benzo[d]imidazol-1-yl)piperidine-2,6-dione (200 mg, 0.45 mmol) and formic acid (5 mL). The resulting solution was stirred for 2 h at room temperature. The filtrate was concentrated under vacuum. The residue was the crude product. MS (ESI) m/e [M+1]⁺=449.4

### Step 7: (1S,3R)-3-(3-((2-(1-((1-(1-((S)-2,6-dioxopiperidin-3-yl)-7-fluoro-3,4-dimethyl-2-oxo-2,3-dihydro-1H-benzo[d]imidazol-5-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropyl carbamate

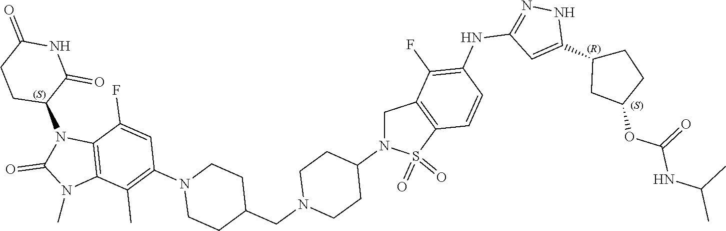

The titled compound was synthesized in the procedures similar to Example 36 in a racemic form, which was further separated by Chiral Prep-HPLC to give Example (100% ee); Retention time: 10.387 min. ¹H NMR (500 MHz, DMSO) δ 11.99 (s, 1H), 11.07 (s, 1H), 8.76 (s, 1H), 8.30 (t, J=8.0 Hz, 1H), 7.51 (d, J=8.7 Hz, 1H), 6.94 (d, J=7.4 Hz, 1H), 6.66 (d, J=13.4 Hz, 1H), 5.83 (s, 1H), 5.47 (d, J=7.6 Hz, 1H), 5.00 (s, 1H), 4.51 (s, 2H), 3.67-3.51 (m, 4H), 3.46-3.40 (m, 1H), 3.09-2.88 (m, 6H), 2.68-2.54 (m, 6H), 2.34-2.10 (m, 3H), 2.10-2.00 (m, 4H), 1.96-1.53 (m, 12H), 1.35-1.24 (m, 2H), 1.08-0.96 (m, 6H). LC-MS (ESI): m/z [M+H]⁺=907.50.

Chiral analytical method: Column: i-Cellulose-5 4.6*250 mm 5 μm Mobile phase: A for MtBE (0.1%2M NH3 MeOH)(%) and B for MeOH:DCM=50:50 (v/v); Gradient: Mobile Phase A: Mobile Phase B=30:70 (v/v); HPLC Equipment: Agilent 1260 Infinity II (ARD_LC-808); Column temperature: 25° C.

Chiral Prep-HPLC Condition: i-Cellulose-5 4.6*250 mm 5 μm Mobile phase: A for MtBE (0.1%2M NH3 MeOH)(%) and B for MeOH:DCM=50:50; Gradient: Mobile Phase A: Mobile Phase B=30:70 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 184: (1S,3R)-3-(3-((2-(1-((1-(1-(2,6-dioxopiperidin-3-yl)-4-methoxy-3-methyl-2-oxo-2,3-dihydro-1H-benzo[d]imidazol-5-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

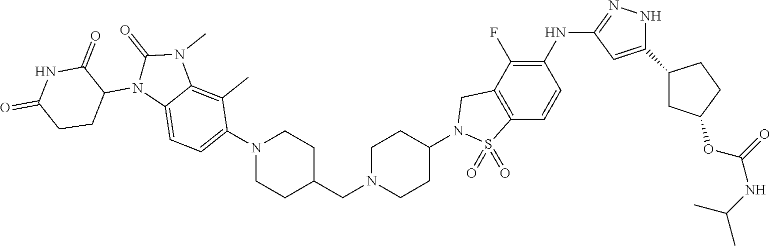 [SMILES: Cc1c(N2CCC(CN3CCC(N4Cc5c(ccc(Nc6cc([C@@H]7CC[C@H](OC(=O)NC(C)C)C7)[nH]n6)c5F)S4(=O)=O)CC3)CC2)ccc2c1n(C)c(=O)n2C1CCC(=O)NC1=O]

The titled compound was synthesized in the procedures similar to Example 185. ¹H NMR (500 MHz, DMSO) δ 11.08 (s, 1H), 9.3-9.13 (m, 1H), 8.86-8.62 (m, 1H), 8.34-8.28 (m, 1H), 7.58-7.54 (m, 1H), 6.94 (d, J=7.4 Hz, 1H), 6.79 (d, J=8.5 Hz, 1H), 6.71 (d, J=8.5 Hz, 1H), 5.84 (s, 1H), 5.33-5.29 (m, 1H), 5.00 (s, 1H), 4.56 (s, 2H), 3.92 (s, 3H), 3.83-3.77 (m, 1H), 3.68-3.57 (m, 2H), 3.50 (s, 3H), 3.38-3.27 (m, 3H), 3.17-3.06 (m, 3H), 2.94-2.86 (m, 1H), 2.70-2.60 (m, 4H), 2.48-2.45 (m, 1H), 2.40-2.35 (m, 1H), 2.23-2.18 (m, 3H), 2.10-1.86 (m, 8H), 1.80-1.68 (m, 2H), 1.62-1.57 (m, 1H), 1.49-1.41 (m, 2H), 1.03 (d, J=6 Hz, 6H). LC-MS (ESI): m/z [M+H]⁺=905.50.

### Example 185: (1S,3R)-3-(3-((2-(1′-(1-(2,6-dioxopiperidin-3-yl)-4-methoxy-3-methyl-2-oxo-2,3-dihydro-1H-benzo[d]imidazol-5-yl)-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

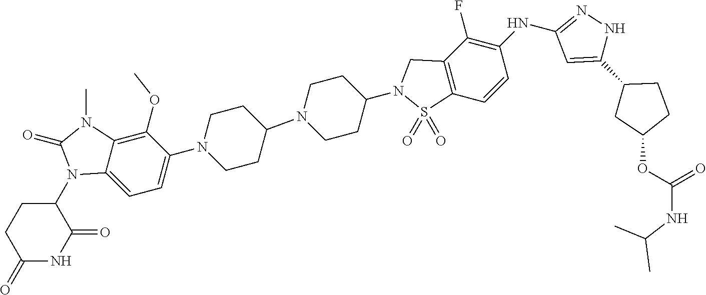 [SMILES: COc1c(N2CCC(N3CCC(N4Cc5c(ccc(Nc6cc([C@@H]7CC[C@H](OC(=O)NC(C)C)C7)[nH]n6)c5F)S4(=O)=O)CC3)CC2)ccc2c1n(C)c(=O)n2C1CCC(=O)NC1=O]

### Step 1: 4-bromo-3-methoxy-2-nitroaniline

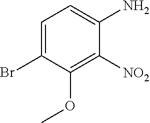

To a solution of 4-bromo-3-fluoro-2-nitroaniline (10 g, 42.7 mmol) in MeOH (500 mL) was added NaOMe (30%) (138 g, 769 mmol) at rt for overnight. The resulting mixture was concentrated under vacuum and extracted with DCM. The organic layers were washed with brine and concentrated under vacuum. The residue was purified by combi-flash (DCM/EA=100:0 to 87:13) to give the product (10 g, 95.2%) as yellow solid.

### Step 2: 5-bromo-4-methoxy-1,3-dihydro-2H-benzo[d]imidazol-2-one

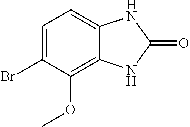

To a solution of 4-bromo-3-methoxy-2-nitroaniline (11 g, 44.7 mmol) in DMF (200 mL) were added B₂(OH)₄ (12 g, 134.14 mmol) and 4,4-Bipyridine (349 mg, 2.23 mmol) at 0° C. for 15 min under N₂. The CDI (14.48 g, 89.4 mmol) was added in it at rt for 1 h. The resulting solution was quenched with NH₄Cl solution and extracted with EA. The organic layers were washed brine and concentrated under vacuum. The filtrate was concentrated under vacuum. The residue was purified by combi-flash (DCM/MeOH=100:0 to 80:20) to give the product (5.2 g, 52.9%) as brown solid.

### Step 3: 1-(2,6-bis(benzyloxy)pyridin-3-yl)-5-bromo-4-methoxy-1,3-dihydro-2H-benzo[d]imidazol-2-one

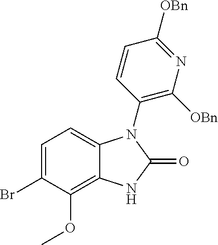 [SMILES: COc1c(Br)ccc2c1[nH]c(=O)n2-c1ccc(OCc2ccccc2)nc1OCc1ccccc1]

To a solution of 5-bromo-4-methoxy-1,3-dihydro-2H-benzo[d]imidazol-2-one (5.2 g, 21.48 mmol) in dioxane (100 mL) were added 2,6-bis(benzyloxy)-3-(4,4,5,5-tetramethyl-1,3,2-dioxaborolan-2-yl)pyridine (10.8 g, 33.23 mmol), Py (5.09 g, 64.44 mmol), Cu(OAc)₂ (4.27 g, 21.48 mmol) and 4AMS (2.5 g) at 80° C. for overnight under 02. The reaction was concentrated under vacuum. The residue was purified by combi-flash (PE/EA=100:0 to 67:37) to give the product (2 g, 10.5%) as light brown solid.

### Step 4: 1-(2,6-bis(benzyloxy)pyridin-3-yl)-5-bromo-4-methoxy-3-methyl-1,3-dihydro-2H-benzo[d]imidazol-2-one

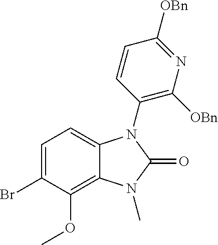 [SMILES: COc1c(Br)ccc2c1n(C)c(=O)n2-c1ccc(OCc2ccccc2)nc1OCc1ccccc1]

To a solution of 1-(2,6-bis(benzyloxy)pyridin-3-yl)-5-bromo-4-methoxy-1,3-dihydro-2H-benzo[d]imidazol-2-one (2.1 g, 3.95 mmol) in DMF (60 mL) were added K₂CO₃ (1.09 g, 7.91 mmol) and CH₃I (1.12 g, 7.91 mmol) at rt for 2 h. The resulting mixture was quenched with water and extracted with EA. The organic layers were washed with brine. The organic layers were concentrated under vacuum. The residue was purified by combi-flash (PE/EA=100:0 to 68:32) to give the product (0.95 g, 44.1%) as brown solid.

### Step 5: 1-(2,6-bis(benzyloxy)pyridin-3-yl)-4-methoxy-3-methyl-5-(1,4-dioxa-8-azaspiro[4.5]decan-8-yl)-1,3-dihydro-2H-benzo[d]imidazol-2-one

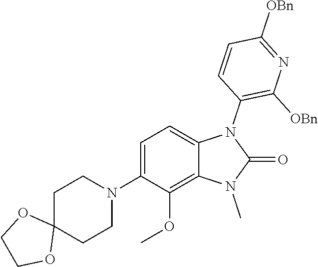 [SMILES: COc1c(N2CCC3(CC2)OCCO3)ccc2c1n(C)c(=O)n2-c1ccc(OCc2ccccc2)nc1OCc1ccccc1]

To a solution of 1-(2,6-bis(benzyloxy)pyridin-3-yl)-5-bromo-4-methoxy-3-methyl-1,3-dihydro-2H-benzo[d]imidazol-2-one (0.9 g, 1.65 mmol) in dry-dioxane (20 mL) were added 1,4-dioxa-8-azaspiro[4.5]decane (354 mg, 2.48 mmol), Pd-PEPPSI-IPentCl 2-methylpyridine (69.3 mg, 0.08 mmol) and Cs₂CO₃ (806 mg, 2.48 mmol) at 100° C. for overnight under N₂ atmosphere. The resulting mixture was concentrated under vacuum. The residue was purified by combi-flash (PE/EA=100:0 to 71:29) to give the product (0.7 g, 70.7%) as light brown semi-solid.

### Step 6: 3-(4-methoxy-3-methyl-2-oxo-5-(1,4-dioxa-8-azaspiro[4.5]decan-8-yl)-2,3-dihydro-1H-benzo[d]imidazol-1-yl)piperidine-2,6-dione

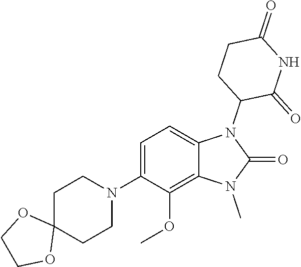 [SMILES: COc1c(N2CCC3(CC2)OCCO3)ccc2c1n(C)c(=O)n2C1CCC(=O)NC1=O]

To a solution of 1-(2,6-bis(benzyloxy)pyridin-3-yl)-4-methoxy-3-methyl-5-(1,4-dioxa-8-azaspiro[4.5]decan-8-yl)-1,3-dihydro-2H-benzo[d]imidazol-2-one (0.7 g, 1.15 mmol) in dry-THF (100 mL) was added Pd/C (0.7 g). The mixture was hydrogenated at room temperature for overnight under hydrogen atmosphere using a hydrogen balloon. The resulting mixture was filtered the filter cake was washed with THF (100 mL). The filtrate was concentrated under vacuum. The residue was without purified to give the product (0.46 g, 92.9%) as light brown solid.

### Step 7: 3-(4-methoxy-3-methyl-2-oxo-5-(4-oxopiperidin-1-yl)-2,3-dihydro-1H-benzo[d]imidazol-1-yl)piperidine-2,6-dione

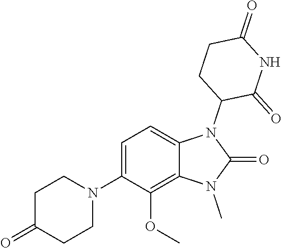 [SMILES: COc1c(N2CCC(=O)CC2)ccc2c1n(C)c(=O)n2C1CCC(=O)NC1=O]

To a solution of 3-(4-methoxy-3-methyl-2-oxo-5-(1,4-dioxa-8-azaspiro[4.5]decan-8-yl)-2,3-dihydro-1H-benzo[d]imidazol-1-yl)piperidine-2,6-dione (0.46 g, 1.07 mmol) in H₂SO₄ (10 mL) at 0° C. The reaction was stirred at rt for 2 h. The resulting mixture was quenched with ice-water (20 mL). The mixture was acidified to PH=7 with NaHCO₃ solution. The resulting mixture was extracted with EA. The organic layers were concentrated under vacuum. The resulting mixture was added ether (15 mL) stirred at rt for 1 h and filtered, the filter cake was washed with ether. The residue was without purified to give the product (344 mg, 83.5%) as yellow green solid. LCMS (ESI) m/e [M+1]⁺=387.

### Step 8: (1S,3R)-3-(3-((2-(1′-(1-(2,6-dioxopiperidin-3-yl)-4-methoxy-3-methyl-2-oxo-2,3-dihydro-1H-benzo[d]imidazol-5-yl)-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

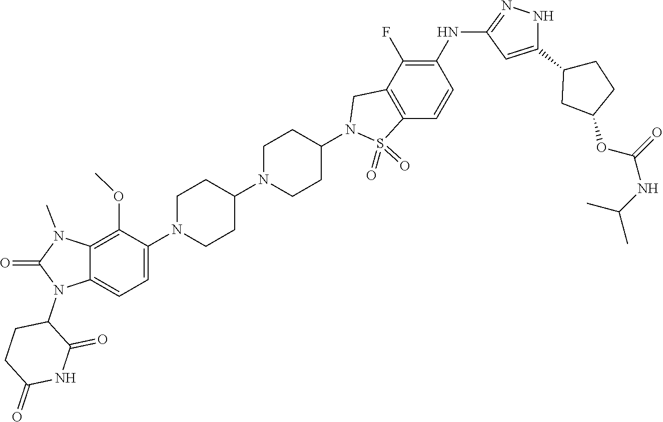 [SMILES: COc1c(N2CCC(N3CCC(N4Cc5c(ccc(Nc6cc([C@@H]7CC[C@H](OC(=O)NC(C)C)C7)[nH]n6)c5F)S4(=O)=O)CC3)CC2)ccc2c1n(C)c(=O)n2C1CCC(=O)NC1=O]

The titled compound was synthesized in the procedures similar to Example 39. ¹H NMR (500 MHz, DMSO-d₆) δ 11.99 (s, 1H), 11.06 (s, 1H), 8.75 (s, 1H), 8.27 (s, 1H), 7.51-7.49 (m, 1H), 6.97-6.85 (m, 1H), 6.78-6.73 (m, 1H), 6.70-6.45 (m, 1H), 5.82 (s, 1H), 5.31-5.17 (m, 1H), 5.10-4.87 (m, 1H), 4.53-4.49 (m, 2H), 3.91 (s, 3H), 3.62-3.50 (m, 1H), 3.48 (s, 3H), 3.46-3.23 (m, 3H), 3.00-2.95 (m, 3H), 2.94-2.83 (m, 1H), 2.75-2.41 (m, 4H), 2.48-2.45 (m, 1H), 2.39-2.26 (m, 2H), 2.07-1.92 (m, 4H), 1.91-1.75 (m, 6H), 1.74-1.49 (m, 5H), 1.02 (d, J=6.3 Hz, 6H). LC-MS (ESI): m/z [M+H]⁺=891.

### Example 186: (1S,3R)-3-(3-((2-(1-((1-(1-((S)-2,6-dioxopiperidin-3-yl)-8-fluoro-2-oxo-1,2,3,4-tetrahydro-5-oxa-1,2a-diazaacenaphthylen-6-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

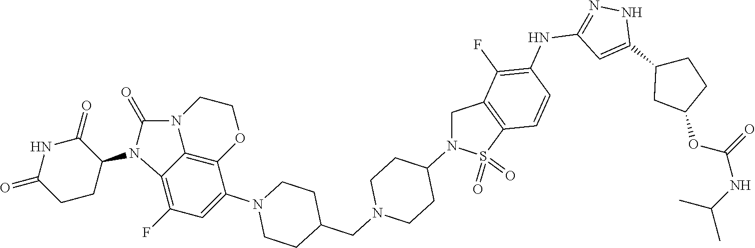 [SMILES: CC(C)NC(=O)O[C@H]1CC[C@@H](c2cc(Nc3ccc4c(c3F)CN(C3CCN(CC5CCN(c6cc(F)c7c8c6OCCn8c(=O)n7[C@H]6CCC(=O)NC6=O)CC5)CC3)S4(=O)=O)n[nH]2)C1]

### Step 1: 6-bromo-3,4-difluoro-2-nitrophenol

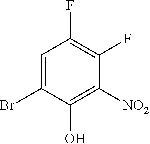 [SMILES: O=[N+]([O-])c1c(O)c(Br)cc(F)c1F]

To a solution of 2-bromo-4,5-difluorophenol (15 g, 71.8 mmol) in DCM (100 mL) were added HNO₃ (6.8 g, 107.7 mmol). The resulting solution was stirred for 1 h at −20° C. The resulting mixture was concentrated under vacuum. The residue was purified by combi-flash (EtOAc/PE=40%) to give the product (14 g, 76.80%) as red solid. LCMS (ESI) m/e [M+1]+=254

### Step 2: (2-(6-bromo-3,4-difluoro-2-nitrophenoxy)ethoxy)(tert-butyl)dimethylsilane

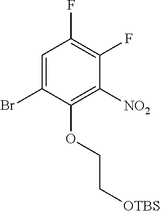 [SMILES: CC(C)(C)[Si](C)(C)OCCOc1c(Br)cc(F)c(F)c1[N+](=O)[O-]]

To a solution of 6-bromo-3,4-difluoro-2-nitrophenol (10 g, 39.3 mmol) in THF (100 mL) were added 2-((tert-butyldimethylsilyl)oxy)ethan-1-ol (10.3 g, 59.1 mmol), PPh₃ (15.5 g, 59.0 mmol), DIAD (2.6 g, 98.3 mmol). The resulting solution was stirred for 16 h at rt. The resulting mixture was concentrated under vacuum. The residue was purified by combi-flash (EtOAc/PE=50%) to give the product (13 g, 80.14%) as red solid. LCMS (ESI) m/e [M+1]+=412

### Step 3: 2,6-bis(benzyloxy)-N-(4-bromo-3-(2-((tert-butyldimethylsilyl)oxy)ethoxy)-6-fluoro-2-nitrophenyl)pyridin-3-amine

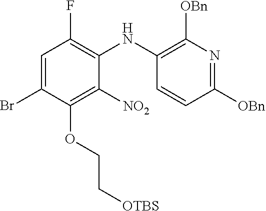 [SMILES: CC(C)(C)[Si](C)(C)OCCOc1c(Br)cc(F)c(Nc2ccc(OCc3ccccc3)nc2OCc2ccccc2)c1[N+](=O)[O-]]

To a solution of 2-bromo-4,5-difluorophenol (20 g, 36.4 mmol) in THF (300 mL) was added LiHMDS (1M in THF, 72.8 mL, 72.8 mmol) dropwise in 30 min at 0° C., the reaction solution was stirred for 1 h at 0° C., then to this was added (2-(6-bromo-3,4-difluoro-2-nitrophenoxy)ethoxy)(tert-butyl)dimethylsilane (15 g, 36.4 mmol) in THF (50 mL) dropwise in 20 min. The resulting solution was stirred for 3 h at rt. The reaction was quenched by the addition of sat·aq. NH₄Cl solution. The resulting solution was extracted with 3×200 mL of EtOAc. The resulting mixture was concentrated under vacuum. The residue was purified by combi-flash (EtOAc/PE=50%) to give the product (17 g, 66.8%) as red oil. LCMS (ESI) m/e [M+1]⁺=698

### Step 4: N1-(2,6-bis(benzyloxy)pyridin-3-yl)-4-bromo-3-(2-((tert-butyldimethylsilyl)oxy)ethoxy)-6-fluorobenzene-1,2-diamine

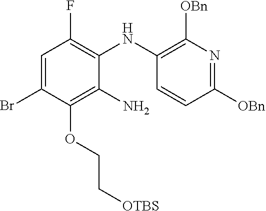 [SMILES: CC(C)(C)[Si](C)(C)OCCOc1c(Br)cc(F)c(Nc2ccc(OCc3ccccc3)nc2OCc2ccccc2)c1N]

To a solution of 2,6-bis(benzyloxy)-N-(4-bromo-3-(2-((tert-butyldimethylsilyl)oxy) ethoxy)-6-fluoro-2-nitrophenyl)pyridin-3-amine (17 g, 24.3 mmol) in MeOH:H2O=4:1 (600 mL) were added Fe (12.6 g, 121.6 mmol) and NH₄Cl (11.9 g, 121.6 mmol). The resulting solution was stirred for 3 h at 80° C. The resulting mixture was filtered, the filter cake was washed with DCM. The filtrate was extracted with 3×300 mL of DCM. The organic layers were concentrated under vacuum. The residue was purified by combi-flash (EtOAc/PE=46%) to give the product (14 g, 86.04%) as red oil. LCMS (ESI) m/e [M+1]⁺=668

### Step 5: 1-(2,6-bis(benzyloxy)pyridin-3-yl)-5-bromo-4-(2-((tert-butyldimethylsilyl)oxy)ethoxy)-7-fluoro-1,3-dihydro-2H-benzo[d]imidazol-2-one

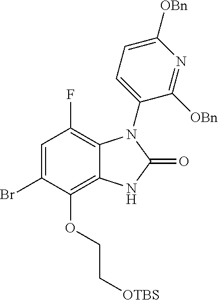 [SMILES: CC(C)(C)[Si](C)(C)OCCOc1c(Br)cc(F)c2c1[nH]c(=O)n2-c1ccc(OCc2ccccc2)nc1OCc1ccccc1]

To a solution of N¹-(2,6-bis(benzyloxy)pyridin-3-yl)-4-bromobenzene-1,2-diamine (13 g, 19.4 mmol) in ACN (100 mL) were added CDI (4.7 g, 29.1 mmol), DIEA (3.8 g, 29.1 mmol). The resulting solution was stirred for 16 h at 100° C. The resulting mixture was concentrated under vacuum. The residue was purified by combi-flash (EtOAc/PE=60%) to give the product (10.3 g, 76.3%) as pink solid. LCMS (ESI) m/e [M+1]⁺=694

### Step 6: 1-(2,6-bis(benzyloxy)pyridin-3-yl)-5-bromo-7-fluoro-4-(2-hydroxyethoxy)-1,3-dihydro-2H-benzo[d]imidazol-2-one

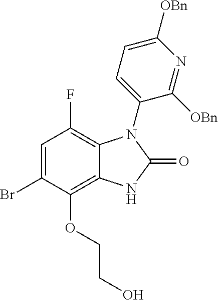 [SMILES: O=c1[nH]c2c(OCCO)c(Br)cc(F)c2n1-c1ccc(OCc2ccccc2)nc1OCc1ccccc1]

To a solution of 1-(2,6-bis(benzyloxy)pyridin-3-yl)-5-bromo-4-(2-((tert-butyldimethylsilyl)oxy)ethoxy)-7-fluoro-1,3-dihydro-2H-benzo[d]imidazol-2-one (10 g, 14.38 mmol) in DMF (100 mL) were added CsF (2.6 g, 28.77 mmol). The resulting solution was stirred for 16 h at 100° C. The resulting solution was extracted with 3×300 mL of EtOAc. The resulting mixture was concentrated under vacuum. The residue was purified by combi-flash (EtOAc/PE=80%) to give the product (7.2 g, 86.3%) as white solid. LCMS (ESI) m/e [M+1]⁺=580

### Step 7: 1-(2,6-bis(benzyloxy)pyridin-3-yl)-6-bromo-8-fluoro-3,4-dihydro-5-oxa-1,2a-diazaacenaphthylen-2(1H)-one

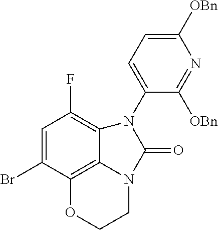 [SMILES: O=c1n2c3c(c(Br)cc(F)c3n1-c1ccc(OCc3ccccc3)nc1OCc1ccccc1)OCC2]

To a solution of 1-(2,6-bis(benzyloxy)pyridin-3-yl)-5-bromo-4-(2-((tert-butyldimethylsilyl)oxy)ethoxy)-7-fluoro-1,3-dihydro-2H-benzo[d]imidazol-2-one (7.1 g, 12.2 mmol) in THF (100 mL) were added DIAD (2.6 g, 18.4 mmol), PPh₃ (2.6 g, 18.4 mmol) at 0° C. The resulting solution was stirred for 16 h at rt. The resulting mixture was concentrated under vacuum. The residue was purified by combi-flash (EtOAc/PE=60%) to give the product (8 g, 99.0%) as white solid. LCMS (ESI) m/e [M+1]⁺=562

### Step 8: 1-(2,6-bis(benzyloxy)pyridin-3-yl)-6-(4-(dimethoxymethyl)piperidin-1-yl)-8-fluoro-3,4-dihydro-5-oxa-1,2a-diazaacenaphthylen-2(1H)-one

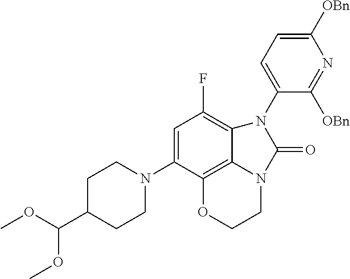 [SMILES: COC(OC)C1CCN(c2cc(F)c3c4c2OCCn4c(=O)n3-c2ccc(OCc3ccccc3)nc2OCc2ccccc2)CC1]

To a solution of 1-(2,6-bis(benzyloxy)pyridin-3-yl)-6-bromo-8-fluoro-3,4-dihydro-5-oxa-1,2a-diazaacenaphthylen-2(1H)-one (2 g, 3.6 mmol) in dioxane (80 mL) were added 4-(dimethoxymethyl)piperidine (858.6 g, 5.4 mmol), Pd-PEPPSI-IPentCl 2-methylpyridine (151.2 mg, 0.18 mmol) and Cs₂CO₃ (2.9 g, 9 mmol). The resulting solution was stirred for 24 h at 100° C. under nitrogen atmosphere. The resulting mixture was concentrated under vacuum. The residue was purified by combi-flash (EtOAc/PE=80%) to give the product (2 g, 87.7%) as yellow solid. LCMS (ESI) m/e [M+1]⁺=641

### Step 9: 3-(6-(4-(dimethoxymethyl)piperidin-1-yl)-8-fluoro-2-oxo-3,4-dihydro-5-oxa-1,2a-diazaacenaphthylen-1(2H)-yl)piperidine-2,6-dione

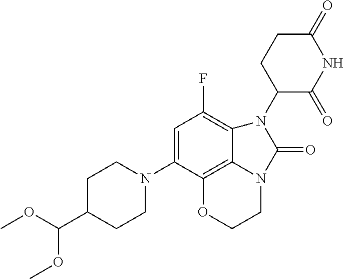 [SMILES: COC(OC)C1CCN(c2cc(F)c3c4c2OCCn4c(=O)n3C2CCC(=O)NC2=O)CC1]

To a solution of 1-(2,6-bis(benzyloxy)pyridin-3-yl)-6-(4-(dimethoxymethyl)piperidin-1-yl)-8-fluoro-3,4-dihydro-5-oxa-1,2a-diazaacenaphthylen-2(1H)-one (2 g, 3.1 mmol) in THF (30 mL) was added Pd/C (2 g, 10% wt). The resulting solution was stirred for 12 h at 50° C. under H₂ atmosphere. After filtration, the filtrate was concentrated under vacuum. The residue was the product (703 mg, 48.7%) as off-white solid. LCMS (ESI) m/e [M+1]⁺=463

### Step 10: 1-(1-(2,6-dioxopiperidin-3-yl)-8-fluoro-2-oxo-1,2,3,4-tetrahydro-5-oxa-1,2a-diazaacenaphthylen-6-yl)piperidine-4-carbaldehyde

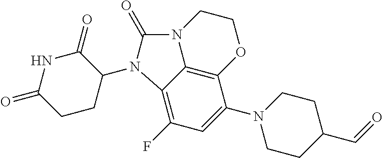 [SMILES: O=CC1CCN(c2cc(F)c3c4c2OCCn4c(=O)n3C2CCC(=O)NC2=O)CC1]

(S)-3-(6-(4-(dimethoxymethyl)piperidin-1-yl)-8-fluoro-2-oxo-3,4-dihydro-5-oxa-1,2a-diazaacenaphthylen-1(2H)-yl)piperidine-2,6-dione (500 mg, 1.079 mmol) was dissolved in formic acid (5 mL), stirred for 2 hours at room temperature. The resulting mixture was concentrated under reduce pressure, dried over in vacuum, to afford the product (400 mg, 88.8%). LC-MS (ESI): m/z [M+H]⁺=417.4.

### Step 11: (1S,3R)-3-(3-((2-(1-((1-(1-((S)-2,6-dioxopiperidin-3-yl)-8-fluoro-2-oxo-1,2,3,4-tetrahydro-5-oxa-1,2a-diazaacenaphthylen-6-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

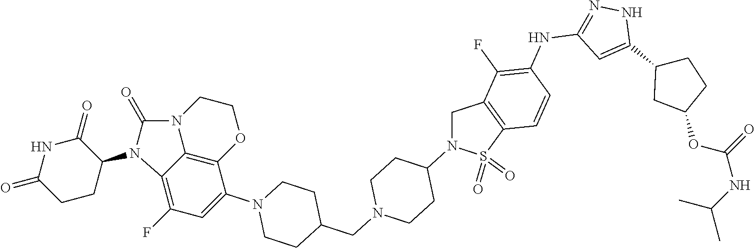 [SMILES: CC(C)NC(=O)O[C@H]1CC[C@@H](c2cc(Nc3ccc4c(c3F)CN(C3CCN(CC5CCN(c6cc(F)c7c8c6OCCn8c(=O)n7[C@H]6CCC(=O)NC6=O)CC5)CC3)S4(=O)=O)n[nH]2)C1]

The titled compound was synthesized in the procedures similar to Example 36. ¹H NMR (500 MHz, DMSO) δ 11.99 (s, 1H), 11.10 (s, 1H), 8.76 (s, 1H), 8.30 (t, J=8.0 Hz, 1H), 7.51 (d, J=8.6 Hz, 1H), 6.94 (d, J=7.3 Hz, 1H), 6.40 (d, J=13.6 Hz, 1H), 5.83 (s, 1H), 5.38 (s, 1H), 5.06-4.96 (m, 1H), 4.51 (s, 2H), 4.40-4.32 (m, 2H), 3.98-3.86 (m, 2H), 3.72-3.54 (m, 1H), 3.45-3.37 (m, 3H), 3.12-2.85 (m, 4H), 2.61-2.54 (m, 6H), 2.46-2.42 (m, 1H), 2.13-2.16 (m, 2H), 2.14-2.08 (m, 1H), 2.03 (s, 3H), 1.92-1.82 (m, 5H), 1.81-1.66 (m, 4H), 1.64-1.53 (m, 2H), 1.32-1.19 (m, 2H), 1.03 (d, J=6.3 Hz, 6H). LC-MS (ESI): m/z [M+H]⁺=921.4;

### Example 187: (1S,3R)-3-(3-((2-(1′-(1-((R)-2,6-dioxopiperidin-3-yl)-8-fluoro-2-oxo-1,2,3,4-tetrahydro-5-oxa-1,2a-diazaacenaphthylen-6-yl)-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

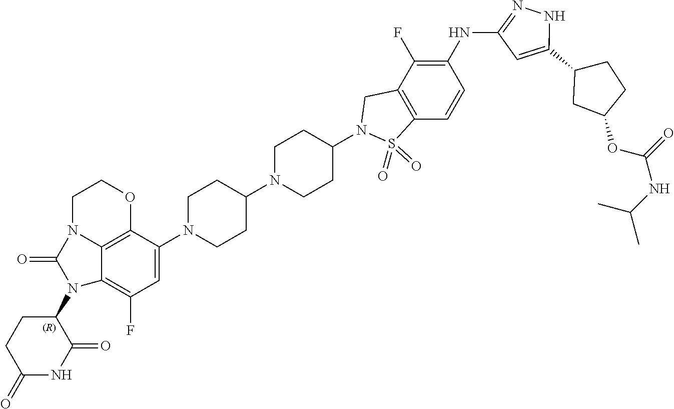

### Step 1: 1-(2,6-bis(benzyloxy)pyridin-3-yl)-8-fluoro-6-(1,4-dioxa-8-azaspiro[4.5]decan-8-yl)-3,4-dihydro-5-oxa-1,2a-diazaacenaphthylen-2(1H)-one

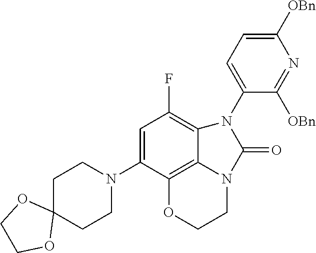 [SMILES: O=c1n2c3c(c(N4CCC5(CC4)OCCO5)cc(F)c3n1-c1ccc(OCc3ccccc3)nc1OCc1ccccc1)OCC2]

To a solution of 1-(2,6-bis(benzyloxy)pyridin-3-yl)-6-bromo-8-fluoro-3,4-dihydro-5-oxa-1,2a-diazaacenaphthylen-2(1H)-one (3 g, 5.3 mmol) in dioxane (80 mL) were added 1,4-dioxa-8-azaspiro[4.5]decane (1.4 g, 8.0 mmol), Pd-PEPPSI-IPentCl 2-methylpyridine (223 mg, 0.265 mmol) and Cs₂CO₃ (4.3 g, 13.3 mmol). The resulting solution was stirred for 24 h at 100° C. under nitrogen atmosphere. The resulting mixture was concentrated under vacuum. The residue was purified by combi-flash (EtOAc/PE=80%) to give the product (2.6 g, 78.0%) as yellow solid. LCMS (ESI) m/e [M+1]⁺=625

### Step 2: 3-(8-fluoro-2-oxo-6-(1,4-dioxa-8-azaspiro[4.5]decan-8-yl)-3,4-dihydroacenaphthylen-1(2H)-yl)piperidine-2,6-dione

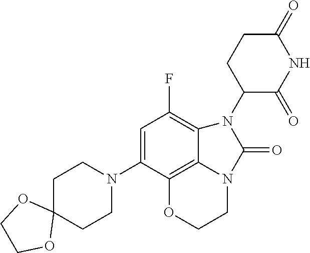 [SMILES: O=C1CCC(n2c(=O)n3c4c(c(N5CCC6(CC5)OCCO6)cc(F)c42)OCC3)C(=O)N1]

To a solution of 1-(2,6-bis(benzyloxy)pyridin-3-yl)-8-fluoro-6-(1,4-dioxa-8-azaspiro[4.5]decan-8-yl)-3,4-dihydro-5-oxa-1,2a-diazaacenaphthylen-2(1H)-one (2.5 g, 4.16 mmol) in THF (30 mL) was added Pd/C (2.6 g, 10% wt). The resulting solution was stirred for 12 h at 50° C. under H₂ atmosphere. The resulting mixture was filtered, the filtrate was concentrated under vacuum. The residue was the product (2 g, 90%) as off-white solid. LCMS (ESI) m/e [M+1]⁺=447

### Step 3: 3-(8-fluoro-2-oxo-6-(4-oxopiperidin-1-yl)-3,4-dihydro-5-oxa-1,2a-diazaacenaphthylen-1(2H)-yl)piperidine-2,6-dione

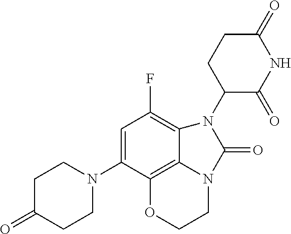 [SMILES: O=C1CCN(c2cc(F)c3c4c2OCCn4c(=O)n3C2CCC(=O)NC2=O)CC1]

The solution of 3-(8-fluoro-2-oxo-6-(1,4-dioxa-8-azaspiro[4.5]decan-8-yl)-3,4-dihydroacenaphthylen-1(2H)-yl)piperidine-2,6-dione (2 g, 4.8 mmol) in H₂SO₄ (20 mL) at 0° C. The resulting solution was stirred for 3 h at r.t. The mixture was basified to pH=5 with Na₂CO₃ and extracted by EA. The combined organic phase was concentrated under reduced pressure. The residue was the product (903.7 mg, 50.1%) as pink solid. LCMS (ESI) m/e [M+1]⁺=403

### Step 4: (1S,3R)-3-(3-((2-(1′-(1-((R)-2,6-dioxopiperidin-3-yl)-8-fluoro-2-oxo-1,2,3,4-tetrahydro-5-oxa-1,2a-diazaacenaphthylen-6-yl)-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

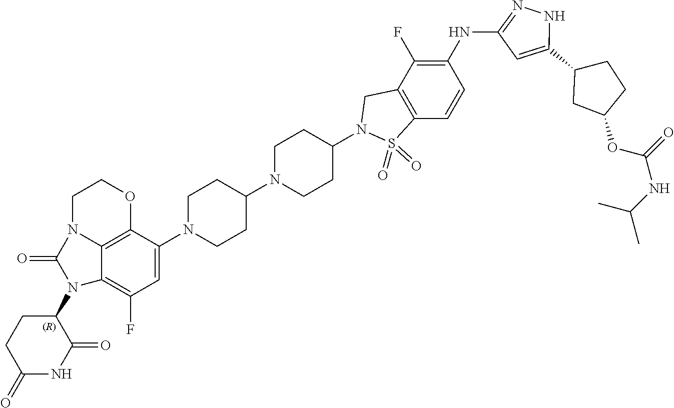

The racemic product was synthesized in the procedures similar to Example 178, which was further separated by Chiral Prep-HPLC to afford the Example 187(100% ee); Retention time: 8.198 min. ¹H NMR (500 MHz, DMSO-d₆) δ 11.99 (s, 1H), 11.11 (s, 1H), 8.76 (s, 1H), 8.29 (s, 1H), 7.51 (d, J=10 Hz, 1H), 6.94 (d, J=10 Hz, 1H), 5.83 (s, 1H), 5.45-5.38 (m, 1H), 5.07-4.95 (m, 1H), 4.51 (s, 1H), 4.40-4.34 (m, 1H), 3.98-3.89 (m, 1H), 3.62-3.54 (m, 2H), 3.50-3.38 (m, 4H), 3.11-3.02 (m, 1H), 3.01-2.93 (m, 3H), 2.62-2.54 (m, 4H), 2.48-2.45 (m, 1H), 2.43-2.27 (m, 1H), 2.14-1.98 (m, 2H), 1.97-1.88 (m, 3H), 1.88-1.76 (m, 4H), 1.75-1.66 (m, 2H), 1.65-1.54 (m, 5H), 1.07-0.99 (m, 6H). LC-MS (ESI): m/z [M+H]⁺=907.

Chiral analytical method: Column: I-Cellulose-5, 4.6 mm×250 mm, 5 um; Mobile phase: A for MeBE (0.1% 2M NH3 MeOH) and B for DCM/MeOH (50:50); Gradient: Mobile Phase A: Mobile Phase B=30:70 (v/v); HPLC Equipment: HPLC-Agilent, Back pressure: 100 bar; Column temperature: 35° C.

Chiral Prep-HPLC Condition: Column: I-Cellulose-5, 20 mm×250 mm, 5 um; Mobile phase: A for MeBE (0.1% 2M NH3 MeOH) and B for DCM/MeOH (50:50); Gradient: Mobile Phase A: Mobile Phase B=30:70 (v/v); Flow Rate: 20 mL/min, Wave Length: UV 200 nm and 270 nm, Prep-HPLC Equipment: Prep-HPLC-Gilson, Back pressure: 100 bar; Column temperature: 25° C.

### Example 188: (1S,3R)-3-(3-((2-(1-((1-(1-(2,6-dioxopiperidin-3-yl)-4-fluoro-3-methyl-2-oxo-2,3-dihydro-1H-benzo[d]imidazol-5-yl)piperidin-4-yl)methyl)azetidin-3-yl)-4-fluoro-1,1-dioxido-2,3-dihydrobenzo[d]isothiazol-5-yl)amino)-1H-pyrazol-5-yl)cyclopentyl isopropylcarbamate

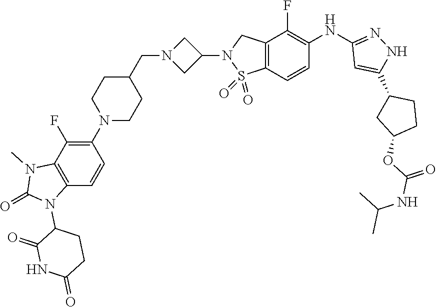 [SMILES: CC(C)NC(=O)O[C@H]1CC[C@@H](c2cc(Nc3ccc4c(c3F)CN(C3CN(CC5CCN(c6ccc7c(c6F)n(C)c(=O)n7C6CCC(=O)NC6=O)CC5)C3)S4(=O)=O)n[nH]2)C1]

The racemic product was synthesized in the procedures similar to Example 46. ¹H NMR (500 MHz, DMSO) δ 12.04 (s, 1H), 11.10 (s, 1H), 9.84 (d, J=98.7 Hz, 1H), 8.91 (s, 1H), 8.33 (s, 1H), 7.61 (d, J=8.8 Hz, 1H), 6.93 (d, J=7.1 Hz, 1H), 6.86 (d, J=8.5 Hz, 1H), 6.77-6.73 (m, 1H), 5.85 (s, 1H), 5.34 (dd, J=12.7, 5.1 Hz, 1H), 5.00 (s, 1H), 4.81-4.78 (m, 2H), 4.68 (s, 1H), 4.61-4.47 (m, 3H), 4.41 (s, 1H), 3.47 (s, 3H), 3.30-3.24 (m, 4H), 3.07 (s, 1H), 2.92-2.86 (m, 1H), 2.68-2.60 (m, 5H), 2.36 (s, 1H), 2.08-1.96 (m, 2H), 1.92 (s, 1H), 1.77-1.72 (m, 4H), 1.59 (s, 1H), 1.43 (s, 2H), 1.04-1.03 (m, 6H). LC-MS (ESI): m/z [M+H]+=865.6.

### Example 189: (1S,3R)-3-(3-((2-(1-(1-(1-(2,6-dioxopiperidin-3-yl)-4-fluoro-3-methyl-2-oxo-2,3-dihydro-1H-benzo[d]imidazol-5-yl)piperidin-4-yl)azetidin-3-yl)-4-fluoro-1,1-dioxido-2,3-dihydrobenzo[d]isothiazol-5-yl)amino)-1H-pyrazol-5-yl)cyclopentyl isopropylcarbamate

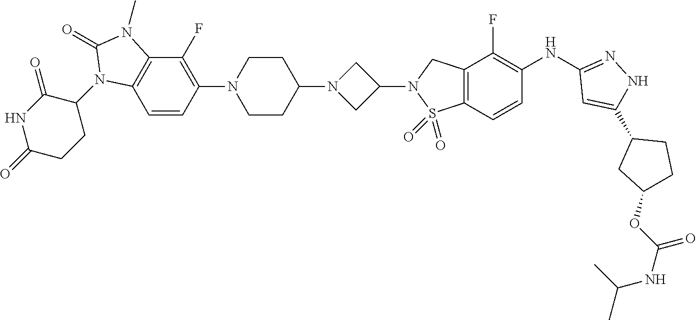 [SMILES: CC(C)NC(=O)O[C@H]1CC[C@@H](c2cc(Nc3ccc4c(c3F)CN(C3CN(C5CCN(c6ccc7c(c6F)n(C)c(=O)n7C6CCC(=O)NC6=O)CC5)C3)S4(=O)=O)n[nH]2)C1]

The racemic product was synthesized in the procedures similar to Example 45. ¹H NMR (500 MHz, DMSO) δ 12.01 (s, 1H), 11.10 (s, 1H), 8.81 (s, 1H), 8.30 (s, 1H), 7.54 (d, J=8.6 Hz, 1H), 6.93 (s, 1H), 6.85 (d, J=8.4 Hz, 1H), 6.75 (t, J=8.1 Hz, 1H), 5.84 (s, 1H), 5.33 (dd, J=12.9, 5.4 Hz, 1H), 4.99 (s, 1H), 4.61 (s, 2H), 4.17-4.09 (m, 1H), 3.60-3.51 (m, 3H), 3.47 (s, 3H), 3.20-3.18 (m, 2H), 3.07 (s, 1H), 2.91-2.85 (m, 1H), 2.71-2.60 (m, 7H), 2.26 (s, 1H), 2.05-1.98 (m, 2H), 1.90 (s, 1H), 1.79-1.68 (m, 4H), 1.60 (s, 1H), 1.40-1.34 (m, 2H), 1.04-1.02 (m, 6H). LC-MS (ESI): m/z [M+H]⁺=851.73.

### Example 190: (1S,3R)-3-(3-((2-(4-(4-(1-((S)-2,6-dioxopiperidin-3-yl)-4-fluoro-3-methyl-2-oxo-2,3-dihydro-1H-benzo[d]imidazol-5-yl)piperidin-1-yl)cyclohexyl)-4-fluoro-1,1-dioxido-2,3-dihydrobenzo[d]isothiazol-5-yl)amino)-1H-pyrazol-5-yl)cyclopentyl isopropylcarbamate

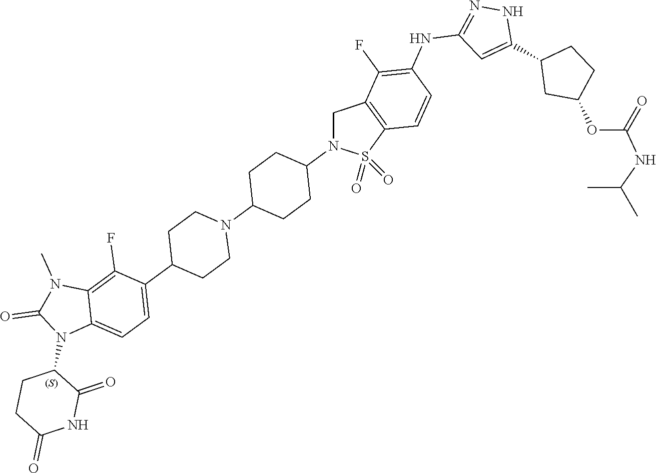

The racemic compound was synthesized in the procedures similar to Example 45, which was further separated by Chiral Prep-HPLC to afford Example 45 (100% ee); Retention time: 11.97 min. ¹H NMR (500 MHz, DMSO-d₆) δ 11.99 (s, 1H), 11.11 (s, 1H), 8.79-8.75 (m, 1H), 8.30 (s, 1H), 7.51 (d, J=10 Hz, 1H), 6.92-6.85 (m, 3H), 5.83 (s, 1H), 5.42-5.35 (m, 1H), 5.02-4.97 (m, 1H), 4.58-4.49 (m, 2H), 3.65-3.58 (m, 1H), 3.48 (s, 3H), 3.18-3.07 (m, 2H), 2.91-2.78 (m, 3H), 2.57-2.50 (m, 3H), 2.36-2.25 (m, 2H), 2.18-2.07 (m, 2H), 2.05-1.99 (m, 3H), 1.98-1.85 (m, 3H), 1.78-1.62 (m, 8H), 1.50-1.45 (m, 1H), 1.15-1.01 (m, 6H). LC-MS (ESI): m/z [M+H]⁺=878.

Chiral analytical method: i-Cellulose-5, 4.6 mm×250 mm, 5 um; Mobile phase: A for MtBE (0.1% 2M NH3 MeOH) and B for DCM:MeOH (50:50); Gradient: Mobile Phase A: Mobile Phase B=30:70 (v/v); HPLC Equipment: HPLC-Agilent, Back pressure: 100 bar; Column temperature: 35° C.

Chiral Prep-HPLC Condition: Column: i-Cellulose-5, 250 mm×20 mm, 5 um; Mobile phase: A for MtBE (0.1% 2M NH3 MeOH) and B for DCM:MeOH (50:50); Gradient: Mobile Phase A: Mobile Phase B=30:70 (v/v); Flow Rate: 20 mL/min, Wave Length: UV 200 nm and 270 nm, Prep-HPLC Equipment: Prep-HPLC-Gilson, Back pressure: 100 bar; Column temperature: 25° C.

### Example 191a and 191b: (1S,3R)-3-(3-((2-((1R,5S,6r)-3-((1-(1-(2,6-dioxopiperidin-3-yl)-4-fluoro-3-methyl-2-oxo-2,3-dihydro-1H-benzo[d]imidazol-5-yl)piperidin-4-yl)methyl)-3-azabicyclo[3.1.1]heptan-6-yl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-((1R,5S,6s)-3-((1-(1-(2,6-dioxopiperidin-3-yl)-4-fluoro-3-methyl-2-oxo-2,3-dihydro-1H-benzo[d]imidazol-5-yl)piperidin-4-yl)methyl)-3-azabicyclo[3.1.1]heptan-6-yl)-4-fluoro-1,1-dioxido-2,3-dihydrobenzo[d]isothiazol-5-yl)amino)-1H-pyrazol-5-yl)cyclopentyl isopropylcarbamate

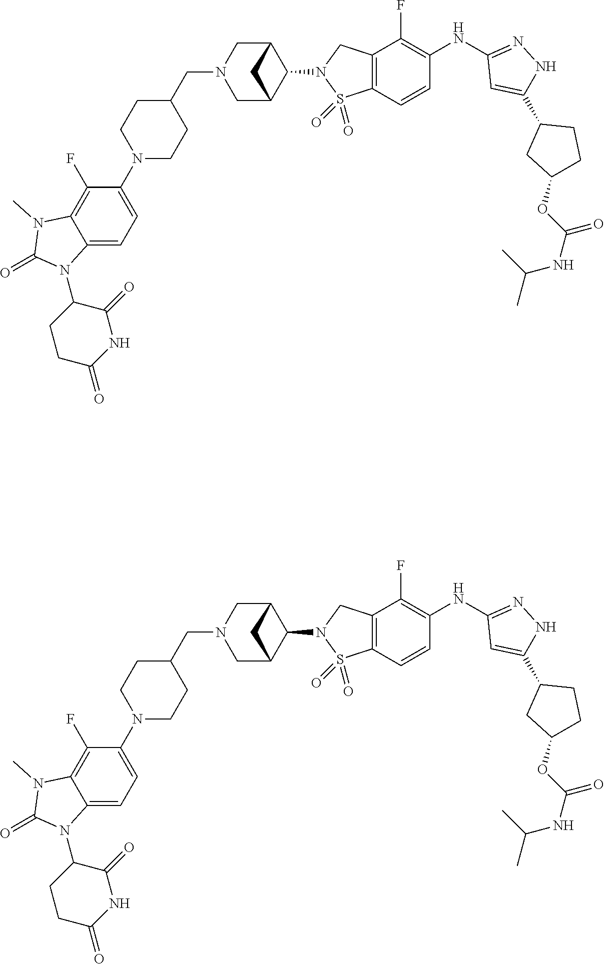 [SMILES: CC(C)NC(=O)O[C@H]1CC[C@@H](c2cc(Nc3ccc4c(c3F)CN([C@H]3[C@@H]5C[C@H]3CN(CC3CCN(c6ccc7c(c6F)n(C)c(=O)n7C6CCC(=O)NC6=O)CC3)C5)S4(=O)=O)n[nH]2)C1 ; CC(C)NC(=O)O[C@H]1CC[C@@H](c2cc(Nc3ccc4c(c3F)CN([C@@H]3[C@@H]5C[C@H]3CN(CC3CCN(c6ccc7c(c6F)n(C)c(=O)n7C6CCC(=O)NC6=O)CC3)C5)S4(=O)=O)n[nH]2)C1]

### Step 1: tert-butyl 6-(5-bromo-4-fluoro-1,1-dioxidobenzo[d]isothiazol-2(3H)-yl)-3-azabicyclo[3.1.1]heptane-3-carboxylate

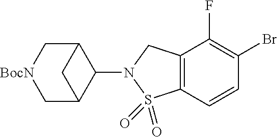 [SMILES: CC(C)(C)OC(=O)N1CC2CC(C1)C2N1Cc2c(ccc(Br)c2F)S1(=O)=O]

The titled compound was synthesized in the procedures similar to Example 1. LC-MS (ESI): m/z [M+H]⁺=461.2

### Step 2: tert-butyl (1R,5S,6s)-6-(4-fluoro-5-((5-((1R,3S)-3-((isopropylcarbamoyl)oxy)cyclopentyl)-1H-pyrazol-3-yl)amino)-1,1-dioxidobenzo[d]isothiazol-2(3H)-yl)-3-azabicyclo[3.1.1]heptane-3-carboxylate

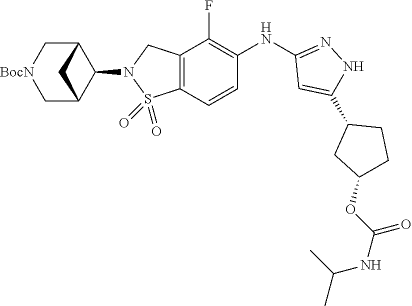 [SMILES: CC(C)NC(=O)O[C@H]1CC[C@@H](c2cc(Nc3ccc4c(c3F)CN([C@@H]3[C@@H]5C[C@H]3CN(C(=O)OC(C)(C)C)C5)S4(=O)=O)n[nH]2)C1]

The titled compound was synthesized in the procedures similar to Example 1. LC-MS (ESI): m/z [M+H]⁺=633.4

### Step 3: (1S,3R)-3-(3-((2-((1R,5S,6s)-3-azabicyclo[3.1.1]heptan-6-yl)-4-fluoro-1,1-dioxido-2,3-dihydrobenzo[d]isothiazol-5-yl)amino)-1H-pyrazol-5-yl)cyclopentyl isopropylcarbamate

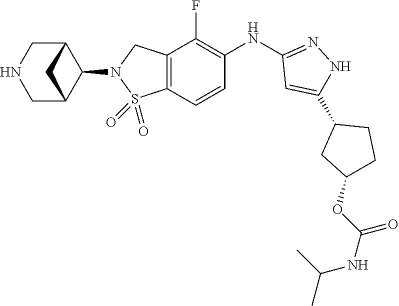 [SMILES: CC(C)NC(=O)O[C@H]1CC[C@@H](c2cc(Nc3ccc4c(c3F)CN([C@H]3[C@@H]5CNC[C@H]3C5)S4(=O)=O)n[nH]2)C1]

The titled compound was synthesized in the procedures similar to Example 1. LC-MS (ESI): m/z [M+H]⁺=533.6

### Step 4: (1S,3R)-3-(3-((2-((1R,5S,6r)-3-((1-(1-(2,6-dioxopiperidin-3-yl)-4-fluoro-3-methyl-2-oxo-2,3-dihydro-1H-benzo[d]imidazol-5-yl)piperidin-4-yl)methyl)-3-azabicyclo[3.1.1]heptan-6-yl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-((1R,5S,6s)-3-((1-(1-(2,6-dioxopiperidin-3-yl)-4-fluoro-3-methyl-2-oxo-2,3-dihydro-1H-benzo[d]imidazol-5-yl)piperidin-4-yl)methyl)-3-azabicyclo[3.1.1]heptan-6-yl)-4-fluoro-1,1-dioxido-2,3-dihydrobenzo[d]isothiazol-5-yl)amino)-1H-pyrazol-5-yl)cyclopentyl isopropylcarbamate

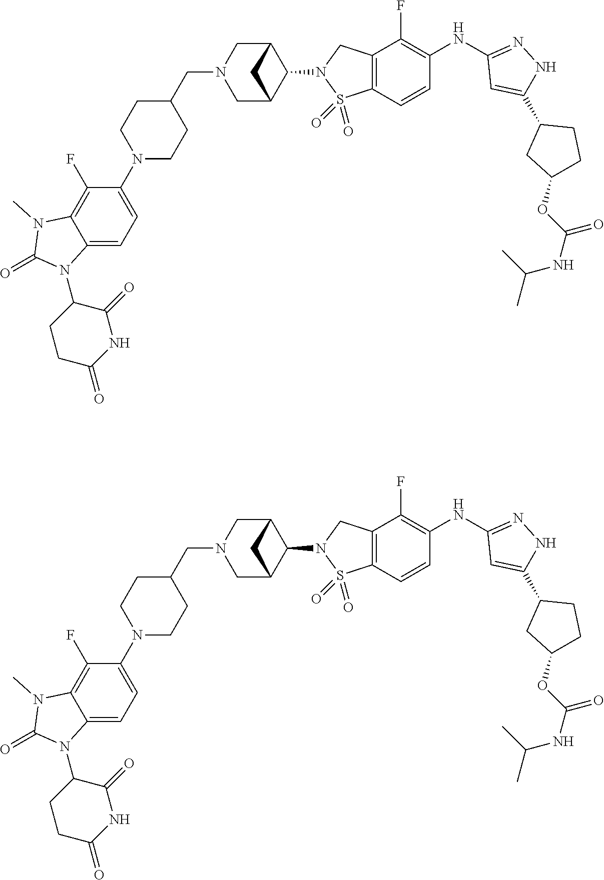 [SMILES: CC(C)NC(=O)O[C@H]1CC[C@@H](c2cc(Nc3ccc4c(c3F)CN([C@H]3[C@@H]5C[C@H]3CN(CC3CCN(c6ccc7c(c6F)n(C)c(=O)n7C6CCC(=O)NC6=O)CC3)C5)S4(=O)=O)n[nH]2)C1 ; CC(C)NC(=O)O[C@H]1CC[C@@H](c2cc(Nc3ccc4c(c3F)CN([C@@H]3[C@@H]5C[C@H]3CN(CC3CCN(c6ccc7c(c6F)n(C)c(=O)n7C6CCC(=O)NC6=O)CC3)C5)S4(=O)=O)n[nH]2)C1]

The titled compound was synthesized in the procedures similar to Example 36 in a racemic form, which was further separated by Prep-HPLC to give:

Enantiomer 1 (Example 191a): ¹H NMR (500 MHz, DMSO-d₆) δ 11.99 (s, 1H), 11.06 (s, 1H), 8.77 (s, 1H), 8.29 (s, 1H), 7.51 (d, J=10 Hz, 1H), 6.95-6.89 (m, 1H), 6.83-6.80 (m, 1H), 6.79-6.73 (m, 1H), 6.66-6.60 (m, 1H), 5.83 (s, 1H), 5.32-5.25 (m, 1H), 5.01-4.94 (m, 1H), 4.52 (s, 2H), 3.62-3.53 (m, 2H), 3.28-3.39 (m, 1H), 3.30 (s, 3H), 3.10-3.01 (m, 1H), 2.93-2.84 (m, 3H), 2.71-2.51 (m, 4H), 2.46-2.43 (m, 1H), 2.21-2.15 (m, 2H), 2.08-1.95 (m, 5H), 1.95-1.84 (m, 4H), 1.83-1.77 (m, 2H), 1.74-1.67 (m, 2H), 1.66-1.54 (m, 2H), 1.30-1.17 (m, 6H). LC-MS (ESI): m/z [M+H]⁺=905.6

Enantiomer 2 (Example 191b: ¹H NMR (500 MHz, DMSO-d₆) δ 11.99 (s, 1H), 11.06 (s, 1H), 8.77 (s, 1H), 8.29 (s, 1H), 7.51 (d, J=10 Hz, 1H), 6.95-6.89 (m, 1H), 6.83-6.80 (m, 1H), 6.79-6.73 (m, 1H), 6.66-6.60 (m, 1H), 5.83 (s, 1H), 5.32-5.25 (m, 1H), 5.01-4.94 (m, 1H), 4.52 (s, 2H), 3.62-3.53 (m, 2H), 3.28-3.39 (m, 1H), 3.30 (s, 3H), 3.10-3.01 (m, 1H), 2.93-2.84 (m, 3H), 2.71-2.51 (m, 4H), 2.46-2.43 (m, 1H), 2.21-2.15 (m, 2H), 2.08-1.95 (m, 5H), 1.95-1.84 (m, 4H), 1.83-1.77 (m, 2H), 1.74-1.67 (m, 2H), 1.66-1.54 (m, 2H), 1.30-1.17 (m, 6H). LC-MS (ESI): m/z [M+H]⁺=905.6

### Example 192a and 192b and 192c: (1S,3R)-3-(3-((2-((1R,4S)-4-((S)-3-(4-(1-((S)-2,6-dioxopiperidin-3-yl)-4-fluoro-3-methyl-2-oxo-2,3-dihydro-1H-benzo[d]imidazol-5-yl)piperazin-1-yl)pyrrolidin-1-yl)cyclohexyl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-((1R,4S)-4-((S)-3-(4-(1-((R)-2,6-dioxopiperidin-3-yl)-4-fluoro-3-methyl-2-oxo-2,3-dihydro-1H-benzo[d]imidazol-5-yl)piperazin-1-yl)pyrrolidin-1-yl)cyclohexyl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-((1S,4R)-4-((S)-3-(4-(1-((R)-2,6-dioxopiperidin-3-yl)-4-fluoro-3-methyl-2-oxo-2,3-dihydro-1H-benzo[d]imidazol-5-yl)piperazin-1-yl)pyrrolidin-1-yl)cyclohexyl)-4-fluoro-1,1-dioxido-2,3-dihydrobenzo[d]isothiazol-5-yl)amino)-1H-pyrazol-5-yl)cyclopentyl isopropylcarbamate

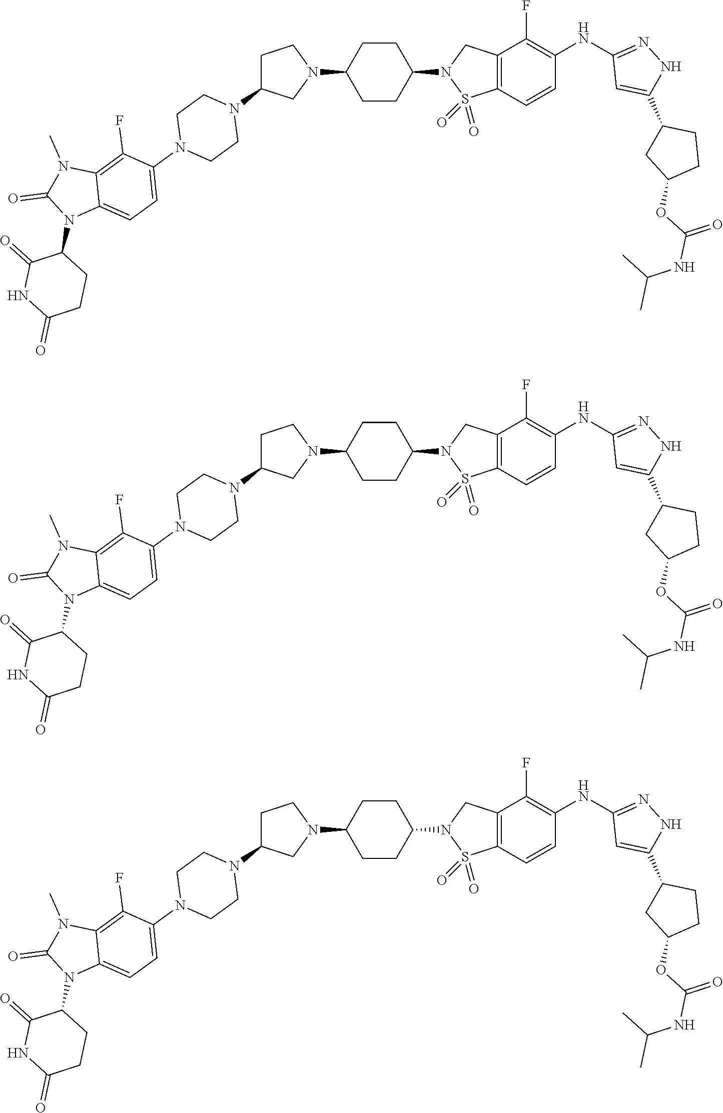 [SMILES: CC(C)NC(=O)O[C@H]1CC[C@@H](c2cc(Nc3ccc4c(c3F)CN([C@H]3CC[C@@H](N5CC[C@H](N6CCN(c7ccc8c(c7F)n(C)c(=O)n8[C@H]7CCC(=O)NC7=O)CC6)C5)CC3)S4(=O)=O)n[nH]2)C1 ; CC(C)NC(=O)O[C@H]1CC[C@@H](c2cc(Nc3ccc4c(c3F)CN([C@H]3CC[C@@H](N5CC[C@H](N6CCN(c7ccc8c(c7F)n(C)c(=O)n8[C@@H]7CCC(=O)NC7=O)CC6)C5)CC3)S4(=O)=O)n[nH]2)C1 ; CC(C)NC(=O)O[C@H]1CC[C@@H](c2cc(Nc3ccc4c(c3F)CN([C@H]3CC[C@H](N5CC[C@H](N6CCN(c7ccc8c(c7F)n(C)c(=O)n8[C@@H]7CCC(=O)NC7=O)CC6)C5)CC3)S4(=O)=O)n[nH]2)C1]

### Step 1: tert-butyl (3S)-3-(4-(1-(2,6-bis(benzyloxy)pyridin-3-yl)-4-fluoro-3-methyl-2-oxo-2,3-dihydro-1H-benzo[d]imidazol-5-yl)piperazin-1-yl)pyrrolidine-1-carboxylate

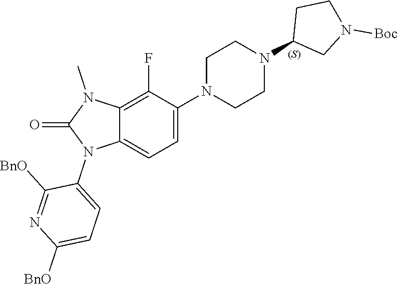

To a mixture of t 1-(2,6-bis(benzyloxy)pyridin-3-yl)-5-bromo-4-fluoro-3-methyl-1,3-dihydro-2H-benzo[d]imidazol-2-one (1 g, 1.87 mmol) and tert-butyl (S)-3-(piperazin-1-yl)pyrrolidine-1-carboxylate (478 mg, 1.87 mmol) in andydrous 1,4-dioxane (20 mL) was added Pd-PEPPSI (182 mg, 0.19 mmol) and Cs₃CO₃ (1.22 g, 3.74 mmol). The reaction mixture was stirred under nitrogen atmosphere at 90° C. for 16 h. The mixture was filtered, and the filtrate was concentrated under reduced pressure. The residue was purified by silica gel column chromatography, eluting with DCM/MeOH (1:20) to afford the product (720 mg, 54% yield). LC-MS (ESI): m/z [M+H]⁺=709.7.

### Step 2: tert-butyl (3S)-3-(4-(1-(2,6-dioxopiperidin-3-yl)-4-fluoro-3-methyl-2-oxo-2,3-dihydro-1H-benzo[d]imidazol-5-yl)piperazin-1-yl)pyrrolidine-1-carboxylate

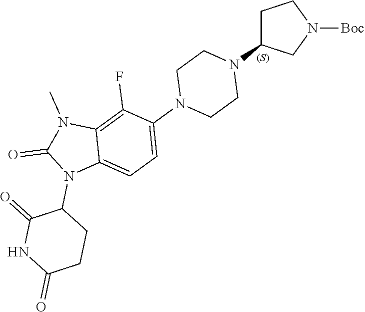

To a solution of tert-butyl (3S)-3-(4-(1-(2,6-bis(benzyloxy)pyridin-3-yl)-4-fluoro-3-methyl-2-oxo-2,3-dihydro-1H-benzo[d]imidazol-5-yl)piperazin-1-yl)pyrrolidine-1-carboxylate (720 mg, 0.83 mmol) in THF (20 mL) was added Pd/C (10%, wet, 500 mg). The resulting mixture was stirred at room temperature under a hydrogen balloon for 16 h. The mixture was filtered, and the filter cake was washed with MeOH (50 mL×3). The filtrate was concentrated under reduced pressure to afford the product (310 mg, 57% yield). LC-MS (ESI): m/z [M+H]+=531.5.

### Step 3: 3-(4-fluoro-3-methyl-2-oxo-5-(4-((S)-pyrrolidin-3-yl)piperazin-1-yl)-2,3-dihydro-1H-benzo[d]imidazol-1-yl)piperidine-2,6-dione

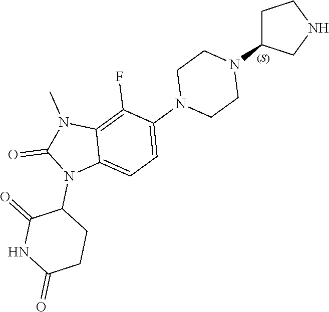

A solution of tert-butyl (3S)-3-(4-(1-(2,6-dioxopiperidin-3-yl)-4-fluoro-3-methyl-2-oxo-2,3-dihydro-1H-benzo[d]imidazol-5-yl)piperazin-1-yl)pyrrolidine-1-carboxylate (310 mg, 0.58 mmol) in DCM (2 mL) was treated with triflic acid (2 mL) dropwise. The resulting mixture was stirred at room temperature for 2 h. The reaction mixture was quenched and basified to pH=8 with sat. aq NaHCO₃ at 0° C. The layers separated and the aqueous layer was extracted with DCM/MeOH=20/1 (50 mL×3). The combined organic layers were washed with brine (50 mL) and dried over anhydrous Na₂SO₄. After filtration, the filtrate was concentrated under reduced pressure to afford the product (210 mg, 83% yield). LC-MS (ESI): m/z [M+H]⁺=431.4.

### Step 4: (1S,3R)-3-(3-((2-((1R,4S)-4-((S)-3-(4-(1-((S)-2,6-dioxopiperidin-3-yl)-4-fluoro-3-methyl-2-oxo-2,3-dihydro-1H-benzo[d]imidazol-5-yl)piperazin-1-yl)pyrrolidin-1-yl)cyclohexyl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-((1R,4S)-4-((S)-3-(4-(1-((R)-2,6-dioxopiperidin-3-yl)-4-fluoro-3-methyl-2-oxo-2,3-dihydro-1H-benzo[d]imidazol-5-yl)piperazin-1-yl)pyrrolidin-1-yl)cyclohexyl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-((1S,4R)-4-((S)-3-(4-(1-((R)-2,6-dioxopiperidin-3-yl)-4-fluoro-3-methyl-2-oxo-2,3-dihydro-1H-benzo[d]imidazol-5-yl)piperazin-1-yl)pyrrolidin-1-yl)cyclohexyl)-4-fluoro-1,1-dioxido-2,3-dihydrobenzo[d]isothiazol-5-yl)amino)-1H-pyrazol-5-yl)cyclopentyl isopropylcarbamate

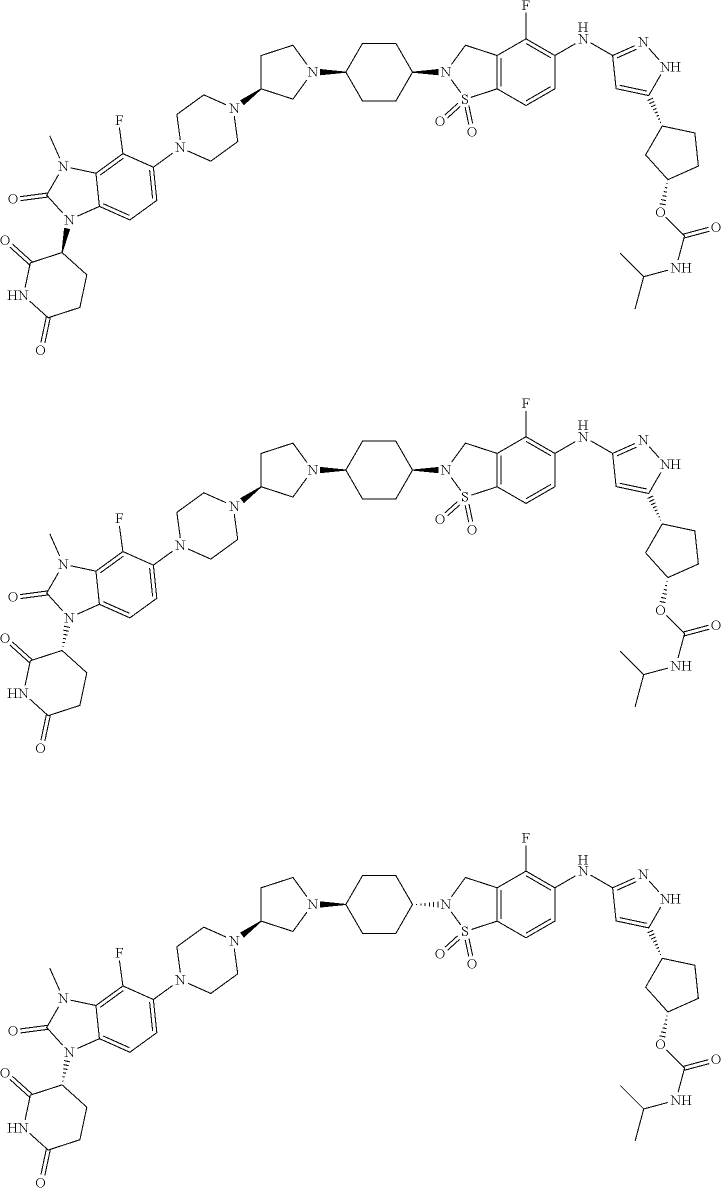 [SMILES: CC(C)NC(=O)O[C@H]1CC[C@@H](c2cc(Nc3ccc4c(c3F)CN([C@H]3CC[C@@H](N5CC[C@H](N6CCN(c7ccc8c(c7F)n(C)c(=O)n8[C@H]7CCC(=O)NC7=O)CC6)C5)CC3)S4(=O)=O)n[nH]2)C1 ; CC(C)NC(=O)O[C@H]1CC[C@@H](c2cc(Nc3ccc4c(c3F)CN([C@H]3CC[C@@H](N5CC[C@H](N6CCN(c7ccc8c(c7F)n(C)c(=O)n8[C@@H]7CCC(=O)NC7=O)CC6)C5)CC3)S4(=O)=O)n[nH]2)C1 ; CC(C)NC(=O)O[C@H]1CC[C@@H](c2cc(Nc3ccc4c(c3F)CN([C@H]3CC[C@H](N5CC[C@H](N6CCN(c7ccc8c(c7F)n(C)c(=O)n8[C@@H]7CCC(=O)NC7=O)CC6)C5)CC3)S4(=O)=O)n[nH]2)C1]

The racemic compound was synthesized in the procedures similar to Example 2, which was further separated by Chiral Prep-HPLC to afford:

Enantiomer 1 (100% ee); Retention time: 6.42 min. ¹H NMR (500 MHz, DMSO) δ 11.98 (s, 1H), 11.10 (s, 1H), 8.76 (s, 1H), 8.30 (s, 1H), 7.50 (d, J=8.8 Hz, 1H), 6.94 (s, 1H), 6.85 (d, J=8.9 Hz, 1H), 6.72 (t, J=8.2 Hz, 1H), 5.82 (s, 1H), 5.33 (dd, J=12.6, 5.3 Hz, 1H), 5.00 (s, 1H), 4.52-4.39 (m, 2H), 3.57 (s, 2H), 3.50-3.45 (m, 4H), 3.06 (s, 2H), 2.96-2.86 (m, 7H), 2.75-2.62 (m, 6H), 2.36 (s, 1H), 2.14 (s, 1H), 2.04-1.99 (m, 4H), 1.95-1.82 (m, 4H), 1.76-1.52 (m, 9H), 1.03 (d, J=5.8 Hz, 6H). LC-MS (ESI): m/z [M+H]⁺=948.58.

Enantiomer 2 (100% ee); Retention time: 10.57 min. ¹H NMR (500 MHz, DMSO) δ 11.99 (s, 1H), 11.10 (s, 1H), 8.76 (s, 1H), 8.29 (s, 1H), 7.50 (d, J=8.6 Hz, 1H), 6.94 (d, J=7.4 Hz, 1H), 6.86 (d, J=8.5 Hz, 1H), 6.72 (t, J=8.2 Hz, 1H), 5.83 (s, 1H), 5.33 (dd, J=12.5, 5.4 Hz, 1H), 4.99 (s, 1H), 4.50 (s, 2H), 3.47 (s, 3H), 3.08-3.02 (m, 2H), 2.95 (s, 4H), 2.89 (s, 3H), 2.81 (s, 2H), 2.69-2.64 (m, 7H), 2.57 (s, 3H), 2.05-1.96 (m, 6H), 1.93-1.89 (m, 2H), 1.71-1.64 (m, 4H), 1.30-1.24 (m, 4H), 1.03-1.02 (d, J=6.1 Hz, 6H). LC-MS (ESI): m/z [M+H]+=948.58.

Enantiomer 3 (100% ee); Retention time: 12.28 min. ¹H NMR (500 MHz, DMSO) δ 11.99 (s, 1H), 11.10 (s, 1H), 8.76 (s, 1H), 8.29 (s, 1H), 7.50 (d, J=8.6 Hz, 1H), 6.94 (d, J=7.4 Hz, 1H), 6.86 (d, J=8.5 Hz, 1H), 6.72 (t, J=8.2 Hz, 1H), 5.83 (s, 1H), 5.33 (dd, J=12.5, 5.4 Hz, 1H), 4.99 (s, 1H), 4.50 (s, 2H), 3.47 (s, 3H), 3.08-3.02 (m, 2H), 2.95 (s, 4H), 2.89 (s, 3H), 2.81 (s, 2H), 2.69-2.64 (m, 7H), 2.57 (s, 3H), 2.05-1.96 (m, 6H), 1.93-1.89 (m, 2H), 1.71-1.64 (m, 4H), 1.30-1.24 (m, 4H), 1.03-1.02 (d, J=6.1 Hz, 6H). LC-MS (ESI): m/z [M+H]+=948.58.

Chiral analytical method: Column: I-Cellulose-5, 4.6 mm×250 mm, 5 um; Mobile phase: A for Hexane and B for EtOH (0.1% 2M NH₃ MeOH); Gradient: Mobile Phase A: Mobile Phase B=30:70 (v/v); HPLC Equipment: HPLC-Agilent, Back pressure: 100 bar; Column temperature: 35° C.

Chiral Prep-HPLC Condition: Column: I-Cellulose-5, 21.2 mm×250 mm, 5 um; Mobile phase: A for Hexane and B for EtOH (0.2% 2M NH₃ MeOH); Gradient: Mobile Phase A: Mobile Phase B=30:70 (v/v); Flow Rate: 20 mL/min, Wave Length: UV 200 nm and 270 nm, Prep-HPLC Equipment: Prep-HPLC-Gilson, Back pressure: 100 bar; Column temperature: 25° C.

### Example 193a and 193b: (1S,3R)-3-(3-((2-((1S,4S)-4-((R)-3-(4-(1-((R)-2,6-dioxopiperidin-3-yl)-4-fluoro-3-methyl-2-oxo-2,3-dihydro-1H-benzo[d]imidazol-5-yl)piperazin-1-yl)pyrrolidin-1-yl)cyclohexyl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-((1R,4R)-4-((R)-3-(4-(1-((R)-2,6-dioxopiperidin-3-yl)-4-fluoro-3-methyl-2-oxo-2,3-dihydro-1H-benzo[d]imidazol-5-yl)piperazin-1-yl)pyrrolidin-1-yl)cyclohexyl)-4-fluoro-1,1-dioxido-2,3-dihydrobenzo[d]isothiazol-5-yl)amino)-1H-pyrazol-5-yl)cyclopentyl isopropylcarbamate

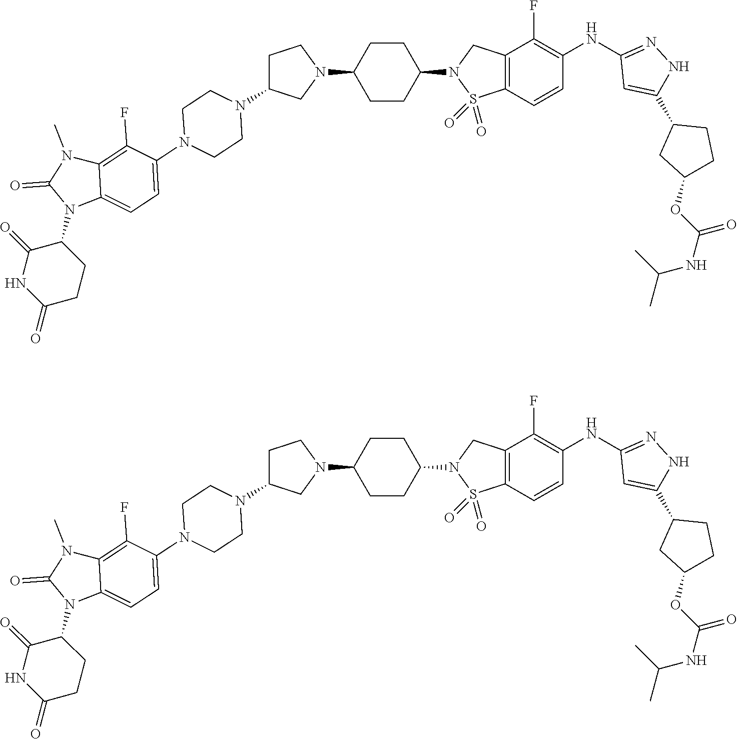 [SMILES: CC(C)NC(=O)O[C@H]1CC[C@@H](c2cc(Nc3ccc4c(c3F)CN([C@H]3CC[C@@H](N5CC[C@@H](N6CCN(c7ccc8c(c7F)n(C)c(=O)n8[C@@H]7CCC(=O)NC7=O)CC6)C5)CC3)S4(=O)=O)n[nH]2)C1 ; CC(C)NC(=O)O[C@H]1CC[C@@H](c2cc(Nc3ccc4c(c3F)CN([C@H]3CC[C@H](N5CC[C@@H](N6CCN(c7ccc8c(c7F)n(C)c(=O)n8[C@@H]7CCC(=O)NC7=O)CC6)C5)CC3)S4(=O)=O)n[nH]2)C1]

### Step 1: tert-butyl (3R)-3-(4-(1-(2,6-bis(benzyloxy)pyridin-3-yl)-4-fluoro-3-methyl-2-oxo-2,3-dihydro-1H-benzo[d]imidazol-5-yl)piperazin-1-yl)pyrrolidine-1-carboxylate

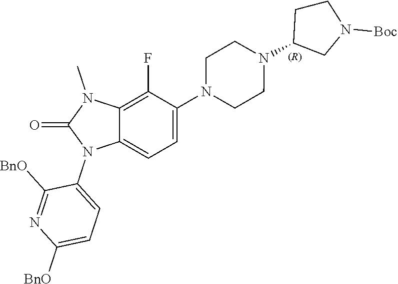

To a mixture of t 1-(2,6-bis(benzyloxy)pyridin-3-yl)-5-bromo-4-fluoro-3-methyl-1,3-dihydro-2H-benzo[d]imidazol-2-one (1 g, 1.87 mmol) and tert-butyl (R)-3-(piperazin-1-yl)pyrrolidine-1-carboxylate (478 mg, 1.87 mmol) in andydrous 1,4-dioxane (20 mL) was added Pd-PEPPSI (182 mg, 0.19 mmol) and Cs₃CO₃ (1.22 g, 3.74 mmol). The reaction mixture was stirred under nitrogen atmosphere at 90° C. for 16 h. The mixture was filtered, and the filtrate was concentrated under reduced pressure. The residue was purified by silica gel column chromatography, eluting with DCM/MeOH (1:20) to afford the product (690 mg, 52% yield). LC-MS (ESI): m/z [M+H]⁺=709.7.

### Step 2: tert-butyl (3R)-3-(4-(1-(2,6-dioxopiperidin-3-yl)-4-fluoro-3-methyl-2-oxo-2,3-dihydro-1H-benzo[d]imidazol-5-yl)piperazin-1-yl)pyrrolidine-1-carboxylate

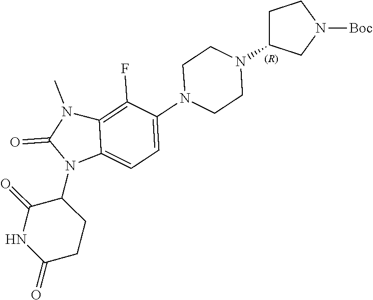

To a solution of tert-butyl (3R)-3-(4-(1-(2,6-bis(benzyloxy)pyridin-3-yl)-4-fluoro-3-methyl-2-oxo-2,3-dihydro-1H-benzo[d]imidazol-5-yl)piperazin-1-yl)pyrrolidine-1-carboxylate (690 mg, 0.8 mmol) in THF (20 mL) was added Pd/C (10%, wet, 500 mg). The resulting mixture was stirred at room temperature under a hydrogen balloon for 16 h. The mixture was filtered, and the filter cake was washed with MeOH (50 mL×3). The filtrate was concentrated under reduced pressure to afford the product (300 mg, 59% yield). LC-MS (ESI): m/z [M+H]⁺=531.5.

### Step 3: 3-(4-fluoro-3-methyl-2-oxo-5-(4-((R)-pyrrolidin-3-yl)piperazin-1-yl)-2,3-dihydro-1H-benzo[d]imidazol-1-yl)piperidine-2,6-dione

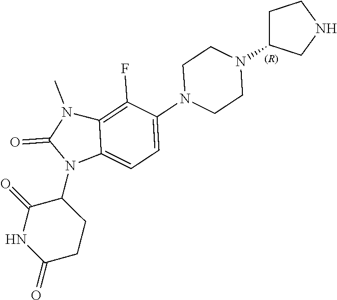

A solution of tert-butyl (3R)-3-(4-(1-(2,6-dioxopiperidin-3-yl)-4-fluoro-3-methyl-2-oxo-2,3-dihydro-1H-benzo[d]imidazol-5-yl)piperazin-1-yl)pyrrolidine-1-carboxylate (300 mg, 0.56 mmol) in DCM (2 mL) was treated with triflic acid (2 mL) dropwise. The resulting mixture was stirred at room temperature for 2 h. The reaction mixture was quenched and basified to pH=8 with sat. aq NaHCO₃ at 0° C. The layers separated and the aqueous layer was extracted with DCM/MeOH=20/1 (50 mL×3). The combined organic layers were washed with brine (50 mL) and dried over anhydrous Na₂SO₄. After filtration, the filtrate was concentrated under reduced pressure to afford the product (200 mg, 82% yield). LC-MS (ESI): m/z [M+H]⁺=431.4.

### Step 4: (1S,3R)-3-(3-((2-((1S,4S)-4-((R)-3-(4-(1-((R)-2,6-dioxopiperidin-3-yl)-4-fluoro-3-methyl-2-oxo-2,3-dihydro-1H-benzo[d]imidazol-5-yl)piperazin-1-yl)pyrrolidin-1-yl)cyclohexyl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-((1R,4R)-4-((R)-3-(4-(1-((R)-2,6-dioxopiperidin-3-yl)-4-fluoro-3-methyl-2-oxo-2,3-dihydro-1H-benzo[d]imidazol-5-yl)piperazin-1-yl)pyrrolidin-1-yl)cyclohexyl)-4-fluoro-1,1-dioxido-2,3-dihydrobenzo[d]isothiazol-5-yl)amino)-1H-pyrazol-5-yl)cyclopentyl isopropylcarbamate

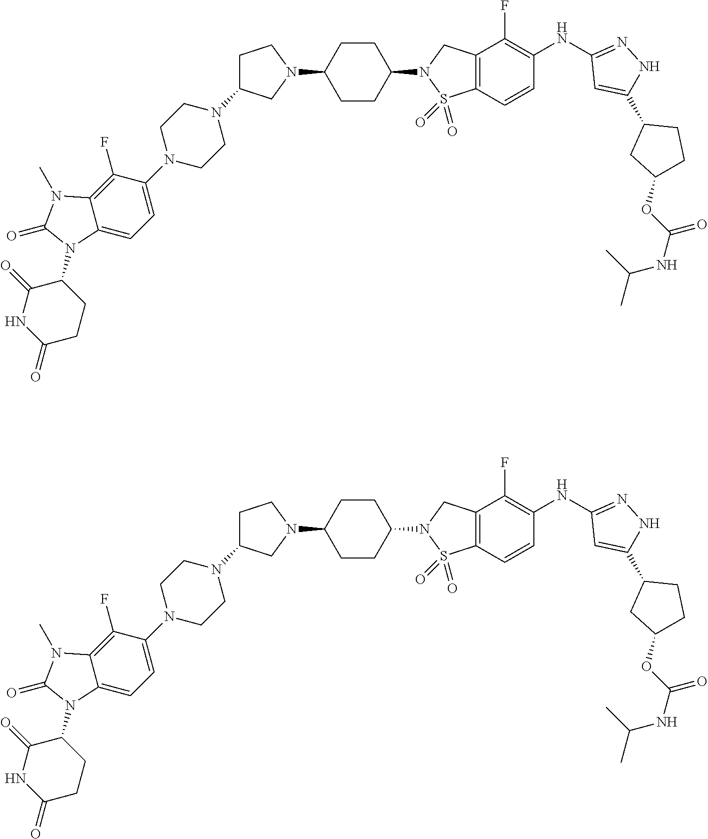 [SMILES: CC(C)NC(=O)O[C@H]1CC[C@@H](c2cc(Nc3ccc4c(c3F)CN([C@H]3CC[C@@H](N5CC[C@@H](N6CCN(c7ccc8c(c7F)n(C)c(=O)n8[C@@H]7CCC(=O)NC7=O)CC6)C5)CC3)S4(=O)=O)n[nH]2)C1 ; CC(C)NC(=O)O[C@H]1CC[C@@H](c2cc(Nc3ccc4c(c3F)CN([C@H]3CC[C@H](N5CC[C@@H](N6CCN(c7ccc8c(c7F)n(C)c(=O)n8[C@@H]7CCC(=O)NC7=O)CC6)C5)CC3)S4(=O)=O)n[nH]2)C1]

The racemic compound was synthesized in the procedures similar to Example 2, which was further separated by Chiral Prep-HPLC to afford:

Enantiomer 1 (100% ee); Retention time: 10.53 min. ¹H NMR (500 MHz, DMSO) δ 11.99 (s, 1H), 11.10 (s, 1H), 8.76 (s, 1H), 8.29 (s, 1H), 7.50 (d, J=8.6 Hz, 1H), 6.94 (d, J=7.4 Hz, 1H), 6.86 (d, J=8.5 Hz, 1H), 6.72 (t, J=8.1 Hz, 1H), 5.83 (s, 1H), 5.33 (dd, J=12.7, 5.5 Hz, 1H), 5.00 (s, 1H), 4.49 (s, 2H), 3.57 (s, 1H), 3.47-3.43 (m, 4H), 3.06 (s, 1H), 2.95 (s, 4H), 2.90-2.79 (m, 3H), 2.73-2.57 (m, 8H), 2.36 (s, 2H), 2.09-1.93 (m, 7H), 1.91-1.89 (dm, 2H), 1.71-1.63 (m, 4H), 1.24 (s, 4H), 1.03-1.02 (m, 6H). LC-MS (ESI): m/z [M+H]+=948.58.

Enantiomer 2 (100% ee); Retention time: 12.51 min. ¹H NMR (500 MHz, DMSO) δ 11.99 (s, 1H), 11.10 (s, 1H), 8.76 (s, 1H), 8.29 (s, 1H), 7.50 (d, J=8.6 Hz, 1H), 6.94 (d, J=7.4 Hz, 1H), 6.86 (d, J=8.5 Hz, 1H), 6.72 (t, J=8.1 Hz, 1H), 5.83 (s, 1H), 5.33 (dd, J=12.7, 5.5 Hz, 1H), 5.00 (s, 1H), 4.49 (s, 2H), 3.57 (s, 1H), 3.47-3.43 (m, 4H), 3.06 (s, 1H), 2.95 (s, 4H), 2.90-2.79 (m, 3H), 2.73-2.57 (m, 8H), 2.36 (s, 2H), 2.09-1.93 (m, 7H), 1.91-1.89 (dm, 2H), 1.71-1.63 (m, 4H), 1.24 (s, 4H), 1.03-1.02 (m, 6H). LC-MS (ESI): m/z [M+H]+=948.58.

Chiral analytical method: Column: I-Cellulose-5, 4.6 mm×250 mm, 5 um; Mobile phase: A for Hexane and B for EtOH (0.1% 2M NH₃ MeOH); Gradient: Mobile Phase A: Mobile Phase B=30:70 (v/v); HPLC Equipment: HPLC-Agilent, Back pressure: 100 bar; Column temperature: 35° C.

Chiral Prep-HPLC Condition: Column: I-Cellulose-5, 21.2 mm×250 mm, 5 um; Mobile phase: A for Hexane and B for EtOH (0.2% 2M NH₃ MeOH); Gradient: Mobile Phase A: Mobile Phase B=30:70 (v/v); Flow Rate: 20 mL/min, Wave Length: UV 200 nm and 270 nm, Prep-HPLC Equipment: Prep-HPLC-Gilson, Back pressure: 100 bar; Column temperature: 25° C.

### Example 194: (1S,3R)-3-(3-((2-(1-(2-(1-(2,6-dioxopiperidin-3-yl)-4-fluoro-3-methyl-2-oxo-2,3-dihydro-1H-benzo[d]imidazol-5-yl)ethyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

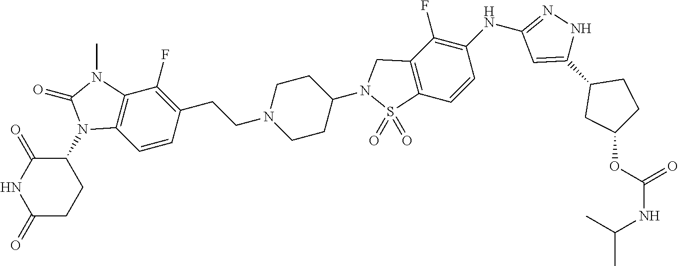 [SMILES: CC(C)NC(=O)O[C@H]1CC[C@@H](c2cc(Nc3ccc4c(c3F)CN(C3CCN(CCc5ccc6c(c5F)n(C)c(=O)n6[C@@H]5CCC(=O)NC5=O)CC3)S4(=O)=O)n[nH]2)C1]

The titled compound was synthesized in the procedures similar to Example 39. LC-MS (ESI): m/z [M+H]⁺=824.48; ¹H NMR (500 MHz, DMSO) δ=12.00 (s, 1H), 11.11 (s, 1H), 8.76 (s, 1H), 8.29 (s, 1H), 7.51 (d, J=8.6, 1H), 7.00-6.86 (m, 3H), 5.83 (s, 1H), 5.36 (dd, J=12.8, 5.3, 1H), 5.00 (s, 1H), 4.51 (s, 2H), 3.49 (m, 4H), 3.06 (m, 1H), 3.00 (d, J=10.8, 2H), 2.88 (m, 2H), 2.79 (m, 2H), 2.74-2.68 (m, 2H), 2.64 (m, 1H), 2.61 (m, 1H), 2.46-2.43 (m, 1H), 2.13 (t, J=10.9, 2H), 2.03 (m, 2H), 1.94-1.83 (m, 5H), 1.71 (m, 2H), 1.60 (m, 1H), 1.03 (m, 6H).

### Example 195a and 195b: (1S,3R)-3-(3-((2-(1-(((S)-1-(1-((R)-2,6-dioxopiperidin-3-yl)-4-fluoro-3-methyl-2-oxo-2,3-dihydro-1H-benzo[d]imidazol-5-yl)-3,3-dimethylpiperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-(1-(((R)-1-(1-((R)-2,6-dioxopiperidin-3-yl)-4-fluoro-3-methyl-2-oxo-2,3-dihydro-1H-benzo[d]imidazol-5-yl)-3,3-dimethylpiperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

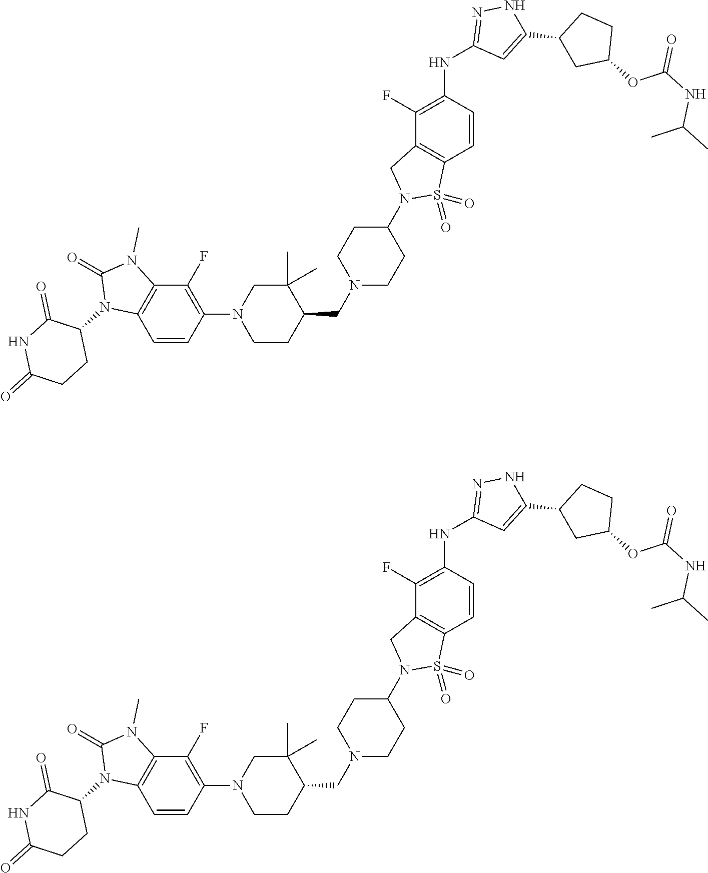 [SMILES: CC(C)NC(=O)O[C@H]1CC[C@@H](c2cc(Nc3ccc4c(c3F)CN(C3CCN(C[C@H]5CCN(c6ccc7c(c6F)n(C)c(=O)n7[C@@H]6CCC(=O)NC6=O)CC5(C)C)CC3)S4(=O)=O)n[nH]2)C1 ; CC(C)NC(=O)O[C@H]1CC[C@@H](c2cc(Nc3ccc4c(c3F)CN(C3CCN(C[C@@H]5CCN(c6ccc7c(c6F)n(C)c(=O)n7[C@@H]6CCC(=O)NC6=O)CC5(C)C)CC3)S4(=O)=O)n[nH]2)C1]

### Step 1: 1-(1-(2,6-dioxopiperidin-3-yl)-4-fluoro-3-methyl-2-oxo-2,3-dihydro-1H-benzo[d]imidazol-5-yl)-3,3-dimethylpiperidine-4-carbaldehyde

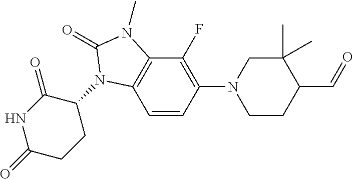 [SMILES: Cn1c(=O)n([C@@H]2CCC(=O)NC2=O)c2ccc(N3CCC(C=O)C(C)(C)C3)c(F)c21]

The racemic compound was synthesized in the procedures similar to Example 120. LC-MS (ESI): m/z [M+H]⁺=417.4.

### Step 2: (1S,3R)-3-(3-((2-(1-(((S)-1-(1-((R)-2,6-dioxopiperidin-3-yl)-4-fluoro-3-methyl-2-oxo-2,3-dihydro-1H-benzo[d]imidazol-5-yl)-3,3-dimethylpiperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-(1-(((R)-1-(1-((R)-2,6-dioxopiperidin-3-yl)-4-fluoro-3-methyl-2-oxo-2,3-dihydro-1H-benzo[d]imidazol-5-yl)-3,3-dimethylpiperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

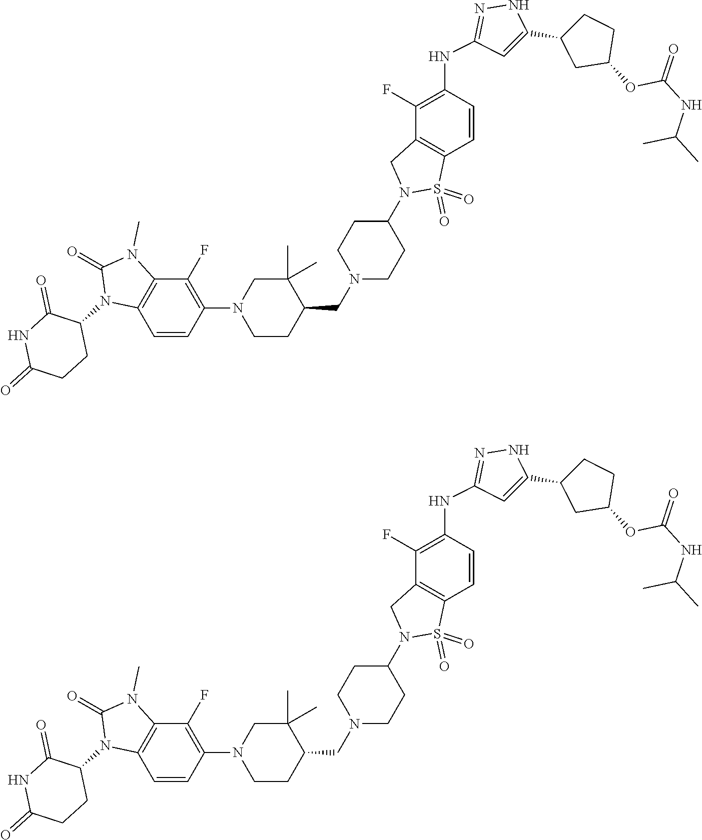 [SMILES: CC(C)NC(=O)O[C@H]1CC[C@@H](c2cc(Nc3ccc4c(c3F)CN(C3CCN(C[C@H]5CCN(c6ccc7c(c6F)n(C)c(=O)n7[C@@H]6CCC(=O)NC6=O)CC5(C)C)CC3)S4(=O)=O)n[nH]2)C1 ; CC(C)NC(=O)O[C@H]1CC[C@@H](c2cc(Nc3ccc4c(c3F)CN(C3CCN(C[C@@H]5CCN(c6ccc7c(c6F)n(C)c(=O)n7[C@@H]6CCC(=O)NC6=O)CC5(C)C)CC3)S4(=O)=O)n[nH]2)C1]

The racemic compound was synthesized in the procedures similar to Example 120, which was further separated by Chiral Prep-HPLC to afford the desired product:

Enantiomer 1 (Example 195a, 99.4% ee); Retention time: 8.16 min. ¹H NMR (500 MHz, DMSO-d₆) δ 11.99 (s, 1H), 11.09 (s, 1H), 8.76 (s, 1H), 8.30 (t, J=10 Hz, 1H), 7.51 (t, J=10 Hz, 1H), 6.98-6.91 (m, 1H), 6.87-6.81 (m, 1H), 6.73-6.67 (m, 1H), 5.83 (s, 1H), 5.37-5.29 (m, 1H), 5.07-4.95 (m, 1H), 4.52 (s, 2H), 3.68-3.53 (m, 1H), 3.47 (s, 3H), 3.45-3.41 (m, 1H), 3.25-3.15 (m, 2H), 3.11-2.95 (m, 2H), 2.94-2.79 (m, 4H), 2.74-2.60 (m, 3H), 2.46-2.40 (m, 1H), 2.40-2.29 (m, 2H), 2.23-1.97 (m, 5H), 1.96-1.80 (m, 7H), 1.78-1.66 (m, 2H), 1.64-1.54 (m, 1H), 1.51-1.32 (m, 2H), 1.09-0.92 (m, 12H). LC-MS (ESI): m/z [M+H]⁺=921.

Enantiomer 2 (Example 195b, 95.7% ee); Retention time: 8.86 min. ¹H NMR (500 MHz, DMSO-d₆) δ 11.99 (s, 1H), 11.10 (s, 1H), 8.77 (s, 1H), 8.30 (t, J=10 Hz, 1H), 7.51 (t, J=10 Hz, 1H), 6.98-6.91 (m, 1H), 6.87-6.81 (m, 1H), 6.73-6.67 (m, 1H), 5.83 (s, 1H), 5.37-5.29 (m, 1H), 5.07-4.95 (m, 1H), 4.52 (s, 2H), 3.68-3.53 (m, 1H), 3.47 (s, 3H), 3.45-3.41 (m, 1H), 3.25-3.15 (m, 2H), 3.11-2.95 (m, 2H), 2.94-2.79 (m, 4H), 2.74-2.60 (m, 3H), 2.46-2.40 (m, 1H), 2.40-2.29 (m, 2H), 2.23-1.97 (m, 5H), 1.96-1.80 (m, 7H), 1.78-1.66 (m, 2H), 1.64-1.54 (m, 1H), 1.51-1.32 (m, 2H), 1.09-0.92 (m, 12H). LC-MS (ESI): m/z [M+H]⁺=921.

Chiral analytical method: Amylose-SA, 4.6 mm×250 mm, 5 um; Mobile phase: A for MtBE (0.1% 2M NH3 MeOH) and B for DCM:MeOH (50:50); Gradient: Mobile Phase A: Mobile Phase B=30:70 (v/v); HPLC Equipment: HPLC-Agilent, Back pressure: 100 bar; Column temperature: 35° C.

Chiral Prep-HPLC Condition: Column: Amylose-SA, 250 mm×20 mm, 5 um; Mobile phase: A for MtBE (0.1% 2M NH3 MeOH) and B for DCM:MeOH (50:50); Gradient: Mobile Phase A: Mobile Phase B=40:60 (v/v); Flow Rate: 20 mL/min, Wave Length: UV 200 nm and 270 nm, Prep-HPLC Equipment: Prep-HPLC-Gilson, Back pressure: 100 bar; Column temperature: 25° C.

### Example 196a and 196b: (1S,3R)-3-(3-((2-((1S,4R)-4-(4-((S)-1-(1-((S)-2,6-dioxopiperidin-3-yl)-4-fluoro-3-methyl-2-oxo-2,3-dihydro-1H-benzo[d]imidazol-5-yl)pyrrolidin-3-yl)piperazin-1-yl)cyclohexyl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-((1S,4R)-4-(4-((S)-1-(1-((R)-2,6-dioxopiperidin-3-yl)-4-fluoro-3-methyl-2-oxo-2,3-dihydro-1H-benzo[d]imidazol-5-yl)pyrrolidin-3-yl)piperazin-1-yl)cyclohexyl)-4-fluoro-1,1-dioxido-2,3-dihydrobenzo[d]isothiazol-5-yl)amino)-1H-pyrazol-5-yl)cyclopentyl isopropylcarbamate

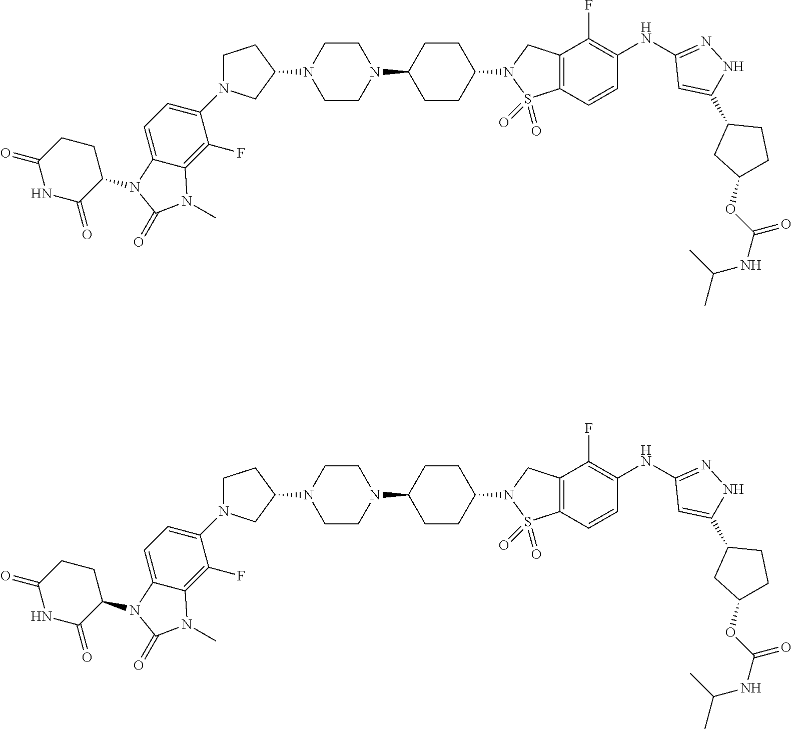 [SMILES: CC(C)NC(=O)O[C@H]1CC[C@@H](c2cc(Nc3ccc4c(c3F)CN([C@H]3CC[C@H](N5CCN([C@H]6CCN(c7ccc8c(c7F)n(C)c(=O)n8[C@H]7CCC(=O)NC7=O)C6)CC5)CC3)S4(=O)=O)n[nH]2)C1 ; CC(C)NC(=O)O[C@H]1CC[C@@H](c2cc(Nc3ccc4c(c3F)CN([C@H]3CC[C@H](N5CCN([C@H]6CCN(c7ccc8c(c7F)n(C)c(=O)n8[C@@H]7CCC(=O)NC7=O)C6)CC5)CC3)S4(=O)=O)n[nH]2)C1]

### Step 1: (S)-3-(4-fluoro-3-methyl-2-oxo-5-((S)-3-(piperazin-1-yl)pyrrolidin-1-yl)-2,3-dihydro-1H-benzo[d]imidazol-1-yl)piperidine-2,6-dione

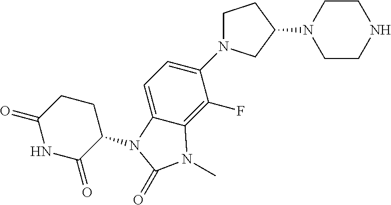 [SMILES: Cn1c(=O)n([C@H]2CCC(=O)NC2=O)c2ccc(N3CC[C@H](N4CCNCC4)C3)c(F)c21]

The racemic compound was synthesized in the procedures similar to Example 192. LC-MS (ESI): m/z [M+H]⁺=431.4.

### Step 2: (1S,3R)-3-(3-((2-((1S,4R)-4-(4-((S)-1-(1-((S)-2,6-dioxopiperidin-3-yl)-4-fluoro-3-methyl-2-oxo-2,3-dihydro-1H-benzo[d]imidazol-5-yl)pyrrolidin-3-yl)piperazin-1-yl)cyclohexyl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-((1S,4R)-4-(4-((S)-1-(1-((R)-2,6-dioxopiperidin-3-yl)-4-fluoro-3-methyl-2-oxo-2,3-dihydro-1H-benzo[d]imidazol-5-yl)pyrrolidin-3-yl)piperazin-1-yl)cyclohexyl)-4-fluoro-1,1-dioxido-2,3-dihydrobenzo[d]isothiazol-5-yl)amino)-1H-pyrazol-5-yl)cyclopentyl isopropylcarbamate

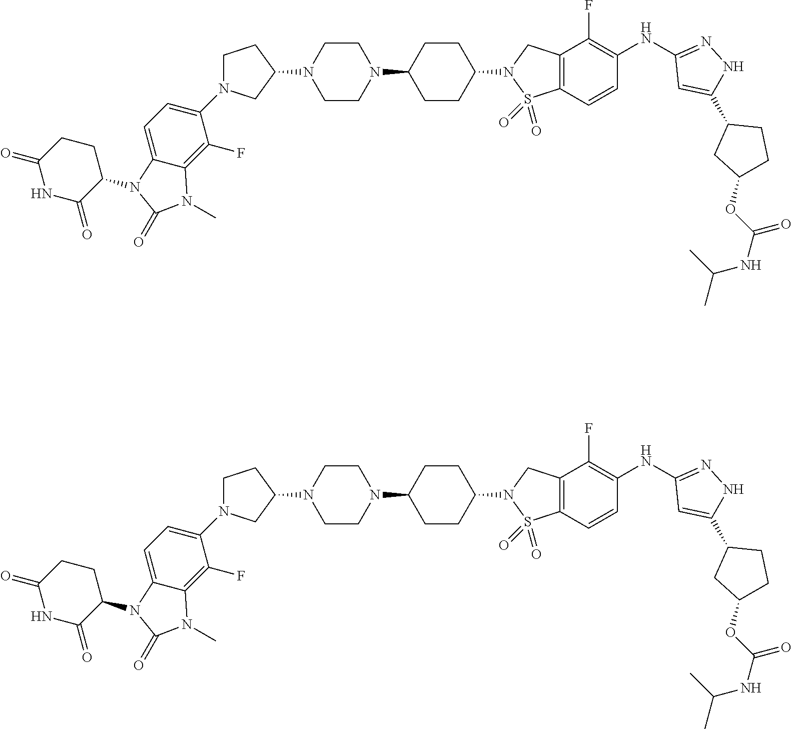 [SMILES: CC(C)NC(=O)O[C@H]1CC[C@@H](c2cc(Nc3ccc4c(c3F)CN([C@H]3CC[C@H](N5CCN([C@H]6CCN(c7ccc8c(c7F)n(C)c(=O)n8[C@H]7CCC(=O)NC7=O)C6)CC5)CC3)S4(=O)=O)n[nH]2)C1 ; CC(C)NC(=O)O[C@H]1CC[C@@H](c2cc(Nc3ccc4c(c3F)CN([C@H]3CC[C@H](N5CCN([C@H]6CCN(c7ccc8c(c7F)n(C)c(=O)n8[C@@H]7CCC(=O)NC7=O)C6)CC5)CC3)S4(=O)=O)n[nH]2)C1]

The racemic compound was synthesized in the procedures similar to Example 192 and separated by chiral HPLC to give:

Enantiomer 1 (Example 196a, 100% ee); Retention time: 8.07 min. ¹H NMR (500 MHz, DMSO) δ 11.98 (s, 1H), 11.08 (s, 1H), 8.75 (s, 1H), 8.29 (s, 1H), 7.50 (d, J=9.0 Hz, 1H), 6.95 (s, 1H), 6.78 (d, J=8.0 Hz, 1H), 6.48 (s, 1H), 5.83 (s, 1H), 5.30 (s, 1H), 5.00 (s, 1H), 4.49 (s, 1H), 3.61-3.51 (m, 1H), 3.46 (s, 4H), 3.17 (s, 1H), 3.06 (s, 1H), 2.85 (s, 3H), 2.64-2.63 (m, 11H), 2.23 (s, 2H), 2.10 (s, 1H), 2.09-1.94 (m, 5H), 1.91 (s, 3H), 1.71-1.67 (m, 4H), 1.37 (s, 2H), 1.31-1.19 (m, 3H), 1.03-1.02 (m, 6H). LC-MS (ESI): m/z [M+H]⁺=948.88.

Enantiomer 2 (Example 196b, 100% ee); Retention time: 14.09 min. ¹H NMR (500 MHz, DMSO) δ 11.98 (s, 1H), 11.08 (s, 1H), 8.75 (s, 1H), 8.29 (s, 1H), 7.50 (d, J=9.0 Hz, 1H), 6.95 (s, 1H), 6.78 (d, J=8.0 Hz, 1H), 6.48 (s, 1H), 5.83 (s, 1H), 5.30 (s, 1H), 5.00 (s, 1H), 4.49 (s, 1H), 3.61-3.51 (m, 1H), 3.46 (s, 4H), 3.17 (s, 1H), 3.06 (s, 1H), 2.85 (s, 3H), 2.64-2.63 (m, 11H), 2.23 (s, 2H), 2.10 (s, 1H), 2.09-1.94 (m, 5H), 1.91 (s, 3H), 1.71-1.67 (m, 4H), 1.37 (s, 2H), 1.31-1.19 (m, 3H), 1.03-1.02 (m, 6H). LC-MS (ESI): m/z [M+H]⁺=948.88.

Chiral analytical method: Column: I-Cellulose-5, 4.6 mm×250 mm, 5 um; Mobile phase: A for Hexane and B for EtOH (0.1% 2M NH₃ MeOH); Gradient: Mobile Phase A: Mobile Phase B=20:80 (v/v); HPLC Equipment: HPLC-Agilent, Back pressure: 100 bar; Column temperature: 35° C.

Chiral Prep-HPLC Condition: Column: I-Cellulose-5, 21.2 mm×250 mm, 5 um; Mobile phase: A for Hexane and B for EtOH (0.2% 2M NH₃ MeOH); Gradient: Mobile Phase A: Mobile Phase B=20:80 (v/v); Flow Rate: 20 mL/min, Wave Length: UV 200 nm and 270 nm, Prep-HPLC Equipment: Prep-HPLC-Gilson, Back pressure: 100 bar; Column temperature: 25° C.

### Example 197: (1S,3R)-3-(3-((2-((1S,4R)-4-((S)-4-(1-((R)-2,6-dioxopiperidin-3-yl)-4-fluoro-3-methyl-2-oxo-2,3-dihydro-1H-benzo[d]imidazol-5-yl)-2-(fluoromethyl)piperazin-1-yl)cyclohexyl)-4-fluoro-1,1-dioxido-2,3-dihydrobenzo[d]isothiazol-5-yl)amino)-1H-pyrazol-5-yl)cyclopentyl isopropylcarbamate

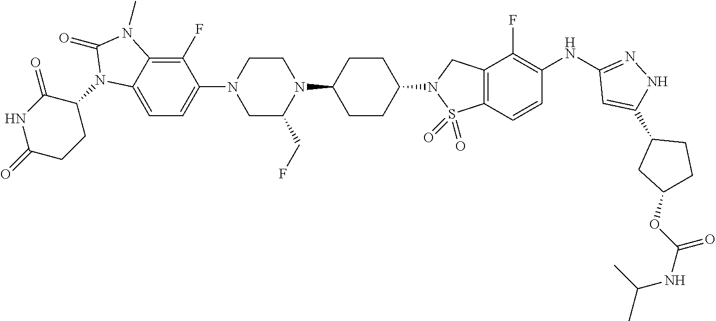 [SMILES: CC(C)NC(=O)O[C@H]1CC[C@@H](c2cc(Nc3ccc4c(c3F)CN([C@H]3CC[C@H](N5CCN(c6ccc7c(c6F)n(C)c(=O)n7[C@@H]6CCC(=O)NC6=O)C[C@H]5CF)CC3)S4(=O)=O)n[nH]2)C1]

### Step 1: tert-butyl (2S)-4-(1-(2,6-bis(benzyloxy)pyridin-3-yl)-4-fluoro-3-methyl-2-oxo-2,3-dihydro-1H-benzo[d]imidazol-5-yl)-2-(fluoromethyl)piperazine-1-carboxylate

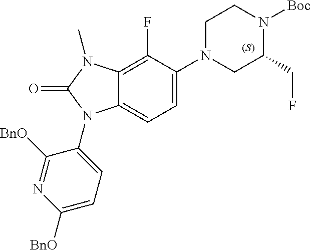

To a mixture of t 1-(2,6-bis(benzyloxy)pyridin-3-yl)-5-bromo-4-fluoro-3-methyl-1,3-dihydro-2H-benzo[d]imidazol-2-one (1 g, 1.87 mmol) and tert-butyl (S)-2-(fluoromethyl)piperazine-1-carboxylate (408 mg, 1.87 mmol) in andydrous 1,4-dioxane (20 mL) was added Pd-PEPPSI (182 mg, 0.19 mmol) and Cs₃CO₃ (1.22 g, 3.74 mmol). The reaction mixture was stirred under nitrogen atmosphere at 90° C. for 16 h. The mixture was filtered, and the filtrate was concentrated under reduced pressure. The residue was purified by silica gel column chromatography, eluting with DCM/MeOH (1:20) to afford the product (820 mg, 65% yield). LC-MS (ESI): m/z [M+H]⁺=672.2.

### Step 2: tert-butyl (2S)-4-(1-(2,6-dioxopiperidin-3-yl)-4-fluoro-3-methyl-2-oxo-2,3-dihydro-1H-benzo[d]imidazol-5-yl)-2-(fluoromethyl)piperazine-1-carboxylate

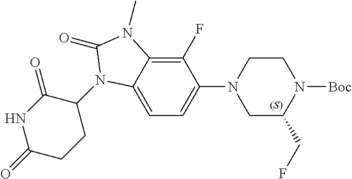

To a solution of tert-butyl (2S)-4-(1-(2,6-bis(benzyloxy)pyridin-3-yl)-4-fluoro-3-methyl-2-oxo-2,3-dihydro-1H-benzo[d]imidazol-5-yl)-2-(fluoromethyl)piperazine-1-carboxylate (820 mg, 1.22 mmol) in THF (20 mL) was added Pd/C (10%, wet, 800 mg). The resulting mixture was stirred at room temperature under a hydrogen balloon for 16 h. The mixture was filtered, and the filter cake was washed with MeOH (50 mL×3). The filtrate was concentrated under reduced pressure to afford the product (430 mg, 71% yield). LC-MS (ESI): m/z [M+H]+=494.5.

### Step 3: 3-(4-fluoro-5-((S)-3-(fluoromethyl)piperazin-1-yl)-3-methyl-2-oxo-2,3-dihydro-1H-benzo[d]imidazol-1-yl)piperidine-2,6-dione

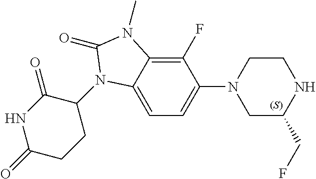

A solution of tert-butyl (2S)-4-(1-(2,6-dioxopiperidin-3-yl)-4-fluoro-3-methyl-2-oxo-2,3-dihydro-1H-benzo[d]imidazol-5-yl)-2-(fluoromethyl)piperazine-1-carboxylate (300 mg, 0.61 mmol) in DCM (2 mL) was treated with triflic acid (2 mL) dropwise. The resulting mixture was stirred at room temperature for 2 h. The reaction mixture was quenched and basified to pH=8 with sat. aq NaHCO₃ at 0° C. The layers separated and the aqueous layer was extracted with DCM/MeOH=20/1 (50 mL×3). The combined organic layers were washed with brine (50 mL) and dried over anhydrous Na₂SO₄. After filtration, the filtrate was concentrated under reduced pressure to afford the product (190 mg, 79% yield). LC-MS (ESI): m/z [M+H]⁺=394.4.

### Step 4: (1S,3R)-3-(3-((2-((1S,4R)-4-((S)-4-(1-((R)-2,6-dioxopiperidin-3-yl)-4-fluoro-3-methyl-2-oxo-2,3-dihydro-1H-benzo[d]imidazol-5-yl)-2-(fluoromethyl)piperazin-1-yl)cyclohexyl)-4-fluoro-1,1-dioxido-2,3-dihydrobenzo[d]isothiazol-5-yl)amino)-1H-pyrazol-5-yl)cyclopentyl isopropylcarbamate

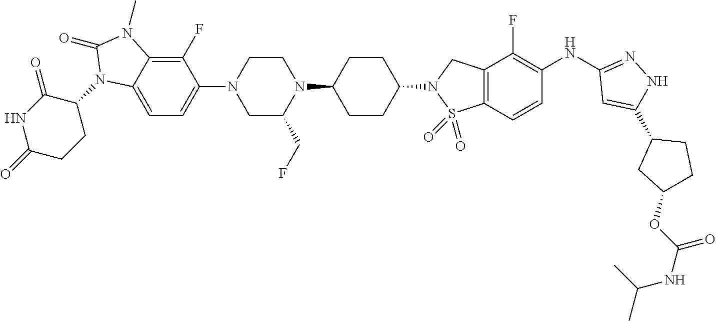 [SMILES: CC(C)NC(=O)O[C@H]1CC[C@@H](c2cc(Nc3ccc4c(c3F)CN([C@H]3CC[C@H](N5CCN(c6ccc7c(c6F)n(C)c(=O)n7[C@@H]6CCC(=O)NC6=O)C[C@H]5CF)CC3)S4(=O)=O)n[nH]2)C1]

The racemic compound was synthesized in the procedures similar to Example 192 and separated by chiral HPLC to give Example 197 (100% ee); Retention time: 13.51 min. ¹H NMR (500 MHz, DMSO) δ 11.98 (s, 1H), 11.08 (s, 1H), 8.75 (s, 1H), 8.29 (s, 1H), 7.50 (d, J=9.0 Hz, 1H), 6.95 (s, 1H), 6.78 (d, J=8.0 Hz, 1H), 6.48 (s, 1H), 5.83 (s, 1H), 5.30 (s, 1H), 5.00 (s, 1H), 4.49 (s, 1H), 3.61-3.51 (m, 1H), 3.46 (s, 4H), 3.17 (s, 1H), 3.06 (s, 1H), 2.85 (s, 3H), 2.64-2.63 (m, 11H), 2.23 (s, 2H), 2.10 (s, 1H), 2.09-1.96 (m, 5H), 1.91 (s, 3H), 1.71-1.67 (m, 4H), 1.37 (s, 2H), 1.24 (s, 3H), 1.03-1.02 (m, 6H). LC-MS (ESI): m/z [M+H]⁺=911.39.

Chiral analytical method: Column: I-Cellulose-5, 4.6 mm×250 mm, 5 um; Mobile phase: A for Hexane and B for EtOH (0.1% 2M NH₃ MeOH); Gradient: Mobile Phase A: Mobile Phase B=50:50 (v/v); HPLC Equipment: HPLC-Agilent, Back pressure: 100 bar; Column temperature: 35° C.

Chiral Prep-HPLC Condition: Column: I-Cellulose-5, 21.2 mm×250 mm, 5 um; Mobile phase: A for Hexane and B for EtOH (0.2% 2M NH₃ MeOH); Gradient: Mobile Phase A: Mobile Phase B=50:50 (v/v); Flow Rate: 20 mL/min, Wave Length: UV 200 nm and 270 nm, Prep-HPLC Equipment: Prep-HPLC-Gilson, Back pressure: 100 bar; Column temperature: 25° C.

### Example 198: (1S,3R)-3-(3-((2-(1-((1-(1-(2,6-dioxopiperidin-3-yl)-4-fluoro-3-methyl-2-oxo-2,3-dihydro-1H-benzo[d]imidazol-5-yl)azetidin-3-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

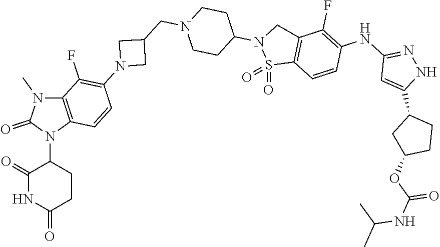 [SMILES: CC(C)NC(=O)O[C@H]1CC[C@@H](c2cc(Nc3ccc4c(c3F)CN(C3CCN(CC5CN(c6ccc7c(c6F)n(C)c(=O)n7C6CCC(=O)NC6=O)C5)CC3)S4(=O)=O)n[nH]2)C1]

### Step 1: 1-(1-(2,6-dioxopiperidin-3-yl)-4-fluoro-3-methyl-2-oxo-2,3-dihydro-H-benzo[d]imidazol-5-yl)azetidine-3-carbaldehyde

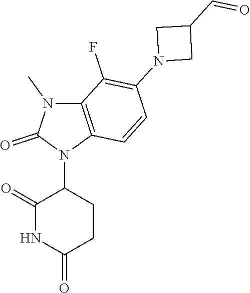 [SMILES: Cn1c(=O)n(C2CCC(=O)NC2=O)c2ccc(N3CC(C=O)C3)c(F)c21]

The titled compound was synthesized in the procedures similar to Example 44. LC-MS (ESI): m/z [M+H]⁺=361.4

### Step 2: (1S,3R)-3-(3-((2-(1-((1-(1-(2,6-dioxopiperidin-3-yl)-4-fluoro-3-methyl-2-oxo-2,3-dihydro-1H-benzo[d]imidazol-5-yl)azetidin-3-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

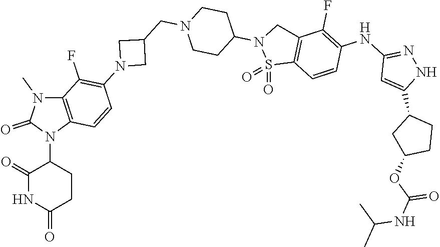 [SMILES: CC(C)NC(=O)O[C@H]1CC[C@@H](c2cc(Nc3ccc4c(c3F)CN(C3CCN(CC5CN(c6ccc7c(c6F)n(C)c(=O)n7C6CCC(=O)NC6=O)C5)CC3)S4(=O)=O)n[nH]2)C1]

The titled compound was synthesized in the procedures similar to Example 36. LC-MS (ESI): m/z [M+H]⁺=865.40; ¹H NMR (500 MHz, DMSO) δ=11.99 (s, 1H), 11.08 (s, 1H), 8.77 (s, 1H), 8.29 (s, 1H), 7.51 (d, J=8.7, 1H), 6.94 (d, J=7.7, 1H), 6.79 (d, J=8.3, 1H), 6.22 (t, J=8.4, 1H), 5.83 (s, 1H), 5.29 (dd, J=12.8, 5.2, 1H), 5.00 (s, 1H), 4.51 (s, 2H), 3.99 (t, J=6.6, 2H), 3.57 (m, 1H), 3.52 (m, 2H), 3.44 (s, 3H), 3.09-3.04 (m, 1H), 2.92 (m, 2H), 2.85 (m, 1H), 2.67 (m, 1H), 2.64 (m, 4H), 2.59 (m, 2H), 2.47-2.43 (m, 1H), 2.15 (m, 1H), 2.04-1.97 (m, 2H), 1.90 (m, 5H), 1.71 (m, 2H), 1.60 (m, 1H), 1.03 (m, 6H).

### Example 199: (1S,3R)-3-(3-((2-(1-(1-(1-(2,6-dioxopiperidin-3-yl)-4-fluoro-3-methyl-2-oxo-2,3-dihydro-1H-benzo[d]imidazol-5-yl)azetidin-3-yl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

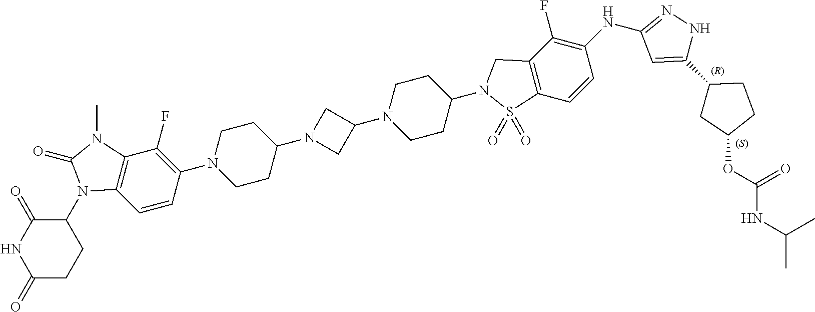

The titled compound was synthesized in the procedures similar to Example 44. ¹H NMR (500 MHz, DMSO) δ 11.99 (s, 1H), 11.08 (s, 1H), 8.76 (s, 1H), 8.28 (s, 1H), 7.51 (d, J=8.6 Hz, 1H), 6.94 (d, J=7.6 Hz, 1H), 6.80 (d, J=8.2 Hz, 1H), 6.24 (t, J=8.4 Hz, 1H), 5.83 (s, 1H), 5.30 (q, J=12.6, 4.9 Hz, 1H), 4.99 (s, 1H), 4.51 (s, 2H), 4.00 (s, 2H), 3.63-3.55 (m, 3H), 3.44 (s, 4H), 3.12-3.03 (m, 1H), 2.97-2.76 (m, 3H), 2.73-2.57 (m, 4H), 2.05-1.84 (m, 9H), 1.71 (s, 2H), 1.59 (s, 1H), 1.03 (d, J=5.8 Hz, 6H). LC-MS (ESI): m/z [M+H]⁺=851.58.

### Example 200a and 200b: (1S,3R)-3-(3-((2-(1-(((R)-1-(1-((S)-2,6-dioxopiperidin-3-yl)-7-fluoro-3-isopropyl-2-oxo-2,3-dihydro-1H-benzo[d]imidazol-5-yl)-3,3-difluoropiperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-(1-(((S)-1-(1-((S)-2,6-dioxopiperidin-3-yl)-7-fluoro-3-isopropyl-2-oxo-2,3-dihydro-1H-benzo[d]imidazol-5-yl)-3,3-difluoropiperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

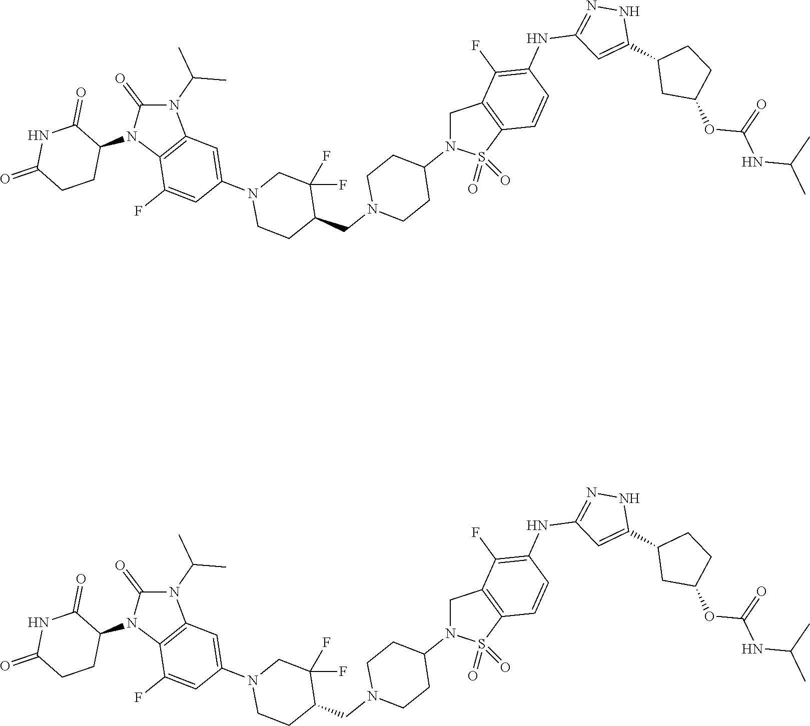 [SMILES: CC(C)NC(=O)O[C@H]1CC[C@@H](c2cc(Nc3ccc4c(c3F)CN(C3CCN(C[C@H]5CCN(c6cc(F)c7c(c6)n(C(C)C)c(=O)n7[C@H]6CCC(=O)NC6=O)CC5(F)F)CC3)S4(=O)=O)n[nH]2)C1 ; CC(C)NC(=O)O[C@H]1CC[C@@H](c2cc(Nc3ccc4c(c3F)CN(C3CCN(C[C@@H]5CCN(c6cc(F)c7c(c6)n(C(C)C)c(=O)n7[C@H]6CCC(=O)NC6=O)CC5(F)F)CC3)S4(=O)=O)n[nH]2)C1]

### Step 1: 1-(1-(2,6-dioxopiperidin-3-yl)-7-fluoro-3-isopropyl-2-oxo-2,3-dihydro-1H-benzo[d]imidazol-5-yl)-3,3-difluoropiperidine-4-carbaldehyde

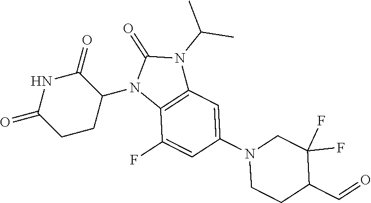 [SMILES: CC(C)n1c(=O)n(C2CCC(=O)NC2=O)c2c(F)cc(N3CCC(C=O)C(F)(F)C3)cc21]

To a solution of 3-(5-(3,3-difluoro-4-(hydroxymethyl)piperidin-1-yl)-7-fluoro-3-isopropyl-2-oxo-2,3-dihydro-1H-benzo[d]imidazol-1-yl)piperidine-2,6-dione (100 mg, 0.22 mmol) in dichloromethane (10 mL) was added Dess-Martin periodinane (140 mg, 0.33 mmol), the reaction mixture was stirred for 2 hour at room temperature. The resulting mixture was concentrated under reduce pressure. The residue was purified by silica gel column chromatography, eluting with PE/EA (1:3) to afford the product (80 mg, 80% yield). LC-MS (ESI): m/z [M+H]⁺=453.2.

### Step 2: (1S,3R)-3-(3-((2-(1-(((R)-1-(1-((S)-2,6-dioxopiperidin-3-yl)-7-fluoro-3-isopropyl-2-oxo-2,3-dihydro-1H-benzo[d]imidazol-5-yl)-3,3-difluoropiperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-(1-(((S)-1-(1-((S)-2,6-dioxopiperidin-3-yl)-7-fluoro-3-isopropyl-2-oxo-2,3-dihydro-1H-benzo[d]imidazol-5-yl)-3,3-difluoropiperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

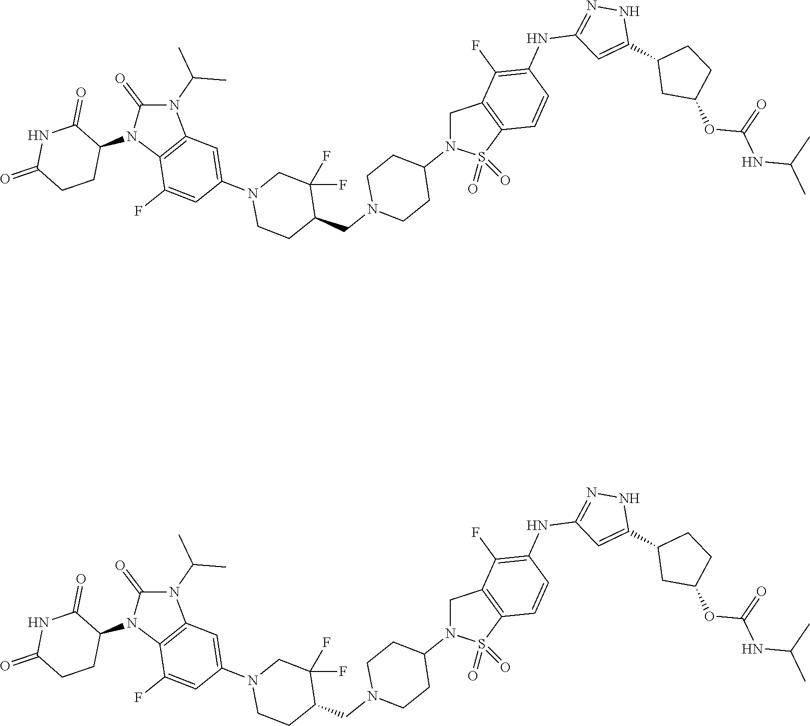 [SMILES: CC(C)NC(=O)O[C@H]1CC[C@@H](c2cc(Nc3ccc4c(c3F)CN(C3CCN(C[C@H]5CCN(c6cc(F)c7c(c6)n(C(C)C)c(=O)n7[C@H]6CCC(=O)NC6=O)CC5(F)F)CC3)S4(=O)=O)n[nH]2)C1 ; CC(C)NC(=O)O[C@H]1CC[C@@H](c2cc(Nc3ccc4c(c3F)CN(C3CCN(C[C@@H]5CCN(c6cc(F)c7c(c6)n(C(C)C)c(=O)n7[C@H]6CCC(=O)NC6=O)CC5(F)F)CC3)S4(=O)=O)n[nH]2)C1]

The titled compound was synthesized in the procedures similar to Example 36 in a racemic form, which was further separated by chiral HPLC to give:

Enantiomer 1 (Example 200a, 100% ee); Retention time: 12.075 min. ¹H NMR (500 MHz, DMSO) δ 11.99 (s, 1H), 11.08 (s, 1H), 8.77 (s, 1H), 8.29 (d, J=7.9 Hz, 1H), 7.51 (d, J=8.6 Hz, 1H), 6.94 (d, J=7.2 Hz, 1H), 6.81 (s, 1H), 6.60 (d, J=15.6 Hz, 1H), 5.83 (s, 1H), 5.45-5.35 (m, 1H), 5.10 4.95 (m, 1H), 4.70-4.60 (m, 1H), 4.52 (s, 2H), 3.92-3.83 (m, 1H), 3.70-3.59 (m, 1H), 3.63-3.53 (m, 1H), 3.50-3.43 (m, 2H), 3.13-2.92 (m, 4H), 2.89-2.82 (m, 2H), 2.75-2.65 (m, 4H), 2.43-2.41 (m, 1H), 2.30-2.15 (m, 3H), 2.13-2.00 (m, 3H), 1.99-1.80 (m, 6H), 1.79-1.65 (m, 2H), 1.63-1.53 (m, 2H), 1.47 (d, J=6.3 Hz, 6H), 1.03 (d, J=6.2 Hz, 6H). LC-MS (ESI): m/z [M+H]⁺=957.4;

Enantiomer 1 (Example 200b, 100% ee); Retention time: 18.212 min. ¹H NMR (500 MHz, DMSO) δ 11.99 (s, 1H), 11.08 (s, 1H), 8.77 (s, 1H), 8.29 (d, J=7.9 Hz, 1H), 7.51 (d, J=8.6 Hz, 1H), 6.94 (d, J=7.2 Hz, 1H), 6.81 (s, 1H), 6.60 (d, J=15.6 Hz, 1H), 5.83 (s, 1H), 5.45-5.35 (m, 1H), 5.10 4.95 (m, 1H), 4.70-4.60 (m, 1H), 4.52 (s, 2H), 3.92-3.83 (m, 1H), 3.70-3.59 (m, 1H), 3.63-3.53 (m, 1H), 3.50-3.43 (m, 2H), 3.13-2.92 (m, 4H), 2.89-2.82 (m, 2H), 2.75-2.65 (m, 4H), 2.43-2.41 (m, 1H), 2.30-2.15 (m, 3H), 2.13-2.00 (m, 3H), 1.99-1.80 (m, 6H), 1.79-1.65 (m, 2H), 1.63-1.53 (m, 2H), 1.47 (d, J=6.3 Hz, 6H), 1.03 (d, J=6.2 Hz, 6H). LC-MS (ESI): m/z [M+H]⁺=957.4;

Chiral analytical method: Column: ICHIRALPAK SA 4.6*150 mm 5 m; Mobile phase: A for MtBE (0.1%2M NH3 MeOH) (%) and B for MeOH:DCM=50:50(%); Gradient: Mobile Phase A: Mobile Phase B=50:50 (v/v); HPLC Equipment: HPLC-Agilent, Back pressure: 100 bar; Column temperature: 35° C.

Chiral Prep-HPLC Condition: CHIRALPAK SA 250*20.0 mml, 5 μm; Mobile phase: A for MtBE (0.1%2M NH3 MeOH) (%) and B for MeOH:DCM=50:50; Gradient: Mobile Phase A: Mobile Phase B=50:50 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 201: (1S,3R)-3-(3-((2-(1′-(1-(2,6-dioxopiperidin-3-yl)-4-fluoro-3-methyl-2-oxo-2,3-dihydro-1H-benzo[d]imidazol-5-yl)-4-methyl-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

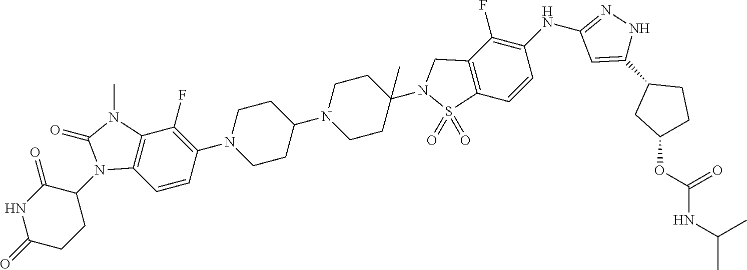 [SMILES: CC(C)NC(=O)O[C@H]1CC[C@@H](c2cc(Nc3ccc4c(c3F)CN(C3(C)CCN(C5CCN(c6ccc7c(c6F)n(C)c(=O)n7C6CCC(=O)NC6=O)CC5)CC3)S4(=O)=O)n[nH]2)C1]

### Step 1: (1S,3R)-3-(3-((4-fluoro-2-(4-methylpiperidin-4-yl)-1,1-dioxido-2,3-dihydrobenzo[d]isothiazol-5-yl)amino)-1H-pyrazol-5-yl)cyclopentyl isopropylcarbamate

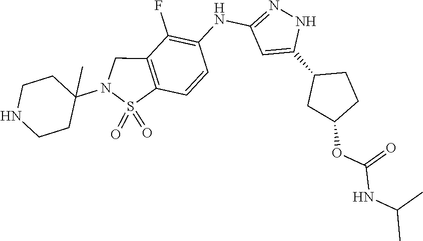 [SMILES: CC(C)NC(=O)O[C@H]1CC[C@@H](c2cc(Nc3ccc4c(c3F)CN(C3(C)CCNCC3)S4(=O)=O)n[nH]2)C1]

The titled compound was synthesized in the procedures similar to Example 1. LC-MS (ESI): m/z [M+H]⁺=535.4;

### Step 2: (1S,3R)-3-(3-((2-(1′-(1-(2,6-dioxopiperidin-3-yl)-4-fluoro-3-methyl-2-oxo-2,3-dihydro-1H-benzo[d]imidazol-5-yl)-4-methyl-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

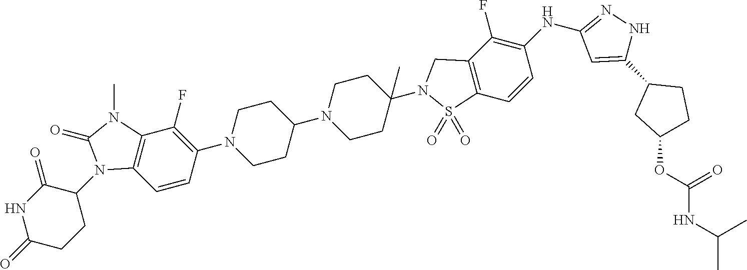 [SMILES: CC(C)NC(=O)O[C@H]1CC[C@@H](c2cc(Nc3ccc4c(c3F)CN(C3(C)CCN(C5CCN(c6ccc7c(c6F)n(C)c(=O)n7C6CCC(=O)NC6=O)CC5)CC3)S4(=O)=O)n[nH]2)C1]

The titled compound was synthesized in the procedures similar to Example 45. ¹H NMR (500 MHz, DMSO) δ 11.99 (s, 1H), 11.10 (s, 1H), 8.76 (s, 1H), 8.30 (t, J=8.0 Hz, 1H), 7.51 (d, J=8.6 Hz, 1H), 6.94 (d, J=7.3 Hz, 1H), 6.40 (d, J=13.6 Hz, 1H), 5.83 (s, 1H), 5.38 (s, 1H), 5.06-4.96 (m, 1H), 4.51 (s, 2H), 4.40-4.32 (m, 2H), 3.98-3.86 (m, 2H), 3.72-3.54 (m, 1H), 3.45-3.37 (m, 3H), 3.12-2.85 (m, 4H), 2.61-2.54 (m, 6H), 2.46-2.42 (m, 1H), 2.13-2.16 (m, 2H), 2.14-2.08 (m, 1H), 2.03 (s, 3H), 1.92-1.82 (m, 5H), 1.81-1.66 (m, 4H), 1.64-1.53 (m, 2H), 1.32-1.19 (m, 2H), 1.03 (d, J=6.3 Hz, 6H). LC-MS (ESI): m/z [M+H]⁺=893.4;

### Example 202: (1S,3R)-3-(3-((2-((3R,4R)-1-((1-(1-((S)-2,6-dioxopiperidin-3-yl)-7-fluoro-3-methyl-2-oxo-2,3-dihydro-1H-benzo[d]imidazol-5-yl)piperidin-4-yl)methyl)-3-fluoropiperidin-4-yl)-4-fluoro-1,1-dioxido-2,3-dihydrobenzo[d]isothiazol-5-yl)amino)-1H-pyrazol-5-yl)cyclopentyl isopropyl carbamate

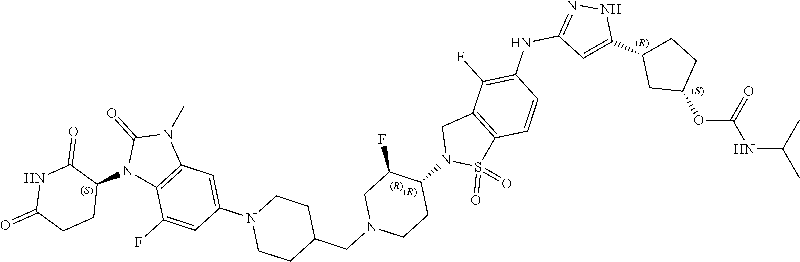

### Step 1: (1S,3R)-3-(3-((4-fluoro-2-((3R,4R)-3-fluoropiperidin-4-yl)-1,1-dioxido-2,3-dihydrobenzo[d]isothiazol-5-yl)amino)-1H-pyrazol-5-yl)cyclopentyl isopropylcarbamate

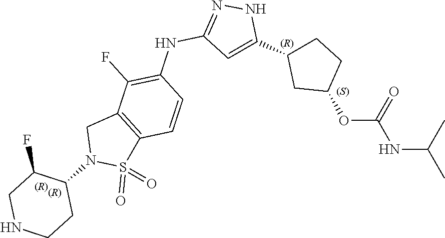

The titled compound was synthesized in the procedures similar to Example 210. LC-MS (ESI): m/z [M+H]⁺=539.5.

### Step 2: (1S,3R)-3-(3-((2-((3R,4R)-1-((1-(1-((S)-2,6-dioxopiperidin-3-yl)-7-fluoro-3-methyl-2-oxo-2,3-dihydro-1H-benzo[d]imidazol-5-yl)piperidin-4-yl)methyl)-3-fluoropiperidin-4-yl)-4-fluoro-1,1-dioxido-2,3-dihydrobenzo[d]isothiazol-5-yl)amino)-1H-pyrazol-5-yl)cyclopentyl isopropyl carbamate

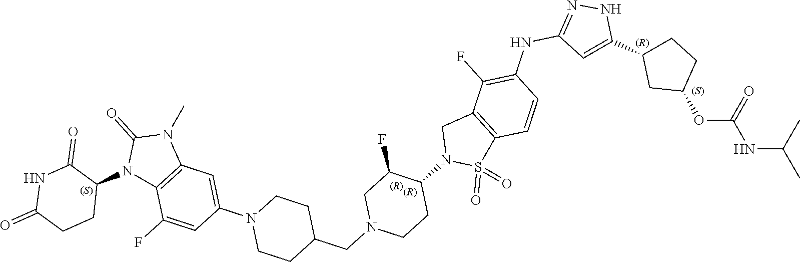

The titled compound was synthesized in the procedures similar to Example 41 in a racemic form, which was further separated by chiral HPLC to give:

Example 202 (100% ee); Retention time: 6.406 min. ¹H NMR (500 MHz, DMSO) δ 12.00 (s, 1H), 11.07 (s, 1H), 8.78 (s, 1H), 8.31 (t, J=7.6 Hz, 1H), 7.54 (d, J=8.6 Hz, 1H), 6.94 (d, J=7.1 Hz, 1H), 6.68 (s, 1H), 6.52 (d, J=14.6 Hz, 1H), 5.84 (s, 1H), 5.41 (s, 1H), 5.00 (s, 1H), 4.90-4.72 (m, 1H), 4.68-4.58 (m, 2H), 3.74-3.55 (m, 4H), 3.34 (s, 3H), 3.26 (m, 1H), 3.11-2.92 (m, 2H), 2.85 (d, J=10.2 Hz, 1H), 2.70-2.60 (m, 4H), 2.32-2.19 (m, 3H), 2.14-1.88 (m, 7H), 1.82-1.59 (m, 6H), 1.29-1.17 (m, 2H), 1.03 (d, J=6.2 Hz, 6H). LC-MS (ESI): m/z [M+H]⁺=911.81.

Chiral analytical method: Column: i-Cellulose-5 4.6*250 mm 5 μm Mobile phase: A for MtBE (0.1%2M NH3 MeOH)(%) and B for MeOH:DCM=50:50 (v/v); Gradient: Mobile Phase A: Mobile Phase B=10:90 (v/v); HPLC Equipment: Agilent 1260 Infinity II (ARD_LC-808); Column temperature: 25° C.

Chiral Prep-HPLC Condition: i-Cellulose-5 4.6*250 mm 5 m Mobile phase: A for MtBE (0.1%2M NH3 MeOH)(%) and B for MeOH:DCM=50:50; Gradient: Mobile Phase A: Mobile Phase B=10:90 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 203: (1S,3R)-3-(3-((2-((3S,4S)-1-((1-(1-((S)-2,6-dioxopiperidin-3-yl)-7-fluoro-3-methyl-2-oxo-2,3-dihydro-1H-benzo[d]imidazol-5-yl)piperidin-4-yl)methyl)-3-fluoropiperidin-4-yl)-4-fluoro-1,1-dioxido-2,3-dihydrobenzo[d]isothiazol-5-yl)amino)-1H-pyrazol-5-yl)cyclopentyl isopropyl carbamate

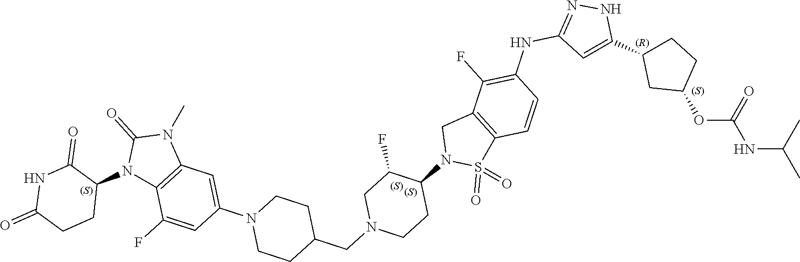

The titled compound was synthesized in the procedures similar to Example 202 in a racemic form, which was further separated by chiral HPLC to give:

Example 203 (100% ee); Retention time: 6.034 min. ¹H NMR (500 MHz, DMSO) δ 12.00 (s, 1H), 11.07 (s, 1H), 8.79 (s, 1H), 8.31 (t, J=7.8 Hz, 1H), 7.54 (d, J=8.6 Hz, 1H), 6.94 (d, J=7.2 Hz, 1H), 6.68 (s, 1H), 6.52 (d, J=14.3 Hz, 1H), 5.84 (s, 1H), 5.41 (s, 1H), 5.00 (s, 1H), 4.90-4.72 (m, 1H), 4.69-4.57 (m, 2H), 3.72-3.52 (m, 4H), 3.34 (s, 3H), 3.28-3.20 (m, 1H), 3.13-2.94 (m, 2H), 2.85 (d, J=9.8 Hz, 1H), 2.72-2.58 (m, 4H), 2.40-2.17 (m, 3H), 2.15-1.88 (m, 7H), 1.82-1.56 (m, 6H), 1.22 (dd, J=22.4, 10.3 Hz, 2H), 1.07-0.99 (m, 6H). LC-MS (ESI): m/z [M+H]⁺=912.36.

Chiral analytical method: Column: i-Cellulose-5 4.6*250 mm 5 μm Mobile phase: A for MtBE (0.1%2M NH3 MeOH)(%) and B for MeOH:DCM=50:50 (v/v); Gradient: Mobile Phase A: Mobile Phase B=10:90 (v/v); HPLC Equipment: Agilent 1260 Infinity II (ARD_LC-808); Column temperature: 25° C.

Chiral Prep-HPLC Condition: i-Cellulose-5 4.6*250 mm 5 m Mobile phase: A for MtBE (0.1%2M NH3 MeOH)(%) and B for MeOH:DCM=50:50; Gradient: Mobile Phase A: Mobile Phase B=10:90 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 204: (1S,3R)-3-(3-((2-((3R,4S)-1-((1-(1-((S)-2,6-dioxopiperidin-3-yl)-7-fluoro-3-methyl-2-oxo-2,3-dihydro-1H-benzo[d]imidazol-5-yl)piperidin-4-yl)methyl)-3-fluoropiperidin-4-yl)-4-fluoro-1,1-dioxido-2,3-dihydrobenzo[d]isothiazol-5-yl)amino)-1H-pyrazol-5-yl)cyclopentyl isopropyl carbamate

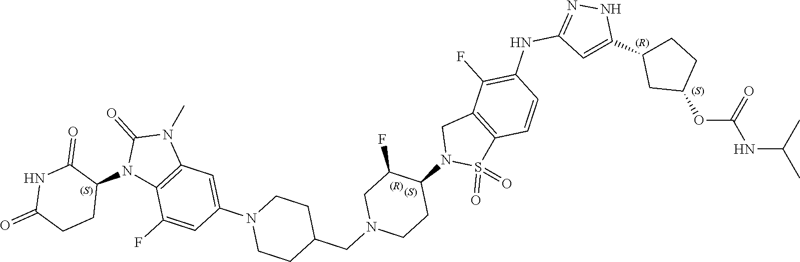

The titled compound was synthesized in the procedures similar to Example 202 in a racemic form, which was further separated by chiral HPLC to give:

Example 204 (100% ee); Retention time: 5.866 min. ¹H NMR (500 MHz, DMSO) δ 12.00 (s, 1H), 11.07 (s, 1H), 8.80 (s, 1H), 8.31 (t, J=7.8 Hz, 1H), 7.56 (d, J=8.7 Hz, 1H), 6.94 (d, J=7.5 Hz, 1H), 6.68 (s, 1H), 6.52 (d, J=14.4 Hz, 1H), 5.84 (s, 1H), 5.41 (s, 1H), 4.96 (d, J=48.3 Hz, 2H), 4.60 (dd, J=48.5, 14.0 Hz, 2H), 3.78-3.53 (m, 4H), 3.34 (s, 3H), 3.1-2.92 (m, 4H), 2.70-2.59 (m, 4H), 2.30-2.14 (m, 6H), 2.08-1.88 (m, 3H), 1.79-1.60 (m, 6H), 1.22 (d, J=12.6 Hz, 2H), 1.06-0.99 (m, 6H). LC-MS (ESI): m/z [M+H]⁺=911.30.

Chiral analytical method: Column: CHIRALPAK ID 4.6*150 mm 5 m; Mobile phase: A for MtBE (0.1%2M NH3 MeOH)(%) and B for MeOH:DCM=50:50 (v/v); Gradient: Mobile Phase A: Mobile Phase B=10:90 (v/v); HPLC Equipment: Agilent 1260 Infinity II (ARD_LC-808); Column temperature: 25° C.

Chiral Prep-HPLC Condition: CHIRALPAK ID 4.6*150 mm 5 m; Mobile phase: A for MtBE (0.1%2M NH3 MeOH)(%) and B for MeOH:DCM=50:50; Gradient: Mobile Phase A: Mobile Phase B=10:90 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 205: (1S,3R)-3-(3-((2-((3S,4R)-1-((1-(1-((S)-2,6-dioxopiperidin-3-yl)-7-fluoro-3-methyl-2-oxo-2,3-dihydro-1H-benzo[d]imidazol-5-yl)piperidin-4-yl)methyl)-3-fluoropiperidin-4-yl)-4-fluoro-1,1-dioxido-2,3-dihydrobenzo[d]isothiazol-5-yl)amino)-1H-pyrazol-5-yl)cyclopentyl isopropyl carbamate

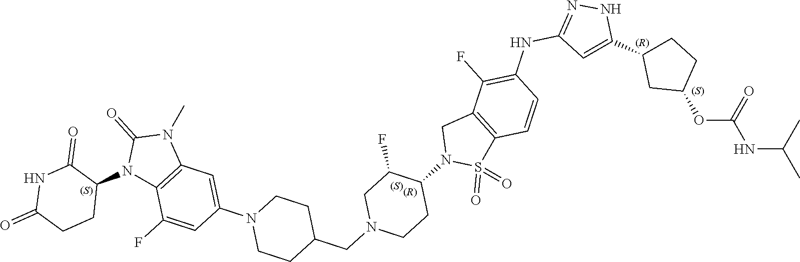

The titled compound was synthesized in the procedures similar to Example 202 in a racemic form, which was further separated by chiral HPLC to give:

Example 205 (100% ee); Retention time: 5.717 min. ¹H NMR (500 MHz, DMSO) δ 12.01 (s, 1H), 11.07 (s, 1H), 8.80 (s, 1H), 8.31 (t, J=7.8 Hz, 1H), 7.57 (t, J=8.4 Hz, 1H), 6.95 (s, 1H), 6.68 (s, 1H), 6.52 (s, 1H), 5.84 (s, 1H), 5.41 (s, 1H), 4.96 (d, J=46.2 Hz, 2H), 4.72-4.48 (m, 2H), 3.80-3.58 (d, 4H), 3.30-3.25 (m, 3H), 3.20-2.92 (m, 4H), 2.74-2.62 (m, 4H), 2.38-2.02 (m, 8H), 1.95-1.60 (m, 8H), 1.22 (s, 2H), 1.08-0.98 (m, 6H). LC-MS (ESI): m/z [M+H]⁺=911.57.

Chiral analytical method: Column: i-Cellulose-5 4.6*250 mm 5 μm Mobile phase: A for MtBE (0.1%2M NH3 MeOH)(%) and B for MeOH:DCM=50:50 (v/v); Gradient: Mobile Phase A: Mobile Phase B=10:90 (v/v); HPLC Equipment: Agilent 1260 Infinity II (ARD_LC-808); Column temperature: 25° C.

Chiral Prep-HPLC Condition: i-Cellulose-5 4.6*250 mm 5 m Mobile phase: A for MtBE (0.1%2M NH3 MeOH)(%) and B for MeOH:DCM=50:50; Gradient: Mobile Phase A: Mobile Phase B=10:90 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 206: (1S,3R)-3-(3-((2-((3R,4R)-1′-(1-((S)-2,6-dioxopiperidin-3-yl)-7-fluoro-3-methyl-2-oxo-2,3-dihydro-1H-benzo[d]imidazol-5-yl)-3-fluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropyl carbamate

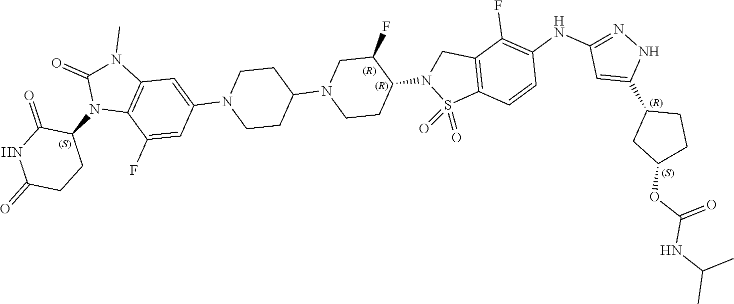

The titled compound was synthesized in the procedures similar to Example 202 in a racemic form, which was further separated by chiral HPLC to give:

Example 206 (100% ee); Retention time: 11.303 min. ¹H NMR (500 MHz, DMSO) δ 12.00 (s, 1H), 11.07 (s, 1H), 8.78 (s, 1H), 8.31 (t, J=7.8 Hz, 1H), 7.53 (d, J=8.6 Hz, 1H), 6.94 (d, J=7.5 Hz, 1H), 6.70 (s, 1H), 6.53 (d, J=14.0 Hz, 1H), 5.83 (s, 1H), 5.41 (s, 1H), 5.00 (s, 1H), 4.88-4.69 (m, 1H), 4.68-4.56 (m, 2H), 3.72 (d, J=11.5 Hz, 2H), 3.66-3.55 (m, 2H), 3.34 (s, 4H), 3.09-2.87 (m, 3H), 2.73-2.58 (m, 5H), 2.39-2.19 (m, 3H), 2.12-1.99 (m, 2H), 1.97-1.96 (m, 3H), 1.85-1.77 (m, 2H), 1.76-1.66 (d, 2H), 1.64-1.50 (d, 3H), 1.08-0.98 (m, 6H). LC-MS (ESI): m/z [M+H]⁺=897.77.

Chiral analytical method: Column: YMC Amylose-SA 4.6*250 mm 5 m Mobile phase: A for MtBE (0.1%2M NH3 MeOH)(%) and B for MeOH:DCM=50:50 (v/v); Gradient: Mobile Phase A: Mobile Phase B=40:60 (v/v); HPLC Equipment: Agilent 1260 Infinity II (ARD_LC-808); Column temperature: 25° C.

Chiral Prep-HPLC Condition: YMC Amylose-SA 4.6*250 mm 5 m Mobile phase: A for MtBE (0.1%2M NH3 MeOH)(%) and B for MeOH:DCM=50:50; Gradient: Mobile Phase A: Mobile Phase B=40:60 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 207: (1S,3R)-3-(3-((2-((3S,4S)-1′-(1-((S)-2,6-dioxopiperidin-3-yl)-7-fluoro-3-methyl-2-oxo-2,3-dihydro-1H-benzo[d]imidazol-5-yl)-3-fluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropyl carbamate

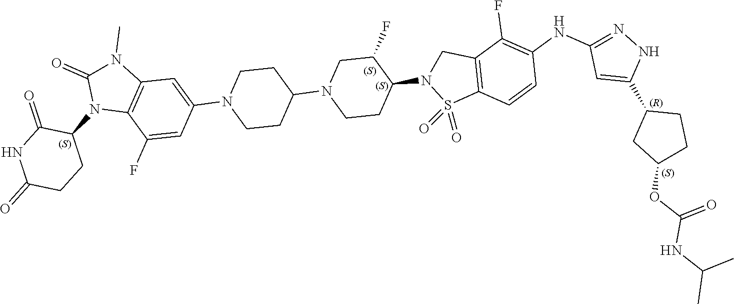

The titled compound was synthesized in the procedures similar to Example 202 in a racemic form, which was further separated by chiral HPLC to give:

Example 207 (100% ee); Retention time: 8.048 min. ¹H NMR (500 MHz, DMSO) δ 12.00 (s, 1H), 11.07 (s, 1H), 8.78 (s, 1H), 8.31 (t, J=7.8 Hz, 1H), 7.53 (d, J=8.6 Hz, 1H), 6.94 (d, J=7.5 Hz, 1H), 6.70 (s, 1H), 6.53 (d, J=14.0 Hz, 1H), 5.83 (s, 1H), 5.41 (s, 1H), 5.00 (s, 1H), 4.88-4.69 (m, 1H), 4.68-4.56 (m, 2H), 3.72 (d, J=11.5 Hz, 2H), 3.66-3.55 (m, 2H), 3.34 (s, 4H), 3.09-2.87 (m, 3H), 2.73-2.58 (m, 5H), 2.39-2.19 (m, 3H), 2.12-1.99 (m, 2H), 1.97-1.96 (m, 3H), 1.85-1.77 (m, 2H), 1.76-1.66 (d, 2H), 1.64-1.50 (d, 3H), 1.08-0.98 (m, 6H). LC-MS (ESI): m/z [M+H]⁺=897.89.

Chiral analytical method: Column: i-Cellulose-5 4.6*250 mm 5 m Mobile phase: A for MtBE (0.1%2M NH3 MeOH)(%) and B for MeOH:DCM=50:50 (v/v); Gradient: Mobile Phase A: Mobile Phase B=10:90 (v/v); HPLC Equipment: Agilent 1260 Infinity II (ARD_LC-808); Column temperature: 25° C.

Chiral Prep-HPLC Condition: i-Cellulose-5 4.6*250 mm 5 m Mobile phase: A for MtBE (0.1%2M NH3 MeOH)(%) and B for MeOH:DCM=50:50; Gradient: Mobile Phase A: Mobile Phase B=10:90 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 208: (1S,3R)-3-(3-((2-((3R,4S)-1′-(1-((S)-2,6-dioxopiperidin-3-yl)-7-fluoro-3-methyl-2-oxo-2,3-dihydro-1H-benzo[d]imidazol-5-yl)-3-fluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropyl carbamate

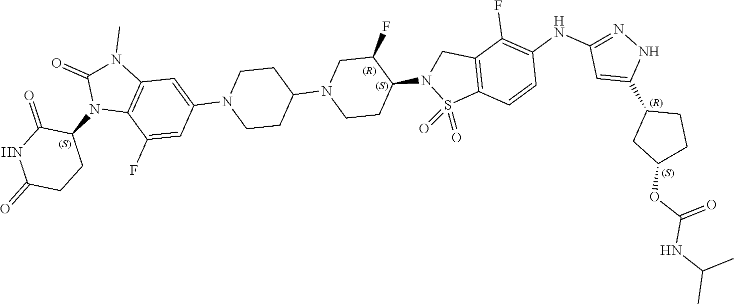

The titled compound was synthesized in the procedures similar to Example 202 in a racemic form, which was further separated by chiral HPLC to give:

Example 208 (100% ee); Retention time: 7.388 min. ¹H NMR (500 MHz, DMSO) δ 12.00 (s, 1H), 11.07 (s, 1H), 8.79 (s, 1H), 8.31 (t, J=7.9 Hz, 1H), 7.55 (d, J=8.7 Hz, 1H), 6.94 (d, J=7.4 Hz, 1H), 6.70 (s, 1H), 6.53 (d, J=13.7 Hz, 1H), 5.84 (s, 1H), 5.41 (s, 1H), 5.05-4.89 (m, 2H), 4.59 (dd, J=45.7, 14.0 Hz, 2H), 3.76-3.55 (m, 4H), 3.34 (s, 3H), 3.29 (s, 1H), 3.22-2.97 (m, 4H), 2.71-2.61 (m, 4H), 2.46-2.33 (m, 2H), 2.29-2.15 (m, 2H), 2.12-1.99 (m, 2H), 1.94-1.85 (m, 1H), 1.85-1.68 (m, 5H), 1.64-1.50 (m, 3H), 1.08-0.98 (m, 6H). LC-MS (ESI): m/z [M+H]⁺=897.69.

Chiral analytical method: Column: i-Cellulose-5 4.6*250 mm 5 m Mobile phase: A for MtBE (0.1%2M NH3 MeOH)(%) and B for MeOH:DCM=50:50 (v/v); Gradient: Mobile Phase A: Mobile Phase B=10:90 (v/v); HPLC Equipment: Agilent 1260 Infinity II (ARD_LC-808); Column temperature: 25° C.

Chiral Prep-HPLC Condition: i-Cellulose-5 4.6*250 mm 5 m Mobile phase: A for MtBE (0.1%2M NH3 MeOH)(%) and B for MeOH:DCM=50:50; Gradient: Mobile Phase A: Mobile Phase B=10:90 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 209: (1S,3R)-3-(3-((2-((3S,4R)-1′-(1-((S)-2,6-dioxopiperidin-3-yl)-7-fluoro-3-methyl-2-oxo-2,3-dihydro-1H-benzo[d]imidazol-5-yl)-3-fluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropyl carbamate

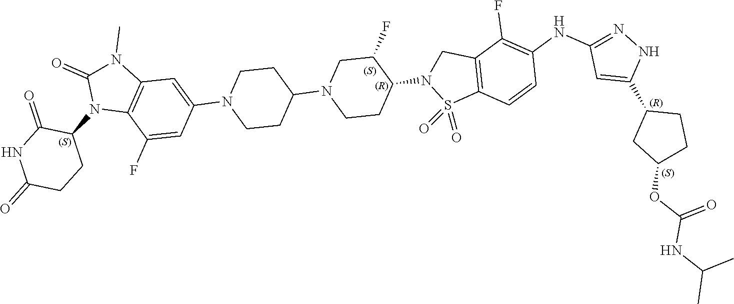

The titled compound was synthesized in the procedures similar to Example 202 in a racemic form, which was further separated by chiral HPLC to give:

Example 209 (100% ee); Retention time: 8.311 min. ¹H NMR (500 MHz, DMSO) δ 12.00 (s, 1H), 11.07 (s, 1H), 8.79 (s, 1H), 8.31 (t, J=7.9 Hz, 1H), 7.55 (d, J=8.7 Hz, 1H), 6.94 (d, J=7.4 Hz, 1H), 6.70 (s, 1H), 6.53 (d, J=13.7 Hz, 1H), 5.84 (s, 1H), 5.41 (s, 1H), 5.05-4.89 (m, 2H), 4.59 (dd, J=45.7, 14.0 Hz, 2H), 3.76-3.55 (m, 4H), 3.34 (s, 3H), 3.29 (s, 1H), 3.22-2.97 (m, 4H), 2.71-2.61 (m, 4H), 2.46-2.33 (m, 2H), 2.29-2.15 (m, 2H), 2.12-1.99 (m, 2H), 1.94-1.85 (m, 1H), 1.85-1.68 (m, 5H), 1.64-1.50 (m, 3H), 1.08-0.98 (m, 6H). LC-MS (ESI): m/z [M+H]⁺=897.60.

Chiral analytical method: Column: i-Cellulose-5 4.6*250 mm 5 m Mobile phase: A for MtBE (0.1%2M NH3 MeOH)(%) and B for MeOH:DCM=50:50 (v/v); Gradient: Mobile Phase A: Mobile Phase B=10:90 (v/v); HPLC Equipment: Agilent 1260 Infinity II (ARD_LC-808); Column temperature: 25° C.

Chiral Prep-HPLC Condition: i-Cellulose-5 4.6*250 mm 5 m Mobile phase: A for MtBE (0.1%2M NH3 MeOH)(%) and B for MeOH:DCM=50:50; Gradient: Mobile Phase A: Mobile Phase B=10:90 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 210: (1S,3R)-3-(3-((2-((3R,4S)-1′-(1-(2,6-dioxopiperidin-3-yl)-3,4-dimethyl-2-oxo-2,3-dihydro-1H-benzo[d]imidazol-5-yl)-3-methoxy-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

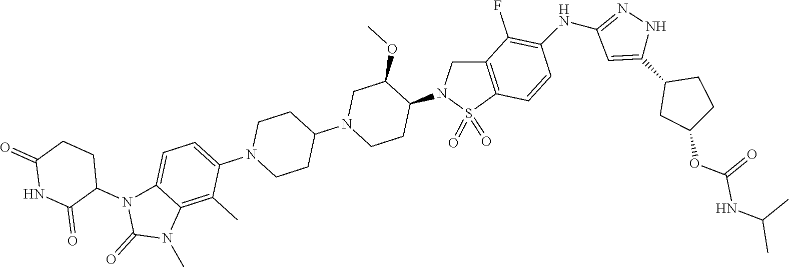 [SMILES: CO[C@@H]1CN(C2CCN(c3ccc4c(c3C)n(C)c(=O)n4C3CCC(=O)NC3=O)CC2)CC[C@@H]1N1Cc2c(ccc(Nc3cc([C@@H]4CC[C@H](OC(=O)NC(C)C)C4)[nH]n3)c2F)S1(=O)=O]

### Step 1: tert-butyl (3R,4S)-4-((4-bromo-2-(bromomethyl)-3-fluorophenyl)sulfonamido)-3-methoxypiperidine-1-carboxylate

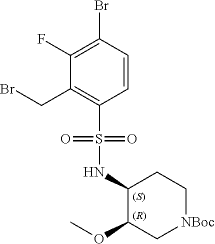

To a solution of To a solution of methyl 3-bromo-6-(chlorosulfonyl)-2-fluorobenzoate (1 g, 2.75 mmol) in ACN (20 mL), H2O (4 mL) was added tert-butyl (3R,4S)-4-amino-3-methoxypiperidine-1-carboxylate (632.5 mg, 2.75 mmol), and Na2CO3 (583 mg, 5.5 mmol). The reaction mixture was stirred for 3 hours at rt. The reaction was quenched by the addition of water (100 mL). The resulting mixture was extracted with EtOAc (2×100 mL), dried over anhydrous Na₂SO₄. The resulting mixture was concentrated under reduced pressure purified by silica gel column chromatography, eluting with PE/EA (3:1) to afford the product (1.38 g, 90%). LC-MS (ESI): m/z [M+H]⁺=559.2.

### Step 2: tert-butyl (3R,4S)-4-(5-bromo-4-fluoro-1,1-dioxidobenzo[d]isothiazol-2(3H)-yl)-3-methoxypiperidine-1-carboxylate

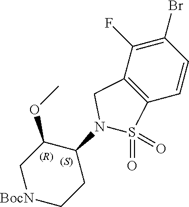

To a solution of To a solution of methyl 3-bromo-6-(chlorosulfonyl)-2-fluorobenzoate (1.38 g, 2.49 mmol) in ACN (20 mL), H2O (4 mL) was added Na2CO3 (523 mg, 4.93 mmol). The reaction mixture was stirred under nitrogen atmosphere at 80° C. for 3 h. The reaction was quenched by the addition of water (100 mL). The resulting mixture was extracted with EtOAc (2×100 mL), dried over anhydrous Na₂SO₄. The resulting mixture was concentrated under reduced pressure to afford the product (1.1 g, 90%). LC-MS (ESI): m/z [M+H]⁺=479.2.

### Step 3: tert-butyl (3R,4S)-4-(4-fluoro-5-((5-((1R,3S)-3-((isopropylcarbamoyl)oxy)cyclopentyl)-1H-pyrazol-3-yl)amino)-1,1-dioxidobenzo[d]isothiazol-2(3H)-yl)-3-methoxypiperidine-1-carboxylate

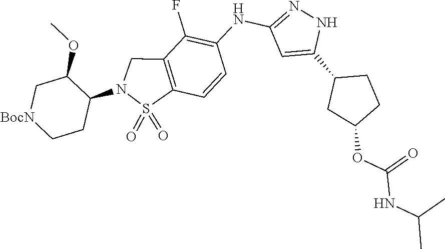 [SMILES: CO[C@@H]1CN(C(=O)OC(C)(C)C)CC[C@@H]1N1Cc2c(ccc(Nc3cc([C@@H]4CC[C@H](OC(=O)NC(C)C)C4)[nH]n3)c2F)S1(=O)=O]

To a mixture of tert-butyl (3R,4S)-4-(5-bromo-4-fluoro-1,1-dioxidobenzo[d]isothiazol-2(3H)-yl)-3-methoxypiperidine-1-carboxylate (300 mg, 0.63 mmol) and (1S,3R)-3-(3-amino-1H-pyrazol-5-yl)cyclopentyl isopropylcarbamate (166 mg, 0.63 mmol) in t-BuOH (10 mL) was added BrettPhos Pd G₃ (65 mg, 0.07 mmol) and K₂CO₃ (257 mg, 1.89 mmol). The reaction mixture was stirred under nitrogen atmosphere at 90° C. for 16 h. The mixture was filtered, and the filtrate was concentrated under reduced pressure. The residue was purified by silica gel column chromatography, eluting with DCM/MeOH (10:1) to afford the product (375 mg, 90% yield). LC-MS (ESI): m/z [M+H]⁺=665.3.

### Step 4: (1S,3R)-3-(3-((4-fluoro-2-((3R,4S)-3-methoxypiperidin-4-yl)-1,1-dioxido-2,3-dihydrobenzo[d]isothiazol-5-yl)amino)-1H-pyrazol-5-yl)cyclopentyl isopropylcarbamate

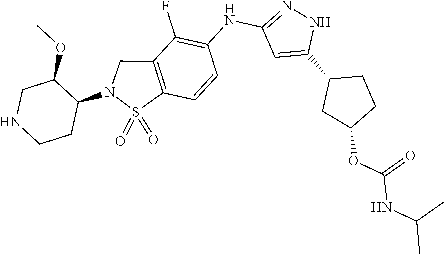 [SMILES: CO[C@@H]1CNCC[C@@H]1N1Cc2c(ccc(Nc3cc([C@@H]4CC[C@H](OC(=O)NC(C)C)C4)[nH]n3)c2F)S1(=O)=O]

tert-butyl (3R,4S)-4-(5-((5-((1S,3R)-3-((tert-butylcarbamoyl)oxy)cyclopentyl)-1H-pyrazol-3-yl)amino)-4-fluoro-1,1-dioxidobenzo[d]isothiazol-2(3H)-yl)-3-methoxypiperidine-1-carboxylate (300 mg, 0.45 mmol) was dissolved in 4M HCl (5 mL), the reaction mixture was stirred for 2 hours at room temperature. The resulting mixture was concentrated under reduce pressure. The resulting mixture was EA dilution adjusted PH to 7 with addition of TEA, a precipitated was washed with water, dried over in vacuum, to afford the product dried over in vacuum, to afford the product (252 mg, 100% yield). LC-MS (ESI): m/z [M+H]⁺=565.3.

### Step 5: (1S,3R)-3-(3-((2-((3R,4S)-1′-(1-(2,6-dioxopiperidin-3-yl)-3,4-dimethyl-2-oxo-2,3-dihydro-1H-benzo[d]imidazol-5-yl)-3-methoxy-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

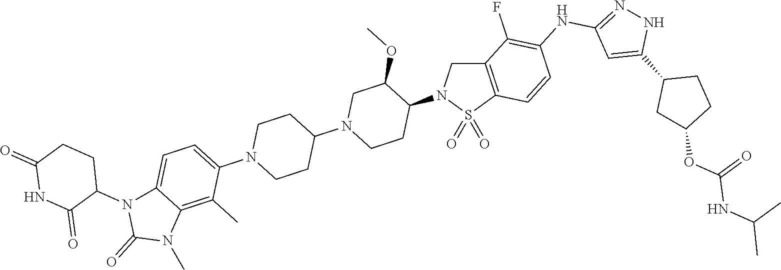 [SMILES: CO[C@@H]1CN(C2CCN(c3ccc4c(c3C)n(C)c(=O)n4C3CCC(=O)NC3=O)CC2)CC[C@@H]1N1Cc2c(ccc(Nc3cc([C@@H]4CC[C@H](OC(=O)NC(C)C)C4)[nH]n3)c2F)S1(=O)=O]

The titled compound was synthesized in the procedures similar to Example 43. ¹H NMR (500 MHz, DMSO) δ 12.00 (s, 1H), 11.06 (s, 1H), 8.76 (s, 1H), 8.29 (s, 1H), 7.53 (d, J=8.7 Hz, 1H), 6.94 (d, J=7.4 Hz, 1H), 6.86 (d, J=8.7 Hz, 1H), 6.78 (d, J=8.2 Hz, 1H), 5.84 (s, 1H), 5.31 (dd, J=12.7, 4.7 Hz, 1H), 4.99 (s, 1H), 4.67 (d, J=14.4 Hz, 1H), 4.56 (d, J=14.6 Hz, 1H), 3.69-3.64 (m, 1H), 3.61-3.52 (m, 5H), 3.29 (s, 3H), 3.12-3.04 (m, 1H), 3.00-2.95 (m, 3H), 2.91-2.84 (m, 1H), 2.72-2.66 (m, 2H), 2.64-2.57 (m, 8H), 2.42-2.38 (m, 2H), 2.28-2.18 (m, 2H), 2.08-1.78 (m, 6H), 1.75-1.55 (m, 6H), 1.03 (d, J=6.1 Hz, 6H). LC-MS (ESI): m/z [M+H]⁺=905.4;

### Example 211: (1S,3R)-3-(3-((2-((3S,4R)-1′-(1-(2,6-dioxopiperidin-3-yl)-3,4-dimethyl-2-oxo-2,3-dihydro-1H-benzo[d]imidazol-5-yl)-3-methoxy-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

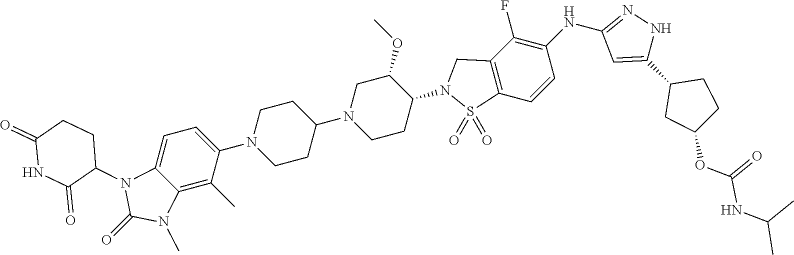 [SMILES: CO[C@H]1CN(C2CCN(c3ccc4c(c3C)n(C)c(=O)n4C3CCC(=O)NC3=O)CC2)CC[C@H]1N1Cc2c(ccc(Nc3cc([C@@H]4CC[C@H](OC(=O)NC(C)C)C4)[nH]n3)c2F)S1(=O)=O]

The titled compound was synthesized in the procedures similar to Example 210. ¹H NMR (500 MHz, DMSO) δ 12.00 (s, 1H), 11.06 (s, 1H), 8.76 (s, 1H), 8.29 (s, 1H), 7.53 (d, J=8.7 Hz, 1H), 6.94 (d, J=7.4 Hz, 1H), 6.86 (d, J=8.7 Hz, 1H), 6.78 (d, J=8.2 Hz, 1H), 5.84 (s, 1H), 5.31 (dd, J=12.7, 4.7 Hz, 1H), 4.99 (s, 1H), 4.67 (d, J=14.4 Hz, 1H), 4.56 (d, J=14.6 Hz, 1H), 3.69-3.64 (m, 1H), 3.61-3.49 (m, 5H), 3.29 (s, 3H), 3.12-3.04 (m, 1H), 3.00-2.95 (m, 3H), 2.91-2.84 (m, 1H), 2.72-2.66 (m, 2H), 2.64-2.57 (m, 8H), 2.42-2.38 (m, 2H), 2.28-2.18 (m, 2H), 2.08-1.78 (m, 6H), 1.75-1.55 (m, 6H), 1.03 (d, J=6.1 Hz, 6H). LC-MS (ESI): m/z [M+H]⁺=905.4;

### Example 212 and 213: (1S,3R)-3-(3-((2-((3R,4S)-1-((1-(1-((S)-2,6-dioxopiperidin-3-yl)-7-fluoro-3-methyl-2-oxo-2,3-dihydro-1H-benzo[d]imidazol-5-yl)piperidin-4-yl)methyl)-3-methoxypiperidin-4-yl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-((3R,4S)-1-((1-(1-((R)-2,6-dioxopiperidin-3-yl)-7-fluoro-3-methyl-2-oxo-2,3-dihydro-1H-benzo[d]imidazol-5-yl)piperidin-4-yl)methyl)-3-methoxypiperidin-4-yl)-4-fluoro-1,1-dioxido-2,3-dihydrobenzo[d]isothiazol-5-yl)amino)-1H-pyrazol-5-yl)cyclopentyl isopropylcarbamate

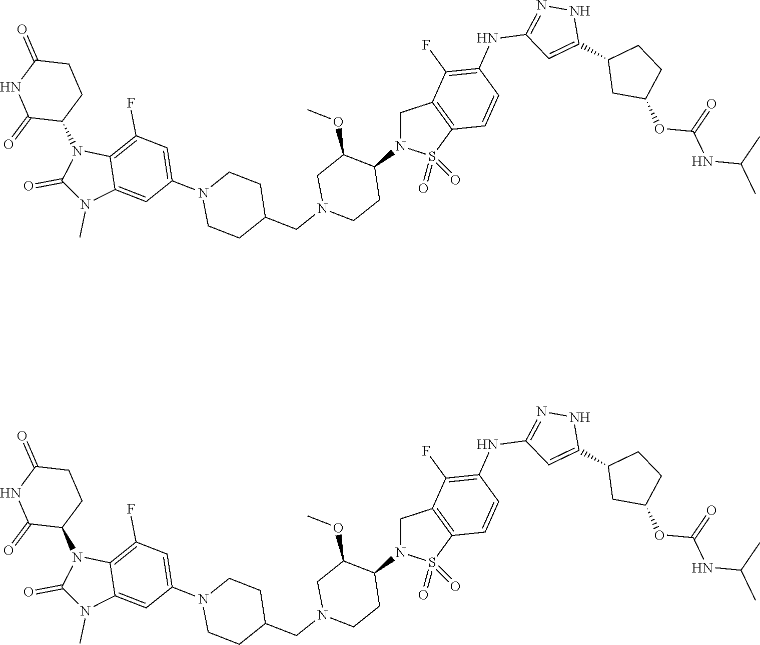 [SMILES: CO[C@@H]1CN(CC2CCN(c3cc(F)c4c(c3)n(C)c(=O)n4[C@H]3CCC(=O)NC3=O)CC2)CC[C@@H]1N1Cc2c(ccc(Nc3cc([C@@H]4CC[C@H](OC(=O)NC(C)C)C4)[nH]n3)c2F)S1(=O)=O ; CO[C@@H]1CN(CC2CCN(c3cc(F)c4c(c3)n(C)c(=O)n4[C@@H]3CCC(=O)NC3=O)CC2)CC[C@@H]1N1Cc2c(ccc(Nc3cc([C@@H]4CC[C@H](OC(=O)NC(C)C)C4)[nH]n3)c2F)S1(=O)=O]

The titled compound was synthesized in the procedures similar to Example 210 in a racemate form, which was further separated by chiral HPLC to give:

Enantiomer 1 (Example 212, 100% ee); Retention time: 6.394 min. ¹H NMR (500 MHz, DMSO) δ 11.99 (s, 1H), 11.07 (s, 1H), 8.77 (s, 1H), 8.30 (t, J=7.9 Hz, 1H), 7.53 (d, J=8.7 Hz, 1H), 6.94 (d, J=7.3 Hz, 1H), 6.68 (s, 1H), 6.52 (d, J=15.0 Hz, 1H), 5.84 (s, 1H), 5.41 (s, 1H), 5.06-4.96 (m, 1H), 4.67 (d, J=14.4 Hz, 1H), 4.56 (d, J=14.5 Hz, 1H), 3.72-3.65-3.53 (m, 3H), 3.61-3.54 (m, 2H), 3.34 (s, 2H), 3.30 (s, 3H), 3.21 (d, J=12.0 Hz, 1H), 3.13-2.86 (m, 3H), 2.71-2.62 (m, 4H), 2.46-2.43 (m, 1H), 2.31-2.15 (m, 4H), 2.14-1.97 (m, 4H), 1.94-1.57 (m, 8H), 1.28-1.26 (m, 2H), 1.03 (d, J=6.3 Hz, 6H). LC-MS (ESI): m/z [M+H]+=923.4;

Enantiomer 2 (Example 213, 100% ee); Retention time: 11.499 min. ¹H NMR (500 MHz, DMSO) δ 11.99 (s, 1H), 11.07 (s, 1H), 8.77 (s, 1H), 8.30 (t, J=7.9 Hz, 1H), 7.53 (d, J=8.7 Hz, 1H), 6.94 (d, J=7.3 Hz, 1H), 6.68 (s, 1H), 6.52 (d, J=15.0 Hz, 1H), 5.84 (s, 1H), 5.41 (s, 1H), 5.06-4.96 (m, 1H), 4.67 (d, J=14.4 Hz, 1H), 4.56 (d, J=14.5 Hz, 1H), 3.72-3.65-3.53 (m, 3H), 3.61-3.54 (m, 2H), 3.34 (s, 2H), 3.30 (s, 3H), 3.21 (d, J=12.0 Hz, 1H), 3.13-2.86 (m, 3H), 2.71-2.62 (m, 4H), 2.46-2.43 (m, 1H), 2.31-2.15 (m, 4H), 2.14-1.97 (m, 4H), 1.94-1.57 (m, 8H), 1.28-1.26 (m, 2H), 1.03 (d, J=6.3 Hz, 6H). LC-MS (ESI): m/z [M+H]+=923.4;

Chiral analytical method: Column: ICHIRALPAK IC-5 4.6*150 mm 5 m; Mobile phase: A for MtBE (0.1%2M NH3 MeOH) (%) and B for MeOH:DCM=50:50(%); Gradient: Mobile Phase A: Mobile Phase B=30:70 (v/v); HPLC Equipment: HPLC-Agilent, Back pressure: 100 bar; Column temperature: 35° C.

Chiral Prep-HPLC Condition: CHIRALPAK IC-5 250*20.0 mml, 5 μm; Mobile phase: A for MtBE (0.1%2M NH3 MeOH) (%) and B for MeOH:DCM=50:50; Gradient: Mobile Phase A: Mobile Phase B=30:70 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 214 and 215: (1S,3R)-3-(3-((2-((3R,4S)-1′-(1-((S)-2,6-dioxopiperidin-3-yl)-7-fluoro-3-methyl-2-oxo-2,3-dihydro-1H-benzo[d]imidazol-5-yl)-3-methoxy-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-((3R,4S)-1′-(1-((R)-2,6-dioxopiperidin-3-yl)-7-fluoro-3-methyl-2-oxo-2,3-dihydro-1H-benzo[d]imidazol-5-yl)-3-methoxy-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

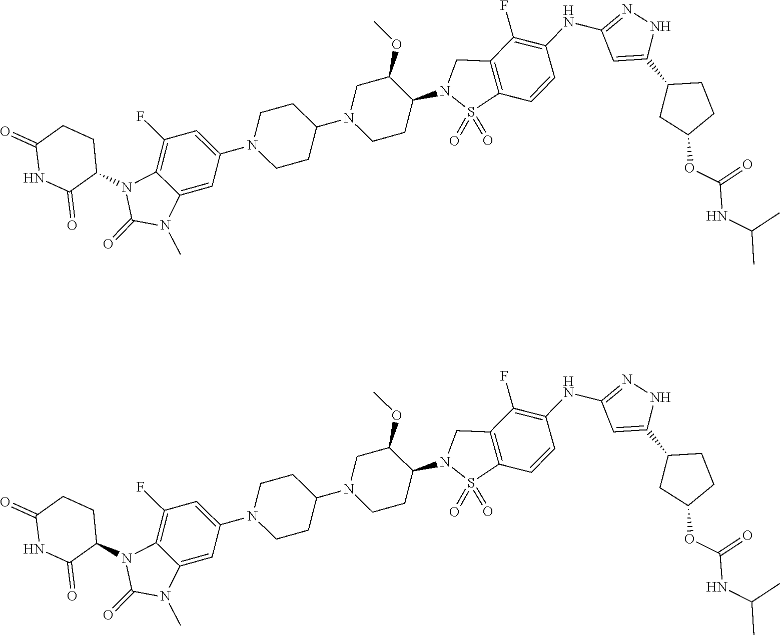 [SMILES: CO[C@@H]1CN(C2CCN(c3cc(F)c4c(c3)n(C)c(=O)n4[C@H]3CCC(=O)NC3=O)CC2)CC[C@@H]1N1Cc2c(ccc(Nc3cc([C@@H]4CC[C@H](OC(=O)NC(C)C)C4)[nH]n3)c2F)S1(=O)=O ; CO[C@@H]1CN(C2CCN(c3cc(F)c4c(c3)n(C)c(=O)n4[C@@H]3CCC(=O)NC3=O)CC2)CC[C@@H]1N1Cc2c(ccc(Nc3cc([C@@H]4CC[C@H](OC(=O)NC(C)C)C4)[nH]n3)c2F)S1(=O)=O]

The titled compound was synthesized in the procedures similar to Example 210 in a racemate form, which was further separated by chiral HPLC to give:

Enantiomer 1 (Example 214, 100% ee); Retention time: 9.159 min. ¹H NMR (500 MHz, DMSO) δ 11.99 (s, 1H), 11.07 (s, 1H), 8.77 (s, 1H), 8.30 (t, J=7.9 Hz, 1H), 7.53 (d, J=8.7 Hz, 1H), 6.94 (d, J=7.3 Hz, 1H), 6.68 (s, 1H), 6.52 (d, J=15.0 Hz, 1H), 5.84 (s, 1H), 5.41 (s, 1H), 5.06-4.96 (m, 1H), 4.67 (d, J=14.4 Hz, 1H), 4.56 (d, J=14.5 Hz, 1H), 3.72-3.65-3.53 (m, 3H), 3.61-3.54 (m, 2H), 3.34 (s, 2H), 3.30 (s, 3H), 3.21 (d, J=12.0 Hz, 1H), 3.13-2.86 (m, 3H), 2.71-2.62 (m, 4H), 2.46-2.43 (m, 1H), 2.31-2.15 (m, 4H), 2.14-1.97 (m, 4H), 1.94-1.57 (m, 8H), 1.28-1.26 (m, 2H), 1.03 (d, J=6.3 Hz, 6H). LC-MS (ESI): m/z [M+H]+=909.4;

Enantiomer 2 (Example 215, 100% ee); Retention time: 12.477 min. ¹H NMR (500 MHz, DMSO) δ 11.99 (s, 1H), 11.07 (s, 1H), 8.77 (s, 1H), 8.30 (t, J=7.9 Hz, 1H), 7.53 (d, J=8.7 Hz, 1H), 6.94 (d, J=7.3 Hz, 1H), 6.68 (s, 1H), 6.52 (d, J=15.0 Hz, 1H), 5.84 (s, 1H), 5.41 (s, 1H), 5.06-4.96 (m, 1H), 4.67 (d, J=14.4 Hz, 1H), 4.56 (d, J=14.5 Hz, 1H), 3.72-3.65-3.53 (m, 3H), 3.61-3.54 (m, 2H), 3.34 (s, 2H), 3.30 (s, 3H), 3.21 (d, J=12.0 Hz, 1H), 3.13-2.86 (m, 3H), 2.71-2.62 (m, 4H), 2.46-2.43 (m, 1H), 2.31-2.15 (m, 4H), 2.14-1.97 (m, 4H), 1.94-1.57 (m, 8H), 1.28-1.26 (m, 2H), 1.03 (d, J=6.3 Hz, 6H). LC-MS (ESI): m/z [M+H]+=909.4;

Chiral analytical method: Column: ICHIRALPAK IC-5 4.6*150 mm 5 m; Mobile phase: A for MtBE (0.1%2M NH3 MeOH) (%) and B for MeOH:DCM=50:50(%); Gradient: Mobile Phase A: Mobile Phase B=30:70 (v/v); HPLC Equipment: HPLC-Agilent, Back pressure: 100 bar; Column temperature: 35° C.

Chiral Prep-HPLC Condition: CHIRALPAK IC-5 250*20.0 mml, 5 μm; Mobile phase: A for MtBE (0.1%2M NH3 MeOH) (%) and B for MeOH:DCM=50:50; Gradient: Mobile Phase A: Mobile Phase B=30:70 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 216 and 217: (1S,3R)-3-(3-((2-((3S,4R)-1-((1-(1-((S)-2,6-dioxopiperidin-3-yl)-7-fluoro-3-methyl-2-oxo-2,3-dihydro-1H-benzo[d]imidazol-5-yl)piperidin-4-yl)methyl)-3-methoxypiperidin-4-yl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-((3S,4R)-1-((1-(1-((R)-2,6-dioxopiperidin-3-yl)-7-fluoro-3-methyl-2-oxo-2,3-dihydro-1H-benzo[d]imidazol-5-yl)piperidin-4-yl)methyl)-3-methoxypiperidin-4-yl)-4-fluoro-1,1-dioxido-2,3-dihydrobenzo[d]isothiazol-5-yl)amino)-1H-pyrazol-5-yl)cyclopentyl isopropylcarbamate

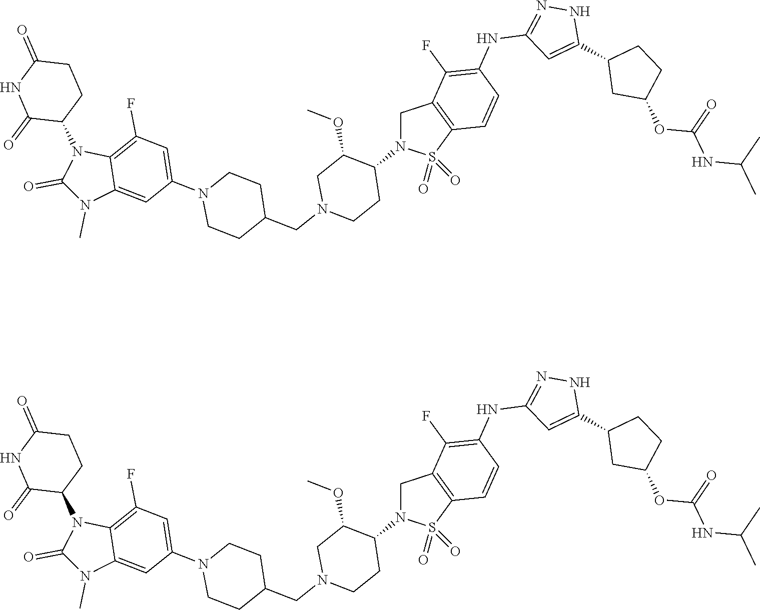 [SMILES: CO[C@H]1CN(CC2CCN(c3cc(F)c4c(c3)n(C)c(=O)n4[C@H]3CCC(=O)NC3=O)CC2)CC[C@H]1N1Cc2c(ccc(Nc3cc([C@@H]4CC[C@H](OC(=O)NC(C)C)C4)[nH]n3)c2F)S1(=O)=O ; CO[C@H]1CN(CC2CCN(c3cc(F)c4c(c3)n(C)c(=O)n4[C@@H]3CCC(=O)NC3=O)CC2)CC[C@H]1N1Cc2c(ccc(Nc3cc([C@@H]4CC[C@H](OC(=O)NC(C)C)C4)[nH]n3)c2F)S1(=O)=O]

The titled compound was synthesized in the procedures similar to Example 210 in a racemate form, which was further separated by chiral HPLC to give:

Enantiomer 1 (Example 216, 100% ee); Retention time: 6.371 min. ¹H NMR (500 MHz, DMSO) δ 11.99 (s, 1H), 11.07 (s, 1H), 8.77 (s, 1H), 8.30 (t, J=7.9 Hz, 1H), 7.53 (d, J=8.7 Hz, 1H), 6.94 (d, J=7.3 Hz, 1H), 6.68 (s, 1H), 6.52 (d, J=15.0 Hz, 1H), 5.84 (s, 1H), 5.41 (s, 1H), 5.06-4.96 (m, 1H), 4.67 (d, J=14.4 Hz, 1H), 4.56 (d, J=14.5 Hz, 1H), 3.72-3.65-3.53 (m, 3H), 3.61-3.54 (m, 2H), 3.34 (s, 2H), 3.30 (s, 3H), 3.21 (d, J=12.0 Hz, 1H), 3.13-2.86 (m, 3H), 2.71-2.62 (m, 4H), 2.46-2.43 (m, 1H), 2.31-2.15 (m, 4H), 2.14-1.97 (m, 4H), 1.94-1.57 (m, 8H), 1.28-1.26 (m, 2H), 1.03 (d, J=6.3 Hz, 6H). LC-MS (ESI): m/z [M+H]+=923.4;

Enantiomer 1 (Example 217, 100% ee); Retention time: 11.702 min. ¹¹H NMR (500 MHz, DMSO) δ 11.99 (s, 1H), 11.07 (s, 1H), 8.77 (s, 1H), 8.30 (t, J=7.9 Hz, 1H), 7.53 (d, J=8.7 Hz, 1H), 6.94 (d, J=7.3 Hz, 1H), 6.68 (s, 1H), 6.52 (d, J=15.0 Hz, 1H), 5.84 (s, 1H), 5.41 (s, 1H), 5.06-4.96 (m, 1H), 4.67 (d, J=14.4 Hz, 1H), 4.56 (d, J=14.5 Hz, 1H), 3.72-3.65-3.53 (m, 3H), 3.61-3.54 (m, 2H), 3.34 (s, 2H), 3.30 (s, 3H), 3.21 (d, J=12.0 Hz, 1H), 3.13-2.86 (m, 3H), 2.71-2.62 (m, 4H), 2.46-2.43 (m, 1H), 2.31-2.15 (m, 4H), 2.14-1.97 (m, 4H), 1.94-1.57 (m, 8H), 1.28-1.26 (m, 2H), 1.03 (d, J=6.3 Hz, 6H). LC-MS (ESI): m/z [M+H]+=923.4;

Chiral analytical method: Column: ICHIRALPAK IC-5 4.6*150 mm 5 m; Mobile phase: A for MtBE (0.1%2M NH3 MeOH) (%) and B for MeOH:DCM=50:50(%); Gradient: Mobile Phase A: Mobile Phase B=30:70 (v/v); HPLC Equipment: HPLC-Agilent, Back pressure: 100 bar; Column temperature: 35° C.

Chiral Prep-HPLC Condition: CHIRALPAK IC-5 250*20.0 mml, 5 μm; Mobile phase: A for MtBE (0.1%2M NH3 MeOH) (%) and B for MeOH:DCM=50:50; Gradient: Mobile Phase A: Mobile Phase B=30:70 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 218 and 219: (1S,3R)-3-(3-((2-((3S,4R)-1′-(1-((S)-2,6-dioxopiperidin-3-yl)-7-fluoro-3-methyl-2-oxo-2,3-dihydro-1H-benzo[d]imidazol-5-yl)-3-methoxy-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-((3S,4R)-1′-(1-((R)-2,6-dioxopiperidin-3-yl)-7-fluoro-3-methyl-2-oxo-2,3-dihydro-1H-benzo[d]imidazol-5-yl)-3-methoxy-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

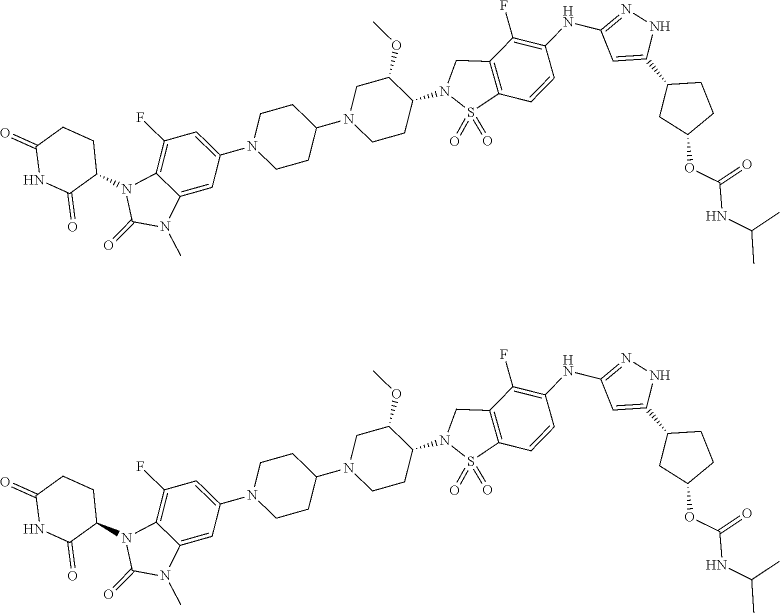 [SMILES: CO[C@H]1CN(C2CCN(c3cc(F)c4c(c3)n(C)c(=O)n4[C@H]3CCC(=O)NC3=O)CC2)CC[C@H]1N1Cc2c(ccc(Nc3cc([C@@H]4CC[C@H](OC(=O)NC(C)C)C4)[nH]n3)c2F)S1(=O)=O ; CO[C@H]1CN(C2CCN(c3cc(F)c4c(c3)n(C)c(=O)n4[C@@H]3CCC(=O)NC3=O)CC2)CC[C@H]1N1Cc2c(ccc(Nc3cc([C@@H]4CC[C@H](OC(=O)NC(C)C)C4)[nH]n3)c2F)S1(=O)=O]

The titled compound was synthesized in the procedures similar to Example 210 in a racemate form, which was further separated by chiral HPLC to give:

Enantiomer 1 (Example 218, 100% ee); Retention time: 9.574 min. ¹H NMR (500 MHz, DMSO) δ 11.99 (s, 1H), 11.07 (s, 1H), 8.77 (s, 1H), 8.30 (t, J=7.9 Hz, 1H), 7.53 (d, J=8.7 Hz, 1H), 6.94 (d, J=7.3 Hz, 1H), 6.68 (s, 1H), 6.52 (d, J=15.0 Hz, 1H), 5.84 (s, 1H), 5.41 (s, 1H), 5.06-4.96 (m, 1H), 4.67 (d, J=14.4 Hz, 1H), 4.56 (d, J=14.5 Hz, 1H), 3.72-3.65-3.53 (m, 3H), 3.61-3.54 (m, 2H), 3.34 (s, 2H), 3.30 (s, 3H), 3.21 (d, J=12.0 Hz, 1H), 3.13-2.86 (m, 3H), 2.71-2.62 (m, 6H), 2.46-2.43 (m, 1H), 2.31-2.15 (m, 4H), 2.14-1.97 (m, 4H), 1.94-1.57 (m, 8H), 1.28-1.26 (m, 2H), 1.03 (d, J=6.3 Hz, 6H). LC-MS (ESI): m/z [M+H]+=909.4;

Enantiomer 1 (Example 219, 100% ee); Retention time: 12.066 min. ¹H NMR (500 MHz, DMSO) δ 11.99 (s, 1H), 11.07 (s, 1H), 8.77 (s, 1H), 8.30 (t, J=7.9 Hz, 1H), 7.53 (d, J=8.7 Hz, 1H), 6.94 (d, J=7.3 Hz, 1H), 6.68 (s, 1H), 6.52 (d, J=15.0 Hz, 1H), 5.84 (s, 1H), 5.41 (s, 1H), 5.06-4.96 (m, 1H), 4.67 (d, J=14.4 Hz, 1H), 4.56 (d, J=14.5 Hz, 1H), 3.72-3.65-3.53 (m, 3H), 3.61-3.54 (m, 2H), 3.34 (s, 2H), 3.30 (s, 3H), 3.21 (d, J=12.0 Hz, 1H), 3.13-2.86 (m, 3H), 2.71-2.62 (m, 6H), 2.46-2.43 (m, 1H), 2.31-2.15 (m, 4H), 2.14-1.97 (m, 4H), 1.94-1.57 (m, 8H), 1.28-1.26 (m, 2H), 1.03 (d, J=6.3 Hz, 6H). LC-MS (ESI): m/z [M+H]+=909.4;

Chiral analytical method: Column: ICHIRALPAK IC-5 4.6*150 mm 5 m; Mobile phase: A for MtBE (0.1%2M NH3 MeOH) (%) and B for MeOH:DCM=50:50(%); Gradient: Mobile Phase A: Mobile Phase B=30:70 (v/v); HPLC Equipment: HPLC-Agilent, Back pressure: 100 bar; Column temperature: 35° C.

Chiral Prep-HPLC Condition: CHIRALPAK IC-5 250*20.0 mml, 5 μm; Mobile phase: A for MtBE (0.1%2M NH3 MeOH) (%) and B for MeOH:DCM=50:50; Gradient: Mobile Phase A: Mobile Phase B=30:70 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 220: (1S,3R)-3-(3-((2-((S)-1-((1-(1-((R)-2,6-dioxopiperidin-3-yl)-4-fluoro-3-methyl-2-oxo-2,3-dihydro-1H-benzo[d]imidazol-5-yl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropyl carbamate

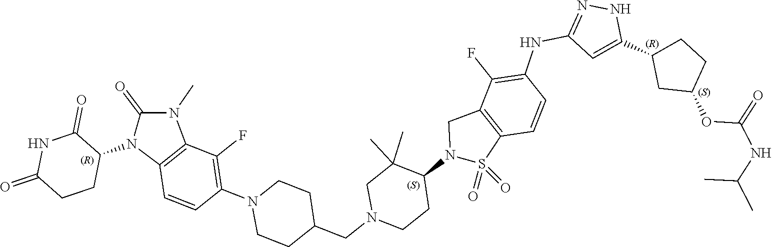

The titled compound was synthesized in the procedures similar to Example 37 in a racemate form, which was further separated by chiral HPLC to give:

Example 220 (100% ee); Retention time: 5.44 min. ¹H NMR (500 MHz, DMSO) δ 11.99 (s, 1H), 11.10 (s, 1H), 8.77 (s, 1H), 8.31 (t, J=7.9 Hz, 1H), 7.54 (d, J=8.6 Hz, 1H), 6.94 (d, J=7.4 Hz, 1H), 6.85 (d, J=8.5 Hz, 1H), 6.74 (t, J=8.2 Hz, 1H), 5.83 (s, 1H), 5.33 (dd, J=12.6, 5.4 Hz, 1H), 5.00 (s, 1H), 4.53 (m, J=14.9 Hz, 2H), 3.64-3.53 (m, 1H), 3.47 (s, 3H), 3.28-3.21 (m, 3H), 3.10-3.02 (m, 1H), 2.98-2.83 (m, 2H), 2.73-2.61 (m, 4H), 2.24-2.10 (m, 3H), 2.06-1.67 (m, 10H), 1.60 (s, 2H), 1.51 (d, J=10.4 Hz, 1H), 1.27 (m, 3H), 1.13 (s, 3H), 1.08-1.00 (m, 6H), 0.94 (s, 3H). LC-MS (ESI): m/z [M+H]⁺=921.63.

Chiral analytical method: Column: YMC Amylose-SA 4.6*250 mm 5 m Mobile phase: A for MtBE (0.1%2M NH3 MeOH) (%) and B for MeOH:DCM=50:50 (v/v); Gradient: Mobile Phase A: Mobile Phase B=30:70 (v/v); HPLC Equipment: Agilent 1260 Infinity II (ARD_LC-808); Column temperature: 25° C.

Chiral Prep-HPLC Condition: YMC Amylose-SA 4.6*250 mm 5 m Mobile phase: A for MtBE (0.1%2M NH3 MeOH)(%) and B for MeOH:DCM=50:50; Gradient: Mobile Phase A: Mobile Phase B=30:70 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 221: (1S,3R)-3-(3-((2-((4S)-1-((1-(1-(2,6-dioxopiperidin-3-yl)-3-methyl-2-oxo-2,3-dihydro-1H-imidazo[4,5-b]pyridin-5-yl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropylcarbamate

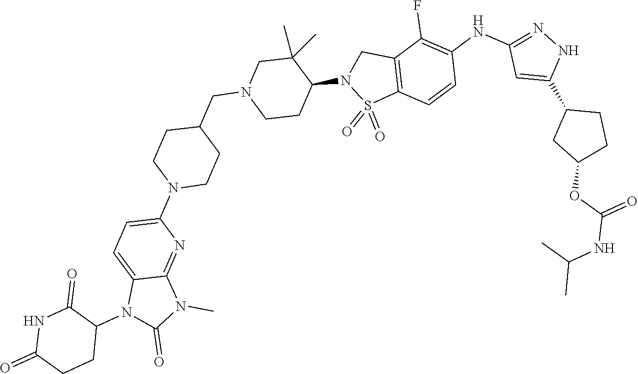 [SMILES: CC(C)NC(=O)O[C@H]1CC[C@@H](c2cc(Nc3ccc4c(c3F)CN([C@H]3CCN(CC5CCN(c6ccc7c(n6)n(C)c(=O)n7C6CCC(=O)NC6=O)CC5)CC3(C)C)S4(=O)=O)n[nH]2)C1]

The titled compound was synthesized in the procedures similar to Example 53 in a racemate form, which was further separated by chiral HPLC to give Example 221 (100% ee); Retention time: 4.89 min. ¹H NMR (500 MHz, DMSO) δ 11.98 (s, 1H), 11.07 (s, 1H), 8.76 (s, 1H), 8.30 (s, 1H), 7.54 (d, J=8.7 Hz, 1H), 7.29 (d, J=8.5 Hz, 1H), 6.95 (s, 1H), 6.44 (d, J=8.4 Hz, 1H), 5.83 (s, 1H), 5.30 (d, J=13.4 Hz, 1H), 5.00 (s, 1H), 4.54 (s, 2H), 4.21-4.18 (m, 2H), 3.58 (s, 1H), 3.29-3.28 (m, 5H), 3.23-3.21 (m, 1H), 3.06 (s, 1H), 3.00-2.84 (m, 3H), 2.75-2.73 (m, 3H), 2.67-2.64 (m, 6H), 2.14-2.09 (m, 6H), 2.05-1.86 (m, 3H), 1.85-1.65 (m, 3H), 1.24-1.09 (m, 6H), 1.03-1.02 (m, 6H), 0.93 (s, 3H). LC-MS (ESI): m/z [M+H]+=904.57.

Chiral analytical method: Column: YMC Amylose-SA 4.6*250 mm 5 m; Mobile phase: A for MtBE (0.1%2M NH3 MeOH)(%) and B for DCM:MeOH=50:50(%); Gradient: Mobile Phase A: Mobile Phase B=40:60 (v/v); HPLC Equipment: HPLC-Agilent, Back pressure: 100 bar; Column temperature: 35° C.

Chiral Prep-HPLC Condition: Column: YMC Amylose-SA 4.6*250 mm 5 m; Mobile phase: A MtBE (0.1%2M NH3 MeOH)(%) and B for DCM:MeOH=50:50(%); Gradient: Mobile Phase A: Mobile Phase B=40:60 (v/v); Flow Rate: 20 mL/min, Wave Length: UV 200 nm and 270 nm, Prep-HPLC Equipment: Prep-HPLC-Gilson, Back pressure: 100 bar; Column temperature: 25° C.

### Example 222: (1S,3R)-3-(3-((2-((4S)-1-((1-(1-(2,6-dioxopiperidin-3-yl)-3,4-dimethyl-2-oxo-2,3-dihydro-1H-benzo[d]imidazol-5-yl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropylcarbamate

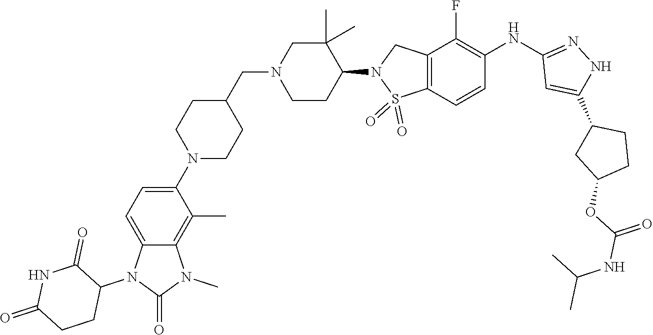 [SMILES: Cc1c(N2CCC(CN3CC[C@H](N4Cc5c(ccc(Nc6cc([C@@H]7CC[C@H](OC(=O)NC(C)C)C7)[nH]n6)c5F)S4(=O)=O)C(C)(C)C3)CC2)ccc2c1n(C)c(=O)n2C1CCC(=O)NC1=O]

The titled compound was synthesized in the procedures similar to Example 54. ¹H NMR (500 MHz, DMSO) δ 11.99 (s, 1H), 11.06 (s, 1H), 8.76 (s, 1H), 8.29 (s, 1H), 7.54 (d, J=8.6 Hz, 1H), 6.94 (d, J=7.5 Hz, 1H), 6.86 (d, J=8.3 Hz, 1H), 6.78 (d, J=8.5 Hz, 1H), 5.83 (s, 1H), 5.31 (dd, J=12.8, 5.2 Hz, 1H), 5.00 (s, 1H), 4.62-4.42 (m, 2H), 3.58 (s, 4H), 3.25-3.21 (m, 1H), 3.13-3.01 (m, 1H), 3.01-2.83 (m, 4H), 2.72-2.60 (m, 4H), 2.58-2.33 (m, 5H), 2.23-2.12 (m, 3H), 2.06-1.94 (m, 3H), 1.94-1.88 (m, 1H), 1.85-1.77 (m, 3H), 1.73-1.68 (m, 2H), 1.59 (s, 2H), 1.52-1.50 (m, 1H), 1.31-1.29 (dm, 2H), 1.13 (s, 3H), 1.07-0.99 (m, 6H), 0.94 (s, 3H). LC-MS (ESI): m/z [M+H]⁺=917.49.

### Example 223: (1S,3R)-3-(3-((2-((4R)-1′-(1-(2,6-dioxopiperidin-3-yl)-3,4-dimethyl-2-oxo-2,3-dihydro-1H-benzo[d]imidazol-5-yl)-3,3-dimethyl-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

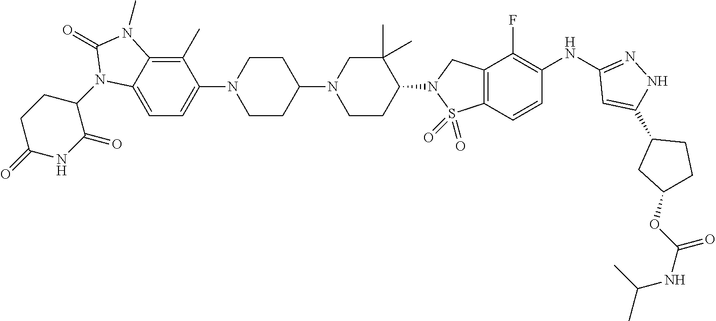 [SMILES: Cc1c(N2CCC(N3CC[C@@H](N4Cc5c(ccc(Nc6cc([C@@H]7CC[C@H](OC(=O)NC(C)C)C7)[nH]n6)c5F)S4(=O)=O)C(C)(C)C3)CC2)ccc2c1n(C)c(=O)n2C1CCC(=O)NC1=O]

The titled compound was synthesized in the procedures similar to Example 43. ¹H NMR (500 MHz, DMSO) δ 11.99 (s, 1H), 11.06 (s, 1H), 8.77 (s, 1H), 8.30 (s, 1H), 7.54 (d, J=8.6 Hz, 1H), 6.94 (d, J=7.3 Hz, 1H), 6.86 (d, J=8.5 Hz, 1H), 6.78 (d, J=8.4 Hz, 1H), 5.84 (s, 1H), 5.31 (dd, J=12.7, 5.2 Hz, 1H), 5.00 (s, 1H), 4.54 (s, 2H), 3.58 (s, 4H), 3.23-3.20 (m, 1H), 3.12-3.02 (m, 2H), 2.98-2.96 (m, 2H), 2.91-2.85 (m, 1H), 2.76-2.65 (m, 2H), 2.65-2.57 (m, 4H), 2.54 (s, 4H), 2.42-2.25 (m, 2H), 2.19-1.87 (m, 4H), 1.85-1.48 (m, 8H), 1.11 (s, 3H), 1.03-1.02 (m, 6H), 0.96 (s, 3H). LC-MS (ESI): m/z [M+H]⁺=903.6.

### Example 224: (1S,3R)-3-(3-((2-((4S)-1-(1-((1-(1-(2,6-dioxopiperidin-3-yl)-4-fluoro-3-methyl-2-oxo-2,3-dihydro-1H-benzo[d]imidazol-5-yl)piperidin-4-yl)methyl)azetidin-3-yl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropylcarbamate

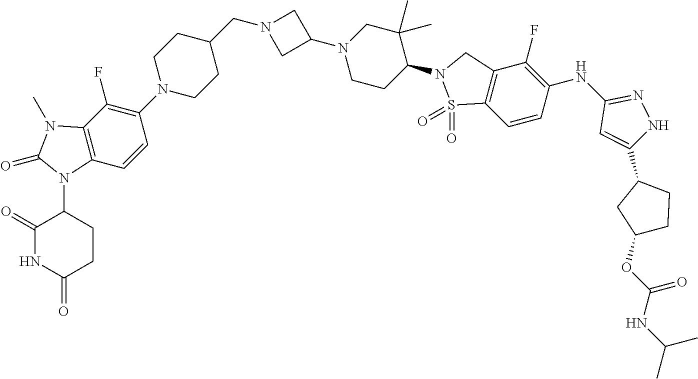 [SMILES: CC(C)NC(=O)O[C@H]1CC[C@@H](c2cc(Nc3ccc4c(c3F)CN([C@H]3CCN(C5CN(CC6CCN(c7ccc8c(c7F)n(C)c(=O)n8C7CCC(=O)NC7=O)CC6)C5)CC3(C)C)S4(=O)=O)n[nH]2)C1]

### Step 1: tert-butyl 3-((S)-4-(4-fluoro-5-((5-((1R,3S)-3-((isopropylcarbamoyl)oxy)cyclopentyl)-1H-pyrazol-3-yl)amino)-1,1-dioxidobenzo[d]isothiazol-2(3H)-yl)-3,3-dimethylpiperidin-1-yl)azetidine-1l-carboxylate

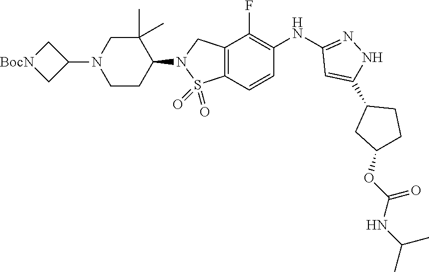 [SMILES: CC(C)NC(=O)O[C@H]1CC[C@@H](c2cc(Nc3ccc4c(c3F)CN([C@H]3CCN(C5CN(C(=O)OC(C)(C)C)C5)CC3(C)C)S4(=O)=O)n[nH]2)C1]

(1S,3R)-3-(3-((2-((S)-3,3-dimethylpiperidin-4-yl)-4-fluoro-1,1-dioxido-2,3-dihydrobenzo[d]isothiazol-5-yl)amino)-1H-pyrazol-5-yl)cyclopentyltert-butylcarbamateh (300 mg, 0.54 mmol) and tert-butyl 3-hydroxyazetidine-1-carboxylate (185 mg, 1.06 mmol) were dissolved in N,N-dimethylacetamide (7 mL) and stirred for 30 minutes. Sodium triacetoxyborohydride (229 mg, 1.08 mmol) was added, and the reaction mixture was stirred for 16 hours at room temperature. The resulting mixture was concentrated, and the residue was purified by column chromatography on silica gel, eluting with DCM/MeOH (10:1) to afford the product (300 mg, 80% yield). LC-MS (ESI): m/z [M+H]⁺=704.3.

### Step 2: (1S,3R)-3-(3-((2-((S)-1-(azetidin-3-yl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropylcarbamate

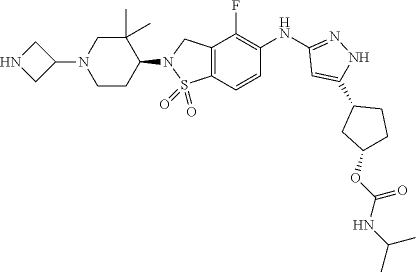 [SMILES: CC(C)NC(=O)O[C@H]1CC[C@@H](c2cc(Nc3ccc4c(c3F)CN([C@H]3CCN(C5CNC5)CC3(C)C)S4(=O)=O)n[nH]2)C1]

tert-butyl 3-((S)-4-(4-fluoro-5-((5-((1R,3S)-3-((isopropylcarbamoyl)oxy)cyclopentyl)-1H-pyrazol-3-yl)amino)-1,1-dioxidobenzo[d]isothiazol-2(3H)-yl)-3,3-dimethylpiperidin-1-yl)azetidine-1-carboxylate (300 mg, 0.43 mmol) in DCM was dissolved in 4M HCl (5 mL), the reaction mixture was stirred for 2 hours at room temperature. The resulting mixture was concentrated under reduce pressure. The resulting mixture was EA dilution adjusted PH to 7 with addition of TEA, a precipitated was washed with water, dried over in vacuum, to afford the product dried over in vacuum, to afford the product (252 mg, 100% yield). LC-MS (ESI): m/z [M+H]⁺=604.3.

### Step 3: (1S,3R)-3-(3-((2-((4S)-1-(1-((1-(1-(2,6-dioxopiperidin-3-yl)-4-fluoro-3-methyl-2-oxo-2,3-dihydro-1H-benzo[d]imidazol-5-yl)piperidin-4-yl)methyl)azetidin-3-yl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropylcarbamate

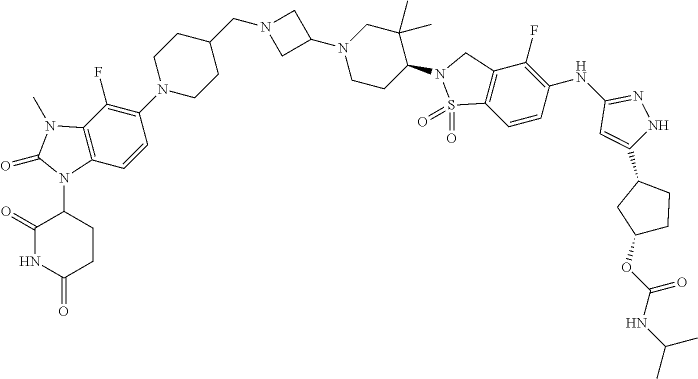 [SMILES: CC(C)NC(=O)O[C@H]1CC[C@@H](c2cc(Nc3ccc4c(c3F)CN([C@H]3CCN(C5CN(CC6CCN(c7ccc8c(c7F)n(C)c(=O)n8C7CCC(=O)NC7=O)CC6)C5)CC3(C)C)S4(=O)=O)n[nH]2)C1]

The titled compound was synthesized in the procedures similar to Example 37. EH NMR (500 MHz, DMSO) 11.99 (s, 1H), 11.10 (s, 1H), 8.77 (s, 1H), 8.30 (s, 1H), 7.54 (d, J=8.7 Hz, 1H), 6.94 (d, J 7.4 Hz, 1H), 6.85 (d, J 8.3 Hz, 1H), 6.74 (t, J 8.1 Hz, 1H), 5.84 (s, 1H), 5.33 (dd, J=12.8, 5.4 Hz, 1H), 4.99 (s, 1H), 4.54 (s, 2H), 3.64-3.54 (m, 1H), 3.47 (s, 3H), 3.28-3.20 (m, 3H), 3.10-3.02 (m, 1H), 2.94-2.84 (m, 2H), 2.73-2.65 (m, 3H), 2.61-2.54 (m, 8H), 2.43-2.40 (m, 1H), 2.20-2.28 (m, 2H), 2.07-1.96 (m, 3H), 1.94-1.82 (m, 4H), 1.80-1.66 (m, 4H), 1.64-1.50 (m, 2H), 1.48-1.34 (m, 2H), 1.12 (s, 3H), 1.06-0.99 (m, 6H), 0.95 (s, 3H). LC-MS (ESI): m/z [M+H]⁺=976.4;

### Example 225: (1S,3R)-3-(3-((2-((4S)-1-(1-(1-(1-(2,6-dioxopiperidin-3-yl)-4-fluoro-3-methyl-2-oxo-2,3-dihydro-1H-benzo[d]imidazol-5-yl)piperidin-4-yl)azetidin-3-yl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropylcarbamate

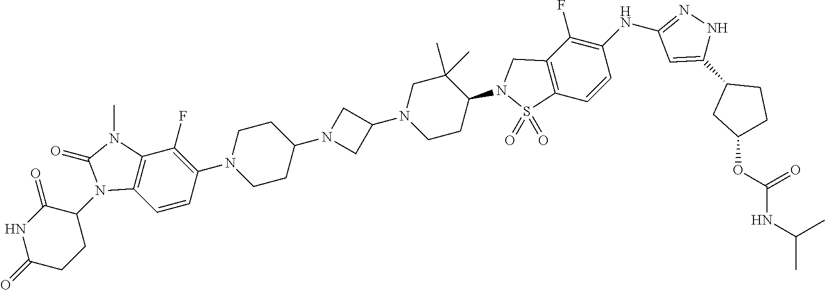 [SMILES: CC(C)NC(=O)O[C@H]1CC[C@@H](c2cc(Nc3ccc4c(c3F)CN([C@H]3CCN(C5CN(C6CCN(c7ccc8c(c7F)n(C)c(=O)n8C7CCC(=O)NC7=O)CC6)C5)CC3(C)C)S4(=O)=O)n[nH]2)C1]

The titled compound was synthesized in the procedures similar to Example 224. ¹H NMR (500 MHz, DMSO) δ 11.99 (s, 1H), 11.10 (s, 1H), 8.77 (s, 1H), 8.30 (s, 1H), 7.54 (d, J=8.7 Hz, 1H), 6.94 (d, J=7.4 Hz, 1H), 6.85 (d, J=8.3 Hz, 1H), 6.74 (t, J=8.1 Hz, 1H), 5.84 (s, 1H), 5.33 (dd, J=12.8, 5.4 Hz, 1H), 4.99 (s, 1H), 4.54 (s, 2H), 3.64-3.54 (m, 1H), 3.47 (s, 3H), 3.28-3.20 (m, 3H), 3.10-3.02 (m, 1H), 2.94-2.84 (m, 2H), 2.73-2.65 (m, 3H), 2.61-2.54 (m, 6H), 2.43-2.40 (m, 1H), 2.20-2.28 (m, 2H), 2.07-1.96 (m, 3H), 1.94-1.82 (m, 4H), 1.80-1.66 (m, 4H), 1.64-1.50 (m, 2H), 1.48-1.34 (m, 2H), 1.12 (s, 3H), 1.06-0.99 (m, 6H), 0.95 (s, 3H). LC-MS (ESI): m/z [M+H]⁺=962.4;

### Example 226: (1S,3R)-3-(3-((2-((S)-1-((1-(1-((S)-2,6-dioxopiperidin-3-yl)-7-fluoro-3-methyl-2-oxo-2,3-dihydro-1H-benzo[d]imidazol-5-yl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropyl carbamate

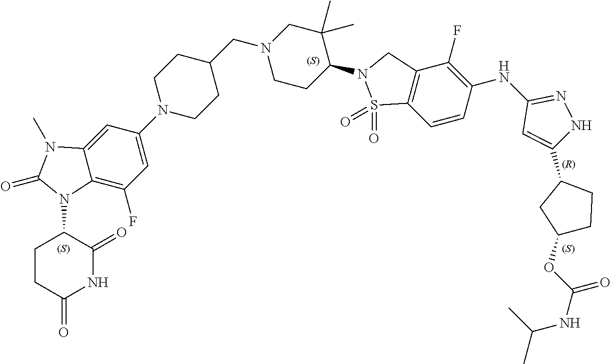

The titled compound was synthesized in the procedures similar to Example 41 in a racemate form, which was further separated by chiral HPLC to give Example 226 (100% ee); Retention time: 9.985 min. ¹H NMR (500 MHz, DMSO) δ 11.99 (s, 1H), 11.07 (s, 1H), 8.77 (s, 1H), 8.31 (t, J=7.9 Hz, 1H), 7.54 (d, J=8.6 Hz, 1H), 6.94 (d, J=7.3 Hz, 1H), 6.68 (s, 1H), 6.52 (d, J=14.5 Hz, 1H), 5.83 (s, 1H), 5.40 (s, 1H), 5.00 (s, 1H), 4.58-4.50 (m, 2H), 3.67 (s, 2H), 3.62-3.53 (m, 1H), 3.33 (s, 3H), 3.22 (dd, J=12.2, 3.8 Hz, 1H), 3.10-2.90 (m, 3H), 2.69-2.56 (m, 4H), 2.24-1.88 (m, 8H), 1.84-1.57 (m, 7H), 1.50 (d, J=9.5 Hz, 1H), 1.27-1.18 (m, 2H), 1.15-1.10 (m, 3H), 1.08-0.99 (m, 6H), 0.94 (s, 3H). LC-MS (ESI): m/z [M+H]+=921.22.

Chiral analytical method: Column: YMC Amylose-SA 4.6*250 mm 5 m Mobile phase: A for MtBE (0.1%2M NH3 MeOH) (%) and B for MeOH:DCM=50:50 (v/v); Gradient: Mobile Phase A: Mobile Phase B=30:70 (v/v); HPLC Equipment: Agilent 1260 Infinity II (ARD_LC-808); Column temperature: 25° C.

Chiral Prep-HPLC Condition: YMC Amylose-SA 4.6*250 mm 5 m Mobile phase: A for MtBE (0.1%2M NH3 MeOH)(%) and B for MeOH:DCM=50:50; Gradient: Mobile Phase A: Mobile Phase B=30:70 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 227: (1S,3R)-3-(3-((2-((4S)-1-((1-(1-(2,6-dioxopiperidin-3-yl)-2-oxo-1,2,3,4-tetrahydro-5-oxa-1,2a-diazaacenaphthylen-6-yl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropylcarbamate

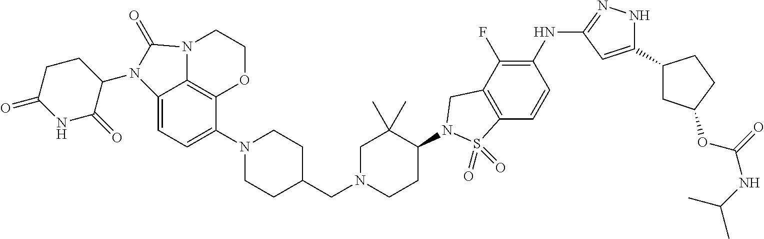 [SMILES: CC(C)NC(=O)O[C@H]1CC[C@@H](c2cc(Nc3ccc4c(c3F)CN([C@H]3CCN(CC5CCN(c6ccc7c8c6OCCn8c(=O)n7C6CCC(=O)NC6=O)CC5)CC3(C)C)S4(=O)=O)n[nH]2)C1]

### Step 1: 1-(2,6-bis(benzyloxy)pyridin-3-yl)-6-(4-(dimethoxymethyl) piperidin-1-yl)-3,4-dihydro-5-oxa-1,2a-diazaacenaphthylen-2(1H)-one

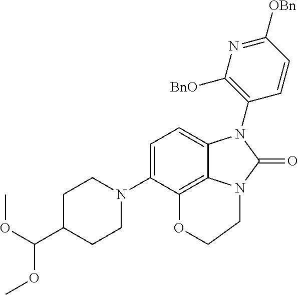 [SMILES: COC(OC)C1CCN(c2ccc3c4c2OCCn4c(=O)n3-c2ccc(OCc3ccccc3)nc2OCc2ccccc2)CC1]

To a solution of 1-(2,6-bis(benzyloxy)pyridin-3-yl)-6-bromo-3,4-dihydro-5-oxa-1,2a-diazaacenaphthylen-2(1H)-one (PH-BGUS-H-A636-6) (1.5 g, 2.8 mmol) in dioxane (20 mL) were added 4-(dimethoxymethyl) piperidine (614.4 mg, 3.9 mmol), Pd-PEPPSI-IPentCl 2-methylpyridine (o-picoline) (115.9 mg, 0.1 mmol) and Cs₂CO₃ (2.7 g, 8.4 mmol). The resulting solution was stirred for 3 h at 100° C. under nitrogen atmosphere. The resulting mixture was concentrated under vacuum. The residue was purified by combi-flash (EtOAc/PE=80%) to give the product (1.4 g, 82.4%) as yellow solid. MS (ESI) m/e [M+1]⁺=623.

### Step 2: 3-(6-(4-(dimethoxymethyl) piperidin-1-yl)-2-oxo-3,4-dihydro-5-oxa-1,2a-diazaacenaphthylen-1(2H)-yl) piperidine-2,6-dione

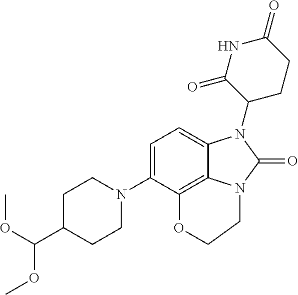 [SMILES: COC(OC)C1CCN(c2ccc3c4c2OCCn4c(=O)n3C2CCC(=O)NC2=O)CC1]

To a solution of 1-(2,6-bis(benzyloxy)pyridin-3-yl)-6-(4-(dimethoxymethyl) piperidin-1-yl)-3,4-dihydro-5-oxa-1,2a-diazaacenaphthylen-2(1H)-one (1.4 g, 2.2 mmol) in THF (20 mL) was added Pd/C (1.4 g, 10% wt). The resulting solution was stirred for 12 h at 50° C. under H₂ atmosphere. After filtration, the filtrate was concentrated under vacuum. The residue was the product (954.1 mg, 97.7%) as white solid. MS (ESI) m/e [M+1]⁺=445.

### Step 3: (1S,3R)-3-(3-((2-((4S)-1-((1-(1-(2,6-dioxopiperidin-3-yl)-2-oxo-1,2,3,4-tetrahydro-5-oxa-1,2a-diazaacenaphthylen-6-yl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropylcarbamate

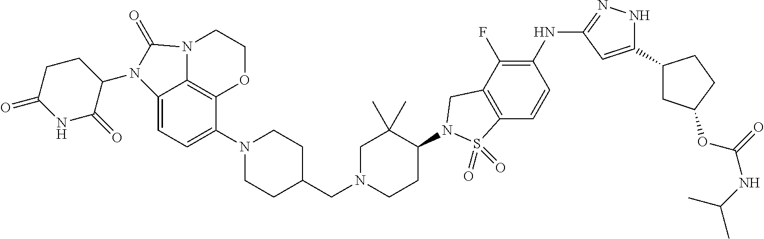 [SMILES: CC(C)NC(=O)O[C@H]1CC[C@@H](c2cc(Nc3ccc4c(c3F)CN([C@H]3CCN(CC5CCN(c6ccc7c8c6OCCn8c(=O)n7C6CCC(=O)NC6=O)CC5)CC3(C)C)S4(=O)=O)n[nH]2)C1]

The titled compound was synthesized in the procedures similar to Example 109. LC-MS (ESI): m/z [M+H]⁺=931.70; ¹H NMR (500 MHz, DMSO) δ=11.99 (s, 1H), 11.07 (s, 1H), 8.76 (s, 1H), 8.30 (s, 1H), 7.54 (d, J=8.6, 1H), 6.94 (d, J=7.3, 1H), 6.59 (d, J=8.4, 1H), 6.53 (d, J=8.4, 1H), 5.83 (s, 1H), 5.27 (dd, J=12.9, 5.4, 1H), 5.00 (s, 1H), 4.54 (s, 2H), 4.38 (s, 2H), 3.91 (s, 2H), 3.61-3.55 (m, 1H), 3.24-3.21 (m, 1H), 3.10-3.02 (m, 1H), 2.98-2.87 (m, 2H), 2.70-2.66 (m, 1H), 2.65-2.61 (m, 2H), 2.57 (m, 3H), 2.45 (m, 1H), 2.14 (m, 3H), 2.06-1.96 (m, 3H), 1.95-1.87 (m, 1H), 1.84-1.69 (m, 5H), 1.59 (m, 2H), 1.50 (m, 1H), 1.26 (m, 3H), 1.12 (s, 3H), 1.03 (m, 6H), 0.94 (s, 3H).

### Example 228: (1S,3R)-3-(3-((2-((4S)-1-((1-(1-(2,6-dioxopiperidin-3-yl)-2-oxo-1,2,5,6-tetrahydro-4H-imidazo[4,5,1-ij]quinolin-7-yl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropylcarbamate

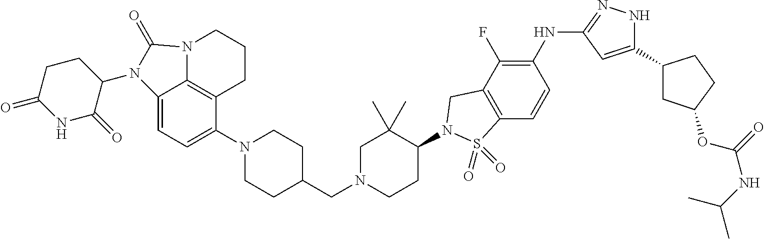 [SMILES: CC(C)NC(=O)O[C@H]1CC[C@@H](c2cc(Nc3ccc4c(c3F)CN([C@H]3CCN(CC5CCN(c6ccc7c8c6CCCn8c(=O)n7C6CCC(=O)NC6=O)CC5)CC3(C)C)S4(=O)=O)n[nH]2)C1]

The titled compound was synthesized in the procedures similar to Example 180. LC-MS (ESI): m/z [M+H]⁺=929.73; ¹H NMR (500 MHz, DMSO) δ=11.99 (s, 1H), 11.05 (s, 1H), 8.76 (s, 1H), 8.31 (t, J=7.8, 1H), 7.54 (d, J=8.7, 1H), 6.94 (d, J=7.6, 1H), 6.82 (d, J=8.3, 1H), 6.62 (d, J=8.4, 1H), 5.83 (s, 1H), 5.27 (dd, J=13.0, 5.3, 1H), 5.00 (s, 1H), 4.56 (d, J=15.4, 2H), 3.72 (m, 2H), 3.58 (m, 1H), 3.25-3.21 (m, 1H), 3.08 (m, 3H), 2.96 (m, 1H), 2.94-2.87 (m, 1H), 2.72 (m, 3H), 2.65-2.62 (m, 2H), 2.59 (m, 2H), 2.47-2.42 (m, 1H), 2.24-2.16 (m, 2H), 2.16-2.10 (m, 1H), 2.04-1.94 (m, 5H), 1.91 (m, 1H), 1.81 (m, 3H), 1.71 (m, 2H), 1.59 (m, 2H), 1.51 (m, 1H), 1.24 (m, 2H), 1.13 (s, 3H), 1.03 (m, 6H), 0.94 (s, 3H).

### Example 229: (1S,3R)-3-(3-((2-((S)-1-((1-(1-((S)-2,6-dioxopiperidin-3-yl)-4,7-difluoro-3-methyl-2-oxo-2,3-dihydro-1H-benzo[d]imidazol-5-yl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropylcarbamate

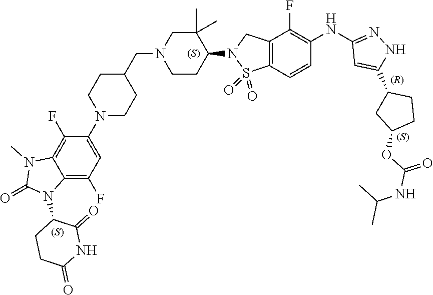

The titled compound was synthesized in the procedures similar to Example 55 in a racemate form, which was further separated by chiral HPLC to give Example 229 (100% ee); Retention time: 5.79 min. ¹H NMR (500 MHz, DMSO) δ 11.98 (s, 1H), 11.11 (s, 1H), 8.76 (s, 1H), 8.31 (t, J=8.0 Hz, 1H), 7.54 (d, J=8.6 Hz, 1H), 6.94 (d, J=7.8 Hz, 1H), 6.66 (s, 1H), 5.83 (s, 1H), 5.47 (s, 1H), 5.00 (s, 1H), 4.54 (s, 2H), 3.60-3.59 (m, 1H), 3.50 (s, 3H), 3.29-3.17 (m, 3H), 3.06 (s, 1H), 2.96 (s, 2H), 2.68-2.53 (m, 6H), 2.24-2.07 (m, 3H), 2.06-1.96 (m, 2H), 1.91 (s, 1H), 1.84-1.80 (m, 3H), 1.71 (s, 2H), 1.61 (s, 2H), 1.51-1.49 (m, 1H), 1.33-1.22 (m, 3H), 1.12 (s, 3H), 1.03-1.02 (m, 6H), 0.94 (s, 3H). LC-MS (ESI): m/z [M+H]+=939.59.

Chiral analytical method: Column: I-Cellulose-5, 4.6 mm×250 mm, 5 um; Mobile phase: A for Hexane and B for EtOH (0.1% 2M NH₃ MeOH); Gradient: Mobile Phase A: Mobile Phase B=30:70 (v/v); HPLC Equipment: HPLC-Agilent, Back pressure: 100 bar; Column temperature: 35° C.

Chiral Prep-HPLC Condition: Column: I-Cellulose-5, 21.2 mm×250 mm, 5 um; Mobile phase: A for Hexane and B for EtOH (0.2% 2M NH₃ MeOH); Gradient: Mobile Phase A: Mobile Phase B=30:70 (v/v); Flow Rate: 20 mL/min, Wave Length: UV 200 nm and 270 nm, Prep-HPLC Equipment: Prep-HPLC-Gilson, Back pressure: 100 bar; Column temperature: 25° C.

### Example 230: (1S,3R)-3-(3-((2-((4S)-1-((1-(1-(2,6-dioxopiperidin-3-yl)-3-methyl-2-oxo-2,3-dihydro-1H-benzo[d]imidazol-5-yl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropylcarbamate

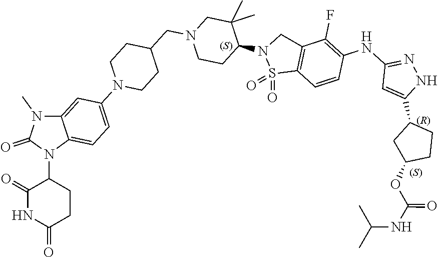

The titled compound was synthesized in the procedures similar to Example 19. ¹H NMR (500 MHz, DMSO) δ 11.99 (s, 1H), 11.06 (s, 1H), 8.77 (s, 1H), 8.30 (s, 1H), 7.54 (d, J=8.6 Hz, 1H), 6.92 (d, J=8.7 Hz, 2H), 6.83 (s, 1H), 6.64 (d, J=8.4 Hz, 1H), 5.83 (s, 1H), 5.28 (dd, J=12.8, 5.4 Hz, 1H), 5.00 (s, 1H), 4.55 (s, 2H), 3.59 (s, 3H), 3.30 (s, 3H), 3.23 (dd, J=12.1, 3.7 Hz, 1H), 3.11-3.04 (m, 1H), 2.99-2.84 (m, 2H), 2.70-2.55 (m, 6H), 2.20-1.50 (m, 15H), 1.31-1.21 (m, 2H), 1.13 (s, 3H), 1.06-1.00 (m, 6H), 0.94 (s, 3H). LC-MS (ESI): m/z [M+H]⁺=903.54.

### Example 231: (1S,3R)-3-(3-((2-((4R)-1-((1-(1-(2,6-dioxopiperidin-3-yl)-3-methyl-2-oxo-2,3-dihydro-1H-benzo[d]imidazol-5-yl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropyl carbamate

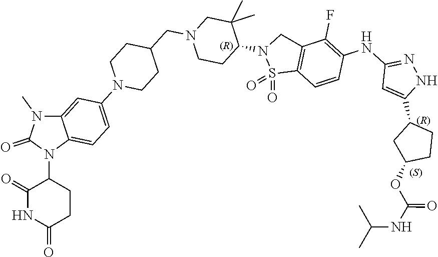

The titled compound was synthesized in the procedures similar to Example 19. ¹H NMR (500 MHz, DMSO) δ 11.99 (s, 1H), 11.06 (s, 1H), 8.77 (s, 1H), 8.30 (s, 1H), 7.54 (d, J=8.6 Hz, 1H), 6.92 (d, J=8.7 Hz, 2H), 6.83 (s, 1H), 6.64 (d, J=8.4 Hz, 1H), 5.83 (s, 1H), 5.28 (dd, J=12.8, 5.4 Hz, 1H), 5.00 (s, 1H), 4.55 (s, 2H), 3.59 (s, 3H), 3.30 (s, 3H), 3.23 (dd, J=12.1, 3.7 Hz, 1H), 3.11-3.04 (m, 1H), 2.99-2.84 (m, 2H), 2.70-2.55 (m, 6H), 2.20-1.50 (m, 15H), 1.31-1.21 (m, 2H), 1.13 (s, 3H), 1.06-1.00 (m, 6H), 0.94 (s, 3H). LC-MS (ESI): m/z [M+H]⁺=903.41.

### Example 232: (1S,3R)-3-(3-((2-((R)-1-((1-(1-((S)-2,6-dioxopiperidin-3-yl)-7-fluoro-3-methyl-2-oxo-2,3-dihydro-1H-benzo[d]imidazol-5-yl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropylcarbamate

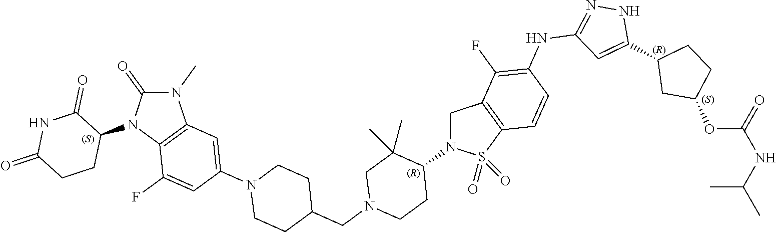

### Step 1: 3-(2,6-bis(benzyloxy)pyridin-3-yl)-6-(4-(dimethoxymethyl)piperidin-1-yl)-4-fluoro-1-methyl-1,3-dihydro-2H-benzo[d]imidazol-2-one

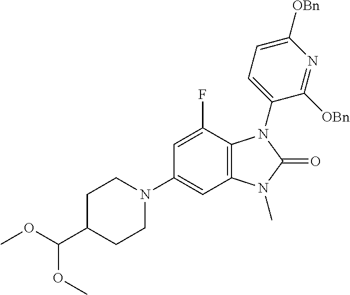 [SMILES: COC(OC)C1CCN(c2cc(F)c3c(c2)n(C)c(=O)n3-c2ccc(OCc3ccccc3)nc2OCc2ccccc2)CC1]

To a mixture of 3-(2,6-bis(benzyloxy)pyridin-3-yl)-6-bromo-4-fluoro-1-methyl-1,3-dihydro-2H-benzo[d]imidazol-2-one (5 g, 9.38 mmol) and 4-(dimethoxymethyl)piperidine (2.24 g, 14.07 mmol) in andydrous 1,4-dioxane (10 mL) was added PEPPSI (790 mg, 0.94 mmol) and Cs₂CO₃ (9.17 g, 28.14 mmol). The reaction mixture was stirred under nitrogen atmosphere at 100° C. for 16 h. The mixture was filtered, and the filtrate was concentrated under reduced pressure. The residue was purified by silica gel column chromatography, eluting with PE/EA (1:9) to afford the product (4.5 g, 78% yield). LC-MS (ESI): m/z [M+H]⁺=552.2.

### Step 2: 3-(5-(4-(dimethoxymethyl)piperidin-1-yl)-7-fluoro-3-methyl-2-oxo-2,3-dihydro-1H-benzo[d]imidazol-1-yl)piperidine-2,6-dione

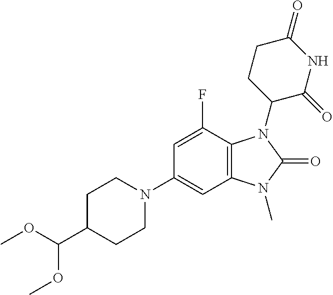 [SMILES: COC(OC)C1CCN(c2cc(F)c3c(c2)n(C)c(=O)n3C2CCC(=O)NC2=O)CC1]

To a solution of 3-(2,6-bis(benzyloxy)pyridin-3-yl)-6-(4-(dimethoxymethyl)piperidin-1-yl)-4-fluoro-1-methyl-1,3-dihydro-2H-benzo[d]imidazol-2-one (4.5 g, 7.35 mmol) in THF (15 mL) was added Pd/C (10%, wet, 3 g). The suspension was degassed and purged with hydrogen 3 times. The resulting mixture was stirred at 50° C. under a hydrogen balloon for 16 h. The mixture was filtered, and the filter cake was washed with DCM:MeOH (200 mL×3). The filtrate was concentrated under reduced pressure to afford the product (3 g, 90% yield). LC-MS (ESI): m/z [M+H]⁺=435.3

### Step 3: 1-(1-(2,6-dioxopiperidin-3-yl)-7-fluoro-3-methyl-2-oxo-2,3-dihydro-1H-benzo[d]imidazol-5-yl)piperidine-4-carbaldehyde

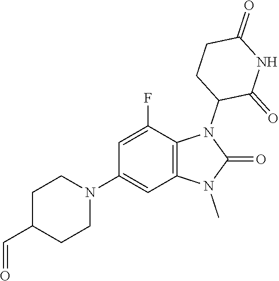 [SMILES: Cn1c(=O)n(C2CCC(=O)NC2=O)c2c(F)cc(N3CCC(C=O)CC3)cc21]

To a solution of 3-(5-(4-(dimethoxymethyl)piperidin-1-yl)-7-fluoro-3-methyl-2-oxo-2,3-dihydro-1H-benzo[d]imidazol-1-yl)piperidine-2,6-dione (3 g, 6.90 mmol) in FA (20 mL). The reaction mixture was stirred for 1 hours at room temperature. The resulting mixture was concentrated to afford the product (2.68 g, 100% yield). LC-MS (ESI): m/z [M+H]⁺=389.20.

### Step 4: (1S,3R)-3-(3-((2-((R)-1-((1-(1-((S)-2,6-dioxopiperidin-3-yl)-7-fluoro-3-methyl-2-oxo-2,3-dihydro-1H-benzo[d]imidazol-5-yl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropylcarbamate

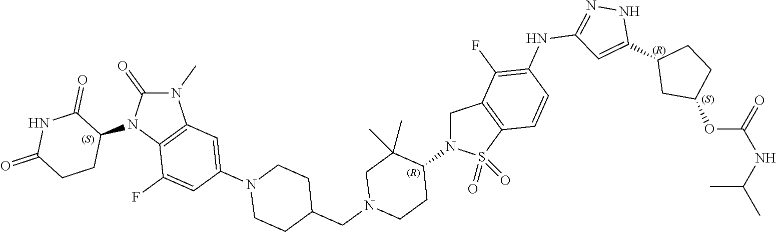

To a solution of (1S,3R)-3-(3-((2-((R)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropyl carbamate (30 mg, 0.055 mmol) and 1-(1-(2,6-dioxopiperidin-3-yl)-7-fluoro-3-methyl-2-oxo-2,3-dihydro-1H-benzo[d]imidazol-5-yl)piperidine-4-carbaldehyde (32 mg, 0.082 mmol) in DMA (1.5 mL) was stirred for 30 min, then STAB (23 mg, 0.109 mmol) was added. the reaction mixture was stirred for 16 hours at room temperature. The resulting mixture was concentrated under reduce pressure, the residue was purified by column chromatography on silica gel, eluting with DCM/MeOH (10:1) to afford the product (20 mg). The residue 20 mg was purified by prep HPLC (Waters XSelect C18: RD-CO-094 column, eluting with a gradient of acetonitrile/water containing 0.1% FA, 20%-40%) to afford the product (9 mg, 17.6%). Then further separated by chiral HPLC to give Example 232 (99.26% ee); Retention time: 7.294 min. ¹H NMR (500 MHz, DMSO) δ 12.00 (d, J=10.2 Hz, 1H), 11.07 (s, 1H), 8.79 (d, J=22.2 Hz, 1H), 8.62 (s, OH), 8.38-8.23 (m, 1H), 7.56 (dd, J=21.5, 8.6 Hz, 1H), 6.94 (d, J=7.2 Hz, 1H), 6.69 (d, J=7.1 Hz, 1H), 6.54 (t, J=15.1 Hz, 1H), 5.84 (s, 1H), 5.41 (s, 1H), 5.00 (s, 1H), 4.56 (d, J=21.0 Hz, 2H), 3.75-3.55 (m, H), 3.34 (s, 3H), 3.23 (d, J=11.6 Hz, 1H), 3.13-2.89 (m, 4H), 2.79-2.57 (m, 6H), 2.22-1.97 (m, 5H), 1.91 (s, 1H), 1.85-1.61 (m, 6H), 1.36 (d, J=10.3 Hz, 1H), 1.24 (s, 2H), 1.15-0.85 (m, 12H). LC-MS (ESI): m/z [M+H]⁺=921.60.

Chiral analytical method: Column: YMC Amylose-SA 4.6*250 mm 5 m Mobile phase: A for MtBE (0.1%2M NH3 MeOH) (%) and B for MeOH:DCM=50:50 (v/v); Gradient: Mobile Phase A: Mobile Phase B=40:60 (v/v); HPLC Equipment: Agilent 1260 Infinity II (ARD_LC-808); Column temperature: 25° C.

Chiral Prep-HPLC Condition: YMC Amylose-SA 4.6*250 mm 5 m Mobile phase: A for MtBE (0.1%2M NH3 MeOH)(%) and B for MeOH:DCM=50:50; Gradient: Mobile Phase A: Mobile Phase B=40:60 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 233: (1S,3R)-3-(3-((2-((4S)-1-((1-(1-(2,6-dioxopiperidin-3-yl)-3-methyl-2-oxo-2,3-dihydro-1H-benzo[d]imidazol-5-yl)azetidin-3-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropylcarbamate

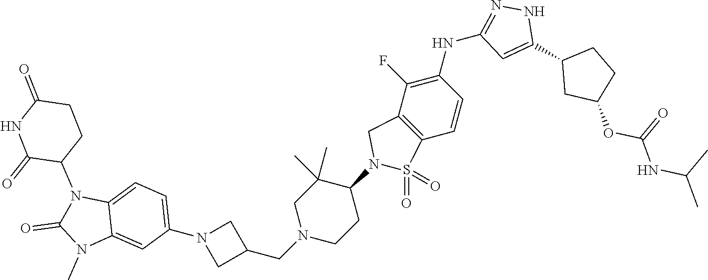 [SMILES: CC(C)NC(=O)O[C@H]1CC[C@@H](c2cc(Nc3ccc4c(c3F)CN([C@H]3CCN(CC5CN(c6ccc7c(c6)n(C)c(=O)n7C6CCC(=O)NC6=O)C5)CC3(C)C)S4(=O)=O)n[nH]2)C1]

The titled compound was synthesized in the procedures similar to Example 113. ¹H NMR (500 MHz, DMSO) δ 12.01 (s, 1H), 11.06 (s, 1H), 8.96 (s, 1H), 8.81 (s, 1H), 8.33 (s, 1H), 7.58 (d, J=8.8 Hz, 1H), 6.93 (d, J=8.4 Hz, 2H), 6.35 (s, 1H), 6.14 (d, J=9.1 Hz, 1H), 5.84 (s, 1H), 5.28 (dd, J=12.3, 5.4 Hz, 1H), 5.06-4.96 (m, 1H), 4.59 (s, 2H), 4.10-4.07 (m, 2H), 3.75 (d, J=14.6 Hz, 1H), 3.63-3.52 (m, 4H), 3.51-3.45 (m, 2H), 3.29 (s, 3H), 3.25-3.12 (m, 4H), 3.12-3.03 (m, 1H), 2.96-2.84 (m, 2H), 2.47-2.42 (m, 1H), 2.35-2.27 (m, 1H), 2.05-1.89 (m, 3H), 1.86-1.80 (m, 1H), 1.78-1.65 (m, 2H), 1.65-1.56 (m, 1H), 1.22 (s, 3H), 1.07-1.00 (m, 9H). LC-MS (ESI): m/z [M+H]⁺=875.4;

### Example 234: (1S,3R)-3-(3-((2-((4S)-1-((1-(1-((S)-2,6-dioxopiperidin-3-yl)-4-fluoro-3-methyl-2-oxo-2,3-dihydro-1H-benzo[d]imidazol-5-yl)-3,3-difluoropiperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropylcarbamate

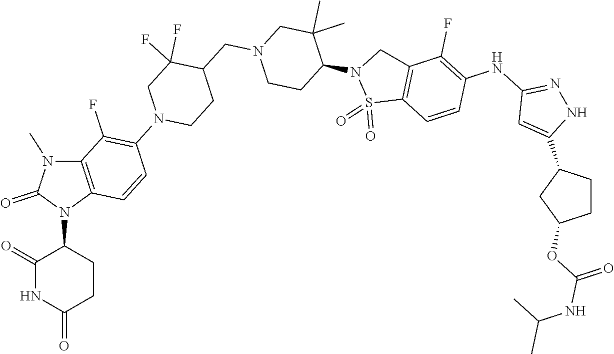 [SMILES: CC(C)NC(=O)O[C@H]1CC[C@@H](c2cc(Nc3ccc4c(c3F)CN([C@H]3CCN(CC5CCN(c6ccc7c(c6F)n(C)c(=O)n7[C@H]6CCC(=O)NC6=O)CC5(F)F)CC3(C)C)S4(=O)=O)n[nH]2)C1]

### Step 1: 1-(1-((S)-2,6-dioxopiperidin-3-yl)-4-fluoro-3-methyl-2-oxo-2,3-dihydro-1H-benzo[d]imidazol-5-yl)-3,3-difluoropiperidine-4-carbaldehyde

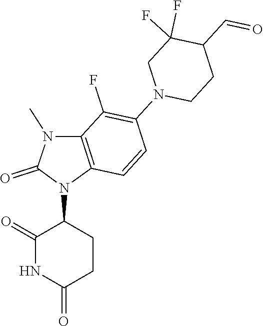 [SMILES: Cn1c(=O)n([C@H]2CCC(=O)NC2=O)c2ccc(N3CCC(C=O)C(F)(F)C3)c(F)c21]

The titled compound was synthesized in the procedures similar to Example 200. LC-MS (ESI): m/z [M+H]⁺=425.4;

### Step 2: (1S,3R)-3-(3-((2-((4S)-1-((1-(1-((S)-2,6-dioxopiperidin-3-yl)-4-fluoro-3-methyl-2-oxo-2,3-dihydro-1H-benzo[d]imidazol-5-yl)-3,3-difluoropiperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropylcarbamate

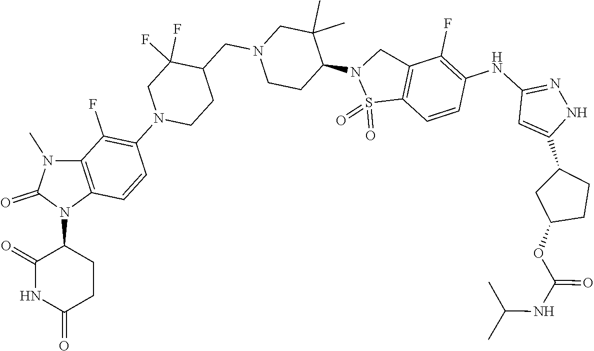 [SMILES: CC(C)NC(=O)O[C@H]1CC[C@@H](c2cc(Nc3ccc4c(c3F)CN([C@H]3CCN(CC5CCN(c6ccc7c(c6F)n(C)c(=O)n7[C@H]6CCC(=O)NC6=O)CC5(F)F)CC3(C)C)S4(=O)=O)n[nH]2)C1]

The titled compound was synthesized in the procedures similar to Example 37. LC-MS (ESI): m/z [M+H]⁺=957.48; ¹H NMR (500 MHz, DMSO) δ=12.00 (s, 1H), 11.09 (s, 1H), 9.09-8.79 (m, 1H), 8.33 (s, 1H), 7.58 (m, 1H), 6.94 (d, J=6.2, 1H), 6.80 (s, 1H), 6.64 (s, 1H), 5.84 (s, 1H), 5.42 (s, 1H), 5.00 (s, 1H), 4.57 (m, 2H), 3.97 (m, 1H), 3.75 (m, 2H), 3.58 (m, 1H), 3.25-3.21 (m, 1H), 3.07 (m, 3H), 2.85 (m, 1H), 2.54 (m, 5H), 2.46-2.45 (m, 1H), 2.20 (m, 2H), 2.13-1.97 (m, 4H), 1.88 (m, 2H), 1.72 (m, 3H), 1.60 (m, 1H), 1.53 (m, 1H), 1.23 (m, 4H), 1.14 (m, 2H), 1.06 (m, 2H), 1.03 (m, 6H), 0.95 (m, 1H).

### Example 235: (1S,3R)-3-(3-((2-((S)-1-((1-(1-((S)-2,6-dioxopiperidin-3-yl)-8-fluoro-2-oxo-1,2,3,4-tetrahydro-5-oxa-1,2a-diazaacenaphthylen-6-yl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropylcarbamate

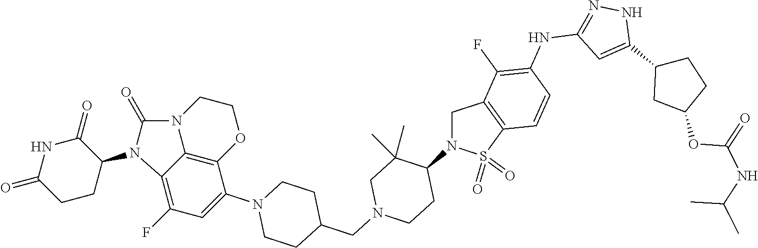 [SMILES: CC(C)NC(=O)O[C@H]1CC[C@@H](c2cc(Nc3ccc4c(c3F)CN([C@H]3CCN(CC5CCN(c6cc(F)c7c8c6OCCn8c(=O)n7[C@H]6CCC(=O)NC6=O)CC5)CC3(C)C)S4(=O)=O)n[nH]2)C1]

The titled compound was synthesized in the procedures similar to Example 186. ¹H NMR (500 MHz, DMSO) δ 11.98 (s, 1H), 11.10 (s, 1H), 8.77 (s, 1H), 8.31 (t, J=8.1 Hz, 1H), 7.54 (d, J=8.6 Hz, 1H), 6.93 (s, 1H), 6.40 (d, J=13.5 Hz, 1H), 5.83 (s, 1H), 5.38 (s, 1H), 5.06-4.96 (m, 1H), 4.54 (s, 2H), 4.40-4.33 (m, 2H), 3.96-3.90 (m, 2H), 3.65-3.53-3.54 (m, 1H), 3.46-3.36 (m, 4H), 3.24-3.20 (m, 1H), 3.10-2.92 (m, 3H), 2.61-2.57 (m, 5H), 2.21-2.14 (m, 2H), 2.13-2.07 (m, 2H), 2.06-1.85 (m, 3H), 1.84-1.66 (m, 5H), 1.64-1.55 (m, 2H), 1.53-1.48 (m, 1H), 1.31-1.20 (m, 2H), 1.12 (s, 3H), 1.03 (d, J=6.5 Hz, 6H), 0.93 (s, 3H). LC-MS (ESI): m/z [M+H]⁺=949.4;

### Example 236: (1S,3R)-3-(3-((2-((S)-1′-(1-((S)-2,6-dioxopiperidin-3-yl)-8-fluoro-2-oxo-1,2,3,4-tetrahydro-5-oxa-1,2a-diazaacenaphthylen-6-yl)-3,3-dimethyl-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

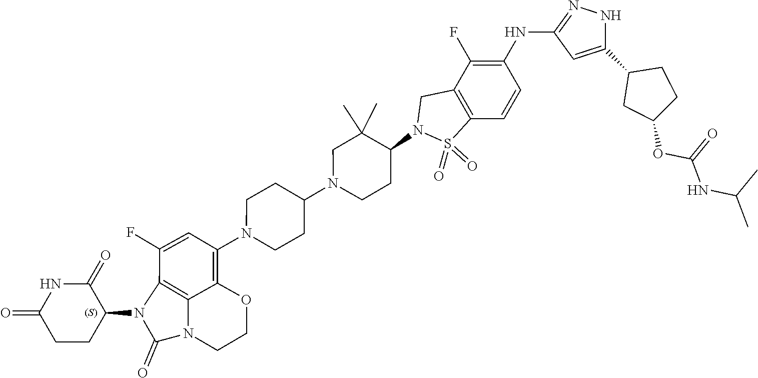

The racemic compound was synthesized in the procedures similar to Example 187, which was further separated by Chiral Prep-HPLC to afford Example 236 (100% ee); Retention time: 9.13 min. ¹H NMR (500 MHz, DMSO-d₆) δ 11.99 (s, 1H), 11.11 (s, 1H), 8.76 (s, 1H), 8.31 (t, J=5 Hz, 1H), 7.53 (t, J=10 Hz, 1H), 6.97-6.92 (m, 1H), 6.43-6.36 (m, 1H), 5.83 (s, 1H), 5.45-5.30 (m, 1H), 5.04-4.96 (m, 1H), 4.53 (s, 2H), 4.41-4.32 (m, 2H), 3.99-3.87 (m, 2H), 3.63-3.52 (m, 1H), 3.50-3.41 (m, 2H), 3.22-3.16 (m, 1H), 3.12-2.93 (m, 3H), 2.62-2.56 (m, 4H), 2.46-2.44 (m, 1H), 2.39-2.26 (m, 2H), 2.16-1.97 (m, 4H), 1.97-1.87 (m, 1H), 1.83-1.66 (m, 4H), 1.65-1.51 (m, 4H), 1.09 (s, 3H), 1.07-1.00 (m, 6H), 0.94 (s, 3H). LC-MS (ESI): m/z [M+H]⁺=935.

Chiral analytical method: Amylose-SA, 4.6 mm×250 mm, 5 um; Mobile phase: A for MtBE (0.1% 2M NH3 MeOH) and B for DCM:MeOH (50:50); Gradient: Mobile Phase A: Mobile Phase B=20:80 (v/v); HPLC Equipment: HPLC-Agilent, Back pressure: 100 bar; Column temperature: 35° C.

Chiral Prep-HPLC Condition: Column: Cellulose-SA, 250 mm×20 mm, 5 um; Mobile phase: A for MtBE (0.1% 2M NH3 MeOH) and B for DCM:MeOH (50:50); Gradient: Mobile Phase A: Mobile Phase B=20:80 (v/v); Flow Rate: 20 mL/min, Wave Length: UV 200 nm and 270 nm, Prep-HPLC Equipment: Prep-HPLC-Gilson, Back pressure: 100 bar; Column temperature: 25° C.

### Example 237: (1S,3R)-3-(3-((2-((S)-1-((1-(1-((R)-2,6-dioxopiperidin-3-yl)-7-fluoro-3,4-dimethyl-2-oxo-2,3-dihydro-1H-benzo[d]imidazol-5-yl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropyl carbamate

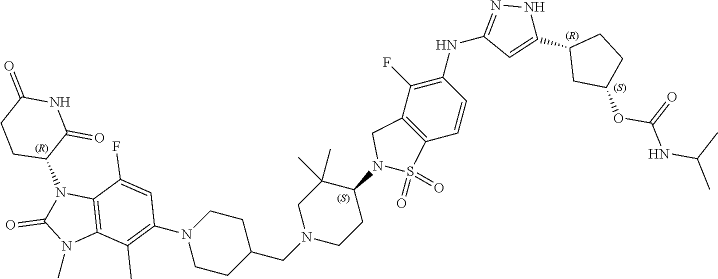

### Step 1: (E)-N-(3-bromo-5-fluoro-2-methylphenyl)-2-(hydroxyimino)acetamide

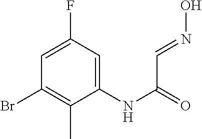

To a solution of sodium sulfate (167.07 g, 1181.6 mmol), hydroxylamine hydrochloride (35.76 g, 517.12 mmol), and chloral (32.5 g, 221.55 mmol) was dissolved in water (750 mL). To the reaction mixture was added a solution of 3-bromo-5-fluoro-2-methylaniline (30 g, 147.7 mmol) in a mixture of water (810 mL), ethanol (105 mL), and concentrated HCl (25.8 mL). The reaction mixture was stirred overnight at 60° C., then cooled to room temperature. A precipitate formed that was collected by filtration and washed with water (2×50 mL). The resulting solid was dried in an oven under reduced pressure to afford the product (30 g, 75%) as yellow solid. LCMS (ES, m/z): 275[M+H]+

### Step 2: 6-bromo-4-fluoro-7-methylindoline-2,3-dione

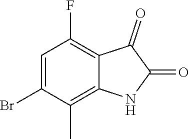

To a solution of (2E)-N-(3-bromo-5-fluoro-2-methylphenyl)-2-(N-hydroxyimino)acetamide (30 g, 109 mmol) was added to sulfuric acid (231 mL) in portions at 60° C. The resulting mixture was stirred for 1 h at 60° C., then cooled to room temperature and slowly added to ice water. A precipitate formed that was collected by filtration and washed with water (2×20 mL). The resulting solid was dried under vacuum. The residue was purified by combi-flash (EtOAc/PE=0:100-50:50) to give the product (13.8 g, 50%) as yellow solid. LCMS (ES, m/z): 258[M+H]⁺

### Step 3: 6-bromo-4-fluoro-1,7-dimethylindoline-2,3-dione

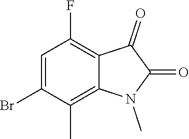

To a solution of 6-bromo-4-fluoro-7-methylindoline-2,3-dione (13.8 g, 53 mmol) in DMF (150 mL) was added sodium hydride (60%)(2.57 g, 63.6 mmol) at 0° C. for 5 min, Then added CH₃I (8.27 g, 58.3 mmol). The resulting solution was stirred for 2 h at rt. The reaction was extracted with EA. The organic layers were washed with brine. dried over anhydrous Na₂SO₄ The reaction was concentrated under reduced pressure. The residue was purified by combi-flash (EtOAc/PE=0:100-50:50) to give the product (11 g, 75%) as white solid. LCMS (ES, m/z): 272[M+H]⁺

### Step 4: 4-bromo-6-fluoro-3-methyl-2-(methylamino)benzoic acid

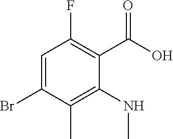

To a solution of 6-bromo-4-fluoro-1,7-dimethylindoline-2,3-dione (11 g, 40.7 mmol) and NaOH (2 M) (54 mL, 382 mmol) was added H₂O₂ (9.1 mL, 211.8 mmol) dropwise over 15 min at room temperature. The resulting mixture was stirred for an additional 3 h at room temperature, then quenched with saturated sodium sulfite (aq.) at room temperature and neutralized to pH 7 with HCl (2 M). The resulting mixture was filtered, and the filter cake washed with water (2×20 mL). The filtrate was concentrated under reduced pressure to give a residue. The residue was acidified to pH 4 with HCl (2 M). The precipitated solids were collected by filtration and washed with water (2×20 mL). The resulting solid was dried under infrared light to afford the product (8 g, 75%) as yellow solid. LCMS (ES, m/z): 262[M+H]⁺.

### Step 5: 6-bromo-4-fluoro-1,7-dimethyl-1,3-dihydro-2H-benzo[d]imidazol-2-one

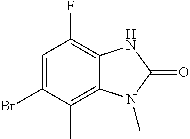

To a solution of 5-bromo-3,6-difluoro-N1-methylbenzene-1,2-diamine (8 g, 30 mmol) in the t-BuOH (80 mL) were added DIEA (10.8 g, 90 mmol) and DPPA (12.3 g, 45 mmol). The mixture was stirred at 95° C. for 12 hrs under N₂. On completion, the mixture was concentrated in vacuo. The residue was diluted with toluene (50 mL), filtered and the filtered cake was collected and dried in vacuo to give the product (4 g, 51%) as gray solid. LCMS (ES, m/z): 259[M+H]⁺.

### Step 6: 3-(5-bromo-7-fluoro-3,4-dimethyl-2-oxo-2,3-dihydro-1H-benzo[d]imidazol-1-yl)piperidine-2,6-dione

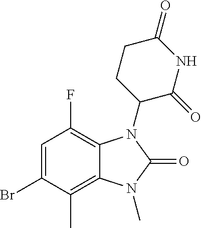 [SMILES: Cc1c(Br)cc(F)c2c1n(C)c(=O)n2C1CCC(=O)NC1=O]

To a solution of 6-bromo-4-fluoro-1,7-dimethyl-1,3-dihydro-2H-benzo[d]imidazol-2-one (4 g, 15.5 mmol) in THF (80 mL) were added sodium hydride (60%)(1.86 g, 46.5 mmol) and 3-bromopiperidine-2,6-dione (2.79 g, 30.4 mmol). The resulting solution was stirred for 12 h at 60° C. The resulting mixture was concentrated under vacuum. The residue was purified by PREP-ACHIRAL-SFC (Column: GreenSep Naphthyl 3*25 cm, 5 m; Mobile Phase A: CO₂, Mobile Phase B: MEOH:DMSO=9:1; Flow rate: 75 mL/min; Gradient (B %): isocratic 18% B; Column Temperature (° C.): 35; Back Pressure (bar): 100; Wave Length: 254 nm; RT1 (min): 4.32; RT2 (min): 6.48; Sample Solvent: ACN; Injection Volume: 3.75 mL; Number Of Runs: 8.0) to give the product (800 mg, 14%) as white solid. LCMS (ES, m/z): 370[M+H]⁺.

### Step 7: 3-(5-(4-(dimethoxymethyl)piperidin-1-yl)-7-fluoro-3,4-dimethyl-2-oxo-2,3-dihydro-1H-benzo[d]imidazol-1-yl)piperidine-2,6-dione

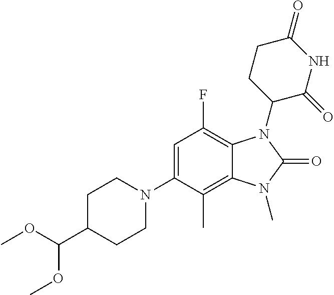 [SMILES: COC(OC)C1CCN(c2cc(F)c3c(c2C)n(C)c(=O)n3C2CCC(=O)NC2=O)CC1]

To a solution of 3-(5-bromo-7-fluoro-3,4-dimethyl-2-oxo-2,3-dihydro-1H-benzo[d]imidazol-1-yl)piperidine-2,6-dione (PH-BGUS-H-A659-5) (300 mg, 0.81 mmol) in DMF (20 mL) were added 4-(dimethoxymethyl)piperidine (191 mg, 1.2 mmol), Cs₂CO₃ (400 g, 1.2 mmol) and Pd-PEPPSI2Me-IPENT Cl (35 mg, 0.04 mmol) for 12 h at 100° C. The mixture was extracted with ethyl acetate (3×30 mL). The organic layers were washed with brine (60 mL), dried over anhydrous Na₂SO₄, filtered and concentrated under reduced pressure to give a residue. The crude was purified by combi-flash (SiO₂, PE/EA=90:10-0:100) to give the product (172.9 mg, 40%) as yellow green solid. LCMS (M+H) m/z: 449.15.

### Step 8: 1-(1-(2,6-dioxopiperidin-3-yl)-7-fluoro-3,4-dimethyl-2-oxo-2,3-dihydro-1H-benzo[d]imidazol-5-yl)piperidine-4-carbaldehyde

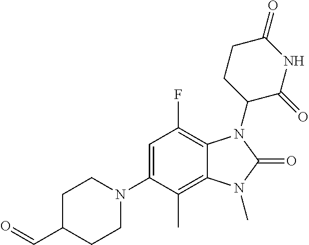 [SMILES: Cc1c(N2CCC(C=O)CC2)cc(F)c2c1n(C)c(=O)n2C1CCC(=O)NC1=O]

To a flask was added 3-(5-(4-(dimethoxymethyl)piperidin-1-yl)-7-fluoro-3,4-dimethyl-2-oxo-2,3-dihydro-1H-benzo[d]imidazol-1-yl)piperidine-2,6-dione (100 mg, 0.22 mmol) and Formic acid (5 mL). The reaction mixture was stirred for 1 hours at room temperature. The resulting mixture was concentrated to afford the crude product. LC-MS (ESI): m/z [M+H]⁺=403.6

### Step 9: (1S,3R)-3-(3-((2-((S)-1-((1-(1-((R)-2,6-dioxopiperidin-3-yl)-7-fluoro-3,4-dimethyl-2-oxo-2,3-dihydro-1H-benzo[d]imidazol-5-yl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropyl carbamate

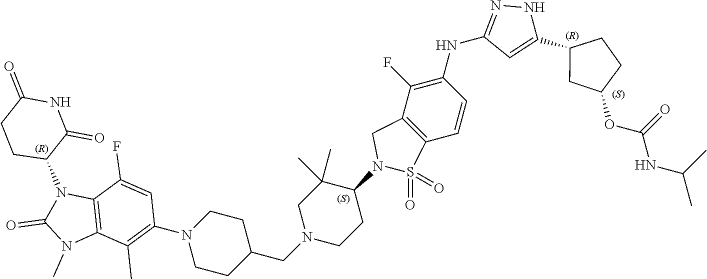

The titled compound was synthesized in the procedures similar to Example 41 and separated by chiral HPLC to give Example 237 (100% ee). Enantiomer 2 (99.8% ee); Retention time: 11.725 min. ¹H NMR (500 MHz, DMSO) δ 11.99 (s, 1H), 11.07 (s, 1H), 8.76 (s, 1H), 8.31 (t, J=7.7 Hz, 1H), 7.54 (d, J=8.7 Hz, 1H), 6.94 (d, J=7.4 Hz, 1H), 6.67 (d, J=13.2 Hz, 1H), 5.83 (s, 1H), 5.47 (d, J=7.8 Hz, 1H), 5.00 (s, 1H), 4.54 (s, 2H), 3.66-3.52 (m, 4H), 3.23 (dd, J=12.1, 3.7 Hz, 1H), 3.11-2.90 (m, 5H), 2.65-2.55 (m, 5H), 2.22-1.71 (m, 12H), 1.60 (s, 2H), 1.51 (d, J=9.8 Hz, 1H), 1.33-1.22 (m, 2H), 1.12 (s, 3H), 1.07-0.98 (m, 6H), 0.94 (s, 3H). LC-MS (ESI): m/z [M+H]⁺=935.59.

Chiral analytical method: Column: YMC Amylose-SA 4.6*250 mm 5 m Mobile phase: A for MtBE (0.1%2M NH3 MeOH) (%) and B for MeOH:DCM=50:50 (v/v); Gradient: Mobile Phase A: Mobile Phase B=20:80 (v/v); HPLC Equipment: Agilent 1260 Infinity II (ARD_LC-808); Column temperature: 25° C.

Chiral Prep-HPLC Condition: YMC Amylose-SA 4.6*250 mm 5 m Mobile phase: A for MtBE (0.1%2M NH3 MeOH)(%) and B for MeOH:DCM=50:50; Gradient: Mobile Phase A: Mobile Phase B=20:80 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 238: (1S,3R)-3-(3-((2-((4S)-1-((1-(1-(2,6-dioxopiperidin-3-yl)-4-methoxy-3-methyl-2-oxo-2,3-dihydro-1H-benzo[d]imidazol-5-yl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropylcarbamate

### Step 1: Synthesis of 4-bromo-3-methoxy-2-nitroaniline

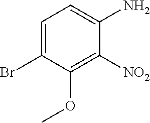

To a solution of 4-bromo-3-fluoro-2-nitroaniline (10 g, 42.7 mmol) in MeOH (500 mL) was added NaOMe (30%) (138 g, 769 mmol) at rt for overnight. The resulting mixture was concentrated under vacuum and extracted with DCM. The organic layers were washed with brine and concentrated under vacuum. The residue was purified by combi-flash (DCM/EA=100:0 to 87:13) to give the product (10 g, 95.2%) as yellow solid.

### Step 2: 5-bromo-4-methoxy-1,3-dihydro-2H-benzo[d]imidazol-2-one

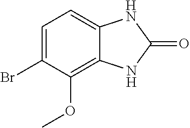

To a solution of 4-bromo-3-methoxy-2-nitroaniline (11 g, 44.7 mmol) in DMF (200 mL) were added B₂(OH)₄ (12 g, 134.14 mmol) and 4,4-Bipyridine (349 mg, 2.23 mmol) at 0° C. for 15 min under N₂. The CDI (14.48 g, 89.4 mmol) was added in it at rt for 1 h. The resulting solution was quenched with NH₄Cl solution and extracted with EA. The organic layers were washed brine and concentrated under vacuum. The filtrate was concentrated under vacuum. The residue was purified by combi-flash (DCM/MeOH=100:0 to 80:20) to give the product (5.2 g, 52.9%) as brown solid.

### Step 3: 1-(2,6-bis(benzyloxy)pyridin-3-yl)-5-bromo-4-methoxy-1,3-dihydro-2H-benzo[d]imidazol-2-one

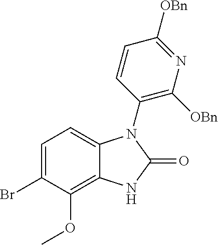 [SMILES: COc1c(Br)ccc2c1[nH]c(=O)n2-c1ccc(OCc2ccccc2)nc1OCc1ccccc1]

To a solution of 5-bromo-4-methoxy-1,3-dihydro-2H-benzo[d]imidazol-2-one (5.2 g, 21.48 mmol) in dioxane (100 mL) were added 2,6-bis(benzyloxy)-3-(4,4,5,5-tetramethyl-1,3,2-dioxaborolan-2-yl)pyridine (10.8 g, 33.23 mmol), Py (5.09 g, 64.44 mmol), Cu(OAc)₂ (4.27 g, 21.48 mmol) and 4AMS (2.5 g) at 80° C. for overnight under 02. The reaction was concentrated under vacuum. The residue was purified by combi-flash (PE/EA=100:0 to 67:37) to give the product (2 g, 10.5%) as light brown solid.

### Step 4: 1-(2,6-bis(benzyloxy)pyridin-3-yl)-5-bromo-4-methoxy-3-methyl-1,3-dihydro-2H-benzo[d]imidazol-2-one

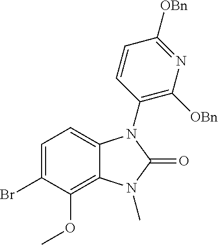 [SMILES: COc1c(Br)ccc2c1n(C)c(=O)n2-c1ccc(OCc2ccccc2)nc1OCc1ccccc1]

To a solution of 1-(2,6-bis(benzyloxy)pyridin-3-yl)-5-bromo-4-methoxy-1,3-dihydro-2H-benzo[d]imidazol-2-one (2.1 g, 3.95 mmol) in DMF (60 mL) were added K₂CO₃ (1.09 g, 7.91 mmol) and CH₃I (1.12 g, 7.91 mmol) at rt for 2 h. The resulting mixture was quenched with water and extracted with EA. The organic layers were washed with brine. The organic layers were concentrated under vacuum. The residue was purified by combi-flash (PE/EA=100:0 to 68:32) to give the product (0.95 g, 44.1%) as brown solid.

### Step 5: 1-(2,6-bis(benzyloxy)pyridin-3-yl)-5-(4-(dimethoxymethyl)piperidin-1-yl)-4-methoxy-3-methyl-1,3-dihydro-2H-benzo[d]imidazol-2-one

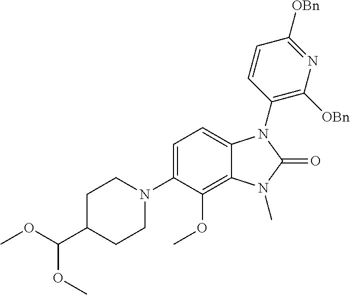 [SMILES: COc1c(N2CCC(C(OC)OC)CC2)ccc2c1n(C)c(=O)n2-c1ccc(OCc2ccccc2)nc1OCc1ccccc1]

To a solution of 1-(2,6-bis(benzyloxy)pyridin-3-yl)-5-bromo-4-methoxy-3-methyl-1,3-dihydro-2H-benzo[d]imidazol-2-one (PH-BGUS-H-A638-5) (1.2 g, 2.2 mmol) in dry-dioxane (20 mL) were added 4-(dimethoxymethyl)piperidine (525 mg, 3.3 mmol), Pd-PEPPSI-IPentCl 2-methylpyridine (92.4 mg, 0.11 mmol) and Cs₂CO₃ (1.07 g, 3.3 mmol) at 100° C. for 2 h under N₂ atmosphere. The resulting mixture was concentrated under vacuum. The residue was purified by combi-flash (PE/EA=100:0 to 74:26) to give the product (0.8 g, 58.4%) as light yellow solid.

### Step 6: 3-(5-(4-(dimethoxymethyl)piperidin-1-yl)-4-methoxy-3-methyl-2-oxo-2,3-dihydro-1H-benzo[d]imidazol-1-yl)piperidine-2,6-dione

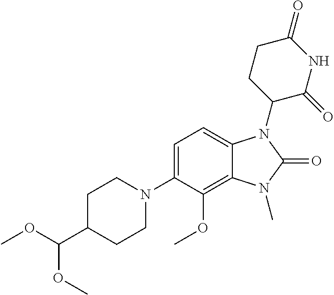 [SMILES: COc1c(N2CCC(C(OC)OC)CC2)ccc2c1n(C)c(=O)n2C1CCC(=O)NC1=O]

To a solution of 1-(2,6-bis(benzyloxy)pyridin-3-yl)-5-(4-(dimethoxymethyl)piperidin-1-yl)-4-methoxy-3-methyl-1,3-dihydro-2H-benzo[d]imidazol-2-one (0.77 g, 1.23 mmol) in dry-THF (100 mL) was added Pd/C (0.77 g). The mixture was hydrogenated at room temperature for overnight under hydrogen atmosphere using a hydrogen balloon. The resulting mixture was filtered the filter cake was washed with THF (100 mL). The filtrate was concentrated under vacuum. The residue was without purified to give the product (0.414 g, 75.4%) as light grey solid. LCMS (ESI) m/e [M+1]⁺=447.

### Step 7: 1-(1-(2,6-dioxopiperidin-3-yl)-4-methoxy-3-methyl-2-oxo-2,3-dihydro-1H-benzo[d]imidazol-5-yl)piperidine-4-carbaldehyde

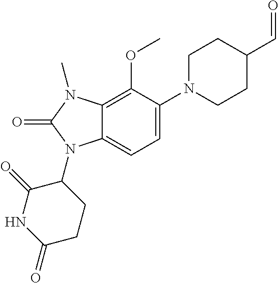 [SMILES: COc1c(N2CCC(C=O)CC2)ccc2c1n(C)c(=O)n2C1CCC(=O)NC1=O]

3-(5-(4-(dimethoxymethyl)piperidin-1-yl)-4-methoxy-3-methyl-2-oxo-2,3-dihydro-1H-benzo[d]imidazol-1-yl)piperidine-2,6-dione (50.0 mg, 0.11 mmol) was dissolved in formic acid (3 mL), then the mixture was stirred for overnight at rt. The reaction was detected by LCMS, after completion the solution was concentrated under reduced pressure and used for the next step directly. (44.0 mg, quantitative yield). LC-MS (ESI): m/z [M+H]⁺=401.4.

### Step 2: (1S,3R)-3-(3-((2-((4S)-1-((1-(1-(2,6-dioxopiperidin-3-yl)-4-methoxy-3-methyl-2-oxo-2,3-dihydro-1H-benzo[d]imidazol-5-yl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropylcarbamate

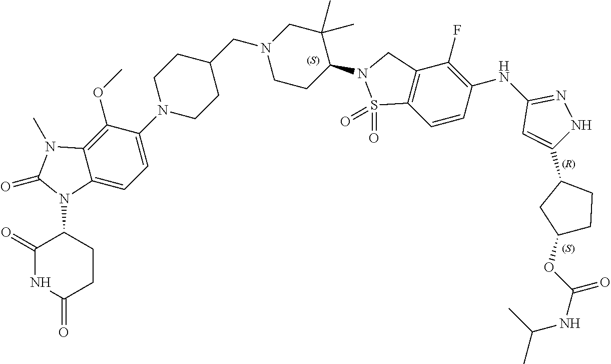

To a solution of 1-(1-(2,6-dioxopiperidin-3-yl)-4-methoxy-3-methyl-2-oxo-2,3-dihydro-1H-benzo[d]imidazol-5-yl)piperidine-4-carbaldehyde (44.0 mg, 0.11 mmol) in DMA (3 mL), (1S,3R)-3-(3-((2-((S)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropylcarbamate (65.76 mg, 0.12 mmol) were added at rt. The resulting mixture was stirred for 10 mins. Then sodium triacetoxyborohydride (70.0 mg, 0.33 mmol) was added into the mixture and stirred for 2 h. The reaction was detected by LCMS, after the residue was purified by silica gel chromatography, eluting with DCM/MeOH (10:1; R_{f}=0.32) to afford the crude product, then the crude product was purified by was purified by Prep-HPLC (Column: Sunfire C18, 19*150 mm, 5 μm; Mobile Phase A: H2O (0.1% TFA), Mobile Phase B: ACN; Flow Rate: 17 mL/min; Room Temperature; Detector: UV 214 & 254 nm; Gradient: 23% to 48% B in 11 min, 48% to 90% B in 0.2 min, 90% B hold 2 min, 90% to 23% B in 0.3 min, 23% B hold 1.5 min. to give the product (54.49 mg, 53% yield). ¹H NMR (500 MHz, DMSO-d₆) δ 11.98 (brs, 1H), 11.03 (s, 1H), 8.78 (s, 1H), 8.66-8.60 (m, 1H), 8.38-8.29 (m, 1H), 7.58 (d, J=8.5 Hz, 1H), 6.94 (d, J=7.7 Hz, 1H), 6.79 (d, J=8.5 Hz, 1H), 6.69 (d, J=8.5 Hz, 1H), 5.85 (s, 1H), 5.33-5.30 (m, 1H), 5.10-4.89 (m, 1H), 4.59 (s, 2H), 3.95-3.90 (m, 1H), 3.45-3.34 (m, 6H), 3.33-3.27 (m, 3H), 3.23-3.14 (m, 1H), 3.12-3.01 (m, 13.5 Hz, 4H), 2.79-2.68 (m, 1H), 2.61-2.50 (m, 3H), 2.38-2.33 (m, 2H), 2.10-1.98 (m, 3H), 1.95-1.91 (m, 1H), 1.90-1.79 (m, 3H), 1.72-1.66 (m, 2H), 1.62-1.59 (m, 1H), 1.53-1.45 (m, 2H), 1.10-1.03 (m, 3H), 1.03-1.01 (m, 9H). LC-MS (ESI): m/z [M+H]⁺=933.56.

### Example 239: (1S,3R)-3-(3-((2-((4S)-1-((1-(1-(2,6-dioxopiperidin-3-yl)-4-fluoro-3-methyl-2-oxo-2,3-dihydro-1H-benzo[d]imidazol-5-yl)piperidin-4-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl isopropylcarbamate

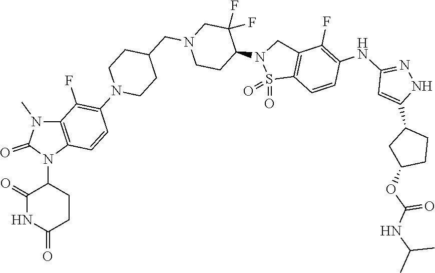 [SMILES: CC(C)NC(=O)O[C@H]1CC[C@@H](c2cc(Nc3ccc4c(c3F)CN([C@H]3CCN(CC5CCN(c6ccc7c(c6F)n(C)c(=O)n7C6CCC(=O)NC6=O)CC5)CC3(F)F)S4(=O)=O)n[nH]2)C1]

The titled compound was synthesized in the procedures similar to Example 37. ¹H NMR (500 MHz, DMSO) δ 12.00 (s, 1H), 11.10 (s, 1H), 8.82 (s, 1H), 8.33 (t, J=8.0 Hz, 1H), 7.58 (d, J=8.7 Hz, 1H), 6.94 (d, J=7.6 Hz, 1H), 5.84 (s, 1H), 5.33 (dd, J=12.8, 5.4 Hz, 1H), 5.06-4.96 (m, 1H), 4.75 (d, J=14.6 Hz, 1H), 4.54 (d, J=12.9 Hz, 1H), 4.09-3.86 (m, 1H), 3.64-3.52 (m, 1H), 3.47 (s, 3H), 3.26-3.13 (m, 4H), 3.10-3.02 (m, 1H), 3.00-2.84 (m, 2H), 2.72-2.64 (m, 3H), 2.61-2.55 (m, 2H), 2.34-2.28 (m, 3H), 2.26-2.14 (m, 2H), 2.06-1.98 (m, 2H), 1.96-1.86 (m, 1H), 1.84-1.77 (m, 3H), 1.76-1.58 (m, 4H), 1.36-1.24 (m, 2H), 1.03 (d, J=6.3 Hz, 6H). LC-MS (ESI): m/z [M+H]⁺=929.4;

### Example 240a and 240b: (1S,3R)-3-(3-((2-((4R)-1′-(1-(2,6-dioxopiperidin-3-yl)-4-fluoro-3-methyl-2-oxo-2,3-dihydro-1H-benzo[d]imidazol-5-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-((4S)-1′-(1-(2,6-dioxopiperidin-3-yl)-4-fluoro-3-methyl-2-oxo-2,3-dihydro-1H-benzo[d]imidazol-5-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

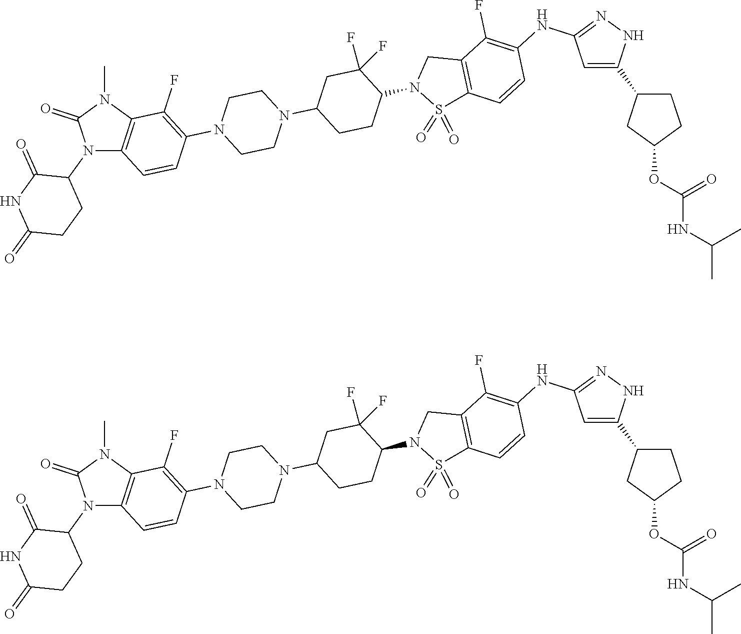

The titled compound was synthesized in the procedures similar to Example 45 in a racemic form, which was further separated by chiral HPLC to give.

Enantiomer 1 (Example 240a, 100% ee); Retention time: 5.884 min. ¹H NMR (500 MHz, DMSO) δ 12.01 (s, 1H), 11.10 (s, 1H), 8.82 (s, 1H), 8.32 (d, J=7.6 Hz, 1H), 7.58 (d, J=8.7 Hz, 1H), 6.94 (d, J=7.1 Hz, 1H), 6.85 (d, J=8.3 Hz, 1H), 6.74 (t, J=8.1 Hz, 1H), 5.84 (s, 1H), 5.33 (dd, J=12.4, 5.1 Hz, 1H), 5.06-4.96 (m, 1H), 4.75 (d, J=14.3 Hz, 1H), 4.54 (d, J=13.7 Hz, 1H), 4.05-3.92 (m, 1H), 3.63-3.54 (m, 1H), 3.47 (s, 3H), 3.14-2.98 (m, 2H), 2.96-2.61 (m, 11H), 2.46-2.42 (m, 1H), 2.22-2.10 (m, 1H), 2.08-1.96 (m, 2H), 1.95-1.80 (m, 4H), 1.77-1.55 (m, 5H), 1.03 (d, J=6.1 Hz, 6H). LC-MS (ESI): m/z [M+H]⁺=915.4;

Enantiomer 2 (Example 240b, 100% ee); Retention time: 12.796, 14.81 min. ¹H NMR (500 MHz, DMSO) δ 12.01 (s, 1H), 11.10 (s, 1H), 8.82 (s, 1H), 8.32 (d, J=7.6 Hz, 1H), 7.58 (d, J=8.7 Hz, 1H), 6.94 (d, J=7.1 Hz, 1H), 6.85 (d, J=8.3 Hz, 1H), 6.74 (t, J=8.1 Hz, 1H), 5.84 (s, 1H), 5.33 (dd, J=12.4, 5.1 Hz, 1H), 5.06-4.96 (m, 1H), 4.75 (d, J=14.3 Hz, 1H), 4.54 (d, J=13.7 Hz, 1H), 4.05-3.92 (m, 1H), 3.63-3.54 (m, 1H), 3.47 (s, 3H), 3.14-2.98 (m, 2H), 2.96-2.61 (m, 11H), 2.46-2.42 (m, 1H), 2.22-2.10 (m, 1H), 2.08-1.96 (m, 2H), 1.95-1.80 (m, 4H), 1.77-1.55 (m, 5H), 1.03 (d, J=6.1 Hz, 6H). LC-MS (ESI): m/z [M+H]⁺=915.4;

Chiral analytical method: Column: ICHIRALPAK SA 4.6*150 mm 5 m; Mobile phase: A for MtBE (0.1%2M NH3 MeOH) (%) and B for MeOH:DCM=50:50(%); Gradient: Mobile Phase A: Mobile Phase B=30:70 (v/v); HPLC Equipment: HPLC-Agilent, Back pressure: 100 bar; Column temperature: 35° C.

Chiral Prep-HPLC Condition: CHIRALPAK SA 250*20.0 mml, 5 μm; Mobile phase: A for MtBE (0.1%2M NH3 MeOH) (%) and B for MeOH:DCM=50:50; Gradient: Mobile Phase A: Mobile Phase B=30:70 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 241a and 241b: (1S,3R)-3-(3-((2-((S)-1′-(1-((S)-2,6-dioxopiperidin-3-yl)-3-ethyl-4-fluoro-2-oxo-2,3-dihydro-1H-benzo[d]imidazol-5-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-((S)-1′-(1-((R)-2,6-dioxopiperidin-3-yl)-3-ethyl-4-fluoro-2-oxo-2,3-dihydro-1H-benzo[d]imidazol-5-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-((R)-1′-(1-((S)-2,6-dioxopiperidin-3-yl)-3-ethyl-4-fluoro-2-oxo-2,3-dihydro-1H-benzo[d]imidazol-5-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-((R)-1′-(1-((R)-2,6-dioxopiperidin-3-yl)-3-ethyl-4-fluoro-2-oxo-2,3-dihydro-1H-benzo[d]imidazol-5-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

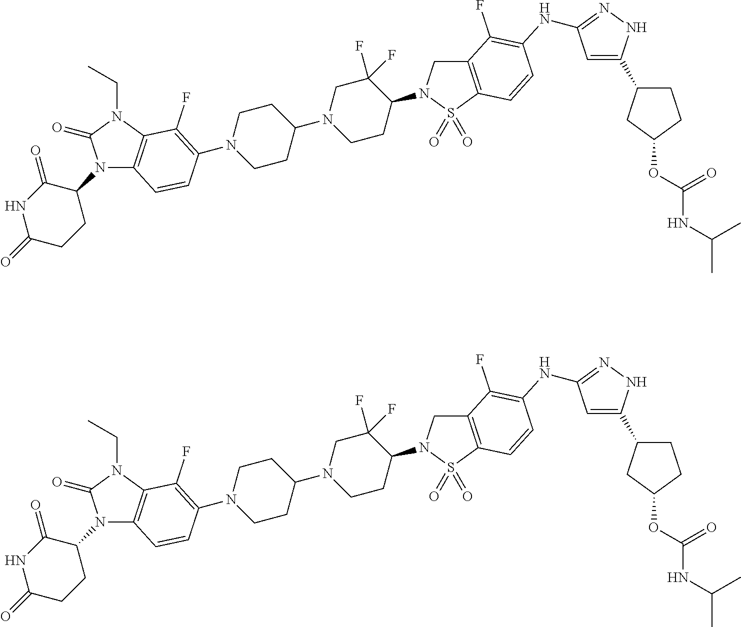 [SMILES: CCn1c(=O)n([C@H]2CCC(=O)NC2=O)c2ccc(N3CCC(N4CC[C@H](N5Cc6c(ccc(Nc7cc([C@@H]8CC[C@H](OC(=O)NC(C)C)C8)[nH]n7)c6F)S5(=O)=O)C(F)(F)C4)CC3)c(F)c21 ; CCn1c(=O)n([C@@H]2CCC(=O)NC2=O)c2ccc(N3CCC(N4CC[C@H](N5Cc6c(ccc(Nc7cc([C@@H]8CC[C@H](OC(=O)NC(C)C)C8)[nH]n7)c6F)S5(=O)=O)C(F)(F)C4)CC3)c(F)c21]

### Step 1: 3-(3-ethyl-4-fluoro-2-oxo-5-(4-oxopiperidin-1-yl)-2,3-dihydro-1H-benzo[d]imidazol-1-yl)piperidine-2,6-dione

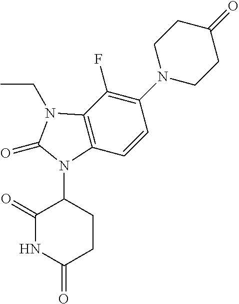 [SMILES: CCn1c(=O)n(C2CCC(=O)NC2=O)c2ccc(N3CCC(=O)CC3)c(F)c21]

The titled compound was synthesized in the procedures similar to Example 45. LC-MS (ESI): m/z [M+H]⁺=389.4

### Step 2: (1S,3R)-3-(3-((2-((S)-1′-(1-((S)-2,6-dioxopiperidin-3-yl)-3-ethyl-4-fluoro-2-oxo-2,3-dihydro-1H-benzo[d]imidazol-5-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-((S)-1′-(1-((R)-2,6-dioxopiperidin-3-yl)-3-ethyl-4-fluoro-2-oxo-2,3-dihydro-1H-benzo[d]imidazol-5-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-((R)-1′-(1-((S)-2,6-dioxopiperidin-3-yl)-3-ethyl-4-fluoro-2-oxo-2,3-dihydro-1H-benzo[d]imidazol-5-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-((R)-1′-(1-((R)-2,6-dioxopiperidin-3-yl)-3-ethyl-4-fluoro-2-oxo-2,3-dihydro-1H-benzo[d]imidazol-5-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

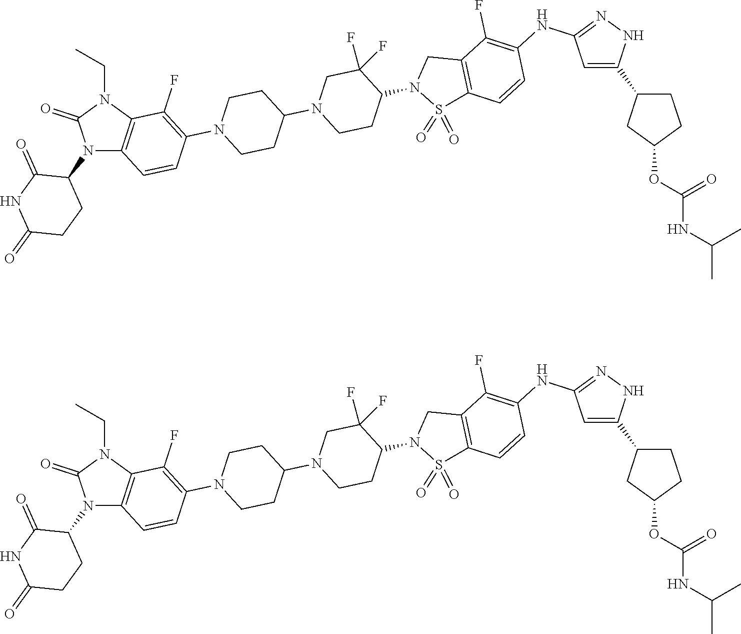

The titled compound was synthesized in the procedures similar to Example 240 in a racemate form, which was further separated by chiral HPLC to give:

Enantiomer 1 (Example 241a, 100% ee); Retention time: 4.505 min. ¹H NMR (500 MHz, DMSO) δ 12.01 (s, 1H), 11.10 (s, 1H), 8.82 (s, 1H), 8.33 (t, J=8.0 Hz, 1H), 7.58 (d, J=8.6 Hz, 1H), 6.94 (s, 1H), 6.86 (d, J=8.6 Hz, 1H), 6.76 (d, J=8.0 Hz, 1H), 5.84 (s, 1H), 5.39-5.28 (m, 1H), 5.06-4.96 (m, 1H), 4.75 (d, J=14.4 Hz, 1H), 4.54 (d, J=13.2 Hz, 1H), 4.02-3.86 (m, 3H), 3.65-3.53-3.54 (m, 1H), 3.26-3.18 (m, 1H), 3.13-3.00 (m, 2H), 2.92-2.68 (m, 10H), 2.20-2.10 (m, 1H), 2.07-1.96 (m, 3H), 1.96-1.88 (m, 1H), 1.88-1.80 (m, 3H), 1.78-1.56 (d, J=44.7 Hz, 5H), 1.28-1.20 (m, 6H), 1.03 (d, J=5.6 Hz, 6H). LC-MS (ESI): m/z [M+H]⁺=929.4;

Enantiomer 2 (Example 241b, 100% ee); Retention time: 4.293 min. ¹H NMR (500 MHz, DMSO) δ 12.01 (s, 1H), 11.10 (s, 1H), 8.82 (s, 1H), 8.33 (t, J=8.0 Hz, 1H), 7.58 (d, J=8.6 Hz, 1H), 6.94 (s, 1H), 6.86 (d, J=8.6 Hz, 1H), 6.76 (d, J=8.0 Hz, 1H), 5.84 (s, 1H), 5.39-5.28 (m, 1H), 5.06-4.96 (m, 1H), 4.75 (d, J=14.4 Hz, 1H), 4.54 (d, J=13.2 Hz, 1H), 4.02-3.86 (m, 3H), 3.65-3.53-3.54 (m, 1H), 3.26-3.18 (m, 1H), 3.13-3.00 (m, 2H), 2.92-2.68 (m, 10H), 2.20-2.10 (m, 1H), 2.07-1.96 (m, 3H), 1.96-1.88 (m, 1H), 1.88-1.80 (m, 3H), 1.78-1.56 (d, J=44.7 Hz, 5H), 1.28-1.20 (m, 6H), 1.03 (d, J=5.6 Hz, 6H). LC-MS (ESI): m/z [M+H]⁺=929.4;

Enantiomer 1 (Example 1c, 100% ee); Retention time: 7.9 min. ¹H NMR (500 MHz, DMSO) δ 12.01 (s, 1H), 11.10 (s, 1H), 8.82 (s, 1H), 8.33 (t, J=8.0 Hz, 1H), 7.58 (d, J=8.6 Hz, 1H), 6.94 (s, 1H), 6.86 (d, J=8.6 Hz, 1H), 6.76 (d, J=8.0 Hz, 1H), 5.84 (s, 1H), 5.39-5.28 (m, 1H), 5.06-4.96 (m, 1H), 4.75 (d, J=14.4 Hz, 1H), 4.54 (d, J=13.2 Hz, 1H), 4.02-3.86 (m, 3H), 3.65-3.53-3.54 (m, 1H), 3.26-3.18 (m, 1H), 3.13-3.00 (m, 2H), 2.92-2.68 (m, 10H), 2.20-2.10 (m, 1H), 2.07-1.96 (m, 3H), 1.96-1.88 (m, 1H), 1.88-1.80 (m, 3H), 1.78-1.56 (d, J=44.7 Hz, 5H), 1.28-1.20 (m, 6H), 1.03 (d, J=5.6 Hz, 6H). LC-MS (ESI): m/z [M+H]⁺=929.4;

Enantiomer 1 (Example Id, 100% ee); Retention time: 7.23 min. ¹H NMR (500 MHz, DMSO) δ 12.01 (s, 1H), 11.10 (s, 1H), 8.82 (s, 1H), 8.33 (t, J=8.0 Hz, 1H), 7.58 (d, J=8.6 Hz, 1H), 6.94 (s, 1H), 6.86 (d, J=8.6 Hz, 1H), 6.76 (d, J=8.0 Hz, 1H), 5.84 (s, 1H), 5.39-5.28 (m, 1H), 5.06-4.96 (m, 1H), 4.75 (d, J=14.4 Hz, 1H), 4.54 (d, J=13.2 Hz, 1H), 4.02-3.86 (m, 3H), 3.65-3.53-3.54 (m, 1H), 3.26-3.18 (m, 1H), 3.13-3.00 (m, 2H), 2.92-2.68 (m, 10H), 2.20-2.10 (m, 1H), 2.07-1.96 (m, 3H), 1.96-1.88 (m, 1H), 1.88-1.80 (m, 3H), 1.78-1.56 (d, J=44.7 Hz, 5H), 1.28-1.20 (m, 6H), 1.03 (d, J=5.6 Hz, 6H). LC-MS (ESI): m/z [M+H]⁺=929.4;

Chiral analytical method: Column: ICHIRALPAK SA 4.6*150 mm 5 m; Mobile phase: A for MtBE (0.1%2M NH3 MeOH) (%) and B for MeOH:DCM=50:50(%); Gradient: Mobile Phase A: Mobile Phase B=30:70 (v/v); HPLC Equipment: HPLC-Agilent, Back pressure: 100 bar; Column temperature: 35° C.

Chiral Prep-HPLC Condition: CHIRALPAK SA 250*20.0 mml, 5 μm; Mobile phase: A for MtBE (0.1%2M NH3 MeOH) (%) and B for MeOH:DCM=50:50; Gradient: Mobile Phase A: Mobile Phase B=30:70 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 242a and 242b: (1S,3R)-3-(3-((2-((4S)-1′-(1-(2,6-dioxopiperidin-3-yl)-4-fluoro-3-isopropyl-2-oxo-2,3-dihydro-1H-benzo[d]imidazol-5-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-((4R)-1′-(1-(2,6-dioxopiperidin-3-yl)-4-fluoro-3-isopropyl-2-oxo-2,3-dihydro-1H-benzo[d]imidazol-5-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

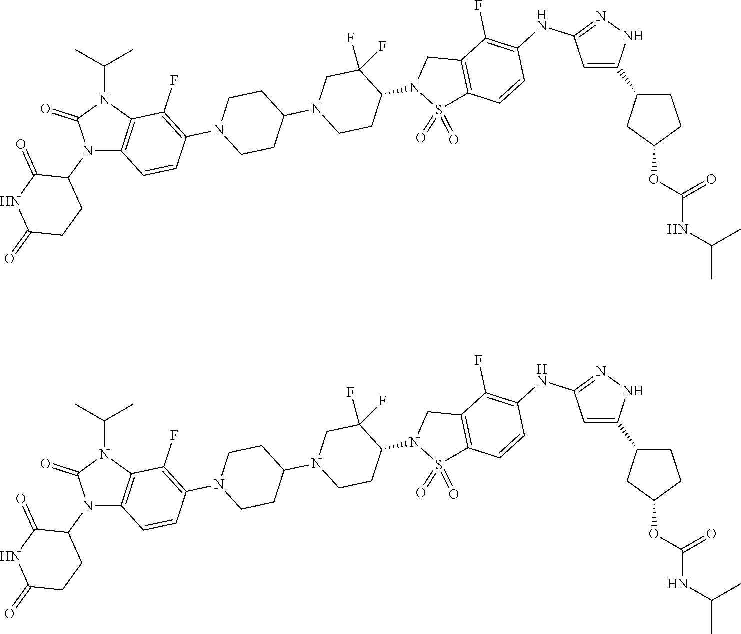 [SMILES: CC(C)NC(=O)O[C@H]1CC[C@@H](c2cc(Nc3ccc4c(c3F)CN([C@@H]3CCN(C5CCN(c6ccc7c(c6F)n(C(C)C)c(=O)n7C6CCC(=O)NC6=O)CC5)CC3(F)F)S4(=O)=O)n[nH]2)C1 ; CC(C)NC(=O)O[C@H]1CC[C@@H](c2cc(Nc3ccc4c(c3F)CN([C@@H]3CCN(C5CCN(c6ccc7c(c6F)n(C(C)C)c(=O)n7C6CCC(=O)NC6=O)CC5)CC3(F)F)S4(=O)=O)n[nH]2)C1]

### Step 1: 3-(4-fluoro-3-isopropyl-2-oxo-5-(4-oxopiperidin-1-yl)-2,3-dihydro-1H-benzo[d]imidazol-1-yl)piperidine-2,6-dione

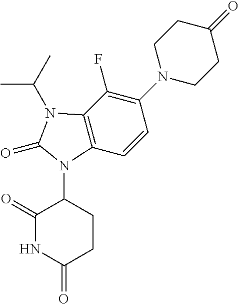 [SMILES: CC(C)n1c(=O)n(C2CCC(=O)NC2=O)c2ccc(N3CCC(=O)CC3)c(F)c21]

The titled compound was synthesized in the procedures similar to Example 45. LC-MS (ESI): m/z [M+H]⁺=4034.

### Step 2: (1S,3R)-3-(3-((2-((4S)-1′-(1-(2,6-dioxopiperidin-3-yl)-4-fluoro-3-isopropyl-2-oxo-2,3-dihydro-1H-benzo[d]imidazol-5-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-((4R)-1′-(1-(2,6-dioxopiperidin-3-yl)-4-fluoro-3-isopropyl-2-oxo-2,3-dihydro-1H-benzo[d]imidazol-5-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

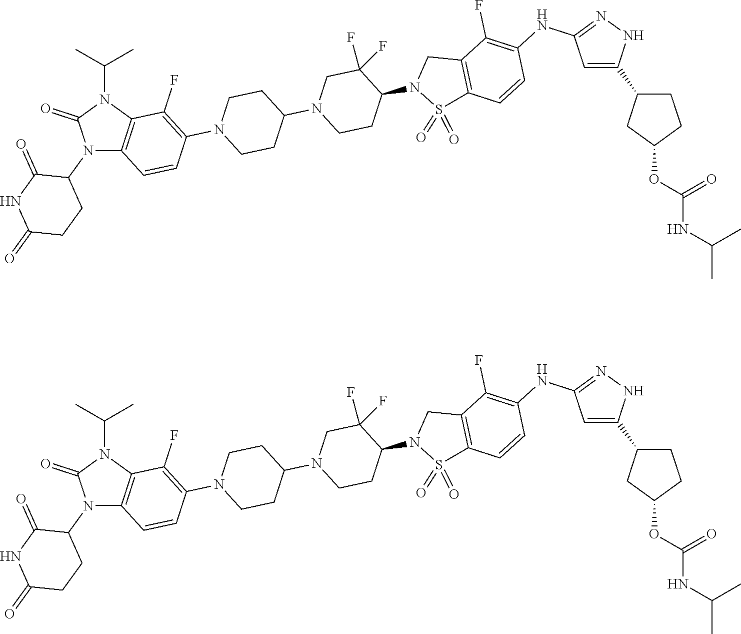 [SMILES: CC(C)NC(=O)O[C@H]1CC[C@@H](c2cc(Nc3ccc4c(c3F)CN([C@H]3CCN(C5CCN(c6ccc7c(c6F)n(C(C)C)c(=O)n7C6CCC(=O)NC6=O)CC5)CC3(F)F)S4(=O)=O)n[nH]2)C1 ; CC(C)NC(=O)O[C@H]1CC[C@@H](c2cc(Nc3ccc4c(c3F)CN([C@H]3CCN(C5CCN(c6ccc7c(c6F)n(C(C)C)c(=O)n7C6CCC(=O)NC6=O)CC5)CC3(F)F)S4(=O)=O)n[nH]2)C1]

The titled compound was synthesized in the procedures similar to Example 240 in a racemic form, which was further separated by chiral HPLC to give:

Enantiomer 1 (Example 242a, 100% ee); Retention time: 6.3 min. ¹H NMR (500 MHz, DMSO) δ 12.01 (s, 1H), 11.09 (s, 1H), 8.82 (s, 1H), 8.33 (t, J=7.7 Hz, 1H), 7.58 (d, J=8.6 Hz, 1H), 6.94 (s, 1H), 6.86 (d, J=8.4 Hz, 1H), 6.79 (t, J=8.0 Hz, 1H), 5.84 (s, 1H), 5.32 (d, J=13.2 Hz, 1H), 5.06-4.96 (m, 1H), 4.76 (dd, J=16.2, 10.5 Hz, 2H), 4.54 (d, J=12.9 Hz, 1H), 4.04-3.92 (m, 1H), 3.65-3.53-3.52 (m, 1H), 3.10-2.97 (m, 2H), 2.95-2.65 (m, 10H), 2.43-2.40 (m, 1H), 2.20-2.10 (m, 1H), 2.07-1.98 (m, 2H), 1.96-1.79 (m, 5H), 1.76-1.56 (m, 6H), 1.42 (d, J=6.8 Hz, 6H), 1.03 (d, J=6.3 Hz, 6H). LC-MS (ESI): m/z [M+H]⁺=943.4;

Enantiomer 2 (Example 242b, 100% ee); Retention time: 12.91 min. ¹H NMR (500 MHz, DMSO) δ 12.01 (s, 1H), 11.09 (s, 1H), 8.82 (s, 1H), 8.33 (t, J=7.7 Hz, 1H), 7.58 (d, J=8.6 Hz, 1H), 6.94 (s, 1H), 6.86 (d, J=8.4 Hz, 1H), 6.79 (t, J=8.0 Hz, 1H), 5.84 (s, 1H), 5.32 (d, J=13.2 Hz, 1H), 5.06-4.96 (m, 1H), 4.76 (dd, J=16.2, 10.5 Hz, 2H), 4.54 (d, J=12.9 Hz, 1H), 4.04-3.92 (m, 1H), 3.65-3.53-3.52 (m, 1H), 3.10-2.97 (m, 2H), 2.95-2.65 (m, 10H), 2.43-2.40 (m, 1H), 2.20-2.10 (m, 1H), 2.07-1.98 (m, 2H), 1.96-1.79 (m, 5H), 1.76-1.56 (m, 6H), 1.42 (d, J=6.8 Hz, 6H), 1.03 (d, J=6.3 Hz, 6H). LC-MS (ESI): m/z [M+H]⁺=943.4;

Chiral analytical method: Column: ICHIRALPAK IE 4.6*150 mm 5 m; Mobile phase: A for MtBE (0.1%2M NH3 MeOH) (%) and B for MeOH:DCM=50:50(%); Gradient: Mobile Phase A: Mobile Phase B=20:80 (v/v); HPLC Equipment: HPLC-Agilent, Back pressure: 100 bar; Column temperature: 35° C.

Chiral Prep-HPLC Condition: CHIRALPAK IE 250*20.0 mml, 5 μm; Mobile phase: A for MtBE (0.1%2M NH3 MeOH) (%) and B for MeOH:DCM=50:50; Gradient: Mobile Phase A: Mobile Phase B=20:80 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 243: (1S,3R)-3-(3-((2-((4R)-1-(1-(1-(1-(2,6-dioxopiperidin-3-yl)-4-fluoro-3-methyl-2-oxo-2,3-dihydro-1H-benzo[d]imidazol-5-yl)piperidin-4-yl)azetidin-3-yl)-3,3-difluoropiperidin-4-yl)-4-fluoro-1,1-dioxido-2,3-dihydrobenzo[d]isothiazol-5-yl)amino)-1H-pyrazol-5-yl)cyclopentyl isopropylcarbamate

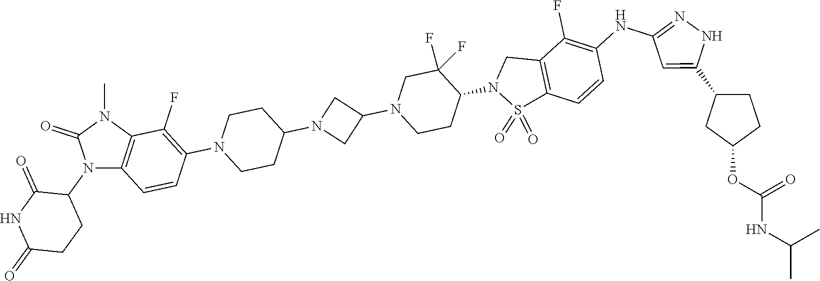 [SMILES: CC(C)NC(=O)O[C@H]1CC[C@@H](c2cc(Nc3ccc4c(c3F)CN([C@@H]3CCN(C5CN(C6CCN(c7ccc8c(c7F)n(C)c(=O)n8C7CCC(=O)NC7=O)CC6)C5)CC3(F)F)S4(=O)=O)n[nH]2)C1]

### Step 1: (1S,3R)-3-(3-((2-((R)-1-(azetidin-3-yl)-3,3-difluoropiperidin-4-yl)-4-fluoro-1,1-dioxido-2,3-dihydrobenzo[d]isothiazol-5-yl)amino)-1H-pyrazol-5-yl)cyclopentyl isopropylcarbamate

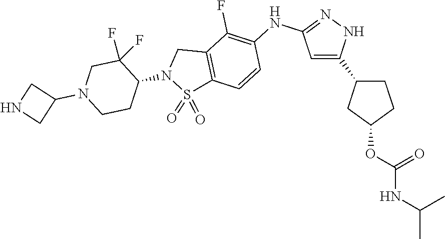 [SMILES: CC(C)NC(=O)O[C@H]1CC[C@@H](c2cc(Nc3ccc4c(c3F)CN([C@@H]3CCN(C5CNC5)CC3(F)F)S4(=O)=O)n[nH]2)C1]

The titled compound was synthesized in the procedures similar to Example 224. LC-MS (ESI): m/z [M+H]⁺=612.6.

### Step 2: (1S,3R)-3-(3-((2-((4R)-1-(1-(1-(1-(2,6-dioxopiperidin-3-yl)-4-fluoro-3-methyl-2-oxo-2,3-dihydro-1H-benzo[d]imidazol-5-yl)piperidin-4-yl)azetidin-3-yl)-3,3-difluoropiperidin-4-yl)-4-fluoro-1,1-dioxido-2,3-dihydrobenzo[d]isothiazol-5-yl)amino)-1H-pyrazol-5-yl)cyclopentyl isopropylcarbamate

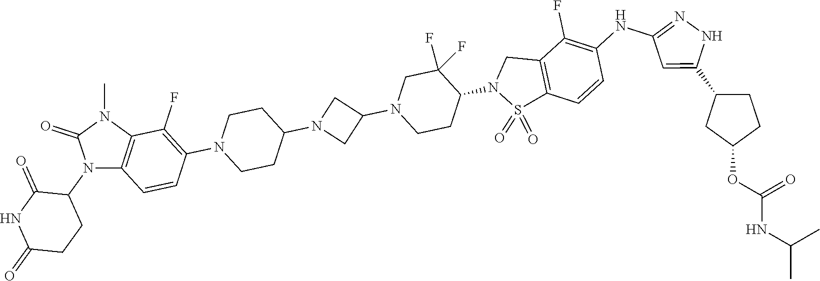 [SMILES: CC(C)NC(=O)O[C@H]1CC[C@@H](c2cc(Nc3ccc4c(c3F)CN([C@@H]3CCN(C5CN(C6CCN(c7ccc8c(c7F)n(C)c(=O)n8C7CCC(=O)NC7=O)CC6)C5)CC3(F)F)S4(=O)=O)n[nH]2)C1]

The titled compound was synthesized in the procedures similar to Example 225. ¹H NMR (500 MHz, DMSO) δ 12.03 (s, 1H), 11.11 (s, 1H), 8.82 (s, 1H), 8.31 (s, 1H), 7.61 (t, J=15.0 Hz, 1H), 6.93 (s, 1H), 6.87 (d, J=8.4 Hz, 1H), 6.76 (s, 1H), 5.84 (s, 1H), 5.34 (dd, J=12.8, 5.2 Hz, 1H), 5.06-4.96 (s, 1H), 4.75 (d, J=14.3 Hz, 1H), 4.54 (d, J=14.8 Hz, 1H), 4.30-4.20 (m, 2H), 4.16-4.04 (m, 3H), 3.62-3.56 (m, 4H), 3.35-3.30 (m, 3H), 3.12-3.02 (m, 2H), 2.98-2.84 (m, 2H), 2.76-2.66 (m, 5H), 2.44-2.41 (m, 1H), 2.24-2.12 (m, 2H), 2.08-1.96 (m, 5H), 1.94-1.82 (m, 3H), 1.80-1.66 (m, 3H), 1.64-1.44 (m, 4H), 1.03 (d, J=6.3 Hz, 6H). LC-MS (ESI): m/z [M+H]⁺=970.4;

### Example 244 and 245: (1S,3R)-3-(3-((2-((R)-1′-(1-((S)-2,6-dioxopiperidin-3-yl)-7-fluoro-3-methyl-2-oxo-2,3-dihydro-1H-benzo[d]imidazol-5-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-((R)-1′-(1-((R)-2,6-dioxopiperidin-3-yl)-7-fluoro-3-methyl-2-oxo-2,3-dihydro-1H-benzo[d]imidazol-5-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

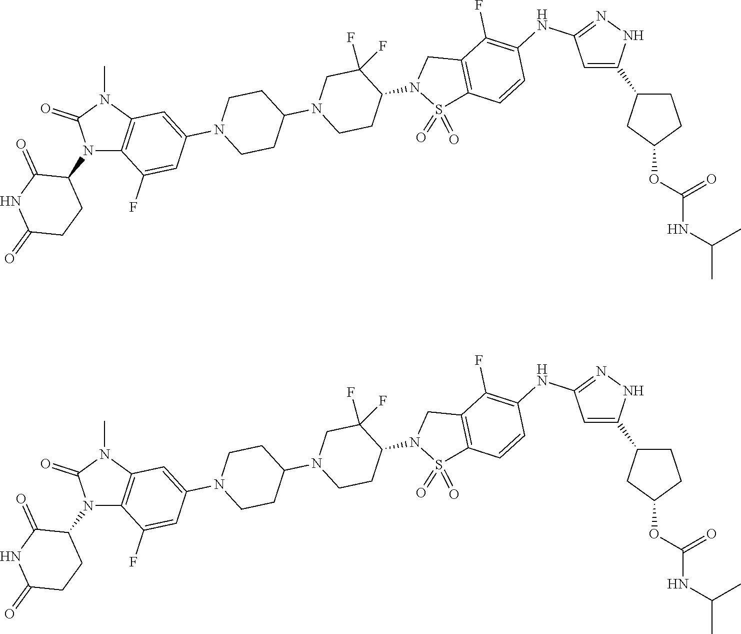 [SMILES: CC(C)NC(=O)O[C@H]1CC[C@@H](c2cc(Nc3ccc4c(c3F)CN([C@@H]3CCN(C5CCN(c6cc(F)c7c(c6)n(C)c(=O)n7[C@H]6CCC(=O)NC6=O)CC5)CC3(F)F)S4(=O)=O)n[nH]2)C1 ; CC(C)NC(=O)O[C@H]1CC[C@@H](c2cc(Nc3ccc4c(c3F)CN([C@@H]3CCN(C5CCN(c6cc(F)c7c(c6)n(C)c(=O)n7[C@@H]6CCC(=O)NC6=O)CC5)CC3(F)F)S4(=O)=O)n[nH]2)C1]

The titled compound was synthesized in the procedures similar to Example 60 in a racemate form, which was further separated by chiral HPLC to give:

Enantiomer 1 (Example 244, 100% ee); Retention time: 6.466 min. ¹H NMR (500 MHz, DMSO) δ 12.01 (s, 1H), 11.07 (s, 1H), 8.82 (s, 1H), 8.33 (s, 1H), 7.58 (d, J=8.6 Hz, 1H), 6.95 (s, 1H), 6.70 (s, 1H), 6.54 (d, J=16.0 Hz, 1H), 5.84 (s, 1H), 5.40 (s, 1H), 5.06-4.96 (m, 1H), 4.74 (d, J=14.3 Hz, 1H), 4.53 (d, J=13.4 Hz, 1H), 4.06-3.90 (m, 1H), 3.76-3.70 (m, 2H), 3.60-3.54 (m, 2H), 3.24-3.16 (m, 2H), 3.10-2.96 (m, 4H), 2.80-2.70 (m, 8H), 2.30-2.03 (s, 4H), 1.97-1.87 (m, 1H), 1.86-1.66 (m, 5H), 1.65-1.50 (m, 3H), 1.03 (d, J=6.5 Hz, 6H). LC-MS (ESI): m/z [M+H]⁺=915.4;

Enantiomer 2 (Example 245, 100% ee); Retention time: 12.91 min. ¹H NMR (500 MHz, DMSO) δ 12.01 (s, 1H), 11.07 (s, 1H), 8.82 (s, 1H), 8.33 (s, 1H), 7.58 (d, J=8.6 Hz, 1H), 6.95 (s, 1H), 6.70 (s, 1H), 6.54 (d, J=16.0 Hz, 1H), 5.84 (s, 1H), 5.40 (s, 1H), 5.06-4.96 (m, 1H), 4.74 (d, J=14.3 Hz, 1H), 4.53 (d, J=13.4 Hz, 1H), 4.06-3.90 (m, 1H), 3.76-3.70 (m, 2H), 3.60-3.54 (m, 2H), 3.24-3.16 (m, 2H), 3.10-2.96 (m, 4H), 2.80-2.70 (m, 8H), 2.30-2.03 (s, 4H), 1.97-1.87 (m, 1H), 1.86-1.66 (m, 5H), 1.65-1.50 (m, 3H), 1.03 (d, J=6.5 Hz, 6H). LC-MS (ESI): m/z [M+H]⁺=915.4;

Chiral analytical method: Column: ICHIRALPAK SA 4.6*150 mm 5 μm; Mobile phase: A for MtBE (0.1%2M NH3 MeOH) (%) and B for MeOH:DCM=50:50(%); Gradient: Mobile Phase A: Mobile Phase B=30:70 (v/v); HPLC Equipment: HPLC-Agilent, Back pressure: 100 bar; Column temperature: 35° C.

Chiral Prep-HPLC Condition: CHIRALPAK SA 250*20.0 mml, 5 μm; Mobile phase: A for MtBE (0.1%2M NH3 MeOH) (%) and B for MeOH:DCM=50:50; Gradient: Mobile Phase A: Mobile Phase B=30:70 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 246 and 247: (1S,3R)-3-(3-((2-((S)-1′-(1-((S)-2,6-dioxopiperidin-3-yl)-7-fluoro-3-methyl-2-oxo-2,3-dihydro-1H-benzo[d]imidazol-5-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropyl carbamate and (1S,3R)-3-(3-((2-((S)-1′-(1-((R)-2,6-dioxopiperidin-3-yl)-7-fluoro-3-methyl-2-oxo-2,3-dihydro-1H-benzo[d]imidazol-5-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropyl carbamate

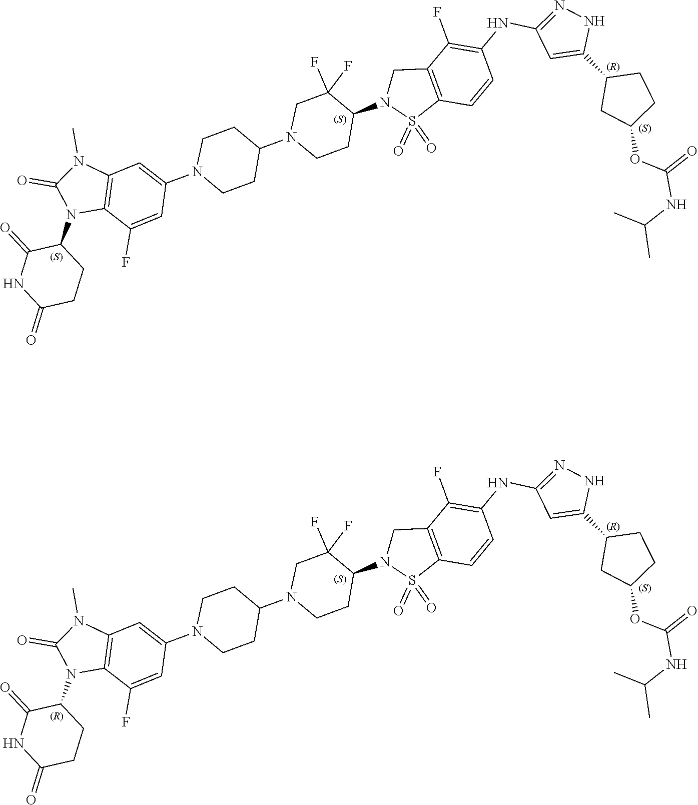

The titled compound was synthesized in the procedures similar to Example 60 in a racemate form, which was further separated by chiral HPLC to give:

Enantiomer 1 (Example 246, 100% ee); Retention time: 7.315 min. ¹H NMR (500 MHz, DMSO) δ 12.01 (s, 1H), 11.08 (s, 1H), 8.82 (s, 1H), 8.32 (t, 1H), 7.58 (d, J=8.6 Hz, 1H), 6.94 (d, J=7.2 Hz, 1H), 6.70 (s, 1H), 6.54 (d, J=14.5 Hz, 1H), 5.84 (s, 1H), 5.41 (s, 1H), 5.00 (s, 1H), 4.74 (d, J=14.3 Hz, 1H), 4.53 (d, J=13.6 Hz, 1H), 4.05-3.91 (m, 2H), 3.75-3.66 (m, 4H), 3.59-3.55 (m, 3H), 3.10-2.95 (m, 4H), 2.70-2.62 (m, 6H), 2.16-2.00 (m, 3H), 1.93-1.70 (m, 6H), 1.57 (s, 3H), 1.03 (d, J=5.8 Hz, 6H). LC-MS (ESI): m/z [M+H]⁺=915.30.

Enantiomer 2 (Example 247, 100% ee); Retention time: 9.761 min. ¹H NMR (500 MHz, DMSO) δ 12.01 (s, 1H), 11.08 (s, 1H), 8.82 (s, 1H), 8.32 (t, 1H), 7.58 (d, J=8.6 Hz, 1H), 6.94 (d, J=7.2 Hz, 1H), 6.70 (s, 1H), 6.54 (d, J=14.5 Hz, 1H), 5.84 (s, 1H), 5.41 (s, 1H), 5.00 (s, 1H), 4.74 (d, J=14.3 Hz, 1H), 4.53 (d, J=13.6 Hz, 1H), 4.05-3.91 (m, 2H), 3.75-3.66 (m, 4H), 3.59-3.55 (m, 3H), 3.10-2.95 (m, 4H), 2.70-2.62 (m, 6H), 2.16-2.00 (m, 3H), 1.93-1.70 (m, 6H), 1.57 (s, 3H), 1.03 (d, J=5.8 Hz, 6H). LC-MS (ESI): m/z [M+H]⁺=914.94.

Chiral analytical method: Column: i-Cellulose-5 4.6*250 mm 5 μm Mobile phase: A for MtBE (0.1%2M NH3 MeOH) (%) and B for MeOH:DCM=50:50 (v/v); Gradient: Mobile Phase A: Mobile Phase B=10:90 (v/v); HPLC Equipment: Agilent 1260 Infinity II (ARD_LC-808); Column temperature: 25° C.

Chiral Prep-HPLC Condition: i-Cellulose-5 4.6*250 mm 5 μm Mobile phase: A for MtBE (0.1%2M NH3 MeOH)(%) and B for MeOH:DCM=50:50; Gradient: Mobile Phase A: Mobile Phase B=10:90 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 248 and 249: (1S,3R)-3-(3-((2-((R)-1′-(1-((S)-2,6-dioxopiperidin-3-yl)-7-fluoro-3-isopropyl-2-oxo-2,3-dihydro-1H-benzo[d]imidazol-5-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-((R)-1′-(1-((R)-2,6-dioxopiperidin-3-yl)-7-fluoro-3-isopropyl-2-oxo-2,3-dihydro-1H-benzo[d]imidazol-5-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

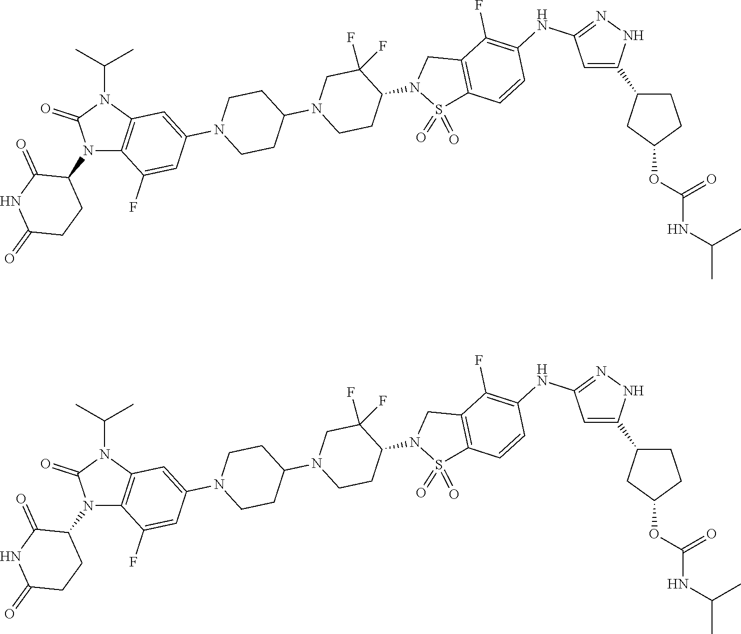 [SMILES: CC(C)NC(=O)O[C@H]1CC[C@@H](c2cc(Nc3ccc4c(c3F)CN([C@@H]3CCN(C5CCN(c6cc(F)c7c(c6)n(C(C)C)c(=O)n7[C@H]6CCC(=O)NC6=O)CC5)CC3(F)F)S4(=O)=O)n[nH]2)C1 ; CC(C)NC(=O)O[C@H]1CC[C@@H](c2cc(Nc3ccc4c(c3F)CN([C@@H]3CCN(C5CCN(c6cc(F)c7c(c6)n(C(C)C)c(=O)n7[C@@H]6CCC(=O)NC6=O)CC5)CC3(F)F)S4(=O)=O)n[nH]2)C1]

### Step 1: 3-(7-fluoro-3-methyl-2-oxo-5-(4-oxopiperidin-1-yl)-2,3-dihydro-1H-benzo[d]imidazol-1-yl) piperidine-2,6-dione

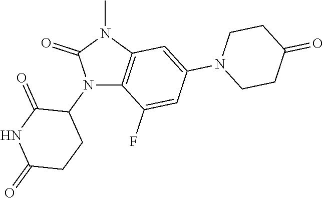 [SMILES: Cn1c(=O)n(C2CCC(=O)NC2=O)c2c(F)cc(N3CCC(=O)CC3)cc21]

The titled compound was synthesized in the procedures similar to Example 64, LC-MS (ESI): m/z [M+H]+=375.3;

### Step 2: (1S,3R)-3-(3-((2-((R)-1′-(1-((S)-2,6-dioxopiperidin-3-yl)-7-fluoro-3-isopropyl-2-oxo-2,3-dihydro-1H-benzo[d]imidazol-5-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-((R)-1′-(1-((R)-2,6-dioxopiperidin-3-yl)-7-fluoro-3-isopropyl-2-oxo-2,3-dihydro-1H-benzo[d]imidazol-5-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

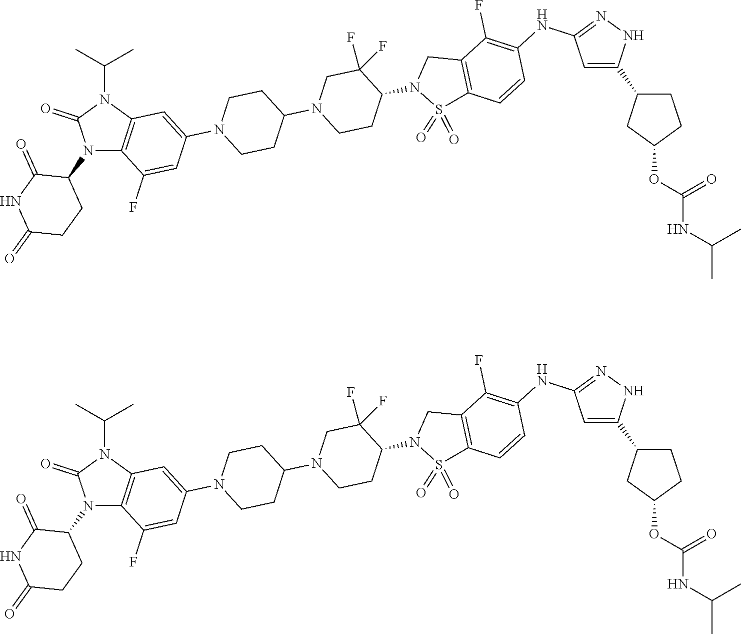 [SMILES: CC(C)NC(=O)O[C@H]1CC[C@@H](c2cc(Nc3ccc4c(c3F)CN([C@@H]3CCN(C5CCN(c6cc(F)c7c(c6)n(C(C)C)c(=O)n7[C@H]6CCC(=O)NC6=O)CC5)CC3(F)F)S4(=O)=O)n[nH]2)C1 ; CC(C)NC(=O)O[C@H]1CC[C@@H](c2cc(Nc3ccc4c(c3F)CN([C@@H]3CCN(C5CCN(c6cc(F)c7c(c6)n(C(C)C)c(=O)n7[C@@H]6CCC(=O)NC6=O)CC5)CC3(F)F)S4(=O)=O)n[nH]2)C1]

The titled compound was synthesized in the procedures similar to Example 60 in a racemate form, which was further separated by chiral HPLC to give:

Enantiomer 1 (Example 248, 100% ee); Retention time: 5.3 min. ¹H NMR (500 MHz, DMSO) δ 12.03 (s, 1H), 11.11 (s, 1H), 8.82 (s, 1H), 8.31 (s, 1H), 7.61 (t, J=15.0 Hz, 1H), 6.93 (s, 1H), 6.87 (d, J=8.4 Hz, 1H), 6.76 (s, 1H), 5.84 (s, 1H), 5.34 (dd, J=12.8, 5.2 Hz, 1H), 5.06-4.96 (m, 1H), 4.75 (d, J=14.3 Hz, 1H), 4.54 (d, J=14.8 Hz, 1H), 4.30-4.20 (m, 2H), 4.16-4.04 (m, 3H), 3.57 (s, 4H), 3.35-3.30 (m, 3H), 3.12-3.02 (m, 2H), 2.94-2.83 (m, 2H), 2.76-2.71 (m, 5H), 2.44-2.41 (m, 1H), 2.24-2.12 (m, 2H), 2.08-1.96 (m, 5H), 1.94-1.82 (m, 3H), 1.80-1.66 (m, 3H), 1.64-1.44 (m, 4H), 1.03 (d, J=6.3 Hz, 6H). LC-MS (ESI): m/z [M+H]⁺=943.4;

Enantiomer 2 (Example 249, 100% ee); Retention time: 7.62 min. ¹H NMR (500 MHz, DMSO) δ 12.03 (s, 1H), 11.11 (s, 1H), 8.82 (s, 1H), 8.31 (s, 1H), 7.61 (t, J=15.0 Hz, 1H), 6.93 (s, 1H), 6.87 (d, J=8.4 Hz, 1H), 6.76 (s, 1H), 5.84 (s, 1H), 5.34 (dd, J=12.8, 5.2 Hz, 1H), 5.06-4.96 (m, 1H), 4.75 (d, J=14.3 Hz, 1H), 4.54 (d, J=14.8 Hz, 1H), 4.30-4.20 (m, 2H), 4.16-4.04 (m, 3H), 3.57 (s, 4H), 3.35-3.30 (m, 3H), 3.12-3.02 (m, 2H), 2.94-2.83 (m, 2H), 2.76-2.71 (m, 5H), 2.44-2.41 (m, 1H), 2.24-2.12 (m, 2H), 2.08-1.96 (m, 5H), 1.94-1.82 (m, 3H), 1.80-1.66 (m, 3H), 1.64-1.44 (m, 4H), 1.03 (d, J=6.3 Hz, 6H). LC-MS (ESI): m/z [M+H]⁺=943.4;

Chiral analytical method: Column: ICHIRALPAK IC-5 4.6*150 mm 5 μm; Mobile phase: A for MtBE (0.1%2M NH3 MeOH) (%) and B for MeOH:DCM=50:50(%); Gradient: Mobile Phase A: Mobile Phase B=10:90 (v/v); HPLC Equipment: HPLC-Agilent, Back pressure: 100 bar; Column temperature: 35° C.

Chiral Prep-HPLC Condition: CHIRALPAK IC-5 250*20.0 mml, 5 μm; Mobile phase: A for MtBE (0.1%2M NH3 MeOH) (%) and B for MeOH:DCM=50:50; Gradient: Mobile Phase A: Mobile Phase B=10:90 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 250: (1S,3R)-3-(3-((2-((4S)-1-((1-(1-(2,6-dioxopiperidin-3-yl)-3,6-dimethyl-2-oxo-2,3-dihydro-1H-benzo[d]imidazol-5-yl)piperidin-4-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl isopropylcarbamate

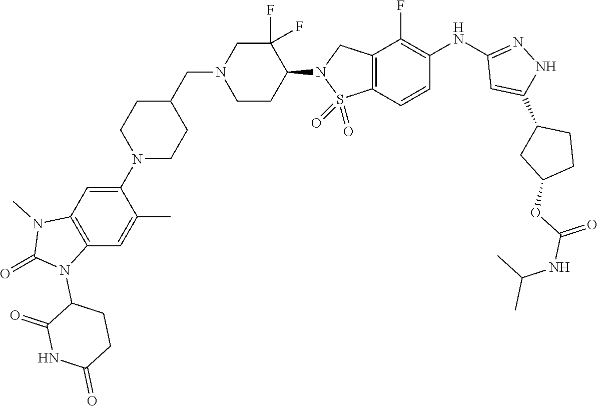 [SMILES: Cc1cc2c(cc1N1CCC(CN3CC[C@H](N4Cc5c(ccc(Nc6cc([C@@H]7CC[C@H](OC(=O)NC(C)C)C7)[nH]n6)c5F)S4(=O)=O)C(F)(F)C3)CC1)n(C)c(=O)n2C1CCC(=O)NC1=O]

### Step 1: 1-(1-(2,6-dioxopiperidin-3-yl)-3,6-dimethyl-2-oxo-2,3-dihydro-1H-benzo[d]imidazol-5-yl)piperidine-4-carbaldehyde

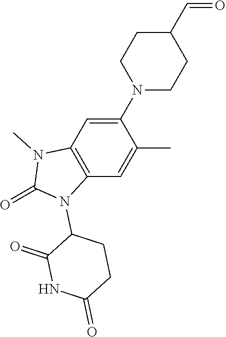 [SMILES: Cc1cc2c(cc1N1CCC(C=O)CC1)n(C)c(=O)n2C1CCC(=O)NC1=O]

The titled compound was synthesized in the procedures similar to Example 140. LC-MS (ESI): m/z [M+H]⁺=385.6.

### Step 2: (1S,3R)-3-(3-((2-((4S)-1-((1-(1-(2,6-dioxopiperidin-3-yl)-3,6-dimethyl-2-oxo-2,3-dihydro-1H-benzo[d]imidazol-5-yl)piperidin-4-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl isopropylcarbamate

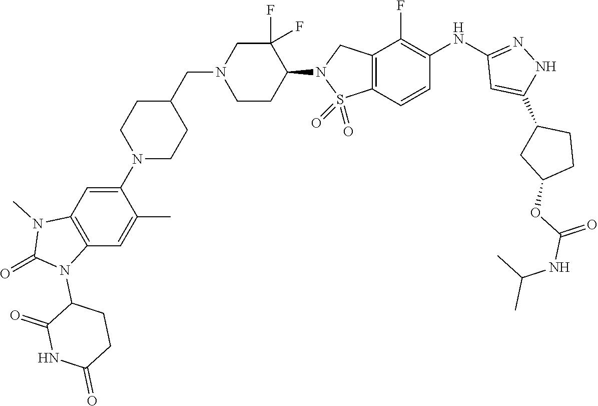 [SMILES: Cc1cc2c(cc1N1CCC(CN3CC[C@H](N4Cc5c(ccc(Nc6cc([C@@H]7CC[C@H](OC(=O)NC(C)C)C7)[nH]n6)c5F)S4(=O)=O)C(F)(F)C3)CC1)n(C)c(=O)n2C1CCC(=O)NC1=O]

The titled compound was synthesized in the procedures similar to Example 42. ¹H NMR (500 MHz, DMSO) δ 12.24-11.80 (m, 1H), 11.06 (s, 1H), 8.83 (s, 1H), 8.31 (s, 1H), 7.60 (d, J=8.8 Hz, 1H), 7.05-6.87 (m, 3H), 5.84 (s, 1H), 5.30 (s, 1H), 5.00 (s, 1H), 4.78-4.75 (m, 1H), 4.57-4.55 (m, 1H), 3.60-3.56 (m, 2H), 3.32 (s, 4H), 3.07-2.88 (m, 5H), 2.73-2.54 (m, 8H), 2.27 (s, 4H), 2.06-1.82 (m, 7H), 1.71 (s, 3H), 1.60 (s, 1H), 1.35 (s, 2H), 1.03-1.02 (m, 6H). LC-MS (ESI): m/z [M+H]⁺=925.33.

### Example 251: (1S,3R)-3-(3-((2-((4R)-1-((1-(1-(2,6-dioxopiperidin-3-yl)-3,6-dimethyl-2-oxo-2,3-dihydro-1H-benzo[d]imidazol-5-yl)piperidin-4-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl isopropylcarbamate

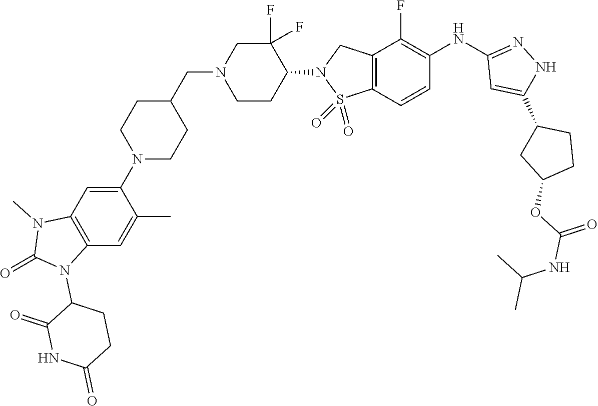 [SMILES: Cc1cc2c(cc1N1CCC(CN3CC[C@@H](N4Cc5c(ccc(Nc6cc([C@@H]7CC[C@H](OC(=O)NC(C)C)C7)[nH]n6)c5F)S4(=O)=O)C(F)(F)C3)CC1)n(C)c(=O)n2C1CCC(=O)NC1=O]

The titled compound was synthesized in the procedures similar to Example 250. ¹H NMR (500 MHz, DMSO) δ 12.07 (s, 1H), 11.07 (s, 1H), 8.84 (s, 1H), 8.32 (t, J=7.6 Hz, 1H), 7.60 (d, J=8.7 Hz, 1H), 7.12-6.90 (m, 2H), 5.84 (s, 1H), 5.32 (s, 1H), 5.00 (s, 1H), 4.76 (s, 1H), 4.57-4.55 (m, 2H), 3.61-3.54 (m, 2H), 3.33 (s, 3H), 3.16-2.99 (m, 3H), 2.93-2.87 (m, 2H), 2.79-2.68 (m, 2H), 2.65-2.56 (m, 3H), 2.54-2.52 (m, 6H), 2.46 (s, 2H), 2.29 (s, 3H), 2.04-1.96 (m, 3H), 1.94-1.79 (m, 4H), 1.72 (s, 2H), 1.60 (s, 1H), 1.42 (s, 1H), 1.12-0.96 (m, 7H). LC-MS (ESI): m/z [M+H]⁺=925.64.

### Example 252: (1S,3R)-3-(3-((2-((4S)-1-((1-(1-(2,6-dioxopiperidin-3-yl)-2-oxo-1,2,5,6-tetrahydro-4H-imidazo[4,5,1-ij]quinolin-7-yl)piperidin-4-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl isopropylcarbamate

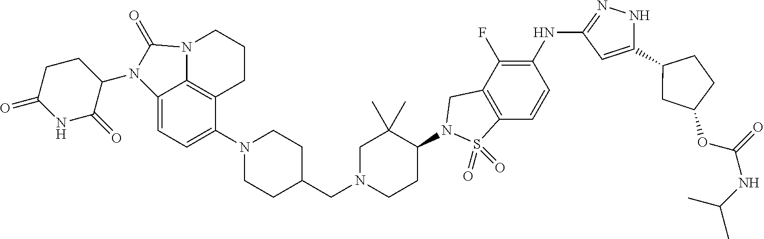 [SMILES: CC(C)NC(=O)O[C@H]1CC[C@@H](c2cc(Nc3ccc4c(c3F)CN([C@H]3CCN(CC5CCN(c6ccc7c8c6CCCn8c(=O)n7C6CCC(=O)NC6=O)CC5)CC3(C)C)S4(=O)=O)n[nH]2)C1]

### Step 1: 3-(7-(4-(dimethoxymethyl)piperidin-1-yl)-2-oxo-5,6-dihydro-4H-imidazo[4,5,1-ij]quinolin-1(2H)-yl)piperidine-2,6-dione

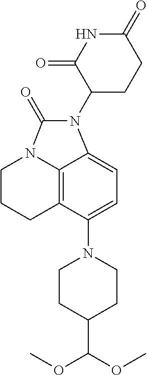 [SMILES: COC(OC)C1CCN(c2ccc3c4c2CCCn4c(=O)n3C2CCC(=O)NC2=O)CC1]

To a solution of 3-(7-bromo-2-oxo-5,6-dihydro-4H-imidazo[4,5,1-ij]quinolin-1(2H)-yl)piperidine-2,6-dione (700 mg, 1.9 mmol, 1.0 eq) and 4-(dimethoxymethyl)piperidine (918 mg, 5.8 mmol, 3.0 eq) in 1,4-dioxane (20 mL) was added Cs₂CO₃ (945 mg, 1.9 mmol, 1.5 eq) and Palladium [1,3-bis[2,6-bis(1-ethylpropyl)phenyl]-4,5-dichloro-1,3-dihydro-2H-imidazol-2-ylidene]dichloro(2-methylpyridine)-, (SP-4-1)-(ACI) (81 mg, 0.096 mmol, 0.05 eq) under N₂, the mixture was stirred at 100° C. for 5 h under N₂. After completion, the mixture was cooled, diluted with water (20 mL) and extracted with EtOAc (30 mL×3), the combined organic phase was washed with brine (20 mL×2), dried over Na₂SO₄ and concentrated to give a residue, which was purified by silica gel column chromatography (Petroleum ether/EtOAc=1:1) to give a crude product. Then the crude product was purified by reversed-phase chromatography (acetonitrile in water, 30% to 35%) to give the product (250 mg, 29.4%) as a yellow solid.

### Step 2: 1-(1-(2,6-dioxopiperidin-3-yl)-2-oxo-1,2,5,6-tetrahydro-4H-imidazo[4,5,1-ij]quinolin-7-yl)piperidine-4-carbaldehyde

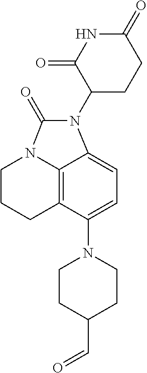 [SMILES: O=CC1CCN(c2ccc3c4c2CCCn4c(=O)n3C2CCC(=O)NC2=O)CC1]

To a flask was added 3-(7-(4-(dimethoxymethyl)piperidin-1-yl)-2-oxo-5,6-dihydro-4H-imidazo[4,5,1-ij]quinolin-1(2H)-yl)piperidine-2,6-dione (200 mg, 0.45 mmol) and formic acid (5 mL). The resulting solution was stirred for 2 h at room temperature. The filtrate was concentrated under vacuum. The residue was the crude product. MS (ESI) m/e [M+1]⁺=397.4

### Step 3: (1S,3R)-3-(3-((2-((4S)-1-((1-(1-(2,6-dioxopiperidin-3-yl)-2-oxo-1,2,5,6-tetrahydro-4H-imidazo[4,5,1-ij]quinolin-7-yl)piperidin-4-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl isopropylcarbamate

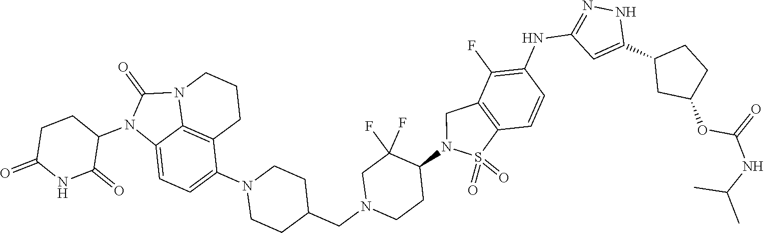 [SMILES: CC(C)NC(=O)O[C@H]1CC[C@@H](c2cc(Nc3ccc4c(c3F)CN([C@H]3CCN(CC5CCN(c6ccc7c8c6CCCn8c(=O)n7C6CCC(=O)NC6=O)CC5)CC3(F)F)S4(=O)=O)n[nH]2)C1]

The titled compound was synthesized in the procedures similar to Example 180. LC-MS (ESI): m/z [M+H]⁺=937.49; ¹H NMR (500 MHz, DMSO) δ=12.01 (s, 1H), 11.05 (s, 1H), 8.82 (s, 1H), 8.33 (t, J=7.9, 1H), 7.58 (d, J=8.7, 1H), 6.94 (d, J=7.2, 1H), 6.82 (d, J=8.3, 1H), 6.62 (d, J=8.4, 1H), 5.84 (s, 1H), 5.27 (dd, J=12.9, 5.4, 1H), 5.00 (s, 1H), 4.75 (d, J=14.3, 1H), 4.54 (d, J=13.1, 1H), 4.00 (dd, J=25.3, 12.2, 1H), 3.73 (s, 2H), 3.58 (m, 1H), 3.18 (m, 1H), 3.08 (m, 3H), 2.98-2.87 (m, 2H), 2.72 (m, 2H), 2.69-2.65 (m, 1H), 2.64-2.61 (m, 2H), 2.58 (m, 3H), 2.45 (m, 1H), 2.32 (m, 2H), 2.20 (m, 1H), 2.05-1.94 (m, 4H), 1.94-1.87 (m, 1H), 1.80 (m, 3H), 1.71 (m, 2H), 1.62 (m, 2H), 1.26 (m, 2H), 1.03 (m, 6H).

### Example 253: (1S,3R)-3-(3-((2-((4R)-1′-(1-(2,6-dioxopiperidin-3-yl)-2-oxo-1,2,5,6-tetrahydro-4H-imidazo[4,5,1-ij]quinolin-7-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

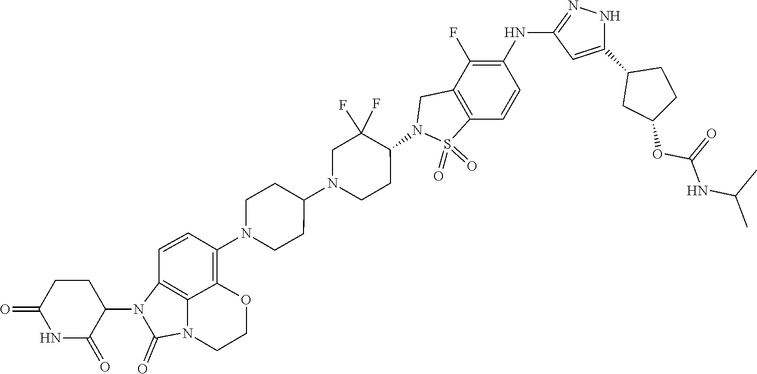 [SMILES: CC(C)NC(=O)O[C@H]1CC[C@@H](c2cc(Nc3ccc4c(c3F)CN([C@@H]3CCN(C5CCN(c6ccc7c8c6OCCn8c(=O)n7C6CCC(=O)NC6=O)CC5)CC3(F)F)S4(=O)=O)n[nH]2)C1]

The titled compound was synthesized in the procedures similar to Example 181. ¹H NMR (500 MHz, DMSO-d₆) δ 12.01 (s, 1H), 11.06 (s, 1H), 8.82 (s, 1H), 8.38-8.26 (m, 1H), 7.58 (d, J=10 Hz, 1H), 6.99-6.91 (m, 1H), 6.85-6.78 (m, 1H), 6.66-6.58 (m, 1H), 5.84 (s, 1H), 5.33-5.24 (m, 1H), 5.07-4.95 (m, 1H), 4.80-4.69 (m, 1H), 4.60-4.59 (m, 1H), 4.04-3.91 (m, 1H), 3.79-3.66 (m, 2H), 3.62-3.52 (m, 1H), 3.27-3.19 (m, 1H), 3.18-3.11 (m, 2H), 3.10-2.98 (m, 2H), 2.96-2.85 (m, 1H), 2.83-2.70 (m, 4H), 2.69-2.57 (m, 4H), 2.54 (s, 2H), 2.48-2.45 (m, 1H), 2.20-2.08 (m, 1H), 2.07-1.94 (m, 4H), 1.93-1.77 (m, 4H), 1.76-1.55 (m, 5H), 1.07-0.99 (m, 6H). LC-MS (ESI): m/z [M+H]⁺=923.

### Example 254: (1S,3R)-3-(3-((2-((4S)-1-((1-(1-(2,6-dioxopiperidin-3-yl)-2-oxo-1,2,3,4-tetrahydro-5-oxa-1,2a-diazaacenaphthylen-6-yl)piperidin-4-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl isopropylcarbamate

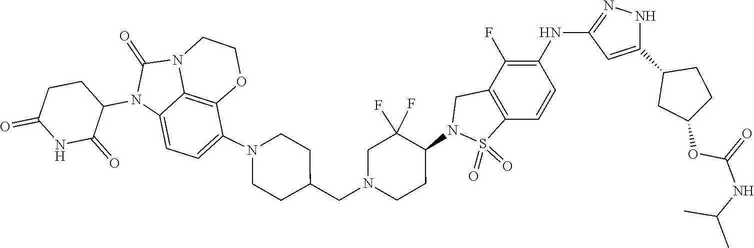 [SMILES: CC(C)NC(=O)O[C@H]1CC[C@@H](c2cc(Nc3ccc4c(c3F)CN([C@H]3CCN(CC5CCN(c6ccc7c8c6OCCn8c(=O)n7C6CCC(=O)NC6=O)CC5)CC3(F)F)S4(=O)=O)n[nH]2)C1]

The titled compound was synthesized in the procedures similar to Example 227. LC-MS (ESI): m/z [M+H]⁺=939.57; ¹H NMR (500 MHz, DMSO) δ=12.01 (s, 1H), 11.07 (s, 1H), 8.82 (s, 1H), 8.33 (t, J=8.0, 1H), 7.58 (d, J=8.6, 1H), 6.94 (d, J=7.6, 1H), 6.60 (d, J=8.4, 1H), 6.53 (d, J=8.4, 1H), 5.84 (s, 1H), 5.27 (dd, J=13.1, 5.3, 1H), 5.00 (s, 1H), 4.75 (d, J=14.4, 1H), 4.54 (d, J=13.3, 1H), 4.38 (t, J=4.5, 2H), 3.99 (dd, J=25.2, 12.4, 1H), 3.92 (d, J=4.5, 2H), 3.61-3.55 (m, 1H), 3.16 (m, 1H), 3.10-3.01 (m, 1H), 2.91 (m, 2H), 2.70-2.66 (m, 1H), 2.62 (m, 1H), 2.60-2.56 (m, 4H), 2.48-2.43 (m, 1H), 2.32 (m, 3H), 2.24-2.15 (m, 1H), 2.01 (m, 2H), 1.95-1.87 (m, 1H), 1.76 (m, 6H), 1.61 (s, 2H), 1.33-1.22 (m, 2H), 1.03 (m, 6H).

### Example 255: (1S,3R)-3-(3-((2-((4S)-1′-(1-(2,6-dioxopiperidin-3-yl)-2-oxo-1,2,3,4-tetrahydro-5-oxa-1,2a-diazaacenaphthylen-6-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

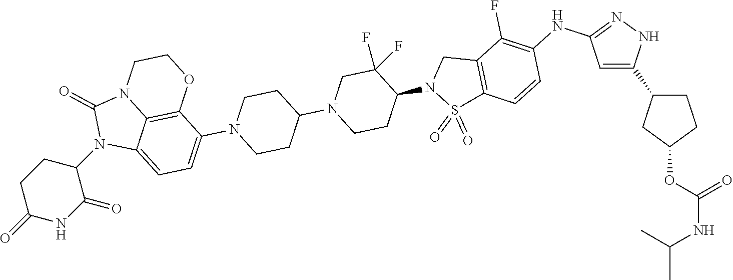 [SMILES: CC(C)NC(=O)O[C@H]1CC[C@@H](c2cc(Nc3ccc4c(c3F)CN([C@H]3CCN(C5CCN(c6ccc7c8c6OCCn8c(=O)n7C6CCC(=O)NC6=O)CC5)CC3(F)F)S4(=O)=O)n[nH]2)C1]

The titled compound was synthesized in the procedures similar to Example 179. LC-MS (ESI): m/z [M+H]⁺=925.59; ¹H NMR (500 MHz, DMSO) δ=12.01 (s, 1H), 11.07 (s, 1H), 8.82 (s, 1H), 8.33 (s, 1H), 7.58 (d, J=8.6, 1H), 6.94 (d, J=6.1, 1H), 6.60 (d, J=8.4, 1H), 6.53 (d, J=8.4, 1H), 5.84 (s, 1H), 5.27 (dd, J=12.9, 5.3, 1H), 5.00 (s, 1H), 4.74 (d, J=14.4, 1H), 4.54 (d, J=13.3, 1H), 4.39 (s, 2H), 3.97 (m, 1H), 3.92 (m, 2H), 3.21 (m, 1H), 3.07 (m, 1H), 3.01 (m, 1H), 2.92 (m, 1H), 2.78 (m, 1H), 2.72 (m, 1H), 2.67 (m, 2H), 2.64 (m, 3H), 2.59 (m, 3H), 2.47-2.43 (m, 1H), 2.13 (m, 1H), 2.01 (m, 2H), 1.91 (m, 1H), 1.79 (m, 3H), 1.72 (m, 2H), 1.61 (m, 3H), 1.03 (m, 6H).

### Example 256 and 257: (1S,3R)-3-(3-((2-((R)-1-((1-(1-((S)-2,6-dioxopiperidin-3-yl)-4,7-difluoro-3-methyl-2-oxo-2,3-dihydro-1H-benzo[d]imidazol-5-yl)piperidin-4-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-((R)-1-((1-(1-((R)-2,6-dioxopiperidin-3-yl)-4,7-difluoro-3-methyl-2-oxo-2,3-dihydro-1H-benzo[d]imidazol-5-yl)piperidin-4-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl isopropylcarbamate

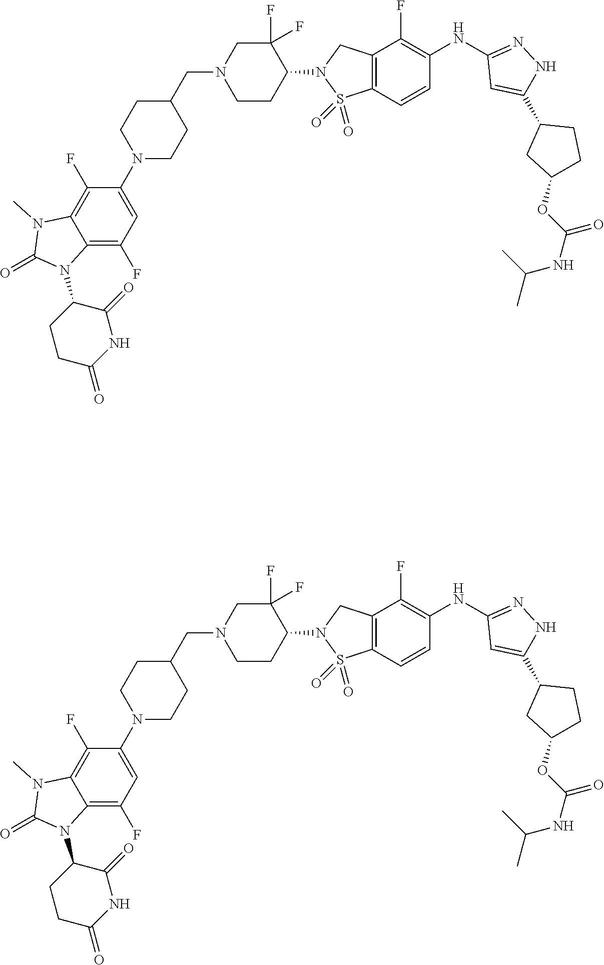 [SMILES: CC(C)NC(=O)O[C@H]1CC[C@@H](c2cc(Nc3ccc4c(c3F)CN([C@@H]3CCN(CC5CCN(c6cc(F)c7c(c6F)n(C)c(=O)n7[C@H]6CCC(=O)NC6=O)CC5)CC3(F)F)S4(=O)=O)n[nH]2)C1 ; CC(C)NC(=O)O[C@H]1CC[C@@H](c2cc(Nc3ccc4c(c3F)CN([C@@H]3CCN(CC5CCN(c6cc(F)c7c(c6F)n(C)c(=O)n7[C@@H]6CCC(=O)NC6=O)CC5)CC3(F)F)S4(=O)=O)n[nH]2)C1]

The titled compound was synthesized in the procedures similar to Example 55 in a racemate form, which was further separated by chiral HPLC to give:

Enantiomer 1 (Example 256, 100% ee); Retention time: 6.5 min. ¹H NMR (500 MHz, DMSO) δ 12.00 (s, 1H), 11.11 (s, 1H), 8.82 (s, 1H), 8.33 (t, J=8.1 Hz, 1H), 7.58 (d, J=8.6 Hz, 1H), 6.94 (d, J=7.7 Hz, 1H), 6.67 (s, 1H), 5.84 (s, 1H), 5.46 (s, 1H), 5.00 (s, 1H), 4.76-4.74 (m, 1H), 4.55-4.52 (m, 1H), 4.03-3.96 (m, 1H), 3.57 (s, 1H), 3.50 (s, 3H), 3.27-3.25 (m, 1H), 3.17 (s, 1H), 3.07 (s, 1H), 2.99-2.90 (m, 2H), 2.63 (s, 3H), 2.61-2.54 (m, 3H), 2.36-2.33 (m, 2H), 2.21-2.16 (m, 2H), 2.10 (s, 1H), 2.04-2.02 (m, 1H), 1.91 (s, 1H), 1.80 (s, 3H), 1.71 (s, 2H), 1.62 (s, 1H), 1.37-1.22 (m, 2H), 1.19-1.12 (m, 2H), 1.03-1.02 (m, 6H). LC-MS (ESI): m/z [M+H]⁺=947.86.

Enantiomer 2 (Example 257, 100% ee); Retention time: 9.64 min. ¹H NMR (500 MHz, DMSO) δ 12.00 (s, 1H), 11.11 (s, 1H), 8.82 (s, 1H), 8.33 (t, J=8.1 Hz, 1H), 7.58 (d, J=8.6 Hz, 1H), 6.94 (d, J=7.7 Hz, 1H), 6.67 (s, 1H), 5.84 (s, 1H), 5.46 (s, 1H), 5.00 (s, 1H), 4.76-4.74 (m, 1H), 4.55-4.52 (m, 1H), 4.03-3.96 (m, 1H), 3.57 (s, 1H), 3.50 (s, 3H), 3.27-3.25 (m, 1H), 3.17 (s, 1H), 3.07 (s, 1H), 2.99-2.90 (m, 2H), 2.63 (s, 3H), 2.61-2.54 (m, 3H), 2.36-2.33 (m, 2H), 2.21-2.16 (m, 2H), 2.10 (s, 1H), 2.04-2.02 (m, 1H), 1.91 (s, 1H), 1.80 (s, 3H), 1.71 (s, 2H), 1.62 (s, 1H), 1.37-1.22 (m, 2H), 1.19-1.12 (m, 2H), 1.03-1.02 (m, 6H). LC-MS (ESI): m/z [M+H]⁺=947.86.

Chiral analytical method: Column: I-Cellulose-5, 4.6 mm×250 mm, 5 um; Mobile phase: A for Hexane and B for EtOH (0.1% 2M NH₃ MeOH); Gradient: Mobile Phase A: Mobile Phase B=30:70 (v/v); HPLC Equipment: HPLC-Agilent, Back pressure: 100 bar; Column temperature: 35° C.

Chiral Prep-HPLC Condition: Column: I-Cellulose-5, 21.2 mm×250 mm, 5 um; Mobile phase: A for Hexane and B for EtOH (0.2% 2M NH₃ MeOH); Gradient: Mobile Phase A: Mobile Phase B=30:70 (v/v); Flow Rate: 20 mL/min, Wave Length: UV 200 nm and 270 nm, Prep-HPLC Equipment: Prep-HPLC-Gilson, Back pressure: 100 bar; Column temperature: 25° C.

### Example 258 and 259: (1S,3R)-3-(3-((2-((R)-1′-(1-((S)-2,6-dioxopiperidin-3-yl)-4,7-difluoro-3-methyl-2-oxo-2,3-dihydro-1H-benzo[d]imidazol-5-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-((R)-1′-(1-((R)-2,6-dioxopiperidin-3-yl)-4,7-difluoro-3-methyl-2-oxo-2,3-dihydro-1H-benzo[d]imidazol-5-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

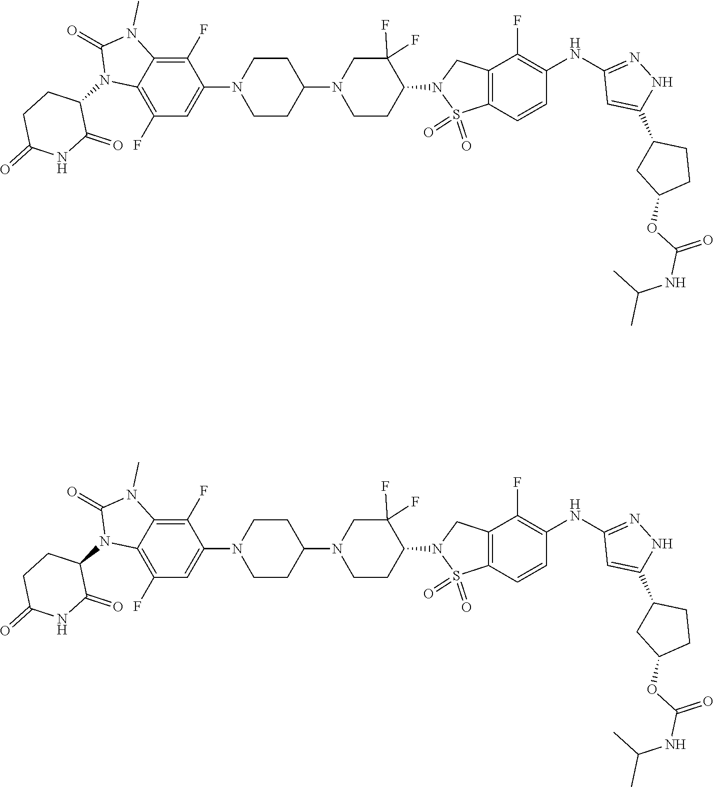 [SMILES: CC(C)NC(=O)O[C@H]1CC[C@@H](c2cc(Nc3ccc4c(c3F)CN([C@@H]3CCN(C5CCN(c6cc(F)c7c(c6F)n(C)c(=O)n7[C@H]6CCC(=O)NC6=O)CC5)CC3(F)F)S4(=O)=O)n[nH]2)C1 ; CC(C)NC(=O)O[C@H]1CC[C@@H](c2cc(Nc3ccc4c(c3F)CN([C@@H]3CCN(C5CCN(c6cc(F)c7c(c6F)n(C)c(=O)n7[C@@H]6CCC(=O)NC6=O)CC5)CC3(F)F)S4(=O)=O)n[nH]2)C1]

Enantiomer 1 (Example 258, 100% ee); Retention time: 6.5 min. ¹H NMR (500 MHz, DMSO) δ 12.01 (s, 1H), 11.11 (s, 1H), 8.81 (s, 1H), 8.33 (s, 1H), 7.58 (d, J=8.6 Hz, 1H), 6.98-6.86 (m, 1H), 6.67 (s, 1H), 5.84 (s, 1H), 5.46 (s, 1H), 5.01 (s, 1H), 4.76-4.73 (m, 1H), 4.55-4.52 (m, 1H), 3.60-3.50 (m, 4H), 3.24-3.18 (m, 1H), 3.07-3.02 (m, 3H), 2.80-2.60 (m, 7H), 2.21-2.01 (m, 5H), 1.92-1.78 (m, 4H), 1.72-1.62 (m, 5H), 1.24 (s, 2H), 1.03-1.02 (m, 6H). LC-MS (ESI): m/z [M+H]⁺=933.39.

Enantiomer 2 (Example 259, 100% ee); Retention time: 9.64 min. ¹H NMR (500 MHz, DMSO) δ 12.01 (s, 1H), 11.11 (s, 1H), 8.81 (s, 1H), 8.33 (s, 1H), 7.58 (d, J=8.6 Hz, 1H), 6.98-6.86 (m, 1H), 6.67 (s, 1H), 5.84 (s, 1H), 5.46 (s, 1H), 5.01 (s, 1H), 4.76-4.73 (m, 1H), 4.55-4.52 (m, 1H), 3.60-3.50 (m, 4H), 3.24-3.18 (m, 1H), 3.07-3.02 (m, 3H), 2.80-2.60 (m, 7H), 2.21-2.01 (m, 5H), 1.92-1.78 (m, 4H), 1.72-1.62 (m, 5H), 1.24 (s, 2H), 1.03-1.02 (m, 6H). LC-MS (ESI): m/z [M+H]⁺=933.39.

Chiral analytical method: Column: I-Cellulose-5, 4.6 mm×250 mm, 5 um; Mobile phase: A for Hexane and B for EtOH (0.1% 2M NH₃ MeOH); Gradient: Mobile Phase A: Mobile Phase B=30:70 (v/v); HPLC Equipment: HPLC-Agilent, Back pressure: 100 bar; Column temperature: 35° C.

Chiral Prep-HPLC Condition: Column: I-Cellulose-5, 21.2 mm×250 mm, 5 um; Mobile phase: A for Hexane and B for EtOH (0.2% 2M NH₃ MeOH); Gradient: Mobile Phase A: Mobile Phase B=30:70 (v/v); Flow Rate: 20 mL/min, Wave Length: UV 200 nm and 270 nm, Prep-HPLC Equipment: Prep-HPLC-Gilson, Back pressure: 100 bar; Column temperature: 25° C.

### Example 260: (1S,3R)-3-(3-((2-((4S)-1-((1-(1-(2,6-dioxopiperidin-3-yl)-3-methyl-2-oxo-2,3-dihydro-1H-benzo[d]imidazol-5-yl)piperidin-4-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl isopropylcarbamate

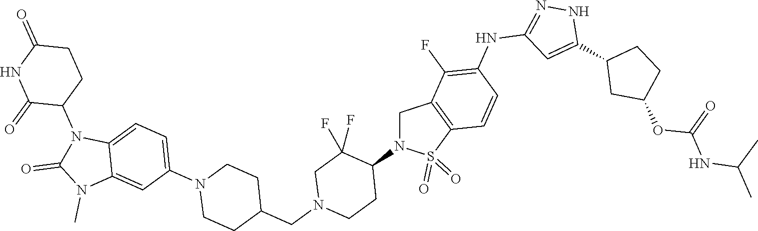 [SMILES: CC(C)NC(=O)O[C@H]1CC[C@@H](c2cc(Nc3ccc4c(c3F)CN([C@H]3CCN(CC5CCN(c6ccc7c(c6)n(C)c(=O)n7C6CCC(=O)NC6=O)CC5)CC3(F)F)S4(=O)=O)n[nH]2)C1]

The titled compound was synthesized in the procedures similar to Example 36 in a racemate form, which was further separated by chiral HPLC to give Example 260 (100% ee); Retention time: 6.3 min. ¹H NMR (500 MHz, DMSO) δ 12.01 (s, 1H), 11.06 (s, 1H), 8.82 (s, 1H), 8.34 (d, J=13.4 Hz, 1H), 7.58 (d, J=8.7 Hz, 1H), 6.93 (t, J=8.1 Hz, 2H), 6.82 (s, 1H), 6.64 (d, J=8.3 Hz, 1H), 5.84 (s, 1H), 5.29 (dd, J=12.7, 5.4 Hz, 1H), 5.06-4.96 (m, 1H), 4.75 (d, J=14.2 Hz, 1H), 4.54 (d, J=13.3 Hz, 1H), 4.10-3.95 (m, 1H), 3.59 (s, 3H), 3.22-3.14 (m, 1H), 3.11-3.02 (m, 1H), 2.99-2.84 (m, 2H), 2.74-2.66 (m, 3H), 2.60-2.54 (m, 6H), 2.47-2.43 (m, 1H), 2.34-2.29 (m, 2H), 2.24-2.14 (m, 1H), 2.11-1.87 (m, 4H), 1.85-1.76 (m, 3H), 1.76-1.54 (m, 4H), 1.32-1.19 (m, 2H), 1.03 (d, J=6.2 Hz, 6H). LC-MS (ESI): m/z [M+H]⁺=911.4;

### Example 261: (1S,3R)-3-(3-((2-((4R)-1-((1-(1-(2,6-dioxopiperidin-3-yl)-3-methyl-2-oxo-2,3-dihydro-1H-benzo[d]imidazol-5-yl)piperidin-4-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl isopropylcarbamate

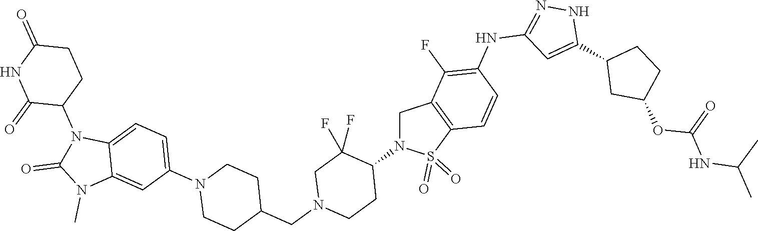 [SMILES: CC(C)NC(=O)O[C@H]1CC[C@@H](c2cc(Nc3ccc4c(c3F)CN([C@@H]3CCN(CC5CCN(c6ccc7c(c6)n(C)c(=O)n7C6CCC(=O)NC6=O)CC5)CC3(F)F)S4(=O)=O)n[nH]2)C1]

The titled compound was synthesized in the procedures similar to Example 36. ¹H NMR (500 MHz, DMSO-d₆) δ 12.01 (s, 1H), 11.12 (s, 1H), 8.82 (s, 1H), 8.33 (s, 1H), 7.58 (d, J=10 Hz, 1H), 7.35-7.28 (m, 2H), 6.94 (d, J=10 Hz, 1H), 5.62 (s, 1H), 5.48-5.37 (m, 1H), 5.02-4.95 (m, 1H), 4.78-4.70 (m, 1H), 4.56-4.55 (m, 1H), 3.65-3.53 (m, 5H), 3.36 (s, 3H), 3.10-3.00 (m, 2H), 2.93-2.83 (m, 2H), 2.66-2.57 (m, 6H), 2.47-2.43 (m, 1H), 2.28-2.15 (m, 1H), 2.06-1.84 (m, 7H), 1.77-1.40 (m, 4H), 1.15-1.02 (m, 6H). LC-MS (ESI): m/z [M+H]⁺=911.

### Example 262: (1S,3R)-3-(3-((2-((4R)-1-((1-(1-(2,6-dioxopiperidin-3-yl)-4-methoxy-3-methyl-2-oxo-2,3-dihydro-1H-benzo[d]imidazol-5-yl)piperidin-4-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl isopropylcarbamate

### Step 1: 1-(1-(2,6-dioxopiperidin-3-yl)-4-methoxy-3-methyl-2-oxo-2,3-dihydro-1H-benzo[d]imidazol-5-yl)piperidine-4-carbaldehyde

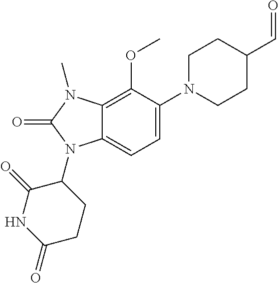 [SMILES: COc1c(N2CCC(C=O)CC2)ccc2c1n(C)c(=O)n2C1CCC(=O)NC1=O]

3-(5-(4-(dimethoxymethyl)piperidin-1-yl)-4-methoxy-3-methyl-2-oxo-2,3-dihydro-1H-benzo[d]imidazol-1-yl)piperidine-2,6-dione (50.0 mg, 0.11 mmol) was dissolved in formic acid (3 mL), then the mixture was stirred for overnight at rt. The reaction was detected by LCMS, after completion the solution was concentrated under reduced pressure and used for the next step directly. (44.0 mg, quantitative yield). LC-MS (ESI): m/z [M+H]⁺=401.4.

### Step 2: (1S,3R)-3-(3-((2-((4R)-1-((1-(1-(2,6-dioxopiperidin-3-yl)-4-methoxy-3-methyl-2-oxo-2,3-dihydro-1H-benzo[d]imidazol-5-yl)piperidin-4-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl isopropylcarbamate

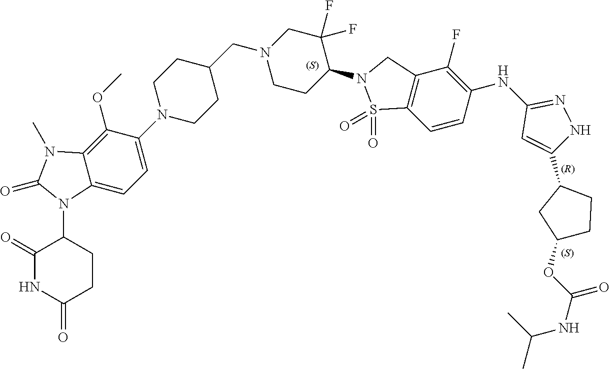

To a solution of 1-(1-(2,6-dioxopiperidin-3-yl)-4-methoxy-3-methyl-2-oxo-2,3-dihydro-1H-benzo[d]imidazol-5-yl)piperidine-4-carbaldehyde (44.0 mg, 0.11 mmol) in DMA (3 mL), (1S,3R)-3-(3-((2-((R)-3,3-difluoropiperidin-4-yl)-4-fluoro-1,1-dioxido-2,3-dihydrobenzo[d]isothiazol-5-yl)amino)-1H-pyrazol-5-yl)cyclopentyl isopropylcarbamate (65.76 mg, 0.12 mmol) were added at rt. The resulting mixture was stirred for 10 mins. Then sodium triacetoxyborohydride (70.0 mg, 0.33 mmol) was added into the mixture and stirred for 2 h. The reaction was detected by LCMS, after the residue was purified by silica gel chromatography, eluting with DCM/MeOH (10:1; R_{f}=0.31) to afford the crude product, then the crude product was purified by Prep-HPLC (Column: Xselect C18, 19*150 mm, 5 μm; Mobile Phase A: H₂O (0.1% TFA), Mobile Phase B: ACN; Flow Rate: 17 mL/min; Room Temperature; Detector: UV 214 & 254 nm; Gradient: 23% to 47% B in 11 min, 47% to 90% B in 0.2 min, 90% B hold 2 min, 90% to 23% B in 0.3 min, 23% B hold 1.5 min. to give the product (64.54 mg, 62% yield).

¹H NMR (500 MHz, DMSO-d₆) δ 11.09 (s, 1H), 8.84 (s, 1H), 8.32 (t, J=7.8 Hz, 1H), 7.61 (d, J=8.7 Hz, 1H), 7.05-6.74 (m, 3H), 5.85 (s, 1H), 5.44-5.19 (m, 1H), 5.00 (s, 1H), 4.77 (m, 1H), 4.58 (m, 1H), 4.23 (m, 8H), 3.92 (s, 3H), 3.58 (d, J=6.8 Hz, 1H), 3.50 (s, 3H), 3.36 (m, 2H), 3.01 (m, 1H), 2.89 (m, 1H), 2.63 (m, 6H), 1.99 (m, 3H), 1.86 (m, 3H), 1.72 (m, 2H), 1.59 (m, 1H), 1.42 (m, 2H), 1.02 (m, 6H). LC-MS (ESI): m/z [M+H]⁺=941.58.

### Example 263: (1S,3R)-3-(3-((2-((4R)-1′-(1-(2,6-dioxopiperidin-3-yl)-4-methoxy-3-methyl-2-oxo-2,3-dihydro-1H-benzo[d]imidazol-5-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

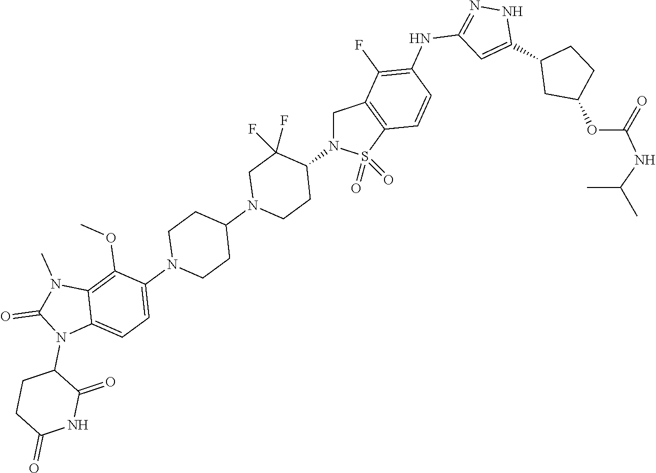 [SMILES: COc1c(N2CCC(N3CC[C@@H](N4Cc5c(ccc(Nc6cc([C@@H]7CC[C@H](OC(=O)NC(C)C)C7)[nH]n6)c5F)S4(=O)=O)C(F)(F)C3)CC2)ccc2c1n(C)c(=O)n2C1CCC(=O)NC1=O]

The titled compound was synthesized in the procedures similar to Example 105. ¹H NMR (500 MHz, DMSO-d₆) δ 12.01 (s, 1H), 11.07 (s, 1H), 8.82 (s, 1H), 8.38-8.29 (m, 1H), 7.58 (d, J=10 Hz, 1H), 6.97-6.91 (m, 1H), 6.85-6.78 (m, 1H), 6.66-6.58 (m, 1H), 5.84 (s, 1H), 5.33-5.26 (m, 1H), 5.05-4.96 (m, 1H), 4.81-4.70 (m, 1H), 4.58-4.50 (m, 1H), 4.04-3.88 (m, 4H), 3.63-3.53 (m, 1H), 3.49 (s, 3H), 3.44-3.36 (m, 2H), 3.27-3.20 (m, 1H), 3.112-2.99 (m, 2H), 2.94-2.83 (m, 1H), 2.82-2.58 (m, 6H), 2.48-2.45 (m, 1H), 2.21-2.10 (m, 1H), 2.08-1.81 (m, 7H), 1.79-1.55 (m, 5H), 1.07-0.99 (m, 6H). LC-MS (ESI): m/z [M+H]⁺=927.

### Example 264 and 265: (1S,3R)-3-(3-((2-((R)-1-((1-(1-((S)-2,6-dioxopiperidin-3-yl)-7-fluoro-3,4-dimethyl-2-oxo-2,3-dihydro-1H-benzo[d]imidazol-5-yl)piperidin-4-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-((R)-1-((1-(1-((R)-2,6-dioxopiperidin-3-yl)-7-fluoro-3,4-dimethyl-2-oxo-2,3-dihydro-1H-benzo[d]imidazol-5-yl)piperidin-4-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl isopropylcarbamate

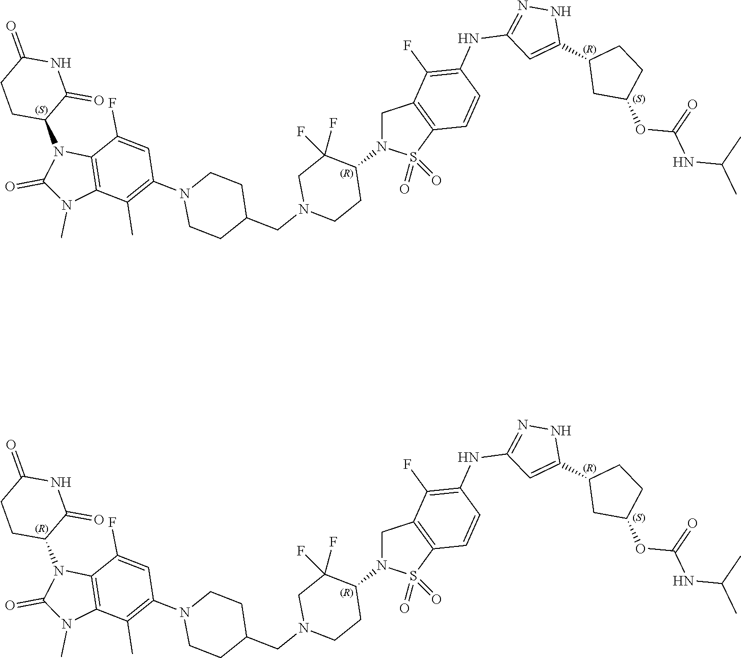

The titled compound was synthesized in the procedures similar to Example 183 in a racemate form, which was further separated by chiral HPLC to give:

Enantiomer 1 (Example 264, 100% ee); Retention time: 5.175 min. ¹H NMR (500 MHz, DMSO) δ 12.01 (s, 1H), 11.08 (s, 1H), 8.82 (s, 1H), 8.33 (t, J=8.0 Hz, 1H), 7.58 (d, J=8.7 Hz, 1H), 6.94 (d, J=7.4 Hz, 1H), 6.66 (d, J=13.1 Hz, 1H), 5.84 (s, 1H), 5.47 (d, J=7.6 Hz, 1H), 5.00 (s, 1H), 4.75 (d, J=14.3 Hz, 1H), 4.54 (d, J=13.4 Hz, 1H), 4.10-3.92 (m, 1H), 3.66-3.53 (m, 4H), 3.17 (t, J=10.4 Hz, 1H), 3.12-2.90 (m, 5H), 2.67-2.54 (m, 6H), 2.39-2.30 (m, 3H), 2.27-2.15 (m, 2H), 2.10-1.99 (m, 2H), 1.95-1.86 (m, 1H), 1.84-1.59 (m, 7H), 1.35-1.25 (m, 2H), 1.07-0.98 (m, 6H). LC-MS (ESI): m/z [M+H]⁺=943.67.

Enantiomer 2 (Example 265, 100% ee); Retention time: 7.211 min. ¹H NMR (500 MHz, DMSO) δ 12.01 (s, 1H), 11.08 (s, 1H), 8.82 (s, 1H), 8.33 (t, J=8.0 Hz, 1H), 7.58 (d, J=8.7 Hz, 1H), 6.94 (d, J=7.4 Hz, 1H), 6.66 (d, J=13.1 Hz, 1H), 5.84 (s, 1H), 5.47 (d, J=7.6 Hz, 1H), 5.00 (s, 1H), 4.75 (d, J=14.3 Hz, 1H), 4.54 (d, J=13.4 Hz, 1H), 4.10-3.92 (m, 1H), 3.66-3.53 (m, 4H), 3.17 (t, J=10.4 Hz, 1H), 3.12-2.90 (m, 5H), 2.67-2.54 (m, 6H), 2.39-2.30 (m, 3H), 2.27-2.15 (m, 2H), 2.10-1.99 (m, 2H), 1.95-1.86 (m, 1H), 1.84-1.59 (m, 7H), 1.35-1.25 (m, 2H), 1.07-0.98 (m, 6H). LC-MS (ESI): m/z [M+H]⁺=948.38.

Chiral analytical method: Column: YMC Amylose-SA 4.6*250 mm 5 μm Mobile phase: A for MtBE (0.1%2M NH3 MeOH) (%) and B for MeOH:DCM=50:50 (v/v); Gradient: Mobile Phase A: Mobile Phase B=20:80 (v/v); HPLC Equipment: Agilent 1260 Infinity II (ARD_LC-808); Column temperature: 25° C.

Chiral Prep-HPLC Condition: YMC Amylose-SA 4.6*250 mm 5 μm Mobile phase: A for MtBE (0.1%2M NH3 MeOH)(%) and B for MeOH:DCM=50:50; Gradient: Mobile Phase A: Mobile Phase B=20:80 (v/v); Flow Rate: 1 mL/min, Wavelength: UV 250 nm and 280 nm, Prep-HPLC Equipment: Prep-HPLC Gilson (ARD_LC-815); Column temperature: 25° C.

### Example 266: (1S,3R)-3-(3-((2-((4S)-1′-(1-(2,6-dioxopiperidin-3-yl)-4-methoxy-3-methyl-2-oxo-2,3-dihydro-1H-benzo[d]imidazol-5-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

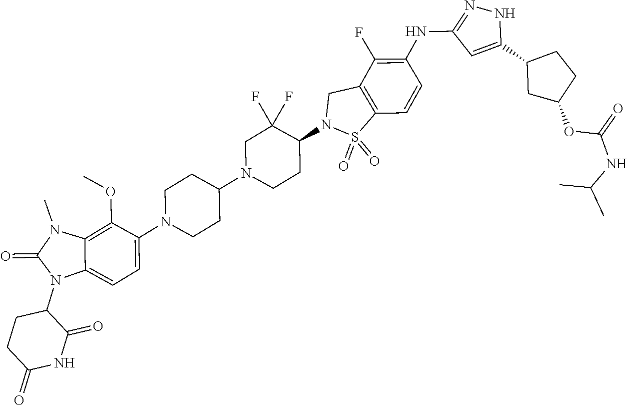 [SMILES: COc1c(N2CCC(N3CC[C@H](N4Cc5c(ccc(Nc6cc([C@@H]7CC[C@H](OC(=O)NC(C)C)C7)[nH]n6)c5F)S4(=O)=O)C(F)(F)C3)CC2)ccc2c1n(C)c(=O)n2C1CCC(=O)NC1=O]

The titled compound was synthesized in the procedures similar to Example 105. ¹H NMR (500 MHz, DMSO-d₆) δ 12.01 (s, 1H), 11.08 (s, 1H), 8.82 (s, 1H), 8.38-8.26 (m, 1H), 7.59 (d, J=10 Hz, 1H), 6.97-6.91 (m, 1H), 6.85-6.78 (m, 1H), 6.66-6.58 (m, 1H), 5.84 (s, 1H), 5.33-5.24 (m, 1H), 5.07-4.95 (m, 1H), 4.80-4.69 (m, 1H), 4.58-4.50 (m, 1H), 4.04-3.91 (m, 4H), 3.79-3.68 (m, 1H), 3.57 (s, 3H), 3.47-3.41 (m, 2H), 3.39-3.28 (m, 1H), 3.08-2.98 (m, 2H), 2.96-2.85 (m, 1H), 2.69-2.57 (m, 7H), 2.48-2.45 (m, 1H), 2.20-2.10 (m, 1H), 2.08-1.74 (m, 6H), 1.76-1.55 (m, 5H), 1.07-0.99 (m, 6H). LC-MS (ESI): m/z [M+H]⁺=927.

### Example 267 and 268: (1S,3R)-3-(3-((2-((R)-1-((1-(1-((S)-2,6-dioxopiperidin-3-yl)-8-fluoro-2-oxo-1,2,3,4-tetrahydro-5-oxa-1,2a-diazaacenaphthylen-6-yl)piperidin-4-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-((R)-1-((1-(1-((R)-2,6-dioxopiperidin-3-yl)-8-fluoro-2-oxo-1,2,3,4-tetrahydro-5-oxa-1,2a-diazaacenaphthylen-6-yl)piperidin-4-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl isopropylcarbamate

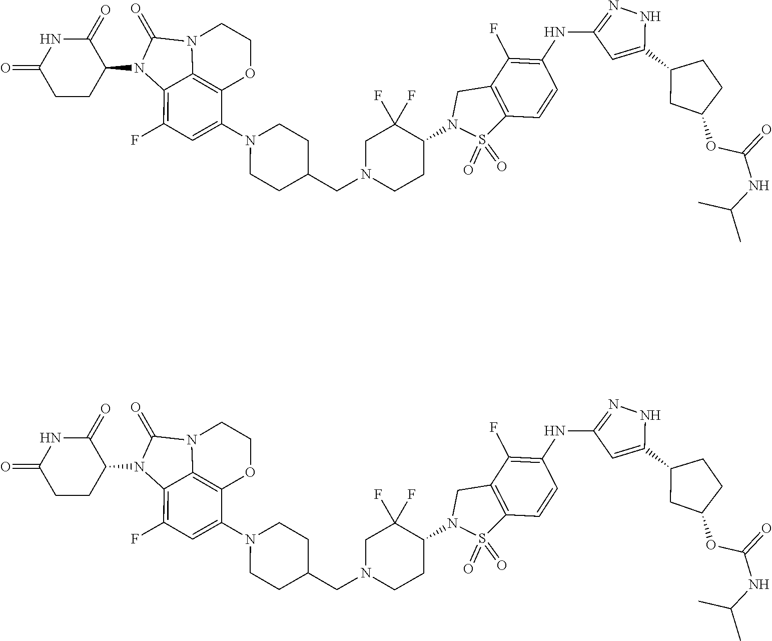 [SMILES: CC(C)NC(=O)O[C@H]1CC[C@@H](c2cc(Nc3ccc4c(c3F)CN([C@@H]3CCN(CC5CCN(c6cc(F)c7c8c6OCCn8c(=O)n7[C@H]6CCC(=O)NC6=O)CC5)CC3(F)F)S4(=O)=O)n[nH]2)C1 ; CC(C)NC(=O)O[C@H]1CC[C@@H](c2cc(Nc3ccc4c(c3F)CN([C@@H]3CCN(CC5CCN(c6cc(F)c7c8c6OCCn8c(=O)n7[C@@H]6CCC(=O)NC6=O)CC5)CC3(F)F)S4(=O)=O)n[nH]2)C1]

The titled compound was synthesized in the procedures similar to Example 109 in a racemate form, which was further separated by chiral HPLC to give:

Enantiomer 1 (Example 267, 100% ee); Retention time: 5.387 min. ¹H NMR (500 MHz, DMSO) δ 12.00 (s, 1H), 11.11 (s, 1H), 8.82 (s, 1H), 8.33 (t, J=8.0 Hz, 1H), 7.58 (d, J=8.7 Hz, 1H), 6.94 (d, J=7.2 Hz, 1H), 6.40 (d, J=13.4 Hz, 1H), 5.84 (s, 1H), 5.39 (s, 1H), 5.06-4.96 (m, 1H), 4.75 (d, J=14.4 Hz, 1H), 4.54 (d, J=13.3 Hz, 1H), 4.39-4.33 (m, 2H), 4.07-3.86 (m, 3H), 3.63-3.54 (m, 1H), 3.43-3.37 (m, 2H), 3.21-3.12 (m, 1H), 3.11-2.92 (m, 3H), 2.62-2.54 (m, 7H), 2.46-2.42 (m, 1H), 2.35-2.31 (m, 3H), 2.25-2.15 (m, 2H), 2.13-2.08 (m, 1H), 2.05-1.98 (m, 1H), 1.91 (dd, J=13.2, 5.3 Hz, 1H), 1.84-1.68 (m, 5H), 1.64-1.54 (m, 2H), 1.32-1.22 (m, 2H), 1.03 (d, J=6.4 Hz, 6H). LC-MS (ESI): m/z [M+H]⁺=957.4;

Enantiomer 2 (Example 268, 100% ee); Retention time: 8.816 min. ¹H NMR (500 MHz, DMSO) δ 12.00 (s, 1H), 11.11 (s, 1H), 8.82 (s, 1H), 8.33 (t, J=8.0 Hz, 1H), 7.58 (d, J=8.7 Hz, 1H), 6.94 (d, J=7.2 Hz, 1H), 6.40 (d, J=13.4 Hz, 1H), 5.84 (s, 1H), 5.39 (s, 1H), 5.06-4.96 (m, 1H), 4.75 (d, J=14.4 Hz, 1H), 4.54 (d, J=13.3 Hz, 1H), 4.39-4.33 (m, 2H), 4.07-3.86 (m, 3H), 3.63-3.54 (m, 1H), 3.43-3.37 (m, 2H), 3.21-3.12 (m, 1H), 3.11-2.92 (m, 3H), 2.62-2.54 (m, 7H), 2.46-2.42 (m, 1H), 2.35-2.31 (m, 3H), 2.25-2.15 (m, 2H), 2.13-2.08 (m, 1H), 2.05-1.98 (m, 1H), 1.91 (dd, J=13.2, 5.3 Hz, 1H), 1.84-1.68 (m, 5H), 1.64-1.54 (m, 2H), 1.32-1.22 (m, 2H), 1.03 (d, J=6.4 Hz, 6H). LC-MS (ESI): m/z [M+H]⁺=957.4;

Chiral analytical method: Column: ICHIRALPAK IC-5 4.6*150 mm 5 μm; Mobile phase: A for MtBE (0.1%2M NH3 MeOH) (%) and B for MeOH:DCM=50:50(%); Gradient: Mobile Phase A: Mobile Phase B=30:70 (v/v); HPLC Equipment: HPLC-Agilent, Back pressure: 100 bar; Column temperature: 35° C.

Chiral Prep-HPLC Condition: CHIRALPAK IC-5 250*20.0 mml, 5 μm; Mobile phase: A for MtBE (0.1%2M NH3 MeOH) (%) and B for MeOH:DCM=50:50; Gradient: Mobile Phase A: Mobile Phase B=30:70 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 269 and 270: (1S,3R)-3-(3-((2-((R)-1′-(1-((R)-2,6-dioxopiperidin-3-yl)-8-fluoro-2-oxo-1,2,3,4-tetrahydro-5-oxa-1,2a-diazaacenaphthylen-6-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-((R)-1′-(1-((S)-2,6-dioxopiperidin-3-yl)-8-fluoro-2-oxo-1,2,3,4-tetrahydro-5-oxa-1,2a-diazaacenaphthylen-6-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

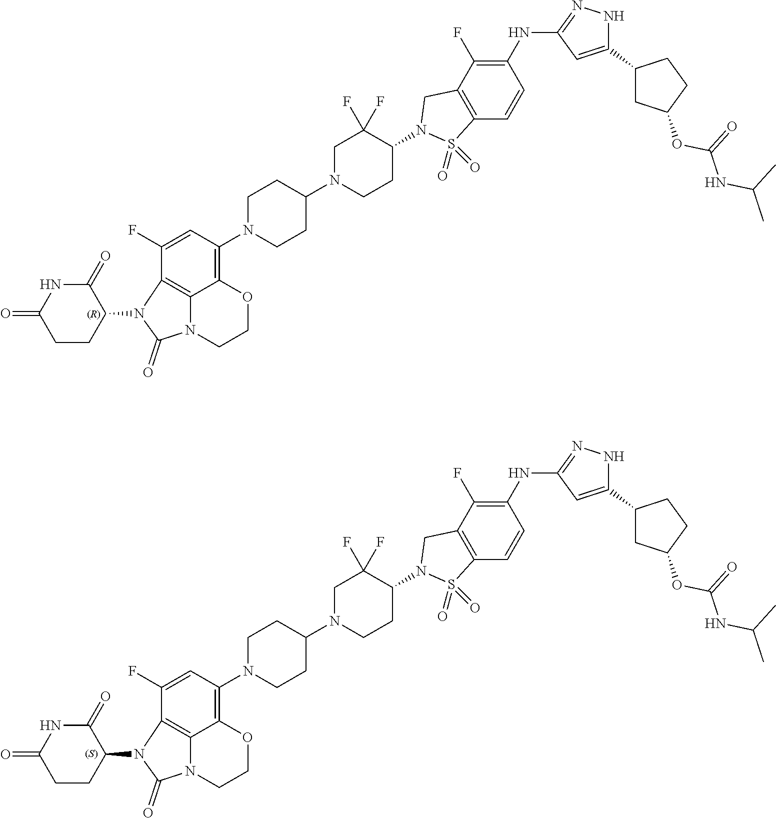

The racemic compound was synthesized in the procedures similar to Example 187, which was further separated by Chiral Prep-HPLC to afford the desired product:

Enantiomer 1 (Example 270, 100% ee); Retention time: 6.74 min. ¹H NMR (500 MHz, DMSO-d₆) δ 12.01 (s, 1H), 11.11 (s, 1H), 8.82 (s, 1H), 8.32 (t, J=5 Hz, 1H), 7.58 (d, J=10 Hz, 1H), 6.96-6.92 (m, 1H), 6.41 (d, J=10 Hz, 1H), 5.84 (s, 1H), 5.45-5.26 (m, 1H), 5.03-4.96 (m, 1H), 4.78-4.70 (m, 1H), 4.57-4.50 (m, 1H), 4.41-4.34 (m, 2H), 4.02-3.89 (m, 3H), 3.63-3.54 (m, 1H), 3.50-3.42 (m, 2H), 3.25-3.18 (m, 1H), 3.12-2.93 (m, 3H), 2.82-2.70 (m, 1H), 2.67-2.57 (m, 5H), 2.44-2.42 (m, 2H), 2.18-2.06 (m, 2H), 2.06-1.98 (m, 1H), 1.96-1.88 (m, 1H), 1.87-1.66 (m, 5H), 1.66-1.54 (m, 3H), 1.09-0.97 (m, 6H). LC-MS (ESI): m/z [M+H]⁺=943.

Enantiomer 2 (Example 269, 100% ee); Retention time: 9.7 min. ¹H NMR (500 MHz, DMSO-d₆) δ 12.01 (s, 1H), 11.11 (s, 1H), 8.82 (s, 1H), 8.32 (t, J=5 Hz, 1H), 7.58 (d, J=10 Hz, 1H), 6.96-6.92 (m, 1H), 6.41 (d, J=10 Hz, 1H), 5.84 (s, 1H), 5.45-5.26 (m, 1H), 5.03-4.96 (m, 1H), 4.78-4.70 (m, 1H), 4.57-4.50 (m, 1H), 4.41-4.34 (m, 2H), 4.02-3.89 (m, 3H), 3.63-3.54 (m, 1H), 3.50-3.42 (m, 2H), 3.25-3.18 (m, 1H), 3.12-2.93 (m, 3H), 2.82-2.70 (m, 1H), 2.67-2.57 (m, 5H), 2.44-2.42 (m, 2H), 2.18-2.06 (m, 2H), 2.06-1.98 (m, 1H), 1.96-1.88 (m, 1H), 1.87-1.66 (m, 5H), 1.66-1.54 (m, 3H), 1.09-0.97 (m, 6H). LC-MS (ESI): m/z [M+H]⁺=943.

Chiral analytical method: Amylose-SA, 4.6 mm×250 mm, 5 um; Mobile phase: A for MtBE (0.1% 2M NH3 MeOH) and B for DCM:MeOH (50:50); Gradient: Mobile Phase A: Mobile Phase B=40:60 (v/v); HPLC Equipment: HPLC-Agilent, Back pressure: 100 bar; Column temperature: 35° C.

Chiral Prep-HPLC Condition: Column: Cellulose-SA, 250 mm×20 mm, 5 um; Mobile phase: A for MtBE (0.1% 2M NH3 MeOH) and B for DCM:MeOH (50:50); Gradient: Mobile Phase A: Mobile Phase B=40:60 (v/v); Flow Rate: 20 mL/min, Wave Length: UV 200 nm and 270 nm, Prep-HPLC Equipment: Prep-HPLC-Gilson, Back pressure: 100 bar; Column temperature: 25° C.

### Example 271: (1S,3R)-3-(3-((2-(1-((1-(3-(2,6-dioxopiperidin-3-yl)-2-oxo-2,3-dihydrobenzo[d]oxazol-6-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

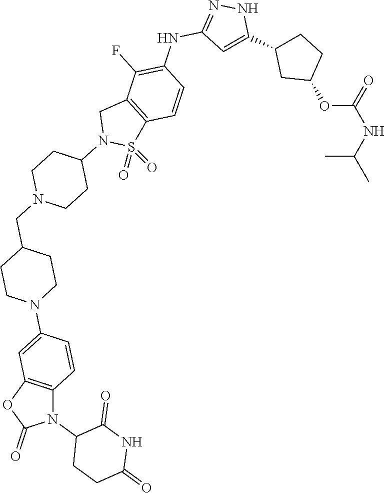 [SMILES: CC(C)NC(=O)O[C@H]1CC[C@@H](c2cc(Nc3ccc4c(c3F)CN(C3CCN(CC5CCN(c6ccc7c(c6)oc(=O)n7C6CCC(=O)NC6=O)CC5)CC3)S4(=O)=O)n[nH]2)C1]

The titled compound was synthesized in the procedures similar to Example 62. ¹H NMR (500 MHz, DMSO-d₆) δ 11.99 (s, 1H), 11.17 (s, 1H), 8.76 (s, 1H), 8.28 (s, 1H), 7.51 (d, J=5 Hz, 1H), 7.12-7.03 (m, 2H), 6.98-6.91 (m, 1H), 6.81-6.75 (m, 1H), 5.83 (s, 1H), 5.33-5.24 (m, 1H), 5.06-4.96 (m, 1H), 4.52 (s, 2H), 3.65-3.52 (m, 3H), 3.48-3.39 (m, 1H), 3.11-3.01 (m, 1H), 2.95-2.82 (m, 3H), 2.74-2.58 (m, 4H), 2.47-2.43 (m, 1H), 2.23-2.08 (m, 3H), 2.07-1.96 (m, 3H), 1.95-1.82 (m, 5H), 1.81-1.55 (m, 6H), 1.28-1.14 (m, 2H), 1.08-0.95 (m, 6H). LC-MS (ESI): m/z [M+H]⁺=862.

### Example 272: (1S,3R)-3-(3-((2-(1′-(3-(2,6-dioxopiperidin-3-yl)-2-oxo-2,3-dihydrobenzo[d]oxazol-6-yl)-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

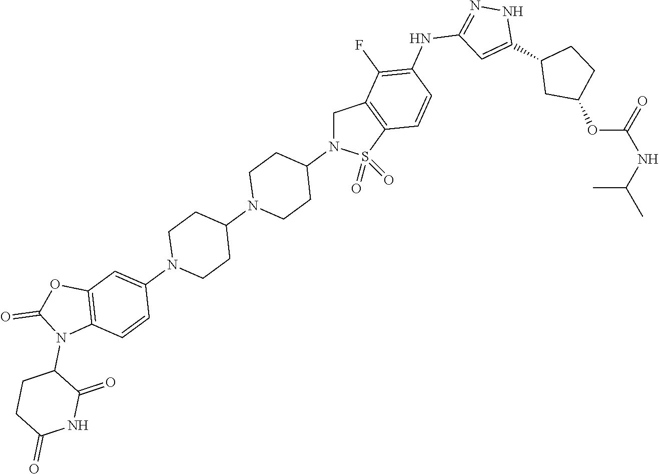 [SMILES: CC(C)NC(=O)O[C@H]1CC[C@@H](c2cc(Nc3ccc4c(c3F)CN(C3CCN(C5CCN(c6ccc7c(c6)oc(=O)n7C6CCC(=O)NC6=O)CC5)CC3)S4(=O)=O)n[nH]2)C1]

The titled compound was synthesized in the procedures similar to Example 63. ¹H NMR (500 MHz, DMSO-d₆) δ 11.99 (s, 1H), 11.17 (s, 1H), 8.76 (s, 1H), 8.29 (s, 1H), 7.50 (d, J=10 Hz, 1H), 7.15-7.06 (s, 2H), 6.85-6.76 (m, 2H), 5.83 (s, 1H), 5.46-5.28 (m, 1H), 5.11-4.96 (m, 1H), 4.51 (s, 2H), 3.68-2.60 (m, 2H), 3.55-3.52 (m, 1H), 3.09-3.01 (m, 1H), 3.01-2.94 (m, 2H), 2.98-2.82 (m, 1H), 2.72-2.61 (m, 6H), 2.42-2.33 (m, 3H), 2.17-2.08 (m, 1H), 2.06-1.98 (m, 1H), 1.97-1.87 (m, 3H), 1.87-1.77 (m, 4H), 1.76-1.65 (m, 2H), 1.63-1.50 (m, 3H), 1.11-0.95 (m, 6H). LC-MS (ESI): m/z [M+H]⁺=848.

### Example 273: (1S,3R)-3-(3-((2-(1-((1-(3-(2,6-dioxopiperidin-3-yl)-7-fluoro-2-oxo-2,3-dihydrobenzo[d]oxazol-6-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

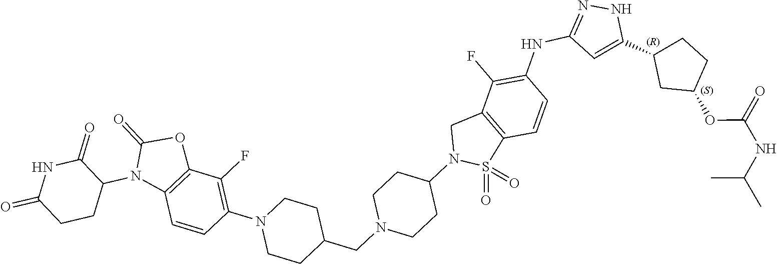

### Step 1: 3-(2,6-bis(benzyloxy)pyridin-3-yl)-6-bromo-7-fluorobenzo[d]oxazol-2(3H)-one

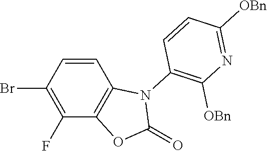 [SMILES: O=c1oc2c(F)c(Br)ccc2n1-c1ccc(OCc2ccccc2)nc1OCc1ccccc1]

To a mixture 6-bromo-7-fluorobenzo[d]oxazol-2(3H)-one (2 g, 8.66 mmol) and 2,6-bis(benzyloxy)-3-(4,4,5,5-tetramethyl-1,3,2-dioxaborolan-2-yl)pyridine (7.21 g, 17.3 mmol) in andydrous 1,4-dioxane (10 mL) was added Cu(OAc)₂ (3.15 mg, 17.3 mmol) and Py (1.38 g, 17.3 mmol). The reaction mixture was stirred under nitrogen atmosphere at 100° C. for 16 h. The mixture was filtered, and the filtrate was concentrated under reduced pressure. The residue was purified by silica gel column chromatography, eluting with PE/EA (2:1) to afford the product (1 g, 11% yield). LC-MS (ESI): m/z [M+H]⁺=521.2.

### Step 2: (1S,3R)-3-(3-((2-(1-((1-(3-(2,6-dioxopiperidin-3-yl)-7-fluoro-2-oxo-2,3-dihydrobenzo[d]oxazol-6-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

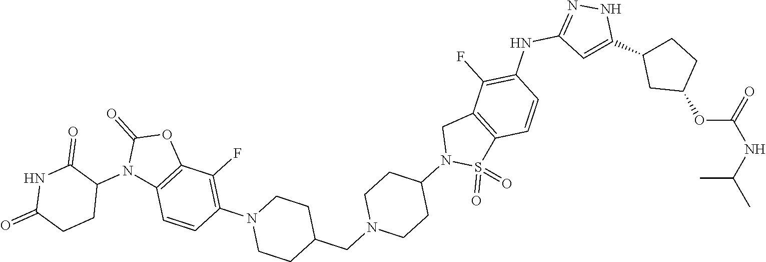 [SMILES: CC(C)NC(=O)O[C@H]1CC[C@@H](c2cc(Nc3ccc4c(c3F)CN(C3CCN(CC5CCN(c6ccc7c(oc(=O)n7C7CCC(=O)NC7=O)c6F)CC5)CC3)S4(=O)=O)n[nH]2)C1]

The titled compound was synthesized in the procedures similar to Example 37. ¹H NMR (500 MHz, DMSO) δ 12.00 (s, 1H), 11.22 (s, 1H), 8.77 (s, 1H), 8.28 (s, 1H), 7.51 (d, J=8.6 Hz, 1H), 7.00 (d, J=8.6 Hz, 1H), 6.96-6.86 (m, 2H), 5.83 (s, 1H), 5.35 (dd, J=13.0, 5.3 Hz, 1H), 5.00 (s, 1H), 4.52 (s, 2H), 3.58 (q, J=13.2, 6.5 Hz, 1H), 3.49-3.38 (m, 2H), 3.26 (d, J=11.5 Hz, 2H), 3.10-3.01 (m, 1H), 2.94-2.82 (m, 3H), 2.71-2.61 (m, 4H), 2.23-2.12 (m, 3H), 2.09-1.98 (m, 3H), 1.96-1.79 (m, 7H), 1.77-1.55 (m, 4H), 1.29 (q, J=21.3, 11.3 Hz, 2H), 1.08-0.97 (m, 6H). LC-MS (ESI): m/z [M+H]⁺=880.54.

### Example 274: (1S,3R)-3-(3-((2-(1-((1-(3-(2,6-dioxopiperidin-3-yl)-5-fluoro-2-oxo-2,3-dihydrobenzo[d]oxazol-6-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropyl carbamate

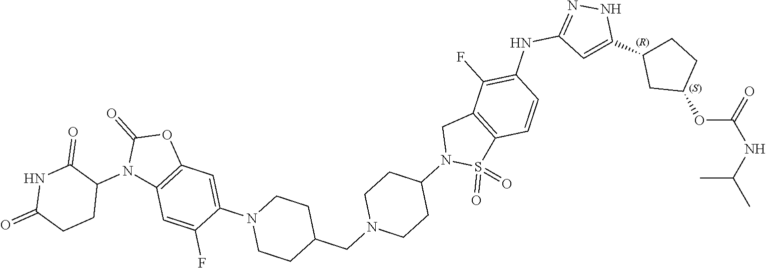

### Step 1: 1-(3-(2,6-dioxopiperidin-3-yl)-5-fluoro-2-oxo-2,3-dihydrobenzo[d]oxazol-6-yl)piperidine-4-carbaldehyde

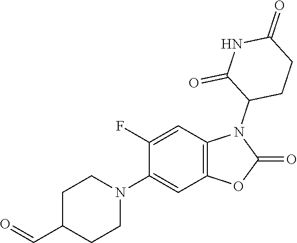 [SMILES: O=CC1CCN(c2cc3oc(=O)n(C4CCC(=O)NC4=O)c3cc2F)CC1]

The titled compound was synthesized in the procedures similar to Example 62. LC-MS (ESI): m/z [M+H]⁺=376.4

### Step 2: (1S,3R)-3-(3-((2-(1-((1-(3-(2,6-dioxopiperidin-3-yl)-5-fluoro-2-oxo-2,3-dihydrobenzo[d]oxazol-6-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropyl carbamate

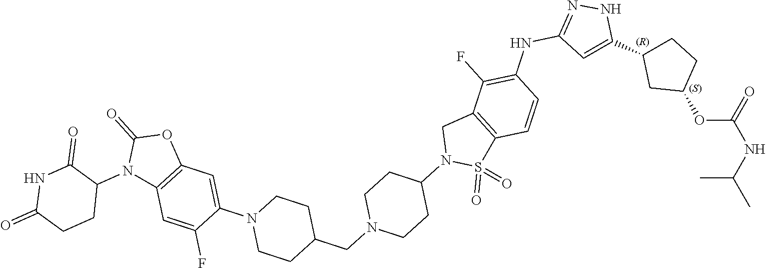

The titled compound was synthesized in the procedures similar to Example 62. ¹H NMR (500 MHz, DMSO) δ 11.99 (s, 1H), 11.18 (s, 1H), 8.76 (s, 1H), 8.29 (s, 1H), 8.17 (s, 1H), 7.51 (d, J=8.7 Hz, 1H), 7.30 (d, J=11.5 Hz, 1H), 7.20 (d, J=7.2 Hz, 1H), 6.94 (d, J=7.4 Hz, 1H), 5.83 (s, 1H), 5.31 (q, J=13.0, 5.3 Hz, 1H), 4.99 (s, 1H), 4.52 (s, 2H), 3.63-3.53 (m, 1H), 3.49-3.39 (m, 1H), 3.23 (d, J=11.4 Hz, 2H), 3.11-3.01 (m, 1H), 2.90 (d, J=10.9 Hz, 2H), 2.66-2.61 (m, 4H), 2.39-2.34 (m, 2H), 2.20 (d, J=6.9 Hz, 2H), 2.15-2.10 (m, 1H), 2.20-1.97 (m, 3H), 1.93-1.57 (m, 11H), 1.27 (q, J=21.5, 12.0 Hz, 2H), 1.07-0.97 (m, 6H). LC-MS (ESI): m/z [M+H]⁺=880.60.

### Example 275: (1S,3R)-3-(3-((2-(1-((1-(3-((R)-2,6-dioxopiperidin-3-yl)-4-methyl-2-oxo-2,3-dihydrobenzo[d]oxazol-6-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

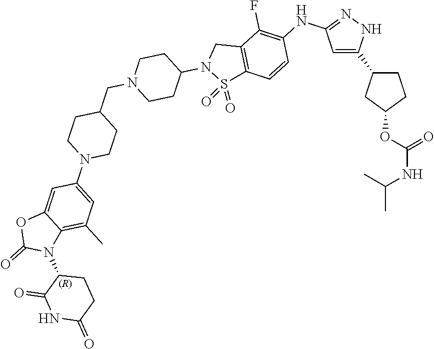

### Step 1: 3-(2,6-bis(benzyloxy)pyridin-3-yl)-6-(4-(dimethoxymethyl)piperidin-d-yl)-4-methylbenzo[d]oxazol-2(3H)-one

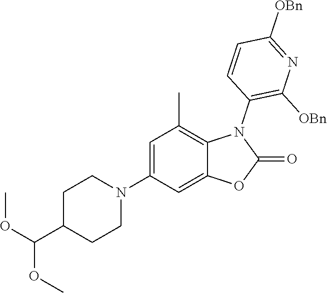 [SMILES: COC(OC)C1CCN(c2cc(C)c3c(c2)oc(=O)n3-c2ccc(OCc3ccccc3)nc2OCc2ccccc2)CC1]

Into a 250-mL round-bottom flask purged and maintained with N₂, were added 3-(2,6-bis(benzyloxy)pyridin-3-yl)-6-bromo-4-methylbenzo[d]oxazol-2 (3H)-one (1.20 g, 2.33 mmol), 4-(dimethoxymethyl)piperidine (0.55 g, 3.49 mmol), Cs₂CO₃ (2.27 g, 6.99 mmol), Ruphos (217.6 mg, 0.46 mmol), Pd₂(dba)₃ (210.5 mg, 0.23 mmol) and Dioxane (40 mL) at room temperature. The resulting mixture was stirred for 2 h at 100° C. The resulting mixture was concentrated under vacuum. The residue was purified by silica gel column chromatography, eluted with PE/EtOAc (100:0-70:30) to afford 3-(2,6-bis(benzyloxy)pyridin-3-yl)-6-(4-(dimethoxymethyl)piperidin-1-yl)-4-methylbenzo[d]oxazol-2(3H)-one (460.0 mg, 33.24%) as yellow solid.

### Step 2: 3-(6-(4-(dimethoxymethyl)piperidin-1-yl)-4-methyl-2-oxobenzo[d]oxazol-3(2H)-yl)piperidine-2,6-dione

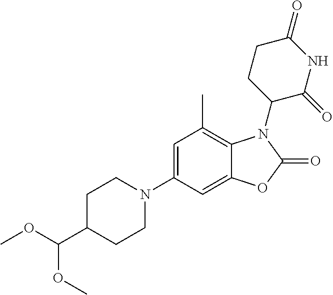 [SMILES: COC(OC)C1CCN(c2cc(C)c3c(c2)oc(=O)n3C2CCC(=O)NC2=O)CC1]

Into a 250-mL round-bottom flask purged and maintained with H₂, were added 3-(2,6-bis(benzyloxy)pyridin-3-yl)-6-(4-(dimethoxymethyl)piperidin-1-yl)-4-methylbenzo[d]oxazol-2(3H)-one (460.0 mg, 0.77 mmol), Pd/C (460.0 mg), AcOH (50.0 mg) and THF (30 mL) at room temperature. The resulting mixture was stirred for 15 h at 50° C. The precipitated solids were collected by filtration and washed with THF (300 mL). The residue was purified by silica gel column chromatography, eluted with PE/EtOAc (100:0-50:50) to afford 3-(6-(4-(dimethoxymethyl)piperidin-1-yl)-4-methyl-2-oxobenzo[d]oxazol-3(2H)-yl)piperidine-2,6-dione (71.3 mg, 22.12%) as light blue solid.

### Step 3:1-(3-(2,6-dioxopiperidin-3-yl)-4-methyl-2-oxo-2,3-dihydrobenzo[d]oxazol-6-yl)piperidine-4-carbaldehyde

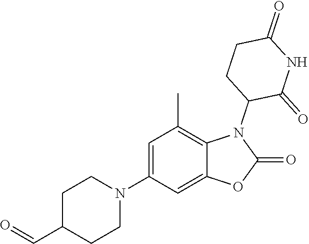 [SMILES: Cc1cc(N2CCC(C=O)CC2)cc2oc(=O)n(C3CCC(=O)NC3=O)c12]

To a flask was added 3-(6-(4-(dimethoxymethyl)piperidin-1-yl)-4-methyl-2-oxobenzo[d]oxazol-3(2H)-yl)piperidine-2,6-dione (50 mg, 0.12 mmol) and formic acid (5 mL). The mixture was stirred at room temperature for 2 h. The solution was concentrated under reduced pressure to give the crude product. LC-MS (ESI): m/z [M+H]⁺=318.2

### Step 4: (1S,3R)-3-(3-((2-(1-((1-(3-((R)-2,6-dioxopiperidin-3-yl)-4-methyl-2-oxo-2,3-dihydrobenzo[d]oxazol-6-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

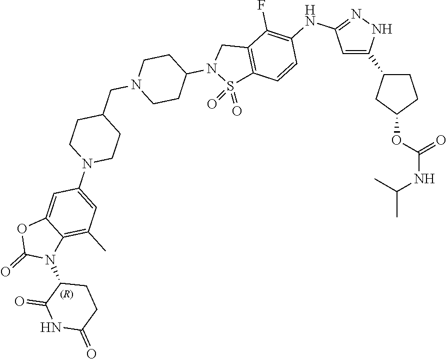

The racemic compound was synthesized in the procedures similar to Example 1, which was further separated by Chiral Prep-HPLC to afford the desired product:

Enantiomer 2 (Example 1b, 100% ee); Retention time: 9.38 min. ¹H NMR (500 MHz, DMSO-d₆) δ 12.00 (s, 1H), 11.13 (s, 1H), 8.77 (s, 1H), 8.31 (t, J=5 Hz, 1H), 7.53 (t, J=10 Hz, 1H), 6.97-6.87 (m, 2H), 6.65-6.55 (m, 1H), 5.83 (s, 1H), 5.50-5.42 (m, 1H), 5.31-5.23 (m, 1H), 5.04-4.96 (m, 1H), 4.53 (s, 2H), 3.71-3.52 (m, 4H), 3.49-3.40 (m, 1H), 3.15-2.98 (m, 3H), 2.94-2.81 (m, 2H), 2.68-2.59 (m, 4H), 2.45 (s, 3H), 2.30-2.11 (m, 4H), 2.07-1.98 (m, 2H), 1.96-1.83 (m, 2H), 1.82-1.55 (m, 9H), 1.39-1.14 (m, 3H), 1.09-0.95 (m, 6H). LC-MS (ESI): m/z [M+H]⁺=890.

Chiral analytical method: i-Cellulose-5, 4.6 mm×250 mm, 5 um; Mobile phase: A for MtBE (0.1% 2M NH3 MeOH) and B for DCM:MeOH (50:50); Gradient: Mobile Phase A: Mobile Phase B=30:70 (v/v); HPLC Equipment: HPLC-Agilent, Back pressure: 100 bar; Column temperature: 35° C.

Chiral Prep-HPLC Condition: Column: i-Cellulose-5, 250 mm×20 mm, 5 um; Mobile phase: A for MtBE (0.1% 2M NH3 MeOH) and B for DCM:MeOH (50:50); Gradient: Mobile Phase A: Mobile Phase B=30:70 (v/v); Flow Rate: 20 mL/min, Wave Length: UV 200 nm and 270 nm, Prep-HPLC Equipment: Prep-HPLC-Gilson, Back pressure: 100 bar; Column temperature: 25° C.

### Example 276: (1S,3R)-3-(3-((2-(1′-(3-((R)-2,6-dioxopiperidin-3-yl)-4-methyl-2-oxo-2,3-dihydrobenzo[d]oxazol-6-yl)-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

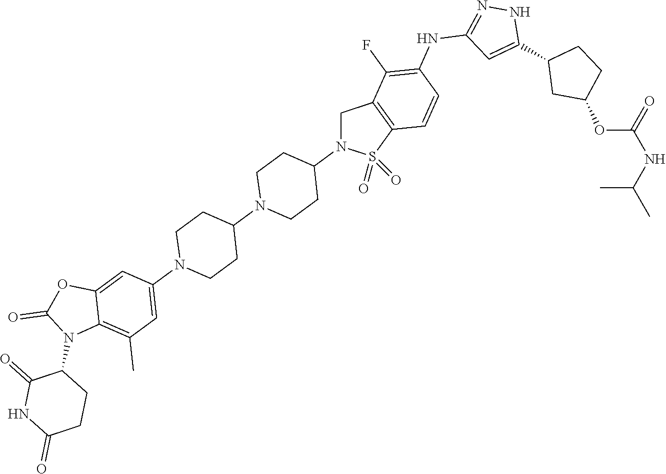 [SMILES: Cc1cc(N2CCC(N3CCC(N4Cc5c(ccc(Nc6cc([C@@H]7CC[C@H](OC(=O)NC(C)C)C7)[nH]n6)c5F)S4(=O)=O)CC3)CC2)cc2oc(=O)n([C@@H]3CCC(=O)NC3=O)c12]

### Step 1: 4-methylbenzo[d]oxazol-2(3H)-one

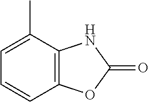

Into a 500-mL round-bottom flask purged and maintained with N₂, were added 2-amino-3-methylphenol (15.00 g, 121.95 mmol), CDI (29.63 g, 182.93 mmol) and THF (200 mL) at room temperature. The reaction was stirred for 2 h at 70° C. The resulting mixture was concentrated under vacuum. The residue was purified by silica gel column chromatography, eluted with PE/EtOAc (100:0-70:30) to afford 4-methylbenzo[d]oxazol-2(3H)-one (14.00 g, 77.05%) as yellow solid.

### Step 2: -bromo-4-methylbenzo[d]oxazol-2(3H)-one

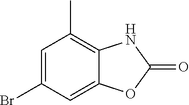

Into a 500-mL round-bottom flask purged and maintained with an inert atmosphere of nitrogen, were added 4-methylbenzo[d]oxazol-2(3H)-one (11.00 g, 73.83 mmol), NBS (14.46 g, 81.21 mmol) and MeCN (100 mL) at room temperature. The reaction was stirred for 2 h at 70° C. The resulting mixture was extracted with EtOAc (400 mL). The combined organic layers were washed with brine (2×300 mL), dried over anhydrous Na₂SO₄. The residue was purified by silica gel column chromatography, eluted with PE/EtOAc (100:0-80:20) to afford 6-bromo-4-methylbenzo[d]oxazol-2(3H)-one (12.00 g, 71.61%) as yellow solid.

### Step 3: 3-(2,6-bis(benzyloxy)pyridin-3-yl)-6-bromo-4-methylbenzo[d]oxazol-2(3H)-one

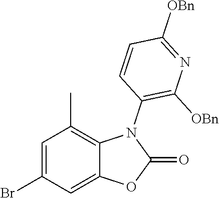 [SMILES: Cc1cc(Br)cc2oc(=O)n(-c3ccc(OCc4ccccc4)nc3OCc3ccccc3)c12]

Into a 1000-mL round-bottom flask purged and maintained with 02, were added 6-bromobenzo[d]oxazol-2(3H)-one (20.00 g, 93.90 mmol), 2,6-bis(benzyloxy)-3-(4,4,5,5-tetramethyl-1,3,2-dioxaborolan-2-yl)pyridine (58.73 g, 140.85 mmol), Cu(OAc)₂ (18.78 g, 93.90 mmol), Py (22.25 g, 281.70 mmol), 4A MS (10.00 g) and Dioxane (500 mL) at room temperature. The reaction was stirred for 15 h at 80° C. The residue was purified by silica gel column chromatography, eluted with PE/EtOAc (100:0-90:10) to afford 3-(2,6-bis(benzyloxy)pyridin-3-yl)-6-bromo-4-methylbenzo[d]oxazol-2(3H)-one (3.00 g, 6.60%) as yellow solid.

### Step 4: 3-(2,6-bis(benzyloxy)pyridin-3-yl)-4-methyl-6-(1,4-dioxa-8-azaspiro[4.5]decan-8-yl)benzo[d]oxazol-2(3H)-one

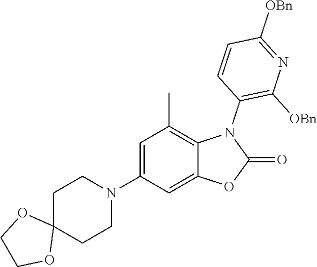 [SMILES: Cc1cc(N2CCC3(CC2)OCCO3)cc2oc(=O)n(-c3ccc(OCc4ccccc4)nc3OCc3ccccc3)c12]

Into a 250-mL round-bottom flask purged and maintained with N₂, were added 3-(2,6-bis(benzyloxy)pyridin-3-yl)-6-bromo-4-methylbenzo[d]oxazol-2(3H)-one (1.60 g, 3.10 mmol), 1,4-dioxa-8-azaspiro[4.5]decane (0.67 g, 4.65 mmol), Cs₂CO₃ (3.02 g, 9.30 mmol), Ruphos (289.5 mg, 0.62 mmol), Pd₂(dba)₃ (283.7 mg, 0.31 mmol) and Dioxane (50 mL) at room temperature. The reaction was stirred for 2 h at 100° C. The resulting mixture was concentrated under vacuum. The residue was purified by silica gel column chromatography, eluted with PE/EtOAc (100:0-70:30) to afford 3-(2,6-bis(benzyloxy)pyridin-3-yl)-4-methyl-6-(1,4-dioxa-8-azaspiro[4.5]decan-8-yl)benzo[d]oxazol-2(3H)-one (710.0 mg, 39.55%) as yellow solid.

### Step 5: 3-(4-methyl-2-oxo-6-(1,4-dioxa-8-azaspiro[4.5]decan-8-yl)benzo[d]oxazol-3(2H)-yl)piperidine-2,6-dione

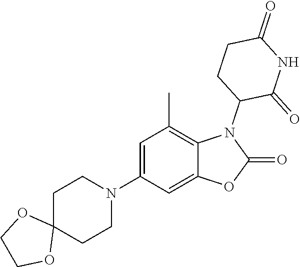 [SMILES: Cc1cc(N2CCC3(CC2)OCCO3)cc2oc(=O)n(C3CCC(=O)NC3=O)c12]

Into a 250-mL round-bottom flask purged and maintained with H₂, were added 3-(2,6-bis(benzyloxy)pyridin-3-yl)-4-methyl-6-(1,4-dioxa-8-azaspiro[4.5]decan-8-yl)benzo[d]oxazol-2(3H)-one (710.0 mg, 1.24 mmol), Pd/C (710.0 mg) and THF (30 mL) at room temperature. The reaction was stirred for 15 h at 45° C. The precipitated solids were collected by filtration and washed with THF (300 mL). The residue was purified by silica gel column chromatography, eluted with PE/EtOAc (100:0-50:50) to afford 3-(4-methyl-2-oxo-6-(1,4-dioxa-8-azaspiro[4.5]decan-8-yl)benzo[d]oxazol-3(2H)-yl)piperidine-2,6-dione (320.0 mg, 65.08%) as yellow solid.

### Step 6: 3-(4-methyl-2-oxo-6-(4-oxopiperidin-1-yl)benzo[d]oxazol-3(2H)-yl)piperidine-2,6-dione

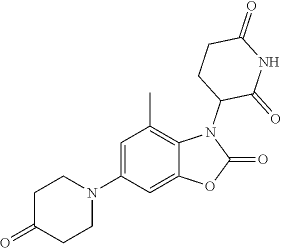 [SMILES: Cc1cc(N2CCC(=O)CC2)cc2oc(=O)n(C3CCC(=O)NC3=O)c12]

Into a 100-mL round-bottom flask were added 3-(4-methyl-2-oxo-6-(1,4-dioxa-8-azaspiro[4.5]decan-8-yl)benzo[d]oxazol-3(2H)-yl)piperidine-2,6-dione (280.0 mg, 0.70 mmol) and TFA (10 mL) at room temperature. The resulting mixture was stirred for 15 h at 60° C. The reaction was quenched by the addition of ice water (50 mL). The resulting mixture was extracted with EtOAc (2×100 mL), dried over anhydrous Na₂SO₄. The resulting mixture was concentrated under reduced pressure to afford 3-(4-methyl-2-oxo-6-(4-oxopiperidin-1-yl)benzo[d]oxazol-3(2H)-yl)piperidine-2,6-dione 2,2,2-trifluoroacetate (120.9 mg, 36.49%) as pink solid.

### Step 7: (1S,3R)-3-(3-((2-(1′-(3-((R)-2,6-dioxopiperidin-3-yl)-4-methyl-2-oxo-2,3-dihydrobenzo[d]oxazol-6-yl)-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

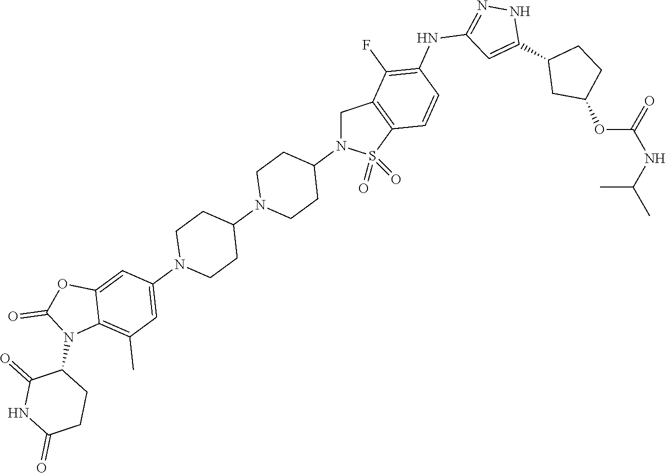 [SMILES: Cc1cc(N2CCC(N3CCC(N4Cc5c(ccc(Nc6cc([C@@H]7CC[C@H](OC(=O)NC(C)C)C7)[nH]n6)c5F)S4(=O)=O)CC3)CC2)cc2oc(=O)n([C@@H]3CCC(=O)NC3=O)c12]

The racemic compound was synthesized in the procedures similar to Example 63, which was further separated by Chiral Prep-HPLC to afford the desired product:

Example 276 (98.1% ee); Retention time: 9.38 min. ¹H NMR (500 MHz, DMSO-d₆) δ 11.98 (s, 1H), 11.13 (s, 1H), 8.76 (s, 1H), 8.30 (t, J=5 Hz, 1H), 7.50 (t, J=10 Hz, 1H), 6.91-6.85 (m, 1H), 6.79-6.72 (m, 1H), 6.63-6.59 (m, 1H), 5.83 (s, 1H), 5.31-5.23 (m, 1H), 5.01-4.94 (m, 1H), 4.51 (s, 2H), 3.72-3.62 (m, 2H), 3.47-3.37 (m, 1H), 3.11-3.01 (m, 1H), 2.99-2.93 (m, 2H), 2.91-2.82 (m, 1H), 2.68-2.58 (m, 4H), 2.45 (s, 3H), 2.43-2.42 (m, 1H), 2.32-2.23 (m, 3H), 2.05-1.98 (m, 3H), 1.96-1.54 (m, 10H), 1.36-1.11 (m, 2H), 1.09-0.95 (m, 6H). LC-MS (ESI): m/z [M+H]⁺=862.

Chiral analytical method: i-Cellulose-5, 4.6 mm×250 mm, 5 um; Mobile phase: A for MtBE (0.1% 2M NH3 MeOH) and B for DCM:MeOH (50:50); Gradient: Mobile Phase A: Mobile Phase B=30:70 (v/v); HPLC Equipment: HPLC-Agilent, Back pressure: 100 bar; Column temperature: 35° C.

Chiral Prep-HPLC Condition: Column: i-Cellulose-5, 250 mm×20 mm, 5 um; Mobile phase: A for MtBE (0.1% 2M NH3 MeOH) and B for DCM:MeOH (50:50); Gradient: Mobile Phase A: Mobile Phase B=30:70 (v/v); Flow Rate: 20 mL/min, Wave Length: UV 200 nm and 270 nm, Prep-HPLC Equipment: Prep-HPLC-Gilson, Back pressure: 100 bar; Column temperature: 25° C.

### Example 277 and 278: (1S,3R)-3-(3-((2-(1-((1-(3-((S)-2,6-dioxopiperidin-3-yl)-4-fluoro-2-oxo-2,3-dihydrobenzo[d]oxazol-6-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-(1-((1-(3-((R)-2,6-dioxopiperidin-3-yl)-4-fluoro-2-oxo-2,3-dihydrobenzo[d]oxazol-6-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

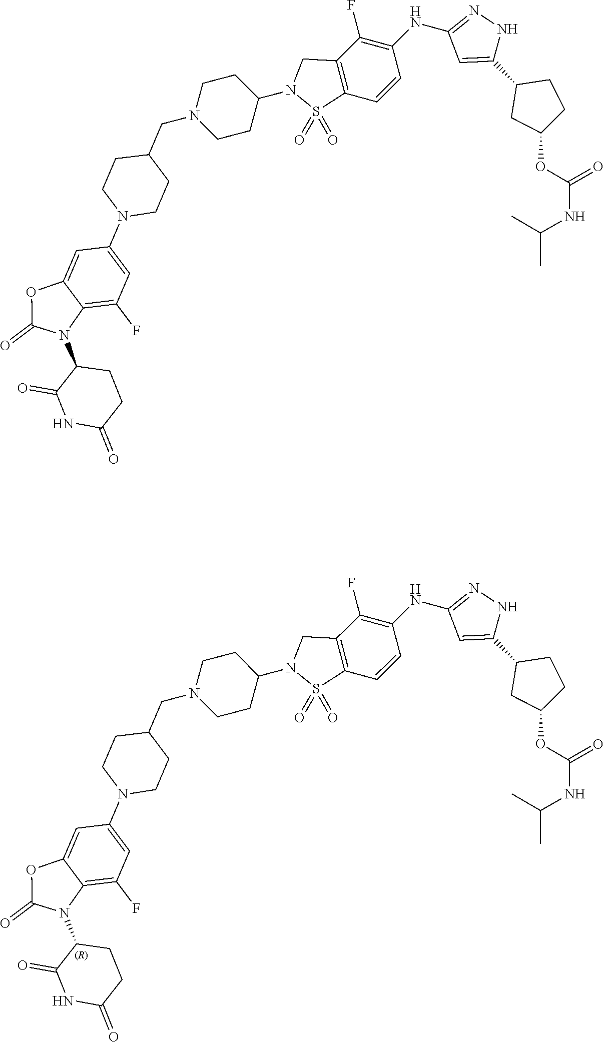

### Step 1: 3-(2,6-bis(benzyloxy)pyridin-3-yl)-6-(4-(dimethoxymethyl)piperidin-1-yl)-4-fluorobenzo[d]oxazol-2(3H)-one

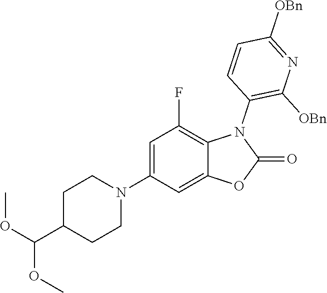 [SMILES: COC(OC)C1CCN(c2cc(F)c3c(c2)oc(=O)n3-c2ccc(OCc3ccccc3)nc2OCc2ccccc2)CC1]

To a solution of 3-(2,6-bis(benzyloxy)pyridin-3-yl)-6-bromo-4-fluorobenzo[d]oxazol-2(3H)-one (1.7 g, 3.26 mmol) in dioxane (80 mL) were added 4-(dimethoxymethyl)piperidine (778 mg, 4.89 mmol), Pd-PEPPSI-IPentCl 2-methylpyridine (137 mg, 0.16 mmol) and Cs₂CO₃ (2 g, 6.52 mmol). The resulting solution was stirred for 3 h at 100° C. under nitrogen atmosphere. The resulting mixture was concentrated under vacuum. The residue was purified by combi-flash (EtOAc/PE=80%) to give the product (320 mg, 16.3%) as red solid. LCMS (ESI) m/e [M+1]⁺=600.

### Step 2: 3-(6-(4-(dimethoxymethyl)piperidin-1-yl)-4-fluoro-2-oxobenzo[d]oxazol-3(2H)-yl)piperidine-2,6-dione

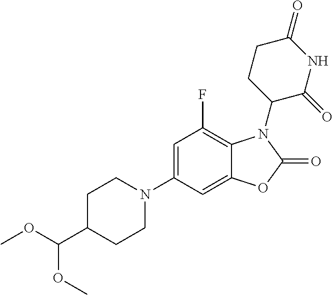 [SMILES: COC(OC)C1CCN(c2cc(F)c3c(c2)oc(=O)n3C2CCC(=O)NC2=O)CC1]

To a solution of 3-(2,6-bis(benzyloxy)pyridin-3-yl)-6-(4-(dimethoxymethyl)piperidin-1-yl)-4-fluorobenzo[d]oxazol-2(3H)-one (300 mg, 0.5 mmol) in THF (10 mL) was added Pd/C (300 mg, 10% wt), After stirring of the reaction mixture for 16 h at 50° C. under H₂ atmosphere. After filtration, the filtrate was concentrated under vacuum. The residue was the product (158.9 mg, 75.4%) as grey solid. LCMS (ESI) m/e [M+1]⁺=422.

### Step 3: 1-(3-(2,6-dioxopiperidin-3-yl)-4-fluoro-2-oxo-2,3-dihydrobenzo[d]oxazol-6-yl)piperidine-4-carbaldehyde

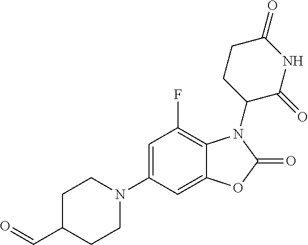 [SMILES: O=CC1CCN(c2cc(F)c3c(c2)oc(=O)n3C2CCC(=O)NC2=O)CC1]

To a flask was added 3-(6-(4-(dimethoxymethyl)piperidin-1-yl)-4-fluoro-2-oxobenzo[d]oxazol-3(2H)-yl)piperidine-2,6-dione (100 mg, 0.24 mmol) and formic acid (5 mL). The mixture was stirred at room temperature for 2 h. The solution was concentrated under reduced pressure to give the crude product. LC-MS (ESI): m/z [M+H]⁺=376.4.

### Step 4: (1S,3R)-3-(3-((2-(1-((1-(3-((S)-2,6-dioxopiperidin-3-yl)-4-fluoro-2-oxo-2,3-dihydrobenzo[d]oxazol-6-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-(1-((1-(3-((R)-2,6-dioxopiperidin-3-yl)-4-fluoro-2-oxo-2,3-dihydrobenzo[d]oxazol-6-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

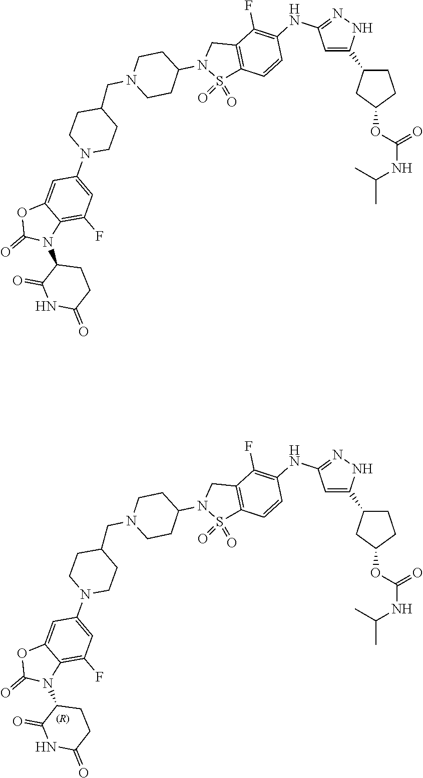

The racemic compound was synthesized in the procedures similar to Example 62, which was further separated by Chiral Prep-HPLC to afford the desired product:

Enantiomer 1 (Example 277, 99.1% ee); Retention time: 6.92 min. ¹H NMR (500 MHz, DMSO-d₆) δ 12.00 (s, 1H), 11.20 (s, 1H), 8.77 (s, 1H), 8.30 (t, J=10 Hz, 1H), 7.52 (t, J=10 Hz, 1H), 7.15-6.97 (m, 2H), 6.87-6.81 (m, 1H), 5.83 (s, 1H), 5.40-5.29 (m, 1H), 5.04-4.96 (m, 1H), 4.52 (s, 2H), 3.78-3.53 (m, 4H), 3.46-3.40 (m, 1H), 3.14-3.02 (m, 2H), 3.00-2.84 (m, 3H), 2.76-2.60 (m, 4H), 2.46-2.40 (m, 1H), 2.28-2.21 (m, 2H), 2.08-1.97 (m, 3H), 1.96-1.54 (m, 10H), 1.36-1.11 (m, 2H), 1.09-0.95 (m, 6H). LC-MS (ESI): m/z [M+H]⁺=880.

Enantiomer 2 (Example 278, 97.3% ee); Retention time: 9.13 min. ¹H NMR (500 MHz, DMSO-d₆) δ 12.00 (s, 1H), 11.20 (s, 1H), 8.77 (s, 1H), 8.30 (t, J=10 Hz, 1H), 7.52 (t, J=10 Hz, 1H), 7.15-6.97 (m, 2H), 6.87-6.81 (m, 1H), 5.83 (s, 1H), 5.40-5.29 (m, 1H), 5.04-4.96 (m, 1H), 4.52 (s, 2H), 3.78-3.53 (m, 4H), 3.46-3.40 (m, 1H), 3.14-3.02 (m, 2H), 3.00-2.84 (m, 3H), 2.76-2.60 (m, 4H), 2.46-2.40 (m, 1H), 2.28-2.21 (m, 2H), 2.08-1.97 (m, 3H), 1.96-1.54 (m, 10H), 1.36-1.11 (m, 2H), 1.09-0.95 (m, 6H). LC-MS (ESI): m/z [M+H]⁺=880.

Chiral analytical method: i-Cellulose-5, 4.6 mm×250 mm, 5 um; Mobile phase: A for MtBE (0.1% 2M NH3 MeOH) and B for DCM:MeOH (50:50); Gradient: Mobile Phase A: Mobile Phase B=30:70 (v/v); HPLC Equipment: HPLC-Agilent, Back pressure: 100 bar; Column temperature: 35° C.

Chiral Prep-HPLC Condition: Column: i-Cellulose-5, 250 mm×20 mm, 5 um; Mobile phase: A for MtBE (0.1% 2M NH3 MeOH) and B for DCM:MeOH (50:50); Gradient: Mobile Phase A: Mobile Phase B=30:70 (v/v); Flow Rate: 20 mL/min, Wave Length: UV 200 nm and 270 nm, Prep-HPLC Equipment: Prep-HPLC-Gilson, Back pressure: 100 bar; Column temperature: 25° C.

### Example 279: (1S,3R)-3-(3-((2-(1′-(3-(2,6-dioxopiperidin-3-yl)-4-fluoro-2-oxo-2,3-dihydrobenzo[d]oxazol-6-yl)-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

### Step 1: 6-bromo-4-fluorobenzo[d]oxazol-2(3H)-one

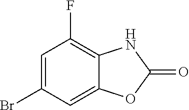 [SMILES: O=c1[nH]c2c(F)cc(Br)cc2o1]

To a solution of 2-amino-5-bromo-3-fluorophenol (10 g, 48.5 mmol) in THF (300 mL) was added CDI (12 g, 72.8 mmol), The reaction solution was stirred for 2 h at 70° C. The resulting mixture was concentrated under vacuum. The residue was purified by combi-flash (MeOH/DCM=25%) to give the product (9 g, 79.9%) as red solid. LCMS (ESI) m/e [M+1]⁺=233

### Step 2: 3-(2,6-bis(benzyloxy)pyridin-3-yl)-6-bromo-4-fluorobenzo[d]oxazol-2(3H)-one

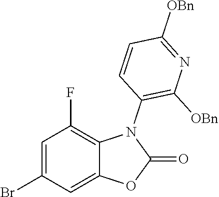 [SMILES: O=c1oc2cc(Br)cc(F)c2n1-c1ccc(OCc2ccccc2)nc1OCc1ccccc1]

To a solution of 6-bromo-1-methyl-1,3-dihydro-2H-benzo[d]imidazol-2-one (9 g, 38.7 mmol) in dioxane (300 mL) were added 2,6-bis(benzyloxy)-3-(4,4,5,5-tetramethyl-1,3,2-dioxaborolan-2-yl)pyridine (24.2 g, 58.1 mmol), Cu(OAc)₂ (7 g, 38.7 mmol), Pyridine (9 g, 116.3 mmol) and 4A MS (9 g). The resulting solution was stirred for 20 h at 80° C. under oxygen atmosphere. After filtration, the filtrate was concentrated under vacuum. The residue was purified by combi-flash (EtOAc/PE=30%) to give the product (2.2 g, 24.4%) as white solid. LCMS (ESI) m/e [M+1]⁺=521

### Step 3: 3-(2,6-bis(benzyloxy)pyridin-3-yl)-4-fluoro-6-(1,4-dioxa-8-azaspiro[4.5]decan-8-yl)benzo[d]oxazol-2(3H)-one

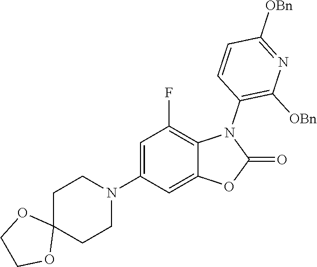 [SMILES: O=c1oc2cc(N3CCC4(CC3)OCCO4)cc(F)c2n1-c1ccc(OCc2ccccc2)nc1OCc1ccccc1]

To a solution of 3-(2,6-bis(benzyloxy)pyridin-3-yl)-6-bromo-4-fluorobenzo[d]oxazol-2(3H)-one (2.1 g, 4.0 mmol) in dioxane (80 mL) were added 4-(dimethoxymethyl)piperidine (858 mg, 6.0 mmol), Pd-PEPPSI-IPentCl 2-methylpyridine (168 mg, 0.2 mmol) and Cs₂CO₃ (2.6 g, 8.0 mmol). The resulting solution was stirred for 3 h at 100° C. under nitrogen atmosphere. The resulting mixture was concentrated under vacuum. The residue was purified by combi-flash (EtOAc/PE=56%) to give the product (400 mg, 17.0%) as yellow solid. LCMS (ESI) m/e [M+1]⁺=584

### Step 4: 3-(4-fluoro-2-oxo-6-(1,4-dioxa-8-azaspiro[4.5]decan-8-yl)benzo[d]oxazol-3(2H)-yl)piperidine-2,6-dione

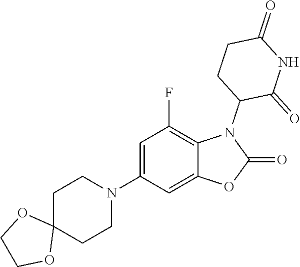 [SMILES: O=C1CCC(n2c(=O)oc3cc(N4CCC5(CC4)OCCO5)cc(F)c32)C(=O)N1]

To a solution of 3-(2,6-bis(benzyloxy)pyridin-3-yl)-4-fluoro-6-(1,4-dioxa-8-azaspiro[4.5]decan-8-yl)benzo[d]oxazol-2(3H)-one (400 mg, 0.9 mmol) in THF (30 mL) was added Pd/C (400 mg, 10% wt). The resulting solution was stirred for 12 h at 50° C. under H₂ atmosphere. After filtration, the filtrate was concentrated under vacuum. The residue was the product (250 mg, 89.9%) as white solid. LCMS (ESI) m/e [M+1]⁺=406

### Step 5: 3-(5-(4-(dimethoxymethyl)piperidin-1-yl)-3-methyl-2-oxo-2,3-dihydro-1H-benzo[d]imidazol-1-yl)piperidine-2,6-dione

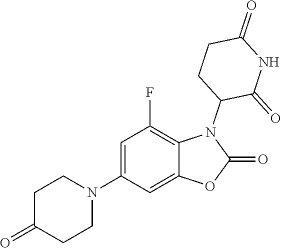 [SMILES: O=C1CCN(c2cc(F)c3c(c2)oc(=O)n3C2CCC(=O)NC2=O)CC1]

The solution of 3-(4-fluoro-2-oxo-6-(1,4-dioxa-8-azaspiro[4.5]decan-8-yl)benzo[d]oxazol-3(2H)-yl)piperidine-2,6-dione (250 mg, 4.8 mmol) in H₂SO₄ (20 mL) at 0° C. The resulting solution was stirred for 3 h at rt. The residue was basified to pH 5 with Na₂CO₃. The resulting mixture was extracted with EtOAc (3×20 mL). The combined organic layers were washed with brine (3×20 mL), dried over anhydrous Na₂SO₄. After filtration, the filtrate was concentrated under reduced pressure. The residue was the product (113.4 mg, 65.4%) as dark green solid. LCMS (ESI) m/e [M+1]⁺=362

### Step 6: (1S,3R)-3-(3-((2-(1′-(3-(2,6-dioxopiperidin-3-yl)-2-oxo-2,3-dihydrobenzo[d]oxazol-6-yl)-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

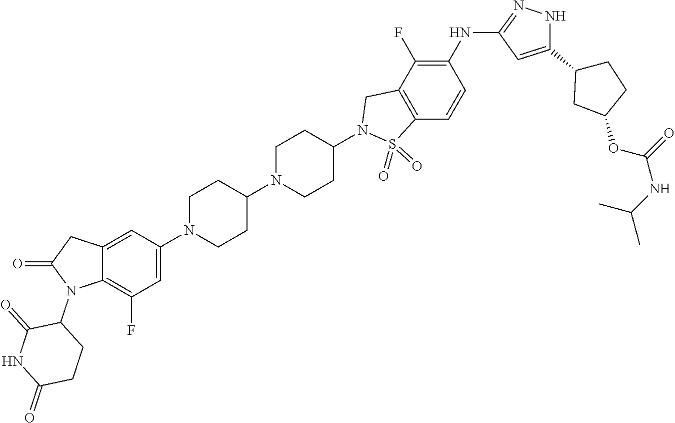 [SMILES: CC(C)NC(=O)O[C@H]1CC[C@@H](c2cc(Nc3ccc4c(c3F)CN(C3CCN(C5CCN(c6cc(F)c7c(c6)CC(=O)N7C6CCC(=O)NC6=O)CC5)CC3)S4(=O)=O)n[nH]2)C1]

The titled compound was synthesized in the procedures similar to Example 62. ¹H NMR (500 MHz, DMSO-d₆) δ 11.99 (s, 1H), 11.20 (s, 1H), 8.76 (s, 1H), 8.29 (s, 1H), 7.50 (d, J=10 Hz, 1H), 7.00-6.90 (m, 2H), 6.76-6.69 (m, 1H), 5.83 (s, 1H), 5.42-5.29 (m, 1H), 5.04-4.96 (m, 1H), 4.50 (s, 2H), 3.79-2.68 (m, 2H), 3.63-3.52 (m, 1H), 3.44-3.38 (m, 1H), 3.11-3.01 (m, 1H), 3.00-2.89 (m, 2H), 2.73-2.60 (m, 4H), 2.45-2.40 (m, 1H), 2.31-2.20 (m, 3H), 2.08-1.97 (m, 1H), 1.97-1.64 (m, 9H), 1.64-1.45 (m, 3H), 1.11-0.95 (m, 6H). LC-MS (ESI): m/z [M+H]⁺=866.

### Example 279a and 279b: (1S,3R)-3-(3-((2-(1′-(3-((S)-2,6-dioxopiperidin-3-yl)-4-fluoro-2-oxo-2,3-dihydrobenzo[d]oxazol-6-yl)-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-(1′-(3-((R)-2,6-dioxopiperidin-3-yl)-4-fluoro-2-oxo-2,3-dihydrobenzo[d]oxazol-6-yl)-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

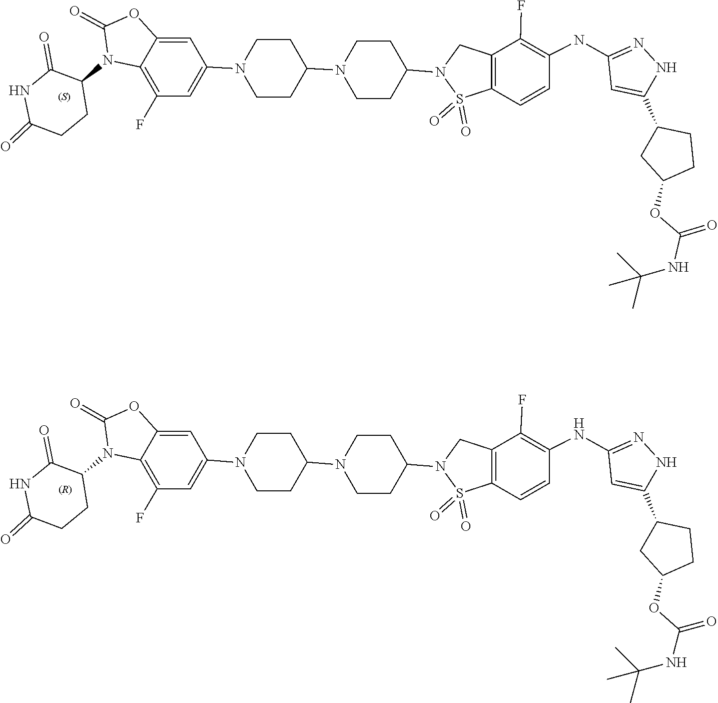

The racemic product was synthesized in the procedures similar to Example 63, which was further separated by Chiral-Prep-HPLC to afford the desired product:

Enantiomer 1 (Example 279a, 99% ee); Retention time: 7.74 min ¹H NMR (500 MHz, DMSO-d₆) δ 11.99 (s, 1H), 11.20 (s, 1H), 8.76 (s, 1H), 8.29 (s, 1H), 7.50 (d, J=10 Hz, 1H), 7.00-6.90 (m, 2H), 6.76-6.69 (m, 1H), 5.83 (s, 1H), 5.42-5.29 (m, 1H), 5.04-4.96 (m, 1H), 4.50 (s, 2H), 3.79-2.68 (m, 2H), 3.63-3.52 (m, 1H), 3.44-3.38 (m, 1H), 3.11-3.01 (m, 1H), 3.00-2.89 (m, 2H), 2.73-2.60 (m, 4H), 2.45-2.40 (m, 1H), 2.31-2.20 (m, 3H), 2.08-1.97 (m, 1H), 1.97-1.64 (m, 9H), 1.64-1.45 (m, 3H), 1.11-0.95 (in, 6H). LC-MS (ESI): m/z [M+H]⁺ 866.

Enantiomer 2 (Example 279b, 97.3% ee); Retention time: 9.81 min ¹H NMR (500 MHz, DMSO-d₆) δ 11.99 (s, 1H), 11.20 (s, 1H), 8.76 (s, 1H), 8.29 (s, 1H), 7.50 (d, J=10 Hz, 1H), 7.00-6.90 (m, 2H), 6.76-6.69 (m, 1H), 5.83 (s, 1H), 5.42-5.29 (m, 1H), 5.04-4.96 (m, 1H), 4.50 (s, 2H), 3.79-2.68 (m, 2H), 3.63-3.52 (m, 1H), 3.44-3.38 (m, 1H), 3.11-3.01 (m, 1H), 3.00-2.89 (m, 2H), 2.73-2.60 (m, 4H), 2.45-2.40 (m, 1H), 2.31-2.20 (m, 3H), 2.08-1.97 (m, 1H), 1.97-1.64 (m, 9H), 1.64-1.45 (m, 3H), 1.11-0.95 (m, 6H). LC-MS (ESI): m/z [M+H]⁺=866.

Chiral analytical method: Column: I-Cellulose-5, 4.6 mm×250 mm, 5 um; Mobile phase: A for MeBE (0.1% 2M NH3 MeOH) and B for DCM/MeOH (50:50); Gradient: Mobile Phase A: Mobile Phase B=30:70 (v/v); HPLC Equipment: HPLC-Agilent, Back pressure: 100 bar; Column temperature: 35° C.

Chiral Prep-HPLC Condition: Column: I-Cellulose-5, 20 mm×250 mm, 5 um; Mobile phase: A for MeBE (0.1% 2M NH3 MeOH) and B for DCM/MeOH (50:50); Gradient: Mobile Phase A: Mobile Phase B=30:70 (v/v); Flow Rate: 20 mL/min, Wave Length: UV 200 nm and 270 nm, Prep-HPLC Equipment: Prep-HPLC-Gilson, Back pressure: 100 bar; Column temperature: 25° C.

### Example 280 and 281: (1S,3R)-3-(3-((2-(1-((1-(3-((R)-2,6-dioxopiperidin-3-yl)-4-fluoro-7-methyl-2-oxo-2,3-dihydrobenzo[d]oxazol-6-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-(1-((1-(3-((S)-2,6-dioxopiperidin-3-yl)-4-fluoro-7-methyl-2-oxo-2,3-dihydrobenzo[d]oxazol-6-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

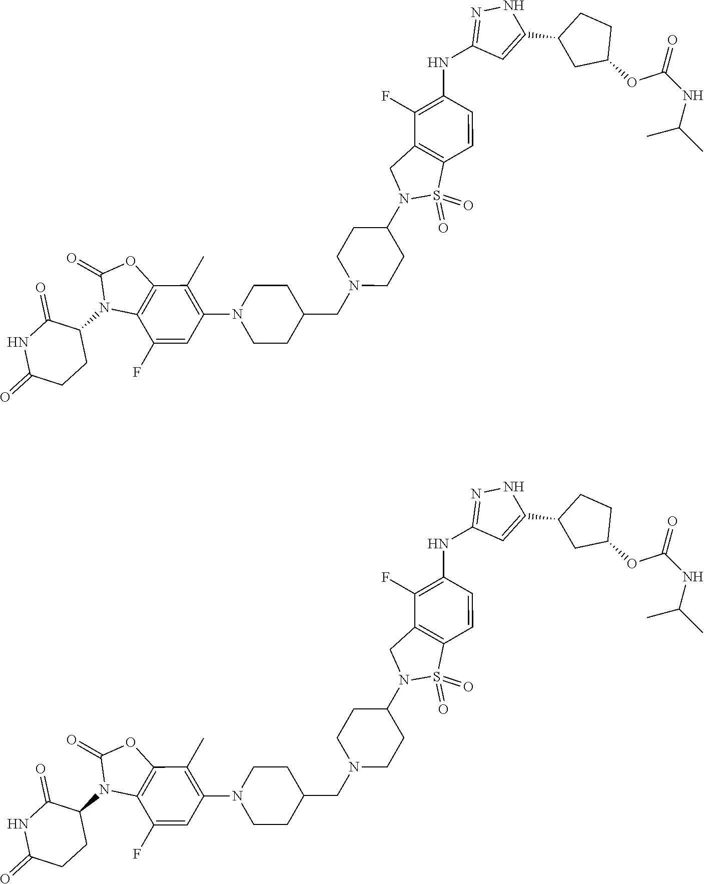 [SMILES: Cc1c(N2CCC(CN3CCC(N4Cc5c(ccc(Nc6cc([C@@H]7CC[C@H](OC(=O)NC(C)C)C7)[nH]n6)c5F)S4(=O)=O)CC3)CC2)cc(F)c2c1oc(=O)n2[C@@H]1CCC(=O)NC1=O ; Cc1c(N2CCC(CN3CCC(N4Cc5c(ccc(Nc6cc([C@@H]7CC[C@H](OC(=O)NC(C)C)C7)[nH]n6)c5F)S4(=O)=O)CC3)CC2)cc(F)c2c1oc(=O)n2[C@H]1CCC(=O)NC1=O]

### Step 1: 2-amino-3-fluoro-6-methylphenol

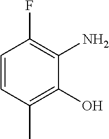

To a solution of 3-fluoro-6-methyl-2-nitrophenol (5 g, 29.2 mmol) in THF (100 mL) was added Pd/C (5 g, 10% wt). The resulting solution was stirred for 12 h at rt under H₂ atmosphere. After filtration, the filtrate was concentrated under vacuum. The residue was the product (4.8 g, 93.5%) as yellow solid. LCMS (ESI) m/e [M+1]⁺=142

### Step 2: 4-fluoro-7-methylbenzo[d]oxazol-2(3H)-one

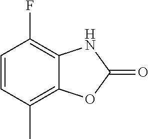

To a solution of 2-amino-3-fluoro-6-methylphenol (4.8 g, 3.38 mmol) in ACN (100 mL) was added CDI (8.2 g, 5.1 mmol), The reaction solution was stirred for 16 h at 70° C. The resulting mixture was concentrated under vacuum. The residue was purified by combi-flash (EA/PE=80%) to give the product (4 g, 70.8%) as brown solid. LCMS (ESI) m/e [M+1]⁺=168

### Step 3: 6-bromo-4-fluoro-7-methylbenzo[d]oxazol-2(3H)-one

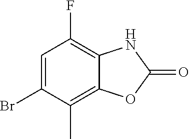

To a solution of 4-fluoro-7-methylbenzo[d]oxazol-2(3H)-one (4 g, 2.38 mmol) in DMF (50 mL) at 0° C. was added NBS (4.2 g, 2.38 mmol). After stirring of the reaction mixture for 2 h at 0° C. The resulting mixture quenched with water. The resulting mixture was extracted with EA (3×20 mL). The organic layers were washed with brine and concentrated under reduced pressure. The reaction was concentrated under vacuum. The crude was purified by combi-flash (EA/PE=50%). The product (4.1 g, 70%) as yellow solid. LCMS (ESI) m/e [M+1]⁺=246

### Step 4: 3-(6-bromo-4-fluoro-7-methyl-2-oxobenzo[d]oxazol-3(2H)-yl)-1-(4-methoxybenzyl)piperidine-2,6-dione

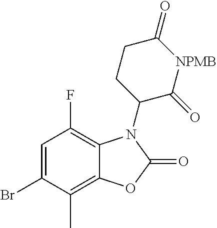

To a solution of 6-bromo-4-fluoro-7-methylbenzo[d]oxazol-2(3H)-one (4 g, 1.62 mmol) in THF (300 mL) were added t-BuOK (2.2 g, 1.94 mmol) at 0° C. The resulting solution was stirred for 0.5 h at 0° C. To the above mixture was added 1-(4-methoxybenzyl)-2,6-dioxopiperidin-3-yltrifluoromethanesulfonate (3 g, 8.1 mmol) in portions at 0° C. The resulting mixture was stirred for additional 16 h at rt. The reaction was concentrated under vacuum. The crude was purified by combine-flash (EA/PE=70%). The product (6 g, 77.3%) as red solid. LCMS (ESI) m/e [M+1]⁺=477

### Step 5: 3-(6-bromo-4-fluoro-7-methyl-2-oxobenzo[d]oxazol-3(2H)-yl)piperidine-2,6-dione

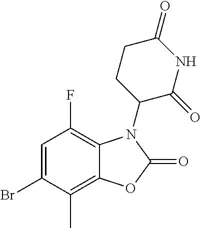 [SMILES: Cc1c(Br)cc(F)c2c1oc(=O)n2C1CCC(=O)NC1=O]

To a solution of 3-(6-bromo-4-fluoro-7-methyl-2-oxobenzo[d]oxazol-3(2H)-yl)-1-(4-methoxybenzyl)piperidine-2,6-dione (6 g, 12.5 mmol) in TFA (30 mL) were added TfOH (18.7 g, 125 mmol). The resulting solution was stirred for 16 h at 65° C. The resulting mixture was concentrated under vacuum. The residue was purified by combi-flash (EtOAc/PE=80%) to give the product (4.6 g, 98%) as green solid. LCMS (ESI) m/e [M+1]⁺=357.

### Step 6: 3-(6-(4-(dimethoxymethyl)piperidin-1-yl)-4-fluoro-7-methyl-2-oxobenzo[d]oxazol-3(2H)-yl)piperidine-2,6-dione

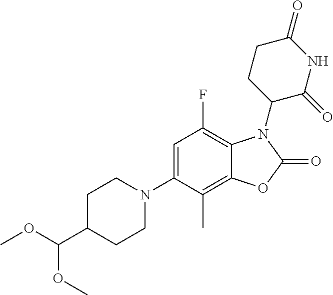 [SMILES: COC(OC)C1CCN(c2cc(F)c3c(oc(=O)n3C3CCC(=O)NC3=O)c2C)CC1]

To a solution of 3-(6-bromo-4-fluoro-7-methyl-2-oxobenzo[d]oxazol-3(2H)-yl)piperidine-2,6-dione (1.5 g, 4.2 mmol) in dioxane (20 mL) were added 4-(dimethoxymethyl)piperidine (1 g, 11.6 mmol), Pd-PEPPSI-IPentCl 2-methylpyridine (176.4 mg, 0.2 mmol) and Cs₂CO₃ (3.4 g, 10.5 mmol). The resulting solution was stirred for 16 h at 100° C. under nitrogen atmosphere. The resulting mixture was concentrated under vacuum. The residue was purified by combi-flash (EtOAc/PE=80%) to give the product (177.1 mg, 9.6%) as brown solid. LCMS (ESI) m/e [M+1]⁺=436.

### Step 7: 1-(3-(2,6-dioxopiperidin-3-yl)-4-fluoro-7-methyl-2-oxo-2,3-dihydrobenzo[d]oxazol-6-yl)piperidine-4-carbaldehyde

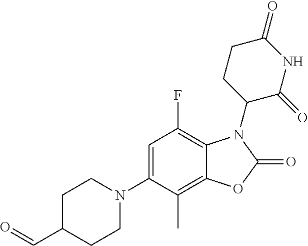 [SMILES: Cc1c(N2CCC(C=O)CC2)cc(F)c2c1oc(=O)n2C1CCC(=O)NC1=O]

To a flask was added 3-(6-(4-(dimethoxymethyl)piperidin-1-yl)-4-fluoro-2-oxobenzo[d]oxazol-3(2H)-yl)piperidine-2,6-dione (100 mg, 0.23 mmol) and formic acid (5 mL). The mixture was stirred at room temperature for 2 h. The solution was concentrated under reduced pressure to give the crude product. LC-MS (ESI): m/z [M+H]⁺=390.4.

### Step 8: (1S,3R)-3-(3-((2-(1-((1-(3-((R)-2,6-dioxopiperidin-3-yl)-4-fluoro-7-methyl-2-oxo-2,3-dihydrobenzo[d]oxazol-6-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-(1-((1-(3-((S)-2,6-dioxopiperidin-3-yl)-4-fluoro-7-methyl-2-oxo-2,3-dihydrobenzo[d]oxazol-6-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

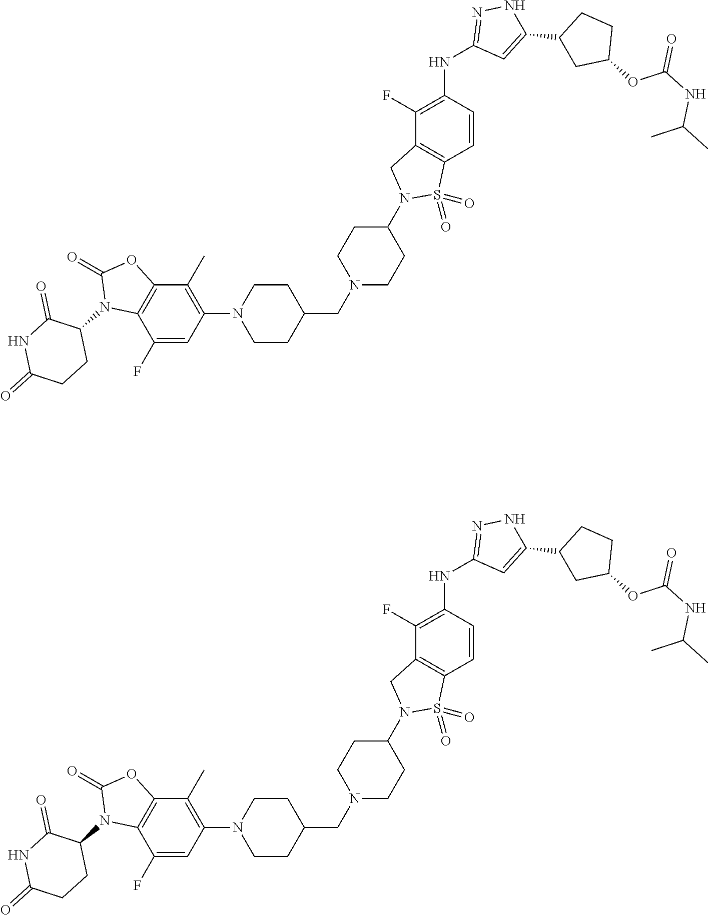

The racemic product was synthesized in the procedures similar to Example 62, which was further separated by Chiral Prep-HPLC to afford the desired product:

Enantiomer 1 (Example 281, 98.6% ee); Retention time: 3.26 min. 1H NMR (500 MHz, DMSO-d6) δ 11.99 (s, 1H), 11.21 (s, 1H), 8.76 (s, 1H), 8.30 (t, J=5 Hz, 1H), 7.51 (d, J=5 Hz, 1H), 6.97-6.91 (m, 1H), 6.88-6.76 (m, 1H), 5.83 (s, 1H), 5.45-5.30 (m, 1H), 5.05-4.95 (m, 1H), 4.51 (s, 2H), 3.63-3.51 (m, 1H), 3.47-3.39 (m, 1H), 3.11-2.86 (m, 6H), 2.65-2.57 (m, 3H), 2.47-2.43 (m, 1H), 2.28-2.15 (m, 1H), 2.11-1.97 (m, 3H), 1.96-1.78 (m, 7H), 1.77-1.65 (m, 2H), 1.66-1.55 (m, 1H), 1.36-1.21 (m, 3H), 1.09-0.97 (m, 6H). LC-MS (ESI): m/z [M+H]⁺=894.

Enantiomer 2 (Example 280, 90% ee); Retention time: 3.97 min. 1H NMR (500 MHz, DMSO-d6) δ 11.99 (s, 1H), 11.21 (s, 1H), 8.76 (s, 1H), 8.30 (t, J=5 Hz, 1H), 7.51 (d, J=5 Hz, 1H), 6.97-6.91 (m, 1H), 6.88-6.76 (m, 1H), 5.83 (s, 1H), 5.45-5.30 (m, 1H), 5.05-4.95 (m, 1H), 4.51 (s, 2H), 3.63-3.51 (m, 1H), 3.47-3.39 (m, 1H), 3.11-2.86 (m, 6H), 2.65-2.57 (m, 3H), 2.47-2.43 (m, 1H), 2.28-2.15 (m, 1H), 2.11-1.97 (m, 3H), 1.96-1.78 (m, 7H), 1.77-1.65 (m, 2H), 1.66-1.55 (m, 1H), 1.36-1.21 (m, 3H), 1.09-0.97 (m, 6H). LC-MS (ESI): m/z [M+H]⁺=894.

Chiral analytical method: Column: IH (4.6 mm×150 mm, 5 um); Mobile phase: A for MtBE (0.2% NH3·MeOH) and B for DCM:MeOH=50:50; Gradient: Mobile Phase A: Mobile Phase B=70:30 (v/v); HPLC Equipment: HPLC-Agilent, Back pressure: 100 bar; Column temperature: 35° C.

Chiral Prep-HPLC Condition: Column: IH, 250*20.0 mm, 5 um; Mobile phase: A for MtBE (0.2% NH3·MeOH) and B for DCM:MeOH=50:50; Gradient: Mobile Phase A: Mobile Phase B=80:20 (v/v); Flow Rate: 20 mL/min, Wave Length: UV 200 nm and 270 nm, Prep-HPLC Equipment: Prep-HPLC-Gilson, Back pressure: 100 bar; Column temperature: 25° C.

### Example 282 and 283: (1S,3R)-3-(3-((2-(1-((1-(3-((S)-2,6-dioxopiperidin-3-yl)-4,7-difluoro-2-oxo-2,3-dihydrobenzo[d]oxazol-6-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-(1-((1-(3-((R)-2,6-dioxopiperidin-3-yl)-4,7-difluoro-2-oxo-2,3-dihydrobenzo[d]oxazol-6-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

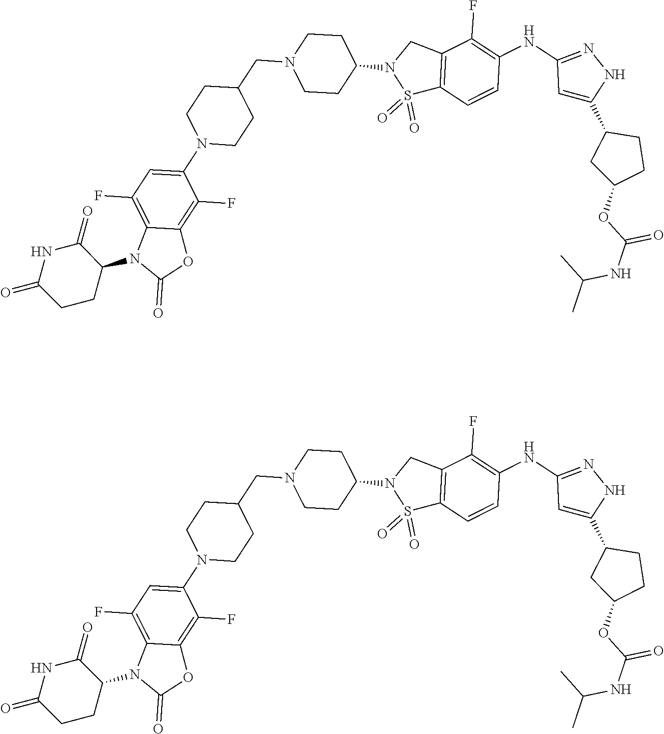

### Step 1: 2-amino-3,6-difluoropheno

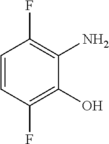

Into a 500-mL round-bottom flask purged and maintained with N₂, were added 3,6-difluoro-2-nitrophenol (10.00 g, 57.14 mmol), Fe (9.60 g, 171.43 mmol), NH₄Cl (15.43 g, 285.70 mmol), MeOH (200 mL) and H₂O (50 mL) at room temperature. The resulting mixture was stirred 2 h at 70° C. The resulting mixture was extracted with EtOAc (500 mL). The combined organic layers were washed with brine (2×400 mL), dried over anhydrous Na₂SO₄. After filtration, the filtrate was concentrated under reduced pressure. The residue was purified by silica gel column chromatography, eluted with PE/EtOAc (100:0-60:40) to afford 2-amino-3,6-difluorophenol (6.30 g, 76.03%) as yellow solid.

### Step 2: 2-amino-5-bromo-3,6-difluorophenol

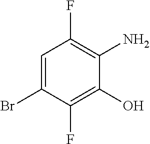 [SMILES: Nc1c(F)cc(Br)c(F)c1O]

Into a 500-mL round-bottom flask purged and maintained with N₂, were added 2-amino-3,6-difluorophenol (6.10 g, 42.07 mmol), NBS (8.24 g, 46.28 mmol) and DMF (150 mL) at 0° C. The resulting mixture was stirred 2 h at room temperature. The resulting mixture was extracted with EtOAc (500 mL). The combined organic layers were washed with brine (2×400 mL), dried over anhydrous Na₂SO₄. After filtration, the filtrate was concentrated under reduced pressure. The residue was purified by silica gel column chromatography, eluted with PE/EtOAc (100:0-60:40) to afford 2-amino-5-bromo-3,6-difluorophenol (3.10 g, 32.00%) as yellow solid.

### Step 3: N,N-dibenzyl-2-(benzyloxy)-4-bromo-3,6-difluoroaniline

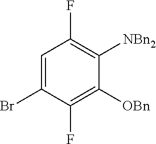

Into a 500-mL round-bottom flask purged and maintained with H₂, were added 2-amino-5-bromo-3,6-difluorophenol (3.10 g, 13.90 mmol), NaH (2.34 g, 58.39 mmol) and DMF (100 mL) at room temperature. The resulting mixture was stirred 0.5 h at room temperature. To a solution of BnBr (9.56 g, 55.60 mmol) at room temperature. The resulting mixture was stirred for 16 h at 80° C. The precipitated solids were collected by filtration and washed with EtOAc (500 mL). After filtration, the filtrate was concentrated under reduced pressure. The residue was purified by silica gel column chromatography, eluted with PE/EtOAc (100:0-90:10) to afford N,N-dibenzyl-2-(benzyloxy)-4-bromo-3,6-difluoroaniline (6.0 g, 87.55%) as yellow oil.

### Step 4: N,N-dibenzyl-2-(benzyloxy)-4-(4-(dimethoxymethyl)piperidin-1-yl)-3,6-difluoroaniline

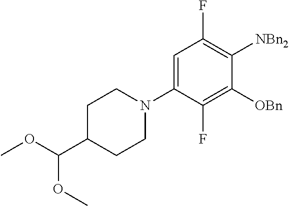

Into a 40-mL vial flask purged and maintained with N₂, were added N,N-dibenzyl-2-(benzyloxy)-4-bromo-3,6-difluoroaniline (0.90 g, 1.83 mmol), 4-(dimethoxymethyl)piperidine (0.58 g, 3.65 mmol), Pd-PEPPSI₂Me-IPENT Cl (76.9 mg, 0.09 mmol), Cs₂CO₃ (0.89 g, 2.75 mmol) and Dioxane (20 mL) at room temperature. The resulting mixture was stirred 2 h at 100° C. After filtration, the filtrate was concentrated under reduced pressure. The residue was purified by silica gel column chromatography, eluted with PE/EtOAc (100:0-80:20) to afford N,N-dibenzyl-2-(benzyloxy)-4-(4-(dimethoxymethyl)piperidin-1-yl)-3,6-difluoroaniline (0.77 g, 59.73%) as yellow solid.

### Step 5: 6-(4-(dimethoxymethyl)piperidin-1-yl)-4,7-difluorobenzo[d]oxazol-2(3H)-one

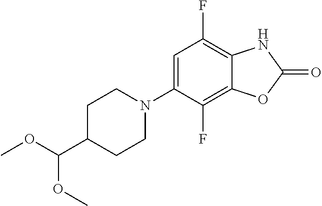 [SMILES: COC(OC)C1CCN(c2cc(F)c3[nH]c(=O)oc3c2F)CC1]

Into a 250-mL round-bottom flask purged and maintained with H₂, were added N,N-dibenzyl-2-(benzyloxy)-4-(4-(dimethoxymethyl)piperidin-1-yl)-3,6-difluoroaniline (2.30 g, 4.02 mmol), Pd/C (2.30 g) and THF (100 mL) at room temperature under H₂ atmosphere. The resulting mixture was stirred 15 h at room temperature. To a solution of CDI (1.30 g, 8.04 mmol) at room temperature. The resulting mixture was stirred for 3 h at room temperature. The precipitated solids were collected by filtration and washed with EtOAc (500 mL). After filtration, the filtrate was concentrated under reduced pressure. The residue was purified by silica gel column chromatography, eluted with PE/EtOAc (100:0-60:40) to afford 6-(4-(dimethoxymethyl)piperidin-1-yl)-4,7-difluorobenzo[d]oxazol-2(3H)-one (0.65 g, 49.28%) as yellow solid.

### Step 6: 3-(6-(4-(dimethoxymethyl)piperidin-1-yl)-4,7-difluoro-2-oxobenzo[d]oxazol-3(2H)-yl)piperidine-2,6-dione

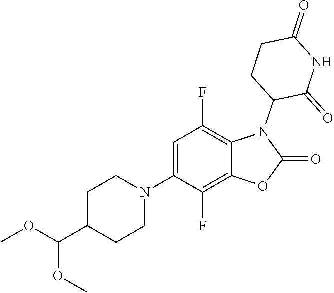 [SMILES: COC(OC)C1CCN(c2cc(F)c3c(oc(=O)n3C3CCC(=O)NC3=O)c2F)CC1]

Into a 40-mL Vial flask purged and maintained with N₂, were added 6-(4-(dimethoxymethyl)piperidin-1-yl)-4,7-difluorobenzo[d]oxazol-2(3H)-one (0.65 g, 1.98 mmol), 3-bromopiperidine-2,6-dione (0.76 g, 3.96 mmol), THF (20 mL) and NaH (0.24 g, 5.94 mmol) at 0° C. The resulting mixture was stirred 15 h at 60° C. The resulting mixture was extracted with EtOAc (200 mL). The combined organic layers were washed with brine (2×200 mL), dried over anhydrous Na₂SO₄. After filtration, the filtrate was concentrated under reduced pressure. The residue was purified by silica gel column chromatography, eluted with PE/EtOAc (100:0-70:30) to afford 3-(6-(4-(dimethoxymethyl)piperidin-1-yl)-4,7-difluoro-2-oxobenzo[d]oxazol-3(2H)-yl)piperidine-2,6-dione (215.4 mg, 24.14%) as light pink solid.

### Step 7: 1-(3-(2,6-dioxopiperidin-3-yl)-4,7-difluoro-2-oxo-2,3-dihydrobenzo[d]oxazol-6-yl)piperidine-4-carbaldehyde

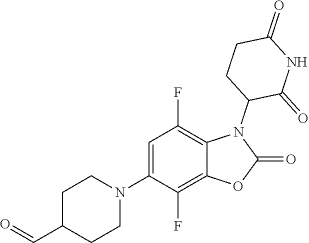 [SMILES: O=CC1CCN(c2cc(F)c3c(oc(=O)n3C3CCC(=O)NC3=O)c2F)CC1]

To a flask was added 3-(6-(4-(dimethoxymethyl)piperidin-1-yl)-4,7-difluoro-2-oxobenzo[d]oxazol-3(2H)-yl)piperidine-2,6-dione (100 mg, 0.2 mmol) and formic acid (5 mL). The mixture was stirred at room temperature for 2 h. The solution was concentrated under reduced pressure to give the crude product. LC-MS (ESI): m/z [M+H]⁺=394.4.

### Step 8: (1S,3R)-3-(3-((2-(1-((1-(3-((S)-2,6-dioxopiperidin-3-yl)-4,7-difluoro-2-oxo-2,3-dihydrobenzo[d]oxazol-6-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-(1-((1-(3-((R)-2,6-dioxopiperidin-3-yl)-4,7-difluoro-2-oxo-2,3-dihydrobenzo[d]oxazol-6-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

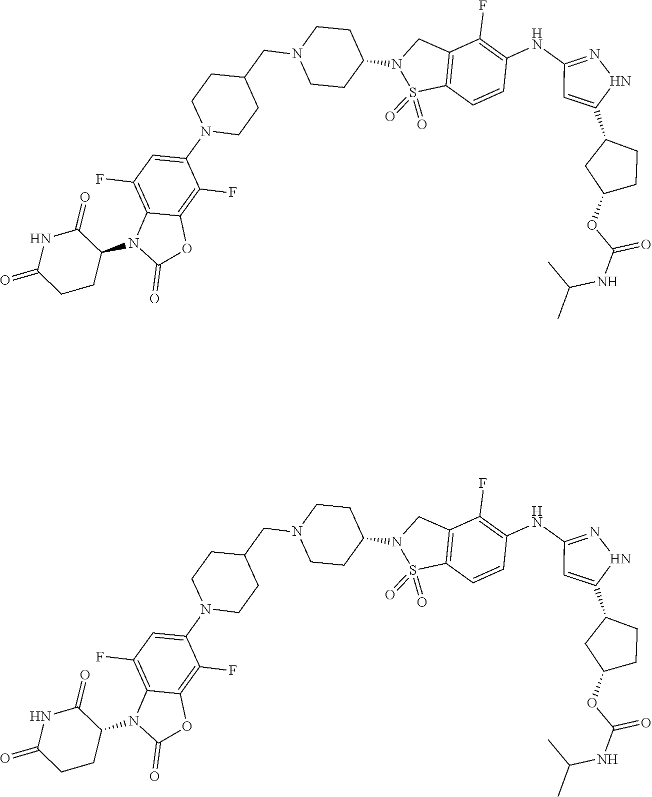

The titled compound was synthesized in the procedures similar to Example 280 in a racemate form, which was further separated by chiral HPLC to give:

Enantiomer 1 (Example 283, 100% ee); Retention time: 4.242 min. ¹H NMR (500 MHz, DMSO) δ 11.99 (s, 1H), 11.24 (s, 1H), 8.76 (s, 1H), 8.30 (s, 1H), 7.51 (d, J=9.1 Hz, 1H), 6.93 (s, 1H), 6.87 (d, J=12.7 Hz, 1H), 5.83 (s, 1H), 5.40 (s, 1H), 4.99 (s, 1H), 4.51 (s, 2H), 3.63-3.53 (m, 1H), 3.48-3.38 (m, 3H), 3.12-3.04 (m, 1H), 2.93-2.87 (m, 2H), 2.70-2.66 (s, 4H), 2.45-2.43 (m, 1H), 2.31-2.24 (m, 2H), 2.22-2.17 (m, 2H), 2.09-1.98 (m, 4H), 1.94-1.78 (m, 7H), 1.76-1.54 (m, 4H), 1.34-1.21 (m, 4H), 1.03 (d, J=6.0 Hz, 6H). LC-MS (ESI): m/z [M+H]⁺=898.4;

Enantiomer 2 (Example 282, 100% ee); Retention time: 3.286 min. ¹H NMR (500 MHz, DMSO) δ 11.99 (s, 1H), 11.24 (s, 1H), 8.76 (s, 1H), 8.30 (s, 1H), 7.51 (d, J=9.1 Hz, 1H), 6.93 (s, 1H), 6.87 (d, J=12.7 Hz, 1H), 5.83 (s, 1H), 5.40 (s, 1H), 4.99 (s, 1H), 4.51 (s, 2H), 3.63-3.53 (m, 1H), 3.48-3.38 (m, 3H), 3.12-3.04 (m, 1H), 2.93-2.87 (m, 2H), 2.70-2.66 (s, 4H), 2.45-2.43 (m, 1H), 2.31-2.24 (m, 2H), 2.22-2.17 (m, 2H), 2.09-1.98 (m, 4H), 1.94-1.78 (m, 7H), 1.76-1.54 (m, 4H), 1.34-1.21 (m, 4H), 1.03 (d, J=6.0 Hz, 6H). LC-MS (ESI): m/z [M+H]⁺=898.4;

Chiral analytical method: Column: ICHIRALPAK IH 4.6*150 mm 5 μm; Mobile phase: A for MtBE (0.1%2M NH₃ MeOH) (%) and B for MeOH:DCM=50:50(%); Gradient: Mobile Phase A: Mobile Phase B=70:30 (v/v); HPLC Equipment: HPLC-Agilent, Back pressure: 100 bar; Column temperature: 35° C.

Chiral Prep-HPLC Condition: CHIRALPAK IH 250*20.0 mml, 5 μm; Mobile phase: A for MtBE (0.1%2M NH3 MeOH) (%) and B for MeOH:DCM=50:50; Gradient: Mobile Phase A: Mobile Phase B=70:30 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 284 and 285: (1S,3R)-3-(3-((2-(1′-(3-((S)-2,6-dioxopiperidin-3-yl)-4,7-difluoro-2-oxo-2,3-dihydrobenzo[d]oxazol-6-yl)-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-(1′-(3-((R)-2,6-dioxopiperidin-3-yl)-4,7-difluoro-2-oxo-2,3-dihydrobenzo[d]oxazol-6-yl)-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

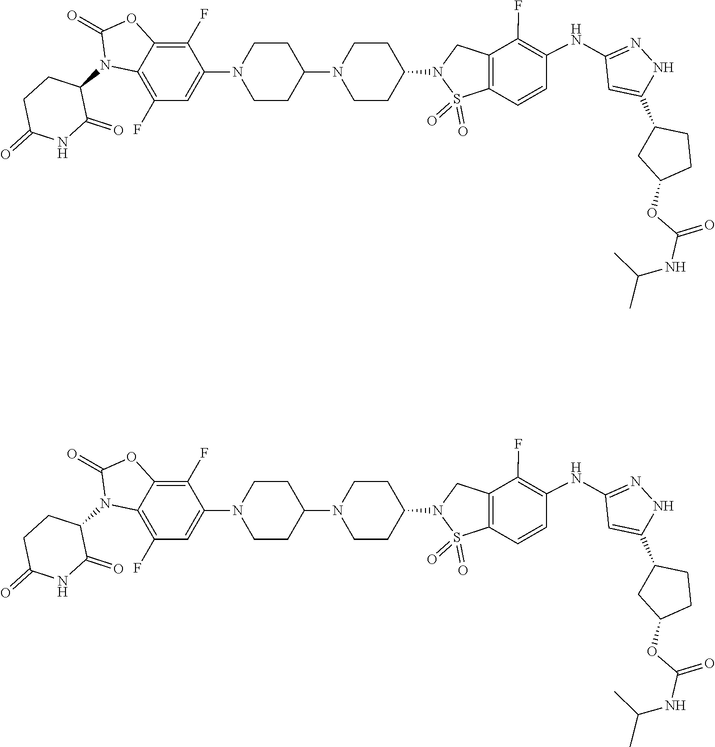

To a solution of 3-(4,7-difluoro-2-oxo-6-(4-oxopiperidin-1-yl)benzo[d]oxazol-3(2H)-yl)piperidine-2,6-dione (50.0 mg, 0.132 mmol) in DMA (3 mL), (1S,3R)-3-(3-((4-fluoro-1,1-dioxido-2-(piperidin-4-yl)-2,3-dihydrobenzo[d]isothiazol-5-yl)amino)-1H-pyrazol-5-yl)cyclopentyl isopropylcarbamate (69 mg, 0.132 mmol) were added at rt. The resulting mixture was stirred for 10 mins. Then sodium cyanoborohydride (25.0 mg, 0.396 mmol) and acetic acid (one drop) were added into the mixture and stirred for 2 h. The reaction was detected by LCMS, after the residue was purified by silica gel chromatography, eluting with DCM/MeOH (10:1; R_{f}=0.32) to afford the crude product (14.3 mg, 12.3% yield), which was further separated by Chiral Prep-HPLC to give:

Enantiomer 1 (Example 285, 99.18% ee); Retention time: 11.98 min ¹H NMR (500 MHz, DMSO-d₆) δ 12.00 (s, 1H), 11.25 (s, 1H), 8.76 (s, 1H), 8.29 (s, 1H), 7.51 (d, J=8.6 Hz, 1H), 7.02-6.77 (m, 2H), 5.83 (s, 1H), 5.40 (m, 1H), 4.99 (s, 1H), 4.51 (s, 2H), 3.15-3.00 (m, 5H), 2.64 (m, 5H), 2.45 (m, 1H), 2.31 (m, 3H), 2.0 (m, 1H), 1.91 (m, 3H), 1.84 (m, 4H), 1.71 (m, 2H), 1.61 (m, 3H), 1.24 (s, 3H), 1.04 (d, J=5.8 Hz, 6H). LC-MS (ESI): m/z [M+H]⁺=884.48.

Enantiomer 2 (Example 284, 96.96% ee); Retention time: 14.99 min. ¹H NMR (500 MHz, DMSO-d₆) δ 11.99 (s, 1H), 11.25 (s, 1H), 8.76 (s, 1H), 8.29 (s, 1H), 7.51 (d, J=8.6 Hz, 1H), 7.02-6.77 (m, 2H), 5.83 (s, 1H), 5.36 (m, 1H), 4.97 (s, 1H), 4.51 (s, 2H), 3.15-3.00 (m, 5H), 2.68 (m, 5H), 2.45 (m, 1H), 2.31 (m, 3H), 2.0 (m, 1H), 1.91 (m, 3H), 1.84 (m, 4H), 1.71 (m, 2H), 1.61 (m, 3H), 1.24 (s, 3H), 1.04 (d, J=5.8 Hz, 6H). LC-MS (ESI): m/z [M+H]⁺=884.51.

Chiral analytical method: Column: Lux®5 μm i-Cellulose-5; Mobile phase: A for MtBE (0.1% NH3·MeOH) and B for DCM:MeOH=1:1; Gradient: Mobile Phase A: Mobile Phase B=50:50 (V/V); HPLC Equipment: Agilent 1260 Infinity II (ARD_LC-808); Column temperature: RT.

Chiral Prep-HPLC Condition: Column: Lux®5 μm i-Cellulose-5; Mobile phase: A for MtBE (0.2% NH3·MeOH) and B for DCM:MeOH=1:1; Gradient: Mobile Phase A: Mobile Phase B=50:50 (V/V); HPLC Equipment: Prep-HPLC Gilson (ARD_LC-815); Column temperature: RT.

### Example 286: (1S,3R)-3-(3-((2-(1-((1-(3-(2,6-dioxopiperidin-3-yl)-7-methoxy-2-oxo-2,3-dihydrobenzo[d]oxazol-6-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropyl carbamate

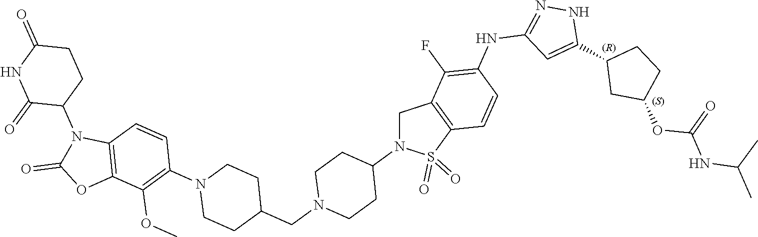

### Step 1: 2-amino-6-methoxyphenol

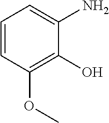

To a solution of 2-methoxy-6-nitrophenol (12 g, 71 mmol) in THF (200 mL) was added Pd/C (12 g, 10% wt). The resulting solution was stirred for 12 h at room temperature under H₂ atmosphere. After filtration, the filtrate was concentrated under vacuum. The residue was the product (7.1 g, 71.7%) as yellow solid. MS (ESI) m/e [M+1]⁺=140.

### Step 2: 7-methoxybenzo[d]oxazol-2(3H)-one

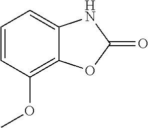

To a solution of 2-amino-6-methoxyphenol (7.1 g, 51.1 mmol) in MeCN (150 mL) was added CDI (12.4 g, 76.7 mmol). The resulting solution was stirred for 12 h at 70° C. under nitrogen atmosphere. The resulting mixture was concentrated under vacuum. The residue was purified by combi-flash (EtOAc/PE=22%) to give the product (8.2 g, 97.6%) as yellow solid. MS (ESI) m/e [M+1]⁺=166.

### Step 3: 7-methoxybenzo[d]oxazol-2(3H)-one

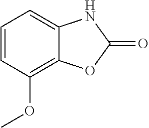

To a solution of 7-methoxybenzo[d]oxazol-2(3H)-one (8.2 g, 49.7 mmol) in DMF (100 mL) was added NBS (8.8 g, 49.7 mmol). The resulting solution was stirred for 5 h at room temperature. The resulting mixture was concentrated under vacuum. The residue was purified by combi-flash (EtOAc/PE=22%) to give the product (4 g, 33.1%) as yellow solid. MS (ESI) m/e [M+1]⁺=244/246.

### Step 4: 3-(2,6-bis(benzyloxy)pyridin-3-yl)-6-bromo-7-methoxybenzo[d]oxazol-2(3H)-one

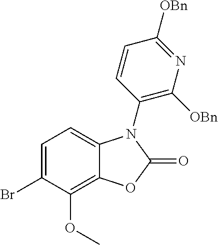 [SMILES: COc1c(Br)ccc2c1oc(=O)n2-c1ccc(OCc2ccccc2)nc1OCc1ccccc1]

To a solution of 6-bromo-7-methoxybenzo[d]oxazol-2(3H)-one (1 g, 4.1 mmol) in dioxane (20 mL) were added 2,6-bis(benzyloxy)-3-(4,4,5,5-tetramethyl-1,3,2-dioxaborolan-2-yl) pyridine (3.1 g, 7.4 mmol), Cu(OAc)₂ (1.1 g, 6.2 mmol), Pyridine (1.3 g, 16.4 mmol) and 4A MS (500 mg). The resulting solution was stirred for 12 h at 80° C. under oxygen atmosphere. After filtration, the filtrate was concentrated under vacuum. The residue was purified by combi-flash (EtOAc/PE=13%) to give the product (1 g, 45.5%) as red solid. MS (ESI) m/e [M+1]⁺=533/535.

### Step 5: 3-(2,6-bis(benzyloxy)pyridin-3-yl)-6-(4-(dimethoxymethyl) piperidin-1-yl)-7-methoxybenzo[d]oxazol-2(3H)-one

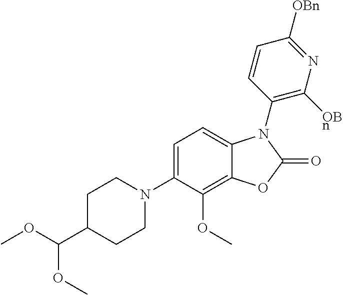 [SMILES: COc1c(N2CCC(C(OC)OC)CC2)ccc2c1oc(=O)n2-c1ccc(OCc2ccccc2)nc1OCc1ccccc1]

To a solution of 3-(2,6-bis(benzyloxy)pyridin-3-yl)-6-bromo-7-methoxybenzo[d]oxazol-2(3H)-one (942.2 mg, 1.8 mmol) in dioxane (20 mL) were added 4-(dimethoxymethyl)piperidine (394 mg, 2.5 mmol), Pd-PEPPSI-IPentCl 2-methylpyridine (o-picoline) (74.3 mg, 0.09 mmol) and Cs₂CO₃ (865.5 mg, 2.7 mmol). The resulting solution was stirred for 12 h at 100° C. under nitrogen atmosphere. The resulting mixture was concentrated under vacuum. The residue was purified by combi-flash (EtOAc/PE=28%) to give the product (300 mg, 27.3%) as yellow oil. MS (ESI) m/e [M+1]⁺=612.

### Step 6: 3-(6-(4-(dimethoxymethyl) piperidin-1-yl)-7-methoxy-2-oxobenzo[d]oxazol-3(2H)-yl)_piperidine-2,6-dione

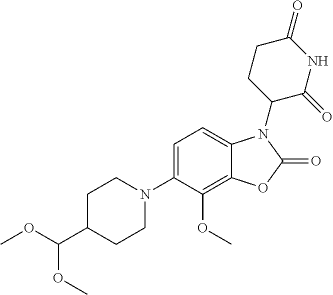 [SMILES: COc1c(N2CCC(C(OC)OC)CC2)ccc2c1oc(=O)n2C1CCC(=O)NC1=O]

To a solution of 3-(2,6-bis(benzyloxy)pyridin-3-yl)-6-(4-(dimethoxymethyl) piperidin-1-yl)-7-methoxybenzo[d]oxazol-2(3H)-one (300 mg, 0.5 mmol) in THF (5 mL) was added Pd/C (300 mg, 10% wt). The resulting solution was stirred for 12 h at 50° C. under H₂ atmosphere. After filtration, the filtrate was concentrated under vacuum. The residue was the product (218.1 mg, 100%) as light blue solid. MS (ESI) m/e [M+1]⁺=434.

### Step 7: 1-(3-(2,6-dioxopiperidin-3-yl)-7-methoxy-2-oxo-2,3-dihydrobenzo[d]oxazol-6-yl)piperidine-4-carbaldehyde

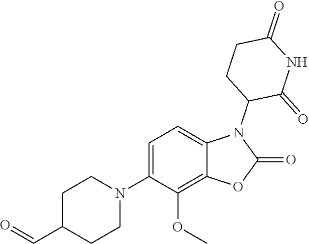 [SMILES: COc1c(N2CCC(C=O)CC2)ccc2c1oc(=O)n2C1CCC(=O)NC1=O]

To a flask was added 3-(6-(4-(dimethoxymethyl) piperidin-1-yl)-7-methoxy-2-oxobenzo[d]oxazol-3(2H)-yl) piperidine-2,6-dione (100 mg, 0.23 mmol) and formic acid (5 mL). The mixture was stirred at room temperature for 2 h. The solution was concentrated under reduced pressure to give the crude product. LC-MS (ESI): m/z [M+H]⁺=388.2

### Step 8: (1S,3R)-3-(3-((2-(1-((1-(3-(2,6-dioxopiperidin-3-yl)-7-methoxy-2-oxo-2,3-dihydrobenzo[d]oxazol-6-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropyl carbamate

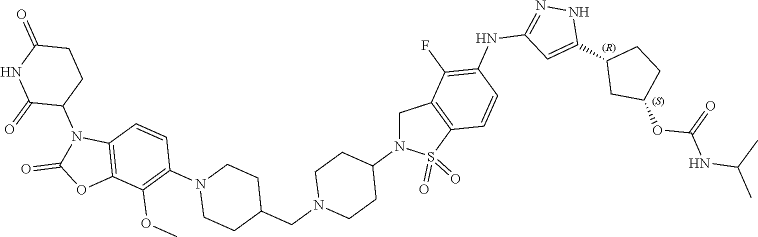

The titled compound was synthesized in the procedures similar to Example 62. ¹H NMR (500 MHz, DMSO) δ 12.01 (s, 1H), 11.20 (s, 1H), 8.80 (s, 1H), 8.31 (s, 1H), 7.54 (d, J=8.4 Hz, 1H), 6.94 (d, J=7.4 Hz, 1H), 6.82 (dd, J=23.2, 8.5 Hz, 2H), 5.84 (s, 1H), 5.31 (dd, J=12.9, 5.3 Hz, 1H), 5.00 (s, 1H), 4.55 (s, 2H), 4.00 (s, 3H), 3.84-3.36 (m, 4H), 3.30-3.23 (m, 2H), 3.20-3.02 (m, 3H), 2.92-2.83 (m, 1H), 2.75-2.54 (m, 8H), 2.22-2.11 (m, 3H), 2.06-1.99 (m, 2H), 1.95-1.82 (m, 4H), 1.76-1.59 (m, 3H), 1.40 (s, 2H), 1.07-0.98 (m, 6H). LC-MS (ESI): m/z [M+H]⁺=892.69.

### Example 287: (1S,3R)-3-(3-((2-(1′-(3-(2,6-dioxopiperidin-3-yl)-7-methoxy-2-oxo-2,3-dihydrobenzo[d]oxazol-6-yl)-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

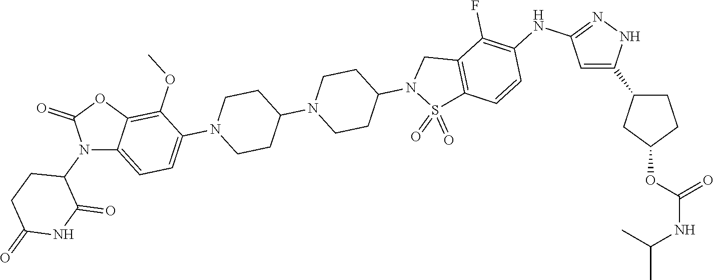 [SMILES: COc1c(N2CCC(N3CCC(N4Cc5c(ccc(Nc6cc([C@@H]7CC[C@H](OC(=O)NC(C)C)C7)[nH]n6)c5F)S4(=O)=O)CC3)CC2)ccc2c1oc(=O)n2C1CCC(=O)NC1=O]

### Step 1: 3-(2,6-bis(benzyloxy)pyridin-3-yl)-7-methoxy-6-(1,4-dioxa-8-azaspiro[4.5]decan-8-yl)benzo[d]oxazol-2(3H)-one

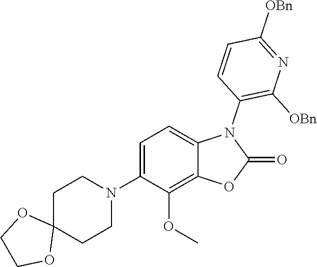 [SMILES: COc1c(N2CCC3(CC2)OCCO3)ccc2c1oc(=O)n2-c1ccc(OCc2ccccc2)nc1OCc1ccccc1]

To a solution of 3-(2,6-bis(benzyloxy)pyridin-3-yl)-6-bromo-7-methoxybenzo[d]oxazol-2(3H)-one (1.2 g, 2.3 mmol) in dioxane (40 mL) were added 4-(dimethoxymethyl)piperidine (483 mg, 3.4 mmol), Pd-PEPPSI-IPentCl 2-methylpyridine (96.6 mg, 0.115 mmol) and Cs₂CO₃ (1.5 g, 4.6 mmol). The resulting solution was stirred for 3 h at 100° C. under nitrogen atmosphere. The resulting mixture was concentrated under vacuum. The residue was purified by combi-flash (EtOAc/PE=80%) to give the product (540 mg, 40.3%) as yellow solid. LCMS (ESI) m/e [M+1]⁺=596.

### Step 2: 3-(7-methoxy-2-oxo-6-(1,4-dioxa-8-azaspiro[4.5]decan-8-yl)benzo[d]oxazol-3(2H)-yl)piperidine-2,6-dione

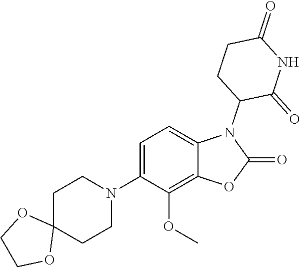 [SMILES: COc1c(N2CCC3(CC2)OCCO3)ccc2c1oc(=O)n2C1CCC(=O)NC1=O]

To a solution of 3-(2,6-bis(benzyloxy)pyridin-3-yl)-7-methoxy-6-(1,4-dioxa-8-azaspiro[4.5]decan-8-yl)benzo[d]oxazol-2(3H)-one (540 mg, 0.9 mmol) in THF (30 mL) was added Pd/C (540 mg, 10% wt). The resulting solution was stirred for 12 h at 50° C. under H₂ atmosphere. After filtration, the filtrate was concentrated under vacuum. The residue was the product (350 mg, 92.4%) as white solid. LCMS (ESI) m/e [M+1]⁺=418.

### Step 3: 3-(7-methoxy-2-oxo-6-(4-oxopiperidin-1-yl)benzo[d]oxazol-3(2H)-yl)piperidine-2,6-dione

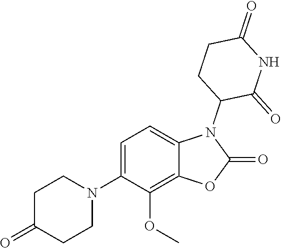 [SMILES: COc1c(N2CCC(=O)CC2)ccc2c1oc(=O)n2C1CCC(=O)NC1=O]

The solution of 3-(7-methoxy-2-oxo-6-(1,4-dioxa-8-azaspiro[4.5]decan-8-yl)benzo[d]oxazol-3(2H)-yl)piperidine-2,6-dione (330 mg, 0.8 mmol) in H₂SO₄ (20 mL) at 0° C. The resulting solution was stirred for 3 h at rt. The residue was basified to pH 5 with Na₂CO₃. The resulting mixture was extracted with EtOAc (3×20 mL). The combined organic layers were washed with brine (3×20 mL), dried over anhydrous Na₂SO₄. After filtration, the filtrate was concentrated under reduced pressure. The residue was the product (203.5 mg, 68.9%) as yellow solid. LCMS (ESI) m/e [M+1]⁺=374.

### Step 4: (1S,3R)-3-(3-((2-(1′-(3-(2,6-dioxopiperidin-3-yl)-7-methoxy-2-oxo-2,3-dihydrobenzo[d]oxazol-6-yl)-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

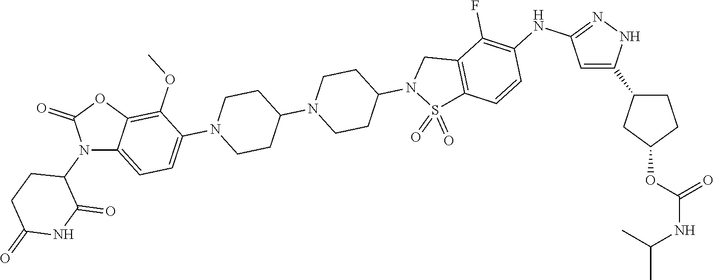 [SMILES: COc1c(N2CCC(N3CCC(N4Cc5c(ccc(Nc6cc([C@@H]7CC[C@H](OC(=O)NC(C)C)C7)[nH]n6)c5F)S4(=O)=O)CC3)CC2)ccc2c1oc(=O)n2C1CCC(=O)NC1=O]

The titled compound was synthesized in the procedures similar to Example 63. ¹H NMR (500 MHz, DMSO) δ 12.00 (s, 1H), 11.19 (s, 1H), 8.76 (s, 1H), 8.29 (s, 1H), 7.52 (s, 1H), 6.94 (s, 1H), 6.81 (dd, J=19.9, 8.6 Hz, 2H), 5.83 (s, 1H), 5.30 (dd, J=13.1, 5.2 Hz, 1H), 5.06-4.96 (m, 1H), 4.52 (s, 2H), 4.00 (s, 3H), 3.67-3.50 (m, 1H), 3.47-3.40 (m, 3H), 3.37-3.34 (m, 3H), 3.03 (d, J=38.0 Hz, 4H), 3.02-2.75 (m, 3H), 2.33-2.30 (m, 1H), 2.18-2.11 (m, 1H), 2.06-2.00 (m, 1H), 1.98-1.80 (m, 8H), 1.79-1.69 (m, 2H), 1.67-1.56 (m, 3H), 1.03 (d, J=6.0 Hz, 6H). LC-MS (ESI): m/z [M+H]⁺=878.4;

### Example 288 and 289: (1S,3R)-3-(3-((2-(1-((1-(3-((S)-2,6-dioxopiperidin-3-yl)-2-oxo-2,3-dihydrobenzo[d]oxazol-6-yl)piperidin-4-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-(1-((1-(3-((R)-2,6-dioxopiperidin-3-yl)-2-oxo-2,3-dihydrobenzo[d]oxazol-6-yl)piperidin-4-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl isopropylcarbamate

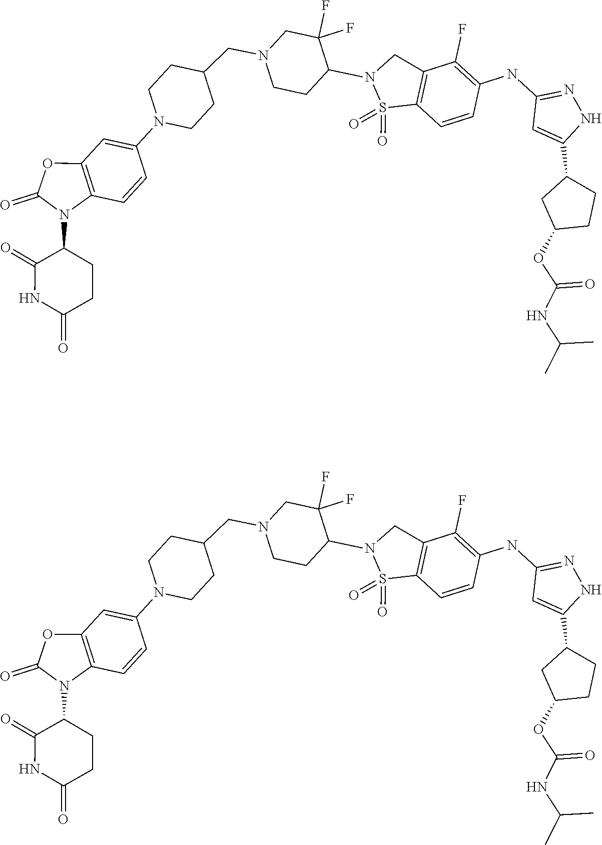

The titled compound was synthesized in the procedures similar to Example 62 in a racemic form, which was further separated by chiral HPLC to give:

Enantiomer 1 (Example 288, 100% ee); Retention time: 7.721 min. ¹H NMR (500 MHz, DMSO) δ 12.01 (s, 1H), 11.17 (s, 1H), 8.82 (s, 1H), 8.33 (t, J=7.7 Hz, 1H), 7.58 (d, J=8.6 Hz, 1H), 7.08 (s, 1H), 6.94 (d, J=7.6 Hz, 1H), 6.81-6.65 (m, 1H), 5.84 (s, 1H), 5.29 (dd, J=12.7, 5.3 Hz, 1H), 5.06-4.96 (m, 1H) 4.75 (d, J=14.5 Hz, 1H), 4.54 (d, J=13.3 Hz, 1H), 4.06-3.92 (m, 1H), 3.61 (d, J=11.4 Hz, 2H), 3.22-3.12 (m, 1H), 3.10-3.02 (m, 1H), 2.98-2.84 (m, 3H), 2.66-2.63 (m, 3H), 2.44-2.41 (m, 3H), 2.34-2.27 (m, 3H), 2.24-2.10 (m, 2H), 2.08-1.98 (m, 1H), 1.95-1.86 (m, 1H), 1.85-1.53 (m, 8H), 1.21 (s, 12H). LC-MS (ESI): m/z [M+H]⁺=898.4;

Enantiomer 1 (Example 289, 100% ee); Retention time: 9.807 min. ¹H NMR (500 MHz, DMSO) δ 12.01 (s, 1H), 11.17 (s, 1H), 8.82 (s, 1H), 8.33 (t, J=7.7 Hz, 1H), 7.58 (d, J=8.6 Hz, 1H), 7.08 (s, 1H), 6.94 (d, J=7.6 Hz, 1H), 6.81-6.65 (m, 1H), 5.84 (s, 1H), 5.29 (dd, J=12.7, 5.3 Hz, 1H), 5.06-4.96 (m, 1H) 4.75 (d, J=14.5 Hz, 1H), 4.54 (d, J=13.3 Hz, 1H), 4.06-3.92 (m, 1H), 3.61 (d, J=11.4 Hz, 2H), 3.22-3.12 (m, 1H), 3.10-3.02 (m, 1H), 2.98-2.84 (m, 3H), 2.66-2.63 (m, 3H), 2.44-2.41 (m, 3H), 2.34-2.27 (m, 3H), 2.24-2.10 (m, 2H), 2.08-1.98 (m, 1H), 1.95-1.86 (m, 1H), 1.85-1.53 (m, 8H), 1.21 (s, 12H). LC-MS (ESI): m/z [M+H]⁺=898.4;

Chiral analytical method: Column: ICHIRALPAK IC-5 4.6*150 mm 5 μm; Mobile phase: A for MtBE (0.1%2M NH3 MeOH) (%) and B for MeOH:DCM=50:50(%); Gradient: Mobile Phase A: Mobile Phase B=50:50 (v/v); HPLC Equipment: HPLC-Agilent, Back pressure: 100 bar; Column temperature: 35° C.

Chiral Prep-HPLC Condition: CHIRALPAK IC-5 250*20.0 mml, 5 μm; Mobile phase: A for MtBE (0.1%2M NH3 MeOH) (%) and B for MeOH:DCM=50:50; Gradient: Mobile Phase A: Mobile Phase B=50:50 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 290a and 290b: (1S,3R)-3-(3-((2-((4S)-1-((1-(3-(2,6-dioxopiperidin-3-yl)-2-oxo-2,3-dihydrobenzo[d]oxazol-7-yl)piperidin-4-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-((4R)-1-((1-(3-(2,6-dioxopiperidin-3-yl)-2-oxo-2,3-dihydrobenzo[d]oxazol-7-yl)piperidin-4-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl isopropylcarbamate

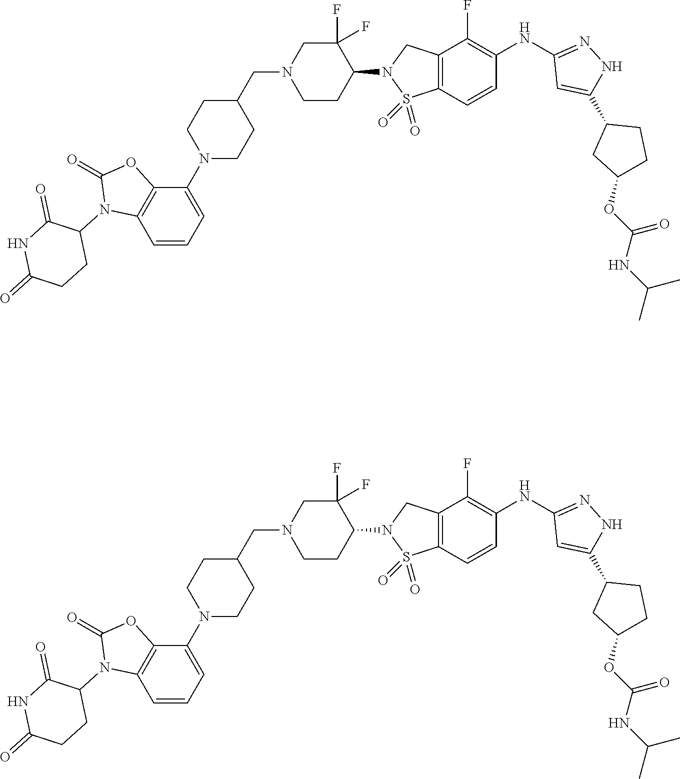 [SMILES: CC(C)NC(=O)O[C@H]1CC[C@@H](c2cc(Nc3ccc4c(c3F)CN([C@H]3CCN(CC5CCN(c6cccc7c6oc(=O)n7C6CCC(=O)NC6=O)CC5)CC3(F)F)S4(=O)=O)n[nH]2)C1 ; CC(C)NC(=O)O[C@H]1CC[C@@H](c2cc(Nc3ccc4c(c3F)CN([C@@H]3CCN(CC5CCN(c6cccc7c6oc(=O)n7C6CCC(=O)NC6=O)CC5)CC3(F)F)S4(=O)=O)n[nH]2)C1]

### Step 1: 1-(3-(2,6-dioxopiperidin-3-yl)-2-oxo-2,3-dihydrobenzo[d]oxazol-7-yl)piperidine-4-carbaldehyde

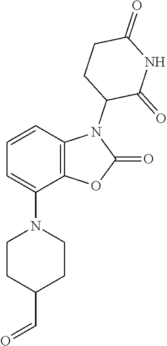 [SMILES: O=CC1CCN(c2cccc3c2oc(=O)n3C2CCC(=O)NC2=O)CC1]

The titled compound was synthesized in the procedures similar to Example 62. LC-MS (ESI): m/z [M+H]⁺=358.4

### Step 2: (1S,3R)-3-(3-((2-((4S)-1-((1-(3-(2,6-dioxopiperidin-3-yl)-2-oxo-2,3-dihydrobenzo[d]oxazol-7-yl)piperidin-4-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-((4R)-1-((1-(3-(2,6-dioxopiperidin-3-yl)-2-oxo-2,3-dihydrobenzo[d]oxazol-7-yl)piperidin-4-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl isopropylcarbamate

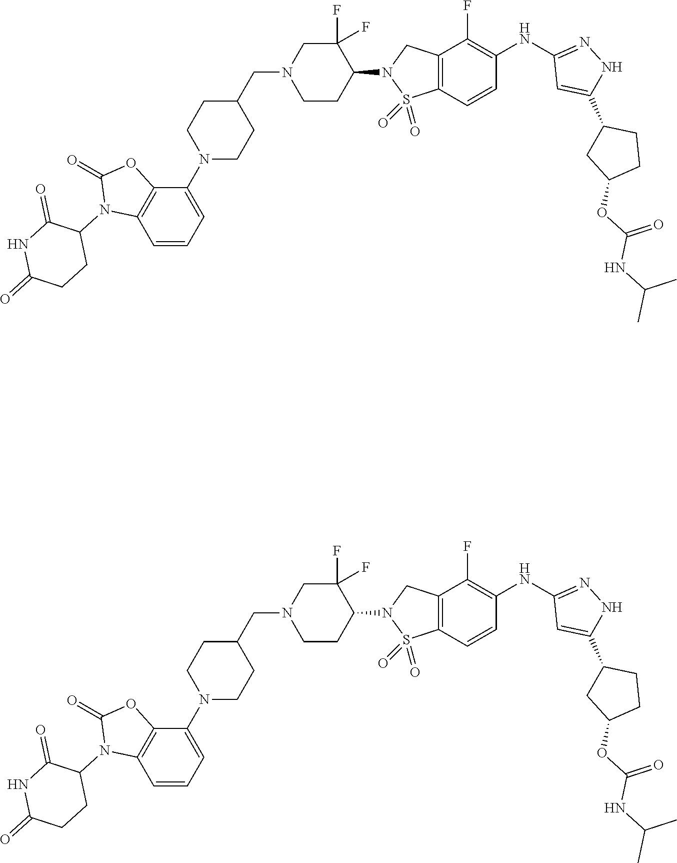 [SMILES: CC(C)NC(=O)O[C@H]1CC[C@@H](c2cc(Nc3ccc4c(c3F)CN([C@H]3CCN(CC5CCN(c6cccc7c6oc(=O)n7C6CCC(=O)NC6=O)CC5)CC3(F)F)S4(=O)=O)n[nH]2)C1 ; CC(C)NC(=O)O[C@H]1CC[C@@H](c2cc(Nc3ccc4c(c3F)CN([C@@H]3CCN(CC5CCN(c6cccc7c6oc(=O)n7C6CCC(=O)NC6=O)CC5)CC3(F)F)S4(=O)=O)n[nH]2)C1]

The titled compound was synthesized in the procedures similar to Example 62 in a racemic form, which was further separated with chiral HPLC to give:

Enantiomer 1 (Example 290a, 100% ee); Retention time: 5.946, 6.781 min. ¹H NMR (500 MHz, DMSO) δ 12.00 (s, 1H), 11.19 (s, 1H), 8.82 (s, 1H), 7.58 (d, J=8.7 Hz, 1H), 7.08 (t, J=8.1 Hz, 1H), 6.94 (d, J=7.4 Hz, 1H), 6.76 (d, J=7.9 Hz, 1H), 6.72 (d, J=8.5 Hz, 1H), 5.84 (s, 1H), 5.32 (dd, J=12.9, 5.2 Hz, 1H), 5.06-4.96 (m, 1H), 4.76 (d, J=14.4 Hz, 1H), 4.54 (d, J=13.7 Hz, 1H), 4.06-3.92 (m, 1H), 3.72-3.64 (m, 2H), 3.60-3.54 (m, 1H), 3.22-3.12 (m, 2H), 3.10-3.00 (m, 1H), 3.00-2.84 (m, 2H), 2.82-2.72 (m, 2H), 2.72-2.62 (m, 3H), 2.60-2.54 (m, 1H), 2.34-2.28 (m, 4H), 2.26-2.10 (m, 2H), 2.08-1.98 (m, 1H), 1.96-1.88 (m, 1H), 1.87-1.78 (m, 3H), 1.76-1.66 (m, 3H), 1.64-1.54 (m, 1H), 1.34-1.22 (m, 2H), 1.03 (d, J=6.2 Hz, 6H). LC-MS (ESI): m/z [M+H]⁺=898.4;

Enantiomer 2 (Example 290b, 100% ee); Retention time: 12.718, 13.015 min. ¹H NMR (500 MHz, DMSO) δ 12.00 (s, 1H), 11.19 (s, 1H), 8.82 (s, 1H), 7.58 (d, J=8.7 Hz, 1H), 7.08 (t, J=8.1 Hz, 1H), 6.94 (d, J=7.4 Hz, 1H), 6.76 (d, J=7.9 Hz, 1H), 6.72 (d, J=8.5 Hz, 1H), 5.84 (s, 1H), 5.32 (dd, J=12.9, 5.2 Hz, 1H), 5.06-4.96 (m, 1H), 4.76 (d, J=14.4 Hz, 1H), 4.54 (d, J=13.7 Hz, 1H), 4.06-3.92 (m, 1H), 3.72-3.64 (m, 2H), 3.60-3.54 (m, 1H), 3.22-3.12 (m, 2H), 3.10-3.00 (m, 1H), 3.00-2.84 (m, 2H), 2.82-2.72 (m, 2H), 2.72-2.62 (m, 3H), 2.60-2.54 (m, 1H), 2.34-2.28 (m, 4H), 2.26-2.10 (m, 2H), 2.08-1.98 (m, 1H), 1.96-1.88 (m, 1H), 1.87-1.78 (m, 3H), 1.76-1.66 (m, 3H), 1.64-1.54 (m, 1H), 1.34-1.22 (m, 2H), 1.03 (d, J=6.2 Hz, 6H). LC-MS (ESI): m/z [M+H]⁺=898.4;

Chiral analytical method: Column: ICHIRALPAK IE 4.6*150 mm 5 μm; Mobile phase: A for MtBE (0.1%2M NH3 MeOH) (%) and B for MeOH:DCM=50:50(%); Gradient: Mobile Phase A: Mobile Phase B=20:80 (v/v); HPLC Equipment: HPLC-Agilent, Back pressure: 100 bar; Column temperature: 35° C.

Chiral Prep-HPLC Condition: CHIRALPAK IE 250*20.0 mml, 5 μm; Mobile phase: A for MtBE (0.1%2M NH3 MeOH) (%) and B for MeOH:DCM=50:50; Gradient: Mobile Phase A: Mobile Phase B=20:80 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 291: (1S,3R)-3-(3-((2-(1-(1-(3-(2,6-dioxopiperidin-3-yl)-2-oxo-2,3-dihydrobenzo[d]oxazol-6-yl)azetidin-3-yl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

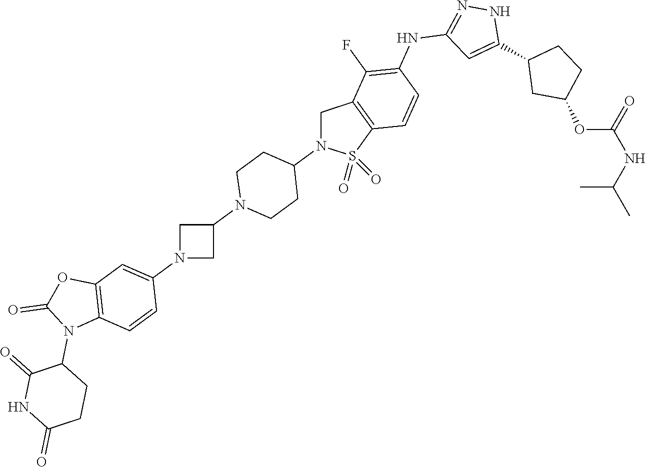 [SMILES: CC(C)NC(=O)O[C@H]1CC[C@@H](c2cc(Nc3ccc4c(c3F)CN(C3CCN(C5CN(c6ccc7c(c6)oc(=O)n7C6CCC(=O)NC6=O)C5)CC3)S4(=O)=O)n[nH]2)C1]

The titled compound was synthesized in the procedures similar to Example 101. ¹H NMR (500 MHz, DMSO-d₆) δ 12.00 (s, 1H), 11.17 (s, 1H), 8.77 (s, 1H), 8.28 (s, 1H), 7.51 (d, J=10 Hz, 1H), 7.05-6.93 (m, 1H), 6.76-6.69 (m, 1H), 6.38-6.31 (m, 1H), 5.83 (s, 1H), 5.34-5.25 (m, 1H), 5.06-4.96 (m, 1H), 4.51 (s, 2H), 3.99-2.87 (m, 2H), 3.63-3.42 (m, 4H), 3.28-3.22 (m, 1H), 3.11-3.01 (m, 1H), 2.94-2.79 (m, 3H), 2.72-2.60 (m, 2H), 2.47-2.43 (m, 1H), 2.16-2.09 (m, 1H), 2.08-1.80 (m, 7H), 1.78-1.65 (m, 2H), 1.64-1.45 (m, 1H), 1.09-0.97 (m, 6H). LC-MS (ESI): m/z [M+H]⁺=820.

### Example 292: (1S,3R)-3-(3-((2-(1-(1-(3-(2,6-dioxopiperidin-3-yl)-2-oxo-2,3-dihydrobenzo[d]oxazol-7-yl)azetidin-3-yl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

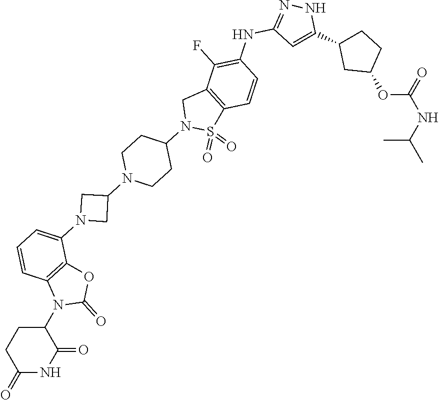 [SMILES: CC(C)NC(=O)O[C@H]1CC[C@@H](c2cc(Nc3ccc4c(c3F)CN(C3CCN(C5CN(c6cccc7c6oc(=O)n7C6CCC(=O)NC6=O)C5)CC3)S4(=O)=O)n[nH]2)C1]

### Step 1: 3-(2-oxo-7-(3-oxoazetidin-1-yl)benzo[d]oxazol-3(2H)-yl)piperidine-2,6-dione

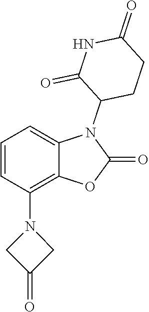 [SMILES: O=C1CN(c2cccc3c2oc(=O)n3C2CCC(=O)NC2=O)C1]

The titled compound was synthesized in the procedures similar to Example 101. LC-MS (ESI): m/z [M+H]⁺=316.2

### Step 2: (1S,3R)-3-(3-((2-(1-(1-(3-(2,6-dioxopiperidin-3-yl)-2-oxo-2,3-dihydrobenzo[d]oxazol-7-yl)azetidin-3-yl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

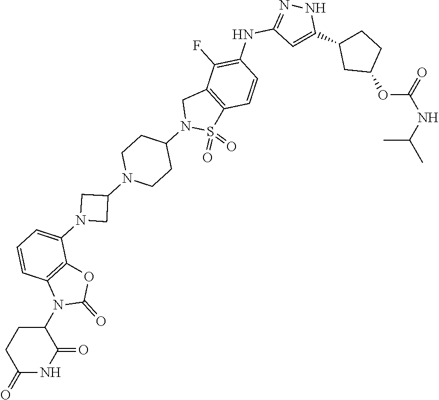 [SMILES: CC(C)NC(=O)O[C@H]1CC[C@@H](c2cc(Nc3ccc4c(c3F)CN(C3CCN(C5CN(c6cccc7c6oc(=O)n7C6CCC(=O)NC6=O)C5)CC3)S4(=O)=O)n[nH]2)C1]

The titled compound was synthesized in the procedures similar to Example 101. ¹H NMR (500 MHz, DMSO-d₆) δ 11.99 (s, 1H), 11.19 (s, 1H), 8.76 (s, 1H), 8.29 (s, 1H), 7.51 (d, J=10 Hz, 1H), 7.08-6.99 (m, 1H), 6.98-6.89 (m, 1H), 6.59 (d, J=10 Hz, 1H), 6.27 (d, J=10 Hz, 1H), 5.83 (s, 1H), 5.35-5.25 (m, 1H), 5.05-4.93 (m, 1H), 4.51 (s, 2H), 4.18-4.05 (m, 2H), 3.83-3.74 (m, 2H), 3.63-3.42 (m, 2H), 3.13-3.01 (m, 1H), 2.95-2.81 (m, 3H), 2.75-2.60 (m, 3H), 2.47-2.43 (m, 1H), 2.21-2.09 (m, 1H), 2.08-1.80 (m, 8H), 1.77-1.65 (m, 2H), 1.64-1.45 (m, 1H), 1.09-0.97 (m, 6H). LC-MS (ESI): m/z [M+H]⁺=820.

### Example 293: (1S,3R)-3-(3-((2-(1-((1-(3-(2,6-dioxopiperidin-3-yl)-2-oxo-2,3-dihydrobenzo[d]oxazol-6-yl)azetidin-3-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

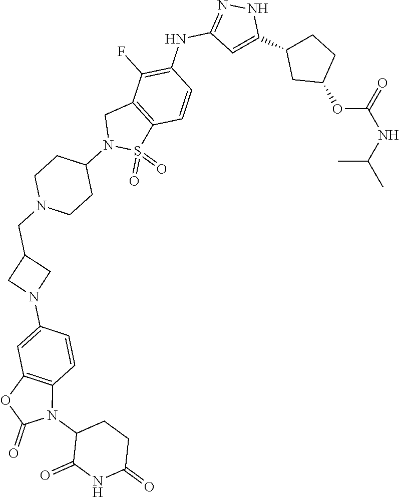 [SMILES: CC(C)NC(=O)O[C@H]1CC[C@@H](c2cc(Nc3ccc4c(c3F)CN(C3CCN(CC5CN(c6ccc7c(c6)oc(=O)n7C6CCC(=O)NC6=O)C5)CC3)S4(=O)=O)n[nH]2)C1]

The titled compound was synthesized in the procedures similar to Example 101. ¹H NMR (500 MHz, DMSO-d₆) δ 11.99 (s, 1H), 11.16 (s, 1H), 8.76 (s, 1H), 8.29 (s, 1H), 7.51 (d, J=5 Hz, 1H), 7.08-7.01 (m, 1H), 6.97-6.91 (m, 1H), 6.53 (s, 1H), 6.26-6.19 (m, 1H), 5.83 (s, 1H), 5.31-5.20 (m, 1H), 5.05-4.95 (m, 1H), 4.51 (s, 2H), 3.95-3.85 (m, 2H), 3.63-3.51 (m, 1H), 3.47-3.39 (m, 2H), 3.13-3.01 (m, 1H), 2.95-2.81 (m, 4H), 2.72-2.55 (m, 5H), 2.47-2.43 (m, 1H), 2.17-1.97 (m, 4H), 1.96-1.79 (m, 5H), 1.77-1.65 (m, 2H), 1.64-1.45 (m, 1H), 1.09-0.97 (m, 6H). LC-MS (ESI): m/z [M+H]⁺=834.

### Example 294a and 294b: (1S,3R)-3-(3-((2-((S)-1-((1-(3-((S)-2,6-dioxopiperidin-3-yl)-2-oxo-2,3-dihydrobenzo[d]oxazol-6-yl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropyl carbamate and (1S,3R)-3-(3-((2-((R)-1-((1-(3-((R)-2,6-dioxopiperidin-3-yl)-2-oxo-2,3-dihydrobenzo[d]oxazol-6-yl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropyl carbamate

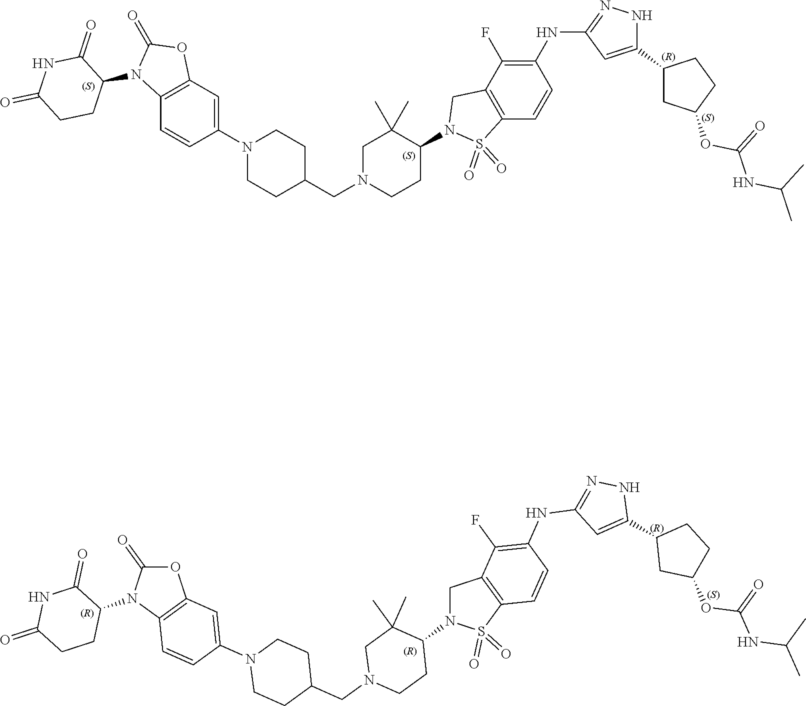

The titled compound was synthesized in the procedures similar to Example 6 in a racemic form, which was further separated by chiral HPLC to give:

Enantiomer 1 (Example 294a, 100% ee); Retention time: 5.029 min. ¹H NMR (500 MHz, DMSO) δ 11.99 (s, 1H), 11.17 (s, 1H), 8.77 (s, 1H), 8.31 (t, 1H), 7.54 (d, J=8.6 Hz, 1H), 7.07 (d, J=9.1 Hz, 2H), 6.93 (d, 1H), 6.78 (d, J=8.3 Hz, 1H), 5.83 (s, 1H), 5.29 (q, J=12.7 Hz, 1H), 5.00 (s, 1H), 4.54 (s, 2H), 3.60 (s, 3H), 3.23-3.17 (m, 3H), 3.06 (t, 1H), 2.95-2.88 (m, 2H), 2.66-2.62 (m, 4H), 2.19-2.09 (m, 4H), 2.05-1.91 (m, 3H), 1.81-1.71 (m, 4H), 1.62 (s, 2H), 1.50 (d, J=9.0 Hz, 1H), 1.27-1.20 (m, 3H), 1.12 (s, 3H), 1.03 (d, J=5.8 Hz, 6H), 0.93 (s, 3H). LC-MS (ESI): m/z [M+H]⁺=890.44.

Enantiomer 2 (Example 294b, 100% ee); Retention time: 9.95 min. ¹H NMR (500 MHz, DMSO) δ 11.99 (s, 1H), 11.17 (s, 1H), 8.77 (s, 1H), 8.31 (t, 1H), 7.54 (d, J=8.6 Hz, 1H), 7.07 (d, J=9.1 Hz, 2H), 6.93 (d, 1H), 6.78 (d, J=8.3 Hz, 1H), 5.83 (s, 1H), 5.29 (q, J=12.7 Hz, 1H), 5.00 (s, 1H), 4.54 (s, 2H), 3.60 (s, 3H), 3.23-3.17 (m, 3H), 3.06 (t, 1H), 2.95-2.88 (m, 2H), 2.66-2.62 (m, 4H), 2.19-2.09 (m, 4H), 2.05-1.91 (m, 3H), 1.81-1.71 (m, 4H), 1.62 (s, 2H), 1.50 (d, J=9.0 Hz, 1H), 1.27-1.20 (m, 3H), 1.12 (s, 3H), 1.03 (d, J=5.8 Hz, 6H), 0.93 (s, 3H). LC-MS (ESI): m/z [M+H]⁺=890.42.

Chiral analytical method: Column: YMC Amylose-SA 4.6*250 mm 5 μm Mobile phase: A for MtBE (0.1%2M NH3 MeOH) (%) and B for MeOH:DCM=50:50 (v/v); Gradient: Mobile Phase A: Mobile Phase B=30:70 (v/v); HPLC Equipment: Agilent 1260 Infinity II (ARD_LC-808); Column temperature: 25° C.

Chiral Prep-HPLC Condition: YMC Amylose-SA 4.6*250 mm 5 μm Mobile phase: A for MtBE (0.1%2M NH3 MeOH)(%) and B for MeOH:DCM=50:50; Gradient: Mobile Phase A: Mobile Phase B=30:70 (v/v); Flow Rate: 1 mL/min, Wavelength: UV 250 nm and 280 nm, Prep-HPLC Equipment: Prep-HPLC Gilson (ARD_LC-808); Column temperature: 25° C.

### Example 295 and 296: (1S,3R)-3-(3-((2-((R)-1′-(3-((S)-2,6-dioxopiperidin-3-yl)-2-oxo-2,3-dihydrobenzo[d]oxazol-7-yl)-3,3-dimethyl-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropyl carbamate and (1S,3R)-3-(3-((2-((R)-1′-(3-((R)-2,6-dioxopiperidin-3-yl)-2-oxo-2,3-dihydrobenzo[d]oxazol-7-yl)-3,3-dimethyl-[1,4′-bipiperidin]-4-yl)-4-fluoro-1,1-dioxido-2,3-dihydrobenzo[d]isoth-iazol-5-yl)amino)-1H-pyrazol-5-yl)cyclopentyl isopropyl carbamate

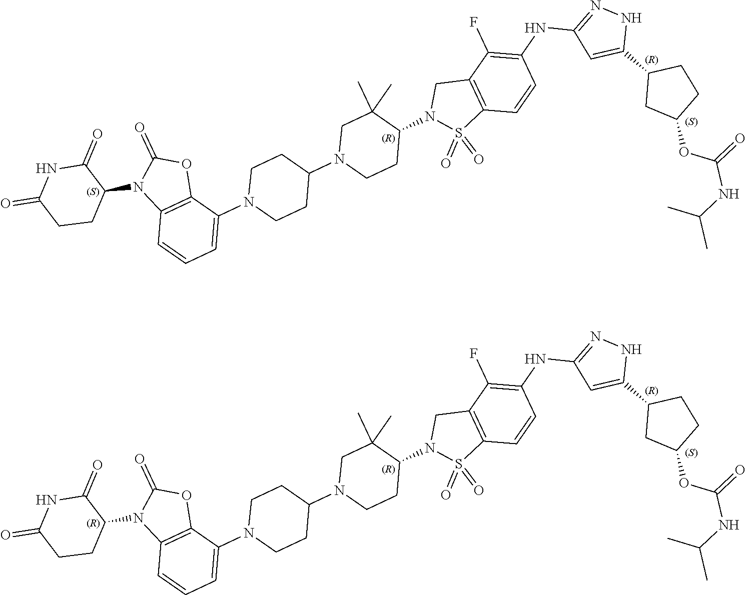

### Step 1: 3-(2,6-bis(benzyloxy)pyridin-3-yl)-7-(1,4-dioxa-8-azaspiro[4.5]decan-8-yl)benzo[d]oxazol-2(3H)-one

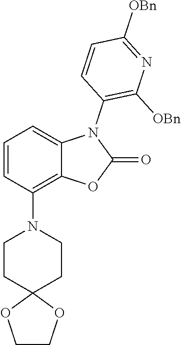 [SMILES: O=c1oc2c(N3CCC4(CC3)OCCO4)cccc2n1-c1ccc(OCc2ccccc2)nc1OCc1ccccc1]

To a solution of 3-(2,6-bis(benzyloxy)pyridin-3-yl)-7-bromobenzo[d]oxazol-2(3H)-one(PH-BGY-H-A517-11) (2.5 g, 4.98 mmol) in Dioxane (100 mL) were added 1,4-dioxa-8-azaspiro[4.5]decane (1.07 g, 7.47 mmol), Pd-PEPPSI-IpentCl (209 mg, 0.25 mmol) and Cs₂CO₃ (4.87 g, 14.94 mmol) at 100° C. for overnight under N₂ atmosphere. The reaction was concentrated under reduced pressure. The crude was purified by combine-flash PE:EA=100:0 to 65:35 to give the product (1.9 g, 67.6%) as light yellow solid.

### Step 2: Synthesis of 3-(2-oxo-7-(1,4-dioxa-8-azaspiro[4.5]decan-8-yl)benzo[d]oxazol-3(2H)-yl)piperidine-2,6-dione

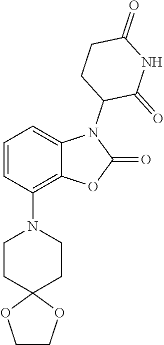 [SMILES: O=C1CCC(n2c(=O)oc3c(N4CCC5(CC4)OCCO5)cccc32)C(=O)N1]

To a solution of 3-(2,6-bis(benzyloxy)pyridin-3-yl)-7-(1,4-dioxa-8-azaspiro[4.5]decan-8-yl)benzo[d]oxazol-2(3H)-one (1.9 g, 3.36 mmol) in THF (100 mL) was added Pd/C (1.9 g) at 50° C. for overnight under H₂ atmosphere. The reaction was filtered. The filtrate was concentrated under vacuum. The residue was without purified to give the product (1 g, 76.9%) as yellow semi-solid.

### Step 3: 3-(2-oxo-7-(4-oxopiperidin-1-yl)benzo[d]oxazol-3(2H)-yl)piperidine-2,6-dione

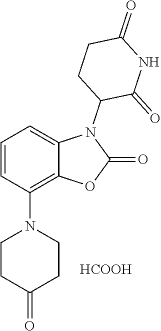

To a solution of 3-(2-oxo-7-(1,4-dioxa-8-azaspiro[4.5]decan-8-yl)benzo[d]oxazol-3(2H)-yl)piperidine-2,6-dione (1 g, 2.58 mmol) in FA (30 mL) at 70° C. for 3 h. The reaction was concentrated under vacuum. The resulting solution was quenched with Et₂O (25 mL) stirred at rt for 2 h. The resulting mixture was filtered. The filtrate cake was concentrated under vacuum. The residue was purified by combi-flash PE:EA=100:0 to 70:30 to give the product (670.3 mg, 75.6%) as yellow green solid. LCMS (ESI) m/e [M+1]⁺=344.

### Step 4: (1S,3R)-3-(3-((2-((R)-1′-(3-((S)-2,6-dioxopiperidin-3-yl)-2-oxo-2,3-dihydrobenzo[d]oxazol-7-yl)-3,3-dimethyl-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropyl carbamate and (1S,3R)-3-(3-((2-((R)-1′-(3-((R)-2,6-dioxopiperidin-3-yl)-2-oxo-2,3-dihydrobenzo[d]oxazol-7-yl)-3,3-dimethyl-[1,4′-bipiperidin]-4-yl)-4-fluoro-1,1-dioxido-2,3-dihydrobenzo[d]isoth-iazol-5-yl)amino)-1H-pyrazol-5-yl)cyclopentyl isopropyl carbamate

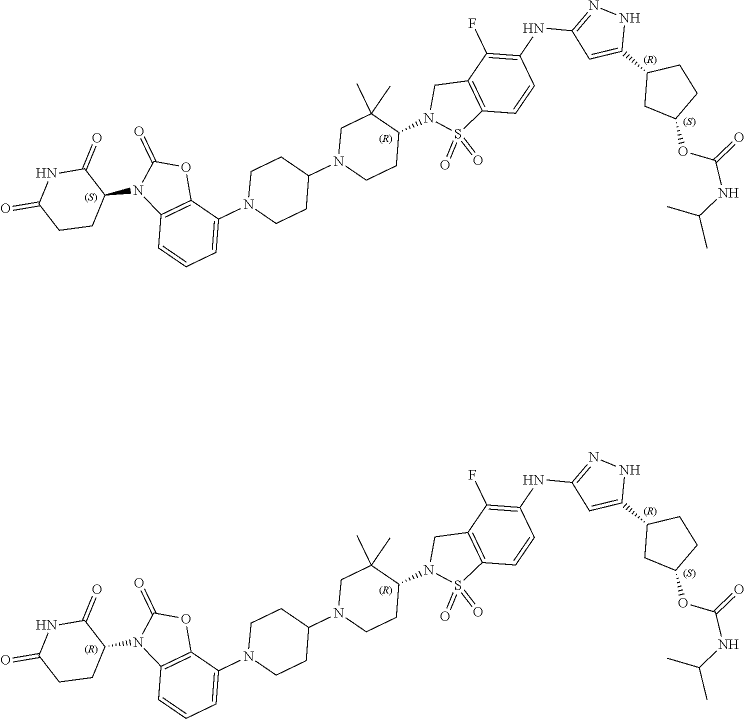

The titled compound was synthesized in the procedures similar to Example 63 in a racemate form, which was further separated by chiral HPLC to give:

Enantiomer 1 (Example 295, 100% ee); Retention time: 4.755 min. ¹H NMR (500 MHz, DMSO) δ 11.99 (s, 1H), 11.20 (s, 1H), 8.77 (s, 1H), 8.31 (s, 1H), 7.54 (d, J=8.6 Hz, 1H), 7.08 (t, J=8.1 Hz, 1H), 6.95 (d, J=7.3 Hz, 1H), 6.77 (d, J=7.9 Hz, 1H), 6.71 (d, J=8.3 Hz, 1H), 5.83 (s, 1H), 5.32 (q, J=12.9, 5.4 Hz, 1H), 5.00 (s, 1H), 4.57-4.49 (m, 2H), 3.72 (d, J=11.9 Hz, 2H), 3.63-3.51 (m, 1H), 3.21 (dd, J=12.1, 3.9 Hz, 1H), 3.12-2.99 (m, 2H), 2.93-2.57 (m, 8H), 2.28 (t, J=10.4 Hz, 1H), 2.18-1.98 (m, 4H), 1.93-1.53 (m, 9H), 1.10 (s, 3H), 1.03 (d, J=6.3 Hz, 6H), 0.94 (s, 3H). LC-MS (ESI): m/z [M+H]⁺=876.57.

Enantiomer 2 (Example 296, 100% ee); Retention time: 8.279 min. ¹H NMR (500 MHz, DMSO) δ 11.99 (s, 1H), 11.20 (s, 1H), 8.77 (s, 1H), 8.31 (s, 1H), 7.54 (d, J=8.6 Hz, 1H), 7.08 (t, J=8.1 Hz, 1H), 6.95 (d, J=7.3 Hz, 1H), 6.77 (d, J=7.9 Hz, 1H), 6.71 (d, J=8.3 Hz, 1H), 5.83 (s, 1H), 5.32 (q, J=12.9, 5.4 Hz, 1H), 5.00 (s, 1H), 4.57-4.49 (m, 2H), 3.72 (d, J=11.9 Hz, 2H), 3.63-3.51 (m, 1H), 3.21 (dd, J=12.1, 3.9 Hz, 1H), 3.12-2.99 (m, 2H), 2.93-2.57 (m, 8H), 2.28 (t, J=10.4 Hz, 1H), 2.18-1.98 (m, 4H), 1.93-1.53 (m, 9H), 1.10 (s, 3H), 1.03 (d, J=6.3 Hz, 6H), 0.94 (s, 3H). LC-MS (ESI): m/z [M+H]⁺=876.54.

Chiral analytical method: Column: YMC Amylose-SA 4.6*250 mm 5 μm Mobile phase: A for MtBE (0.1%2M NH3 MeOH) (%) and B for MeOH:DCM=50:50 (v/v); Gradient: Mobile Phase A: Mobile Phase B=40:60 (v/v); HPLC Equipment: Agilent 1260 Infinity II (ARD_LC-808); Column temperature: 25° C.

Chiral Prep-HPLC Condition: YMC Amylose-SA 4.6*250 mm 5 μm Mobile phase: A for MtBE (0.1%2M NH₃ MeOH)(%) and B for MeOH:DCM=50:50; Gradient: Mobile Phase A: Mobile Phase B=40:60 (v/v); Flow Rate: 1 mL/min, Wavelength: UV 250 nm and 280 nm, Prep-HPLC Equipment: Prep-HPLC Gilson (ARD_LC-808); Column temperature: 25° C.

### Example 297 and 298: (1S,3R)-3-(3-((2-((R)-1-((1-(3-((S)-2,6-dioxopiperidin-3-yl)-4-fluoro-2-oxo-2,3-dihydrobenzo[d]oxazol-6-yl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-((R)-1-((1-(3-((R)-2,6-dioxopiperidin-3-yl)-4-fluoro-2-oxo-2,3-dihydrobenzo[d]oxazol-6-yl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropylcarbamate

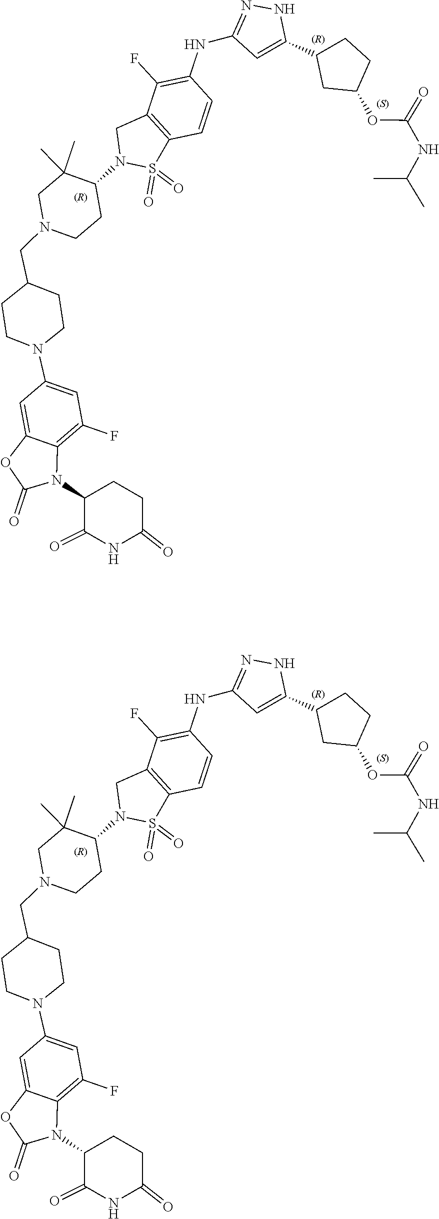

The racemic compound was synthesized in the procedures similar to Example 62, which was further separated by Chiral Prep-HPLC to afford the desired product:

Enantiomer 1 (Example 297, 100% ee); Retention time: 6.81 min. ¹H NMR (500 MHz, DMSO-d₆) δ 11.99 (s, 1H), 11.20 (s, 1H), 8.77 (s, 1H), 8.31 (t, J=5 Hz, 1H), 7.54 (d, J=10 Hz, 1H), 6.96-6.92 (m, 2H), 6.84-6.78 (m, 1H), 5.83 (s, 1H), 5.45-5.26 (m, 1H), 5.05-4.95 (m, 1H), 4.54 (s, 2H), 3.78-3.62 (m, 2H), 3.62-3.53 (m, 1H), 3.25-3.18 (m, 1H), 3.14-3.01 (m, 1H), 3.02-2.87 (m, 2H), 2.75-2.60 (m, 4H), 2.43-2.42 (m, 1H), 2.29-1.87 (m, 8H), 1.84-1.55 (m, 7H), 1.55-1.44 (m, 1H), 1.28-1.08 (m, 5H), 1.074-0.97 (m, 6H), 0.93 (s, 3H). LC-MS (ESI): m/z [M+H]⁺=908.

Enantiomer 1 (Example 298, 100% ee); Retention time: 8.76 min. ¹H NMR (500 MHz, DMSO-d₆) δ ¹H NMR (500 MHz, DMSO-d₆) δ 11.99 (s, 1H), 11.20 (s, 1H), 8.77 (s, 1H), 8.31 (t, J=5 Hz, 1H), 7.54 (d, J=10 Hz, 1H), 6.96-6.92 (m, 2H), 6.84-6.78 (m, 1H), 5.83 (s, 1H), 5.45-5.26 (m, 1H), 5.05-4.95 (m, 1H), 4.54 (s, 2H), 3.78-3.62 (m, 2H), 3.62-3.53 (m, 1H), 3.25-3.18 (m, 1H), 3.14-3.01 (m, 1H), 3.02-2.87 (m, 2H), 2.75-2.60 (m, 4H), 2.43-2.42 (m, 1H), 2.29-1.87 (m, 8H), 1.84-1.55 (m, 7H), 1.55-1.44 (m, 1H), 1.28-1.08 (m, 5H), 1.074-0.97 (m, 6H), 0.93 (s, 3H). LC-MS (ESI): m/z [M+H]⁺=908.

### Example 299: (1S,3R)-3-(3-((2-((4S)-1-((1-(3-(2,6-dioxopiperidin-3-yl)-2-oxo-2,3-dihydrobenzo[d]oxazol-6-yl)-4-methylpiperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropyl carbamate

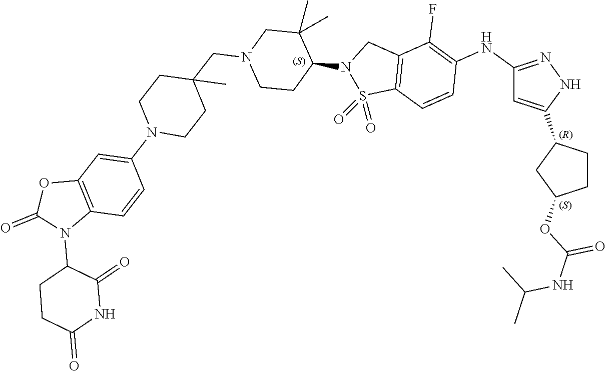

### Step 1: 1-(3-(2,6-dioxopiperidin-3-yl)-2-oxo-2,3-dihydrobenzo[d]oxazol-6-yl)-4-methylpiperidine-4-carbaldehyde

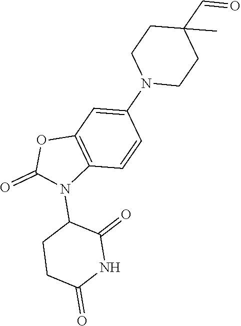 [SMILES: CC1(C=O)CCN(c2ccc3c(c2)oc(=O)n3C2CCC(=O)NC2=O)CC1]

The titled compound was synthesized in the procedures similar to Example 62. LC-MS (ESI): m/z [M+H]⁺=372.4

### Step 2: (1S,3R)-3-(3-((2-((4S)-1-((1-(3-(2,6-dioxopiperidin-3-yl)-2-oxo-2,3-dihydrobenzo[d]oxazol-6-yl)-4-methylpiperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropyl carbamate

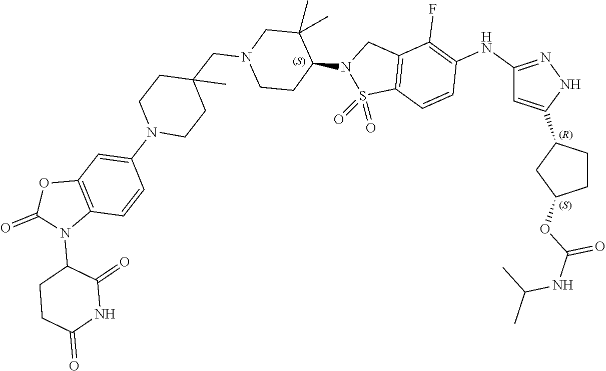

The titled compound was synthesized in the procedures similar to Example 62. ¹H NMR (500 MHz, DMSO) δ 11.98 (s, 1H), 11.17 (s, 1H), 8.76 (s, 1H), 8.30 (t, J=7.9 Hz, 1H), 7.53 (d, J=8.6 Hz, 1H), 7.09-7.03 (m, 2H), 6.94 (d, J=7.0 Hz, 1H), 6.79 (d, J=9.0 Hz, 1H), 5.83 (s, 1H), 5.29 (dd, J=12.9, 5.1 Hz, 1H), 5.00 (s, 1H), 4.54 (s, 2H), 3.63-3.53 (m, 1H), 3.18 (dd, J=12.1, 3.8 Hz, 1H), 3.11-3.02 (m, 1H), 2.99-2.83 (m, 4H), 2.72-2.60 (m, 5H), 2.38-2.28 (m, 2H), 2.25-2.09 (m, 5H), 2.06-1.98 (m, 1H), 1.96-1.85 (m, 1H), 1.77-1.55 (m, 5H), 1.46 (d, J=10.2 Hz, 1H), 1.39 (s, 2H), 1.31-1.02 (m, 3H), 1.03 (d, J=6.4 Hz, 6H), 0.96 (s, 3H), 0.91 (s, 3H). LC-MS (ESI): m/z [M+H]⁺=904.44.

### Example 300 and 301: (1S,3R)-3-(3-((2-((R)-1-((1-(3-((S)-2,6-dioxopiperidin-3-yl)-4-fluoro-2-oxo-2,3-dihydrobenzo[d]oxazol-6-yl)piperidin-4-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-((R)-1-((1-(3-((R)-2,6-dioxopiperidin-3-yl)-4-fluoro-2-oxo-2,3-dihydrobenzo[d]oxazol-6-yl)piperidin-4-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl isopropylcarbamate

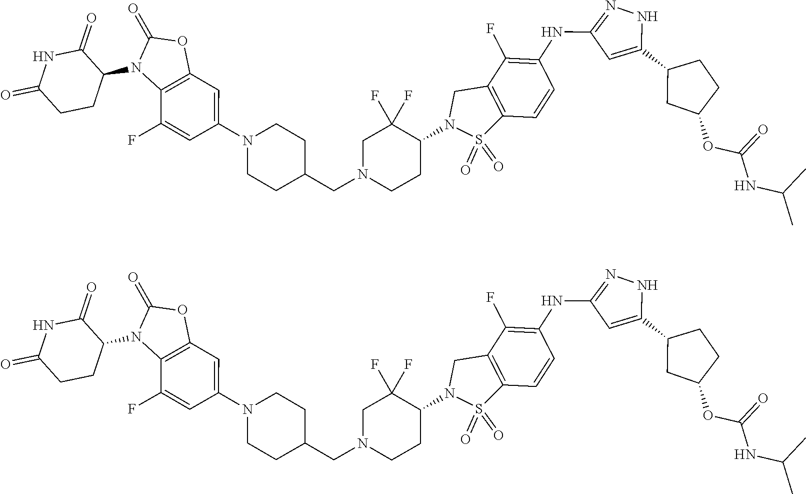 [SMILES: CC(C)NC(=O)O[C@H]1CC[C@@H](c2cc(Nc3ccc4c(c3F)CN([C@@H]3CCN(CC5CCN(c6cc(F)c7c(c6)oc(=O)n7[C@H]6CCC(=O)NC6=O)CC5)CC3(F)F)S4(=O)=O)n[nH]2)C1 ; CC(C)NC(=O)O[C@H]1CC[C@@H](c2cc(Nc3ccc4c(c3F)CN([C@@H]3CCN(CC5CCN(c6cc(F)c7c(c6)oc(=O)n7[C@@H]6CCC(=O)NC6=O)CC5)CC3(F)F)S4(=O)=O)n[nH]2)C1]

The titled compound was synthesized in the procedures similar to Example 279 in a racemate form, which was further separated by chiral HPLC to give:

Enantiomer 1 (Example 300, 100% ee); Retention time: 5.891 min. ¹H NMR (500 MHz, DMSO) δ 12.01 (s, 1H), 11.20 (s, 1H), 8.82 (s, 1H), 8.33 (t, J=8.0 Hz, 1H), 7.58 (d, J=8.7 Hz, 1H), 6.95 (d, J=1.6 Hz, 2H), 6.71 (d, J=13.9 Hz, 1H), 5.84 (s, 1H), 5.34 (s, 1H), 5.06-4.96 (m, 1H), 4.75 (d, J=14.4 Hz, 1H), 4.54 (d, J=12.9 Hz, 1H), 4.06-3.94 (m, 1H), 3.72-3.64 (m, 2H), 3.65-3.53-3.52 (m, 1H), 3.21-3.12 (m, 1H), 3.12-3.01 (m, 1H), 2.95-2.90 (m, 2H), 2.73-2.66 (m, 3H), 2.56-2.53 (m, 2H), 2.44-2.40 (m, 1H), 2.34-2.13 (m, 6H), 2.07-1.99 (m, 1H), 1.96-1.87 (m, 1H), 1.85-1.65 (m, 6H), 1.60-1.54 (m, 1H), 1.26-1.14 (s, 2H), 1.03 (d, J=6.3 Hz, 6H). LC-MS (ESI): m/z [M+H]⁺=916.4;

Enantiomer 2 (Example 301, 100% ee); Retention time: 7.813 min. ¹H NMR (500 MHz, DMSO) δ 12.01 (s, 1H), 11.20 (s, 1H), 8.82 (s, 1H), 8.33 (t, J=8.0 Hz, 1H), 7.58 (d, J=8.7 Hz, 1H), 6.95 (d, J=1.6 Hz, 2H), 6.71 (d, J=13.9 Hz, 1H), 5.84 (s, 1H), 5.34 (s, 1H), 5.06-4.96 (m, 1H), 4.75 (d, J=14.4 Hz, 1H), 4.54 (d, J=12.9 Hz, 1H), 4.06-3.94 (m, 1H), 3.72-3.64 (m, 2H), 3.65-3.53-3.52 (m, 1H), 3.21-3.12 (m, 1H), 3.12-3.01 (m, 1H), 2.95-2.90 (m, 2H), 2.73-2.66 (m, 3H), 2.56-2.53 (m, 2H), 2.44-2.40 (m, 1H), 2.34-2.13 (m, 6H), 2.07-1.99 (m, 1H), 1.96-1.87 (m, 1H), 1.85-1.65 (m, 6H), 1.60-1.54 (m, 1H), 1.26-1.14 (s, 2H), 1.03 (d, J=6.3 Hz, 6H). LC-MS (ESI): m/z [M+H]⁺=916.4;

Chiral analytical method: Column: ICHIRALPAK IC-5 4.6*150 mm 5 μm; Mobile phase: A for MtBE (0.1%2M NH3 MeOH) (%) and B for MeOH:DCM=50:50(%); Gradient: Mobile Phase A: Mobile Phase B=30:70 (v/v); HPLC Equipment: HPLC-Agilent, Back pressure: 100 bar; Column temperature: 35° C.

Chiral Prep-HPLC Condition: CHIRALPAK IC-5 250*20.0 mml, 5 μm; Mobile phase: A for MtBE (0.1%2M NH3 MeOH) (%) and B for MeOH:DCM=50:50; Gradient: Mobile Phase A: Mobile Phase B=30:70 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 302 and 303: (1S,3R)-3-(3-((2-((S)-1-((1-(3-((S)-2,6-dioxopiperidin-3-yl)-4-fluoro-2-oxo-2,3-dihydrobenzo[d]oxazol-6-yl)piperidin-4-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl isopropyl carbamate and (1S,3R)-3-(3-((2-((S)-1-((1-(3-((R)-2,6-dioxopiperidin-3-yl)-4-fluoro-2-oxo-2,3-dihydrobenzo[d]oxazol-6-yl)piperidin-4-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl isopropyl carbamate

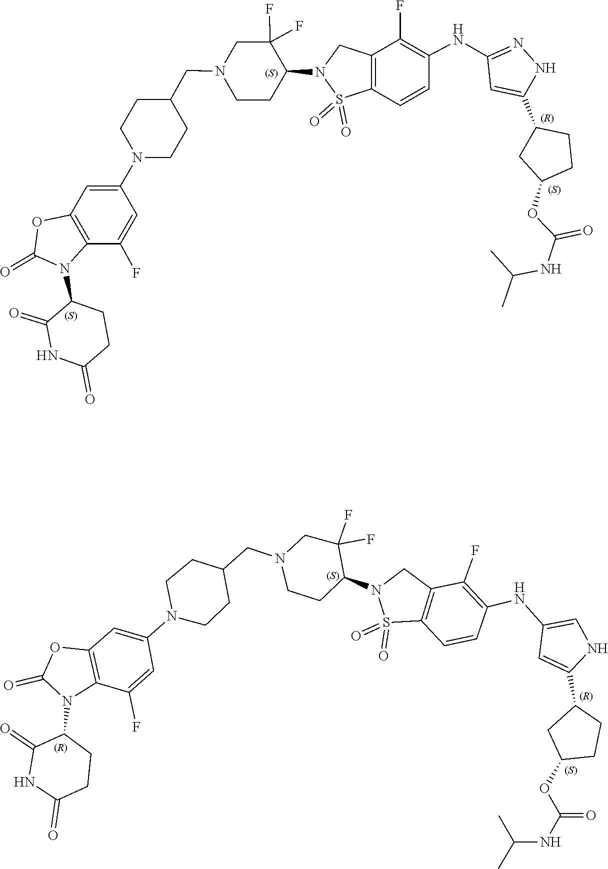

The titled compound was synthesized in the procedures similar to Example 279 in a racemate form, which was further separated by chiral HPLC to give:

Enantiomer 1 (Example 302, 100% ee); Retention time: 5.729 min. ¹H NMR (500 MHz, DMSO) δ 12.01 (s, 1H), 11.20 (s, 1H), 8.82 (s, 1H), 8.33 (t, J=8.1 Hz, 1H), 7.58 (d, J=8.7 Hz, 1H), 6.95 (d, J=1.5 Hz, 2H), 6.71 (d, J=14.5 Hz, 1H), 5.84 (s, 1H), 5.35 (s, 1H), 5.00 (s, 1H), 4.75 (d, J=14.4 Hz, 1H), 4.54 (d, J=13.1 Hz, 1H), 4.06-3.94 (m, 1H), 3.68 (d, J=9.3 Hz, 2H), 3.62-3.53 (m, 1H), 3.16 (t, J=9.6 Hz, 1H), 3.10-3.04 (m, 1H), 3.01-2.90 (m, 2H), 2.77-2.60 (m, 5H), 2.39-2.12 (m, 7H), 2.09-1.55 (m, 10H), 1.24-1.15 (m, 2H), 1.03 (d, J=6.3 Hz, 6H). LC-MS (ESI): m/z [M+H]⁺=915.92.

Enantiomer 2 (Example 303, 99.32% ee); Retention time: 7.596 min. ¹H NMR (500 MHz, DMSO) δ 12.01 (s, 1H), 11.20 (s, 1H), 8.82 (s, 1H), 8.33 (t, J=8.1 Hz, 1H), 7.58 (d, J=8.7 Hz, 1H), 6.95 (d, J=1.5 Hz, 2H), 6.71 (d, J=14.5 Hz, 1H), 5.84 (s, 1H), 5.35 (s, 1H), 5.00 (s, 1H), 4.75 (d, J=14.4 Hz, 1H), 4.54 (d, J=13.1 Hz, 1H), 4.06-3.94 (m, 1H), 3.68 (d, J=9.3 Hz, 2H), 3.62-3.53 (m, 1H), 3.16 (t, J=9.6 Hz, 1H), 3.10-3.04 (m, 1H), 3.01-2.90 (m, 2H), 2.77-2.60 (m, 5H), 2.39-2.12 (m, 7H), 2.09-1.55 (m, 10H), 1.24-1.15 (m, 2H), 1.03 (d, J=6.3 Hz, 6H). LC-MS (ESI): m/z [M+H]⁺=916.58.

Chiral analytical method: Column: i-Cellulose-5 4.6*250 mm 5 μm Mobile phase: A for MtBE (0.1%2M NH3 MeOH) (%) and B for MeOH:DCM=50:50 (v/v); Gradient: Mobile Phase A: Mobile Phase B=30:70 (v/v); HPLC Equipment: Agilent 1260 Infinity II (ARD_LC-808); Column temperature: 25° C.

Chiral Prep-HPLC Condition: i-Cellulose-5 4.6*250 mm 5 μm Mobile phase: A for MtBE (0.1%2M NH3 MeOH)(%) and B for MeOH:DCM=50:50; Gradient: Mobile Phase A: Mobile Phase B=30:70 (v/v); Flow Rate: 1 mL/min, Wavelength: UV 250 nm and 280 nm, Prep-HPLC Equipment: Prep-HPLC Gilson (ARD_LC-808); Column temperature: 25° C.

### Example 304 and 305: (1S,3R)-3-(3-((2-((S)-1′-(3-((S)-2,6-dioxopiperidin-3-yl)-4-fluoro-2-oxo-2,3-dihydrobenzo[d]oxazol-6-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-((S)-1′-(3-((R)-2,6-dioxopiperidin-3-yl)-4-fluoro-2-oxo-2,3-dihydrobenzo[d]oxazol-6-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

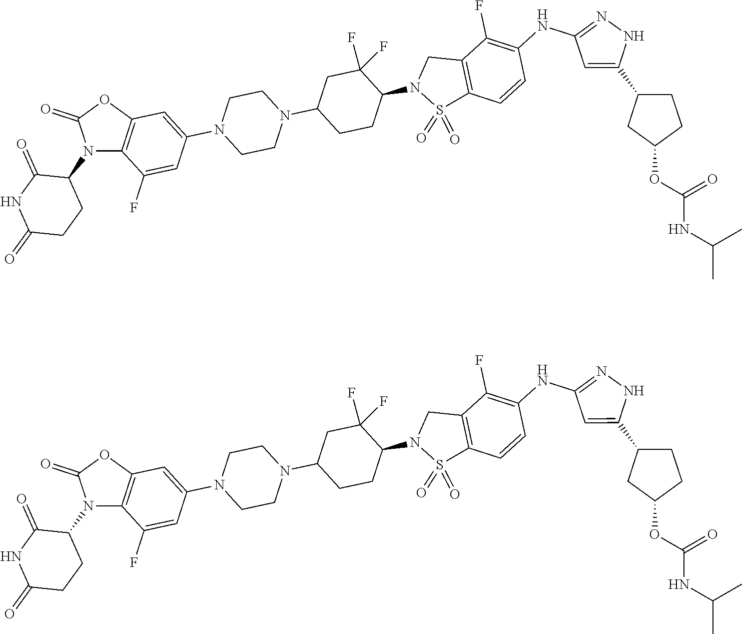 [SMILES: CC(C)NC(=O)O[C@H]1CC[C@@H](c2cc(Nc3ccc4c(c3F)CN([C@H]3CCC(N5CCN(c6cc(F)c7c(c6)oc(=O)n7[C@H]6CCC(=O)NC6=O)CC5)CC3(F)F)S4(=O)=O)n[nH]2)C1 ; CC(C)NC(=O)O[C@H]1CC[C@@H](c2cc(Nc3ccc4c(c3F)CN([C@H]3CCC(N5CCN(c6cc(F)c7c(c6)oc(=O)n7[C@@H]6CCC(=O)NC6=O)CC5)CC3(F)F)S4(=O)=O)n[nH]2)C1]

The titled compound was synthesized in the procedures similar to Example 279 in a racemate form, which was further separated to give:

Enantiomer 1 (Example 304, 100% ee); Retention time: 6.295 min. ¹H NMR (500 MHz, DMSO) δ 12.01 (s, 1H), 11.21 (s, 1H), 8.82 (s, 1H), 8.32 (t, J=8.1 Hz, 1H), 7.57 (d, J=8.7 Hz, 1H), 7.02-6.86 (m, 2H), 6.73 (d, J=14.1 Hz, 1H), 5.84 (s, 1H), 5.40 5.37 (m, 1H), 5.06-4.96 (m, 1H), 4.73 (d, J=14.5 Hz, 1H), 4.52 (d, J=13.6 Hz, 1H), 3.98 (s, 1H), 3.73 (d, J=11.0 Hz, 2H), 3.57 (s, 1H), 3.23-3.15 (m, 1H), 3.13-3.03 (m, 1H), 2.98-2.89 (m, 2H), 2.80-2.74 (m, 1H), 2.73-2.65 (m, 4H), 2.61-2.56 (m, 3H), 2.45-2.41 (m, 1H), 2.28-2.20 (m, 1H), 2.18-2.08 (m, 1H), 2.06-1.98 (m, 1H), 1.96-1.88 (m, 1H), 1.86-1.66 (m, 5H), 1.62 1.46 (m, 3H), 1.03 (d, J=6.2 Hz, 6H). LC-MS (ESI): m/z [M+H]⁺=902.4;

Enantiomer 1 (Example 305, 100% ee); Retention time: 7.737 min. ¹H NMR (500 MHz, DMSO) δ 12.01 (s, 1H), 11.21 (s, 1H), 8.82 (s, 1H), 8.32 (t, J=8.1 Hz, 1H), 7.57 (d, J=8.7 Hz, 1H), 7.02-6.86 (m, 2H), 6.73 (d, J=14.1 Hz, 1H), 5.84 (s, 1H), 5.40 5.37 (m, 1H), 5.06-4.96 (m, 1H), 4.73 (d, J=14.5 Hz, 1H), 4.52 (d, J=13.6 Hz, 1H), 3.98 (s, 1H), 3.73 (d, J=11.0 Hz, 2H), 3.57 (s, 1H), 3.23-3.15 (m, 1H), 3.13-3.03 (m, 1H), 2.98-2.89 (m, 2H), 2.80-2.74 (m, 1H), 2.73-2.65 (m, 4H), 2.61-2.56 (m, 3H), 2.45-2.41 (m, 1H), 2.28-2.20 (m, 1H), 2.18-2.08 (m, 1H), 2.06-1.98 (m, 1H), 1.96-1.88 (m, 1H), 1.86-1.66 (m, 5H), 1.62 1.46 (m, 3H), 1.03 (d, J=6.2 Hz, 6H). LC-MS (ESI): m/z [M+H]⁺=902.4;

Chiral analytical method: Column: ICHIRALPAK IC-5 4.6*150 mm 5 μm; Mobile phase: A for MtBE (0.1%2M NH3 MeOH) (%) and B for MeOH:DCM=50:50(%); Gradient: Mobile Phase A: Mobile Phase B=30:70 (v/v); HPLC Equipment: HPLC-Agilent, Back pressure: 100 bar; Column temperature: 35° C.

Chiral Prep-HPLC Condition: CHIRALPAK IC-5 250*20.0 mml, 5 μm; Mobile phase: A for MtBE (0.1%2M NH3 MeOH) (%) and B for MeOH:DCM=50:50; Gradient: Mobile Phase A: Mobile Phase B=30:70 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 306: (1S,3R)-3-(3-((2-((4S)-1-((1-(3-(2,6-dioxopiperidin-3-yl)-2-oxo-2,3-dihydrobenzo[d]oxazol-6-yl)azetidin-3-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl isopropyl carbamate

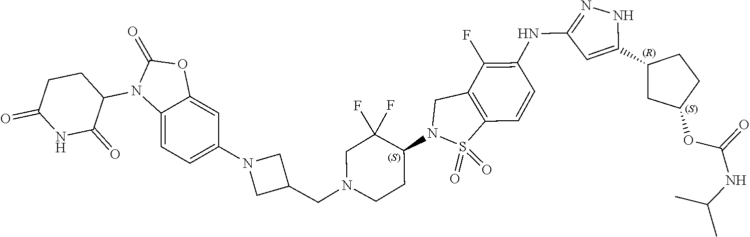

The titled compound was synthesized in the procedures similar to Example 101. ¹H NMR (500 MHz, DMSO) δ 11.99 (s, 1H), 11.14 (s, 1H), 8.78 (s, 1H), 8.33-8.31 (m, 1H), 7.54 (d, J=8.5 Hz, 1H), 7.11-7.01 (m, 1H), 6.99-6.89 (m, 1H), 6.62-6.52 (m, 1H), 6.31-6.20 (m, 1H), 5.85-5.83 (m, 1H), 5.36-5.21 (m, 5.3 Hz, 1H), 4.96 (s, 1H), 4.69 (d, J=14.5 Hz, 1H), 4.57-4.49 (m, 1H), 4.01-3.93 (m, 1H), 3.91-3.84 (m, 2H), 3.42-3.38 (m, 1H), 3.44-3.37 (m, 2H), 3.18-3.11 (m, 1H), 3.07-3.00 (m, 1H), 2.94-2.79 (m, 3H), 2.70-2.53 (m, 6H), 2.37-2.33 (m, 1H), 2.22-2.04 (m, 2H), 2.02-1.94 (m, 1H), 1.91-1.83 (m, 1H), 1.79-1.75 (m, 1H), 1.71-1.68 (m, 2H), 1.61-1.57 (m, 1H), 1.01 (d, J=5.8 Hz, 6H). LC-MS (ESI): m/z [M+H]⁺=870.29

### Example 307: (1S,3R)-3-(3-((2-((4R)-1′-(3-(2,6-dioxopiperidin-3-yl)-7-methoxy-2-oxo-2,3-dihydrobenzo[d]oxazol-6-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

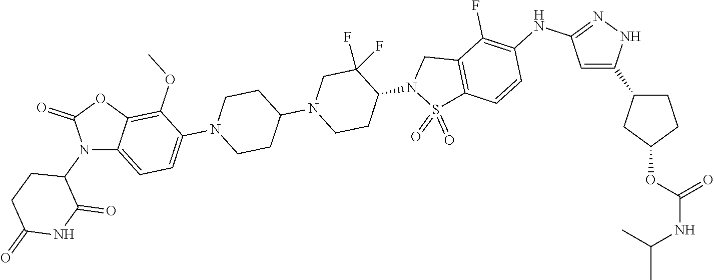 [SMILES: COc1c(N2CCC(N3CC[C@@H](N4Cc5c(ccc(Nc6cc([C@@H]7CC[C@H](OC(=O)NC(C)C)C7)[nH]n6)c5F)S4(=O)=O)C(F)(F)C3)CC2)ccc2c1oc(=O)n2C1CCC(=O)NC1=O]

The titled compound was synthesized in the procedures similar to Example 287. ¹H NMR (500 MHz, DMSO) δ 12.00 (s, 1H), 11.19 (s, 1H), 8.81 (s, 1H), 8.33 (s, 1H), 7.58 (d, J=8.6 Hz, 1H), 6.94 (d, J=8.0 Hz, 1H), 6.81 (dd, J=19.4, 8.5 Hz, 2H), 5.84 (s, 1H), 5.30 (dd, J=13.0, 5.2 Hz, 1H), 5.06-4.96 (m, 1H), 4.74 (d, J=14.4 Hz, 1H), 4.54 (d, J=13.1 Hz, 1H), 4.01 (s, 3H), 3.99-3.91 (m, 1H), 3.64-3.52 (m, 1H), 3.26-3.18 (m, 1H), 3.14-3.00 (m, 2H), 2.91-2.68 (m, 7H), 2.62-2.58 (s, 1H), 2.46-2.41 (m, 1H), 2.20-2.10 (m, 2H), 2.06-2.03 (m, 1H), 1.98-1.90 (m, 1H), 1.88-1.80 (m, 3H), 1.78-1.56 (m, 5H), 1.04 (d, J=5.8 Hz, 6H). LC-MS (ESI): m/z [M+H]⁺=914.4;

### Example 308: (1S,3R)-3-(3-((2-((4S)-1-((1-(3-(2,6-dioxopiperidin-3-yl)-2-oxo-2,3-dihydrobenzo[d]oxazol-6-yl)azetidin-3-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropyl carbamate

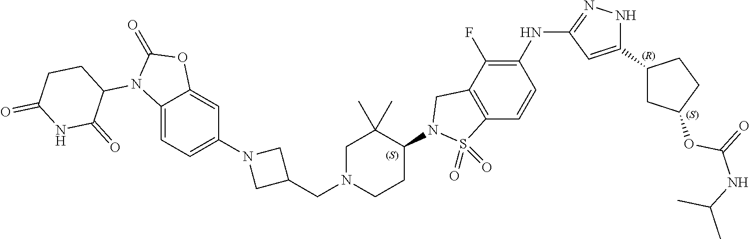

The titled compound was synthesized in the procedures similar to Example 101. ¹H NMR (500 MHz, DMSO) δ 11.99 (s, 1H), 11.16 (s, 1H), 8.76 (s, 1H), 8.29 (s, 1H), 7.54 (d, J=8.6 Hz, 1H), 7.04 (d, J=8.5 Hz, 1H), 6.94 (d, J=7.4 Hz, 1H), 6.53 (d, J=1.9 Hz, 1H), 6.24 (dd, J=8.5, 1.9 Hz, 1H), 5.84 (s, 1H), 5.27 (dd, J=13.0, 5.3 Hz, 1H), 5.00 (s, 1H), 4.59-4.49 (m, 2H), 3.90 (dd, J=12.5, 7.2 Hz, 2H), 3.58 (dd, J=12.9, 6.6 Hz, 1H), 3.43 (dd, J=12.9, 6.7 Hz, 2H), 3.22 (dd, J=12.1, 3.8 Hz, 1H), 3.12-3.02 (m, 1H), 2.98-2.83 (m, 3H), 2.70-2.61 (m, 3H), 2.59-2.53 (m, 3H), 2.19-2.00 (m, 4H), 1.96-1.86 (m, 2H), 1.79-1.56 (m, 3H), 1.50 (d, J=9.7 Hz, 1H), 1.11 (s, 3H), 1.06-1.00 (m, 6H), 0.93 (s, 3H). LC-MS (ESI): m/z [M+H]⁺=862.49.

### Example 309: (1S,3R)-3-(3-((2-((4S)-1-((1-(3-(2,6-dioxopiperidin-3-yl)-5-methoxy-2-oxo-2,3-dihydrobenzo[d]oxazol-6-yl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropylcarbamate

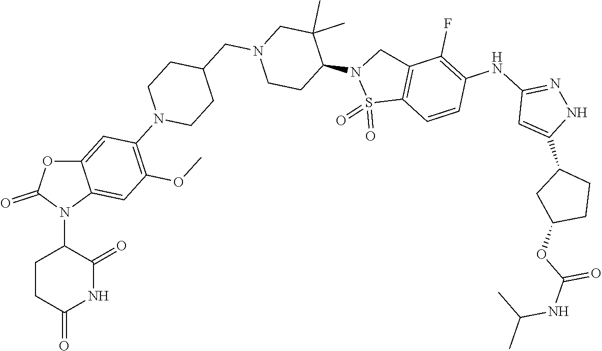 [SMILES: COc1cc2c(cc1N1CCC(CN3CC[C@H](N4Cc5c(ccc(Nc6cc([C@@H]7CC[C@H](OC(=O)NC(C)C)C7)[nH]n6)c5F)S4(=O)=O)C(C)(C)C3)CC1)oc(=O)n2C1CCC(=O)NC1=O]

### Step 1: 1-(3-(2,6-dioxopiperidin-3-yl)-5-methoxy-2-oxo-2,3-dihydrobenzo[d]oxazol-6-yl)piperidine-4-carbaldehyde

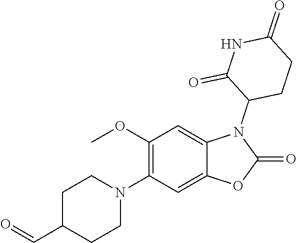 [SMILES: COc1cc2c(cc1N1CCC(C=O)CC1)oc(=O)n2C1CCC(=O)NC1=O]

The titled compound was synthesized in the procedures similar to Example 287. LC-MS (ESI): m/z [M+H]⁺=388.4.

### Step 2: (1S,3R)-3-(3-((2-((4S)-1-((1-(3-(2,6-dioxopiperidin-3-yl)-5-methoxy-2-oxo-2,3-dihydrobenzo[d]oxazol-6-yl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropylcarbamate

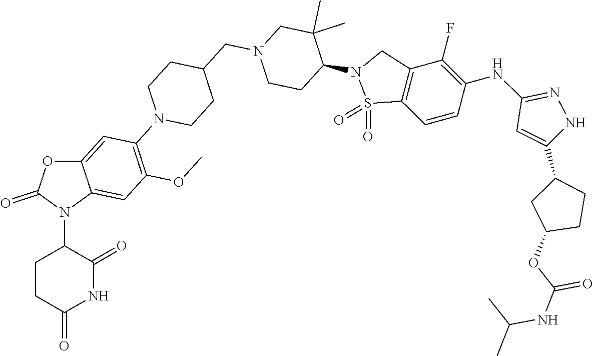 [SMILES: COc1cc2c(cc1N1CCC(CN3CC[C@H](N4Cc5c(ccc(Nc6cc([C@@H]7CC[C@H](OC(=O)NC(C)C)C7)[nH]n6)c5F)S4(=O)=O)C(C)(C)C3)CC1)oc(=O)n2C1CCC(=O)NC1=O]

The titled compound was synthesized in the procedures similar to Example 62. LC-MS (ESI): m/z [M+H]⁺=920.53; ¹H NMR (500 MHz, DMSO) δ=11.19 (s, 1H), 8.82 (s, 1H), 8.71 (s, 1H), 8.31 (t, J=7.8, 1H), 7.58 (d, J=8.6, 1H), 7.13 (s, 1H), 7.02 (s, 1H), 6.94 (d, J=7.3, 1H), 5.85 (s, 1H), 5.35 (dd, J=12.8, 5.2, 1H), 5.00 (s, 1H), 4.59 (s, 2H), 3.82 (s, 3H), 3.75 (s, 1H), 3.38-3.29 (m, 6H), 3.06 (m, 4H), 2.86 (m, 1H), 2.76 (m, 1H), 2.68 (m, 3H), 2.45 (m, 1H), 2.41-2.36 (m, 1H), 2.16-2.11 (m, 1H), 2.05-1.97 (m, 2H), 1.91 (m, 1H), 1.80 (m, 3H), 1.71 (m, 2H), 1.60 (m, 1H), 1.47 (m, 2H), 1.24 (s, 3H), 1.07 (s, 3H), 1.03 (m, 6H).

### Example 310 and 311: (1S,3R)-3-(3-((2-((S)-1-((1-(3-((R)-2,6-dioxopiperidin-3-yl)-4-fluoro-7-methyl-2-oxo-2,3-dihydrobenzo[d]oxazol-6-yl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-((S)-1-((1-(3-((S)-2,6-dioxopiperidin-3-yl)-4-fluoro-7-methyl-2-oxo-2,3-dihydrobenzo[d]oxazol-6-yl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropylcarbamate

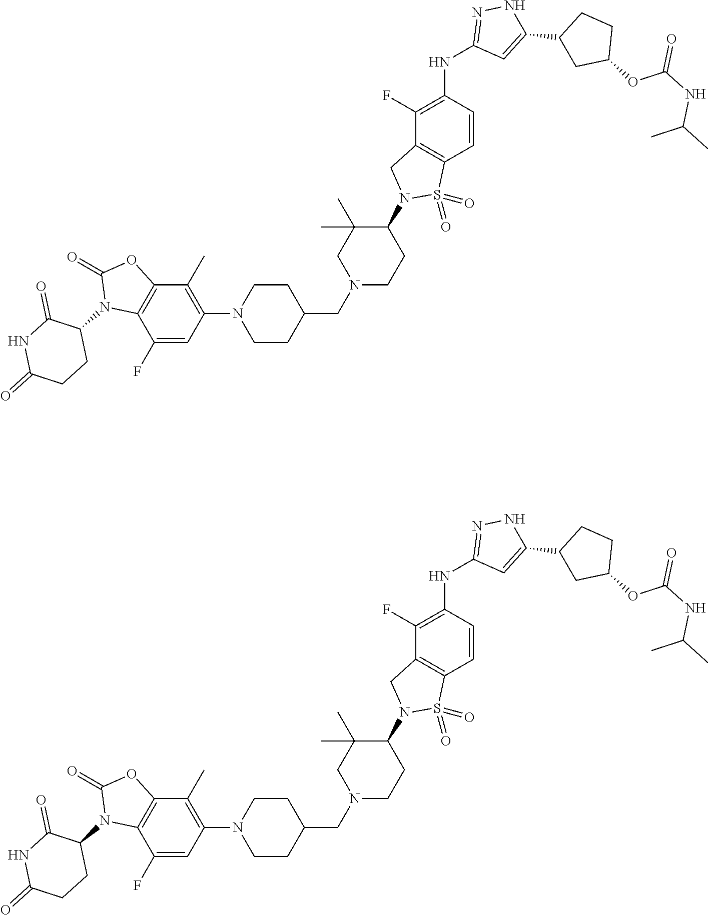

The racemic product was synthesized in the procedures similar to Example 280, which was further separated by Chiral Prep-HPLC to afford the desired product:

Enantiomer 1 (Example 311, 100% ee); Retention time: 7.093 min. 1H NMR (500 MHz, DMSO-d6) δ 11.98 (s, 1H), 11.21 (s, 1H), 8.76 (s, 1H), 8.31 (t, J=5 Hz, 1H), 7.54 (d, J=5 Hz, 1H), 6.97-6.91 (m, 1H), 6.83-6.80 (m, 1H), 5.83 (s, 1H), 5.45-5.30 (m, 1H), 5.05-4.95 (m, 1H), 4.54 (s, 2H), 3.63-3.52 (m, 1H), 3.25-3.19 (m, 1H), 3.17-3.00 (m, 6H), 2.99-2.90 (m, 2H), 2.70-2.55 (m, 3H), 2.47-2.43 (m, 1H), 2.29-2.09 (m, 8H), 2.06-1.95 (m, 2H), 1.94-1.88 (m, 1H), 1.87-1.66 (m, 5H), 1.66-1.55 (m, 2H), 1.54-1.47 (m, 1H), 1.36-1.22 (m, 2H), 1.12 (s, 3H), 1.07-0.98 (m, 6H), 0.94 (s, 3H). LC-MS (ESI): m/z [M+H]⁺=922.

Enantiomer 2 (Example 310, 100% ee) (Example 1b, 100% ee); Retention time: 9.244 min. 1H NMR (500 MHz, DMSO-d6) δ 11.98 (s, 1H), 11.21 (s, 1H), 8.76 (s, 1H), 8.31 (t, J=5 Hz, 1H), 7.54 (d, J=5 Hz, 1H), 6.97-6.91 (m, 1H), 6.83-6.80 (m, 1H), 5.83 (s, 1H), 5.45-5.30 (m, 1H), 5.05-4.95 (m, 1H), 4.54 (s, 2H), 3.63-3.52 (m, 1H), 3.25-3.19 (m, 1H), 3.17-3.00 (m, 6H), 2.99-2.90 (m, 2H), 2.70-2.55 (m, 3H), 2.47-2.43 (m, 1H), 2.29-2.09 (m, 8H), 2.06-1.95 (m, 2H), 1.94-1.88 (m, 1H), 1.87-1.66 (m, 5H), 1.66-1.55 (m, 2H), 1.54-1.47 (m, 1H), 1.36-1.22 (m, 2H), 1.12 (s, 3H), 1.07-0.98 (m, 6H), 0.94 (s, 3H). LC-MS (ESI): m/z [M+H]⁺=922.

Chiral analytical method: Column: Amylose-SA (4.6 mm×250 mm, 5 μm); Mobile phase: A for MtBE (0.2% NH3·MeOH) and B for DCM:MeOH=50:50; Gradient: Mobile Phase A: Mobile Phase B=30:70 (v/v); HPLC Equipment: HPLC-Agilent, Back pressure: 100 bar; Column temperature: 35° C.

Chiral Prep-HPLC Condition: Column: Amylose-SA (250*20.0 mm, 5 μm); Mobile phase: A for MtBE (0.2% NH3·MeOH) and B for DCM:MeOH=50:50; Gradient: Mobile Phase A: Mobile Phase B=30:70 (v/v); Flow Rate: 20 mL/min, Wave Length: UV 200 nm and 270 nm, Prep-HPLC Equipment: Prep-HPLC-Gilson, Back pressure: 100 bar; Column temperature: 25° C.

### Example 312 and 313: (1S,3R)-3-(3-((2-((S)-1-((1-(3-((S)-2,6-dioxopiperidin-3-yl)-4,7-difluoro-2-oxo-2,3-dihydrobenzo[d]oxazol-6-yl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-((S)-1-((1-(3-((R)-2,6-dioxopiperidin-3-yl)-4,7-difluoro-2-oxo-2,3-dihydrobenzo[d]oxazol-6-yl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropylcarbamate

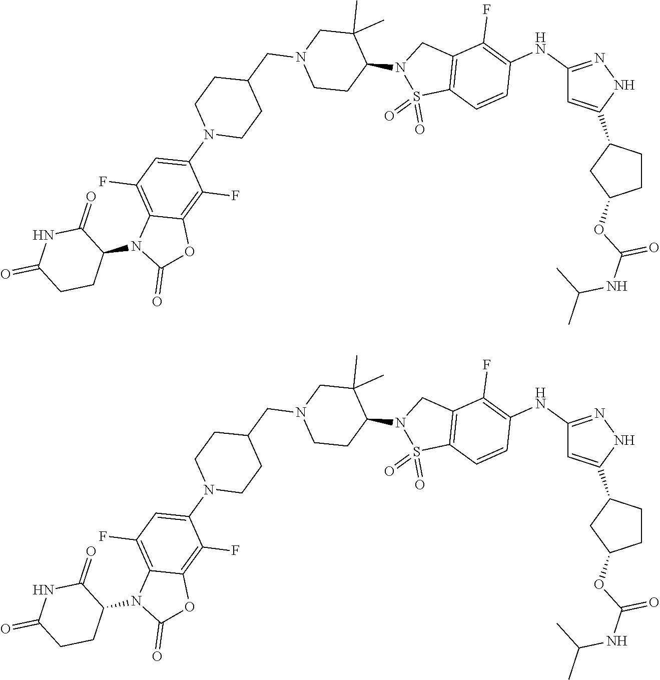 [SMILES: CC(C)NC(=O)O[C@H]1CC[C@@H](c2cc(Nc3ccc4c(c3F)CN([C@H]3CCN(CC5CCN(c6cc(F)c7c(oc(=O)n7[C@H]7CCC(=O)NC7=O)c6F)CC5)CC3(C)C)S4(=O)=O)n[nH]2)C1 ; CC(C)NC(=O)O[C@H]1CC[C@@H](c2cc(Nc3ccc4c(c3F)CN([C@H]3CCN(CC5CCN(c6cc(F)c7c(oc(=O)n7[C@@H]7CCC(=O)NC7=O)c6F)CC5)CC3(C)C)S4(=O)=O)n[nH]2)C1]

The titled compound was synthesized in the procedures similar to Example 282 in a racemate form, which was further separated by chiral HPLC to give:

Enantiomer 1 (Example 313, 100% ee); Retention time: 9.591 min. ¹H NMR (500 MHz, DMSO) δ 11.99 (s, 1H), 11.24 (s, 1H), 8.77 (s, 1H), 8.31 (t, J=7.8 Hz, 1H), 7.54 (d, J=8.6 Hz, 1H), 5.83 (s, 1H), 5.39 (s, 1H), 5.06-4.96 (m, 1H), 4.54 (s, 2H), 3.65-3.53-3.54 (m, 1H), 3.26-3.18 (m, 1H), 3.10-3.02 (m, 1H), 3.00-2.89 (m, 3H), 2.70-2.62 (m, 5H), 2.45-2.41 (m, 1H), 2.31-2.24 (m, 1H), 2.23-2.08 (m, 4H), 2.06-1.95 (m, 2H), 1.94-1.89 (m, 1H), 1.87-1.77 (m, 3H), 1.76-1.67 (m, 2H), 1.66-1.54 (m, 2H), 1.53-1.47 (m, 1H), 1.33-1.23 (m, 2H), 1.12 (s, 3H), 1.03 (d, J=5.8 Hz, 6H), 0.94 (s, 3H). LC-MS (ESI): m/z [M+H]⁺=926.4;

Enantiomer 2 (Example 312, 100% ee); Retention time: 7.626 min. ¹H NMR (500 MHz, DMSO) δ 11.99 (s, 1H), 11.24 (s, 1H), 8.77 (s, 1H), 8.31 (t, J=7.8 Hz, 1H), 7.54 (d, J=8.6 Hz, 1H), 5.83 (s, 1H), 5.39 (s, 1H), 5.06-4.96 (m, 1H), 4.54 (s, 2H), 3.65-3.53-3.54 (m, 1H), 3.26-3.18 (m, 1H), 3.10-3.02 (m, 1H), 3.00-2.89 (m, 3H), 2.70-2.62 (m, 5H), 2.45-2.41 (m, 1H), 2.31-2.24 (m, 1H), 2.23-2.08 (m, 4H), 2.06-1.95 (m, 2H), 1.94-1.89 (m, 1H), 1.87-1.77 (m, 3H), 1.76-1.67 (m, 2H), 1.66-1.54 (m, 2H), 1.53-1.47 (m, 1H), 1.33-1.23 (m, 2H), 1.12 (s, 3H), 1.03 (d, J=5.8 Hz, 6H), 0.94 (s, 3H). LC-MS (ESI): m/z [M+H]⁺=926.4;

Chiral analytical method: Column: ICHIRALPAK SA 4.6*150 mm 5 μm; Mobile phase: A for MtBE (0.1%2M NH3 MeOH) (%) and B for MeOH:DCM=50:50(%); Gradient: Mobile Phase A: Mobile Phase B=30:70 (v/v); HPLC Equipment: HPLC-Agilent, Back pressure: 100 bar; Column temperature: 35° C.

Chiral Prep-HPLC Condition: CHIRALPAK SA 250*20.0 mml, 5 μm; Mobile phase: A for MtBE (0.1%2M NH3 MeOH) (%) and B for MeOH:DCM=50:50; Gradient: Mobile Phase A: Mobile Phase B=30:70 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 314 and 315: (1S,3R)-3-(3-((2-((R)-1-((1-(3-((S)-2,6-dioxopiperidin-3-yl)-4,7-difluoro-2-oxo-2,3-dihydrobenzo[d]oxazol-6-yl)piperidin-4-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-((R)-1-((1-(3-((R)-2,6-dioxopiperidin-3-yl)-4,7-difluoro-2-oxo-2,3-dihydrobenzo[d]oxazol-6-yl)piperidin-4-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl isopropylcarbamate

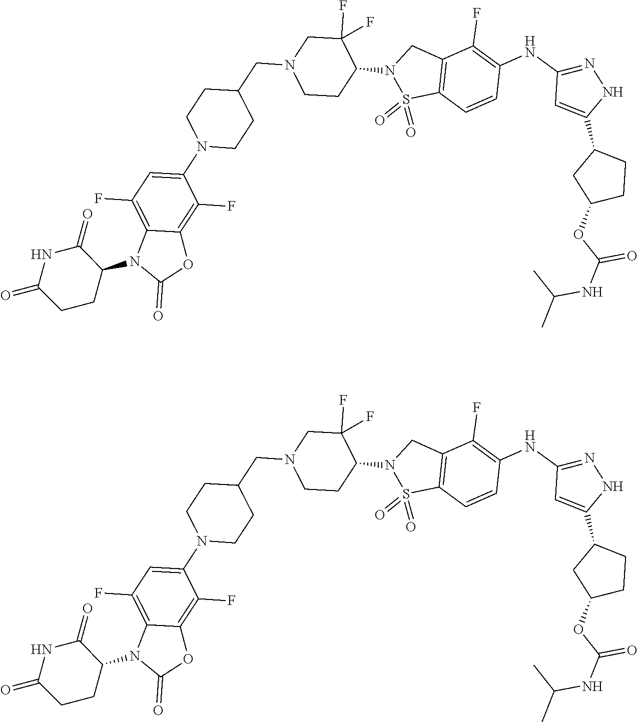 [SMILES: CC(C)NC(=O)O[C@H]1CC[C@@H](c2cc(Nc3ccc4c(c3F)CN([C@@H]3CCN(CC5CCN(c6cc(F)c7c(oc(=O)n7[C@H]7CCC(=O)NC7=O)c6F)CC5)CC3(F)F)S4(=O)=O)n[nH]2)C1 ; CC(C)NC(=O)O[C@H]1CC[C@@H](c2cc(Nc3ccc4c(c3F)CN([C@@H]3CCN(CC5CCN(c6cc(F)c7c(oc(=O)n7[C@@H]7CCC(=O)NC7=O)c6F)CC5)CC3(F)F)S4(=O)=O)n[nH]2)C1]

The titled compound was synthesized in the procedures similar to Example 282 in a racemate form, which was further separated by chiral HPLC to give:

Enantiomer 1 (Example 315, 100% ee); Retention time: 8.974 min. ¹H NMR (500 MHz, DMSO) δ 12.00 (s, 1H), 11.24 (s, 1H), 8.82 (s, 1H), 8.33 (t, J=8.1 Hz, 1H), 7.58 (d, J=8.7 Hz, 1H), 6.94 (s, 1H), 6.87 (dd, J=12.4, 6.2 Hz, 1H), 5.84 (s, 1H), 5.40 (s, 1H), 5.06-4.96 (m, 1H), 4.75 (d, J=14.5 Hz, 1H), 4.54 (d, J=12.9 Hz, 1H), 4.10-3.92 (m, 1H), 3.65-3.53-3.54 (m, 1H), 3.29-3.26 (m, 1H), 3.22-3.13 (m, 1H), 3.11-3.02 (m, 1H), 2.99-2.91 (m, 2H), 2.71-2.66 (m, 4H), 2.61-2.58 (s, 3H), 2.46-2.42 (m, 1H), 2.34-2.22 (m, 4H), 2.22-2.14 (m, 1H), 2.06-1.98 (m, 1H), 1.91 (s, 1H), 1.85-1.63 (m, 7H), 1.35-1.23 (m, 2H), 1.03 (d, J=6.2 Hz, 6H). LC-MS (ESI): m/z [M+H]⁺=934.4;

Enantiomer 1 (Example 314, 100% ee); Retention time: 8.198 min. ¹H NMR (500 MHz, DMSO) δ 12.00 (s, 1H), 11.24 (s, 1H), 8.82 (s, 1H), 8.33 (t, J=8.1 Hz, 1H), 7.58 (d, J=8.7 Hz, 1H), 6.94 (s, 1H), 6.87 (dd, J=12.4, 6.2 Hz, 1H), 5.84 (s, 1H), 5.40 (s, 1H), 5.06-4.96 (m, 1H), 4.75 (d, J=14.5 Hz, 1H), 4.54 (d, J=12.9 Hz, 1H), 4.10-3.92 (m, 1H), 3.65-3.53-3.54 (m, 1H), 3.29-3.26 (m, 1H), 3.22-3.13 (m, 1H), 3.11-3.02 (m, 1H), 2.99-2.91 (m, 2H), 2.71-2.66 (m, 4H), 2.61-2.58 (s, 3H), 2.46-2.42 (m, 1H), 2.34-2.22 (m, 4H), 2.22-2.14 (m, 1H), 2.06-1.98 (m, 1H), 1.91 (s, 1H), 1.85-1.63 (m, 7H), 1.35-1.23 (m, 2H), 1.03 (d, J=6.2 Hz, 6H). LC-MS (ESI): m/z [M+H]⁺=934.4;

Chiral analytical method: Column: ICHIRALPAK SA 4.6*150 mm 5 μm; Mobile phase: A for MtBE (0.1%2M NH3 MeOH) (%) and B for MeOH:DCM=50:50(%); Gradient: Mobile Phase A: Mobile Phase B=30:70 (v/v); HPLC Equipment: HPLC-Agilent, Back pressure: 100 bar; Column temperature: 35° C.

Chiral Prep-HPLC Condition: CHIRALPAK SA 250*20.0 mml, 5 μm; Mobile phase: A for MtBE (0.1%2M NH3 MeOH) (%) and B for MeOH:DCM=50:50; Gradient: Mobile Phase A: Mobile Phase B=30:70 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 316 and 317: (1S,3R)-3-(3-((2-((4R)-1′-(3-(2,6-dioxopiperidin-3-yl)-4,7-difluoro-2-oxo-2,3-dihydrobenzo[d]oxazol-6-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

### Step 1: (1S,3R)-3-(3-((2-((R)-1′-(3-((S)-2,6-dioxopiperidin-3-yl)-4,7-difluoro-2-oxo-2,3-dihydrobenzo[d]oxazol-6-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate and (1S,3R)-3-(3-((2-((R)-1′-(3-((R)-2,6-dioxopiperidin-3-yl)-4,7-difluoro-2-oxo-2,3-dihydrobenzo[d]oxazol-6-yl)-3,3-difluoro-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

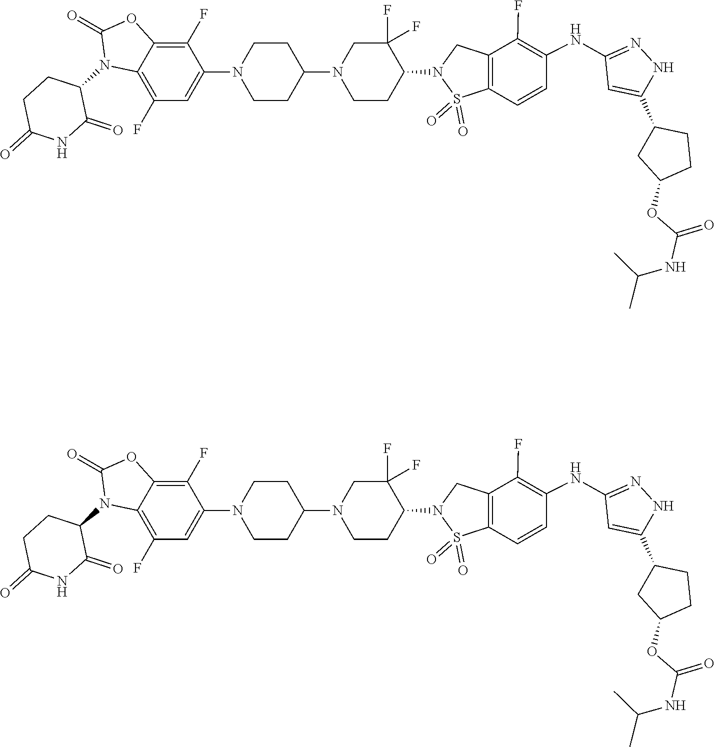 [SMILES: CC(C)NC(=O)O[C@H]1CC[C@@H](c2cc(Nc3ccc4c(c3F)CN([C@@H]3CCN(C5CCN(c6cc(F)c7c(oc(=O)n7[C@H]7CCC(=O)NC7=O)c6F)CC5)CC3(F)F)S4(=O)=O)n[nH]2)C1 ; CC(C)NC(=O)O[C@H]1CC[C@@H](c2cc(Nc3ccc4c(c3F)CN([C@@H]3CCN(C5CCN(c6cc(F)c7c(oc(=O)n7[C@@H]7CCC(=O)NC7=O)c6F)CC5)CC3(F)F)S4(=O)=O)n[nH]2)C1]

To a solution of 3-(4,7-difluoro-2-oxo-6-(4-oxopiperidin-1-yl)benzo[d]oxazol-3(2H)-yl)piperidine-2,6-dione (50.0 mg, 0.132 mmol) in DMA (3 mL), (1S,3R)-3-(3-((2-((R)-3,3-difluoropiperidin-4-yl)-4-fluoro-1,1-dioxido-2,3-dihydrobenzo[d]isothiazol-5-yl)amino)-1H-pyrazol-5-yl)cyclopentyl isopropylcarbamate (74 mg, 0.132 mmol) were added at rt. The resulting mixture was stirred for 10 mins. Then sodium cyanoborohydride (25.0 mg, 0.396 mmol) and acetic acid (one drop) were added into the mixture and stirred for 2 h. The reaction was detected by LCMS, after the residue was purified by silica gel chromatography, eluting with DCM/MeOH (10:1; R_{f}=0.35) to afford the crude product, then the crude product was purified by Prep-HPLC (Column: Sunfire C18, 19*150 mm, 5 μm; Mobile Phase A: H₂O (0.1% FA), Mobile Phase B: ACN; Flow Rate: 17 mL/min; Room Temperature; Detector: UV 214 & 254 nm; Gradient: 23% to 50% B in 11 min, 50% to 90% B in 0.2 min, 90% B hold 2 min, 90% to 25% B in 0.3 min, 23% B hold 1.5 min. to give the product (10.2 mg, 8.4% yield), which was further separated by Chiral Prep-HPLC to give:

Enantiomer 1 (Example 317, 99% ee); Retention time: 7.8 min. ¹H NMR (500 MHz, DMSO-d₆) δ 12.01 (s, 1H), 11.24 (s, 1H), 8.82 (s, 1H), 8.33 (t, J=8.0 Hz, 1H), 7.58 (d, J=8.7 Hz, 1H), 7.08-6.81 (m, 2H), 5.84 (s, 1H), 5.40 (s, 1H), 5.00 (s, 1H), 4.74 (d, J=14.4 Hz, 1H), 4.53 (d, J=13.2 Hz, 1H), 4.03-3.90 (m, 1H), 3.72-3.46 (m, 3H), 3.23 (d, J=9.6 Hz, 2H), 3.10-2.89 (m, 4H), 2.82-2.62 (m, 6H), 2.26 (d, J=5.1 Hz, 1H), 2.20-2.09 (m, 1H), 2.09-1.75 (m, 1H), 1.91 (m, 1H), 1.84 (m, 3H), 1.72 (m, 2H), 1.69-1.41 (m, 3H), 1.03 (d, J=6.1 Hz, 6H). LC-MS (ESI): m/z [M+H]⁺=920.52.

Enantiomer 2 (Example 316, 98.4% ee); Retention time: 11 min. ¹H NMR (500 MHz, DMSO-d₆) δ 12.01 (s, 1H), 11.24 (s, 1H), 8.82 (s, 1H), 8.33 (t, J=8.0 Hz, 1H), 7.58 (d, J=8.7 Hz, 1H), 7.08-6.81 (m, 2H), 5.84 (s, 1H), 5.40 (s, 1H), 4.97 (s, 1H), 4.74 (d, J=14.4 Hz, 1H), 4.53 (d, J=13.2 Hz, 1H), 4.03-3.90 (m, 1H), 3.72-3.46 (m, 3H), 3.22 (d, J=9.6 Hz, 2H), 3.10-2.89 (m, 4H), 2.82-2.62 (m, 6H), 2.26 (d, J=5.1 Hz, 1H), 2.20-2.09 (m, 1H), 2.09-1.75 (m, 1H), 1.91 (m, 1H), 1.84 (m, 3H), 1.74 (m, 2H), 1.69-1.41 (m, 3H), 1.03 (d, J=6.1 Hz, 6H). LC-MS (ESI): m/z [M+H]⁺=920.46.

Chiral analytical method: Column: Lux®5 μm i-Cellulose-5; Mobile phase: A for MtBE (0.1% NH3·MeOH) and B for DCM:MeOH=1:1; Gradient: Mobile Phase A: Mobile Phase B=50:50 (V/V); HPLC Equipment: Agilent 1260 Infinity II (ARD_LC-808); Column temperature: RT.

Chiral Prep-HPLC Condition: Column: Lux®5 μm i-Cellulose-5; Mobile phase: A for MtBE (0.2% NH3·MeOH) and B for DCM:MeOH=1:1; Gradient: Mobile Phase A: Mobile Phase B=50:50 (V/V); HPLC Equipment: Prep-HPLC Gilson (ARD_LC-815); Column temperature: RT.

### Example 318: (1S,3R)-3-(3-((2-((4S)-1-((1-(3-(2,6-dioxopiperidin-3-yl)-7-methoxy-2-oxo-2,3-dihydrobenzo[d]oxazol-6-yl)piperidin-4-yl)methyl)-3,3-dimethylpiperidin-4-yl)-4-fluoro-1,1-dioxido-2,3-dihydrobenzo[d]isothiazol-5-yl)amino)-1H-pyrazol-5-yl)cyclopentyl isopropylcarbamate

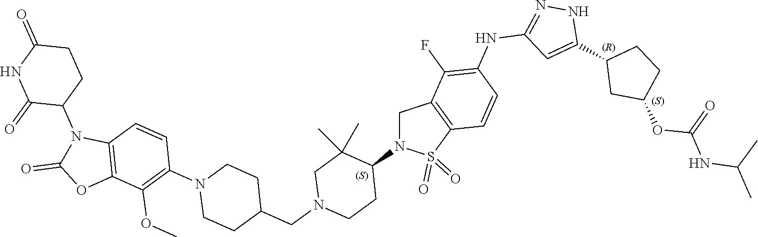

The titled compound was synthesized in the procedures similar to Example 286. ¹H NMR (500 MHz, DMSO) δ 11.99 (s, 1H), 11.19 (s, 1H), 8.76 (s, 1H), 8.31 (s, 1H), 7.54 (d, J=8.6 Hz, 1H), 6.94 (d, J=7.6 Hz, 1H), 6.81 (dd, J=19.1, 8.5 Hz, 2H), 5.83 (s, 1H), 5.30 (dd, J=13.1, 5.3 Hz, 1H), 5.00 (s, 1H), 4.54 (s, 2H), 4.00 (s, 3H), 3.58 (d, J=6.6 Hz, 1H), 3.30-3.19 (m, 3H), 3.13-3.03 (m, 1H), 3.00-2.84 (m, 2H), 2.70-2.57 (m, 6H), 2.21-1.72 (m, 12H), 1.60 (s, 2H), 1.51 (d, J=9.1 Hz, 1H), 1.34-1.24 (m, 2H), 1.13 (s, 3H), 1.07-0.98 (m, 6H), 0.94 (s, 3H). LC-MS (ESI): m/z [M+H]⁺=920.82.

### Example 319: (1S,3R)-3-(3-((2-((4R)-1-((1-(3-(2,6-dioxopiperidin-3-yl)-7-methoxy-2-oxo-2,3-dihydrobenzo[d]oxazol-6-yl)piperidin-4-yl)methyl)-3,3-difluoropiperidin-4-yl)-4-fluoro-1,1-dioxido-2,3-dihydrobenzo[d]isothiazol-5-yl)amino)-1H-pyrazol-5-yl)cyclopentyl isopropylcarbamate

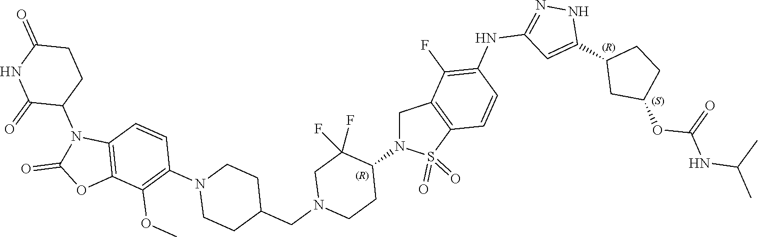

The titled compound was synthesized in the procedures similar to Example 286. ¹H NMR (500 MHz, DMSO) δ 12.01 (s, 1H), 11.19 (s, 1H), 8.82 (s, 1H), 8.33 (t, J=7.4 Hz, 1H), 7.58 (d, J=8.7 Hz, 1H), 6.94 (d, J=7.4 Hz, 1H), 6.81 (dd, J=19.1, 8.5 Hz, 2H), 5.84 (s, 1H), 5.30 (dd, J=13.0, 5.3 Hz, 1H), 5.00 (s, 1H), 4.75 (d, J=14.4 Hz, 1H), 4.54 (d, J=13.2 Hz, 1H), 4.07-3.93 (m, 4H), 3.63-3.51 (m, 1H), 3.30-3.25 (m, 2H), 3.17 (t, J=9.8 Hz, 1H), 3.11-3.01 (m, 1H), 2.98-2.82 (m, 2H), 2.72-2.56 (m, 6H), 2.38-2.29 (m, 3H), 2.25-2.09 (m, 2H), 2.0-5-1.98 (m, 9H), 1.35-1.24 (m, 2H), 1.07-0.98 (m, 6H). LC-MS (ESI): m/z [M+H]⁺=928.40.

### Example 320a and 320b: (1S,3R)-3-(3-(((S)-2-(1-((1-(4-((R)-2,6-dioxopiperidin-3-yl)-3,5-difluorophenyl)piperidin-4-yl)methyl)piperidin-4-yl)-4-fluoro-3-methyl-1,1-dioxido-2,3-dihydrobenzo[d]isothiazol-5-yl)amino)-1H-pyrazol-5-yl)cyclopentyl tert-butylcarbamate and (1S,3R)-3-(3-(((S)-2-(1-((1-(4-((S)-2,6-dioxopiperidin-3-yl)-3,5-difluorophenyl)piperidin-4-yl)methyl)piperidin-4-yl)-4-fluoro-3-methyl-1,1-dioxido-2,3-dihydrobenzo[d]isothiazol-5-yl)amino)-1H-pyrazol-5-yl)cyclopentyl tert-butylcarbamate

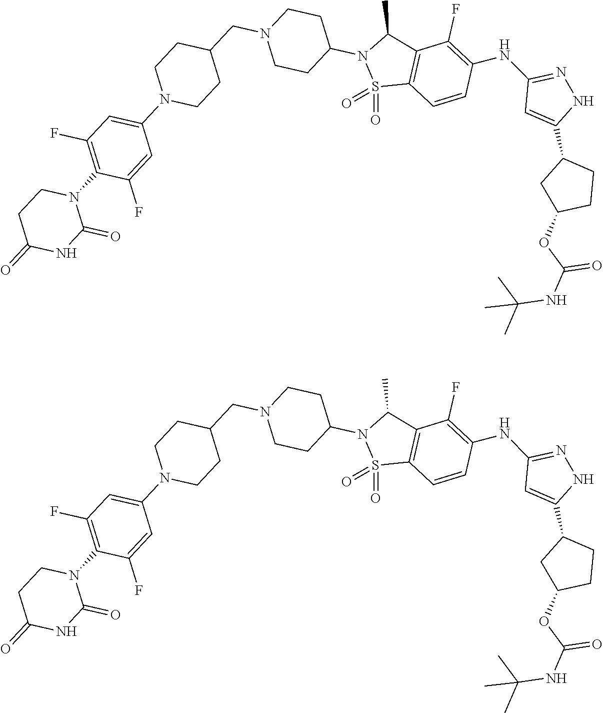

### Step 1: tert-butyl 4-(5-bromo-4-fluoro-3-methyl-1,1-dioxidobenzo[d]isothiazol-2(3H)-yl)piperidine-1-carboxylate

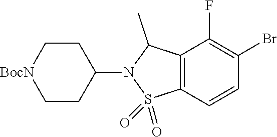 [SMILES: CC1c2c(ccc(Br)c2F)S(=O)(=O)N1C1CCN(C(=O)OC(C)(C)C)CC1]

To a stirring solution of 5-bromo-4-fluoro-3-methyl-2,3-dihydrobenzo[d]isothiazole 1,1-dioxide (5 g, 17.9 mmol) in toluene (100 mL) was added tert-butyl 4-hydroxypiperidine-1-carboxylate (5.7 g, 28.3 mmol) and 2-(tributylphosphoranylidene)acetonitrile (13.7 g, 56.6 mmol) at room temperature. The resulting solution was heated to 100° C. and stirred under nitrogen atmosphere for overnight. The solution was concentrated under reduced pressure, then triturated with PE/EA (4:1, 200 mL) and filtered to afford the product (3.2 g, 39% yield). LC-MS (ESI): m/z [M+H]⁺=463.2

### Step 2: tert-butyl 4-(5-((5-((1R,3S)-3-((tert-butylcarbamoyl)oxy)cyclopentyl)-1H-pyrazol-3-yl)amino)-4-fluoro-3-methyl-1,1-dioxidobenzo[d]isothiazol-2(3H)-yl)piperidine-1-carboxylate

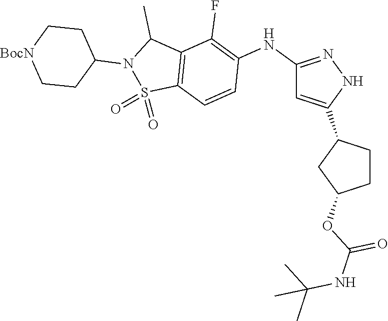 [SMILES: CC1c2c(ccc(Nc3cc([C@@H]4CC[C@H](OC(=O)NC(C)(C)C)C4)[nH]n3)c2F)S(=O)(=O)N1C1CCN(C(=O)OC(C)(C)C)CC1]

The titled compound was synthesized in the procedures similar to Example 1. LC-MS (ESI): m/z [M+H]⁺=649.5.

### Step 3: (1S,3R)-3-(3-((4-fluoro-3-methyl-1,1-dioxido-2-(piperidin-4-yl)-2,3-dihydrobenzo[d]isothiazol-5-yl)amino)-1H-pyrazol-5-yl)cyclopentyl tert-butylcarbamate

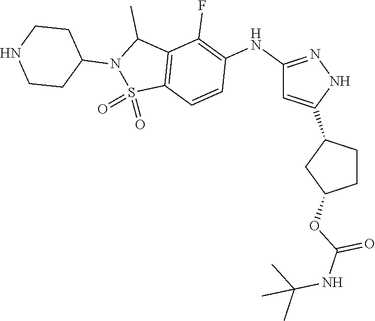 [SMILES: CC1c2c(ccc(Nc3cc([C@@H]4CC[C@H](OC(=O)NC(C)(C)C)C4)[nH]n3)c2F)S(=O)(=O)N1C1CCNCC1]

The titled compound was synthesized in the procedures similar to Example 1. LC-MS (ESI): m/z [M+H]⁺=549.5

### Step 4: (1S,3R)-3-(3-(((S)-2-(1-((1-(4-((R)-2,6-dioxopiperidin-3-yl)-3,5-difluorophenyl)piperidin-4-yl)methyl)piperidin-4-yl)-4-fluoro-3-methyl-1,1-dioxido-2,3-dihydrobenzo[d]isothiazol-5-yl)amino)-1H-pyrazol-5-yl)cyclopentyl tert-butylcarbamate and (1 S,3R)-3-(3-(((S)-2-(1-((1-(4-((S)-2,6-dioxopiperidin-3-yl)-3,5-difluorophenyl)piperidin-4-yl)methyl)piperidin-4-yl)-4-fluoro-3-methyl-1,1-dioxido-2,3-dihydrobenzo[d]isothiazol-5-yl)amino)-1H-pyrazol-5-yl)cyclopentyl tert-butylcarbamate

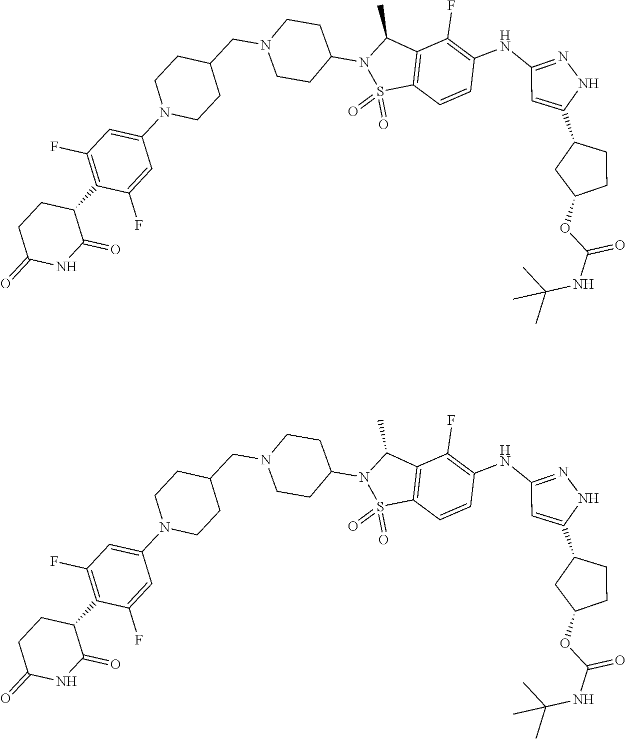 [SMILES: C[C@H]1c2c(ccc(Nc3cc([C@@H]4CC[C@H](OC(=O)NC(C)(C)C)C4)[nH]n3)c2F)S(=O)(=O)N1C1CCN(CC2CCN(c3cc(F)c([C@H]4CCC(=O)NC4=O)c(F)c3)CC2)CC1 ; C[C@@H]1c2c(ccc(Nc3cc([C@@H]4CC[C@H](OC(=O)NC(C)(C)C)C4)[nH]n3)c2F)S(=O)(=O)N1C1CCN(CC2CCN(c3cc(F)c([C@H]4CCC(=O)NC4=O)c(F)c3)CC2)CC1]

The titled compound was synthesized in the procedures similar to Example 1 in a racemate form, which was further separated by chiral HPLC to give:

Enantiomer 1 (Example 320a, 100% ee); Retention time: 4.99 min. ¹H NMR (500 MHz, DMSO) δ 12.01 (s, 1H), 10.87 (s, 1H), 9.04 (s, 1H), 8.79 (s, 1H), 8.28 (s, 1H), 7.51 (d, J=8.6 Hz, 1H), 6.75 (s, 1H), 6.66 (d, J=12.7 Hz, 2H), 5.84 (s, 1H), 4.98 (d, J=6.3 Hz, 2H), 4.05 (dd, J=12.3, 5.1 Hz, 1H), 3.82 (d, J=12.3 Hz, 3H), 3.65-3.53 (m, 2H), 3.15-2.95 (m, 5H), 2.85-2.75 (m, 3H), 2.54 (s, 4H), 2.47-2.42 (m, 1H), 2.42-2.34 (m, 1H), 2.33-2.24 (m, 1H), 2.23-1.99 (m, 5H), 1.99-1.85 (m, 2H), 1.85-1.77 (m, 2H), 1.75-1.65 (m, 2H), 1.61-1.53 (m, 3H), 1.30-1.22 (m, 2H), 1.21 (s, 9H). LC-MS (ESI): m/z [M+H]⁺=869.4

Enantiomer 2 (Example 320b, 100% ee); Retention time: 6.86 min. ¹H NMR (500 MHz, DMSO) δ 12.01 (s, 1H), 10.87 (s, 1H), 9.04 (s, 1H), 8.79 (s, 1H), 8.28 (s, 1H), 7.51 (d, J=8.6 Hz, 1H), 6.75 (s, 1H), 6.66 (d, J=12.7 Hz, 2H), 5.84 (s, 1H), 4.98 (d, J=6.3 Hz, 2H), 4.05 (dd, J=12.3, 5.1 Hz, 1H), 3.82 (d, J=12.3 Hz, 3H), 3.65-3.53 (m, 2H), 3.15-2.95 (m, 5H), 2.85-2.75 (m, 3H), 2.54 (s, 4H), 2.47-2.42 (m, 1H), 2.42-2.34 (m, 1H), 2.33-2.24 (m, 1H), 2.23-1.99 (m, 5H), 1.99-1.85 (m, 2H), 1.85-1.77 (m, 2H), 1.75-1.65 (m, 2H), 1.61-1.53 (m, 3H), 1.30-1.22 (m, 2H), 1.21 (s, 9H). LC-MS (ESI): m/z [M+H]⁺=869.4

Chiral analytical method: Column: ICHIRALPAK SA 4.6*150 mm 5 μm; Mobile phase: A for MtBE (0.1%2M NH3 MeOH) (%) and B for MeOH:DCM=50:50(%); Gradient: Mobile Phase A: Mobile Phase B=40:60 (v/v); HPLC Equipment: HPLC-Agilent, Back pressure: 100 bar; Column temperature: 35° C.

Chiral Prep-HPLC Condition: CHIRALPAK SA 250*20.0 mml, 5 μm; Mobile phase: A for MtBE (0.1%2M NH3 MeOH) (%) and B for MeOH:DCM=50:50; Gradient: Mobile Phase A: Mobile Phase B=40:60 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 321a and 321b: (1S,3R)-3-(3-(((S)-2-(1-((1-(4-(2,4-dioxotetrahydropyrimidin-1(2H)-yl)phenyl)piperidin-4-yl)methyl)piperidin-4-yl)-4-fluoro-3-methyl-1,1-dioxido-2,3-dihydrobenzo[d]isothiazol-5-yl)amino)-1H-pyrazol-5-yl)cyclopentyl tert-butylcarbamate and (1S,3R)-3-(3-(((R)-2-(1-((1-(4-(2,4-dioxotetrahydropyrimidin-1(2H)-yl)phenyl)piperidin-4-yl)methyl)piperidin-4-yl)-4-fluoro-3-methyl-1,1-dioxido-2,3-dihydrobenzo[d]isothiazol-5-yl)amino)-1H-pyrazol-5-yl)cyclopentyl tert-butylcarbamate

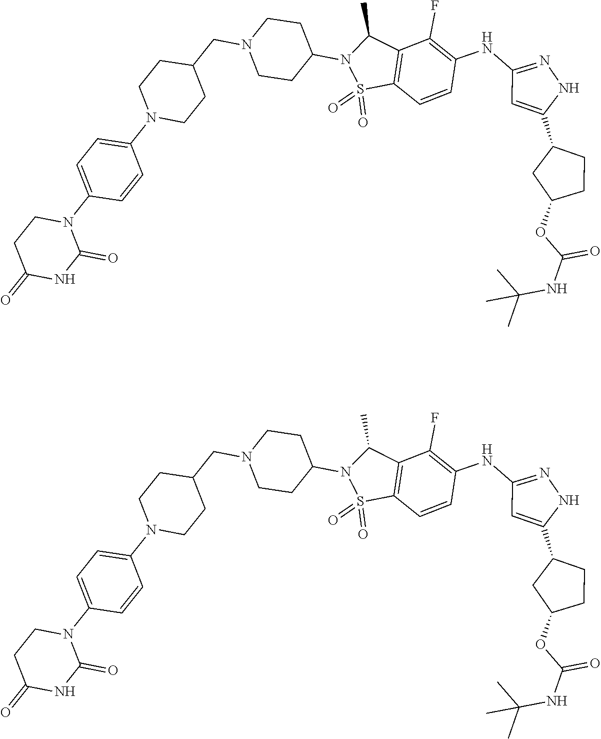 [SMILES: C[C@H]1c2c(ccc(Nc3cc([C@@H]4CC[C@H](OC(=O)NC(C)(C)C)C4)[nH]n3)c2F)S(=O)(=O)N1C1CCN(CC2CCN(c3ccc(N4CCC(=O)NC4=O)cc3)CC2)CC1 ; C[C@@H]1c2c(ccc(Nc3cc([C@@H]4CC[C@H](OC(=O)NC(C)(C)C)C4)[nH]n3)c2F)S(=O)(=O)N1C1CCN(CC2CCN(c3ccc(N4CCC(=O)NC4=O)cc3)CC2)CC1]

The titled compound was synthesized in the procedures similar to Example 70 in a racemate form, which was further separated by chiral HPLC to give:

Enantiomer 1 (Example 321a, 100% ee); Retention time: 6.5 min. ¹H NMR (500 MHz, DMSO) δ 11.98 (s, 1H), 10.25 (s, 1H), 8.74 (s, 1H), 8.26 (s, 1H), 7.47 (d, J=8.6 Hz, 1H), 7.13 (d, J=8.9 Hz, 2H), 6.92 (d, J=8.9 Hz, 2H), 6.76 (s, 1H), 5.83 (s, 1H), 5.05-4.95 (m, 2H), 3.72-3.64 (m, 4H), 3.44 (s, 1H), 3.12-3.01 (m, 1H), 2.92 (s, 2H), 2.70-2.62 (m, 5H), 2.45-2.44 (m, 1H), 2.20-2.14 (m, 2H), 2.11-1.83 (m, 7H), 1.79 (d, J=12.1 Hz, 2H), 1.75-1.63 (m, 3H), 1.62-1.56 (m, 1H), 1.53-1.44 (m, 3H), 1.21 (s, 9H). LC-MS (ESI): m/z [M+H]⁺=834.4

Enantiomer 2 (Example 321b, 100% ee); Retention time: 9.15 min. ¹H NMR (500 MHz, DMSO) δ 11.98 (s, 1H), 10.25 (s, 1H), 8.74 (s, 1H), 8.26 (s, 1H), 7.47 (d, J=8.6 Hz, 1H), 7.13 (d, J=8.9 Hz, 2H), 6.92 (d, J=8.9 Hz, 2H), 6.76 (s, 1H), 5.83 (s, 1H), 5.05-4.95 (m, 2H), 3.72-3.64 (m, 4H), 3.44 (s, 1H), 3.12-3.01 (m, 1H), 2.92 (s, 2H), 2.70-2.62 (m, 5H), 2.45-2.44 (m, 1H), 2.20-2.14 (m, 2H), 2.11-1.83 (m, 7H), 1.79 (d, J=12.1 Hz, 2H), 1.75-1.63 (m, 3H), 1.62-1.56 (m, 1H), 1.54-1.44 (m, 3H), 1.21 (s, 9H). LC-MS (ESI): m/z [M+H]⁺=834.4

Chiral analytical method: Column: ICHIRALPAK SA 4.6*150 mm 5 μm; Mobile phase: A for MtBE (0.1%2M NH3 MeOH) (%) and B for MeOH:DCM=50:50(%); Gradient: Mobile Phase A: Mobile Phase B=40:60 (v/v); HPLC Equipment: HPLC-Agilent, Back pressure: 100 bar; Column temperature: 35° C.

Chiral Prep-HPLC Condition: CHIRALPAK SA 250*20.0 mml, 5 μm; Mobile phase: A for MtBE (0.1%2M NH3 MeOH) (%) and B for MeOH:DCM=50:50; Gradient: Mobile Phase A: Mobile Phase B=40:60 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 322a and 322b: (1S,3R)-3-(3-(((3S)-2-(1-((1-(6-(2,6-dioxopiperidin-3-yl)-2-methylpyridin-3-yl)piperidin-4-yl)methyl)piperidin-4-yl)-4-fluoro-3-methyl-1,1-dioxido-2,3-dihydrobenzo[d]isothiazol-5-yl)amino)-1H-pyrazol-5-yl)cyclopentyl tert-butylcarbamate

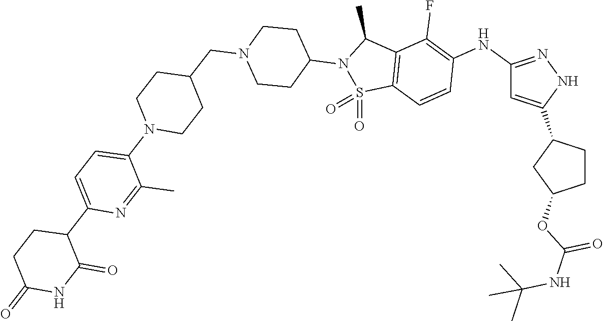 [SMILES: Cc1nc(C2CCC(=O)NC2=O)ccc1N1CCC(CN2CCC(N3[C@@H](C)c4c(ccc(Nc5cc([C@@H]6CC[C@H](OC(=O)NC(C)(C)C)C6)[nH]n5)c4F)S3(=O)=O)CC2)CC1]

The titled compound was synthesized in the procedures similar to Example 1 in a racemate form, which was further separated by chiral HPLC to give:

Enantiomer 1 (Example 322a, 100% ee); Retention time: 4.965 min. ¹H NMR (500 MHz, DMSO) δ 11.98 (s, 1H), 10.77 (s, 1H), 8.74 (s, 1H), 8.26 (s, 1H), 7.47 (d, J=8.6 Hz, 1H), 7.35 (d, J=8.1 Hz, 1H), 7.10 (d, J=8.1 Hz, 1H), 6.76 (s, 1H), 5.83 (s, 1H), 5.02-4.94 (m, 2H), 3.88 (dd, J=9.5, 5.3 Hz, 1H), 3.44 (s, 1H), 3.29 (s, 3H), 3.09-3.03 (m, 3H), 2.97-2.87 (m, 2H), 2.60-2.56 (m, 3H), 2.38 (s, 3H), 2.37-2.35 (m, 1H), 2.25-2.15 (m, 3H), 2.12-1.78 (m, 12H), 1.75-1.67 (m, 2H), 1.65-1.55 (m, 2H), 1.53-1.42 (m, 3H), 1.30-1.23 (m, 2H), 1.21 (s, 9H). LC-MS (ESI): m/z [M+H]⁺=848.4

Enantiomer 2 (Example 322b, 100% ee); Retention time: 7.873 min. ¹H NMR (500 MHz, DMSO) δ 11.98 (s, 1H), 10.77 (s, 1H), 8.74 (s, 1H), 8.26 (s, 1H), 7.47 (d, J=8.6 Hz, 1H), 7.35 (d, J=8.1 Hz, 1H), 7.10 (d, J=8.1 Hz, 1H), 6.76 (s, 1H), 5.83 (s, 1H), 5.02-4.94 (m, 2H), 3.88 (dd, J=9.5, 5.3 Hz, 1H), 3.44 (s, 1H), 3.29 (s, 3H), 3.09-3.03 (m, 3H), 2.97-2.87 (m, 2H), 2.60-2.56 (m, 3H), 2.38 (s, 3H), 2.37-2.35 (m, 1H), 2.25-2.15 (m, 3H), 2.12-1.78 (m, 12H), 1.75-1.67 (m, 2H), 1.65-1.55 (m, 2H), 1.53-1.42 (d, J=6.3 Hz, 3H), 1.30-1.23 (m, 2H), 1.21 (s, 9H). LC-MS (ESI): m/z [M+H]⁺=848.4

Chiral analytical method: Column: ICHIRALPAK SA 4.6*150 mm 5 μm; Mobile phase: A for MtBE (0.1%2M NH3 MeOH) (%) and B for MeOH:DCM=50:50(%); Gradient: Mobile Phase A: Mobile Phase B=40:60 (v/v); HPLC Equipment: HPLC-Agilent, Back pressure: 100 bar; Column temperature: 35° C.

Chiral Prep-HPLC Condition: CHIRALPAK SA 250*20.0 mml, 5 μm; Mobile phase: A for MtBE (0.1%2M NH3 MeOH) (%) and B for MeOH:DCM=50:50; Gradient: Mobile Phase A: Mobile Phase B=40:60 (v/v); Flow Rate: 18 mL/min, Wavelength: UV 250 nm and 280 nm, Prep-HPLC Equipment: Prep-HPLC Gilson (ARD_LC-815); Column temperature: 25° C.

### Example 323: (1R,3S)-3-(3-((2-(1-((1-(6-(2,6-dioxopiperidin-3-yl)-2-methylpyridin-3-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl tert-butylcarbamate

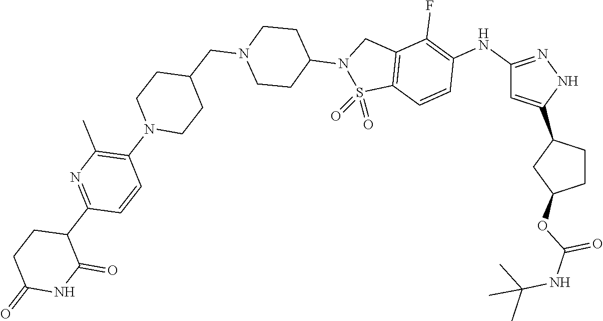 [SMILES: Cc1nc(C2CCC(=O)NC2=O)ccc1N1CCC(CN2CCC(N3Cc4c(ccc(Nc5cc([C@H]6CC[C@@H](OC(=O)NC(C)(C)C)C6)[nH]n5)c4F)S3(=O)=O)CC2)CC1]

The titled compound was synthesized in the procedures similar to Example 1. LC-MS (ESI): m/z [M+H]⁺=834.71; ¹H NMR (500 MHz, DMSO) δ=11.99 (s, 1H), 10.78 (s, 1H), 8.77 (s, 1H), 8.29 (s, 1H), 7.51 (d, J=8.6, 1H), 7.36 (d, J=8.2, 1H), 7.10 (d, J=8.1, 1H), 6.76 (s, 1H), 5.83 (s, 1H), 4.98 (s, 1H), 4.51 (s, 2H), 3.88 (dd, J=9.4, 5.3, 1H), 3.44 (m, 1H), 3.07 (m, 3H), 2.91 (d, J=11.0, 2H), 2.64-2.58 (m, 3H), 2.48-2.43 (m, 1H), 2.38 (m, 3H), 2.21 (m, 3H), 2.04 (m, 4H), 1.95-1.80 (m, 7H), 1.70 (m, 8.7, 2H), 1.62 (m, 2H), 1.31-1.23 (m, 3H), 1.22 (s, 9H).

### Example 324: (1S,3R)-3-(3-((2-(1-((1-(6-(2,6-dioxopiperidin-3-yl)-2-methylpyridin-3-yl)piperidin-4-yl)methyl)piperidin-4-yl)-4-fluoro-1,1-dioxido-2,3-dihydrobenzo[d]isothiazol-5-yl)amino)-1H-pyrazol-5-yl)cyclopentyl isopropylcarbamate

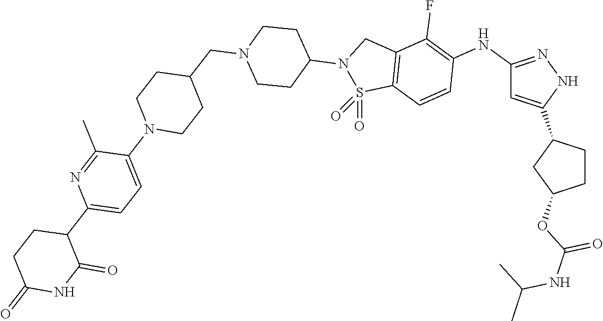 [SMILES: Cc1nc(C2CCC(=O)NC2=O)ccc1N1CCC(CN2CCC(N3Cc4c(ccc(Nc5cc([C@@H]6CC[C@H](OC(=O)NC(C)C)C6)[nH]n5)c4F)S3(=O)=O)CC2)CC1]

The titled compound was synthesized in the procedures similar to Example 323. ¹H NMR (500 MHz, DMSO) δ 11.99 (s, 1H), 10.77 (s, 1H), 8.76 (s, 1H), 8.30 (t, J=8.0 Hz, 1H), 7.51 (d, J=8.7 Hz, 1H), 7.36 (d, J=8.2 Hz, 1H), 7.10 (d, J=8.2 Hz, 1H), 6.94 (d, J=7.8 Hz, 1H), 5.83 (s, 1H), 5.06-4.96 (m, 1H), 4.51 (s, 2H), 3.92-3.86 (m, 1H), 3.63-3.55 (m, 1H), 3.48-3.38 (m, 1H), 3.22-2.97 (m, 3H), 2.91 (d, J=11.1 Hz, 2H), 2.64-2.54 (m, 4H), 2.47-2.44 (m, 1H), 2.38 (s, 3H), 2.26-2.16 (m, 3H), 2.13-1.98 (m, 4H), 1.95-1.77 (m, 7H), 1.77-1.55 (m, 4H), 1.32-1.22 (m, 2H), 1.03 (d, J=6.2 Hz, 6H). LC-MS (ESI): m/z [M+H]⁺=820.4;

### Example 325: (1S,3R)-3-(3-((2-(1′-(6-(2,6-dioxopiperidin-3-yl)-2-methylpyridin-3-yl)-[1,4′-bipiperidin]-4-yl)-4-fluoro-1,1-dioxido-2,3-dihydrobenzo[d]isothiazol-5-yl)amino)-1H-pyrazol-5-yl)cyclopentyl isopropylcarbamate

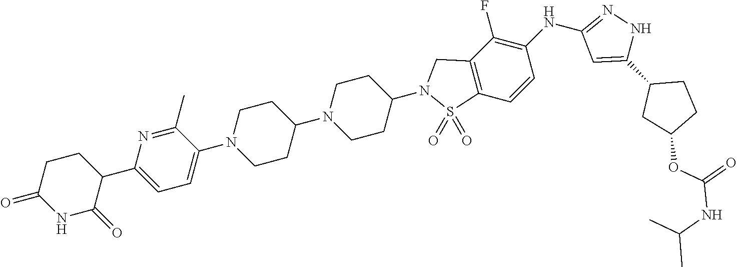 [SMILES: Cc1nc(C2CCC(=O)NC2=O)ccc1N1CCC(N2CCC(N3Cc4c(ccc(Nc5cc([C@@H]6CC[C@H](OC(=O)NC(C)C)C6)[nH]n5)c4F)S3(=O)=O)CC2)CC1]

The titled compound was synthesized in the procedures similar to Example 323. ¹H NMR (500 MHz, DMSO) δ 11.99 (s, 1H), 10.78 (s, 1H), 8.76 (s, 1H), 8.29 (s, 1H), 7.51 (d, J=8.6 Hz, 1H), 7.36 (d, J=8.3 Hz, 1H), 7.10 (d, J=8.1 Hz, 1H), 6.94 (d, J=7.0 Hz, 1H), 5.83 (s, 1H), 5.06-4.96 (m, 1H), 4.52 (s, 2H), 3.92-3.86 (m, 1H), 3.63-3.55 (m, 1H), 3.17-2.96 (m, 5H), 2.64-2.54 (m, 6H), 2.47-2.44 (m, 1H), 2.39 (s, 3H), 2.37-2.35 (m, 1H), 2.26-2.16 (m, 1H), 2.12-1.79 (m, 10H), 1.76-1.56 (m, 5H), 1.03 (d, J=6.1 Hz, 6H). LC-MS (ESI): m/z [M+H]⁺=806.4.

### Assays

### In-Cell Western (ICW) Protocol for CDK2 Degradation Measurement

The CDK2 half maximal degradation concentration (DC₅₀) values and degradation percentages of the compounds were measured with the following ICW assay procedure:

On Day 1, the culture medium for OVCAR8 cells was prepared using RPMI1640 with 10% FBS and 1% Pen Strep. The cells were cultivated in T-75 flasks at 37° C., 5% CO₂, 95% relative humidity, then washed by DPBS and digested by adding 0.25% Trypsin after reaching 80-90% confluence. The harvested OVCAR8 cells were seeded into 96-well plate (cell density: 40000 cells/well/90 uL medium, respectively) in medium and were incubated overnight at 37° C. and 5% CO₂.

On Day 2, the compounds were dissolved at 10 mM DMSO stock solution. 25 μL of stock solution was transferred to a 96-plate. 6-fold serial dilutions were performed via transferring 5 uL compound into 25 uL DMSO by using TECAN liquid handler. The plates were spined at room temperature at 1,000 RPM for 1 minute. 1 uL of diluted compound solution was transferred from compound source plate into 99 uL medium by 4081 MINI 96 Pipette. The plates were shaken at room temperature for 15 minutes. After 15 minutes, 10 uL diluted compound solution was transferred to 96-well cell culture plates using 4082 MINI 96 Pipette. Then the plates were incubated 24 hours at 37° C. and 5% CO₂.

On Day 3, after the treatment with compounds for 24 hours, the plates were removed from incubators, and then the medium in the plate was discarded. 100 uL 4% fixing buffer was added at room temperature for 20 min. Note: The 37% formaldehyde solution was diluted into 4% formaldehyde solution with TBS and stored in the dark.

The reaction was washed 3 times with 1×TBST buffer, then added 100 uL quench buffer (endogenous peroxidase blocking solution) at room temperature for 20 min.

The reaction was further washed 3 times with 1×TBST buffer, then added 200 uL 2.5% BSA blocking buffer at room temperature for 1 hour. Note: BSA was dissolved in 1×TBST.

The 2.5% BSA blocking buffer in the plate was discarded, and then 50 uL CDK2 antibody (CDK2 (78B2) Rabbit mAb (Vendor CST, Cat #2546S) 1:1000 dilution in 2.5% BSA blocking buffer) was added at 4° C. for overnight.

On Day 4, the reaction was washed 3 times with 1×TBST buffer, and then 50 ul anti-rabbit IgG HRP-linked Antibody ((Vendor CST, Cat #7074S) 1:1000 dilution in 2.5% BSA blocking buffer) was added at room temperature for 1.5 hours.

The reaction was washed 3 times with 1×TBST buffer, then 50 uL substrate was added at room temperature for 20-30 min.

50 ul quenching solution was added. The reaction was read on optical density (OD) at a wavelength of 450 nanometers (nm).

The degradation percentage of the compound was calculated by the following equation: degradation percentage of Compound, %=100-100×(Sample Signal-low control)/(High control-low control), wherein signal=each test compound group.

Half maximal degradation concentration (DC₅₀) values were calculated with a four-parameter logistic fit using GraphPad Prism.

Low control was OVCAR8 cell in plate, without compound & 1^{st} antibody, but with 2^{nd} antibody. High control was OVCAR8 cell in plate, without compound, but with 1^{st} antibody & 2^{nd} antibody, indicating microplate readings without CDK2 degradation.

Furthermore, the fix buffer was 4% (w/v) formaldehyde in 1×TBS. The quenching buffer was endogenous Peroxidase Blocking Buffer (Beyotime, #P0100A). The blocking buffer was 1×TBST containing 2.5% w/v BSA. The wash buffer was 1×TBST (containing 0.05% v/v Tween-20).

### OVCAR8/CDK2-HiBiT Protocol for CDK2 Degradation Measurement

The CDK2 half maximal degradation concentration (DC₅₀) values and degradation percentages of the compounds were measured with the following HiBiT assay procedure:

### OVCAR8 CDK2/HiBiT Cell Line Generation

To knock-in HiBiT sequence in the C-terminal of the endogenous CDK2 gene, OVCAR8 cells were co-transfected by CDK2-sgRNA-Cas9 plasmid and Pmax-CDK2-C-HiBiT plasmid, then followed by sorting and subcloning, the edited cells were seeded in 96 well plates at the concentration of 1 cell/cell, cultured for about 3 weeks-2 months to allow single clones formation. The formed clones were screened by Nano-Glo hibit-lgbit assay. OVCAR8 CDK2/HiBiT single clone was finally chosen to perform CDK2 degradation assay.

On Day 1, the culture medium for OVCAR8 CDK2/HiBiT cells was 90% RPMI1640+10% FBS+1% Penicillin-Streptomycin Liquid. The cells were cultivated in T-75 flasks at 37° C., 5% CO₂, 95% relative humidity, and then washed by DPBS and digested by adding 0.25% Trypsin after reaching 80-90% confluence.

The harvested OVCAR8/CDK2-HiBiT cells were seeded into 96-well plate (cell density: 5000 cells/well, respectively) in medium and were incubated overnight.

On Day 2, the compounds were dissolved at 10 mM DMSO stock solution. 25 μL of stock solution was transferred to a 96-well plate. 6-fold serial dilutions were performed via transferring 5 uL compound solution into 25 uL DMSO by using TECAN liquid handler.

The plates were spined at room temperature at 1,000 RPM for 1 minute. 1 uL of diluted compound solution was added into 99 uL medium by 4081 MINI 96 Pipette. The plates were shaken at room temperature for 15 minutes.

10 uL diluted compound solution was added to 96-well plates used 4082 MINI 96 Pipette. Then the plates were incubated 24 hours at 37° C. and 5% CO₂.

On Day 3, after 24-hour treatment with a compound, 10 ul Nano-Glo® HiBiT Lytic detection reagent (Vendor Promega, Cat #: N3040) was added at room temperature for 10 min in dark. Then the reaction was read on luminescence.

The Degradation percentage of the compound was calculated by the following equation: Degradation percentage of Compound, %=100-100×(Sample Signal-low control)/(High control-low control), wherein signal=each test compound group.

Half maximal degradation concentration (DC₅₀) values were calculated with a four-parameter logistic fit using GraphPad Prism.

Low control was OVCAR8/CDK2-HiBiT cell in plate, no compound, no Nano-Glo® HiBiT Lytic detection reagent.

High control was OVCAR8/CDK2-HiBiT cell in plate, no compound, but with Nano-Glo® HiBiT Lytic detection reagent, indicating microplate readings without CDK2 degradation.

### In-Cell Western (ICW) Protocol for CDK1 Degradation Measurement

The CDK1 half maximal degradation concentration (DC₅₀) values and degradation percentages of the compounds were measured with the following ICW assay procedure:

On Day 1, 90 μL of 1E4 OVCAR8 cells were seeded in culture medium in each well of a 96-well plate. The cells were incubated overnight at 37° C., 5% CO₂.

On Day 2, 1000× compound solution was prepared in DMSO: a compound in DMSO with 5-fold dilution by using 96-well storage plate from 10 mM to 0.1 M (Stock solution is 10 mM, starting final conc.=10 μM).

10× compound solution was prepared in growth medium: 1 μL serial diluted compound solution was transferred from last step to 99 μL growth medium, and mixed well.

10 μL of compound solution from last step was added to each well, spined at 1000 rpm for 1 min, then incubated at 37° C. for 24 h.

10 μL of growth medium with 1% DMSO was transferred and mixed well for high control and low control.

On Day 3, culture medium was removed and the cells were gently rinsed with 1×TBS.

The cells were fixed with 100 μL/well of Fix Buffer at RT for 20 min.

Note: During the incubation, the plates were sealed with parafilm. The media contained no more than 10% fetal serum otherwise efficiency of the cell crosslinking to the plate may be compromised.

The cultures were rinsed three times with 1×TBS.

100 μL/well of quenching buffer was added for 20 min to minimize endogenous peroxidase activity.

The cells were gently washed twice in 1×TBS for 5 minutes.

The cultures were incubated in 200 μL/well of Blocking Buffer at RT for 1 h.

The cells were labeled with 50 μL/well of primary antibody (Anti-CDK1 EPR165 antibody (Vendor abcam, Cat #ab133327) 1:1000 dilution) in Assay Diluent at 4° C. overnight.

On Day 4, the plate was rinsed three times in Wash Buffer. The cells were incubated with 50 μL/well of secondary antibody (Anti-rabbit IgG, HRP-linked Antibody (Vendor Cell signaling, Cat #7074S) 1:5000 dilution) in Assay Diluent at RT for 1 h.

The plate was rinsed three times in Wash Buffer.

The wash solution was aspirated, and then 50 μL HRP substrate was added to each well, and incubated at room temperature for 10 min.

50 μL of quenching solution was added to each well, and shaked gently for a few seconds.

The reaction was read regarding absorbance at 450 nm within 30 min.

The degradation percentage of the compound was calculated by the following equation: degradation percentage of Compound, %=100-100×(Sample Signal-low control)/(High control-low control), wherein signal=each test compound group.

Half maximal degradation concentration (DC₅₀) values were calculated with a four-parameter logistic fit using GraphPad Prism.

Low control is OVCAR8 cell in plate, without a compound & 1^{st} Ab, but with 2^{nd} Ab. High control is OVCAR8 cell in plate, without compound, but with 1^{st} Ab & 2^{nd} Ab, indicating microplate readings without CDK1 degradation.

The fix buffer was 4% (w/v) formaldehyde in 1×TBS. The quenching buffer was Endogenous Peroxidase Blocking Buffer (Beyotime, #P0100A). The blocking buffer was 1×TBS containing 2.5% w/v BSA. The wash buffer was 1×TBST (containing 0.05% v/v Tween-20). The assay diluent was 1×TBST containing 0.5% w/v BSA.

### Antiproliferation Assay

Antiproliferation was measured per the following assay procedure:

On Day 1, the culture medium for OVCAR3 cells was prepared using RPMI1640 with 20% FBS and human recombination insulin and 1% Pen Strep. The culture medium for TOV-21G cells was prepared with MCDB 105: Medium 199 (1:1) with 15% FBS and 1% Pen Strep.

The cells were cultivated in T-75 flasks at 37° C., 5% CO₂, 95% relative humidity, then washed by DPBS and digested by adding 0.25% Trypsin after reaching 80-90% confluence.

The harvested cells were seeded into 96-well plate (cell density: 1500 cells/well/90 uL medium for OVCAR3 and 200 cells/well/90 uL medium, respectively) in medium according to the plate map below and were incubated overnight at 37° C. and 5% CO₂.

On Day 2, compounds were dissolved at 10 mM DMSO stock solution. 25 μL of stock solution was transferred to a 96-plate. A 5-fold, 10-point dilution was performed via transferring 4 uL compound into 16 uL DMSO by using TECAN liquid handler. The plates were spined at room temperature at 1,000 RPM for 1 minute. 10 μL of diluted compound solution was transferred from compound source plate into the cell plate.

After 168-hour treatment with a compound, the plate was equilibrated at room temperature for 15 minutes. CellTiter-Glo® 2.0 reagent (Vendor Promega, Cat #G9242) was added into each well and the plates were spined at room temperature at 1,000 RPM for 5 minutes to be detected, and read by EnVision (Vendor Perkin Elmer, Cat #2105-0020).

The degradation percentage of the compound was calculated by the following equation: degradation percentage of Compound 100-100×(Signal-low control)/(High control-low control), wherein signal=each test compound group

Half maximal degradation concentration (DC₅₀) values were calculated with a four-parameter logistic fit using GraphPad Prism.

Low control=only culture buffer without cells; High control=Cell group with added DMSO and without compound, indicating microplate readings without antiproliferation effect.

### Biological Activity Tables

The CDK2 and CDK1 degradation data in OVCAR8 cells as measured for the Examples are shown in Table 1.

TABLE 1

| | CDK2 | | CDK2 | | CDK1 | |
|---|---|---|---|---|---|---|
| | degradation | CDK2 | degradation | CDK2 | degradation | CDK1 |
| | DC₅₀ | degradation | DC₅₀ | degradation | DC₅₀ | degradation |
| | (ICW) | Dmax | (HiBiT) | Dmax | (ICW) | Dmax |
| Example | (nM) | (ICW) | (nM) | (HiBiT) | (nM) | (ICW) |
| 1 | B | A | | | B | B |
| 2 | B | A | | | B | B |
| 3 | B | A | | | B | A |
| 4 | B | A | | | B | B |
| 5 | A | A | | | B | C |
| 6 | A | A | | | B | A |
| 7 | B | A | | | B | A |
| 8a | A | A | | | B | B |
| 8b | B | A | | | B | C |
| 9 | A | A | | | B | A |
| 10 | B | C | | | C | C |
| 11 | B | A | | | B | C |
| 12 | B | A | | | B | A |
| 13 | B | A | | | B | A |
| 14 | B | A | | | B | C |
| 15c | B | A | | | B | A |
| 15a | B | A | | | B | A |
| 15d | B | A | | | B | B |
| 15b | B | A | | | B | B |
| 16 | B | A | | | B | B |
| 17b | B | A | | | B | B |
| 17a | B | A | | | B | B |
| 18 | B | A | | | B | A |
| 19 | B | A | | | B | B |
| 20 | B | A | | | B | C |
| 21 | B | A | | | B | A |
| 22 | B | A | | | B | B |
| 23 | B | A | | | B | C |
| 24 | B | A | | | B | C |
| 25 | B | A | | | B | C |
| 26 | B | A | | | B | C |
| 27a | B | A | | | B | A |
| 27b | B | A | | | B | C |
| 28 | B | A | | | B | C |
| 29 | B | A | | | B | C |
| 30 | B | A | | | B | C |
| 31c | B | A | | | B | B |
| 31a | B | A | | | B | B |
| 31b | B | A | | | B | C |
| 31d | B | A | | | B | C |
| 32a | B | A | | | B | C |
| 32b | B | A | | | B | C |
| 33a | B | A | | | C | C |
| 33b | B | A | | | C | C |
| 34 | | | A | A | C | C |
| 35 | | | A | A | B | C |
| 36 | A | A | | | C | C |
| 37 | A | A | | | B | C |
| 38 | A | A | | | C | C |
| 39 | A | A | | | B | A |
| 40 | A | A | | | C | C |
| 41 | | | A | A | C | C |
| 42 | A | A | | | C | C |
| 43 | A | A | | | C | C |
| 44 | | | B | B | C | C |
| 45 | A | A | | | C | C |
| 46 | A | A | | | C | C |
| 47 | A | B | | | C | C |
| 48 | B | C | | | C | C |
| 49 | | | A | C | C | C |
| 50b | | | A | A | C | C |
| 50a | | | A | B | C | C |
| 51b | B | B | | | C | C |
| 51a | B | A | | | C | C |
| 52 | | | A | A | B | C |
| 53 | A | B | | | C | C |
| 54 | | | A | A | B | C |
| 55 | | | A | A | C | C |
| 56 | | | B | A | C | C |
| 57b | A | B | | | B | C |
| 57a | A | A | | | C | C |
| 58a | | | A | B | C | C |
| 58b | | | A | B | C | C |
| 59b | | | A | B | C | C |
| 59a | | | A | B | C | C |
| 60 | | | A | A | C | C |
| 61 | | | A | B | C | C |
| 62 | A | A | | | B | C |
| 63 | A | A | | | C | C |
| 64 | | | B | B | C | C |
| 65b | B | A | | | C | C |
| 65a | A | A | | | C | C |
| 66a | B | A | | | C | C |
| 66b | B | A | | | C | C |
| 67 | A | B | | | C | C |
| 68 | | | A | B | C | C |
| 69 | | | A | A | B | A |
| 70 | | | B | A | B | C |
| 71 | | | B | A | B | C |
| 72 | | | B | A | B | C |
| 73 | | | B | A | C | C |
| 74 | | | A | A | B | C |
| 75a | | | B | A | B | C |
| 75b | | | B | A | C | C |
| 76 | | | A | A | B | C |
| 77 | | | B | A | B | C |
| 78 | | | B | A | B | C |
| 79 | | | B | A | B | C |
| 80 | | | B | A | B | C |
| 81 | | | B | A | B | C |
| 82 | | | A | A | B | A |
| 83 | | | B | A | B | C |
| 84 | | | B | B | B | C |
| 85 | | | B | A | B | C |
| 86 | | | B | A | B | C |
| 87 | | | B | A | B | C |
| 88a | | | B | A | C | C |
| 88c | | | B | C | C | C |
| 88b | | | B | A | C | C |
| 89 | | | B | A | B | C |
| 90 | | | B | A | B | C |
| 91 | | | B | A | C | C |
| 92 | | | B | A | B | C |
| 93 | | | B | A | B | C |
| 94 | A | A | | | C | C |
| 95 | | | A | A | C | C |
| 96 | A | A | | | C | C |
| 97 | A | C | | | C | C |
| 98 | | | A | A | C | C |
| 99 | | | A | A | C | C |
| 100 | | | A | A | B | C |
| 101 | | | A | A | C | C |
| 102 | | | A | B | C | C |
| 103 | | | A | B | C | C |
| 104 | | | A | A | C | C |
| 105 | | | A | A | B | C |
| 106 | | | A | A | C | C |
| 107 | | | A | A | C | C |
| 108 | | | A | A | C | C |
| 109 | | | A | A | C | C |
| 110 | | | A | A | C | C |
| 111 | | | A | A | C | C |
| 112 | | | A | A | C | C |
| 113 | | | A | B | B | C |
| 114 | A | A | | | B | C |
| 115 | A | B | | | C | C |
| 116 | | | A | A | C | C |
| 117 | A | A | | | C | C |
| 118 | B | A | | | B | C |
| 119 | | | B | A | C | C |
| 120 | | | B | A | C | C |
| 121 | | | B | A | C | C |
| 122 | A | A | | | C | C |
| 123 | A | B | | | C | C |
| 124 | A | B | | | C | C |
| 125 | | | B | B | C | C |
| 126 | | | A | A | C | C |
| 127 | A | B | | | C | C |
| 128 | | | A | A | C | C |
| 129 | A | A | | | C | C |
| 130 | | | A | A | C | C |
| 131a | | | A | A | C | C |
| 131b | | | A | A | C | C |
| 131d | | | A | A | C | C |
| 131c | | | A | A | C | C |
| 132 | | | B | C | C | C |
| 133 | | | A | A | C | C |
| 134 | | | A | A | C | C |
| 135 | | | A | A | C | C |
| 136 | | | A | A | C | C |
| 137 | | | A | A | C | C |
| 138 | | | A | B | C | C |
| 139 | | | A | A | C | C |
| 140 | | | A | A | B | C |
| 141 | | | A | A | C | C |
| 142 | | | A | A | C | C |
| 143 | | | A | C | C | C |
| 144 | | | A | A | B | C |
| 145 | | | A | A | C | C |
| 146 | | | A | A | C | C |
| 147 | | | A | C | C | C |
| 148 | B | A | | | B | C |
| 149 | B | A | | | B | C |
| 150 | | | A | A | C | C |
| 151a | | | A | A | C | C |
| 151b | | | B | B | C | C |
| 152 | | | A | A | B | C |
| 153 | | | B | B | C | C |
| 154 | | | B | B | C | C |
| 155 | | | B | A | C | C |
| 156 | A | A | | | C | C |
| 157 | | | A | A | C | C |
| 158 | | | A | A | C | C |
| 159 | | | A | A | C | C |
| 160 | | | A | A | B | C |
| 161 | | | A | A | C | C |
| 162 | A | A | | | C | C |
| 163 | | | A | A | C | C |
| 164 | | | A | A | C | C |
| 165 | A | B | | | C | C |
| 166 | A | A | | | C | C |
| 167 | A | B | | | C | C |
| 168 | A | A | | | C | C |
| 169 | A | A | | | C | C |
| 170 | | | A | A | C | C |
| 171 | A | A | | | C | C |
| 172 | A | B | | | C | C |
| 173 | A | B | | | C | C |
| 174 | A | A | | | C | C |
| 175 | A | B | | | C | C |
| 176 | | | A | A | C | C |
| 177 | | | B | A | C | C |
| 178 | | | A | A | C | C |
| 179 | | | A | A | C | C |
| 180 | | | A | A | C | C |
| 181 | | | A | A | C | C |
| 182 | | | A | A | C | C |
| 183 | | | A | A | C | C |
| 184 | | | A | A | C | C |
| 185 | | | A | A | C | C |
| 186 | | | A | A | C | C |
| 187 | | | A | A | C | C |
| 188 | A | B | | | C | C |
| 189 | A | B | | | C | C |
| 190 | | | A | A | C | C |
| 191a | A | A | | | C | C |
| 191b | B | B | | | C | C |
| 192b | | | A | A | C | C |
| 192c | | | A | A | C | C |
| 192a | | | B | A | C | C |
| 193a | | | B | A | C | C |
| 193b | | | A | A | C | C |
| 194 | A | A | | | C | C |
| 195b | A | C | | | C | C |
| 195a | A | C | | | C | C |
| 196a | | | A | A | C | C |
| 196b | | | A | A | C | C |
| 197 | | | B | A | B | C |
| 198 | | | A | A | C | C |
| 199 | | | B | B | C | C |
| 200b | | | A | A | C | C |
| 200a | | | A | A | C | C |
| 201 | | | A | C | C | C |
| 202 | | | A | B | C | C |
| 203 | | | A | A | C | C |
| 204 | | | A | A | C | C |
| 205 | | | A | A | C | C |
| 206 | | | A | A | C | C |
| 207 | | | A | A | C | C |
| 208 | | | A | A | C | C |
| 209 | | | A | A | C | C |
| 210 | | | A | A | C | C |
| 211 | | | A | A | C | C |
| 212 | | | A | A | C | C |
| 213 | | | A | B | C | C |
| 214 | | | A | A | C | C |
| 215 | | | A | A | C | C |
| 216 | | | A | A | B | C |
| 217 | | | A | A | B | C |
| 218 | | | A | A | C | C |
| 219 | | | A | A | C | C |
| 220 | | | A | A | C | C |
| 221 | | | A | A | C | C |
| 222 | | | A | A | C | C |
| 223 | | | A | A | C | C |
| 224 | | | A | A | C | C |
| 225 | | | A | A | B | C |
| 226 | | | A | A | C | C |
| 227 | | | A | B | C | C |
| 228 | | | A | A | C | C |
| 229 | | | A | A | C | C |
| 230 | | | A | A | C | C |
| 231 | | | A | A | C | C |
| 232 | | | A | A | C | C |
| 233 | | | A | B | C | C |
| 234 | | | B | B | C | C |
| 235 | | | A | A | C | C |
| 236 | | | A | A | C | C |
| 237 | | | A | A | C | C |
| 238 | | | A | A | C | C |
| 239 | | | A | B | C | C |
| 240b | | | A | A | C | C |
| 240a | | | A | A | C | C |
| 241d | | | A | A | B | C |
| 241c | | | A | A | C | C |
| 241a | | | A | A | C | C |
| 241b | | | A | A | C | C |
| 242b | | | A | A | C | C |
| 242a | | | A | A | C | C |
| 243 | | | A | A | C | C |
| 244 | | | A | A | C | C |
| 245 | | | A | B | C | C |
| 246 | | | A | A | C | C |
| 247 | | | A | B | B | C |
| 248 | | | A | A | C | C |
| 249 | | | B | B | C | C |
| 250 | | | A | A | C | C |
| 251 | | | A | A | C | C |
| 252 | | | A | B | C | C |
| 253 | | | A | A | C | C |
| 254 | | | A | C | C | C |
| 255 | | | A | A | C | C |
| 256 | | | A | A | C | C |
| 257 | | | B | B | C | C |
| 258 | | | A | A | C | C |
| 259 | | | A | A | C | C |
| 260 | | | A | B | C | C |
| 261 | | | A | A | C | C |
| 262 | | | A | A | C | C |
| 263 | | | A | A | C | C |
| 264 | | | A | A | C | C |
| 265 | | | B | B | C | C |
| 266 | | | A | A | C | C |
| 267 | | | A | A | C | C |
| 268 | | | B | B | C | C |
| 269 | | | A | A | C | C |
| 270 | | | A | A | C | C |
| 271 | | | A | A | C | C |
| 272 | | | A | A | C | C |
| 273 | | | A | A | C | C |
| 274 | A | A | | | C | C |
| 275 | | | B | B | C | C |
| 276 | B | A | | | B | C |
| 277 | | | A | A | C | C |
| 278 | | | A | A | C | C |
| 279 | A | A | | | C | C |
| 279a | A | B | | | C | C |
| 279b | A | A | | | C | C |
| 280 | | | A | A | C | C |
| 281 | | | A | A | C | C |
| 282 | | | A | A | C | C |
| 283 | | | A | A | C | C |
| 284 | | | A | A | C | C |
| 285 | | | A | A | C | C |
| 286 | | | A | A | C | C |
| 287 | | | A | A | C | C |
| 288 | | | A | B | C | C |
| 289 | | | A | B | C | C |
| 290a | | | A | A | C | C |
| 290b | | | B | A | C | C |
| 291 | | | A | B | C | C |
| 292 | | | B | B | C | C |
| 293 | A | A | | | C | C |
| 294b | | | A | A | C | C |
| 294a | | | A | A | C | C |
| 295 | | | A | B | C | C |
| 296 | | | A | B | C | C |
| 297 | B | A | | | C | C |
| 298 | B | A | | | C | C |
| 299 | | | B | C | C | C |
| 300 | | | A | A | C | C |
| 301 | | | A | A | C | C |
| 302 | | | A | B | C | C |
| 303 | | | A | B | C | C |
| 304 | | | A | A | C | C |
| 305 | | | A | A | C | C |
| 306 | | | A | A | C | C |
| 307 | | | A | A | C | C |
| 308 | | | A | B | C | C |
| 309 | | | B | A | B | C |
| 310 | | | A | A | C | C |
| 311 | | | B | A | C | C |
| 312 | | | B | A | C | C |
| 313 | | | B | A | C | C |
| 314 | | | B | A | C | C |
| 315 | | | B | A | C | C |
| 316 | | | A | A | B | C |
| 317 | | | B | A | C | C |
| 318 | | | A | A | C | C |
| 319 | | | A | A | C | C |
| 320a | B | A | | | B | C |
| 320b | B | A | | | C | C |
| 321a | B | A | | | B | A |
| 321b | C | C | | | | |
| 322b | C | C | | | B | C |
| 322a | B | A | | | C | C |
| 323 | B | A | | | B | C |
| 324 | B | A | | | C | C |
| 325 | B | A | | | B | C |

The activity in Table 1 is categorized as “A”, “B”, and “C” based on the corresponding value according to the following rules.

| | CDK2 | CDK2 | CDK1 | CDK2 | CDK2 | CDK1 |
|---|---|---|---|---|---|---|
| | degradation | degradation | degradation | degradation | degradation | degradation |
| | DC₅₀ | DC₅₀ | DC₅₀ | Dmax | Dmax | Dmax |
| Categorized | (ICW) | (HiBiT) | (ICW) | (ICW) | (HiBiT) | (ICW) |
| A | No more | No more | No more | 90%-105% | 90%-105% | 90%-105% |
| | than 1 nM | than 1 nM | than 10 nM | | | |
| B | More than | More than | More than | 85%-89% | 85%-89% | 85%-89% |
| | 1 nM to no | 1 nM to no | 10 nM to | | | |
| | more than | more than | no more | | | |
| | 10 nM | 10 nM | than 10 uM | | | |
| C | More than | More than | More than | 0%-84% | 0%-84% | 0%-84% |
| | 10 nM to | 10 nM to | 10 uM to | | | |
| | 10 uM | 10 uM | 10000 uM | | | |

As demonstrated by the data in Table 1, the inventors surprisingly and unexpectedly discovered that the exemplary compounds in Table 1 modulate the activity of CDK2.

Unless otherwise specified, to the extent that there is a discrepancy between a depicted chemical structure of a compound provided herein and a chemical name of a compound provided herein, the chemical structure shall control.

A number of references have been cited, the disclosures of which are incorporated herein by reference in their entirety.

1-25. (canceled)

26. A compound of formula (I): 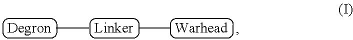
or a pharmaceutically acceptable salt thereof, wherein:
the Warhead is 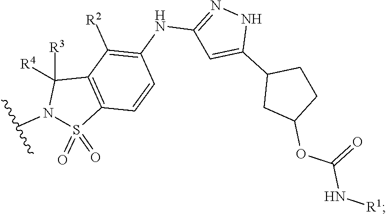
R¹ is isopropyl or tert-butyl; R² is F; and R³ and R⁴ are hydrogen;
the Linker is 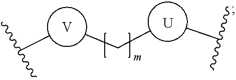
and
U and V are 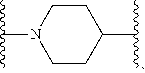 [SMILES: *C1CCN(*)CC1]
each of which is independently optionally substituted by one or two groups selected from F and methyl;
m is 1; and
the Degron is 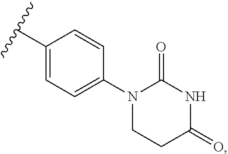 [SMILES: *c1ccc(N2CCC(=O)NC2=O)cc1]
optionally substituted by one or two groups selected from F and methyl.

27. The compound of claim 26, wherein R¹ is tert-butyl.

28. The compound of claim 27, wherein the compound has the structure of 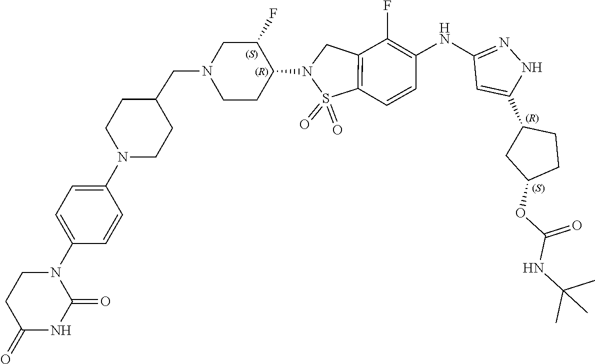
or a pharmaceutically acceptable salt thereof.

29. The compound of claim 27, wherein the compound has the structure of 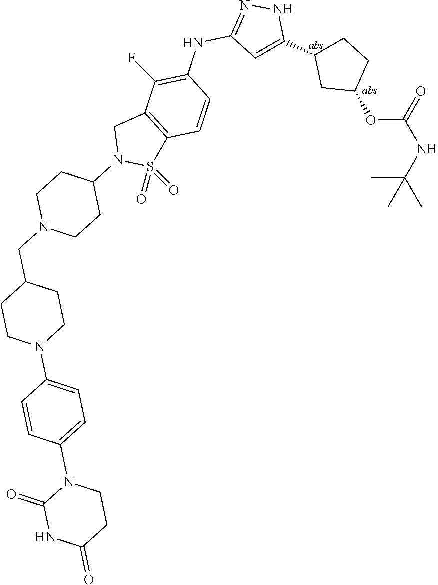 [SMILES: CC(C)(C)NC(=O)O[C@H]1CC[C@@H](c2cc(Nc3ccc4c(c3F)CN(C3CCN(CC5CCN(c6ccc(N7CCC(=O)NC7=O)cc6)CC5)CC3)S4(=O)=O)n[nH]2)C1]
or a pharmaceutically acceptable salt thereof.

30. The compound of claim 27, wherein the compound has the structure of 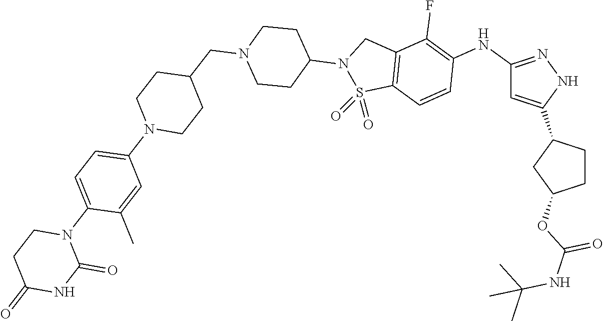 [SMILES: Cc1cc(N2CCC(CN3CCC(N4Cc5c(ccc(Nc6cc([C@@H]7CC[C@H](OC(=O)NC(C)(C)C)C7)[nH]n6)c5F)S4(=O)=O)CC3)CC2)ccc1N1CCC(=O)NC1=O]
or a pharmaceutically acceptable salt thereof.

31. The compound of claim 27, wherein the compound has the structure of 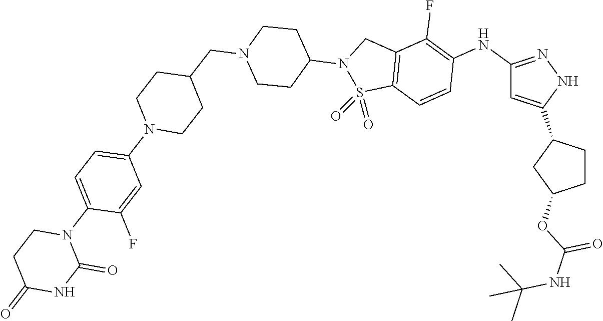 [SMILES: CC(C)(C)NC(=O)O[C@H]1CC[C@@H](c2cc(Nc3ccc4c(c3F)CN(C3CCN(CC5CCN(c6ccc(N7CCC(=O)NC7=O)c(F)c6)CC5)CC3)S4(=O)=O)n[nH]2)C1]
or a pharmaceutically acceptable salt thereof.

32. The compound of claim 27, wherein the compound has the structure of 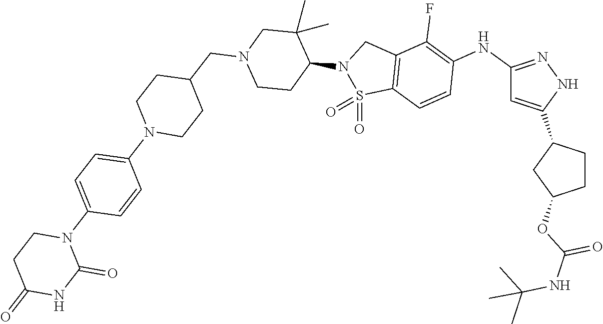 [SMILES: CC(C)(C)NC(=O)O[C@H]1CC[C@@H](c2cc(Nc3ccc4c(c3F)CN([C@H]3CCN(CC5CCN(c6ccc(N7CCC(=O)NC7=O)cc6)CC5)CC3(C)C)S4(=O)=O)n[nH]2)C1]
or a pharmaceutically acceptable salt thereof.

33. The compound of claim 27, wherein the compound has the structure of 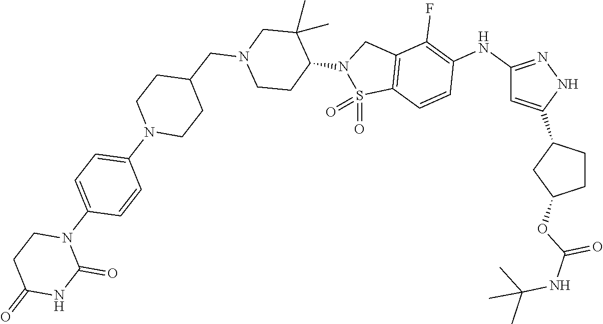 [SMILES: CC(C)(C)NC(=O)O[C@H]1CC[C@@H](c2cc(Nc3ccc4c(c3F)CN([C@@H]3CCN(CC5CCN(c6ccc(N7CCC(=O)NC7=O)cc6)CC5)CC3(C)C)S4(=O)=O)n[nH]2)C1]
or a pharmaceutically acceptable salt thereof.

34. The compound of claim 27, wherein the compound has the structure of 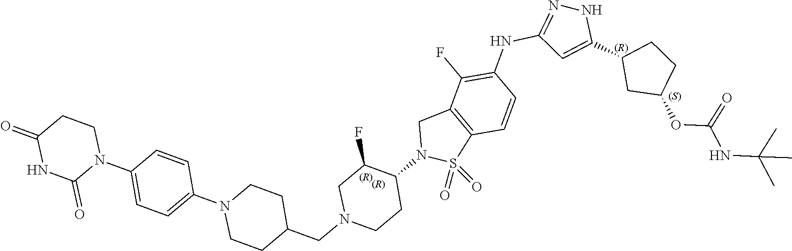
or a pharmaceutically acceptable salt thereof.

35. The compound of claim 27, wherein the compound has the structure of 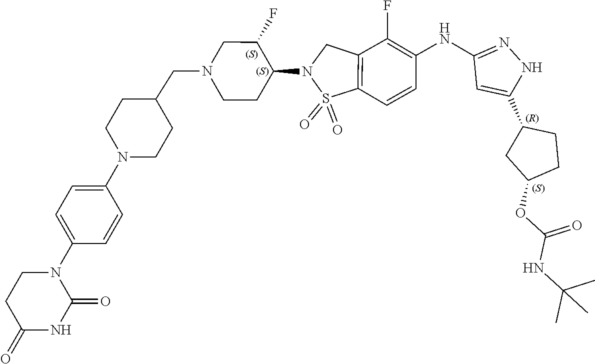
or a pharmaceutically acceptable salt thereof.

36. The compound of claim 27, wherein the compound has the structure of 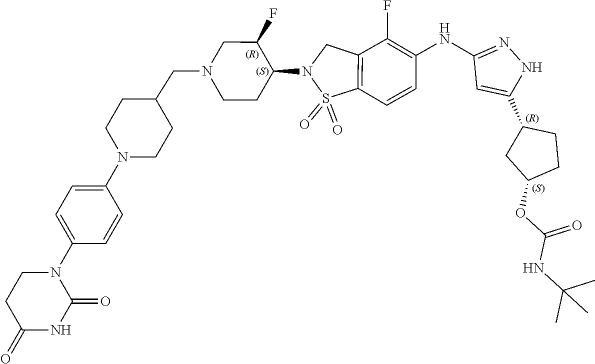
or a pharmaceutically acceptable salt thereof.

37. The compound of claim 27, wherein the compound has the structure of 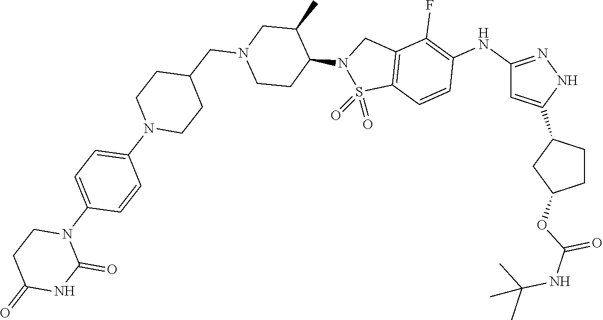 [SMILES: C[C@@H]1CN(CC2CCN(c3ccc(N4CCC(=O)NC4=O)cc3)CC2)CC[C@@H]1N1Cc2c(ccc(Nc3cc([C@@H]4CC[C@H](OC(=O)NC(C)(C)C)C4)[nH]n3)c2F)S1(=O)=O]
or a pharmaceutically acceptable salt thereof.

38. The compound of claim 27, wherein the compound has the structure of 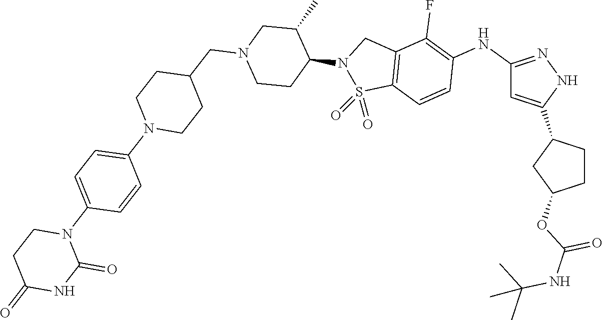 [SMILES: C[C@H]1CN(CC2CCN(c3ccc(N4CCC(=O)NC4=O)cc3)CC2)CC[C@@H]1N1Cc2c(ccc(Nc3cc([C@@H]4CC[C@H](OC(=O)NC(C)(C)C)C4)[nH]n3)c2F)S1(=O)=O]
or a pharmaceutically acceptable salt thereof.

39. The compound of claim 27, wherein the compound has the structure of 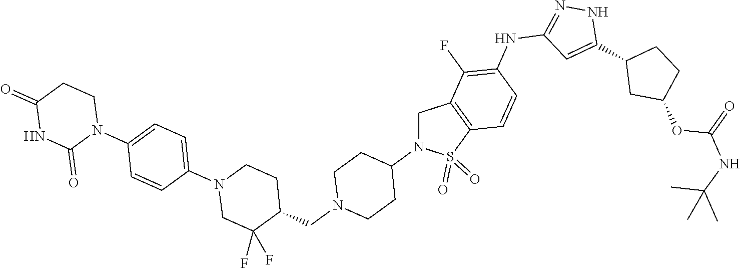 [SMILES: CC(C)(C)NC(=O)O[C@H]1CC[C@@H](c2cc(Nc3ccc4c(c3F)CN(C3CCN(C[C@H]5CCN(c6ccc(N7CCC(=O)NC7=O)cc6)CC5(F)F)CC3)S4(=O)=O)n[nH]2)C1]
or a pharmaceutically acceptable salt thereof.

40. The compound of claim 27, wherein the compound has the structure of 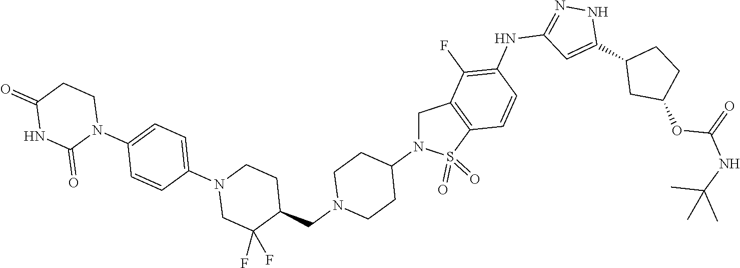 [SMILES: CC(C)(C)NC(=O)O[C@H]1CC[C@@H](c2cc(Nc3ccc4c(c3F)CN(C3CCN(C[C@@H]5CCN(c6ccc(N7CCC(=O)NC7=O)cc6)CC5(F)F)CC3)S4(=O)=O)n[nH]2)C1]
or a pharmaceutically acceptable salt thereof.

41. The compound of claim 27, wherein the compound has the structure of 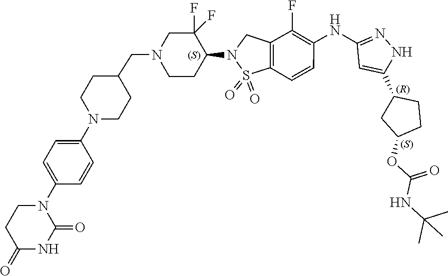
or a pharmaceutically acceptable salt thereof.

42. The compound of claim 27, wherein the compound has the structure of 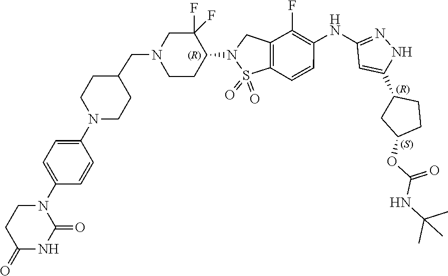
or a pharmaceutically acceptable salt thereof.

43. The compound of claim 27, wherein the compound has the structure of 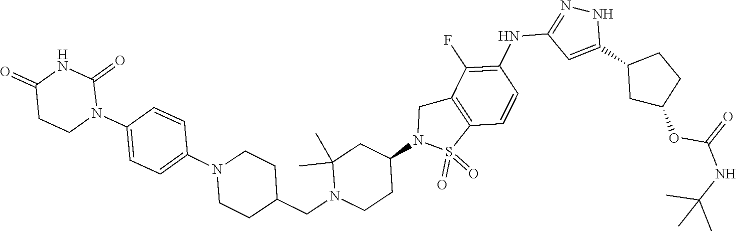 [SMILES: CC(C)(C)NC(=O)O[C@H]1CC[C@@H](c2cc(Nc3ccc4c(c3F)CN([C@H]3CCN(CC5CCN(c6ccc(N7CCC(=O)NC7=O)cc6)CC5)C(C)(C)C3)S4(=O)=O)n[nH]2)C1]
or a pharmaceutically acceptable salt thereof.

44. The compound of claim 27, wherein the compound has the structure of 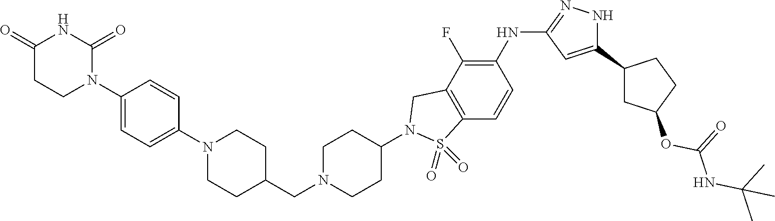 [SMILES: CC(C)(C)NC(=O)O[C@@H]1CC[C@H](c2cc(Nc3ccc4c(c3F)CN(C3CCN(CC5CCN(c6ccc(N7CCC(=O)NC7=O)cc6)CC5)CC3)S4(=O)=O)n[nH]2)C1]
or a pharmaceutically acceptable salt thereof.

45. The compound of claim 27, wherein the compound has the structure of 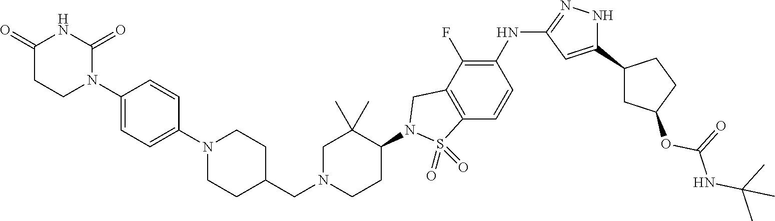 [SMILES: CC(C)(C)NC(=O)O[C@@H]1CC[C@H](c2cc(Nc3ccc4c(c3F)CN([C@H]3CCN(CC5CCN(c6ccc(N7CCC(=O)NC7=O)cc6)CC5)CC3(C)C)S4(=O)=O)n[nH]2)C1]
or a pharmaceutically acceptable salt thereof.

46. The compound of claim 27, wherein the compound has the structure of 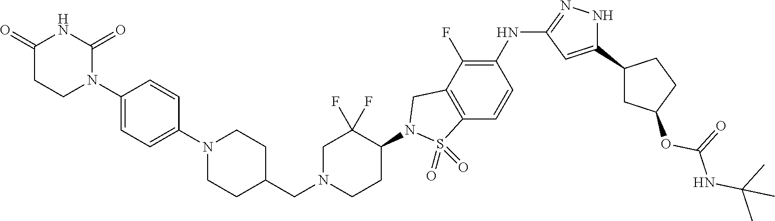 [SMILES: CC(C)(C)NC(=O)O[C@@H]1CC[C@H](c2cc(Nc3ccc4c(c3F)CN([C@H]3CCN(CC5CCN(c6ccc(N7CCC(=O)NC7=O)cc6)CC5)CC3(F)F)S4(=O)=O)n[nH]2)C1]
or a pharmaceutically acceptable salt thereof.

47. The compound of claim 27, wherein the compound has the structure of 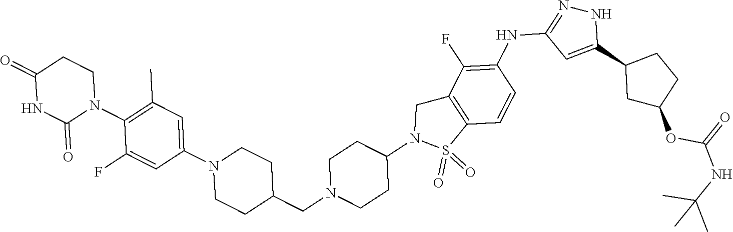 [SMILES: Cc1cc(N2CCC(CN3CCC(N4Cc5c(ccc(Nc6cc([C@H]7CC[C@@H](OC(=O)NC(C)(C)C)C7)[nH]n6)c5F)S4(=O)=O)CC3)CC2)cc(F)c1N1CCC(=O)NC1=O]
or a pharmaceutically acceptable salt thereof.

48. The compound of claim 27, wherein the compound has the structure of 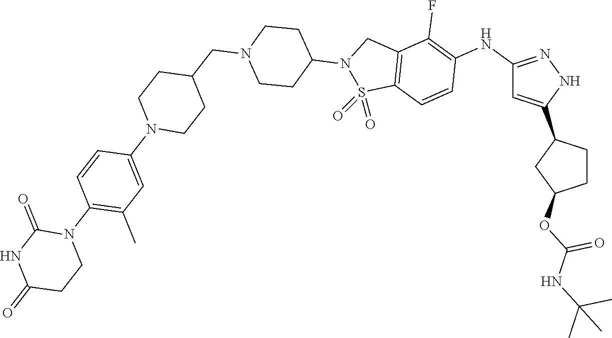 [SMILES: Cc1cc(N2CCC(CN3CCC(N4Cc5c(ccc(Nc6cc([C@H]7CC[C@@H](OC(=O)NC(C)(C)C)C7)[nH]n6)c5F)S4(=O)=O)CC3)CC2)ccc1N1CCC(=O)NC1=O]
or a pharmaceutically acceptable salt thereof.

49. The compound of claim 27, wherein the compound has the structure of 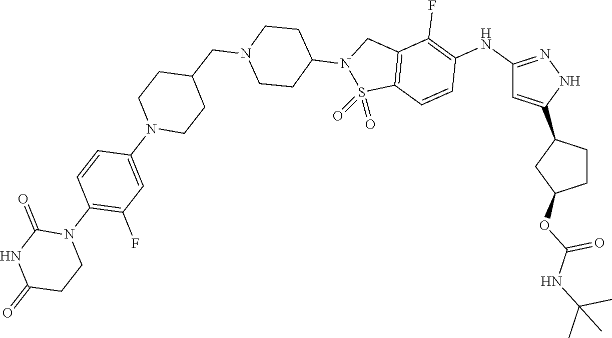 [SMILES: CC(C)(C)NC(=O)O[C@@H]1CC[C@H](c2cc(Nc3ccc4c(c3F)CN(C3CCN(CC5CCN(c6ccc(N7CCC(=O)NC7=O)c(F)c6)CC5)CC3)S4(=O)=O)n[nH]2)C1]
or a pharmaceutically acceptable salt thereof.

50. The compound of claim 27, wherein the compound has the structure of 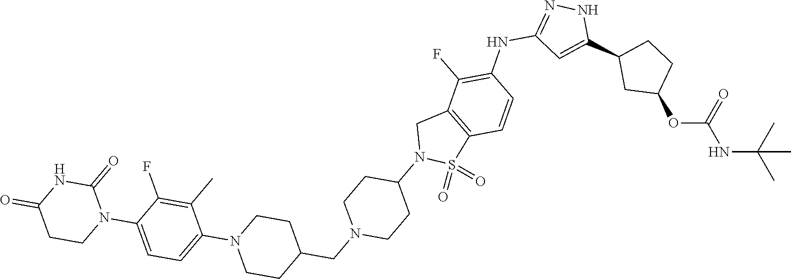 [SMILES: Cc1c(N2CCC(CN3CCC(N4Cc5c(ccc(Nc6cc([C@H]7CC[C@@H](OC(=O)NC(C)(C)C)C7)[nH]n6)c5F)S4(=O)=O)CC3)CC2)ccc(N2CCC(=O)NC2=O)c1F]
or a pharmaceutically acceptable salt thereof.

51. The compound of claim 27, wherein the compound has the structure of 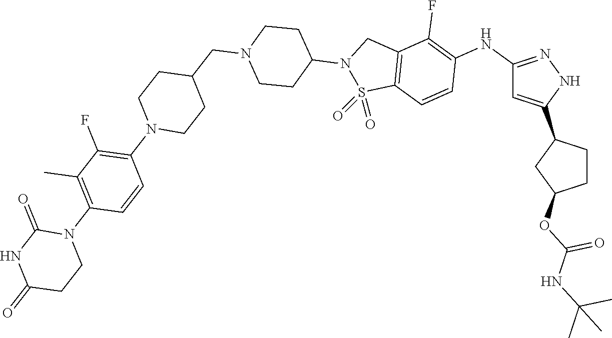 [SMILES: Cc1c(N2CCC(=O)NC2=O)ccc(N2CCC(CN3CCC(N4Cc5c(ccc(Nc6cc([C@H]7CC[C@@H](OC(=O)NC(C)(C)C)C7)[nH]n6)c5F)S4(=O)=O)CC3)CC2)c1F]
or a pharmaceutically acceptable salt thereof.
